(19) United States
(12) Patent Application Publication
(10) Pub. No.: US 2025/0289789 A1
(43) Pub. Date: Sep. 18, 2025

(54) REVERSIBLE LYSINE COVALENT MODIFIERS OF CDK2 AND USES THEREOF

(71) Applicant: Terremoto Biosciences, Inc., South San Francisco, CA (US)

(72) Inventors: Kin S. Yang, San Mateo, CA (US); Hang Chu, San Mateo, CA (US); Solomon H. Reisberg, Contra Costa, CA (US); Wai Yan Choy, Hayward, CA (US); Brian P. Bestvater, South San Francisco, CA (US); Adam Zajdlik, San Francisco, CA (US); Wayne Spevak, Alameda, CA (US)

(21) Appl. No.: 19/222,770

(22) Filed: May 29, 2025

Related U.S. Application Data
(63) Continuation of application No. PCT/US2024/052642, filed on Oct. 23, 2024.
(60) Provisional application No. 63/545,413, filed on Oct. 24, 2023.
(60) Provisional application No. 63/674,639, filed on Jul. 23, 2024.

(51) Int. Cl. C07D 231/40; A61K 31/4155; A61K 31/421; A61K 31/426; A61K 31/4439; A61K 31/4985; A61K 31/4995; A61K 31/5386; C07D 401/12; C07D 401/14; C07D 403/12; C07D 405/12; C07D 413/12; C07D 417/12; C07D 487/04; C07D 487/10; C07D 498/08

(52) U.S. Cl. CPC C07D 231/40; A61K 31/4155; A61K 31/421; A61K 31/426; A61K 31/4439; A61K 31/4985; A61K 31/4995; A61K 31/5386; C07D 401/12; C07D 401/14; C07D 403/12; C07D 405/12; C07D 413/12; C07D 417/12; C07D 487/04; C07D 487/10; C07D 498/08

(57) ABSTRACT

In some aspects, the present disclosure provides compounds of Formula (I), (II), (II-A), (II-B), (II-C), (II-D), (II-E), (II-F), (III), (IV), (IV-A), (IV-B), (IV-C), or (V), or pharmaceutically acceptable salts thereof, for the modulation of CDK2. In another aspect, the present disclosure provides methods for the treatment of diseases or disorders mediated by CDK2, such as oncology indications, using compounds of Formula (I), (II), (II-A), (II-B), (II-C), (II-D), (II-E), (II-F), (III), (IV), (IV-A), (IV-B), (IV-C), or (V), or pharmaceutically acceptable salts thereof.

### CROSS REFERENCE

This application is a continuation application of International Patent Application No. PCT/US2024/052642, filed Oct. 23, 2024, which claims the benefit of U.S. Provisional Application No. 63/545,413 filed on Oct. 24, 2023, and U.S. Provisional Application No. 63/674,639 filed on Jul. 23, 2024, the entirety of each is incorporated herein by reference.

### BACKGROUND OF THE INVENTION

Cyclin-Dependent Kinases (CDK) are a family of serine/threonine kinases involved in cell-cycle progression and cell division. The deregulation of CDK activity is associated with abnormal regulation of the cell cycle and is detected nearly all forms of human cancers (Sherr, C. J. Science, 1996, 274(5293), 1672-1677). Activation of CDK's requires the heterodimerzation with other regulatory subunits, these dimers known as cyclins. One of these, Cyclin E (a CDK2 regulator) is frequently overexpressed in cancer. Its amplification or overexpression has long been associated with poor outcomes in breast cancer, inflammatory breast cancer, and trastuzmab resistance in HER2+ breast cancers (Keyomarsi, K. et al., N Engl J Med., 2002, 347, 1566-1575; Scaltriti, M. et al., Proc Natl Acad Sci. 2011, 108, 3761-3766; Alexander, A. et al., Oncotarget 2017, 8, 14897-14911). Additionally, cyclin E1 amplification or overexpression is associated with poor outcomes in ovarian, gastric, endometrial and other cancers (Nakayama, N. et al., Cancer 2010, 116, 2621-2634; Etemadmoghadam, D. et al., Clin Cancer Res 2013, 19, 5960-5971). However, developing CDK2 selective inhibitors has been difficult due to its similarity to other CDK's. To date there have been no approved agents selectively targeting CDK2. Accordingly, there is need to develop new selective CDK2 modulators for oncology.

### SUMMARY OF THE INVENTION

In one aspect, the present disclosure provides a compound represented by the structure of Formula (I)

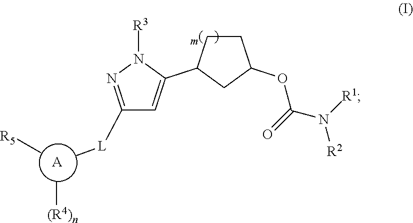

or a pharmaceutically acceptable salt thereof wherein:
Ring A is selected from C₃₋₁₂ carbocycle and 3- to 12-membered heterocycle;
R⁵ is selected from an electrophilic moiety or a prodrug thereof;
each R⁴ is independently selected at each occurrence from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
L is represented by -L¹-L²-L³-L⁴-, wherein L¹, L², L³, and L⁴ are each independently selected from (a) and (b):
   (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
   (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN;
   wherein L², L³, and L⁴ are each optionally absent;
   wherein no more than two of L¹, L², L³, and L⁴ are selected from (a) and the two selected are not adjacent;
R¹ and R² are each independently selected from (i), (ii), and (iii):
   (i) hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN;
   (ii) C₁₋₆ alkyl, C₂₋₆ alkenyl, and C₂₋₆ alkynyl, each of which is optionally substituted with one or more substituents independently selected from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
      C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
   (iii) C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; or
R¹ and R² may come together to form a 3- to 10-membered heterocycle optionally substituted with one or more substituents independently selected from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN; and
   C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN;
R³ is selected from:
   hydrogen, halogen, —OR¹⁶, —SR¹⁶, —N(R¹⁶)₂, —C(O)R¹⁶, —C(O)N(R¹⁶)₂, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —C(O)OR¹⁶, —NO₂, and —CN;
R¹¹, R¹², R¹³, R¹⁴, R, and R¹⁶ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 0, 1, 2, 3, and 4; and
m is selected from 0 and 1.

In some embodiments, the electrophilic moiety or a prodrug thereof is a lysine reactive electrophile. In some embodiments, the lysine reactive electrophile is selected from a reversible lysine reactive electrophile and an irreversible lysine reactive electrophile. In some embodiments, wherein the electrophilic moiety is selected from a haloacetamide, a haloalkyl ketone, a halo amidine, a halo benzylphosphonate, an acyloxyalkyl ketone, a sulfonyl oxirane, an epoxide, a diazoalkyl ketone, a halotriazine, an acrylamide, a cyano acrylamide, a vinyl sulfone, a vinyl sulfonamide, an acrylate, an aldehyde, a borate, a halosulfonyl, an ester, squaric ester, squaramide ester, a fumarate, a carbonyl acrylate, a maleimide, a ketoamide, a nitrile, an alkene, an alkyne, a keto heterocycle, and an ynamide. In some embodiments, the electrophilic moiety or prodrug thereof is an aldehyde or a prodrug thereof. In some embodiments, the electrophilic moiety or prodrug thereof is an imine. In some embodiments, the electrophilic moiety or prodrug thereof is an aldehyde.

In another aspect, a compound or salt of Formula (I) is represented by the structure of Formula (II):

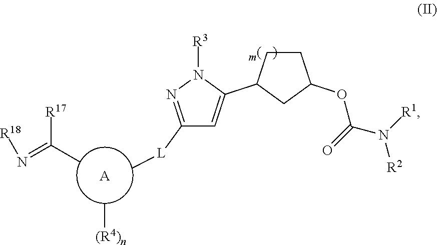

or a pharmaceutically acceptable salt thereof.

In another aspect, a compound or salt of Formula (I) is represented by the structure of Formula (III):

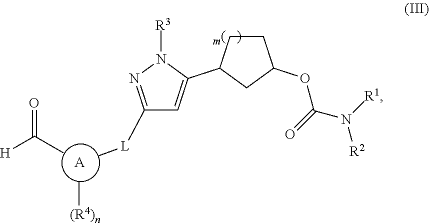

or a pharmaceutically acceptable salt thereof.

In one aspect, the present disclosure provides a pharmaceutical composition comprising a pharmaceutically acceptable excipient and a compound of Formula (I), (II), (II-A), (II-B), (II-C), (II-D), (II-E), (II-F), (III), (IV), (IV-A), (IV-B), (IV-C), or (V), or a pharmaceutically acceptable salt thereof.

In another aspect, the present disclosure provides a method of modulating CDK2 comprising administering to a subject in need thereof a compound or salt of Formula (I), (II), (II-A), (II-B), (II-C), (II-D)), (II-E), (II-F), (III), (IV), (IV-A), (IV-B), (IV-C), or (V), or a pharmaceutical composition thereof.

In another aspect, the present disclosure provides a method of inhibiting CDK2 comprising administering to a subject in need thereof a compound or salt of Formula (I), (II), (II-A), (II-B), (II-C), (II-D)), (II-E), (II-F), (III), (IV), (IV-A), (IV-B), (IV-C), or (V), or a pharmaceutical composition thereof.

In another aspect, the present disclosure provides a method of treating a disease or disorder mediated by CDK2 comprising administering to a subject in need thereof a compound or salt of Formula (I), (II), (II-A), (II-B), (II-C), (II-D), (II-E), (II-F), (III), (IV), (IV-A), (IV-B), (IV-C), or (V), or a pharmaceutical composition thereof.

In another aspect, the present disclosure provides a method of treating cancer comprising administering to a subject in need thereof a compound or salt of Formula (I), (II), (II-A), (II-B), (II-C), (II-D), (II-E), (II-F), (III), (IV), (IV-A), (IV-B), (IV-C), or (V), or a pharmaceutical composition thereof.

In another aspect, the present disclosure provides a method of inhibiting cell proliferation comprising administering to a subject in need thereof a compound or salt of Formula (I), (II), (II-A), (II-B), (II-C), (II-D)), (II-E), (II-F), (III), (IV), (IV-A), (IV-B), (IV-C), or (V), or a pharmaceutical composition thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the instant claims. A better understanding of the features and advantages of the present invention will be obtained by reference to the following detailed description that sets forth illustrative embodiments, in which the principles of the invention are utilized, and the accompanying drawings of which:

FIGS. 1A-1P provide intact-protein mass spectra for pCDK2-CCNE1 incubated with compound of the present disclosure both without a competitor molecule and with a significant stoichiometric excess of a competitor molecule (PF-06873600). FIGS. 1A-1B provide intact-protein mass spectra for pCDK2-CCNE1 incubated with Compound 11, both without a competitor and with a significant stoichiometric excess of a competitor molecule PF-06873600, respectively. FIGS. 1C-1D provide intact-protein mass spectra for pCDK2-CCNE1 incubated with Compound 13, both without a competitor and with a significant stoichiometric excess of a competitor molecule PF-06873600, respectively. FIGS. 1E-1F provide intact-protein mass spectra for pCDK2-CCNE1 incubated with Compound 17, both without a competitor and with a significant stoichiometric excess of a competitor molecule PF-06873600, respectively. FIGS. 1G-1H provide intact-protein mass spectra for pCDK2-CCNE1 incubated with Compound 17, both without a competitor and with a significant stoichiometric excess of a competitor molecule PF-06873600, respectively. FIGS. 1I-1J provide intact-protein mass spectra for pCDK2-CCNE1 incubated with Compound 22, both without a competitor and with a significant stoichiometric excess of a competitor molecule PF-06873600, respectively. FIGS. 1K-1L provide intact-protein mass spectra for pCDK2-CCNE1 incubated with Compound 51, both without a competitor and with a significant stoichiometric excess of a competitor molecule PF-06873600, respectively. FIGS. 1M-1N provide intact-protein mass spectra for pCDK2-CCNE1 incubated with Compound 69, both without a competitor and with a significant stoichiometric excess of a competitor molecule PF-06873600, respectively. FIGS. 10-1P provide intact-protein mass spectra for pCDK2-CCNE1 incubated with Compound 32, both without a competitor and with a significant stoichiometric excess of a competitor molecule PF-06873600, respectively.

FIGS. 2A-2N provide intact-protein mass spectra for pCDK2-CCNE1 proteins after incubation with compounds of the present disclosure and subsequent sodium borohydride reduction. FIG. 2A provides intact-protein mass spectra for pCDK2-CCNE1 incubated with Compound 11. FIG. 2B provides intact-protein mass spectra for pCDK2-CCNE1 incubated with Compound 13. FIG. 2C provides intact-protein mass spectra for pCDK2-CCNE1 incubated with Compound 17. FIG. 2D provides intact-protein mass spectra for pCDK2-CCNE1 incubated with Compound 22. FIG. 2E provides intact-protein mass spectra for pCDK2-CCNE1 incubated with Compound 16. FIG. 2F provides intact-protein mass spectra for pCDK2-CCNE1 incubated with Compound 51. FIG. 2G provides intact-protein mass spectra for pCDK2-CCNE1 incubated with Compound 64. FIG. 2H provides intact-protein mass spectra for pCDK2-CCNE1 incubated with Compound 69. FIG. 2I provides intact-protein mass spectra for pCDK2-CCNE1 incubated with Compound 45. FIG. 2J provides intact-protein mass spectra for pCDK2-CCNE1 incubated with Compound 32. FIG. 2K provides intact-protein mass spectra for pCDK2-CCNE1 incubated with Compound 49. FIG. 2L provides intact-protein mass spectra for pCDK2-CCNE1 incubated with Compound 82. FIG. 2M provides intact-protein mass spectra for pCDK2-CCNE1 incubated with Compound 89. FIG. 2N provides intact-protein mass spectra for pCDK2-CCNE1 incubated with Compound 103.

FIGS. 3A-3N provide dissociation rate data for pCDK2-CCNE1 treated with compounds of the present disclosure via competition with excess PF-06873600. FIG. 3A illustrates off-rate studies of Compound 11 with pCDK2-CCNE1. FIG. 3B illustrates off-rate studies of Compound 13 with pCDK2-CCNE1. FIG. 3C illustrates off-rate studies of Compound 17 with pCDK2-CCNE1. FIG. 3D illustrates off-rate studies of Compound 22 with pCDK2-CCNE1. FIG. 3E illustrates off-rate studies of Compound 16 with pCDK2-CCNE1. FIG. 3F illustrates off-rate studies of Compound 51 with pCDK2-CCNE1. FIG. 3G illustrates off-rate studies of Compound 64 with pCDK2-CCNE1. FIG. 3H illustrates off-rate studies of Compound 69 with pCDK2-CCNE1. FIG. 3I illustrates off-rate studies of Compound 45 with pCDK2-CCNE1. FIG. 3J illustrates off-rate studies of Compound 32 with pCDK2-CCNE1. FIG. 3K illustrates off-rate studies of Compound 49 with pCDK2-CCNE1. FIG. 3L illustrates off-rate studies of Compound 82 with pCDK2-CCNE1. FIG. 3M illustrates off-rate studies of Compound 89 with pCDK2-CCNE1. FIG. 3N illustrates off-rate studies of Compound 103 with pCDK2-CCNE1.

FIGS. 4A-4C provide diagrams of the co-crystal structure for the pCDK2-CCNE1 complex with Compound 13. FIG. 4A provides a 2-D diagram from the co-crystal structure of the pCDK2-CCNE1 complex with Compound 13 with the pCDK2-CCNE1 rendered in cartoon model and Compound 13 with the CDK2 lysine residue K89 rendered in stick model. FIG. 4B provides a 2-D diagram close-up from the co-crystal structure of the pCDK2-CCNE1 complex with Compound 13 depicted by a thicker stick model and the residues pCDK2-CCNE1 depicted by a thinner stick model. FIG. 4C provides a 2-D diagram close-up from the co-crystal structure of the pCDK2-CCNE1 complex with Compound 13 detailing the interactions between the residues of CDK2 and Compound 13, specifically showing the imine bond between K89 lysine residue of CDK2 and Compound 13.

FIGS. 5A-5C provide diagrams of the co-crystal structure for the pCDK2-CCNE1 complex with Compound 17. FIG. 5A provides a 2-D diagram from the co-crystal structure of the pCDK2-CCNE1 complex with Compound 17 with the pCDK2-CCNE1 rendered in cartoon model and Compound 17 with the CDK2 lysine residue K89 rendered in stick model. FIG. 5B provides a 2-D diagram close-up from the co-crystal structure of the pCDK2-CCNE1 complex with Compound 17 with Compound 17 depicted by a thicker stick model and the residues pCDK2-CCNE1 depicted by a thinner stick model. FIG. 5C provides a 2-D diagram close-up from the co-crystal structure of the pCDK2-CCNE1 complex with Compound 17 detailing the interactions between the residues of CDK2 and Compound 17, specifically showing the imine bond between K89 lysine residue of CDK2 and Compound 17.

FIGS. 6A-6C provide diagrams of the co-crystal structure for the pCDK2-CCNE1 complex with Compound 64. FIG. 6A provides a 2-D diagram from the co-crystal structure of the pCDK2-CCNE1 complex with Compound 64 the pCDK2-CCNE1 rendered in cartoon model and Compound 64 with the CDK2 lysine residue K89 rendered in stick model. FIG. 6B provides a 2-D diagram close-up from the co-crystal structure of the pCDK2-CCNE1 complex with Compound 64 depicted by a thicker stick model and the residues pCDK2-CCNE1 depicted by a thinner stick model. FIG. 6C provides a 2-D diagram close-up from the cocrystal structure of the pCDK2-CCNE1 complex with Compound 64 detailing the interactions between the residues of CDK2 and Compound 64, specifically showing the imine bond between K89 lysine residue of CDK2 and Compound 64.

FIGS. 7A-7B provide plasma concentrations for compounds 64 and 64a following oral and IV administration to mice. FIG. 7A shows plasma concentrations for compounds 64 and 64a following oral administration to mice. FIG. 7B shows plasma concentrations for compounds 64 and 64 following IV administration to mice.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene-refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene- refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene-refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the bicyclic carbocycle is selected from fused carbocycle, bridged carbocycle, and spirocyclic carbocycle. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

The term “carbocyclene” as used herein refers to a divalent saturated, unsaturated or aromatic ring in which each atom of the ring is carbon. The carbocyclene is attached to the rest of the molecule through a single bond and to the radical group through a single bond. A carbocyclene may be optionally substituted by one or more substituents such as those substituents described herein. Carbocyclene includes divalent 3- to 10-membered monocyclic rings and divalent polycyclic rings (e.g., 6- to 12-membered bicyclic rings). Each ring of a polycyclic carbocyclene may be selected from saturated, unsaturated, and aromatic rings. Polycyclic carbocyclenes may be fused, bridged or spiro-ring systems. Polycyclic carbocyclenes may be fused, bridged or spiro-ring systems. The single bond connecting the carbocyclene to the rest of the molecule and the single bond connecting the carbocyclene to the radical group may be located on two different carbon atoms of the carbocyclene. The single bond connecting the carbocyclene to the rest of the molecule and the single bond connecting the carbocyclene to the radical group may be located on the same carbon atom of the carbocyclene. The single bond connecting the carbocyclene to the rest of the molecule and the single bond connecting the carbocyclene to the radical group may be located on the same ring or different rings of a polycyclic carbocyclene. In some embodiments, the carbocycle is an arylene, for example, a phenylene. A “phenylene” as used herein refers to a divalent benzene group. The phenylene is attached to the rest of the molecule through a single bond and to the radical group through a single bond. A phenylene may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

The term “heterocyclene” as used herein refers to a divalent saturated, unsaturated, non-aromatic or aromatic ring comprising one or more heteroatoms. Exemplary heteroatoms include N, O, Si, P, B, and S atoms. The heterocyclene is attached to the rest of the molecule through a single bond and to the radical group through a single bond. The single bond attaching the heterocyclene group to the rest of the molecule and the single bond attaching the heterocyclene group to the radical group may be each independently connected through any atom of the heterocyclene as valency permits, including a carbon atom in the heterocyclene ring or a heteroatom in the heterocyclene ring. A heterocyclene may be optionally substituted by one or more substituents such as those substituents described herein. Heterocyclenes include 3- to 10-membered monocyclic rings and polycyclic rings (e.g., 6- to 12-membered bicyclic rings). Each ring of a polycyclic heterocyclene may be selected from saturated, unsaturated, and aromatic rings. Polycyclic heterocyclenes may be fused, bridged or spiro-ring systems. The single bond connecting the heterocyclene to the rest of the molecule and the single bond connecting the heterocyclene to the radical group may be located on two different carbon atoms of the heterocyclene. The single bond connecting the heterocyclene to the rest of the molecule and the single bond connecting the heterocyclene to the radical group may be located on the same carbon atom of the heterocyclene. The single bond connecting the heterocyclene to the rest of the molecule and the single bond connecting the heterocyclene to the radical group may be located on the same ring or different rings of a polycyclic heterocyclene and may be attached to the rest of the molecule or the radical group through any atom of the heterocyclene, valence permitting, such as a carbon or nitrogen atom of the heterocycle. In some embodiments, the heterocyclene comprises at least one heteroatom selected from oxygen, nitrogen, sulfur, or any combination thereof. In some embodiments, the heterocyclene comprises at least one heteroatom selected from oxygen, nitrogen, or any combination thereof. In some embodiments, the heterocyclene comprises at least one heteroatom selected from oxygen, sulfur, or any combination thereof. In some embodiments, the heterocyclene comprises at least one heteroatom selected from nitrogen, sulfur, or any combination thereof. In some embodiments, the heterocyclene is a heteroarylene. In some embodiments, the heterocyclene is a heterocycloalkylene.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 5- to 12-membered aromatic ring radical whose ring structure comprise at least one heteroatom, preferably between one to four heteroatoms. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. One or more nitrogen atoms, if present, are optionally quaternized. The heteroaryl may be attached to the rest of the molecule through any atom of the heteroaryl, valence permitting, such as a carbon or nitrogen atom of the heteroaryl.

As used herein, the heteroaryl ring may be selected from monocyclic or polycyclic (bicyclic and fused or bridged) systems rings wherein at least one of the rings in the ring system is aromatic, i.e., it contains a cyclic, delocalized (4n+2) π-electron system in accordance with the Hückel theory. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro,2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the invention or a prodrug of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition and can contemplate a range of results directed to that end, including but not restricted to prevention of the condition entirely.

As used herein, “PF-06873600” is a competitor molecule. In some embodiments, the competitor molecule is molecule represented by the structure of

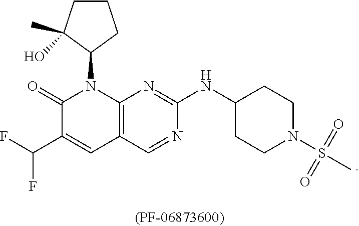 [SMILES: C[C@@]1(O)CCC[C@H]1n1c(=O)c(C(F)F)cc2cnc(NC3CCN(S(C)(=O)=O)CC3)nc21]

In some embodiments, PF-06873600 is

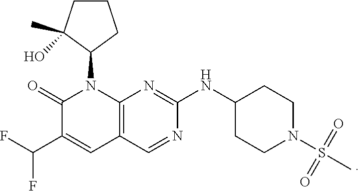 [SMILES: C[C@@]1(O)CCC[C@H]1n1c(=O)c(C(F)F)cc2cnc(NC3CCN(S(C)(=O)=O)CC3)nc21]

In some embodiments, the competitor molecule is 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one (PF-06873600).

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

### Compounds

In some aspects, the present disclosure provides a compound represented by the structure of Formula (I)

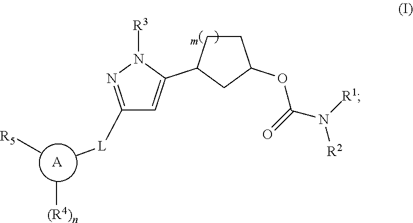

or a pharmaceutically acceptable salt thereof wherein:
Ring A is selected from C₃₋₁₂ carbocycle and 3- to 12-membered heterocycle;
R⁵ is selected from an electrophilic moiety or a prodrug thereof;
each R⁴ is independently selected at each occurrence from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
L is represented by -L¹-L²-L³-L⁴-, wherein L¹, L², L³, and L⁴ are each independently selected from (a) and (b):
   (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
   (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN;
   wherein L², L³, and L⁴ are each optionally absent;
   wherein no more than two of L¹, L², L³, and L⁴ are selected from (a) and the two selected are not adjacent;
R¹ and R² are each independently selected from (i), (ii), and (iii):
   (i) hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN;
   (ii) C₁₋₆ alkyl, C₂₋₆ alkenyl, and C₂₋₆ alkynyl, each of which is optionally substituted with one or more substituents independently selected from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
      C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
   (iii) C₃₋₁₀ carbocycle and 3- to 10-membered heterocycle, each of which is optionally substituted with one or more substituents independently selected from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; or
R¹ and R² may come together to form a 3- to 10-membered heterocycle optionally substituted with one or more substituents independently selected from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN; and
   C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN;
R³ is selected from:
   hydrogen, halogen, —OR¹⁶, —SR¹⁶, —N(R¹⁶)₂, —C(O)R¹⁶, —C(O)N(R¹⁶)₂, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —C(O)OR¹⁶, —NO₂, and —CN;
R¹¹, R¹², R¹³, R¹⁴, R, and R¹⁶ are each independently selected at each occurrence from: hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 0, 1, 2, 3, and 4; and
m is selected from 0 and 1.

In some embodiments, for the compound or salt of Formula (I), the electrophilic moiety is a lysine reactive electrophile. In some embodiments, the lysine reactive electrophile is selected from a reversible lysine reactive electrophile and an irreversible lysine reactive electrophile.

In some embodiments, for the compound or salt of Formula (I), the electrophilic moiety is selected from a haloacetamide, a haloalkyl ketone, a halo amidine, a halo benzylphosphonate, an acyloxyalkyl ketone, a sulfonyl oxirane, an epoxide, a diazoalkyl ketone, a halotriazine, an acrylamide, a cyano acrylamide, a vinyl sulfone, a vinyl sulfonamide, an acrylate, an aldehyde, a borate, a halosulfonyl, an ester, squaric ester, squaramide ester, a fumarate, a carbonyl acrylate, a maleimide, a ketoamide, a nitrile, an alkene, an alkyne, a keto heterocycle, and an ynamide. In some embodiments, the electrophilic moiety is selected from a haloacetamide, a haloalkyl ketone, a halo amidine, a halo benzylphosphonate, an acyloxyalkyl ketone, a sulfonyl oxirane, an epoxide, a diazoalkyl ketone, a halotriazine, an acrylamide, a cyano acrylamide, a vinyl sulfone, a vinyl sulfonamide, an acrylate, a fumarate, a carbonyl acrylate, a maleimide, a ketoamide, a nitrile, an alkene, an alkyne, a keto heterocycle, and an ynamide. In some embodiments, the electrophilic moiety is selected an aldehyde, a borate, and a halosulfonyl.

In some embodiments, for the compound or salt of Formula (I), the electrophilic moiety or prodrug thereof is an aldehyde or a prodrug thereof. In some embodiments, the electrophilic moiety or prodrug thereof of R⁵ is an aldehyde or a prodrug thereof. In some embodiments, the electrophilic moiety or prodrug thereof of R⁵ is an aldehyde.

In some embodiments, for the compound or salt of Formula (I), the aldehyde or a prodrug thereof is an imine. In some embodiments, the electrophilic moiety or prodrug thereof of R⁵ is an imine.

In some embodiments, for the compound or salt of Formula (I), the imine is represented by —C(R¹⁷)═N(R¹⁸); wherein
R¹⁷ is selected from hydrogen and C₁₋₆ alkyl optionally substituted with one or more substituents independently selected from halogen, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)OR²⁰, —N(R²⁰)C(O)R²⁰, —S(O)R²⁰, —S(O)₂R²⁰, —N(R²⁰)S(O)₂R²⁰, —S(O)₂N(R²⁰)₂, —NO₂, ═O, ═S, ═N(R²⁰) and —CN;
R¹⁸ is selected from:
   hydrogen;
   C₁₋₆ alkyl optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —P(═O)(OR²¹)₂, —NO₂, ═O, ═S, ═N(R²¹), —CN, C₃₋₁₀ carbocycle, and 3- to 10-membered heterocycle; and
   C₃₋₁₀ carbocycle and 3- to 10-membered heterocycle, any of which are optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —NO₂, ═O, ═S, ═N(R²¹), —CN, C₁₋₆ alkyl, and C₁₋₆ haloalkyl; and
R²⁰ and R²¹ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.

In some embodiments, for the compound or salt of Formula (I), R⁵ is an imine.

In some embodiments, for the compound or salt of Formula (I), R⁵ is —C(R¹⁷)═N(R¹⁸);
R¹⁷ is selected from hydrogen and C₁₋₆ alkyl optionally substituted with one or more substituents independently selected from halogen, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)OR²⁰, —N(R²⁰)C(O)R²⁰, —S(O)R²⁰, —S(O)₂R²⁰, —N(R²⁰)S(O)₂R²⁰, —S(O)₂N(R²⁰)₂, —NO₂, ═O, ═S, ═N(R²⁰) and —CN;
R¹⁸ is selected from:
   hydrogen;
   C₁₋₆ alkyl optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —P(═O)(OR²¹)₂, —NO₂, ═O, ═S, ═N(R²¹), —CN, C₃₋₁₀ carbocycle, and 3- to 10-membered heterocycle; and
   C₃₋₁₀ carbocycle and 3- to 10-membered heterocycle, any of which are optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —NO₂, ═O, ═S, ═N(R²¹), —CN, C₁₋₆ alkyl, and C₁₋₆ haloalkyl; and
R²⁰ and R²¹ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.

In certain embodiments, the compound or salt of Formula (I) or (II), is represented by the structure of Formula (II-A):

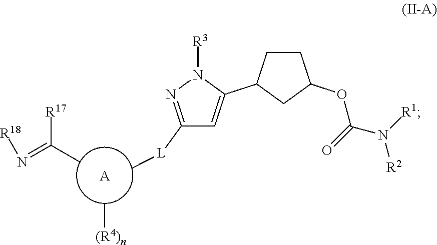

wherein Ring A, R¹, R², R³, R⁴, L, n, R¹⁷, and R¹⁸ are each defined as in Formula (I) or (II).

In certain embodiments, the compound or salt of Formula (I) or (II), is represented by the structure of Formula (II-B):

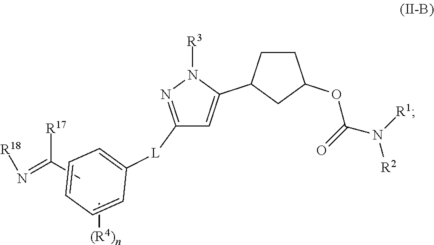

wherein R¹, R², R³, R⁴, L, n, R¹⁷, and R¹⁸ are each defined as in Formula (I) or (II).

In certain embodiments, the compound or salt of Formula (I) or (II), is represented by the structure of Formula (II-C):

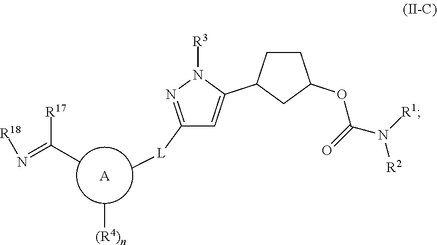

wherein Ring A is indanyl; and each of R¹, R², R³, R⁴, L, n, R¹⁷, and R¹⁸ are each defined as in Formula (I) or (II).

In certain embodiments, the compound or salt of Formula (I) or (II), is represented by the structure of Formula (II-D):

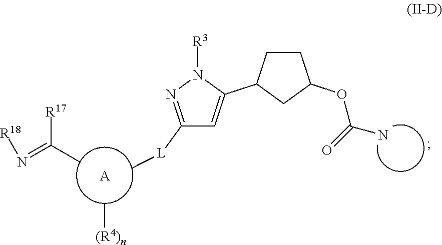

wherein R¹ and R² come together to form an optionally substituted a 3- to 10-membered heterocycle represented by

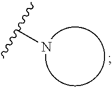 [SMILES: *N]

and Ring A, R³, R⁴, L, n, R¹⁷, and R¹⁸ are each defined as in Formula (I) or (II).

In certain embodiments, the compound or salt of Formula (I) or (II), is represented by the structure of Formula (II-E):

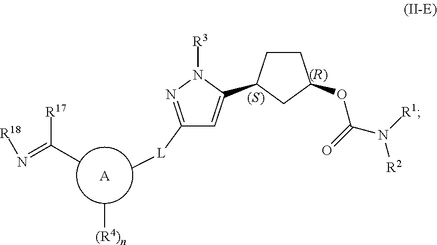

wherein Ring A, R¹, R², R³, R⁴, L, n, R¹⁷, and R¹⁸ are each defined as in Formula (I) or (II).

In certain embodiments, the compound or salt of Formula (I) or (II), is represented by the structure of Formula (II-F):

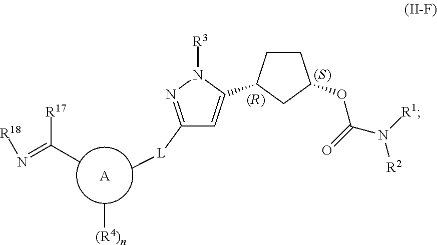

wherein Ring A, R¹, R², R³, R⁴, L, n, R¹⁷, and R¹⁸ are each defined as in Formula (I) or (II).

In some embodiments, for the compound or salt of Formula (II), (II-A), (II-B), (II-C), (II-D), (II-E), or (II-F), R¹⁷ is selected from hydrogen and C₁₋₆ alkyl optionally substituted with one or more substituents independently selected from halogen, —OR²⁰, —SR²⁰, —N(R²⁰)₂, —C(O)R²⁰, —C(O)N(R²⁰)₂, —C(O)OR²⁰, —OC(O)R²⁰, —N(R²⁰)C(O)OR²⁰, —N(R²⁰)C(O)R²⁰, —NO₂, ═O, and —CN. In some embodiments, R¹⁷ is selected from hydrogen and C₁₋₆ alkyl optionally substituted with one or more substituents independently selected from halogen, —OR²⁰, —SR²⁰, —N(R²⁰)₂, —C(O)R²⁰, —C(O)N(R²⁰)₂, —NO₂, ═O, and —CN. In some embodiments, R¹⁷ is selected from hydrogen and C₁₋₆ alkyl optionally substituted with one or more substituents independently selected from halogen, —OR²⁰, —SR²⁰, —N(R²⁰)₂, —NO₂, and —CN.

In some embodiments, for the compound or salt of Formula (II), (II-A), (II-B), (II-C), (II-D), (II-E), or (II-F), R¹⁷ is selected from hydrogen, C₁₋₆ alkyl, and C₁₋₆ haloalkyl. In some embodiments, R¹⁷ is selected from hydrogen, C₁₋₃ alkyl, and C₁₋₃ haloalkyl. In some embodiments, R¹⁷ is hydrogen. In some embodiments, R¹⁷ is C₁₋₆ alkyl. In some embodiments, R¹⁷ is C₁₋₃ alkyl. In some embodiments, R¹⁷ is C₁₋₆ haloalkyl. In some embodiments, R¹⁷ is C₁₋₃ haloalkyl.

In some embodiments, for the compound or salt of Formula (II), (II-A), (II-B), (II-C), (II-D), (II-E), or (II-F), R¹⁸ is hydrogen.

In some embodiments, for the compound or salt of Formula (II), (II-A), (II-B), (II-C), (II-D), (II-E), or (II-F), R¹⁸ is C₁₋₆ alkyl optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —P(═O)(OR²¹)₂, —NO₂, ═O, ═S, ═N(R²¹), —CN, C₃₋₁₀ carbocycle, and 3- to 10-membered heterocycle. In some embodiments, R¹⁸ is C₁₋₆ alkyl optionally substituted with one or more substituents independently selected from halogen, —OR²¹, —N(R²¹)₂, —C(O)R²¹—C(O)OR²¹, —OC(O)R²¹, —S(O)₂R²¹, —P(═O)(OR²¹)₂, —NO₂, —CN, C₃₋₁₀ carbocycle, and 3- to 10-membered heterocycle. In some embodiments, R¹⁸ is C₁₋₆ alkyl optionally substituted with one or more substituents independently selected from halogen, —OR²¹, —P(═O)(OR²¹)₂, —S(O)₂R²¹, —NO₂, —CN, C₃₋₆carbocycle, and 3- to 6-membered heterocycle.

In some embodiments, for the compound or salt of Formula (II), (II-A), (II-B), (II-C), (II-D), (II-E), or (II-F), R¹⁸ is selected from hydrogen; and C₁₋₆ alkyl optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —NO₂, ═O, —CN, C₃₋₁₀ carbocycle, and 3- to 10-membered heterocycle. In some embodiments, R¹⁸ is selected from hydrogen; and C₁₋₆ alkyl optionally substituted with one or more substituents independently selected from halogen, —OR²¹, —SR²¹, —N(R²¹)₂, —C(O)R²¹, —C(O)N(R²¹)₂, —NO₂, ═O, —CN, C₃₋₁₀ carbocycle, and 3- to 10-membered heterocycle. In some embodiments, R¹⁸ is selected from hydrogen; and C₁₋₆ alkyl optionally substituted with one or more substituents independently selected from halogen, —OR²¹, —SR²¹, —N(R²¹)₂, —NO₂, —CN, C₃₋₁₀ carbocycle, and 3- to 10-membered heterocycle. In some embodiments, R¹⁸ is selected from hydrogen; and C₁₋₆ alkyl optionally substituted with one or more substituents independently selected from halogen, —OR²¹, —SR²¹, —N(R²¹)₂, —NO₂, and —CN.

In some embodiments, for the compound or salt of Formula (II), (II-A), (II-B), (II-C), (II-D), (II-E), or (II-F), R¹⁸ is selected from hydrogen, C₁₋₆ alkyl, and C₁₋₆ haloalkyl.

In some embodiments, for the compound or salt of Formula (II), (II-A), (II-B), (II-C), (II-D), (II-E), or (II-F), R¹⁸ is selected from hydrogen,

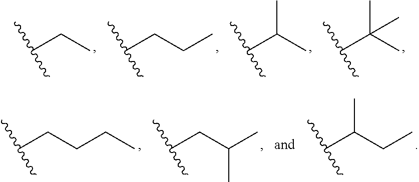

In some embodiments, for the compound or salt of Formula (II), (II-A), (II-B), (II-C), (II-D), (II-E), or (II-F), R¹⁸ is C₁₋₆ alkyl substituted with one or more substituents independently selected from —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —P(═O)(OR²¹)₂, —NO₂, ═O, ═S, ═N(R²¹), and —CN.

In some embodiments, for the compound or salt of Formula (II), (II-A), (II-B), (II-C), (II-D), (II-E), or (II-F), R¹⁸ is C₁₋₆ alkyl substituted with one or more substituents independently selected from —OR²¹, —N(R²¹)₂, —C(O)R²¹, —C(O)N(R²¹)₂, —C(O)OR²¹, —S(O)₂R²¹, —P(═O)(OR²¹)₂, —NO₂, ═O, and —CN.

In some embodiments, for the compound or salt of Formula (II), (II-A), (II-B), (II-C), (II-D), (II-E), or (II-F), R¹⁸ is C₁₋₆ alkyl substituted with one or more substituents independently selected from —OR²¹, —N(R²¹)₂, —C(O)R²¹, —C(O)N(R²¹)₂, —C(O)OR²¹, —S(O)₂R²¹, —P(═O)(OR²¹)₂, —NO₂, ═O, and —CN, wherein R²¹ is independently selected at each occurrence from hydrogen, C₁₋₆ alkyl, and C₁₋₆ haloalkyl.

In some embodiments, for the compound or salt of Formula (II), (II-A), (II-B), (II-C), (II-D), (II-E), or (II-F), R¹⁸ is selected from OOH

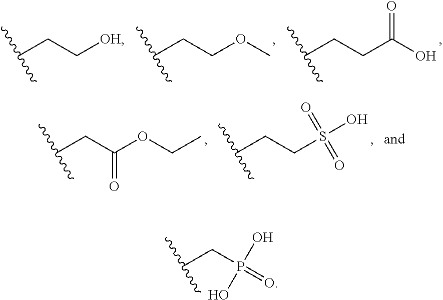

In some embodiments, for the compound or salt of Formula (II), (II-A), (II-B), (II-C), (II-D), (II-E), or (II-F), R¹⁸ is C₁₋₆ alkyl substituted with one or more substituents independently selected from C₃₋₁₀ carbocycle and 3- to 10-membered heterocycle. In some embodiments, R¹⁸ is C₁₋₆ alkyl substituted with one or more substituents independently selected from C₃₋₆ carbocycle and 3- to 6-membered heterocycle.

In some embodiments, for the compound or salt of Formula (II), (II-A), (II-B), (II-C), (II-D), (II-E), or (II-F), R¹⁸ is C₁₋₆ alkyl substituted with one or more C₃₋₆ carbocycle. In some embodiments, R¹⁸ is C₁₋₆ alkyl substituted with one or more phenyl.

In some embodiments, for the compound or salt of Formula (II), (II-A), (II-B), (II-C), (II-D), (II-E), or (II-F), R¹⁸ is selected from

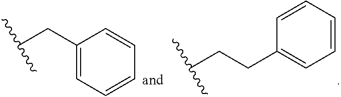 [SMILES: *Cc1ccccc1 ; *CCc1ccccc1]

In some embodiments, for the compound or salt of Formula (II), (II-A), (II-B), (II-C), (II-D), (II-E), or (II-F), R¹⁸ is selected from C₃₋₁₀ carbocycle and 3- to 10-membered heterocycle, any of which are optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —NO₂, ═O, ═S, ═N(R²¹), —CN, C₁₋₆ alkyl, and C₁₋₆ haloalkyl. In some embodiments, R¹⁸ is selected from C₃₋₁₀ carbocycle and 3- to 10-membered heterocycle, any of which are optionally substituted with one or more substituents independently selected from halogen, —OR²¹, —SR²¹, —N(R²¹)₂, —C(O)R²¹, —NO₂, ═O, ═S, ═N(R²¹), —CN, C₁₋₆ alkyl, and C₁₋₆ haloalkyl. In some embodiments, R¹⁸ is selected from C₃₋₁₀ carbocycle and 3- to 10-membered heterocycle, any of which are optionally substituted with one or more substituents independently selected from halogen, —OR²¹, —N(R²¹)₂, —C(O)R²¹, —NO₂, —CN, C₁₋₆ alkyl, and C₁₋₆ haloalkyl.

In some embodiments, for the compound or salt of Formula (II), (II-A), (II-B), (II-C), (II-D), (II-E), or (II-F), R¹⁸ is selected from C₃₋₇ carbocycle and 3- to 7-membered heterocycle, any of which are optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —NO₂, ═O, ═S, ═N(R²¹), —CN, C₁₋₆ alkyl, and C₁₋₆ haloalkyl. In some embodiments, R¹⁸ is selected from C₃₋₇ carbocycle and 3- to 7-membered heterocycle, any of which are optionally substituted with one or more substituents independently selected from halogen, —OR²¹, —SR²¹, —N(R²¹)₂, —C(O)R²¹, —NO₂, ═O, ═S, ═N(R²¹), —CN, C₁₋₆ alkyl, and C₁₋₆ haloalkyl. In some embodiments, R¹⁸ is selected from C₃₋₇ carbocycle and 3- to 7-membered heterocycle, any of which are optionally substituted with one or more substituents independently selected from halogen, —OR²¹, —N(R²¹)₂, —C(O)R²¹, —NO₂, —CN, C₁₋₆ alkyl, and C₁₋₆ haloalkyl.

In some embodiments, for the compound or salt of Formula (II), (II-A), (II-B), (II-C), (II-D), (II-E), or (II-F), R¹⁸ is selected from C₃₋₆ carbocycle and 3- to 6-membered heterocycle, any of which are optionally substituted with one or more substituents selected from halogen, —OR²¹, —N(R²¹)₂, —C(O)R²¹, —C(O)N(R²¹)₂, —C(O)OR²¹, —NO₂, ═O, —CN, C₁₋₆ alkyl, and C₁₋₆ haloalkyl. In some embodiments, R¹⁸ is selected from C₃₋₆ carbocycle and 3- to 6-membered heterocycle, any of which are optionally substituted with one or more substituents selected from halogen, —OR²¹, —N(R²¹)₂, —NO₂, —CN, C₁₋₆ alkyl, and C₁₋₆ haloalkyl. In some embodiments, R¹⁸ is selected from C₃₋₆ carbocycle and 3- to 6-membered heterocycle. In some embodiments, R¹⁸ is C₃₋₆ carbocycle.

In some embodiments, for the compound or salt of Formula (II), (II-A), (II-B), (II-C), (II-D), (II-E), or (II-F), R¹⁸ is

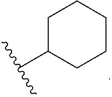 [SMILES: *C1CCCCC1]

In some embodiments, for the compound or salt of Formula (II), (II-A), (II-B), (II-C), (II-D), (II-E), or (II-F), R¹⁸ is selected from:

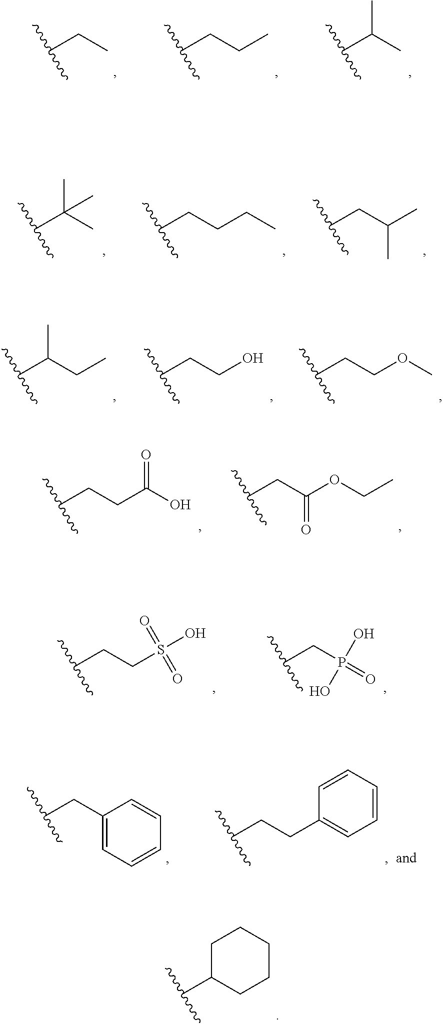

In some embodiments, for the compound or salt of Formula (II), (II-A), (II-B), (II-C), (II-D), (II-E), or (II-F),

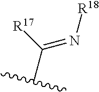

is selected from:

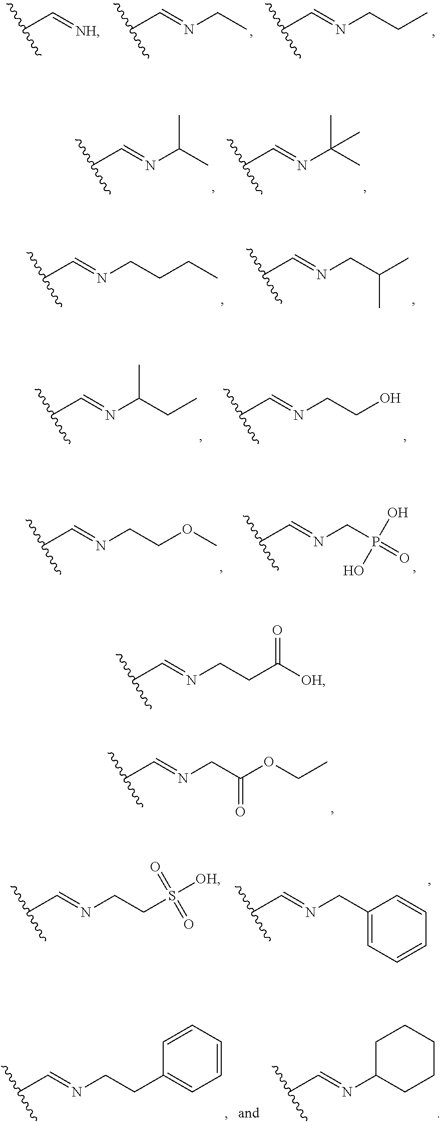

In some embodiments, for the compound or salt of Formula (I), (II), (II-A), (II-B), (II-C), (II-D), (II-E), or (II-F), wherein m is 1.

In some embodiments, for the compound or salt of Formula (I), (II), (II-A), (II-B), (II-C), (II-D), (II-E), or (II-F), wherein m is 2.

In some embodiments, for the compound or salt of Formula (I), (II), (II-A), (II-B), (II-C), (II-D), (II-E), or (II-F), R³ is selected from hydrogen and C₁₋₆ alkyl and C₁₋₆ haloalkyl. In some embodiments, wherein R³ is hydrogen.

In some embodiments, for the compound or salt of Formula (I), (II), (II-A), (II-B), (II-C), (II-D), (II-E), or (II-F), R⁴ is independently selected from halogen, —OR¹¹, —N(R¹¹)₂, —NO₂, —CN, C₁₋₆alkyl, and C₁₋₆haloalkyl. In some embodiments, R⁴ is selected from —OH, —OMe, and methyl.

In some embodiments, for the compound or salt of Formula (I), (II), (II-A), (II-B), (II-C), (II-D), (II-E), or (II-F), n is selected from 1 and 2.

In some embodiments, for the compound or salt of Formula (I), (II), (II-A), (II-B), (II-C), (II-D), (II-E), or (II-F), n is 1.

In some embodiments, for the compound or salt of Formula (I), (II), (II-A), (II-B), (II-C), (II-D), (II-E), or (II-F), n is 2.

In some embodiments, for the compound or salt of Formula (I), (II), (II-A), (II-B), (II-C), (II-D), (II-E), or (II-F), n is 3.

In some embodiments, for the compound or salt of Formula (I), (II), (II-A), (II-B), (II-C), (II-D), (II-E), or (II-F), Ring A is selected from C₃₋₈ carbocycle and 3- to 8-membered heterocycle. In some embodiments, Ring A is C₃₋₈ carbocycle. In some embodiments, Ring A is phenyl.

In some embodiments, for the compound or salt of Formula (I), (II), (II-A), or (II-D), Ring A is C₃₋₁₂ carbocycle. In some embodiments, Ring A is C₃₋₁₂ saturated carbocycle. In some embodiments, Ring A is C₃₋₁₂ unsaturated carbocycle. In some embodiments, Ring A is C₆₋₁₂ aryl. In some embodiments, Ring A is selected from C₃ carbocycle, C₄ carbocycle, C₅ carbocycle, C₆ carbocycle, C₇ carbocycle, C₈ carbocycle, C₉ carbocycle, C₁₀ carbocycle, C₁₁ carbocycle, and C₁₂ carbocycle, each of which is optionally substituted with R⁴ as defined in Formula (I). In some embodiments, Ring A is selected from C₃₋₄ carbocycle, C₃₋₅ carbocycle, C₃₋₆ carbocycle, C₃₋₇ carbocycle, C₃₋₈ carbocycle, C₃₋₉ carbocycle, C₃₋₁₀ carbocycle, C₃₋₁₁ carbocycle, and C₃₋₁₂ carbocycle, each of which is optionally substituted with R⁴ as defined in Formula (I). In some embodiments, Ring A is selected from C₃₋₈ monocyclic carbocycle and C₄₋₁₂ bicyclic carbocycle, each of which is optionally substituted with R⁴ as defined in Formula (I). In some embodiments, the C₃₋₈ monocyclic carbocycle of Ring A is selected from C₃₋₈ cycloalkyl and phenyl, each of which is optionally substituted with R⁴ as defined in Formula (I). In some embodiments, the C₄₋₁₂ bicyclic carbocycle of Ring A is selected from C₄₋₁₂ fused carbocycle, C₄₋₁₂ bridged carbocycle, and C₄₋₁₂ spirocyclic carbocycle, each of which is optionally substituted with R⁴ as defined in Formula (I).

In some embodiments, for the compound or salt of Formula (I), (II), (II-A), or (II-D), Ring A is 3- to 12-membered heterocycle. In some embodiments, Ring A is 3- to 12-membered heterocycle comprising at least one heteroatom selected from oxygen, nitrogen, and sulfur. In some embodiments, Ring A is 3- to 12-membered unsaturated heterocycle. In some embodiments, Ring A is 3- to 12-membered heteroaryl. In some embodiments, Ring A is selected from 3-membered heterocycle, 4-membered heterocycle, 5-membered heterocycle, 6-membered heterocycle, 7-membered heterocycle, 8-membered heterocycle, 9-membered heterocycle, 10-membered heterocycle, and 11-membered heterocycle, each of which is optionally substituted with R⁴ as defined in Formula (I). In some embodiments, Ring A is selected from 3- to 4-membered heterocycle, 3- to 5-membered heterocycle, 3- to 6-membered heterocycle, 3- to 7-membered heterocycle, 3- to 8-membered heterocycle, 3- to 9-membered heterocycle, 3- to 10-membered heterocycle, 3- to 11-membered heterocycle, and 3- to 12-membered heterocycle, each of which is optionally substituted with R⁴ as defined in Formula (I). In some embodiments, Ring A is selected from 3- to 8-membered monocyclic heterocycle and 4- to 12-membered bicyclic heterocycle, each of which is optionally substituted with R⁴ as defined in Formula (I). In some embodiments, Ring A is selected from 3- to 8-membered monocyclic heterocycle and 8- to 12-membered bicyclic heterocycle, each of which is optionally substituted with R⁴ as defined in Formula (I). In some embodiments, Ring A is selected from 3- to 8-membered monocyclic heterocycle and 8- to 12-membered bicyclic heterocycle. In some embodiments, the 4- to 12-membered bicyclic heterocycle is selected from 4- to 12-membered fused heterocycle, 4- to 12-membered bridged heterocycle, and 4- to 12-membered spirocyclic heterocycle, each of which is optionally substituted with R⁴ as defined in Formula (I).

In some embodiments, for the compound or salt of Formula (I), (II), (II-A), or (II-D), Ring A is selected from C₃₋₁₀ carbocycle and 3- to 10-membered heterocycle, each of which is optionally substituted with R⁴ as defined in Formula (I). In some embodiments, Ring A is C₃₋₁₀ carbocycle optionally substituted with R⁴ as defined in Formula (I). In some embodiments, Ring A is 3- to 10-membered heterocycle optionally substituted with R⁴ as defined in Formula (I).

In some embodiments, for the compound or salt of Formula (I), (II), (II-A), or (II-D), Ring A is selected from C₃₋₁₀ carbocycle and 3- to 10-membered heterocycle. In some embodiments, Ring A is C₆₋₁₀ carbocycle. In some embodiments, Ring A is a bicyclic C₆₋₁₀ carbocycle. In some embodiments, the bicyclic C₆₋₁₀ carbocycle of Ring A is selected from C₆₋₁₀ fused carbocycle, C₆₋₁₀ bridged carbocycle, and C₆₋₁₀ spirocyclic carbocycle. In some embodiments, Ring A is C₆₋₁₀ fused carbocycle. In some embodiments, Ring A is C₆₋₁₀ bridged carbocycle. In some embodiments, Ring A is C₆₋₁₀ spirocyclic carbocycle. In some embodiments, Ring A is dihydroindenyl. In some embodiments, Ring A is indanyl. In some embodiments, Ring A is

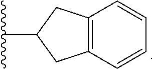 [SMILES: *C1Cc2ccccc2C1]

In some embodiments, for the compound or salt of Formula (I) or (II), wherein

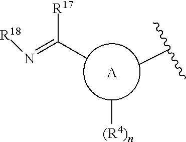

is selected from

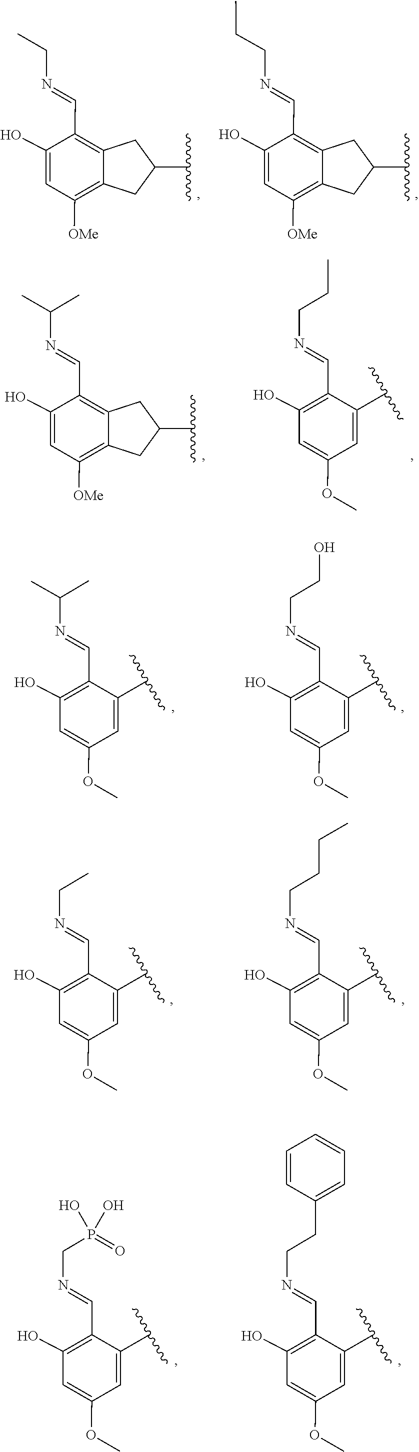

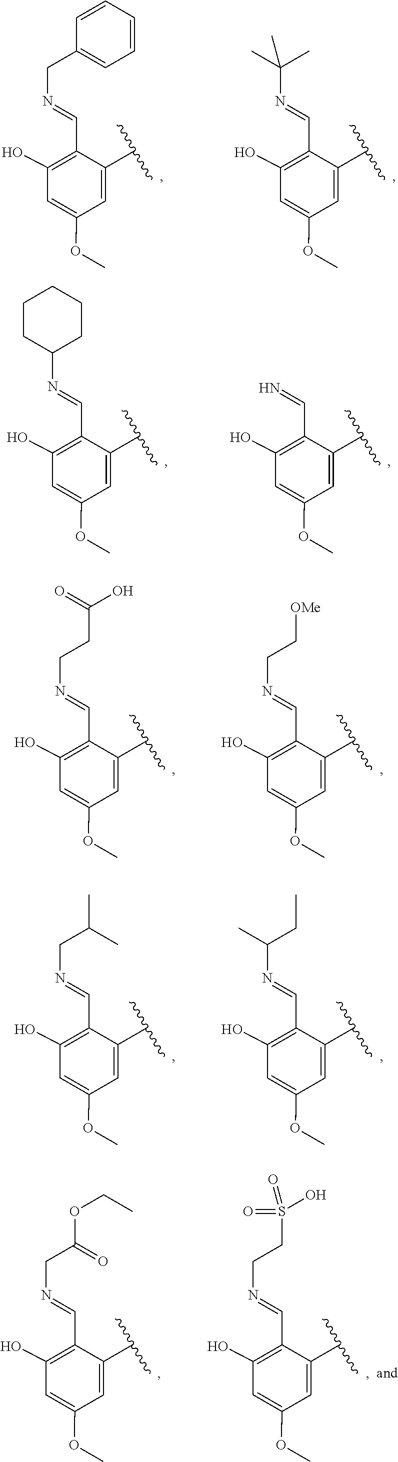

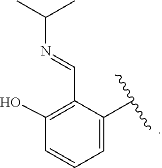 [SMILES: *c1cccc(O)c1/C=N/C(C)C]

In some embodiments, for the compound or salt of Formula (I) or (II),

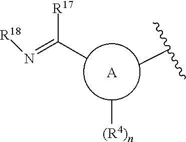

is selected from:

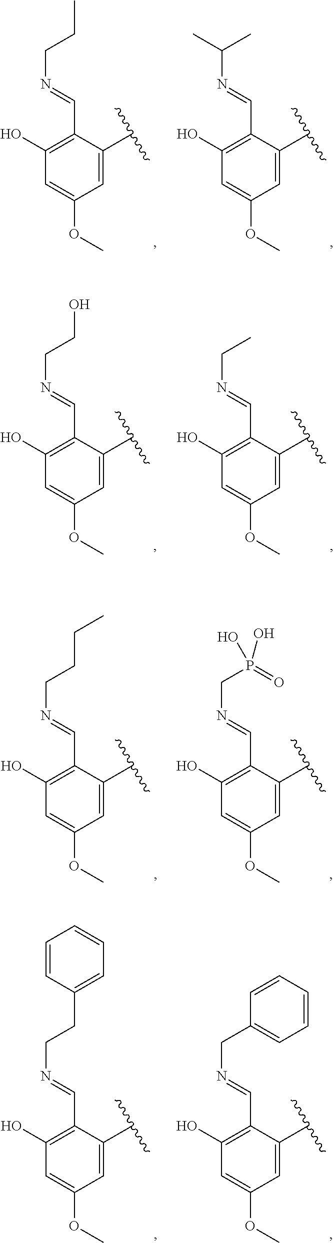

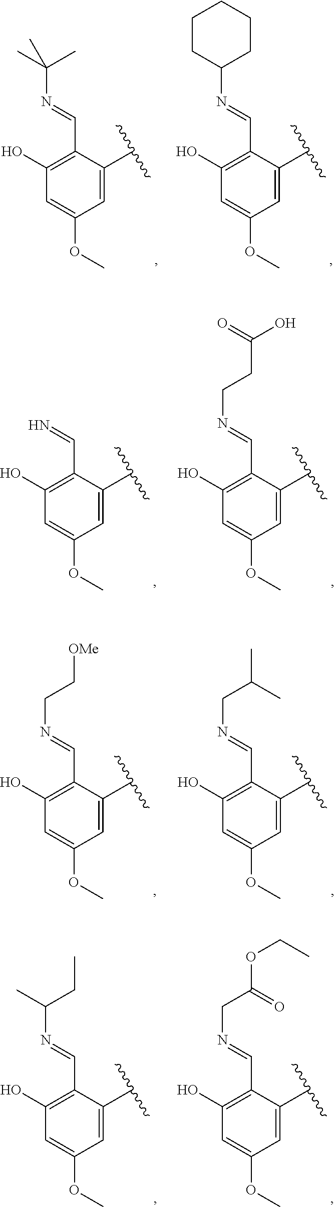

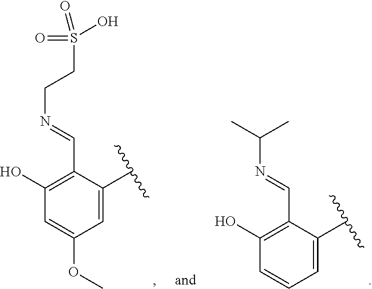 [SMILES: *c1cc(OC)cc(O)c1/C=N/CCS(=O)(=O)O ; *c1cccc(O)c1/C=N/C(C)C]

In some embodiments, for the compound or salt of Formula (I) or (II),

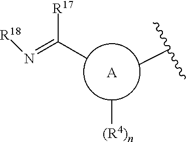

is selected from

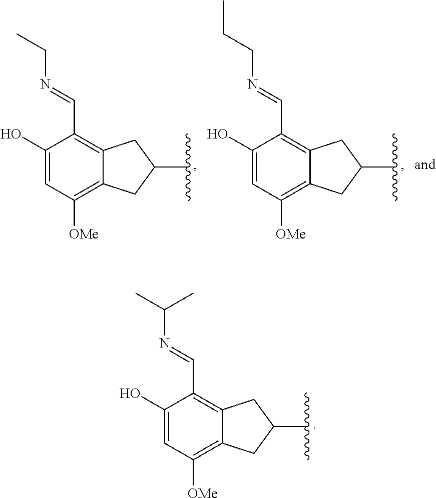 [SMILES: *C1Cc2c(OC)cc(O)c(/C=N/CC)c2C1 ; *C1Cc2c(OC)cc(O)c(/C=N/CCC)c2C1 ; *C1Cc2c(OC)cc(O)c(/C=N/C(C)C)c2C1]

In some embodiments, for the compound or salt of Formula (I), (II), (II-A), (II-B), or (II-C), one of R¹ and R² is selected from hydrogen, C₁₋₆ alkyl, and C₁₋₆ haloalkyl, and the other is selected from:
—OR¹⁴, —N(R¹⁴)₂, —C(O)R¹⁴, —C(O)N(R¹⁴)₂, —CN, C₁₋₆ alkyl, C₂₋₆ alkenyl, and C₂₋₆ alkynyl, wherein the C₁₋₆alkyl, C₂₋₆ alkenyl, and C₂₋₆ alkynyl are each optionally substituted with one or more substituents independently selected from halogen, —OR¹⁴, —N(R¹⁴)₂, —C(O)R¹⁴, —C(O)N(R¹⁴)₂, —NO₂, ═O, and —CN; and
C₃₋₁₀ carbocycle and 3- to 10-membered heterocycle, each of which are optionally substituted with halogen, —OR¹¹, —N(R¹⁴)₂, —C(O)R¹⁴, —C(O)N(R¹⁴)₂, —NO₂, ═O, —CN, C₁₋₆ alkyl, and C₁₋₆ haloalkyl.

In some embodiments, for the compound or salt of Formula (I), (II), (II-A), (II-B), or (II-C), one of R¹ and R² is selected from hydrogen, C₁₋₆ alkyl, and C₁₋₆ haloalkyl, and the other is selected from:
—OR¹⁴, —N(R¹⁴)₂, C₁₋₆ alkyl, and C₂₋₆ alkynyl, wherein the C₁₋₆alkyl and C₂₋₆ alkynyl are each optionally substituted with one or more substituents independently selected from halogen, —OR¹⁴, —N(R¹⁴)₂, and —CN; and
C₃₋₁₀ carbocycle and 3- to 10-membered heterocycle, each of which are optionally substituted with halogen, C₁₋₆ alkyl, and C₁₋₆ haloalkyl.

In some embodiments, for the compound or salt of Formula (I), (II), (II-A), (II-B), or (II-C), one of R¹ and R² is selected from hydrogen, methyl, ethyl, and propyl, and the other is selected from methyl, ethyl, propyl,

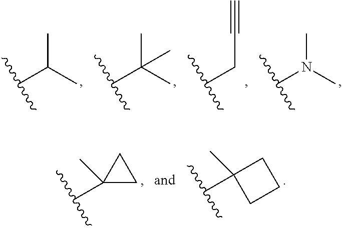

In some embodiments, for the compound or salt of Formula (I), (II), (II-A), (II-B), (II-C), (II-D), (II-E), or (II-F), R¹ and R² come together to form a 3- to 10-membered heterocycle optionally substituted with one or more substituents independently selected from halogen, —OR¹⁵, —N(R¹⁵)₂, —NO₂, ═O, —CN, C₁₋₆ alkyl, and C₁₋₆ haloalkyl.

In some embodiments, for the compound or salt of Formula (I) or (II),

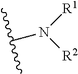

is selected from

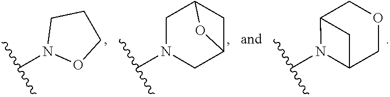

In some embodiments, for the compound or salt of Formula (I), (II), (II-A), (II-B), (II-C), (II-D), (II-E), or (II-F), any of L¹, L², L³, or L⁴ is independently selected from C₃₋₁₂ carbocyclene. In some embodiments, any of L¹, L², L³, or L⁴ is independently selected from C₃₋₁₂ unsaturated carbocyclene. In some embodiments, any of L¹, L², L³, or L⁴ is independently selected from C₃₋₁₂ arylene. In some embodiments, any of L¹, L², L³, or L⁴ is independently selected from C₃ carbocyclene, C₄ carbocyclene, C₅ carbocyclene, C₆ carbocyclene, C₇ carbocyclene, C₈ carbocyclene, C₉ carbocyclene, C₁₀ carbocyclene, and C₁₁ carbocyclene. In some embodiments, any of L¹, L², L³, or L⁴ is independently selected from C₃₋₄ carbocyclene, C₃₋₅ carbocyclene, C₃₋₆ carbocyclene, C₃₋₇ carbocyclene, C₃₋₈ carbocyclene, C₃₋₉ carbocyclene, C₃₋₁₀ carbocyclene, C₃₋₁₁ carbocyclene, and C₃₋₁₂ carbocyclene. In some embodiments, any of L¹, L², L³, or L⁴ is independently selected C₃₋₈ monocyclic carbocyclene and C₈₋₁₂ bicyclic carbocyclene. In some embodiments, any of L¹, L², L³, or L⁴ is independently selected from C₃₋₈ monocyclic carbocyclene and C₄₋₁₂ bicyclic carbocyclene. In some embodiments, any of L¹, L², L³, or L⁴ is independently selected from C₃₋₈ monocyclic carbocyclene and C₈₋₁₂ bicyclic carbocyclene. In some embodiments, the C₄₋₁₂ carbocyclene is selected from C₄₋₁₂ carbocyclene, C₄₋₁₂ bridged carbocyclene, and C₄₋₁₂ spirocyclic carbocyclene.

In some embodiments, for the compound or salt of Formula (I), (II), (II-A), (II-B), (II-C), (II-D), (II-E), or (II-F), any of L¹, L², L³, or L⁴ is independently selected from 3- to 12-membered heterocyclene. In some embodiments, any of L¹, L², L³, or L⁴ is independently selected from 3- to 12-membered heterocyclene comprising at least one heteroatom selected from oxygen, nitrogen, and sulfur. In some embodiments, any of L¹, L², L³, or L⁴ is independently selected from 3- to 12-membered unsaturated heterocyclene. In some embodiments, any of L¹, L², L³, or L⁴ is independently selected from 3- to 12-membered heteroarylene. In some embodiments, any of L¹, L², L³, or L⁴ is independently selected from 3-membered heterocyclene, 4-membered heterocyclene, 5-membered heterocyclene, 6-membered heterocyclene, 7-membered heterocyclene, 8-membered heterocyclene, 9-membered heterocyclene, 10-membered heterocyclene, and 11-membered heterocyclene. In some embodiments, any of L¹, L², L³, or L⁴ is independently selected from 3- to 4-membered heterocyclene, 3- to 5-membered heterocyclene, 3- to 6-membered heterocyclene, 3- to 7-membered heterocyclene, 3- to 8-membered heterocyclene, 3- to 9-membered heterocyclene, 3- to 10-membered heterocyclene, 3- to 11-membered heterocyclene, and 3- to 12-membered heterocyclene. In some embodiments, any of L¹, L², L³, or L⁴ is independently selected from 3- to 8-membered monocyclic heterocyclene and 4- to 12-membered bicyclic heterocyclene. In some embodiments, any of L¹, L², L³, or L⁴ is independently selected from 3- to 8-membered monocyclic heterocyclene and 8- to 12-membered bicyclic heterocyclene. In some embodiments, any of L¹, L², L³, or L⁴ is independently selected from 3- to 8-membered monocyclic heterocyclene and 8- to 12-membered bicyclic heterocyclene. In some embodiments, the 4- to 12-membered bicyclic heterocyclene is selected from 4- to 12-membered fused heterocyclene, 4- to 12-membered bridged heterocyclene, and 4- to 12-membered spirocyclic heterocyclene.

In some embodiments, for the compound or salt of Formula (I), (II), (II-A), (II-B), (II-C), (II-D), (II-E), or (II-F), each of L², L³ and L⁴ are absent and L¹ is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, and C₂₋₆ alkynylene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some embodiments, for the compound or salt of Formula (I), (II), (II-A), (II-B), (II-C), (II-D), (II-E), or (II-F), each of L², L³ and L⁴ are absent and L¹ is selected from (a) and (b): (a) —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b):(a) —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from (a) and (b): (a) —N(R¹²)C(O)—, —N(R¹²)C(O)C(O)N(R¹²)—, and —(R¹²)NC(O)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from —N(R¹²)C(O)—, —N(R¹²)C(O)C(O)N(R¹²)—, and —(R¹²)NC(O)N(R¹²)—; and R¹² is selected from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L², L³ and L⁴ are absent and L¹ is selected from (a) and (b): (a) —N(R¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b):(a) —N(R¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from (a) and (b): (a) —N(R¹²)—, —N(R¹²)C(O)—, —N(R¹²)C(O)C(O)N(R¹²)—, and —(R¹²)NC(O)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from —N(R¹²)—, —N(R¹²)C(O)—, —N(R¹²)C(O)C(O)N(R¹²)—, and —(R¹²)NC(O)N(R¹²)—; and R¹² is selected from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), L¹ is selected from —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and R¹² is independently selected at each occurrence from hydrogen, C₁₋₆ alkyl, and C₁₋₆ haloalkyl. In some embodiments, L¹ is selected from —N(R¹²)C(O)—, —N(R¹²)C(O)C(O)N(R¹²)—, and —(R¹²)NC(O)N(R¹²)—; and R¹² is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), L¹ is selected from —N(R¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and R¹² is independently selected at each occurrence from hydrogen, C₁₋₆ alkyl, and C₁₋₆ haloalkyl. In some embodiments, L¹ is selected from —N(R¹²)—, —N(R¹²)C(O)—, —N(R¹²)C(O)C(O)N(R¹²)—, and —(R¹²)NC(O)N(R¹²)—; and R¹² is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L³ and L⁴ are absent and each of L¹ and L² is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, and C₂₋₆ alkynylene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L³ and L⁴ are absent and each of L¹ and L² is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —O—, —N(R¹²)—, —N(R¹²)C(O)—, —N(R¹²)C(O)O—, —N(R¹²)S(O)₂—, —N(R¹²)S(O)₂N(R¹²)—, and —(R¹²)NC(O)N(R¹²)—; and (b) C₁₋₆ alkylene, C₂₋₆ alkenylene, C₃₋₁₀ carbocyclene, and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN. In some embodiments, each of L³ and L⁴ are absent and each of L¹ and L² are independently selected from (a) and (b): (a) —O—, —N(R¹²)—, —N(R¹²)C(O)—, and —N(R¹²)C(O)O—; and (b) C₁₋₆ alkylene and C₂₋₆ alkenylene, each of which is optionally substituted with one or more substituents independently selected from halogen, —OR¹³, and —CN. In some embodiments, each of L³ and L⁴ are absent and each of L¹ and L² are independently selected from (a) and (b): (a) —O—, —N(R¹²)—, —N(R¹²)C(O)—, and —N(R¹²)C(O)O—; and (b) C₁₋₆ alkylene and C₂₋₆ alkenylene, each of which is optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₃₋₁₀ carbocyclene and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) C₃₋₁₀ carbocyclene and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₃₋₁₀ carbocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) C₃₋₁₀ carbocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) C₃₋₆ saturated carbocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) 3- to 10-membered heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) 3- to 10-membered heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) 3- to 6-membered saturated heterocyclene and 3- to 6-membered unsaturated heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) 5- to 6-membered heteroarylene and 6- to 10-membered bicyclic heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), or (II-F), L⁴ is absent and each of L¹, L², and L³ are independently selected from (a) and (b):
(a) —O—, —N(R¹²)—, —S—, —N(R¹²)C(O)—, and —N(R¹²)C(O)O—; and
(b) C₁₋₆ alkylene optionally substituted with one or more substituents independently selected from halogen, —OR¹³, —N(R¹³)₂, and —CN.

In some embodiments, for the compound or salt of Formula (I), (II), (II-A), (II-B), (II-C), (II-D), (II-E), or (II-F), L is selected from

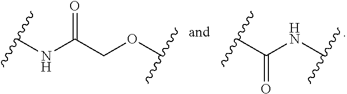 [SMILES: *NC(=O)CO* ; *NC(*)=O]

In some embodiments, for the compound or salt of Formula (I), (II), (II-A), (II-B), (II-C), (II-D), (II-E), or (II-F), L is

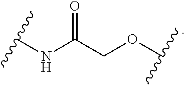 [SMILES: *NC(=O)CO*]

In some embodiments, for the compound or salt of Formula (I), the electrophilic moiety or prodrug thereof is an aldehyde or a prodrug thereof.

In some embodiments, for the compound or salt of Formula (I), the aldehyde or a prodrug thereof is an aldehyde.

In some embodiments, for the compound or salt of Formula (I), R⁵ is an aldehyde. In some embodiments, R⁵ is —C(O)H.

In some embodiments, the compound or salt of Formula (I) is represented by the structure of Formula (III):

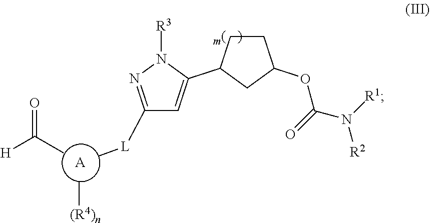

or a pharmaceutically acceptable salt thereof wherein:
Ring A is selected from C₃₋₁₂ carbocycle and 3- to 12-membered heterocycle,
each R⁴ is independently selected at each occurrence from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
L is represented by -L¹-L²-L³-L⁴-, wherein L¹, L², L³, and L⁴ are each independently selected from (a) and (b):
   (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
   (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN;
   wherein L², L³, and L⁴ are each optionally absent;
   wherein no more than two of L¹, L², L³, and L⁴ are selected from (a) and the two selected are not adjacent;
R¹ and R² are each independently selected from (i), (ii), and (iii):
   (i) hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN;
   (ii) C₁₋₆ alkyl, C₂₋₆ alkenyl, and C₂₋₆ alkynyl, each of which is optionally substituted with one or more substituents independently selected from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
      C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
   (iii) C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; or
R¹ and R² may come together to form a 3- to 10-membered heterocycle optionally substituted with one or more substituents independently selected from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN; and
   C₁₋₆ alkyl, C₂₋₆ alkenyl, and C₂₋₆ alkynyl, wherein the C₁₋₆ alkyl, C₂₋₆ alkenyl, and C₂₋₆ alkynyl are optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN;
R³ is selected from:
   hydrogen, halogen, —OR¹⁶, —SR¹⁶, —N(R¹⁶)₂, —C(O)R¹⁶, —C(O)N(R¹⁶)₂, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —C(O)OR¹⁶, —NO₂, and —CN;
R¹¹, R¹², R¹³, R¹⁴, R¹⁵, and R¹⁶ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 0, 1, 2, 3, and 4; and
m is selected from 0 and 1.

In some embodiments, for the compound or salt of Formula (I), (II), (II-A), (II-B), (II-C), (II-D), or (III), R¹ and R² are each independently selected from hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN; and C₁₋₆ alkyl, C₂₋₆ alkenyl, and C₂₋₆ alkynyl, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN; and C₁₋₆ alkyl, C₂₋₆ alkenyl, and C₂₋₆ alkynyl. In some embodiments, R¹ and R² are each independently selected from hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, —CN, and C₁₋₆ alkyl.

In some embodiments, for the compound or salt of Formula (I), (II), (II-A), (II-B), (II-C), (II-D), or (III), R¹ and R² are each independently selected from R¹ and R² are each independently selected from hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN; and R¹⁴ is independently selected at each occurrence from hydrogen, C₁₋₆ alkyl, and C₁₋₆ haloalkyl. In some embodiments, R¹ and R² are each independently selected from R¹ and R² are each independently selected from hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN. In some embodiments, for the compound or salt of Formula (I), (II), or (III), R¹ and R² are each independently selected from hydrogen, —OR¹⁴, —N(R¹⁴)₂, —C(O)R¹⁴, —C(O)N(R¹⁴)₂, and —CN. In some embodiments, R¹ and R² are each independently selected from hydrogen, —OR¹⁴, and —N(R¹⁴)₂; and each R¹⁴ is independently selected from hydrogen, C₁₋₆ alkyl, and C₁₋₆ haloalkyl. In some embodiments, R¹ and R² are each independently selected from —OR¹⁴ and —N(R¹⁴)₂; and each R¹⁴ is independently selected from hydrogen, C₁₋₃ alkyl, and C₁₋₃ haloalkyl. In some embodiments, R¹ and R² are each independently selected from —OH, —OMe, —NMe₂, and

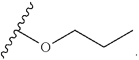 [SMILES: *OCCC]

In some embodiments, R¹ and R² are each independently selected from hydrogen, —OH, —OMe, —NMe₂, and

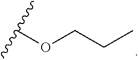 [SMILES: *OCCC]

In some embodiments, R¹ and R² are each independently selected from hydrogen, —OH, —OMe, —NMe₂,

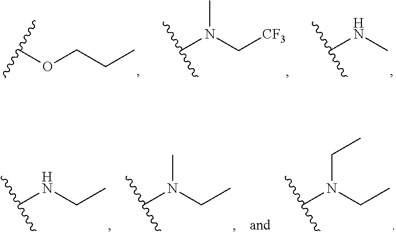

In some embodiments, R¹ and R² are each independently selected from

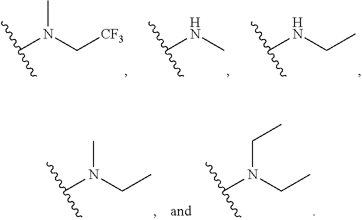

In some embodiments, for the compound or salt of Formula (I), (II), (II-A), (II-B), (II-C), (II-D), or (III), R¹ and R² are each independently selected from C₁₋₆ alkyl, C₂₋₆ alkenyl, and C₂₋₆ alkynyl, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from C₁₋₆ alkyl, C₂₋₆ alkenyl, and C₂₋₆ alkynyl, each of which is optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN.

In some embodiments, for the compound or salt of Formula (I), (II), (II-A), (II-B), (II-C), (II-D), or (III), one of R¹ and R² is selected from hydrogen and C₁₋₆ alkyl and the other is selected from C₁₋₆ alkyl, C₂₋₆ alkenyl, and C₂₋₆ alkynyl, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl and the other is selected from C₁₋₆ alkyl, C₂₋₆ alkenyl, and C₂₋₆ alkynyl, each of which is optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl and the other is selected from C₂₋₆ alkynyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl and the other is selected from C₂₋₆ alkynyl optionally substituted with one or more substituents independently selected from halogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)R¹⁴, —NO₂, ═O, and —CN. In some embodiments, one of R¹ and R² is selected from hydrogen and C₁₋₆ alkyl and the other is selected from C₂₋₆ alkynyl optionally substituted with one or more substituents independently selected from halogen, —OR¹⁴, —N(R¹⁴)₂, —NO₂, and —CN. In some embodiments, one of R¹ and R² is selected from hydrogen and C₁₋₆ alkyl and the other is selected from C₂₋₆ alkynyl. In some embodiments, one of R¹ and R² is selected from hydrogen and C₁₋₆ alkyl and the other is selected from

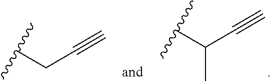

In some embodiments, for the compound or salt of Formula (I), (II), (II-A), (II-B), (II-C), (II-D), or (III), R¹ and R² are each independently selected from C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from C₃₋₁₀ carbocycle and 3- to 10-membered heterocycle, each of which is optionally substituted with one or more substituents independently selected from: halogen, —OR¹⁴, —N(R¹⁴)₂, —C(O)R¹⁴, —C(O)N(R¹⁴)₂, and —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from C₃₋₁₀ carbocycle and 3- to 10-membered heterocycle, each of which is optionally substituted with one or more substituents independently selected from: halogen, —OR¹⁴, —N(R¹⁴)₂, and —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from C₃₋₁₀ carbocycle and 3- to 10-membered heterocycle, each of which is optionally substituted with one or more substituents independently selected from and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from C₃₋₁₀ carbocycle optionally substituted with one or more substituents independently selected from and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, R¹ and R² are each independently selected from 3- to 10-membered heterocycle optionally substituted with one or more substituents independently selected from and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN.

In some embodiments, for the compound or salt of Formula (I), (II), (II-A), (II-B), (II-C), (II-D), or (III), one of R¹ and R² is selected from hydrogen and C₁₋₆ alkyl, and the other is selected from C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl, and the other is selected from C₃₋₁₀ carbocycle and 3- to 10-membered heterocycle, each of which is optionally substituted with one or more substituents independently selected from: halogen, —OR¹⁴, —N(R¹⁴)₂, —C(O)R¹⁴, —C(O)N(R¹⁴)₂, and —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl, and the other is selected from C₃₋₁₀ carbocycle and 3- to 10-membered heterocycle, each of which is optionally substituted with one or more substituents independently selected from and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN.

In some embodiments, for the compound or salt of Formula (I), (II), (II-A), (II-B), (II-C), (II-D), or (III), one of R¹ and R² is selected from hydrogen and C₁₋₆ alkyl, and the other is C₃₋₁₀ carbocycle optionally substituted with one or more substituents independently selected from and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl, and the other is C₃₋₁₀ carbocycle optionally substituted with C₂₋₆ alkynyl, the C₂₋₆ alkynyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ and R² is selected from hydrogen and C₁₋₆ alkyl, and the other is C₃₋₁₀ carbocycle optionally substituted with C₂₋₆ alkynyl, the C₂₋₆ alkynyl optionally substituted with one or more substituents independently selected from: halogen, —OR¹⁴, —N(R¹⁴)₂, —C(O)R¹⁴, —C(O)OR¹⁴, —NO₂, ═O, and —CN; and R¹⁴ is independently selected at each occurrence from hydrogen, C₁₋₆ alkyl, and C₁₋₆ haloalkyl. In some embodiments, one of R¹ and R² is selected from hydrogen and C₁₋₆ alkyl, and the other is C₃₋₁₀ carbocycle substituted with C₂₋₆ alkynyl, the C₂₋₆ alkynyl optionally substituted with one or more substituents independently selected from halogen, —OR¹⁴, —N(R¹⁴)₂, —C(O)R¹⁴, —NO₂, and —CN. In some embodiments, one of R¹ and R² is selected from hydrogen and C₁₋₆ alkyl, and the other is

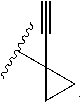 [SMILES: *C1(C#C)CC1]

In some embodiments, for the compound or salt of Formula (I), (II), (II-A), (II-B), (II-C), (II-D), or (III), one of R¹ and R² is selected from hydrogen and C₁₋₆ alkyl, and the other is selected from hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN. In some embodiments, one of R¹ and R² is selected from hydrogen and C₁₋₆ alkyl, and the other is selected from hydrogen, —OR¹⁴, —N(R¹⁴)₂, —C(O)R¹⁴, —C(O)N(R¹⁴)₂, —N(R¹⁴)C(O)OR¹⁴, N(R¹⁴)C(O)R¹⁴, and —CN. In some embodiments, one of R¹ and R² is selected from hydrogen and C₁₋₆ alkyl, and the other is selected from —OR¹⁴, —N(R¹⁴)₂, —C(O)R¹⁴, —C(O)N(R¹⁴)₂, and —CN. In some embodiments, one of R¹ and R² is selected from hydrogen and C₁₋₆ alkyl, and the other is selected from —N(R¹⁴)₂. In some embodiments, one of R¹ and R² is selected from hydrogen and C₁₋₆ alkyl, and the other is selected from NMe₂,

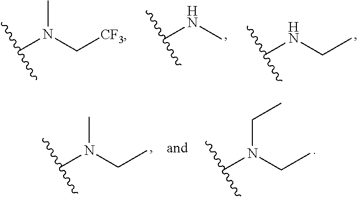

In some embodiments, one of R¹ and R² is selected from NMe₂,

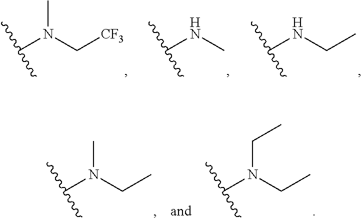

In some embodiments, for the compound or salt of Formula (I), (II), (II-A), (II-B), (II-C), (II-D), or (III), R¹ and R² may come together to form a 3- to 10-membered heterocycle optionally substituted with one or more substituents independently selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl are optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN. In some embodiments, R¹ and R² may come together to form a 3- to 10-membered heterocycle optionally substituted with one or more substituents independently selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl are optionally substituted with one or more substituents independently selected from halogen, —OR¹⁵, —N(R¹⁵)₂, —NO₂, ═O, and —CN. In some embodiments, R¹ and R² may come together to form a 3- to 10-membered heterocycle optionally substituted with one or more substituents independently selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl are optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN In some embodiments, R¹ and R² may come together to form a 3- to 10-membered heterocycle optionally substituted with C₂₋₆ alkynyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN. In some embodiments, R¹ and R² may come together to form a 3- to 10-membered heterocycle optionally substituted with C₂₋₆ alkynyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)S(O)₂R¹⁵, —S(O)₂N(R¹⁵)₂, —NO₂, ═O, and —CN In some embodiments, R¹ and R² may come together to form a 3- to 10-membered heterocycle optionally substituted with C₂₋₆ alkynyl optionally substituted with one or more substituents independently selected from halogen, —OR¹⁵, —N(R¹⁵)₂, —C(O)R¹⁵, —NO₂, and —CN. In some embodiments, R¹ and R² may come together to form a 3- to 6-membered heterocycle optionally substituted with C₂₋₆ alkynyl optionally substituted with one or more substituents independently selected from halogen, —OR¹⁵, —N(R¹⁵)₂, —C(O)R¹⁵, —NO₂, and —CN. In some embodiments,

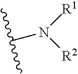

is selected from

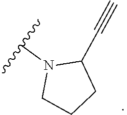 [SMILES: *N1CCCC1C#C]

In some embodiments, for the compound or salt of Formula (I), (II), (II-A), (II-B), (II-C), (II-D), or (III), R¹ and R² may come together to form a 3- to 10-membered saturated heterocycle optionally substituted with one or more substituents independently selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl are optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN. In some embodiments, R¹ and R² may come together to form a saturated 3- to 6-membered heterocycle optionally substituted with C₂₋₆ alkynyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN. In some embodiments, R¹ and R² may come together to form a saturated 3- to 6-membered heterocycle optionally substituted with C₂₋₆ alkynyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)S(O)₂R¹⁵, —S(O)₂N(R¹⁵)₂, —NO₂, ═O, and —CN In some embodiments, R¹ and R² may come together to form a pyrrolidinyl optionally substituted with C₂₋₆ alkynyl optionally substituted with one or more substituents independently selected from halogen, —OR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)S(O)₂R¹⁵, —S(O)₂N(R¹⁵)₂, —NO₂, ═O, and —CN.

In some embodiments, for the compound or salt of Formula (I), (II), (II-A), (II-B), (II-C), (II-D), or (III), R¹ and R² may come together to form a 3- to 10-membered saturated heterocycle optionally substituted with one or more C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN. In some embodiments, R¹ and R² may come together to form a 3- to 10-membered heterocycle optionally substituted C₁₋₆ alkyl optionally substituted with one or more substituents independently selected from halogen, —OR¹⁵, —N(R¹⁵)₂, —C(O)R¹⁵, —NO₂, and —CN. In some embodiments,

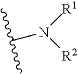

is selected from

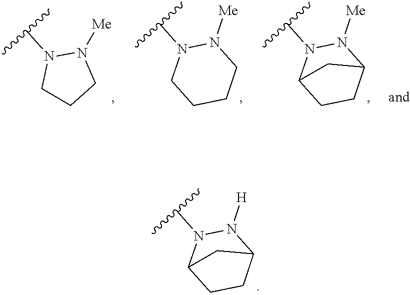 [SMILES: *N1CCCN1C ; *N1CCCCN1C ; *N1C2CCC(C2)N1C ; *N1NC2CCC1C2]

In some embodiments, for the compound or salt of Formula (I), (II), (II-A), (II-B), (II-C), (II-D), or (III),

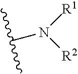

is selected from

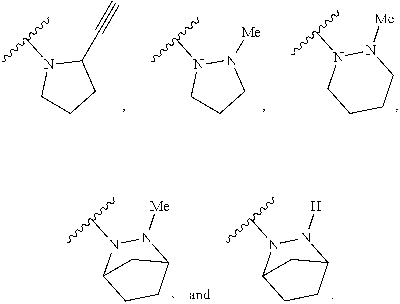

In some aspects, the compound or salt of Formula (I) or Formula (III) is represented by the structure of Formula (IV):

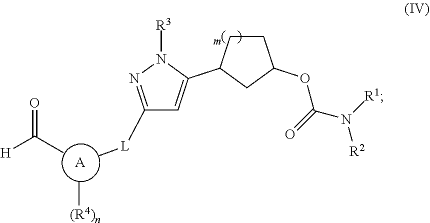

or a pharmaceutically acceptable salt thereof wherein:
Ring A is selected from C₃₋₁₂ carbocycle and 3- to 12-membered heterocycle,
each R⁴ is independently selected at each occurrence from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
   C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
L is represented by -L¹-L²-L³-L⁴-, wherein L¹, L², L³, and L⁴ are each independently selected from (a) and (b):
   (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
   (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN;
   wherein L², L³, and L⁴ are each optionally absent;
   wherein no more than two of L¹, L², L³, and L⁴ are selected from (a) and the two selected are not adjacent;
R¹ and R² are each independently selected from (i), (ii), and (iii):
   (i) hydrogen;
   (ii) C₁₋₆ alkyl optionally substituted with one or more substituents independently selected from:
      halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and
      C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
   (iii) C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; or
R¹ and R² may come together to form a 3- to 10-membered heterocycle optionally substituted with one or more substituents independently selected from:
   halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN; and
   C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN;
R³ is selected from:
   hydrogen, halogen, —OR¹⁶, —SR¹⁶, —N(R¹⁶)₂, —C(O)R¹⁶, —C(O)N(R¹⁶)₂, —C(O)OR¹⁶, —NO₂, —CN; and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —C(O)OR¹⁶, —NO₂, and —CN;
R¹¹, R¹², R¹³, R¹⁴, R, and R¹⁶ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN;
n is selected from 0, 1, 2, 3, and 4; and
m is selected from 0 and 1.

In some embodiments, m is 1.

In certain embodiments, the structure of Formula (I), (III), or (IV) is represented by Formula (IV-A):

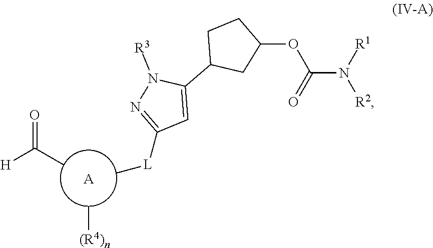

wherein, Ring A, R¹, R², R³, R⁴, L, and n are each defined as in Formula (I), (III), or (IV).

In certain embodiments, the structure of Formula (II) is represented by the structure of Formula (IV-B):

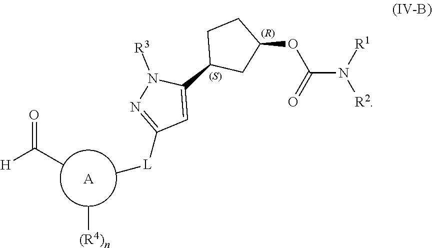

In certain embodiments, the structure of Formula (IV) is represented by the structure of Formula (IV-B):

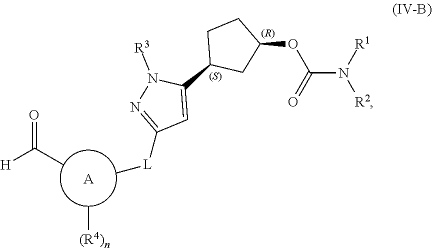

wherein R¹, R², R³, R⁴, L, Ring A, and n are each as defined as in Formula (I), (III), or (IV).

In certain embodiments, the structure of Formula (IV) is represented by the structure of Formula (IV-C):

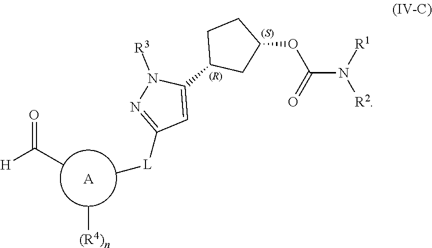

In certain embodiments, the structure of Formula (IV) is represented by the structure of Formula (IV-C):

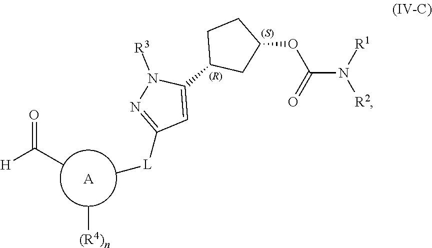

wherein R¹, R², R³, R⁴, L, Ring A, and n are each as defined as in Formula (I), (III), or (IV).

In some embodiments, m is 0. In some embodiments, m is 0 and the structure of Formula (I), (III), or (IV) is represented by Formula (V):

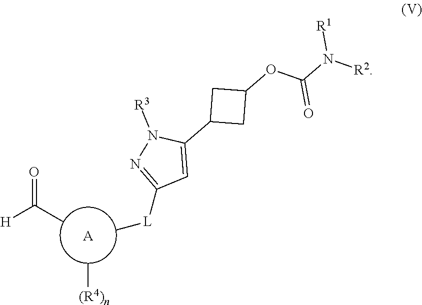

In some embodiments, m is 0 and the structure of Formula (I), (III), or (IV) is represented by Formula (V):

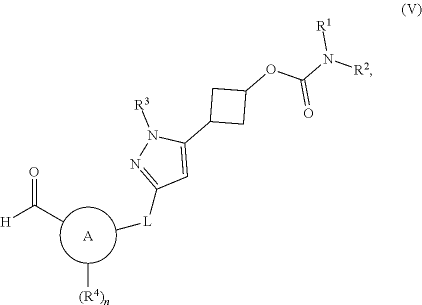

wherein, Ring A, R¹, R², R³, R⁴, L, and n are each defined as in Formula (I), (III), or (IV).

In some embodiments, for the compound or salt of Formula (I), (II), (II-A), (II-B), (II-C), (II-D), (II-E), (II-F), (III), (IV), (IV-A), (IV-B), (IV-C), or (V), n is 0.

In some embodiments, for the compound or salt of Formula (I), (II), (II-A), (II-B), (II-C), (II-D), (II-E), (II-F), (III), (IV), (IV-A), (IV-B), (IV-C), or (V), n is 1.

In some embodiments, for the compound or salt of Formula (I), (II), (II-A), (II-B), (II-C), (II-D), (II-E), (II-F), (III), (IV), (IV-A), (IV-B), (IV-C), or (V), n is 2.

In some embodiments, for the compound or salt of Formula (I), (II), (II-A), (II-B), (II-C), (II-D), (II-E), (II-F), (III), (IV), (IV-A), (IV-B), (IV-C), or (V), n is 3.

In some embodiments, for the compound or salt of Formula (I), (II), (II-A), (II-B), (II-C), (II-D), (II-E), (II-F), (III), (IV), (IV-A), (IV-B), (IV-C), or (V), n is 4.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₁₂ carbocycle. In some embodiments, Ring A is C₃₋₁₂ saturated carbocycle. In some embodiments, Ring A is C₃₋₁₂ unsaturated carbocycle. In some embodiments, Ring A is C₆₋₁₂ aryl. In some embodiments, Ring A is selected from C₃ carbocycle, C₄ carbocycle, C₅ carbocycle, C₆ carbocycle, C₇ carbocycle, C₈ carbocycle, C₉ carbocycle, C₁₀ carbocycle, C₁₁ carbocycle, and C₁₂ carbocycle, each of which is optionally substituted with R⁴ as defined in Formula (I). In some embodiments, Ring A is selected from C₃₋₄ carbocycle, C₃₋₅ carbocycle, C₃₋₆ carbocycle, C₃₋₇ carbocycle, C₃₋₈ carbocycle, C₃₋₉ carbocycle, C₃₋₁₀ carbocycle, C₃₋₁₁ carbocycle, and C₃₋₁₂ carbocycle, each of which is optionally substituted with R⁴ as defined in Formula (I). In some embodiments, Ring A is selected from C₃₋₈ monocyclic carbocycle and C₄₋₁₂ bicyclic carbocycle, each of which is optionally substituted with R⁴ as defined in Formula (I). In some embodiments, the C₃₋₈ monocyclic carbocycle of Ring A is selected from C₃₋₈ cycloalkyl and phenyl, each of which is optionally substituted with R⁴ as defined in Formula (I). In some embodiments, the C₃₋₈ monocyclic carbocycle of Ring A is phenyl. In some embodiments, the C₄₋₁₂ bicyclic carbocycle of Ring A is selected from C₄₋₁₂ fused carbocycle, C₄₋₁₂ bridged carbocycle, and C₄₋₁₂ spirocyclic carbocycle, each of which is optionally substituted with R⁴ as defined in Formula (I).

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₅₋₉ carbocycle. In some embodiments, the C₅₋₉ carbocycle of Ring A is selected from C₅₋₇ monocyclic carbocycle and C₆₋₉ bicyclic carbocycle, each of which is optionally substituted with R⁴ as defined in Formula (I). In some embodiments, the C₅₋₉ carbocycle of Ring A is C₅₋₇ monocyclic carbocycle optionally substituted with R⁴ as defined in Formula (I). In some embodiments, the C₅₋₉ carbocycle of Ring A is C₄₋₇ monocyclic carbocycle optionally substituted with R⁴ as defined in Formula (I). In some embodiments, Ring A is selected from Ring A is selected from phenyl and 2,3-dihydro-1H-indenyl, each of which is optionally substituted with R⁴ as defined in Formula (I).

In some embodiments, for the compound or salt of Formula (I), (II), (II-A), (II-B), (II-C), (II-D), (II-E), (II-F), (III), (IV), (IV-A), (IV-B), (IV-C), or (V), Ring A is selected from C₃₋₁₀ carbocycle and 3- to 10-membered heterocycle. In some embodiments, Ring A is C₆₋₁₀ carbocycle. In some embodiments, Ring A is bicyclic C₆₋₁₀ carbocycle. In some embodiments, the bicyclic C₆₋₁₀ carbocycle of Ring A is selected from C₆₋₁₀ fused carbocycle, C₆₋₁₀ bridged carbocycle, and C₆₋₁₀ spirocyclic carbocycle. In some embodiments, Ring A is C₆₋₁₀ bridged carbocycle. In some embodiments, Ring A is C₆₋₁₀ spirocyclic carbocycle. In some embodiments, Ring A is C₆₋₁₀ fused carbocycle. In some embodiments, Ring A is indanyl. In some embodiments, Ring A is is dihydroindenyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₅₋₉ carbocycle and n is 0. In some embodiments, the C₅₋₉ carbocycle of Ring A is selected from C₅₋₇ monocyclic carbocycle and C₆₋₉ bicyclic carbocycle; and n is 0.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₅₋₉ carbocycle and n is 1. In some embodiments, the C₅₋₉ carbocycle of Ring A is selected from C₅₋₇ monocyclic carbocycle and C₆₋₉ bicyclic carbocycle; and n is 1.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₅₋₉ carbocycle and n is 2. In some embodiments, the C₅₋₉ carbocycle of Ring A is selected from C₅₋₇ monocyclic carbocycle and C₆₋₉ bicyclic carbocycle; and n is 2.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₅₋₉ carbocycle and n is 3. In some embodiments, the C₅₋₉ carbocycle of Ring A is selected from C₅₋₇ monocyclic carbocycle and C₆₋₉ bicyclic carbocycle; and n is 3.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is selected from phenyl and 2,3-dihydro-1H-indenyl; n is 0. In some embodiments, Ring A is phenyl and n is 0. In some embodiments, Ring A is 2,3-dihydro-1H-indenyl and n is 0. In some embodiments,

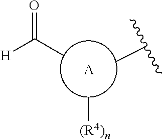

is selected from:

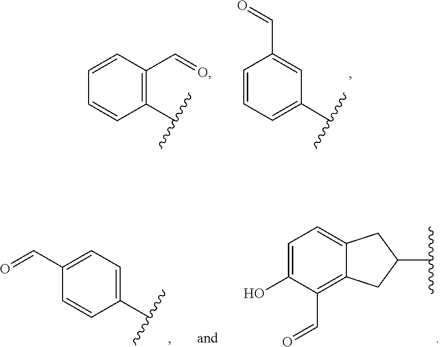

In some embodiments,

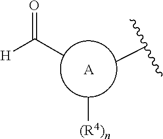

is selected from:

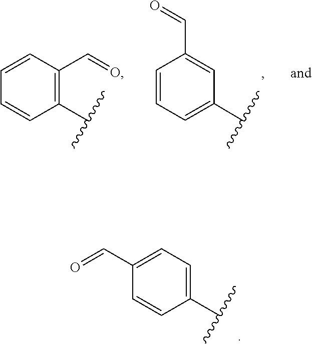

In some embodiments,

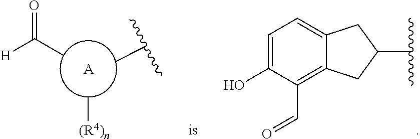

In some embodiments,

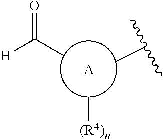

is selected from:

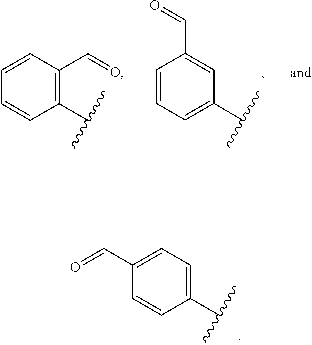

In some embodiments,

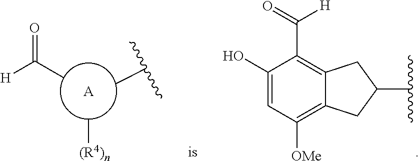

In some embodiments, wherein

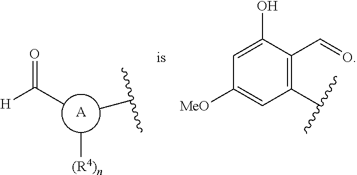

In some embodiments, for the compound or salt of Formula (I), (II), (II-A), (II-B), (II-C), (II-D), (II-E), (II-F), (III), (IV), (IV-A), (IV-B), (IV-C), or (V), R⁴ is selected from:
halogen, —OR¹¹, —SR¹¹, —N(R¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

In some embodiments, for the compound or salt of Formula (I), (II), (II-A), (II-B), (II-C), (II-D), (II-E), (II-F), (III), (IV), (IV-A), (IV-B), (IV-C), or (V), R⁴ is selected from:
halogen, —OR¹¹, —SR¹¹, —N(R¹)₂, —C(O)R¹¹, —C(O)N(R¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
5- to 6-membered heterocycle optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

In some embodiments, for the compound or salt of Formula (I), (II), (II-A), (II-B), (II-C), (II-D), (II-E), (II-F), (III), (IV), (IV-A), (IV-B), (IV-C), or (V), R⁴ is selected from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₁₋₆ alkyl optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

In some embodiments, for the compound or salt of Formula (I), (II), (II-A), (II-B), (II-C), (II-D), (II-E), (II-F), (III), (IV), (IV-A), (IV-B), (IV-C), or (V), R⁴ is selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, R⁴ is selected from halogen, —OR¹¹, —N(R¹)₂, —C(O)R¹¹, —C(O)N(R¹)₂, —N(R¹¹)C(O)R¹¹, —C(O)OR¹¹, —NO₂, ═O, ═S, ═N(R¹¹), and —CN. In some embodiments, R⁴ is selected from halogen, —OR¹¹, —N(R¹¹)₂, —C(O)R¹¹, —NO₂, and —CN. In some embodiments, R⁴ is selected from ethyl,

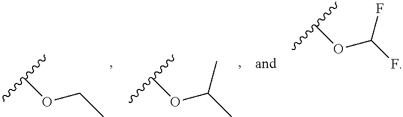

In some embodiments, for the compound or salt of Formula (I), (II), (II-A), (II-B), (II-C), (II-D), (II-E), (II-F), (III), (IV), (IV-A), (IV-B), (IV-C), or (V), R⁴ is selected from 3- to 10-membered heterocycle optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, R⁴ is selected from 3- to 6-membered heterocycle optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, R⁴ is selected from 5- to 6-membered heterocycle optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, R⁴ is selected from 5- to 6-membered heteroaryl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —NO₂, ═O, ═S, ═N(R¹¹), and —CN. In some embodiments, R⁴ is pyrazolyl optionally substituted with one or more substituents independently selected from: halogen, —OR¹¹, —N(R¹¹)₂, —C(O)R¹¹, —C(O)N(R¹¹)₂, —N(R¹¹)C(O)R¹¹, —C(O)OR¹¹, —NO₂, ═O, ═S, ═N(R¹¹), and —CN. In some embodiments, R⁴ is selected from

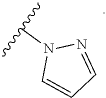 [SMILES: *n1cccn1]

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, n is 1; and R⁴ is independently selected from halogen, —OR¹¹, —N(R¹¹)₂, —C(O)N(R¹¹)₂, —N(R¹¹)C(O)R¹¹, —N(R¹¹)C(O)R¹¹, —C(O)OR¹¹, —OC(O)R¹¹, —N(R¹¹)S(O)₂R¹¹, —S(O)₂N(R¹¹)₂, —NO₂, and —CN. In some embodiments, n is 1; and R⁴ is independently selected from halogen, —OR¹¹, —N(R¹¹)₂, —C(O)N(R¹¹)₂, —N(R¹¹)C(O)R¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 1; R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 1; R⁴ is independently selected from halogen, —OR¹¹, —N(R¹¹)₂, —C(O)N(R¹¹)₂, —N(R¹¹)C(O)R¹¹, —N(R¹¹)C(O)R¹¹, —C(O)OR¹¹, —OC(O)R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 1; R⁴ is independently selected from halogen, —OR¹¹, —N(R¹¹)₂, —C(O)N(R¹¹)₂, —N(R¹¹)C(O)R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₁₂ carbocycle; n is 1; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is C₃₋₁₂ carbocycle; n is 1; and R⁴ is independently selected from halogen, —OR¹¹, —N(R¹¹)₂, —C(O)N(R¹¹)₂, —N(R¹¹)C(O)R¹¹, —N(R¹¹)C(O)R¹¹, —C(O)OR¹¹, —OC(O)R¹¹, —N(R¹¹)S(O)₂R¹¹, —S(O)₂N(R¹¹)₂, —NO₂, and —CN. In some embodiments, Ring A is C₃₋₁₂ carbocycle; n is 1; and R⁴ is independently selected from halogen, —OR¹¹, —N(R¹¹)₂, —C(O)N(R¹¹)₂, —N(R¹¹)C(O)R¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₇ carbocycle; n is 1; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is C₃₋₇ carbocycle; n is 1; and R⁴ is independently selected from halogen, —OR¹¹, —N(R¹¹)₂, —C(O)N(R¹¹)₂, —N(R¹¹)C(O)R¹¹, —N(R¹¹)C(O)R¹¹, —C(O)OR¹¹, —OC(O)R¹¹, —N(R¹¹)S(O)₂R¹¹, —S(O)₂N(R¹¹)₂, —NO₂, and —CN. In some embodiments, Ring A is C₃₋₇ carbocycle; n is 1; and R⁴ is independently selected from halogen, —OR¹¹, —N(R¹¹)₂, —C(O)N(R¹¹)₂, —N(R¹¹)C(O)R¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is phenyl; n is 1; and R⁴ is independently selected from halogen, —OR¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is phenyl; n is 1; and R⁴ is independently selected from halogen, —OR¹¹, —N(R¹¹)₂, —C(O)N(R¹¹)₂, —N(R¹¹)C(O)R¹¹, —N(R¹¹)C(O)R¹¹, —C(O)OR¹¹, —OC(O)R¹¹, —N(R¹¹)S(O)₂R¹¹, —S(O)₂N(R¹¹)₂, —NO₂, and —CN. In some embodiments, Ring A is phenyl; n is 1; and R⁴ is independently selected from halogen, —OR¹¹, —N(R¹¹)₂, —C(O)N(R¹¹)₂, —N(R¹¹)C(O)R¹¹, —N(R¹¹)S(O)₂R¹¹, —S(O)₂N(R¹¹)₂, —NO₂, and —CN.

In some embodiments for the compound or salt of Formula (I), (III), (IV), (IV-A), (IV-B), (IV-C), or (V),

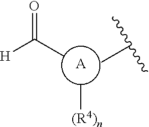

is selected from:

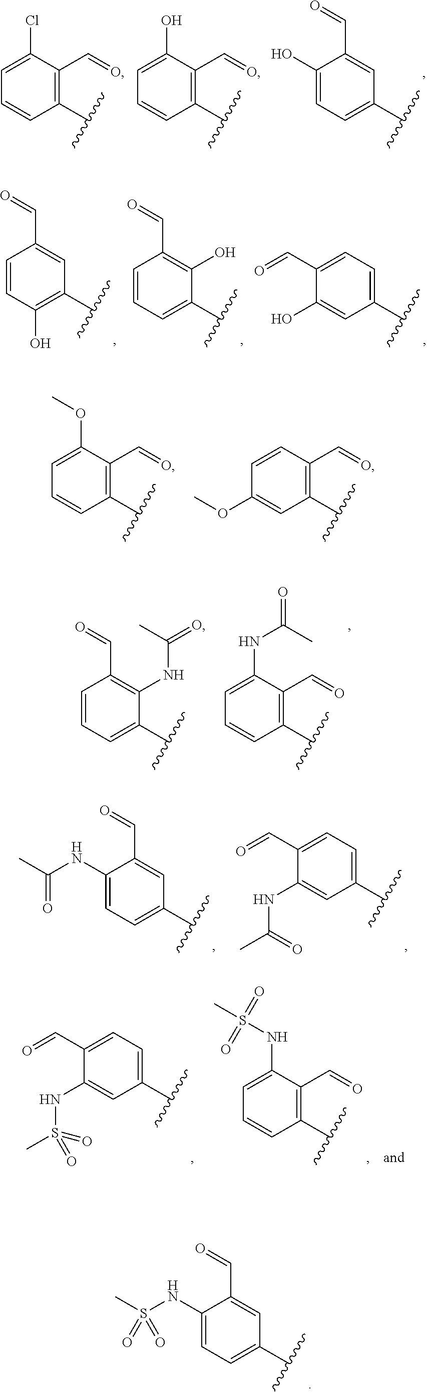

In some embodiments,

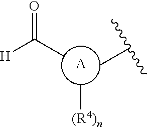

is selected from:

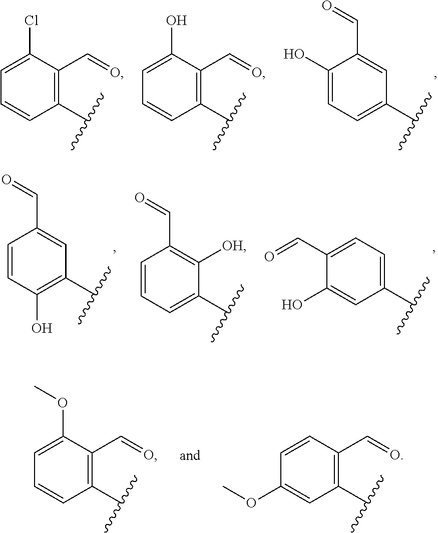

In some embodiments,

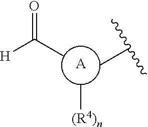

is selected from:

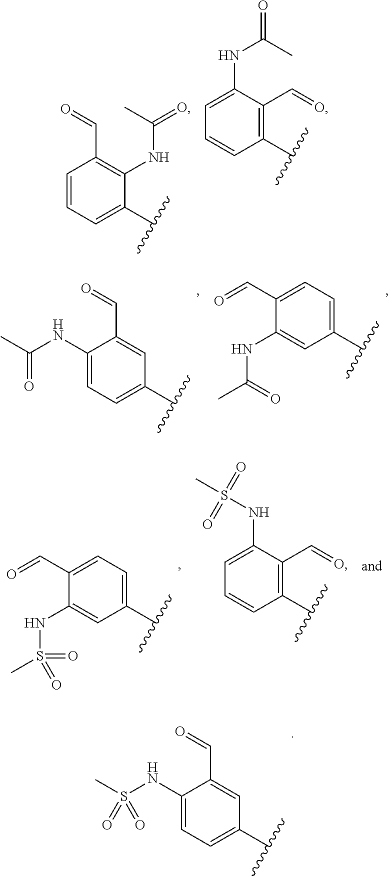

In some embodiments,

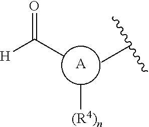

is selected from:

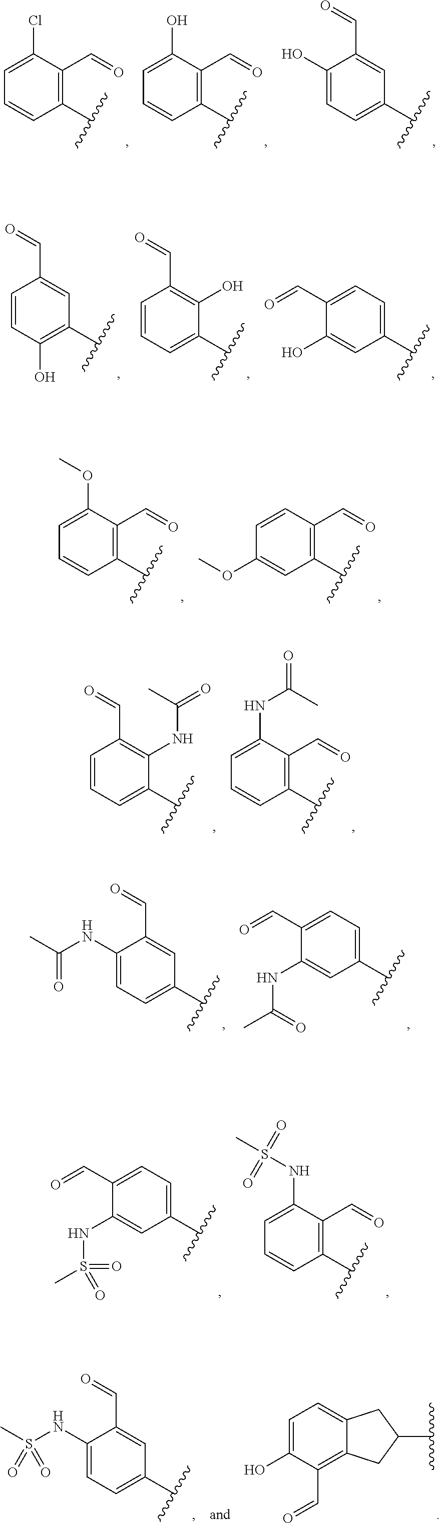

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, n is 1 and R⁴ is independently selected from C₁₋₆ alkyl and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, n is 1 and R⁴ is independently selected from C₁₋₆ alkyl and C₂₋₆ alkynyl, each of which is optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and R¹¹ is selected from hydrogen, C₁₋₆ alkyl, and C₁₋₆ haloalkyl. In some embodiments, n is 1 and R⁴ is independently selected from C₁₋₆ alkyl and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 1 and R⁴ is independently selected from C₁₋₆ alkyl and C₂₋₆ alkynyl, each of which is optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₁₂ carbocycle; n is 1; and R⁴ is independently selected from C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is C₃₋₁₂ carbocycle; n is 1; and R⁴ is independently selected from C₁₋₆ alkyl and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is C₃₋₁₂ carbocycle; n is 1; and R⁴ is independently selected from C₁₋₆ alkyl and C₂₋₆ alkynyl, each of which is optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₇ carbocycle; n is 1; and R⁴ is independently selected from C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is C₃₋₇ carbocycle; n is 1; and R⁴ is independently selected from C₁₋₆ alkyl and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is C₃₋₇ carbocycle; n is 1; and R⁴ is independently selected from C₁₋₆ alkyl and C₂₋₆ alkynyl, each of which is optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is phenyl; n is 1; and R⁴ is independently selected from C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is phenyl; n is 1; and R⁴ is independently selected from C₁₋₆ alkyl and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is phenyl; n is 1; and R⁴ is independently selected from C₁₋₆ alkyl and C₂₋₆ alkynyl, each of which is optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (III), (IV), (IV-A), (IV-B), (IV-C), or (V),

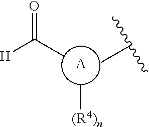

is selected from:

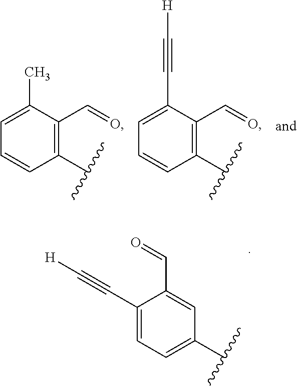

In some embodiments,

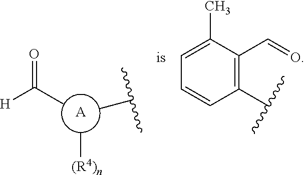

In some embodiments,

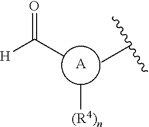

is selected from:

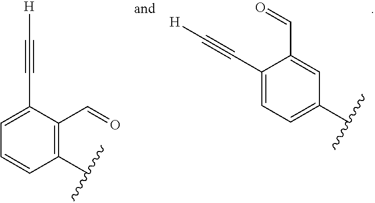 [SMILES: *c1cccc(C#C)c1C=O ; *c1ccc(C#C)c(C=O)c1]

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, n is 1 and R⁴ is independently selected from 3- to 6-membered heteroaryl optionally substituted with one or more substituents independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and R¹¹ is selected from halogen, C₁₋₆ alkyl and C₁₋₆ haloalkyl. In some embodiments, n is 1 and R⁴ is independently selected from 3- to 6-membered heteroaryl optionally substituted with one or more substituents independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from halogen, C₁₋₆ alkyl and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₁₂ carbocycle; n is 1; and R⁴ is independently selected from C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is C₃₋₁₂ carbocycle; n is 1; and R⁴ is independently selected from 3- to 6-membered heteroaryl optionally substituted with one or more substituents independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is C₃₋₇ carbocycle; n is 1; and R⁴ is independently selected from C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is C₃₋₇ carbocycle; n is 1; and R⁴ is independently selected from 3- to 6-membered heteroaryl optionally substituted with one or more substituents independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is phenyl; n is 1; and R⁴ is independently selected from C₃₋₁₀ carbocycle and 3- to 10-membered heterocycle, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is phenyl; n is 1; and R⁴ is independently selected from 3- to 6-membered heteroaryl optionally substituted with one or more substituents independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments,

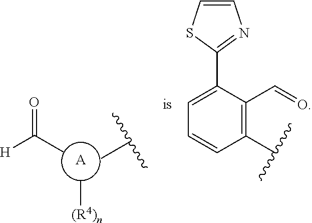

In some embodiments, for the compound or salt of Formula (I), (II), (II-A), (II-B), (II-C), (II-D), (II-E), (II-F), (III), (IV), (IV-A), (IV-B), (IV-C), or (V), n is 2 and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, n is 2 and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN. In some embodiments, n is 2 and each R⁴ is independently selected at each occurrence from: C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, n is 2 and each R⁴ is independently selected at each occurrence from: C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 2 and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and R¹¹ is selected from hydrogen, C₁₋₆ alkyl, and C₁₋₆ haloalkyl. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 2 and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 2 and each R⁴ is independently selected at each occurrence from: C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and R¹¹ is selected from hydrogen, C₁₋₆ alkyl, and C₁₋₆ haloalkyl. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 2 and each R⁴ is independently selected at each occurrence from: C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a C₃₋₁₂ carbocycle; n is 2; and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is a C₃₋₁₂ carbocycle; n is 2; and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is a C₃₋₁₂ carbocycle; n is 2; and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is a C₃₋₁₂ carbocycle; n is 2; and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is a C₃₋₁₂ carbocycle; n is 2; and each R⁴ is independently selected at each occurrence from: C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is a C₃₋₁₂ carbocycle; n is 2; and each R⁴ is independently selected at each occurrence from: C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a C₃₋₇ carbocycle; n is 2; and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is a C₃₋₇ carbocycle; n is 2; and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is a C₃₋₇ carbocycle; n is 2; and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is a C₃₋₇ carbocycle; n is 2; and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is a C₃₋₇ carbocycle; n is 2; and each R⁴ is independently selected at each occurrence from: C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is a C₃₋₇ carbocycle; n is 2; and each R⁴ is independently selected at each occurrence from: C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is phenyl; n is 2; and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is phenyl; n is 2; and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is phenyl; n is 2; and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is phenyl; n is 2; and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is phenyl; n is 2; and each R⁴ is independently selected at each occurrence from: C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, Ring A is phenyl; n is 2; and each R⁴ is independently selected at each occurrence from: C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 2 and each R⁴ is independently selected at each occurrence from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and 3- to 10-membered heterocycle optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and 3- to 10-membered heterocycle optionally substituted with one or more substituents independently selected from: halogen, —OR¹¹, —N(R¹¹)₂, —C(O)R¹¹, —N(R¹¹)C(O)R¹¹, —N(R¹¹)S(O)₂R¹¹, —S(O)₂N(R¹¹)₂, —NO₂, and —CN. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and 3- to 6-membered heterocycle optionally substituted with one or more substituents independently selected from: halogen, —OR¹¹, —N(R¹¹)₂, —C(O)R¹¹, —N(R¹¹)C(O)R¹¹, —N(R¹¹)S(O)₂R¹¹, —S(O)₂N(R¹¹)₂, —NO₂, and —CN. In some embodiments, n is 2 and each R⁴ is independently selected at each occurrence from: halogen, —OR¹¹, —N(R¹¹)₂, —C(O)R¹¹, —N(R¹¹)C(O)R¹¹, —N(R¹¹)C(O)OR¹¹, —N(R¹¹)S(O)₂R¹¹, —S(O)₂N(R¹¹)₂, —NO₂, and —CN; and 5- to 6-membered heteroaryl optionally substituted with one or more substituents independently selected from: halogen, —OR¹¹, —N(R¹¹)₂, —C(O)R¹¹, —N(R¹¹)C(O)R¹¹, —N(R¹¹)S(O)₂R¹¹, —S(O)₂N(R¹¹)₂, —NO₂, and —CN. In some embodiments, Ring A is phenyl; n is 2 and each R⁴ is independently selected at each occurrence from: halogen, —OR¹¹, —N(R¹¹)₂, and —CN; and 3- to 6-membered heterocycle. In some embodiments,

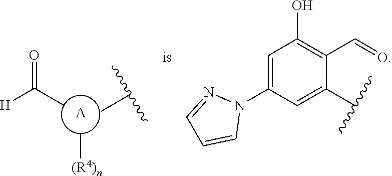

In some embodiments, for the compound or salt of Formula (I), (II), (II-A), (II-B), (II-C), (II-D), (II-E), (II-F), (III), (IV), (IV-A), (IV-B), (IV-C), or (V),

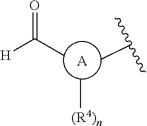

is selected from:

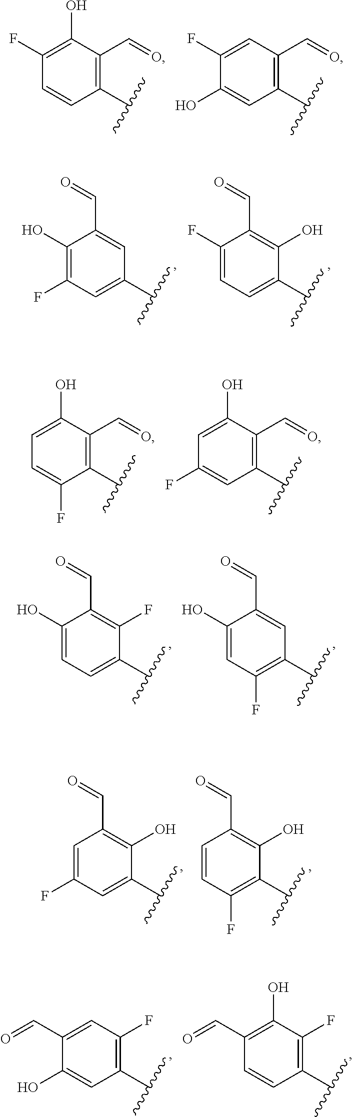

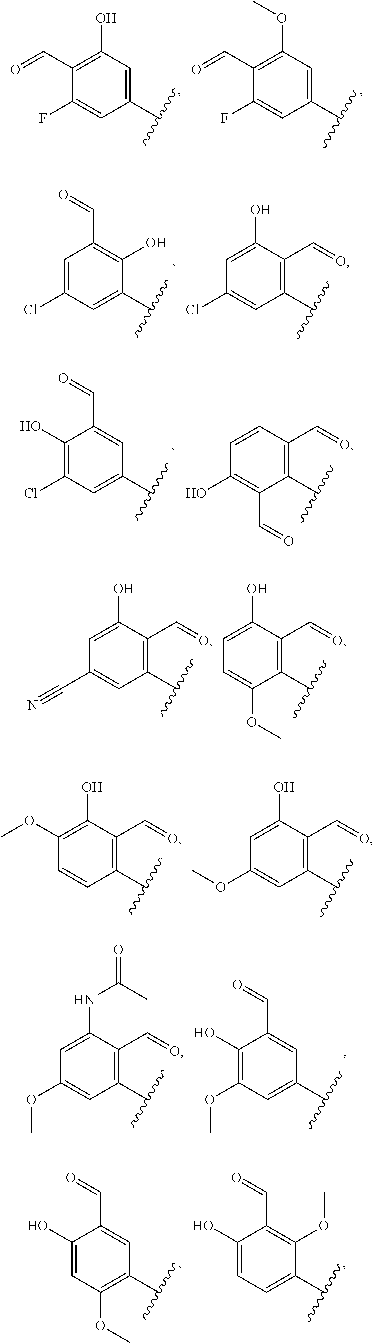

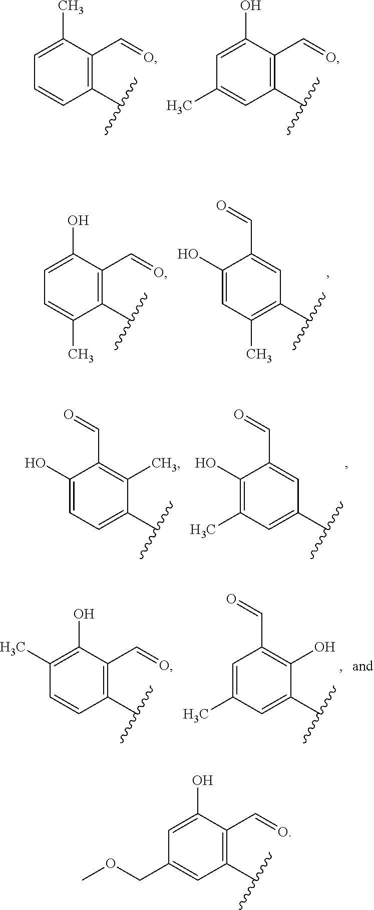

In some embodiments,

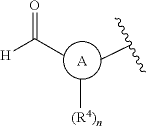

is selected from:

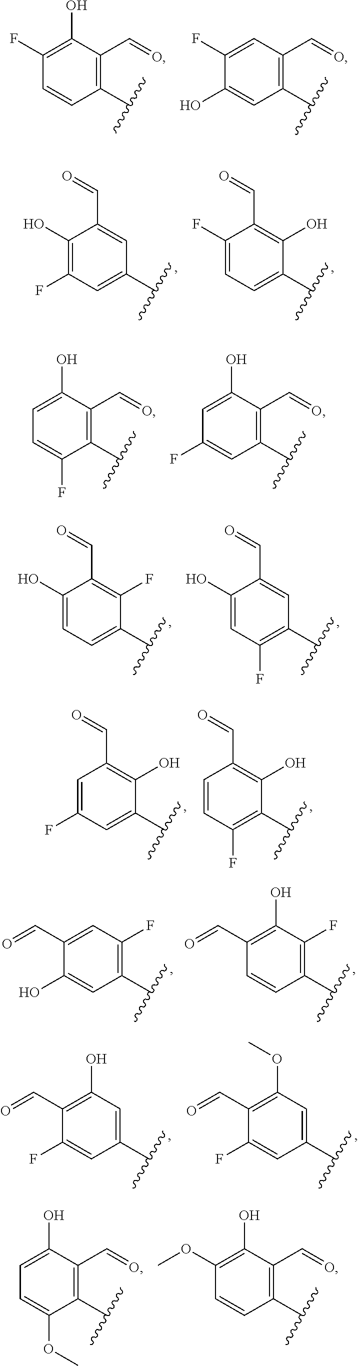

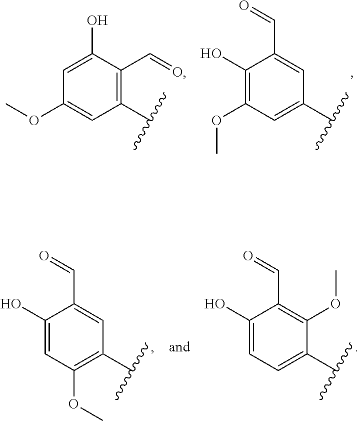

In some embodiments,

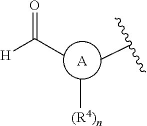

is selected from:

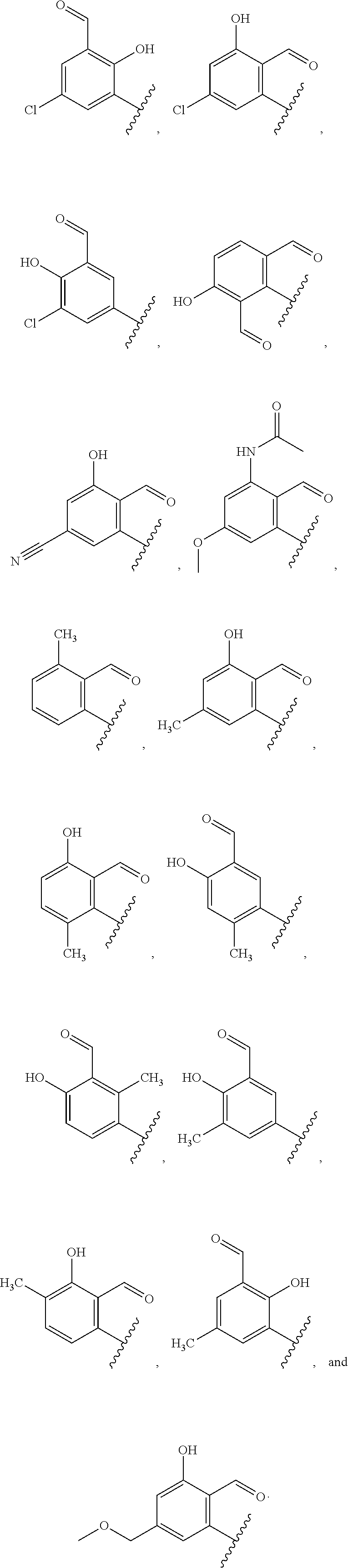

In some embodiments, for the compound or salt of Formula (I), (II), (II-A), (II-B), (II-C), (II-D) (II-E), (II-F), (III), (IV), (IV-A), (IV-B), (IV-C), or (V),

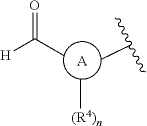

is selected from:

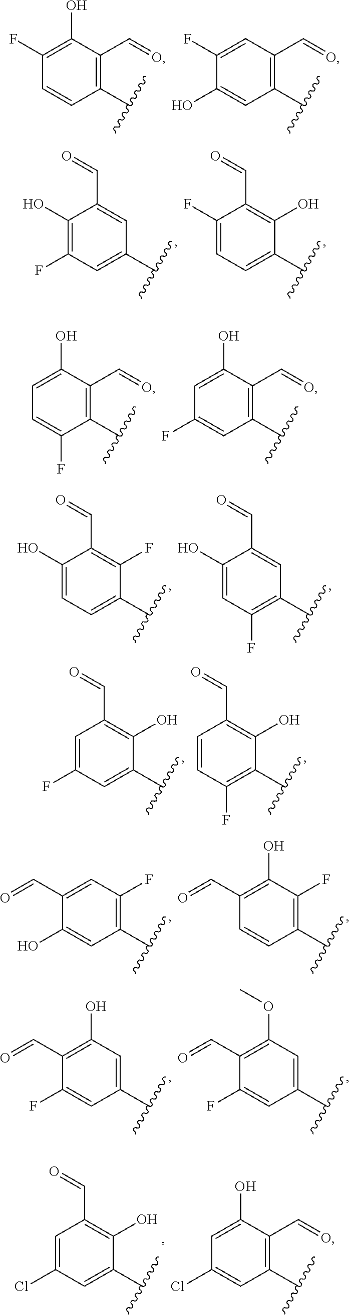

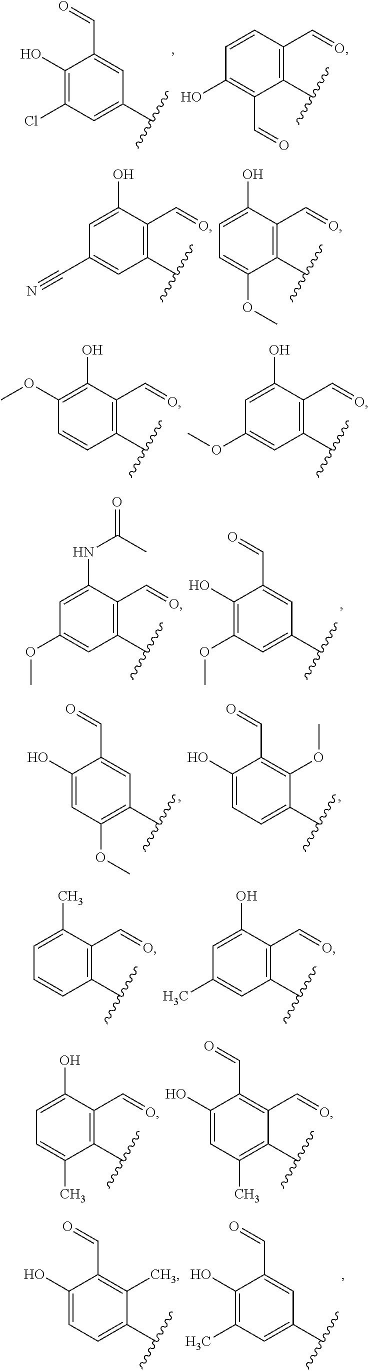

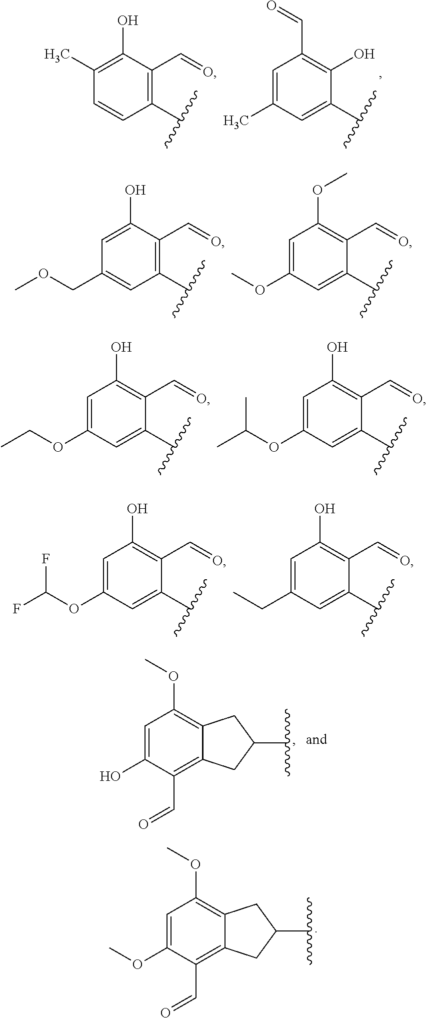

In some embodiments, for the compound or salt of Formula (I), (II), (II-A), (II-B), (II-C), (II-D), (II-E), (II-F), (III), (IV), (IV-A), (IV-B), (IV-C), or (V), n is 3 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, n is 3 and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 3 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, n is 3 and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 3 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 3 and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), n is 3 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 3 and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a C₃₋₁₂ carbocycle; n is 3; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, Ring A is a C₃₋₁₂ carbocycle; n is 3; and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a C₃₋₁₂ carbocycle; n is 3; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is a C₃₋₁₂ carbocycle; n is 3; and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a C₃₋₇ carbocycle; n is 3; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is a C₃₋₇ carbocycle; n is 3; and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is phenyl; n is 3; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is phenyl; n is 3; and R⁴ is independently selected from halogen, —OR¹¹, —N(R¹¹)₂, —C(O)R¹¹, —C(O)N(R¹¹)₂, —N(R¹¹)C(O)R¹¹, —S(O)R¹¹, —S(O)₂R¹¹, —N(R¹¹)S(O)₂R¹¹, —S(O)₂N(R¹¹)₂, —NO₂, and —CN.

In some embodiments, for the compound or salt of Formula (I), (III), (IV), (IV-A), (IV-B), (IV-C), or (V),

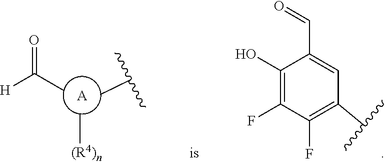

In some embodiments, for the compound or salt of Formula (I), (II), (II-A), (II-B), (II-C), (II-D), (II-E), (II-F), (III), (IV), (IV-A), (IV-B), (IV-C), or (V), Ring A is 3- to 12-membered heterocycle. In some embodiments, Ring A is 3- to 12-membered heterocycle comprising at least one heteroatom selected from oxygen, nitrogen, and sulfur. In some embodiments, Ring A is 3- to 12-membered unsaturated heterocycle. In some embodiments, Ring A is 3- to 12-membered heteroaryl. In some embodiments, Ring A is selected from 3-membered heterocycle, 4-membered heterocycle, 5-membered heterocycle, 6-membered heterocycle, 7-membered heterocycle, 8-membered heterocycle, 9-membered heterocycle, 10-membered heterocycle, and 11-membered heterocycle, each of which is optionally substituted with R⁴ as defined in Formula (I). In some embodiments, Ring A is selected from 3- to 4-membered heterocycle, 3- to 5-membered heterocycle, 3- to 6-membered heterocycle, 3- to 7-membered heterocycle, 3- to 8-membered heterocycle, 3- to 9-membered heterocycle, 3- to 10-membered heterocycle, 3- to 11-membered heterocycle, and 3- to 12-membered heterocycle, each of which is optionally substituted with R⁴ as defined in Formula (I). In some embodiments, Ring A is selected from 3- to 8-membered monocyclic heterocycle and 4- to 12-membered bicyclic heterocycle, each of which is optionally substituted with R⁴ as defined in Formula (I). In some embodiments, Ring A is selected from 3- to 8-membered monocyclic heterocycle and 8- to 12-membered bicyclic heterocycle, each of which is optionally substituted with R⁴ as defined in Formula (I). In some embodiments, Ring A is selected from 3- to 8-membered monocyclic heterocycle and 8- to 12-membered bicyclic heterocycle. In some embodiments, the 4- to 12-membered bicyclic heterocycle is selected from 4- to 12-membered fused heterocycle, 4- to 12-membered bridged heterocycle, and 4- to 12-membered spirocyclic heterocycle, each of which is optionally substituted with R⁴ as defined in Formula (I).

In some embodiments, for the compound or salt of Formula (I), (II), (II-A), (II-B), (II-C), (II-D), (II-E), (II-F), (III), (IV), (IV-A), (IV-B), (IV-C), or (V), Ring A is 3- to 12-membered heterocycle. In some embodiments, the 3- to 12-membered heterocycle is selected from 3- to 8-membered monocyclic heterocycle and 8- to 12-membered bicyclic heterocycle, each of which is optionally substituted with R⁴ as defined in Formula (I). In some embodiments, the 3- to 12-membered heterocycle is 3- to 8-membered monocyclic heterocycle, optionally substituted with R⁴ as defined in Formula (I). In some embodiments, the 3- to 12-membered heterocycle is 8- to 12-membered bicyclic heterocycle, optionally substituted with R⁴ as defined in Formula (I). In some embodiments, Ring A is selected from pyridyl, isoindolinyl, dihydrobenzofuranyl, indazolyl, benzo[d]thiazolyl, and isoquinolinyl, each of which is optionally substituted with R⁴ as defined in Formula (I).

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 12-membered heterocycle and n is 0. In some embodiments, the 3- to 12-membered of Ring A is selected from 3- to 8-membered monocyclic heterocycle and 8- to 12-membered bicyclic heterocycle; and n is 0.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 12-membered heterocycle and n is 1. In some embodiments, the 3- to 12-membered of Ring A is selected from 3- to 8-membered monocyclic heterocycle and 8- to 12-membered bicyclic heterocycle; and n is 1.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 12-membered heterocycle and n is 2. In some embodiments, the 3- to 12-membered of Ring A is selected from 3- to 8-membered monocyclic heterocycle and 8- to 12-membered bicyclic heterocycle; and n is 2.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 12-membered heterocycle and n is 3. In some embodiments, the 3- to 12-membered of Ring A is selected from 3- to 8-membered monocyclic heterocycle and 8- to 12-membered bicyclic heterocycle; and n is 3.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is selected from pyridyl, isoindolinyl, dihydrobenzofuranyl, indazolyl, benzo[d]thiazolyl, and isoquinolinyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is selected from pyridyl, isoindolinyl, dihydrobenzofuranyl, indazolyl, benzo[d]thiazolyl, and isoquinolinyl and n is 0. In some embodiments, n is 0.

In some embodiments, for the compound or salt of Formula (I), (III), (IV), (IV-A), (IV-B), (IV-C), or (V),

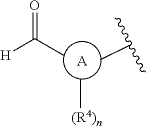

is selected from

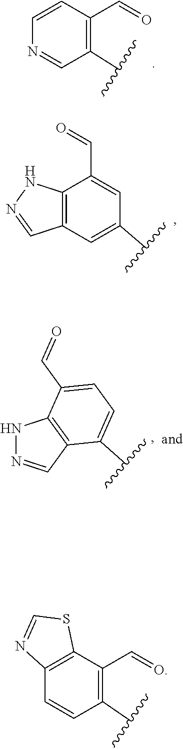 [SMILES: *c1cnccc1C=O ; *c1cc(C=O)c2[nH]ncc2c1 ; *c1ccc(C=O)c2[nH]ncc12 ; *c1ccc2ncsc2c1C=O]

In some embodiments, for the compound or salt of Formula (I), (II), (II-A), (II-B), (II-C), (II-D), (II-E), (II-F), (III), (IV), (IV-A), (IV-B), (IV-C), or (V), Ring A is selected from pyridyl, isoindolinyl, dihydrobenzofuranyl, indazolyl, benzo[d]thiazolyl, and isoquinolinyl and n is 1.

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, —CN, and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, n is 1 and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, n is 1 and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), n is 1 and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl. In some embodiments, n is 1 and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN; and R¹¹ is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 12-membered heterocycle; n is 1; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN. In some embodiments, Ring A is a 3- to 12-membered heterocycle; n is 1; and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, —CN; and C₁₋₆ alkyl optionally substituted with one or more substituents independently selected from: halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 12-membered heterocycle; n is 1; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is a 3- to 12-membered heterocycle; n is 1; and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is an 8- to 12-membered bicyclic heterocycle; n is 1; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is an 8- to 12-membered bicyclic heterocycle; n is 1; and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), Ring A is a 3- to 8-membered monocyclic heterocycle; n is 1; and R⁴ is independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, and —CN. In some embodiments, Ring A is a 3- to 8-membered monocyclic heterocycle; n is 1; and R⁴ is independently selected from halogen, —OR¹¹, —N(R¹¹)₂, —C(O)R¹¹, —N(R¹¹)C(O)OR¹¹, —N(R¹¹)S(O)₂R¹¹, —S(O)₂N(R¹¹)₂, —NO₂, and —CN.

In some embodiments, for the compound or salt of Formula (I), (III), (IV), (IV-A), (IV-B), (IV-C), or (V),

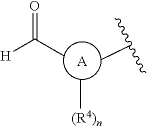

is selected from

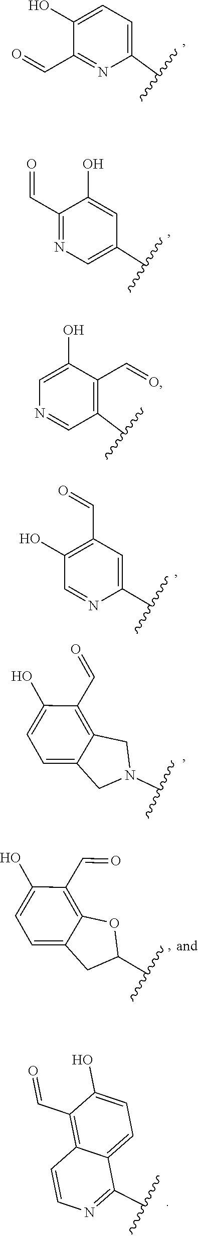

In some embodiments, for the compound or salt of Formula (I), (II), (III), (IV), (IV-A), (IV-B), (IV-C), or (V),

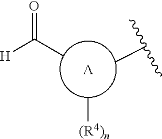

is selected from

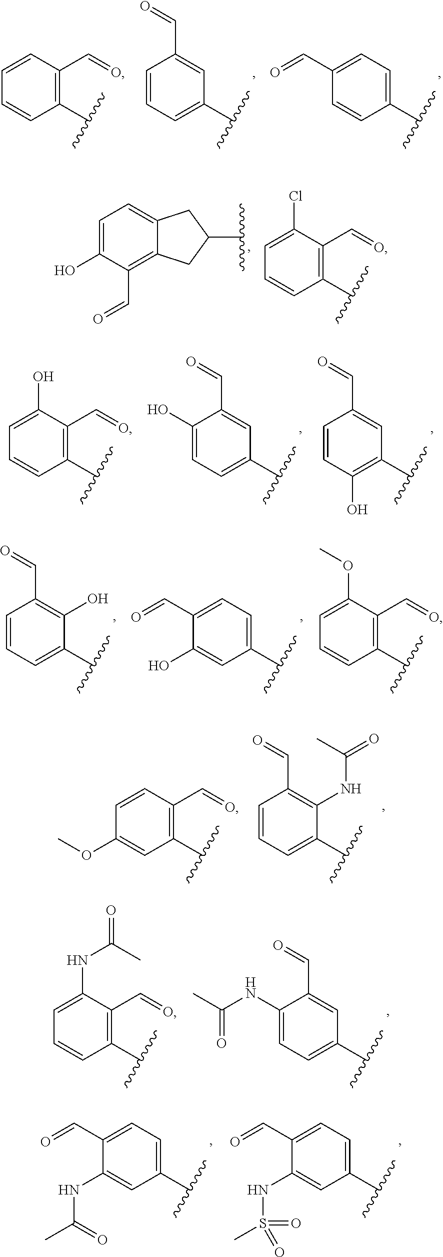

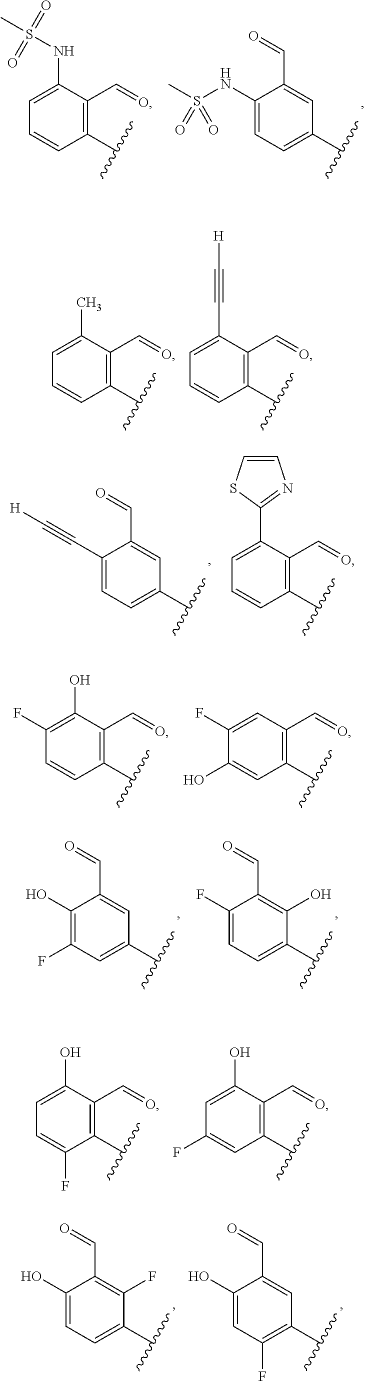

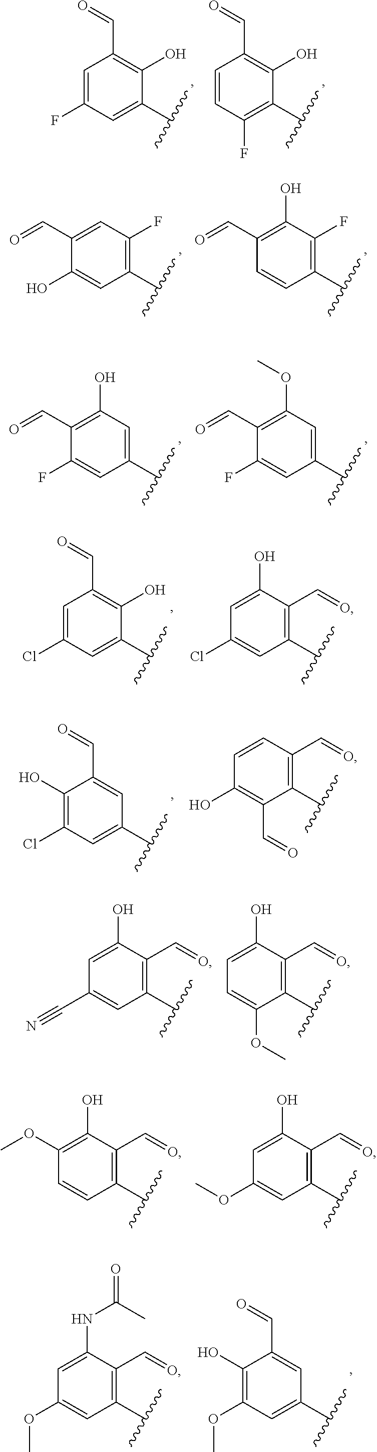

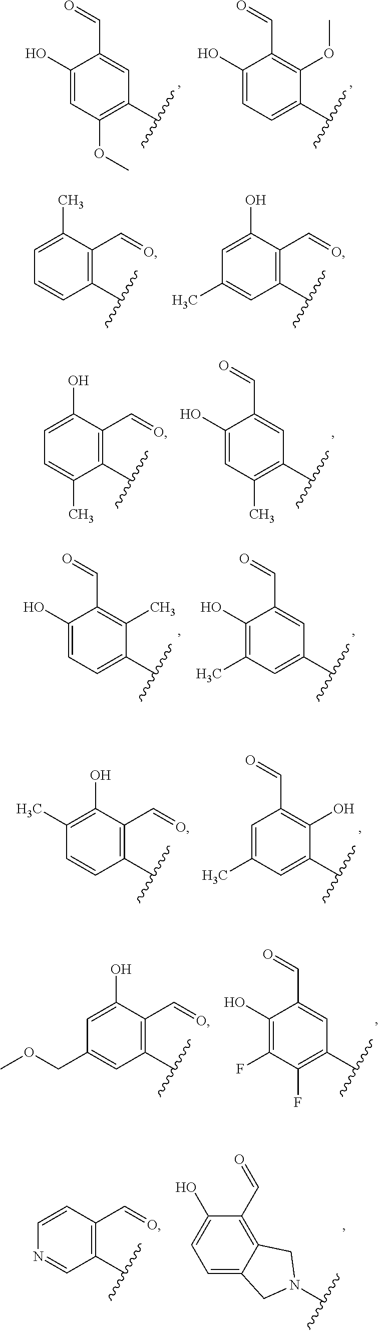

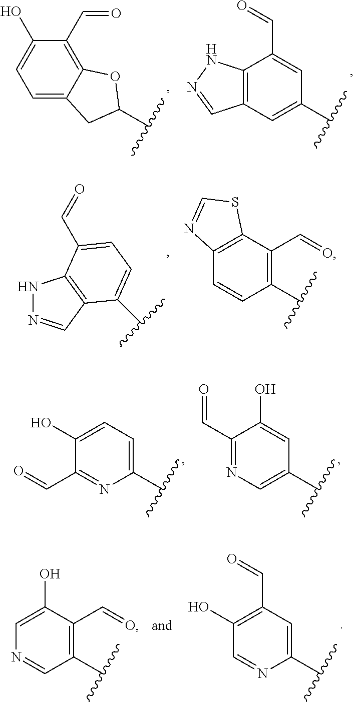

In some embodiments,

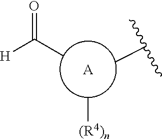

is selected from

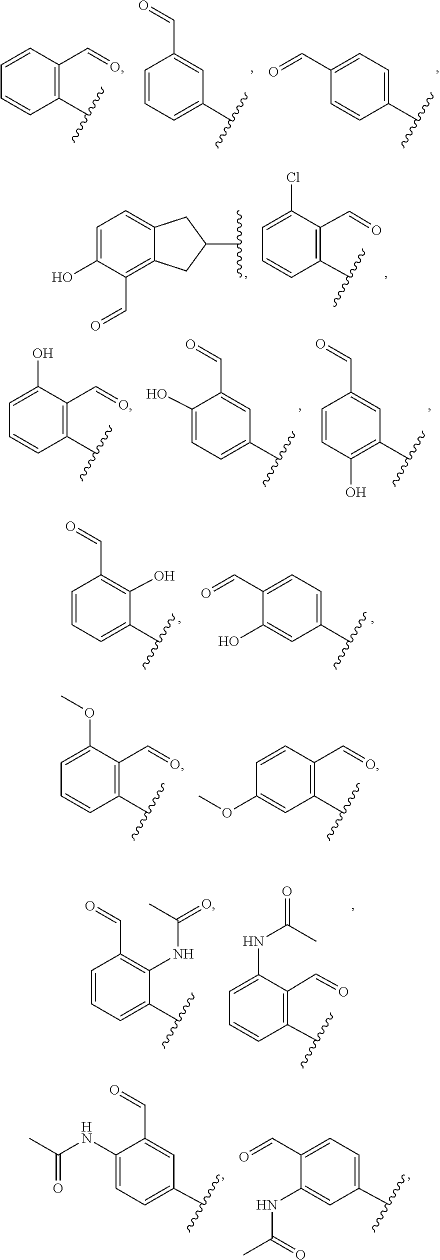

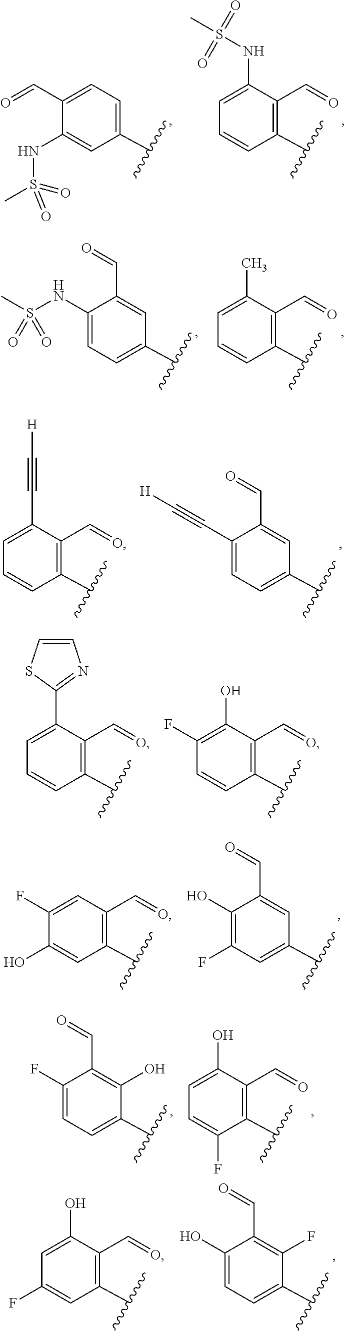

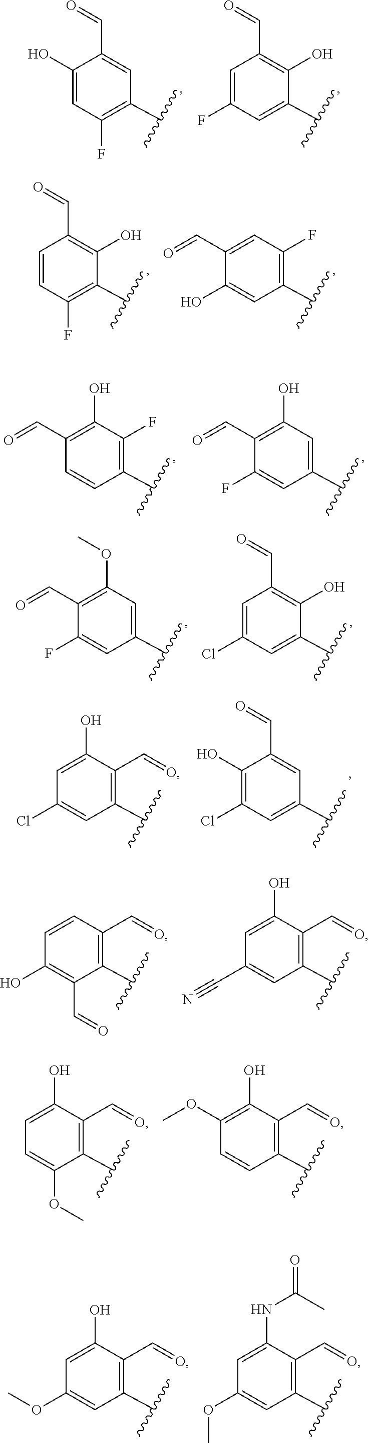

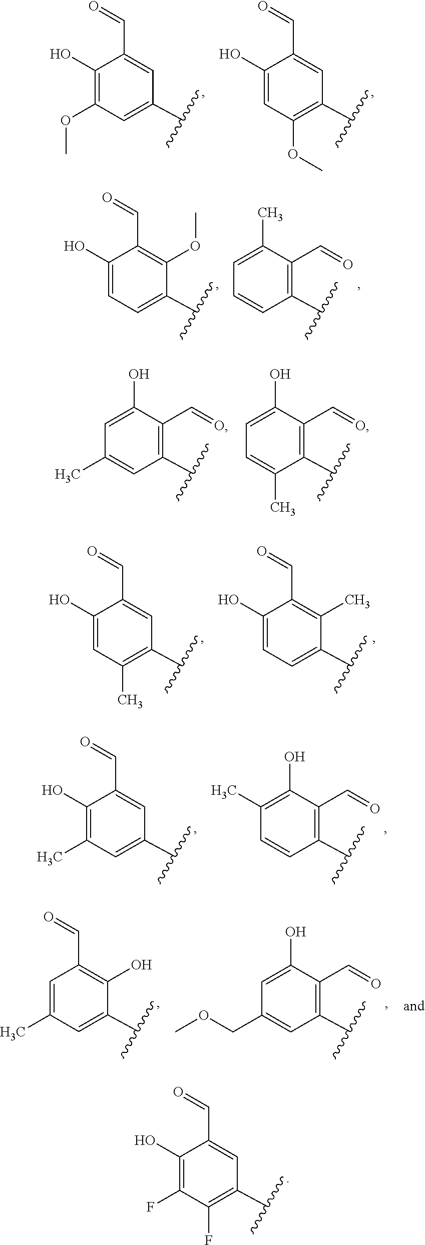

In some embodiments, for the compound or salt of Formula (I), (III), (IV), (IV-A), (IV-B), (IV-C), or (V),

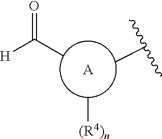

is selected from

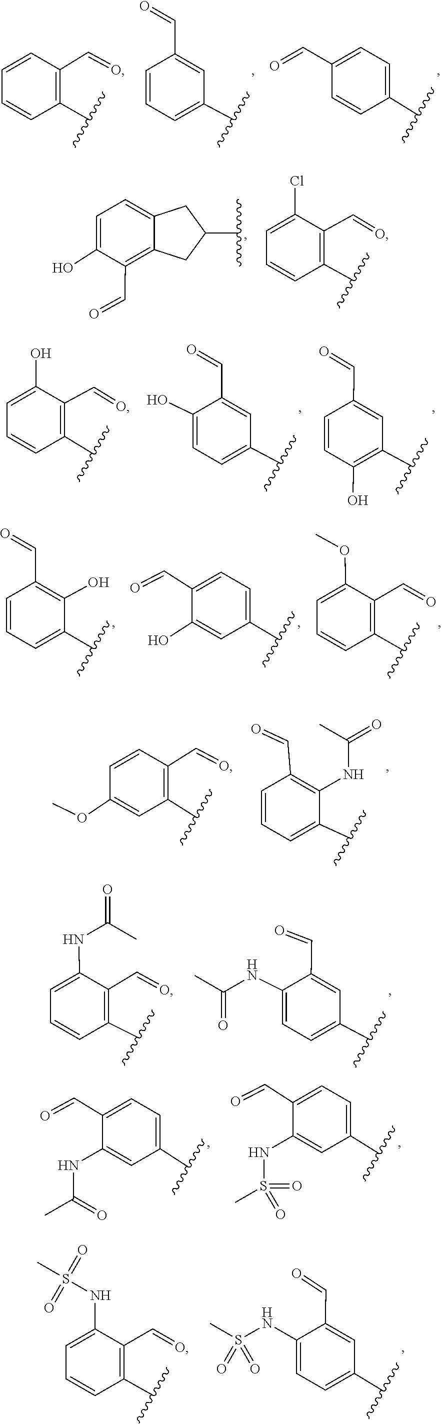

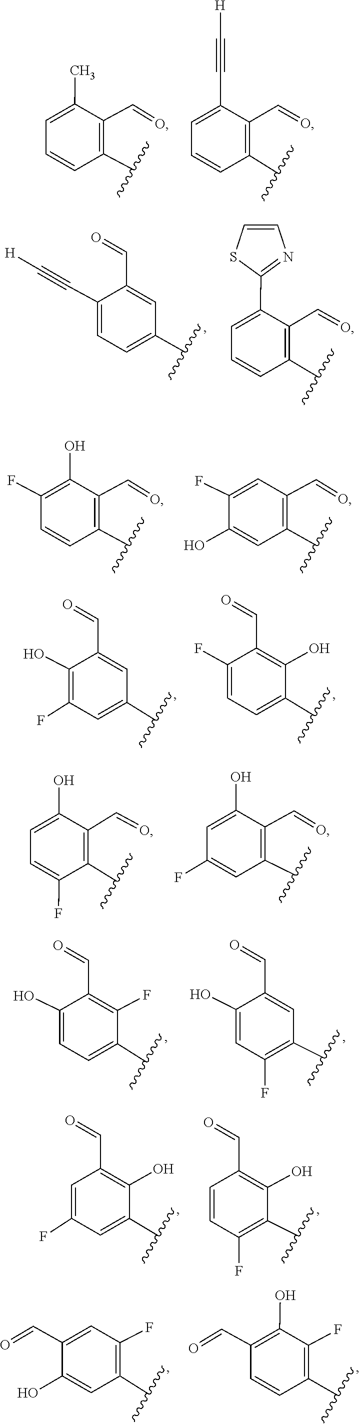

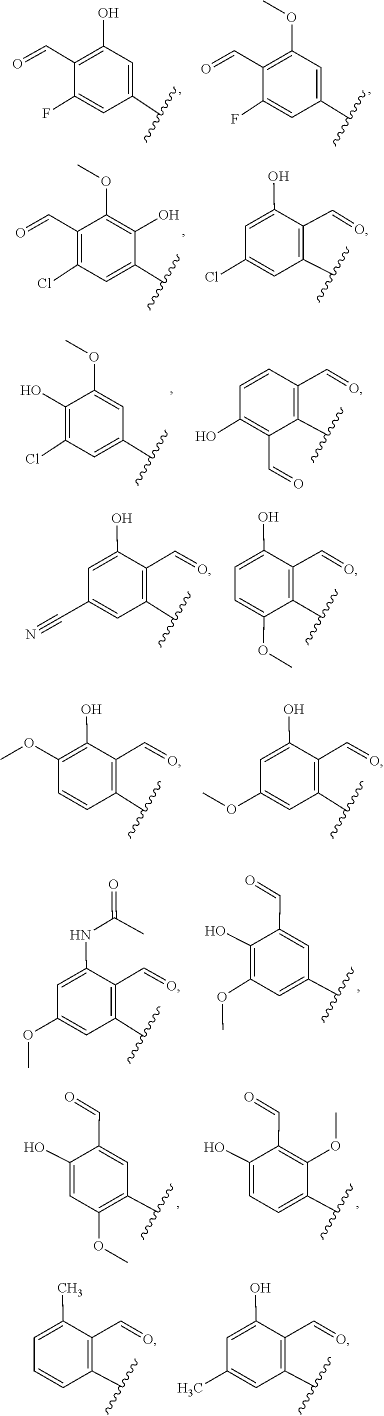

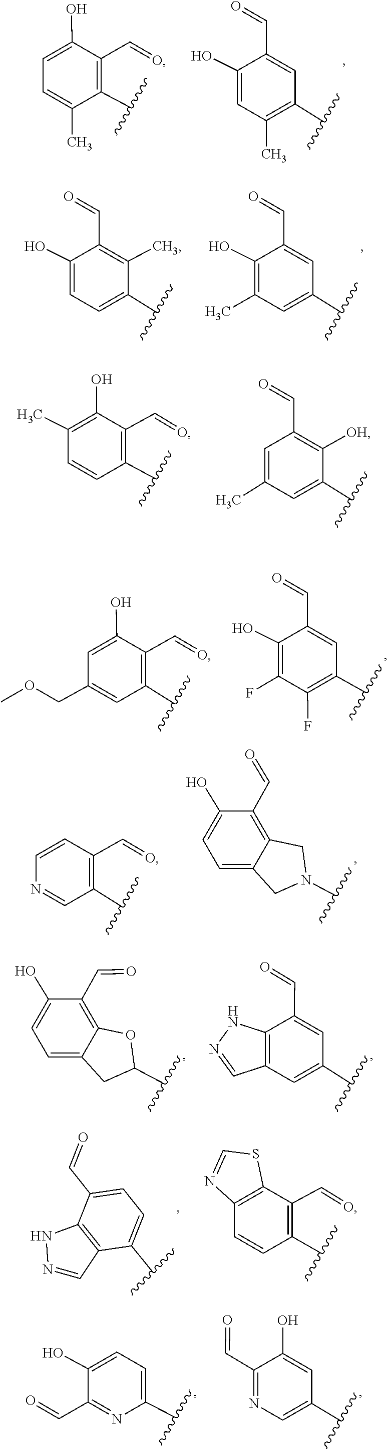

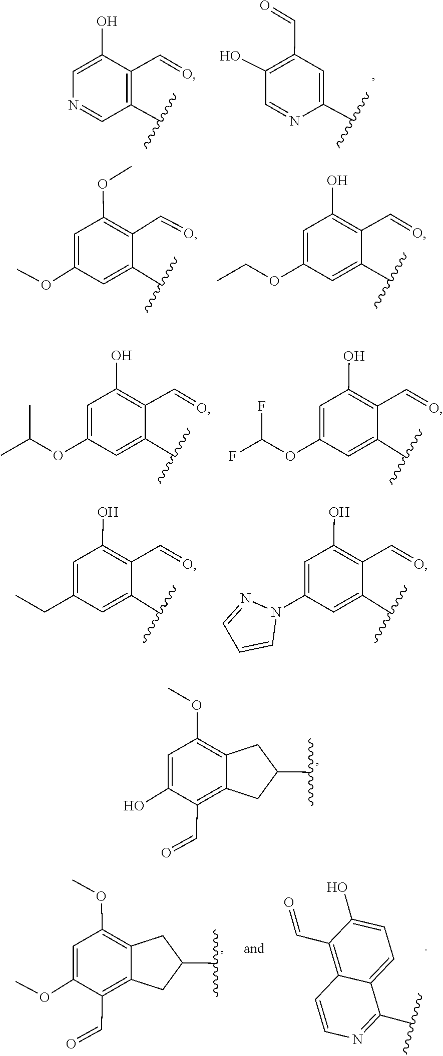

In some embodiments, for the compound or salt of Formula (I), (II), (II-A), (II-B), (II-C), (II-D), (II-E), (II-F), (III), (IV), (IV-A), (IV-B), (IV-C), or (V), any of L¹, L², L³, or L⁴ is independently selected from C₃₋₁₂ carbocyclene. In some embodiments, any of L¹, L², L³, or L⁴ is independently selected from C₃₋₁₂ unsaturated carbocyclene. In some embodiments, any of L¹, L², L³, or L⁴ is independently selected from C₃₋₁₂ arylene. In some embodiments, any of L¹, L², L³, or L⁴ is independently selected from C₃ carbocyclene, C₄ carbocyclene, C₅ carbocyclene, C₆ carbocyclene, C₇ carbocyclene, C₈ carbocyclene, C₉ carbocyclene, C₁₀ carbocyclene, and C₁₁ carbocyclene. In some embodiments, any of L¹, L², L³, or L⁴ is independently selected from C₃₋₄ carbocyclene, C₃₋₅ carbocyclene, C₃₋₆ carbocyclene, C₃₋₇ carbocyclene, C₃₋₈ carbocyclene, C₃₋₉ carbocyclene, C₃₋₁₀ carbocyclene, C₃₋₁₁ carbocyclene, and C₃₋₁₂ carbocyclene. In some embodiments, any of L¹, L², L³, or L⁴ is independently selected C₃₋₈ monocyclic carbocyclene and C₈₋₁₂ bicyclic carbocyclene. In some embodiments, any of L¹, L², L³, or L⁴ is independently selected from C₃₋₈ monocyclic carbocyclene and C₄₋₁₂ bicyclic carbocyclene. In some embodiments, any of L¹, L², L³, or L⁴ is independently selected from C₃₋₈ monocyclic carbocyclene and C₈₋₁₂ bicyclic carbocyclene. In some embodiments, the C₄₋₁₂ carbocyclene is selected from C₄₋₁₂ carbocyclene, C₄₋₁₂ bridged carbocyclene, and C₄₋₁₂ spirocyclic carbocyclene.

In some embodiments, for the compound or salt of Formula (I), (II), (II-A), (II-B), (II-C), (II-D), (II-E), (II-F), (III), (IV), (IV-A), (IV-B), (IV-C), or (V), any of L¹, L², L³, or L⁴ is independently selected from 3- to 12-membered heterocyclene. In some embodiments, any of L¹, L², L³, or L⁴ is independently selected from 3- to 12-membered heterocyclene comprising at least one heteroatom selected from oxygen, nitrogen, and sulfur. In some embodiments, any of L¹, L², L³, or L⁴ is independently selected from 3- to 12-membered unsaturated heterocyclene. In some embodiments, any of L¹, L², L³, or L⁴ is independently selected from 3- to 12-membered heteroarylene. In some embodiments, any of L¹, L², L³, or L⁴ is independently selected from 3-membered heterocyclene, 4-membered heterocyclene, 5-membered heterocyclene, 6-membered heterocyclene, 7-membered heterocyclene, 8-membered heterocyclene, 9-membered heterocyclene, 10-membered heterocyclene, and 11-membered heterocyclene. In some embodiments, any of L¹, L², L³, or L⁴ is independently selected from 3- to 4-membered heterocyclene, 3- to 5-membered heterocyclene, 3- to 6-membered heterocyclene, 3- to 7-membered heterocyclene, 3- to 8-membered heterocyclene, 3- to 9-membered heterocyclene, 3- to 10-membered heterocyclene, 3- to 11-membered heterocyclene, and 3- to 12-membered heterocyclene. In some embodiments, any of L¹, L², L³, or L⁴ is independently selected from 3- to 8-membered monocyclic heterocyclene and 4- to 12-membered bicyclic heterocyclene. In some embodiments, any of L¹, L², L³, or L⁴ is independently selected from 3- to 8-membered monocyclic heterocyclene and 8- to 12-membered bicyclic heterocyclene. In some embodiments, any of L¹, L², L³, or L⁴ is independently selected from 3- to 8-membered monocyclic heterocyclene and 8- to 12-membered bicyclic heterocyclene. In some embodiments, the 4- to 12-membered bicyclic heterocyclene is selected from 4- to 12-membered fused heterocyclene, 4- to 12-membered bridged heterocyclene, and 4- to 12-membered spirocyclic heterocyclene.

In some embodiments, for the compound or salt of Formula (I), (II), (II-A), (II-B), (II-C), (II-D), (II-E), (II-F), (III), (IV), (IV-A), (IV-B), (IV-C), or (V), each of L², L³ and L⁴ are absent and L¹ is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, and C₂₋₆ alkynylene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), each of L², L³ and L⁴ are absent and L¹ is selected from (a) and (b): (a) —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b):(a) —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from (a) and (b): (a) —N(R¹²)C(O)—, —N(R¹²)C(O)C(O)N(R¹²)—, and —(R¹²)NC(O)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from —N(R¹²)C(O)—, —N(R¹²)C(O)C(O)N(R¹²)—, and —(R¹²)NC(O)N(R¹²)—; and R¹² is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), each of L², L³ and L⁴ are absent and L¹ is selected from (a) and (b): (a) —N(R¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L², L³ and L⁴ are absent and L¹ is selected from (a) and (b):(a) —N(R¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from (a) and (b): (a) —N(R¹²)—, —N(R¹²)C(O)—, —N(R¹²)C(O)C(O)N(R¹²)—, and —(R¹²)NC(O)N(R¹²)—; and (b) C₁₋₆ alkylene which is optionally substituted with one or more substituents independently selected from halogen, —OR¹³, —SR¹³, ═O, ═S, —CN. In some embodiments, each of L², L³ and L⁴ are absent and L¹ is selected from —N(R¹²)—, —N(R¹²)C(O)—, —N(R¹²)C(O)C(O)N(R¹²)—, and —(R¹²)NC(O)N(R¹²)—; and R¹² is selected from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), L¹ is selected from —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and R¹² is independently selected at each occurrence from hydrogen, C₁₋₆ alkyl, and C₁₋₆ haloalkyl. In some embodiments, L¹ is selected from —N(R¹²)C(O)—, —N(R¹²)C(O)C(O)N(R¹²)—, and —(R¹²)NC(O)N(R¹²)—; and R¹² is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), L¹ is selected from —N(R¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and R¹² is independently selected at each occurrence from hydrogen, C₁₋₆ alkyl, and C₁₋₆ haloalkyl. In some embodiments, L¹ is selected from —N(R¹²)—, —N(R¹²)C(O)—, —N(R¹²)C(O)C(O)N(R¹²)—, and —(R¹²)NC(O)N(R¹²)—; and R¹² is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), L is selected from:

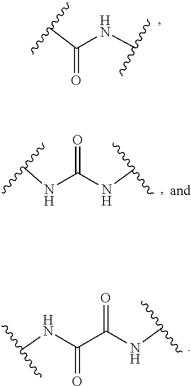 [SMILES: *NC(*)=O ; *NC(=O)N* ; *NC(=O)C(=O)N*]

In some embodiments, L is selected from:

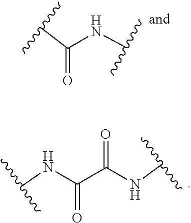 [SMILES: *NC(*)=O ; *NC(=O)C(=O)N*]

In some embodiments, L is

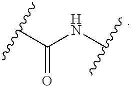 [SMILES: *NC(*)=O]

In some embodiments, L is

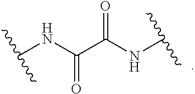 [SMILES: *NC(=O)C(=O)N*]

In some embodiments, L is

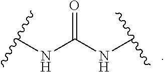 [SMILES: *NC(=O)N*]

In some embodiments, for the compound or salt of Formula (I), (II), (II-A), (II-B), (II-C), (II-D) (II-E), (II-F), (III), (IV), (IV-A), (IV-B), (IV-C), or (V), L is selected from:

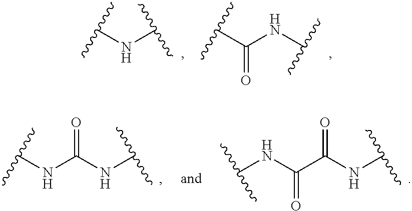

In some embodiments, L is selected from:

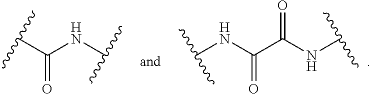 [SMILES: *NC(*)=O ; *NC(=O)C(=O)N*]

In some embodiments, L is

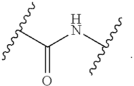 [SMILES: *NC(*)=O]

In some embodiments, L is

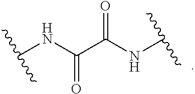 [SMILES: *NC(=O)C(=O)N*]

In some embodiments, L is

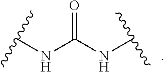 [SMILES: *NC(=O)N*]

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L³ and L⁴ are absent and each of L¹ and L² is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, and C₂₋₆ alkynylene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L³ and L⁴ are absent and each of L¹ and L² is selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —O—, —N(R¹²)—, —N(R¹²)C(O)—, —N(R¹²)C(O)O—, —N(R¹²)S(O)₂—, —N(R¹²)S(O)₂N(R¹²)—, and —(R¹²)NC(O)N(R¹²)—; and (b) C₁₋₆ alkylene, C₂₋₆ alkenylene, C₃₋₁₀ carbocyclene, and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN. In some embodiments, each of L³ and L⁴ are absent and each of L¹ and L² are independently selected from (a) and (b): (a) —O—, —N(R¹²)—, —N(R¹²)C(O)—, and —N(R¹²)C(O)O—; and (b) C₁₋₆ alkylene and C₂₋₆ alkenylene, each of which is optionally substituted with one or more substituents independently selected from halogen, —OR¹³, and —CN. In some embodiments, each of L³ and L⁴ are absent and each of L¹ and L² are independently selected from (a) and (b): (a) —O—, —N(R¹²)—, —N(R¹²)C(O)—, and —N(R¹²)C(O)O—; and (b) C₁₋₆ alkylene and C₂₋₆ alkenylene, each of which is optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, L is selected from:

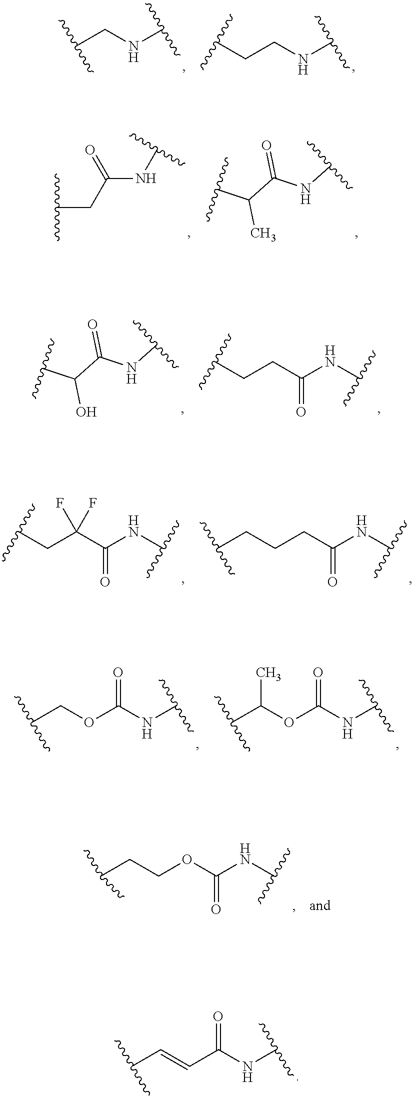

In some embodiments, L is selected from

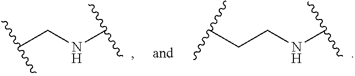

In some embodiments, L is selected from

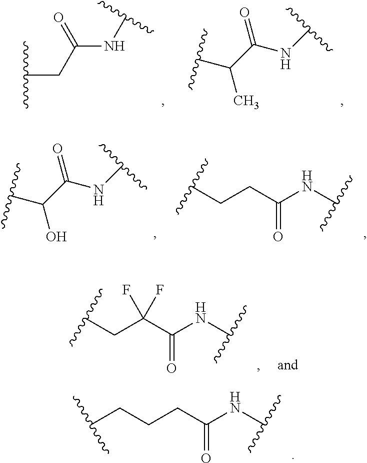

In some embodiments, L is selected from

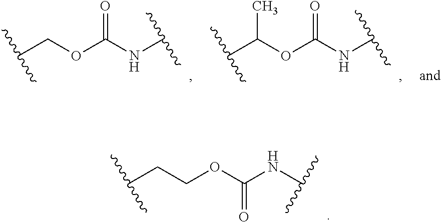

In some embodiments, L is

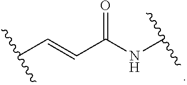 [SMILES: */C=C/C(=O)N*]

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₃₋₁₀ carbocyclene and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) C₃₋₁₀ carbocyclene and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₃₋₁₀ carbocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) C₃₋₁₀ carbocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) C₃₋₆ saturated carbocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, L is selected from

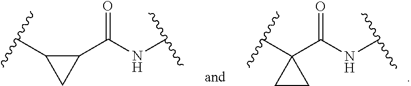 [SMILES: *NC(=O)C1CC1* ; *NC(=O)C1(*)CC1]

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) 3- to 10-membered heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) 3- to 10-membered heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) 3- to 6-membered saturated heterocyclene and 3- to 6-membered unsaturated heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, L is selected from:

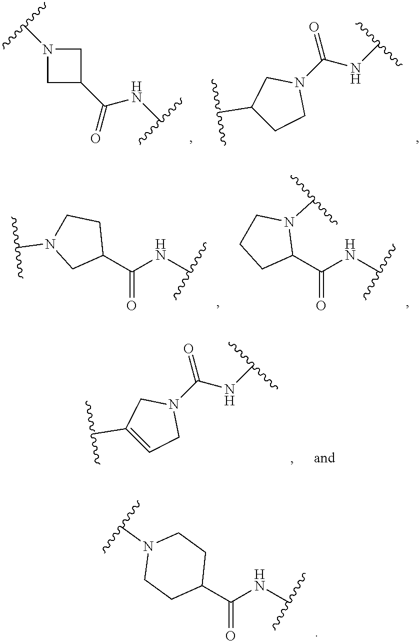 [SMILES: *NC(=O)C1CN(*)C1 ; *NC(=O)N1CCC(*)C1 ; *NC(=O)C1CCN(*)C1 ; *NC(=O)C1CCCN1* ; *NC(=O)N1CC=C(*)C1 ; *NC(=O)C1CCN(*)CC1]

In some embodiments, for the compound or salt of Formula (I), (II), (II-A), (II-B), (II-C), (II-D), (II-E), (II-F), (III), (IV), (IV-A), (IV-B), (IV-C), or (V), each of L³ and L⁴ are absent and each of L¹ and L² are independently selected from (a) and (b): (a) —N(R¹²)—, —N(R¹²)C(O)—, —N(R¹²)C(O)O—, and —(R¹²)NC(O)N(R¹²)—; and (b) 5- to 6-membered heteroarylene and 6- to 10-membered bicyclic heterocyclene optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, and —CN; and R¹³ is independently selected at each occurrence from hydrogen, C₁₋₆ alkyl, and C₁₋₆ haloalkyl. In some embodiments, L is selected from

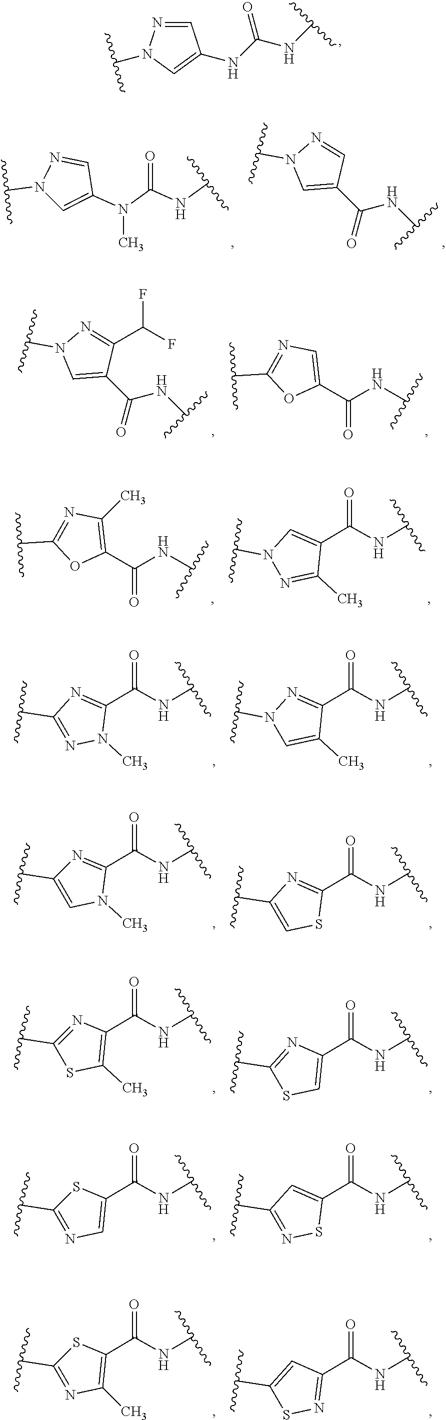

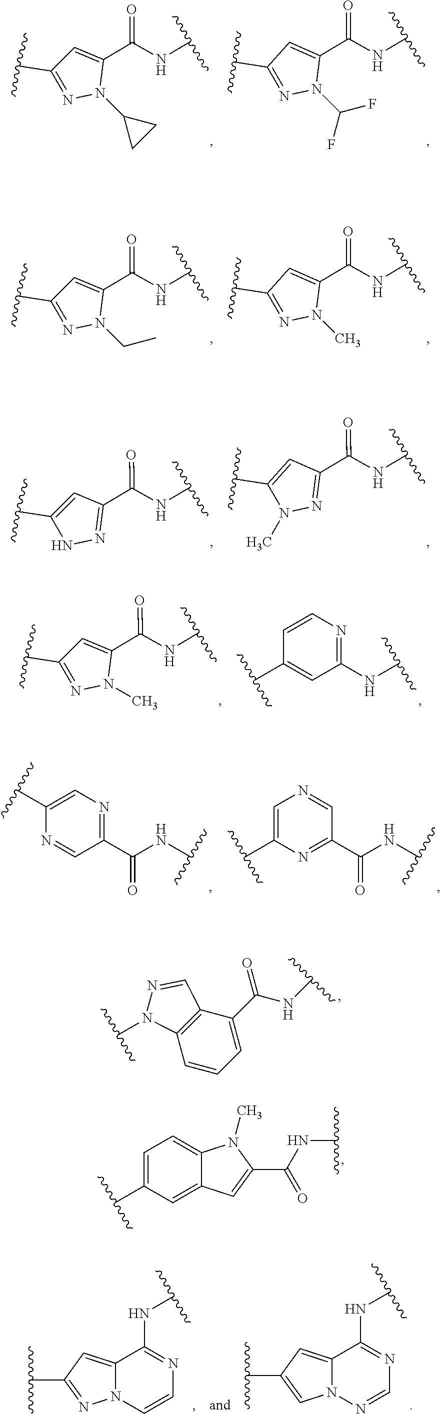

In some embodiments, L is selected from

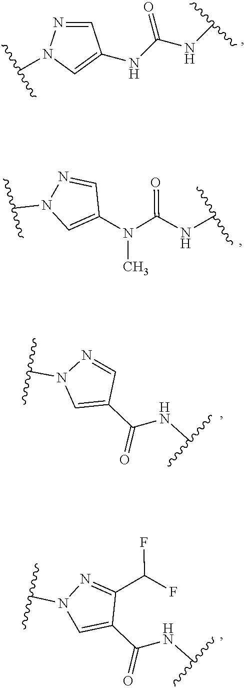 [SMILES: *NC(=O)Nc1cnn(*)c1 ; *NC(=O)N(C)c1cnn(*)c1 ; *NC(=O)c1cnn(*)c1 ; *NC(=O)c1cn(*)nc1C(F)F]

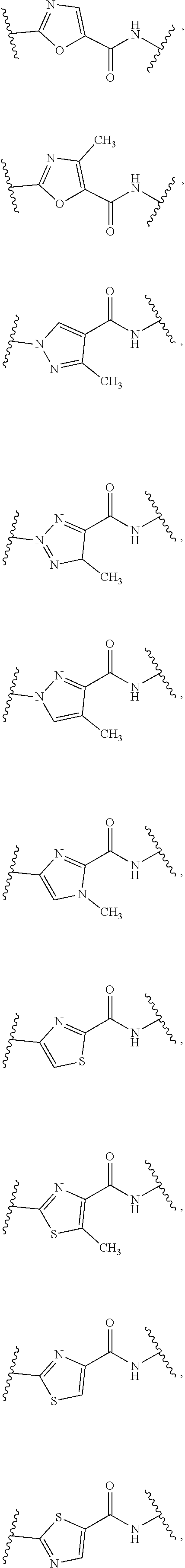

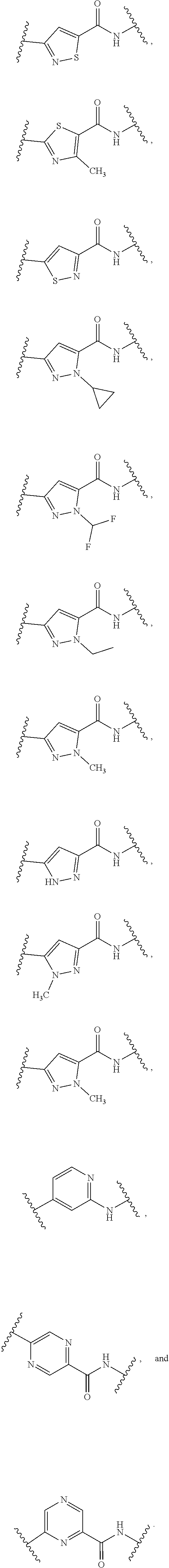

In some embodiments, L is selected from

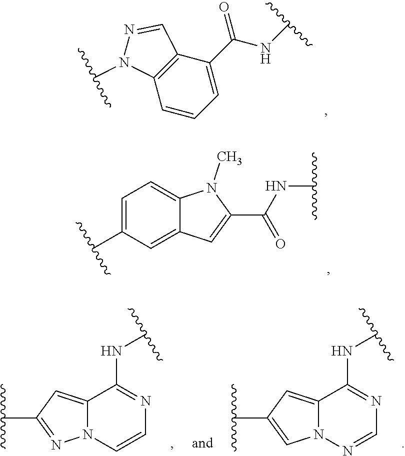

In some embodiments, for the compound or salt of Formula (I), (II), (II-A), (II-B), (II-C), (II-D), (II-E), (II-F), (III), (IV), (IV-A), (IV-B), (IV-C), or (V), L is selected from:

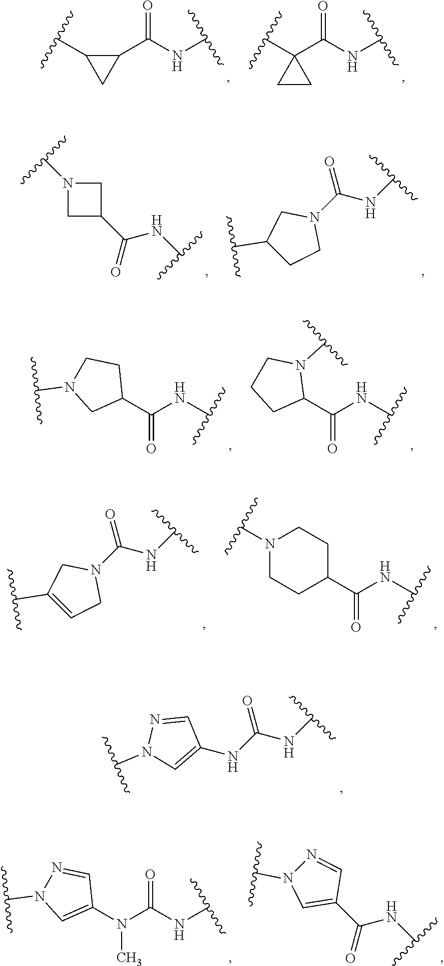

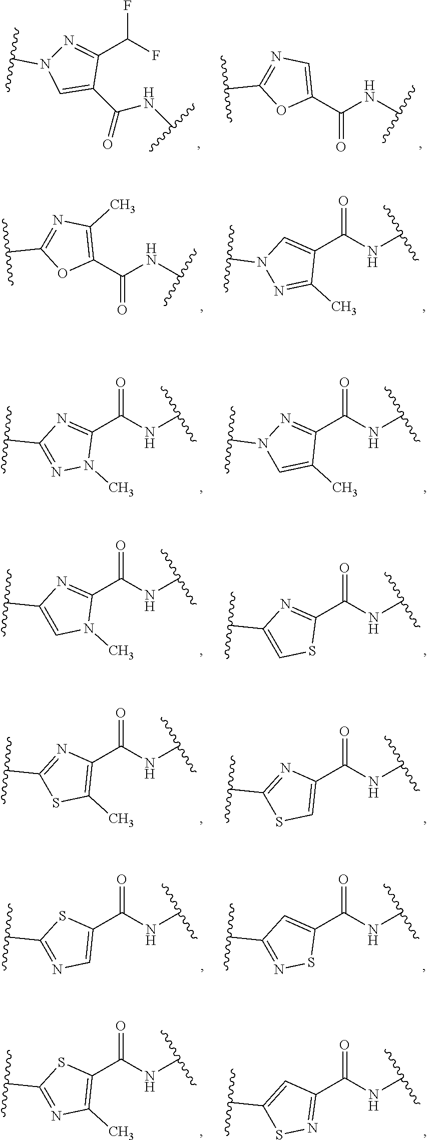

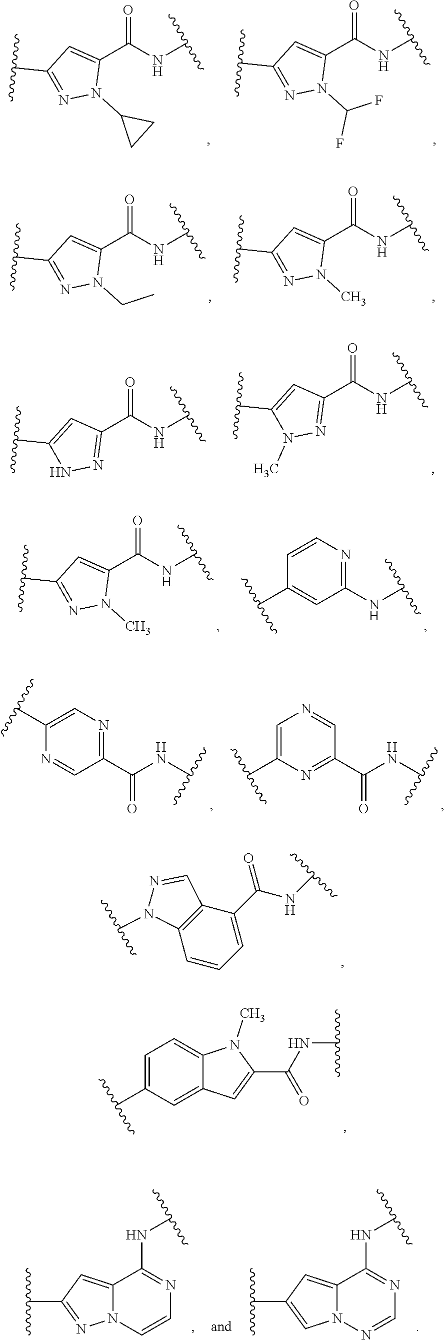

In some embodiments, for the compound or salt of Formula (I), (II), (II-A), (II-B), (II-C), (II-E), (II-E), (III), (IV), (IV-A), (IV-B), (IV-C), or (V), L is selected from:

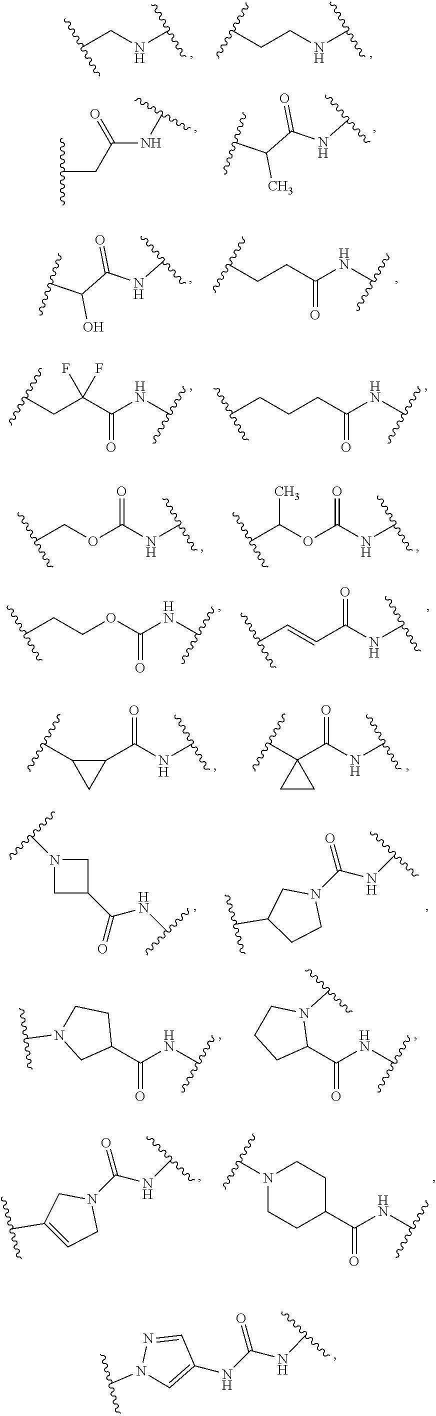

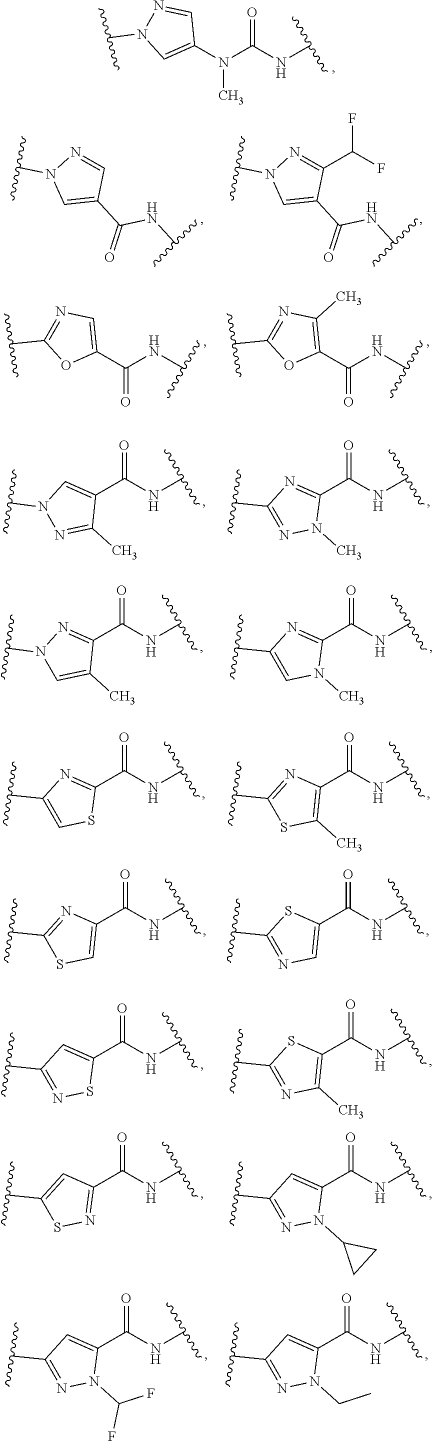

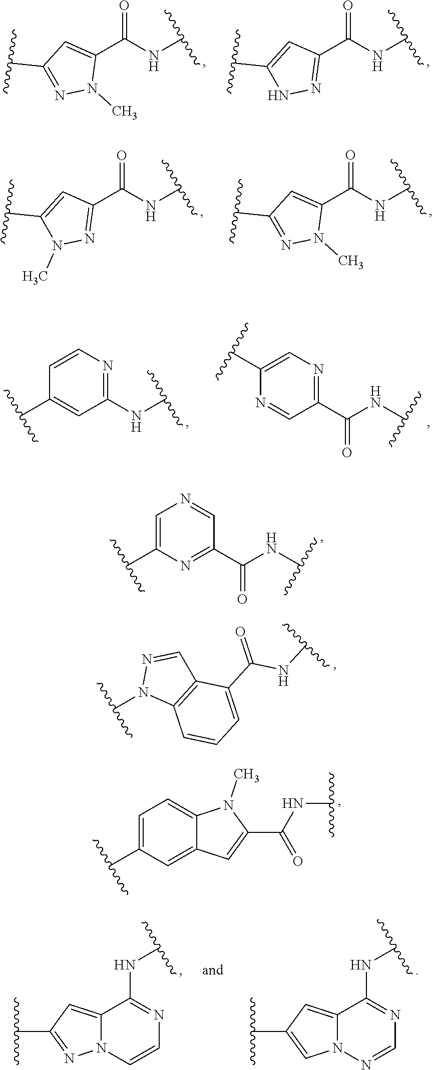

In some embodiments, for the compound or salt of Formula (I), (II), (II-A), (II-B), (II-C), (II-D), (II-E), (II-F), (III), (IV), (IV-A), (IV-B), (IV-C), or (V), L is selected from:

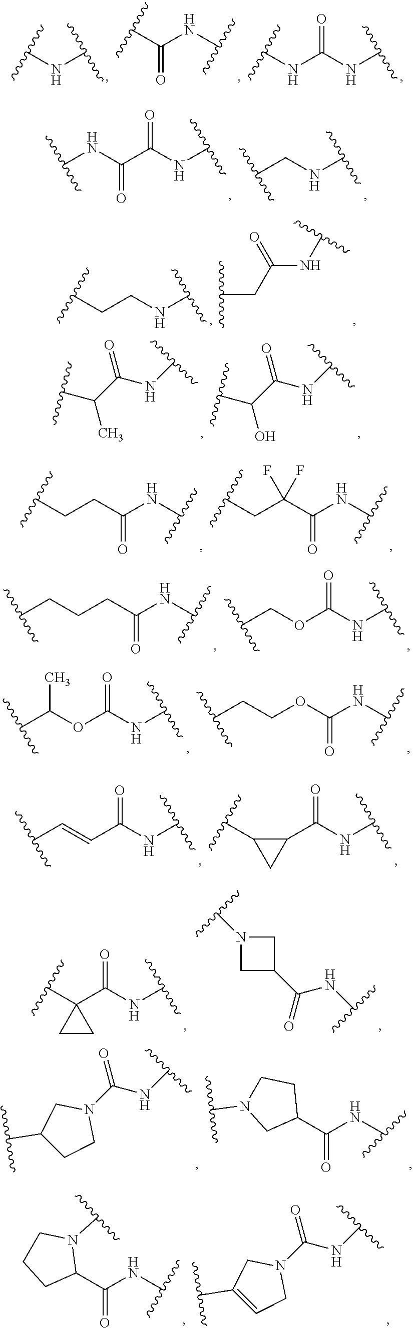

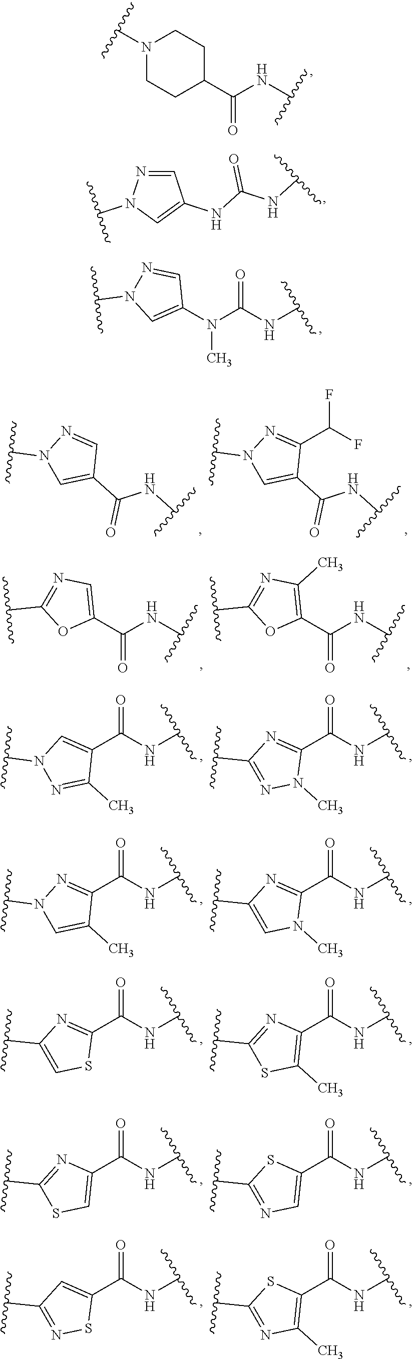

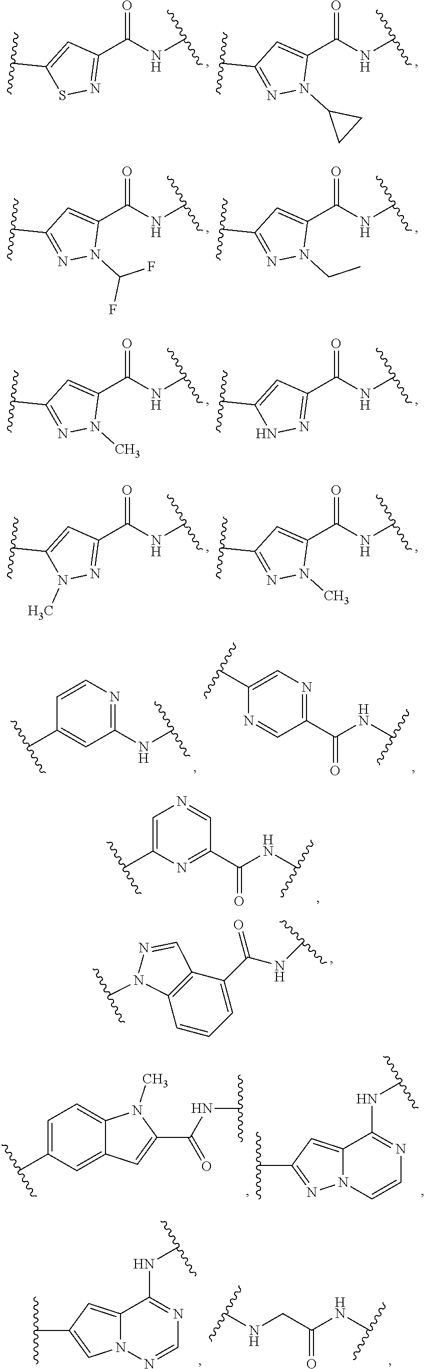

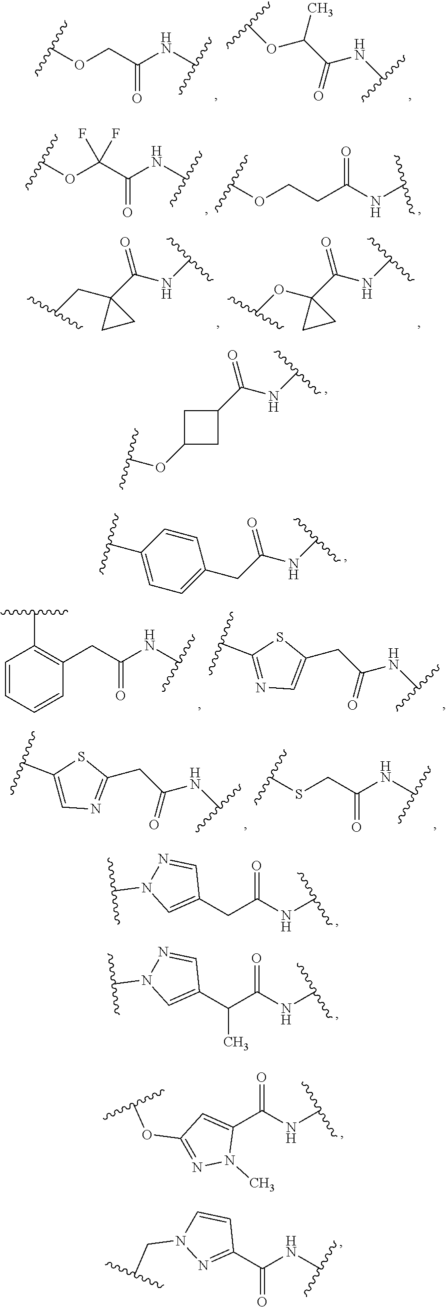

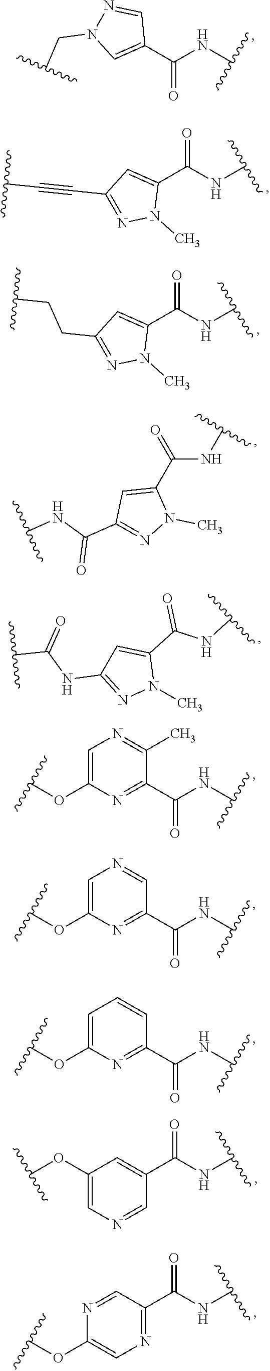

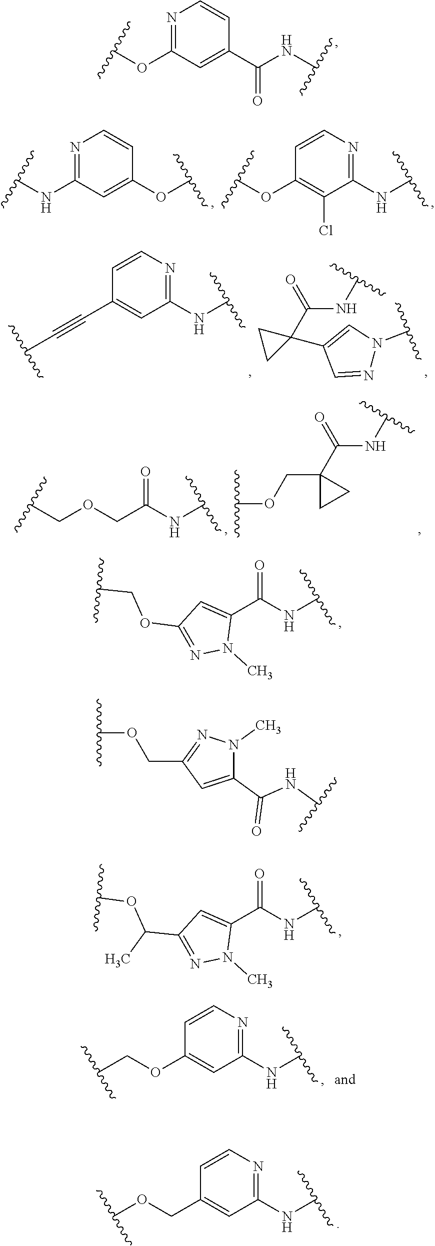

In some embodiments, for the compound or salt of Formula (I), (II), (II-A), (II-B), (II-C), (II-D), (II-E), (II-F), (III), (IV), (IV-A), (IV-B), (IV-C), or (V), L⁴ is absent and each of L¹, L², and L³ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, L⁴ is absent and each of L¹, L², and L³ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, and C₂₋₆ alkynylene, any one of which is optionally substituted with one or more substituents independently selected from halogen, —OR¹³, —SR¹³ ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, L⁴ is absent and each of L¹, L², and L³ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), L⁴ is absent and each of L¹, L², and L³ are independently selected from (a) and (b): (a) —O—, —N(R¹²)—, —S—, —N(R¹²)C(O)—, and —N(R¹²)C(O)O—; and (b) C₁₋₆ alkylene, C₂₋₆ alkynylene, C₃₋₁₀ carbocyclene, and 3- to 10-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, halogen, —OR¹³, —CN, C₁₋₆ alkyl; and R¹³ is independently selected at each occurrence from hydrogen, C₁₋₆ alkyl, and C₁₋₆ haloalkyl. In some embodiments, L⁴ is absent and each of L¹, L², and L³ are independently selected from (a) and (b): (a) —O—, —N(R¹²)—, —S—, —N(R¹²)C(O)—, and —N(R¹²)C(O)O—; and (b) C₁₋₆ alkylene, C₂₋₆ alkynylene, C₃₋₆ carbocyclene, and 3- to 6-membered heterocyclene, any one of which is optionally substituted with one or more substituents independently selected from halogen, —OR¹³, —CN, C₁₋₆ alkyl and C₃₋₆ carbocycle, wherein the C₁₋₆ alkyl and C₃₋₆ carbocycle are each optionally substituted with one or more substituents independently selected from halogen, halogen, —OR¹³, —CN, C₁₋₆ alkyl; and R¹³ is independently selected at each occurrence from hydrogen, C₁₋₆ alkyl, and C₁₋₆ haloalkyl.

In some embodiments, for the compound or salt of Formula (I), (II), (II-A), (II-B), (II-C), (II-D) (II-E), (II-F), (III), (IV), (IV-A), (IV-B), (IV-C), or (V), L is selected from:

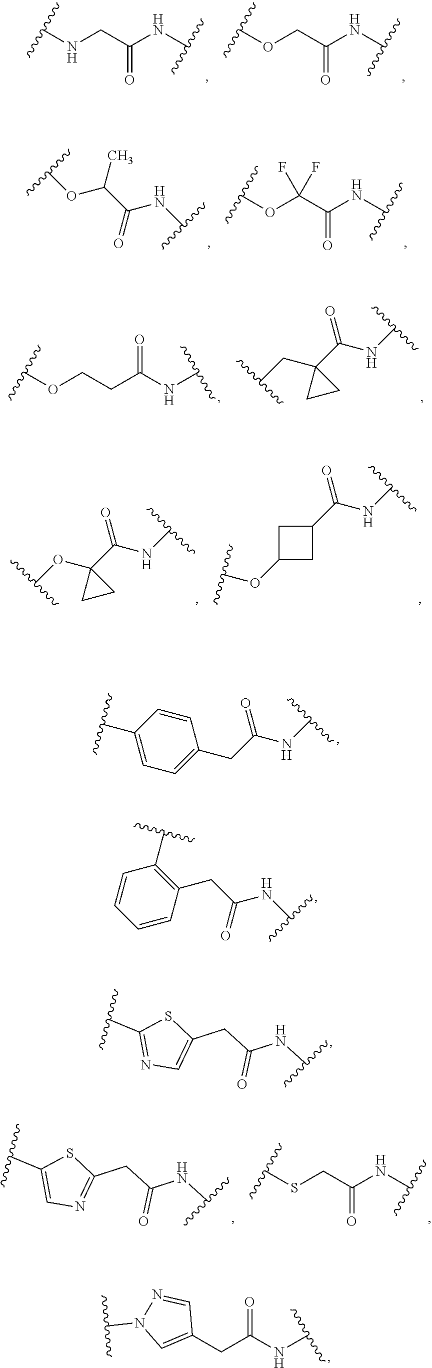

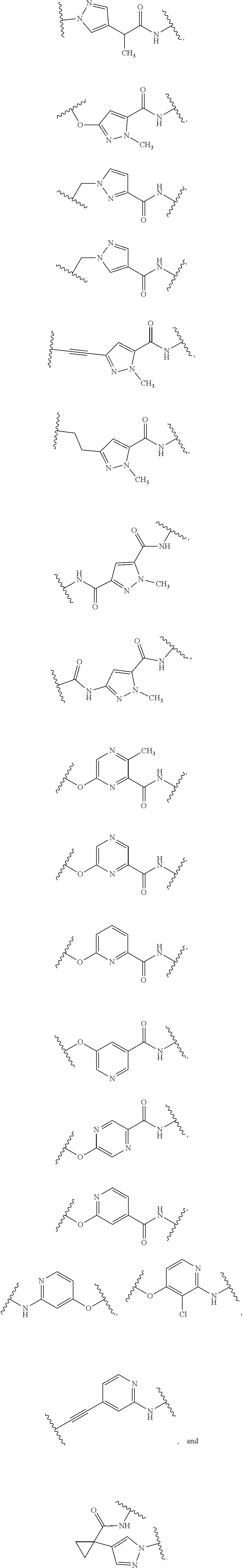

In some embodiments, L is selected from

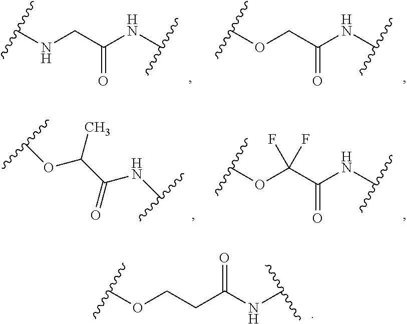

In some embodiments, L is selected from

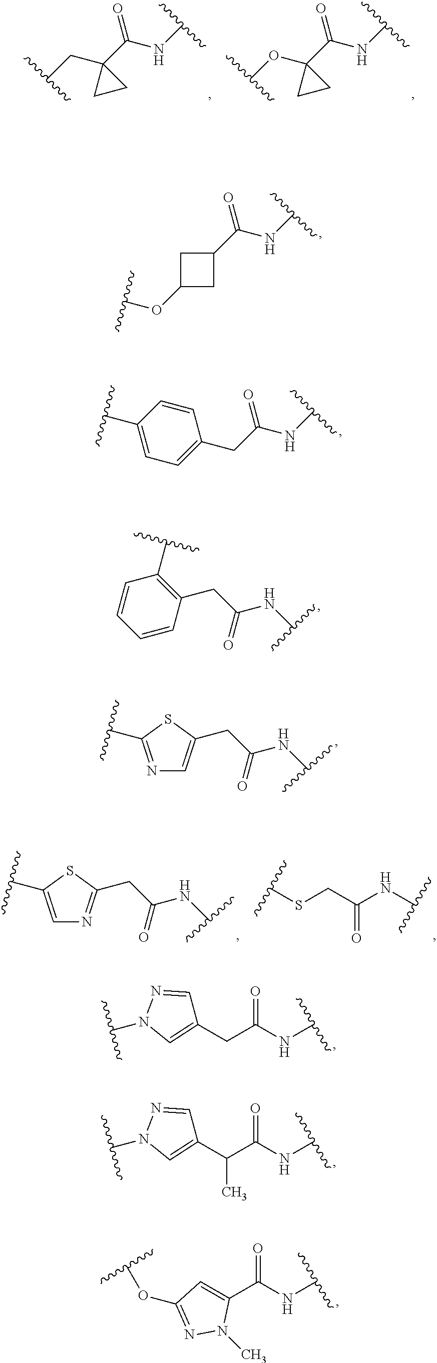

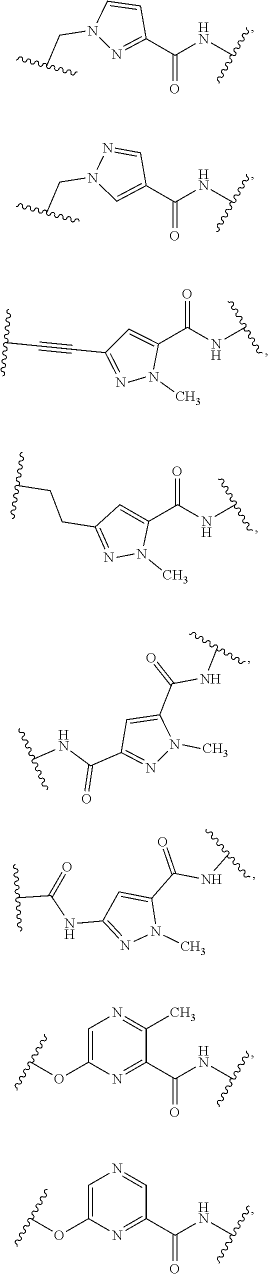

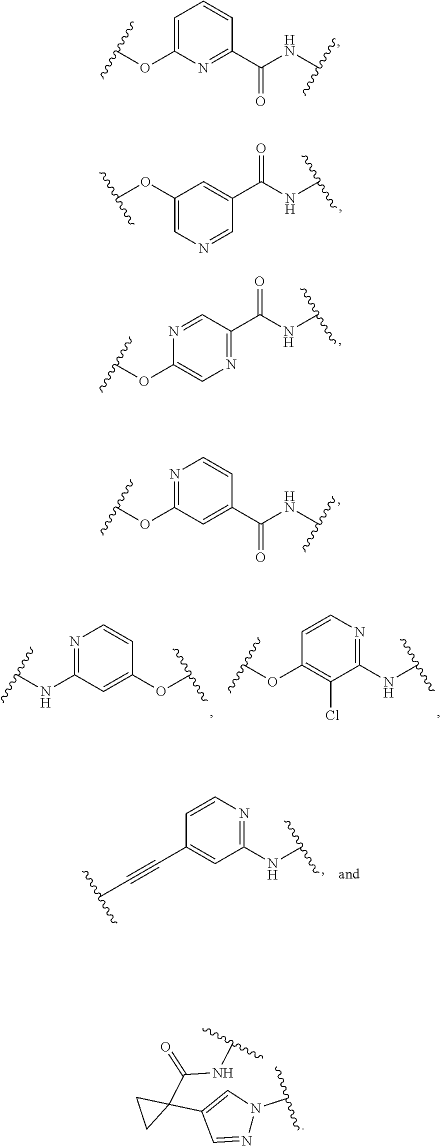

In some embodiments, for the compound or salt of Formula (I), (II), (II-A), (II-B), (II-C), (II-D), (II-E), (II-F), (III), (IV), (IV-A), (IV-B), (IV-C), or (V), each of L¹, L², L³ and L⁴ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some embodiments, for the compound or salt of Formula (I), (II), (II-A), (II-B), (II-C), (II-D), (II-E), (II-F), (III), (IV), (IV-A), (IV-B), (IV-C), or (V), each of L¹, L², L³ and L⁴ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN; and R¹³ is independently selected at each occurrence from hydrogen, C₁₋₆ alkyl, and C₁₋₆ haloalkyl. In some embodiments, each of L¹, L², L³ and L⁴ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₂₋₆ alkenylene, and C₂₋₆ alkynylene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, each of L¹, L², L³ and L⁴ are independently selected from (a) and (b): (a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and (b) C₁₋₆ alkylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN. In some embodiments, L is selected from:

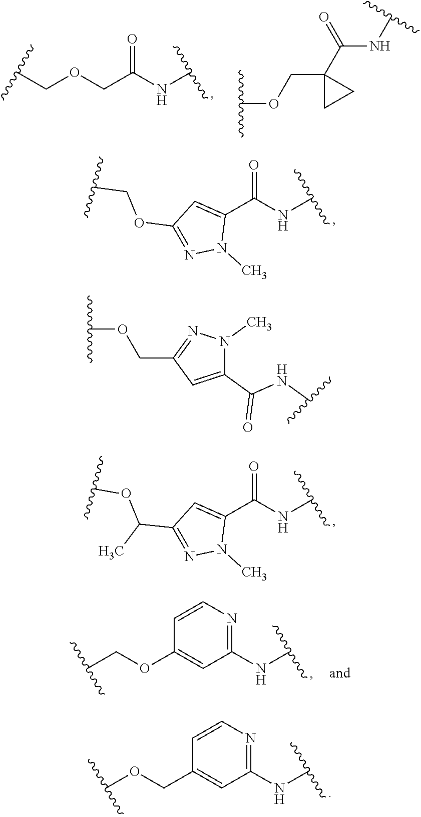

In some embodiments, for the compound or salt of Formula (I), (II), (II-A), (II-B), (II-C), (II-D), (II-E), (II-F), (III), (IV), (IV-A), (IV-B), (IV-C), or (V), L is selected from:

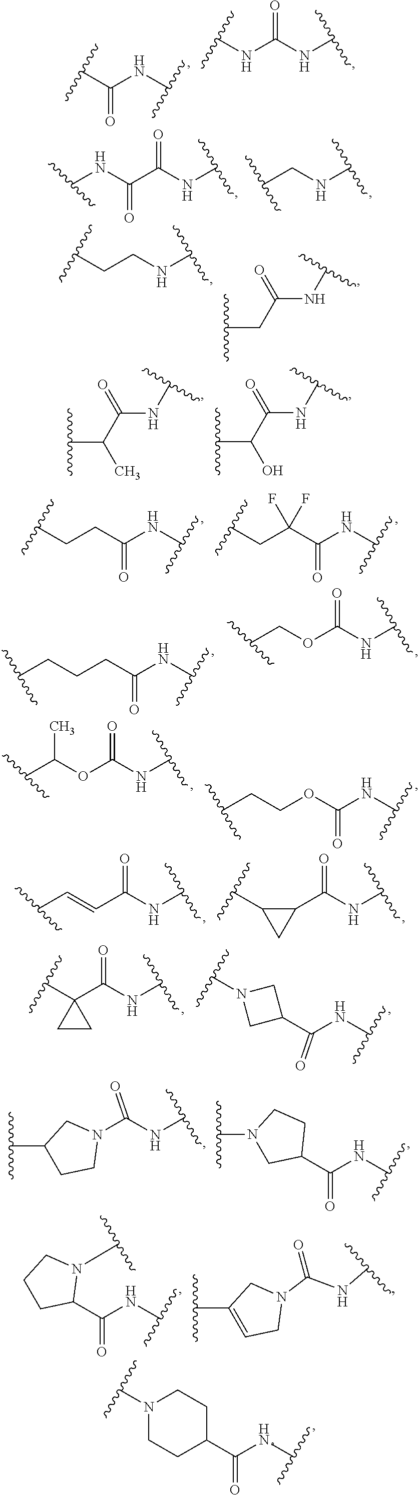

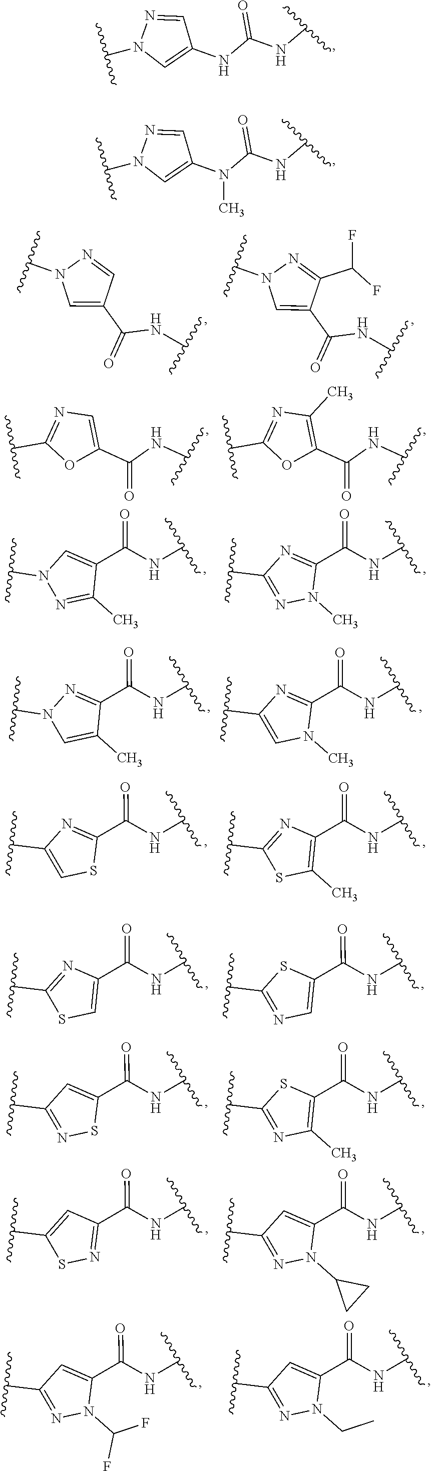

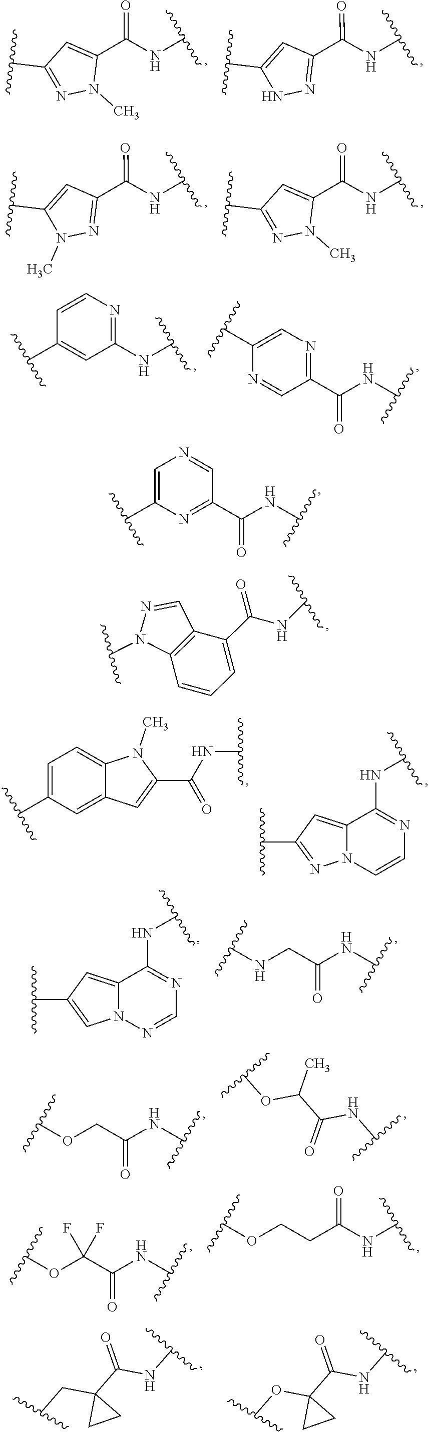

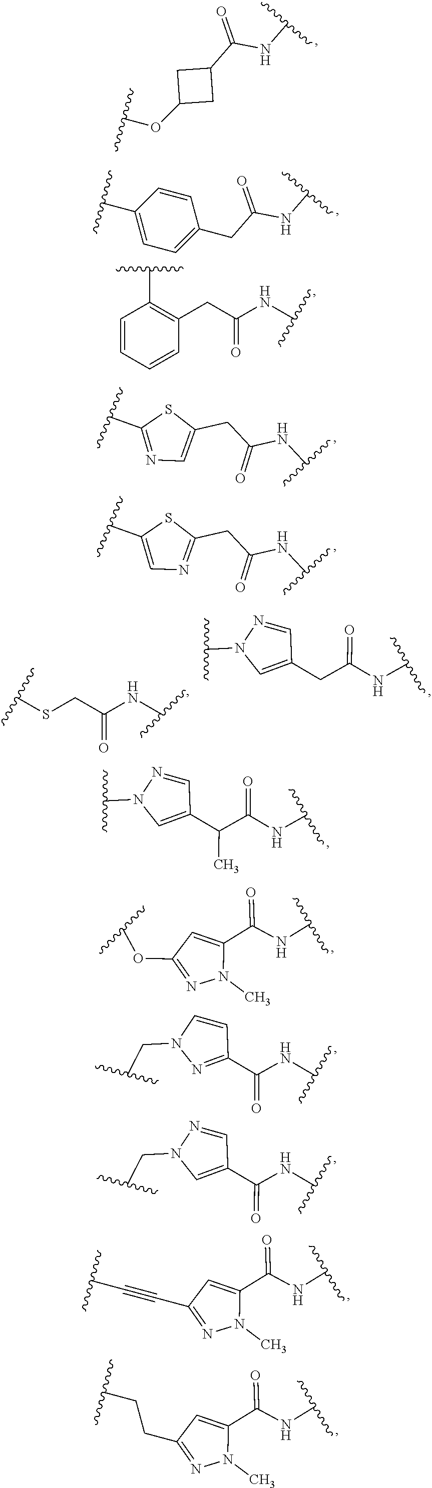

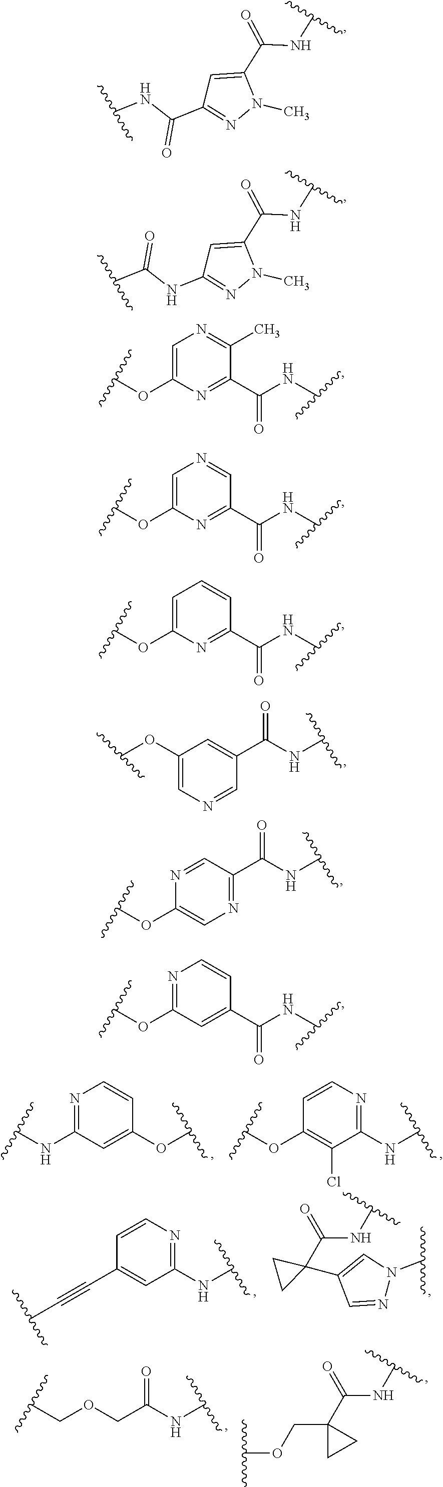

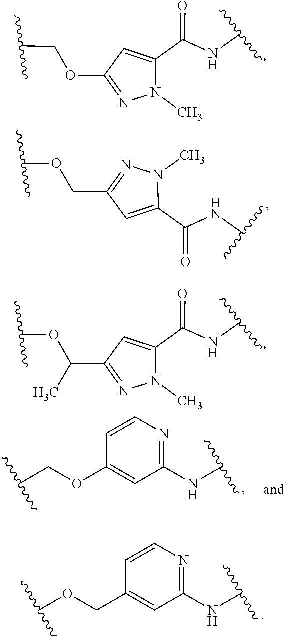

In some embodiments, for the compound or salt of Formula (I), (II), (II-A), (II-B), (II-C), (II-D), (II-E), (II-F), (III), (IV), (IV-A), (IV-B), (IV-C), or (V), one of R¹ or R² is hydrogen.

In some embodiments, for the compound or salt of Formula (I), (II), (II-A), (II-B), (II-C), (II-D), (II-E), (II-F), (III), (IV), (IV-A), (IV-B), (IV-C), or (V), one of R¹ or R² is selected from hydrogen and the other of R¹ or R² is selected from C₁₋₆ alkyl optionally substituted with one or more substituents independently selected from: halogen, —OR¹, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and C₃₋₆ carbocycle and 3- to 6-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ or R² is selected from hydrogen and the other of R¹ or R² is selected from C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and C₃₋₆ carbocycle optionally substituted with one or more substituents independently selected from: halogen, —OR¹⁴, —N(R¹⁴)₂, —C(O)R¹⁴, —NO₂, —CN; and C₁₋₆ alkyl optionally substituted with one or more substituents independently selected from: halogen, —OR¹⁴, —N(R¹⁴)₂, —C(O)R¹⁴, —NO₂, —CN; and R¹⁴ is independently selected at each occurrence from hydrogen, C₁₋₆ alkyl, and C₁₋₆ haloalkyl. In some embodiments, one of R¹ or R² is selected from hydrogen and the other of R¹ or R² is selected from C₁₋₆ alkyl optionally substituted with one or more substituents independently selected from: halogen, —OR¹⁴, —N(R¹⁴)₂, —C(O)R¹⁴, —NO₂, and —CN; and C₃₋₆ carbocycle optionally substituted with one or more substituents independently selected from: halogen, —OR¹⁴, —N(R¹⁴)₂, —C(O)R¹⁴, —NO₂, and —CN; and C₁₋₆ alkyl optionally substituted with one or more substituents independently selected from halogen, —OR¹⁴, —N(R¹⁴)₂, —C(O)R¹⁴, —NO₂, and —CN; and R¹⁴ is independently selected at each occurrence from hydrogen, C₁₋₆ alkyl, and C₁₋₆ haloalkyl. In some embodiments, one of R¹ or R² is selected from hydrogen and the other of R¹ or R² is selected from

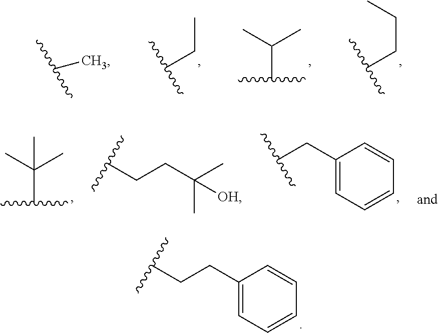

In some embodiments, one of R¹ or R² is selected from hydrogen and the other of R¹ or R² is selected from

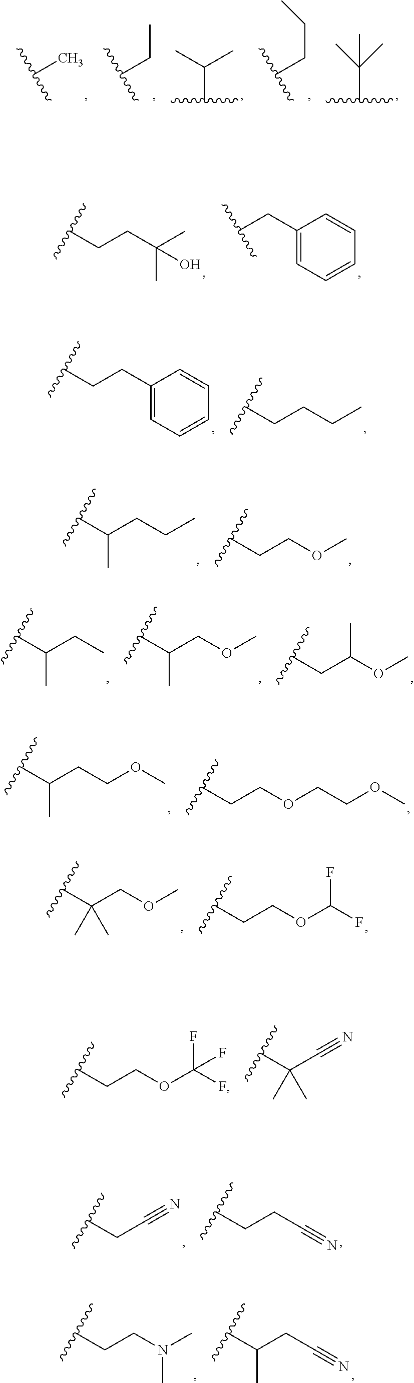

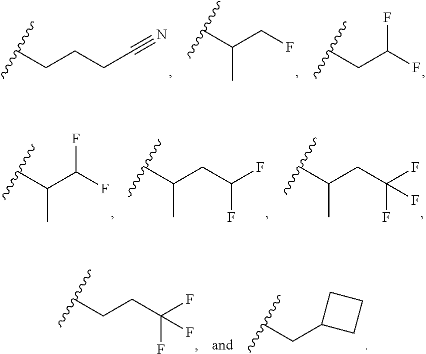

some embodiments, one of R¹ or R² is selected from hydrogen and the other of R¹ or R² is selected from

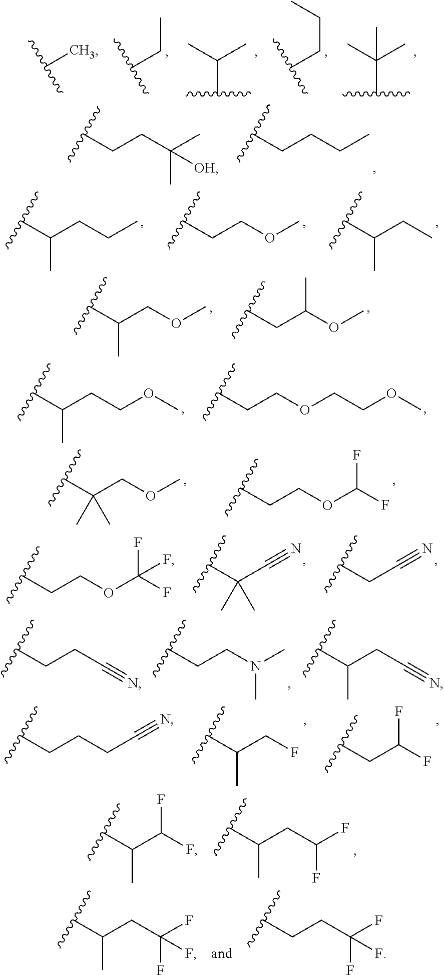

In some embodiments, for the compound or salt of Formula (I), (II), (II-A), (II-B), (II-C), (II-D), (II-E), (II-F), (III), (IV), (IV-A), (IV-B), (IV-C), or (V), one of R¹ or R² is selected from hydrogen and the other of R¹ or R² is selected from C₁₋₆ alkyl optionally substituted with one or more substituents independently selected from: halogen, —OR⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and C₃₋₆ carbocycle and 3- to 6-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ or R² is selected from hydrogen and the other of R¹ or R² is selected from C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, S, ═N(R¹⁴), and —CN; and 3- to 6-membered heterocycle optionally substituted with one or more substituents independently selected from: halogen, —OR¹⁴, —N(R¹⁴)₂, —C(O)R¹⁴, —NO₂, —CN; and C₁₋₆ alkyl optionally substituted with one or more substituents independently selected from: halogen, —OR¹⁴, —N(R¹⁴)₂, —C(O)R¹⁴, —NO₂, —CN. In some embodiments, one of R¹ or R² is selected from hydrogen and the other of R¹ or R² is selected from C₁₋₆ alkyl optionally substituted with one or more substituents independently selected from: halogen, —OR¹⁴, —N(R¹⁴)₂, —C(O)R¹⁴, —NO₂, and —CN; and 3- to 6-membered heterocycle optionally substituted with one or more substituents independently selected from: halogen, —OR¹⁴, —N(R¹⁴)₂, —C(O)R¹⁴, —NO₂, and —CN; and C₁₋₆ alkyl optionally substituted with one or more substituents independently selected from halogen, —OR¹⁴, —N(R¹⁴)₂, —C(O)R¹⁴, —NO₂, and —CN; and R¹⁴ is independently selected at each occurrence from hydrogen, C₁₋₆ alkyl, and C₁₋₆ haloalkyl. In some embodiments, one of R¹ or R² is selected from hydrogen and the other of R¹ or R² is selected from

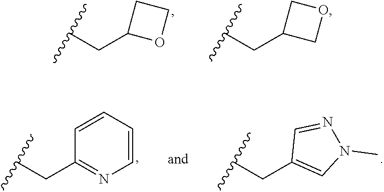

In some embodiments, for the compound or salt of Formula (I), (II), (II-A), (III), (II-a), one of R¹ or R² is selected from hydrogen and the other of R¹ or R² is selected from

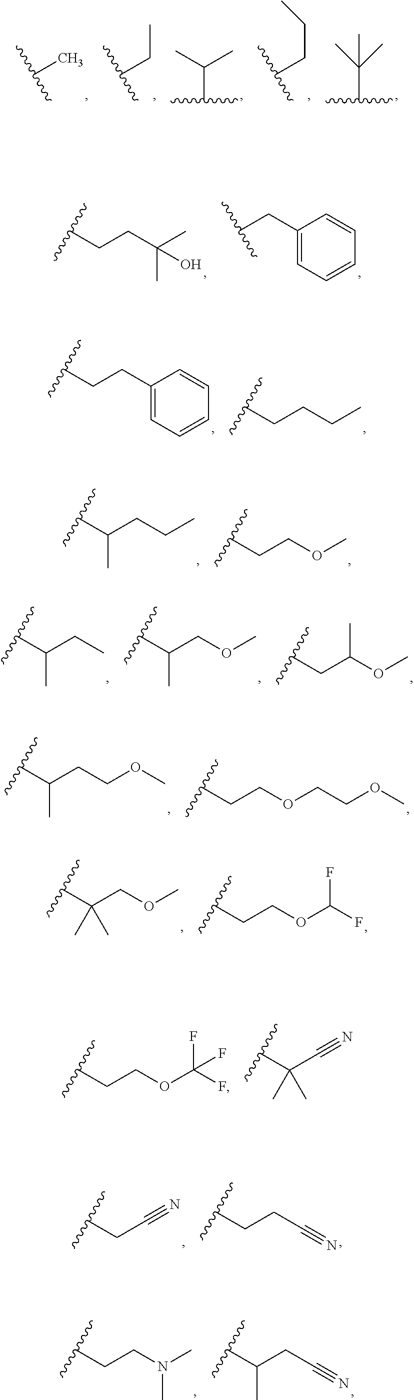

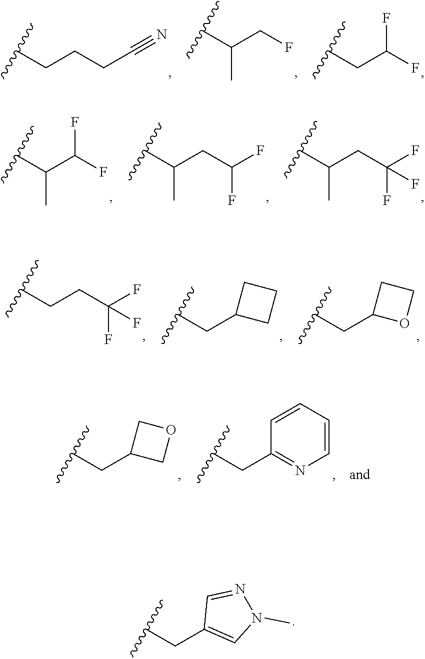

In some embodiments, for the compound or salt of Formula (I), (II), (II-A), (II-B), (II-C), (II-D), (II-E), (II-F), (III), (IV), (IV-A), (IV-B), (IV-C), or (V), one of R¹ or R² is selected from hydrogen and the other of R¹ or R² is selected from C₃₋₆ carbocycle and 3- to 6-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN. In some embodiments, one of R¹ or R² is selected from hydrogen and the other of R¹ or R² is selected from C₃₋₆ carbocycle and 3- to 6-membered heterocycle, each of which is optionally substituted with one or more substituents independently selected from: halogen, —OR¹⁴, —N(R¹⁴)₂, —C(O)R¹⁴, —NO₂, ═O, ═S, ═N(R¹⁴), and —CN; and C₁₋₆ alkyl optionally substituted with one or more substituents independently selected from halogen, —OR¹⁴, —N(R¹⁴)₂, —C(O)R¹⁴, —NO₂, ═O, ═S, ═N(R¹⁴), and —CN. In some embodiments, one of R¹ or R² is selected from hydrogen and the other of R¹ or R² is selected from C₃₋₆ carbocycle and 3- to 6-membered heterocycle, each of which is optionally substituted with one or more substituents independently selected from: halogen, —OR¹¹, —N(R¹⁴)₂, —C(O)R¹⁴, —NO₂, ═O, ═S, ═N(R¹⁴), and —CN; and C₁₋₆ alkyl optionally substituted with one or more substituents independently selected from halogen, —OR¹¹, —N(R¹⁴)₂, —C(O)R¹⁴, —NO₂, ═O, ═S, ═N(R¹⁴), and —CN; and R¹⁴ is independently selected at each occurrence from hydrogen, C₁₋₆ alkyl, and C₁₋₆ haloalkyl.

In some embodiments, one of R¹ or R² is selected from hydrogen and the other of R¹ or R² is selected from cyclopropyl, cyclobutyl, cyclopentyl, cyclohexyl, and tetrahydropyranyl, each of which is optionally substituted with one or more substituents independently selected from: halogen, —OR¹⁴, —N(R¹⁴)₂, —C(O)R¹⁴, —NO₂, ═O, ═S, ═N(R¹⁴), and —CN; and C₁₋₆ alkyl optionally substituted with one or more substituents independently selected from halogen, —OR¹⁴, —N(R¹⁴)₂, —C(O)R¹⁴, —NO₂, ═O, ═S, ═N(R¹⁴), and —CN; and R¹⁴ is independently selected at each occurrence from hydrogen, C₁₋₆ alkyl, and C₁₋₆ haloalkyl. In some embodiments, one of R¹ or R² is selected from hydrogen and the other of R¹ or R² is selected from:

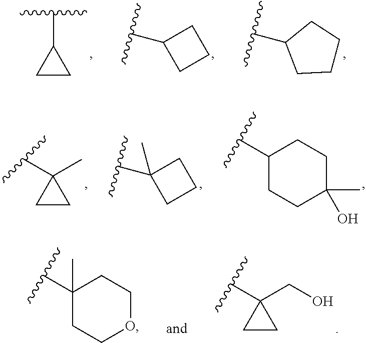

In some embodiments, one of R¹ or R² is selected from hydrogen and the other of R¹ or R² is selected from:

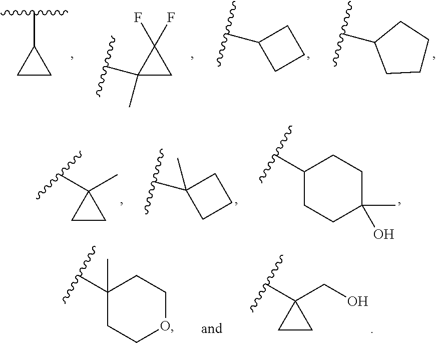

some embodiments, one of R¹ or R² is selected from hydrogen and the other of R¹ or R² is selected from:

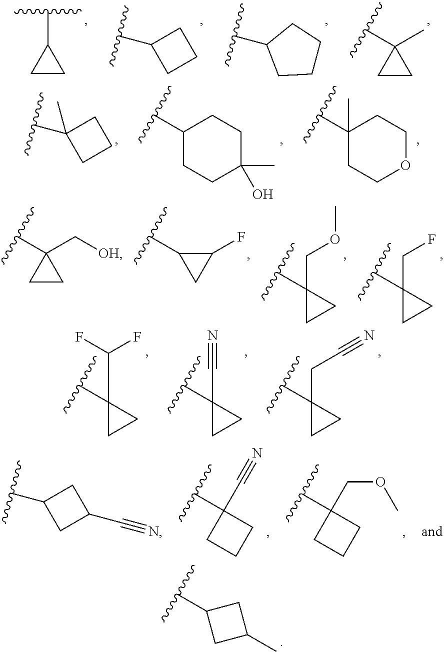

In some embodiments, one of R¹ or R² is selected from hydrogen and the other of R¹ or R² is selected from:

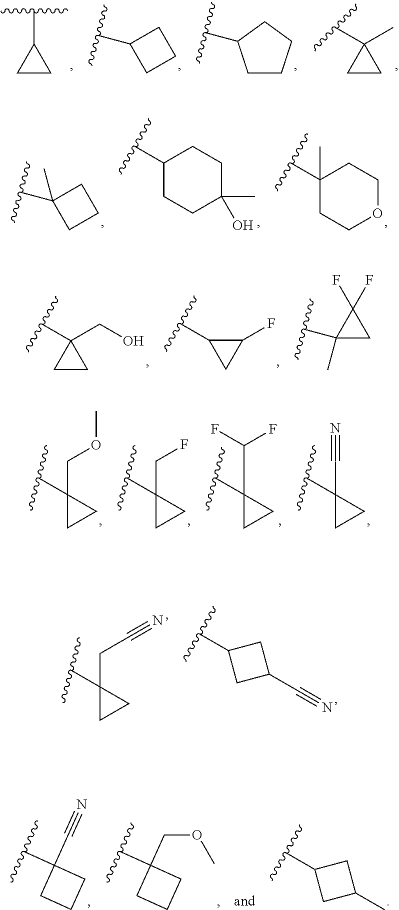

In some embodiments, one of R¹ or R² is selected from hydrogen and the other of R¹ or R² is selected from cyclopropyl, cyclobutyl, cyclopentyl, cyclohexyl, bicyclo[1.1.1]pentyl, oxetanyl, tetrahydropyranyl, azetidinyl, pyrrolidinyl, pyridyl, and phenyl, each of which is optionally substituted with one or more substituents independently selected from: halogen, —OR¹⁴, —N(R¹⁴)₂, —C(O)R¹⁴, —NO₂, ═O, ═S, ═N(R¹⁴), and —CN; and C₁₋₆ alkyl optionally substituted with one or more substituents independently selected from halogen, —OR⁴, —N(R¹⁴)₂, —C(O)R¹⁴, —NO₂, ═O, ═S, ═N(R¹⁴), and —CN; and R¹⁴ is independently selected at each occurrence from hydrogen, C₁₋₆ alkyl, and C₁₋₆ haloalkyl. In some embodiments, one of R¹ or R² is selected from hydrogen and the other of R¹ or R² is selected from:

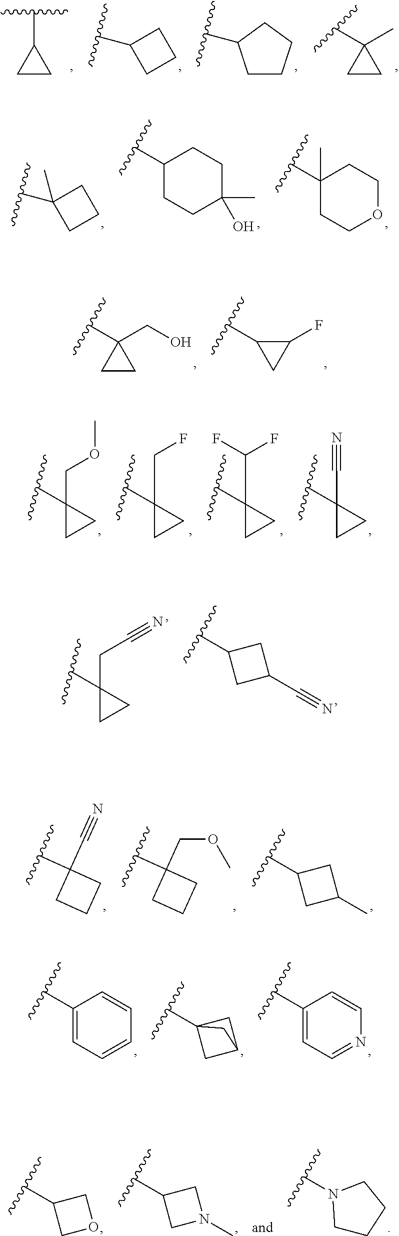

In some embodiments, one of R¹ or R² is selected from hydrogen and the other of R¹ or R² is selected from:

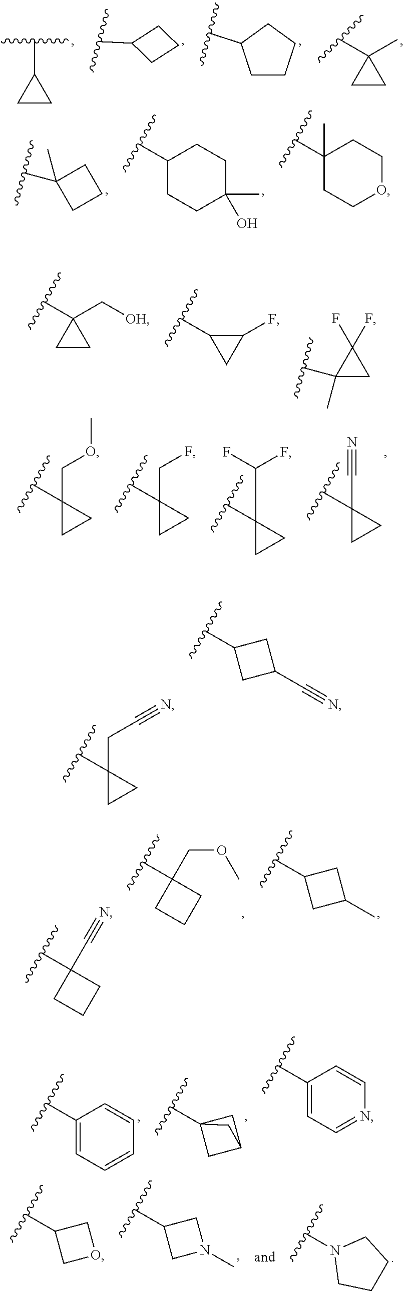

In some embodiments, for the compound or salt of Formula (I), (II), (II-A), (II-B), (II-C), (II-D) (II-E), (II-F), (III), (IV), (IV-A), (IV-B), (IV-C), or (V), one of R¹ or R² is selected from hydrogen and the other of R¹ or R² is selected from

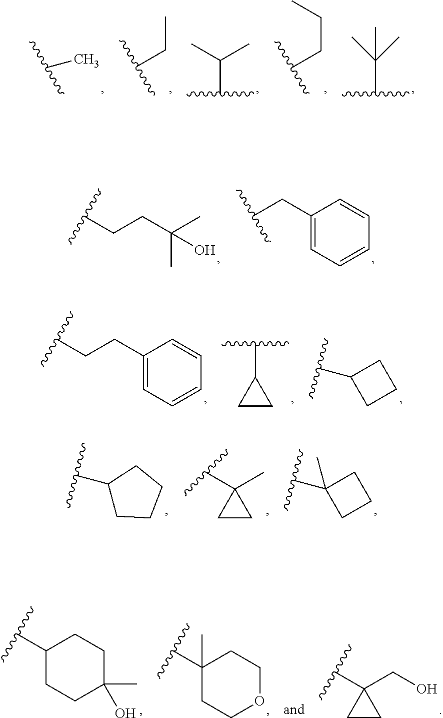

In some embodiments, for the compound or salt of Formula (I), (II), (II-A), (II-B), (II-C), (II-D) (II-E), (II-F), (III), (IV), (IV-A), (IV-B), (IV-C), or (V), one of R¹ or R² is selected from hydrogen and the other of R¹ or R² is selected from

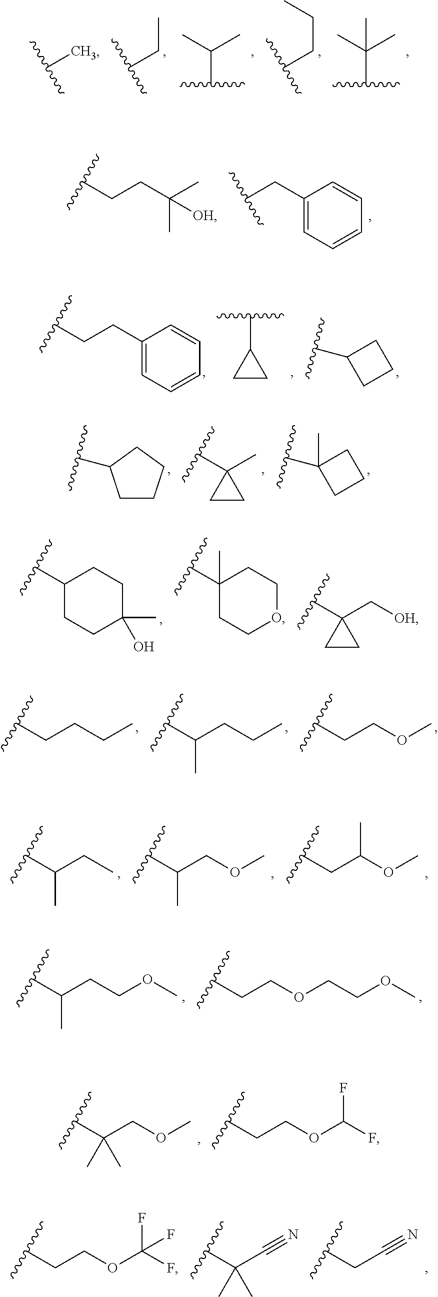

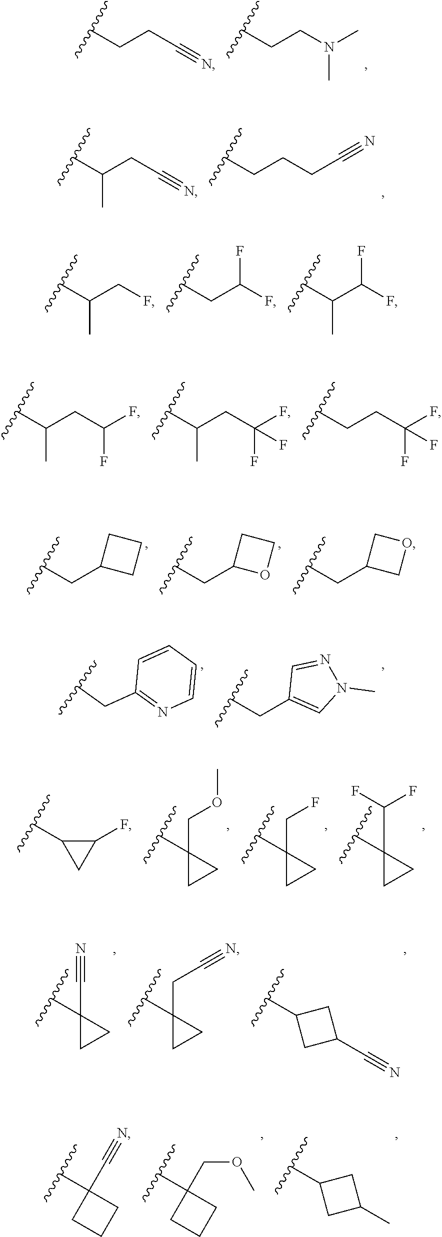

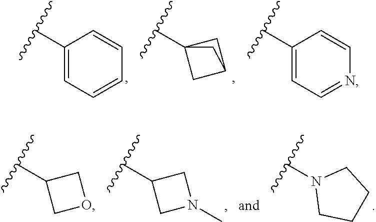

In some embodiments, for the compound or salt of Formula (I), (II), (II-A), (II-B), (II-C), (II-D) (II-E), (II-F), (III), (IV), (IV-A), (IV-B), (IV-C), or (V), one of R¹ or R² is selected from hydrogen and the other of R¹ or R² is selected from

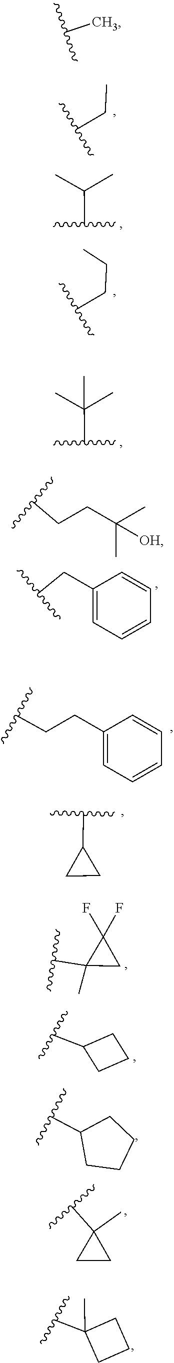

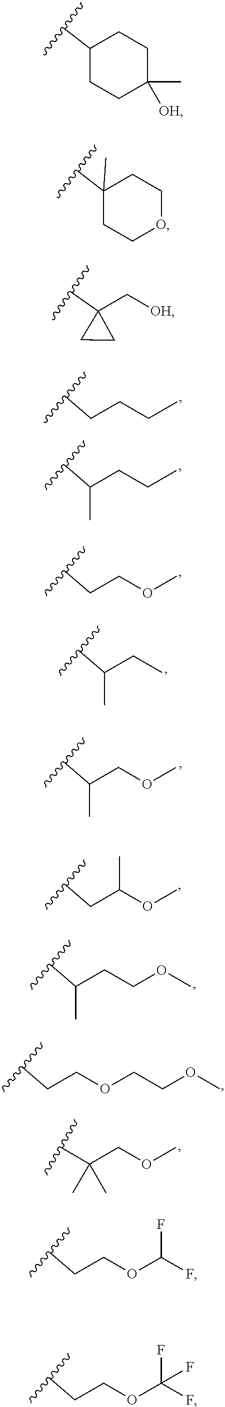

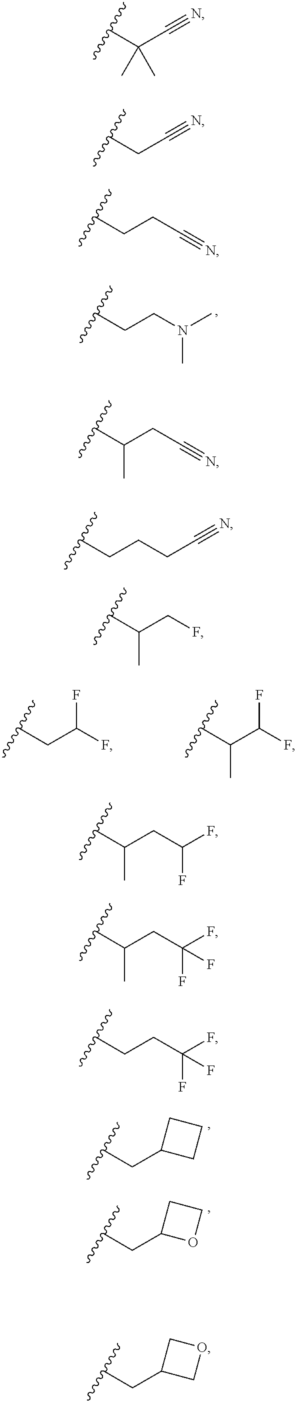

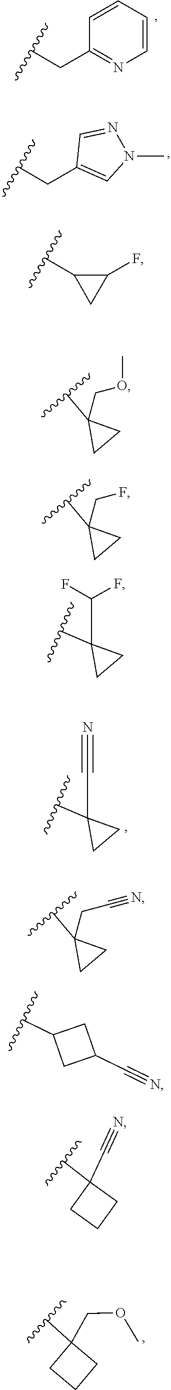

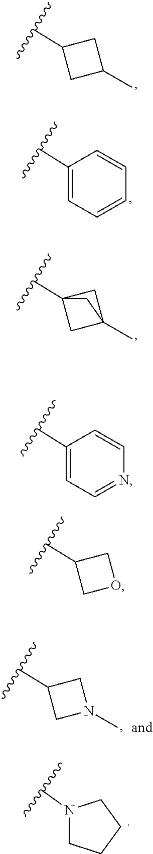

In some embodiments, for the compound or salt of Formula (I), (II), (II-A), (II-B), (II-C), (II-D), (II-E), (II-F), (III), (IV), (IV-A), (IV-B), (IV-C), or (V), R¹ and R² may come together to form a 3- to 10-membered heterocycle optionally substituted with one or more substituents independently selected from: halogen, —OR¹⁵, —SR¹⁵, —N(R¹⁵)₂, —C(O)R¹⁵, —C(O)N(R¹⁵)₂, —N(R¹)C(O)R¹⁵, —C(O)OR¹⁵, —OC(O)R¹⁵, —N(R¹)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R⁵)₂, —NO₂, ═O, ═S, ═N(R¹⁵), and —CN; and C₁₋₆ alkyl optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN. In some embodiments, R¹ and R² may come together to form a 3- to 6-membered saturated heterocycle optionally substituted with one or more substituents independently selected from halogen, —OR¹⁵, —N(R¹⁵)₂, —C(O)R¹⁵, —NO₂, ═O, ═S, ═N(R¹⁵), —CN; and C₁₋₆ alkyl optionally substituted with one or more substituents independently selected from halogen, —OR¹⁵, —N(R¹⁵)₂, —C(O)R¹⁵, —NO₂, ═O, ═S, ═N(R¹⁵), and —CN. In some embodiments, R¹ and R² may come together to form a 3- to 6-membered saturated heterocycle optionally substituted with one or more substituents independently selected from halogen, —OR¹⁵, —N(R¹⁵)₂, —C(O)R¹⁵, —NO₂, ═O, ═S, ═N(R¹⁵), —CN; and C₁₋₆ alkyl optionally substituted with one or more substituents independently selected from halogen, —OR¹⁵, —N(R¹⁵)₂, —C(O)R¹⁵, —NO₂, ═O, ═S, ═N(R¹⁵), and —CN; and R¹⁵ is independently selected at each occurrence from hydrogen, C₁₋₆ alkyl, and C₁₋₆ haloalkyl. In some embodiments,

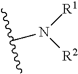

is selected from

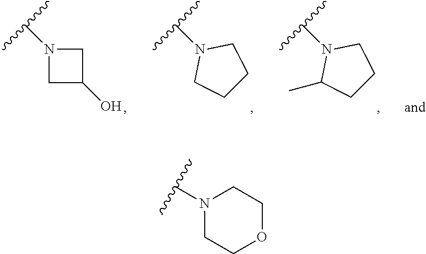

In some embodiments,

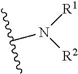

is selected from

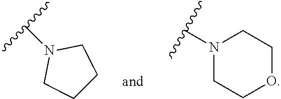 [SMILES: *N1CCCC1 ; *N1CCOCC1]

In some embodiments,

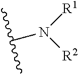

is selected from

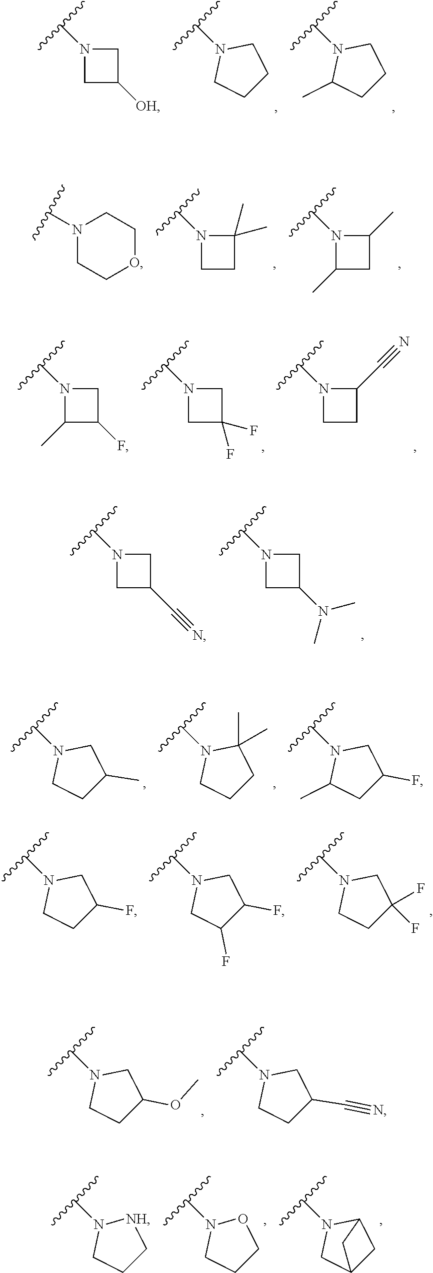

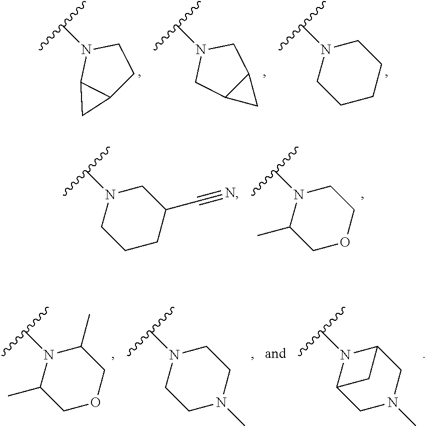

In some embodiments,

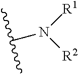

is selected from

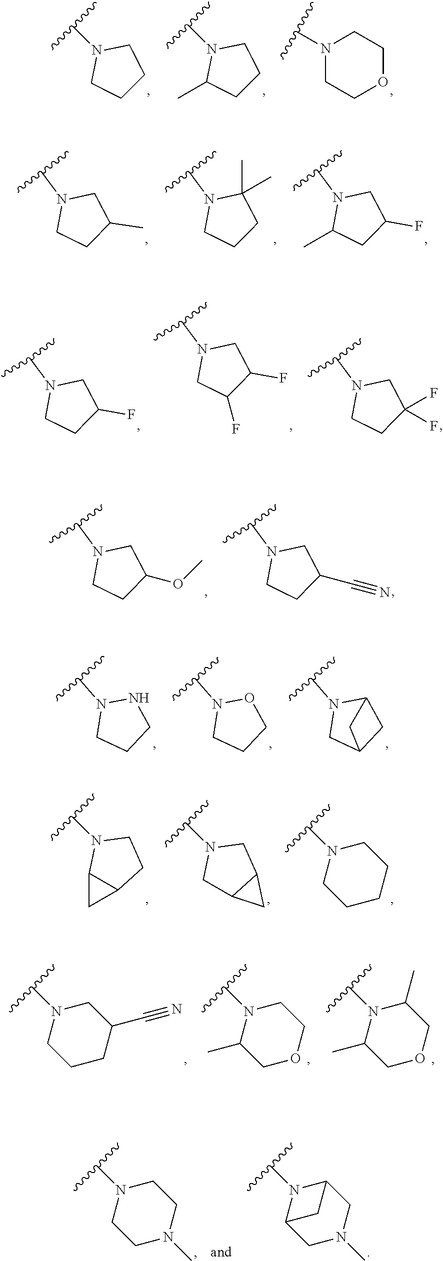

In some embodiments,

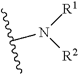

is selected from

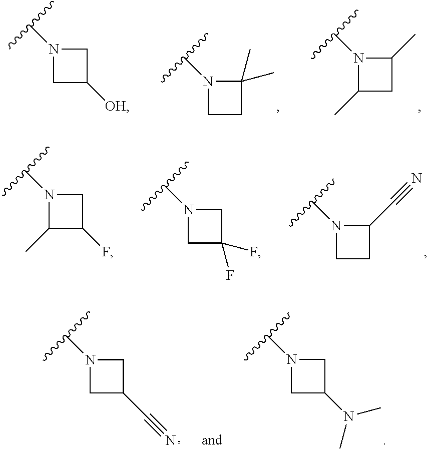

In some embodiments, R¹ and R² may come together to form a 3- to 8-membered saturated heterocycle optionally substituted with one or more substituents independently selected from halogen, —OR¹⁵, —N(R¹⁵)₂, —C(O)R¹⁵, —NO₂, ═O, ═S, ═N(R¹⁵), —CN; and C₁₋₆ alkyl optionally substituted with one or more substituents independently selected from halogen, —OR¹⁵, —N(R¹⁵)₂, —C(O)R¹⁵, —NO₂, ═O, ═S, ═N(R¹⁵), and —CN. In some embodiments, R¹ and R² may come together to form a 3- to 8-membered saturated heterocycle optionally substituted with one or more substituents independently selected from halogen, —OR¹⁵, —N(R¹⁵)₂, —C(O)R¹⁵, —NO₂, ═O, ═S, ═N(R¹⁵), —CN; and C₁₋₆ alkyl optionally substituted with one or more substituents independently selected from halogen, —OR¹⁵, —N(R¹⁵)₂, —C(O)R¹⁵, —NO₂, ═O, ═S, ═N(R¹⁵), and —CN; and R¹⁵ is independently selected at each occurrence from hydrogen, C₁₋₆ alkyl, and C₁₋₆ haloalkyl. In some embodiments,

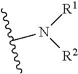

is selected from

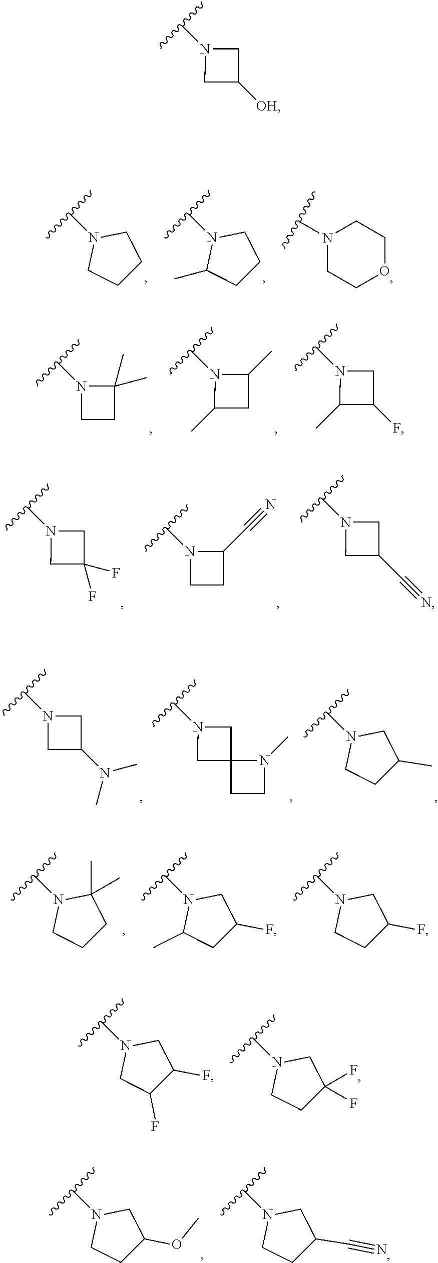

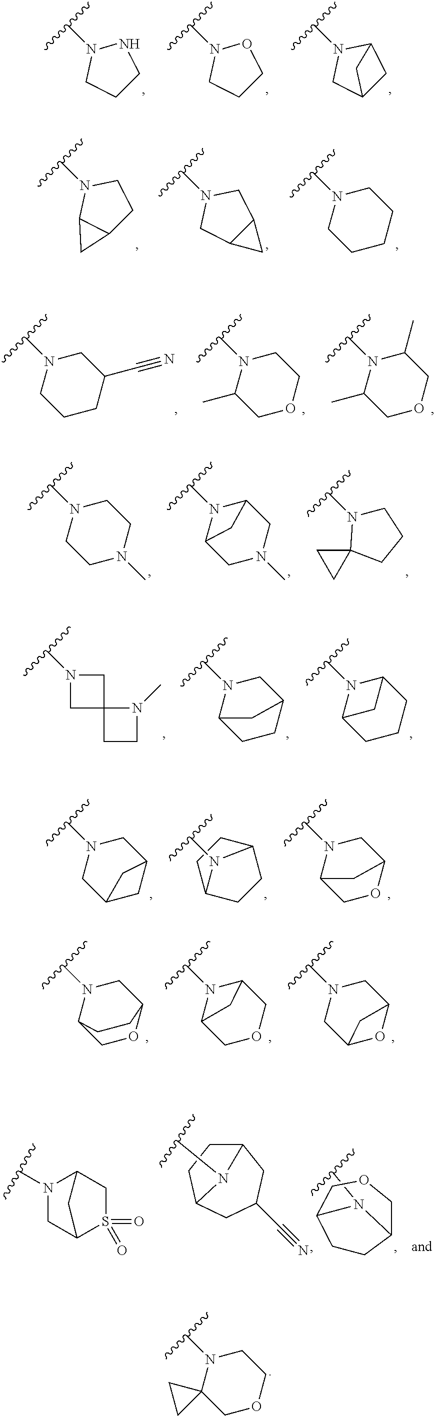

In some embodiments,

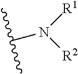

is selected from

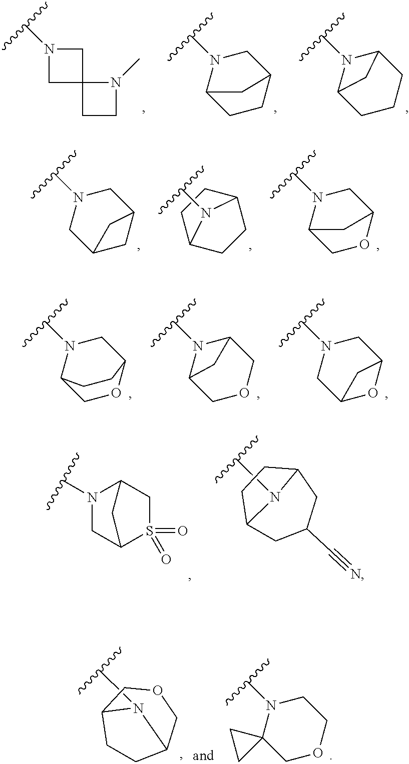

In some embodiments,

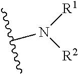

is selected from

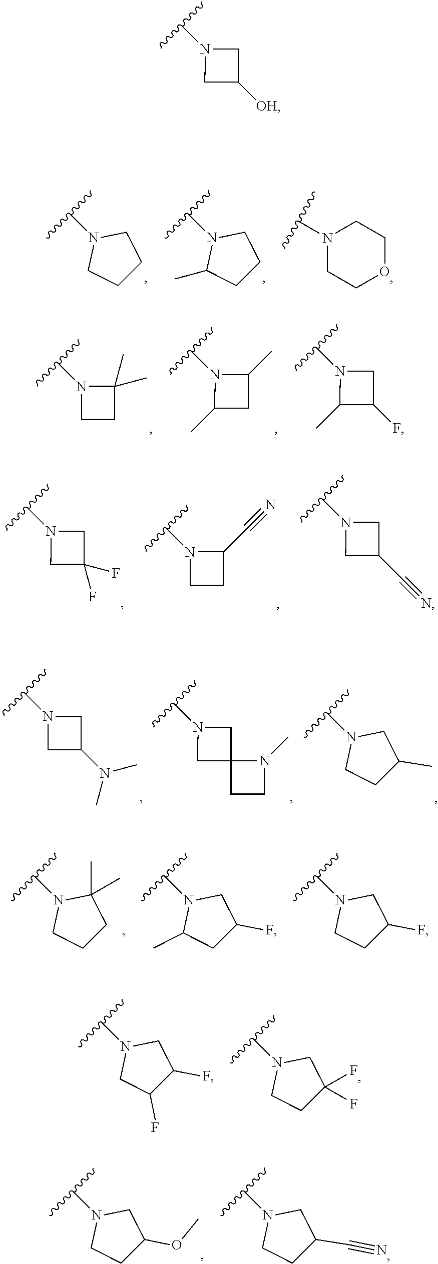

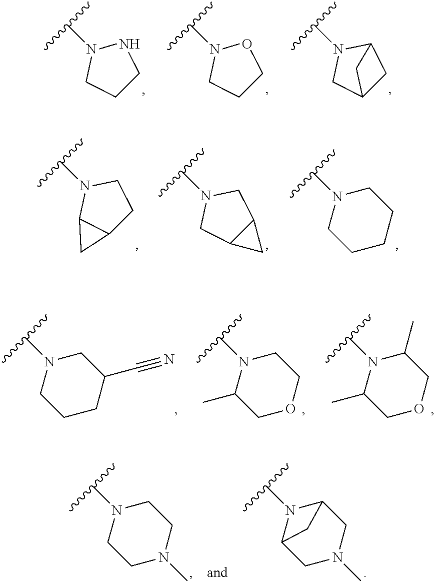

In some embodiments, for the compound or salt of Formula (I), (II), (II-A), (II-B), (II-C), (II-D), (II-E), (II-F), (III), (IV), (IV-A), (IV-B), (IV-C), or (V), R¹¹, R¹², R¹³, R¹⁴, R¹⁵, and R¹⁶ are each independently selected at each occurrence from hydrogen; C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₆ carbocycle, and 3- to 6-membered heterocycle; and C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN. In some embodiments, R¹¹, R¹², R¹³, R¹⁴, R¹⁵, and R¹⁶ are each independently selected at each occurrence from hydrogen, C₁₋₆ alkyl, C₁₋₆ haloalkyl, C₁₋₆alkyl-O—C₁₋₆ alkyl, C₁₋₆alkyl-O—C₁₋₆ haloalkyl, C₁₋₆alkyl-(C₃₋₆ carbocycle) and C₁₋₆ alkyl-(3- to 6-membered heterocycle), C₃₋₆ carbocycle, 3- to 6-membered heterocycle. In some embodiments, R¹¹, R¹², R¹³, R¹⁴, R, and R¹⁶ are each independently selected at each occurrence from hydrogen, C₁₋₆ alkyl, and C₁₋₆ haloalkyl.

In some aspects, for the compound or salt of Formula (I), (II), (II-A), (II-B), (II-C), (II-D), (II-E), (II-F), (III), (IV), (IV-A), (IV-B), (IV-C), or (V), Ring A is selected from C₃₋₆ carbocycle and 3- to 6-membered heterocycle.

In some aspects, for the compound or salt of Formula (I), (II), (II-A), (II-B), (II-C), (II-D), (II-E), (II-F), (III), (IV), (IV-A), (IV-B), (IV-C), or (V), n is selected from 0, 1, and 2.

In some aspects, for the compound or salt of Formula (I), (II), (II-A), (II-B), (II-C), (I-D), (II-E), (II-F), (III), (IV), (IV-A), (IV-B), (IV-C), or (V), R⁴ is selected from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

In some aspects, for the compound or salt of Formula (I), (II), (II-A), (II-B), (II-C), (II-D), (II-E), (II-F), (III), (IV), (IV-A), (IV-B), (IV-C), or (V), L is represented by -L¹¹-L²-L³-L⁴-, wherein L¹, L², L³, and L⁴ are each independently selected from (a) and (b):
(a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
(b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

In some aspects, for the compound or salt of Formula (I), (II), (II-A), (II-B), (II-C), (II-D), (II-E), (II-F), (III), (IV), (IV-A), (IV-B), (IV-C), or (V), R³ is selected from hydrogen and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —C(O)OR¹⁶, —NO₂, and —CN.

In some aspects, for the compound or salt of Formula (I), (II), (II-A), (II-B), (II-C), (II-D), (II-E), (II-F), (III), (IV), (IV-A), (IV-B), (IV-C), or (V), R¹ or R² is hydrogen.

In some aspects, for the compound or salt of Formula (I), (II), (II-A), (II-B), (II-C), (II-D), (II-E), (II-F), (III), (IV), (IV-A), (IV-B), (IV-C), or (V), R¹ or R² is selected from:
C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
C₃₋₆ carbocycle optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN, C₁₋₆ alkyl, and C₁₋₆ haloalkyl.

In some aspects, for the compound or salt of Formula (I), (II), (II-A), (II-B), (II-C), (II-D), (II-E), (II-F), (III), (IV), (IV-A), (IV-B), (IV-C), or (V), one of R¹ and R² is selected from hydrogen and the other of R¹ or R² is selected from:
C₁₋₆ alkyl optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
C₃₋₆ carbocycle optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN, C₁₋₆ alkyl, and C₁₋₆ haloalkyl.

In some embodiments, the compound or salt of Formula (I), (II), (II-A), (II-B), (II-C), (II-D), (II-E), (II-F), (III), (IV), (IV-A), (IV-B), (IV-C), or (V), is selected from a compound in Table 1.

TABLE 1
Selected CDK2 modulators. * Denotes a stereocenter with an undetermined absolute stereochemistry of a single isomer.

| # | Compound |
|---|---|
| 1 | 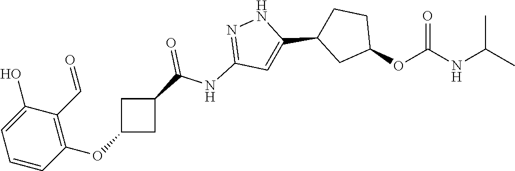 [SMILES: CC(C)NC(=O)O[C@@H]1CC[C@H](c2cc(NC(=O)[C@H]3C[C@H](Oc4cccc(O)c4C=O)C3)n[nH]2)C1] |
| 2 | 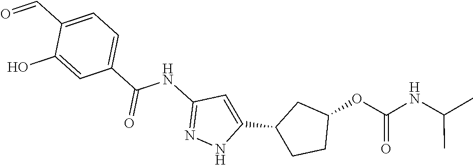 [SMILES: CC(C)NC(=O)O[C@@H]1CC[C@H](c2cc(NC(=O)c3ccc(C=O)c(O)c3)n[nH]2)C1] |
| 3 | 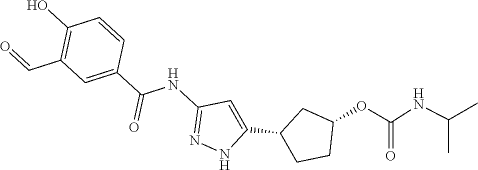 [SMILES: CC(C)NC(=O)O[C@@H]1CC[C@H](c2cc(NC(=O)c3ccc(O)c(C=O)c3)n[nH]2)C1] |
| 4 | 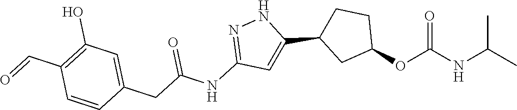 [SMILES: CC(C)NC(=O)O[C@@H]1CC[C@H](c2cc(NC(=O)Cc3ccc(C=O)c(O)c3)n[nH]2)C1] |
| 5 | 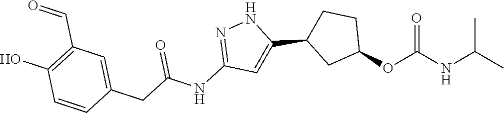 [SMILES: CC(C)NC(=O)O[C@@H]1CC[C@H](c2cc(NC(=O)Cc3ccc(O)c(C=O)c3)n[nH]2)C1] |
| 6 | 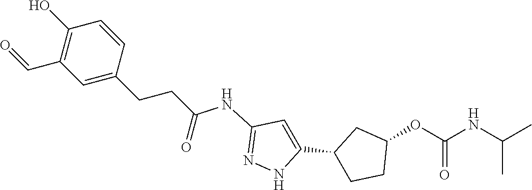 [SMILES: CC(C)NC(=O)O[C@@H]1CC[C@H](c2cc(NC(=O)CCc3ccc(O)c(C=O)c3)n[nH]2)C1] |
| 7 | 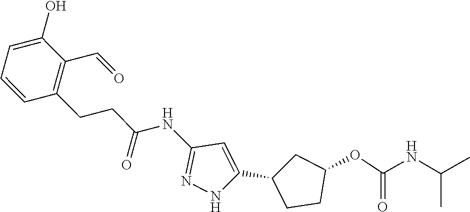 [SMILES: CC(C)NC(=O)O[C@@H]1CC[C@H](c2cc(NC(=O)CCc3cccc(O)c3C=O)n[nH]2)C1] |
| 8 | 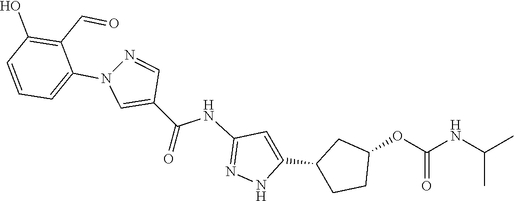 [SMILES: CC(C)NC(=O)O[C@@H]1CC[C@H](c2cc(NC(=O)c3cnn(-c4cccc(O)c4C=O)c3)n[nH]2)C1] |
| 9 | 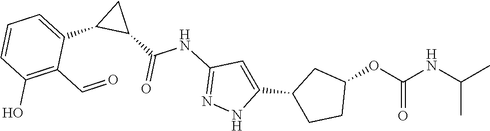 [SMILES: CC(C)NC(=O)O[C@@H]1CC[C@H](c2cc(NC(=O)[C@H]3C[C@H]3c3cccc(O)c3C=O)n[nH]2)C1] |
| 10 | 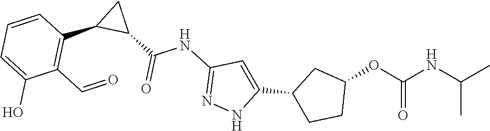 [SMILES: CC(C)NC(=O)O[C@@H]1CC[C@H](c2cc(NC(=O)[C@H]3C[C@@H]3c3cccc(O)c3C=O)n[nH]2)C1] |
| 11 | 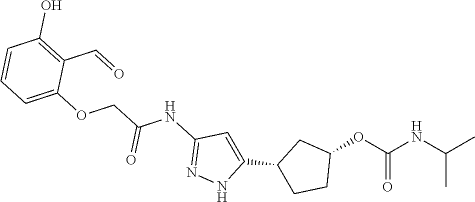 [SMILES: CC(C)NC(=O)O[C@@H]1CC[C@H](c2cc(NC(=O)COc3cccc(O)c3C=O)n[nH]2)C1] |
| 12 | 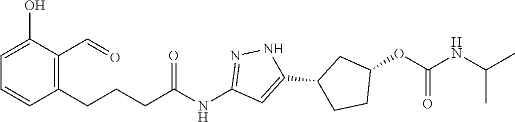 [SMILES: CC(C)NC(=O)O[C@@H]1CC[C@H](c2cc(NC(=O)CCCc3cccc(O)c3C=O)n[nH]2)C1] |
| 13 | 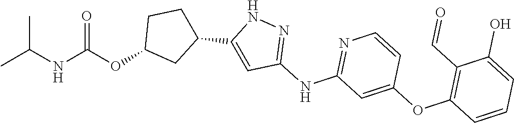 [SMILES: CC(C)NC(=O)O[C@@H]1CC[C@H](c2cc(Nc3cc(Oc4cccc(O)c4C=O)ccn3)n[nH]2)C1] |
| 14 | 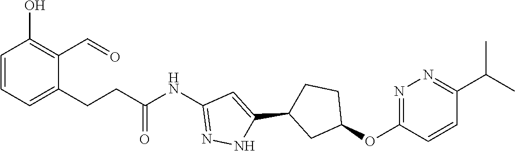 [SMILES: CC(C)c1ccc(O[C@@H]2CC[C@H](c3cc(NC(=O)CCc4cccc(O)c4C=O)n[nH]3)C2)nn1] |
| 15 | 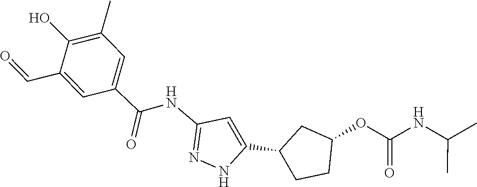 [SMILES: Cc1cc(C(=O)Nc2cc([C@H]3CC[C@@H](OC(=O)NC(C)C)C3)[nH]n2)cc(C=O)c1O] |
| 16 | 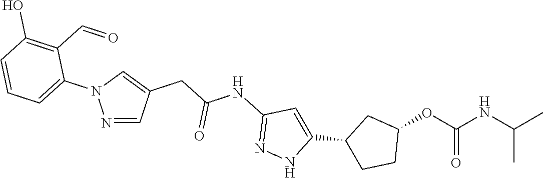 [SMILES: CC(C)NC(=O)O[C@@H]1CC[C@H](c2cc(NC(=O)Cc3cnn(-c4cccc(O)c4C=O)c3)n[nH]2)C1] |
| 17 | 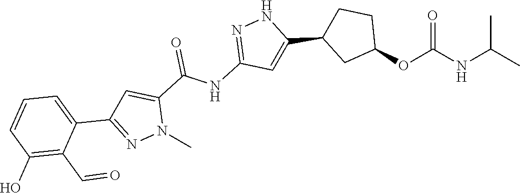 [SMILES: CC(C)NC(=O)O[C@@H]1CC[C@H](c2cc(NC(=O)c3cc(-c4cccc(O)c4C=O)nn3C)n[nH]2)C1] |
| 18 | 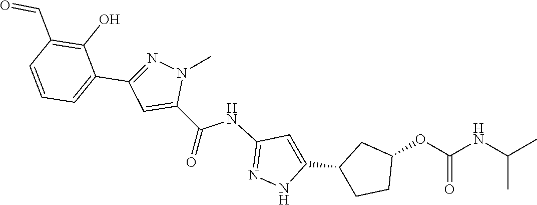 [SMILES: CC(C)NC(=O)O[C@@H]1CC[C@H](c2cc(NC(=O)c3cc(-c4cccc(C=O)c4O)nn3C)n[nH]2)C1] |
| 19 | 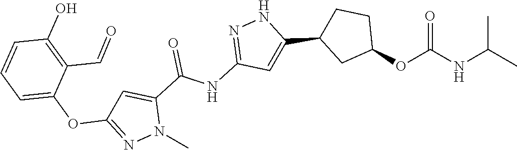 [SMILES: CC(C)NC(=O)O[C@@H]1CC[C@H](c2cc(NC(=O)c3cc(Oc4cccc(O)c4C=O)nn3C)n[nH]2)C1] |
| 20 | 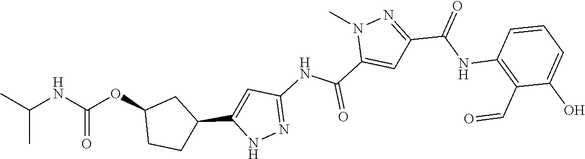 [SMILES: CC(C)NC(=O)O[C@@H]1CC[C@H](c2cc(NC(=O)c3cc(C(=O)Nc4cccc(O)c4C=O)nn3C)n[nH]2)C1] |
| 21 | 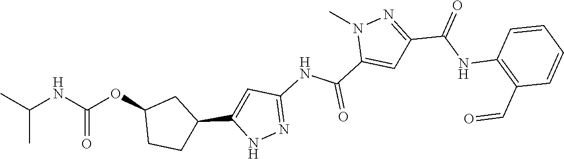 [SMILES: CC(C)NC(=O)O[C@@H]1CC[C@H](c2cc(NC(=O)c3cc(C(=O)Nc4ccccc4C=O)nn3C)n[nH]2)C1] |
| 22 | 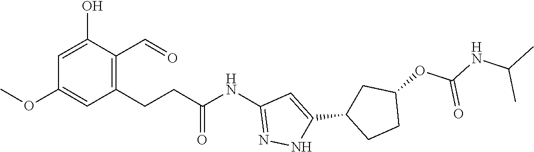 [SMILES: COc1cc(O)c(C=O)c(CCC(=O)Nc2cc([C@H]3CC[C@@H](OC(=O)NC(C)C)C3)[nH]n2)c1] |
| 23 | 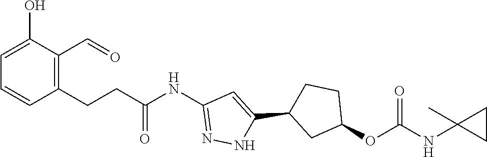 [SMILES: CC1(NC(=O)O[C@@H]2CC[C@H](c3cc(NC(=O)CCc4cccc(O)c4C=O)n[nH]3)C2)CC1] |
| 24 | 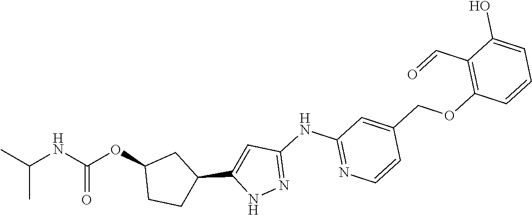 [SMILES: CC(C)NC(=O)O[C@@H]1CC[C@H](c2cc(Nc3cc(COc4cccc(O)c4C=O)ccn3)n[nH]2)C1] |
| 25 | 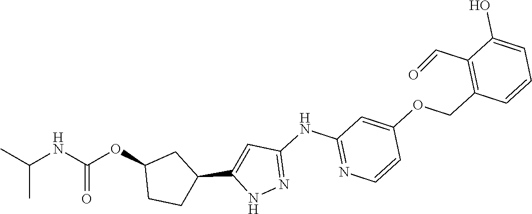 [SMILES: CC(C)NC(=O)O[C@@H]1CC[C@H](c2cc(Nc3cc(OCc4cccc(O)c4C=O)ccn3)n[nH]2)C1] |
| 26 | 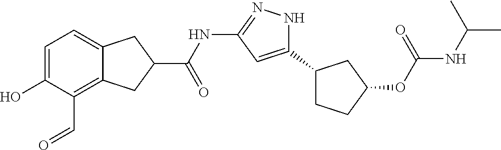 [SMILES: CC(C)NC(=O)O[C@@H]1CC[C@H](c2cc(NC(=O)C3Cc4ccc(O)c(C=O)c4C3)n[nH]2)C1] |
| 27 | 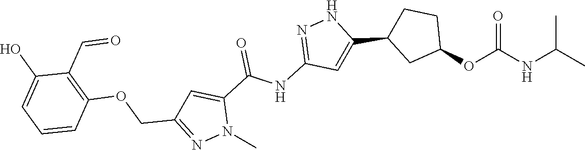 [SMILES: CC(C)NC(=O)O[C@@H]1CC[C@H](c2cc(NC(=O)c3cc(COc4cccc(O)c4C=O)nn3C)n[nH]2)C1] |
| 28 | 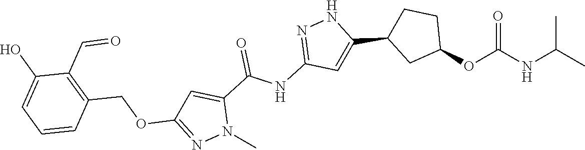 [SMILES: CC(C)NC(=O)O[C@@H]1CC[C@H](c2cc(NC(=O)c3cc(OCc4cccc(O)c4C=O)nn3C)n[nH]2)C1] |
| 29 | 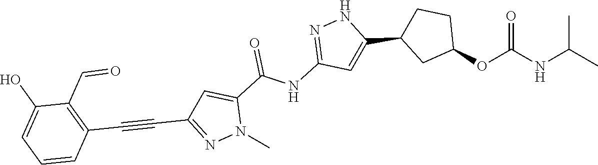 [SMILES: CC(C)NC(=O)O[C@@H]1CC[C@H](c2cc(NC(=O)c3cc(C#Cc4cccc(O)c4C=O)nn3C)n[nH]2)C1] |
| 30 | 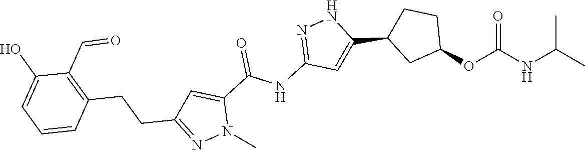 [SMILES: CC(C)NC(=O)O[C@@H]1CC[C@H](c2cc(NC(=O)c3cc(CCc4cccc(O)c4C=O)nn3C)n[nH]2)C1] |
| 31 | 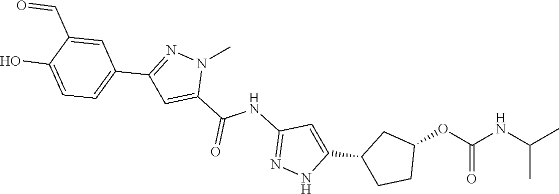 [SMILES: CC(C)NC(=O)O[C@@H]1CC[C@H](c2cc(NC(=O)c3cc(-c4ccc(O)c(C=O)c4)nn3C)n[nH]2)C1] |
| 32 | 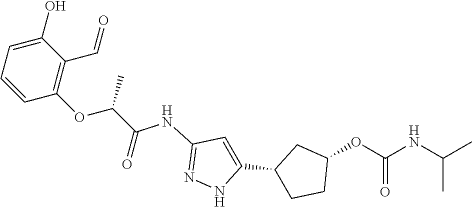 [SMILES: CC(C)NC(=O)O[C@@H]1CC[C@H](c2cc(NC(=O)[C@@H](C)Oc3cccc(O)c3C=O)n[nH]2)C1] |
| 33 | 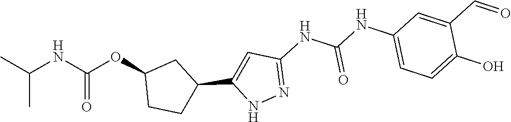 [SMILES: CC(C)NC(=O)O[C@@H]1CC[C@H](c2cc(NC(=O)Nc3ccc(O)c(C=O)c3)n[nH]2)C1] |
| 34 | 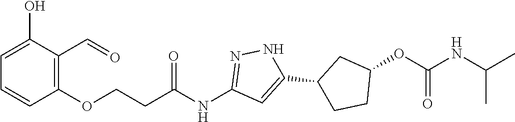 [SMILES: CC(C)NC(=O)O[C@@H]1CC[C@H](c2cc(NC(=O)CCOc3cccc(O)c3C=O)n[nH]2)C1] |
| 35 | 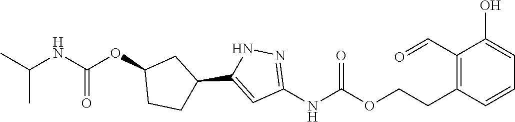 [SMILES: CC(C)NC(=O)O[C@@H]1CC[C@H](c2cc(NC(=O)OCCc3cccc(O)c3C=O)n[nH]2)C1] |
| 36 | 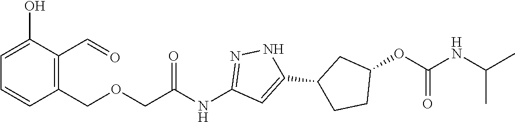 [SMILES: CC(C)NC(=O)O[C@@H]1CC[C@H](c2cc(NC(=O)COCc3cccc(O)c3C=O)n[nH]2)C1] |
| 37 | 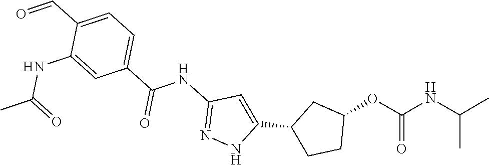 [SMILES: CC(=O)Nc1cc(C(=O)Nc2cc([C@H]3CC[C@@H](OC(=O)NC(C)C)C3)[nH]n2)ccc1C=O] |
| 38 | 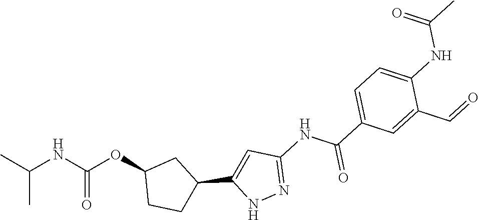 [SMILES: CC(=O)Nc1ccc(C(=O)Nc2cc([C@H]3CC[C@@H](OC(=O)NC(C)C)C3)[nH]n2)cc1C=O] |
| 39 | 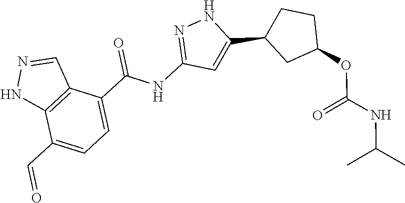 [SMILES: CC(C)NC(=O)O[C@@H]1CC[C@H](c2cc(NC(=O)c3ccc(C=O)c4[nH]ncc34)n[nH]2)C1] |
| 40 | 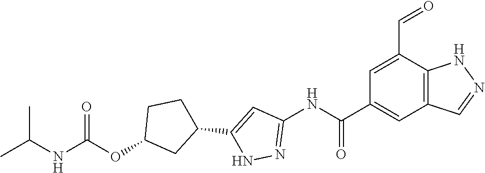 [SMILES: CC(C)NC(=O)O[C@@H]1CC[C@H](c2cc(NC(=O)c3cc(C=O)c4[nH]ncc4c3)n[nH]2)C1] |
| 41 | 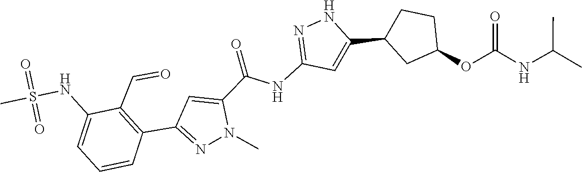 [SMILES: CC(C)NC(=O)O[C@@H]1CC[C@H](c2cc(NC(=O)c3cc(-c4cccc(NS(C)(=O)=O)c4C=O)nn3C)n[nH]2)C1] |
| 42 | 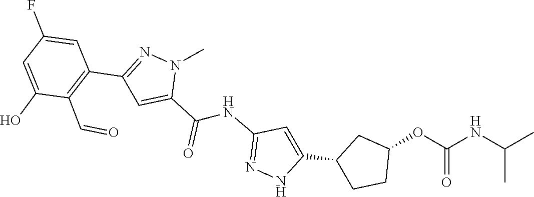 [SMILES: CC(C)NC(=O)O[C@@H]1CC[C@H](c2cc(NC(=O)c3cc(-c4cc(F)cc(O)c4C=O)nn3C)n[nH]2)C1] |
| 43 | 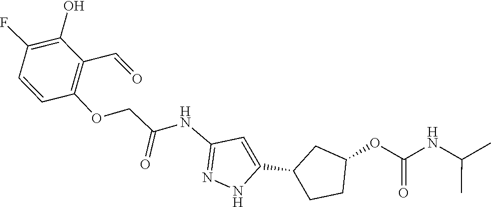 [SMILES: CC(C)NC(=O)O[C@@H]1CC[C@H](c2cc(NC(=O)COc3ccc(F)c(O)c3C=O)n[nH]2)C1] |
| 44 | 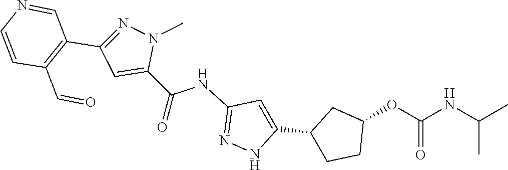 [SMILES: CC(C)NC(=O)O[C@@H]1CC[C@H](c2cc(NC(=O)c3cc(-c4cnccc4C=O)nn3C)n[nH]2)C1] |
| 45 | 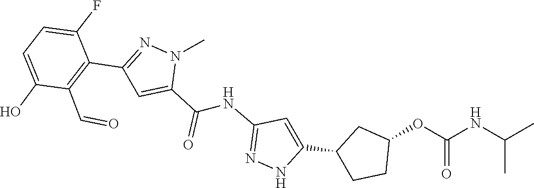 [SMILES: CC(C)NC(=O)O[C@@H]1CC[C@H](c2cc(NC(=O)c3cc(-c4c(F)ccc(O)c4C=O)nn3C)n[nH]2)C1] |
| 46 | 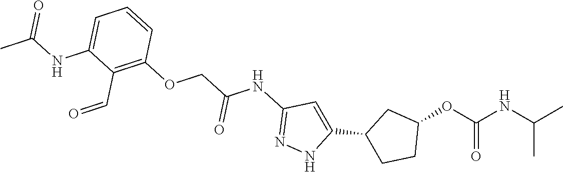 [SMILES: CC(=O)Nc1cccc(OCC(=O)Nc2cc([C@H]3CC[C@@H](OC(=O)NC(C)C)C3)[nH]n2)c1C=O] |
| 47 | 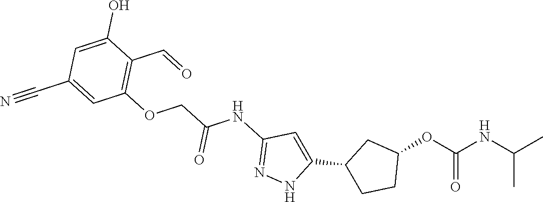 [SMILES: CC(C)NC(=O)O[C@@H]1CC[C@H](c2cc(NC(=O)COc3cc(C#N)cc(O)c3C=O)n[nH]2)C1] |
| 48 | 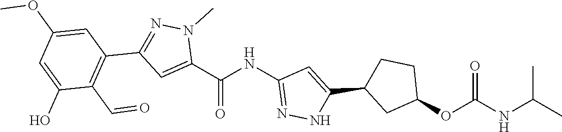 [SMILES: COc1cc(O)c(C=O)c(-c2cc(C(=O)Nc3cc([C@H]4CC[C@@H](OC(=O)NC(C)C)C4)[nH]n3)n(C)n2)c1] |
| 49 | 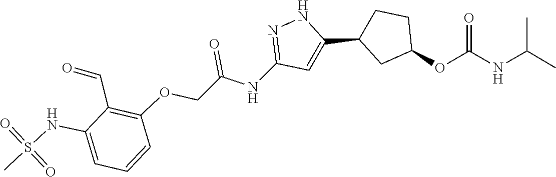 [SMILES: CC(C)NC(=O)O[C@@H]1CC[C@H](c2cc(NC(=O)COc3cccc(NS(C)(=O)=O)c3C=O)n[nH]2)C1] |
| 50 | 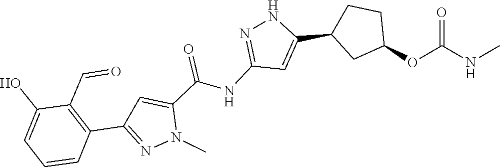 [SMILES: CNC(=O)O[C@@H]1CC[C@H](c2cc(NC(=O)c3cc(-c4cccc(O)c4C=O)nn3C)n[nH]2)C1] |
| 51 | 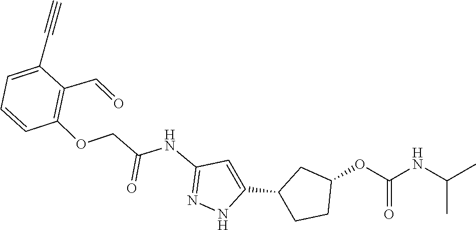 [SMILES: C#Cc1cccc(OCC(=O)Nc2cc([C@H]3CC[C@@H](OC(=O)NC(C)C)C3)[nH]n2)c1C=O] |
| 52 | 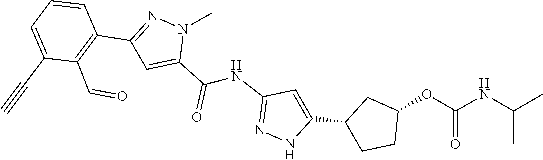 [SMILES: C#Cc1cccc(-c2cc(C(=O)Nc3cc([C@H]4CC[C@@H](OC(=O)NC(C)C)C4)[nH]n3)n(C)n2)c1C=O] |
| 53 | 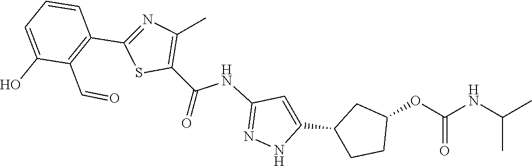 [SMILES: Cc1nc(-c2cccc(O)c2C=O)sc1C(=O)Nc1cc([C@H]2CC[C@@H](OC(=O)NC(C)C)C2)[nH]n1] |
| 54 | 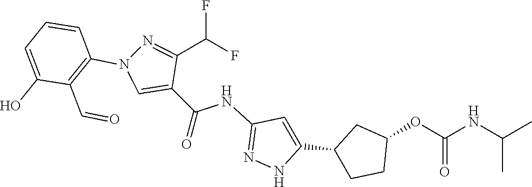 [SMILES: CC(C)NC(=O)O[C@@H]1CC[C@H](c2cc(NC(=O)c3cn(-c4cccc(O)c4C=O)nc3C(F)F)n[nH]2)C1] |
| 55 | 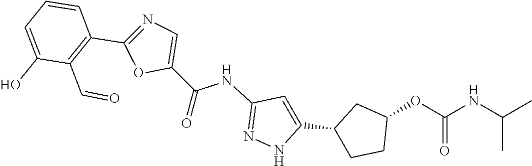 [SMILES: CC(C)NC(=O)O[C@@H]1CC[C@H](c2cc(NC(=O)c3cnc(-c4cccc(O)c4C=O)o3)n[nH]2)C1] |
| 56 | 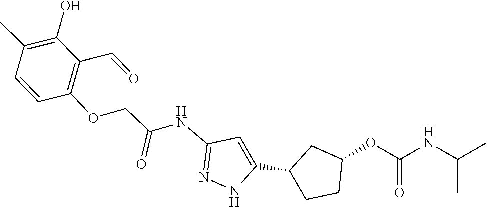 [SMILES: Cc1ccc(OCC(=O)Nc2cc([C@H]3CC[C@@H](OC(=O)NC(C)C)C3)[nH]n2)c(C=O)c1O] |
| 57 | 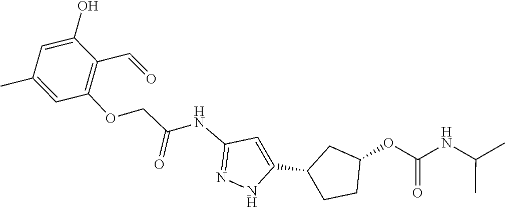 [SMILES: Cc1cc(O)c(C=O)c(OCC(=O)Nc2cc([C@H]3CC[C@@H](OC(=O)NC(C)C)C3)[nH]n2)c1] |
| 58 | 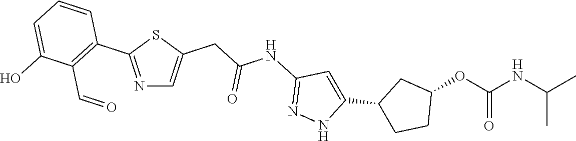 [SMILES: CC(C)NC(=O)O[C@@H]1CC[C@H](c2cc(NC(=O)Cc3cnc(-c4cccc(O)c4C=O)s3)n[nH]2)C1] |
| 59 | 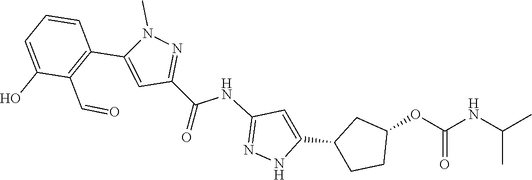 [SMILES: CC(C)NC(=O)O[C@@H]1CC[C@H](c2cc(NC(=O)c3cc(-c4cccc(O)c4C=O)n(C)n3)n[nH]2)C1] |
| 60 | 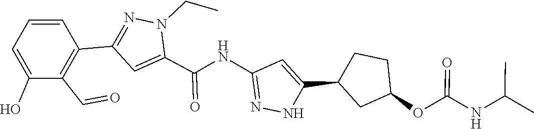 [SMILES: CCn1nc(-c2cccc(O)c2C=O)cc1C(=O)Nc1cc([C@H]2CC[C@@H](OC(=O)NC(C)C)C2)[nH]n1] |
| 61 | 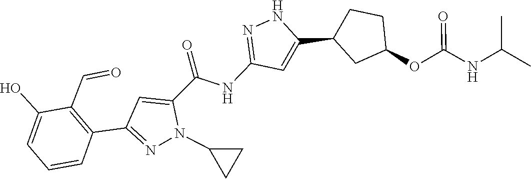 [SMILES: CC(C)NC(=O)O[C@@H]1CC[C@H](c2cc(NC(=O)c3cc(-c4cccc(O)c4C=O)nn3C3CC3)n[nH]2)C1] |
| 62 | 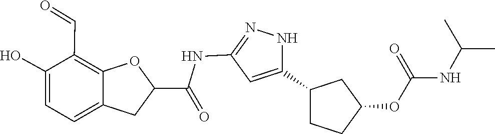 [SMILES: CC(C)NC(=O)O[C@@H]1CC[C@H](c2cc(NC(=O)C3Cc4ccc(O)c(C=O)c4O3)n[nH]2)C1] |
| 63 | 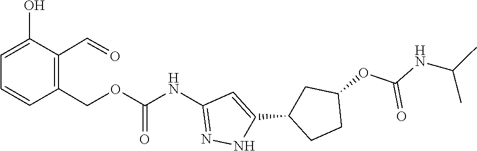 [SMILES: CC(C)NC(=O)O[C@@H]1CC[C@H](c2cc(NC(=O)OCc3cccc(O)c3C=O)n[nH]2)C1] |
| 64 | 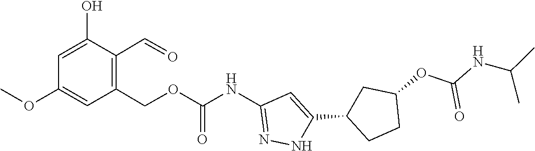 [SMILES: COc1cc(O)c(C=O)c(COC(=O)Nc2cc([C@H]3CC[C@@H](OC(=O)NC(C)C)C3)[nH]n2)c1] |
| 65 | 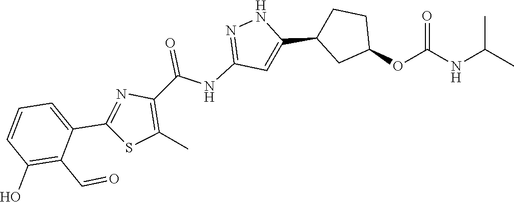 [SMILES: Cc1sc(-c2cccc(O)c2C=O)nc1C(=O)Nc1cc([C@H]2CC[C@@H](OC(=O)NC(C)C)C2)[nH]n1] |
| 66 | 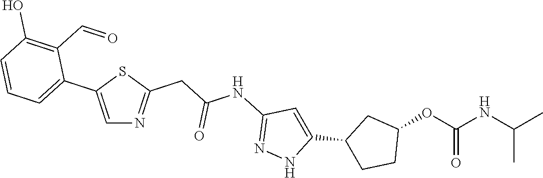 [SMILES: CC(C)NC(=O)O[C@@H]1CC[C@H](c2cc(NC(=O)Cc3ncc(-c4cccc(O)c4C=O)s3)n[nH]2)C1] |
| 67 | 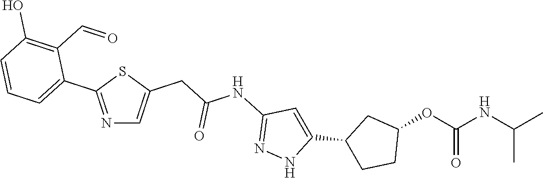 [SMILES: CC(C)NC(=O)O[C@@H]1CC[C@H](c2cc(NC(=O)Cc3cnc(-c4cccc(O)c4C=O)s3)n[nH]2)C1] |
| 68 | 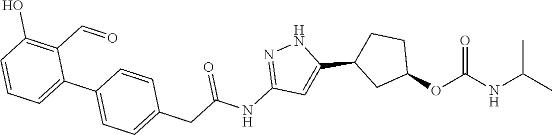 [SMILES: CC(C)NC(=O)O[C@@H]1CC[C@H](c2cc(NC(=O)Cc3ccc(-c4cccc(O)c4C=O)cc3)n[nH]2)C1] |
| 69 | 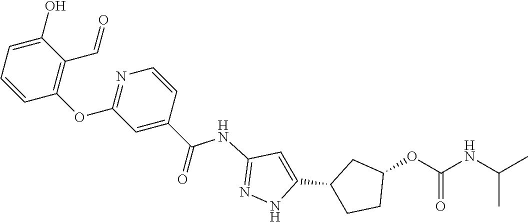 [SMILES: CC(C)NC(=O)O[C@@H]1CC[C@H](c2cc(NC(=O)c3ccnc(Oc4cccc(O)c4C=O)c3)n[nH]2)C1] |
| 70 | 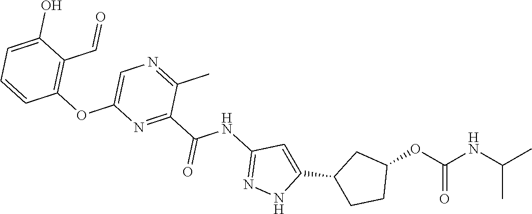 [SMILES: Cc1ncc(Oc2cccc(O)c2C=O)nc1C(=O)Nc1cc([C@H]2CC[C@@H](OC(=O)NC(C)C)C2)[nH]n1] |
| 71 | 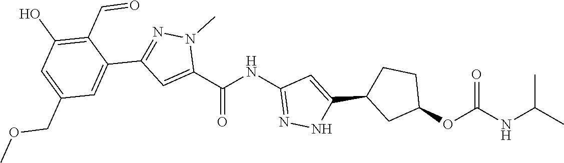 [SMILES: COCc1cc(O)c(C=O)c(-c2cc(C(=O)Nc3cc([C@H]4CC[C@@H](OC(=O)NC(C)C)C4)[nH]n3)n(C)n2)c1] |
| 72 | 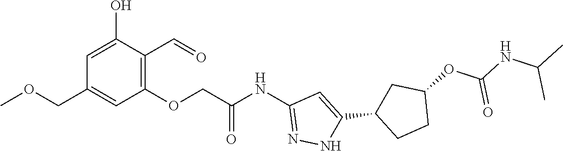 [SMILES: COCc1cc(O)c(C=O)c(OCC(=O)Nc2cc([C@H]3CC[C@@H](OC(=O)NC(C)C)C3)[nH]n2)c1] |
| 73 | 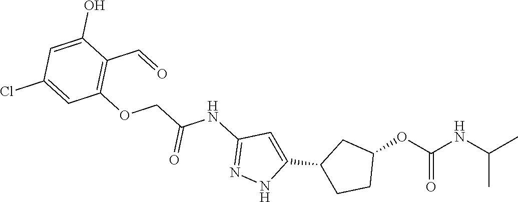 [SMILES: CC(C)NC(=O)O[C@@H]1CC[C@H](c2cc(NC(=O)COc3cc(Cl)cc(O)c3C=O)n[nH]2)C1] |
| 74 | 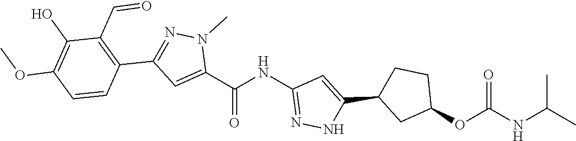 [SMILES: COc1ccc(-c2cc(C(=O)Nc3cc([C@H]4CC[C@@H](OC(=O)NC(C)C)C4)[nH]n3)n(C)n2)c(C=O)c1O] |
| 75 | 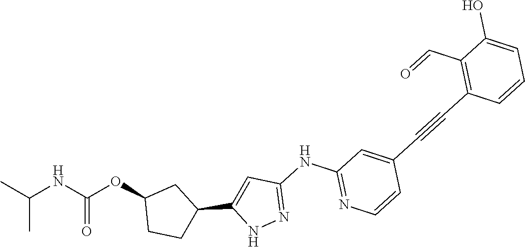 [SMILES: CC(C)NC(=O)O[C@@H]1CC[C@H](c2cc(Nc3cc(C#Cc4cccc(O)c4C=O)ccn3)n[nH]2)C1] |
| 76 | 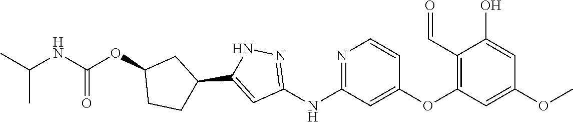 [SMILES: COc1cc(O)c(C=O)c(Oc2ccnc(Nc3cc([C@H]4CC[C@@H](OC(=O)NC(C)C)C4)[nH]n3)c2)c1] |
| 77 | 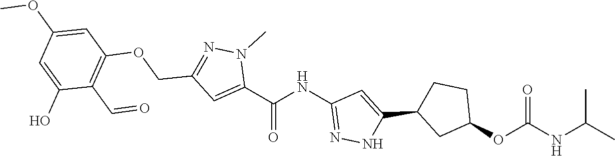 [SMILES: COc1cc(O)c(C=O)c(OCc2cc(C(=O)Nc3cc([C@H]4CC[C@@H](OC(=O)NC(C)C)C4)[nH]n3)n(C)n2)c1] |
| 78 | 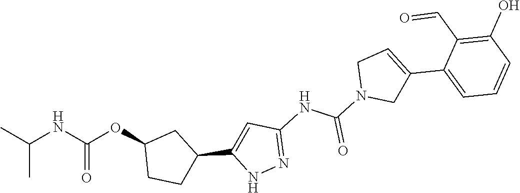 [SMILES: CC(C)NC(=O)O[C@@H]1CC[C@H](c2cc(NC(=O)N3CC=C(c4cccc(O)c4C=O)C3)n[nH]2)C1] |
| 79 | 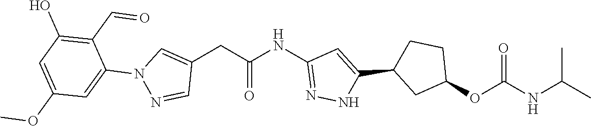 [SMILES: COc1cc(O)c(C=O)c(-n2cc(CC(=O)Nc3cc([C@H]4CC[C@@H](OC(=O)NC(C)C)C4)[nH]n3)cn2)c1] |
| 80 | 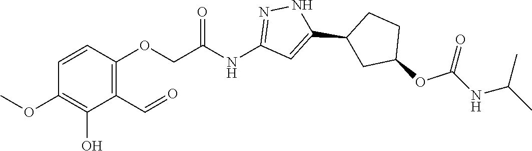 [SMILES: COc1ccc(OCC(=O)Nc2cc([C@H]3CC[C@@H](OC(=O)NC(C)C)C3)[nH]n2)c(C=O)c1O] |
| 81 | 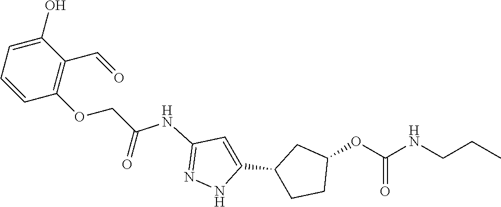 [SMILES: CCCNC(=O)O[C@@H]1CC[C@H](c2cc(NC(=O)COc3cccc(O)c3C=O)n[nH]2)C1] |
| 82 | 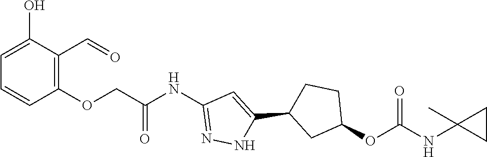 [SMILES: CC1(NC(=O)O[C@@H]2CC[C@H](c3cc(NC(=O)COc4cccc(O)c4C=O)n[nH]3)C2)CC1] |
| 83 | 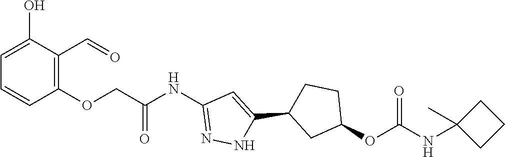 [SMILES: CC1(NC(=O)O[C@@H]2CC[C@H](c3cc(NC(=O)COc4cccc(O)c4C=O)n[nH]3)C2)CCC1] |
| 84 | 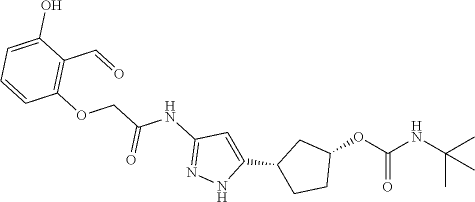 [SMILES: CC(C)(C)NC(=O)O[C@@H]1CC[C@H](c2cc(NC(=O)COc3cccc(O)c3C=O)n[nH]2)C1] |
| 85 | 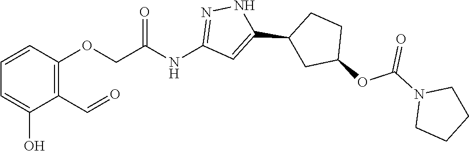 [SMILES: O=Cc1c(O)cccc1OCC(=O)Nc1cc([C@H]2CC[C@@H](OC(=O)N3CCCC3)C2)[nH]n1] |
| 86 | 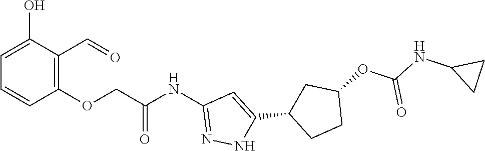 [SMILES: O=Cc1c(O)cccc1OCC(=O)Nc1cc([C@H]2CC[C@@H](OC(=O)NC3CC3)C2)[nH]n1] |
| 87 | 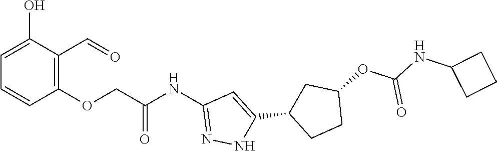 [SMILES: O=Cc1c(O)cccc1OCC(=O)Nc1cc([C@H]2CC[C@@H](OC(=O)NC3CCC3)C2)[nH]n1] |
| 88 | 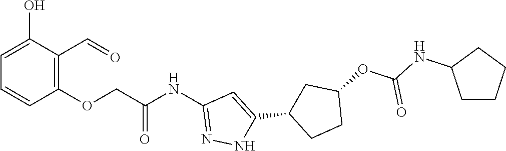 [SMILES: O=Cc1c(O)cccc1OCC(=O)Nc1cc([C@H]2CC[C@@H](OC(=O)NC3CCCC3)C2)[nH]n1] |
| 89 | 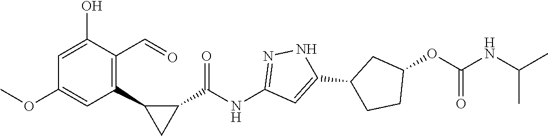 [SMILES: COc1cc(O)c(C=O)c([C@@H]2C[C@H]2C(=O)Nc2cc([C@H]3CC[C@@H](OC(=O)NC(C)C)C3)[nH]n2)c1] |
| 90 | 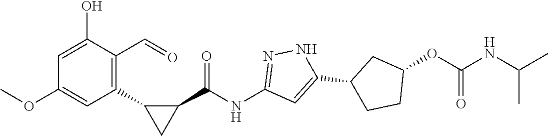 [SMILES: COc1cc(O)c(C=O)c([C@H]2C[C@@H]2C(=O)Nc2cc([C@H]3CC[C@@H](OC(=O)NC(C)C)C3)[nH]n2)c1] |
| 91 | 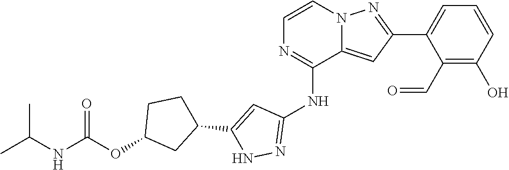 [SMILES: CC(C)NC(=O)O[C@@H]1CC[C@H](c2cc(Nc3nccn4nc(-c5cccc(O)c5C=O)cc34)n[nH]2)C1] |
| 92 | 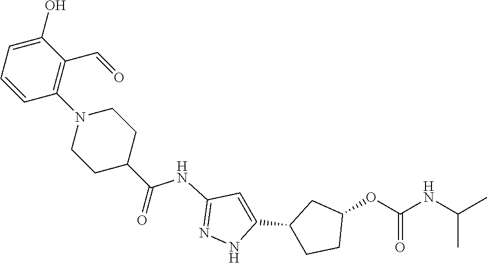 [SMILES: CC(C)NC(=O)O[C@@H]1CC[C@H](c2cc(NC(=O)C3CCN(c4cccc(O)c4C=O)CC3)n[nH]2)C1] |
| 93 | 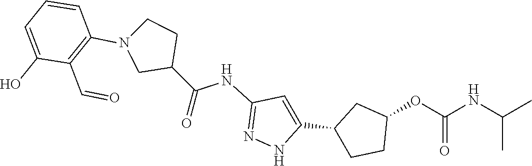 [SMILES: CC(C)NC(=O)O[C@@H]1CC[C@H](c2cc(NC(=O)C3CCN(c4cccc(O)c4C=O)C3)n[nH]2)C1] |
| 94 | 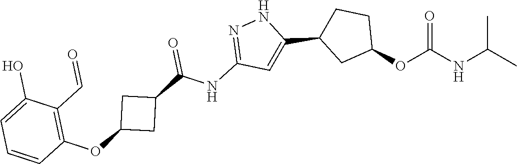 [SMILES: CC(C)NC(=O)O[C@@H]1CC[C@H](c2cc(NC(=O)[C@H]3C[C@@H](Oc4cccc(O)c4C=O)C3)n[nH]2)C1] |
| 95 | 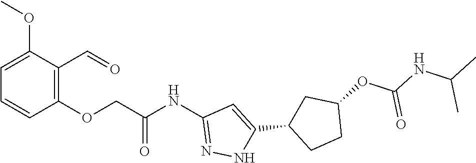 [SMILES: COc1cccc(OCC(=O)Nc2cc([C@H]3CC[C@@H](OC(=O)NC(C)C)C3)[nH]n2)c1C=O] |
| 96 | 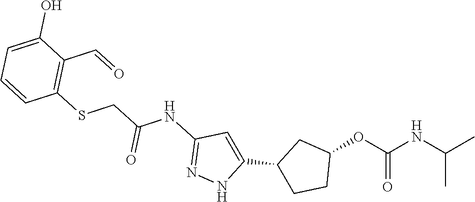 [SMILES: CC(C)NC(=O)O[C@@H]1CC[C@H](c2cc(NC(=O)CSc3cccc(O)c3C=O)n[nH]2)C1] |
| 97 | 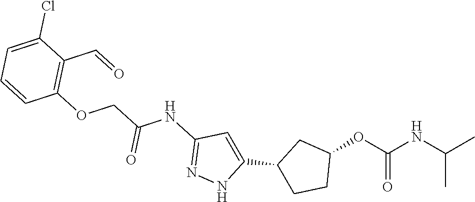 [SMILES: CC(C)NC(=O)O[C@@H]1CC[C@H](c2cc(NC(=O)COc3cccc(Cl)c3C=O)n[nH]2)C1] |
| 98 | 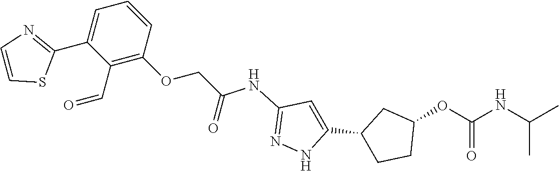 [SMILES: CC(C)NC(=O)O[C@@H]1CC[C@H](c2cc(NC(=O)COc3cccc(-c4nccs4)c3C=O)n[nH]2)C1] |
| 99 | 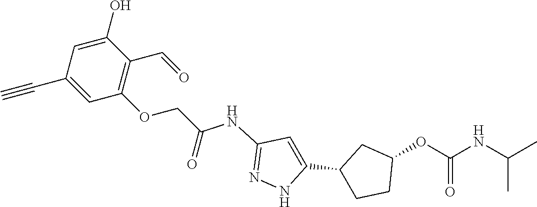 [SMILES: C#Cc1cc(O)c(C=O)c(OCC(=O)Nc2cc([C@H]3CC[C@@H](OC(=O)NC(C)C)C3)[nH]n2)c1] |
| 100 | 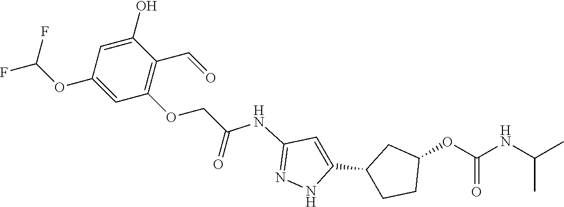 [SMILES: CC(C)NC(=O)O[C@@H]1CC[C@H](c2cc(NC(=O)COc3cc(OC(F)F)cc(O)c3C=O)n[nH]2)C1] |
| 101 | 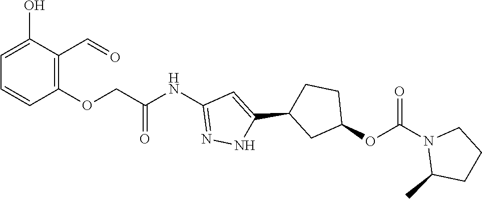 [SMILES: C[C@@H]1CCCN1C(=O)O[C@@H]1CC[C@H](c2cc(NC(=O)COc3cccc(O)c3C=O)n[nH]2)C1] |
| 102 | 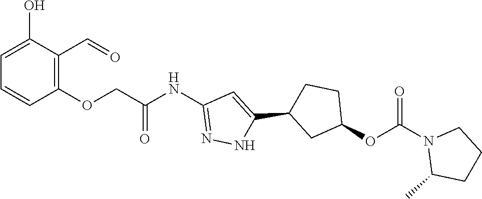 [SMILES: C[C@H]1CCCN1C(=O)O[C@@H]1CC[C@H](c2cc(NC(=O)COc3cccc(O)c3C=O)n[nH]2)C1] |
| 103 | 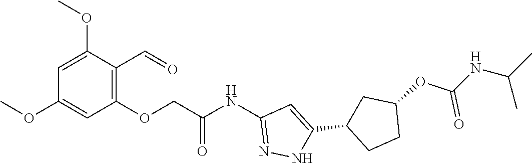 [SMILES: COc1cc(OC)c(C=O)c(OCC(=O)Nc2cc([C@H]3CC[C@@H](OC(=O)NC(C)C)C3)[nH]n2)c1] |
| 105 | 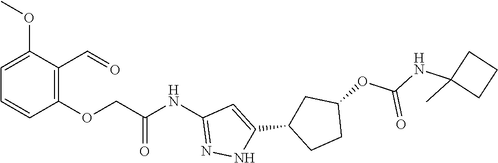 [SMILES: COc1cccc(OCC(=O)Nc2cc([C@H]3CC[C@@H](OC(=O)NC4(C)CCC4)C3)[nH]n2)c1C=O] |
| 106 | 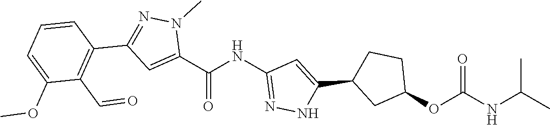 [SMILES: COc1cccc(-c2cc(C(=O)Nc3cc([C@H]4CC[C@@H](OC(=O)NC(C)C)C4)[nH]n3)n(C)n2)c1C=O] |
| 107 | 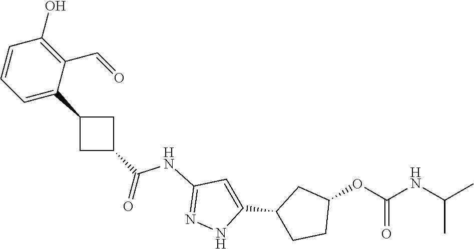 [SMILES: CC(C)NC(=O)O[C@@H]1CC[C@H](c2cc(NC(=O)[C@H]3C[C@H](c4cccc(O)c4C=O)C3)n[nH]2)C1] |
| 108 | 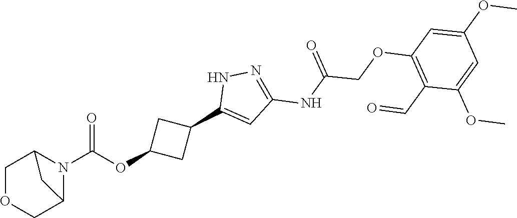 [SMILES: COc1cc(OC)c(C=O)c(OCC(=O)Nc2cc([C@H]3C[C@@H](OC(=O)N4C5COCC4C5)C3)[nH]n2)c1] |
| 109 | 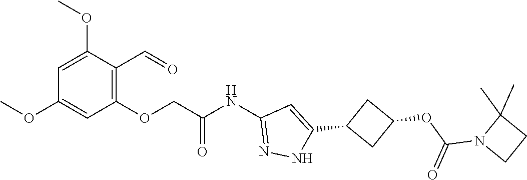 [SMILES: COc1cc(OC)c(C=O)c(OCC(=O)Nc2cc([C@H]3C[C@@H](OC(=O)N4CCC4(C)C)C3)[nH]n2)c1] |
| 110 | 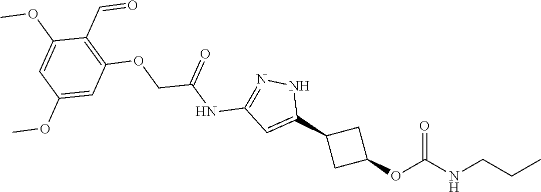 [SMILES: CCCNC(=O)O[C@H]1C[C@@H](c2cc(NC(=O)COc3cc(OC)cc(OC)c3C=O)n[nH]2)C1] |
| 111 | 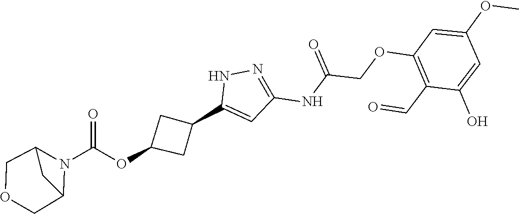 [SMILES: COc1cc(O)c(C=O)c(OCC(=O)Nc2cc([C@H]3C[C@@H](OC(=O)N4C5COCC4C5)C3)[nH]n2)c1] |
| 112 | 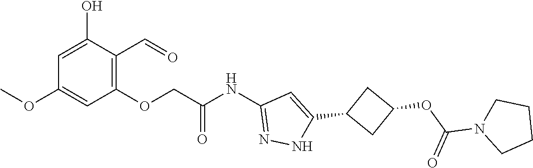 [SMILES: COc1cc(O)c(C=O)c(OCC(=O)Nc2cc([C@H]3C[C@@H](OC(=O)N4CCCC4)C3)[nH]n2)c1] |
| 113 | 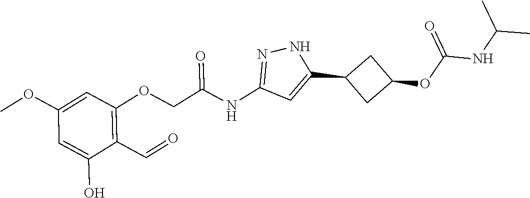 [SMILES: COc1cc(O)c(C=O)c(OCC(=O)Nc2cc([C@H]3C[C@@H](OC(=O)NC(C)C)C3)[nH]n2)c1] |
| 114 | 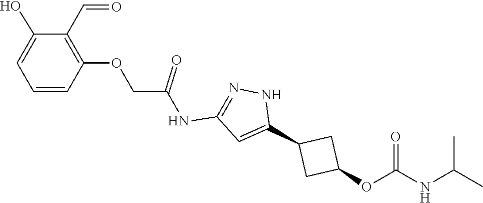 [SMILES: CC(C)NC(=O)O[C@H]1C[C@@H](c2cc(NC(=O)COc3cccc(O)c3C=O)n[nH]2)C1] |
| 115 | 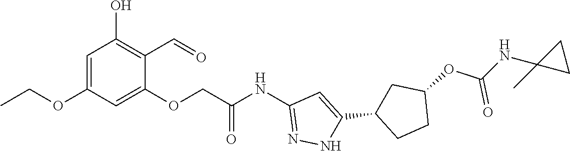 [SMILES: CCOc1cc(O)c(C=O)c(OCC(=O)Nc2cc([C@H]3CC[C@@H](OC(=O)NC4(C)CC4)C3)[nH]n2)c1] |
| 116 | 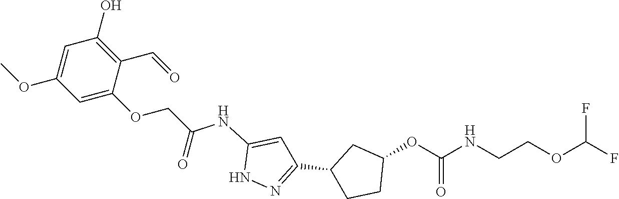 [SMILES: COc1cc(O)c(C=O)c(OCC(=O)Nc2cc([C@H]3CC[C@@H](OC(=O)NCCOC(F)F)C3)n[nH]2)c1] |
| 117 | 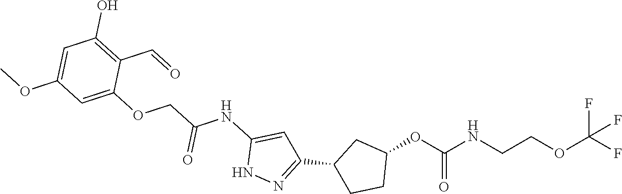 [SMILES: COc1cc(O)c(C=O)c(OCC(=O)Nc2cc([C@H]3CC[C@@H](OC(=O)NCCOC(F)(F)F)C3)n[nH]2)c1] |
| 118 | 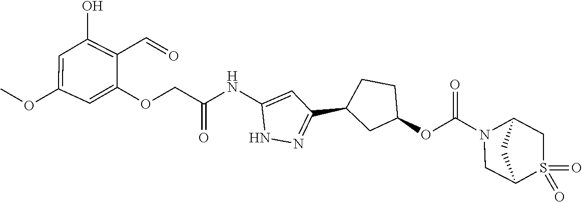 [SMILES: COc1cc(O)c(C=O)c(OCC(=O)Nc2cc([C@H]3CC[C@@H](OC(=O)N4C[C@H]5C[C@@H]4CS5(=O)=O)C3)n[nH]2)c1] |
| 119 | 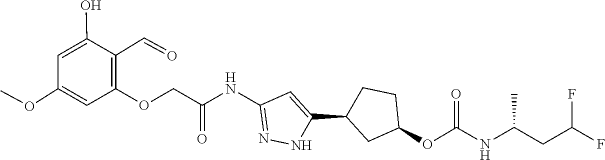 [SMILES: COc1cc(O)c(C=O)c(OCC(=O)Nc2cc([C@H]3CC[C@@H](OC(=O)N[C@H](C)CC(F)F)C3)[nH]n2)c1] |
| 120 | 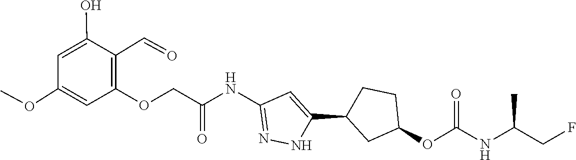 [SMILES: COc1cc(O)c(C=O)c(OCC(=O)Nc2cc([C@H]3CC[C@@H](OC(=O)N[C@@H](C)CF)C3)[nH]n2)c1] |
| 121 | 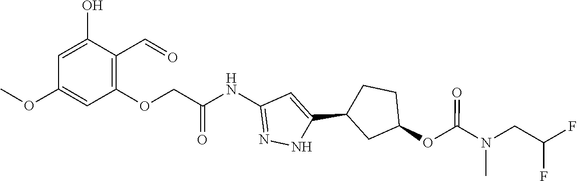 [SMILES: COc1cc(O)c(C=O)c(OCC(=O)Nc2cc([C@H]3CC[C@@H](OC(=O)N(C)CC(F)F)C3)[nH]n2)c1] |
| 122 | 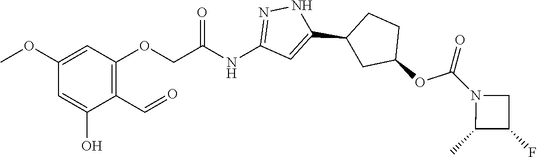 [SMILES: COc1cc(O)c(C=O)c(OCC(=O)Nc2cc([C@H]3CC[C@@H](OC(=O)N4C[C@H](F)[C@@H]4C)C3)[nH]n2)c1] |
| 123 | 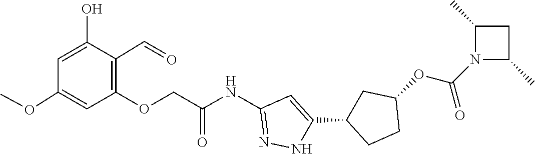 |
| 124 | 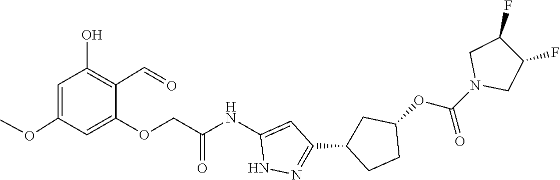 [SMILES: COc1cc(O)c(C=O)c(OCC(=O)Nc2cc([C@H]3CC[C@@H](OC(=O)N4C[C@@H](F)[C@H](F)C4)C3)n[nH]2)c1] |
| 125 | 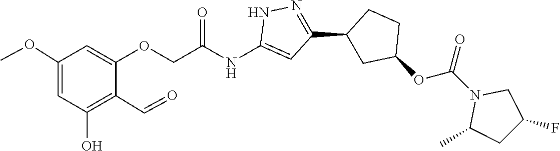 [SMILES: COc1cc(O)c(C=O)c(OCC(=O)Nc2cc([C@H]3CC[C@@H](OC(=O)N4C[C@H](F)C[C@@H]4C)C3)n[nH]2)c1] |
| 126 | 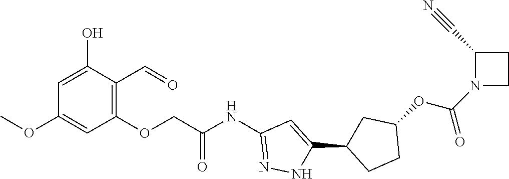 [SMILES: COc1cc(O)c(C=O)c(OCC(=O)Nc2cc([C@@H]3CC[C@@H](OC(=O)N4CC[C@H]4C#N)C3)[nH]n2)c1] |
| 127 | 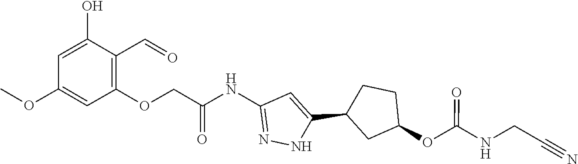 [SMILES: COc1cc(O)c(C=O)c(OCC(=O)Nc2cc([C@H]3CC[C@@H](OC(=O)NCC#N)C3)[nH]n2)c1] |
| 128 | 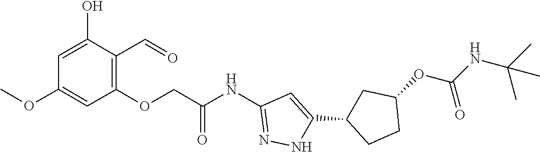 [SMILES: COc1cc(O)c(C=O)c(OCC(=O)Nc2cc([C@H]3CC[C@@H](OC(=O)NC(C)(C)C)C3)[nH]n2)c1] |
| 129 | 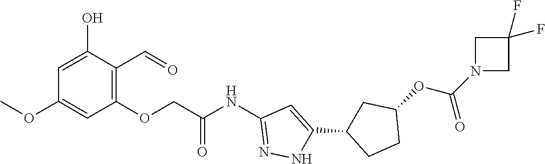 [SMILES: COc1cc(O)c(C=O)c(OCC(=O)Nc2cc([C@H]3CC[C@@H](OC(=O)N4CC(F)(F)C4)C3)[nH]n2)c1] |
| 130 | 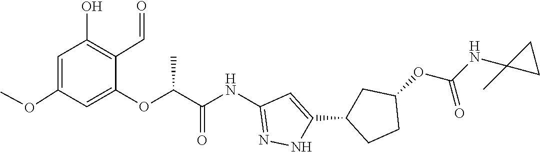 [SMILES: COc1cc(O)c(C=O)c(O[C@H](C)C(=O)Nc2cc([C@H]3CC[C@@H](OC(=O)NC4(C)CC4)C3)[nH]n2)c1] |
| 131 | 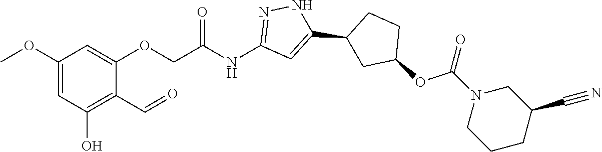 [SMILES: COc1cc(O)c(C=O)c(OCC(=O)Nc2cc([C@H]3CC[C@@H](OC(=O)N4CCC[C@H](C#N)C4)C3)[nH]n2)c1] |
| 132 | 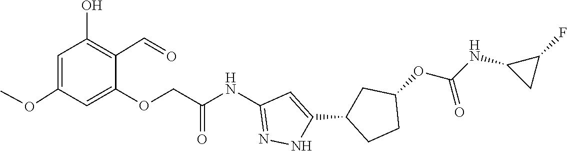 [SMILES: COc1cc(O)c(C=O)c(OCC(=O)Nc2cc([C@H]3CC[C@@H](OC(=O)N[C@H]4C[C@H]4F)C3)[nH]n2)c1] |
| 133 | 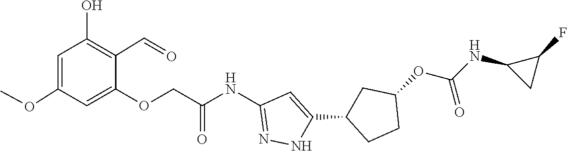 [SMILES: COc1cc(O)c(C=O)c(OCC(=O)Nc2cc([C@H]3CC[C@@H](OC(=O)N[C@@H]4C[C@@H]4F)C3)[nH]n2)c1] |
| 134 | 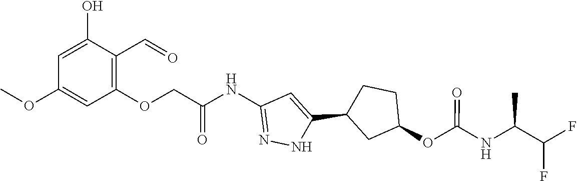 [SMILES: COc1cc(O)c(C=O)c(OCC(=O)Nc2cc([C@H]3CC[C@@H](OC(=O)N[C@@H](C)C(F)F)C3)[nH]n2)c1] |
| 135 | 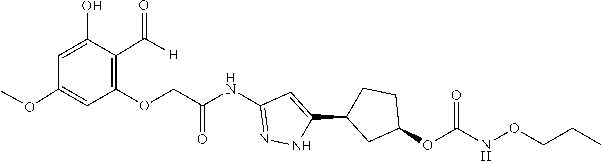 [SMILES: CCCONC(=O)O[C@@H]1CC[C@H](c2cc(NC(=O)COc3cc(OC)cc(O)c3C=O)n[nH]2)C1] |
| 136 | 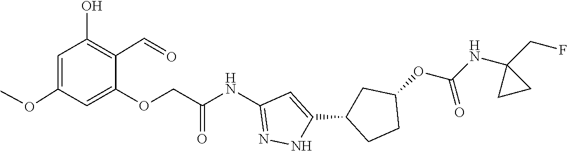 [SMILES: COc1cc(O)c(C=O)c(OCC(=O)Nc2cc([C@H]3CC[C@@H](OC(=O)NC4(CF)CC4)C3)[nH]n2)c1] |
| 137 | 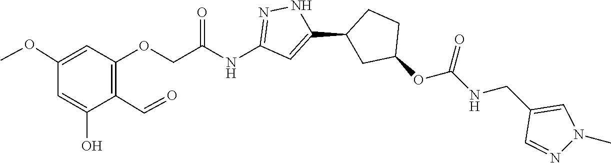 |
| 138 | 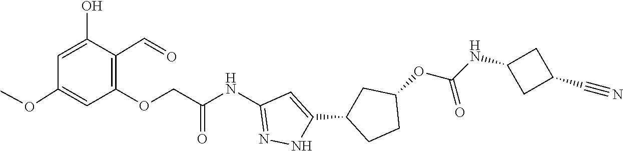 [SMILES: COc1cc(O)c(C=O)c(OCC(=O)Nc2cc([C@H]3CC[C@@H](OC(=O)N[C@H]4C[C@@H](C#N)C4)C3)[nH]n2)c1] |
| 139 | 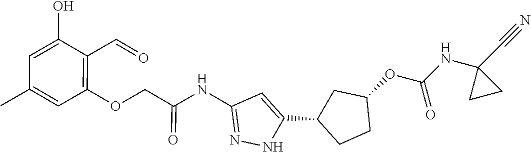 [SMILES: Cc1cc(O)c(C=O)c(OCC(=O)Nc2cc([C@H]3CC[C@@H](OC(=O)NC4(C#N)CC4)C3)[nH]n2)c1] |
| 140 | 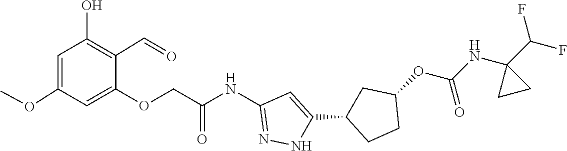 [SMILES: COc1cc(O)c(C=O)c(OCC(=O)Nc2cc([C@H]3CC[C@@H](OC(=O)NC4(C(F)F)CC4)C3)[nH]n2)c1] |
| 141 | 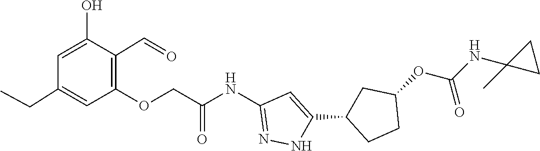 [SMILES: CCc1cc(O)c(C=O)c(OCC(=O)Nc2cc([C@H]3CC[C@@H](OC(=O)NC4(C)CC4)C3)[nH]n2)c1] |
| 142 | 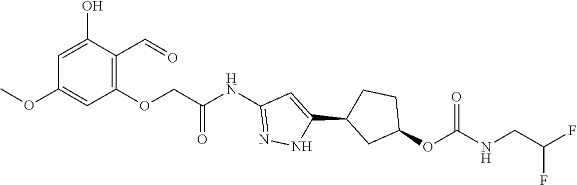 [SMILES: COc1cc(O)c(C=O)c(OCC(=O)Nc2cc([C@H]3CC[C@@H](OC(=O)NCC(F)F)C3)[nH]n2)c1] |
| 143 | 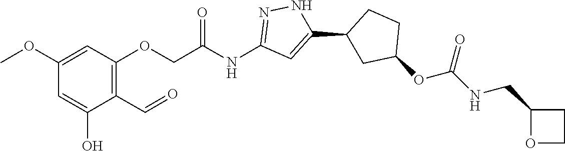 [SMILES: COc1cc(O)c(C=O)c(OCC(=O)Nc2cc([C@H]3CC[C@@H](OC(=O)NC[C@H]4CCO4)C3)[nH]n2)c1] |
| 144 | 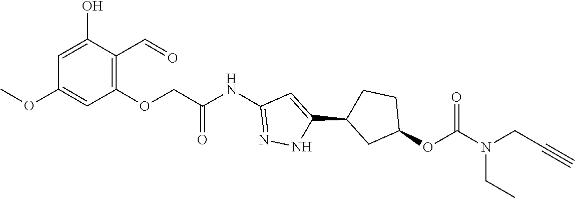 [SMILES: C#CCN(CC)C(=O)O[C@@H]1CC[C@H](c2cc(NC(=O)COc3cc(OC)cc(O)c3C=O)n[nH]2)C1] |
| 145 | 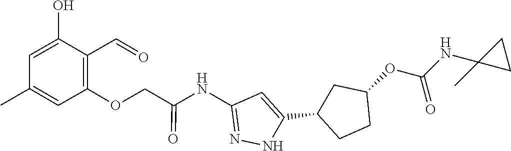 [SMILES: Cc1cc(O)c(C=O)c(OCC(=O)Nc2cc([C@H]3CC[C@@H](OC(=O)NC4(C)CC4)C3)[nH]n2)c1] |
| 146 | 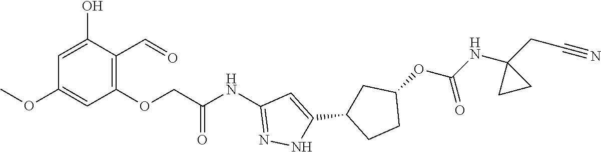 [SMILES: COc1cc(O)c(C=O)c(OCC(=O)Nc2cc([C@H]3CC[C@@H](OC(=O)NC4(CC#N)CC4)C3)[nH]n2)c1] |
| 147 | 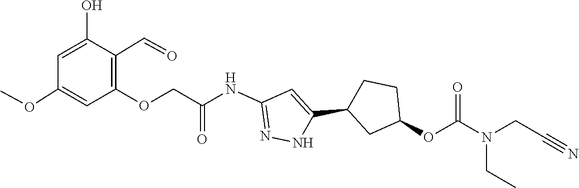 [SMILES: CCN(CC#N)C(=O)O[C@@H]1CC[C@H](c2cc(NC(=O)COc3cc(OC)cc(O)c3C=O)n[nH]2)C1] |
| 148 | 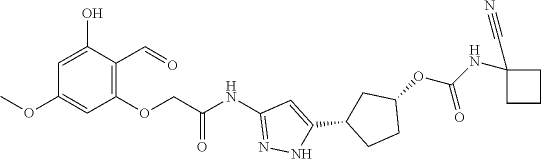 [SMILES: COc1cc(O)c(C=O)c(OCC(=O)Nc2cc([C@H]3CC[C@@H](OC(=O)NC4(C#N)CCC4)C3)[nH]n2)c1] |
| 149 | 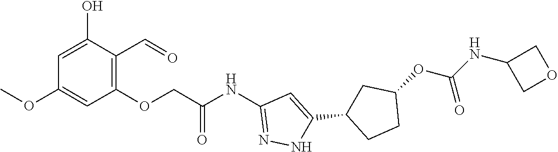 [SMILES: COc1cc(O)c(C=O)c(OCC(=O)Nc2cc([C@H]3CC[C@@H](OC(=O)NC4COC4)C3)[nH]n2)c1] |
| 150 | 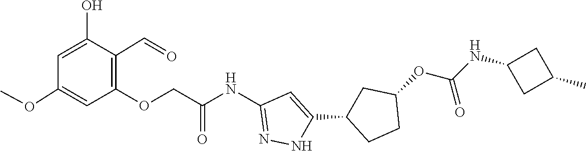 |
| 151 | 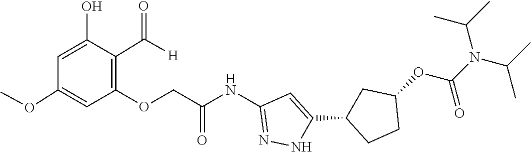 [SMILES: COc1cc(O)c(C=O)c(OCC(=O)Nc2cc([C@H]3CC[C@@H](OC(=O)N(C(C)C)C(C)C)C3)[nH]n2)c1] |
| 152 | 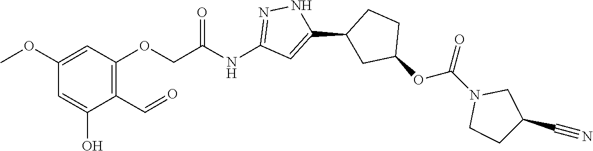 [SMILES: COc1cc(O)c(C=O)c(OCC(=O)Nc2cc([C@H]3CC[C@@H](OC(=O)N4CC[C@H](C#N)C4)C3)[nH]n2)c1] |
| 153 | 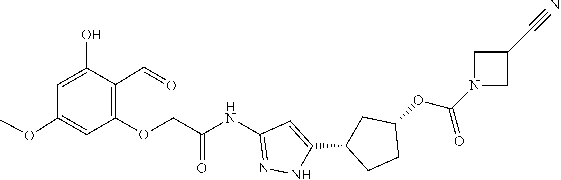 [SMILES: COc1cc(O)c(C=O)c(OCC(=O)Nc2cc([C@H]3CC[C@@H](OC(=O)N4CC(C#N)C4)C3)[nH]n2)c1] |
| 154 | 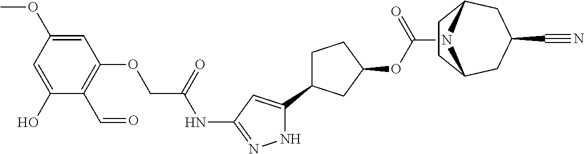 [SMILES: COc1cc(O)c(C=O)c(OCC(=O)Nc2cc([C@H]3CC[C@@H](OC(=O)N4[C@@H]5CC[C@H]4C[C@H](C#N)C5)C3)[nH]n2)c1] |
| 155 | 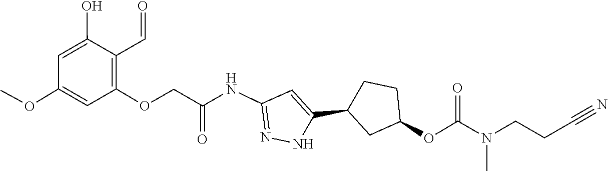 [SMILES: COc1cc(O)c(C=O)c(OCC(=O)Nc2cc([C@H]3CC[C@@H](OC(=O)N(C)CCC#N)C3)[nH]n2)c1] |
| 156 | 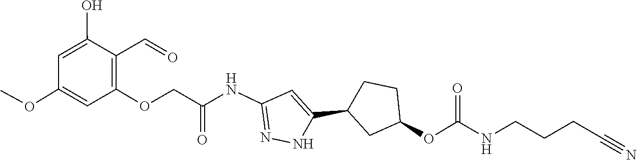 [SMILES: COc1cc(O)c(C=O)c(OCC(=O)Nc2cc([C@H]3CC[C@@H](OC(=O)NCCCC#N)C3)[nH]n2)c1] |
| 157 | 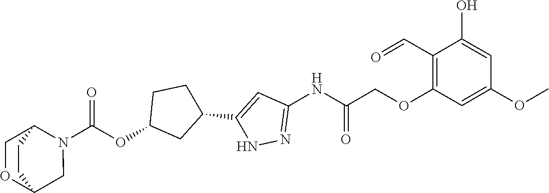 [SMILES: COc1cc(O)c(C=O)c(OCC(=O)Nc2cc([C@H]3CC[C@@H](OC(=O)N4C[C@@H]5CC[C@H]4CO5)C3)[nH]n2)c1] |
| 158 | 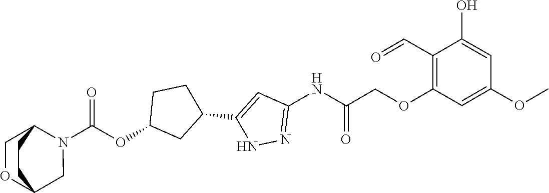 [SMILES: COc1cc(O)c(C=O)c(OCC(=O)Nc2cc([C@H]3CC[C@@H](OC(=O)N4C[C@H]5CC[C@@H]4CO5)C3)[nH]n2)c1] |
| 159 | 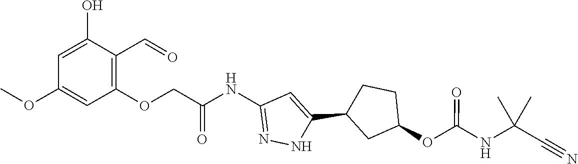 [SMILES: COc1cc(O)c(C=O)c(OCC(=O)Nc2cc([C@H]3CC[C@@H](OC(=O)NC(C)(C)C#N)C3)[nH]n2)c1] |
| 160 | 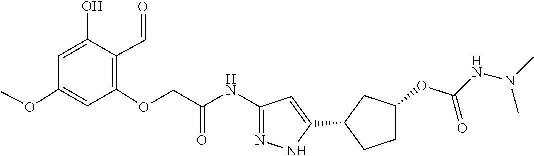 |
| 161 | 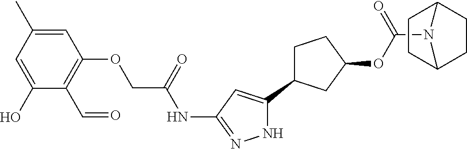 [SMILES: Cc1cc(O)c(C=O)c(OCC(=O)Nc2cc([C@H]3CC[C@@H](OC(=O)N4C5CCC4CC5)C3)[nH]n2)c1] |
| 162 | 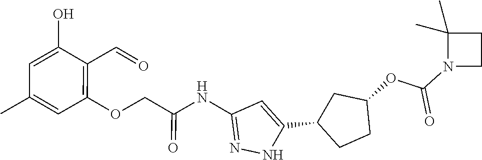 [SMILES: Cc1cc(O)c(C=O)c(OCC(=O)Nc2cc([C@H]3CC[C@@H](OC(=O)N4CCC4(C)C)C3)[nH]n2)c1] |
| 163 | 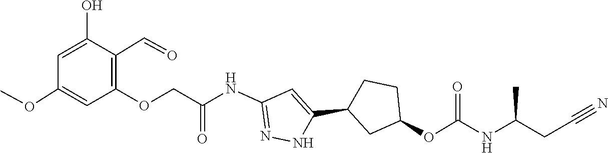 [SMILES: COc1cc(O)c(C=O)c(OCC(=O)Nc2cc([C@H]3CC[C@@H](OC(=O)N[C@@H](C)CC#N)C3)[nH]n2)c1] |
| 164 | 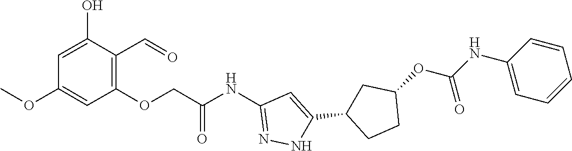 [SMILES: COc1cc(O)c(C=O)c(OCC(=O)Nc2cc([C@H]3CC[C@@H](OC(=O)Nc4ccccc4)C3)[nH]n2)c1] |
| 165 | 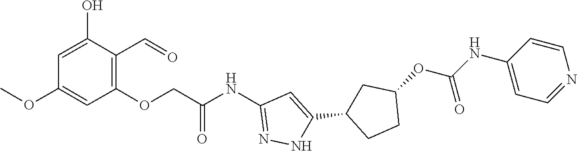 [SMILES: COc1cc(O)c(C=O)c(OCC(=O)Nc2cc([C@H]3CC[C@@H](OC(=O)Nc4ccncc4)C3)[nH]n2)c1] |
| 166 | 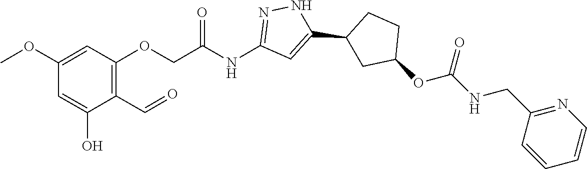 [SMILES: COc1cc(O)c(C=O)c(OCC(=O)Nc2cc([C@H]3CC[C@@H](OC(=O)NCc4ccccn4)C3)[nH]n2)c1] |
| 167 | 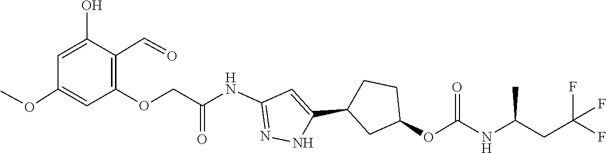 [SMILES: COc1cc(O)c(C=O)c(OCC(=O)Nc2cc([C@H]3CC[C@@H](OC(=O)N[C@@H](C)CC(F)(F)F)C3)[nH]n2)c1] |
| 168 | 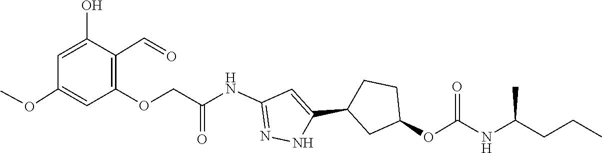 [SMILES: CCC[C@H](C)NC(=O)O[C@@H]1CC[C@H](c2cc(NC(=O)COc3cc(OC)cc(O)c3C=O)n[nH]2)C1] |
| 169 | 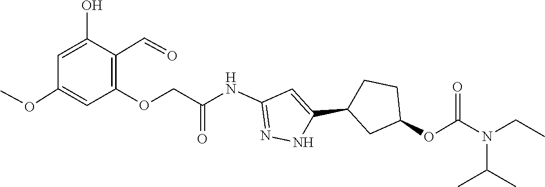 [SMILES: CCN(C(=O)O[C@@H]1CC[C@H](c2cc(NC(=O)COc3cc(OC)cc(O)c3C=O)n[nH]2)C1)C(C)C] |
| 170 | 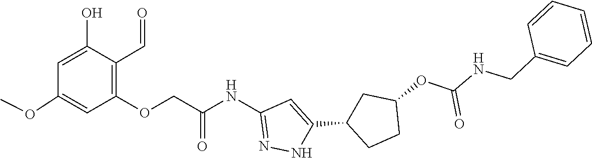 [SMILES: COc1cc(O)c(C=O)c(OCC(=O)Nc2cc([C@H]3CC[C@@H](OC(=O)NCc4ccccc4)C3)[nH]n2)c1] |
| 171 | 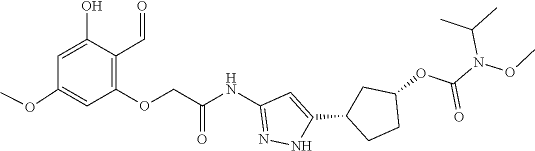 |
| 172 | 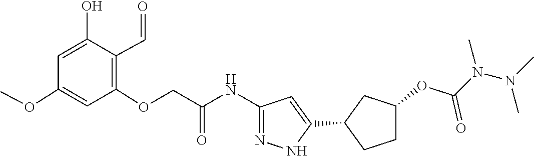 |
| 173 | 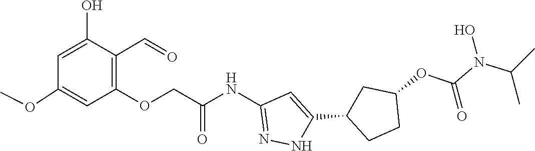 [SMILES: COc1cc(O)c(C=O)c(OCC(=O)Nc2cc([C@H]3CC[C@@H](OC(=O)N(O)C(C)C)C3)[nH]n2)c1] |
| 174 | 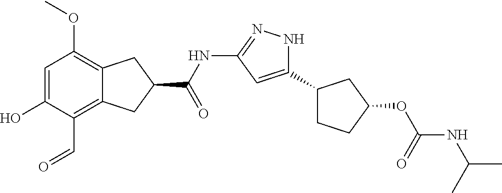 [SMILES: COc1cc(O)c(C=O)c2c1C[C@@H](C(=O)Nc1cc([C@H]3CC[C@@H](OC(=O)NC(C)C)C3)[nH]n1)C2] |
| 175 | 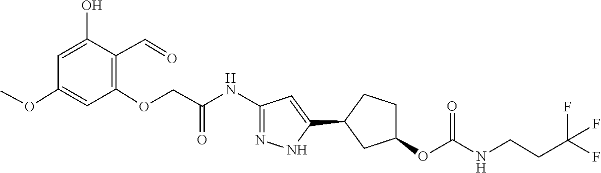 [SMILES: COc1cc(O)c(C=O)c(OCC(=O)Nc2cc([C@H]3CC[C@@H](OC(=O)NCCC(F)(F)F)C3)[nH]n2)c1] |
| 176 | 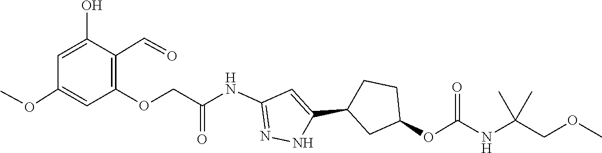 |
| 177 | 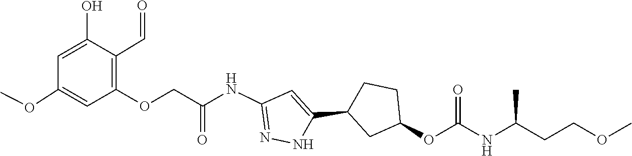 |
| 178 | 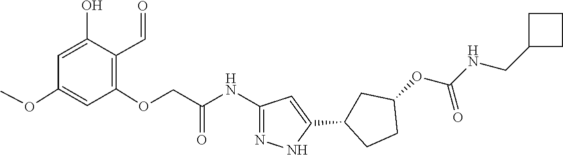 [SMILES: COc1cc(O)c(C=O)c(OCC(=O)Nc2cc([C@H]3CC[C@@H](OC(=O)NCC4CCC4)C3)[nH]n2)c1] |
| 179 | 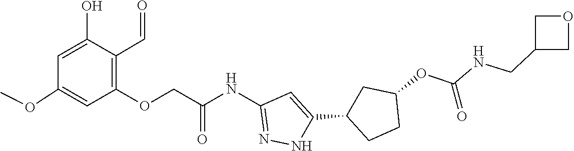 [SMILES: COc1cc(O)c(C=O)c(OCC(=O)Nc2cc([C@H]3CC[C@@H](OC(=O)NCC4COC4)C3)[nH]n2)c1] |
| 180 | 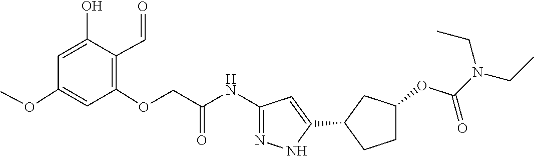 [SMILES: CCN(CC)C(=O)O[C@@H]1CC[C@H](c2cc(NC(=O)COc3cc(OC)cc(O)c3C=O)n[nH]2)C1] |
| 181 | 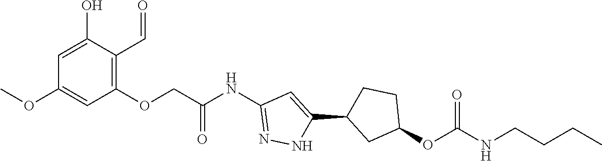 [SMILES: CCCCNC(=O)O[C@@H]1CC[C@H](c2cc(NC(=O)COc3cc(OC)cc(O)c3C=O)n[nH]2)C1] |
| 182 | 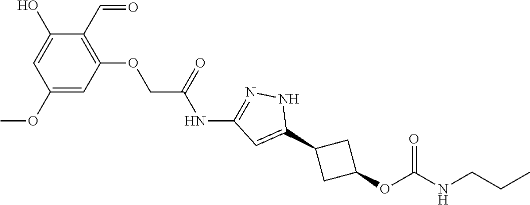 [SMILES: CCCNC(=O)O[C@H]1C[C@@H](c2cc(NC(=O)COc3cc(OC)cc(O)c3C=O)n[nH]2)C1] |
| 183 | 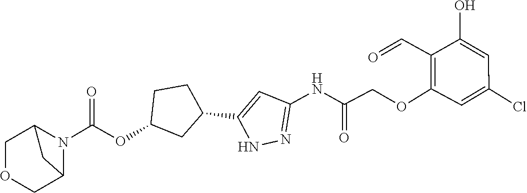 [SMILES: O=Cc1c(O)cc(Cl)cc1OCC(=O)Nc1cc([C@H]2CC[C@@H](OC(=O)N3C4COCC3C4)C2)[nH]n1] |
| 184 | 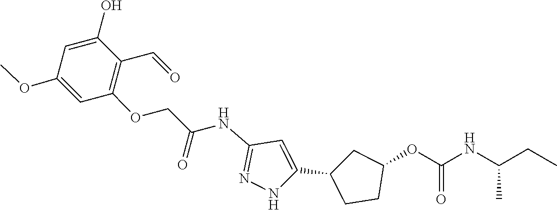 [SMILES: CC[C@H](C)NC(=O)O[C@@H]1CC[C@H](c2cc(NC(=O)COc3cc(OC)cc(O)c3C=O)n[nH]2)C1] |
| 185 | 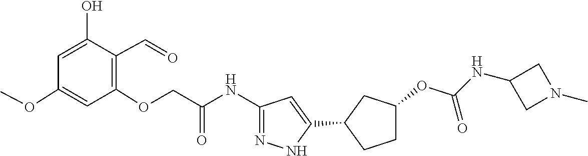 |
| 186 | 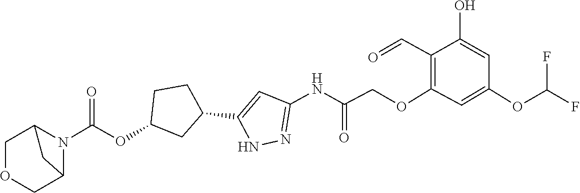 [SMILES: O=Cc1c(O)cc(OC(F)F)cc1OCC(=O)Nc1cc([C@H]2CC[C@@H](OC(=O)N3C4COCC3C4)C2)[nH]n1] |
| 187 | 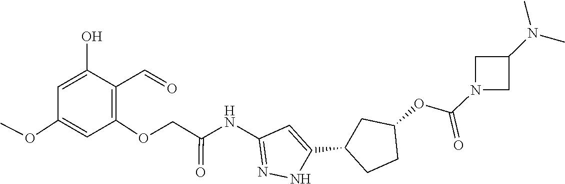 |
| 188 | 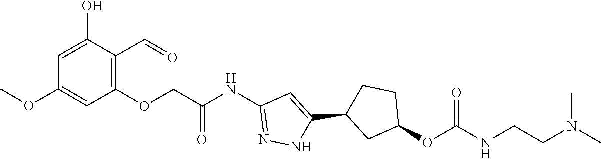 |
| 189 | 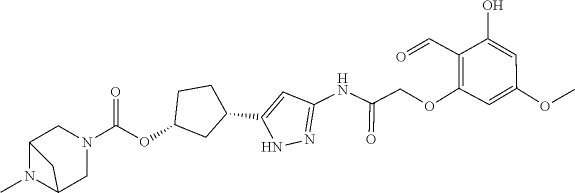 |
| 190 | 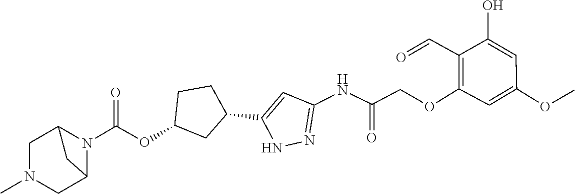 |
| 191 | 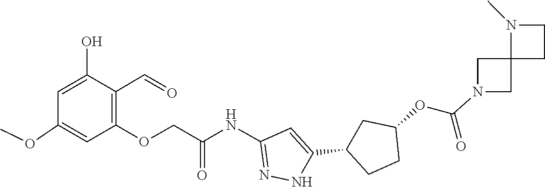 [SMILES: COc1cc(O)c(C=O)c(OCC(=O)Nc2cc([C@H]3CC[C@@H](OC(=O)N4CC5(CCN5C)C4)C3)[nH]n2)c1] |
| 192 | 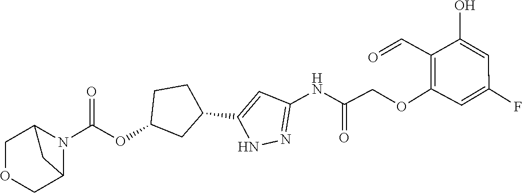 [SMILES: O=Cc1c(O)cc(F)cc1OCC(=O)Nc1cc([C@H]2CC[C@@H](OC(=O)N3C4COCC3C4)C2)[nH]n1] |
| 193 | 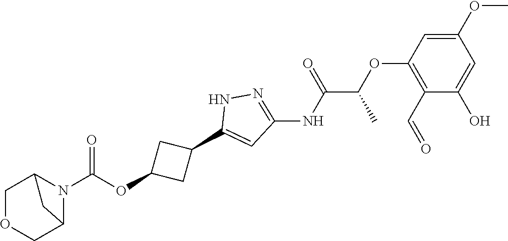 [SMILES: COc1cc(O)c(C=O)c(O[C@H](C)C(=O)Nc2cc([C@H]3C[C@@H](OC(=O)N4C5COCC4C5)C3)[nH]n2)c1] |
| 194 | 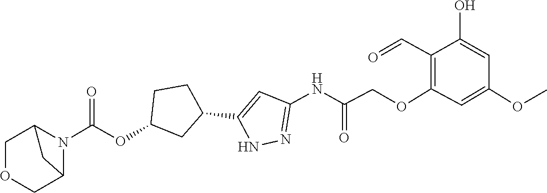 [SMILES: COc1cc(O)c(C=O)c(OCC(=O)Nc2cc([C@H]3CC[C@@H](OC(=O)N4C5COCC4C5)C3)[nH]n2)c1] |
| 195 | 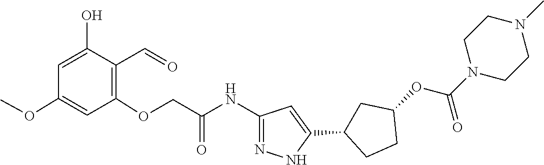 |
| 196 | 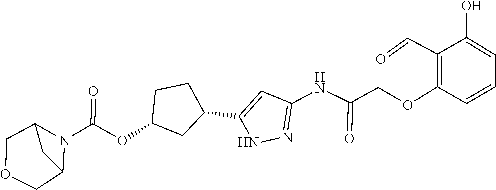 [SMILES: O=Cc1c(O)cccc1OCC(=O)Nc1cc([C@H]2CC[C@@H](OC(=O)N3C4COCC3C4)C2)[nH]n1] |
| 197 | 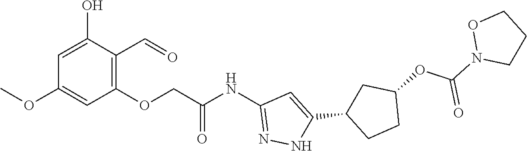 [SMILES: COc1cc(O)c(C=O)c(OCC(=O)Nc2cc([C@H]3CC[C@@H](OC(=O)N4CCCO4)C3)[nH]n2)c1] |
| 198 | 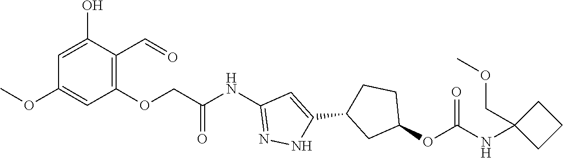 [SMILES: COCC1(NC(=O)O[C@@H]2CC[C@@H](c3cc(NC(=O)COc4cc(OC)cc(O)c4C=O)n[nH]3)C2)CCC1] |
| 199 | 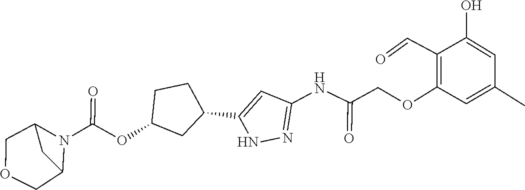 [SMILES: Cc1cc(O)c(C=O)c(OCC(=O)Nc2cc([C@H]3CC[C@@H](OC(=O)N4C5COCC4C5)C3)[nH]n2)c1] |
| 200 | 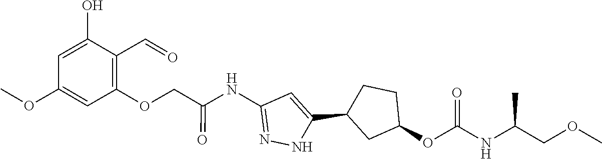 |
| 201 | 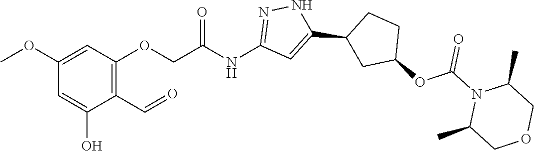 [SMILES: COc1cc(O)c(C=O)c(OCC(=O)Nc2cc([C@H]3CC[C@@H](OC(=O)N4[C@H](C)COC[C@@H]4C)C3)[nH]n2)c1] |
| 202 | 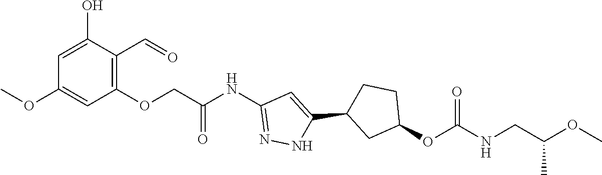 |
| 203 | 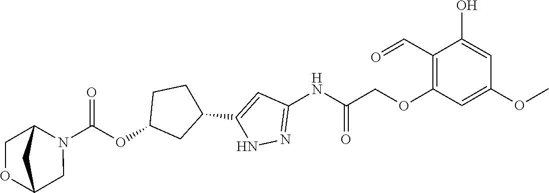 [SMILES: COc1cc(O)c(C=O)c(OCC(=O)Nc2cc([C@H]3CC[C@@H](OC(=O)N4C[C@H]5C[C@@H]4CO5)C3)[nH]n2)c1] |
| 204 | 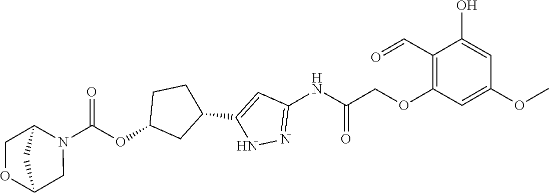 [SMILES: COc1cc(O)c(C=O)c(OCC(=O)Nc2cc([C@H]3CC[C@@H](OC(=O)N4C[C@@H]5C[C@H]4CO5)C3)[nH]n2)c1] |
| 205 | 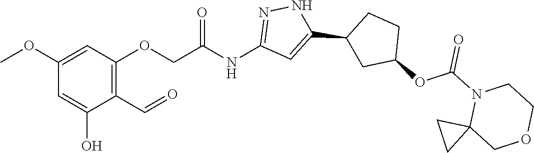 [SMILES: COc1cc(O)c(C=O)c(OCC(=O)Nc2cc([C@H]3CC[C@@H](OC(=O)N4CCOCC45CC5)C3)[nH]n2)c1] |
| 206 | 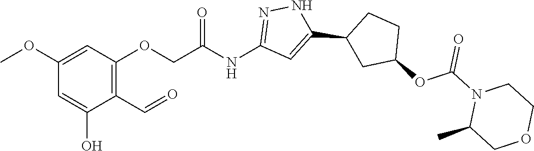 [SMILES: COc1cc(O)c(C=O)c(OCC(=O)Nc2cc([C@H]3CC[C@@H](OC(=O)N4CCOC[C@H]4C)C3)[nH]n2)c1] |
| 207 | 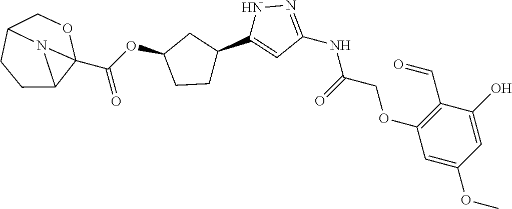 [SMILES: COc1cc(O)c(C=O)c(OCC(=O)Nc2cc([C@H]3CC[C@@H](OC(=O)C45OCC6CCC4N65)C3)[nH]n2)c1] |
| 208 | 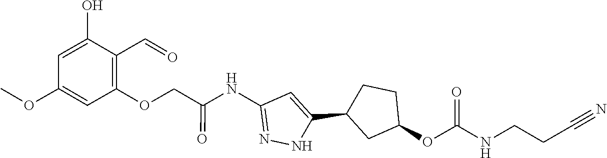 [SMILES: COc1cc(O)c(C=O)c(OCC(=O)Nc2cc([C@H]3CC[C@@H](OC(=O)NCCC#N)C3)[nH]n2)c1] |
| 209 | 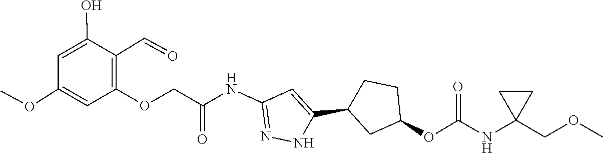 |
| 210 | 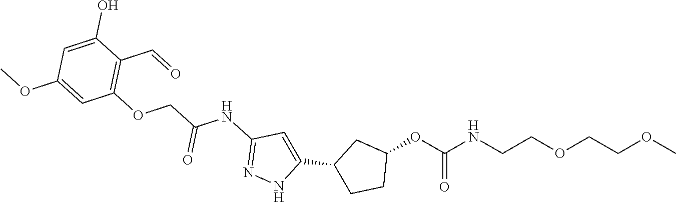 |
| 211 | 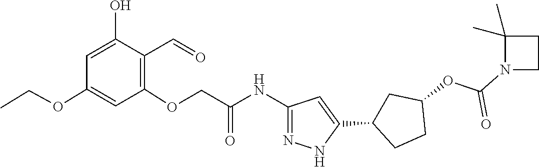 [SMILES: CCOc1cc(O)c(C=O)c(OCC(=O)Nc2cc([C@H]3CC[C@@H](OC(=O)N4CCC4(C)C)C3)[nH]n2)c1] |
| 212 | 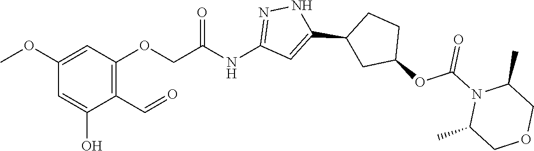 [SMILES: COc1cc(O)c(C=O)c(OCC(=O)Nc2cc([C@H]3CC[C@@H](OC(=O)N4[C@@H](C)COC[C@@H]4C)C3)[nH]n2)c1] |
| 213 | 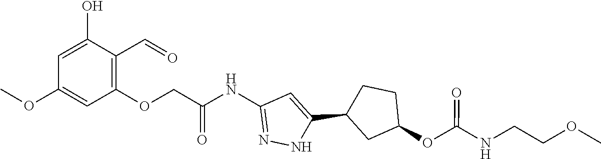 |
| 214 | 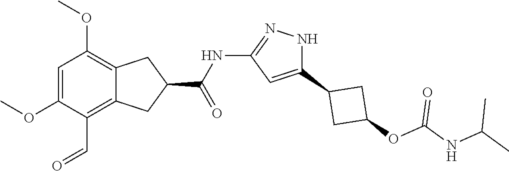 [SMILES: COc1cc(OC)c2c(c1C=O)C[C@H](C(=O)Nc1cc([C@H]3C[C@@H](OC(=O)NC(C)C)C3)[nH]n1)C2] |
| 215 | 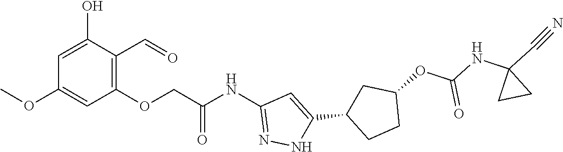 [SMILES: COc1cc(O)c(C=O)c(OCC(=O)Nc2cc([C@H]3CC[C@@H](OC(=O)NC4(C#N)CC4)C3)[nH]n2)c1] |
| 216 | 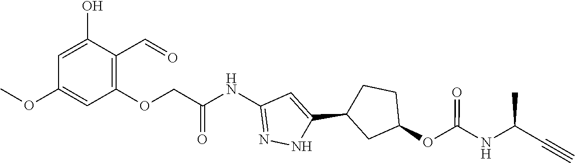 [SMILES: C#C[C@H](C)NC(=O)O[C@@H]1CC[C@H](c2cc(NC(=O)COc3cc(OC)cc(O)c3C=O)n[nH]2)C1] |
| 217 | 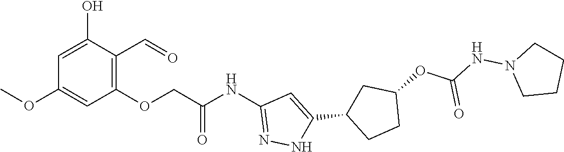 [SMILES: COc1cc(O)c(C=O)c(OCC(=O)Nc2cc([C@H]3CC[C@@H](OC(=O)NN4CCCC4)C3)[nH]n2)c1] |
| 218 | 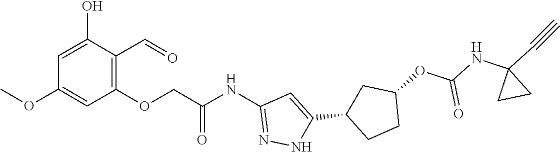 [SMILES: C#CC1(NC(=O)O[C@@H]2CC[C@H](c3cc(NC(=O)COc4cc(OC)cc(O)c4C=O)n[nH]3)C2)CC1] |
| 219 | 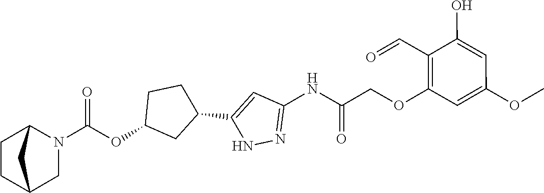 [SMILES: COc1cc(O)c(C=O)c(OCC(=O)Nc2cc([C@H]3CC[C@@H](OC(=O)N4C[C@@H]5CC[C@H]4C5)C3)[nH]n2)c1] |
| 220 | 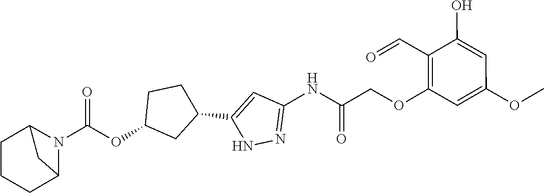 [SMILES: COc1cc(O)c(C=O)c(OCC(=O)Nc2cc([C@H]3CC[C@@H](OC(=O)N4C5CCCC4C5)C3)[nH]n2)c1] |
| 221 | 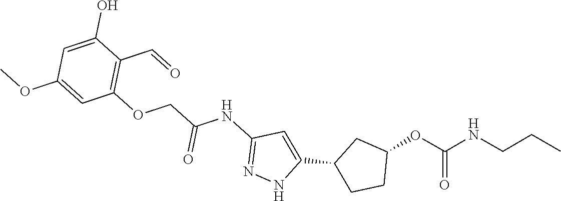 [SMILES: CCCNC(=O)O[C@@H]1CC[C@H](c2cc(NC(=O)COc3cc(OC)cc(O)c3C=O)n[nH]2)C1] |
| 222 | 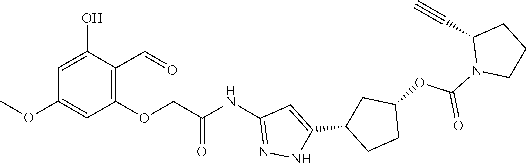 [SMILES: C#C[C@@H]1CCCN1C(=O)O[C@@H]1CC[C@H](c2cc(NC(=O)COc3cc(OC)cc(O)c3C=O)n[nH]2)C1] |
| 223 | 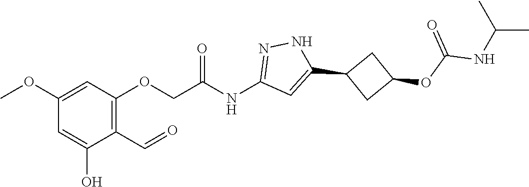 [SMILES: COc1cc(O)c(C=O)c(OCC(=O)Nc2cc([C@H]3C[C@@H](OC(=O)NC(C)C)C3)[nH]n2)c1] |
| 224 | 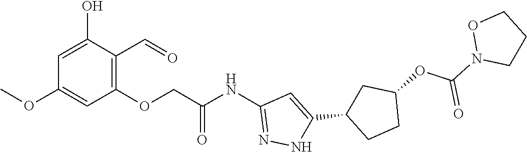 [SMILES: COc1cc(O)c(C=O)c(OCC(=O)Nc2cc([C@H]3CC[C@@H](OC(=O)N4CCCO4)C3)[nH]n2)c1] |
| 225 | 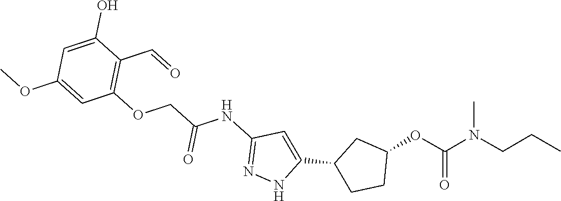 [SMILES: CCCN(C)C(=O)O[C@@H]1CC[C@H](c2cc(NC(=O)COc3cc(OC)cc(O)c3C=O)n[nH]2)C1] |
| 226 | 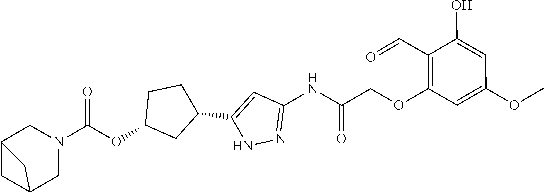 [SMILES: COc1cc(O)c(C=O)c(OCC(=O)Nc2cc([C@H]3CC[C@@H](OC(=O)N4CC5CC(C5)C4)C3)[nH]n2)c1] |
| 227 | 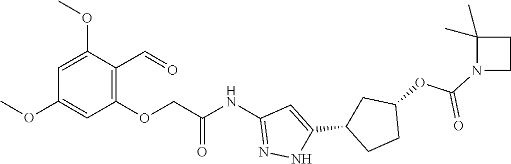 [SMILES: COc1cc(OC)c(C=O)c(OCC(=O)Nc2cc([C@H]3CC[C@@H](OC(=O)N4CCC4(C)C)C3)[nH]n2)c1] |
| 228 | 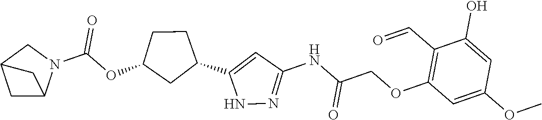 [SMILES: COc1cc(O)c(C=O)c(OCC(=O)Nc2cc([C@H]3CC[C@@H](OC(=O)N4CC5CC4C5)C3)[nH]n2)c1] |
| 229 | 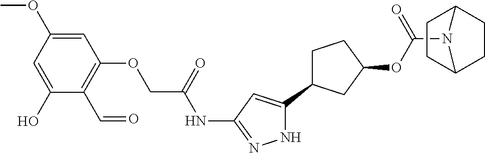 [SMILES: COc1cc(O)c(C=O)c(OCC(=O)Nc2cc([C@H]3CC[C@@H](OC(=O)N4C5CCC4CC5)C3)[nH]n2)c1] |
| 230 | 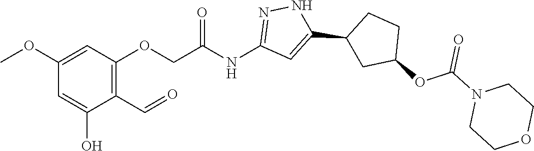 [SMILES: COc1cc(O)c(C=O)c(OCC(=O)Nc2cc([C@H]3CC[C@@H](OC(=O)N4CCOCC4)C3)[nH]n2)c1] |
| 231 | 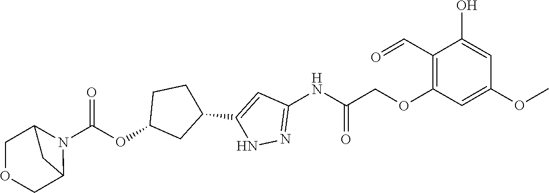 [SMILES: COc1cc(O)c(C=O)c(OCC(=O)Nc2cc([C@H]3CC[C@@H](OC(=O)N4C5COCC4C5)C3)[nH]n2)c1] |
| 232 | 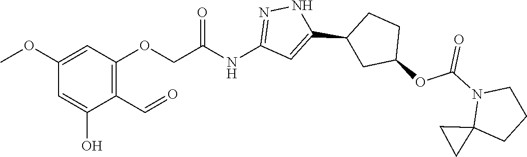 [SMILES: COc1cc(O)c(C=O)c(OCC(=O)Nc2cc([C@H]3CC[C@@H](OC(=O)N4CCCC45CC5)C3)[nH]n2)c1] |
| 233 | 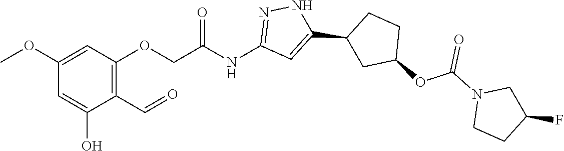 [SMILES: COc1cc(O)c(C=O)c(OCC(=O)Nc2cc([C@H]3CC[C@@H](OC(=O)N4CC[C@H](F)C4)C3)[nH]n2)c1] |
| 234 | 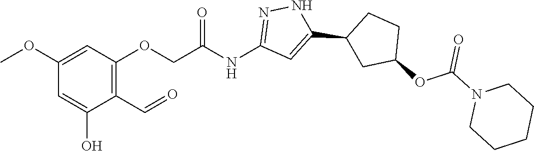 [SMILES: COc1cc(O)c(C=O)c(OCC(=O)Nc2cc([C@H]3CC[C@@H](OC(=O)N4CCCCC4)C3)[nH]n2)c1] |
| 235 | 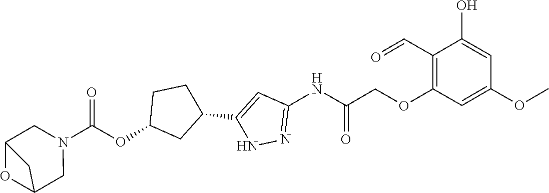 [SMILES: COc1cc(O)c(C=O)c(OCC(=O)Nc2cc([C@H]3CC[C@@H](OC(=O)N4CC5CC(C4)O5)C3)[nH]n2)c1] |
| 236 | 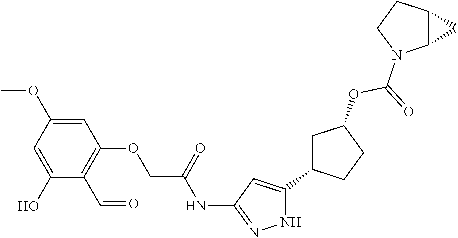 [SMILES: COc1cc(O)c(C=O)c(OCC(=O)Nc2cc([C@H]3CC[C@@H](OC(=O)N4CC[C@@H]5C[C@@H]54)C3)[nH]n2)c1] |
| 237 | 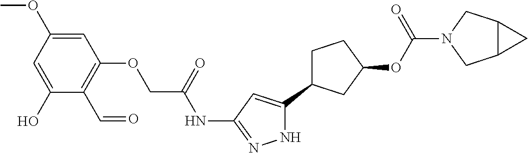 [SMILES: COc1cc(O)c(C=O)c(OCC(=O)Nc2cc([C@H]3CC[C@@H](OC(=O)N4CC5CC5C4)C3)[nH]n2)c1] |
| 238 | 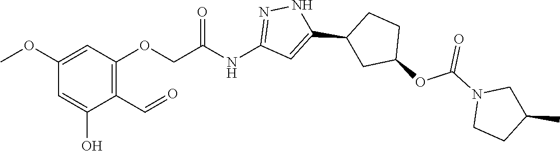 |
| 239 | 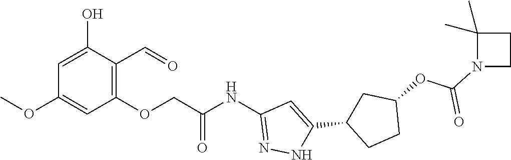 [SMILES: COc1cc(O)c(C=O)c(OCC(=O)Nc2cc([C@H]3CC[C@@H](OC(=O)N4CCC4(C)C)C3)[nH]n2)c1] |
| 240 | 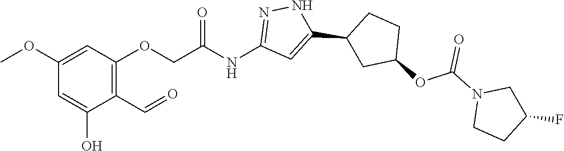 [SMILES: COc1cc(O)c(C=O)c(OCC(=O)Nc2cc([C@H]3CC[C@@H](OC(=O)N4CC[C@@H](F)C4)C3)[nH]n2)c1] |
| 241 | 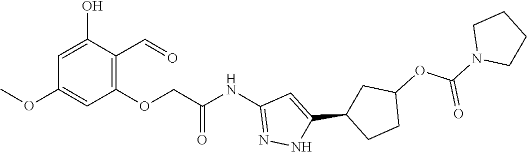 [SMILES: COc1cc(O)c(C=O)c(OCC(=O)Nc2cc([C@@H]3CCC(OC(=O)N4CCCC4)C3)[nH]n2)c1] |
| 242 | 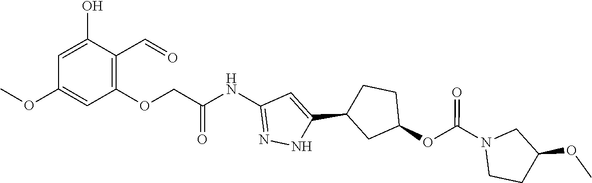 |
| 243 | 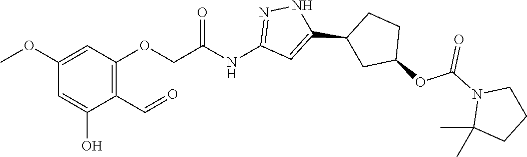 [SMILES: COc1cc(O)c(C=O)c(OCC(=O)Nc2cc([C@H]3CC[C@@H](OC(=O)N4CCCC4(C)C)C3)[nH]n2)c1] |
| 244 | 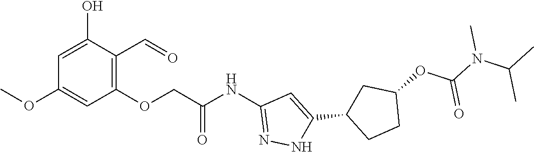 [SMILES: COc1cc(O)c(C=O)c(OCC(=O)Nc2cc([C@H]3CC[C@@H](OC(=O)N(C)C(C)C)C3)[nH]n2)c1] |
| 245 | 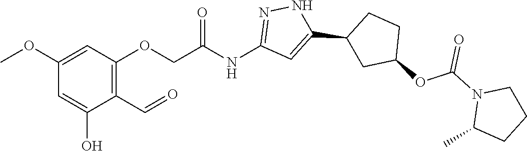 [SMILES: COc1cc(O)c(C=O)c(OCC(=O)Nc2cc([C@H]3CC[C@@H](OC(=O)N4CCC[C@@H]4C)C3)[nH]n2)c1] |
| 246 | 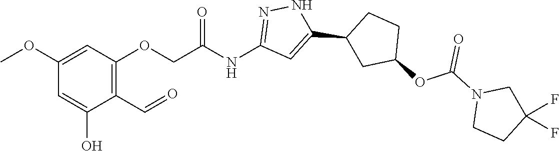 [SMILES: COc1cc(O)c(C=O)c(OCC(=O)Nc2cc([C@H]3CC[C@@H](OC(=O)N4CCC(F)(F)C4)C3)[nH]n2)c1] |
| 247 | 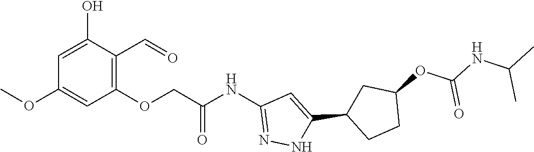 [SMILES: COc1cc(O)c(C=O)c(OCC(=O)Nc2cc([C@@H]3CC[C@H](OC(=O)NC(C)C)C3)[nH]n2)c1] |
| 248 | 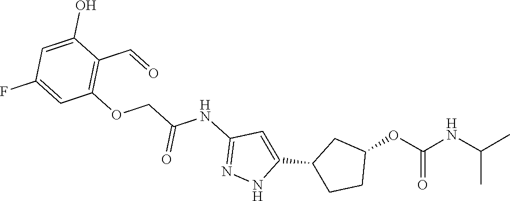 [SMILES: CC(C)NC(=O)O[C@@H]1CC[C@H](c2cc(NC(=O)COc3cc(F)cc(O)c3C=O)n[nH]2)C1] |
| 249 | 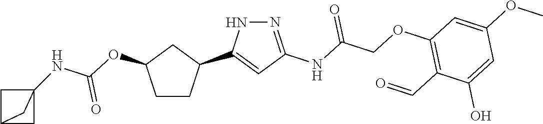 [SMILES: COc1cc(O)c(C=O)c(OCC(=O)Nc2cc([C@H]3CC[C@@H](OC(=O)NC45CC(C4)C5)C3)[nH]n2)c1] |
| 250 | 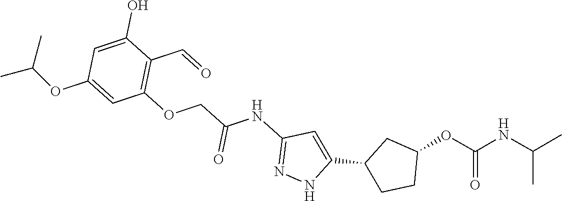 [SMILES: CC(C)NC(=O)O[C@@H]1CC[C@H](c2cc(NC(=O)COc3cc(OC(C)C)cc(O)c3C=O)n[nH]2)C1] |
| 251 | 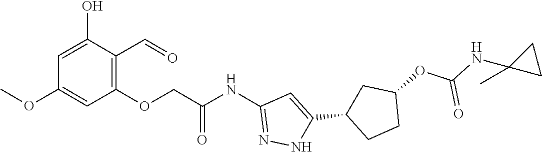 [SMILES: COc1cc(O)c(C=O)c(OCC(=O)Nc2cc([C@H]3CC[C@@H](OC(=O)NC4(C)CC4)C3)[nH]n2)c1] |
| 252 | 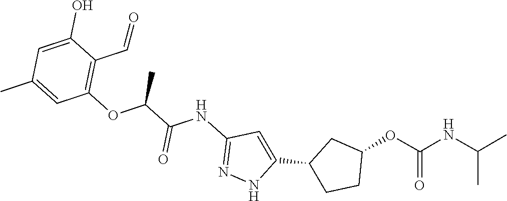 [SMILES: Cc1cc(O)c(C=O)c(O[C@@H](C)C(=O)Nc2cc([C@H]3CC[C@@H](OC(=O)NC(C)C)C3)[nH]n2)c1] |
| 253 | 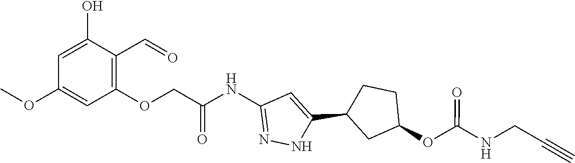 [SMILES: C#CCNC(=O)O[C@@H]1CC[C@H](c2cc(NC(=O)COc3cc(OC)cc(O)c3C=O)n[nH]2)C1] |
| 254 | 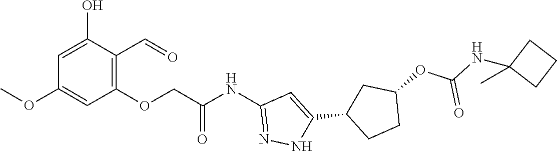 [SMILES: COc1cc(O)c(C=O)c(OCC(=O)Nc2cc([C@H]3CC[C@@H](OC(=O)NC4(C)CCC4)C3)[nH]n2)c1] |
| 255 | 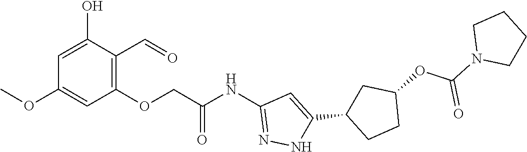 [SMILES: COc1cc(O)c(C=O)c(OCC(=O)Nc2cc([C@H]3CC[C@@H](OC(=O)N4CCCC4)C3)[nH]n2)c1] |
| 256 | 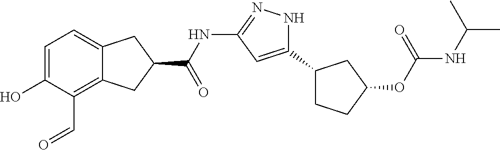 [SMILES: CC(C)NC(=O)O[C@@H]1CC[C@H](c2cc(NC(=O)[C@@H]3Cc4ccc(O)c(C=O)c4C3)n[nH]2)C1] |
| 257 | 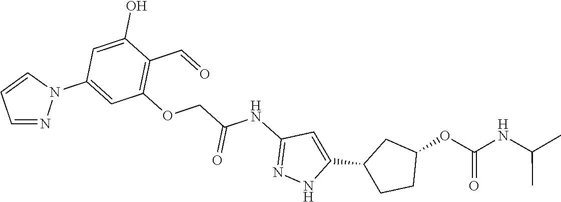 [SMILES: CC(C)NC(=O)O[C@@H]1CC[C@H](c2cc(NC(=O)COc3cc(-n4cccn4)cc(O)c3C=O)n[nH]2)C1] |
| 258 | 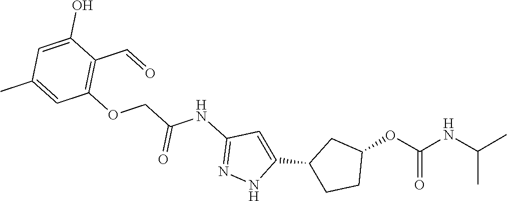 [SMILES: Cc1cc(O)c(C=O)c(OCC(=O)Nc2cc([C@H]3CC[C@@H](OC(=O)NC(C)C)C3)[nH]n2)c1] |
| 259 | 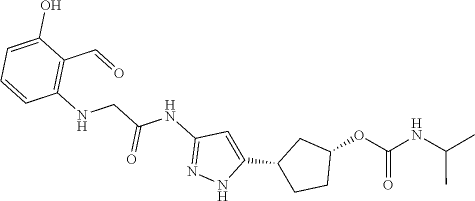 [SMILES: CC(C)NC(=O)O[C@@H]1CC[C@H](c2cc(NC(=O)CNc3cccc(O)c3C=O)n[nH]2)C1] |
| 260 | 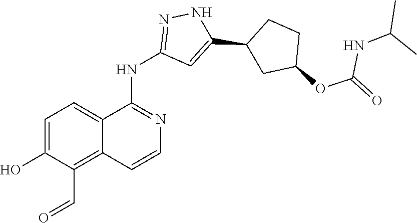 [SMILES: CC(C)NC(=O)O[C@@H]1CC[C@H](c2cc(Nc3nccc4c(C=O)c(O)ccc34)n[nH]2)C1] |
| 261 | 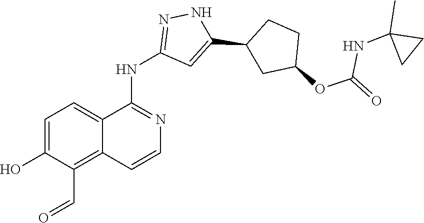 [SMILES: CC1(NC(=O)O[C@@H]2CC[C@H](c3cc(Nc4nccc5c(C=O)c(O)ccc45)n[nH]3)C2)CC1] |
| 262 | 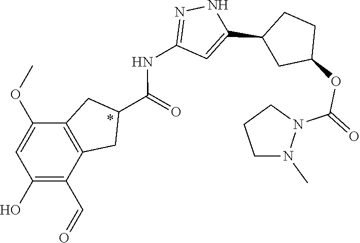 [SMILES: COc1cc(O)c(C=O)c2c1CC(C(=O)Nc1cc([C@H]3CC[C@@H](OC(=O)N4CCCN4C)C3)[nH]n1)C2] |
| 263 | 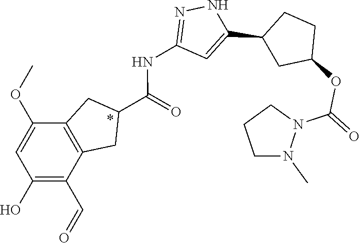 [SMILES: COc1cc(O)c(C=O)c2c1CC(C(=O)Nc1cc([C@H]3CC[C@@H](OC(=O)N4CCCN4C)C3)[nH]n1)C2] |
| 264 | 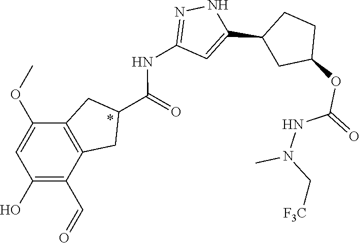 [SMILES: COc1cc(O)c(C=O)c2c1CC(C(=O)Nc1cc([C@H]3CC[C@@H](OC(=O)NN(C)CC(F)(F)F)C3)[nH]n1)C2] |
| 265 | 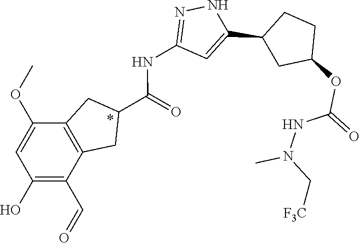 [SMILES: COc1cc(O)c(C=O)c2c1CC(C(=O)Nc1cc([C@H]3CC[C@@H](OC(=O)NN(C)CC(F)(F)F)C3)[nH]n1)C2] |
| 266 | 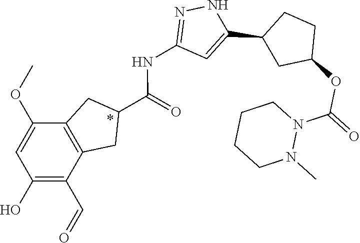 [SMILES: COc1cc(O)c(C=O)c2c1CC(C(=O)Nc1cc([C@H]3CC[C@@H](OC(=O)N4CCCCN4C)C3)[nH]n1)C2] |
| 267 | 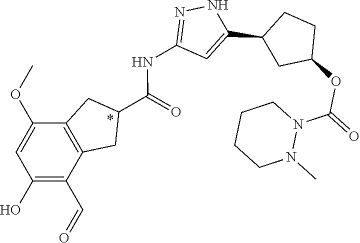 [SMILES: COc1cc(O)c(C=O)c2c1CC(C(=O)Nc1cc([C@H]3CC[C@@H](OC(=O)N4CCCCN4C)C3)[nH]n1)C2] |
| 268 | 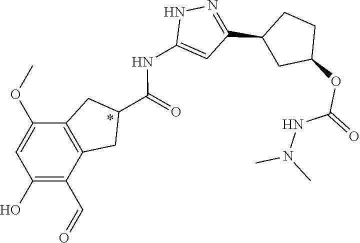 [SMILES: COc1cc(O)c(C=O)c2c1CC(C(=O)Nc1cc([C@H]3CC[C@@H](OC(=O)NN(C)C)C3)n[nH]1)C2] |
| 269 | 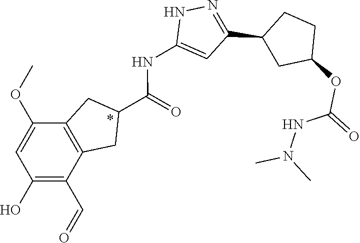 [SMILES: COc1cc(O)c(C=O)c2c1CC(C(=O)Nc1cc([C@H]3CC[C@@H](OC(=O)NN(C)C)C3)n[nH]1)C2] |
| 270 | 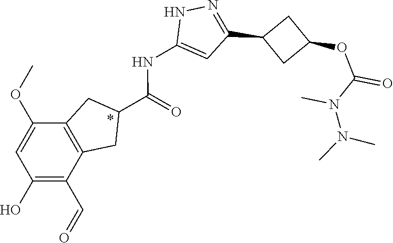 [SMILES: COc1cc(O)c(C=O)c2c1CC(C(=O)Nc1cc([C@H]3C[C@@H](OC(=O)N(C)N(C)C)C3)n[nH]1)C2] |
| 271 | 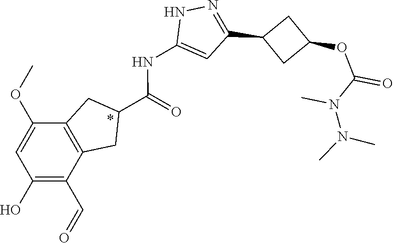 [SMILES: COc1cc(O)c(C=O)c2c1CC(C(=O)Nc1cc([C@H]3C[C@@H](OC(=O)N(C)N(C)C)C3)n[nH]1)C2] |
| 272 | 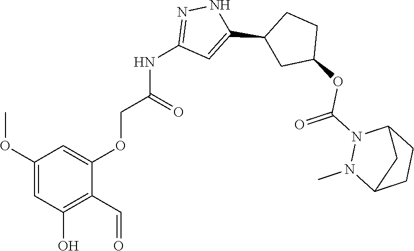 [SMILES: COc1cc(O)c(C=O)c(OCC(=O)Nc2cc([C@H]3CC[C@@H](OC(=O)N4C5CCC(C5)N4C)C3)[nH]n2)c1] |
| 273 | 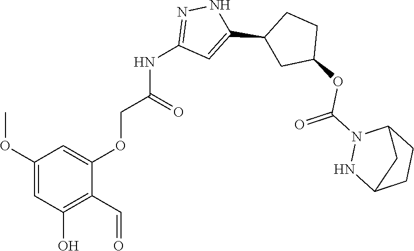 [SMILES: COc1cc(O)c(C=O)c(OCC(=O)Nc2cc([C@H]3CC[C@@H](OC(=O)N4NC5CCC4C5)C3)[nH]n2)c1] |
| 274 | 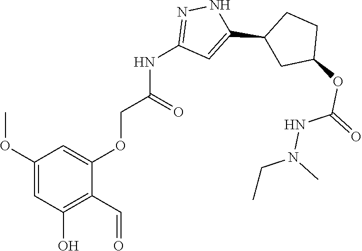 [SMILES: CCN(C)NC(=O)O[C@@H]1CC[C@H](c2cc(NC(=O)COc3cc(OC)cc(O)c3C=O)n[nH]2)C1] |
| 275 | 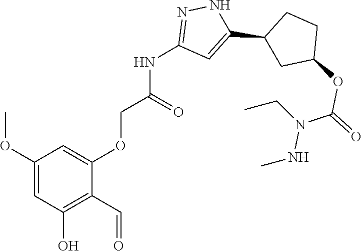 [SMILES: CCN(NC)C(=O)O[C@@H]1CC[C@H](c2cc(NC(=O)COc3cc(OC)cc(O)c3C=O)n[nH]2)C1] |
| 276 | 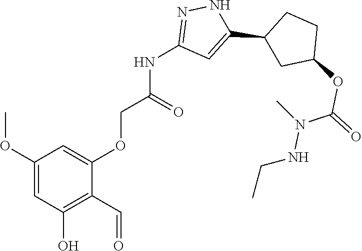 [SMILES: CCNN(C)C(=O)O[C@@H]1CC[C@H](c2cc(NC(=O)COc3cc(OC)cc(O)c3C=O)n[nH]2)C1] |
| 277 | 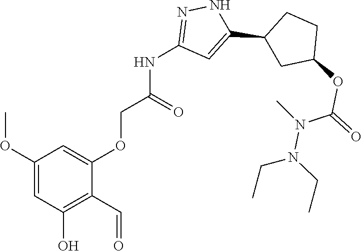 [SMILES: CCN(CC)N(C)C(=O)O[C@@H]1CC[C@H](c2cc(NC(=O)COc3cc(OC)cc(O)c3C=O)n[nH]2)C1] |
| 278 | 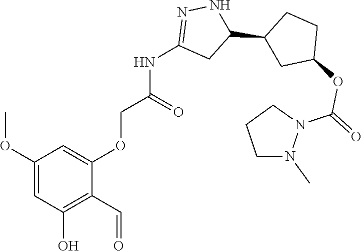 [SMILES: COc1cc(O)c(C=O)c(OCC(=O)NC2=NNC([C@H]3CC[C@@H](OC(=O)N4CCCN4C)C3)C2)c1] |
| 279 | 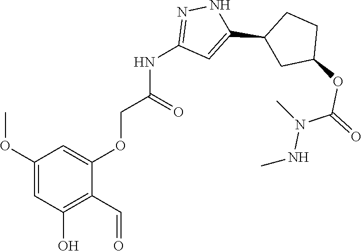 [SMILES: CNN(C)C(=O)O[C@@H]1CC[C@H](c2cc(NC(=O)COc3cc(OC)cc(O)c3C=O)n[nH]2)C1] |
| 280 | 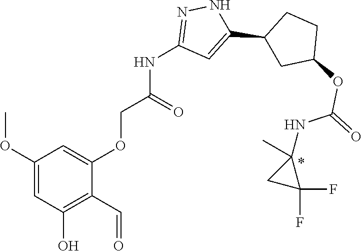 [SMILES: COc1cc(O)c(C=O)c(OCC(=O)Nc2cc([C@H]3CC[C@@H](OC(=O)NC4(C)CC4(F)F)C3)[nH]n2)c1] |
| 281 | 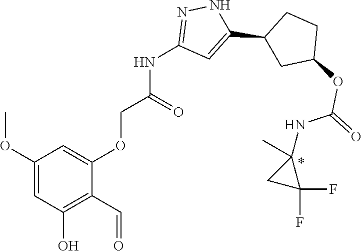 [SMILES: COc1cc(O)c(C=O)c(OCC(=O)Nc2cc([C@H]3CC[C@@H](OC(=O)NC4(C)CC4(F)F)C3)[nH]n2)c1] |
| 282 | 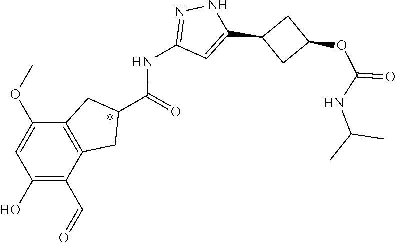 [SMILES: COc1cc(O)c(C=O)c2c1CC(C(=O)Nc1cc([C@H]3C[C@@H](OC(=O)NC(C)C)C3)[nH]n1)C2] |
| 283 | 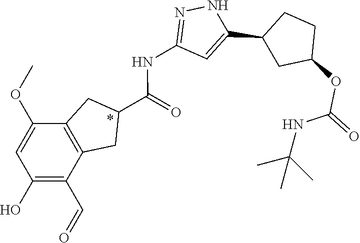 [SMILES: COc1cc(O)c(C=O)c2c1CC(C(=O)Nc1cc([C@H]3CC[C@@H](OC(=O)NC(C)(C)C)C3)[nH]n1)C2] |
| 284 | 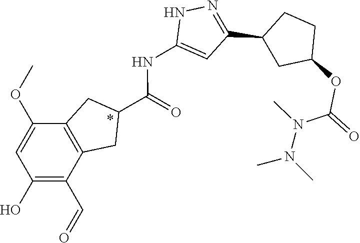 [SMILES: COc1cc(O)c(C=O)c2c1CC(C(=O)Nc1cc([C@H]3CC[C@@H](OC(=O)N(C)N(C)C)C3)n[nH]1)C2] |

In some embodiments, for the compound or salt of Formula (I), (II), (II-A), (II-B), (II-C), (II-D), (II-E), or (II-F), is selected from a compound in Table 1A.

TABLE 1A
Selected CDK2 Prodrug Compounds.

| # | Compound |
|---|---|
| 128b | 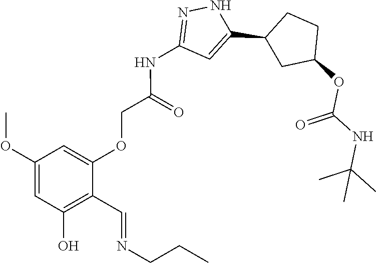 [SMILES: CCC/N=C/c1c(O)cc(OC)cc1OCC(=O)Nc1cc([C@H]2CC[C@@H](OC(=O)NC(C)(C)C)C2)[nH]n1] |
| 64d | 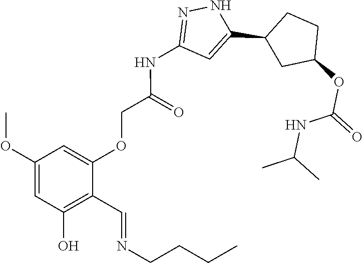 [SMILES: CCCC/N=C/c1c(O)cc(OC)cc1OCC(=O)Nc1cc([C@H]2CC[C@@H](OC(=O)NC(C)C)C2)[nH]n1] |
| 172a | 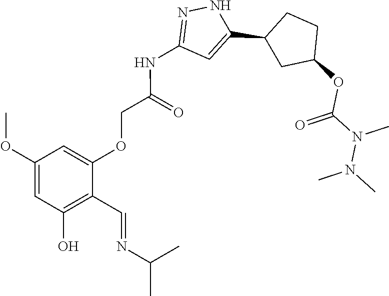 [SMILES: COc1cc(O)c(/C=N/C(C)C)c(OCC(=O)Nc2cc([C@H]3CC[C@@H](OC(=O)N(C)N(C)C)C3)[nH]n2)c1] |
| 64e | 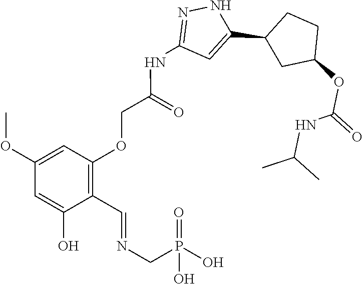 [SMILES: COc1cc(O)c(/C=N/CP(=O)(O)O)c(OCC(=O)Nc2cc([C@H]3CC[C@@H](OC(=O)NC(C)C)C3)[nH]n2)c1] |
| 251b | 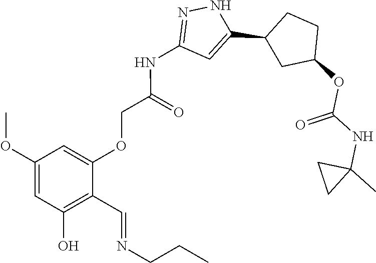 [SMILES: CCC/N=C/c1c(O)cc(OC)cc1OCC(=O)Nc1cc([C@H]2CC[C@@H](OC(=O)NC3(C)CC3)C2)[nH]n1] |
| 145a | 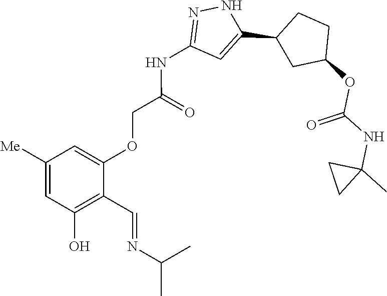 [SMILES: Cc1cc(O)c(/C=N/C(C)C)c(OCC(=O)Nc2cc([C@H]3CC[C@@H](OC(=O)NC4(C)CC4)C3)[nH]n2)c1] |
| 251d | 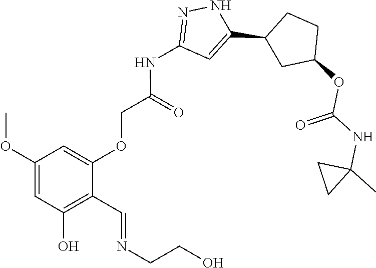 [SMILES: COc1cc(O)c(/C=N/CCO)c(OCC(=O)Nc2cc([C@H]3CC[C@@H](OC(=O)NC4(C)CC4)C3)[nH]n2)c1] |
| 64f | 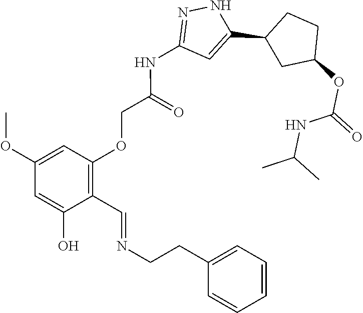 [SMILES: COc1cc(O)c(/C=N/CCc2ccccc2)c(OCC(=O)Nc2cc([C@H]3CC[C@@H](OC(=O)NC(C)C)C3)[nH]n2)c1] |
| 251c | 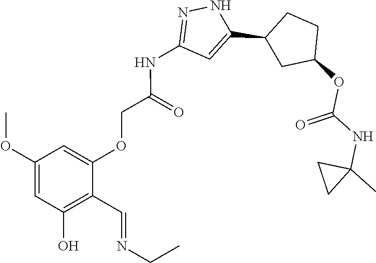 [SMILES: CC/N=C/c1c(O)cc(OC)cc1OCC(=O)Nc1cc([C@H]2CC[C@@H](OC(=O)NC3(C)CC3)C2)[nH]n1] |
| 64g | 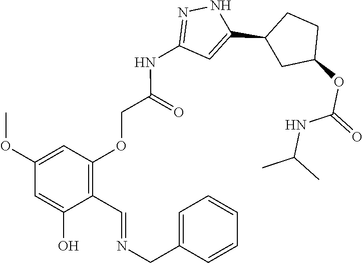 [SMILES: COc1cc(O)c(/C=N/Cc2ccccc2)c(OCC(=O)Nc2cc([C@H]3CC[C@@H](OC(=O)NC(C)C)C3)[nH]n2)c1] |
| 64h | 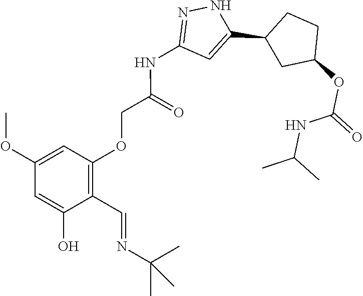 [SMILES: COc1cc(O)c(/C=N/C(C)(C)C)c(OCC(=O)Nc2cc([C@H]3CC[C@@H](OC(=O)NC(C)C)C3)[nH]n2)c1] |
| 64m | 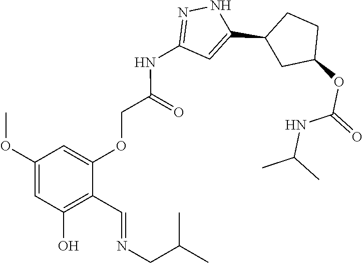 [SMILES: COc1cc(O)c(/C=N/CC(C)C)c(OCC(=O)Nc2cc([C@H]3CC[C@@H](OC(=O)NC(C)C)C3)[nH]n2)c1] |
| 64i | 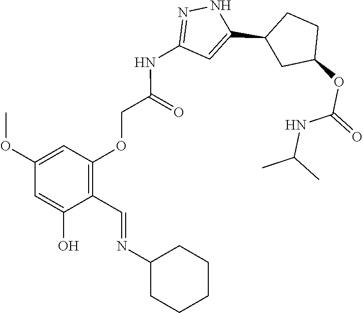 [SMILES: COc1cc(O)c(/C=N/C2CCCCC2)c(OCC(=O)Nc2cc([C@H]3CC[C@@H](OC(=O)NC(C)C)C3)[nH]n2)c1] |
| 64n | 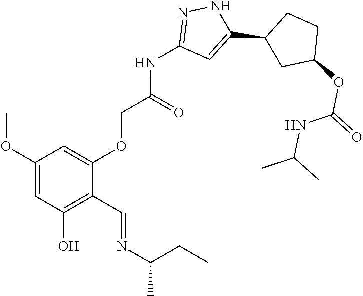 [SMILES: CC[C@H](C)/N=C/c1c(O)cc(OC)cc1OCC(=O)Nc1cc([C@H]2CC[C@@H](OC(=O)NC(C)C)C2)[nH]n1] |
| 64j | 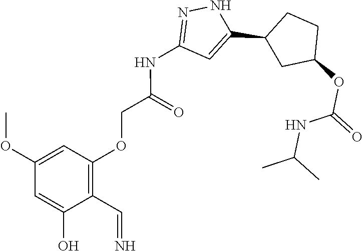 [SMILES: COc1cc(O)c(C=N)c(OCC(=O)Nc2cc([C@H]3CC[C@@H](OC(=O)NC(C)C)C3)[nH]n2)c1] |
| 64o | 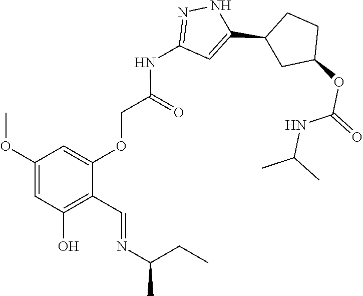 [SMILES: CC[C@@H](C)/N=C/c1c(O)cc(OC)cc1OCC(=O)Nc1cc([C@H]2CC[C@@H](OC(=O)NC(C)C)C2)[nH]n1] |
| 64k | 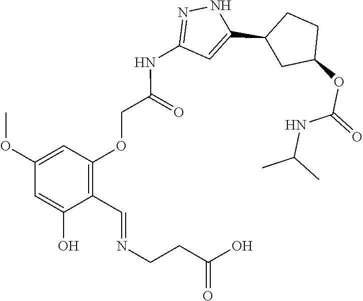 [SMILES: COc1cc(O)c(/C=N/CCC(=O)O)c(OCC(=O)Nc2cc([C@H]3CC[C@@H](OC(=O)NC(C)C)C3)[nH]n2)c1] |
| 128a | 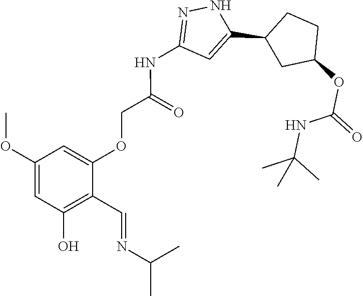 [SMILES: COc1cc(O)c(/C=N/C(C)C)c(OCC(=O)Nc2cc([C@H]3CC[C@@H](OC(=O)NC(C)(C)C)C3)[nH]n2)c1] |
| 64l | 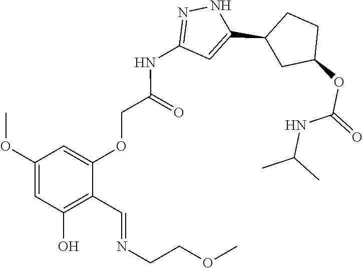 [SMILES: COCC/N=C/c1c(O)cc(OC)cc1OCC(=O)Nc1cc([C@H]2CC[C@@H](OC(=O)NC(C)C)C2)[nH]n1] |
| 254a | 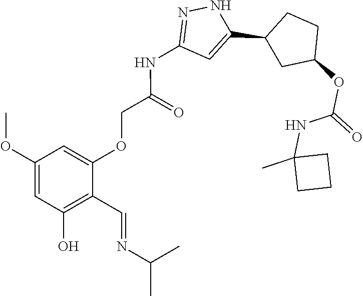 [SMILES: COc1cc(O)c(/C=N/C(C)C)c(OCC(=O)Nc2cc([C@H]3CC[C@@H](OC(=O)NC4(C)CCC4)C3)[nH]n2)c1] |
| 64b | 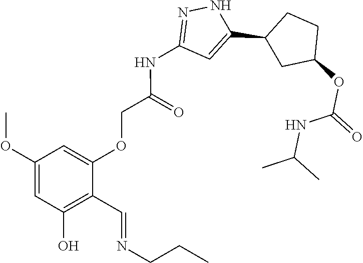 [SMILES: CCC/N=C/c1c(O)cc(OC)cc1OCC(=O)Nc1cc([C@H]2CC[C@@H](OC(=O)NC(C)C)C2)[nH]n1] |
| 64c | 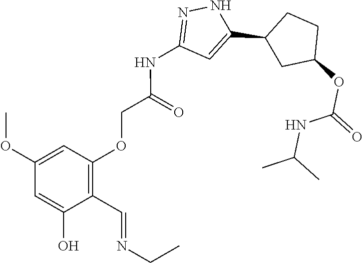 [SMILES: CC/N=C/c1c(O)cc(OC)cc1OCC(=O)Nc1cc([C@H]2CC[C@@H](OC(=O)NC(C)C)C2)[nH]n1] |
| 180a | 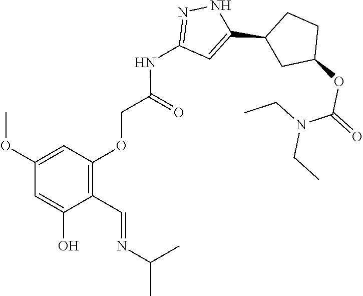 [SMILES: CCN(CC)C(=O)O[C@@H]1CC[C@H](c2cc(NC(=O)COc3cc(OC)cc(O)c3/C=N/C(C)C)n[nH]2)C1] |
| 64p | 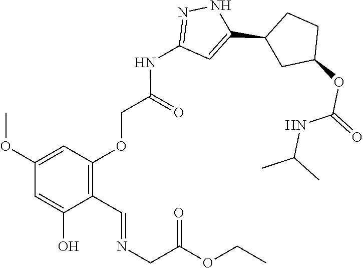 [SMILES: CCOC(=O)C/N=C/c1c(O)cc(OC)cc1OCC(=O)Nc1cc([C@H]2CC[C@@H](OC(=O)NC(C)C)C2)[nH]n1] |
| 253a | 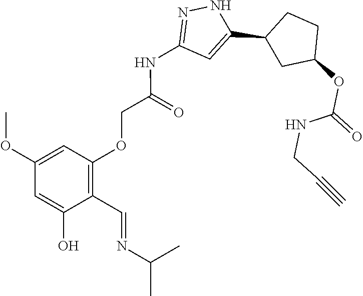 [SMILES: C#CCNC(=O)O[C@@H]1CC[C@H](c2cc(NC(=O)COc3cc(OC)cc(O)c3/C=N/C(C)C)n[nH]2)C1] |
| 64q | 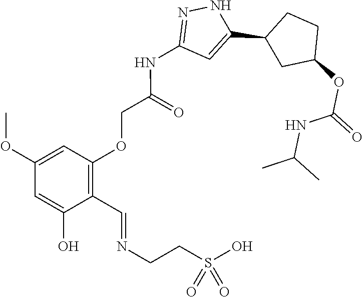 [SMILES: COc1cc(O)c(/C=N/CCS(=O)(=O)O)c(OCC(=O)Nc2cc([C@H]3CC[C@@H](OC(=O)NC(C)C)C3)[nH]n2)c1] |
| 82a | 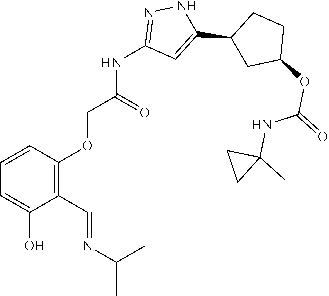 [SMILES: CC(C)/N=C/c1c(O)cccc1OCC(=O)Nc1cc([C@H]2CC[C@@H](OC(=O)NC3(C)CC3)C2)[nH]n1] |
| 235a | 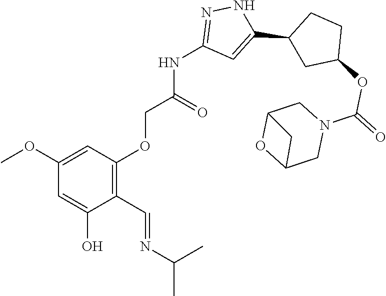 [SMILES: COc1cc(O)c(/C=N/C(C)C)c(OCC(=O)Nc2cc([C@H]3CC[C@@H](OC(=O)N4CC5CC(C4)O5)C3)[nH]n2)c1] |
| 221a | 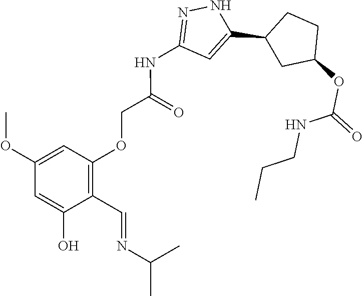 [SMILES: CCCNC(=O)O[C@@H]1CC[C@H](c2cc(NC(=O)COc3cc(OC)cc(O)c3/C=N/C(C)C)n[nH]2)C1] |
| 224a | 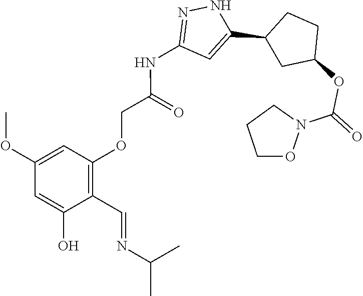 [SMILES: COc1cc(O)c(/C=N/C(C)C)c(OCC(=O)Nc2cc([C@H]3CC[C@@H](OC(=O)N4CCCO4)C3)[nH]n2)c1] |
| 223a | 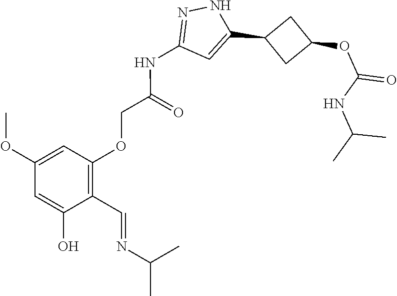 [SMILES: COc1cc(O)c(/C=N/C(C)C)c(OCC(=O)Nc2cc([C@H]3C[C@@H](OC(=O)NC(C)C)C3)[nH]n2)c1] |
| 251a | 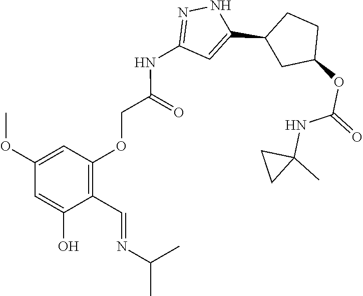 [SMILES: COc1cc(O)c(/C=N/C(C)C)c(OCC(=O)Nc2cc([C@H]3CC[C@@H](OC(=O)NC4(C)CC4)C3)[nH]n2)c1] |
| 283a | 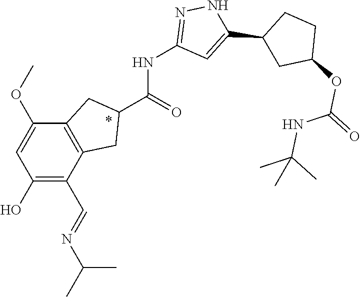 [SMILES: COc1cc(O)c(/C=N/C(C)C)c2c1CC(C(=O)Nc1cc([C@H]3CC[C@@H](OC(=O)NC(C)(C)C)C3)[nH]n1)C2] |
| 64a | 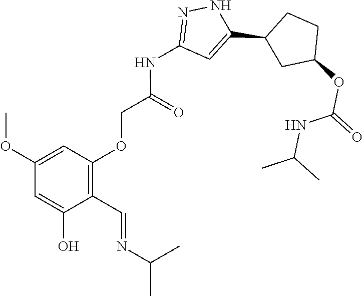 [SMILES: COc1cc(O)c(/C=N/C(C)C)c(OCC(=O)Nc2cc([C@H]3CC[C@@H](OC(=O)NC(C)C)C3)[nH]n2)c1] |
| 284a | 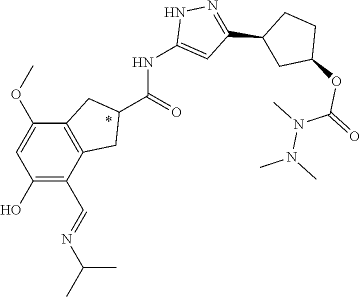 [SMILES: COc1cc(O)c(/C=N/C(C)C)c2c1CC(C(=O)Nc1cc([C@H]3CC[C@@H](OC(=O)N(C)N(C)C)C3)n[nH]1)C2] |
| 231a | 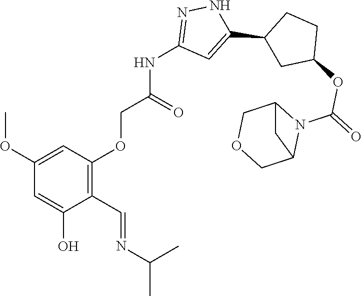 [SMILES: COc1cc(O)c(/C=N/C(C)C)c(OCC(=O)Nc2cc([C@H]3CC[C@@H](OC(=O)N4C5COCC4C5)C3)[nH]n2)c1] |
| 284c | 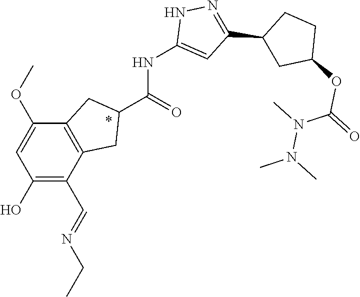 [SMILES: CC/N=C/c1c(O)cc(OC)c2c1CC(C(=O)Nc1cc([C@H]3CC[C@@H](OC(=O)N(C)N(C)C)C3)n[nH]1)C2] |
| 284b | 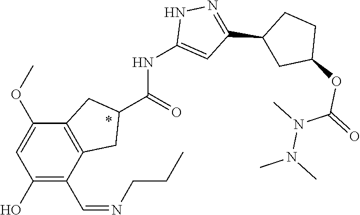 [SMILES: CCC/N=C\c1c(O)cc(OC)c2c1CC(C(=O)Nc1cc([C@H]3CC[C@@H](OC(=O)N(C)N(C)C)C3)n[nH]1)C2] |

*Denotes a stereocenter with an undetermined absolute stereochemistry of a single isomer.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (I), (II), (II-A), (II-B), (II-C), (II-D)), (II-E), (II-F), (III), (IV), (IV-A), (IV-B), (IV-C), or (V), are intended to include all Z—, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), (II), (II-A), (II-B), (II-C), (II-D), (II-E), (II-F), (III), (IV), (IV-A), (IV-B), (IV-C), or (V), may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), (II), (II-A), (II-B), (II-C), (II-D), (II-E), (II-F), (III), (IV), (IV-A), (IV-B), (IV-C), or (V), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), (II), (II-A), (II-B), (II-C), (II-D), (II-E), (II-F), (III), (IV), (IV-A), (IV-B), (IV-C), or (V), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

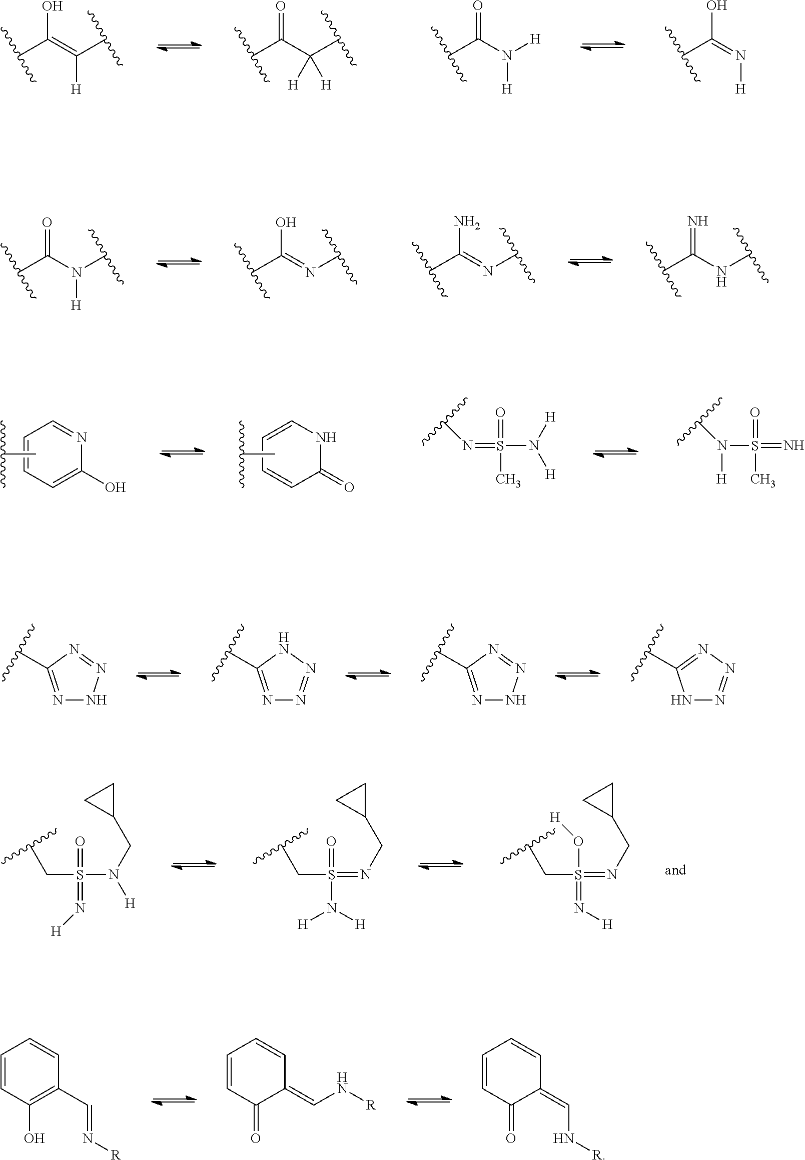

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I), (II), (II-A), (II-B), (II-C), (II-D), (II-E), (II-F), (III), (IV), (IV-A), (IV-B), (IV-C), or (V). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formula (I), (II), (II-A), (II-B), (II-C), (II-D), (II-E), (II-F), (III), (IV), (IV-A), (IV-B), (IV-C), or (V), include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formula (I), (II), (II-A), (II-B), (II-C), (II-D), (II-E), (II-F), (III), (IV), (IV-A), (IV-B), (IV-C), or (V), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The term “prodrug” as used herein, is intended to encompass compounds which, under physiologic conditions, are converted into pharmaceutical agents of the present disclosure. In some embodiments, term “prodrug”, is intended to encompass compounds which, under physiologic conditions, are converted into pharmaceutical agents of compounds or salts of Formula (I), (II), (II-A), (II-B), (II-C), (II-D)), (II-E), (II-F), (III), (IV), (IV-A), (IV-B), (IV-C), or (V). In certain embodiments, compounds or salts of Formula (I), (II), (II-A), (II-B), (II-C), (II-D), (II-E), (II-F), (III), (IV), (IV-A), (IV-B), (IV-C), or (V), may be prodrugs, e.g., wherein a hydroxyl in the parent compound is presented as an ester or a carbonate, a carboxylic acid is presented as an ester, or an aldehyde is presented as an imine. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal.

In certain embodiments, compounds or salts of Formula (I), (II), (II-A), (II-B), (II-C), (II-D), (II-E), (II-F), (III), (IV), (IV-A), (IV-B), (IV-C), or (V), may be prodrugs, wherein the parent compound comprises of an aldehyde that may be presented as an imine. For example, the imine prodrug may be hydrolyzed enzymatically or chemically to furnish the parent aldehyde. As disclosed herein, imines are the preferred prodrugs of the present disclosure. In certain embodiments, imines are preferred prodrugs for compounds or salts of Formula (I), (II), (II-A), (II-B), (II-C), (II-D)), (II-E), (II-F), (III), (IV), (IV-A), (IV-B), (IV-C), or (V).

In certain embodiments, compounds or salts of Formula (I), (II), (II-A), (II-B), (II-C), (II-D), (II-E), (II-F), (III), (IV), (IV-A), (IV-B), (IV-C), or (V), may be prodrugs, e.g., wherein a hydroxyl in the parent compound is presented as an ester or a carbonate, or carboxylic acid present in the parent compound is presented as an ester. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I), (II), (II-A), (II-B), (II-C), (II-D), (II-E), (II-F), (III), (IV), (IV-A), (IV-B), (IV-C), or (V), and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

A compound or salt of any one of Formula (I), (II), (II-A), (II-B), (II-C), (II-D), (II-E), (II-F), (III), (IV), (IV-A), (IV-B), (IV-C), or (V), may be formulated in any suitable pharmaceutical formulation. A pharmaceutical formulation of the present disclosure typically contains an active ingredient (e.g., compound or salt of any one of Formula (I), (II), (II-A), (II-B), (II-C), (II-D), (II-E), (II-F), (III), (IV), (IV-A), (IV-B), (IV-C), or (V), and one or more pharmaceutically acceptable excipients or carriers, including but not limited to: inert solid diluents and fillers, diluents, sterile aqueous solution and various organic solvents, permeation enhancers, antioxidants, solubilizers, and adjuvants.

Pharmaceutical compositions may also be prepared from a compound or salt of any one of Formula (I), (II), (II-A), (II-B), (II-C), (II-D), (II-E), (II-F), (III), (IV), (IV-A), (IV-B), (IV-C), or (V), and one or more pharmaceutically acceptable excipients. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some aspects, the present disclosure provides a method for treatment, comprising administering to a subject in need thereof an effective amount of a compound or salt of Formula (I), (II), (II-A), (II-B), (II-C), (II-D)), (II-E), (II-F), (III), (IV), (IV-A), (IV-B), (IV-C), or (V).

In certain embodiments, the present disclosure provides a method of modulating CDK2. In some embodiments, the method of modulating CDK2 comprises administering to a subject in need thereof a compound or salt of Formula (I), (II), (II-A), (II-B), (II-C), (II-D), (II-E), (II-F), (III), (IV), (IV-A), (IV-B), (IV-C), or (V), or a pharmaceutical composition thereof. In some embodiments, the administering is selective for CDK2 over a CDK selected from CDK1, CDK4, and CDK6. In some embodiments, the administering is selective for CDK2 over CDK1. In some embodiments, the administering is selective for CDK2 over CDK4. In some embodiments, the administering is selective for CDK2 over CDK6.

In certain embodiments, the present disclosure provides a method of inhibiting CDK2. In some embodiments, the method of inhibiting CDK2 comprises administering to a subject in need thereof a compound or salt of Formula (I), (II), (II-A), (II-B), (II-C), (II-D), (II-E), (II-F), (III), (IV), (IV-A), (IV-B), (IV-C), or (V), or a pharmaceutical composition thereof. In some embodiments, the administering is selective for CDK2 over a CDK selected from CDK1, CDK4, and CDK6. In some embodiments, the administering is selective for CDK2 over CDK1. In some embodiments, the administering is selective for CDK2 over CDK4. In some embodiments, the administering is selective for CDK2 over CDK6.

In certain embodiments, the present disclosure provides a method of treating a disease or disorder mediated by CDK2. In some embodiments, the method of treating a disease or disorder mediated by CDK2 comprises administering to a subject in need thereof a compound or salt of Formula (I), (II), (II-A), (II-B), (II-C), (II-D)), (II-E), (II-F), (III), (IV), (IV-A), (IV-B), (IV-C), or (V), or a pharmaceutical composition thereof. In some embodiments, the disease or disorder is cancer. In some embodiments, the disease or disorder is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, and thyroid cancer. In some embodiments, the administering is selective for CDK2 over a CDK selected from CDK1, CDK4, and CDK6. In some embodiments, the administering is selective for CDK2 over CDK1. In some embodiments, the administering is selective for CDK2 over CDK4. In some embodiments, the administering is selective for CDK2 over CDK6.

In certain embodiments, the present disclosure provides a method of inhibiting cell proliferation. In some embodiments, the method of inhibiting cell proliferation comprises administering to a subject in need thereof a compound or salt of Formula (I), (II), (II-A), (II-B), (II-C), (II-D), (II-E), (II-F), (III), (IV), (IV-A), (IV-B), (IV-C), or (V), or a pharmaceutical composition thereof. In some embodiments, the cell proliferation is cancer cell proliferation. In some embodiments, the cancer cell proliferation is selected from breast cancer proliferation, ovarian cancer proliferation, bladder cancer proliferation, uterine cancer proliferation, prostate cancer proliferation, lung cancer proliferation, esophageal cancer proliferation, head and neck cancer proliferation, colorectal cancer proliferation, kidney cancer proliferation, liver cancer proliferation, pancreatic cancer proliferation, stomach cancer proliferation, and thyroid cancer proliferation. In some embodiments, the administering is selective for CDK2 over a CDK selected from CDK1, CDK4, and CDK6. In some embodiments, the administering is selective for CDK2 over CDK1. In some embodiments, the administering is selective for CDK2 over CDK4. In some embodiments, the administering is selective for CDK2 over CDK6.

In certain embodiments, the present disclosure provides a method of treating cancer. In some embodiments, the method of treating cancer comprises administering to a subject in need thereof a compound or salt of Formula (I), (II), (II-A), (II-B), (II-C), (II-D), (II-E), (II-F), (III), (IV), (IV-A), (IV-B), (IV-C), or (V), or a pharmaceutical composition thereof. In some embodiments, the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, and thyroid cancer. In some embodiments, the administering is selective for CDK2 over a CDK selected from CDK1, CDK4, and CDK6. In some embodiments, the administering is selective for CDK2 over CDK1. In some embodiments, the administering is selective for CDK2 over CDK4. In some embodiments, the administering is selective for CDK2 over CDK6.

### CDK2 Protein

In some aspects, the present disclosure provides a CDK2 protein covalently bound to a compound, wherein the compound is covalently bound to a lysine residue of the CDK2 protein. In some embodiments, the compound is exogenous. In some embodiments, the exogenous compound is selected from an exogenous CDK2 inhibitor and an exogenous CDK2 activator. In some embodiments, the exogenous compound is an exogenous CDK2 modulator. In some embodiments, the exogenous compound is an exogenous CDK2 inhibitor.

In some embodiments, the CDK2 protein is selected from a wild-type CDK2 protein and a mutated CDK2 protein. In some embodiments, the CDK2 protein is a mutated CDK2 protein. In some embodiments, the CDK2 protein is a wild-type CDK2 protein.

In some embodiments, the exogenous compound is in contact with a lysine residue of the CDK2 protein as described herein. In some embodiments, the contact is between the lysine reside of the CDK2 protein and the exogenous compound is a covalent bond. In some embodiments, the lysine reside is selected from K6, K9, K20, K24, K33, K34, K56, K65, K88, K89, K105, K129, K142, K178, K237, K242, K250, K273, K278, and K291. In some embodiments, the lysine residue is selected from K20, K33, K89, K129, K178, and K278. In some embodiments, the lysine residue is K89. In some embodiments, the lysine residue is selected from K20, K33, K129, K178, and K278. In some embodiments, the lysine residue is K20. In some embodiments, the lysine residue is K33. In some embodiments, the lysine residue is K129. In some embodiments, the lysine residue is K178. In some embodiments, the lysine residue is K278.

In some embodiments, the covalent bond between the exogenous compound and the lysine residue is a reversible covalent bond. In some embodiments, the reversible covalent bond is selected from a single bond, a double bound, and a triple bond. In some embodiments, the reversible covalent bond is a single bond. In some embodiments the reversible covalent bond is a double bond. In some embodiments, the reversible covalent bond is a triple bond. In some embodiments, the reversible covalent bond is a double bond between a carbon atom on the exogenous compound and the nitrogen atom on the sidechain of the lysine residue.

In some embodiments, the covalent bond between the exogenous compound and the lysine residue is a reversible covalent bond, wherein the lysine residue is selected from K6, K9, K20, K24, K33, K34, K56, K65, K88, K89, K105, K129, K142, K178, K237, K242, K250, K273, K278, and K291. In some embodiments, the covalent bond between the exogenous compound and the lysine residue is a reversible covalent bond selected from a single bond, a double bound, and a triple bond, wherein the lysine residue is selected from K6, K9, K20, K24, K33, K34, K56, K65, K88, K89, K105, K129, K142, K178, K237, K242, K250, K273, K278, and K291. In some embodiments, the covalent bond between the exogenous compound and the lysine residue is a reversible covalent single bond, wherein the lysine residue is selected from K6, K9, K20, K24, K33, K34, K56, K65, K88, K89, K105, K129, K142, K178, K237, K242, K250, K273, K278, and K291. In some embodiments, the covalent bond between the exogenous compound and the lysine residue is a reversible covalent double bond, wherein the lysine residue is selected from K6, K9, K20, K24, K33, K34, K56, K65, K88, K89, K105, K129, K142, K178, K237, K242, K250, K273, K278, and K291.

In some embodiments, the covalent bond between the exogenous compound and the lysine residue is a reversible covalent bond, wherein the lysine residue is selected from K20, K33, K89, K129, K178, and K278. In some embodiments, the covalent bond between the exogenous compound and the lysine residue is a reversible covalent bond, wherein the lysine residue is selected from K20, K33, K129, K178, and K278. In some embodiments, the covalent bond between the exogenous compound and the lysine residue is a reversible covalent bond, wherein the lysine residue is K89. In some embodiments, the covalent bond between the exogenous compound and the lysine residue is a reversible covalent bond selected from a single bond, a double bound, and a triple bond, wherein the lysine residue is K89. In some embodiments, the covalent bond between the exogenous compound and the lysine residue is a reversible covalent single bond, wherein the lysine residue is K89. In some embodiments, the covalent bond between the exogenous compound and the lysine residue is a reversible covalent double bond, wherein the lysine residue is K89.

In some embodiments, the reversible covalent bond in the in vivo CDK2 protein comprises a carbon-nitrogen interaction. In some embodiments, the carbon-nitrogen interaction is selected form a carbon-nitrogen single bond and a carbon-nitrogen double bond. In some embodiments, the carbon-nitrogen interaction is a carbon-nitrogen single bond. In some embodiments, the carbon-nitrogen interaction is a carbon-nitrogen double bond.

In some embodiments, the CDK2 protein is covalently bound with the exogenous compound, wherein the exogenous compound is bound at only one residue of the CDK2 protein. In some embodiments, the CDK2 protein is covalently bond with the exogenous compound via one covalent bond. In some embodiments, the CDK2 protein is covalently bound with the exogenous compound, wherein the exogenous compound is bound at one lysine residue. In some embodiments, the CDK2 protein has a single covalent bond between a lysine residue and the exogenous compound. In some embodiments, the CDK2 protein has a single covalent bond between K89 and the exogenous compound. In some embodiments, the CDK2 protein has a single covalent bond between K20 and the exogenous compound. In some embodiments, the CDK2 protein has a single covalent bond between K33 and the exogenous compound. In some embodiments, the CDK2 protein has a single covalent bond between K129 and the exogenous compound. In some embodiments, the CDK2 protein has a single covalent bond between K178 and the exogenous compound. In some embodiments, the CDK2 protein has a single covalent bond between K278 and the exogenous compound.

In some embodiments, the exogenous compound has reduced engagement at other lysine residues when covalently bound at a lysine residue selected from K6, K9, K20, K24, K33, K34, K56, K65, K88, K89, K105, K129, K142, K178, K237, K242, K250, K273, K278, and K291. In some embodiments, the exogenous compound is in contact with one lysine residue selected from K6, K9, K20, K24, K33, K34, K56, K65, K88, K89, K105, K129, K142, K178, K237, K242, K250, K273, K278, and K291, and has reduced engagement at the remaining lysine residues. In some embodiments, the exogenous compound is in contact with K89, and has reduced engagement at K6, K9, K20, K24, K33, K34, K56, K65, K88, K105, K129, K142, K178, K237, K242, K250, K273, K278, and K291. In some embodiments, the exogenous compound is in contact with K20, and has reduced engagement at K6, K9, K24, K33, K34, K56, K65, K88, K89, K105, K129, K142, K178, K237, K242, K250, K273, K278, and K291. In some embodiments, the exogenous compound is in contact with K33, and has reduced engagement at K6, K9, K20, K24, K34, K56, K65, K88, K89, K105, K129, K142, K178, K237, K242, K250, K273, K278, and K291. In some embodiments, the exogenous compound is in contact with K129, and has reduced engagement at K6, K9, K20, K24, K33, K34, K56, K65, K88, K89, K105, K142, K178, K237, K242, K250, K273, K278, and K291. In some embodiments, the exogenous compound is in contact with K178, and has reduced engagement at K6, K9, K20, K24, K33, K34, K56, K65, K88, K89, K105, K129, K142, K237, K242, K250, K273, K278, and K291. In some embodiments, the exogenous compound is in contact with K278, and has reduced engagement at K6, K9, K20, K24, K33, K34, K56, K65, K88, K89, K105, K129, K142, K178, K237, K242, K250, K273, K278, and K291.

In some embodiments, the CDK2 protein has a single lysine residue covalently bound with the exogenous compound. In some embodiments, the CDK2 protein has a single lysine residue covalently bound with the exogenous compound, wherein the single lysine residue is selected from K6, K9, K20, K24, K33, K34, K56, K65, K88, K89, K105, K129, K142, K178, K237, K242, K250, K273, K278, and K291. In some embodiments, the CDK2 protein has a single lysine residue covalently bound with the exogenous compound, wherein the single lysine residue is K89. In some embodiments, the CDK2 protein has a single lysine residue covalently bound with the exogenous compound, wherein the single lysine residue is K20. In some embodiments, the CDK2 protein has a single lysine residue covalently bound with the exogenous compound, wherein the single lysine residue is K33. In some embodiments, the CDK2 protein has a single lysine residue covalently bound with the exogenous compound, wherein the single lysine residue is K129. In some embodiments, the CDK2 protein has a single lysine residue covalently bound with the exogenous compound, wherein the single lysine residue is K178. In some embodiments, the CDK2 protein has a single lysine residue covalently bound with the exogenous compound, wherein the single lysine residue is K278.

In some embodiments, the CDK2 protein is in vivo. In some embodiments, the CDK2 protein is in vitro. In some embodiments, the CDK2 protein is ex vivo. In some embodiments, the CDK2 protein is an in vivo engineered protein.

In some embodiments, the CDK2 protein is an in vivo engineered CDK2 protein, wherein the in vivo engineered CDK2 protein is generated by contacting the CDK2 protein in vivo with the exogenous compound. In some embodiments, the CDK2 protein is a mammalian in vivo engineered CDK2 protein, wherein the in vivo engineered CDK2 protein is generated by contacting the CDK2 protein in vivo with the exogenous compound. In some embodiments, the CDK2 protein is a human in vivo engineered CDK2 protein, wherein the in vivo engineered CDK2 protein is generated by contacting the CDK2 protein in vivo with the exogenous compound.

In some embodiments, the reversible covalent bond in the in vivo CDK2 protein is between a carbon atom and a nitrogen atom. In some embodiments, the reversible covalent bond in the in vivo CDK2 protein is between a carbon atom of the exogenous compound and a nitrogen atom of the lysine residue. In some embodiments, the reversible covalent bond in the in vivo CDK2 protein is between a carbon atom of the exogenous compound and a nitrogen atom of the lysine residue selected from K6, K9, K20, K24, K33, K34, K56, K65, K88, K89, K105, K129, K142, K178, K237, K242, K250, K273, K278, and K291. In some embodiments, the reversible covalent bond in the in vivo CDK2 protein is between a carbon atom of the exogenous compound and a nitrogen atom of the K89 lysine residue. In some embodiments, the reversible covalent bond in the in vivo CDK2 protein is between a carbon atom of the exogenous compound and a nitrogen atom of the K20 lysine residue. In some embodiments, the reversible covalent bond in the in vivo CDK2 protein is between a carbon atom of the exogenous compound and a nitrogen atom of the K33 lysine residue. In some embodiments, the reversible covalent bond in the in vivo CDK2 protein is between a carbon atom of the exogenous compound and a nitrogen atom of the K129 lysine residue. In some embodiments, the reversible covalent bond in the in vivo CDK2 protein is between a carbon atom of the exogenous compound and a nitrogen atom of the K178 lysine residue. In some embodiments, the reversible covalent bond in the in vivo CDK2 protein is between a carbon atom of the exogenous compound and a nitrogen atom of the K278 lysine residue.

In some embodiments, the reversible covalent bond in the in vivo CDK2 protein is a carbon-nitrogen double bond. In some embodiments, the reversible covalent bond in the in vivo CDK2 protein is a carbon-nitrogen double bond between the exogenous compound and the lysine residue. In some embodiments, the carbon-nitrogen double bond is between the exogenous compound and the lysine residue, wherein the lysine residue is selected from K6, K9, K20, K24, K33, K34, K56, K65, K88, K89, K105, K129, K142, K178, K237, K242, K250, K273, K278, and K291. In some embodiments, the carbon-nitrogen double bond is between the exogenous compound and the K89 lysine residue. In some embodiments, the carbon-nitrogen double bond is between the exogenous compound and the K20 lysine residue. In some embodiments, the carbon-nitrogen double bond is between the exogenous compound and the K33 lysine residue. In some embodiments, the carbon-nitrogen double bond is between the exogenous compound and the K129 lysine residue. In some embodiments, the carbon-nitrogen double bond is between the exogenous compound and the K178 lysine residue. In some embodiments, the carbon-nitrogen double bond is between the exogenous compound and the K278 lysine residue.

In some embodiments, the carbon-nitrogen double bond is an imine bond. In some embodiments, the imine bond is between a carbon atom of the exogenous compound and a nitrogen atom of the lysine residue. In some embodiments, the imine bond is between the exogenous compound and the lysine residue, wherein the lysine residue is selected from K6, K9, K20, K24, K33, K34, K56, K65, K88, K89, K105, K129, K142, K178, K237, K242, K250, K273, K278, and K291. In some embodiments, the imine bond is between the exogenous compound and the K89 lysine residue. In some embodiments, the imine bond is between the exogenous compound and the K20 lysine residue. In some embodiments, the imine bond is between the exogenous compound and the K33 lysine residue. In some embodiments, the imine bond is between the exogenous compound and the K129 lysine residue. In some embodiments, the imine bond is between the exogenous compound and the K178 lysine residue. In some embodiments, the imine bond is between the exogenous compound and the K278 lysine residue.

In some embodiments, the exogenous compound comprises a functional group. In some embodiments, the exogenous compound comprises an aldehyde functional group. In some embodiments, the reversible covalent bond is between the aldehyde functional group and the lysine residue. In some embodiments, the reversible covalent bond is between the aldehyde functional group and the lysine residue selected from K6, K9, K20, K24, K33, K34, K56, K65, K88, K89, K105, K129, K142, K178, K237, K242, K250, K273, K278, and K291. In some embodiments, the reversible covalent bond is between the aldehyde functional group and the K89 lysine residue. In some embodiments, the reversible covalent bond is between the aldehyde functional group and the K20 lysine residue. In some embodiments, the reversible covalent bond is between the aldehyde functional group and the K33 lysine residue. In some embodiments, the reversible covalent bond is between the aldehyde functional group and the K129 lysine residue. In some embodiments, the reversible covalent bond is between the aldehyde functional group and the K178 lysine residue. In some embodiments, the reversible covalent bond is between the aldehyde functional group and the K278 lysine residue.

In some embodiments, the carbon-nitrogen bond is a reversible bond that results from a reversible reaction. In some embodiments, the carbon-nitrogen bond is a reversible bond that results from a reversible reaction between the exogenous compound and a lysine residue. In some embodiments, the carbon-nitrogen bond is a reversible bond that results from a reversible reaction between the exogenous compound and a lysine residue selected from K6, K9, K20, K24, K33, K34, K56, K65, K88, K89, K105, K129, K142, K178, K237, K242, K250, K273, K278, and K291. In some embodiments, the carbon-nitrogen bond is a reversible bond that results from a reversible reaction between the exogenous compound and the K89 lysine residue. In some embodiments, the carbon-nitrogen bond is a reversible bond that results from a reversible reaction between the exogenous compound and the K20 lysine residue. In some embodiments, the carbon-nitrogen bond is a reversible bond that results from a reversible reaction between the exogenous compound and the K33 lysine residue. In some embodiments, the carbon-nitrogen bond is a reversible bond that results from a reversible reaction between the exogenous compound and the K129 lysine residue. In some embodiments, the carbon-nitrogen bond is a reversible bond that results from a reversible reaction between the exogenous compound and the K178 lysine residue. In some embodiments, the carbon-nitrogen bond is a reversible bond that results from a reversible reaction between the exogenous compound and the K278 lysine residue.

In some embodiments, the carbon-nitrogen double bond results from a reversible reaction between the amine functional group of a lysine residue and an aldehyde functional group on the exogenous compound. In some embodiments, the carbon-nitrogen double bond results from a reversible reaction between the amine functional group of a lysine residue and an aldehyde functional group on the exogenous compound, wherein the lysine residue is selected from K6, K9, K20, K24, K33, K34, K56, K65, K88, K89, K105, K129, K142, K178, K237, K242, K250, K273, K278, and K291. In some embodiments, the carbon-nitrogen double bond results from a reversible reaction between the amine functional group of K89 and an aldehyde functional group on the exogenous compound. In some embodiments, the carbon-nitrogen double bond results from a reversible reaction between the amine functional group of K20 and an aldehyde functional group on the exogenous compound. In some embodiments, the carbon-nitrogen double bond results from a reversible reaction between the amine functional group of K33 and an aldehyde functional group on the exogenous compound. In some embodiments, the carbon-nitrogen double bond results from a reversible reaction between the amine functional group of K129 and an aldehyde functional group on the exogenous compound. In some embodiments, the carbon-nitrogen double bond results from a reversible reaction between the amine functional group of K178 and an aldehyde functional group on the exogenous compound. In some embodiments, the carbon-nitrogen double bond results from a reversible reaction between the amine functional group of K278 and an aldehyde functional group on the exogenous compound.

In some embodiments, the aldehyde functional group of the exogenous compound is an aromatic aldehyde. In some embodiments, the carbon-nitrogen double bond results from a reversible reaction between the amine functional group of a lysine residue and the aromatic aldehyde group on the exogenous compound. In some embodiments, the carbon-nitrogen double bond results from a reversible reaction between the amine functional group of a lysine residue and the aromatic aldehyde group on the exogenous compound, wherein the lysine residue is selected from K6, K9, K20, K24, K33, K34, K56, K65, K88, K89, K105, K129, K142, K178, K237, K242, K250, K273, K278, and K291. In some embodiments, the carbon-nitrogen double bond results from a reversible reaction between the amine functional group of K89 and the aromatic aldehyde group on the exogenous compound. In some embodiments, the carbon-nitrogen double bond results from a reversible reaction between the amine functional group of K20 and the aromatic aldehyde group on the exogenous compound. In some embodiments, the carbon-nitrogen double bond results from a reversible reaction between the amine functional group of K33 and the aromatic aldehyde group on the exogenous compound. In some embodiments, the carbon-nitrogen double bond results from a reversible reaction between the amine functional group of K129 and the aromatic aldehyde group on the exogenous compound. In some embodiments, the carbon-nitrogen double bond results from a reversible reaction between the amine functional group of K178 and the aromatic aldehyde group on the exogenous compound. In some embodiments, the carbon-nitrogen double bond results from a reversible reaction between the amine functional group of K278 and the aromatic aldehyde group on the exogenous compound.

In some embodiments, the CDK2 inhibitor is a compound or salt as disclosed herein. In some embodiments, the CDK2 inhibitor is selected from a compound or salt of Formula (I), (II), (II-A), (II-B), (II-C), (II-D)), (II-E), (II-F), (III), (IV), (IV-A), (IV-B), (IV-C), or (V). In some embodiments, the CDK2 inhibitor is selected from a compound or salt in Table 1.

In some embodiments, the exogenous compound is a compound or salt as disclosed herein. In some embodiments, the exogenous compound is selected from a compound or salt of Formula (I), (II), (II-A), (II-B), (II-C), (II-D)), (II-E), (II-F), (III), (IV), (IV-A), (IV-B), (IV-C), or (V). In some embodiments, the exogenous compound is selected from a compound or salt in Table 1.

In some embodiments, the CDK2 inhibitor is a compound or salt as disclosed herein. In some embodiments, the CDK2 inhibitor is selected from a compound or salt of Formula (II). In some embodiments, the CDK2 inhibitor is selected from a compound or salt in Table 1A.

In some embodiments, the exogenous compound is a compound or salt as disclosed herein. In some embodiments, the exogenous compound is selected from a compound or salt of Formula (II). In some embodiments, the exogenous compound is selected from a compound or salt in Table 1A.

In certain aspects the present disclosure provides an in vivo engineered CDK2 protein comprising a non-naturally occurring reversible covalent modification at a lysine residue, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered CDK2 protein is a human in vivo engineered CDK2 protein.

In another aspect, the present disclosure provides an in vivo engineered CDK2 protein comprising a non-naturally occurring reversible covalent modification at a lysine residue selected from K6, K9, K20, K24, K33, K34, K56, K65, K88, K89, K105, K129, K142, K178, K237, K242, K250, K273, K278, and K291, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered CDK2 protein is a human in vivo engineered CDK2 protein.

In another aspect, the present disclosure provides an in vivo engineered CDK2 protein comprising a non-naturally occurring reversible covalent modification at a lysine residue selected from K20, K33, K89, K129, K178, and K278, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered CDK2 protein is a human in vivo engineered CDK2 protein.

In another aspect, the present disclosure provides an in vivo engineered CDK2 protein comprising a non-naturally occurring reversible covalent modification at a K89 lysine residue, the reversible covalent modification being generated from an in vivo nucleophilic reaction between an exogenous aromatic aldehyde and the K89 lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered CDK2 protein is a human in vivo engineered CDK2 protein.

In another aspect, the present disclosure provides an in vivo engineered CDK2 protein comprising a non-naturally occurring reversible covalent modification at a lysine residue selected from K6, K9, K24, K34, K56, K65, K88, K105, K142, K237, K242, K250, K273, and K291, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered CDK2 protein is a human in vivo engineered CDK2 protein.

In another aspect, the present disclosure provides an in vivo engineered CDK2 protein comprising a non-naturally occurring reversible covalent modification at a lysine residue selected from K20, K33, K129, K178, and K278, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered CDK2 protein is a human in vivo engineered CDK2 protein.

In certain aspects the present disclosure provides a human in vivo engineered CDK2 protein comprising a non-naturally occurring reversible covalent modification at a lysine residue selected K6, K9, K20, K24, K33, K34, K56, K65, K88, K89, K105, K129, K142, K178, K237, K242, K250, K273, K278, and K291, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect the present disclosure provides a human in vivo engineered CDK2 protein comprising a non-naturally occurring reversible covalent modification at a lysine residue selected from K20, K33, K89, K129, K178, and K278, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered CDK2 protein comprising a non-naturally occurring reversible covalent modification at a K89 lysine residue, the reversible covalent modification being generated from an in vivo nucleophilic reaction between an exogenous aromatic aldehyde and the K89 lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered CDK2 protein comprising a non-naturally occurring reversible covalent modification at a lysine residue selected from K6, K9, K24, K34, K56, K65, K88, K105, K142, K237, K242, K250, K273, and K291, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered CDK2 protein comprising a non-naturally occurring reversible covalent modification at a lysine residue selected from K20, K33, K89, K129, K178, and K278, the reversible covalent modification being generated from an in vivo nucleophilic reaction between an exogenous aromatic aldehyde and the lysine residue of CDK2, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

### Method of Modifying CDK2 Proteins

In some aspects, the present disclosure provides a method of modifying an CDK2 protein as disclosed herein. In some embodiments, the method of covalently modifying an CDK2 protein, comprises contacting the CDK2 protein with an exogenous compound, wherein the exogenous compound comprises a reversible electrophilic moiety thereby forming a reversible covalent CDK2 adduct. In some embodiments, the contacting is in vitro or in vivo. In some embodiments, the contacting is in vitro. In some embodiments, the contacting is in vivo. In some embodiments, the CDK2 protein is wild type CDK2 or a mutated CDK2. In some embodiments, the wild type CDK2 protein is wild type. In some embodiments, the exogenous compound is an CDK2 inhibitor. In some embodiments, the reversible covalent moiety on the CDK2 inhibitor is an aromatic aldehyde. In some embodiments, the reversible covalent CDK2 adduct is formed between the reversible covalent moiety and a lysine reside of the CDK2 protein. In some embodiments, the reversible covalent CDK2 adduct is formed between the aromatic aldehyde and the lysine residue of the CDK2 protein. In some embodiments, reversible covalent CDK2 adduct is formed between the reversible covalent moiety and a lysine residue of the CDK2 protein selected from K6, K9, K20, K24, K33, K34, K56, K65, K88, K89, K105, K129, K142, K178, K237, K242, K250, K273, K278, and K291. In some embodiments, reversible covalent CDK2 adduct is formed between the aromatic aldehyde and a lysine residue of the CDK2 protein selected from K6, K9, K20, K24, K33, K34, K56, K65, K88, K89, K105, K129, K142, K178, K237, K242, K250, K273, K278, and K291. In some embodiments, reversible covalent CDK2 adduct is formed between the aromatic aldehyde and the K89 lysine residue of the CDK2 protein.

In another aspect, the method of covalently modifying an CDK2 protein, comprises contacting the CDK2 protein with an exogenous CDK2 modulator, wherein the CDK2 modulator comprises a reversible electrophilic moiety thereby forming a reversible covalent CDK2 adduct. In some embodiments, the contacting is in vitro or in vivo. In some embodiments, the contacting is in vitro. In some embodiments, the contacting is in vivo. In some embodiments, the CDK2 protein is wild type CDK2 or a mutated CDK2. In some embodiments, the wild type CDK2 protein is wild type. In some embodiments, the reversible covalent moiety on the CDK2 modulator is an aromatic aldehyde. In some embodiments, the reversible covalent CDK2 adduct is formed between the reversible covalent moiety and a lysine reside of the CDK2 protein. In some embodiments, the reversible covalent CDK2 adduct is formed between the aromatic aldehyde and the lysine residue of the CDK2 protein. In some embodiments, reversible covalent CDK2 adduct is formed between the reversible covalent moiety and a lysine residue of the CDK2 protein selected from K6, K9, K20, K24, K33, K34, K56, K65, K88, K89, K105, K129, K142, K178, K237, K242, K250, K273, K278, and K291. In some embodiments, reversible covalent CDK2 adduct is formed between the aromatic aldehyde and a lysine residue of the CDK2 protein selected from K6, K9, K20, K24, K33, K34, K56, K65, K88, K89, K105, K129, K142, K178, K237, K242, K250, K273, K278, and K291. In some embodiments, reversible covalent CDK2 adduct is formed between the aromatic aldehyde and the K89 lysine residue of the CDK2 protein. In some embodiments, the exogenous CDK2 modulator is an CDK2 inhibitor.

In certain aspects the present disclosure provides a method of attenuating CDK2 activity. In some embodiments, the method of covalently modifying an CDK2 protein, comprises contacting the CDK2 protein with an exogenous CDK2 inhibitor, wherein the CDK2 modulator comprises a reversible electrophilic moiety thereby forming a reversible covalent CDK2 adduct. In some embodiments, the contacting is in vitro or in vivo. In some embodiments, the contacting is in vitro. In some embodiments, the contacting is in vivo. In some embodiments, the CDK2 protein is wild type CDK2 or a mutated CDK2. In some embodiments, the wild type CDK2 protein is wild type. In some embodiments, the reversible covalent moiety on the CDK2 inhibitor is an aromatic aldehyde. In some embodiments, the reversible covalent CDK2 adduct is formed between the reversible covalent moiety and a lysine reside of the CDK2 protein. In some embodiments, the reversible covalent CDK2 adduct is formed between the aromatic aldehyde and the lysine residue of the CDK2 protein. In some embodiments, reversible covalent CDK2 adduct is formed between the reversible covalent moiety and a lysine residue of the CDK2 protein selected from K6, K9, K20, K24, K33, K34, K56, K65, K88, K89, K105, K129, K142, K178, K237, K242, K250, K273, K278, and K291. In some embodiments, reversible covalent CDK2 adduct is formed between the aromatic aldehyde and a lysine residue of the CDK2 protein selected from K6, K9, K20, K24, K33, K34, K56, K65, K88, K89, K105, K129, K142, K178, K237, K242, K250, K273, K278, and K291. In some embodiments, reversible covalent CDK2 adduct is formed between the aromatic aldehyde and the K89 lysine residue of the CDK2 protein.

In some aspects, the method of attenuating CDK2 activity, comprises contacting CDK2 protein with an exogenous compound, wherein the exogenous compound comprises a reversible electrophilic moiety. In some embodiments, the CDK2 protein is wild type CDK2 or a mutated CDK2. In some embodiments, the wild type CDK2 protein is wild type. In some embodiments, the contacting is in vitro or in vivo. In some embodiments, the contacting is in vitro.

In some embodiments, following the contacting, the CDK2 activity is attenuated by 50% to 95% relative to a control in the absence of the exogenous compound. In some embodiments, following the contacting, the CDK2 activity is attenuated by 75% to 95% relative to a control in the absence of the exogenous compound. In some embodiments, following the contacting, the CDK2 activity is attenuated by 50%, 55%, 60%, 65%, 70%, 75%, 80%, 85%, 90%, 95%, or more relative to a control in the absence of the exogenous compound. In some embodiments, following the contacting, the CDK2 activity is attenuated by 50% or more relative to a control in the absence of the exogenous compound. In some embodiments, following the contacting, the CDK2 activity is attenuated by 70% or more relative to a control in the absence of the exogenous compound.

In some embodiments, following the contacting, the CDK2 activity is attenuated by about 50% to about 95% relative to a control in the absence of the exogenous compound. In some embodiments, following the contacting, the CDK2 activity is attenuated by about 75% to about 95% relative to a control in the absence of the exogenous compound. In some embodiments, following the contacting, the CDK2 activity is attenuated by about 50%, about 55%, about 60%, about 65%, about 70%, about 75%, about 80%, about 85%, about 90%, about 95%, or more relative to a control in the absence of the exogenous compound. In some embodiments, following the contacting, the CDK2 activity is attenuated by about 50% or more relative to a control in the absence of the exogenous compound. In some embodiments, following the contacting, the CDK2 activity is attenuated by about 70% or more relative to a control in the absence of the exogenous compound.

In some embodiments, following the contacting, the CDK2 activity is attenuated by at least 50% to at least 95% relative to a control in the absence of the exogenous compound. In some embodiments, following the contacting, the CDK2 activity is attenuated by at least 75% to at least 95% relative to a control in the absence of the exogenous compound. In some embodiments, following the contacting, the CDK2 activity is attenuated by at least 50%, at least 55%, at least 60%, at least 65%, at least 70%, at least 75%, at least 80%, at least 85%, at least 90%, or at least 95% relative to a control in the absence of the exogenous compound. In some embodiments, following the contacting, the CDK2 activity is attenuated by at least 50% relative to a control in the absence of the exogenous compound. In some embodiments, following the contacting, the CDK2 activity is attenuated by at least 70% relative to a control in the absence of the exogenous compound.

In some embodiments, following the contacting, the CDK2 activity is attenuated by at most 50% to at most 95% relative to a control in the absence of the exogenous compound. In some embodiments, following the contacting, the CDK2 activity is attenuated by at most 75% to at most 95% relative to a control in the absence of the exogenous compound. In some embodiments, following the contacting, the CDK2 activity is attenuated by at most 50% relative to a control in the absence of the exogenous compound. In some embodiments, following the contacting, the CDK2 activity is attenuated by at most 70% relative to a control in the absence of the exogenous compound. In some embodiments, following the contacting, the CDK2 activity is attenuated by at most 50%, at most 55%, at most 60%, at most 65%, at most 70%, at most 75%, at most 80%, at most 85%, at most 90%, or at most 95% relative to a control in the absence of the exogenous compound.

In another aspect, the present disclosure provides a method of attenuating CDK2 activity, comprising contacting CDK2 protein with an CDK2 inhibitor, wherein the CDK2 inhibitor comprises a reversible electrophilic moiety. In some embodiments, the CDK2 protein is wild type CDK2 or a mutated CDK2. In some embodiments, the wild type CDK2 protein is wild type. In some embodiments, the contacting is in vitro or in vivo. In some embodiments, the contacting is in vitro.

In some embodiments, following the contacting, the CDK2 activity is attenuated by 50% to 95% relative to a control in the absence of the exogenous CDK2 inhibitor. In some embodiments, following the contacting, the CDK2 activity is attenuated by 75% to 95% relative to a control in the absence of the exogenous CDK2 inhibitor. In some embodiments, following the contacting, the CDK2 activity is attenuated by 50%, 55%, 60%, 65%, 70%, 75%, 80%, 85%, 90%, 95%, or more relative to a control in the absence of the exogenous CDK2 inhibitor. In some embodiments, following the contacting, the CDK2 activity is attenuated by 50% or more relative to a control in the absence of the exogenous CDK2 inhibitor. In some embodiments, following the contacting, the CDK2 activity is attenuated by 70% or more relative to a control in the absence of the exogenous CDK2 inhibitor.

In some embodiments, following the contacting, the CDK2 activity is attenuated by about 50% to about 95% relative to a control in the absence of the exogenous CDK2 inhibitor. In some embodiments, following the contacting, the CDK2 activity is attenuated by about 75% to about 95% relative to a control in the absence of the exogenous CDK2 inhibitor. In some embodiments, following the contacting, the CDK2 activity is attenuated by about 50%, about 55%, about 60%, about 65%, about 70%, about 75%, about 80%, about 85%, about 90%, about 95%, or more relative to a control in the absence of the exogenous CDK2 inhibitor. In some embodiments, following the contacting, the CDK2 activity is attenuated by about 50% or more relative to a control in the absence of the exogenous CDK2 inhibitor. In some embodiments, following the contacting, the CDK2 activity is attenuated by about 70% or more relative to a control in the absence of the exogenous CDK2 inhibitor.

In some embodiments, following the contacting, the CDK2 activity is attenuated by at least 50% to at least 95% relative to a control in the absence of the exogenous CDK2 inhibitor. In some embodiments, following the contacting, the CDK2 activity is attenuated by at least 75% to at least 95% relative to a control in the absence of the exogenous CDK2 inhibitor. In some embodiments, following the contacting, the CDK2 activity is attenuated by at least 50%, at least 55%, at least 60%, at least 65%, at least 70%, at least 75%, at least 80%, at least 85%, at least 90%, or at least 95% relative to a control in the absence of the exogenous CDK2 inhibitor. In some embodiments, following the contacting, the CDK2 activity is attenuated by at least 50% relative to a control in the absence of the exogenous CDK2 inhibitor. In some embodiments, following the contacting, the CDK2 activity is attenuated by at least 70% relative to a control in the absence of the exogenous CDK2 inhibitor.

In some embodiments, following the contacting, the CDK2 activity is attenuated by at most 50% to at most 95% relative to a control in the absence of the exogenous CDK2 inhibitor. In some embodiments, following the contacting, the CDK2 activity is attenuated by at most 75% to at most 95% relative to a control in the absence of the exogenous CDK2 inhibitor. In some embodiments, following the contacting, the CDK2 activity is attenuated by at most 50% relative to a control in the absence of the exogenous CDK2 inhibitor. In some embodiments, following the contacting, the CDK2 activity is attenuated by at most 70% relative to a control in the absence of the exogenous CDK2 inhibitor. In some embodiments, following the contacting, the CDK2 activity is attenuated by at most 50%, at most 55%, at most 60%, at most 65%, at most 70%, at most 75%, at most 80%, at most 85%, at most 90%, or at most 95% relative to a control in the absence of the exogenous CDK2 inhibitor.

### EXAMPLES

The invention now being generally described, it will be more readily understood by reference to the following examples which are included merely for purposes of illustration of certain aspects and embodiments of the present invention and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-38 show general and exemplary procedures for the preparation of the claimed CDK2 modulators. Examples 39 and 40 provide bioassay results for selected CDK2 modulators. Example 41 provides intact Mass Spectrometry and dissociation studies. Example 42 provides single crystal X-ray diffraction studies. Example 43 provides bioavailability data for imine prodrugs.

### Example 1: General Synthetic Schemes

### General Scheme A

The following Schemes describe illustrate various methods of preparation of compounds described herein. Schemes are exemplary and not exhaustive. It is understood that one skilled in the art may be able to synthesize described compounds by similar methods.

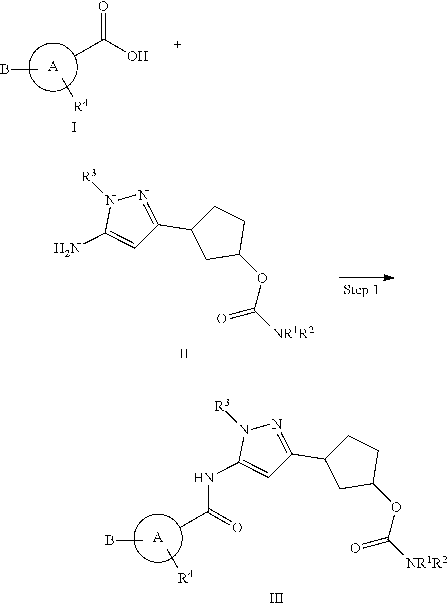

General Scheme A provides a general synthesis of amido-pyrazoles III. In Schemes disclosed herein, “A” is a 3- to 12-membered carbocycle or heterocycle possessing an amine reactive electrophile, “B”, such as an aldehyde that is optionally masked with a suitable protecting group. In Step 1, carboxylic acid I can readily undergo an amide coupling with amino-pyrazole II using an appropriate amide coupling reagent to provide III. In some cases, aryl carboxylic acid A I possessing the amine reactive electrophile “B” can be protected with a suitable protecting group such as an acetal with ethylene glycol, prior to amide coupling, additionally, in some cases the pyrazole nitrogen of amino-pyrazole II can be protected with a suitable protecting group such as a tert-butyl group prior to amide coupling. In either or a combination of these cases, the protected moieties can be deprotected with an appropriate acid such as trifluoroacetic acid or methanesulfonic acid to provide the desired amido-pyrazole.

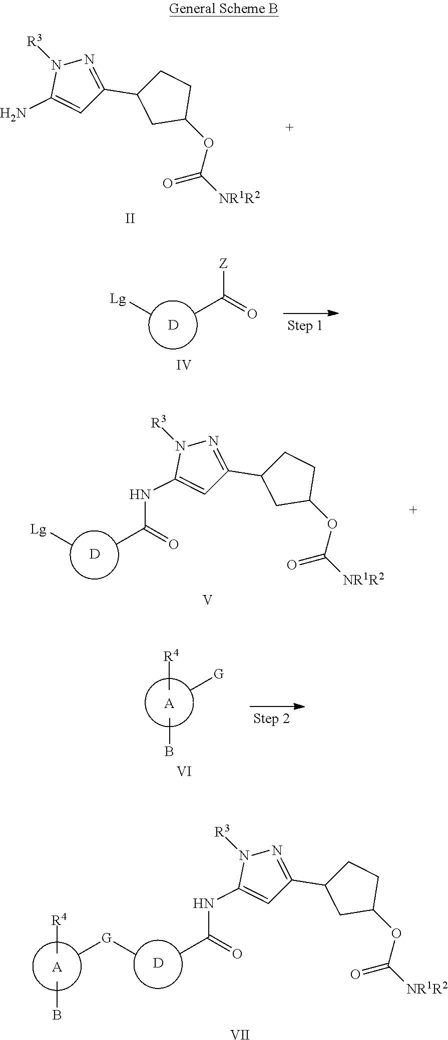

General Scheme B provides a general synthesis of amido-pyrazoles VII. In Schemes disclosed herein, “D” corresponds to a linker that is one to four atoms long or a 3- to 6-membered carbocycle or heterocycle consisting of C, O, N, or S. “Lg” corresponds to a leaving group that can participate in a nucleophilic substitution reaction (e.g: Cl, Br, I, OTs) or a hydroxyl group which can be displaced in a Mitsunobu reaction. “Z” corresponds to a group capable of reacting in an acyl substitution reaction or amide coupling reaction (e.g. Cl, Br, OH). “G” corresponds to a nucleophilic atom such as N, O, S, capable of reacting in a nucleophilic substitution or a Mitsunobu type reaction and is either directly attached to aryl-A or via a one to two carbon linker. In Step 1, carboxylic acid IV and amino-pyrazole II can readily undergo an amide coupling with an appropriate coupling reagent to provide amido-pyrazole intermediate V. In Step 2, intermediate amido-pyrazole V undergoes a nucleophilic substitution reaction with aryl-nucleophile VI to afford amido-pyrazole VII. In some cases, group A, VI, possessing the amine reactive electrophile “B” can be protected with a suitable protecting group such as an acetal with ethylene glycol, prior to nucleophilic substitution, additionally, in some cases the pyrazole nitrogen of amino-pyrazole II can be protected with a suitable protecting group such as a tert-butyl group prior to amide coupling. In either or a combination of these cases, the protected moieties can be deprotected with an appropriate acid such as trifluoroacetic acid or methanesulfonic acid to provide the desired amido-pyrazole.

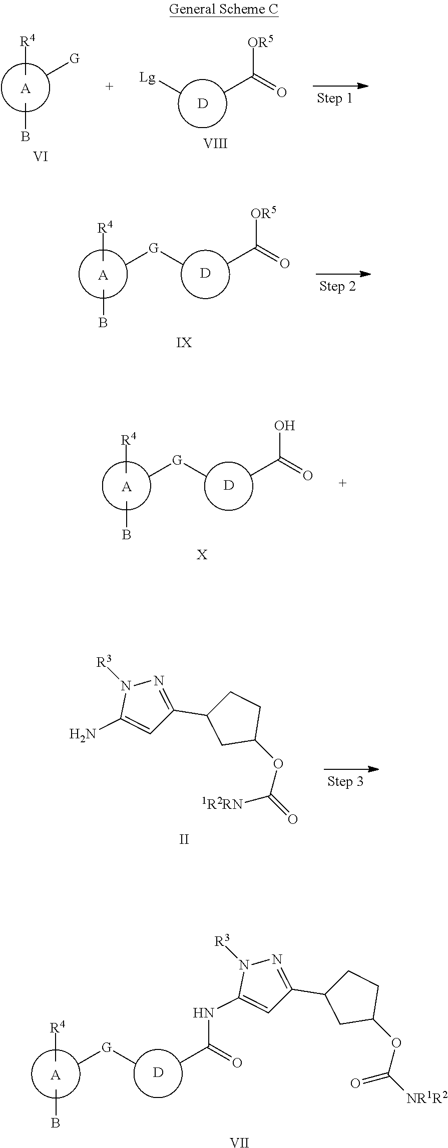

General Scheme C provides a general synthesis of amido-pyrazoles VII. In Step 1, leaving group possessing electrophile VIII, undergoes a nucleophilic substitution with aryl-nucleophile VI. In Step 2, protected carboxylic acid intermediate IX is treated with an appropriate base such as LiOH or NaOH to afford carboxylic acid X. In Step 3, carboxylic acid X is subjected to standard peptide coupling conditions with amino-pyrazine II to afford amido-pyrazole VII. In some cases, group A VI possessing the amine reactive electrophile “B” can be protected with a suitable protecting group such as an acetal with ethylene glycol, prior to nucleophilic substitution, additionally, in some cases the pyrazole nitrogen of amino-pyrazole II can be protected with a suitable protecting group such as a tert-butyl group prior to amide coupling. In either or a combination of these cases, the protected moieties can be deprotected with an appropriate acid such as trifluoroacetic acid or methanesulfonic acid to provide the desired amido-pyrazole.

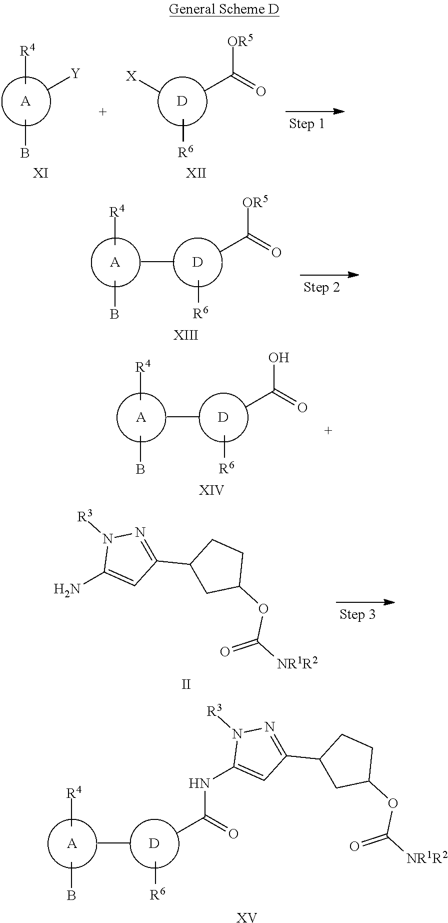

General Scheme D provides a general synthesis of amido-pyrazole XV. “X” corresponds to a halogen (X═Cl, Br, I) or psudohalogen capable of undergoing a Suzuki, Negishi, or Buchwald cross coupling reaction with an appropriate coupling partner. “Y” is a boronic ester, boronic acid, zincate, terminal alkyne, thiol, amine, or alcohol capable of undergoing a Suzuki, Negishi, Sonogashira cross coupling or Ullmann-type reaction with the appropriate coupling partner “X”. When step 1 is a Buchwald-type cross coupling or Ullmann condensation, “Y” can be absent and cross coupling proceeds through a heteroatom in aryl-A. For Scheme, aryl A can be an NH containing heterocycle such as a substituted pyrazole, or pyrrolidine. As an Scheme in Step 1, boronic ester XI can be cross coupled to aryl-halide XII, to form protected carboxylic acid intermediate XIII. In Step 2, protected ester XIII is treated with an appropriate base such as LiOH or NaOH to afford carboxylic acid intermediate XIV. In Step 3 carboxylic acid XIV can next be subjected to standard peptide coupling conditions with amine II to afford amido-pyrazole XV. In some cases, the coupling partner XI possessing the amine reactive electrophile “B” can be protected with a suitable protecting group such as an acetal with ethylene glycol, prior to cross coupling, additionally, in some cases the pyrazole nitrogen of amino-pyrazole II can be protected with a suitable protecting group such as a tert-butyl group prior to amide coupling. In either or a combination of these cases, the protected moieties can be deprotected with an appropriate acid such as trifluoroacetic acid or methanesulfonic acid to provide the desired amido-pyrazole.

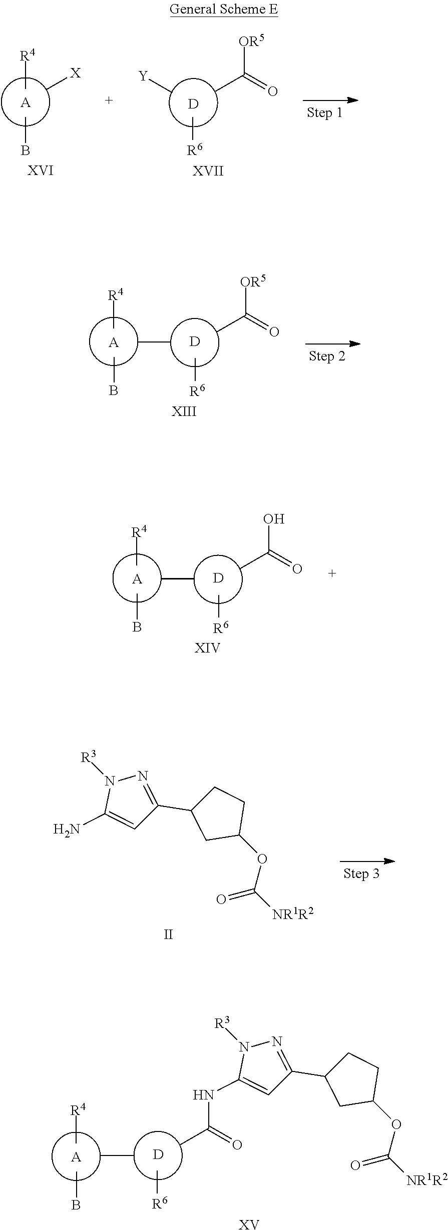

General Scheme E provides a general synthesis of amido-pyrazole XV. In Scheme E “X” corresponds to a halogen (X═Cl, Br, I) or psudohalogen capable of undergoing a Suzuki, Negishi, or Buchwald cross coupling reaction with an appropriate coupling partner. “Y” is a boronic ester, boronic acid, zincate, terminal alkyne, thiol, amine, or alcohol capable of undergoing a Suzuki, Negishi, Sonogashira cross coupling or Ullmann-type reaction with the appropriate coupling partner “X”. When step 1 is a Buchwald type or Ullmann-type cross coupling, ester XVII, “Y” can be absent and cross coupling proceeds through a heteroatom in linker-D. For Scheme, linker-D can be an NH containing heterocycle such as a substituted pyrazole, or pyrrolidine. As an Scheme n step 1, aryl-halide XVI, can be cross coupled to coupling partner, XVII, to form protected carboxylic acid intermediate XIII. In Step 2, protected carboxylic acid intermediate XIII is treated with an appropriate base such as LiOH or NaOH to afford carboxylic acid intermediate XIV. In Step 3, XIV can next be subjected to standard peptide coupling conditions with amine II to afford amido-pyrazole XV. In some cases, the coupling partner group A XIV possessing the amine reactive electrophile “B” can be protected with a suitable protecting group such as an acetal with ethylene glycol, prior to cross coupling, additionally, in some cases the pyrazole nitrogen of amino-pyrazole II can be protected with a suitable protecting group such as a tert-butyl group prior to amide coupling. In either or both cases, the protected moieties can be deprotected with an appropriate acid such as trifluoroacetic acid or methanesulfonic acid to provide the desired amido-pyrazole.

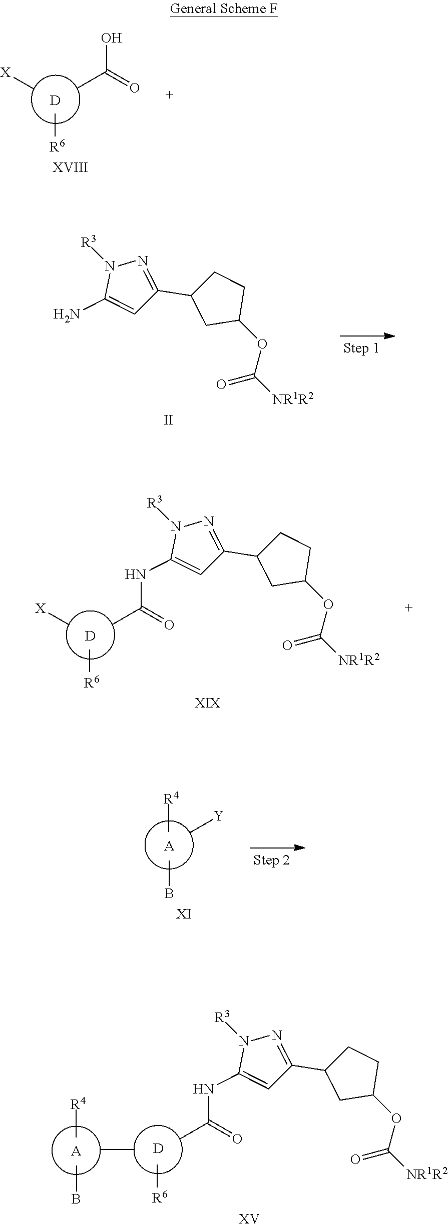

General Scheme F provides a general synthesis of amido-pyrazole. In Step 1, carboxylic acid XVIII, is subjected to standard peptide coupling conditions with amine II to afford intermediate amido-pyrazole XIX. In step 2, intermediate amido-pyrazole XIX can be cross coupled to aryl-boronic ester XI to afford amido-pyrazole XV. In some cases, the coupling partner XI possessing the amine reactive electrophile “B” can be protected with a suitable protecting group such as an acetal with ethylene glycol, prior to cross coupling, additionally, in some cases the pyrazole nitrogen of amino-pyrazole II can be protected with a suitable protecting group such as a tert-butyl group prior to amide coupling. In either or both cases, the protected moieties can be deprotected with an appropriate acid such as trifluoroacetic acid or methanesulfonic acid to provide the desired amido-pyrazole.

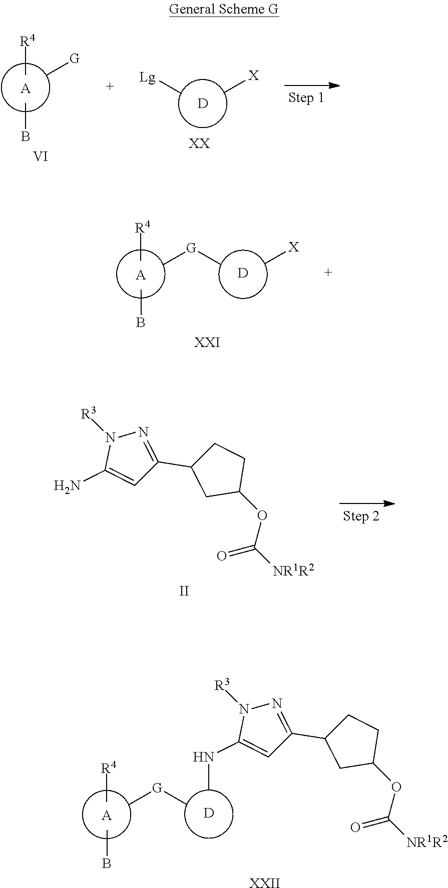

General Scheme G provides a general synthesis of amino-pyrazole XXII. In Step 1, electrophilic aryl-halide XX, undergoes a nucleophilic aromatic substitution with aryl-nucleophile VI to give halo-biaryl XXI. In Step 2, XXI can be cross coupled to amino-pyrazole II to afford amino-pyrazole XXII. In some cases, aryl group “A” VI possessing the amine reactive electrophile “B” can be protected with a suitable protecting group such as an acetal with ethylene glycol, prior to nucleophilic aromatic substitution, additionally, in some cases the pyrazole nitrogen of amino-pyrazole II can be protected with a suitable protecting group such as a tert-butyl group prior to amide coupling. In either or a combination of these cases, the protected moieties can be deprotected with an appropriate acid such as trifluoroacetic acid or methanesulfonic acid to provide the desired amido-pyrazole.

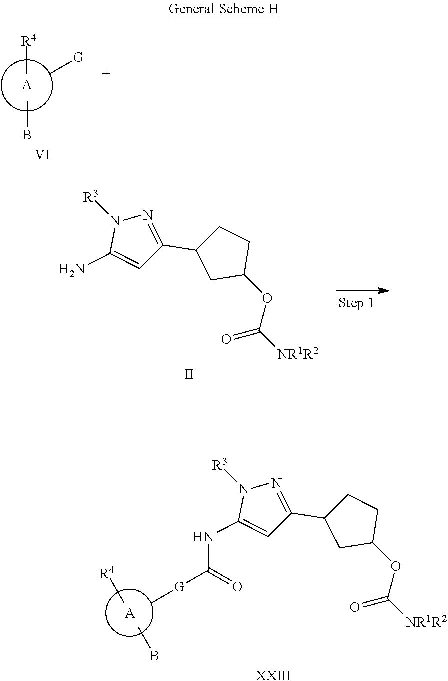

General Scheme H provides a general synthesis of carbamate-pyrazoles and urea-pyrazole XXIII. In Step 1, aryl-nucleophilic VI, and amino-pyrazole II are treated with phosgene or a phosgene equivalent to provide carbamate-pyrazole XXIII. In some cases, aryl-nucleophile, VI, possessing the amine reactive electrophile “B” can be protected with a suitable protecting group such as an acetal with ethylene glycol, prior to reaction with phosgene or a phosgene equivalent, additionally, in some cases the pyrazole nitrogen of amino-pyrazole II can be protected with a suitable protecting group such as a tert-butyl group prior to reaction with phosgene or a phosgene equivalent. In either or a combination of these cases, the protected moieties can be deprotected with an appropriate acid such as trifluoroacetic acid or methanesulfonic acid to provide the desired carbamate-pyrazoles or urea-pyrazole.

### Example 1A Procedures for Intermediates

### Example 1A.1: Synthesis of Intermediate A: 2-(2-formyl-3-hydroxy-5-methoxyphenoxy)acetic acid

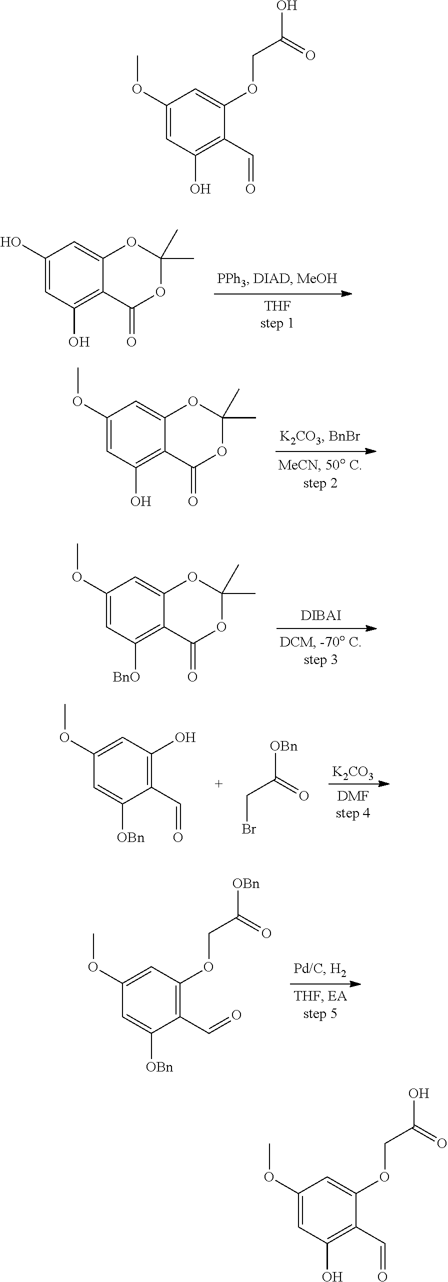

### Step 1: Synthesis of 5-hydroxy-7-methoxy-2,2-dimethyl-1,3-benzodioxin-4-one

5,7-dihydroxy-2,2-dimethyl-1,3-benzodioxin-4-one (1.0 g, 4.758 mmol), triphenylphosphine (1.6 g, 6.185 mmol), methanol (0.2 g, 6.185 mmol) and tetrahydrofuran (15 mL) were added to a 100 mL round-bottom flask and stirred until homogeneous, then treated with DIAD (1.3 g, 6.185 mmol) at 0° C. The resulting mixture was stirred under N₂ atmosphere at rt. for 2 h, quenched with H₂O and extracted with EA (100 mL×3). The combined organic layers were washed with brine, dried over anhydrous sodium sulfate, filtered, and concentrated under vacuum. The residue was purified by silica gel chromatography (0-40% PE/EA) to afford 5-hydroxy-7-methoxy-2,2-dimethyl-1,3-benzodioxin-4-one as a white solid (1.0 g, 93.74%). MS (ESI) mass calcd. for C₁₁H₁₂O₅, 224.06 m/z, found 223.10 m/z [M−H]⁺.

### Step 2: Synthesis of 5-(benzyloxy)-7-methoxy-2,2-dimethyl-1,3-benzodioxin-4-one

5-hydroxy-7-methoxy-2,2-dimethyl-1,3-benzodioxin-4-one (1.0 g, 4.460 mmol), a stir bar, K₂CO₃ (1.2 g, 8.920 mmol) and ACN (16 mL) were added to a 100 mL round-bottom flask and stirred until homogeneous, then treated with benzyl bromide (1.1 g, 6.690 mmol). The reaction mixture was stirred at 50° C. for 16 h, quenched with NH₄Cl and, extracted with EA (150 mL×3). The combined organic layers were washed with brine, dried over anhydrous sodium sulfate, filtered, and concentrated in vacuo. The residue was purified by silica gel chromatography (0-40% PE/EA) to afford 5-(benzyloxy)-7-methoxy-2,2-dimethyl-1,3-benzodioxin-4-one as a yellow oil (1.3 g, 92.73%). MS (ESI) mass calcd. for C₁₈H₁₈O₅, 314.12 m/z, found 315.10 m/z [M+H]⁻.

### Step 3: Synthesis of 2-(benzyloxy)-6-hydroxy-4-methoxybenzaldehyde

5-(benzyloxy)-7-methoxy-2,2-dimethyl-1,3-benzodioxin-4-one (1.3 g, 4.136 mmol), a stir bar, and DCM (20 mL) were added to a 100 mL round-bottom flask, placed under a N₂ atmosphere, and stirred until homogeneous. The resulting mixture was stirred at −70° C. for 0.5 h, followed by the dropwise addition of DIBAl (8.27 mL, 12.408 mmol, 1.5 M in toluene) −70° C. The resulting mixture was stirred at −70° C. for 3 h and quenched with MeOH (1 mL). The mixture was acidified with HCl aqueous (1 M) until pH=1 and left to stir at room temperature for 3 h. The reaction mixture was extracted with DCM (3×90 mL), the combined organic phase was washed with brine, dried with Na₂SO₄, filtered, and evaporated under reduced pressure. The residue was added to a combined solution of THF (5 mL) and HCl aqueous (2 M, 1 mL), and stirred at room temperature for 2 h. Then the mixture was diluted with H₂O, extracted with EA (3×90 mL), the combined organic phase was washed with brine, and dried with Na₂SO₄, filtered, and evaporated at reduced pressure. The residue was purified by silica gel chromatography (0-30% PE/EA) to afford 2-(benzyloxy)-6-hydroxy-4-methoxybenzaldehyde as a white solid (0.5 g, 46.81%). MS (ESI) mass calcd. for C₁₅H₁₄O₄, 258.09 m/z, found 259.00 [M+H]⁺.

### Step 4: Synthesis of benzyl 2-(3-(benzyloxy)-2-formyl-5-methoxyphenoxy)acetate

2-(benzyloxy)-6-hydroxy-4-methoxybenzaldehyde (50.0 g, 193.594 mmol), K₂CO₃ (66.89 g, 483.985 mmol), a stir bar, and DMF (600 mL) was added were added to a 2000 mL round-bottom flask and stirred until homogeneous, then treated with benzyl 2-bromoacetate (48.78 g, 212.953 mmol) at r.t. The reaction mixture was stirred at r.t. for 4 h. The resulting mixture was filtered, the filter cake was washed with EA (500 mL). The resulting mixture was diluted with water, and extracted with EA, the combined organic phase was washed with brine, dried with Na₂SO₄, filtered, and evaporated under reduced pressure. The residue was purified by silica gel chromatography (0-50% PE/EA) to afford benzyl 2-[3-(benzyloxy)-2-formyl-5-methoxyphenoxy]acetate (64 g, 81.34%) as a white solid. MS (ESI) mass calcd. for C₂₄H₂₂O₆, 406.14 m/z, found 407.20 [M+H]⁺.

### Step 5: Synthesis of Intermediate A, 2-(2-formyl-3-hydroxy-5-methoxyphenoxy)acetic acid

Benzyl 2-[3-(benzyloxy)-2-formyl-5-methoxyphenoxy]acetate (70.0 g, 172.230 mmol), THF (500 mL), a stir bar, and EA (500 mL) were added to a 3000 mL round-bottom flask and stirred until homogeneous, then treated with Pd/C (70 g, 10%). The resulting mixture was maintained under H₂ and stirred at r.t for 6 h, then filtered and concentrated to afford intermediate A (38.0 g, 97.55%, crude) as a yellow solid. The crude material was used without any further purification. MS (ESI) calcd. for C₁₀H₁₀O₆, 226.04 m/z, found: 227.20 [M+H]⁺.

### Example 1A.2: Synthesis of Intermediate B: 2-(3-(benzyloxy)-2-formyl-5-methoxyphenoxy)acetic acid

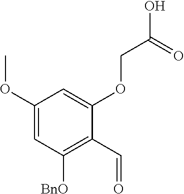 [SMILES: COc1cc(OCC(=O)O)c(C=O)c(OCc2ccccc2)c1]

### Step 1: Synthesis of 5-hydroxy-7-methoxy-2,2-dimethyl-1,3-benzodioxin-4-one

5,7-dihydroxy-2,2-dimethyl-1,3-benzodioxin-4-one (1 g, 4.758 mmol), triphenylphosphine (1.6 g, 6.185 mmol), methanol (0.2 g, 6.185 mmol) and tetrahydrofuran (15 mL) were added to a 100 mL round-bottom flask and stirred until homogeneous, then treated with DIAD (1.3 g, 6.185 mmol) at 0° C. The resulting mixture was stirred under N₂ atmosphere. The resulting mixture was stirred at rt. for 2 h followed by H₂O quenched and with extracted with EA (100 mL×3). The combined organic layers were washed with brine, dried over anhydrous sodium sulfate, filtered, and concentrated under vacuum. The residue was purified by silica gel chromatography (0-40% PE/EA) to afford 5-hydroxy-7-methoxy-2,2-dimethyl-1,3-benzodioxin-4-one as a white solid (1 g, 93.74%). MS (ESI) mass calcd. for C₁₁H₁₂O₅, 224.06 m/z, found 223.10 [M−H]−.

### Step 2: Synthesis of 5-(benzyloxy)-7-methoxy-2,2-dimethyl-1,3-benzodioxin-4-one

5-hydroxy-7-methoxy-2,2-dimethyl-1,3-benzodioxin-4-one (1 g, 4.460 mmol), a stir bar, K₂CO₃ (1.2 g, 8.920 mmol) and ACN (16 mL) were added to a 100 mL round-bottom flask and stirred until homogeneous, then treated with benzyl bromide (1.1 g, 6.690 mmol). The reaction mixture was stirred at 50° C. overnight and quenched with NH₄Cl and extracted with EA (150 mL×3). The combined organic layers were washed with brine, dried over anhydrous sodium sulfate, filtered, and concentrated under vacuum. The residue was purified by silica gel chromatography (0-40% PE/EA) to afford 5-(benzyloxy)-7-methoxy-2,2-dimethyl-1,3-benzodioxin-4-one as a yellow oil (1.3 g, 92.73%). MS (ESI) mass calcd. for C₁₈H₁₈O₅, 314.11 m/z, found 315.10 [M+H]⁺.

### Step 3: Synthesis of 2-(benzyloxy)-6-hydroxy-4-methoxybenzaldehyde

5-(benzyloxy)-7-methoxy-2,2-dimethyl-1,3-benzodioxin-4-one (1.3 g, 4.136 mmol), a stir bar, and DCM (20 mL) were added to a 100 mL round-bottom flask, placed under a N₂ atmosphere, and stirred until homogeneous. The resulting mixture was stirred at −70° C. for 0.5 h, followed by the dropwise addition of DIBAl (8.27 mL, 12.408 mmol, 1.5 M in toluene) −70° C. The resulting mixture was stirred at −70° C. for additional 3 h and quenched with MeOH (1 mL). The mixture was acidified with aqueous HCl (1 M) to pH=1 and left to stir at room temperature for 3 h. The reaction mixture was extracted with DCM (3×90 mL), the combined organic extracters were washed with brine, dried with Na₂SO₄, filtered, and evaporated under reduced pressure. The residue was added to a combined solution of THF (5 mL) and HCl aqueous (2 M, 1 mL), and stirred at room temperature for 2 h. The mixture was further diluted with H₂O, extracted with EA (3×90 mL), the combined organic phase were washed with brine, and over with Na₂SO₄, filtered, and evaporated under reduced pressure. The residue was purified by silica gel chromatography (0-30% PE/EA) to afford 2-(benzyloxy)-6-hydroxy-4-methoxybenzaldehyde as a white solid (0.5 g, 46.81%). MS (ESI) mass calcd. for C₁₅H₁₄O₄, 258.09 m/z, found 259.00 [M+H]⁺.

### Step 4: Synthesis of Ethyl 2-[3-(benzyloxy)-2-formyl-5-methoxyphenoxy]acetate

2-(benzyloxy)-6-hydroxy-4-methoxybenzaldehyde (400 mg, 1.549 mmol), a stir bar, K₂CO₃ (431 mg, 3.098 mmol) and acetone (10 mL) were added to a 100 mL round-bottom flask and stirred until homogeneous, then treated with ethyl bromoacetate (517 mg, 3.098 mmol) under N₂ atmosphere. The reaction mixture was stirred at rt. overnight, then diluted with water. The resulting mixture was extracted with EA (100 mL×3). The combined organic extracts were washed with H₂O (50 mL×3) and dried over anhydrous Na₂SO₄. After filtration, the filtrate was concentrated under reduced pressure. The residue was purified by silica gel column with PE/EA (0-60%) to afford ethyl 2-[3-(benzyloxy)-2-formyl-5-methoxyphenoxy]acetate (250 mg, 46.88%) as a white solid. MS (ESI) mass calcd. for C₁₉H₂₀O₆, 344.13 m/z, found 345.05 [M+H]⁺.

### Step 5: Synthesis of Intermediate B, 2-(3-(benzyloxy)-2-formyl-5-methoxyphenoxy)acetic acid

Ethyl 2-[3-(benzyloxy)-2-formyl-5-methoxyphenoxy]acetate (250 mg, 0.726 mmol), LiOH (24 mg, 1.016 mmol), tetrahydrofuran (5 mL) and, water (1 mL) were added to a 100 mL round-bottom flask and stirred until homogeneous. The resulting mixture was stirred at rt. for 1 h. The reaction mixture was acidified to pH=5 with HCl (2 M). The resulting mixture was extracted with EA (3×60 mL). The combined organic layers were washed with brine, dried over anhydrous sodium sulfate, filtered, and concentrated under vacuum to afford intermediate B as a white solid (230 g, 95.80%). MS (ESI) calcd. for C₁₇H₁₆O₆, 316.09 m/z, found 317.10 [M+H]⁺.

### Example 1A.3: Synthesis of Intermediate C: benzyl (5-((1S,3R)-3-(((4-nitrophenoxy)carbonyl)oxy)cyclopentyl)-1H-pyrazol-3-yl)carbamate

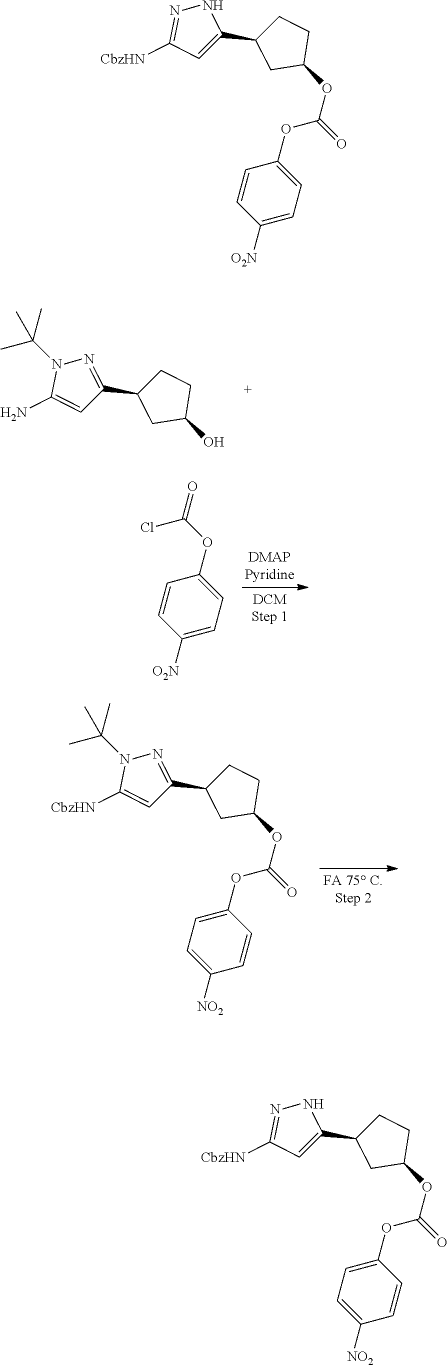 [SMILES: O=C(Nc1cc([C@H]2CC[C@@H](OC(=O)Oc3ccc([N+](=O)[O-])cc3)C2)[nH]n1)OCc1ccccc1 ; CC(C)(C)n1nc([C@H]2CC[C@@H](O)C2)cc1N ; O=C(Cl)Oc1ccc([N+](=O)[O-])cc1 ; CC(C)(C)n1nc([C@H]2CC[C@@H](OC(=O)Oc3ccc([N+](=O)[O-])cc3)C2)cc1NC(=O)OCc1ccccc1 ; O=C(Nc1cc([C@H]2CC[C@@H](OC(=O)Oc3ccc([N+](=O)[O-])cc3)C2)[nH]n1)OCc1ccccc1]

### Step 1: Synthesis of (1R,3S)-3-(5-{[(benzyloxy)carbonyl]amino}-1-tert-butylpyrazol-3-yl)cyclopentyl 4-nitrophenyl carbonate

To a stirred solution of benzyl N-{2-tert-butyl-5-[(1S,3R)-3-hydroxycyclopentyl]pyrazol-3-yl}carbamate (25.0 g, 69.939 mmol, 1.0 equiv) and 4-nitrophenyl chloroformate (16.35 g, 81.129 mmol, 1.16 equiv) in DCM (300 mL) was added pyridine (16.97 mL, 209.817 mmol, 3 equiv) and DMAP (0.85 g, 6.994 mmol, 0.10 equiv) at room temperature. The resulting mixture was stirred for 4 h, then diluted with water (100 mL) and extracted with DCM (3×500 mL). The combined organic layers were washed with brine (3×500 mL), dried over anhydrous Na₂SO₄. After filtration, the filtrate was concentrated under reduced pressure. This resulted in (1R,3S)-3-(5-{[(benzyloxy)carbonyl]amino}-1-tert-butylpyrazol-3-yl)cyclopentyl 4-nitrophenyl carbonate (35.0 g, 95.8%) as a yellow oil. MS (ESI) calcd. for C₂₇H₃₀N₄O₇, 522.21 m/z, found 523.15 [M+H]⁺.

### Step 2: Synthesis of Intermediate D, (1R,3S)-3-(5-{[(benzyloxy)carbonyl]amino}-1H-pyrazol-3-yl)cyclopentyl 4-nitrophenyl carbonate

A solution of (1R,3S)-3-(5-{[(benzyloxy)carbonyl]amino}-1-tert-butylpyrazol-3-yl)cyclopentyl 4-nitrophenyl carbonate (35.0 g, 66.978 mmol, 1.0 equiv) in formic acid (60 mL, 325.910 mmol) was stirred for 12 h at 75° C. The mixture was basified to pH 8 with a saturated solution of Na₂CO₃ and extracted with DCM (3×200 mL). The combined organic layers were washed with brine (2×200 mL), dried over anhydrous Na₂SO₄, filtered and concentrated under reduced pressure. This resulted in (1R,3S)-3-(5-{[(benzyloxy)carbonyl]amino}-1H-pyrazol-3-yl)cyclopentyl 4-nitrophenyl carbonate (30.0 g, 96.0%) as an orange oil. MS (ESI) calcd. for C₂₃H₂₂N₄O₇, 466.15 m/z, found 467.15 [M+H]⁺.

### Example 1A.4: Synthesis of Intermediate D: (1R,3S)-3-[5-(2-bromoacetamido)-1-tert-butylpyrazol-3-yl]cyclopentyl N-isopropylcarbamate

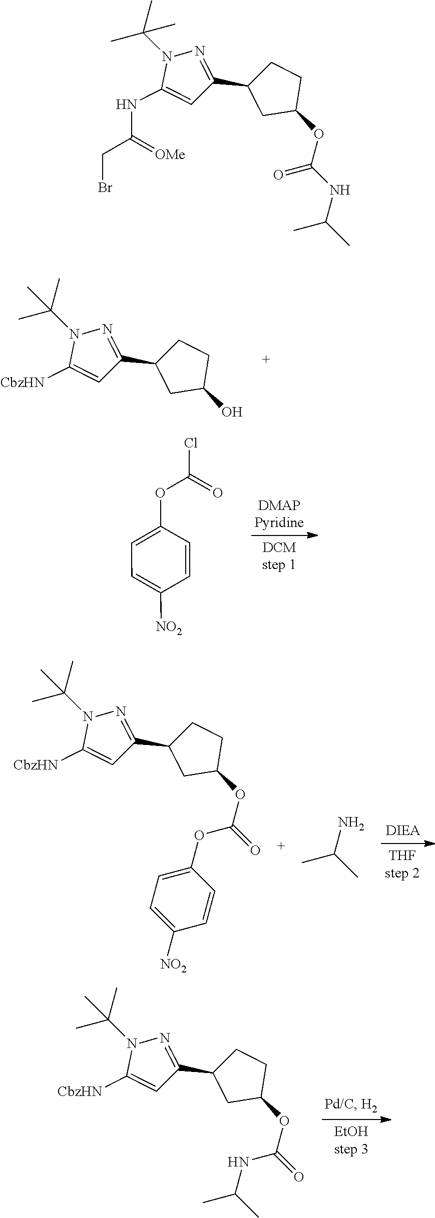

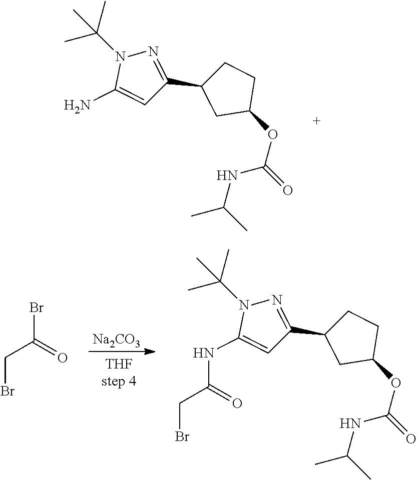

### Step 1: Synthesis of benzyl (1-(tert-butyl)-3-((1S,3R)-3-(((4-nitrophenoxy)carbonyl)oxy)cyclopentyl)-1H-pyrazol-5-yl)carbamate

A solution of benzyl (1-(tert-butyl)-3-((1S,3R)-3-hydroxycyclopentyl)-1H-pyrazol-5-yl)carbamate (15 g, 41.963 mmol, 1 equiv) and 4-nitrophenyl carbonochloridate (9.7 g, 48.257 mmol, 1.15 equiv) in 300 mL DCM was stirred at 25° C. for 10 mins followed by the addition of DMAP (0.51 g, 4.196 mmol, 0.1 equiv) and pyridine (9.96 g, 125.889 mmol, 3 equiv). The resulting mixture was stirred at 25° C. for 20 h, the reaction was checked by LCMS and showed complete consumption of starting material. The resulting mixture was diluted with 200 mL DCM and washed with 100 mL water (3×), the organic phase was separated and dried over anhydrous sodium sulfate. After filtration, the filtrate was concentrated under reduced pressure. The residue was purified by silica gel column chromatography, 0 to 40% EA/PE to obtain benzyl (1-(tert-butyl)-3-((1S,3R)-3-(((4-nitrophenoxy)carbonyl)oxy)cyclopentyl)-1H-pyrazol-5-yl)carbamate (22 g, 80.3% yield). MS (ESI) calcd. for C₂₇H₃N₄O₇, 522.21 m/z, found [M+H]⁺ 523.20 m/z.

### Step 2: Synthesis of (1R,3S)-3-(5-(((benzyloxy)carbonyl)amino)-1-(tert-butyl)-1H-pyrazol-3-yl)cyclopentyl isopropylcarbamate

A solution of benzyl (1-(tert-butyl)-3-((1S,3R)-3-(((4-nitrophenoxy)carbonyl)oxy)cyclopentyl)-1H-pyrazol-5-yl)carbamate (22 g, 42 mmol, 1 equiv), propan-2-amine (3.7 g, 63.151 mmol, 1.5 equiv) and DIEA (16.3 g, 126.303 mmol, 3.0 equiv) in 200 mL THF was stirred at 25° C. for 16 h, the reaction was checked by LCMS and showed complete consumption of starting material. The mixture was concentrated and the residue was purified by silica gel column chromatography, 0 to 30% DCM/EA to afford (1R,3S)-3-(5-(((benzyloxy)carbonyl)amino)-1-(tert-butyl)-1H-pyrazol-3-yl)cyclopentyl isopropylcarbamate (10 g, 48.30% yield). MS (ESI) calcd. for C₂₄H₃₄N₄O₄, 442.26 m/z, found [M+H]⁺ 443.25 m/z.

### Step 3: Synthesis of (1R,3S)-3-(5-amino-1-(tert-butyl)-1H-pyrazol-3-yl)cyclopentyl isopropylcarbamate

(1R,3S)-3-(5-(((benzyloxy)carbonyl)amino)-1-(tert-butyl)-1H-pyrazol-3-yl)cyclopentyl isopropylcarbamate (10.8 g, 24.403 mmol, 1.0 equiv), Pd/C (3 g, 10% on active carbon) and 200 mL EtOH was added to a 500 mL round-bottom flask with a stir bar and stirred at 25° C. for 20 h under H₂, the reaction was checked by LCMS and showed complete consumption of starting material. The mixture was concentrated under reduced pressure to afford ethyl 6-hydroxy-4-methoxy-2,3-dihydro-1H-indene-2-carboxylate (1R,3S)-3-(5-amino-1-tert-butylpyrazol-3-yl)cyclopentyl N-isopropylcarbamate (6.5 g, 79.45% yield) as a white solid which was used in the next step directly without further purification. MS (ESI) calcd. for C₁₆H₂₈N₄O₂, 308.22 m/z, found [M+H]⁺ 309.15 m/z.

### Step 4: Synthesis of Intermediate D, (1R,3S)-3-(5-(2-bromoacetamido)-1-(tert-butyl)-1H-pyrazol-3-yl)cyclopentyl isopropylcarbamate

To a solution of ethyl 6-hydroxy-4-methoxy-2,3-dihydro-1H-indene-2-carboxylate (1R,3S)-3-(5-amino-1-tert-butylpyrazol-3-yl)cyclopentyl N-isopropylcarbamate (2.5 g, 8.106 mmol, 1.0 equiv) and Na₂CO₃ (1.5 g, 14.591 mmol, 1.8 equiv) in 10 mL THF was added 2-bromoacetyl bromide (3.8 g, 16.212 mmol, 2.0 equiv) dropwise and stirred at 25° C. for 2 h under N₂ atmosphere, the reaction was checked by LCMS and showed complete consumption of starting material. The resulting mixture was diluted with 100 mL EA and washed 3 times with 20 mL water, the organic phase was separated and dried over anhydrous sodium sulfate. After filtration, the filtrate was concentrated under reduced pressure. The residue was purified silica gel column chromatography, 0 to 43% EA/PE to afford (1R,3S)-3-(5-(2-bromoacetamido)-1-(tert-butyl)-1H-pyrazol-3-yl)cyclopentyl isopropylcarbamate (2.7 g, 69.82% yield). MS (ESI) calcd. for C₁₈H₂₉BrN₄O₃, 428.14 m/z, found [M+H]⁺ 429.10 m/z.

### Example 1A.5: Synthesis of Intermediate E: (1R,3S)-3-(3-amino-1H-pyrazol-5-yl)cyclopentyl isopropylcarbamate

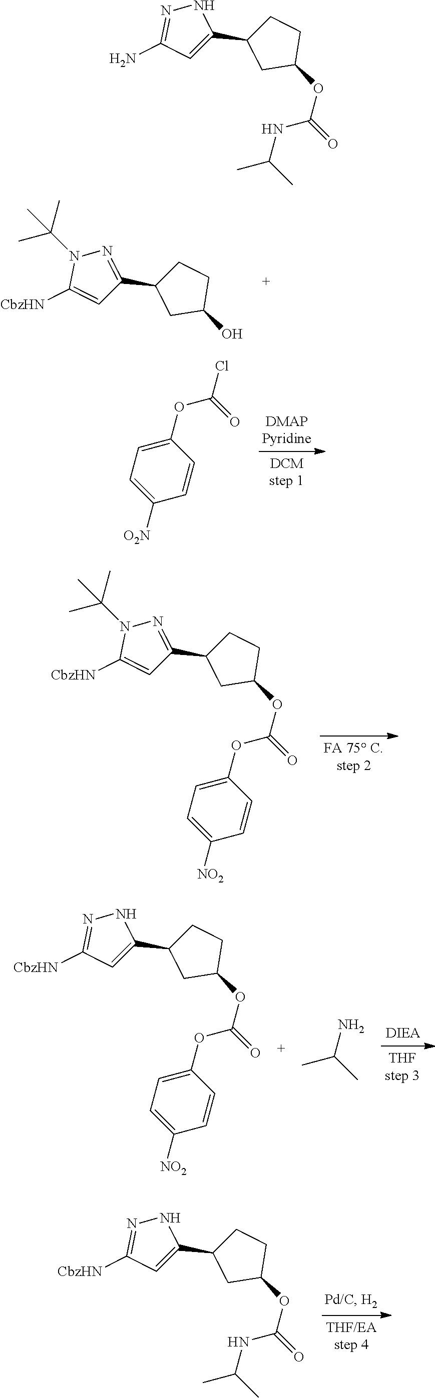

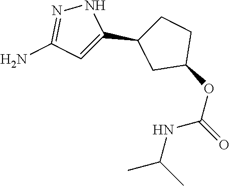 [SMILES: CC(C)NC(=O)O[C@@H]1CC[C@H](c2cc(N)n[nH]2)C1]

### Step 1: Synthesis of (1R,3S)-3-(5-{[(benzyloxy)carbonyl]amino}-1-tert-butylpyrazol-3-yl)cyclopentyl 4-nitrophenyl carbonate

To a stirred solution of benzyl N-{2-tert-butyl-5-[(1S,3R)-3-hydroxycyclopentyl]pyrazol-3-yl}carbamate (25.0 g, 69.939 mmol, 1.0 equiv) and 4-nitrophenyl chloroformate (16.35 g, 81.129 mmol, 1.16 equiv) in DCM (300 mL) was added pyridine (16.97 mL, 209.817 mmol, 3 equiv) and DMAP (0.85 g, 6.994 mmol, 0.10 equiv) at room temperature. The resulting mixture was stirred for 4 h, then diluted with water (100 mL) and extracted with DCM (3×500 mL). The combined organic layers were washed with brine (3×500 mL) and dried over anhydrous Na₂SO₄. After filtration, the filtrate was concentrated under reduced pressure. This resulted in (1R,3S)-3-(5-{[(benzyloxy)carbonyl]amino}-1-tert-butylpyrazol-3-yl)cyclopentyl 4-nitrophenyl carbonate (35.0 g, 95.8%) as a yellow oil. MS (ESI) calcd. for C₂₇H₃₀N₄O₇, 522.21 m/z, found 523.15 [M+H]⁺.

### Step 2: Synthesis of (1R,3S)-3-(5-{[(benzyloxy)carbonyl]amino}-1H-pyrazol-3-yl)cyclopentyl 4-nitrophenyl carbonate

A solution of (1R,3S)-3-(5-{[(benzyloxy)carbonyl]amino}-1-tert-butylpyrazol-3-yl)cyclopentyl 4-nitrophenyl carbonate (35.0 g, 66.978 mmol, 1.0 equiv) in formic acid (60 mL, 325.910 mmol) was stirred for 12 h at 75° C. The mixture was basified to pH 8 with sat. Na₂CO₃ solution. The resulting mixture was extracted with DCM (3×200 mL). The combined organic layers were washed with brine (2×200 mL) and dried over anhydrous Na₂SO₄. After filtration, the filtrate was concentrated under reduced pressure. This resulted in (1R,3S)-3-(5-{[(benzyloxy)carbonyl]amino}-1H-pyrazol-3-yl)cyclopentyl 4-nitrophenyl carbonate (30.0 g, 96.0%) as an orange oil. MS (ESI) calcd. for C₂₃H₂₂N₄O₇, 466.15 m/z, found 467.15 [M+H]⁺.

### Step 3: Synthesis of (1R,3S)-3-(3-(((benzyloxy)carbonyl)amino)-1H-pyrazol-5-yl)cyclopentyl isopropylcarbamate

To a stirred solution of (1R,3S)-3-(5-{[(benzyloxy)carbonyl]amino}-2H-pyrazol-3-yl)cyclopentyl 4-nitrophenyl carbonate (14 g, 30.014 mmol, 1.0 equiv) in THF (140 mL) was added isopropylamine (0.57 mL, 9.648 mmol, 2.0 equiv) and DIEA (2.52 mL, 14.472 mmol, 3.0 equiv) at room temperature. The resulting mixture was stirred for 2 h. The reaction was basified with addition of NaOH 0.5 M solution (200 mL) at room temperature. The resulting mixture was extracted with EA (3×200 mL). The combined organic layers were washed with brine (2×200 mL), dried over anhydrous Na₂SO₄. After filtration, the filtrate was concentrated under reduced pressure. The crude product was purified by silica gel column chromatography, eluted with EA:PE (1:5) to afford benzyl N-{5-[(1S,3R)-3-[(isopropylcarbamoyl)oxy]cyclopentyl]-1H-pyrazol-3-yl}carbamate (8 g, 69.0%) as a yellow solid. MS (ESI) calcd. for C₂₀H₂₆N₄O₄, 386.20 m/z, found 387.20 [M+H]⁺.

### Step 4: Synthesis of Intermediate E, (1R,3S)-3-(3-amino-1H-pyrazol-5-yl)cyclopentyl isopropylcarbamate

To a stirred solution of benzyl N-{5-[(1S,3R)-3-[(isopropylcarbamoyl)oxy]cyclopentyl]-1H-pyrazol-3-yl}carbamate (8 g, 20.701 mmol, 1 equiv) in THF (30 mL) and EA (60 mL) was added Pd/C (2.5 g, 10%) at room temperature under H₂ atmosphere. The resulting mixture was stirred for 2 h. The resulting mixture was filtered, the filter cake was washed with EA (4×100 mL). The filtrate was concentrated under reduced pressure. This resulted in (1R,3S)-3-(3-amino-1H-pyrazol-5-yl)cyclopentyl isopropylcarbamate (4 g, 76.9%) as a yellow solid. MS (ESI) calcd. for C₁₂H₂₀N₄O₂, 252.16 m/z, found 253.20 [M+H]⁺.

### Example 1A.6: Synthesis of (R)-2-(3-(benzyloxy)-2-formyl-5-methoxyphenoxy)propanoic acid

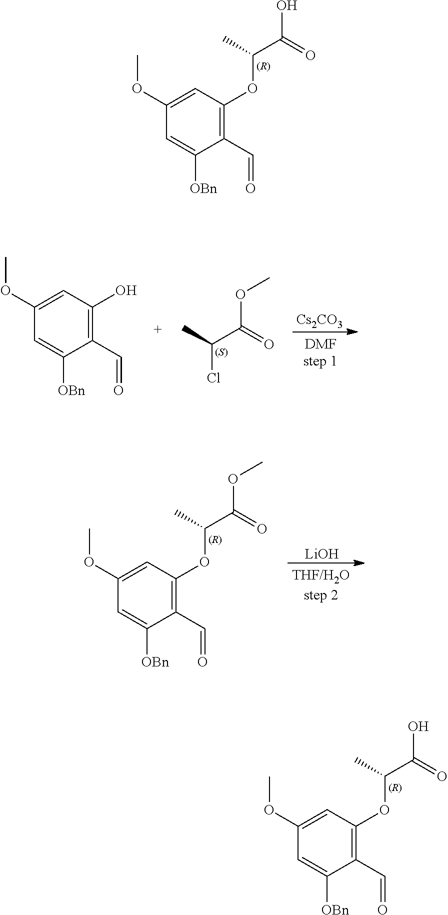

### Step 1: Synthesis of methyl (R)-2-(3-(benzyloxy)-2-formyl-5-methoxyphenoxy)propanoate

To a stirred solution of 2-(bsenzyloxy)-6-hydroxy-4-methoxybenzaldehyde (3.0 g, 11.61 mmol, 1.0 equiv) in 30 mL DMF was added Cs₂CO₃ (9.4 g, 29.04 mmol, 2.5 equiv) at 25° C. and stirred for 0.5 h. Then to the above mixture was added methyl (2S)-2-chloropropanoate (2.1 g, 17.42 mmol, 1.5 equiv) at 25° C. The resulting mixture was stirred for additional 9 h at 25° C., the reaction was checked by LCMS and showed complete consumption of starting material. The mixture was diluted with 30 mL ethyl acetate, washed 3 times with 20 mL water and 30 mL brine, dried over anhydrous sodium sulfate, and concentrated in vacuo. The residue was purified by silica gel column chromatography, 0 to 100% EA/PE to afford methyl 2-(3-(benzyloxy)-2-formyl-5-methoxyphenoxy)propanoate (2.1 g, 50.81% yield) as a yellow oil. Then purified by ANAL_SFC, 0 to 50% MeOH (0.1% DEA) to afford methyl (R)-2-(3-(benzyloxy)-2-formyl-5-methoxyphenoxy)propanoate (1.0 g, 24.20% yield) as a yellow solid. MS (ESI) calcd. for C₁₉H₂₀O₆, 344.13 m/z, found [M+H]⁺ 345.05 m/z.

### Step 2: Synthesis of (R)-2-(3-(benzyloxy)-2-formyl-5-methoxyphenoxy)propanoic acid

To a stirred solution of methyl (R)-2-(3-(benzyloxy)-2-formyl-5-methoxyphenoxy)propanoate (1.0 g, 2.90 mmol, 1.0 equiv) in 10 mL THF and 2 mL H₂O was added LiOH (83.5 mg, 3.48 mmol, 1.2 equiv), then stirred at 25° C. for 2 h, the reaction was checked by LCMS and showed complete consumption of starting material. The mixture was acidified to pH 7 with 1 N HCl at 0° C. The mixture was diluted with 30 mL ethyl acetate, washed 3 times with 20 mL water and 10 mL brine, dried over anhydrous sodium sulfate, and concentrated in vacuo to afford (R)-2-(3-(benzyloxy)-2-formyl-5-methoxyphenoxy)propanoic acid (750.0 mg, 78.18% yield) as a light yellow solid. MS (ESI) calcd. for C₁₈H₁₈O₆, 330.11 m/z, found [M+H]⁺ 331.15 m/z. The crude material was used without any further purification.

### Example 1A.7: Synthesis of (1s,3s)-3-(3-amino-1H-pyrazol-5-yl)cyclobutyl 3-oxa-6-azabicyclo[3.1.1]heptane-6-carboxylate

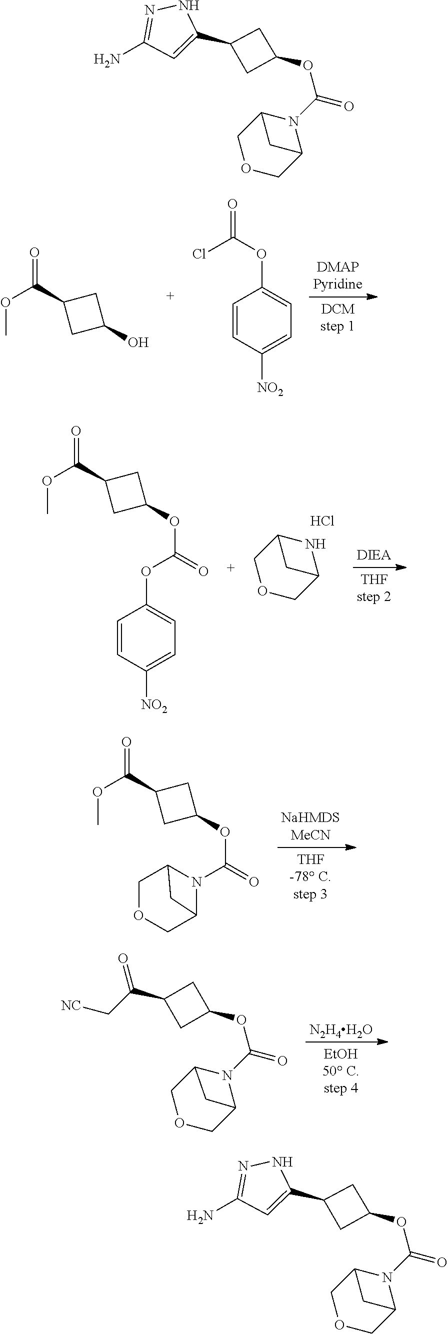

### Step 1: Synthesis of methyl (1s,3s)-3-(((4-nitrophenoxy)carbonyl)oxy)cyclobutane-1-carboxylate

Methyl (1s,3s)-3-hydroxycyclobutane-1-carboxylate (15 g, 115.258 mmol, 1 equiv), DMAP (1.41 g, 11.526 mmol, 0.1 equiv), pyridine (27.82 mL, 345.774 mmol, 3.00 equiv), astir bar and DCM (150 mL) were added to a 500 mL flask and stirred until homogenous, then treated with 4-nitrophenyl carbonochloridate (27.88 g, 138.310 mmol, 1.2 equiv) in portions. The resulting mixture was stirred at r.t. overnight. The reaction was quenched with H₂O and extracted with DCM. The combined organic extracts were washed with brine, dried over sodium sulphate, filtered, and concentrated to dryness in vacuo. The residue obtained was purified by silica gel chromatography, 0 to 15% EA/DCM) to afford methyl (1s,3s)-3-[(4-nitrophenoxycarbonyl)oxy]cyclobutane-1-carboxylate (34 g, 95.89 mmol, 83.15 yield) as a yellow semi-solid.

### Step 2: (1s,3s)-3-(methoxycarbonyl)cyclobutyl 3-oxa-6-azabicyclo[3.1.1]heptane-6-carboxylate

Methyl (1s,3s)-3-[(4-nitrophenoxycarbonyl)oxy]cyclobutane-1-carboxylate (24 g, 67.632 mmol, 1 equiv, 83.2%), a stir bar, DIEA (43.7 g, 338.112 mmol, 5.00 equiv) and THF (200 mL) were added to a 500 mL flask and stirred until homogenous, then treated with 3-oxa-6-azabicyclo[3.1.1]heptane hydrochloride (10.1 g, 74.489 mmol, 1.10 equiv). The resulting mixture was stirred for 2 h. The reaction was quenched with H₂O, and extracted with EA. The combined organic extracts were washed with 0.5 M NaOH, dried over sodium sulphate, filtered, and concentrated to dryness in vacuo. The residue obtained was purified by alkaline silica gel chromatography, 0 to 5% MeOH/DCM to afford (1s,3s)-3-(methoxycarbonyl)cyclobutyl 3-oxa-6-azabicyclo[3.1.1]heptane-6-carboxylate (11.8 g, 41.63 mmol, 68.35% yield) as yellow oil.

### Step 3: (1s,3s)-3-(2-cyanoacetyl)cyclobutyl 3-oxa-6-azabicyclo[3.1.1]heptane-6-carboxylate

(1s,3s)-3-(Methoxycarbonyl)cyclobutyl 3-oxa-6-azabicyclo[3.1.1]heptane-6-carboxylate (11.8 g, 41.603 mmol, 1 equiv), acetonitrile (5.12 g, 124.809 mmol, 3 equiv), a stir bar and THF (120 mL) were added to a 500 mL 3-necked round bottom flask, then treated with NaHMDS (31.2 mL, 62.404 mmol, 2M in THF, 1.5 equiv) dropwise at −78° C. under N₂. The resulting mixture was stirred at −78° C. for 3 h. The reaction was quenched with NH₄Cl solution and extracted with EA. The organic layers were dried over sodium sulphate, filtered, and concentrated to dryness in vacuo. The residue obtained was purified by alkaline silica gel chromatography, 0-5% MeOH/DCM to afford (1s,3s)-3-(2-cyanoacetyl)cyclobutyl 3-oxa-6-azabicyclo[3.1.1]heptane-6-carboxylate (11.4 g, 38.85 mmol, 93.3% yield) as yellow oil.

### Step 4: (1s,3s)-3-(3-amino-1H-pyrazol-5-yl)cyclobutyl 3-oxa-6-azabicyclo[3.1.1]heptane-6-carboxylate

(1s,3s)-3-(2-cyanoacetyl)cyclobutyl 3-oxa-6-azabicyclo[3.1.1]heptane-6-carboxylate (11.4 g, 43.136 mmol, 1 equiv), Hydrazine monohydrate (5.40 g, 86.295 mmol, 2.00 equiv, 80%), a stir bar and EtOH (110 mL) were added to a 500 mL flask. The resulting mixture was stirred at 50° C. overnight, then concentrated. The residue obtained was purified by alkaline silica gel chromatography, 0 to 5% MeOH/DCM to afford (1s,3s)-3-(5-amino-2H-pyrazol-3-yl)cyclobutyl 3-oxa-6-azabicyclo[3.1.1]heptane-6-carboxylate (10.8 g, 36.08 mmol, 89.96% yield) as a yellow semi-solid.

### Example 2 Representative Compounds Following General Scheme A

### Example 2.1: Synthesis of Compound 2: (1R,3S)-3-[5-(4-formyl-3-hydroxybenzamido)-2H-pyrazol-3-yl]cyclopentyl N-isopropylcarbamate; trifluoroacetic acid

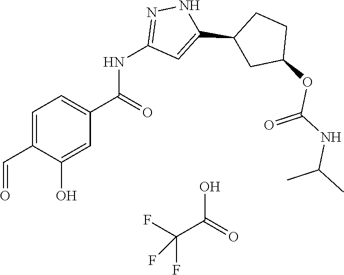 [SMILES: CC(C)NC(=O)O[C@@H]1CC[C@H](c2cc(NC(=O)c3ccc(C=O)c(O)c3)n[nH]2)C1 ; O=C(O)C(F)(F)F]

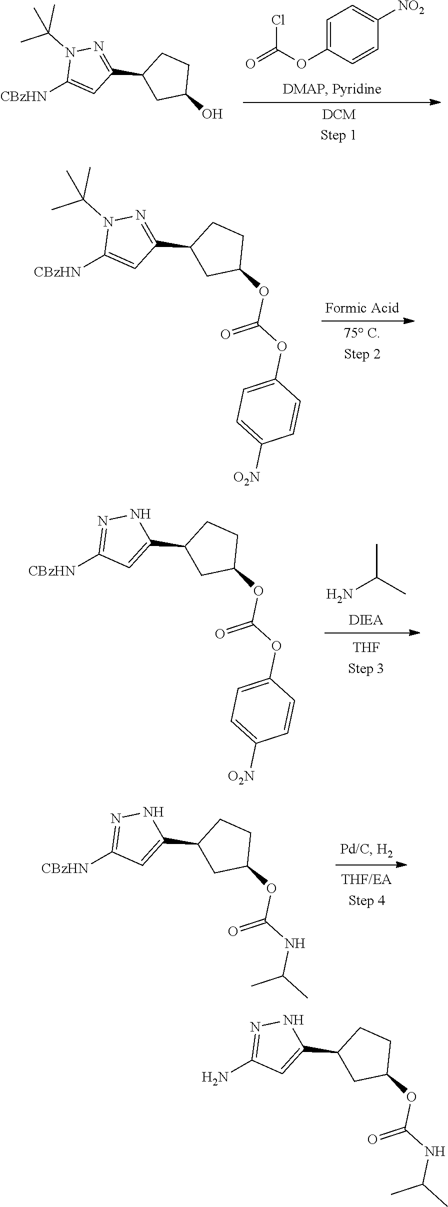

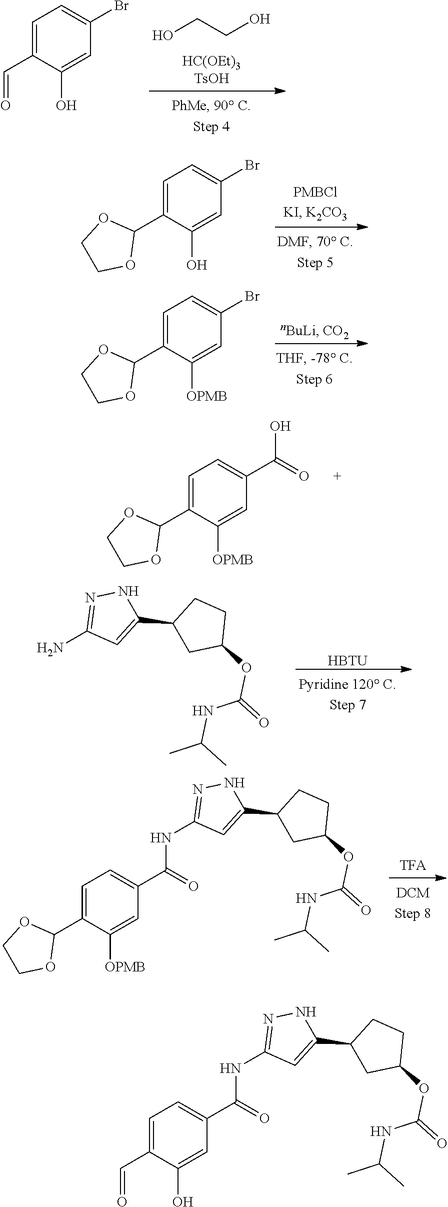

### Step 1: Synthesis of (1R,3S)-3-(5-{[(benzyloxy)carbonyl]amino}-1-tert-butylpyrazol-3-yl)cyclopentyl 4-nitrophenyl carbonate

To a stirred solution of benzyl N-{2-tert-butyl-5-[(1S,3R)-3-hydroxycyclopentyl]pyrazol-3-yl}carbamate (25.0 g, 69.939 mmol, 1.0 equiv) and 4-nitrophenyl chloroformate (16.35 g, 81.129 mmol, 1.16 equiv) in DCM (300 mL) was added pyridine (16.97 mL, 209.817 mmol, 3 equiv) and DMAP (0.85 g, 6.994 mmol, 0.10 equiv) at room temperature. The resulting mixture was stirred for 4 h, then diluted with water (100 mL) and extracted with DCM (3×500 mL). The combined organic layers were washed with brine (3×500 mL), dried over anhydrous Na₂SO₄. After filtration, the filtrate was concentrated under reduced pressure. This resulted in (1R,3S)-3-(5-{[(benzyloxy)carbonyl]amino}-1-tert-butylpyrazol-3-yl)cyclopentyl 4-nitrophenyl carbonate (35.0 g, 95.8%) as a yellow oil. MS (ESI) calcd. for C₂₇H₃₀N₄O₇, 522.21 m/z, found 523.15 [M+H]⁺.

### Step 2: Synthesis of (1R,3S)-3-(5-{[(benzyloxy)carbonyl]amino}-1H-pyrazol-3-yl)cyclopentyl 4-nitrophenyl carbonate

A solution of (1R,3S)-3-(5-{[(benzyloxy)carbonyl]amino}-1-tert-butylpyrazol-3-yl)cyclopentyl 4-nitrophenyl carbonate (35.0 g, 66.978 mmol, 1.0 equiv) in formic acid (60 mL, 325.910 mmol) was stirred for 12 h at 75° C. The mixture was basified to pH 8 with sat. Na₂CO₃ solution. The resulting mixture was extracted with DCM (3×200 mL). The combined organic layers were washed with brine (2×200 mL), dried over anhydrous Na₂SO₄. After filtration, the filtrate was concentrated under reduced pressure. This resulted in (1R,3S)-3-(5-{[(benzyloxy)carbonyl]amino}-1H-pyrazol-3-yl)cyclopentyl 4-nitrophenyl carbonate (30.0 g, 96.0%) as an orange oil. MS (ESI) calcd. for C₂₃H₂₂N₄O₇, 466.15 m/z, found 467.15 [M+H]⁺.

### Step 3: Synthesis of (1R,3S)-3-(3-(((benzyloxy)carbonyl)amino)-1H-pyrazol-5-yl)cyclopentyl isopropylcarbamate

To a stirred solution of (1R,3S)-3-(5-{[(benzyloxy)carbonyl]amino}-2H-pyrazol-3-yl)cyclopentyl 4-nitrophenyl carbonate (14 g, 30.014 mmol, 1.0 equiv) in THF (140 mL) was added isopropylamine (0.57 mL, 9.648 mmol, 2.0 equiv) and DIEA (2.52 mL, 14.472 mmol, 3.0 equiv) at room temperature. The resulting mixture was stirred for 2 h. The reaction was basified with addition of NaOH 0.5 M solution (200 mL) at room temperature. The resulting mixture was extracted with EA (3×200 mL). The combined organic layers were washed with brine (2×200 mL), dried over anhydrous Na₂SO₄. After filtration, the filtrate was concentrated under reduced pressure. The crude product was purified by silica gel column chromatography, eluted with EA:PE (1:5) to afford benzyl N-{5-[(1S,3R)-3-[(isopropylcarbamoyl)oxy]cyclopentyl]-1H-pyrazol-3-yl}carbamate (8 g, 69.0%) as a yellow solid. MS (ESI) calcd. for C₂₀H₂₆N₄O₄, 386.20 m/z, found 387.20 [M+H]⁺.

### Step 4: Synthesis of (1R,3S)-3-(3-amino-1H-pyrazol-5-yl)cyclopentyl isopropylcarbamate

To a stirred solution of benzyl N-{5-[(1S,3R)-3-[(isopropylcarbamoyl)oxy]cyclopentyl]-1H-pyrazol-3-yl}carbamate (8 g, 20.701 mmol, 1 equiv) in THF (30 mL) and EA (60 mL) was added Pd/C (2.5 g, 10%) at room temperature under H₂ atmosphere. The resulting mixture was stirred for 2 h. The resulting mixture was filtered, the filter cake was washed with EA (4×100 mL). The filtrate was concentrated under reduced pressure. This resulted in (1R,3S)-3-(5-amino-2H-pyrazol-3-yl)cyclopentyl N-isopropylcarbamate (4 g, 76.9%) as a yellow solid. MS (ESI) calcd. for C₁₂H₂₀N₄O₂, 252.16 m/z, found 253.20 [M+H]⁺.

### Step 5: Synthesis of 5-bromo-2-(1,3-dioxolan-2-yl)phenol

To a stirred solution of 4-bromo-2-hydroxybenzaldehyde (10 g, 49.747 mmol, 1 equiv) and TsOH (0.86 g, 4.994 mmol, 0.10 equiv) in toluene (200 mL) were added ethylene glycol (13.87 mL, 248.735 mmol, 5.0 equiv) and triethyl orthoformate (24.82 mL, 149.241 mmol, 3.0 equiv) dropwise at room temperature. The resulting mixture was stirred for 18 h at 90° C., then cooled to room temperature, diluted with water and extracted with EA (4×100 mL). The combined organic layers were washed with brine (2×100 mL), dried over anhydrous Na₂SO₄. After filtration, the filtrate was concentrated under reduced pressure. The residue was purified by silica gel column chromatography, eluted with EA/PE (1:5) to afford 5-bromo-2-(1,3-dioxolan-2-yl)phenol (11.5 g, 71.79%) as light yellow oil. MS (ESI) calcd. for C₉H₉BrO₃, 243.97 m/z, found 245.00 [M+H]⁺.

### Step 6: Synthesis of 2-(4-bromo-2-((4-methoxybenzyl)oxy)phenyl)-1,3-dioxolane

To a stirred solution of 5-bromo-2-(1,3-dioxolan-2-yl)phenol (6.7 g, 27.339 mmol, 1 equiv) in DMF (120 mL) was added K₂CO₃ (18.89 g, 136.695 mmol, 5 equiv), KI (0.91 g, 5.468 mmol, 0.2 equiv) and PMBCl (5.14 g, 32.807 mmol, 1.2 equiv) in portions at room temperature. The mixture was stirred for 2 h at 70° C., then cooled to room temperature, diluted with water, and extracted with EA (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with EA/PE (33%) to afford 2-{4-bromo-2-[(4-methoxyphenyl)methoxy]phenyl}-1,3-dioxolane (7.3 g, 66.72%) as a white solid. MS (ESI) calcd. for C₁₇H₁₇BrO₄, 364.03 m/z, found 363.05 [M−H

### Step 7: Synthesis of 4-(1,3-dioxolan-2-yl)-3-[(4-methoxyphenyl)methoxy]benzoic acid

To a stirred solution of 2-{4-bromo-2-[(4-methoxyphenyl)methoxy]phenyl}-1,3-dioxolane (800.0 mg, 2.190 mmol, 1 equiv) in THF (8 mL, 98.742 mmol) was added n-BuLi 2.5 M in hexanes (1.05 mL, 2.628 mmol, 1.2 equiv) dropwise at −78° C. under N₂ atmosphere. The reaction mixture was allowed to stir for 10 minutes at −78° C., CO₂ (g) was then introduced. The resulting mixture was stirred for 15 minutes. The reaction was quenched with sat. NH₄Cl solution at −78° C. The resulting mixture was let warm to room temperature then extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford 4-(1,3-dioxolan-2-yl)-3-[(4-methoxyphenyl)methoxy]benzoic acid (142.0 mg, 15.69%) as a white solid. MS (ESI) calcd. for C₁₈H₁₈O₆, 330.11 m/z, found 329.00 [M−H]⁻.

### Step 8: Synthesis of (1R,3S)-3-{5-[4-(1,3-dioxolan-2-yl)-3-[(4-methoxyphenyl)methoxy]benzamido]-2H-pyrazol-3-yl}cyclopentyl N-isopropylcarbamate

To a stirred solution of 4-(1,3-dioxolan-2-yl)-3-[(4-methoxyphenyl)methoxy]benzoic acid (142.0 mg, 0.433 mmol, 1 equiv) and (1R,3S)-3-(5-amino-2H-pyrazol-3-yl)cyclopentyl N-isopropyl-carbamate (109.2 mg, 0.433 mmol, 1 equiv) in pyridine (6 mL) were added HBTU (197.0 mg, 0.520 mmol, 1.2 equiv) at room temperature. The resulting mixture was stirred for 2 h at 100° C., then cooled to room temperature, diluted with water, and extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford (1R,3S)-3-{5-[4-(1,3-dioxolan-2-yl)-3-[(4-methoxyphenyl)methoxy]benzamido]-2H-pyrazol-3-yl}cyclopentyl N-isopropylcarbamate (181.0 mg, 63.09%) as a brown solid. MS (ESI) calcd. for C₃₀H₃₆N₄O₇, 564.26 m/z, found 565.25 [M+H]⁺.

### Step 9: Synthesis of (1R,3S)-3-[5-(4-formyl-3-hydroxybenzamido)-2H-pyrazol-3-yl]cyclopentyl N-isopropylcarbamate; trifluoroacetic acid

To a stirred solution of (1R,3S)-3-{5-[4-(1,3-dioxolan-2-yl)-3-[(4-methoxyphenyl)methoxy]benzamido]-2H-pyrazol-3-yl}cyclopentyl N-isopropylcarbamate (100.0 mg, 0.177 mmol, 1 equiv) in DCM (3 mL) was added TFA (0.5 mL) dropwise at room temperature. The resulting mixture was stirred for 2 h. The resulting mixture was concentrated under vacuum. The crude product was purified by Prep-HPLC with the following conditions (Column: XBridge Shield RP18 OBD Column, 30×150 mm, 5 μm; Mobile Phase A: Water (0.05% TFA), Mobile Phase B: ACN; Flow rate: 60 mL/min; Gradient: 24% B to 54% B in 7 min, 54% B; Wave Length: 254 nm; RT1 (min): 6.78) to afford (1R,3S)-3-[5-(4-formyl-3-hydroxybenzamido)-2H-pyrazol-3-yl]cyclopentyl N-isopropylcarbamate; trifluoroacetic acid (11.3 mg, 15.88%) as a light yellow solid. MS (ESI) calcd. for C₂₀H₂₄N₄O₅, 400.17 m/z, found 401.20 [M+H]⁺. ¹H NMR (400 MHz, DMSO-d₆) δ 10.95 (br s, 1H), 10.89 (br s, 1H), 10.34 (s, 1H), 7.72 (d, J=8.4 Hz, 1H), 7.50-7.51 (m, 2H), 6.97-6.99 (m, 1H), 6.44 (s, 1H), 5.02-5.03 (m, 1H), 3.56-3.61 (m, 1H), 3.07-3.11 (m, 1H), 2.46-2.48 (m, 1H), 2.01-2.07 (m, 1H), 1.88-1.93 (m, 1H), 1.70-1.78 (m, 2H), 1.60-1.66 (m, 1H), 1.04 (d, J=6.4 Hz, 6H). ¹⁹F NMR (376 MHz, DMSO-d₆) δ −74.71.

### Example 1.2: Synthesis of Compound 3: (1R,3S)-3-(3-(3-formyl-4-hydroxybenzamido)-1H-pyrazol-5-yl)cyclopentyl isopropylcarbamate trifluoroacetic acid

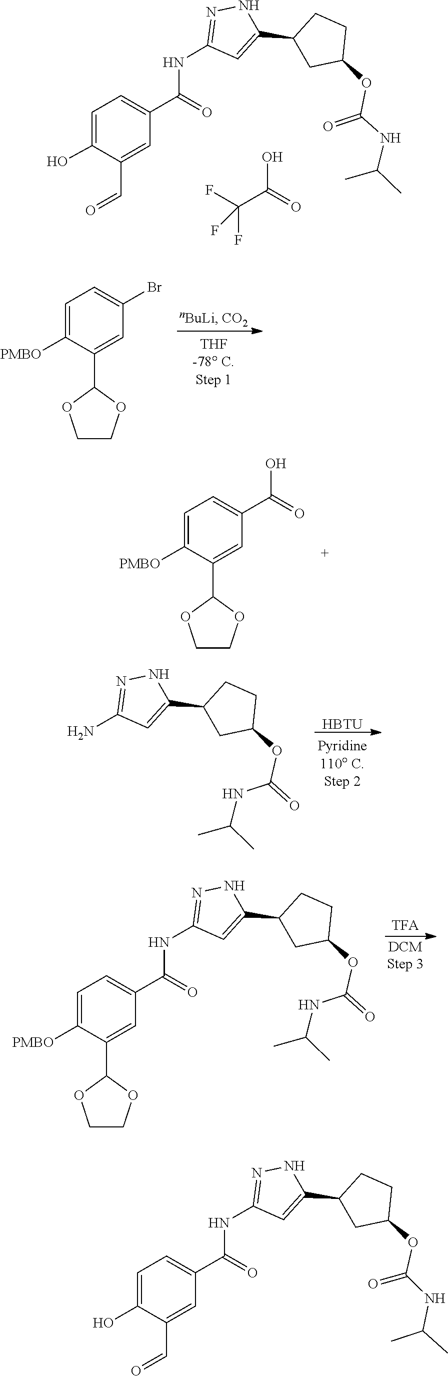

### Step 1: Synthesis of 3-(1,3-dioxolan-2-yl)-4-((4-methoxybenzyl)oxy)benzoic acid

To a stirred solution of 2-{5-bromo-2-[(4-methoxyphenyl)methoxy]phenyl}-1,3-dioxolane (500.0 mg, 1.369 mmol, 1 equiv) in THF (8 mL) was added n-BuLi (0.66 mL, 1.643 mmol, 1.2 equiv) dropwise at −78° C. under N₂ atmosphere. After 10 minutes, CO₂ was added. The resulting mixture was stirred for 15 minutes. The reaction was quenched with sat. NH₄Cl solution at −78° C. The resulting mixture was extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford 3-(1,3-dioxolan-2-yl)-4-[(4-methoxyphenyl)methoxy]benzoic acid (160.0 mg, 35.38%) as a white solid. MS (ESI) calcd. for C₁₈H₁₈O₆, 330.11 m/z, found 329.00 [M−H]⁻.

### Step 2: Synthesis of (1R,3S)-3-(3-(3-(1,3-dioxolan-2-yl)-4-((4-methoxybenzyl)oxy) benzamido)-1H-pyrazol-5-yl)cyclopentyl isopropylcarbamate

To a stirred solution of (1R,3S)-3-(5-amino-2H-pyrazol-3-yl)cyclopentyl N-isopropylcarbamate (110.0 mg, 0.436 mmol, 1 equiv) and 3-(1,3-dioxolan-2-yl)-4-[(4-methoxyphenyl)methoxy]benzoic acid (158.4 mg, 0.480 mmol, 1.1 equiv) in pyridine (4 mL) were added HBTU (198.4 mg, 0.523 mmol, 1.2 equiv) at room temperature. The resulting mixture was stirred for 2 h at 100° C. The resulting mixture was extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford (1R,3S)-3-(3-(3-(1,3-dioxolan-2-yl)-4-((4-methoxybenzyl)oxy)benzamido)-1H-pyrazol-5-yl)cyclopentyl isopropylcarbamate (100.0 mg, 40.62%) as a brown solid. MS (ESI) calcd. for C₃₀H₃₆N₄O₇, 564.26 m/z, found 565.15 [M+H]⁺.

### Step 3: Synthesis of (1R,3S)-3-(3-(3-formyl-4-hydroxybenzamido)-1H-pyrazol-5-yl)cyclopentyl isopropylcarbamate trifluoroacetic acid

To a solution of (1R,3S)-3-{5-[3-(1,3-dioxolan-2-yl)-4-[(4-methoxyphenyl)methoxy]benzamido]-2H-pyrazol-3-yl}cyclopentyl N-isopropylcarbamate (100.0 mg, 0.177 mmol, 1 equiv) in Dichloromethane (2 mL) was added TFA (0.4 mL, 5.385 mmol, 30.41 equiv) dropwise at room temperature. The resulting mixture was stirred for 1 h at room temperature. The reaction progress was monitored by LCMS. The resulting mixture was concentrated under reduced pressure. The crude product was purified by Prep-HPLC with the following conditions: (Column: XBridge Shield RP18 OBD Column, 30×150 mm, 5 μm; Mobile Phase A: Water (0.05% TFA), Mobile Phase B: ACN; Flow rate: 60 mL/min; Gradient: 24% B to 54% B in 7 min; Wave Length: 254 nm; RT1 (min): 6.78; Injection Volume: 0.6 mL; Number Of Runs: 3) to afford (1R,3S)-3-(3-(3-formyl-4-hydroxybenzamido)-1H-pyrazol-5-yl)cyclopentyl isopropylcarbamate; trifluoroacetic acid (14 mg, 14.76%) as a light yellow solid. MS (ESI) calcd. for C₂₀H₂₄N₄O₅, 400.17 m/z, found 401.20 [M+H]⁺. ¹H NMR (400 MHz, DMSO-d₆) δ 11.35 (br s, 1H), 10.81 (s, 1H), 10.32 (s, 1H), 8.35-8.36 (m, 1H), 8.13-8.16 (m, 1H), 7.08 (d, J=8.8 Hz, 1H), 6.97-6.99 (m, 1H), 6.41 (s, 1H), 5.01-5.04 (m, 1H), 3.54-3.63 (m, 1H), 3.04-3.11 (m, 1H), 2.46-2.48 (m, 1H), 2.01-2.07 (m, 1H), 1.86-1.96 (m, 1H), 1.59-1.75 (m, 3H), 1.04 (d, J=6.4 Hz, 6H). ¹⁹F NMR (376 MHz, DMSO-d₆) δ −74.89.

### Example 2.3: Synthesis of Compound 15: (1R,3S)-3-(3-(3-formyl-4-hydroxy-5-methylbenzamido)-1H-pyrazol-5-yl)cyclopentyl isopropylcarbamate trifluoroacetic acid

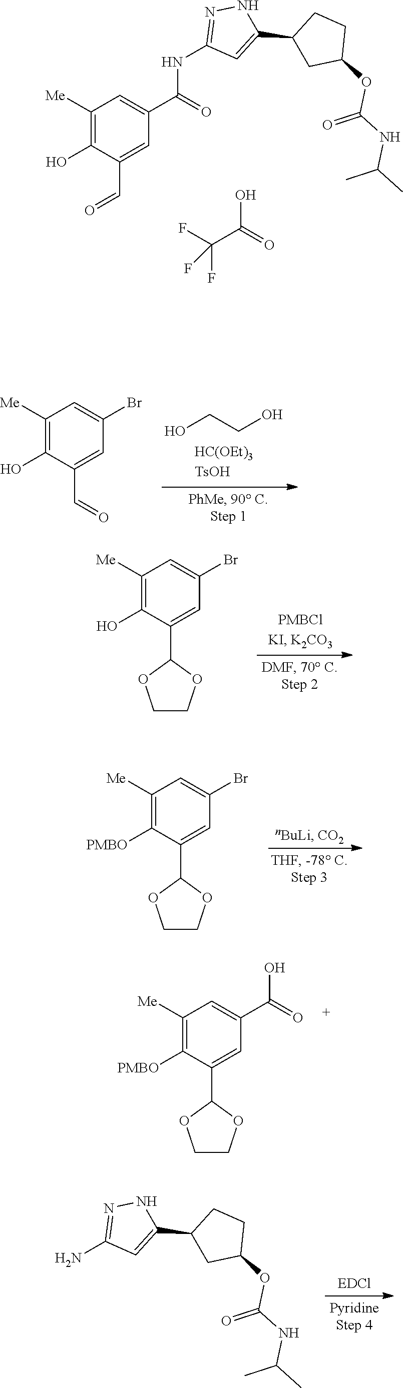

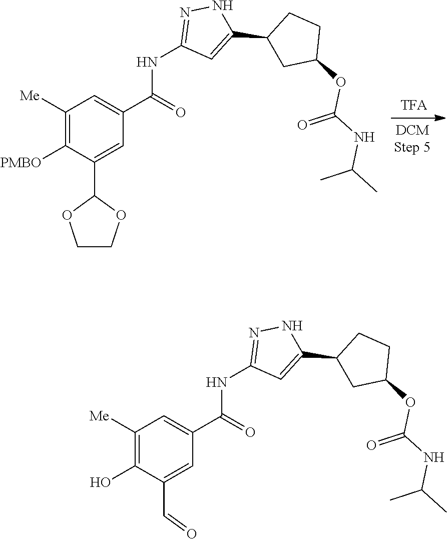

### Step 1: Synthesis of 4-bromo-2-(1,3-dioxolan-2-yl)-6-methylphenol

To a solution of 5-bromo-2-hydroxy-3-methylbenzaldehyde (5 g, 23.251 mmol, 1 equiv) in toluene (100 mL) was added ethylene glycol (7.22 g, 116.255 mmol, 5 equiv), triethyl orthoformate (10.34 g, 69.753 mmol, 3 equiv) and para-toluene sulfonic acid (0.40 g, 2.325 mmol, 0.1 equiv). After stirring for 12 h at 120° C., the resulting mixture was cooled to room temperature, diluted with water and extracted with EA (3×100 mL). The combined organic layers were washed with brine (3×100 mL), dried over anhydrous Na₂SO₄. After filtration, the filtrate was concentrated under reduced pressure. The crude product was purified by silica gel column chromatography, eluted with EA:PE (1:1) to afford 4-bromo-2-(1,3-dioxolan-2-yl)-6-methylphenol (3 g, 49.80%) as a light-yellow oil. MS (ESI) calcd. for C₁₀H₁₁BrO₃, 257.99 m/z, found 259.00 [M+H]⁺.

### Step 2: Synthesis of 2-(5-bromo-2-((4-methoxybenzyl)oxy)-3-methylphenyl)-1,3-dioxolane

To a solution of 4-bromo-2-(1,3-dioxolan-2-yl)-6-methylphenol (2.5 g, 9.649 mmol, 1 equiv) in DMF (50 mL) was added 4-methoxybenzyl chloride (1.81 g, 11.579 mmol, 1.2 equiv), KI (0.32 g, 1.930 mmol, 0.2 equiv) and K₂CO₃ (4.00 g, 28.947 mmol, 3 equiv). After stirring for 3 h at 70° C., the resulting mixture was cooled to room temperature, diluted with water, and extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford 2-{5-bromo-2-[(4-methoxyphenyl)methoxy]-3-methylphenyl}-1,3-dioxolane (3.0 g, 81.98%) as a light yellow oil.

### Step 3: Synthesis of 3-(1,3-dioxolan-2-yl)-4-((4-methoxybenzyl)oxy)-5-methylbenzoic acid

In a 50-mL round bottom flask, to a solution of 2-{5-bromo-2-[(4-methoxyphenyl)methoxy]-3-methylphenyl}-1,3-dioxolane (1.0 g, 2.637 mmol, 1 equiv) in THF (20 mL) was added dropwise n-butyllithium (1.27 mL, 3.164 mmol, 1.2 equiv) solution (2.5 M in THF/hexane), at −78° C. under N₂ atmosphere. The reaction mixture was stirred at −78° C. for 30 mins. Then carbon dioxide was added and the mixture was stirred for another 60 mins. The reaction was quenched with water (10 mL), The mixture was neutralized to pH 6 with 0.1 M HCl. and then the mixture was extracted with EtOAc (3×50 mL). The combined organic extracts were washed with brine (50 mL), dried over anhydrous Na₂SO₄. After filtration, the filtrate was concentrated under reduced pressure and purified by flash chromatography (Petroleum ether/Ethyl acetate=5/95)) to yield 3-(1,3-dioxolan-2-yl)-4-[(4-methoxyphenyl)methoxy]-5-methylbenzoic acid (800.0 mg, 88.10%) as a white solid. MS (ESI) calcd. for C₁₉H₂₀O₆, 344.13 m/z, found 343.05 [M−H]⁻.

### Step 4: Synthesis of (1R,3S)-3-(3-(3-(1,3-dioxolan-2-yl)-4-((4-methoxybenzyl)oxy)-5-methylbenzamido)-1H-pyrazol-5-yl)cyclopentyl isopropylcarbamate

To a stirred solution of 3-(1,3-dioxolan-2-yl)-4-[(4-methoxyphenyl)methoxy]-5-methylbenzoic acid (175.0 mg, 0.508 mmol, 1 equiv) and (1R,3S)-3-(5-amino-2H-pyrazol-3-yl)cyclopentyl N-isopropylcarbamate (128.22 mg, 0.508 mmol, 1 equiv) in pyridine (5 mL) were added HBTU (231.27 mg, 0.610 mmol, 1.2 equiv). The resulting mixture was stirred for 10 h at 110° C., then cooled to room temperature and diluted with water. The resulting mixture was extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford (1R,3S)-3-{5-[3-(1,3-dioxolan-2-yl)-4-[(4-methoxyphenyl)methoxy]-5-methylbenzamido]-2H-pyrazol-3-yl}cyclopentyl N-isopropyl-carbamate (60 mg, 20.40%) as a brown oil. MS (ESI) calcd. for C₃₁H₃₈N₄O₇, 578.27 m/z, found 579.30 [M+H]⁺.

### Step 5: Synthesis of (1R,3S)-3-(3-(3-formyl-4-hydroxy-5-methylbenzamido)-1H-pyrazol-5-yl)cyclopentyl isopropylcarbamate trifluoroacetic acid

To a stirred solution of (1R,3S)-3-{5-[3-(1,3-dioxolan-2-yl)-4-[(4-methoxyphenyl)methoxy]-5-methylbenzamido]-2H-pyrazol-3-yl}cyclopentyl N-isopropylcarbamate (40.0 mg, 0.069 mmol, 1 equiv) in DCM (1 mL, 15.731 mmol, 227.57 equiv) was added TFA (1 mL, 13.463 mmol, 194.77 equiv) dropwise at room temperature. The resulting mixture was stirred for 0.5 h. The resulting mixture was concentrated under reduced pressure. The crude product was purified by Prep-HPLC with the following conditions (Column: Xselect CSH C18 OBD Column, 30×150 mm, 5 μm; Mobile Phase A: Water (0.1% FA), Mobile Phase B: ACN; Flow rate: 60 mL/min; Gradient: 25% B to 50% B in 10 min; Wave Length: 254 nm; RT1 (min): 9) to afford (1R,3S)-3-[5-(3-formyl-4-hydroxy-5-methylbenzamido)-2H-pyrazol-3-yl]cyclopentyl N-isopropylcarbamate (6.9 mg, 23.92%) as a white solid. MS (ESI) calcd. for C₂₁H₂₆N₄O₅, 414.19 m/z, found 415.20 [M+H]⁺. ¹H NMR (300 MHz, DMSO-d₆) δ 11.27 (br s, 1H), 10.72 (s, 1H), 10.09 (s, 1H), 8.31 (d, J=2.4 Hz, 1H), 8.09-8.20 (m, 1H), 6.95 (d, J=7.2 Hz, 1H), 6.41 (s, 1H), 4.86-5.10 (m, 1H), 3.47-3.70 (m, 1H), 2.99-3.26 (m, 1H), 2.41-2.48 (m, 1H), 2.25 (s, 3H), 1.82-2.09 (m, 2H), 1.56-1.81 (m, 3H), 1.02 (d, J=6.3 Hz, 6H). ¹⁹F NMR (282 MHz, DMSO-d₆) δ −74.97.

### Example 2.4: Synthesis of Compound 26: (1R,3S)-3-{5-[(2R)-4-formyl-5-hydroxy-2,3-dihydro-1H-indene-2-amido]-2H-pyrazol-3-yl}cyclopentyl N-isopropylcarbamate

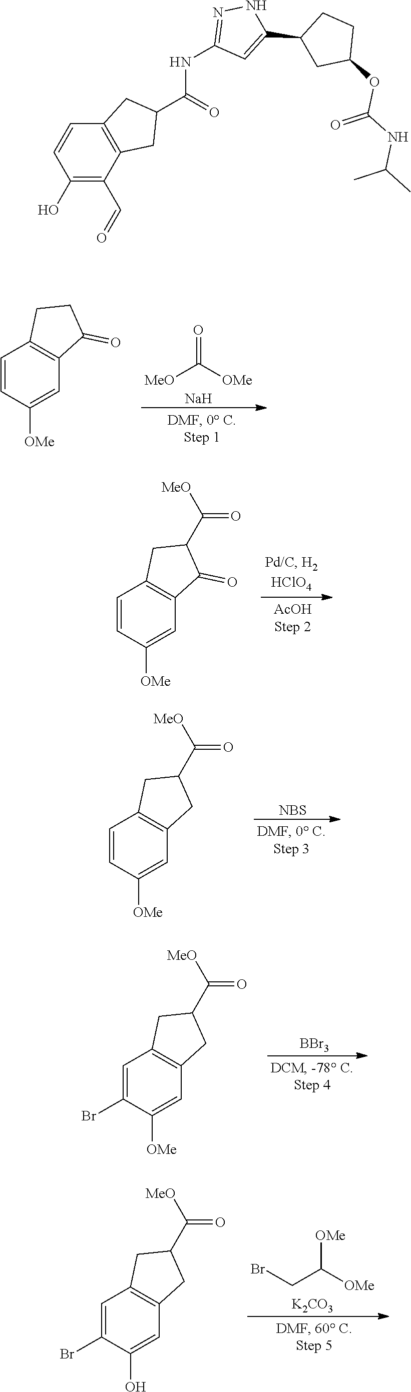

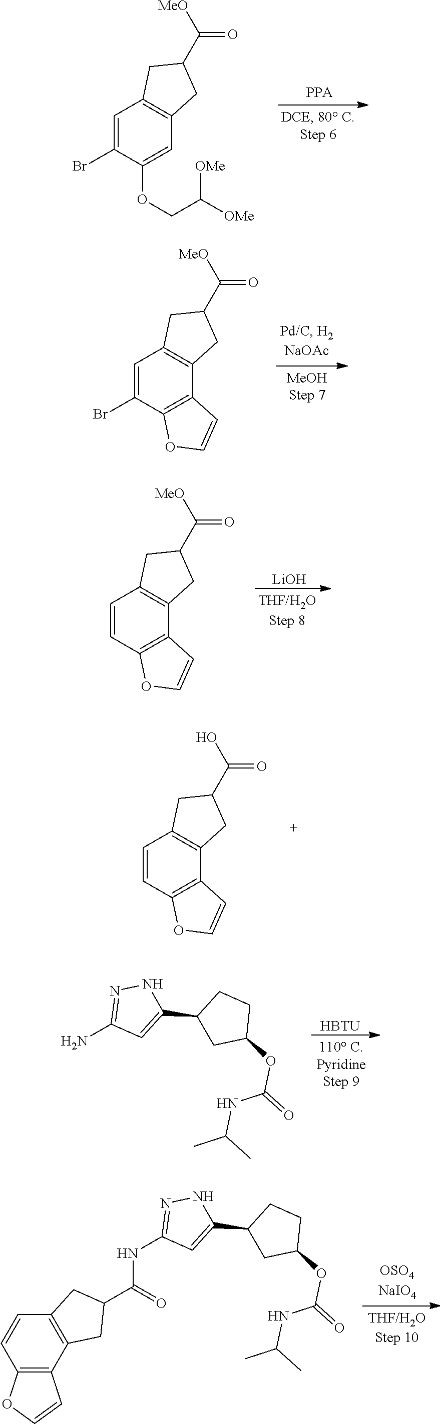

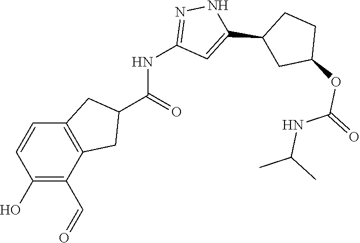 [SMILES: CC(C)NC(=O)O[C@@H]1CC[C@H](c2cc(NC(=O)C3Cc4ccc(O)c(C=O)c4C3)n[nH]2)C1]

### Step 1: methyl 6-methoxy-1-oxo-2,3-dihydroindene-2-carboxylate

A solution of 6-methoxy-2,3-dihydroinden-1-one (15 g, 92.485 mmol, 1 equiv) in DMF (50 mL) was treated with NaH (2.6 g, 110.982 mmol, 1.2 equiv) for 0.5 h at 0° C. under nitrogen atmosphere followed by the addition of dimethyl carbonate (10.00 g, 110.982 mmol, 1.2 equiv) dropwise at 0° C. The resulting mixture was stirred for 12 h at 25° C. under N₂ atmosphere. The reaction was quenched with NH₄Cl (30 mL) at 25° C. The resulting mixture was diluted with EA (100 mL) and washed with H₂O (50 mL×3), dried over anhydrous Na₂SO₄. After filtration, the filtrate was concentrated under reduced pressure. Then the residue was dissolved in ACN (100 mL) and stirred at 25° C. for 8 hours, the mixture was filtered, and the filtrate was concentrated under vacuum. The residue was purified by silica gel column chromatography, eluted with PE/EA (0-60%) to afford methyl 6-methoxy-1-oxo-2,3-dihydroindene-2-carboxylate (14 g, 62.55%) as a yellow solid. MS (ESI) calcd. for C₁₂H₁₂O₄, 220.02 m/z, found 221.20 [M+H]⁺.

### Step 2: methyl 5-methoxy-2,3-dihydro-1H-indene-2-carboxylate

A solution of methyl 6-methoxy-1-oxo-2,3-dihydroindene-2-carboxylate (9.3 g, 42.230 mmol, 1 equiv) and Pd/C (5 g, 46.984 mmol, 1.11 equiv) and HCIO₄ (0.4 mL, 0.423 mmol, 0.01 equiv) in AcOH (40 mL) was stirred at 25° C. for 12 h under H₂ atmosphere. The resulting mixture was filtered, the filter cake was washed with EA (100 mL×3). The filtrate was concentrated under reduced pressure. The residue was purified by silica gel column chromatography, eluted with PE/EA (0-70%) to afford methyl 5-methoxy-2,3-dihydro-1H-indene-2-carboxylate (9 g, 87.84%) as a yellow solid. MS (ESI) calcd. for C₁₂H₁₄O₃, 206.09 m/z, found 207.13 [M+H]⁺.

### Step 3: methyl 5-bromo-6-methoxy-2,3-dihydro-1H-indene-2-carboxylate

A solution of methyl 5-methoxy-2,3-dihydro-1H-indene-2-carboxylate (5.27 g, 25.553 mmol, 1 equiv) in DMF (50 mL) was cooled 0° C. and let stir for 5 min under nitrogen atmosphere followed by the portion-wise addition of 1-bromopyrrolidine-2,5-dione (5.46 g, 30.664 mmol, 1.2 equiv) at 0° C. The resulting mixture was stirred for 1 h at 25° C. under N₂ atmosphere. The resulting mixture was diluted with EA (50 mL) and washed with H₂O (30 mL×3), dried over anhydrous Na₂SO₄. After filtration, the filtrate was concentrated under reduced pressure. The residue was purified by silica gel column chromatography, eluted with PE/EA (0-50%) to afford methyl 5-bromo-6-methoxy-2,3-dihydro-1H-indene-2-carboxylate (6 g, 74.94%) as a yellow solid. MS(ESI) calcd. for C₁₂H₁₃BrO₃, 284.00 m/z, found 285.47 [M+H]⁺.

### Step 4: methyl 5-bromo-6-hydroxy-2,3-dihydro-1H-indene-2-carboxylate

A solution of methyl 5-bromo-6-methoxy-2,3-dihydro-1H-indene-2-carboxylate (3 g, 10.521 mmol, 1 equiv) in DCM (30 mL) was cooled to −78° C. and let stir for 5 mins under nitrogen atmosphere followed by the addition of boron tribromide (52.6 mL, 52.605 mmol, 5 equiv) dropwise at −78° C. The resulting mixture was stirred for 5 h at 25° C. under N₂ atmosphere. The reaction was quenched with NaHCO₃ (30 mL) at 0° C. The resulting mixture was diluted with EA (50 mL), washed with H₂O (30 mL×3), and dried over anhydrous Na₂SO₄. After filtration, the filtrate was concentrated under reduced pressure. The residue was purified by silica gel column chromatography, eluted with PE/EA (0-70%) to afford methyl 5-bromo-6-hydroxy-2,3-dihydro-1H-indene-2-carboxylate (1.8 g, 57.07%) as a yellow solid. MS (ESI) calcd. for C₁₁H₁₁BrO₃, 269.99 m/z, found 273.08 [M+2+H]⁺.

### Step 5: methyl 5-bromo-6-(2,2-dimethoxyethoxy)-2,3-dihydro-1H-indene-2-carboxylate

A solution of methyl 5-bromo-6-hydroxy-2,3-dihydro-1H-indene-2-carboxylate (1.6 g, 6.086 mmol, 1 equiv) in DMF (30 mL) was treated with K₂CO₃ (3.9 g, 12.172 mmol, 2 equiv) for 0.5 h at 25° C. followed by the addition of ethane, 2-bromo-1,1-dimethoxy- (1.5 g, 9.129 mmol, 1.5 equiv) in portions at 25° C. The resulting mixture was stirred for 12 h at 60° C. Desired product could be detected by LCMS. The resulting mixture was diluted with EA (50 mL) and washed with H₂O (30 mL×3), dried over anhydrous Na₂SO₄. After filtration, the filtrate was concentrated under reduced pressure. The residue was purified by silica gel column chromatography, eluted with PE/EA (0-70%) to afford methyl 5-bromo-6-(2,2-dimethoxyethoxy)-2,3-dihydro-1H-indene-2-carboxylate (1.2 g, 49.40%) as a yellow solid. MS (ESI) calcd. for C₁₅H₁₉BrO₅, 358.04 m/z, found 361.56 [M+2+H]⁺.

### Step 6: methyl 4-bromo-6H,7H,8H-indeno[5,4-b]furan-7-carboxylate

A solution of methyl 5-bromo-6-(2,2-diethoxyethoxy)-2,3-dihydro-1H-indene-2-carboxylate (1.2 g, 3.873 mmol, 1 equiv) and PPA (1.3 g, 11.619 mmol, 3 equiv) in DCE (10 mL) was stirred at 80° C. for 1 h under N₂ atmosphere. The resulting mixture was diluted with DCM (50 mL) and washed with H₂O (30 mL×3), dried over anhydrous Na₂SO₄. After filtration, the filtrate was concentrated under reduced pressure. The residue was purified by silica gel column chromatography, eluted with PE/EA (0-70%) to afford methyl 4-bromo-6H,7H,8H-indeno[5,4-b]furan-7-carboxylate (1.1 g, 86.60%) as a yellow solid. MS (ESI) calcd. for C₁₃H₁BrO₃, 293.99 m/z, found 297.20 [M+2+H]⁺.

### Step 7: methyl 6H,7H,8H-indeno[5,4-b]furan-7-carboxylate

A solution of methyl 4-bromo-6H,7H,8H-indeno[5,4-b]furan-7-carboxylate (400 mg, 1.355 mmol, 1 equiv), NaOAc (111.1 mg, 1.355 mmol, 1 equiv) and 10% Pd(OH)₂/C (399.7 mg) in EA (10 mL) was stirred at 25° C. for 1 h under H₂ atmosphere. The resulting mixture was filtered, the filter cake was washed with EA (30 mL×3). The filtrate was concentrated under reduced pressure. The residue was purified by silica gel column chromatography, eluted with PE/EA (0-50%) to afford methyl 6H,7H,8H-indeno[5,4-b]furan-7-carboxylate (220 mg, 67.56%) as a white oil. MS (ESI) calcd. for C₁₃H₁₂O₃, 216.08 m/z, found 217.04 [M+H]⁺.

### Step 8: 6H,7H,8H-indeno[5,4-b]furan-7-carboxylic acid

A solution of methyl 6H,7H,8H-indeno[5,4-b]furan-7-carboxylate (200 mg, 0.925 mmol, 1 equiv) and LiOH H₂O (97.0 mg, 2.313 mmol, 2.5 equiv) in THF (1 mL) and H₂O (0.2 mL) was stirred at 25° C. for 1 h. The mixture was acidified to pH 7 with HCl (0.5M). The resulting mixture was diluted with EA (50 mL) and washed with H₂O (30 mL×2), dried over anhydrous Na₂SO₄. After filtration, the filtrate was concentrated under reduced pressure to afford 6H,7H,8H-indeno[5,4-b]furan-7-carboxylic acid (180 mg, 86.62%) as a yellow solid. MS (ESI) calcd. for C₁₂H₁₀O₃, 202.06 m/z, found 203.37 [M+H]⁺.

### Step 9: (1R,3S)-3-(5-{6H,7H,8H-indeno[5,4-b]furan-7-amido}-2H-pyrazol-3-yl)cyclopentyl N-isopropylcarbamate

A solution of 6H,7H,8H-indeno[5,4-b]furan-7-carboxylic acid (160 mg, 0.791 mmol, 1.3 equiv) and (1R,3S)-3-(5-amino-2H-pyrazol-3-yl)cyclopentyl N-isopropylcarbamate (153.6 mg, 0.608 mmol, 1 equiv) and HBTU (346.3 mg, 0.913 mmol, 1.5 equiv) in pyridine (5 mL) was stirred at 110° C. for 12 h. The resulting mixture was diluted with EA (30 mL) and washed with H₂O (30 mL×2), dried over anhydrous Na₂SO₄. After filtration, the filtrate was concentrated under reduced pressure. The residue was purified by Prep-TLC (MeOH/DCM 10%) to afford (1R,3S)-3-(5-{6H,7H,8H-indeno[5,4-b]furan-7-amido}-2H-pyrazol-3-yl)cyclopentyl N-isopropylcarbamate (135 mg, 46.24%) as a yellow solid. MS (ESI) calcd. for C₂₄H₂₈N₄O₄, 436.21 m/z, found 437.15 [M+H]⁺.

### Step 10: (1R,3S)-3-{5-[(2R)-4-formyl-5-hydroxy-2,3-dihydro-1H-indene-2-amido]-2H-pyrazol-3-yl}cyclopentyl N-isopropylcarbamate

A solution of (1R,3S)-3-(5-{6H,7H,8H-indeno[5,4-b]furan-7-amido}-2H-pyrazol-3-yl)cyclo-pentyl N-isopropylcarbamate (100 mg, 0.229 mmol, 1 equiv) in THF (1 mL) and H₂O (1 mL) was treated with OsO₄ (1.5 mL, 0.115 mmol, 0.5 equiv) for 1 h at 0° C. under nitrogen atmosphere followed by the addition of NaIO₄ (294.0 mg, 1.374 mmol, 6 equiv) in portions at 0° C. The resulting mixture was stirred for 2 h at 25 under N₂ atmosphere. The resulting mixture was diluted with EA (50 mL) and washed with H₂O (30 mL×2), dried over anhydrous Na₂SO₄. After filtration, the filtrate was concentrated under reduced pressure. The crude product was purified by Prep-HPLC with the following condition: Column; XSelect CSH Prep C18 OBD Column 30×150 mm, 5 μm; Mobile Phase Ax Water (0.1% FA), Mobile Phase B: ACN; Flow rate: 60 mL/min; Gradient: 28% B to 47% B in 10 min; Wave Length: 254/220 nm; RT1: 8.65 min, affording (1R,3S)-3-{5-[(2R)-4-formyl-5-hydroxy-2,3-dihydro-1H-indene-2-amido]-2H-pyrazol-3-yl}cyclopentyl N-isopropylcarbamate (5.6 mg, 5.51%) as a yellow green solid. MS (ESI) calcd. for C₂₃H₂₈N₄O₅, 440.21 m/z, found 441.20 [M+H]⁺. ¹H NMR (300 MHz, DMSO-d₆) δ (ppm): 12.06 (s, 1H), 10.61 (s, 1H), 10.53-10.38 (m, 2H), 7.35 (d, J=8.3 Hz, 1H), 6.94 (d, J=7.8 Hz, 1H), 6.80 (d, J=8.3 Hz, 1H), 6.32 (s, 1H), 4.99 (s, 1H), 3.63-3.36 (m, 3H), 3.32-3.21 (m, 3H), 3.10-2.90 (m, 3H), 2.49-2.38 (m, 1H), 2.11-1.96 (m, 1H), 1.99-1.79 (m, 1H), 1.76-1.67 (m, 2H), 1.59 (s, 2H), 1.03 (d, J=6.6 Hz, 6H).

### Example 2.5: Synthesis of Compound 37: (1R,3S)-3-(3-(3-acetamido-4-formylbenzamido)-1H-pyrazol-5-yl)cyclopentyl isopropylcarbamate

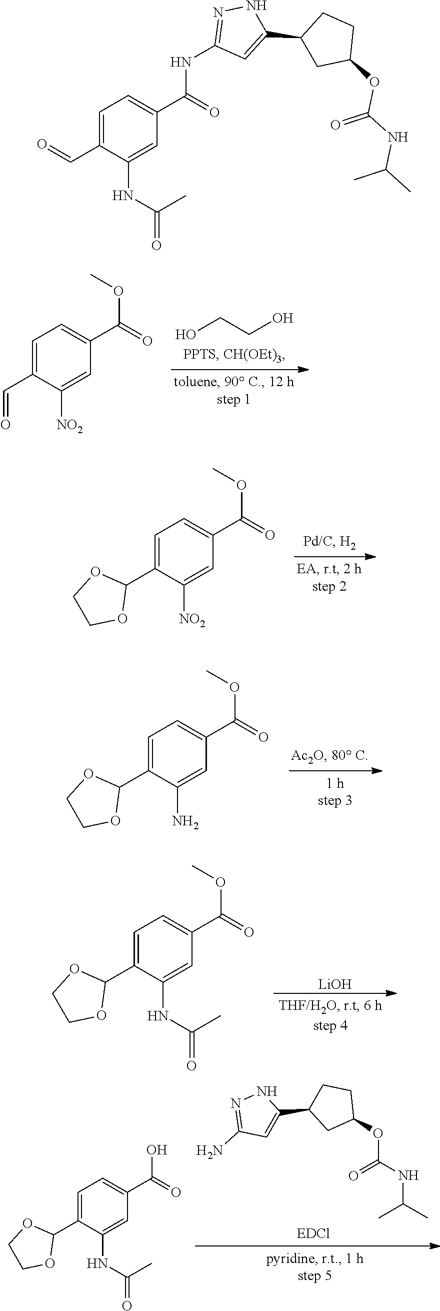

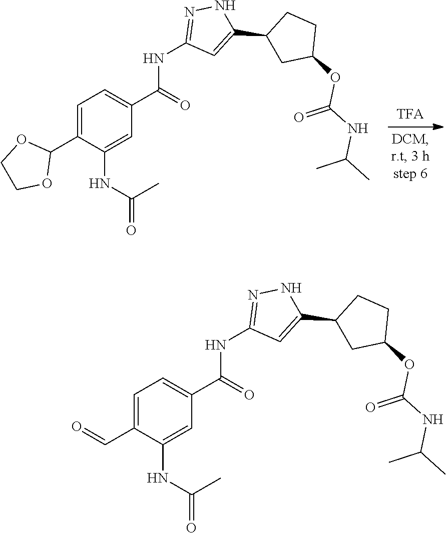 [SMILES: CC(=O)Nc1cc(C(=O)Nc2cc([C@H]3CC[C@@H](OC(=O)NC(C)C)C3)[nH]n2)ccc1C1OCCO1 ; CC(=O)Nc1cc(C(=O)Nc2cc([C@H]3CC[C@@H](OC(=O)NC(C)C)C3)[nH]n2)ccc1C=O]

### Step 1: Synthesis of methyl 4-(1,3-dioxolan-2-yl)-3-nitrobenzoate

A solution of methyl 4-formyl-3-nitrobenzoate (10.0 g, 47.811 mmol, 1.0 equiv) and PPTS (1.2 g, 4.781 mmol, 0.1 equiv) in toluene (50 mL) was treated with ethylene glycol (17.8 g, 286.866 mmol, 6.0 equiv) and triethyl orthoformate (21.3 g, 143.433 mmol, 3.0 equiv) under N₂ atmosphere. The resulting mixture was stirred for 12 hours at 90° C. The resulting mixture was washed with H₂O (50 mL) and extracted EA (30 mL×3). The combined organic layers were washed with saturated sodium chloride solution (50 mL), dried over anhydrous Na₂SO₄. The organic layers were concentrated under reduced pressure. The residue was purified by silica gel column chromatography, eluted with EA/PA (30%) to afford methyl 3-dinobeliumyl-4-(1,3-dioxolan-2-yl)benzoate (5.0 g, 14.42%) as a yellow solid. ¹H NMR (400 MHz, DMSO-d₆) δ 8.39 (dt, J₁=6.1, J₂=1.8 Hz, 1H), 8.27 (ddq, J₁=9.0, J₂=6.3, J₃=2.1 Hz, 1H), 7.91 (ddd, J=8.4, J₂=4.7, J₃=1.7 Hz, 1H), 6.38 (d, J=2.3 Hz, 1H), 4.03-3.97 (m, 2H), 3.96-3.94 (m, 2H), 3.92 (s, 3H).

### Step 2: Synthesis of methyl 3-amino-4-(1,3-dioxolan-2-yl)benzoate

A solution of methyl 4-(1,3-dioxolan-2-yl)-3-nitrobenzoate (2.5 g, 9.873 mmol, 1.0 equiv) in EA (20 mL) was treated with Pd/C (1.0 g, 9.397 mmol, 0.95 equiv) at rt under H₂ atmosphere. The resulting mixture was stirred for 2 hours at rt. The resulting mixture was filtered, the filter cake was washed with EA (20 mL×3). The filtrate was concentrated under reduced pressure. The residue was purified by silica gel column chromatography, eluted with PE/EA (25%) to afford methyl 3-amino-4-(1,3-dioxolan-2-yl)benzoate (1.5 g, 61.93%) as a white solid. MS (ESI) calcd. for C₁₁H₁₃NO₄, 223.08 m/z, found 224.10 [M+H]⁺.

### Step 3: Synthesis of methyl 3-acetamido-4-(1,3-dioxolan-2-yl)benzoate

Into a 25 mL vessel was added methyl 3-amino-4-(1,3-dioxolan-2-yl)benzoate (1.5 g, 6.720 mmol, 1.0 equiv) and acetic anhydride (10 mL) rt. The resulting mixture was stirred for 1 hour at 80° C. The reaction system is cooled to room temperature. The precipitated solids were collected by filtration and washed with PE (10 mL×3) to afford methyl 4-(1,3-dioxolan-2-yl)-3-acetamidobenzoate (1.5 g, 41.23%) as white solid. MS (ESI) calcd. for C₁₃H₁₅NO₅, 265.10 m/z, found 266.10 [M+H]⁺.

### Step 4: Synthesis of 3-acetamido-4-(1,3-dioxolan-2-yl)benzoic acid

To a stirred solution of methyl 4-(1,3-dioxolan-2-yl)-3-acetamidobenzoate (700 mg, 2.639 mmol, 1 equiv) in THF (10 mL) and H₂O (2 mL) was added LiOH·H₂O (199.3 mg, 4.750 mmol, 1.8 equiv) at rt and stirred for 6 hours. The reaction progress was monitored by TLC. After completion of reaction, the reaction mixture was concentrated under reduced pressure. Add 5 mL water to the system and stir at 0° C. The mixture was neutralized with HCl (2M). The resulting mixture was extracted with EA (20 mL×3). The combined organic layers were washed with saturated sodium chloride solution (30 mL), dried over anhydrous Na₂SO₄, concentrated under reduced pressure to afford 4-(1,3-dioxolan-2-yl)-3-acetamidobenzoic acid (488 mg, 51.52%) as yellow solid. MS (ESI) calcd. for C₁₂H₁₃NO₅, 251.08 m/z, found 252.10 [M+H]⁺.

### Step 5: Synthesis of (1R,3S)-3-(3-(3-acetamido-4-(1,3-dioxolan-2-yl)benzamido)-1H-pyrazol-5-yl)cyclopentyl isopropylcarbamate

To a stirred solution of 4-(1,3-dioxolan-2-yl)-3-acetamidobenzoic acid (100 mg, 0.398 mmol, 1.0 equiv) in anhydrous pyridine (5 mL) was added (1R,3S)-3-(5-amino-2H-pyrazol-3-yl)cyclopentyl N-isopropylcarbamate (130.6 mg, 0.517 mmol, 1.3 equiv) and EDCI (114.5 mg, 0.597 mmol, 1.5 equiv) at rt and stirred for 1 hour. The residue was diluted with H₂O (10 mL×5). The resulting mixture was extracted with EA (20 mL×3). The combined organic layers were washed with saturated sodium chloride solution (30 mL), dried over anhydrous Na₂SO₄, concentrated under reduced pressure. The residue was purified by Prep-TLC (MeOH/DCM=1:10) to afford (1R,3S)-3-{5-[4-(1,3-dioxolan-2-yl)-3-acetamidobenzamido]-2H-pyrazol-3-yl}cyclopentyl N-isopropylcarbamate (113 mg, 58.47%) as a yellow solid. MS (ESI) calcd. for C₂₄H₃₁N₅O₆, 485.23 m/z, found 486.25 [M+H]⁺.

### Step 6: Synthesis of (1R,3S)-3-(3-(3-acetamido-4-formylbenzamido)-1H-pyrazol-5-yl)cyclopentyl isopropylcarbamate

A solution of (1R,3S)-3-{5-[4-(1,3-dioxolan-2-yl)-3-acetamidobenzamido]-2H-pyrazol-3-yl}cyclopentyl N-isopropylcarbamate (50 mg, 0.103 mmol, 1.0 equiv) in DCM (1.5 mL) was treated with trifluoroacetaldehyde (0.5 mL) at rt and stirred for 3 hours. The resulting mixture was concentrated under vacuum. The crude product was purified by Prep-HPLC with the following conditions (Column: Xselect CSH OBD Column, 30×150 mm, 5 um; Mobile Phase A: Water (0.1% FA), Mobile Phase B: ACN; Flow rate: 60 mL/min; Gradient: 22% B to 46% B in 10 min; Wave Length: 254/220 nm; RT1 (min): 8.5) to afford (1R,3S)-3-[5-(3-acetamido-4-formylbenzamido)-2H-pyrazol-3-yl]cyclopentyl N-isopropylcarbamate (10.9 mg, 23.90%) as a white solid. MS (ESI) calcd. for C₂₂H₂₇N₅O₅, 441.20 m/z, found 442.10 [M+H]⁺. ¹H NMR (400 MHz, DMSO-d₆) δ 12.24 (s, 1H), 10.95 (s, 1H), 10.69 (s, 1H), 10.02 (s, 1H), 8.49 (s, 1H), 7.92 (d, J=8.0 Hz, 1H), 7.85 (d, J=7.9 Hz, 1H), 6.96 (d, J=7.7 Hz, 1H), 6.48 (s, 1H), 5.02 (s, 1H), 3.64-3.56 (m, 1H), 3.13-3.05 (m, 1H), 2.52 (s, 1H), 2.19 (s, 3H), 2.05 (s, 1H), 1.92-1.86 (m, 1H), 1.75 (d, J=9.5 Hz, 2H), 1.64 (s, 1H), 1.04 (d, J=6.6 Hz, 6H).

### Example 2.6: Synthesis of Compound 38: (1R,3S)-3-[5-(4-acetamido-3-formylbenzamido)-2H-pyrazol-3-yl]cyclopentyl N-isopropylcarbamate

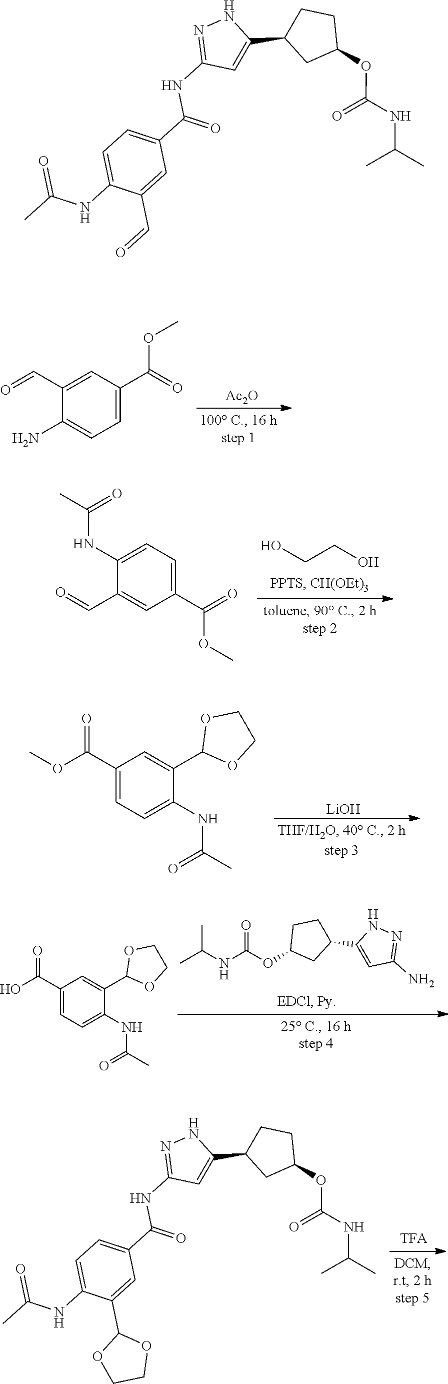

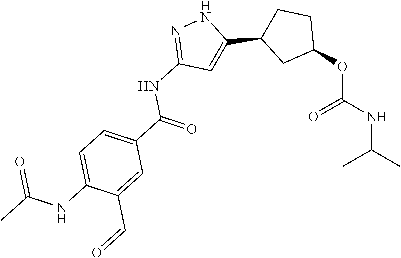 [SMILES: CC(=O)Nc1ccc(C(=O)Nc2cc([C@H]3CC[C@@H](OC(=O)NC(C)C)C3)[nH]n2)cc1C=O]

### Step 1: Synthesis of methyl 4-acetamido-3-formylbenzoate

A solution of methyl 4-amino-3-formylbenzoate (1.0 g, 5.581 mmol, 1 equiv) in Ac₂O (15 mL) was stirred for 16 h at 100° C. Desired products could be detected by LCMS. The precipitated solids were collected by filtration and washed with PE (10 mL×5). The filter cake was dried under reduced pressure to afford methyl 4-acetamido-3-formylbenzoate (1.2 g, 97.20%) as an off-white solid. MS (ESI) calcd. for C₁₁H₁₁NO₄, 221.06 m/z, found 222.10 [M+H]⁺.

### Step 2: Synthesis of methyl 3-(1,3-dioxolan-2-yl)-4-acetamidobenzoate

Into a 50 mL round bottom flask, methyl 4-acetamido-3-formylbenzoate (600.0 mg, 2.712 mmol, 1 equiv), toluene (10 mL), ethylene glycol (841.7 mg, 13.560 mmol, 5 equiv), triethyl orthoformate (1.2 g, 8.136 mmol, 3 equiv) and PPTS (68.2 mg, 0.271 mmol, 0.1 equiv) were added. The mixture was stirred for 2 h at 90° C. Desired products could be detected by LCMS. The mixture was allowed to cool down to 25° C. and was concentrated under vacuum. The resulting mixture was quench with H₂O (15 mL), extracted with EA (30 mL×3), washed with NaHCO₃ (aq. 15 mL×3). The combined organic layers were concentrated under vacuum. The residue was purified by silica gel column chromatography, eluted with PE/EA (0-50%) to afford methyl 3-(1,3-dioxolan-2-yl)-4-acetamidobenzoate (400.0 mg, 55.60%) as an off-white solid. MS (ESI) calcd. for C₁₃H₁₅NO₅, 265.09 m/z, found 266.00 [M+H]⁺.

### Step 3: Synthesis of 3-(1,3-dioxolan-2-yl)-4-acetamidobenzoic acid

A solution of methyl 3-(1,3-dioxolan-2-yl)-4-acetamidobenzoate (200.0 mg, 0.754 mmol, 1 equiv) and LiOH (25.28 mg, 1.056 mmol, 1.4 equiv) in THF (5 mL) and H₂O (1 mL) was stirred for 2 h at 40° C. Desired products could be detected by LCMS. The resulting mixture was concentrated under vacuum. The residue was dissolved in H₂O (1 mL) and acidified to pH 7 with HCl (1 mol/L) at 0° C. The resulting mixture was extracted with EA (10 mL×3), washed with brine (15 mL). The combined organic layers were concentrated under vacuum to afford the crude 3-(1,3-dioxolan-2-yl)-4-acetamidobenzoic acid (180.0 mg, 95.02%) as a yellow solid. MS (ESI) calcd. for C₁₂H₁₃NO₅, 251.07 m/z, found 252.10 [M+H]⁺.

### Step 4: Synthesis of (1R,3S)-3-{5-[3-(1,3-dioxolan-2-yl)-4-acetamidobenzamido]-2H-pyrazol-3-yl}cyclopentyl N-isopropylcarbamate

To a 25 mL round-bottom flask, 3-(1,3-dioxolan-2-yl)-4-acetamidobenzoic acid (100.0 mg, 0.398 mmol, 1 equiv), pyridine (5 mL) were added and stirred until homogeneous. (1R,3S)-3-(5-amino-2H-pyrazol-3-yl)cyclopentyl N-isopropylcarbamate (110.5 mg, 0.438 mmol, 1.1 equiv) and EDCI (91.6 mg, 0.478 mmol, 1.2 equiv) were added and the reaction mixture was stirred at 25° C. for 16 h. The resulting mixture was quench with H₂O (40 mL), extracted with EA (30 mL×3), washed with brine (20 mL). The combined organic layers were concentrated under vacuum. The residue was purified by Prep-TLC (MeOH/DCM=15:1) to afford (1R,3S)-3-{5-[3-(1,3-dioxolan-2-yl)-4-acetamidobenzamido]-2H-pyrazol-3-yl}cyclopentyl N-isopropylcarbamate (23.0 mg, 11.90%) as a yellow solid. MS (ESI) calcd. for C₂₄H₃₁N₅O₆, 485.54 m/z, found 486.25 [M+H]⁺.

### Step 5: Synthesis of (1R,3S)-3-[5-(4-acetamido-3-formylbenzamido)-2H-pyrazol-3-yl]cyclopentyl N-isopropylcarbamate

A solution of (1R,3S)-3-{5-[3-(1,3-dioxolan-2-yl)-4-acetamidobenzamido]-2H-pyrazol-3-yl}cyclopentyl N-isopropylcarbamate (23.0 mg, 0.047 mmol, 1 equiv) in DCM (2 mL) and TFA (0.25 mL) was stirred for 2 h at 25° C. Desired products could be detected by LCMS. The resulting mixture was concentrated under vacuum. The crude product was purified by Prep-HPLC with the following conditions (Column: Xselect CSH C18 OBD Column, 30×150 mm, 5 μm; Mobile Phase A: Water (0.1% FA), Mobile Phase B: ACN; Flow rate: 60 mL/min; Gradient: 23% B to 53% B in 7 min; Wave Length: 254/220 nm; RT1 (min): 5.52) to afford (1R,3S)-3-[5-(4-acetamido-3-formylbenzamido)-2H-pyrazol-3-yl]cyclopentyl N-isopropylcarbamate (6.8 mg, 32.13%) as a white solid. MS (ESI) calcd. for C₂₂H₂₇N₅O₅, 441.20 m/z, found 442.15 [M+H]⁺. ¹H NMR (300 MHz, DMSO-d₆) δ 12.22 (s, 1H), 10.89 (d, J=17.6 Hz, 2H), 10.03 (s, 1H), 8.53 (s, 1H), 8.28 (s, 2H), 6.95 (s, 1H), 6.47 (s, 1H), 5.02 (s, 1H), 3.58 (s, 1H), 3.09 (s, 1H), 2.21 (d, J=2.2 Hz, 3H), 2.02 (s, 1H), 1.86 (s, 1H), 1.74 (s, 4H), 1.04 (d, J=6.5 Hz, 6H).

### Example 2.7: Synthesis of Compound 39: (1R,3S)-3-[5-(7-formyl-1H-indazole-4-amido)-2H-pyrazol-3-yl]cyclopentyl N-isopropylcarbamate

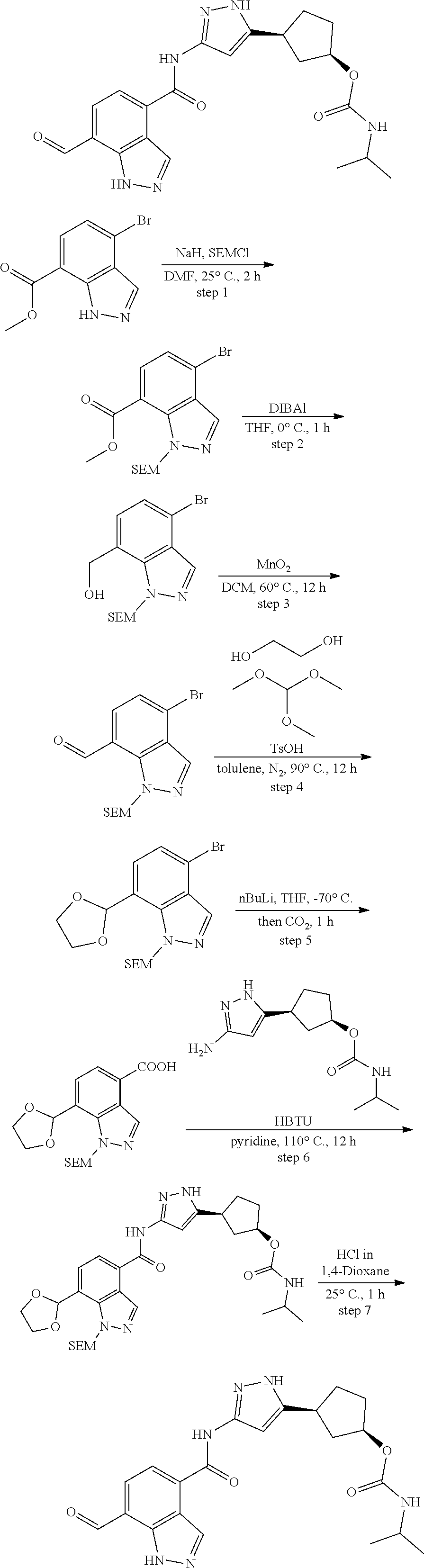

### Step 1: methyl 4-bromo-1-{[2-(trimethylsilyl)ethoxy]methyl}indazole-7-carboxylate

A solution of methyl 4-bromo-1H-indazole-7-carboxylate (2 g, 7.841 mmol, 1 equiv) in DMF (40 mL) was treated with NaH (0.5 g, 11.761 mmol, 1.5 equiv, 60%) for 0.5 h at 0° C. under nitrogen atmosphere followed by the addition of [2-(chloromethoxy)ethyl]trimethylsilane (1.9 g, 11.761 mmol, 1.5 equiv) dropwise at 0° C. The resulting mixture was stirred for 2 h at 25° C. under N₂ atmosphere. The reaction was quenched by the addition of NH₄Cl (30 mL) at 0° C. The resulting mixture was diluted with EA (30 mL) and washed with H₂O (20 mL×3), dried over anhydrous Na₂SO₄. After filtration, the filtrate was concentrated under reduced pressure. The residue was purified by silica gel column chromatography, eluted with PE/EA (50%) to afford methyl 4-bromo-1-{[2-(trimethylsilyl)ethoxy]methyl}indazole-7-carboxylate (1.5 g, 39.15%) as a yellow oil. MS (ESI) calcd. for C₁₅H₂₁BrN₂O₃Si, 384.05 m/z, found 387.05 [M+2+H]⁺.

### Step 2: (4-bromo-1-{[2-(trimethylsilyl)ethoxy]methyl}indazol-7-yl)methanol

A solution of methyl 4-bromo-1-{[2-(trimethylsilyl)ethoxy]methyl}indazole-7-carboxylate (1.5 g, 3.893 mmol, 1 equiv) in THF (20 mL) was allowed to stir for 5 min and was cooled to 0° C. under nitrogen atmosphere followed by the dropwise addition of DIBAl (1.3 mL, 7.786 mmol, 2 equiv) dropwise at 0° C. The resulting mixture was stirred for 1 h at 0° C. under N₂ atmosphere. The reaction was quenched by the addition of NH₄Cl (30 mL) at 0° C. The resulting mixture was diluted with EA (30 mL) and washed with H₂O (20 mL×3), dried over anhydrous Na₂SO₄. After filtration, the filtrate was concentrated under reduced pressure. The residue was purified by silica gel column chromatography, eluted with PE/EA (50%) to afford (4-bromo-1-{[2-(trimethylsilyl)ethoxy]methyl}indazol-7-yl)methanol (1.2 g, 73.33%) as a yellow solid. MS (ESI) calcd. for C₁₄H₂₁BrN₂O₂Si, 356.06 m/z, found 359.00 [M+H]⁺.

### Step 3: 4-bromo-1-{[2-(trimethylsilyl)ethoxy]methyl}indazole-7-carbaldehyde

A solution of (4-bromo-1-{[2-(trimethylsilyl)ethoxy]methyl}indazol-7-yl)methanol (900 mg, 2.519 mmol, 1 equiv) and MnO₂ (4.38 g, 50.380 mmol, 20 equiv) in DCM (20 mL) was stirred at 60° C. for 12 h. The resulting mixture was filtered, the filter cake was washed with DCM (30 mL×5). The filtrate was concentrated under reduced pressure. The residue was purified by silica gel column with PE/EA (0-70%) to afford 4-bromo-1-{[2-(trimethylsilyl) ethoxy]methyl}indazole-7-carbaldehyde (800 mg, 75.98%) as a yellow solid. MS (ESI) calcd. for C₁₄H₁₉BrN₂O₂Si, 354.04 m/z, found 357.00 [M+2+H]⁺.

### Step 4: 4-bromo-7-(1,3-dioxolan-2-yl)-1-{[2-(trimethylsilyl)ethoxy]methyl}indazole

A solution of 4-bromo-1-{[2-(trimethylsilyl)ethoxy]methyl}indazole-7-carbaldehyde (800 mg, 2.252 mmol, 1 equiv), TsOH (38.8 mg, 0.225 mmol, 0.1 equiv), ethylene glycol (838.5 mg, 13.512 mmol, 6 equiv) and triethyl orthoformate (1.0 g, 6.756 mmol, 3 equiv) in toluene (10 mL) was stirred at 90° C. for 12 h under N₂ atmosphere. The resulting mixture was diluted with EA (50 mL) and washed with H₂O (30 mL×2), dried over anhydrous Na₂SO₄. After filtration, the filtrate was concentrated under reduced pressure. The residue was purified by silica gel column with PE/EA (0-70%) to afford 4-bromo-7-(1,3-dioxolan-2-yl)-1-{[2-(trimethylsilyl)ethoxy]methyl}indazole (550 mg, 61.17%) as a yellow solid. MS (ESI) calcd. for C₁₆H₂₃BrN₂O₃Si, 398.07 m/z, found 398.95 [M+H]⁺.

### Step 5: 7-(1,3-dioxolan-2-yl)-1-{[2-(trimethylsilyl)ethoxy]methyl}indazole-4-carboxylic acid

A solution of 4-bromo-7-(1,3-dioxolan-2-yl)-1-{[2-(trimethylsilyl)ethoxy]methyl}indazole (250 mg, 0.626 mmol, 1 equiv) in THF (10 mL) was treated with nBuLi (0.3 mL, 0.751 mmol, 1.2 equiv) for 30 mins at −78° C. under nitrogen atmosphere followed by the addition of CO₂ (gas). The resulting mixture was stirred for 1 h at −78° C. The reaction was quenched with a saturated solution NaHCO₃ at 0° C. The resulting mixture was diluted with EA (50 mL) and washed with H₂O (30 mL×2), dried over anhydrous Na₂SO₄. After filtration, the filtrate was concentrated under reduced pressure. The residue was purified by silica gel column with PE/EA (0-70%) to afford 7-(1,3-dioxolan-2-yl)-1-{[2-(trimethylsilyl)ethoxy]methyl}indazole-4-carboxylic acid (80 mg, 31.56%) as a yellow solid. MS (ESI) calcd. for C₁₇H₂₄N₂O₅Si, 364.15 m/z, found 365.10 [M+H]⁺.

### Step 6: (1R,3S)-3-{5-[7-(1,3-dioxolan-2-yl)-1-{[2-(trimethylsilyl)ethoxy]methyl}indazole-4-amido]-2H-pyrazol-3-yl}cyclopentyl N-isopropylcarbamate

A solution of 7-(1,3-dioxolan-2-yl)-1-{[2-(trimethylsilyl)ethoxy]methyl}indazole-4-carboxylic acid (50 mg, 0.137 mmol, 1.3 equiv), (1R,3S)-3-(5-amino-2H-pyrazol-3-yl)cyclopentyl N-isopropylcarbamate (26.6 mg, 0.105 mmol, 1 equiv) and HBTU (60.0 mg, 0.158 mmol, 1.5 equiv) in pyridine (5 mL) was stirred at 110° C. for 12 h. The resulting mixture was diluted with EA (50 mL) and washed with H₂O (30 mL×2), dried over anhydrous Na₂SO₄. After filtration, the filtrate was concentrated under reduced pressure. The residue was purified by silica gel column with PE/EA (0-70%) to afford (1R,3S)-3-{5-[7-(1,3-dioxolan-2-yl)-1-{[2-(trimethylsilyl) ethoxy]methyl}indazole-4-amido]-2H-pyrazol-3-yl}cyclopentyl N-isopropylcarbamate (25 mg, 34.82%) as a yellow solid. MS (ESI) calcd. for C₂₉H₄₂N₆O₆Si, 598.29 m/z, found 599.35 [M+H]⁺.

### Step 7: (1R,3S)-3-[5-(7-formyl-1H-indazole-4-amido)-2H-pyrazol-3-yl]cyclopentyl N-isopropylcarbamate

A solution of (1R,3S)-3-{5-[7-(1,3-dioxolan-2-yl)-1-{[2-(trimethylsilyl)ethoxy]methyl}indazole-4-amido]-2H-pyrazol-3-yl}cyclopentyl N-isopropylcarbamate (25 mg, 0.042 mmol, 1 equiv) in 4 M HCl in dioxane (1 mL) was stirred at 25° C. for 0.5 h. Then the crude product was further purified by Prep-HPLC with the following condition: Column: YMC Triart C18 ExRs, 30×150 mm, 5 μm; Mobile Phase A: Water (10 mmol/L NH₄HCO₃), Mobile Phase B: ACN; Flow rate: 60 mL/min; Gradient: 18% B to 40% B in 10 min; Wave Length: 254/220 nm; RT1 (min): 9.4, it was afforded (1R,3S)-3-[5-(7-formyl-1H-indazole-4-amido)-2H-pyrazol-3-yl]cyclopentyl N-isopropylcarbamate (3 mg, 16.45%) as a light yellow solid. MS (ESI) calcd. for C₂₁H₂₄N₆O₄, 424.46 m/z, found 425.20 [M+H]⁺. ¹H NMR (300 MHz, DMSO-d₆) δ (ppm): 13.81 (s, 1H), 12.27 (s, 1H), 11.07 (s, 1H), 10.28 (s, 1H), 8.49 (s, 1H), 8.15 (d, J=7.5 Hz, 1H), 7.92 (d, J=7.4 Hz, 1H), 6.98 (s, 1H), 6.56 (s, 1H), 5.03 (s, 1H), 3.60 (d, J=7.2 Hz, 1H), 3.12 (s, 1H), 2.07 (s, 1H), 1.93 (s, 1H), 1.78 (s, 3H), 1.24 (s, 1H), 1.05 (d, J=6.5 Hz, 6H).

### Example 2.8: Synthesis of Compound 40: (1R,3S)-3-(3-(7-formyl-1H-indazole-5-carboxamido)-1H-pyrazol-5-yl)cyclopentyl isopropylcarbamate

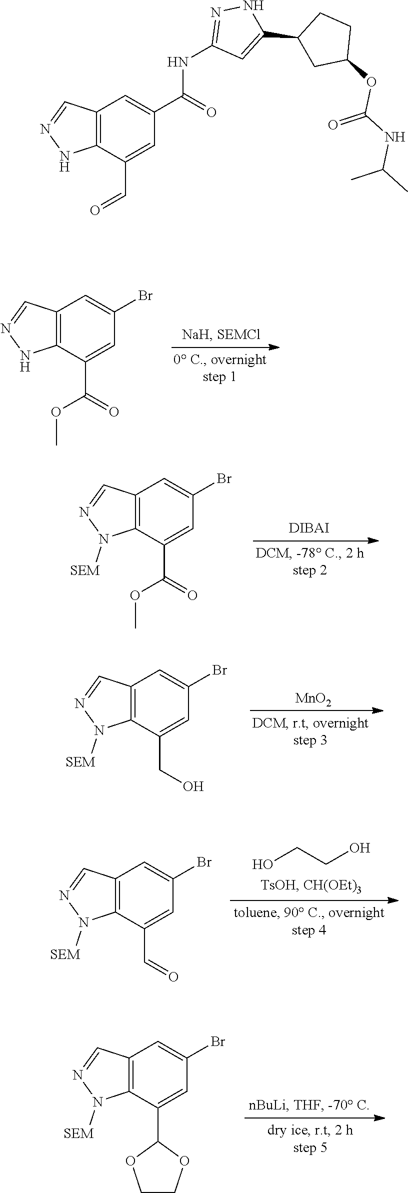

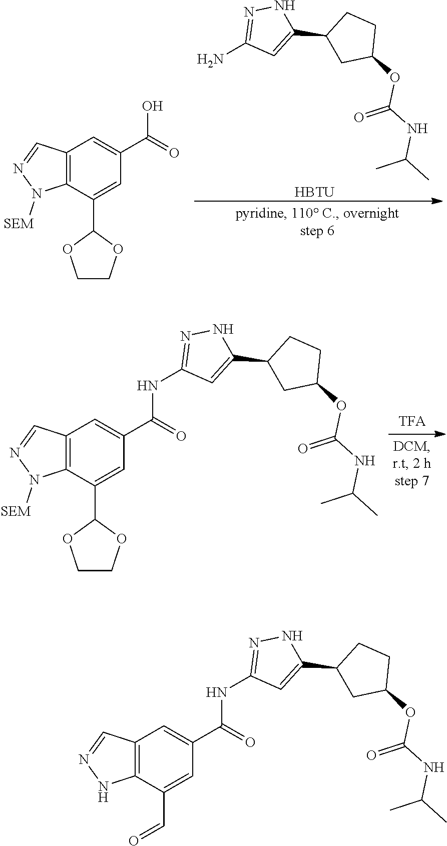 [SMILES: C[Si](C)(C)CCOCn1ncc2cc(C(=O)O)cc(C3OCCO3)c21 ; CC(C)NC(=O)O[C@@H]1CC[C@H](c2cc(N)n[nH]2)C1 ; "HBTU" ; CC(C)NC(=O)O[C@@H]1CC[C@H](c2cc(NC(=O)c3cc(C4OCCO4)c4c(cnn4COCC[Si](C)(C)C)c3)n[nH]2)C1 ; CC(C)NC(=O)O[C@@H]1CC[C@H](c2cc(NC(=O)c3cc(C=O)c4[nH]ncc4c3)n[nH]2)C1]

### Step 1: Synthesis of methyl 5-bromo-1-((2-(trimethylsilyl)ethoxy)methyl)-1H-indazole-7-carboxylate

Methyl 5-bromo-1H-indazole-7-carboxylate (5.0 g, 19.602 mmol, 1 equiv) and NaH (60%, 564.5 mg, 23.523 mmol, 1.20 equiv) were dissolved in DMF (30 mL) and stirred for 1 h at 0° C. under nitrogen atmosphere. Following this, [2-(chloromethoxy)ethyl]trimethylsilane (450.2 mg, 25.483 mmol, 1.3 equiv) was added to the mixture and stirred overnight at rt under nitrogen atmosphere. The reaction mixture was then treated with NH₄Cl (60 mL) and extracted with EA (30 mL×3), and the combined extracts washed with brine (30 mL), dried over anhydrous Na₂SO₄, filtered, and concentrated to dryness under vacuum. The residue was purified by silica gel column chromatography, eluted with PE/EA (0-10%) to afford methyl 5-bromo-1-{[2-(trimethylsilyl)ethoxy]methyl}indazole-7-carboxylate (4.8 g, 63.55%) as a yellow oil. MS (ESI) calcd. For C₁₅H₂₁BrN₂O₃Si, 384.05 m/z, found 385.10 [M+H]⁺.

### Step 2: Synthesis of (5-bromo-1-((2-(trimethylsilyl)ethoxy)methyl)-1H-indazol-7-yl)methanol

Methyl 5-bromo-1-{[2-(trimethylsilyl)ethoxy]methyl}indazole-7-carboxylate (4.8 g, 12.457 mmol, 1 equiv) was dissolved in THF (30 mL), cooled to −78° C., followed by the addition DIBAl (9.9 mL, 24.925 mmol, 2.00 equiv, 2.5 M in hexane) and allowed to stir for 2 h at −78° C. under nitrogen atmosphere. The reaction mixture was then treated with NH₄Cl (60 mL) and extracted with DCM (30 mL×3). The combined extracts were washed with brine (30 mL), dried over anhydrous Na₂SO₄, filtered, and concentrated to dryness under vacuum. The residue was purified by silica gel column chromatography, eluted with PE/EA (0-30%) to afford (5-bromo-1-{[2-(trimethylsilyl)ethoxy]methyl}indazol-7-yl)methanol (2.3 g, 51.67%) as a yellow solid. MS (ESI) calcd. For C₁₄H₂₁BrN₂O₂Si, 356.05 m/z, found 357.10 [M+H]⁺.

### Step 3: Synthesis of 5-bromo-1-((2-(trimethylsilyl)ethoxy)methyl)-1H-indazole-7-carbaldehyde

The (5-bromo-1-{[2-(trimethylsilyl)ethoxy]methyl}indazol-7-yl)methanol (1.0 g, 2.799 mmol, 1 equiv) was dissolved in DCM (20 mL), MnO₂ (4.9 g, 55.980 mmol, 20 equiv) was added to the mixture and stirred overnight at rt. Filtered, the reaction mixture was then treated with H₂O (40 mL) and extracted with DCM (30 mL×3), and the combined extracts washed with brine (20 mL), dried over anhydrous Na₂SO₄, filtered, and concentrated to dryness under vacuum. The residue was purified by silica gel column chromatography, eluted with PE/EA=(0-30%) to afford 5-bromo-1-{[2-(trimethylsilyl)ethoxy]methyl}indazole-7-carbaldehyde (450.0 mg, 45.26%) as a yellow oil. MS (ESI) calcd. For C₁₄H₁₉BrN₂O₂Si, 354.03 m/z, found 355.10 [M+H]⁺.

### Step 4: Synthesis of 5-bromo-7-(1,3-dioxolan-2-yl)-1-((2-(trimethylsilyl)ethoxy)methyl)-1H-indazole

A solution of 5-bromo-1-{[2-(trimethylsilyl)ethoxy]methyl}indazole-7-carbaldehyde (300.0 mg, 0.844 mmol, 1 equiv) in toluene (9 mL) and THF (0.4 mL) was treated with para-toluene sulfonate (14.5 mg, 0.084 mmol, 0.1 equiv) and ethylene glycol (260.0 mg, 5.064 mmol, 6 equiv), and stirred overnight at 90° C. under nitrogen atmosphere. The reaction mixture was then diluted with H₂O (30 mL) and extracted with EA (10 mL×2), the combined extracts were washed with brine (10 mL), dried over anhydrous Na₂SO₄, filtered, and concentrated to dryness under vacuum. The residue was purified by silica gel column chromatography, eluted with PE/EA (0-30%) to afford 5-bromo-7-(1,3-dioxolan-2-yl)-1-{[2-(trimethylsilyl)ethoxy]methyl}indazole (150.0 mg, 44.48%) as a pink oil. MS (ESI) calcd. For C₁₆H₂₃BrN₂O₃Si, 398.07 m/z, found 399.10 [M+H]⁺.

### Step 5: Synthesis of 7-(1,3-dioxolan-2-yl)-1-((2-(trimethylsilyl)ethoxy)methyl)-1H-indazole-5-carboxylic acid

5-Bromo-7-(1,3-dioxolan-2-yl)-1-{[2-(trimethylsilyl)ethoxy]methyl}indazole (130.0 mg, 0.326 mmol, 1 equiv) dissolved in THF (10 mL) was cooled to −78° C. and n-butyllithium (0.13 mL, 0.326 mmol, 1.00 equiv, 2.5 M in hexane) was added dropwise were and stirred for 30 mins under nitrogen atmosphere at −78° C. Dry ice (30 mg) was then added and stirred for 2 h at rt. The reaction mixture was then treated with a saturated solution of NaHCO₃ (30 mL) and extracted with DCM (10 mL×2), and the combined extracts were washed with brine (10 mL), dried over anhydrous Na₂SO₄, filtered, and concentrated to dryness under vacuum. The residue was purified by silica gel column chromatography, eluted with MeOH/DCM (0-30%). This resulted in 7-(1,3-dioxolan-2-yl)-1-{[2-(trimethylsilyl)ethoxy]methyl}indazole-5-carboxylic acid (80.0 mg, 67.43%) as a yellow solid. MS (ESI) calcd. For C₁₇H₂₄N₂O₅Si, 364.14 m/z, found 365.10 [M+H]⁺.

### Step 6: Synthesis of (1R,3S)-3-(3-(7-(1,3-dioxolan-2-yl)-1-((2-(trimethylsilyl)ethoxy)methyl)-1H-indazole-5-carboxamido)-1H-pyrazol-5-yl)cyclopentyl isopropylcarbamate

7-(1,3-Dioxolan-2-yl)-1-{[2-(trimethylsilyl)ethoxy]methyl}indazole-5-carboxylic acid (86.7 mg, 0.238 mmol, 1.5 equiv) and (1R,3S)-3-(5-amino-2H-pyrazol-3-yl)cyclopentyl N-isopropylcarbamate (40.0 mg, 0.159 mmol, 1.00 equiv) were dissolved in pyridine (10 mL). Then treated with HBTU (60.1 mg, 0.238 mmol, 1.5 equiv) and stirred overnight at 110° C. The reaction mixture was then treated with H₂O (30 mL) and extracted with EA (10 mL×3), and the combined extracts washed with brine (5 mL), dried over anhydrous Na₂SO₄, filtered, and concentrated to dryness under vacuum. The residue was purified by silica gel column chromatography, eluted with MeOH/DCM (0-10%) to afford (1R,3S)-3-{5-[7-(1,3-dioxolan-2-yl)-1-{[2-(trimethylsilyl) ethoxy]methyl}indazole-5-amido]-2H-pyrazol-3-yl}cyclopentyl N-isopropylcarbamate (60.0 mg, 63.21%) as a yellow solid. MS (ESI) calcd. For C₂₉H₄₂N₆O₆Si, 598.29 m/z, found 599.15 [M+H]⁺.

### Step 7: Synthesis of (1R,3S)-3-(3-(7-formyl-1H-indazole-5-carboxamido)-1H-pyrazol-5-yl)cyclopentyl isopropylcarbamate

(1R,3S)-3-{5-[7-(1,3-Dioxolan-2-yl)-1-{[2-(trimethylsilyl)ethoxy]methyl}indazole-5-amido]-2H-pyrazol-3-yl}cyclopentyl N-isopropylcarbamate (30.0 mg, 0.050 mmol, 1 equiv) was dissolved in DCM (3 mL) and TFA (1 mL) and stirred for 2 h at rt. The resulting mixture was concentrated under reduced pressure and dissolved in DMF (1 mL). The crude product was then purified by Prep-HPLC with the following conditions: Column: Xselect CSH OBD Column, 30×150 mm, 5 um; Mobile Phase A: Water (0.05% TFA), Mobile Phase B: ACN; Flow rate: 60 mL/min; Gradient: 17% B to 41% B in 9 min; Wave Length: 254 nm/220 nm; RT1 (min): 7.8. This resulted in (1R,3S)-3-[5-(7-formyl-1H-indazole-5-amido)-2H-pyrazol-3-yl]cyclopentyl N-isopropyl carbamate (2.0 mg, 9.28%) as a white solid. MS (ESI) calcd. For C₂₁H₂₄N₆O₄, 424.18 m/z, found 425.15 [M+H]⁺. 1H NMR (300 MHz, DMSO-d₆) δ (ppm): 13.90 (s, 1H), 10.93 (s, 1H), 10.25 (s, 1H), 8.86 (d, J=1.6 Hz, 1H), 8.71 (d, J=1.6 Hz, 1H), 8.42 (s, 1H), 6.98 (d, J=7.8 Hz, 1H), 6.49 (s, 1H), 5.03 (s, 1H), 3.11 (s, 3H), 2.10-1.99 (m, 3H), 1.93-1.65 (s, 3H), 1.05 (d, J=6.6 Hz, 6H).

### Example 2.9: Synthesis of Compound 62: (1R,3S)-3-(3-(7-formyl-6-hydroxy-2,3-dihydrobenzo-furan-2-carboxamido)-1H-pyrazol-5-yl)cyclopentyl isopropylcarbamate

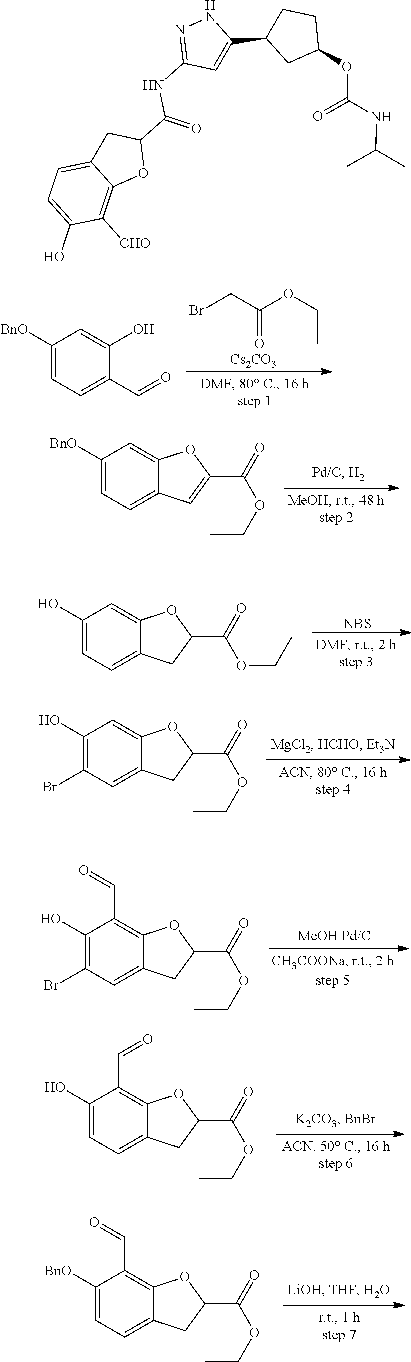

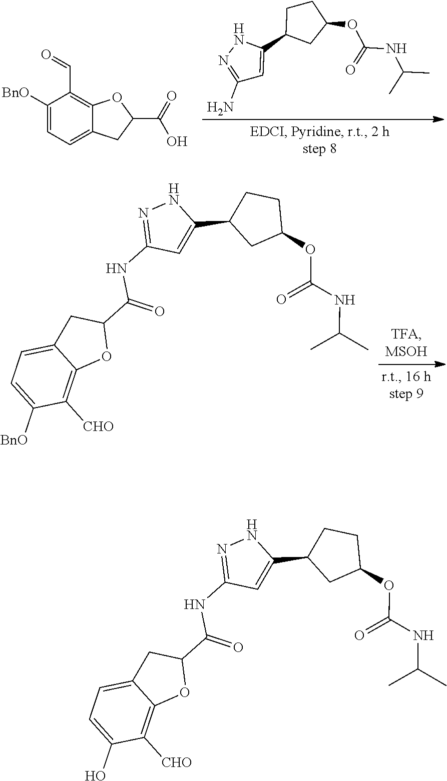 [SMILES: O=Cc1c(OCc2ccccc2)ccc2c1OC(C(=O)O)C2 ; CC(C)NC(=O)O[C@@H]1CC[C@H](c2cc(N)n[nH]2)C1 ; CC(C)NC(=O)O[C@@H]1CC[C@H](c2cc(NC(=O)C3Cc4ccc(OCc5ccccc5)c(C=O)c4O3)n[nH]2)C1 ; CC(C)NC(=O)O[C@@H]1CC[C@H](c2cc(NC(=O)C3Cc4ccc(O)c(C=O)c4O3)n[nH]2)C1]

### Step 1: Synthesis of Ethyl 6-(benzyloxy)-1-benzofuran-2-carboxylate

4-(benzyloxy)-2-hydroxybenzaldehyde (8 g, 35.050 mmol), a stir bar, cesium carbonate (22.9 g, 70.100 mmol) and dimethylformamide (90 mL) were added to a 250 mL round-bottom flask and stirred until homogeneous, then treated with ethyl bromoacetate (11.7 g, 70.100 mmol) under N₂ atmosphere. The reaction mixture was stirred at 80° C. overnight, then cooled to room temperature and diluted with water. The resulting mixture was extracted with EA (250 mL×3). The combined organic layers were washed with brine, dried over anhydrous Na₂SO₄ and filtered. After filtration, the filtrate was concentrated under reduced pressure. The residue was purified by silica gel column with PE/EA (0-50%) to afford ethyl 6-(benzyloxy)-1-benzofuran-2-carboxylate (7.8 g, 75.10%) as a white solid. MS (ESI) mass calcd. for C₁₈H₁₆O₄, 296.10 m/z, found 297.05 [M+H]⁺.

### Step 2: Synthesis of Ethyl 6-hydroxy-2,3-dihydro-1-benzofuran-2-carboxylate

Ethyl 6-(benzyloxy)-1-benzofuran-2-carboxylate (7.8 g, 26.323 mmol), a stir bar, Pd/C (7.8 g, 73.294 mmol) and methanol (250 mL) were added to a 500 mL round-bottom flask and stirred until homogeneous. The resulting mixture was stirred at room temperature for 48 h under hydrogen atmosphere, then filtered and washed with MeOH (200 mL). The filtrate was concentrated under vacuum to afford crude product. The crude product was purified by silica gel column with PE/EA (0-50%) to afford ethyl 6-hydroxy-2,3-dihydro-1-benzofuran-2-carboxylate (4.5 g, 82.11%) as a white solid. MS (ESI) mass calcd. for C₁₁H₁₂O₄, 208.07 m/z, found 209.15 [M+H]⁺.

### Step 3: Synthesis of Ethyl 5-bromo-6-hydroxy-2,3-dihydro-1-benzofuran-2-carboxylate

Ethyl 6-hydroxy-2,3-dihydro-1-benzofuran-2-carboxylate (3.4 g, 16.329 mmol), a stir bar, DMF (50 mL) were added to a 250 mL round-bottom flask and stirred until homogeneous, then treated with NBS (3.20 g, 17.962 mmol) at 0° C. The reaction mixture was stirred at rt. for 2 h, and quenched with NaHCO₃ saturated solution. The resulting mixture was extracted with EA (250 mL×3). The combined organic layers were washed with brine, dried over anhydrous Na₂SO₄. After filtration, the filtrate was concentrated under reduced pressure. The residue was purified by silica gel column with PE/EA (0-30%) to afford ethyl 5-bromo-6-hydroxy-2,3-dihydro-1-benzofuran-2-carboxylate (3.8 g, 81.05%) as a white solid. MS (ESI) mass calcd. for C₁₁H₁₁BrO₄, 285.98 m/z, found 289.00 [M+H]⁺.

### Step 4: Synthesis of Ethyl 5-bromo-7-formyl-6-hydroxy-2,3-dihydro-1-benzofuran-2-carboxylate

Ethyl 5-bromo-6-hydroxy-2,3-dihydro-1-benzofuran-2-carboxylate (3.8 g, 13.235 mmol), triethylamine (6.7 g, 66.175 mmol), paraformaldehyde (7.1 g, 79.410 mmol), a stir bar, and ACN (60 mL) were added to a 250 mL round-bottom flask and stirred until homogeneous. The resulting mixture was maintained under nitrogen and stirred at 80° C. overnight. The reaction was quenched with H₂O. The pH of the aqueous phase was adjusted to pH 4 with HCl (2M), then extracted with EA. The organic layers were combined, washed with brine, dried over Na₂SO₄, filtered and concentrated. The residue obtained was purified by silica gel chromatography (PE/EA=0-30%) to afford ethyl 5-bromo-7-formyl-6-hydroxy-2,3-dihydro-1-benzofuran-2-carboxylate (214 mg, 5.13%) as a yellow oil. MS (ESI) calcd. for C₉H₆O₃, 313.97 m/z, found: 314.90 [M+H]⁺.

### Step 5: Synthesis of Ethyl 7-formyl-6-hydroxy-2,3-dihydro-1-benzofuran-2-carboxylate

Ethyl 5-bromo-7-formyl-6-hydroxy-2,3-dihydro-1-benzofuran-2-carboxylate (200 mg, 0.732 mmol), sodium acetate (74.9 mg, 0.914 mmol) and MeOH (20 mL) were added to a 100 mL round-bottom flask and stirred until homogeneous, then treated with Pd/C (12.1 mg, 0.114 mmol). The reaction mixture was stirred at rt. for 2 h under H₂ atmosphere. Then filtered and washed with EA (20 mL). The filtrate was concentrated under vacuum to afford ethyl 7-formyl-6-hydroxy-2,3-dihydro-1-benzofuran-2-carboxylate as a yellow oil (180 mg, 96.05% yield). MS (ESI) mass calcd. for C₁₂H₁₂O₅, 236.06 m/z, found 235.20 [M−H]−.

### Step 6: Synthesis of Ethyl 6-(benzyloxy)-7-formyl-2,3-dihydro-1-benzofuran-2-carboxylate

Ethyl 7-formyl-6-hydroxy-2,3-dihydro-1-benzofuran-2-carboxylate (210 mg, 0.889 mmol), a stir bar, K₂CO₃ (247.5 mg, 1.778 mmol) and ACN (15 mL) were added to a 40 mL vail and stirred until homogeneous, then treated with benzyl bromide (228.08 mg, 1.333 mmol). The reaction mixture was stirred at 50° C. overnight and quenched with NH₄Cl, extracted with EA (60 mL×3). The combined organic layers were washed with brine, dried over anhydrous sodium sulfate, filtered and concentrated under vacuum. The residue was purified by silica gel chromatography (0-30% PE/EA) to afford ethyl 6-(benzyloxy)-7-formyl-2,3-dihydro-1-benzofuran-2-carboxylate (150 mg, 51.70%) as a yellow solid (150 mg, 51.70%). MS (ESI) mass calcd. for C₁₉H₁₈O₅, 326.11 m/z, found 327.10 [M+H]+.

### Step 7: Synthesis of 6-(benzyloxy)-7-formyl-2,3-dihydro-1-benzofuran-2-carboxylic acid

Ethyl 6-(benzyloxy)-7-formyl-2,3-dihydro-1-benzofuran-2-carboxylate (130 mg, 0.398 mmol), lithium hydroxide (13 mg, 0.557 mmol), tetrahydrofuran (10 mL) and, water (2 mL) were added to a 100 mL round-bottom flask and stirred until homogeneous. The resulting mixture was stirred at rt. for 1 h. The mixture was acidified to pH 5 with HCl (2 M). The resulting mixture was extracted with EA (3×60 mL). The combined organic layers were washed with brine, dried over anhydrous sodium sulfate, filtered, and concentrated under vacuum to afford 6-(benzyloxy)-7-formyl-2,3-dihydro-1-benzofuran-2-carboxylic acid (117 mg, 98.46%) as a white solid. MS (ESI) calcd. for C₁₇H₁₄O₅, 298.08 m/z, found 299.00 [M+H]⁺.

### Step 8: Synthesis of (1R,3S)-3-{5-[6-(benzyloxy)-7-formyl-2,3-dihydro-1-benzofuran-2-amido]-2H-pyrazol-3-yl}cyclopentyl N-isopropylcarbamate

(1R,3S)-3-(5-amino-2H-pyrazol-3-yl)cyclopentyl N-isopropylcarbamate (70 mg, 0.277 mmol), EDCI (63.8 mg, 0.332 mmol) and pyridine (7 mL) were added to a 40 vail and stirred until homogeneous, then treated with (1R,3S)-3-(5-amino-2H-pyrazol-3-yl)cyclopentyl N-isopropylcarbamate (70 mg, 0.277 mmol). The reaction mixture was stirred at rt. for 2 h. The reaction was quenched with H₂O and extracted with EA. The combined organic layers were washed with brine, dried over anhydrous sodium sulfate, filtered, and concentrated under vacuum. The residue was purified by reverse-phase chromatography (5-50%, ACN/H₂O with 0.05% TFA modifier) to afford (1R,3S)-3-{5-[6-(benzyloxy)-7-formyl-2,3-dihydro-1-benzofuran-2-amido]-2H-pyrazol-3-yl}cyclopentyl N-isopropylcarbamate (16 mg, 10.83%) as a yellow solid. MS (ESI) mass calcd. for C₂₉H₃₂N₄O₆, 532.23 m/z, found 533.15 [M+H]⁺.

### Step 9: Synthesis of (1R,3S)-3-[5-(7-formyl-6-hydroxy-2,3-dihydro-1-benzofuran-2-amido)-2H-pyrazol-3-yl]cyclopentyl N-isopropylcarbamate

(1R,3S)-3-{5-[6-(benzyloxy)-7-formyl-2,3-dihydro-1-benzofuran-2-amido]-2H-pyrazol-3-yl}cyclopentyl N-isopropylcarbamate (16 mg, 0.030 mmol), methanesulfonic acid (0.4 mL) and TFA (1.2 mL) were added to a 8 mL vail and stirred until homogeneous. The reaction mixture was stirred at rt. overnight and concentrated under vacuum. The residue was purified by Prep-HPLC (Column: Xselect CSH Prep C18 OBD Column, 30×150 mm, 5 um; Mobile Phase A: Water (0.05% TFA), Mobile Phase B: ACN; Flow rate: 60 mL/min; Gradient: 28% B to 50% B in 10 min; Wave Length: 254/220 nm) to afford (1R,3S)-3-[5-(7-formyl-6-hydroxy-2,3-dihydro-1-benzofuran-2-amido)-2H-pyrazol-3-yl]cyclopentyl N-isopropylcarbamate (0.3 mg, 2.05%) as a white solid. MS (ESI) mass calcd. for C₂₂H₂₆N₄O₆, 442.18 m/z, found 443.15 [M+H]⁺. ¹H NMR (400 MHz, Methanol-d₄) δ 7.05 (d, J=8.2 Hz, 1H), 6.49-6.36 (m, 3H), 5.85-5.83 (m, 1H), 3.52-3.48 (m, 1H), 2.67-2.42 (m, 4H), 2.26-2.18 (m, 1H), 2.18-2.10 (m, 1H), 2.03-1.78 (m, 5H), 1.59-1.31 (m, 6H), 1.26 (s, 1H), 1.25-0.94 (m, 8H), 0.94-0.88 (m, 1H).

### Example 2.10: Synthesis of Compound 174: Synthesis of (1R,3S)-3-(3-((S)-4-formyl-5-hydroxy-7-methoxy-2,3-dihydro-1H-indene-2-carboxamido)-1H-pyrazol-5-yl)cyclopentyl isopropylcarbamate

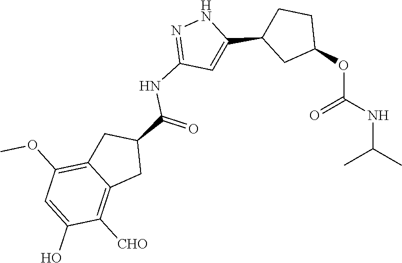 [SMILES: COc1cc(O)c(C=O)c2c1C[C@@H](C(=O)Nc1cc([C@H]3CC[C@@H](OC(=O)NC(C)C)C3)[nH]n1)C2]

### Synthetic Route

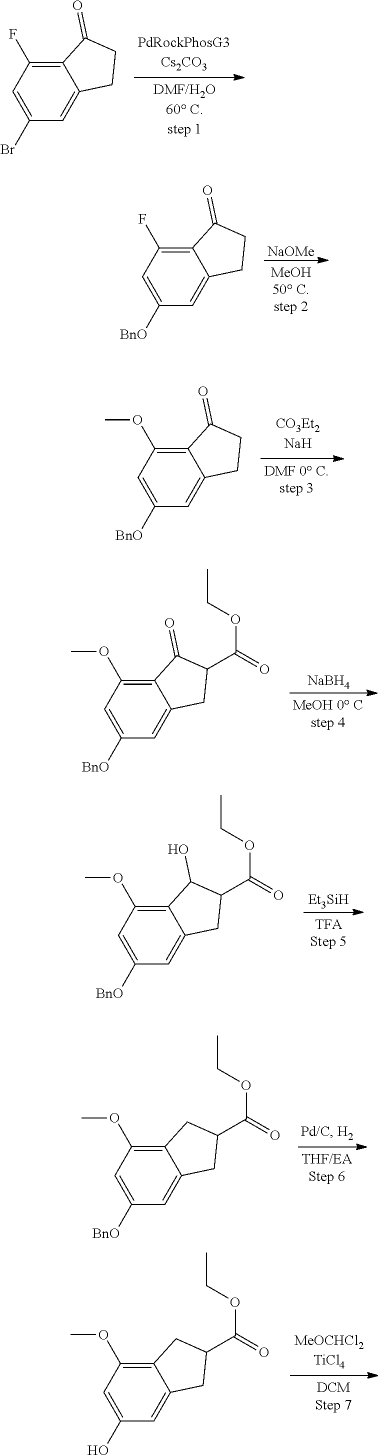

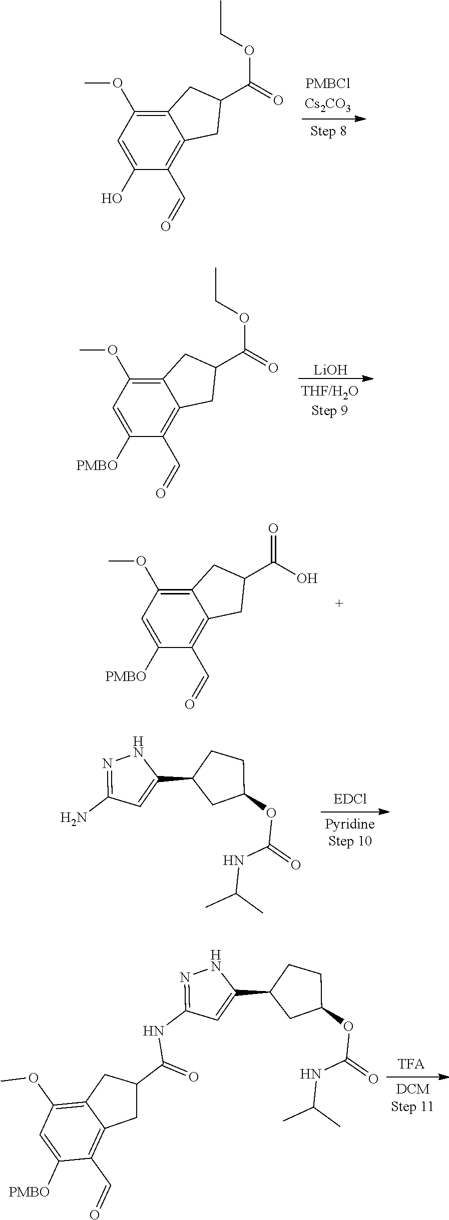

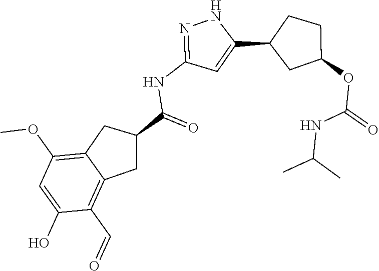 [SMILES: COc1cc(O)c(C=O)c2c1C[C@@H](C(=O)Nc1cc([C@H]3CC[C@@H](OC(=O)NC(C)C)C3)[nH]n1)C2]

### Step 1: Synthesis of 5-(benzyloxy)-7-fluoro-2,3-dihydro-1H-inden-1-one

A solution of 5-bromo-7-fluoro-2,3-dihydroinden-1-one (10.0 g, 43.659 mmol, 1.0 equiv), Cs₂CO₃ (35.6 g, 109.147 mmol, 2.5 equiv) and Rockphos Pd G3 (365.0 mg, 0.435 mmol, 0.01 equiv) in 100 mL DMF and 10 mL water was stirred at 60° C. for 1 h under nitrogen atmosphere. Following this, benzyl bromide (8.9 g, 52.386 mmol, 1.2 equiv) was added to the above mixture and stirred at room temperature for additional 1 h, the reaction was checked by LCMS and showed complete consumption of starting material. The resulting mixture was diluted with 500 mL EA. The aqueous layer was washed 3 times with 100 mL water. The combined organic extracts were dried over sodium sulphate, filtered, and concentrated to dryness. The residue was purified by silica gel column chromatography, 50-70% EA/PE to obtain 5-(benzyloxy)-7-fluoro-2,3-dihydro-1H-inden-1-one (9 g, 31.392 mmol, 72% yield). MS (ESI) calcd. for C16H13FO2, 256.09 m/z, found [M+H]⁺ 257.00 m/z.

### Step 2: Synthesis of 5-(benzyloxy)-7-methoxy-2,3-dihydro-1H-inden-1-one

A solution of 5-(benzyloxy)-7-fluoro-2,3-dihydroinden-1-one (9.0 g, 35.118 mmol, 1 equiv) and sodium methoxide 30% solution in methanol (40 mL, 740.412 mmol, 21.08 equiv) in 50 mL MeOH was stirred at 50° C. for 1 h under nitrogen atmosphere, the reaction was checked by LCMS and showed complete consumption of starting material. The resulting mixture was concentrated under reduced pressure then diluted with 300 mL EA. The mixture was washed 3 times with 100 mL water. The combined organic extracts were dried over sodium sulphate, filtered, and concentrated to dryness. The crude material was purified by silica gel column chromatography, 0-40% EA/PE to obtain 5-(benzyloxy)-7-methoxy-2,3-dihydro-1H-inden-1-one (6.1 g, 20.368 mmol, 58% yield). MS (ESI) calcd. for C₁₇H₁₆O₃, 268.11 m/z, found [M+H]⁺ 269.00 m/z.

### Step 3: Synthesis of ethyl 5-(benzyloxy)-7-methoxy-1-oxo-2,3-dihydro-1H-indene-2-carboxylate

A solution of 5-(benzyloxy)-7-methoxy-2,3-dihydroinden-1-one (6.1 g, 22.735 mmol, 1 equiv) and sodium hydride (1.3 g, 34.102 mmol, 1.5 equiv, 60%) and diethyl carbonate (4.0 g, 34.102 mmol, 1.5 equiv) in 90 mL DMF was stirred at room temperature for 1 h under nitrogen atmosphere, the reaction was checked by LCMS and showed complete consumption of starting material. The resulting mixture was quenched with 200 mL saturated NH₄Cl aqueous and extracted 3 times with 100 mL EA. The combined organic extracts were dried over sodium sulphate, filtered, and concentrated to dryness. The residue was purified by silica gel column chromatography, 60-80% EA/PE to obtain ethyl 5-(benzyloxy)-7-methoxy-1-oxo-2,3-dihydro-1H-indene-2-carboxylate (8 g, 21.143 mmol, 93% yield). MS (ESI) calcd. for C₂₀H₂₀O₅, 340.13 m/z, found [M+H]⁺ 341.00 m/z.

### Step 4: Synthesis of ethyl 5-(benzyloxy)-1-hydroxy-7-methoxy-2,3-dihydro-1H-indene-2-carboxylate

A solution of ethyl 5-(benzyloxy)-7-methoxy-1-oxo-2,3-dihydroindene-2-carboxylate (8 g, 2.938 mmol, 1.0 equiv) and NaBH₄ (0.8 g, 3.915 mmol, 3.0 equiv) in 100 mL MeOH was stirred at 0° C. for 10 mins under air atmosphere, the reaction was checked by LCMS and showed complete consumption of starting material. The resulting mixture was quenched with 200 mL NH₄Cl and extracted with DCM (100 mL×3). The combined organic extracts were dried over sodium sulphate, filtered, and concentrated to dryness. The residue was purified by silica gel column chromatography, 0-50% EA/PE to obtain ethyl 5-(benzyloxy)-1-hydroxy-7-methoxy-2,3-dihydro-1H-indene-2-carboxylate (2 g, 6.128 mmol, 24% yield). MS (ESI) calcd. for C₂₀H₂₂O₅, 342.15 m/z, found [M+H]⁺ 343.00 m/z.

### Step 5: Synthesis of ethyl 6-(benzyloxy)-4-methoxy-2,3-dihydro-1H-indene-2-carboxylate

A solution of ethyl 5-(benzyloxy)-7-methoxy-1-oxo-2,3-dihydroindene-2-carboxylate (2.0 g, 6.128 mmol, 1.0 equiv) in 50 mL TFA and 50 mL triethylsilane was stirred at room temperature for 1 h, the reaction was checked by LCMS and showed complete consumption of starting material. The resulting mixture was concentrated under reduced pressure and diluted with 100 mL EA. The mixture was washed water (100 mL×3). The combined organic extracts were separated, dried over sodium sulphate, filtered, and concentrated to dryness. The reside was purified by silica gel column chromatography, 0-40% EA/PE to provide ethyl 6-(benzyloxy)-4-methoxy-2,3-dihydro-1H-indene-2-carboxylate (600.0 mg, 1.899 mmol, 31% yield). MS (ESI) calcd. for C₂₀H₂₂O₄, 326.15 m/z, found [M+H]⁺ 327.00 m/z.

### Step 6: Synthesis of ethyl 6-hydroxy-4-methoxy-2,3-dihydro-1H-indene-2-carboxylate

A solution of ethyl 6-(benzyloxy)-4-methoxy-2,3-dihydro-1H-indene-2-carboxylate (600.0 mg, 1.838 mmol, 1.0 equiv) and Pd/C (200 mg, 10% on active carbon) in 10 mL THF and 10 mL EA was stirred at room temperature for 1 h under H₂ atmosphere, the reaction was checked by LCMS and showed complete consumption of starting material. The resulting mixture was filtered the filter cake was washed 3 times with 30 mL EA. The filtrate was concentrated under reduced pressure to afford ethyl 6-hydroxy-4-methoxy-2,3-dihydro-1H-indene-2-carboxylate (370.0 mg, 0.713 mmol, 85% yield). The crude product was used for the next step without purification. MS (ESI) calcd. for C₁₃H₁₆O₄, 236.10 m/z, found [M+H]⁺ 237.00 m/z.

### Step 7: Synthesis of ethyl 4-formyl-5-hydroxy-7-methoxy-2,3-dihydro-1H-indene-2-carboxylate

A solution of ethyl 6-hydroxy-4-methoxy-2,3-dihydro-1H-indene-2-carboxylate (370.0 mg, 1.566 mmol, 1.0 equiv) and TiCl4 (4.7 mL, 4.698 mmol, 3.0 equiv) in 5 mL DCM was stirred at 0° C. for 1 h under nitrogen atmosphere. To the above mixture was added dichloro(methoxy)methane (1.5 mL, 1.566 mmol, 1.0 equiv) and stirred at room temperature for additional 1 h, the reaction was checked by LCMS and showed complete consumption of starting material. The resulting mixture was diluted with 50 mL water and extracted with 100 mL DCM. The combined organic extracts were dried over sodium sulphate, filtered, and concentrated to dryness. The crude material was purified by silica gel column chromatography, 0-40% EA/PE to afford ethyl 4-formyl-5-hydroxy-7-methoxy-2,3-dihydro-1H-indene-2-carboxylate (230.0 mg, 0.870 mmol, 55% yield). MS (ESI) calcd. for C₁₄H₁₆O₅, 264.10 m/z, found [M+H]⁺ 265.00 m/z.

### Step 8: Synthesis of ethyl 4-formyl-7-methoxy-5-((4-methoxybenzyl)oxy)-2,3-dihydro-1H-indene-2-carboxylate

A solution of ethyl 4-formyl-5-hydroxy-7-methoxy-2,3-dihydro-1H-indene-2-carboxylate (230.0 mg, 0.919 mmol, 1.0 equiv) and Cs₂CO₃ (300.3 mg, 0.919 mmol, 1.0 equiv) in 3 mL DMF was stirred at room temperature for 5 mins under air atmosphere. To the above mixture was added 4-methoxybenzyl chloride (172.7 mg, 1.103 mmol, 1.2 equiv) and stirred for additional 1 h, the reaction was checked by LCMS and showed complete consumption of starting material. The resulting mixture was diluted with 30 mL EA and washed water (10 mL×3). The combined organic extracts were dried over sodium sulphate, filtered, and concentrated to dryness. The residue was purified by prep-TLC (PE/EA=2:1) to afford ethyl 4-formyl-7-methoxy-5-((4-methoxybenzyl)oxy)-2,3-dihydro-1H-indene-2-carboxylate (240.0 mg, 0.870 mmol, 71% yield). MS (ESI) calcd. for C₂₂H₂₄O₆, 384.16 m/z, found [M+H]⁺ 385.00 m/z.

### Step 9: Synthesis of 4-formyl-7-methoxy-5-((4-methoxybenzyl)oxy)-2,3-dihydro-1H-indene-2-carboxylic acid

A solution of ethyl 4-formyl-7-methoxy-5-[(4-methoxyphenyl) methoxy]-2,3-dihydro-1H-indene-2-carboxylate (240.0 mg, 0.216 mmol, 1.0 equiv) and LiOH (46.5 mg, 0.648 mmol, 3.0 equiv) in 6 mL THF and 1.2 mL water was stirred at room temperature for 1 h, the reaction was checked by LCMS and showed complete consumption of starting material. The mixture was acidified to pH 6 with 2 M HCl. The mixture was extracted with EA (10 mL×3), the organic phased was separated, dried over sodium sulphate, filtered, and concentrated to dryness to afford 4-formyl-7-methoxy-5-((4-methoxybenzyl)oxy)-2,3-dihydro-1H-indene-2-carboxylic acid (200.0 mg, 0.870 mmol, 95% yield). The crude product was used for next step without purification. MS (ESI) calcd. for C₂₀H₂₀O₆, 356.13 m/z, found [M+H]⁺ 357.00 m/z.

### Step 10: Synthesis of (1R,3S)-3-(3-(4-formyl-7-methoxy-5-((4-methoxybenzyl)oxy)-2,3-dihydro-1H-indene-2-carboxamido)-1H-pyrazol-5-yl)cyclopentyl isopropylcarbamate

A solution of 4-formyl-7-methoxy-5-[(4-methoxyphenyl) methoxy]-2,3-dihydro-1H-indene-2-carboxylic acid (200.0 mg, 0.477 mmol, 1.0 equiv), intermediate E (180.5 mg, 0.716 mmol, 1.5 equiv) and EDCI (137.1 mg, 0.716 mmol, 1.5 equiv) in 2 mL pyridine was stirred at room temperature for 16 h under air atmosphere. The mixture was purified by prep-HPLC, 10-50% MeCN/water with 0.1% TFA modifier to afford (1R,3S)-3-(3-(4-formyl-7-methoxy-5-((4-methoxybenzyl)oxy)-2,3-dihydro-1H-indene-2-carboxamido)-1H-pyrazol-5-yl)cyclopentyl isopropylcarbamate (100.0 mg, 0.870 mmol, 61% yield). MS (ESI) calcd. for C₃₂H₃₈N₄O₇, 590.27 m/z, found [M+H]⁺ 591.00 m/z.

### Step 11: Synthesis of (1R,3S)-3-(3-((S)-4-formyl-5-hydroxy-7-methoxy-2,3-dihydro-1H-indene-2-carboxamido)-1H-pyrazol-5-yl)cyclopentyl isopropylcarbamate

A solution of (1R,3S)-3-{5-[(2S)-4-formyl-7-methoxy-5-[(4-methoxyphenyl) methoxy]-2,3-dihydro-1H-indene-2-amido]-2H-pyrazol-3-yl}cyclopentyl N-isopropylcarbamate (100.0 mg, 0.080 mmol, 1.0 equiv) in 0.3 mL TFA and 0.9 mL DCM was stirred at room temperature for 1 h, the reaction was checked by LCMS and showed complete consumption of starting material. The resulting mixture was concentrated under reduced pressure. The residue was purified by reverse phase preparatory HPLC, 28-45% MeCN/water with 0.1% TFA modifier to afford (1R,3S)-3-(3-((S)-4-formyl-5-hydroxy-7-methoxy-2,3-dihydro-1H-indene-2-carboxamido)-1H-pyrazol-5-yl)cyclopentyl isopropylcarbamate (3.7 mg, 0.006 mmol, 7.72% yield) as a white solid. ¹H NMR (400 MHz, DMSO-D6) δ 11.23 (s, 1H), 10.45 (s, 1H), 10.06 (s, 1H), 6.95 (d, J=8.1 Hz, 1H), 6.39 (s, 1H), 6.31 (s, 1H), 4.98 (s, 1H), 3.83 (s, 3H), 3.56 (q, J=6.8 Hz, 1H), 3.52-3.36 (m, 2H), 3.33-3.23 (m, 1H), 3.10-2.89 (m, 2H), 2.88-2.78 (m, 1H), 2.44 (q, J=7.8, 6.9 Hz, 1H), 1.99 (d, J=8.3 Hz, 1H), 1.92-1.83 (m, 1H), 1.70 (d, J=7.9 Hz, 2H), 1.57 (s, 1H), 1.01 (d, J=6.5 Hz, 6H). MS (ESI) calcd. for C₂₄H₃₀N₄O₆, 470.22 m/z, found [M+H]⁺ 471.00 m/z.

### Example 3 Representative Schemes following General Scheme B

### Example 3.1: Synthesis of Compound 80: (1R,3S)-3-{5-[2-(2-formyl-3-hydroxy-4-methoxyphenoxy)acetamido]-2H-pyrazol-3-yl}cyclopentyl N-isopropylcarbamate

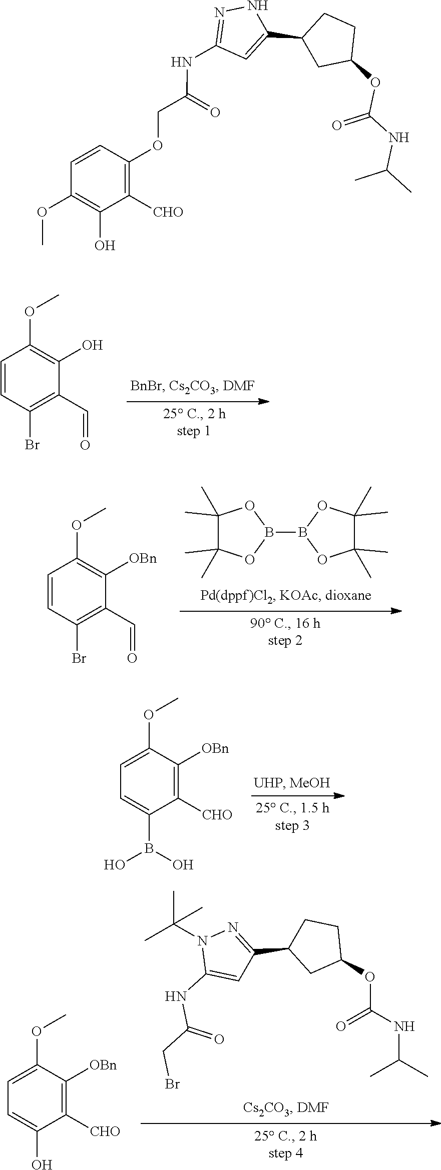

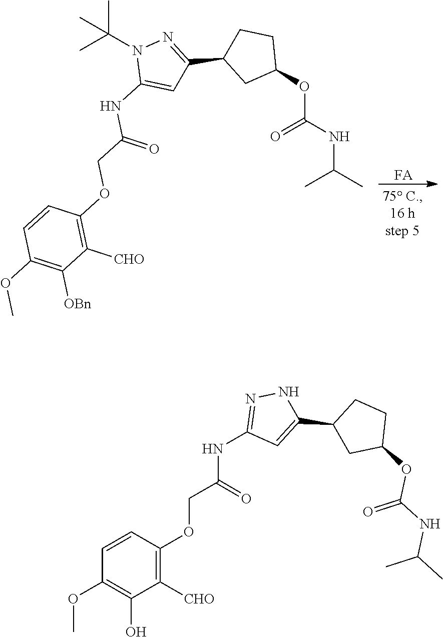

### Step 1: 2-(benzyloxy)-6-bromo-3-methoxybenzaldehyde

A solution of 6-bromo-2-hydroxy-3-methoxybenzaldehyde (2.0 g, 8.656 mmol, 1 equiv), Cs₂CO₃ (4.3 g, 12.984 mmol, 1.5 equiv) and BnBr (2.2 g, 12.984 mmol, 1.5 equiv) in DMF (25 mL) was stirred for 2 h at 25° C. Desired product could be detected by LCMS. The resulting mixture was quenched with EA (100 mL) and washed with H₂O (30 mL×2), dried over anhydrous Na₂SO₄. After filtration, the filtrate was concentrated under reduced pressure. The residue was purified by silica gel column with EA/PE (0-50%) to afford 2-(benzyloxy)-6-bromo-3-methoxy benzaldehyde (2.5 g, 83.63%) as a light yellow oil. MS (ESI) calcd. for C₁₅H₁₃BrO₃, 320.00 m/z, found 321.00 [M+H]⁺.

### Step 2: 3-(benzyloxy)-2-formyl-4-methoxyphenylboronic acid

A solution of 2-(benzyloxy)-6-bromo-3-methoxybenzaldehyde (1.3 g, 4.048 mmol, 1 equiv), bis(pinacolato)diboron (4.1 g, 16.192 mmol, 4 equiv), KOAc (1.0 g, 10.120 mmol, 2.5 equiv) and Pd(dppf)Cl₂ (0.3 g, 0.405 mmol, 0.1 equiv) in dioxane (15 mL) was stirred for 16 h at 90° C. under N₂ atmosphere. Desired product could be detected by LCMS. The resulting mixture was diluted with EA (60 mL) and washed with H₂O (10 mL×2), dried over anhydrous Na₂SO₄. The resulting mixture was concentrated under reduced pressure to afford crude 3-(benzyloxy)-2-formyl-4-methoxyphenylboronic acid (5.0 g, 43.17%) as a black oil. The crude product was used in the next step without purification. MS (ESI) calcd. for C₁₅H₁₅BO₅, 286.10 m/z, found 287.10 [M+H]⁺.

### Step 3: 2-(benzyloxy)-6-hydroxy-3-methoxybenzaldehyde

A solution of 3-(benzyloxy)-2-formyl-4-methoxyphenylboronic acid (2.3 g, 8.039 mmol, 1 equiv) and urea hydrogen peroxide (1.5 g, 16.078 mmol, 2 equiv) in MeOH (15 mL) was stirred for 1.5 h at 25° C. Desire product could be detected by LCMS. The mixture was purified by reversed-phase flash chromatography with the following conditions: column, C18 column; mobile phase, H₂O (0.5% TFA)/ACN, 50% to 70% gradient in 10 min; detector, UV 254 nm to afford 2-(benzyloxy)-6-hydroxy-3-methoxybenzaldehyde (150.0 mg, 7.01%) as a white solid. MS (ESI) calcd. for C₁₅H₁₄O₄, 258.09 m/z, found 259.00 [M+H]⁺.

### Step 4: (1R,3S)-3-(5-{2-[3-(benzyloxy)-2-formyl-4-methoxyphenoxy]acetamido}-1-tert-butylpyrazol-3-yl)cyclopentyl N-isopropylcarbamate

2-(Benzyloxy)-6-hydroxy-3-methoxybenzaldehyde (60.0 mg, 0.232 mmol, 1 equiv), Cs₂CO₃ (113.6 mg, 0.348 mmol, 1.5 equiv), KI (3.9 mg, 0.023 mmol, 0.1 equiv) and DMF (5 mL) was added to a 40 mL glass tube with a stir bar and stirred for 30 min at 25° C. (1R,3S)-3-[5-(2-bromoacetamido)-1-tert-butylpyrazol-3-yl]cyclopentyl N-isopropylcarbamate (119.6 mg, 0.278 mmol, 1.2 equiv) was added, the mixture was stirred for 2 h at 25° C. Desire product could be detected by LCMS. The resulting mixture was diluted with EA (30 mL) and washed with H₂O (8 mL×2), dried over anhydrous Na₂SO₄. After filtration, the filtrate was concentrated under reduced pressure. The residue was purified by silica gel column with EA/PE (0-60%) to afford (1R,3S)-3-(5-{2-[3-(benzyloxy)-2-formyl-4-methoxyphenoxy]acetamido}-1-tert-butylpyrazol-3-yl)cyclo pentyl N-isopropylcarbamate (70.0 mg, 42.21%) as a white solid. MS (ESI) calcd. for C₃₃H₄₂N₄O₇, 606.31 m/z, found 607.31 [M+H]⁺.

### Step 5: (1R,3S)-3-{5-[2-(2-formyl-3-hydroxy-4-methoxyphenoxy)acetamido]-2H-pyrazol-3-yl}cyclopentyl N-isopropylcarbamate

A solution of (1R,3S)-3-(5-{2-[3-(benzyloxy)-2-formyl-4-methoxyphenoxy]acetamido}-1-tert-butylpyrazol-3-yl)cyclopentyl N-isopropylcarbamate (70.0 mg, 0.115 mmol, 1 equiv) in formic acid (1.5 mL) was stirred for 16 h at 75° C. Desired product could be detected by LCMS. The residue was further purified by Prep-HPLC with Column: Xselect CSH C18 OBD Column: X select CSH Prep C18 OBD Column, 30×150 mm, 5 um; Mobile Phase A: Water (0.1% FA), Mobile Phase B: ACN; Flow rate: 60 mL/min; Gradient: 24% B to 46% B in 10 min; Wave Length: 254 nm/220 nm nm to afford (1R,3S)-3-{5-[2-(2-formyl-3-hydroxy-4-methoxyphenoxy) acetamido]-2H-pyrazol-3-yl}cyclopentyl N-isopropylcarbamate (10.7 mg, 19.29%) as a light yellow solid. MS (ESI) calcd for C₂₂H₂₈N₄O₇, 460.20 m/z, found 461.20 [M+H]⁺. ¹H NMR (300 MHz, DMSO-d₆) δ 12.15 (s, 1H), 11.73 (s, 1H), 10.48 (s, 1H), 10.39 (s, 1H), 7.23 (d, J=8.9 Hz, 1H), 6.93 (d, J=7.4 Hz, 1H), 6.43 (d, J=9.0 Hz, 1H), 6.32 (s, 1H), 4.92-5.08 (m, 1H), 4.76 (s, 2H), 3.76 (s, 3H), 3.47-3.63 (m, 1H), 2.95-3.13 (m, 1H), 2.44-2.51 (m, 1H), 1.92-2.09 (m, 1H), 1.81-1.91 (m, 1H), 1.66-1.80 (m, 2H), 1.52-1.65 (m, 1H), 1.03 (d, J=6.6 Hz, 6H).

### Example 3.2: Synthesis of Compound 100: 2-(benzyloxy)-4-(difluoromethoxy)-6-[(4-methoxyphenyl)methoxy]benzaldehyde

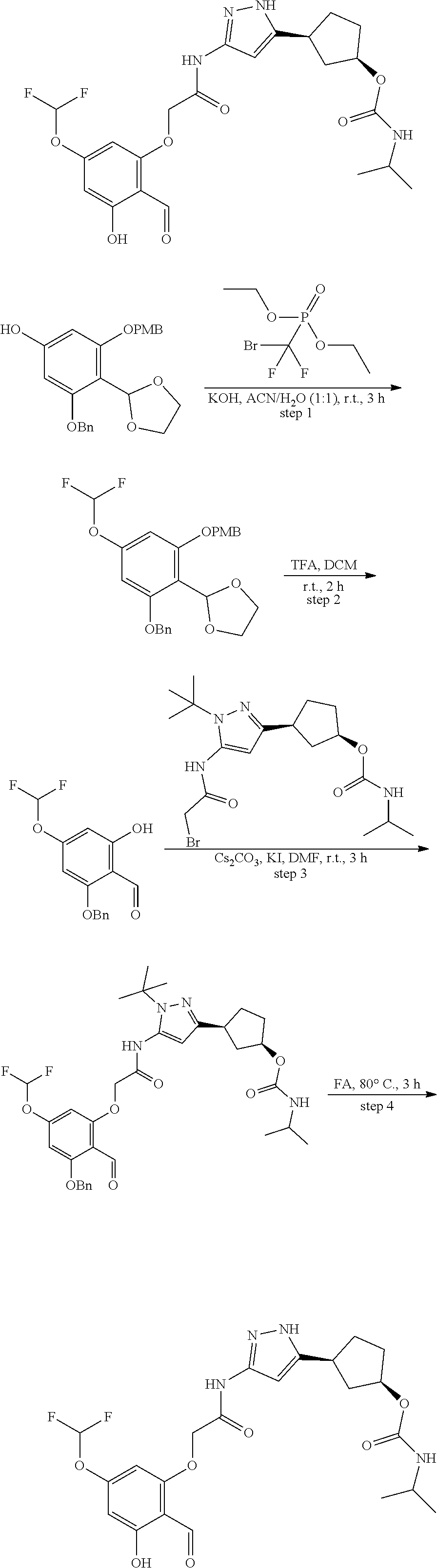

### Step 1: Synthesis of 2-(benzyloxy)-4-(difluoromethoxy)-6-[(4-methoxyphenyl)methoxy]benzaldehyde

3-(benzyloxy)-4-(1,3-dioxolan-2-yl)-5-[(4-methoxyphenyl)methoxy]phenol (400 mg, 0.979 mmol), KOH (549 mg, 9.790 mmol), CH₃CN (2.5 mL), a stir bar and H₂O (2.5 mL) were added to an oven-dried 50 mL round bottom flask and stirred until homogenous, then treated with diethyl bromodifluoromethylphosphonate (654 mg, 2.447 mmol) in batches at 0° C. The resulting mixture was stirred at rt for 3 h, then concentrated under vacuum. The residue was purified by silica gel chromatography (0-25% PE/EA) to afford 2-(benzyloxy)-4-(difluoromethoxy)-6-[(4-methoxy phenyl)methoxy]benzaldehyde (160 mg, 39.43%) as a white solid. MS (ESI) calcd. for C₂₅H₂₄F₂O₆, 458.15, found, 459 [M+H]⁺.

### Step 2: Synthesis of 2-(benzyloxy)-4-(difluoromethoxy)-6-hydroxybenzaldehyde

2-(benzyloxy)-4-(difluoromethoxy)-6-[(4-methoxyphenyl)methoxy]benzaldehyde (150 mg, 0.362 mmol), TFA (0.5 mL), a stir bar and DCM (2.5 mL) were added to an oven-dried 50 mL round bottom flask and stirred until homogenous. The resulting mixture was stirred for 2 h at rt, then concentrated under vacuum. The residue was purified by reverse-phase chromatography (0-62% ACN/H₂O with 0.05% TFA) to afford 2-(benzyloxy)-4-(difluoromethoxy)-6-hydroxybenz-aldehyde (100 mg, 93.89%) as a yellow solid. MS (ESI) calcd. for C₁₅H₁₂F₂O₄, 294.07 m/z, found, 295 m/z [M+H]⁺.

### Step 3: Synthesis of (1R,3S)-3-(5-{2-[3-(benzyloxy)-5-(difluoromethoxy)-2-formylphenoxy]acetamido}-1-tert-butylpyrazol-3-yl)cyclopentyl N-isopropylcarbamate

2-(benzyloxy)-4-(difluoromethoxy)-6-hydroxybenzaldehyde (100 mg, 0.340 mmol), (1R,3S)-3-[5-(2-bromoacetamido)-1-tert-butylpyrazol-3-yl]cyclopentyl N-isopropylcarbamate (146 mg, 0.340 mmol), Cs₂CO₃ (221 mg, 0.680 mmol), KI (5.6 mg, 0.034 mmol), a stir bar and DMF (3 mL) were added to an oven-dried 50 mL round bottom flask and stirred until homogenous. The resulting mixture was stirred at rt for 3 h, then concentrated under vacuum. The residue was purified by reverse-phase chromatography (0-67% ACN/H₂O with 0.05% TFA) to afford (1R,3S)-3-(5-{2-[3-(benzyloxy)-5-(difluoromethoxy)-2-formylphenoxy]acetamido}-1-tert-butylpyrazol-3-yl)cyclo pentyl N-isopropylcarbamate (85 mg, 38.92%) as a yellow solid. MS (ESI) calcd. for C₃₃H₄₀F₂N₄O₇, 642.29, found, 643 [M+H]⁺.

### Step 4: Synthesis of (1R,3S)-3-(5-{2-[5-(difluoromethoxy)-2-formyl-3-hydroxyphenoxy]acetamido}-2H-pyrazol-3-yl)cyclopentyl N-isopropylcarbamate

(1R,3S)-3-(5-{2-[3-(benzyloxy)-5-(difluoromethoxy)-2-formylphenoxy]acetamido}-1-tert-butylpyrazol-3-yl)cyclopentyl N-isopropylcarbamate (80 mg, 0.124 mmol), a stir bar and HCOOH (5 mL) were added to an oven-dried 50 mL round bottom flask and stirred until homogenous. The resulting mixture was stirred at 80° C. for 3 h, then concentrated under vacuum. The residue was purified by reverse-phase chromatography (0-56% ACN/H₂O with 0.05% TFA) to afford (1R,3S)-3-(5-{2-[5-(difluoromethoxy)-2-formyl-3-hydroxyphenoxy]acetamido}-2H-pyrazol-3-yl)cyclopentyl N-isopropylcarbamate (9.3 mg, 14.34%) as a white solid. MS (ESI) calcd. for C₂₂H₂₆F₂N₄O₇, 496.18 m/z, found, 497.10 m/z [M+H]⁺. ¹H NMR (400 MHz, DMSO-d₆) δ 12.10 (s, 1H), 10.55 (s, 1H), 10.27 (s, 1H), 7.45 (t, J=72.9 Hz, 1H), 6.95 (d, J=7.9 Hz, 1H), 6.37 (s, 2H), 6.31 (s, 1H), 5.76 (s, 1H), 4.99 (d, J=7.3 Hz, 1H), 4.86 (s, 2H), 3.59-3.53 (m, 1H), 3.10-3.01 (m, 1H), 2.46-2.41 (m, 1H), 2.01 (d, J=8.9 Hz, 1H), 1.93-1.82 (m, 1H), 1.70 (t, J=10.1 Hz, 2H), 1.62-1.53 (m, 1H), 1.10-0.90 (m, 6H).

### Example 3.3: Synthesis of Compound 103: (1R,3S)-3-{5-[2-(2-formyl-3,5-dimethoxyphenoxy) acetamido]-2H-pyrazol-3-yl}cyclopentyl N-isopropylcarbamate

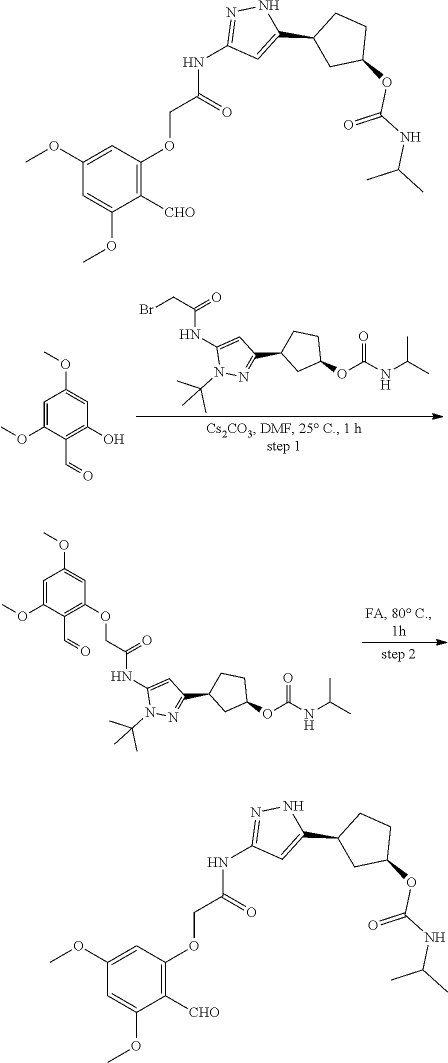

### Step 1: (1R,3S)-3-{1-tert-butyl-5-[2-(2-formyl-3,5-dimethoxyphenoxy)acetamido]pyrazol-3-yl}cyclopentyl N-isopropylcarbamate

A solution of 2-hydroxy-4,6-dimethoxybenzaldehyde (55.2 mg, 0.303 mmol, 1.3 equiv) in DMF (5 mL) was treated with Cs₂CO₃ (151.7 mg, 0.466 mmol, 2 equiv) for 0.5 h at 25° C. followed by the addition of (1R,3S)-3-[5-(2-bromoacetamido)-1-tert-butylpyrazol-3-yl]cyclopentyl N-isopropylcarbamate (100 mg, 0.233 mmol, 1.00 equiv) in portions at 25° C. The resulting mixture was stirred for 1 h at 25° C. The resulting mixture was diluted with EA (30 mL) and washed with H₂O (30 mL×2), dried over anhydrous Na₂SO₄. After filtration, the filtrate was concentrated under reduced pressure. The residue was purified by Prep-TLC (MeOH/DCM 10%) to afford (1R,3S)-3-{1-tert-butyl-5-[2-(2-formyl-3,5-dimethoxyphenoxy)acetamido]pyrazol-3-yl}cyclopentyl N-isopropylcarbamate (30 mg, 22.09%) as a yellow solid. MS (ESI) calcd. for C₂₇H₃₈N₄O₇, 530.62 m/z, found 531.35 m/z [M+H]⁺.

### Step 2: (1R,3S)-3-{5-[2-(2-formyl-3,5-dimethoxyphenoxy)acetamido]-2H-pyrazol-3-yl}cyclo-pentyl N-isopropylcarbamate

A solution of (1R,3S)-3-{1-tert-butyl-5-[2-(2-formyl-3,5-dimethoxyphenoxy)acetamido]pyrazol-3-yl}cyclopentyl N-isopropylcarbamate (30 mg, 0.094 mmol, 1 equiv) in formic acid (1 mL) was stirred at 80° C. for 1 h. Then the crude product was further purified by Prep-HPLC with the following condition: Column: Xselect CSH Fluoro Phenyl 30×150 mm, 5 μm; Mobile Phase A: Water (0.1% FA), Mobile Phase B: ACN; Flow rate: 60 mL/min; Gradient: 29% B to 45% B in 10 min; Wave Length: 254/220 nm; RT1 (min): 9.42, it was afforded (1R,3S)-3-{5-[2-(2-formyl-3,5-dimethoxyphenoxy)acetamido]-2H-pyrazol-3-yl}cyclopentyl N-isopropylcarbamate (5.7 mg, 12.66%) as a white solid. MS (ESI) calcd. for C₂₃H₃₀N₄O₇, 474.51 m/z, found 475.25 m/z [M+H]⁺. ¹H NMR (300 MHz, DMSO-d₆) δ (ppm): 12.18 (s, 1H), 10.35 (s, 1H), 10.27 (s, 1H), 6.97 (d, J=7.8 Hz, 1H), 6.33-6.22 (m, 3H), 5.00 (s, 1H), 4.76 (s, 2H), 3.88 (d, J=2.2 Hz, 6H), 3.64-3.49 (m, 2H), 3.13-3.02 (m, 1H), 2.07-1.96 (m, 1H), 1.99-1.80 (m, 1H), 1.71 (s, 2H), 1.60 (s, 1H), 1.03 (d, J=6.6 Hz, 6H).

### Example 3.4: Synthesis of Compound 108: (1s,3s)-3-(3-(2-(2-formyl-3,5-dimethoxyphenoxy)acetamido)-1H-pyrazol-5-yl)cyclobutyl 3-oxa-6-azabicyclo[3.1.1]heptane-6-carboxylate

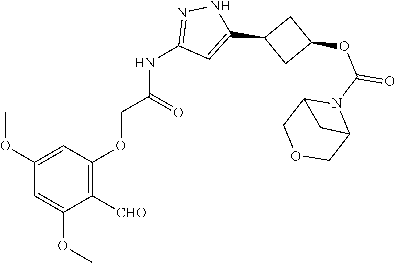 [SMILES: COc1cc(OC)c(C=O)c(OCC(=O)Nc2cc([C@H]3C[C@@H](OC(=O)N4C5COCC4C5)C3)[nH]n2)c1]

### Synthetic Route

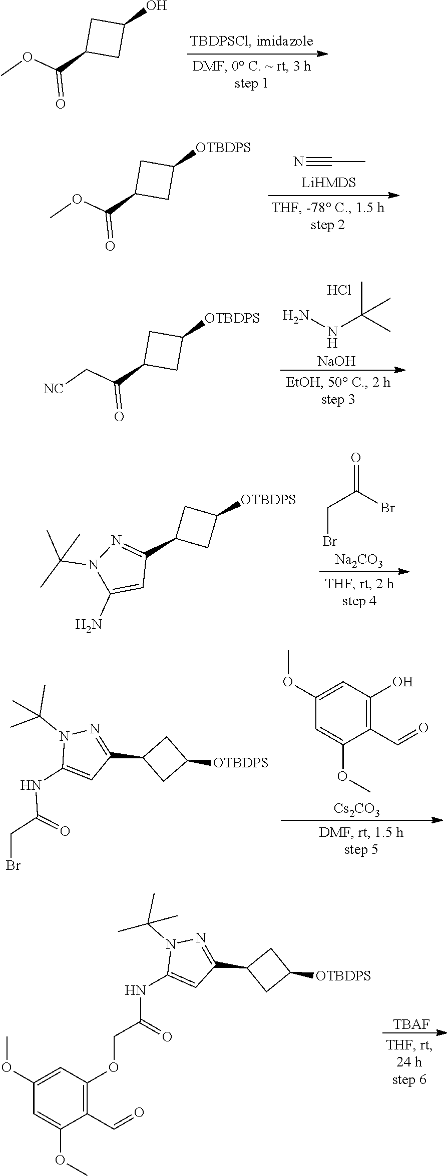

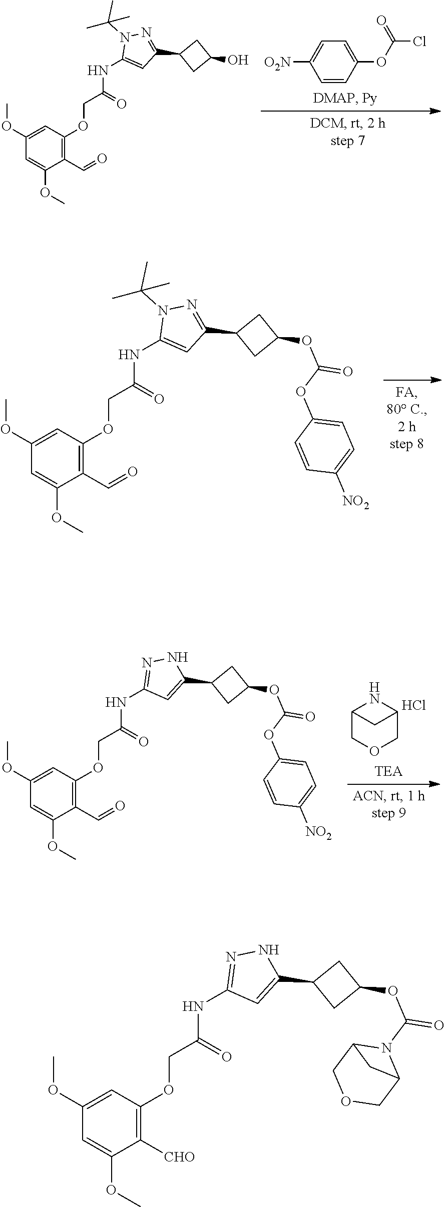

### Step 1: Synthesis of Methyl (1s,3s)-3-[(tert-butyldiphenylsilyl)oxy]cyclobutane-1-carboxylate

Methyl (1s,3s)-3-hydroxycyclobutane-1-carboxylate (20.0 g, 153.7 mmol, 1.0 equiv), a stir bar, imidazole (31.4 g, 461 mmol, 3.0 equiv) and DMF (250 mL) were added to a 1000 mL bottom-round flask and stirred until homogenous at 0° C. The mixture was then treated with tert-butylchlorodiphenylsilane (105.6 g, 384.192 mmol, 2.5 equiv) dropwise at 0° C. The resulting mixture was stirred for 3 h at rt. The resulting mixture was diluted with water and extracted with EA (3×1500 mL). The combined organic layers were washed with water, brine, and dried over anhydrous sodium sulfate. After filtration, the filtrate was concentrated under vacuum. The residue was purified by silica gel column chromatography, eluted with (0-8% PE/EA) to afford methyl (1s,3s)-3-[(tert-butyldiphenylsilyl)oxy]cyclobutane-1-carboxylate (42.0 g, 68.9%) as a colorless oil. MS (ESI) calcd. for C₂₂H₂₈O₃Si: 368.18 m/z, found: 369.20 [M+H]⁺.

### Step 2: Synthesis of 3-((1s,3s)-3-[(tert-butyldiphenylsilyl)oxy)cyclobutyl]-3-oxopropanenitrile

Methyl (1s,3s)-3-[(tert-butyldiphenylsilyl)oxy]cyclobutane-1-carboxylate (15.0 g, 40.700 mmol, 1.0 equiv), a stir bar, THF (150 mL) and acetonitrile (5.1 g, 124.230 mmol, 3.1 equiv) were added to a 500 mL round-bottom flask and stirred until homogenous at −78° C. under nitrogen atmosphere. The mixture was then treated with a solution of LiHMDS (81.5 mL, 81.5 mmol, 2.0 equiv, 1 mol/mL) dropwise at −78° C. under nitrogen atmosphere. The resulting mixture was stirred for 1.5 h at −78° C. under N₂ and quenched with a saturated solution of NH₄Cl. The resulting mixture was let warm to rt then extracted with EA (3×500 mL). The combined organic layer was dried over anhydrous sodium sulfate. After filtration, the filtrate was concentrated under vacuum to afford 3-[(1s,3s)-3-((tert-butyldiphenylsilyl)oxy)cyclobutyl]-3-oxopropanenitrile (16.2 g, crude) as a yellow oil which was used in the next step without further purification. MS (ESI) calcd. for C₂₃H₂₇NO₂Si: 377.18 m/z, found: 400.20 [M+Na]⁺.

### Step 3: Synthesis of 1-(tert-butyl)-3-[(1s,3s)-3-[(tert-butyldiphenylsilyl)oxy]cyclobutyl]-1H-pyrazol-5-amine

tert-Butylhydrazine hydrochloride (7.4 g, 59.385 mmol, 1.5 equiv), NaOH (1.6 g, 40.003 mmol, 1.0 equiv), a stir bar and EtOH (50 mL) were added to a 500 mL round-bottom flask and stirred for 0.5 h. Then 3-[(1s,3s)-3-((tert-butyldiphenylsilyl)oxy)cyclobutyl]-3-oxopropanenitrile (15.1 g, 40 mmol, 1.0 equiv) was dissolved with EtOH (100 mL), and the above mixture was added. The resulting mixture was stirred for 1.5 h at 50° C., diluted with water and extracted with EA (3×300 mL). The combined organic layer was dried over anhydrous sodium sulfate. After filtration, the filtrate was concentrated under vacuum to afford 1-(tert-butyl)-3-[(1s,3s)-3-[(tert-butyldiphenylsilyl)oxy]cyclobutyl]-1H-pyrazol-5-amine (18.2 g, crude) as a brown yellow oil which was used in the next step without further purification. MS (ESI) calcd. for C₂₇H₃₇N₃OSi: 447.27 m/z, found: 448.95 [M+H]⁺.

### Step 4: Synthesis of 2-bromo-N-(1-(tert-butyl)-3-((1s,3s)-3-((tert-butyldiphenylsilyl)oxy)cyclobutyl)-1H-pyrazol-5-yl)acetamide

1-(tert-butyl)-3-[(1s,3s)-3-[(tert-butyldiphenylsilyl)oxy]cyclobutyl]-1H-pyrazol-5-amine (28.2 g, 62.989 mmol, 1.0 equiv), Na₂CO₃ (20.1 g, 189.644 mmol, 3.0 equiv), a stir bar and THF (250 mL) were added to a 500 mL round-bottom flask and stirred until homogenous, then treated with a solution of 2-bromoacetyl bromide (25.6 g, 126.830 mmol, 2.0 equiv) dropwise at rt The resulting mixture was stirred for 2 h at rt The resulting mixture was diluted with water and extracted with DCM (3×400 mL). The combined organic layers were washed with brine, dried over anhydrous sodium sulfate. After filtration, the filtrate was concentrated under vacuum to afford 2-bromo-N-(1-(tert-butyl)-3-((1s,3s)-3-((tert-butyldiphenylsilyl)oxy)cyclobutyl)-1H-pyrazol-5-yl)acetamide (37.7 g, crude) as a light yellow oil which was used in the next step without further purification. MS (ESI) calcd. for C₂₉H₃₈BrN₃O₂Si: 567.19 m/z, found: 568.35 [M+H]⁺.

### Step 5: Synthesis of N-(1-(tert-butyl)-3-((1s,3s)-3-((tert-butyldiphenylsilyl)oxy)cyclobutyl)-1H-pyrazol-5-yl)-2-(2-formyl-3,5-dimethoxyphenoxy)acetamide

2-hydroxy-4,6-dimethoxybenzaldehyde (12 g, 65.871 mmol, 1.0 equiv), Cs₂CO₃ (43 g, 131.568 mmol, 2.0 equiv), a stir bar and DMF (250 mL) were added to a 1000 mL round-bottom flask and stirred until homogenous, then treated with a solution of 2-bromo-N-(1-(tert-butyl)-3-((1s,3s)-3-((tert-butyldiphenylsilyl)oxy)cyclobutyl)-1H-pyrazol-5-yl)acetamide (36.7 g, 64.541 mmol, 1.0 equiv) in DMF (50 mL) dropwise at rt. The resulting mixture was stirred for 1.5 h at rt The resulting mixture was diluted with water and extracted with EA (3×500 mL). The combined organic layers were washed with water by five times and brine, dried over anhydrous sodium sulfate. After filtration, the filtrate was concentrated under vacuum. The residue was purified by silica gel column chromatography, eluted with (0-72% PE/EA) to afford N-(1-(tert-butyl)-3-((1s,3s)-3-((tert-butyldiphenylsilyl)oxy)cyclobutyl)-1H-pyrazol-5-yl)-2-(2-formyl-3,5-dimethoxyphenoxy)acetamide (14.0 g, 32.4%) as a light yellow solid. MS (ESI) calcd. for C₃₈H₄₇N₃O₆Si: 669.32 m/z, found: 670.20 [M+H]⁺.

### Step 6: Synthesis of N-(1-(tert-butyl)-3-((1s,3s)-3-hydroxycyclobutyl)-1H-pyrazol-5-yl)-2-(2-formyl-3,5-dimethoxyphenoxy)acetamide

N-(1-(tert-butyl)-3-((1s,3s)-3-((tert-butyldiphenylsilyl)oxy)cyclobutyl)-1H-pyrazol-5-yl)-2-(2-formyl-3,5-dimethoxyphenoxy)acetamide (2.0 g, 2.986 mmol, 1.0 equiv), a stir bar, and THF (20 mL) were added to a 50 mL round-bottom flask and stirred until homogenous at rt Then treated with a solution of TBAF (1.8 mL, 1.8 mmol, 0.6 equiv, 1 mol/L) dropwise at rt The resulting mixture was stirred for 1 day at rt. The crude reaction mixture was concentrated under vacuum, diluted with water, and extracted with DCM (3×100 mL). The combined organic layers were washed with water times (5×), brine, and dried over anhydrous sodium sulfate. After filtration, the filtrate was concentrated under vacuum. The residue was purified by silica gel column chromatography, eluted with (0-100% PE/EA) to afford N-(1-(tert-butyl)-3-((1s,3s)-3-hydroxycyclobutyl)-1H-pyrazol-5-yl)-2-(2-formyl-3,5-dimethoxyphenoxy)acetamide (702 mg, 54.5%) as an off-white solid. MS (ESI) calcd. for C₂₂H₂₉N₃O₆: 431.21, found: 432.25 [M+H]⁺.

### Step 7: Synthesis of (1s,3s)-3-(1-(tert-butyl)-5-(2-(2-formyl-3,5-dimethoxyphenoxy)acetamido)-1H-pyrazol-3-yl)cyclobutyl (4-nitrophenyl) carbonate

N-(1-(tert-butyl)-3-((1s,3s)-3-hydroxycyclobutyl)-1H-pyrazol-5-yl)-2-(2-formyl-3,5-dimethoxyphenoxy)acetamide (671 mg, 1.555 mmol, 1.0 equiv), 4-nitrophenyl carbonochloridate (470 mg, 2.3 mmol, 1.5 equiv), a stir bar, and DCM (6.5 mL) were added to a 40 mL vial and stirred until homogenous at rt then treated with pyridine (372 mg, 4.703 mmol, 3.0 equiv) dropwise, and DMAP (19.6 mg, 0.160 mmol, 0.1 equiv) in batches at rt. The resulting mixture was stirred for 2 h at rt, diluted with water and extracted with DCM (3×20 mL). The combined organic layers were washed with brine, dried over anhydrous sodium sulfate. After filtration, the filtrate was concentrated under vacuum. The residue was purified by silica gel column chromatography, eluted with (0-11% MeOH/DCM) to afford (1s,3s)-3-(1-(tert-butyl)-5-(2-(2-formyl-3,5-dimethoxyphenoxy)acetamido)-1H-pyrazol-3-yl)cyclobutyl (4-nitrophenyl) carbonate (859 mg, 92.6%) as an off-white solid. MS (ESI) calcd. for C₂₉H₃₂N₄O₁₀: 596.21 m/z, found: 597.05 m/z [M+H]⁺.

### Step 8: Synthesis of (1s,3s)-3-(3-(2-(2-formyl-3,5-dimethoxyphenoxy)acetamido)-1H-pyrazol-5-yl)cyclobutyl (4-nitrophenyl) carbonate

(1s,3s)-3-(1-(tert-butyl)-5-(2-(2-formyl-3,5-dimethoxyphenoxy)acetamido)-1H-pyrazol-3-yl)cyclobutyl (4-nitrophenyl) carbonate (820 mg, 1.374 mmol, 1.0 equiv), a stir bar and formic acid (8 mL) were added to a 40 mL vial. The resulting mixture was stirred for 2 h at 80° C., diluted with water and extracted with DCM (3×50 mL). The combined organic layers were washed with brine, dried over anhydrous sodium sulfate. After filtration, the filtrate was concentrated under vacuum to afford (1s,3s)-3-(3-(2-(2-formyl-3,5-dimethoxyphenoxy)acetamido)-1H-pyrazol-5-yl)cyclobutyl (4-nitrophenyl) carbonate (835 mg, crude) as a brown oil which was used in the next step without further purification. MS (ESI) calcd. for C₂₅H₂₄N₄O₁₀: 540.15 m/z, found: 541.20 m/z [M+H]⁺.

### Step 9: Synthesis of (1s,3s)-3-(3-(2-(2-formyl-3,5-dimethoxyphenoxy)acetamido)-1H-pyrazol-5-yl)cyclobutyl 3-oxa-6-azabicyclo[3.1.1]heptane-6-carboxylate

3-oxa-6-azabicyclo[3.1.1]heptane hydrochloride (30 mg, 0.221 mmol, 1.2 equiv), ACN (1.5 mL), a stir bar, and TEA (75 mg, 0.741 mmol, 3.9 equiv) were added to an 8 mL vial and stirred until homogenous at rt the reaction mixture was then treated with (1s,3s)-3-(3-(2-(2-formyl-3,5-dimethoxyphenoxy)acetamido)-1H-pyrazol-5-yl)cyclobutyl (4-nitrophenyl) carbonate (104 mg, 0.192 mmol, 1.0 equiv) in batches at rt The resulting mixture was stirred for 1 h at rt, and purified by reverse-phase chromatography (0-47% ACN/Water) to afford (1s,3s)-3-(3-(2-(2-formyl-3,5-dimethoxyphenoxy)acetamido)-1H-pyrazol-5-yl)cyclobutyl 3-oxa-6-azabicyclo[3.1.1]heptane-6-carboxylate (19.39 mg, 19.3%) as a white solid. MS (ESI) calcd. For C₂₄H₂₈N₄O₈: 500.19 m/z, found: 501.25 m/z [M+H]⁺. ¹H NMR (500 MHz, DMSO-d₆) δ 12.27 (s, 1H), 10.36 (s, 1H), 10.27 (s, 1H), 6.43-6.28 (m, 3H), 4.95-4.85 (m, 1H), 4.77 (s, 2H), 4.11-4.01 (m, 4H), 3.89 (d, J=3.8 Hz, 6H), 3.77 (d, J=57.8 Hz, 2H), 3.15-3.06 (m, 1H), 2.71 (d, J=8.8 Hz, 2H), 2.59-2.52 (m, 1H), 2.17-2.07 (m, 10.0, 7.9 Hz, 2H), 1.64 (d, J=8.3 Hz, 1H).

### Example 3.5: Synthesis of Compound 109: (1s,3s)-3-(3-(2-(2-formyl-3,5-dimethoxyphenoxy)acetamido)-1H-pyrazol-5-yl)cyclobutyl 2,2-dimethylazetidine-1-carboxylate

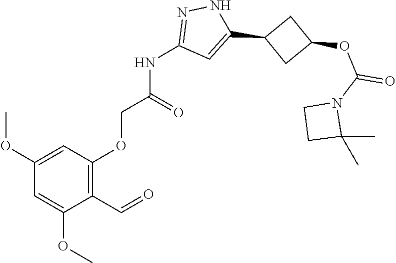 [SMILES: COc1cc(OC)c(C=O)c(OCC(=O)Nc2cc([C@H]3C[C@@H](OC(=O)N4CCC4(C)C)C3)[nH]n2)c1]

### Synthetic Route

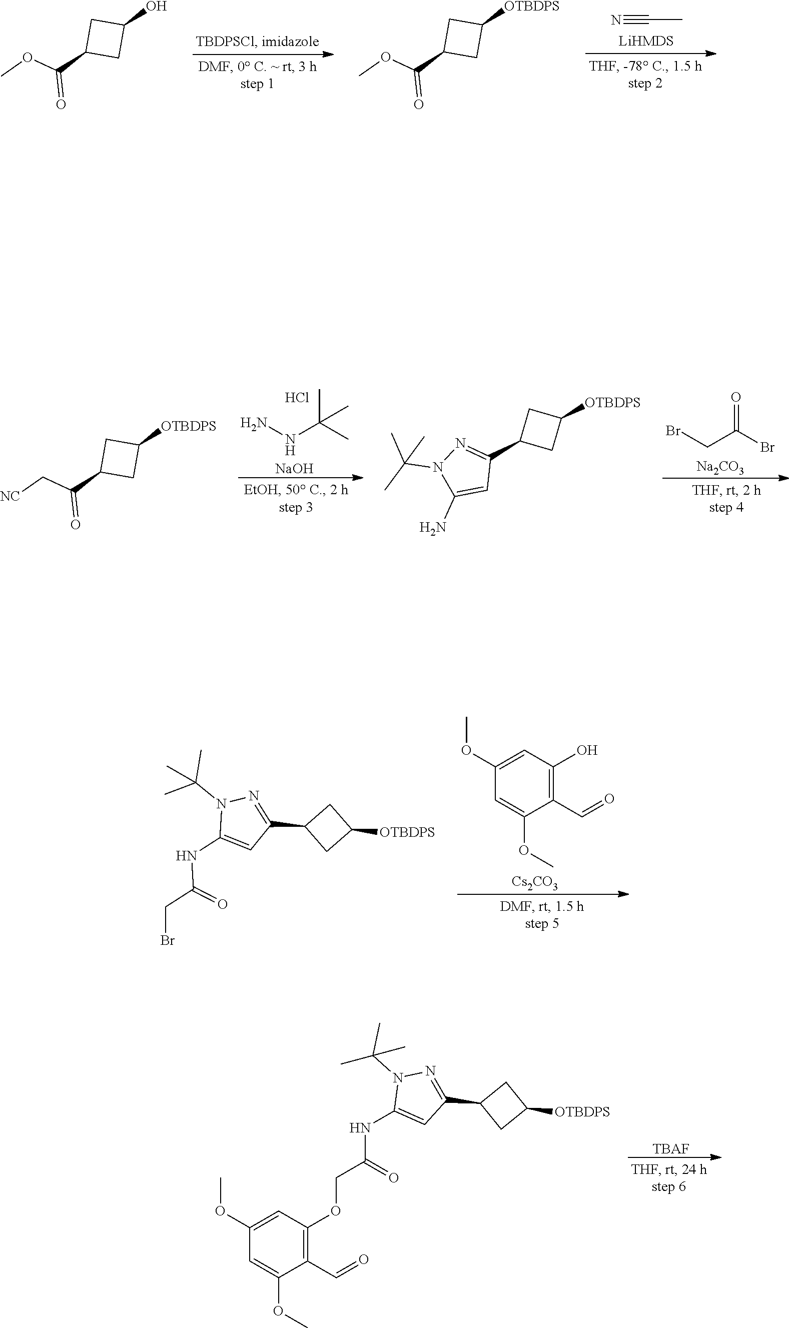

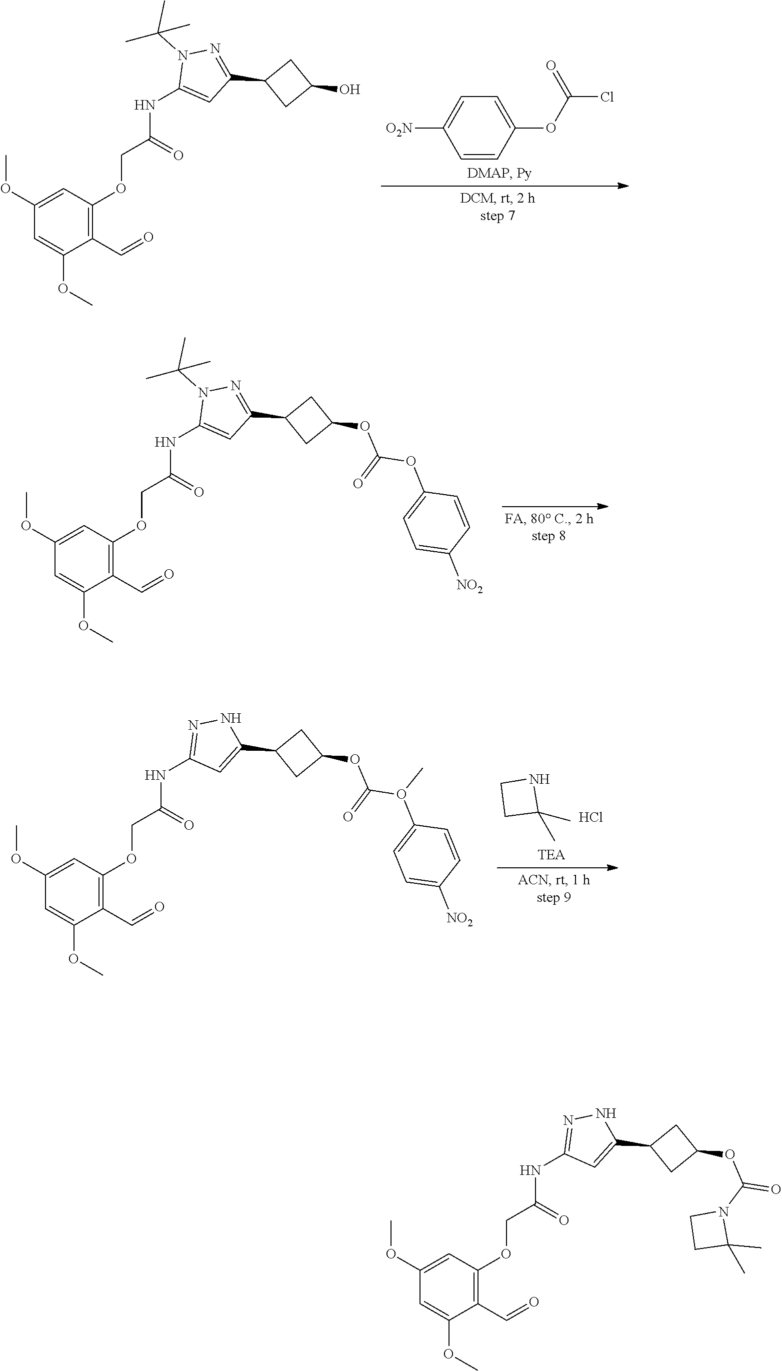

### Step 1: Synthesis of Methyl (1s,3s)-3-[(tert-butyldiphenylsilyl)oxy]cyclobutane-1-carboxylate

Methyl (1s,3s)-3-hydroxycyclobutane-1-carboxylate (20.0 g, 153.677 mmol, 1.0 equiv), a stir bar, imidazole ((31.39 g, 461.031 mmol, 3.0 equiv) and DMF (250 mL) were added to a 1000 mL bottom-round flask and stirred until homogenous at 0° C. Then treated with tert-butylchlorodiphenylsilane (105.6 g, 384.192 mmol, 2.5 equiv) dropwise at 0° C. The resulting mixture was stirred for 3 h at rt The resulting mixture was diluted with water and extracted with EA (3×1500 mL). The combined organic layers were washed with water (5×1000 mL), brine, and dried over anhydrous sodium sulfate. After filtration, the filtrate was concentrated under vacuum. The residue was purified by silica gel column chromatography, eluted with (0-8% PE/EA) to afford methyl (1s,3s)-3-[(tert-butyldiphenylsilyl)oxy]cyclobutane-1-carboxylate (42.0 g, 68.9%) as a colorless oil. MS (ESI) calcd. for C₂₂H₂₈O₃Si: 368.18 m/z, found: 369.20 [M+H]⁺.

### Step 2: Synthesis of 3-((1s,3s)-3-[(tert-butyldiphenylsilyl)oxy)cyclobutyl]-3-oxopropanenitrile

Methyl (1s,3s)-3-[(tert-butyldiphenylsilyl)oxy]cyclobutane-1-carboxylate (15.0 g, 40.700 mmol, 1.0 equiv), a stir bar, THF (150 mL) and acetonitrile (50.5 g, I₂₃0.117 mmol, 30.2 equiv) were added to a 500 mL round-bottom flask and stirred until homogenous at −78° C. under nitrogen atmosphere. Then treated with a solution of LiHMDS (81.5 mL, 81.500 mmol, 2.0 equiv, 1 mol/mL) dropwise at −78° C. under nitrogen atmosphere. The resulting mixture was stirred for 1.5 h at −78° C. under N₂ and quenched with NH₄Cl solution. The resulting mixture was let warm to rt then extracted with EA (3×500 mL). The combined organic layer was dried over anhydrous sodium sulfate. After filtration, the filtrate was concentrated under vacuum to afford 3-[(1s,3s)-3-((tert-butyldiphenylsilyl)oxy)cyclobutyl]-3-oxopropanenitrile (16.5 g, crude) as a yellow oil which was used in the next step without further purification. MS (ESI) calcd. for C₂₃H₂₇NO₂Si: 377.18 m/z, found: 400.20 [M+Na]⁺.

### Step 3: Synthesis of 1-(tert-butyl)-3-[(1s,3s)-3-[(tert-butyldiphenylsilyl)oxy]cyclobutyl]-1H-pyrazol-5-amine

tert-butylhydrazine hydrochloride (7.7 g, 61.793 mmol, 1.5 equiv), NaOH (1.76 g, 44.003 mmol, 1.1 equiv), a stir bar and EtOH (50 mL) were added to a 500 mL round-bottom flask and stirred for 0.5 h. Then 3-[(1s,3s)-3-((tert-butyldiphenylsilyl)oxy)cyclobutyl]-3-oxopropanenitrile (15.3 g, 40.523 mmol, 1.0 equiv) was dissolved with EtOH (100 mL), and the above mixture was added. The resulting mixture was stirred for 1.5 h at 50° C., diluted with water and extracted with EA (3×300 mL). The combined organic layer was dried over anhydrous sodium sulfate. After filtration, the filtrate was concentrated under vacuum to afford 1-(tert-butyl)-3-[(1s,3s)-3-[(tert-butyldiphenylsilyl)oxy]cyclobutyl]-1H-pyrazol-5-amine (18.3 g, crude) as a brown yellow oil which was used in the next step without further purification. MS (ESI) calcd. for C₂₇H₃₇N₃OSi: 447.27 m/z, found: 448.95 [M+H]⁺.

### Step 4: 2-bromo-N-(1-(tert-butyl)-3-((1s,3s)-3-((tert-butyldiphenylsilyl)oxy)cyclobutyl)-1H-pyrazol-5-yl)acetamide

1-(tert-butyl)-3-[(1s,3s)-3-[(tert-butyldiphenylsilyl)oxy]cyclobutyl]-1H-pyrazol-5-amine (28.2 g, 62.989 mmol, 1.0 equiv), Na₂CO₃ (20.1 g, 189.644 mmol, 3.0 equiv), a stir bar and THF (250 mL) were added to a 500 mL round-bottom flask and stirred until homogenous, then treated with a solution of 2-bromoacetyl bromide (25.6 g, 126.830 mmol, 2.0 equiv) dropwise at rt The resulting mixture was stirred for 2 h at rt The resulting mixture was diluted with water and extracted with DCM (3×400 mL). The combined organic layers were washed with brine, dried over anhydrous sodium sulfate. After filtration, the filtrate was concentrated under vacuum to afford 2-bromo-N-(1-(tert-butyl)-3-((1s,3s)-3-((tert-butyldiphenylsilyl)oxy)cyclobutyl)-1H-pyrazol-5-yl)acetamide (37.7 g, crude) as a light yellow oil which was used in the next step without further purification. MS (ESI) calcd. for C₂₉H₃₈BrN₃O₂Si: 567.19 m/z, found: 568.35 [M+H]⁺.

### Step 5: Synthesis of N-(1-(tert-butyl)-3-((1s,3s)-3-((tert-butyldiphenylsilyl)oxy)cyclobutyl)-1H-pyrazol-5-yl)-2-(2-formyl-3,5-dimethoxyphenoxy)acetamide

2-hydroxy-4,6-dimethoxybenzaldehyde (12 g, 65.871 mmol, 1.0 equiv), Cs₂CO₃ (43 g, 131.6 mmol, 2.0 equiv), a stir bar and DMF (250 mL) were added to a 1000 mL round-bottom flask and stirred until homogenous, then treated with a solution of 2-bromo-N-(1-(tert-butyl)-3-((1s,3s)-3-((tert-butyldiphenylsilyl)oxy)cyclobutyl)-1H-pyrazol-5-yl)acetamide (36.7 g, 64.541 mmol, 1.0 equiv) in DMF (50 mL) dropwise at rt The resulting mixture was stirred for 1.5 h at rt The resulting mixture was diluted with water and extracted with EA (3×500 mL). The combined organic layers were washed with water by five times and brine, dried over anhydrous sodium sulfate. After filtration, the filtrate was concentrated under vacuum. The residue was purified by silica gel column chromatography, eluted with (0-72% PE/EA) to afford N-(1-(tert-butyl)-3-((1s,3s)-3-((tert-butyldiphenylsilyl)oxy)cyclobutyl)-1H-pyrazol-5-yl)-2-(2-formyl-3,5-dimethoxyphenoxy)acetamide (14.0 g, 32.4%) as a light yellow solid. MS (ESI) calcd. for C₃₈H₄₇N₃O₆Si: 669.32 m/z, found: 670.20 [M+H]⁺.

### Step 6: Synthesis of N-(1-(tert-butyl)-3-((1s,3s)-3-hydroxycyclobutyl)-1H-pyrazol-5-yl)-2-(2-formyl-3,5-dimethoxyphenoxy)acetamide

N-(1-(tert-butyl)-3-((1s,3s)-3-((tert-butyldiphenylsilyl)oxy)cyclobutyl)-1H-pyrazol-5-yl)-2-(2-formyl-3,5-dimethoxyphenoxy)acetamide (2.0 g, 2.986 mmol, 1.0 equiv), a stir bar, and THF (20 mL) were added to a 50 mL round-bottom flask and stirred until homogenous at rt then treated with a solution of TBAF (1.8 mL, 1.800 mmol, 0.6 equiv, 1 mol/L) dropwise at rt. The resulting mixture was stirred for 1 day at rt. The mixture was concentrated under vacuum, diluted with water, and extracted with DCM (3×100 mL). The combined organic layers were washed with water (5×), brine, and dried over anhydrous sodium sulfate. After filtration, the filtrate was concentrated under vacuum. The residue was purified by silica gel column chromatography, eluted with (0-100% PE/EA) to afford N-(1-(tert-butyl)-3-((1s,3s)-3-hydroxycyclobutyl)-1H-pyrazol-5-yl)-2-(2-formyl-3,5-dimethoxyphenoxy)acetamide (702 mg, 54.5%) as a off-white solid. MS (ESI) calcd. for C₂₂H₂₉N₃O₆: 431.21, found: 432.25 [M+H]⁺.

### Step 7: Synthesis of (1s,3s)-3-(1-(tert-butyl)-5-(2-(2-formyl-3,5-dimethoxyphenoxy)acetamido)-1H-pyrazol-3-yl)cyclobutyl (4-nitrophenyl) carbonate

N-(1-(tert-butyl)-3-((1s,3s)-3-hydroxycyclobutyl)-1H-pyrazol-5-yl)-2-(2-formyl-3,5-dimethoxyphenoxy)acetamide (671 mg, 1.555 mmol, 1.0 equiv), 4-nitrophenyl carbonochloridate (470 mg, 2.332 mmol, 1.5 equiv), a stir bar, and DCM (6.5 mL) were added to a 40 mL vial and stirred until homogenous at rt. The resulting solution was then treated with pyridine (372 mg, 4.703 mmol, 3.0 equiv) dropwise, and DMAP (19.6 mg, 0.160 mmol, 0.1 equiv) in batches at rt. The resulting mixture was stirred for 2 h at rt, diluted with water and extracted with DCM (3×20 mL). The combined organic layers were washed with brine and dried over anhydrous sodium sulfate. After filtration, the filtrate was concentrated under vacuum. The residue was purified by silica gel column chromatography, eluted with (0-11% DCM/MEOH) to afford (1s,3s)-3-(1-(tert-butyl)-5-(2-(2-formyl-3,5-dimethoxyphenoxy)acetamido)-1H-pyrazol-3-yl)cyclobutyl (4-nitrophenyl) carbonate (859 mg, 92.6%) as an off-white solid. MS (ESI) calcd. for C₂₉H₃₂N₄O₁₀: 596.21 m/z, found: 597.05 [M+H]⁺.

### Step 8: Synthesis of (1s,3s)-3-(3-(2-(2-formyl-3,5-dimethoxyphenoxy)acetamido)-1H-pyrazol-5-yl)cyclobutyl (4-nitrophenyl) carbonate

(1s,3s)-3-(1-(tert-butyl)-5-(2-(2-formyl-3,5-dimethoxyphenoxy)acetamido)-1H-pyrazol-3-yl)cyclobutyl (4-nitrophenyl) carbonate (820 mg, 1.374 mmol, 1.0 equiv), a stir bar and formic acid (8 mL) were added to a 40 mL vial. The resulting mixture was stirred for 2 h at 80° C., diluted with water and extracted with DCM (3×50 mL). The combined organic layers were washed with brine, dried over anhydrous sodium sulfate. After filtration, the filtrate was concentrated under vacuum to afford (1s,3s)-3-(3-(2-(2-formyl-3,5-dimethoxyphenoxy)acetamido)-1H-pyrazol-5-yl)cyclobutyl (4-nitrophenyl) carbonate (835 mg, crude) as a brown oil which was used in the next step without further purification. MS (ESI) calcd. for C₂₅H₂₄N₄O₁₀: 540.15, found: 541.20 [M+H]⁺.

### Step 9: Synthesis of (1s,3s)-3-(3-(2-(2-formyl-3,5-dimethoxyphenoxy)acetamido)-1H-pyrazol-5-yl)cyclobutyl 2,2-dimethylazetidine-1-carboxylate

2,2-dimethylazetidine hydrochloride (30 mg, 0.247 mmol, 1.3 equiv), ACN (1.5 mL), a stir bar, and TEA (75 mg, 0.741 mmol, 4.0 equiv) were added to an 8 mL vial and stirred at rt until homogenous. The resulting solution was then treated with (1s,3s)-3-(3-(2-(2-formyl-3,5-dimethoxyphenoxy)acetamido)-1H-pyrazol-5-yl)cyclobutyl (4-nitrophenyl) carbonate (100 mg, 0.185 mmol, 1.0 equiv) in batches at rt. The resulting mixture was stirred for 1 h at rt, and purified by reverse-phase chromatography (0-56% ACN/Water) to afford (1s,3s)-3-(3-(2-(2-formyl-3,5-dimethoxyphenoxy)acetamido)-1H-pyrazol-5-yl)cyclobutyl 2,2-dimethylazetidine-1-carboxylate (12.9 mg, 13.9%) as an off-white solid. MS (ESI) calcd. For C₂₄H₃₀N₄O₇: 486.21 m/z, found: 487.25 [M+H]⁺. 1H NMR (500 MHz, DMSO-d₆) δ 12.26 (d, J=14.3 Hz, 1H), 10.36 (s, 1H), 10.27 (s, 1H), 6.43-6.27 (m, 3H), 4.89-4.75 (m, 3H), 3.89 (d, J=3.8 Hz, 6H), 3.81 (t, J=7.6 Hz, 1H), 3.73-3.64 (m, 1H), 3.09 (s, 1H), 2.68 (d, J=7.5 Hz, 2H), 2.13-2.03 (m, 2H), 1.98 (t, J=7.7 Hz, 2H), 1.40 (d, J=9.2 Hz, 6H)

### Example 3.6: Synthesis of Compound 113: (1s,3s)-3-(3-(2-(2-formyl-3-hydroxy-5-methoxyphenoxy)acetamido)-1H-pyrazol-5-yl)cyclobutyl isopropylcarbamate

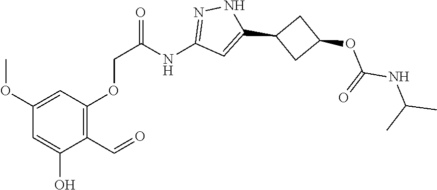 [SMILES: COc1cc(O)c(C=O)c(OCC(=O)Nc2cc([C@H]3C[C@@H](OC(=O)NC(C)C)C3)[nH]n2)c1]

### Synthetic Route

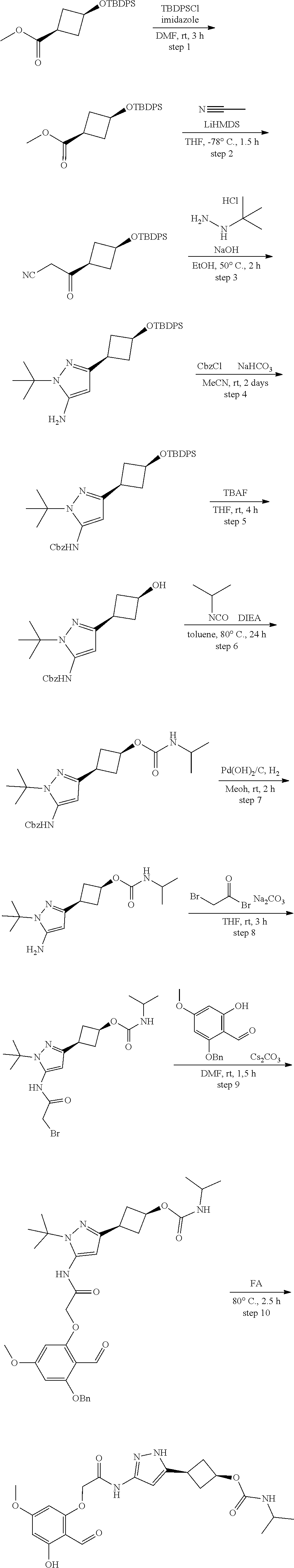

### Step 1: Synthesis of Methyl (1s,3s)-3-[(tert-butyldiphenylsilyl)oxy]cyclobutane-1-carboxylate

Methyl (1s,3s)-3-hydroxycyclobutane-1-carboxylate (2.2 g, 16.904 mmol, 1.0 equiv), a stir bar, imidazole (3.45 g, 50.676 mmol, 3.0 equiv) and DMF (20 mL) were added to a 100 mL bottom-round flask and stirred until homogenous at 0° C. The resulting solution was treated with a solution of tert-butylchlorodiphenylsilane (11.62 g, 42.276 mmol, 2.5 equiv) dropwise at 0° C. The resulting mixture was allowed to warm to rt and stir for 3 h. The mixture was diluted with water and extracted with EA (3×100 mL). The combined organic layers were washed with water (5×), brine, and dried over anhydrous sodium sulfate. After filtration, the filtrate was concentrated under vacuum. The residue was purified by silica gel column chromatography, eluted with 0-13% PE/EA to afford methyl (1s,3s)-3-[(tert-butyldiphenylsilyl)oxy]cyclobutane-1-carboxylate (6.04 g, 90.1%) as a colorless oil. MS (ESI) calcd. for C₂₂H₂₈O₃Si: 368.18 m/z, found: 369.20 [M+H]⁺.

### Step 2: Synthesis of 3-((1s,3s)-3-[(tert-butyldiphenylsilyl)oxy)cyclobutyl]-3-oxopropanenitrile

Methyl (1s,3s)-3-[(tert-butyldiphenylsilyl)oxy]cyclobutane-1-carboxylate (3.0 g, 8.140 mmol, 1.0 equiv), a stir bar, THF (30 mL) and acetonitrile (1.1 g, 26.795 mmol, 3.3 equiv) were added to a 100 mL round-bottom flask and stirred until homogenous at −78° C. under nitrogen atmosphere. The resulting solution was then treated with a solution of LiHMDS (16.2 mL, 16.200 mmol, 2.0 equiv, 1 mol/mL) dropwise at −78° C. under nitrogen atmosphere. The resulting mixture left to stir for 1.5 h at −78° C. under nitrogen atmosphere, then quenched with a saturated solution of NH₄Cl—78° C. The resulting mixture was let warm to rt then extracted with EA (3×100 mL). The combined organic layers were dried over anhydrous sodium sulfate. After filtration, the filtrate was concentrated under vacuum to afford 3-[(1s,3s)-3-((tert-butyldiphenylsilyl)oxy)cyclobutyl]-3-oxopropanenitrile (4.19 g, crude) as a yellow oil which was used in the next step without further purification. MS (ESI) calcd. for C₂₃H₂₇NO₂Si: 377.18 m/z, found: 400.15 [M+Na]⁺.

### Step 3: Synthesis of 1-(tert-butyl)-3-[(1s,3s)-3-[(tert-butyldiphenylsilyl)oxy]cyclobutyl]-1H-pyrazol-5-amine

Tert-butylhydrazine hydrochloride (1.9 g, 15.248 mmol, 1.6 equiv), NaOH (450 mg, 11.251 mmol, 1.2 equiv), a stir bar and EtOH (15 mL) were added to a 100 mL round-bottom flask and stirred for 0.5 h. Then 3-[(1s,3s)-3-((tert-butyldiphenylsilyl)oxy)cyclobutyl]-3-oxopropanenitrile (3.69 g, 9.773 mmol, 1.0 equiv) was dissolved with EtOH (15 mL), and the above mixture was added. The resulting mixture was stirred for 1.5 h at 50° C., diluted with water and extracted with EA (3×150 mL). The combined organic layer was dried over anhydrous sodium sulfate. After filtration, the filtrate was concentrated under vacuum. The residue was purified by reverse-phase chromatography (0-100% ACN/10 mM NH₄HCO₃ water) to afford 1-(tert-butyl)-3-[(1s,3s)-3-[(tert-butyldiphenylsilyl)oxy]cyclobutyl]-1H-pyrazol-5-amine (4.05 g, 92.5%) as a light yellow oil. MS (ESI) calcd. for C₂₇H₃₇N₃OSi: 447.27 m/z, found: 448.80 [M+H]⁺.

### Step 4: Synthesis of Benzyl (1-(tert-butyl)-3-((1s,3s)-3-((tert-butyldiphenylsilyl)oxy)cyclobutyl)-1H-pyrazol-5-yl)carbamate

1-(tert-butyl)-3-[(1s,3s)-3-[(tert-butyldiphenylsilyl)oxy]cyclobutyl]-1H-pyrazol-5-amine (2.0 g, 4.467 mmol, 1.0 equiv), NaHCO₃ (3.5 g, 41.664 mmol, 9.3 equiv), a stir bar and ACN (20 mL) were added to a 100 mL bottom-round flask and stirred until homogenous, then treated with a solution of benzyl chloroformate (4.7 g, 27.551 mmol, 6.2 equiv) dropwise at rt. The resulting mixture was stirred for 2 days at rt, diluted with water, and extracted with EA (3×150 mL). The combined organic layer was dried over anhydrous sodium sulfate. After filtration, the filtrate was concentrated under vacuum. The residue was purified by silica gel column chromatography, eluted with (0-25% PE/EA) to afford benzyl (1-(tert-butyl)-3-((1s,3s)-3-((tert-butyldiphenylsilyl)oxy)cyclobutyl)-1H-pyrazol-5-yl)carbamate (2.49 g, 95.8%) as a yellow oil. MS (ESI) calcd. for C₃₅H₄₃N₃O₃Si: 581.31 m/z, found: 582.45 [M+H]⁺.

### Step 5: Synthesis of Benzyl (1-(tert-butyl)-3-((1s,3s)-3-hydroxycyclobutyl)-1H-pyrazol-5-yl)carbamate

Benzyl (1-(tert-butyl)-3-((1s,3s)-3-((tert-butyldiphenylsilyl)oxy)cyclobutyl)-1H-pyrazol-5-yl)carbamate (2.4 g, 4.125 mmol, 1.0 equiv), TBAF (2.6 g, 9.962 mmol, 2.4 equiv), a stir bar and THF (24 mL) were added to a 100 mL bottom-round flask and stirred until homogenous at rt. The resulting mixture was stirred for 4 h at rt, then diluted with water and extracted with EA (3×150 mL). The combined organic layers were washed with water (5×) and brine, and dried over anhydrous sodium sulfate. After filtration, the filtrate was concentrated under vacuum. The residue was purified by reverse-phase chromatography (0-70% ACN/10 mM NH₄HCO₃ water) to afford benzyl (1-(tert-butyl)-3-((1s,3s)-3-hydroxycyclobutyl)-1H-pyrazol-5-yl)carbamate (720 mg, 50.83%) as alight yellow oil. MS (ESI) calcd. for C₁₉H₂₅N₃O₃: 343.19 m/z, found: 344.10 [M+H]⁺.

### Step 6: Synthesis of Benzyl (1-(tert-butyl)-3-((1s,3s)-3-((isopropylcarbamoyl)oxy)cyclobutyl)-1H-pyrazol-5-yl)carbamate

Benzyl (1-(tert-butyl)-3-((1s,3s)-3-hydroxycyclobutyl)-1H-pyrazol-5-yl)carbamate (700 mg, 2.038 mmol, 1.0 equiv), a stir bar, DIEA (1.33 g, 10.290 mmol, 5.1 equiv), 2-isocyanatopropane (712 mg, 8.366 mmol, 4.1 equiv), and toluene (16 mL) were added to a 40 mL vial and stirred until homogenous. The resulting mixture was stirred for 1 day at 80° C., the crude reaction was cooled to rt, diluted with water, and extracted with EA (3×150 mL). The combined organic layers were washed with brine and dried over anhydrous sodium sulfate. After filtration, the filtrate was concentrated under vacuum to afford benzyl (1-(tert-butyl)-3-((1s,3s)-3-((isopropylcarbamoyl)oxy)cyclobutyl)-1H-pyrazol-5-yl)carbamate (880 mg, crude) as a light yellow oil which was used in the next step without further purification. MS (ESI) calcd. for C₂₃H₃₂N₄O₄: 428.24 m/z, found: 429.15 [M+H]⁺.

### Step 7: Synthesis of (1s,3s)-3-(5-amino-1-tert-butylpyrazol-3-yl)cyclobutyl N-isopropylcarbamate

Benzyl (1-(tert-butyl)-3-((1s,3s)-3-((isopropylcarbamoyl)oxy)cyclobutyl)-1H-pyrazol-5-yl)carbamate (860 mg, 2.007 mmol, 1.0 equiv), a stir bar, Pd(OH)₂/C (267 mg, 1.901 mmol, 0.95 equiv) and EtOH (21 mL) were added to a 100 mL bottom-round flask and stirred until homogenous. The resulting mixture was maintained under H₂ and stirred 2 h at rt The resulting mixture was filtered and concentrated under vacuum. The residue was purified by reverse-phase chromatography (0-50% ACN/10 mM NH₄HCO₃ water) to afford (1s,3s)-3-(5-amino-1-(tert-butyl)-1H-pyrazol-3-yl)cyclobutyl isopropylcarbamate (530 mg, 89.7%) as a light yellow oil. MS (ESI) calcd. for C₁₅H₂₆N₄O₂, 294.21 m/z, found: 295.30 [M+H]⁺.

### Step 8: Synthesis of (1s,3s)-3-(5-(2-bromoacetamido)-1-(tert-butyl)-1H-pyrazol-3-yl)cyclobutyl isopropylcarbamate

(1s,3s)-3-(5-amino-1-(tert-butyl)-1H-pyrazol-3-yl)cyclobutyl isopropylcarbamate (510 mg, 1.732 mmol, 1.0 equiv), Na₂CO₃ (355 mg, 3.349 mmol, 1.9 equiv), a stir bar and THF (10 mL) were added to a 40 mL vial and stirred until homogenous, then treated with a solution of 2-bromoacetyl bromide (500 mg, 2.477 mmol, 1.4 equiv) in THF (2 mL) dropwise at rt The resulting mixture was stirred for 3 h at rt, quenched with water, and extracted with DCM (3×30 mL). The combined organic layers were washed with brine, dried over anhydrous sodium sulfate. After filtration, the filtrate was concentrated under vacuum to afford (1s,3s)-3-(5-(2-bromoacetamido)-1-(tert-butyl)-1H-pyrazol-3-yl)cyclobutyl isopropylcarbamate (820 mg, crude) as a light reddish brown oil which was used in the next step without further purification. MS (ESI) calcd. for C₁₇H₂₇BrN₄O₃: 414.13 m/z, found: 415.05 [M+H]⁺.

### Step 9: Synthesis of (1s,3s)-3-(5-(2-(3-(benzyloxy)-2-formyl-5-methoxyphenoxy)acetamido)-1-(tert-butyl)-1H-pyrazol-3-yl)cyclobutyl isopropylcarbamate

2-(benzyloxy)-6-hydroxy-4-methoxybenzaldehyde (206 mg, 0.798 mmol, 1.0 equiv), Cs₂CO₃ (535 mg, 1.642 mmol, 2.1 equiv), a stir bar and DMF (10 mL) were added to a 40 mL vial and stirred until homogenous, then treated with (1s,3s)-3-(5-(2-bromoacetamido)-1-(tert-butyl)-1H-pyrazol-3-yl)cyclobutyl isopropylcarbamate (394.21 mg, 0.950 mmol, 1.2 equiv) in batches at rt. The resulting mixture was stirred for 1.5 h at rt, diluted with water and extracted with EA (3×150 mL). The combined organic layers were washed with water (5×), brine, and dried over anhydrous sodium sulfate. After filtration, the filtrate was concentrated under vacuum. The residue was purified by silica gel column chromatography, eluted with (0-69% PE/EA) to afford (1s,3s)-3-(5-(2-(3-(benzyloxy)-2-formyl-5-methoxyphenoxy)acetamido)-1-(tert-butyl)-1H-pyrazol-3-yl)cyclobutyl isopropylcarbamate (140 mg, 29.6%) as a light yellow solid. MS (ESI) calcd. for C₃₂H₄₀N₄O₇: 592.29 m/z, found: 593.20 [M+H]⁺.

### Step 10: Synthesis of (1s,3s)-3-(3-(2-(2-formyl-3-hydroxy-5-methoxyphenoxy)acetamido)-1H-pyrazol-5-yl)cyclobutyl isopropylcarbamate

(1s,3s)-3-(5-(2-(3-(benzyloxy)-2-formyl-5-methoxyphenoxy)acetamido)-1-(tert-butyl)-1H-pyrazol-3-yl)cyclobutyl isopropylcarbamate (135 mg, 0.228 mmol, 1.0 equiv), a stir bar and formic acid (8 mL) were added to a 20 mL vial. The resulting mixture was stirred for 2.5 h at 80° C. and concentrated under vacuum. The residue was purified by reverse-phase chromatography (0-46% ACN/0.05% TFA water) to afford (1s,3s)-3-(3-(2-(2-formyl-3-hydroxy-5-methoxyphenoxy)acetamido)-1H-pyrazol-5-yl)cyclobutyl isopropylcarbamate (31.8 mg, 30.6%) as an off-white solid. MS (ESI) calcd. for C₂₁H₂₆N₄O₇: 446.18 m/z, found: 447.10 [M+H]⁺. 1H NMR (500 MHz, DMSO-d₆) δ 12.32 (s, 2H), 10.56 (s, 1H), 10.16 (s, 1H), 7.06 (d, J=7.8 Hz, 1H), 6.35 (s, 1H), 6.15 (d, J=2.1 Hz, 1H), 6.08 (d, J=2.1 Hz, 1H), 4.88-4.74 (m, 3H), 3.81 (s, 3H), 3.62-3.52 (m, 1H), 3.12-3.01 (m, 1H), 2.71-2.62 (m, 2H), 2.10-2.01 (m, 2H), 1.04 (d, J=6.5 Hz, 6H).

### Example 3.7: Synthesis of Compound 114: (1s,3s)-3-(3-(2-(2-formyl-3-hydroxyphenoxy)acetamido)-1H-pyrazol-5-yl)cyclobutyl isopropylcarbamate

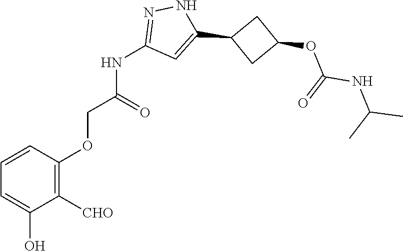 [SMILES: CC(C)NC(=O)O[C@H]1C[C@@H](c2cc(NC(=O)COc3cccc(O)c3C=O)n[nH]2)C1]

### Synthetic Route

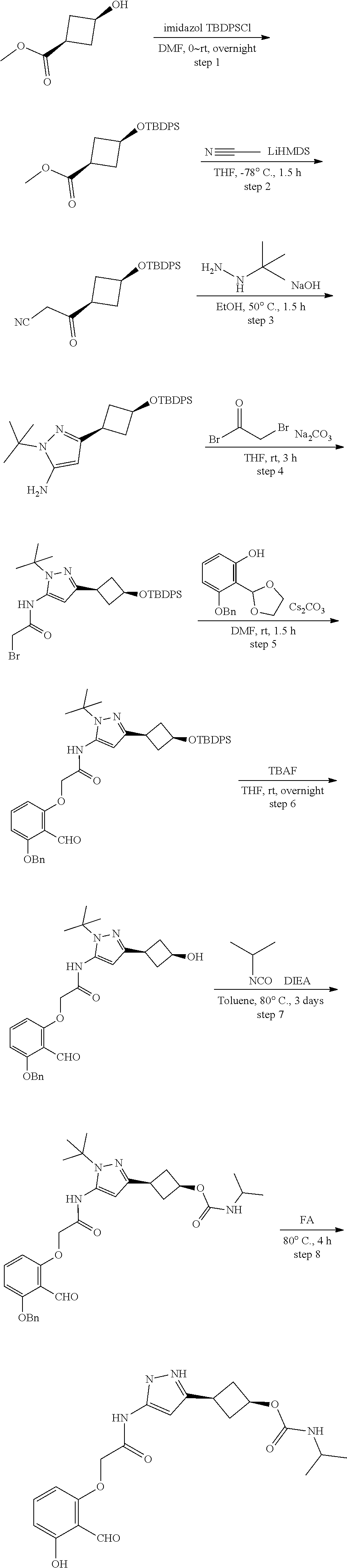

### Step 1: Synthesis of Methyl (1s,3s)-3-[(tert-butyldiphenylsilyl)oxy]cyclobutane-1-carboxylate

Methyl (1s,3s)-3-hydroxycyclobutane-1-carboxylate (1.0 g, 7.684 mmol, 1.0 equiv), a stir bar, imidazole (1.6 g, 23.502 mmol, 3.1 equiv) and DMF (10 mL) were added to a 40 mL vial and stirred until homogenous at 0° C. The reaction mixture was then treated with tert-butylchlorodiphenylsilane (5.3 g, 19.283 mmol, 2.5 equiv) dropwise at 0° C. The resulting mixture was stirred for overnight at rt. The resulting mixture was diluted with water and extracted with EA (3×100 mL). The combined organic layers were washed water (5×) and brine, and dried over anhydrous sodium sulfate. After filtration, the filtrate was concentrated under vacuum. The residue was purified by silica gel column chromatography, eluted with (0-11% PE/EA) to afford methyl (1s,3s)-3-[(tert-butyldiphenylsilyl)oxy]cyclobutane-1-carboxylate (2.48 g, 94.6%) as a colorless oil. MS (ESI) calcd. for C₂₂H₂₈O₃Si: 368.18 m/z, found: 391.20 [M+Na]⁺.

### Step 2: Synthesis of 3-((1s,3s)-3-[(tert-butyldiphenylsilyl)oxy)cyclobutyl]-3-oxopropanenitrile

Methyl (1s,3s)-3-[(tert-butyldiphenylsilyl)oxy]cyclobutane-1-carboxylate (1.1 g, 2.985 mmol, 1.0 equiv), a stir bar, THF (10 mL) and acetonitrile (371.0 mg, 9.0 mmol, 3.0 equiv) were added to a 50 mL round-bottom flask and stirred until homogenous at −78° C. under nitrogen atmosphere. The reaction mixture was then treated with a solution of LiHMDS (6.0 mL, 6.000 mmol, 2.0 equiv, 1 mol/L) dropwise at −78° C. under nitrogen atmosphere. The resulting mixture was stirred for 1.5 h at −78° C. under nitrogen atmosphere, and quenched with a saturated solution of NH₄Cl. The resulting mixture was let warm to rt then extracted with EA (3×50 mL). The combined organic layer was dried over anhydrous sodium sulfate. After filtration, the filtrate was concentrated under vacuum to afford 3-[(1s,3s)-3-((tert-butyldiphenylsilyl)oxy)cyclobutyl]-3-oxopropanenitrile (1.16 g, crude) as a light yellow oil which was used in the next step without further purification. MS (ESI) calcd. for C₂₃H₂₇NO₂Si: 377.18 m/z, found: 400.205 [M+Na]⁺.

### Step 3: Synthesis of 1-(tert-butyl)-3-[(1s,3s)-3-[(tert-butyldiphenylsilyl)oxy]cyclobutyl]-1H-pyrazol-5-amine

tert-Butylhydrazine hydrochloride (246.0 mg, 1.974 mmol, 1.5 equiv), NaOH (54 mg, 1.350 mmol, 1.0 equiv), a stir bar and EtOH (2.5 mL) were added to a 20 mL vial and stirred for 0.5 h. Then 3-[(1s,3s)-3-((tert-butyldiphenylsilyl)oxy)cyclobutyl]-3-oxopropanenitrile (500 mg, 1.324 mmol, 1 equiv) was dissolved with EtOH (2.5 mL), and the above mixture was added. The resulting mixture was stirred for 1.5 h at 50° C., diluted with water and extracted with EA (3×50 mL). The combined organic layer was dried over anhydrous sodium sulfate. After filtration, the filtrate was concentrated under vacuum. The residue was purified by reverse-phase chromatography (0-80% ACN/10 mM NH₄HCO₃ water) to afford 1-(tert-butyl)-3-[(1s,3s)-3-[(tert-butyldiphenylsilyl)oxy]cyclobutyl]-1H-pyrazol-5-amine (590 mg, 97.5%) as a colorless oil. MS (ESI) calcd. for C₂₇H₃₇N₃OSi: 447.27 m/z, found: 448.80 [M+H]⁺.

### Step 4: Synthesis of 2-bromo-N-(1-(tert-butyl)-3-((1s,3s)-3-((tert-butyldiphenylsilyl)oxy)cyclobutyl)-1H-pyrazol-5-yl)acetamide

1-(tert-butyl)-3-[(1s,3s)-3-[(tert-butyldiphenylsilyl)oxy]cyclobutyl]-1H-pyrazol-5-amine (220 mg, 0.491 mmol, 1.0 equiv), Na₂CO₃ (82 mg, 0.766 mmol, 1.6 equiv), a stir bar and THF (3 mL) were added to a 25 mL round-bottom flask and stirred until homogenous, then treated with a solution of 2-bromoacetyl bromide (155 mg, 0.768 mmol, 1.6 equiv) in THF (0.5 mL) dropwise at rt. The resulting mixture was stirred for 3 h at rt. The resulting mixture was diluted with water and extracted with DCM (3×30 mL). The combined organic layers were washed with brine, and dried over anhydrous sodium sulfate. After filtration, the filtrate was concentrated under vacuum to afford 2-bromo-N-(1-(tert-butyl)-3-((1s,3s)-3-((tert-butyldiphenylsilyl)oxy)cyclobutyl)-1H-pyrazol-5-yl)acetamide (335 mg, crude) as a light yellow oil which was used in the next step without further purification. MS (ESI) calcd. for C₂₉H₃₈BrN₃O₂Si: 567.19 m/z, found: 568.20 [M+H]⁺.

### Step 5: Synthesis of 2-(3-(benzyloxy)-2-formylphenoxy)-N-(1-(tert-butyl)-3-((1s,3s)-3-((tert-butyldiphenylsilyl)oxy)cyclobutyl)-1H-pyrazol-5-yl)acetamide

3-(benzyloxy)-2-(1,3-dioxolan-2-yl)phenol (91 mg, 0.334 mmol, 1.0 equiv), Cs₂CO₃ (229 mg, 0.701 mmol, 2.1 equiv), a stir bar and DMF (2 mL) were added to a 25 mL round-bottom flask and stirred until homogenous, then treated with a solution of 2-bromo-N-(1-(tert-butyl)-3-((1s,3s)-3-((tert-butyldiphenylsilyl)oxy)cyclobutyl)-1H-pyrazol-5-yl)acetamide (233 mg, 0.410 mmol, 1.2 equiv) in DMF (0.3 mL) dropwise at rt. The resulting mixture was stirred for 1.5 h at rt. The resulting mixture was diluted with water and extracted with EA (3×20 mL). The combined organic layers were washed with water (5×), brine, and dried over anhydrous sodium sulfate. After filtration, the filtrate was concentrated under vacuum. The residue was purified by silica gel column chromatography, eluted with (0-100% PE/EA) to afford 2-(3-(benzyloxy)-2-formylphenoxy)-N-(1-(tert-butyl)-3-((1s,3s)-3-((tert-butyldiphenylsilyl)oxy)cyclobutyl)-1H-pyrazol-5-yl)acetamide (139 mg, 54.7%) as a light pink oil. MS (ESI) calcd. for C₄₅H₅₃N₃O₆Si: 759.37 m/z, found: 760.55 [M+H]⁺.

### Step 6: Synthesis of 2-(3-(benzyloxy)-2-formylphenoxy)-N-(1-(tert-butyl)-3-((1s,3s)-3-hydroxycyclobutyl)-1H-pyrazol-5-yl)acetamide

2-(3-(benzyloxy)-2-formylphenoxy)-N-(1-(tert-butyl)-3-((1s,3s)-3-((tert-butyldiphenylsilyl)oxy)cyclobutyl)-1H-pyrazol-5-yl)acetamide (120 mg, 0.158 mmol, 1.0 equiv), a stir bar, and THF (8 mL) were added to a 25 mL round-bottom flask and stirred until homogenous at rt. The reaction mixture was then treated with TBAF (122 mg, 0.386 mmol, 2.5 equiv) in batches. The resulting mixture was stirred for overnight at rt. The reaction mixture concentrated under vacuum, diluted with water, and extracted with DCM (3×20 mL). The combined organic layers were washed with water (5×), brine, and dried over anhydrous sodium sulfate. After filtration, the filtrate was concentrated under vacuum. The residue was purified by silica gel column chromatography, eluted with (0-5% DCM/MeOH) to afford 2-(3-(benzyloxy)-2-formylphenoxy)-N-(1-(tert-butyl)-3-((1s,3s)-3-hydroxycyclobutyl)-1H-pyrazol-5-yl)acetamide (48 mg, 58.3%) as a light yellow oil. MS (ESI) calcd. for C₂₉H₃₅N₃O₆: 521.25 m/z, found: 522.15 [M+H]⁺.

### Step 7: Synthesis of (1s,3s)-3-(5-(2-(3-(benzyloxy)-2-formylphenoxy)acetamido)-1-(tert-butyl)-1H-pyrazol-3-yl)cyclobutyl isopropylcarbamate

2-(3-(benzyloxy)-2-formylphenoxy)-N-(1-(tert-butyl)-3-((1s,3s)-3-hydroxycyclobutyl)-1H-pyrazol-5-yl)acetamide (43 mg, 0.082 mmol, 1.0 equiv), a stir bar, DIEA (36 mg, 0.279 mmol, 3.4 equiv), 2-isocyanatopropane (15 mg, 0.176 mmol, 2.1 equiv), and toluene (4 mL) were added to a 25 mL round-bottom flask and stirred until homogenous under N₂. The resulting mixture was stirred for 3 days at 80° C. and concentrated under vacuum. The residue was purified by silica gel column chromatography, eluted with (0-68% PE/EA) to afford (1s,3s)-3-(5-(2-(3-(benzyloxy)-2-formylphenoxy)acetamido)-1-(tert-butyl)-1H-pyrazol-3-yl)cyclobutyl isopropylcarbamate (42 mg, 84.0%) as a light yellow solid. MS (ESI) calcd. for C₃₃H₄₂N₄O₇: 606.31 m/z, found: 607.20 [M+H]⁺.

### Step 8: Synthesis of (1s,3s)-3-(3-(2-(2-formyl-3-hydroxyphenoxy)acetamido)-1H-pyrazol-5-yl)cyclobutyl isopropylcarbamate

(1s,3s)-3-(5-(2-(3-(benzyloxy)-2-formylphenoxy)acetamido)-1-(tert-butyl)-1H-pyrazol-3-yl)cyclobutyl isopropylcarbamate (37 mg, 0.061 mmol, 1.0 equiv), a stir bar and FA (2.5 mL) were added to a 8 mL vial. The resulting mixture was stirred for 4 h at 80° C. and concentrated under vacuum. The residue was purified by Prep-HPLC with (Column: XSelect CSH Fluoro Phenyl 30*150 mm, 5 μm; Mobile Phase A: Water (0.1% FA), Mobile Phase B: ACN; Flow rate: 60 mL/min; Gradient: 43% B to 73% B in 10 min; Wave Length: 254/220 nm; RT1 (min): 6.03) to afford (1s,3s)-3-(3-(2-(2-formyl-3-hydroxyphenoxy)acetamido)-1H-pyrazol-5-yl)cyclobutyl isopropylcarbamate (4.6 mg, 18.08%) as a white solid. MS (ESI) calcd. for C₂₀H₂₄N₄O₆: 416.17 m/z, found: 417.15 [M+H]⁺. ¹H NMR (500 MHz, DMSO-d₆) δ 11.71 (s, 1H), 10.55 (d, J=2.0 Hz, 1H), 10.40 (s, 1H), 7.51 (t, J=8.4 Hz, 1H), 7.06 (d, J=7.9 Hz, 1H), 6.55 (dd, J=21.3, 8.4 Hz, 2H), 6.35 (s, 1H), 4.82 (d, J=11.5 Hz, 3H), 3.62-3.50 (m, 1H), 3.12-3.01 (m, 1H), 2.71-2.62 (m, 2H), 2.10-2.01 (m, 2H), 1.04 (d, J=6.6 Hz, 6H).

### Example 3.8: Synthesis of Compound 111: (1s,3s)-3-(3-(2-(2-formyl-3-hydroxy-5-methoxyphenoxy)acetamido)-1H-pyrazol-5-yl)cyclobutyl 3-oxa-6-azabicyclo[3.1.1]heptane-6-carboxylate

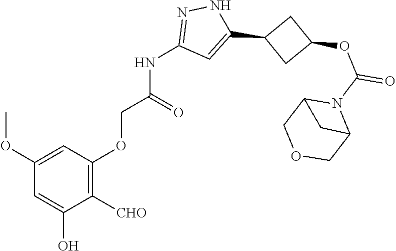 [SMILES: COc1cc(O)c(C=O)c(OCC(=O)Nc2cc([C@H]3C[C@@H](OC(=O)N4C5COCC4C5)C3)[nH]n2)c1]

### Synthetic Route

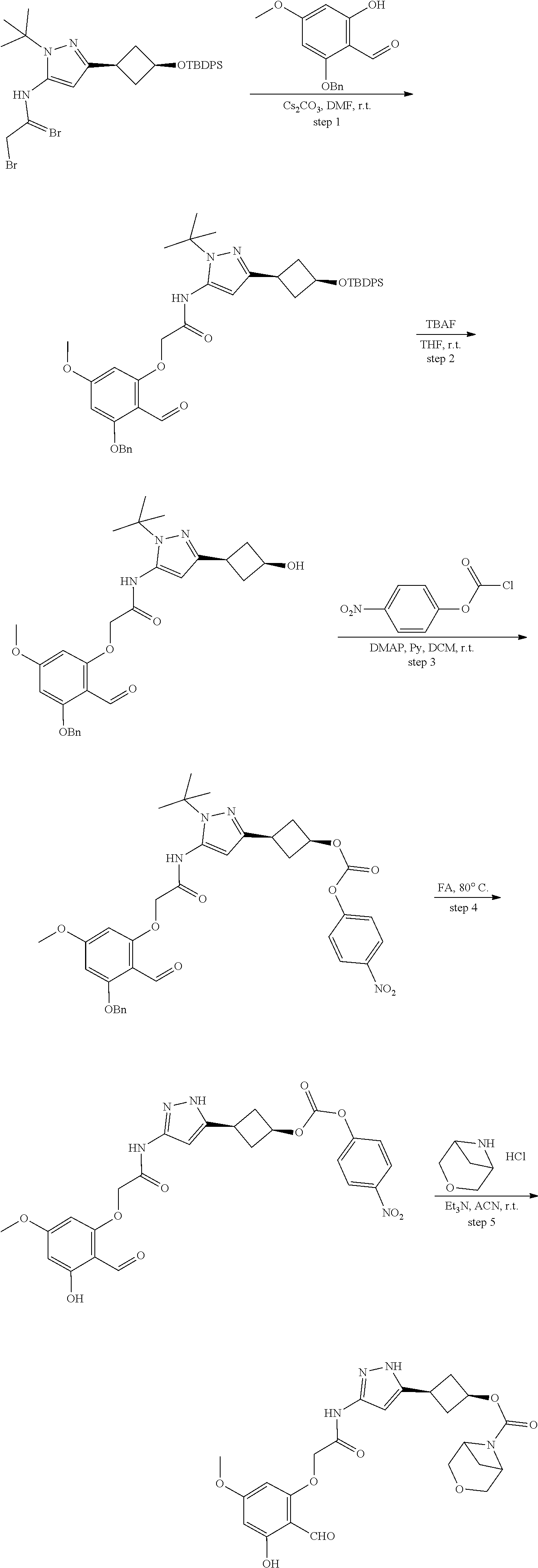

### Step 1: Synthesis of 2-(3-(benzyloxy)-2-formyl-5-methoxyphenoxy)-N-(1-(tert-butyl)-3-((1s,3s)-3-((tert-butyldiphenylsilyl)oxy)cyclobutyl)-1H-pyrazol-5-yl)acetamide

2-bromo-N-(2-tert-butyl-5-[(1s,3s)-3-[(tert-butyldiphenylsilyl)oxy]cyclobutyl]pyrazol-3-ylacetamide (6.60 g, 11.616 mmol, 1.5 equiv), a stir bar, Cs₂CO₃ (6.31 g, 19.360 mmol, 2.50 equiv) and DMF (50 mL) were added to a 100 mL round bottom flask and stirred until homogenous, then treated with 2-(benzyloxy)-6-hydroxy-4-methoxybenzaldehyde (2 g, 7.744 mmol, 1.00 equiv) in DMF (5 mL) dropwise at room temperature. The resulting mixture was stirred for 1 h at room temperature, diluted with water (50 mL) and extracted with DCM (100 mL×3). The combined organic layers were washed with water (100 mL×5), brine, dried over anhydrous sodium sulfate, filtered, and concentrated under vacuum pressure. The residue was purified by silica gel chromatography (0-52% PE/EA) to give 2-(3-(benzyloxy)-2-formyl-5-methoxyphenoxy)-N-(1-(tert-butyl)-3-((1s,3s)-3-((tert-butyldiphenylsilyl)oxy)cyclobutyl)-1H-pyrazol-5-yl)acetamide (2.3 g, 39.81%) as a yellow solid. MS (ESI) mass cacld. for C₄₄H₅₁N₃O₆Si, 745.36 m/z, found, 746.45 [M+H]⁺.

### Step 2: Synthesis of 2-(3-(benzyloxy)-2-formyl-5-methoxyphenoxy)-N-(1-(tert-butyl)-3-((1s,3s)-3-hydroxycyclobutyl)-1H-pyrazol-5-yl)acetamide

2-(3-(benzyloxy)-2-formyl-5-methoxyphenoxy)-N-(1-(tert-butyl)-3-((1s,3s)-3-((tert-butyldiphenylsilyl)oxy)cyclobutyl)-1H-pyrazol-5-yl)acetamide (2.7 g, 3.619 mmol, 1 equiv), a stir bar, and THF (38 mL) were added to a 100 mL round bottom flask and stirred until homogenous. The resulting solution was then treated with a solution of TBAF (1.135 g, 4.341 mmol, 1.20 equiv) in THF (2 mL) dropwise at room temperature. The resulting mixture was stirred overnight at room temperature, followed by dilution with water (50 mL), and extracted with DCM (100 mL×3). The combined organic layers were washed with brine, dried over anhydrous sodium sulfate, filtered, and concentrated under vacuum pressure. The residue was purified by silica gel chromatography (0-100% PE/EA) to give 2-(3-(benzyloxy)-2-formyl-5-methoxyphenoxy)-N-(1-(tert-butyl)-3-((1s,3s)-3-hydroxycyclobutyl)-1H-pyrazol-5-yl)acetamide (920 mg, 50.08%) as a white solid. MS (ESI) mass cacld. for C₂₈H₃₃N₃O₆, 507.24 m/z, found, 508.30 [M+H]⁺.

### Step 3: Synthesis of (1s,3s)-3-(5-(2-(3-(benzyloxy)-2-formyl-5-methoxyphenoxy)acetamido)-1-(tert-butyl)-1H-pyrazol-3-yl)cyclobutyl (4-nitrophenyl) carbonate

2-(3-(benzyloxy)-2-formyl-5-methoxyphenoxy)-N-(1-(tert-butyl)-3-((1s,3s)-3-hydroxycyclobutyl)-1H-pyrazol-5-yl)acetamide (900 mg, 1.773 mmol, 1 equiv), a stir bar, DCM (15 mL), 4-nitrophenyl carbonochloridate (536 mg, 2.659 mmol, 1.50 equiv) and pyridine (421 mg, 5.322 mmol, 3.00 equiv) were added to a 50 mL round bottom flask and stirred until homogeneous, then treated with DMAP (222 mg, 1.817 mmol, 1.02 equiv) in batches at room temperature. The resulting mixture was stirred for 1 h at room temperature, diluted with water (15 mL) and extracted with DCM (30 mL×3). The combined organic layers were washed with brine, dried with anhydrous sodium sulfate, filtered, and concentrated under vacuum pressure. The residue was purified by silica gel chromatography (0-63% PE/EA) to give (1s,3s)-3-(5-(2-(3-(benzyloxy)-2-formyl-5-methoxyphenoxy)acetamido)-1-(tert-butyl)-1H-pyrazol-3-yl)cyclobutyl (4-nitrophenyl) carbonate (1 g, 83.84%) as a white solid. MS (ESI) mass cacld. for C₃₅H₃₆N₄O₁₀, 672.24 m/z, found, 673.20 [M+H]⁺.

### Step 4: Synthesis of (1s,3s)-3-(3-(2-(2-formyl-3-hydroxy-5-methoxyphenoxy)acetamido)-1H-pyrazol-5-yl)cyclobutyl (4-nitrophenyl) carbonate

(1s,3s)-3-(5-(2-(3-(benzyloxy)-2-formyl-5-methoxyphenoxy)acetamido)-1-(tert-butyl)-1H-pyrazol-3-yl)cyclobutyl (4-nitrophenyl) carbonate (1 g, 1.487 mmol, 1 equiv), a stir bar and FA (15 mL) were added to a 40 mL vial and stirred until homogeneous. The resulting mixture was stirred for 5 h at 80° C., cooled to room temperature, concentrated under vacuum pressure to give (1s,3s)-3-(3-(2-(2-formyl-3-hydroxy-5-methoxyphenoxy)acetamido)-1H-pyrazol-5-yl)cyclobutyl (4-nitrophenyl) carbonate (1.042 g, crude) as a reddish oil which was used in the next step without further purification. MS (ESI) mass cacld. for C₂₄H₂₂N₄O₁₀, 526.13 m/z, found, 527.20 [M+H]⁺.

### Step 5: Synthesis of (1s,3s)-3-(3-(2-(2-formyl-3-hydroxy-5-methoxyphenoxy)acetamido)-1H-pyrazol-5-yl)cyclobutyl 3-oxa-6-azabicyclo[3.1.1]heptane-6-carboxylate

(1s,3s)-3-(3-(2-(2-formyl-3-hydroxy-5-methoxyphenoxy)acetamido)-1H-pyrazol-5-yl)cyclobutyl (4-nitrophenyl) carbonate (130 mg, 0.247 mmol, 1 equiv), a stir bar, Et₃N (58 mg, 0.573 mmol, 2.32 equiv) and ACN (4 mL) were added to a 20 mL vial and stirred until homogeneous, then treated with a solution of 3-oxa-6-azabicyclo[3.1.1]heptane (20.5 mg, 0.207 mmol, 0.84 equiv) in ACN (0.5 mL) at 0° C. The resulting mixture was stirred for 2 h at room temperature, diluted with water (10 mL) and extracted with DCM (30 mL×3). The combined organic layers were washed with brine, dried over anhydrous sodium sulfate, filtered, and concentrated under vacuum pressure. The residue was purified by reverse phase chromatography (0-58% ACN/H₂O) to give (1s,3s)-3-(3-(2-(2-formyl-3-hydroxy-5-methoxyphenoxy)acetamido)-1H-pyrazol-5-yl)cyclobutyl 3-oxa-6-azabicyclo[3.1.1]heptane-6-carboxylate (13.1 mg, 10.13%) as a light yellow solid. MS (ESI) mass cacld. for C₂₃H₂₆N₄O₈, 486.18 m/z, found, 487.25 [M+H]⁺; 1H NMR (400 MHz, DMSO-d₆) δ 12.32 (s, 1H), 12.26 (s, 1H), 10.56 (s, 1H), 10.15 (s, 1H), 6.36 (s, 1H), 6.14 (d, J=2.1 Hz, 1H), 6.07 (d, J=2.2 Hz, 1H), 4.94-4.83 (m, 1H), 4.81 (s, 2H), 4.12-3.99 (m, 4H), 3.81 (s, 3H), 3.70 (d, J=10.5 Hz, 2H), 3.14-3.02 (m, 1H), 2.72-2.64 (m, 2H), 2.57-2.51 (m, 1H), 2.16-2.04 (m, 2H), 1.63 (d, J=8.3 Hz, 1H).

### Example 4 Representative Schemes following General Scheme C

### Example 4.1: Synthesis of Compound 94: (1R,3S)-3-(3-((1s,3R)-3-(2-formyl-3-hydroxyphenoxy)cyclobutane-1-carboxamido)-1H-pyrazol-5-yl)cyclopentyl and compound 1; and (1R,3S)-3-(3-((1r,3S)-3-(2-formyl-3-hydroxyphenoxy) cyclobutane-1-carboxamido)-1H-pyrazol-5-yl)cyclopentyl isopropylcarbamate isopropylcarbamate

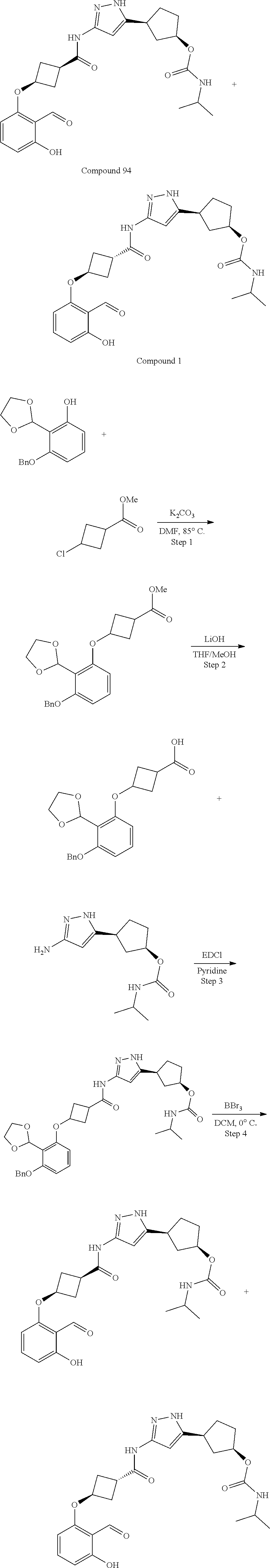

### Step 1: Synthesis of methyl 3-(3-(benzyloxy)-2-(1,3-dioxolan-2-yl)phenoxy)cyclobutane-1-carboxylate

3-(benzyloxy)-2-(1,3-dioxolan-2-yl)phenol (300.0 mg, 1.102 mmol), Cs₂CO₃ (718.0 mg, 2.204 mmol) and DMF (10 mL) were added into 40 mL vial. The reaction mixture was stirred at 20° C. for 20 min under nitrogen-purged. Then methyl 3-chlorocyclobutane-1-carboxylate (246.0 mg, 1.653 mmol) was added into this mixture. The result mixture was stirred at 85° C. for 12 h. After cooling down to rt, the mixture was diluted with EtOAc (100 mL). The organic layer was washed with H₂O (20 mL×5) and dried over Na₂SO₄, filtered and concentrated under vacuum to afford the residue. The residue was purified by silica gel chromatography (0-60% EtOAc/petroleum ether) to afford methyl 3-(3-(benzyloxy)-2-(1,3-dioxolan-2-yl)phenoxy)cyclobutane-1-carboxylate (380.0 mg, 89.72%) as a yellow oil, MS (ESI) calcd. for C₂₂H₂₄O₆, 384.16 m/z, found: 385.15 [M+H]⁺.

### Step 2: Synthesis of 3-(3-(benzyloxy)-2-(1,3-dioxolan-2-yl)phenoxy)cyclobutane-1-carboxylic acid

Methyl 3-(3-(benzyloxy)-2-(1,3-dioxolan-2-yl)phenoxy)cyclobutane-1-carboxylate (350.0 mg, 0.910 mmol), LiOH (0.91 mL, 2.730 mmol), MeOH (6 mL), a stir bar and THF (6 mL) were added to an oven-dried 8 mL vial and stirred until homogenous. The resulting mixture was stirred for 2 h at rt. The reaction mixture was monitored by LCMS. The resulting mixture was diluted with H₂O (15 mL). Then the mixture was adjusted to pH=6 with 1 M HCl solution. The resulting mixture was extracted with MeOH/DCM=1/10, v/v (50 mL×3). The combined organic layers were dried over Na₂SO₄, filtered and concentrated under vacuum. The crude product 3-(3-(benzyloxy)-2-(1,3-dioxolan-2-yl)phenoxy)cyclobutane-1-carboxylic acid (400.0 mg, crude) was used in the next step directly without further purification, MS (ESI) calcd. for C₂₁H₂₂O₆, 370.14 m/z, found: 371.15 [M+H]⁺.

### Step 3: Synthesis of (1R,3S)-3-(3-(3-(3-(benzyloxy)-2-(1,3-dioxolan-2-yl)phenoxy) cyclobutane-1-carboxamido)-1H-pyrazol-5-yl)cyclopentyl isopropylcarbamate

3-(3-(benzyloxy)-2-(1,3-dioxolan-2-yl)phenoxy)cyclobutane-1-carboxylic acid (200.0 mg, 0.540 mmol) and pyridine (10 mL) were added to an 50 mL flask and stirred until homogenous, then treated with EDCI (207.0 mg, 1.080 mmol). After 5 mins, then (1R,3S)-3-(5-amino-2H-pyrazol-3-yl)cyclopentyl N-isopropylcarbamate (150.0 mg, 0.594 mmol) was added into this mixture. The resulting mixture was stirred at rt for 2 h, then concentrated under vacuum. The residue was purified by reverse-phase chromatography ACN/H₂O (10 mM NH₄HCO₃) 5-80% to afford (1R,3S)-3-(3-(3-(3-(benzyloxy)-2-(1,3-dioxolan-2-yl)phenoxy)cyclobutane-1-carboxamido)-1H-pyrazol-5-yl)cyclopentyl isopropylcarbamate (130 mg, 39.81%) as a yellow solid, MS (ESI) calcd. for C₃₃H₄₀N₄O₇, 604.29 m/z, found: 605.25 [M+H]⁺.

### Step 4: Synthesis of (1R,3S)-3-(3-((1r,3S)-3-(2-formyl-3-hydroxyphenoxy)cyclobutane-1-carboxamido)-1H-pyrazol-5-yl)cyclopentyl isopropylcarbamate

(1R,3S)-3-(3-(3-(3-(benzyloxy)-2-(1,3-dioxolan-2-yl)phenoxy)cyclobutane-1-carboxamido)-1H-pyrazol-5-yl)cyclopentyl isopropylcarbamate (100.0 mg, 0.165 mmol), a stir bar and DCM (5 mL) were added to a 50 mL flask and stirred until homogenous, then treated with boron tribromide (1.0 mL, 1.000 mmol) at 0° C. The resulting mixture was stirred at rt for 1 h. Then the reaction was quenched with H₂O 10 mL and extracted with DCM (20 mL×3). The organic layers were combined, dried over Na₂SO₄, filtered and concentrated. The residue was obtained as yellow oil. The residue obtained was purified by Prep-HPLC (Column: Xselect CSH Prep C18 OBD Column, 30×150 mm, 5 um; Mobile Phase A: Water (0.05% TFA), Mobile Phase B: ACN; Flow rate: 60 mL/min; Gradient: 33% B to 53% B in 10 min; Wave Length: 254/220 nm; RT1 (min): 8.5; RT2 (min): 9.1) to give two products. The first eluting peak was lyophilized to afford (1R,3S)-3-(3-((1s,3R)-3-(2-formyl-3-hydroxyphenoxy)cyclobutane-1-carboxamido)-1H-pyrazol-5-yl)cyclopentyl isopropylcarbamate (6.9 mg, 8.81%) as a white solid. MS (ESI) calcd. for C₂₄H₃₀N₄O₆, 470.22 m/z, found, 471.25 m/z [M+H]⁺. ¹H NMR (400 MHz, DMSO-d₆) δ 11.75 (s, 1H), 10.37 (s, 1H), 10.29 (s, 1H), 7.50 (t, J=8.4 Hz, 1H), 6.96 (d, J=6.8 Hz, 1H), 6.52-6.47 (m, 2H), 6.33 (s, 1H), 4.99 (s, 1H), 4.81-4.79 (m, 1H), 3.62-3.49 (m, 2H), 3.08-2.99 (m, 1H), 2.97-2.88 (m, 1H), 2.72-2.61 (m, 2H), 2.49-2.44 (m, 1H), 2.34-2.24 (m, 2H), 2.03-1.95 (m, 1H), 1.95-1.81 (m, 1H), 1.75-1.63 (m, 2H), 1.58 (m, 1H), 1.03 (d, J=6.5 Hz, 6H). The second eluting was lyophilized to afford (1R,3S)-3-(3-((1r,3S)-3-(2-formyl-3-hydroxyphenoxy)cyclobutane-1-carboxamido)-1H-pyrazol-5-yl)cyclopentyl isopropylcarbamate (5.3 mg, 6.72%) as a white solid. MS (ESI) calcd. for C₂₄H₃₀N₄O₆, 470.22, found, 471.20 [M+H]⁺. ¹H NMR (400 MHz, DMSO-d₆) δ 11.75 (s, 1H), 10.34 (d, J=5.6 Hz, 2H), 7.50 (t, J=8.4 Hz, 1H), 6.97 (d, J=7.6 Hz, 1H), 6.52 (d, J=8.4 Hz, 1H), 6.40-6.33 (m, 2H), 5.02-4.93 (m, 2H), 3.68-3.55 (m, 2H), 3.30-3.29 (m, 1H), 3.09-3.00 (m, 1H), 2.68-2.60 (m, 2H), 2.49-2.41 (m, 3H), 2.04-1.96 (m, 1H), 1.94-1.85 (m, 1H), 1.69-1.65 (m, 2H), 1.59 (s, 1H), 1.04 (d, J=6.4 Hz, 6H).

### Example 4.2: Synthesis of Compound 11: (1R,3S)-3-(3-(2-(3-formyl-2-hydroxyphenoxy) acetamido)-1H-pyrazol-5-yl)cyclopentyl isopropylcarbamate

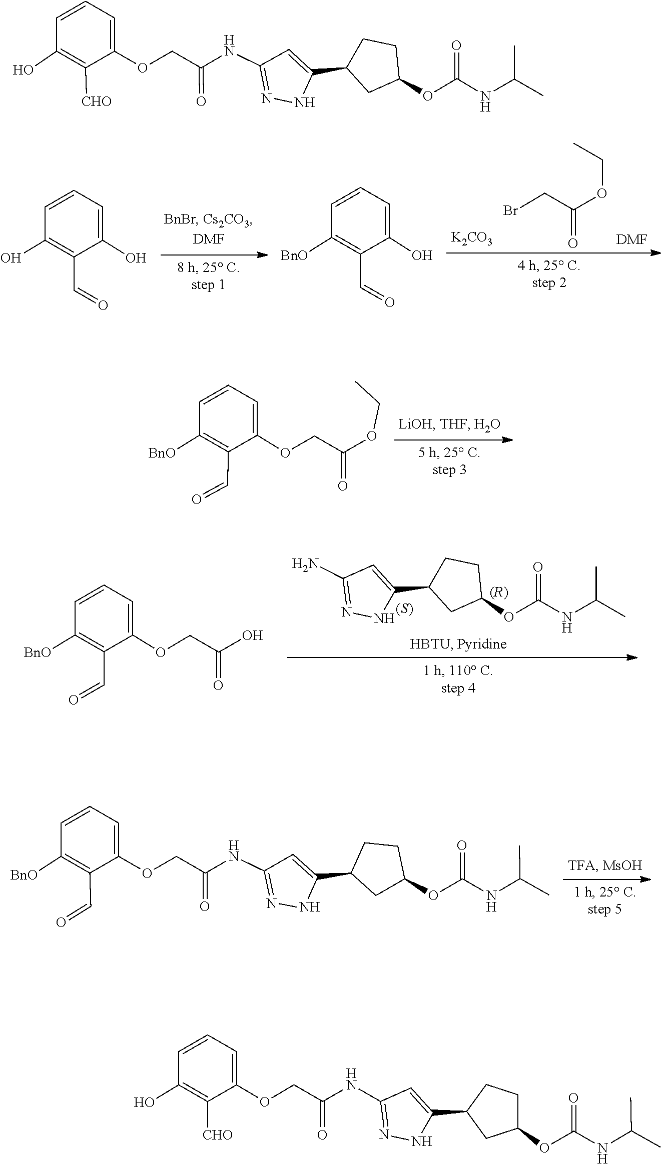

### Step 1: 2-(benzyloxy)-6-hydroxybenzaldehyde

A solution of 2,6-dihydroxybenzaldehyde (5 g, 36.200 mmol, 1 equiv) in DMF (50 mL) was treated with Cs₂CO₃ (7.08 g, 21.720 mmol, 0.6 equiv) for 30 mins at 25° C. followed by the addition of BnBr (9.29 g, 54.300 mmol, 1.5 equiv) dropwise at 25° C. The mixture was stirred for 8 h at 25° C. The resulting mixture was diluted with EA (100 mL). The combined organic layers were washed with H₂O (50 mL×2), dried over anhydrous Na₂SO₄. After filtration, the filtrate was concentrated under reduced pressure. The residue was purified by silica gel column with PE/EA (1:3) to afford 2-(benzyloxy)-6-hydroxybenzaldehyde (5.81 g, 65.04%) as a yellow solid. MS (ESI) calcd for C₁₄H₁₂O₃, 228.21 m/z, found 229.21 [M+H]⁺.

### Step 2: ethyl 2-[3-(benzyloxy)-2-formylphenoxy]acetate

A solution of 2-(benzyloxy)-6-hydroxybenzaldehyde (5.81 g, 25.455 mmol, 1 equiv) in DMF (20 mL, 129.215 mmol) was treated with K₂CO₃ (5.28 g, 38.182 mmol, 1.5 equiv) for 30 inns at 25° C. followed by the addition of ethyl bromoacetate (5.10 g, 30.546 mmol, 1.2 equiv) dropwise at 25° C. The mixture was stirred for 8 h at 25° C. The mixture was diluted with EA (20 mL). The resulting mixture was washed with H₂O (20 mL×3), dried over anhydrous Na₂SO₄. After filtration, the filtrate was concentrated under reduced pressure. After filtration, the filtrate was concentrated under reduced pressure. The residue was purified by silica gel column with EA:PE (1:1) to afford ethyl 2-[3-(benzyloxy)-2-formylphenoxy]acetate (7 g, 86.87%) as a yellow solid. MS (ESI) calcd. for C₁₈H₁₈O₅, 314.25 m/z, found 337.25 [M+Na]⁺.

### Step 3: 3-(benzyloxy)-2-formylphenoxyacetic acid

A solution of methyl 2-[3-(benzyloxy)-2-formylphenoxy]acetate (250 mg, 0.832 mmol, 1 equiv) and LiOH (27.9 mg, 1.165 mmol, 1.4 equiv) in THF (5 mL) and H₂O (1 mL) was stirred for 5 h at 25° C. The resulting mixture was concentrated under reduced pressure. The mixture was neutralized to pH 7 with HCl (2 M). The mixture was extracted with EA (10 mL). The combined organic layers were washed with H₂O (10 mL×3), dried over anhydrous Na₂SO₄. After filtration, the filtrate was concentrated under reduced pressure. This resulted in 3-(benzyloxy)-2-formylphenoxyacetic acid (125 mg, 48.41%) as a white solid. MS (ESI) calcd. for C₁₆H₁₄O₅, 286.38 m/z, found 287.38 [M+H]⁺.

### Step 4: (1R,3S)-3-(5-{2-[3-(benzyloxy)-2-formylphenoxy]acetamido}-2H-pyrazol-3-yl)cyclo-pentyl N-isopropylcarbamate

A solution of 3-(benzyloxy)-2-formylphenoxyacetic acid (150 mg, 0.524 mmol, 1 equiv) and (1R,3S)-3-(5-amino-2H-pyrazol-3-yl)cyclopentyl N-isopropylcarbamate (132.2 mg, 0.524 mmol, 1 equiv) and HBTU (298.1 mg, 0.786 mmol, 1.5 equiv) in pyridine (5 mL) was stirred for 1 h at 110° C. The mixture was diluted with EA (10 mL) and washed with H₂O (10 mL×3), dried over anhydrous Na₂SO₄. After filtration, the filtrate was concentrated under reduced pressure. This resulted in (1R,3S)-3-(5-{2-[3-(benzyloxy)-2-formylphenoxy]acetamido}-2H-pyrazol-3-yl)cyclopentyl N-isopropylcarbamate (100 mg, 30.83%) as a yellow solid. MS (ESI) calcd. for C₂₈H₃₂N₄O₆, 520.2 m/z, found 521.2 [M+H]⁺.

### Step 5: (1R,3S)-3-{5-[2-(2-formyl-3-hydroxyphenoxy)acetamido]-2H-pyrazol-3-yl}cyclo-pentyl N-isopropylcarbamate

A solution of (1R,3S)-3-(5-{2-[3-(benzyloxy)-2-formylphenoxy]acetamido}-2H-pyrazol-3-yl)cyclopentyl N-isopropylcarbamate (100 mg, 0.192 mmol, 1 equiv) in DCM (0.5 mL) and MsOH (0.1 mL) was stirred for 1 h at 25° C. Desired product could be detected by LCMS The crude product was purified by Prep-HPLC with the following conditions (Column: Xselect CSH C18 OBD Column 30×150 mm 5 μm, n; Mobile Phase A: Water (0.1% FA), Mobile Phase B: ACN; Flow rate: 60 mL/min; Gradient: 12% B to 38% B in 9 min, 38% B; Wave Length: 254/220 nm; RT1 (min): 9; to afford (1R,3S)-3-{5-[2-(2-formyl-3-hydroxyphenoxy)acetamido]-2H-pyrazol-3-yl}cyclopentyl N-isopropylcarbamate (9.3 mg, 10.25%) as a yellow solid. MS (ESI) calcd. for C₂₁H₂₆N₄O₆, 430.19 m/z, found 431.15 [M+H]⁺.

¹H NMR (400 MHz, DMSO-d₆) δ (ppm): 12.17 (s, 1H), 11.71 (s, 1H), 10.53 (s, 1H), 10.40 (s, 1H), 7.51 (t, J=8.4 Hz, 1H), 6.95 (d, J=7.5 Hz, 1H), 6.72-6.55 (m, 2H), 6.32 (s, 1H), 4.99 (s, 1H), 4.82 (s, 2H), 3.57 (q, J=6.7 Hz, 1H), 3.09-3.00 (m, 1H), 2.33-2.28 (m, 1H), 2.04-1.95 (m, 1H), 1.93-1.81 (m, 1H), 1.78-1.69 (m, 2H), 1.58 (s, 1H), 1.03-0.87 (m, 6H).

### Example 4.3: Synthesis of Compound 32: (1R,3S)-3-{5-[(2R)-2-(2-formyl-3-hydroxyphenoxy) propanamido]-2H-pyrazol-3-yl}cyclopentyl N-isopropylcarbamate

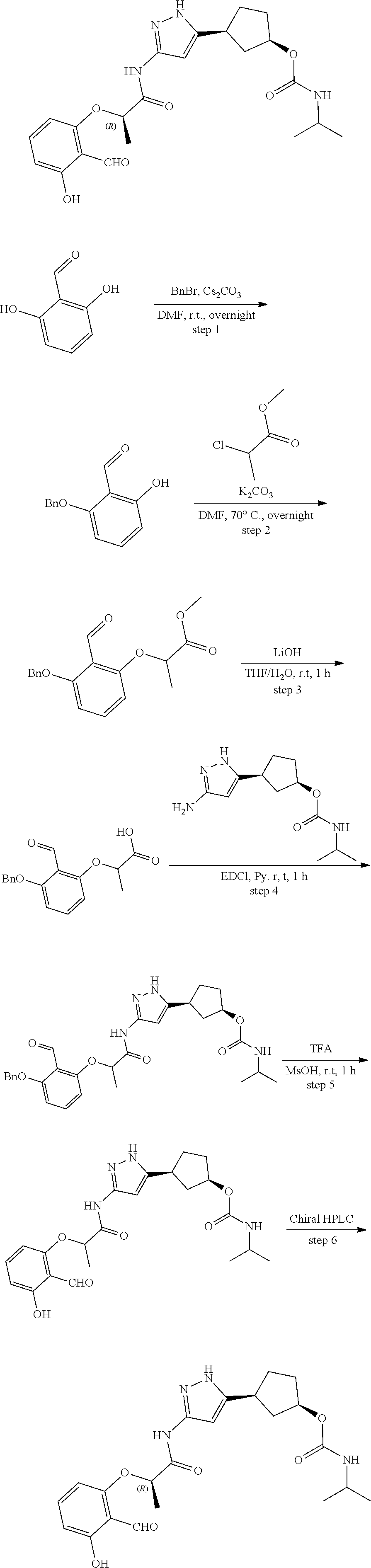

### Step 1: Synthesis of 2-(benzyloxy)-6-hydroxybenzaldehyde

To a solution of 2,6-dihydroxybenzaldehyde (10.0 g, 72.400 mmol, 1 equiv) and Cs₂CO₃ (11.8 g, 36.200 mmol, 0.5 equiv) in DMF (100 mL) was added benzyl bromide (24.7 g, 144.800 mmol, 2 equiv) dropwise at 0° C. and stirred at rt overnight. LCMS showed the reaction was completed. The resulting mixture was diluted with EA (300 mL). The resulting mixture was washed with water (150 mL×3) and brine (150 mL), dried over anhydrous Na₂SO₄. After filtration, the filtrate was concentrated under reduced pressure. The residue was purified by silica gel column chromatography, eluted with PE/EA (0-40%) to afford 2-(benzyloxy)-6-hydroxybenzaldehyde (13.5 g, 73.96%) as light yellow solid. MS (ESI) calcd. for C₁₄H₁₂O₃, 228.247 m/z, found: 227.05 [M−H]⁻.

### Step 2: Synthesis of methyl 2-[3-(benzyloxy)-2-formylphenoxy]propanoate

2-(Benzyloxy)-6-hydroxybenzaldehyde (500.0 mg, 2.191 mmol, 1 equiv), K₂CO₃ (756.8 mg, 5.477 mmol, 2.5 equiv) and DMF (5 mL) were added to a 25 mL round-bottom flask with a stir bar and stirred at rt for 15 min. To the above mixture was added methyl 2-chloropropanoate (536.9 mg, 4.382 mmol, 2 equiv) dropwise and the resulting mixture was stirred for additional overnight at 70° C. LCMS showed the reaction was completed. The resulting mixture was diluted with EA (25 mL). The resulting mixture was washed with water (15 mL×3) and brine (30 mL), dried over anhydrous Na₂SO₄. After filtration, the filtrate was concentrated under reduced pressure. The residue was purified by silica gel column chromatography, eluted PE/EA (0-30%) to afford methyl 2-[3-(benzyloxy)-2-formylphenoxy]propanoate (468.0 mg, 66.20%) as light yellow solid. MS (ESI) calcd. for C₁₈H₁₈O₅, 314.337 m/z, found: 315.05 [M+H]⁺.

### Step 3: Synthesis of 2-[3-(benzyloxy)-2-formylphenoxy]propanoic acid

A solution of methyl 2-[3-(benzyloxy)-2-formylphenoxy]propanoate (468.0 mg, 1.489 mmol, 1 equiv), LiOH (53.4 mg, 2.234 mmol, 1.5 equiv) and H₂O (1 mL) in THF (5 mL) was stirred for 1 h at rt. The mixture was acidified to pH 4 with HCl (0.5 mol/L). The resulting mixture was extracted with EA (30 mL). The resulting mixture was washed with water (15 mL×3) and brine (10 mL), dried over anhydrous Na₂SO₄. After filtration, the filtrate was concentrated under reduced pressure to afford 2-[3-(benzyloxy)-2-formylphenoxy]propanoic acid (408.0 mg, 83.50%) as a brown oil. MS (ESI) calcd. for C₁₇H₁₆O₅, 300.31 m/z, found: 301.05 [M+H]⁺.

### Step 4: Synthesis of (1R,3S)-3-(5-{2-[3-(benzyloxy)-2-formylphenoxy]propanamido}-2H-pyrazol-3-yl)cyclopentyl N-isopropylcarbamate

To a solution of 2-[3-(benzyloxy)-2-formylphenoxy]propanoic acid (170.0 mg, 0.566 mmol, 1 equiv) in pyridine (2 mL) were added (1R,3S)-3-(5-amino-2H-pyrazol-3-yl)cyclopentyl N-isopropylcarbamate (114.2 mg, 0.453 mmol, 0.8 equiv) and EDCI (162.7 mg, 0.849 mmol, 1.5 equiv). After stirring for 1 h at rt, LCMS showed the reaction was completed. The resulting mixture was diluted with EA (10 mL). The resulting mixture was washed with water (10 mL×3) and brine (10 mL), dried over anhydrous Na₂SO₄. After filtration, the filtrate was concentrated under reduced pressure. The residue was purified by Prep-TLC (PE/EA=1:1) to afford (1R,3S)-3-(5-{2-[3-(benzyloxy)-2-formylphenoxy]propanamido}-2H-pyrazol-3-yl)cyclopentyl N-isopropyl-carbamate (70.0 mg, 16.40%) as light brown oil. MS (ESI) calcd. for C₂₉H₃₄N₄O₆, 534.613 m/z, found 535.50 [M+H]⁺.

### Step 5: Synthesis of (1R,3S)-3-{5-[2-(2-formyl-3-hydroxyphenoxy)propanamido]-2H-pyrazol-3-yl}cyclopentyl N-isopropylcarbamate

A solution of (1R,3S)-3-(5-{2-[3-(benzyloxy)-2-formylphenoxy]propanamido}-2H-pyrazol-3-yl)cyclopentyl N-isopropylcarbamate (70.0 mg, 0.131 mmol, 1 equiv) in TFA (0.5 mL) and MsOH (0.1 mL) was stirred for 1 h at rt. The mixture was purified by Prep-HPLC under the condition: Column: Xselect CSH OBD Column, 30×150 mm, 5 um; Mobile Phase A: Water (0.1% FA), Mobile Phase B: ACN; Flow rate: 60 mL/min; Gradient: 30% B to 52% B in 10 min; Wave Length: 254/220 nm; RT1 (min): 9 to afford (1R,3S)-3-{5-[2-(2-formyl-3-hydroxyphenoxy)propanamido]-2H-pyrazol-3-yl}cyclopentyl N-isopropylcarbamate (2.5 mg, 4.29%) as a white solid. MS (ESI) calcd. for C₂₂H₂₈N₄O₆, 444.48 m/z, found 445.20 [M+H]⁺.

¹H NMR (400 MHz, DMSO-d₆) δ 12.17 (s, 1H), 11.73 (s, 1H), 10.65 (s, 1H), 10.40 (s, 1H), 7.49 (t, J=8.4 Hz, 1H), 6.94 (d, J=7.7 Hz, 1H), 6.54 (d, J=8.5 Hz, 1H), 6.45 (d, J=8.4 Hz, 1H), 6.30 (s, 1H), 5.03 (q, J=6.6 Hz, 1H), 4.98 (s, 1H), 3.58 (s, 1H), 3.04 (d, J=9.8 Hz, 1H), 2.43 (s, 1H), 2.31 (s, 1H), 1.99 (d, J=9.2 Hz, 1H), 1.88 (td, J=9.1, 8.2, 4.8 Hz, 1H), 1.68 (d, J=9.7 Hz, 1H), 1.57 (d, J=6.6 Hz, 3H), 1.02 (d, J=6.6 Hz, 6H).

### Step 6: Synthesis of (1R,3S)-3-{5-[(2R)-2-(2-formyl-3-hydroxyphenoxy)propanamido]-2H-pyrazol-3-yl}cyclopentyl N-isopropylcarbamate

(1R,3S)-3-{5-[2-(2-formyl-3-hydroxyphenoxy)propanamido]-2H-pyrazol-3-yl}cyclopentyl N-isopropylcarbamate was separated by Chiral HPLC under the following conditions: Column: CHIRALPAKID3; Mobile Phase: (Hex/DCM=3:1) (0.1% DEA):EtOH=70:30; Flow rate: 1.0 mL/min; Gradient: isocratic; Injection Volume: 3 μL mL to afford (1R,3S)-3-{5-[(2R)-2-(2-formyl-3-hydroxyphenoxy)propanamido]-2H-pyrazol-3-yl}cyclopentyl N-isopropylcarbamate (1.2 mg, 23.90%) as a light yellow solid. MS (ESI) calcd. for C₂₂H₂₈N₄O₆, 444.48 m/z, found 445.25 [M+H]^{+ 1}H NMR (400 MHz, DMSO-d₆) δ 12.17 (s, 1H), 11.72 (s, 1H), 10.64 (s, 1H), 10.40 (s, 1H), 7.49 (t, J=8.4 Hz, 1H), 6.94 (s, 1H), 6.54 (d, J=8.4 Hz, 1H), 6.45 (d, J=8.4 Hz, 1H), 6.30 (s, 1H), 5.03 (d, J=6.7 Hz, 1H), 4.98 (s, 2H), 3.53 (s, 1H), 3.03 (s, 1H), 1.98 (s, 1H), 1.87 (s, 1H), 1.70 (d, J=12.1 Hz, 2H), 1.57 (d, J=6.6 Hz, 3H), 1.24 (s, 1H), 1.02 (d, J=6.5 Hz, 6H).

### Example 4.4: Synthesis of Compound 36: (1R,3S)-3-(5-{2-[(2-formyl-3-hydroxyphenyl)methoxy]acetamido}-2H-pyrazol-3-yl)cyclopentyl N-isopropylcarbamate; trifluoroacetic acid

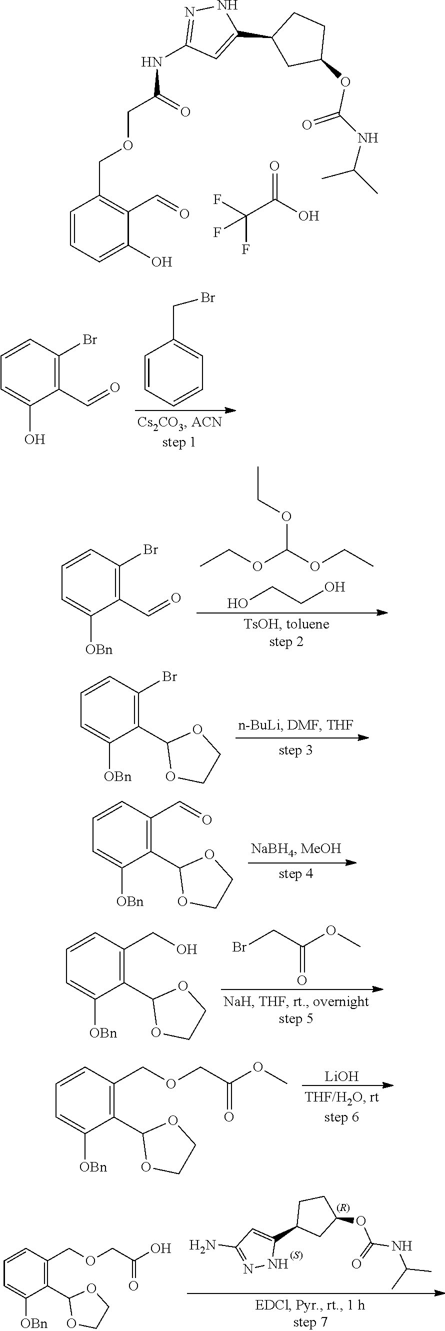

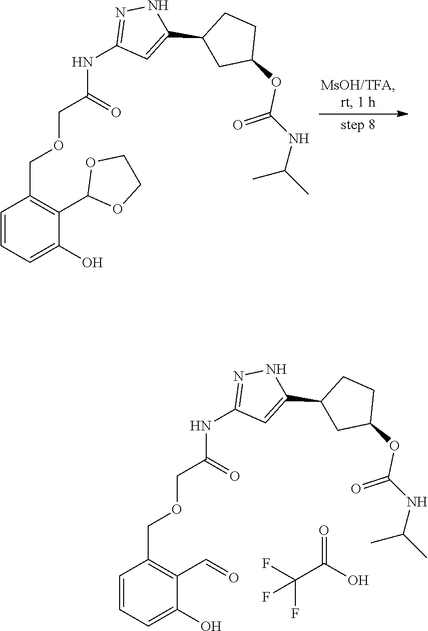

### Step 1: Synthesis of 2-(benzyloxy)-6-bromobenzaldehyde

To a solution of 2-bromo-6-hydroxybenzaldehyde (5.0 g, 24.873 mmol, 1 equiv) in acetonitrile (80 mL) was added benzyl bromide (6.38 g, 37.309 mmol, 1.5 equiv) and Cs₂CO₃ (16.21 g, 49.746 mmol, 2.0 equiv). The resulting mixture was stirred overnight at rt. LCMS showed the desired product was generated. The reaction was quenched by the addition of saturated NH₄Cl aqueous at 0° C. The resulting mixture was extracted with EA (3×200 mL). The combined organic layer was washed with brine (3×200 mL), dried over anhydrous Na₂SO₄. The solvent was concentrated under reduced pressure. The residue was purified by silica gel column chromatography, eluted with PE/EA (5:1) to afford 2-(benzyloxy)-6-bromobenzaldehyde as a white solid (7.0 g, 91% yield). MS (ESI) calcd. for C₁₄H₁₁BrO₂, 289.99 m/z, found: 291.01 [M+H]⁺

### Step 2: Synthesis of 2-(2-(benzyloxy)-6-bromophenyl)-1,3-dioxolane

To a colorless solution of 2-(benzyloxy)-6-bromobenzaldehyde (5 g, 17.174 mmol, 1 equiv) in toluene (70 mL, 657.905 mmol, 38.31 equiv) was added TsOH (0.30 g, 1.717 mmol, 0.1 equiv), ethylene glycol (5.33 g, 85.870 mmol, 5 equiv) and triethyl orthoformate (7.64 g, 51.522 mmol, 3 equiv) at rt. The obtained colorless solution was stirred at r.t for 10 min giving a colorless solution. The resulting solution was stirred overnight at 90° C. After cooled down to r.t, the reaction was quenched by the addition of NH₄Cl aq. (50 mL) at 0° C. The resulting mixture was extracted with EA (50 mL×3). The combined organic layers were washed with brine (100 mL×3), dried over anhydrous Na₂SO₄. After filtration, the filtrate was concentrated under reduced pressure. The residue was purified by silica gel column (0-7% PE/EA) giving 2-[2-(benzyloxy)-6-bromophenyl]-1,3-dioxolane (4.97 g, 86.34% yield) as a light yellow solid. MS (ESI) calcd. for C₁₆H₁₅BrO₃, 334.02 m/z, found: 335.00 [M+H]⁺

### Step 3: Synthesis of 3-(benzyloxy)-2-(1,3-dioxolan-2-yl)benzaldehyde

To a solution of 2-[2-(benzyloxy)-6-bromophenyl]-1,3-dioxolane (3 g, 8.950 mmol, 1 equiv) in THF (30 mL) at −78° C. under nitrogen atmosphere was added a solution of n-BuLi (4.30 mL, 10.740 mmol, 1.2 equiv) 2.5 M in hexanes and was stirred at −78° C. under N₂ for 30 min. To the above mixture was added DMF (15 mL) dropwise at −78° C. The resulting mixture was stirred for additional 30 min at −78° C. Desired product could be detected by LCMS. The reaction was quenched with sat. NH₄Cl (aq.) at −78° C. The reaction was extracted with EA (50 mL×3). The combined organic extracts were washed with brine (30 mL), dried over anhydrous Na₂SO₄. The residue was purified by silica gel column chromatography, eluted with PE/EA (0-55%) to afford 3-(benzyloxy)-2-(1,3-dioxolan-2-yl)benzaldehyde (2.15 g, 80.27%) as a yellow oil. MS (ESI) calcd. for C₁₇H₁₆O₄, 284.10 m/z, found 285.10 [M+H]⁺

### Step 4: Synthesis of (3-(benzyloxy)-2-(1,3-dioxolan-2-yl)phenyl)methanol

A solution of 3-(benzyloxy)-2-(1,3-dioxolan-2-yl)benzaldehyde (1.5 g, 5.276 mmol, 1 equiv) and NaBH₄ (99.79 mg, 2.638 mmol, 0.5 equiv) in MeOH (15 mL) was stirred at 25° C. for 8 h. The reaction was quenched with sat. NaHCO₃. The resulting mixture was extracted with EA (3×50 ml). The combined organic layers were washed with H₂O (2×30 ml), dried over anhydrous Na₂SO₄. After filtration, the filtrate was concentrated under reduced pressure. The residue was purified by silica gel column with PE/EA (0-70%) to afford [3-(benzyloxy)-2-(1,3-dioxolan-2-yl)phenyl]methanol (750 mg, 45.18%) as a white oil. MS (ESI) calcd. for C₁₇H₁₈O₄, 286.12 m/z, found 287.10 [M+H]⁺.

### Step 5: Synthesis of methyl 2-{[3-(benzyloxy)-2-(1,3-dioxolan-2-yl)phenyl]methoxy}acetate

To a stirred solution of [3-(benzyloxy)-2-(1,3-dioxolan-2-yl)phenyl]methanol (300.0 mg, 1.048 mmol, 1 equiv) in THF (10 mL, 123.428 mmol) was added NaH (75.4 mg, 3.144 mmol, 3 equiv) in portions at 0° C. under N₂ atmosphere. The resulting mixture was stirred for 10 min at 0° C. under N₂ atmosphere. To the above mixture was added methyl 2-bromoacetate (480.8 mg, 3.144 mmol, 3 equiv) dropwise over 5 min at 0° C. The resulting mixture was stirred for additional 3 h at room temperature. The reaction was quenched with H₂O at room temperature. The resulting mixture was extracted with EA (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (45%) to afford methyl 2-{[3-(benzyloxy)-2-(1,3-dioxolan-2-yl)phenyl]methoxy}acetate (230.0 mg, 61.25%) as a colorless oil. MS (ESI) calcd. for C₂₀H₂₂O₆, 358.14 m/z, found 359.15 [M+H]⁺.

### Step 6: Synthesis of {[3-(benzyloxy)-2-(1,3-dioxolan-2-yl)phenyl]methoxy}acetic acid

To a stirred solution of methyl 2-{[3-(benzyloxy)-2-(1,3-dioxolan-2-yl)phenyl]methoxy}acetate (230.0 mg, 0.642 mmol, 1 equiv) in THF (2 mL) and H₂O (2 mL) was added LiOH (46.1 mg, 1.926 mmol, 3 equiv) at room temperature. The resulting mixture was stirred for 2 h at room temperature. The mixture was acidified to pH 7-8 with citric acid. The resulting mixture was extracted with EA (3×10 mL). The combined organic layers were washed with brine (3×10 mL), dried over anhydrous Na₂SO₄. After filtration, the filtrate was concentrated under reduced pressure to afford {[3-(benzyloxy)-2-(1,3-dioxolan-2-yl)phenyl]methoxy}acetic acid (130.0 mg, 58.82%) as a yellow solid. MS (ESI) calcd. for C₁₉H₂₀O₆, 344.13 m/z, found 345.10 [M+H]⁺.

### Step 7: Synthesis of (1R,3S)-3-[5-(2-{[3-(benzyloxy)-2-(1,3-dioxolan-2-yl)phenyl]methoxy}acetamido)-2H-pyrazol-3-yl]cyclopentyl N-isopropylcarbamate

To a stirred solution of {[3-(benzyloxy)-2-(1,3-dioxolan-2-yl)phenyl]methoxy}acetic acid (130.0 mg, 0.378 mmol, 1 equiv) and (1R,3S)-3-(5-amino-2H-pyrazol-3-yl)cyclopentyl N-isopropylcarbamate (95.3 mg, 0.378 mmol, 1 equiv) in pyridine (5 mL) was added EDCI (289.5 mg, 1.512 mmol, 4 equiv) at room temperature. The resulting mixture was stirred for 1 h at room temperature, then diluted with water. The resulting mixture was extracted with EA (3×10 mL). The combined organic layers were washed with brine (3×10 mL), dried over anhydrous Na₂SO₄. After filtration, the filtrate was concentrated under reduced pressure. The residue was purified by reversed-phase flash chromatography with the following conditions: column, C18; mobile phase, ACN/H₂O (0.5% NH₄HCO₃ modifier), 5% to 90% gradient in 40 min; detector, UV 254 nm. This resulted in (1R,3S)-3-[5-(2-{[3-(benzyloxy)-2-(1,3-dioxolan-2-yl)phenyl]methoxy}acetamido)-2H-pyrazol-3-yl]cyclopentyl N-isopropylcarbamate (90.0 mg, 41.20%) as a yellow solid. MS (ESI) calcd. for C₃₁H₃₈N₄O₇, 578.27 m/z, found 579.30 [M+H]⁺.

### Step 8: Synthesis of (1R,3S)-3-(5-{2-[(2-formyl-3-hydroxyphenyl)methoxy]acetamido}-2H-pyrazol-3-yl)cyclopentyl N-isopropylcarbamate; trifluoroacetic acid

To a stirred solution of (1R,3S)-3-[5-(2-{[3-(benzyloxy)-2-(1,3-dioxolan-2-yl)phenyl]methoxy}acetamido)-2H-pyrazol-3-yl]cyclopentyl N-isopropylcarbamate (60.0 mg, 0.104 mmol, 1 equiv) in trifluoroacetic acid (3 mL, 0.031 mmol, 0.30 equiv) was added methanesulfonic acid (1 mL, 0.010 mmol, 0.10 equiv) dropwise at room temperature. The resulting mixture was stirred for 1 h at room temperature. The mixture was neutralized to pH 7-8 with Na₂CO₃ (aq.). The resulting mixture was extracted with DCM (3×10 mL). The combined organic layers were washed with brine (3×10 mL), dried over anhydrous Na₂SO₄. After filtration, the filtrate was concentrated under reduced pressure. The crude product was purified by Prep-HPLC with the following conditions (Column: Xselect CSH OBD Column, 30×150 mm, 5 um; Mobile Phase A: Water (0.05% TFA), Mobile Phase B: ACN; Flow rate: 60 mL/min; Gradient: 26% B to 50% B in 10 min; Wave Length: 254 nm/220 nm nm; RT1 (min): 8) to afford (1R,3S)-3-(5-{2-[(2-formyl-3-hydroxyphenyl)methoxy]acetamido}-2H-pyrazol-3-yl)cyclopentyl N-isopropylcarbamate; trifluoroacetic acid (9.8 mg, 15.84%) as an off-white solid. MS (ESI) calcd. for C₂₂H₂₈N₄O₆, 444.20 m/z, found 445.20 [M+H]⁺. ¹H NMR (400 MHz, DMSO-d₆) δ (ppm): 11.26 (br s, 1H), 10.49 (s, 1H), 10.13 (s, 1H), 7.51-7.55 (m, 1H), 7.08-7.10 (m, 1H), 6.93-6.96 (m, 2H), 6.32 (s, 1H), 4.99-5.00 (m, 1H), 4.87 (s, 2H), 4.16 (s, 2H), 3.53-3.60 (m, 1H), 3.02-3.07 (m, 1H), 2.45-2.47 (m, 1H), 1.98-2.07 (m, 1H), 1.84-1.93 (m, 1H), 1.67-1.73 (m, 2H), 1.56-1.64 (m, 1H), 1.02-1.04 (m, 6H). ¹⁹F NMR (376 MHz, DMSO-d₆) δ (ppm): −74.87.

### Example 4.5: Synthesis of Compound 43: (1R,3S)-3-{5-[2-(4-fluoro-2-formyl-3-hydroxyphenoxy) acetamido]-2H-pyrazol-3-yl}cyclopentyl N-isopropylcarbamate; trifluoroacetic acid

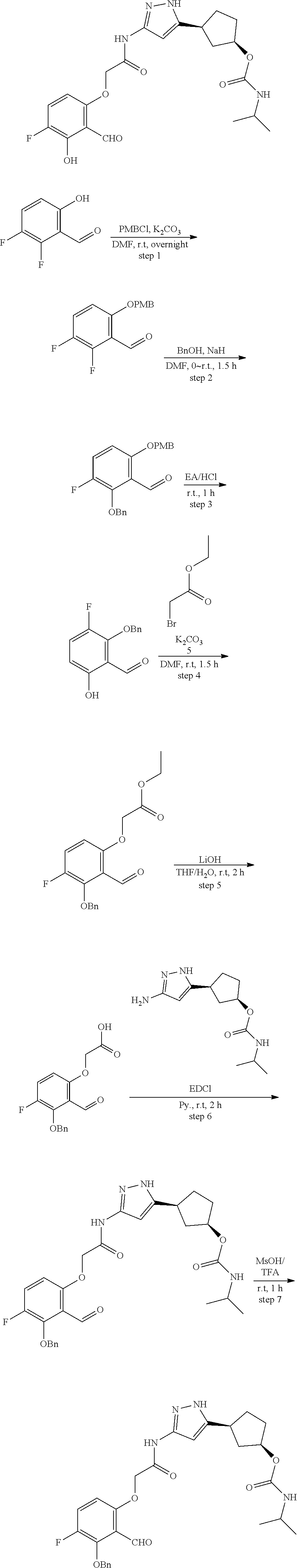

### Step 1: Synthesis of 2,3-difluoro-6-((4-methoxybenzyl)oxy)benzaldehyde

To a stirred solution of 2,3-difluoro-6-hydroxybenzaldehyde (500.0 mg, 3.162 mmol, 1 equiv) in DMF (10 mL) was added Cs₂CO₃ (2576.0 mg, 7.905 mmol, 2.5 equiv) at room temperature. The resulting mixture was stirred for 30 min at room temperature. To the above mixture was added PMBCl (643.9 mg, 4.111 mmol, 1.3 equiv) dropwise at 0° C. The resulting mixture was stirred overnight at room temperature. The reaction was quenched with water at room temperature. The resulting mixture was extracted with EtOAc (3×15 mL). The combined organic layers were washed with a saturated solution of NaCl (3×10 mL), dried over anhydrous Na₂SO₄. The resulting mixture was concentrated under vacuum. The residue was purified by silica gel column chromatography, eluted with EA/PE (1:1) to afford 2,3-difluoro-6-[(4-methoxyphenyl) methoxy]benzaldehyde (700.0 mg, 79.55%) as a yellow solid. MS (ESI) calcd. for C₁₅H₁₂F₂O₃, 278.20 m/z, found 279.10 [M+H]⁺.

### Step 2: Synthesis of 2-(benzyloxy)-3-fluoro-6-((4-methoxybenzyl)oxy)benzaldehyde

To a stirred solution of benzyl alcohol (505.2 mg, 4.672 mmol, 2 equiv) in DMF (10 mL) was added NaH (140 mg, 3.504 mmol, 1.5 equiv, 60% suspension in oil) at 0° C. The resulting mixture was stirred for 1 h at 0° C. under nitrogen atmosphere. To the above mixture was added 2,3-difluoro-6-[(4-methoxyphenyl)methoxy]benzaldehyde (650.0 mg, 2.336 mmol, 1 equiv) at 0° C. The resulting mixture was stirred for additional 1 h at room temperature. The reaction was quenched with sat. NH₄Cl (aq.) at 0° C. The resulting mixture was extracted with EtOAc (3×10 mL). The combined organic layers were washed with NaCl (aq. 3×10 mL), dried over anhydrous Na₂SO₄. The resulting mixture was concentrated under vacuum. The residue was purified by silica gel column chromatography, eluted with EA/PE (1:1) to afford 2-(benzyloxy)-3-fluoro-6-((4-methoxybenzyl)oxy)benzaldehyde (300.6 mg, 35.05%) as a colorless oil. MS (ESI) calcd. for C₂₂H₁₉FO₄, 366.40 m/z, found 367.20 [M+H]⁺.

### Step 3: Synthesis of 6-(benzyloxy)-3-fluoro-2-hydroxybenzaldehyde

To a stirred solution of 2,3-difluoro-6-[(4-methoxyphenyl)methoxy]benzaldehyde (240.0 mg, 0.863 mmol, 1 equiv) in EA (1 mL) was added 3 mL HCl (aq. 4M in EA) dropwise at room temperature. The resulting mixture was stirred for 1 h at room temperature. The resulting mixture was concentrated under vacuum. The crude product was used in the next step directly without further purification. MS (ESI) calcd. for C₁₄H₁₁FO₃, 246.20 m/z, found 245.30 [M−H]⁻.

### Step 4: Synthesis of ethyl 2-[3-(benzyloxy)-4-fluoro-2-formylphenoxy]acetate

To a stirred solution of 2-(benzyloxy)-3-fluoro-6-hydroxybenzaldehyde (140.0 mg, 0.569 mmol, 1 equiv) in DMF (3 mL) was added K2CO₃ (196.4 mg, 1.422 mmol, 2.5 equiv) at room temperature. The resulting mixture was stirred for 0.5 h at room temperature. To the above mixture was added ethyl bromoacetate (113.9 mg, 0.683 mmol, 1.20 equiv) at 0° C. The resulting mixture was stirred for additional 1 h at room temperature. The reaction was quenched with water at room temperature. The resulting mixture was extracted with EtOAc (3×5 mL). The combined organic layers were washed with NaCl (aq. 3×5 mL), dried over anhydrous Na₂SO₄. After filtration, the filtrate was concentrated under reduced pressure. The residue was purified by silica gel column chromatography, eluted with EA/PE (1:1) to afford ethyl 2-[3-(benzyloxy)-4-fluoro-2-formylphenoxy]acetate (170.0 mg, 89.97%) as a light yellow oil. MS (ESI) calcd. for C₁₈H₁₇FO₅, 332.40 m/z, found 333.10 [M+H]⁺.

### Step 5: Synthesis of 3-(benzyloxy)-4-fluoro-2-formylphenoxyacetic acid

To a stirred solution of ethyl 2-[3-(benzyloxy)-4-fluoro-2-formylphenoxy]acetate (170.0 mg, 0.512 mmol, 1 equiv) in THF (2.5 mL) and H₂O (0.5 mL) was added lithium hydroxide (18.4 mg, 0.768 mmol, 1.5 equiv) at room temperature. The resulting mixture was stirred for 2 h at room temperature. The resulting mixture was concentrated under vacuum. The residue was dissolved in water (2 mL). The mixture was acidified to pH 7 with HCl (aq. 2M in H₂O). The resulting mixture was concentrated under vacuum. The crude product was used in the next step directly without further purification. MS (ESI) calcd. for C₁₆H₁₃FO₅, 304.30 m/z, found 303.10 [M−H]⁻.

### Step 6: Synthesis of (1R,3S)-3-(5-{2-[3-(benzyloxy)-4-fluoro-2-formylphenoxy]acetamido}-2H-pyrazol-3-yl)cyclopentyl N-isopropylcarbamate

To a stirred solution of 3-(benzyloxy)-4-fluoro-2-formylphenoxyacetic acid (80.0 mg, 0.263 mmol, 2 equiv) and (1R,3S)-3-(5-amino-2H-pyrazol-3-yl)cyclopentyl N-isopropylcarbamate (33.2 mg, 0.132 mmol, 1 equiv) in pyridine (1 mL) was added EDCI (37.8 mg, 0.197 mmol, 1.5 equiv) at room temperature under nitrogen atmosphere. The resulting mixture was stirred for additional 2 h at room temperature. The reaction was quenched with water at room temperature. The resulting mixture was extracted with EtOAc (3×2 mL). The combined organic layers were washed with NaCl (aq. 3×2 mL), dried over anhydrous Na₂SO₄. After filtration, the filtrate was concentrated under reduced pressure. The residue was purified by Prep-TLC (MeOH/CH₂Cl₂ 1:5) to afford (1R,3S)-3-(5-{2-[3-(benzyloxy)-4-fluoro-2-formylphenoxy]acetamido}-2H-pyrazol-3-yl)cyclopentyl N-isopropylcarbamate (25.0 mg, 35.31%) as a light yellow solid. MS (ESI) calcd. for C₂₈H₃₁FN₄O₆, 538.50 m/z, found 539.20 [M+H]⁺.

### Step 7: Synthesis of (1R,3S)-3-{5-[2-(4-fluoro-2-formyl-3-hydroxyphenoxy)acetamido]-2H-pyrazol-3-yl}cyclopentyl N-isopropylcarbamate; trifluoroacetic acid

A stirred solution of (1R,3S)-3-(5-{2-[3-(benzyloxy)-4-fluoro-2-formylphenoxy]acetamido}-2H-pyrazol-3-yl)cyclopentyl N-isopropylcarbamate (25.0 mg, 0.046 mmol, 1 equiv) in TFA (1 mL) and methanesulfonic acid (1 mL) was stirred for 1 h at room temperature. The crude product was purified by Prep-HPLC with the following conditions (Column: Xselect CSH Prep C18 OBD Column, 30×150 mm, 5 um; Mobile Phase A: Water (0.05% TFA), Mobile Phase B: ACN; Flow rate: 60 mL/min; Gradient: 29% B to 51% B in 10 min; Wave Length: 254/220 nm) to afford (1R,3S)-3-{5-[2-(4-fluoro-2-formyl-3-hydroxyphenoxy)acetamido]-2H-pyrazol-3-yl}cyclopentyl N-isopropylcarbamate; trifluoroacetic acid (1.2 mg, 4.16%) as a grey solid. MS (ESI) calcd. for C₂₃H₂₆F₄N₄O₈, 448.50 m/z, found 449.20 [M+H]⁺. ¹H NMR (400 MHz, DMSO-d₆) δ 11.68 (s, 1H), 10.51 (s, 1H), 10.39 (d, J=1.6 Hz, 1H), 7.48-7.58 (m, 1H), 6.90-7.00 (m, 1H), 6.47-6.54 (m, 1H), 6.31 (s, 1H), 4.99 (s, 1H), 4.82 (s, 2H), 3.00-3.09 (m, 1H), 2.45 (s, 1H), 1.95-2.04 (m, 1H), 1.81-1.93 (m, 1H), 1.70 (t, J=10.7 Hz, 2H), 1.58 (s, 1H), 0.99-1.06 (m, 6H). ⁹F NMR (377 MHz, DMSO-d6) δ −146.32.

### Example 4.6: Synthesis of Compound 46: (1R,3S)-3-{5-[2-(3-acetamido-2-formylphenoxy) acetamido]-2H-pyrazol-3-yl}cyclopentyl N-isopropylcarbamate

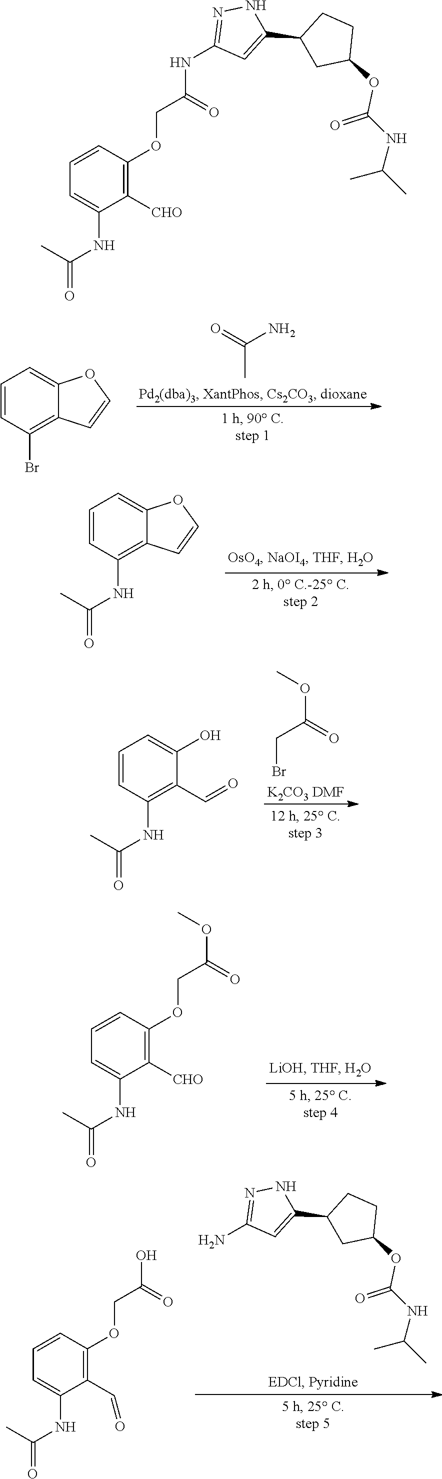

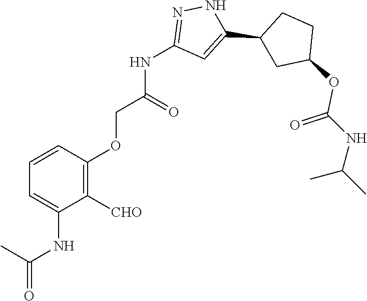 [SMILES: CC(=O)Nc1cccc(OCC(=O)Nc2cc([C@H]3CC[C@@H](OC(=O)NC(C)C)C3)[nH]n2)c1C=O]

### Step 1: Synthesis of N-(1-benzofuran-4-yl)acetamide

A solution of 4-bromo-1-benzofuran (700 mg, 3.553 mmol, 1 equiv), acetamide (419.7 mg, 7.106 mmol, 2 equiv), Cs₂CO₃ (2.89 g, 8.883 mmol, 2.5 equiv), XantPhos (411.2 mg, 0.711 mmol, 0.2 equiv) and Pd₂(dba)₃ (325.3 mg, 0.355 mmol, 0.1 equiv) in dioxane (5 mL) was stirred for 1 h at 90° C. under nitrogen atmosphere. The mixture was diluted with EA (10 mL). The resulting mixture was extracted with H₂O (10 mL×3), dried over anhydrous Na₂SO₄. The resulting mixture was concentrated under reduced pressure. The residue was purified by silica gel column chromatography, eluted with EA:PE (3:1) to afford N-(1-benzofuran-4-yl)acetamide (590 mg, 90.25%) as a yellow solid. MS (ESI) calcd. for C₁₀H₉NO₂, 175.06 m/z, found 176.10 [M+H]⁺.

### Step 2: Synthesis of N-(2-formyl-3-hydroxyphenyl)acetamide

A solution of N-(1-benzofuran-4-yl)acetamide (570 mg, 3.254 mmol, 1 equiv) in THF (2.5 mL, 6.171 mmol) and H₂O (2.5 mL, 27.755 mmol) was treated with OsO₄ (4.14 mL, 3.254 mmol, 1 equiv) for 1 h at 0° C. under nitrogen atmosphere followed by the addition of NaIO₄ (4.21 g, 19.524 mmol, 6 equiv) in portions at 25° C. The mixture was stirred for 1 h at 25° C. The mixture was diluted with EA (20 mL). The resulting mixture was extracted with H₂O (20 mL×3), dried over anhydrous Na₂SO₄. The resulting mixture was concentrated under reduced pressure. The crude product mixture was used in the next step directly without further purification. MS (ESI) calcd. for C₉H₉NO₃, 179.10 m/z, found 180.10 [M+H]⁺.

### Step 3: Synthesis of methyl 2-(3-acetamido-2-formylphenoxy)acetate

A solution of N-(2-formyl-3-hydroxyphenyl)acetamide (550 mg, 3.070 mmol, 1 equiv) in DMF (5 mL) was treated with K₂CO₃ (636.4 mg, 4.605 mmol, 1.5 equiv) for 30 mins at 25° C. followed by the addition of methyl 2-bromoacetate (563.5 mg, 3.684 mmol, 1.2 equiv) dropwise at 25° C. The mixture was stirred for 12 h at 25° C. The mixture was diluted with EA (10 mL). The resulting mixture was washed with H₂O (10 mL×3), dried over anhydrous Na₂SO₄. The resulting mixture was concentrated under reduced pressure. The residue was purified by silica gel column chromatography, eluted with EA:PE (1:1) to afford methyl 2-(3-acetamido-2-formyl phenoxy)acetate (220 mg, 27.47%) as a white solid. MS (ESI) calcd. for C₁₂H₁₃NO₅, 251.20 m/z, found 252.20 [M+H]⁺.

### Step 4: Synthesis of 3-acetamido-2-formylphenoxyacetic acid

A solution of methyl 2-(3-acetamido-2-formylphenoxy)acetate (210 mg, 0.836 mmol, 1 equiv) and LiOH (24.1 mg, 1.003 mmol, 1.2 equiv) in THF (5 mL, 61.714 mmol) and H₂O (1 mL, 55.509 mmol) was stirred for 12 h at 25° C. The resulting mixture was concentrated under reduced pressure. The mixture was neutralized to pH 7 with HCl (2 M). The mixture was diluted with EA (10 mL). The resulting mixture was washed with H₂O (10 mL×3), dried over anhydrous Na₂SO₄. After filtration, the filtrate was concentrated under reduced pressure. This resulted in 3-acetamido-2-formylphenoxyacetic acid (150 mg, 68.16%) as a white solid. MS (ESI) calcd. for C₁₁H₁₁NO₅, 237.20 m/z, found 238.20 [M+H]⁺.

### Step 5: Synthesis of (1R,3S)-3-{5-[2-(3-acetamido-2-formylphenoxy)acetamido]-2H-pyrazol-3-yl}cyclopentyl N-isopropylcarbamate

A solution of 3-acetamido-2-formylphenoxyacetic acid (135.38 mg, 0.571 mmol, 1.2 equiv) and (1R,3S)-3-(5-amino-2H-pyrazol-3-yl)cyclopentyl N-isopropylcarbamate (120 mg, 0.476 mmol, 1.00 equiv) and EDCI (109.4 mg, 0.571 mmol, 1.2 equiv) in pyridine (3 mL) was stirred for 12 h at 25° C. The mixture was diluted with EA (10 mL). The resulting mixture was washed with H₂O (10 mL×3), dried over anhydrous Na₂SO₄. The resulting mixture was concentrated under reduced pressure. The crude product was purified by Column: XSelect CSH C18 Column, 19*250 mm, 5 μm; Mobile Phase A: Water (0.1% FA), Mobile Phase B: ACN; Flow rate: 25 mL/min mL/min; Gradient: 31% B to 56% B in 10 min; Wave Length: 254 nm nm; RT1 (min): 9.82 to afford (1R,3S)-3-{5-[2-(3-acetamido-2-formylphenoxy)acetamido]-2H-pyrazol-3-yl}cyclopentyl N-isopropylcarbamate (14.1 mg, 6.22%) as a white solid. MS (ESI) calcd. for C₂₃H₂₉N₅O₆, 471.20 m/z, found 472.20 m/z [M+H]⁺. ¹H NMR (400 MHz, DMSO-d₆) δ (ppm) 12.16 (s, 1H), 11.41 (s, 1H), 10.58 (s, 1H), 10.51 (s, 1H), 8.09 (d, J=8.4 Hz, 1H), 7.60 (t, J=8.5 Hz, 1H), 6.95 (d, J=7.9 Hz, 1H), 6.79 (d, J=8.4 Hz, 1H), 6.32 (s, 1H), 4.99 (s, 1H), 4.87 (s, 2H), 3.57 (q, J=6.8 Hz, 1H), 3.04 (t, J=8.7 Hz, 1H), 2.51 (s, 1H), 2.17 (s, 3H), 2.04-1.95 (m, 1H), 1.93-1.84 (m, 1H), 1.79-1.69 (m, 2H), 1.58 (s, 1H), 1.02 (dd, J=6.7, 2.1 Hz, 6H).

### Example 4.7: Synthesis of Compound 47: 1R,3S)-3-(3-(2-(5-cyano-2-formyl-3-hydroxyphenoxy) acetamido)-1H-pyrazol-5-yl)cyclopentyl isopropylcarbamate

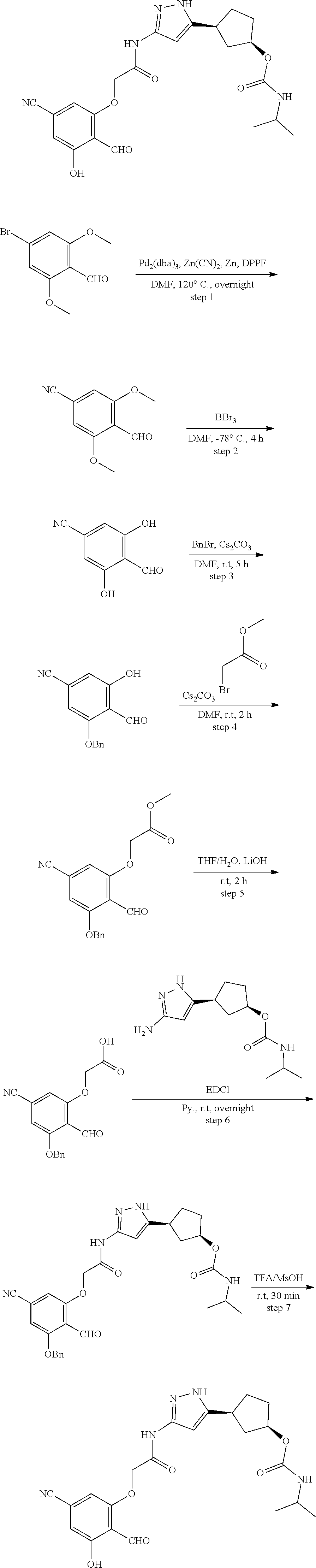

### Step 1: Synthesis of 4-formyl-3,5-dimethoxybenzonitrile

4-Bromo-2,6-dimethoxybenzaldehyde (3.0 g, 12.241 mmol, 1 equiv), Pd₂(dba)₃ (1.1 g, 1.224 mmol, 0.1 equiv), DPPF (1.4 g, 2.448 mmol, 0.2 equiv), Zn (0.4 g, 6.120 mmol, 0.5 equiv), Zn(CN)₂ (2.2 g, 18.361 mmol, 1.5 equiv) and DMF (40 mL) were added to a 100 mL reaction flask and stirred overnight at 120° C. under nitrogen atmosphere. The reaction mixture was then treated with H₂O (80 mL) and extracted with EA (40 mL×3), and the combined extracts washed with brine (40 mL), dried over anhydrous Na₂SO₄, filtered, and concentrated to dryness under vacuum. The residue was purified by silica gel column chromatography, eluted with PE/EA (0-27%) to afford 4-formyl-3,5-dimethoxybenzonitrile (3.0 g, 96.27%) as a brown solid. MS (ESI) calcd. For C₁₀H₉NO₃, 191.06 m/z, found 192.10 [M+H]⁺.

### Step 2: Synthesis of 4-formyl-3,5-dihydroxybenzonitrile

A solution of 4-formyl-3,5-dimethoxybenzonitrile (1.0 g, 5.231 mmol, 1 equiv) in DCM (20 mL) was treated with boron tribromide (15.7 mL, 15.690 mmol, 3.00 equiv, 1M in DCM) and stirred for 4 h at −78° C. under nitrogen atmosphere. The reaction mixture was then treated with NaHCO₃ (30 mL) extracted with DCM (30 mL×2), the combined extracts washed with brine (10 mL), dried over anhydrous Na₂SO₄, filtered, and concentrated to dryness under vacuum. The residue was purified by silica gel column chromatography, eluted with MeOH/DCM (0-10%) to afford 4-formyl-3,5-dihydroxybenzonitrile (435.0 mg, 50.98%) as a yellow solid. MS (ESI) calcd. For C₈H₅NO₃, 163.32 m/z, found 162.15 [M−H]⁻.

### Step 3: Synthesis of 3-(benzyloxy)-4-formyl-5-hydroxybenzonitrile

4-Formyl-3,5-dihydroxybenzonitrile (180.0 mg, 0.524 mmol, 1 equiv), and Cs₂CO₃ (216.2 mg, 1.324 mmol, 0.6 equiv) was dissolved in DCM (9 mL) and stirred for 0.5 h at rt. To the above mixture benzyl bromide (230.2 mg, 0.629 mmol, 1.2 equiv) was added at −78° C. and stirred for 5 h at rt under nitrogen atmosphere. The reaction mixture was then treated with a saturated solution of NaHCO₃ (20 mL) and extracted with DCM (10 mL×2), the combined extracts were washed with brine (10 mL), dried over anhydrous Na₂SO₄, filtered, and concentrated to dryness under vacuum. The residue was purified by silica gel column chromatography, eluted with PE/EA (0-30%) to afford 3-(benzyloxy)-4-formyl-5-hydroxybenzonitrile (120.0 mg, 90.39%) as a yellow solid. MS (ESI) calcd. For C₁₅H₁₁NO₃, 253.07 m/z, found 254.15 [M+H]⁺.

### Step 4: Synthesis of methyl 2-(3-(benzyloxy)-5-cyano-2-formylphenoxy)acetate

3-(Benzyloxy)-4-formyl-5-hydroxybenzonitrile (120.0 mg, 0.643 mmol, 1 equiv), and Cs₂CO₃ (192.2 mg, 0.592 mmol, 0.6 equiv) was dissolved in DMF (10 mL) and stirred for 0.5 h at rt. To the above methyl 2-bromoacetate (150.0 mg, 0.435 mmol, 1.1 equiv) was added and stirred for 2 h at rt. The reaction mixture was then treated with H₂O (20 mL) and extracted with EA (10 mL×3), the combined extracts washed with brine (10 mL), dried over anhydrous Na₂SO₄, filtered, and concentrated to dryness under vacuum. The residue was purified by silica gel column chromatography, eluted with PE/EA (0-40%) to afford methyl 2-[3-(benzyloxy)-5-cyano-2-formylphenoxy]acetate (120.0 mg, 93.42%) as a yellow oil. MS (ESI) calcd. For C₁₈H₁₅NO₅, 325.02 m/z, found 326.15 [M+H]⁺.

### Step 5: Synthesis of 2-(3-(benzyloxy)-5-cyano-2-formylphenoxy)acetic acid

A solution of methyl 2-[3-(benzyloxy)-5-cyano-2-formylphenoxy]acetate (100.0 mg, 0.307 mmol, 1 equiv) in THF (3 mL) and H₂O (1 mL) was treated with LiOH (18.4 mg, 0.767 mmol, 2.5 equiv), for 2 h at rt. The mixture was acidified to pH 6 with HCl (2 mol/L), the mixture was extracted with EA (5 mL×3), the combined extracts washed with brine (5 mL), dried over anhydrous Na₂SO₄, filtered, and concentrated to dryness under vacuum. The resulting mixture was concentrated under reduced pressure to afford methyl 2-[3-(benzyloxy)-5-cyano-2-formylphenoxy]acetate (80.0 mg, 80.0%) as a white solid. MS (ESI) calcd. For C₁₇H₁₃NO₅, 311.02 m/z, found 312.15 [M+H]⁺.

### Step 6: Synthesis of (1R,3S)-3-(3-(2-(3-(benzyloxy)-5-cyano-2-formylphenoxy)acetamido)-1H-pyrazol-5-yl)cyclopentyl isopropylcarbamate

A solution of 3-(benzyloxy)-5-cyano-2-formylphenoxyacetic acid (81.4 mg, 0.262 mmol, 1.2 equiv) in pyridine (1 mL) was treated with (1R,3S)-3-(5-amino-2H-pyrazol-3-yl)cyclopentyl N-isopropylcarbamate (55.0 mg, 0.218 mmol, 1.00 equiv), EDCI (62.7 mg, 0.327 mmol, 1.5 equiv) and stirred overnight at rt. The reaction mixture was then treated with H₂O (3 mL), extracted with EA (2 mL×3), the combined extracts washed with brine (2 mL), dried over anhydrous Na₂SO₄, filtered, and concentrated to dryness under vacuum. The residue was purified by silica gel column chromatography, eluted with MeOH/DCM (0-10%) to afford (1R,3S)-3-(5-{2-[3-(benzyloxy)-5-cyano-2-formylphenoxy]acetamido}-2H-pyrazol-3-yl)cyclopentyl N-isopropylcarbamate (20.0 mg, 16.82%) as a white solid. MS (ESI) calcd. For C₂₉H₃₁N₅O₆, 545.02 m/z, found 546.20 [M+H]⁺.

### Step 7: Synthesis of (1R,3S)-3-(3-(2-(5-cyano-2-formyl-3-hydroxyphenoxy)acetamido)-1H-pyrazol-5-yl)cyclopentyl isopropylcarbamate

A solution of (1R,3S)-3-(5-{2-[3-(benzyloxy)-5-cyano-2-formylphenoxy]acetamido}-2H-pyrazol-3-yl)cyclopentyl N-isopropylcarbamate (20.0 mg, 0.037 mmol, 1 equiv) in TFA (2 mL) and methanesulfonic acid (0.4 mL) was stirred for 30 mins at rt. The resulting mixture was concentrated under reduced pressure and dissolved in DMF (1 mL). The crude product was then purified by Prep-HPLC: Column: Xselect CSH Prep C18 OBD Column, 30×150 mm, 5 um; Mobile Phase A: Water (0.1% FA), Mobile Phase B: ACN; Flow rate: 60 mL/min; Gradient: 26% B to 48% B in 10 min; Wave Length: 254 nm/220 nm. This resulted in (1R,3S)-3-{5-[2-(5-cyano-2-formyl-3-hydroxyphenoxy)acetamido]-2H-pyrazol-3yl}cyclopentyl N-isopropylcarbamate (1.5 mg, 8.56%) as a white solid. MS (ESI) calcd. For C₂₂H₂₅N₅O₆, 455.18 m/z, found 456.15 [M+H]⁺. ¹H NMR (300 MHz, DMSO-d₆) δ (ppm): 12.24 (s, 1H), 10.47 (s, 1H), 10.38 (s, 1H), 7.06 (s, 1H), 6.96 (d, J=7.9 Hz, 1H), 6.30 (s, 1H), 5.02 (s, 1H), 4.97 (s, 1H), 4.35 (t, J=5.1 Hz, 1H), 3.08 (s, 1H), 2.39 (s, 1H), 2.04-1.94 (m, 1H), 1.86 (s, 1H), 1.70 (d, J=9.2 Hz, 3H), 1.56 (s, 1H), 1.22 (s, 1H), 1.09 (s, 6H).

### Example 4.8: Synthesis of Compound 49: (1R,3S)-3-{5-[2-(2-formyl-3-methanesulfonamido phenoxy)acetamido]-2H-pyrazol-3-yl}cyclopentyl N-isopropyl carbamate

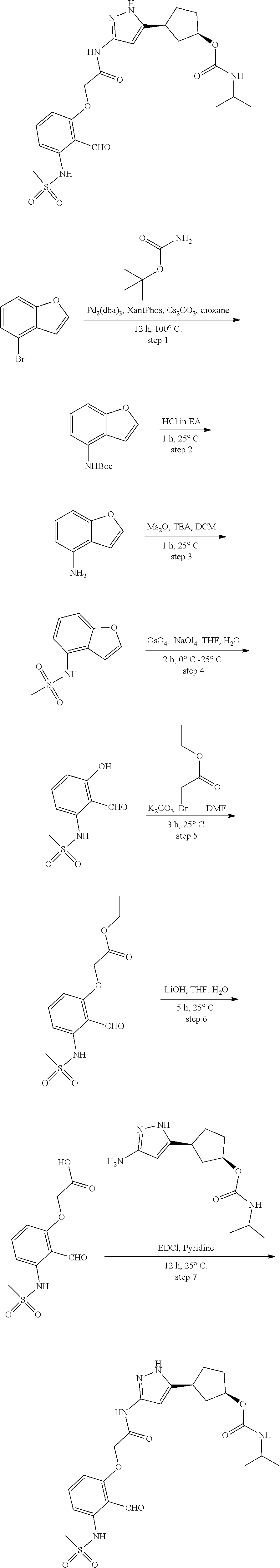

### Step 1: Synthesis of tert-butyl N-(1-benzofuran-4-yl)carbamate

A solution of 4-bromo-1-benzofuran (2 g, 10.151 mmol, 1 equiv), tert-butyl carbamate (11.89 g, 101.510 mmol, 10 equiv), Cs₂CO₃ (8.27 g, 25.377 mmol, 2.5 equiv), Pd₂(dba)₃ (929.5 mg, 1.015 mmol, 0.1 equiv), XantPhos (1.17 g, 2.030 mmol, 0.2 equiv) in dioxane (20 mL) was stirred for 12 h at 100° C. under nitrogen atmosphere. The mixture was diluted with EA (30 mL). The resulting mixture was washed with H₂O (30 mL×3), dried over anhydrous Na₂SO₄. The resulting mixture was filtered, a the filtrate concentrated under reduced pressure. The residue was purified by silica gel column chromatography, eluted with EA:PE (9:1) to afford tert-butyl N-(1-benzofuran-4-yl)carbamate (2.05 g, 84.07%) as a yellow oil. MS (ESI) calcd. for C₁₃H₁₅NO₃, 233.10 m/z, found 234.10 [M+H]⁺.

### Step 2: Synthesis of 1-benzofuran-4-amine

A solution of tert-butyl N-(1-benzofuran-4-yl)carbamate (2.05 g, 8.788 mmol, 1 equiv) in 1 M HCl in EA (15 mL) was stirred for 1 h at 25° C. The precipitated solids were collected by filtration and washed with EA (20 mL×3). This resulted in 1-benzofuran-4-amine (1.1 g, 90.34%) as a white solid. MS (ESI) calcd. for C₈H₇NO, 133.10 m/z, found 134.10 [M+H]⁺.

### Step 3: Synthesis of N-(1-benzofuran-4-yl)methanesulfonamide

A solution of 1-benzofuran-4-amine (1 g, 7.510 mmol, 1 equiv), methanesulfonic anhydride (999.2 mg, 6.008 mmol, 0.8 equiv) and TEA (1.52 g, 15.020 mmol, 2 equiv) in DCM (20 mL) was stirred for 1 h at 25° C. The mixture was diluted with DCM (20 mL). The resulting mixture was washed with H2O (30 mL×3), dried over anhydrous Na₂SO₄. The resulting mixture was concentrated under reduced pressure. The residue was purified by silica gel column chromatography, eluted with DCM:PE (1:9) to afford N-(1-benzofuran-4-yl)methanesulfonamide (900 mg, 54.23%) as a red oil. MS (ESI) calcd. for C₉H₉NO₃S, 211.10 m/z, found 212.10 [M+H]⁺.

### Step 4: Synthesis of Synthesis of 3-fluoro-2-hydroxy-5-(2-((2-methoxy-4-(4-(4-methylpiperazin-1-yl)piperidin-1-yl)phenyl)amino)-4-(phenylamino)pyrimidin-5-yl)benzaldehyde

A solution of N-(1-benzofuran-4-yl)methanesulfonamide (500 mg, 2.367 mmol, 1 equiv) in THF (4 mL) and H₂O (4 mL) was treated with OsO₄ (2.37 mL, 2.367 mmol, 1 equiv) for 30 min at 0° C. under nitrogen atmosphere followed by the addition of NaIO₄ (1.52 g, 7.101 mmol, 3 equiv) in portions at 25° C. The mixture was stirred for 2 h at 25° C. Desired product could be detected by LCMS. The mixture was diluted with EA (10 mL). The resulting mixture was washed with H₂O (10×3 mL), dried over anhydrous Na₂SO₄. The resulting solution was filtered and concentrated under reduced pressure. The crude product mixture was used in the next step directly without further purification. MS (ESI) calcd. for C₈H₉NO₄S, 215.10 m/z, found 216.10 [M+H]⁺.

### Step 5: Synthesis of ethyl 2-(2-formyl-3-methanesulfonamidophenoxy)acetate

A solution of N-(2-formyl-3-hydroxyphenyl)methanesulfonamide (200 mg, 0.929 mmol, 1 equiv) in DMF (5 mL) was treated with K2CO₃ (64.2 mg, 0.465 mmol, 0.5 equiv) for 30 mins at 25° C. followed by the addition of ethyl bromoacetate (77.6 mg, 0.465 mmol, 0.5 equiv) dropwise at 25° C. The mixture was stirred for 3 h at 25° C. The mixture was diluted with EA (10 mL). The resulting mixture was washed with H₂O (10 mL×3), dried over anhydrous Na₂SO₄. The resulting mixture was concentrated under vacuum. The residue was purified by silica gel column chromatography, eluted with MeOH:DCM (1:10) to afford ethyl 2-(2-formyl-3-methane sulfonamidophenoxy)acetate (140 mg, 40.05%) as a white solid. MS (ESI) calcd. for C₁₂H₁₅NO₆S, 301.10 m/z, found 302.10 [M+H]⁺.

### Step 6: Synthesis of 2-formyl-3-methanesulfonamidophenoxyacetic acid

A solution of ethyl 2-(2-formyl-3-methanesulfonamidophenoxy)acetate (90 mg, 0.299 mmol, 1 equiv) and LiOH (8.6 mg, 0.359 mmol, 1.2 equiv) in H₂O (0.4 mL) and THF (2 mL) was stirred for 5 h at 25° C. The resulting mixture was concentrated under reduced pressure. The mixture was neutralized to pH 7 with HCl (2 M). The mixture was diluted with EA (10 mL). The resulting mixture was washed with H₂O (10 mL×3), dried over anhydrous Na₂SO₄. After filtration, the filtrate was concentrated under reduced pressure resulting in 2-formyl-3-methanesulfonamidophenoxy acetic acid (80 mg, 83.41%) as a yellow solid. MS (ESI) calcd. for C₁₀H₁₁NO₆S, 273.20 m/z, found 274.20 [M+H]⁺.

### Step 7: Synthesis of (1R,3S)-3-{5-[2-(2-formyl-3-methanesulfonamidophenoxy)acetamido]-2H-pyrazol-3-yl}cyclopentyl N-isopropylcarbamate

A solution of 2-formyl-3-methanesulfonamidophenoxyacetic acid (73.1 mg, 0.267 mmol, 1.5 equiv), (1R,3S)-3-(5-amino-2H-pyrazol-3-yl)cyclopentyl N-isopropylcarbamate (45 mg, 0.178 mmol, 1.00 equiv) and EDCI (51.3 mg, 0.267 mmol, 1.5 equiv) in pyridine (2 mL) was stirred for 12 h at 25° C. The mixture was diluted with EA (10 mL). The resulting mixture was washed with H₂O (10 mL×3), dried over anhydrous Na₂SO₄. The resulting mixture was concentrated under reduced pressure. The crude product was purified by Column: Xselect CSH Prep C18 OBD Column, 30×150 mm, 5 um; Mobile Phase A: Water (0.1% FA), Mobile Phase B: ACN; Flow rate: 60 mL/min; Gradient: 23% B to 45% B in 10 min; Wave Length: 254/220 nm nm to afford (1R,3S)-3-{5-[2-(2-formyl-3-methanesulfonamidophenoxy)acetamido]-2H-pyrazol-3-yl}cyclopentyl N-isopropylcarbamate (3.7 mg, 4.07%) as a white solid. MS (ESI) calcd. for C₂₂H₂₉N₅₀₇S, 507.25 m/z, found 508.25 [M+H]⁺. ¹H NMR (400 MHz, DMSO-d₆) δ (ppm): 12.15 (s, 1H), 10.85 (s, 1H), 10.57 (s, 1H), 10.48 (s, 1H), 7.61 (s, 1H), 7.14 (d, J=8.4 Hz, 1H), 6.93 (d, J=7.9 Hz, 1H), 6.76 (s, 1H), 6.32 (s, 1H), 4.99 (s, 1H), 4.86 (s, 2H), 3.51 (s, 2H), 3.15 (s, 2H), 2.95 (s, 1H), 2.01 (d, J=9.4 Hz, 1H), 1.88 (s, 1H), 1.70 (s, 3H), 1.25 (s, 1H), 1.03 (d, J=6.4 Hz, 6H).

### Example 4.9: Synthesis of Compound 51: (1R,3S)-3-(3-(2-(3-ethynyl-2-formylphenoxy)acetamido)-1H-pyrazol-5-yl)cyclopentyl isopropylcarbamate

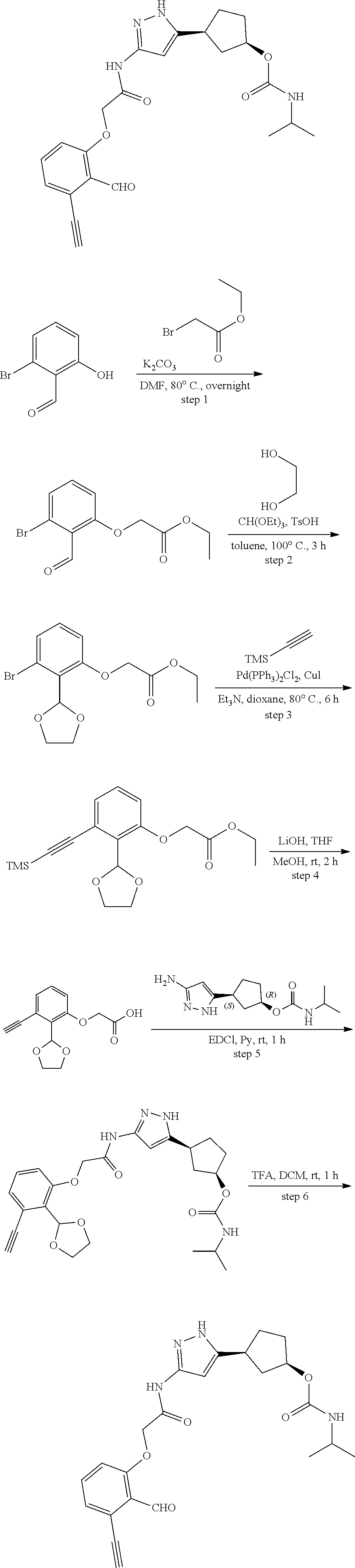

### Step 1: Synthesis of ethyl 2-(3-bromo-2-formylphenoxy)acetate

2-bromo-6-hydroxybenzaldehyde (3 g, 14.924 mmol), a stir bar and DMF (40 mL) were added to a 250 mL round-bottom flask and stirred until homogeneous, and then treated with K₂CO₃ (2.1 g, 15.085 mmol), ethyl 2-bromoacetate (5 g, 29.940 mmol). The resulting mixture was stirred overnight at 80° C. under N₂ atmosphere, then cooled to r.t and diluted with water. The resulting mixture was extracted with EA (2×100 mL). The combined organic layers were washed with brine, dried over anhydrous sodium sulfate, filtered, and concentrated under vacuum. The residue was purified by silica gel chromatography (0-40% EA/PE) to afford ethyl 2-(3-bromo-2-formylphenoxy)acetate as a yellow solid (4.2 g, 98.02% yield). MS (ESI) mass calcd. for C₁₁H₁₁BrO₄, 285.98 m/z, found, 286.90 [M+H]⁺.

### Step 2: Synthesis of ethyl 2-(3-bromo-2-(1,3-dioxolan-2-yl)phenoxy)acetate

Ethyl 2-(3-bromo-2-formylphenoxy)acetate (4.2 g, 14.872 mmol), a stir bar and toluene (40 mL) were added to a 250 mL round-bottom flask and stirred until homogeneous, and then treated with ethane-1,2-diol (4.7 g, 75.698 mmol), triethoxymethane (6.70 g, 45.211 mmol), TsOH (0.28 g, 1.636 mmol). The resulting mixture was stirred for 3 h at 100° C., then cooled to r.t and concentrated under vacuum. The residue was purified by silica gel chromatography (0-35% EA/PE) to afford ethyl 2-[3-bromo-2-(1,3-dioxolan-2-yl)phenoxy]acetate as a yellow solid (4.6 g, 93.40% yield). MS (ESI) mass calcd. for C₁₃H₁₅BrO₅, 330.01 m/z, found, 331.00 [M+H]⁺.

### Step 3: Synthesis of ethyl 2-[2-(1,3-dioxolan-2-yl)-3-[2-(trimethylsilyl) ethynyl]phenoxy]acetate

Ethyl 2-[3-bromo-2-(1,3-dioxolan-2-yl)phenoxy]acetate (2 g, 6.039 mmol), trimethylsilylacetylene (2.4 g, 24.458 mmol), Et₃N (2.47 g, 24.398 mmol), a stir bar and dioxane (30 mL) were added to a 100 mL round-bottom flask and stirred until homogeneous, and then treated with Pd(PPh₃)₂Cl₂ (0.85 g, 1.208 mmol), CuI (0.26 g, 1.389 mmol). The resulting mixture was stirred for 6 h at 80° C., then cooled to r.t and diluted with water. The resulting mixture was extracted with DCM (100 mL) twice. The combined organic layers were washed with brine, dried over anhydrous sodium sulfate, filtered, and concentrated under vacuum. The residue was purified by silica gel chromatography (0-30% EA/PE) to afford ethyl 2-[2-(1,3-dioxolan-2-yl)-3-[2-(trimethylsilyl)ethynyl]phenoxy]acetate as a yellow solid (1.9 g, 90.28% yield). MS (ESI) mass calcd. for C₁₈H₂₄O₅Si, 348.13 m/z, found, 349.10 [M+H]⁺.

### Step 4: Synthesis of 2-(2-(1,3-dioxolan-2-yl)-3-ethynylphenoxy)acetic acid

Ethyl 2-[2-(1,3-dioxolan-2-yl)-3-[2-(trimethylsilyl)ethynyl]phenoxy]acetate (600 mg, 1.722 mmol), a stir bar, THF (5.1 mL) and MeOH (1.7 mL) were added to a 100 mL round-bottom flask and stirred until homogeneous, and then treated with LiOH (1.7 mL, 5.100 mmol, 3 mol/L). The reaction mixture was stirred for 1 h at r.t, then diluted with water (1.7 mL). The pH of the resulting mixture was adjusted to 4-5 with 2 M HCl. The resulting mixture was extracted with EA (50 mL) twice. The combined extracts were washed with brine, dried over anhydrous sodium sulfate, filtered, and concentrated under vacuum to afford 2-(1,3-dioxolan-2-yl)-3-ethynylphenoxyacetic acid as a yellow solid (150 mg, crude). MS (ESI) mass calcd. for C₁₃H₁₂O₅, 248.07 m/z, found, 249.10 [M+H]⁺.

### Step 5: Synthesis of (1R,3S)-3-(3-(2-(2-(1,3-dioxolan-2-yl)-3-ethynylphenoxy)acetamido)-1H-pyrazol-5-yl)cyclopentyl isopropylcarbamate

2-(1,3-dioxolan-2-yl)-3-ethynylphenoxyacetic acid (120 mg, 0.483 mmol), (1R,3S)-3-(5-amino-2H-pyrazol-3-yl)cyclopentyl N-isopropylcarbamate (100 mg, 0.396 mmol), a stir bar, pyridine (2 mL) were added to a 8 mL vial and stirred until homogeneous, then treated with EDCI (120 mg, 0.626 mmol). The reaction mixture was stirred for 1 h at r.t, then diluted with water. The resulting mixture was extracted with EA (2×30 mL). The combined organic layers were washed with brine, dried over anhydrous sodium sulfate, filtered, and concentrated under vacuum. The residue was purified by reverse-phase chromatography (5-60% ACN/H₂O with 10 mM NH₄HCO₃) to afford (1R,3S)-3-(5-(2-[2-(1,3-dioxolan-2-yl)-3-ethynylphenoxy]acetamido-2H-pyrazol-3-yl)cyclopentyl N-isopropylcarbamate as a yellow solid (46 mg, 24.05%). MS (ESI) mass calcd. for C₂₅H₃₀N₄O₆, 482.21 m/z, found, 483.25 [M+H]⁺.

### Step 6: Synthesis of (1R,3S)-3-(3-(2-(3-ethynyl-2-formylphenoxy)acetamido)-1H-pyrazol-5-yl)cyclopentyl isopropylcarbamate

(1R,3S)-3-(5-(2-[2-(1,3-dioxolan-2-yl)-3-ethynylphenoxy]acetamido-2H-pyrazol-3-yl)cyclopentyl N-isopropylcarbamate (46 mg, 0.095 mmol), a stir bar and DCM (2.5 mL) were added to a 8 mL vial and stirred until homogeneous, and then treated with TFA (0.5 mL). The resulting mixture was stirred for 1 h at room temperature, then concentrated under vacuum and purified by Prep-HPLC with (Column: Xselect CSH OBD Column, 30*150 mm, 5 um; Mobile Phase A: Water (0.05% TFA), Mobile Phase B: ACN; Flow rate: 60 mL/min; Gradient: 29% B to 52% B in 10 min; Wave Length: 254 nm/220 nm nm; RT1 (min): 8.5) to afford (1R,3S)-3-(5-[2-(3-ethynyl-2-formylphenoxy)acetamido]-2H-pyrazol-3-ylcyclopentyl N-isopropylcarbamate as a white solid (24.1 mg, 57.49% yield, MS (ESI) mass calcd. for C₂₃H₂₆N₄O₅, 438.19 m/z, found, 439.15 [M+H]⁺). ¹H NMR (400 MHz, DMSO-d₆) δ 10.52 (s, 1H), 10.39 (s, 1H), 7.61 (t, J=8.1 Hz, 1H), 7.23 (dd, J=19.0, 8.1 Hz, 2H), 6.98-6.89 (m, 1H), 6.32 (s, 1H), 4.99 (s, 1H), 4.85 (s, 2H), 4.55 (s, 1H), 3.63-3.51 (m, 1H), 3.10-3.01 (m, 1H), 2.48-2.42 (m, 1H), 2.05-1.82 (m, 2H), 1.77-1.54 (m, 3H), 1.02 (d, J=6.5 Hz, 6H).

### Example 4.10: Synthesis of Compound 56: (1R,3S)-3-(3-(2-(2-formyl-3-hydroxy-4-methylphenoxy)acetamido)-1H-pyrazol-5-yl)cyclopentyl isopropylcarbamate

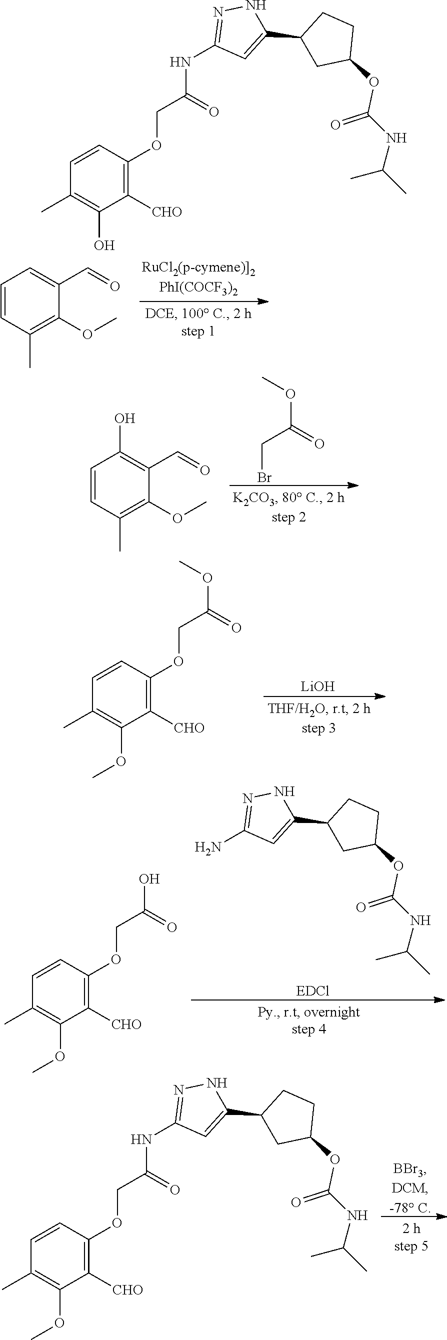

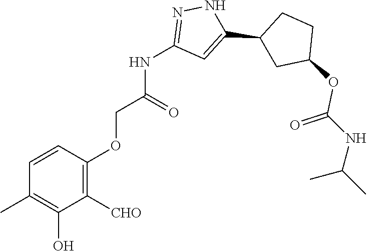 [SMILES: Cc1ccc(OCC(=O)Nc2cc([C@H]3CC[C@@H](OC(=O)NC(C)C)C3)[nH]n2)c(C=O)c1O]

### Step 1: Synthesis of 6-hydroxy-2-methoxy-3-methylbenzaldehyde

A solution of 2-methoxy-3-methylbenzaldehyde (2.0 g, 13.318 mmol, 1.00 equiv) in DCE (30 mL) was treated with phenyl 2,2,2-trifluoroacetyl trifluoroacetyl iodide (10.6 g, 26.636 mmol, 2 equiv) and [RuCl₂(p-cymene)]2 (815.6 mg, 1.332 mmol, 0.10 equiv) and stirred for 2 h at 100° C. under nitrogen atmosphere. The reaction mixture was then treated with H₂O (60 mL) and extracted with DCM (30 mL×3), and the combined extracts washed with brine (30 mL), dried over anhydrous Na₂SO₄, filtered, and concentrated to dryness under vacuum. The residue was purified by silica gel column chromatography, eluted with MeOH/DCM (0-8%) to afford 6-hydroxy-2-methoxy-3-methylbenzaldehyde (244.0 mg, 22.05%) as a yellow oil. MS (ESI) calcd. for C₉H₁₀O₃, 166.06 m/z, found 167.15 [M+H]⁺.

### Step 2: Synthesis of methyl 2-(2-((l1-oxidaneyl)-l3-methyl)-3-methoxy-4-methylphenoxy)acetate

A solution of 6-hydroxy-2-methoxy-3-methylbenzaldehyde (300.0 mg, 1.805 mmol, 1 equiv) in DMF (10 mL) was treated with methyl 2-bromoacetate (331.4 mg, 2.166 mmol, 1.2 equiv) and K₂CO₃ (374.6 mg, 2.708 mmol, 1.5 equiv) and stirred for 2 h at 80° C. The reaction mixture was then treated with H₂O (20 mL) and extracted with EA (10 mL×3), and the combined extracts washed with brine (10 mL), dried over anhydrous Na₂SO₄, filtered, and concentrated to dryness under vacuum. The residue was purified by silica gel column chromatography, eluted with PE/EA (0-30%) to afford methyl 2-(2-formyl-3-methoxy-4-methylphenoxy)acetate (350.0 mg, 81.38%) as a yellow oil. MS (ESI) calcd. For C₁₂H₁₄O₅, 238.08 m/z, found 239.15 [M+H]⁺.

### Step 3: Synthesis of 2-(2-formyl-3-methoxy-4-methylphenoxy)acetic acid

A solution of methyl 2-(2-formyl-3-methoxy-4-methylphenoxy)acetate (300.0 mg, 1.259 mmol, 1 equiv) in THF (6 mL) and H₂O (2 mL) was treated with LiOH (75.4 mg, 3.147 mmol, 2.5 equiv) and stirred for 2 h at rt. The mixture was acidified to pH 6 with HCl (2 mol/L) and mixture was extracted with EA (10 mL×3), and the combined extracts washed with brine (10 mL), dried over anhydrous Na₂SO₄, filtered, and concentrated to dryness under vacuum. The resulting mixture was concentrated under reduced pressure to afford 2-formyl-3-methoxy-4-methylphenoxyacetic acid (150.0 mg, 53.13%) as a yellow solid. MS (ESI) calcd. For C₁₁H₁₂O₅, 224.14 m/z, found 225.15 [M+H]⁺.

### Step 4: Synthesis of (1R,3S)-3-(3-(2-(2-formyl-3-methoxy-4-methylphenoxy)acetamido)-1H-pyrazol-5-yl)cyclopentyl isopropylcarbamate

2-Formyl-3-methoxy-4-methylphenoxyacetic acid (100.0 mg, 0.446 mmol, 1 equiv), EDCI (128.2 mg, 0.669 mmol, 1.5 equiv) and (1R,3S)-3-(3-amino-1H-pyrazol-5-yl)cyclopentyl isopropylcarbamate (105.4 mg, 0.467 mmol, 1 equiv) were dissolved in pyridine (1 mL) and stirred overnight at rt. The reaction mixture was then treated with H₂O (3 mL) and extracted with EA (2 mL×3), and the combined extracts washed with brine (2 mL), dried over anhydrous Na₂SO₄, filtered, and concentrated to dryness under vacuum. The residue was purified by silica gel column chromatography, eluted with PE/EA (0-30%) to afford (1R,3S)-3-{5-[2-(2-formyl-3-methoxy-4-methylphenoxy)acetamido]-2H-pyrazol-3-yl}cyclopentyl N-isopropylcarbamate (150.0 mg, 73.35%) as a yellow solid. MS (ESI) calcd. For C₂₃H₃₀N₄O₆, 458.21 m/z, found 459.15 [M+H]⁺.

### Step 5: Synthesis of (1R,3S)-3-(3-(2-(2-formyl-3-hydroxy-4-methylphenoxy)acetamido)-1H-pyrazol-5-yl)cyclopentyl isopropylcarbamate

A solution of (1R,3S)-3-{5-[2-(2-formyl-3-methoxy-4-methylphenoxy)acetamido]-2H-pyrazol-3-yl}cyclopentyl N-isopropylcarbamate (100.0 mg, 0.218 mmol, 1 equiv) in DCM (10 mL) was treated with boron tribromide (2.2 mL, 2.180 mmol, 10 equiv, 1M in DCM) and stirred for 2 h at −78° C. under nitrogen atmosphere. The reaction mixture was then treated with NaHCO₃ (20 mL) and extracted with DCM (10 mL×2), and the combined extracts washed with brine (10 mL), dried over anhydrous Na₂SO₄, filtered, and concentrated to dryness under vacuum. The crude product was then purified by Prep-HPLC with the following conditions: Column: Xselect CSH OBD Column, 30×150 mm, 5 um; Mobile Phase A: Water (0.1% FA), Mobile Phase B: ACN; Flow rate: 60 mL/min; Gradient: 32% B to 54% B in 10 min; Wave Length: 254/220 nm; RT1 (min): 9. This resulted in (1R,3S)-3-{5-[2-(2-formyl-3-hydroxy-4-methylphenoxy)acetamido]-2H-pyrazol-3-yl}cyclopentyl N-isopropylcarbamate (7.4 mg, 7.14%) as a white solid. MS (ESI) calcd. For C₂₂H₂₈N₄O₆, 444.50 m/z, found 445.15 [M+H]⁺. ¹H NMR (300 MHz, DMSO-d₆) δ (ppm): 12.13 (s, 2H), 10.51 (s, 1H), 10.38 (s, 1H), 7.40 (d, J=8.6 Hz, 1H), 6.92 (d, J=7.7 Hz, 1H), 6.44 (d, J=8.4 Hz, 1H), 6.30 (s, 1H), 4.98 (s, 1H), 4.79 (s, 2H), 3.62-3.50 (m, 1H), 3.10-2.98 (m, 1H), 2.43 (s, 1H), 2.08 (s, 3H), 2.04-1.84 (m, 1H), 1.87-1.82 (m, 1H), 1.75-1.64 (m, 2H), 1.63-1.54 (m, 1H), 1.02 (d, J=6.6 Hz, 6H).

### Example 4.11: Synthesis of Compound 57: (1R,3S)-3-[5-(3-chloro-5-formyl-4-hydroxybenzamido)-2H-pyrazol-3-yl]cyclopentyl N-isopropylcarbamate

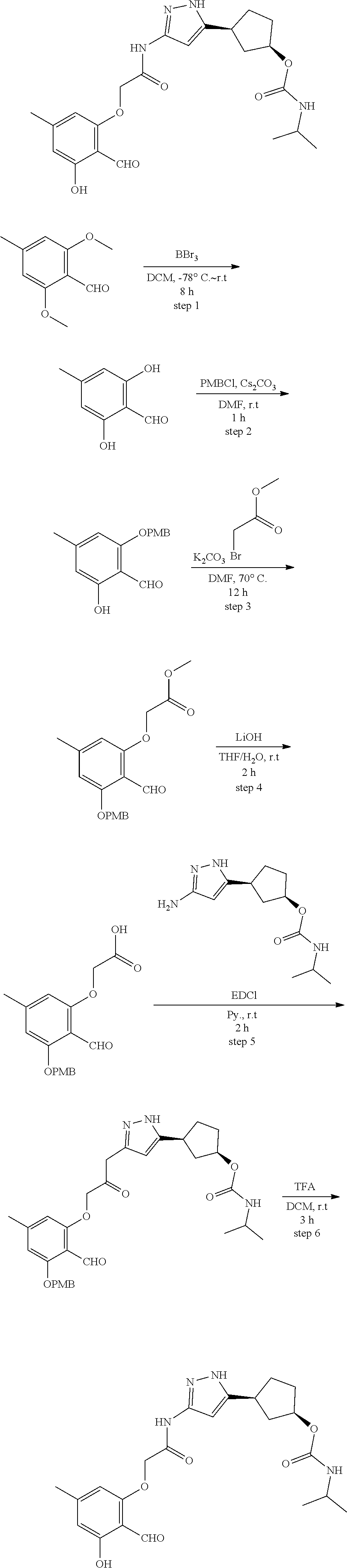

### Step 1: Synthesis of 2,6-dihydroxy-4-methylbenzaldehyde

A solution of 2,6-dimethoxy-4-methylbenzaldehyde (2.0 g, 11.099 mmol, 1.0 equiv) in DCM (20 mL) was added boron tribromide (66.59 mL, 66.594 mmol, 6.0 equiv) dropwise at −78° C. under nitrogen atmosphere. The resulting mixture was stirred for 2 hours at −78° C., then stirred for 2 hours at 0° C. and stirred for 4 hours at rt under N₂ atmosphere. The reaction was quenched with saturated sodium bicarbonate solution at 0° C. then diluted with DCM (50 mL×3). The combined organic layers were washed with saturated sodium chloride solution (50 mL), dried over anhydrous Na₂SO₄, concentrated under reduced pressure. The residue was purified by silica gel column chromatography, eluted with PE/EA (10%) to afford 2,6-dihydroxy-4-methylbenzaldehyde (657.0 mg, 37.62%) as a white solid. MS (ESI) calcd. for C₈H₈O₃, 152.05 m/z, found 151.00 [M−H]⁺.

### Step 2: Synthesis of 2-hydroxy-6-((4-methoxybenzyl)oxy)-4-methylbenzaldehyde

A solution of 2,6-dihydroxy-4-methylbenzaldehyde (650.0 mg, 4.272 mmol, 1.0 equiv) and Cs₂CO₃ (835.2 mg, 2.563 mmol, 0.6 equiv) in DMF (10 mL) was stirred for 15 mins at rt. To the stirred solution was added PMBCl (0.66 mL, 4.699 mmol, 1.1 equiv) dropwise at rt. and left to stir at rt for 1 hour. The resulting mixture was diluted with EA (50 mL) and washed with H₂O (20 mL×3). The organic extract was washed with saturated sodium chloride solution (30 mL), dried over anhydrous Na₂SO₄, concentrated under reduced pressure. The residue was purified by silica gel column chromatography, eluted with PE/EA (10%) to afford 2-hydroxy-6-[(4-methoxyphenyl)methoxy]-4-methylbenzaldehyde (560.0 mg, 48.14%) as a white solid. MS (ESI) calcd. for C₁₆H₁₆O₄, 272.10 m/z, found 271.10 [M+H]⁺.

### Step 3: Synthesis of methyl 2-(2-formyl-3-((4-methoxybenzyl)oxy)-5-methylphenoxy)acetate

A solution of 2-hydroxy-6-[(4-methoxyphenyl)methoxy]-4-methylbenzaldehyde (560.0 mg, 2.057 mmol, 1.0 equiv) and methyl 2-bromoacetate (471.9 mg, 3.085 mmol, 1.5 equiv) in DMF (5 mL) was stirred at 0° C. for 20 minutes followed by the addition of methyl 2-bromoacetate (471.9 mg, 3.085 mmol, 1.5 equiv) dropwise at 0° C. The resulting mixture was stirred overnight at rt. The reaction mixture was diluted with EA (30 mL) and was washed with H₂O (20 mL×3). The combined organic layers were washed with saturated sodium chloride solution (30 mL), dried over anhydrous Na₂SO₄, filtered, and the filtrate was concentrated under reduced pressure. The residue was purified by silica gel column chromatography, eluted with MeOH/DCM (3%) to afford methyl 2-{2-formyl-3-[(4-methoxyphenyl)methoxy]-5-methylphenoxy}acetate (591.0 mg, 73.44%) as a light yellow solid. MS (ESI) calcd. for C₁₉H₂₀O₆, 344.13 m/z, found 343.00 [M−H]⁺

### Step 4: Synthesis of 2-(2-formyl-3-((4-methoxybenzyl)oxy)-5-methylphenoxy)acetic acid

A solution of methyl 2-{2-formyl-3-[(4-methoxyphenyl)methoxy]-5-methylphenoxy}acetate (300.0 mg, 0.871 mmol, 1.0 equiv) and LiOH H₂O (65.8 mg, 1.568 mmol, 1.8 equiv) in THF (10 mL) and H₂O (2 mL) was stirred for 2 hours at rt. The resulting mixture was concentrated under reduced pressure. The residue was neutralized to pH 7 with HCl (2M). The aqueous layer was extracted with EA (30 mL×3). The resulting mixture was concentrated under reduced pressure to afford 2-formyl-3-[(4-methoxyphenyl)methoxy]-5-methylphenoxyacetic acid (206.0 mg, 67.29%) as a white solid. MS (ESI) calcd. for C₁₈H₁₈O₆, 330.11 m/z, found 329.00 [M−H]⁺.

### Step 5: Synthesis of (1R,3S)-3-(3-(2-(2-formyl-3-((4-methoxybenzyl)oxy)-5-methylphenoxy)acetamido)-1H-pyrazol-5-yl)cyclopentyl isopropylcarbamate

A solution of 2-formyl-3-[(4-methoxyphenyl)methoxy]-5-methylphenoxyacetic acid (157.1 mg, 0.475 mmol, 1.2 equiv) and (1R,3S)-3-(5-amino-2H-pyrazol-3-yl)cyclopentyl N-isopropylcarbamate (100.0 mg, 0.396 mmol, 1.00 equiv) and EDCI (114.0 mg, 0.594 mmol, 1.5 equiv) in pyridine (3 mL) was stirred for 2 hours at rt. The resulting mixture was concentrated under reduced pressure and purified by Prep-TLC (MeOH/DCM=1:10) to afford (1R,3S)-3-[5-(2-{2-formyl-3-[(4-methoxyphenyl)methoxy]-5-methylphenoxy}acetamido)-2H-pyrazol-3-yl]cyclopentyl N-isopropylcarbamate (133.0 mg, 19.38%) as alight yellow solid. MS (ESI) calcd. for C₃₀H₃₆N₄O₇, 564.26 m/z, found 565.30 [M+H]⁺.

### Step 6: Synthesis of (1R,3S)-3-(3-(2-(2-formyl-3-hydroxy-5-methylphenoxy)acetamido)-1H-pyrazol-5-yl)cyclopentyl isopropylcarbamate

A solution of (1R,3S)-3-[5-(2-{2-formyl-3-[(4-methoxyphenyl)methoxy]-5-methylphenoxy}acetamido)-2H-pyrazol-3-yl]cyclopentyl N-isopropylcarbamate (120.0 mg, 0.213 mmol, 1 equiv) in DCM (1.5 mL) was treated with trifluoroacetic acid (0.5 mL) at rt. The resulting mixture was stirred for 3 hours at rt. The crude product was purified by Prep-HPLC with the following conditions (Column: XBridge Shield RP18 OBD Column, 30×150 mm, 5 um; Mobile Phase A: Water (0.1% FA), Mobile Phase B: ACN; Flow rate: 60 mL/min; Gradient: 31% B to 53% B in 10 min; Wave Length: 254/220 nm; RT1 (min): 8) to afford (1R,3S)-3-{5-[2-(2-formyl-3-hydroxy-5-methylphenoxy)acetamido]-2H-pyrazol-3-yl}cyclopentyl N-isopropylcarbamate (1.0 mg, 1.04%) as a white solid. MS (ESI) calcd. for C₂₂H₂₈N₄O₆, 444.20 m/z, found 445.20 [M+H]⁺. ¹H NMR (400 MHz, DMSO-d₆) δ 12.16 (s, 1H), 11.74 (s, 1H), 10.51 (s, 1H), 10.30 (s, 1H), 6.94 (d, J=7.9 Hz, 1H), 6.39 (d, J=7.8 Hz, 2H), 6.32 (s, 1H), 4.99 (s, 1H), 4.80 (s, 2H), 4.36 (t, J=5.1 Hz, 1H), 2.27 (s, 3H), 2.01-1.99 (m, 1H), 1.93-1.87 (m, 1H), 1.70-1.68 (m, 2H), 1.61-1.57 (m, 1H), 1.24 (s, 1H), 1.04-1.01 (m, 6H).

### Example 4.12: Synthesis of Compound 64: (1R,3S)-3-{5-[2-(2-formyl-3-hydroxy-5-methoxy phenoxy)acetamido]-2H-pyrazol-3-yl}cyclopentyl N-isopropylcarbamate

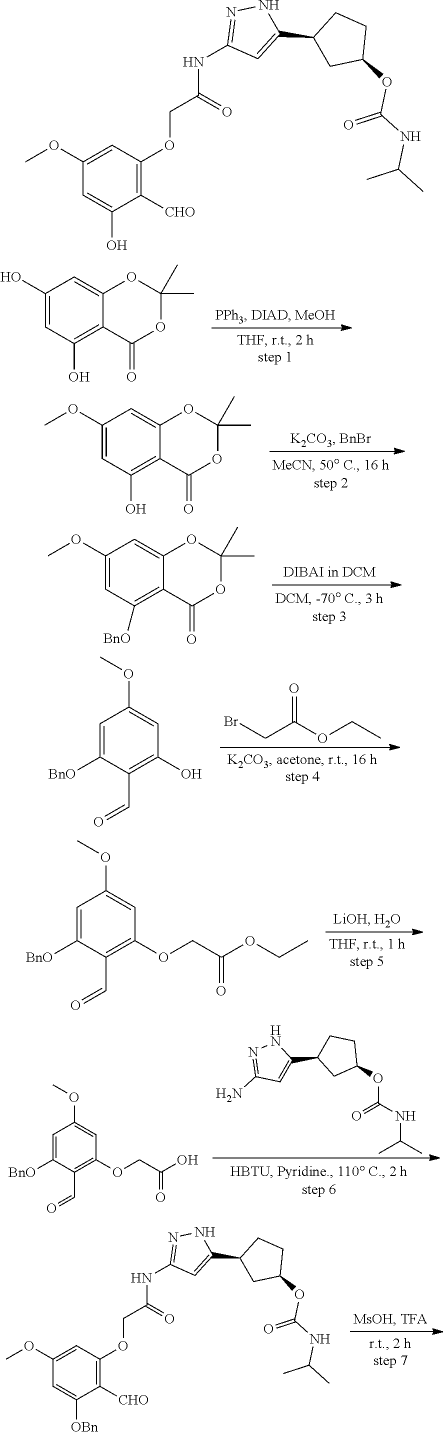

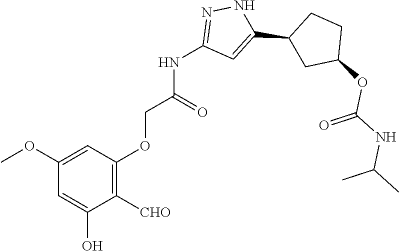 [SMILES: COc1cc(O)c(C=O)c(OCC(=O)Nc2cc([C@H]3CC[C@@H](OC(=O)NC(C)C)C3)[nH]n2)c1]

### Step 1: Synthesis of 5-hydroxy-7-methoxy-2,2-dimethyl-1,3-benzodioxin-4-one

5,7-dihydroxy-2,2-dimethyl-1,3-benzodioxin-4-one (1 g, 4.758 mmol), triphenylphosphine (1.6 g, 6.185 mmol), methanol (0.2 g, 6.185 mmol) and tetrahydrofuran (15 mL) were added to a 100 mL round-bottom flask and stirred until homogeneous, then treated with DIAD (1.3 g, 6.185 mmol) at 0° C. The resulting mixture was stirred under N₂ atmosphere. The resulting mixture was stirred at rt. for 2 h and quenched with H₂O extracted with EA (100 mL×3). The combined organic layers were washed with brine, dried over anhydrous sodium sulfate, filtered, and concentrated under vacuum. The residue was purified by silica gel chromatography (0-40% PE/EA) to afford 5-hydroxy-7-methoxy-2,2-dimethyl-1,3-benzodioxin-4-one as a white solid (1 g, 93.74%). MS (ESI) mass calcd. for C₁₁H₁₂O₅, 224.06 m/z, found 223.10 [M−H]⁻.

### Step 2: Synthesis of 5-(benzyloxy)-7-methoxy-2,2-dimethyl-1,3-benzodioxin-4-one

5-hydroxy-7-methoxy-2,2-dimethyl-1,3-benzodioxin-4-one (1 g, 4.460 mmol), a stir bar, K₂CO₃ (1.2 g, 8.920 mmol) and ACN (16 mL) were added to a 100 mL round-bottom flask and stirred until homogeneous, then treated with benzyl bromide (1.1 g, 6.690 mmol). The reaction mixture was stirred at 50° C. overnight and quenched with NH₄Cl, extracted with EA (150 mL×3). The combined organic layers were washed with brine, dried over anhydrous sodium sulfate, filtered, and concentrated under vacuum. The residue was purified by silica gel chromatography (0-40% PE/EA) to afford 5-(benzyloxy)-7-methoxy-2,2-dimethyl-1,3-benzodioxin-4-one as a yellow oil (1.3 g, 92.73%). MS (ESI) mass calcd. for C₁₈H₁₈O₅, 314.11 m/z, found 315.10 [M+H]⁺.

### Step 3: Synthesis of 2-(benzyloxy)-6-hydroxy-4-methoxybenzaldehyde

5-(benzyloxy)-7-methoxy-2,2-dimethyl-1,3-benzodioxin-4-one (1.3 g, 4.136 mmol), a stir bar, and DCM (20 mL) were added to a 100 mL round-bottom flask and stirred until homogeneous. The resulting mixture was stirred under N₂ atmosphere. The resulting mixture was stirred at −70° C. for 0.5 h, followed by the dropwise addition of DIBAl (8.27 mL, 12.408 mmol, 1.5 M in toluene) −70° C. The resulting mixture was stirred at −70° C. for 3 h and quenched with MeOH (1 mL). The mixture was acidified with HCl aqueous (1 M) until pH=1. Mixture was left to stir at room temperature for 3 h. The reaction mixture was extracted with DCM (3×90 mL), the combined organic phase was washed with brine, and dried with Na₂SO₄, filtered, and evaporated at reduced pressure. The residue was added to a combined solution of THF (5 mL) and HCl aqueous (2 M, 1 mL), and stirred at room temperature for 2 h. Then the mixture was diluted with H₂O, extracted with EA (3×90 mL), the combined organic phase was washed with brine, and dried with Na₂SO₄, filtered, and evaporated at reduced pressure. The residue was purified by silica gel chromatography (0-30% PE/EA) to afford 2-(benzyloxy)-6-hydroxy-4-methoxybenzaldehyde as a white solid (0.5 g, 46.81%). MS (ESI) mass calcd. for C₁₅H₁₄O₄, 258.08 m/z, found 259.00 [M+H]⁺.

### Step 4: Synthesis of Ethyl 2-[3-(benzyloxy)-2-formyl-5-methoxyphenoxy]acetate

2-(benzyloxy)-6-hydroxy-4-methoxybenzaldehyde (400 mg, 1.549 mmol), a stir bar, K₂CO₃ (431 mg, 3.098 mmol) and acetone (10 mL) were added to a 100 mL round-bottom flask and stirred until homogeneous, then treated with ethyl bromoacetate (517 mg, 3.098 mmol) under N₂ atmosphere. The reaction mixture was stirred at rt. overnight, then diluted with water. The resulting mixture was extracted with EA (100 mL×3). The combined organic layers were washed with H₂O (50 mL×3), dried over anhydrous Na₂SO₄. After filtration, the filtrate was concentrated under reduced pressure. The residue was purified by silica gel column with PE/EA (0-60%) to afford ethyl 2-[3-(benzyloxy)-2-formyl-5-methoxyphenoxy]acetate (250 mg, 46.88%) as a white solid. MS (ESI) mass calcd. for C₁₉H₂₀O₆, 344.12 m/z, found 345.05 [M+H]⁺.

### Step 5: Synthesis of 3-(benzyloxy)-2-formyl-5-methoxyphenoxyacetic acid

Ethyl 2-[3-(benzyloxy)-2-formyl-5-methoxyphenoxy]acetate (250 mg, 0.726 mmol), LiOH (24 mg, 1.016 mmol), tetrahydrofuran (5 mL) and, water (1 mL) were added to a 100 mL round-bottom flask and stirred until homogeneous. The resulting mixture was stirred at rt. for 1 h. The reaction mixture was acidified to pH=5 with HCl (2 M). The resulting mixture was extracted with EA (3×60 mL). The combined organic layers were washed with brine, dried over anhydrous sodium sulfate, filtered, and concentrated under vacuum to afford 3-(benzyloxy)-2-formyl-5-methoxyphenoxyacetic acid as a white solid (230 g, 95.80%). MS (ESI) calcd. for C₁₇H₁₆O₆, 316.09 m/z, found 317.10 [M+H]⁺.

### Step 6: Synthesis of (1R,3S)-3-(5-{2-[3-(benzyloxy)-2-formyl-5-methoxyphenoxy]acetamido}-2H-pyrazol-3-yl)cyclopentyl N-isopropylcarbamate

3-(benzyloxy)-2-formyl-5-methoxyphenoxyacetic acid (114 mg, 0.360 mmol), (1R,3S)-3-(5-amino-2H-pyrazol-3-yl)cyclopentyl N-isopropylcarbamate (70 mg, 0.277 mmol) and pyridine (5 mL) were added to a 40 mL vail and stirred until homogeneous, then treated with HBTU (126 mg, 0.332 mmol). The reaction mixture was stirred at 110° C. for 2 h. The reaction was quenched with H₂O and extracted with EA. The combined organic layers were washed with brine, dried over anhydrous sodium sulfate, filtered, and concentrated under vacuum. The residue was purified by reverse-phase chromatography (5-60%, ACN/H₂O with 5 mM NH₄HCO₃) to afford (1R,3S)-3-(5-{2-[3-(benzyloxy)-2-formyl-5-methoxyphenoxy]acetamido}-2H-pyrazol-3-yl)cyclopentyl N-isopropylcarbamate (40 mg, 26.19%) as a yellow solid. MS (ESI) mass calcd. for C₂₉H₃₄N₄O₇, 550.24 m/z, found 551.10 [M+H]⁺.

### Step 7: Synthesis of (1R,3S)-3-{5-[2-(2-formyl-3-hydroxy-5-methoxyphenoxy)acetamido]-2H-pyrazol-3-yl}cyclopentyl N-isopropylcarbamate

(1R,3S)-3-(5-{2-[3-(benzyloxy)-2-formyl-5-methoxyphenoxy]acetamido}-2H-pyrazol-3-yl) cyclopentyl N-isopropylcarbamate (40 mg, 0.109 mmol), methanesulfonic acid (3.6 mL) and TFA (1.2 mL) were added to a 20 mL vial and stirred until homogeneous. The reaction mixture was stirred at rt. For 2 h and concentrated under vacuum. The residue was purified by Prep-HPLC (Column: Xselect CSH Prep C18 OBD Column, 30×150 mm, 5 um; Mobile Phase A: Water (0.05% TFA), Mobile Phase B: ACN; Flow rate: 60 mL/min; Gradient: 28% B to 50% B in 10 min; Wave Length: 254/220 nm) to afford (1R,3S)-3-{5-[2-(2-formyl-3-hydroxy-5-methoxyphenoxy)acetamido]-2H-pyrazol-3-yl}cyclopentyl N-isopropylcarbamate as a white Solid (13.3 mg, 26.51%). MS (ESI) mass calcd. for C₂₂H₂₈N₄O₇, 460.19 m/z, found 461.15 [M+H]⁺. ¹H NMR (400 MHz, Methanol-d₄) δ 10.27 (s, 1H), 6.45-6.32 (m, 1H), 6.17-6.03 (m, 2H), 5.19-5.08 (m, 1H), 4.87-4.65 (m, 2H), 4.10-3.82 (m, 3H), 3.79-3.70 (m, 1H), 3.28-3.14 (m, 1H), 2.85-2.67 (m, 2H), 2.62-2.50 (m, 1H), 2.24-2.07 (m, 1H), 2.04-1.64 (m, 4H), 1.39-1.25 (m, 1H), 1.13 (t, J=5.6 Hz, 6H).

### Example 4.13: Synthesis of Compound 72: (1R,3S)-3-(3-(2-(2-formyl-3-hydroxy-5-(methoxymethyl)phenoxy)acetamido)-1H-pyrazol-5-yl)cyclopentyl isopropylcarbamate

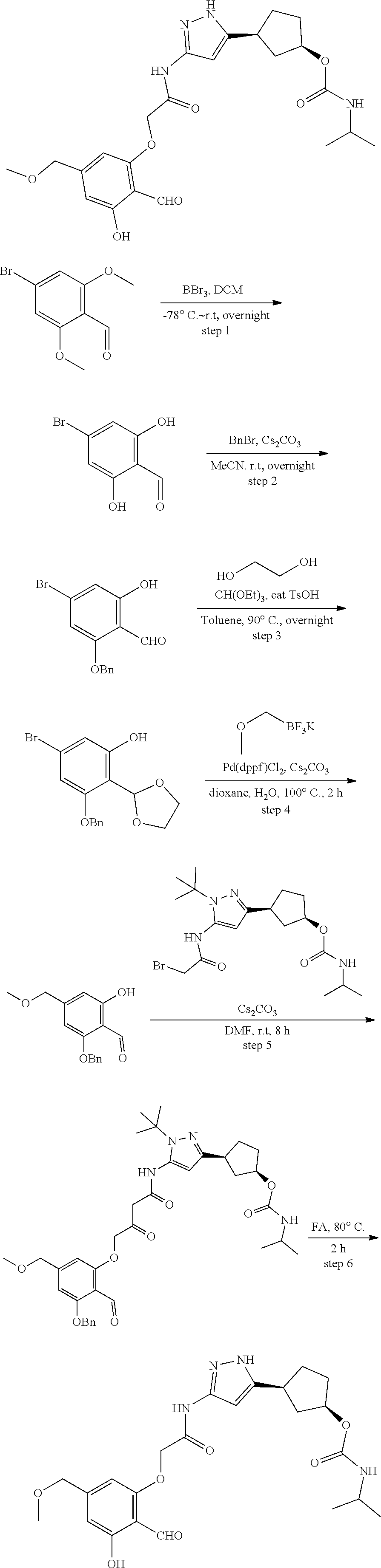

### Step 1: Synthesis of 4-bromo-2,6-dihydroxybenzaldehyde

To a stirred solution of 4-bromo-2,6-dimethoxybenzaldehyde (20.0 g, 81.609 mmol, 1 equiv) in DCM (100 mL) was added boron tribromide (489.6 mL, 489.654 mmol, 6.00 equiv, 1M in DCM) dropwise at −78° C. and stirred overnight at r.t under nitrogen atmosphere. Desired product could be detected by LCMS. The reaction mixture was then quenched with NaHCO₃ (200 mL) and extracted with DCM (200 mL×3), and the combined extracts washed with brine (200 mL), dried over anhydrous Na₂SO₄, filtered, and concentrated to dryness under vacuum. The residue was purified by silica gel column with EA/PE (0-30%) to afford 4-bromo-2,6-dihydroxybenzaldehyde (15.0 g, 80.04%) light yellow solid. MS (ESI) calcd. For C₇H₅BrO₃, 215.94 m/z, found 217.15 [M+H]⁺.

### Step 2: Synthesis of 2-(benzyloxy)-4-bromo-6-hydroxybenzaldehyde

To a solution of 4-bromo-2,6-dihydroxybenzaldehyde (15.0 g, 69.119 mmol, 1 equiv) and Cs₂CO₃ (45.0 g, 138.238 mmol, 2 equiv) in ACN (100 mL) was added benzyl bromide (11.8 g, 69.119 mmol, 1 equiv) dropwise at 0° C. and stirred overnight at r.t. The reaction mixture was then quenched with H₂O (200 mL) and extracted with EA (100 mL×3). The combined extracts washed with brine (100 mL), dried over anhydrous Na₂SO₄, filtered, and concentrated to dryness under vacuum. The residue was purified by silica gel chromatography EA/PE (0-30%) to afford 2-(benzyloxy)-4-bromo-6-hydroxybenzaldehyde (15.0 g, 70.66%) as yellow solid. MS (ESI) mass calcd. For C₁₄H₁₁BrO₃, 305.99 m/z, found 307.15 [M+H]⁺.

### Step 3: Synthesis of 3-(benzyloxy)-5-bromo-2-(1,3-dioxolan-2-yl)phenol

To a stirred solution of 2-(benzyloxy)-4-bromo-6-hydroxybenzaldehyde (7.0 g, 22.791 mmol, 1 equiv), triethyl orthoformate (10.1 g, 68.373 mmol, 3 equiv) and ethylene glycol (7.1 g, 113.955 mmol, 5 equiv) in toluene (50 mL) was added TsOH (392.4 mg, 2.279 mmol, 0.1 equiv) and stirred overnight at 90° C. under nitrogen atmosphere. Desired product could be detected by LCMS. The reaction mixture was then quenched with H₂O (100 mL) and extracted with EA (50 mL×3), and the combined extracts washed with brine (50 mL), dried over anhydrous Na₂SO₄, filtered, and concentrated to dryness under vacuum. The residue was purified by silica gel chromatography EA/PE (0-30%) to afford 3-(benzyloxy)-5-bromo-2-(1,3-dioxolan-2-yl)phenol (5.0 g, 62.47%) as a yellow oil. MS (ESI) mass calcd. For C₁₆H₁₅BrO₄, 350.02 m/z, found 351.15 [M+H]⁺.

### Step 4: Synthesis of 2-(benzyloxy)-6-hydroxy-4-(methoxymethyl)benzaldehyde

To a solution of 3-(benzyloxy)-5-bromo-2-(1,3-dioxolan-2-yl)phenol (500.0 mg, 1.424 mmol, 1 equiv), Pd(dppf)Cl₂ (816.1 mg, 4.272 mmol, 3 equiv) and Cs₂CO₃ (1.2 g, 3.560 mmol, 2.5 equiv) in 1,4-dioxane (10 mL) and H₂O (2 mL) was added potassium methoxy-methyltrifluoroborate (302.5 mg, 1.581 mmol, 1.5 equiv) and stirred for 2 h at 100° C. under nitrogen atmosphere. Desired product could be detected by LCMS. The reaction mixture was then quenched with H₂O (20 mL) and extracted with EA (10 mL×3), and the combined extracts washed with brine (10 mL), dried over anhydrous Na₂SO₄, filtered, and concentrated to dryness under vacuum. The residue was purified by silica gel column chromatography, eluted with EA/PE (0-30%) to afford 2-(benzyloxy)-6-hydroxy-4-(methoxymethyl)benzaldehyde (100.0 mg, 25.79%) as a yellow solid. MS (ESI) calcd. For C₁₆H₁₆O₄, 272.10 m/z, found 273.15 [M+H]⁺.

### Step 5: Synthesis of (1R,3S)-3-(5-(2-(3-(benzyloxy)-2-formyl-5-(methoxymethyl)phenoxy) acetamido)-1-(tert-butyl)-1H-pyrazol-3-yl)cyclopentyl isopropylcarbamate

2-(Benzyloxy)-6-hydroxy-4-(methoxymethyl)benzaldehyde (68.5 mg, 0.252 mmol, 1.2 equiv), Cs₂CO₃ (78.3 mg, 3.560 mmol, 2.5 equiv) were dissolved in DMF (10 mL). Then, (1R,3S)-3-(5-(2-bromoacetamido)-1-(tert-butyl)-1H-pyrazol-3-yl)cyclopentyl isopropylcarbamate (90.0 mg, 0.210 mmol, 1.00 equiv) was added to the reaction flask and stirred for 8 h at r.t. Desired product could be detected by LCMS. The reaction mixture was then quenched with H₂O (20 mL) and extracted with EA (10 mL×3), and the combined extracts washed with brine (10 mL), dried over anhydrous Na₂SO₄, filtered, and concentrated to dryness under vacuum. The residue was purified by silica gel column chromatography, eluted with MeOH/DCM (0-10%) to afford (1R,3S)-3-(5-{2-[3-(benzyloxy)-2-formyl-5-(methoxymethyl)phenoxy]acetamido}-1-tert-butylpyrazol-3-yl)cyclopentyl N-isopropylcarbamate (65.0 mg, 49.95%) as a yellow oil. MS (ESI) calcd. For C₃₄H₄₄N₄O₇, 620.32 m/z, found 621.15 [M+H]⁺.

### Step 6: Synthesis of (1R,3S)-3-(3-(2-(2-formyl-3-hydroxy-5-(methoxymethyl)phenoxy) acetamido)-1H-pyrazol-5-yl)cyclopentyl isopropylcarbamate

(1R,3S)-3-(5-{2-[3-(Benzyloxy)-2-formyl-5-(methoxymethyl)phenoxy]acetamido}-1-tert-butylpyrazol-3-yl)cyclopentyl N-isopropylcarbamate (65.0 mg, 0.113 mmol, 1 equiv) was dissolved in FA (1 mL) and stirred for 2 h at 80° C. Desired product could be detected by LCMS. The resulting mixture was concentrated under reduced pressure and purified by Prep-HPLC with the following conditions: Column: Xbridge Shield RP18 OBD Column, 30×150 mm, 5 um; Mobile Phase A: Water (0.1% FA), Mobile Phase B: ACN; Flow rate: 60 mL/min; Gradient: 26% B to 51% B in 10 min; Wave Length: 254/220 nm; RT1 (min): 8.47. This resulted in (1R,3S)-3-(5-{2-[2-formyl-3-hydroxy-5-(methoxymethyl)phenoxy]acetamido}-2H-pyrazol-3-yl)cyclopentyl N-isopropylcarbamate (7.2 mg, 12.97%) as a white solid. MS (ESI) calcd. For C₂₃H₃₀N₄O₇, 474.21 m/z, found 475.15 [M+H]⁺. ¹H NMR (300 MHz, DMSO-d₆) δ (ppm): 12.15 (s, 1H), 10.49 (s, 1H), 10.35 (s, 1H), 6.91 (d, J=7.8 Hz, 1H), 6.49 (d, J=7.9 Hz, 2H), 6.31 (s, 1H), 4.98 (s, 1H), 4.81 (s, 2H), 4.38 (s, 2H), 3.30 (s, 3H), 3.12 (s, 2H), 2.00-1.73 (s, 3H), 1.68 (d, J=7.7 Hz, 2H), 1.58 (s, 1H), 1.02 (d, J=6.6 Hz, 6H).

### Example 4.14: Synthesis of Compound 73: (1R,3S)-3-(3-(2-(5-chloro-2-formyl-3-hydroxyphenoxy)acetamido)-1H-pyrazol-5-yl)cyclopentyl isopropylcarbamate

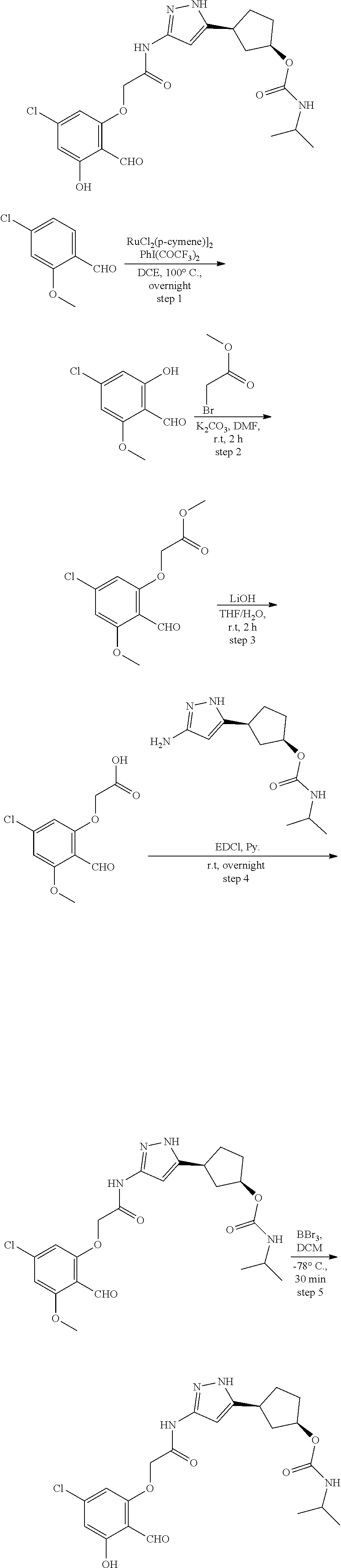

### Step 1: Synthesis of 4-chloro-2-hydroxy-6-methoxybenzaldehyde

A solution of 4-chloro-2-methoxybenzaldehyde (2.0 g 11.724 mmol, 1 equiv) in DCE (30 mL) was treated with (bis(trifluoroacetoxy)iodo)benzene (9.3 g, 23.448 mmol, 2 equiv), [RuCl₂(p-cymene)]₂ (815.6 mg, 1.332 mmol, 0.10 equiv) and stirred overnight at 100° C. under nitrogen atmosphere. The reaction mixture was then treated with H₂O (60 mL) and extracted with DCM (30 mL×3), and the combined extracts washed with brine (30 mL), dried over anhydrous Na₂SO₄, filtered, and concentrated to dryness under vacuum. The residue was purified by silica gel column chromatography, eluted with PE/EA (0-16%) to afford 4-chloro-2-hydroxy-6-methoxy benzaldehyde (400.0 mg, 18.29%) as a yellow oil. MS (ESI) calcd. For C₈H₇ClO₃, 186.06 m/z, found 185.15 [M−H]⁻.

### Step 2: Synthesis of methyl 2-(5-chloro-2-formyl-3-methoxyphenoxy)acetate

To a solution of 4-chloro-2-hydroxy-6-methoxybenzaldehyde (120.0 mg, 0.643 mmol, 1 equiv) and K₂CO₃ (133.3 mg, 0.965 mmol, 1.5 equiv) in DMF (3 mL) was added methyl 2-bromoacetate (150.0 mg, 0.772 mmol, 1.2 equiv) and stirred for 2 h at rt. The reaction mixture was then treated with H₂O (6 mL) and extracted with EA (3 mL×3), and the combined extracts washed with brine (3 mL), dried over anhydrous Na₂SO₄, filtered, and concentrated to dryness under vacuum. The residue was purified by silica gel column chromatography, eluted with PE/EA (0-40%) to afford methyl 2-(5-chloro-2-formyl-3-methoxyphenoxy)acetate (112.0 mg, 67.33%) as a yellow solid. MS (ESI) calcd. For C₁H₁₁ClO₅, 258.02 m/z, found 259.15 [M+H]⁺.

### Step 3: Synthesis of 2-(5-chloro-2-formyl-3-methoxyphenoxy)acetic acid

A solution of methyl 2-(5-chloro-2-formyl-3-methoxyphenoxy)acetate (100.0 mg, 0.387 mmol, 1 equiv) in THF (6 mL) and H₂O (2 mL) was treated with LiOH (23.2 mg, 0.968 mmol, 2.5 equiv) and stirred for 2 h at rt. The mixture was acidified to pH 6 with HCl (2 mol/L) followed by extraction with EA (5 mL×3), the combined extracts washed with brine (3 mL), dried over anhydrous Na₂SO₄, filtered, and concentrated to dryness under vacuum. The resulting mixture was concentrated under reduced pressure to afford 5-chloro-2-formyl-3-methoxyphenoxyacetic acid (80.1 mg, 84.58%) as a white solid. MS (ESI) calcd. For C₁₀H₉ClO₅, 244.01 m/z, found 245.15 [M+H]⁺.

### Step 4: Synthesis of (1R,3S)-3-(3-(2-(5-chloro-2-formyl-3-methoxyphenoxy)acetamido)-1H-pyrazol-5-yl)cyclopentyl isopropylcarbamate

A solution of 5-chloro-2-formyl-3-methoxyphenoxyacetic acid (69.8 mg, 0.286 mmol, 1.2 equiv) and (1R,3S)-3-(5-amino-2H-pyrazol-3-yl)cyclopentyl N-isopropylcarbamate (60.0 mg, 0.238 mmol, 1.00 equiv) in pyridine (5 mL) was treated with EDCI (68.4 mg, 0.357 mmol, 1.5 equiv) and stirred overnight at rt. The reaction mixture was then treated with H₂O (10 mL) and extracted with EA (5 mL×3), the combined extracts were washed with brine (5 mL), dried over anhydrous Na₂SO₄, filtered, and concentrated to dryness under vacuum. The residue was purified by silica gel column chromatography, eluted with MeOH/DCM (0-10%) to afford (1R,3S)-3-{5-[2-(5-chloro-2-formyl-3-methoxyphenoxy)acetamido]-2H-pyrazol-3-yl}cyclopentyl N-isopropyl carbamate (50.0 mg, 43.90%) as a white solid. MS (ESI) calcd. For C₂₂H₂₇ClN₄O₆, 418.16 m/z, found 419.15 [M+H]⁺.

### Step 5: Synthesis of (1R,3S)-3-(3-(2-(5-chloro-2-formyl-3-hydroxyphenoxy)acetamido)-1H-pyrazol-5-yl)cyclopentyl isopropylcarbamate

A solution of (1R,3S)-3-{5-[2-(5-chloro-2-formyl-3-methoxyphenoxy)acetamido]-2H-pyrazol-3-yl}cyclopentyl N-isopropylcarbamate (35.0 mg, 0.073 mmol, 1 equiv) in DCM (2 mL) was cooled to −78° C. and treated with boron tribromide (0.02 mL, 0.219 mmol, 6 equiv, 1M in DCM), the resulting solution was stirred for 30 mins at −78° C. under nitrogen atmosphere. The reaction mixture was then treated with NaHCO₃ (3 mL) and extracted with DCM (5 mL×3). The combined extracts were washed with brine (3 mL), dried over anhydrous Na₂SO₄, filtered, and concentrated to dryness under vacuum. The crude product was then purified by Prep-HPLC with the following conditions: Column: Xselect CSH Prep C18 OBD Column, 30×150 nm, 5 um; Mobile Phase A: H₂O (0.1% FA), Mobile Phase B: ACN; Flow rate: 60 mL/min; Gradient: 26% B to 48% B in 10 mins; Wave Length: 254 nm/220 nm. This resulted in (1R,3S)-3-{5-[2-(5-chloro-2-formyl-3-hydroxyphenoxy)acetamido]-2H-pyrazol-3-yl}cyclopentyl N-isopropylcarbamate (1.6 mg, 4.48%) as a yellow solid. MS (ESI) calcd. For C₂₁H₂₅ClN₄O₆, 464.15 m/z, found 465.15 [M+H]⁺. ¹H NMR (300 MHz, DMSO-d₆) δ (ppm): 12.18 (s, 1H), 10.53 (s, 1H), 9.78 (s, 1H), 6.96 (s, 1H), 6.31 (s, 1H), 6.15 (s, 1H), 5.81 (s, 1H), 5.01 (s, 1H), 4.72 (s, 1H), 3.61-3.54 (m, 2H), 3.51 (s, 1H), 3.04 (d, J=4.8 Hz, 1H), 2.49 (m, 1H), 2.01 (m, 1H), 1.93-1.82 (m, 1H), 1.71 (d, J=9.2 Hz, 2H), 1.58 (m, 1H), 1.02 (d, J=6.5 Hz, 6H).

### Example 4.15: Synthesis of Compound 81: (1R,3S)-3-{5-[2-(2-formyl-3-hydroxyphenoxy)acetamido]-2H-pyrazol-3-yl}cyclopentyl N-propylcarbamate; trifluoroacetic acid

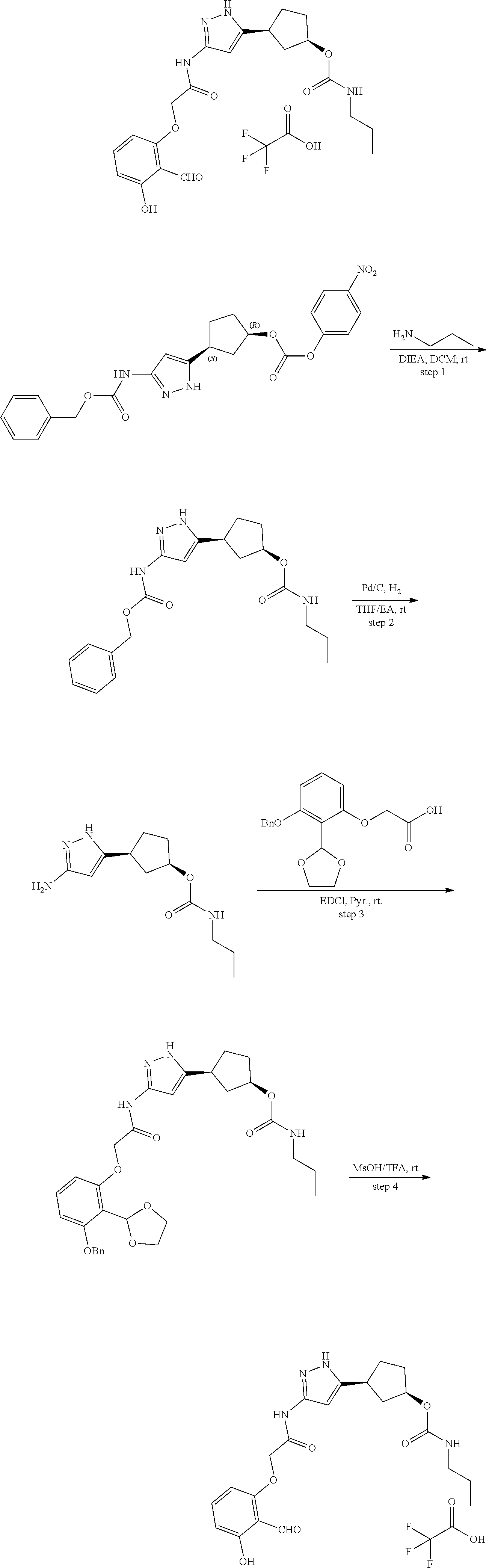

### Step 1: Synthesis of benzyl N-{5-[(1S,3R)-3-[(propylcarbamoyl)oxy]cyclopentyl]-1H-pyrazol-3-yl}carbamate

To a solution of (1R,3S)-3-(5-{[(benzyloxy)carbonyl]amino}-2H-pyrazol-3-yl)cyclopentyl 4-nitrophenyl carbonate (1.2 g, 2.573 mmol, 1 equiv) in DCM (10 mL) was added propylamine (0.3 g, 5.146 mmol, 2 equiv), DIEA (1.7 g, 12.865 mmol, 5 equiv) and the mixture was stirred for 1 h at room temperature. The reaction mixture was quenched by NaOH (0.5 M, 50 mL) and extracted with DCM (3×25 mL), and the combined extracts washed with brine (50 mL), dried over anhydrous Na₂SO₄, filtered, and concentrated to dryness in vacuo to give a nearly yellow viscous oil. The residue was purified by reversed-phase flash chromatography with the following conditions: column, C18 silica gel; mobile phase, H₂O (10 mM NH₄HCO₃) in ACN, 10% to 95% gradient in 35 min; detector, UV 254 nm. This resulted in benzyl N-{5-[(1S,3R)-3-[(propylcarbamoyl)oxy]cyclopentyl]-1H-pyrazol-3-yl}carbamate (900.0 mg, 90.53%) as a white solid. MS (ESI) calcd. for C₂₀H₂₆N₄O₄, 386.20 m/z, found 387.10 [M+H]⁺.

### Step 2: Synthesis of (1R,3S)-3-(5-amino-2H-pyrazol-3-yl)cyclopentyl N-propylcarbamate

To a solution of benzyl N-{5-[(1S,3R)-3-[(propylcarbamoyl)oxy]cyclopentyl]-1H-pyrazol-3-yl}carbamate (500.0 mg, 1.294 mmol, 1 equiv) in THF (10 mL) and EA (10 mL) was added Pd/C (10%, 500.0 mg) in a 100 mL bottle flask. The mixture was hydrogenated at room temperature for 1 h under hydrogen atmosphere using a hydrogen balloon, followed by filteration through a Celite pad and concentrated under reduced pressure. This resulted in (1R,3S)-3-(5-amino-2H-pyrazol-3-yl)cyclopentyl N-propylcarbamate (300 mg, 91.09%) as a pink solid. MS (ESI) calcd. for C₁₂H₂₀N₄O₂, 252.16 m/z, found 253.10 [M+H]⁺.

### Step 3: Synthesis of (1R,3S)-3-(5-{2-[3-(benzyloxy)-2-(1,3-dioxolan-2-yl)phenoxy]acetamido}-2H-pyrazol-3-yl)cyclopentyl N-propylcarbamate

To a stirred solution of (1R,3S)-3-(5-amino-2H-pyrazol-3-yl)cyclopentyl N-propylcarbamate (300.0 mg, 1.189 mmol, 1 equiv) in anhydrous pyridine (4 mL) was added EDCI (541.10 mg, 1.427 mmol, 1.2 equiv). The reaction mixture was stirred at room temperature for a period of 1 h. The resulting mixture was diluted with water and extracted with EA (3×100 ml). The combined organic layers were washed with brine (3×100 ml), dried over anhydrous Na₂SO₄. After filtration, the filtrate was concentrated under reduced pressure. The residue was purified by reversed-phase flash chromatography with the following conditions: column, C18 silica gel; mobile phase, H₂O (5% NH₄HCO₃) in ACN, 10% to 95% gradient in 35 min; detector, UV 254 nm. This resulted in (1R,3S)-3-(5-{2-[3-(benzyloxy)-2-(1,3-dioxolan-2-yl)phenoxy]acetamido}-2H-pyrazol-3-yl) cyclopentyl N-propylcarbamate (80.0 mg, 11.92%) as a yellow solid. MS (ESI) calcd. for C₃₀H₃₆N₄O₇, 564.26 m/z, found 565.35 [M+H]⁺.

### Step 4: Synthesis of (1R,3S)-3-{5-[2-(2-formyl-3-hydroxyphenoxy)acetamido]-2H-pyrazol-3-yl}cyclopentyl N-propylcarbamate; trifluoroacetic acid

To a stirred solution of (1R,3S)-3-(5-{2-[3-(benzyloxy)-2-(1,3-dioxolan-2-yl)phenoxy]acetamido}-2H-pyrazol-3-yl)cyclopentyl N-propylcarbamate (60.0 mg, 0.106 mmol, 1 equiv) in TFA (1.5 mL) were added MsOH (0.5 mL) dropwise at room temperature. The resulting mixture was stirred for 0.5 h. The reaction was monitored by LCMS. The resulting mixture was concentrated under reduced pressure. The crude product was purified by Prep-HPLC with the following conditions (Column: Xselect CSH Prep C18 OBD Column, 30×150 mm, 5 um; Mobile Phase A: Water (0.1% FA), Mobile Phase B: ACN; Flow rate: 60 mL/min; Gradient: 25% B to 47% B in 10 min; Wave Length: 254/220 nm) to afford (1R,3S)-3-{5-[2-(2-formyl-3-hydroxyphenoxy)acetamido]-2H-pyrazol-3-yl}cyclopentyl N-propylcarbamate; trifluoroacetic acid (7.7 mg, 12.86%) as a white solid. MS(ESI) calcd for C₂₃H₂₇F₃N₄O₈, 430.19 m/z, found 431.20 [M+H]⁺. ¹H NMR (400 MHz, DMSO-d₆) δ 11.71 (br s, 1H), 10.55 (s, 1H), 10.40 (s, 1H), 7.48-7.52 (m, 1H), 7.03-7.06 (m, 1H), 6.51-6.57 (m, 2H), 6.31 (s, 1H), 4.99-5.00 (m, 1H), 4.82 (s, 2H), 3.02-3.07 (m, 1H), 2.88-2.93 (m, 2H), 2.45-2.48 (m, 1H), 1.90-2.01 (m, 1H), 1.86-1.89 (m, 1H), 1.70-1.75 (m, 2H), 1.58-1.59 (m, 1H), 1.35-1.39 (m, 2H), 0.79-0.83 (m, 3H). ¹⁹F NMR (376 MHz, DMSO-d₆) δ −74.93.

### Example 4.16: Synthesis of Compound 82: (1R,3S)-3-{5-[2-(2-formyl-3-hydroxyphenoxy)acetamido]-2H-pyrazol-3-yl}cyclopentyl N-(1-methylcyclopropyl)carbamate; trifluoroacetic acid

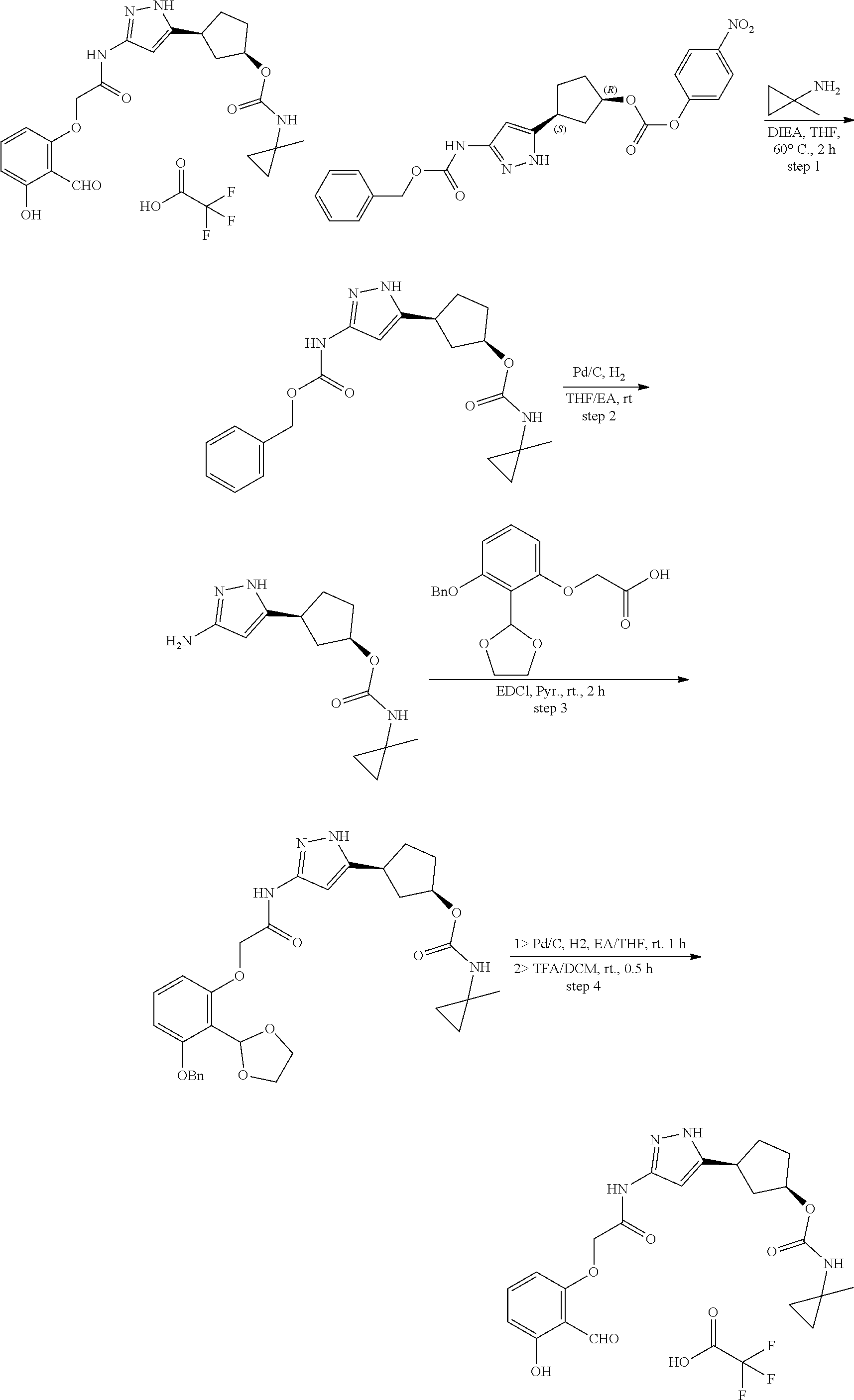

### Step 1: Synthesis of (1R,3S)-3-(3-(((benzyloxy)carbonyl)amino)-1H-pyrazol-5-yl)cyclopentyl (1-methylcyclopropyl)carbamate

To a stirred solution of (1R,3S)-3-(5-{[(benzyloxy)carbonyl]amino}-2H-pyrazol-3-yl)cyclopentyl 4-nitrophenyl carbonate (1.2 g, 2.573 mmol, 1 equiv) and 1-methylcyclopropan-1-amine hydrochloride (0.55 g, 5.146 mmol, 2 equiv) in THF (20 mL) was added DIEA (1.66 g, 12.865 mmol, 5 equiv) dropwise at 60° C. The resulting mixture was stirred for 2 h at 60° C. The reaction mixture was then treated with H₂O (50 mL), dropwise over 10 min, extracted with EtOAc (50 mL×2), and the combined extracts washed with brine (50 mL), dried over anhydrous Na₂SO₄, filtered, and concentrated to dryness in vacuo to give a nearly yellow viscous oil. The oil was purified by reversed-phase flash chromatography with the following conditions: column, C18 silica gel; mobile phase, water in ACN, 10% to 50% gradient in 10 min; detector, UV 220 nm. This resulted in (1R,3S)-3-(5-{[(benzyloxy)carbonyl]amino}-2H-pyrazol-3-yl)cyclopentyl N-(1-methylcyclopropyl)carbamate (720.0 mg, 54.97%) as a white solid. MS (ESI) calcd. for C₂₁H₂₆N₄O₄, 398.20 m/z, found 399.20 [M+H]⁺.

### Step 2: Synthesis of (1R,3S)-3-(3-amino-1H-pyrazol-5-yl)cyclopentyl (1-methylcyclopropyl) carbamate

To a solution of (1R,3S)-3-(3-(((benzyloxy)carbonyl)amino)-1H-pyrazol-5-yl)cyclopentyl (1-methylcyclopropyl)carbamate (700.0 mg, 1.757 mmol, 1 equiv) in EA (10 mL) and THF (10 mL) was added Pd/C (500 mg, 10% wt) in a pressure tank. The mixture was hydrogenated at room temperature under 30 psi of hydrogen pressure for 1 h, filtered through a Celite pad and concentrated under reduced pressure to give (1R,3S)-3-(3-(((benzyloxy)carbonyl)amino)-1H-pyrazol-5-yl)cyclopentyl (1-methylcyclopropyl)carbamate (450.0 mg, 96.91%) as an off-white solid. MS (ESI) calcd. for C₁₃H₂₀N₄O₂, 264.16 m/z, found 265.20 [M+H]⁺.

### Step 3: Synthesis of (1R,3S)-3-(3-(2-(3-(benzyloxy)-2-(1,3-dioxolan-2-yl)phenoxy)acetamido)-1H-pyrazol-5-yl)cyclopentyl (1-methylcyclopropyl)carbamate

To a stirred solution of (1R,3S)-3-(5-amino-2H-pyrazol-3-yl)cyclopentyl N-(1-methylcyclo-propyl)carbamate (96.0 mg, 0.363 mmol, 1.5 equiv) and 3-(benzyloxy)-2-(1,3-dioxolan-2-yl)phenoxyacetic acid (80.0 mg, 0.242 mmol, 1 equiv) in pyridine (3 mL) was added EDCI (69.6 mg, 0.363 mmol, 1.5 equiv) in portions at room temperature. The resulting mixture was stirred for 1 h at room temperature. The reaction mixture was then treated with H₂O (10 mL), dropwise over 10 min, extracted with EtOAc (10 mL×2), and the combined extracts washed with brine (10 mL), dried over anhydrous Na₂SO₄, filtered, and concentrated to dryness in vacuo to give a nearly yellow viscous oil. The oil was then subjected to silica gel chromatography (0-30% MeOH/DCM) to give (1R,3S)-3-(3-(2-(3-(benzyloxy)-2-(1,3-dioxolan-2-yl)phenoxy)acetamido)-1H-pyrazol-5-yl)cyclopentyl (1-methylcyclopropyl)carbamate (60.0 mg, 42.96%) as a yellow solid. MS (ESI) calcd. for C₃₁H₃₆N₄O₇, 576.26 m/z, found 577.35 [M+H]⁺.

### Step 4: Synthesis of (1R,3S)-3-{5-[2-(2-formyl-3-hydroxyphenoxy)acetamido]-2H-pyrazol-3-yl}cyclopentyl N-(1-methylcyclopropyl)carbamate; trifluoroacetic acid

To a solution of (1R,3S)-3-(5-{2-[3-(benzyloxy)-2-(1,3-dioxolan-2-yl)phenoxy]acetamido}-2H-pyrazol-3-yl)cyclopentyl N-(1-methylcyclopropyl)carbamate (40.0 mg, 0.069 mmol, 1 equiv) in EA (3 mL) and THF (3 mL) was added Pd/C (10%, 3.91 mg) in a pressure tank. The mixture was hydrogenated at room temperature under 30 psi of hydrogen pressure for 1 h, filtered through a Celite pad and concentrated under reduced pressure. The mixture is directly used for next step. The compound was resuspended in DCM (1 mL) and TFA (0.3 mL). The resulting mixture was stirred for 1 h at room temperature. The crude product was purified by Prep-HPLC with the following conditions (Column: XSelect CSH Fluoro Phenyl 30×150 mm, 5 μm; Mobile Phase A: Water (0.05% TFA), Mobile Phase B: ACN; Flow rate: 60 mL/min; Gradient: 22% B to 44% B in 10 min; Wave Length: 254 nm/220 nm; RT1 (min): 8.6) to afford (1R,3S)-3-{5-[2-(2-formyl-3-hydroxyphenoxy)acetamido]-2H-pyrazol-3-yl}cyclopentyl N-(1-methylcyclopropyl)carbamate; trifluoroacetic acid (1.5 mg, 3.86%) as a white solid. MS (ESI) calcd. for C₂₂H₂₆N₄O₆, 442.19 m/z, found 443.20 [M+H]⁺. ¹H NMR (400 MHz, DMSO-d₆) δ 11.66 (s, 1H), 10.72-10.76 (m, 1H), 10.06 (s, 1H), 7.84 (s, 1H), 7.59-7.64 (m, 1H), 6.95-7.12 (m, 2H), 6.91-6.93 (m, 1H), 6.33 (s, 1H), 4.97-5.02 (m, 1H), 4.17 (s, 2H), 3.55-3.57 (m, 1H), 3.03-3.07 (m, 1H), 2.44-2.46 (m, 1H), 1.96-2.02 (m, 1H), 1.86-1.91 (m, 1H), 1.68-1.72 (m, 2H), 1.55-1.59 (m, 1H), 1.01-1.03 (m, 6H). ¹⁹F NMR (376 MHz, DMSO-d₆) δ −74.46.

### Example 4.17: Synthesis of Compound 83: (1R,3S)-3-{5-[2-(2-formyl-3-hydroxyphenoxy) acetamido]-2H-pyrazol-3-yl}cyclopentyl N-(1-methylcyclobutyl) carbamate; trifluoroacetic acid

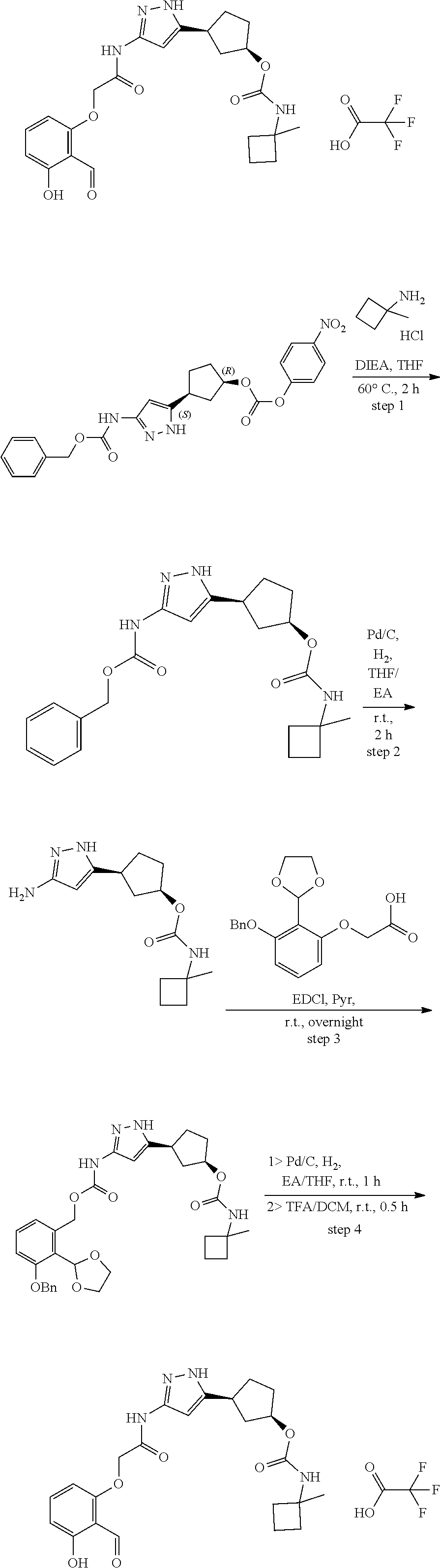

### Step 1: Synthesis of (1R,3S)-3-(3-(((benzyloxy)carbonyl)amino)-1H-pyrazol-5-yl)cyclopentyl (1-methylcyclobutyl)carbamate

To a stirred solution of (1R,3S)-3-(5-{[(benzyloxy)carbonyl]amino}-2H-pyrazol-3-yl)cyclopentyl 4-nitrophenyl carbonate (1.10 g, 2.358 mmol, 1 equiv) and 1-methylcyclobutan-1-amine hydrochloride (0.86 g, 7.074 mmol, 3 equiv) in THF (20 mL) was added DIEA (4.11 mL, 23.580 mmol, 10 equiv) dropwise at room temperature. The mixture was stirred at 60° C. for 4 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 60% to 70% gradient in 10 min; detector, UV 254 nm to afford (1R,3S)-3-(5-{[(benzyloxy)carbonyl]amino}-2H-pyrazol-3-yl)cyclopentyl N-(1-methyl cyclobutyl)carbamate (514.0 mg, 42.89%) as a white solid. MS (ESI) calcd. for C₂₂H₂₈N₄O₄, 412.21 m/z, found 413.20 [M+H]⁺.

### Step 2: Synthesis of (1R,3S)-3-(3-amino-1H-pyrazol-5-yl)cyclopentyl (1-methylcyclobutyl) carbamate

A stirred solution of (1R,3S)-3-(5-{[(benzyloxy)carbonyl]amino}-2H-pyrazol-3-yl)cyclopentyl N-(1-methylcyclobutyl)carbamate (500.0 mg, 1.212 mmol, 1 equiv) in THF (8 mL) and EA (8 mL) was added 10% Pd/C (500.0 mg, 0.470 mmol, 0.39 equiv) in portions at room temperature under hydrogen atmosphere for 2 h. The resulting mixture was filtered, the filter cake was washed with MeOH (3×10 mL). The filtrate was concentrated under reduced pressure to afford (1R,3S)-3-(5-amino-2H-pyrazol-3-yl)cyclopentyl N-(1-methylcyclobutyl)carbamate (300 mg, 78.92%) as a light yellow solid. MS (ESI) calcd. for C₁₄H₂₂N₄O₂, 278.17 m/z, found 279.10 [M+H]⁺.

### Step 3: Synthesis of (1R,3S)-3-(3-(2-(3-(benzyloxy)-2-(1,3-dioxolan-2-yl)phenoxy)acetamido)-1H-pyrazol-5-yl)cyclopentyl (1-methylcyclobutyl)carbamate

To a stirred solution of (1R,3S)-3-(5-amino-2H-pyrazol-3-yl)cyclopentyl N-(1-methylcyclobutyl) carbamate (109.5 mg, 0.394 mmol, 1 equiv) and 3-(benzyloxy)-2-(1,3-dioxolan-2-yl)phenoxyacetic acid (130.0 mg, 0.394 mmol, 1.00 equiv) in pyridine (2.5 mL) was added EDCI (113.2 mg, 0.591 mmol, 1.5 equiv) in portions and let stir at room temperature for 2 h. The reaction was quenched with water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mM NH₄HCO₃), 70% to 80% gradient in 10 min; detector, UV 254 nm to afford (1R,3S)-3-(5-{2-[3-(benzyloxy)-2-(1,3-dioxolan-2-yl)phenoxy]acetamido}-2H-pyrazol-3-yl)cyclopentyl N-(1-methylcyclobutyl)carbamate (200.0 mg, 69.80%) as a yellow solid. MS (ESI) calcd. for C₃₂H₃₈N₄O₇, 590.27 m/z, found 591.45 [M+H]⁺.

### Step 4: Synthesis of (1R,3S)-3-{5-[2-(2-formyl-3-hydroxyphenoxy)acetamido]-2H-pyrazol-3-yl}cyclopentyl N-(1-methylcyclobutyl)carbamate; trifluoroacetic acid

To a stirred solution of (1R,3S)-3-(5-{2-[3-(benzyloxy)-2-(1,3-dioxolan-2-yl)phenoxy]acetamido}-2H-pyrazol-3-yl)cyclopentyl N-(1-methylcyclobutyl)carbamate (80.0 mg, 0.135 mmol, 1 equiv) in EA (2 mL) THF (2 mL) was added Pd/C (80.0 mg, 0.075 mmol, 0.56 equiv, 10%) in portions at room temperature under hydrogen atmosphere for 2 h. The resulting mixture was filtered, the filter cake was washed with EtOAc (3×5 mL). The filtrate was concentrated under reduced pressure. The above mixture was resuspended in DCM (1.5 mL) and TFA (0.5 mL) was added dropwise and let stir for 1 h at room temperature. The crude product (50.0 mg) was purified by Prep-HPLC with the following conditions (Column: Xselect CSH Prep C18 OBD Column, 30×150 mm, 5 um; Mobile Phase A: Water (0.05% TFA), Mobile Phase B: ACN; Flow rate: 60 mL/min; Gradient: 33% B to 55% B in 10 min; Wave Length: 254/220 nm) to afford (1R,3S)-3-{5-[2-(2-formyl-3-hydroxyphenoxy)acetamido]-2H-pyrazol-3-yl}cyclopentyl N-(1-methylcyclobutyl)carbamate; trifluoroacetic acid (6.0 mg, 7.69%) as a white solid. MS (ESI) calcd. for C₂₃H₂₈N₄O₆, 456.20 m/z, found 457.25 [M+H]⁺. ¹H NMR (400 MHz, DMSO-d₆) δ 11.71 (s, 1H), 10.54 (s, 1H), 10.39 (s, 1H), 7.48-7.53 (m, 1H), 7.17 (s, 1H), 6.51-6.57 (m, 2H), 6.32 (s, 1H), 4.92-5.01 (m, 1H), 4.82 (s, 2H), 3.02-3.06 (m, 1H), 2.41-2.48 (m, 1H), 2.20-2.31 (m, 2H), 1.97-2.08 (m, 1H), 1.85-1.92 (m, 1H), 1.76-1.83 (m, 2H), 1.64-1.74 (m, 4H), 1.53-1.62 (m, 1H), 1.30 (s, 3H). ¹⁹F NMR (376 MHz, DMSO-d₆) δ −74.80.

### Example 4.18: Synthesis of Compound 84: (1R,3S)-3-(3-(2-(2-formyl-3-hydroxyphenoxy) acetamido)-1H-pyrazol-5-yl)cyclopentyl tert-butylcarbamate

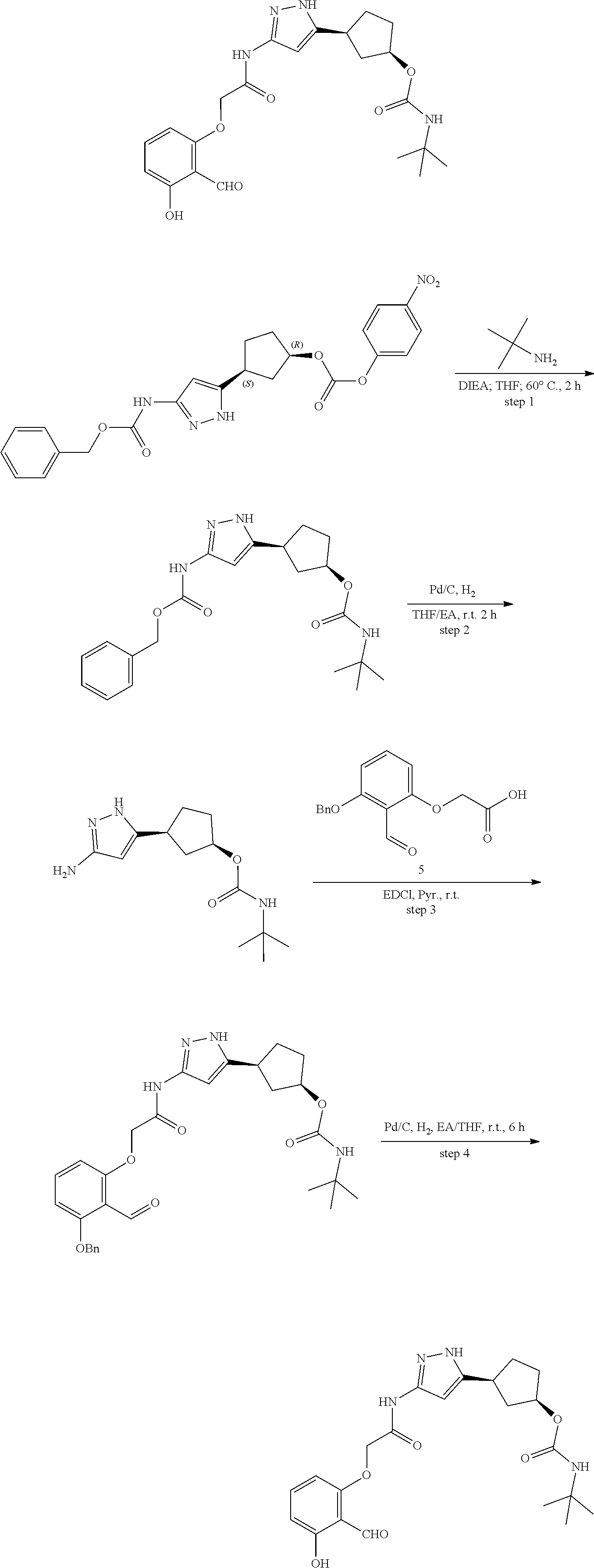

### Step 1: Synthesis of (1R,3S)-3-(3-(((benzyloxy)carbonyl)amino)-1H-pyrazol-5-yl)cyclopentyl tert-butylcarbamate

To a stirred solution of (1R,3S)-3-(5-{[(benzyloxy)carbonyl]amino}-2H-pyrazol-3-yl)cyclopentyl 4-nitrophenyl carbonate (0.8 g, 1.715 mmol, 1 equiv) and 2-methylpropan-2-amine (0.38 g, 5.145 mmol, 3 equiv) in THF (10 mL) was added DIEA (2.4 mL, 13.720 mmol, 8 equiv) in portions and heated for 2 h at 60° C. The resulting mixture was cooled to room temperature, diluted with water and extracted with DCM (3×40 mL). The combined organic layers were washed with NaOH (0.5 mol/L) (2×40 mL), dried over anhydrous Na₂SO₄. After filtration, the filtrate was concentrated under reduced pressure. The residue was purified by reversed-phase flash chromatography with the following conditions: column, C18 silica gel; mobile phase, ACN/H₂O (0.05% TFA), 5% to 80% gradient in 25 min; detector, UV 254 nm to afford (1R,3S)-3-(3-(((benzyloxy)carbonyl)amino)-1H-pyrazol-5-yl)cyclopentyl tert-butyl carbamate (360.0 mg, 40.46%) as a yellow solid. MS (ESI) calcd. for C₂₁H₂₈N₄O₄, 400.21 m/z, found 401.15 [M+H]⁺

### Step 2: Synthesis of (1R,3S)-3-(3-amino-1H-pyrazol-5-yl)cyclopentyl tert-butylcarbamate

To a stirred solution of benzyl N-{5-[(1S,3R)-3-[(tert-butylcarbamoyl)oxy]cyclopentyl]-1H-pyrazol-3-yl}carbamate (360.0 mg, 0.899 mmol, 1 equiv) in THF (3 mL) and EA (3 mL) was added 10% Pd/C (270.0 mg, 2.537 mmol, 2.82 equiv) in portions and let stir for for 2 h at room temperature under hydrogen atmosphere. The resulting mixture was filtered, the filter cake was washed with EA (4×10 mL). The filtrate was concentrated under reduced pressure to afford (1R,3S)-3-(5-amino-2H-pyrazol-3-yl)cyclopentyl N-tert-butylcarbamate (200.0 mg, 72.92%) as a brown oil. MS (ESI) calcd. for C₁₃H₂₂N₄O₂, 266.17 m/z, found 267.10 [M+H]⁺.

### Step 3: Synthesis of (1R,3S)-3-(3-(2-(3-(benzyloxy)-2-formylphenoxy)acetamido)-1H-pyrazol-5-yl)cyclopentyl tert-butylcarbamate

To a stirred mixture of (1R,3S)-3-(5-amino-2H-pyrazol-3-yl)cyclopentyl N-tert-butylcarbamate (150.0 mg, 0.563 mmol, 1 equiv) and 3-(benzyloxy)-2-formylphenoxyacetic acid (161.2 mg, 0.563 mmol, 1 equiv) in pyridine (4 mL) was added EDCI (161.9 mg, 0.844 mmol, 1.5 equiv) in portions and let stir for 2 h at room temperature. The resulting mixture was diluted with water and extracted with EA (3×20 mL). The combined organic layers were washed with water (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O/ACN (0.05% TFA), 5% to 95% gradient in 25 min; detector, UV 254 nm to afford (1R,3S)-3-(5-{2-[3-(benzyloxy)-2-formylphenoxy]acetamido}-2H-pyrazol-3-yl)cyclo pentyl N-tert-butylcarbamate (60.0 mg, 9.37%) as a light yellow solid. MS (ESI) calcd. For C₂₉H₃₄N₄O₆, 534.25 m/z, found 535.30 [M+H]⁺.

### Step 4: Synthesis of (1R,3S)-3-(3-(2-(2-formyl-3-hydroxyphenoxy)acetamido)-1H-pyrazol-5-yl)cyclopentyl tert-butylcarbamate

To a stirred solution of (1R,3S)-3-(5-{2-[3-(benzyloxy)-2-formylphenoxy]acetamido}-2H-pyrazol-3-yl)cyclopentyl N-tert-butylcarbamate (50.0 mg, 0.094 mmol, 1 equiv) in EA (2 mL, 20.430 mmol, 218.44 equiv) and THF (2 mL, 24.686 mmol, 263.94 equiv) was added 10% Pd/C (50.0 mg, 0.470 mmol, 5.02 equiv) in portions and let stir for 6 h at room temperature under hydrogen atmosphere. The resulting mixture was filtered, the filter cake was washed with EA (4×30 mL). The filtrate was concentrated under reduced pressure. The crude product was purified by Prep-HPLC with the following conditions (Column: XSelect CSH Fluoro Phenyl 30×150 mm, 5 μm; Mobile Phase A: water (0.05% TFA), Mobile Phase B: ACN; Flow rate: 60 mL/min; Gradient: 14% B to 38% B in 10 min; Wave Length: 254/220 nm; RT1 (min): 9 to afford (1R,3S)-3-{5-[2-(2-formyl-3-hydroxyphenoxy)acetamido]-2H-pyrazol-3-yl}cyclopentyl N-tert-butylcarbamate (3.0 mg, 7.05%) as a yellow solid. MS (ESI) calcd. for C₂₂H₂₈N₄O₆, 444.20 m/z, found 445.15 [M+H]⁺. 1H NMR (400 MHz, DMSO-d₆) δ 11.71 (s, 1H), 10.53 (s, 1H), 10.39 (s, 1H), 7.50 (t, J=8.4 Hz, 1H), 6.76 (s, 1H), 6.51-6.57 (m, 2H), 6.31 (s, 1H), 4.97 (s, 1H), 4.82 (s, 2H), 3.02-3.04 (m, 1H), 2.32-2.33 (m, 1H), 1.99-2.02 (m, 1H), 1.85-1.96 (m, 1H), 1.67-1.73 (m, 2H), 1.55-1.57 (m, 1H), 1.20 (s, 9H).

### Example 4.19: Synthesis of Compound 85: (1R,3S)-3-{5-[2-(2-formyl-3-hydroxyphenoxy) acetamido]-2H-pyrazol-3-yl}cyclopentyl pyrrolidine-1-carboxylate

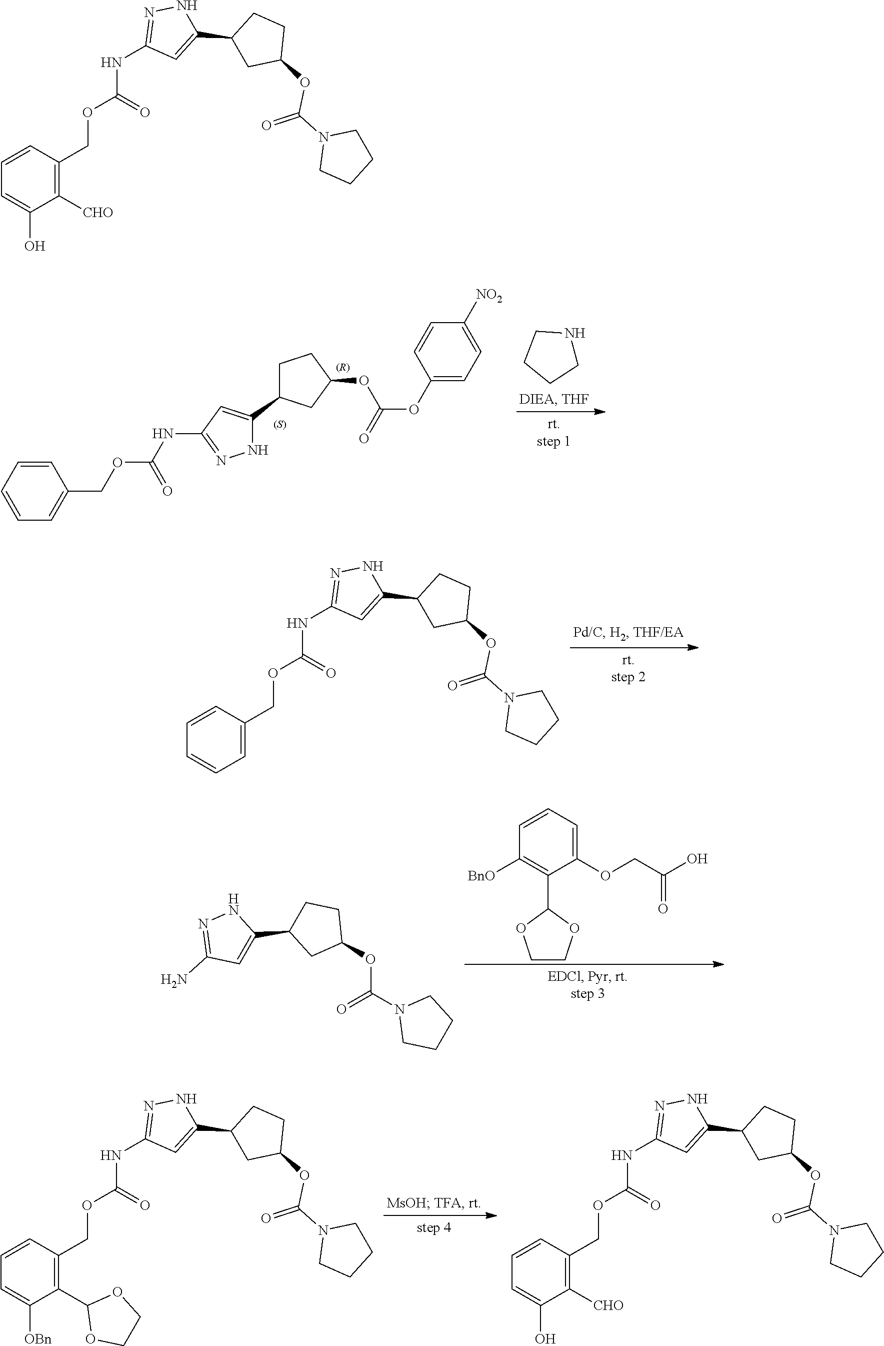

### Step 1: Synthesis of (1R,3S)-3-(5-{[(benzyloxy)carbonyl]amino}-2H-pyrazol-3-yl)cyclopentyl pyrrolidine-1-carboxylate

To a stirred solution of (1R,3S)-3-(5-{[(benzyloxy)carbonyl]amino}-2H-pyrazol-3-yl)cyclopentyl 4-nitrophenyl carbonate (1.2 g, 2.573 mmol, 1 equiv) and pyrrolidine (0.37 g, 5.146 mmol, 2 equiv) in THF (15 mL) were added DIEA (1.33 g, 10.292 mmol, 4 equiv) at 25° C. The resulting mixture was stirred for 12 h at 25° C. The resulting mixture was diluted with water and extracted with DCM (3×50 mL). The combined organic layers were washed with brine (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O/ACN, 45% gradient in 10 min; detector, UV 254 nm. This resulted in (1R,3S)-3-(5-{[(benzyloxy)carbonyl]amino}-2H-pyrazol-3-yl)cyclopentyl pyrrolidine-1-carboxylate (0.9 g, 85.63%) as a yellow solid. MS (ESI) calcd. for C₂₁H₂₆N₄O₄, 398.46 m/z, found 399.20 [M+H]⁺.

### Step 2: Synthesis of (1R,3S)-3-(5-amino-2H-pyrazol-3-yl)cyclopentyl pyrrolidine-1-carboxylate

To a stirred solution of (1R,3S)-3-(5-{[(benzyloxy)carbonyl]amino}-2H-pyrazol-3-yl)cyclopentyl pyrrolidine-1-carboxylate (850.0 mg, 2.133 mmol, 1 equiv) in THF (10 mL) and EA (10 mL) was added 10% Pd/C (700 mg) at 25° C. The resulting mixture was stirred for 12 h at 25° C. under H₂ atmosphere. The reaction was monitored by LCMS. The resulting mixture was filtered, the filter cake was washed with MeOH (2×100 mL). The filtrate was concentrated under reduced pressure. This resulted in (1R,3S)-3-(5-amino-2H-pyrazol-3-yl)cyclopentyl pyrrolidine-1-carboxylate (500.0 mg, 57.76%) as a yellow solid. MS (ESI) calcd. for C₁₃H₂₀N₄O₂, 264.33 m/z, found 265.20 [M+H]⁺.

### Step 3: Synthesis of (1R,3S)-3-(5-{2-[3-(benzyloxy)-2-(1,3-dioxolan-2-yl)phenoxy]acetamido}-2H-pyrazol-3-yl)cyclopentyl pyrrolidine-1-carboxylate

To a stirred solution of (1R,3S)-3-(5-amino-2H-pyrazol-3-yl)cyclopentyl pyrrolidine-1-carboxylate (96.1 mg, 0.364 mmol, 1 equiv) and 3-(benzyloxy)-2-(1,3-dioxolan-2-yl)phenoxyacetic acid (120.10 mg, 0.364 mmol, 1 equiv) in pyridine (4 mL) were added EDCI (104.54 mg, 0.546 mmol, 1.5 equiv) at 25° C. The resulting mixture was stirred for 3 h at 25° C. The reaction was monitored by LCMS. The resulting mixture was diluted with water and extracted with EA (3×15 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O (5 mM NH₄HCO₃5)/ACN, 50% gradient in 10 min; detector, UV 254 nm. This resulted in (1R,3S)-3-(5-{2-[3-(benzyloxy)-2-(1,3-dioxolan-2-yl)phenoxy]acetamido}-2H-pyrazol-3-yl)cyclopentyl pyrrolidine-1-carboxylate (80 mg, 28.96%) as a white solid. MS (ESI) calcd. for C₃₁H₃₆N₄O₇, 576.65 m/z, found 575.20 [M−H]⁻.

### Step 4: Synthesis of (1R,3S)-3-{5-[2-(2-formyl-3-hydroxyphenoxy)acetamido]-2H-pyrazol-3-yl}cyclopentyl pyrrolidine-1-carboxylate; trifluoroacetic acid

To a stirred solution of (1R,3S)-3-(5-{2-[3-(benzyloxy)-2-(1,3-dioxolan-2-yl)phenoxy]acetamido}-2H-pyrazol-3-yl)cyclopentyl pyrrolidine-1-carboxylate (60.0 mg, 0.104 mmol, 1 equiv) in TFA (1 mL) was added methanesulfonic acid (0.3 mL) at 25° C. The resulting mixture was stirred for 1 h at 25° C. The reaction was monitored by LCMS. The mixture was neutralized to pH 7 with NaOH (0.5 M). The resulting mixture was extracted with EA (3×20 mL). The combined organic layers were washed with brine (2×20 mL), dried over anhydrous Na₂SO₄. After filtration, the filtrate was concentrated under reduced pressure. The crude product was purified by Prep-HPLC with the following conditions (Column: YMC Triart C18 ExRs, 30×150 mm, 5 μm; Mobile Phase A: Water (0.05% TFA), Mobile Phase B: ACN; Flow rate: 60 mL/min; Gradient: 23% B to 46% B in 10 min; Wave Length: 254/220 nm) to afford (1R,3S)-3-{5-[2-(2-formyl-3-hydroxyphenoxy)acetamido]-2H-pyrazol-3-yl}cyclopentyl pyrrolidine-1-carboxylate (10.0 mg, 17.11%) as a white solid. MS (ESI) calcd. For C₂₄H₂₇F₃N₄O₈, 442.19 m/z, found 443.25 [M+H]⁺. ¹H NMR (300 MHz, DMSO-d₆) δ 11.69 (br s, 1H), 10.52 (s, 1H), 10.38 (s, 1H), 7.46-7.52 (m, 1H), 6.49-6.56 (m, 2H), 6.31 (s, 1H), 4.98-5.01 (m, 1H), 4.81 (s, 2H), 3.17-3.21 (m, 4H), 3.06-3.09 (m, 1H), 2.34-2.41 (m, 1H), 1.97-2.02 (m, 1H), 1.86-1.90 (m, 1H), 1.65-1.80 (m, 7H). ¹⁹F NMR (282 MHz, DMSO-d₆) δ −74.92.

### Example 4.20: Synthesis of Compound 86: (1R,3S)-3-(3-(2-(2-formyl-3-hydroxyphenoxy) acetamido)-1H-pyrazol-5-yl)cyclopentyl cyclopropylcarbamate

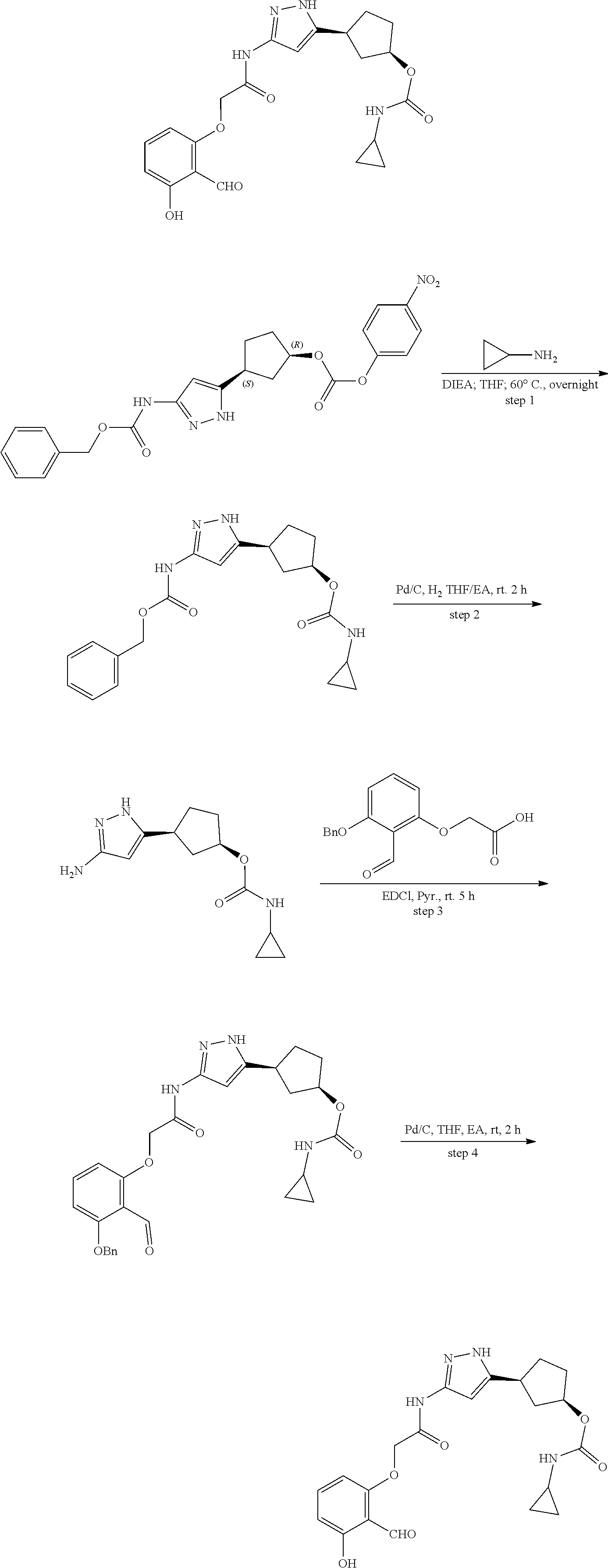

### Step 1: Synthesis of benzyl (5-((1S,3R)-3-((cyclopropylcarbamoyl)oxy)cyclopentyl)-1H-pyrazol-3-yl)carbamate

To a solution of (1R,3S)-3-(5-{[(benzyloxy)carbonyl]amino}-2H-pyrazol-3-yl)cyclopentyl 4-nitrophenyl carbonate (1.1 g, 2.358 mmol, 1 equiv) in THF (10 mL) was added DIEA (2 mL, 11.482 mmol, 4.87 equiv) and aminocyclopropane (0.4 g, 7.074 mmol, 3 equiv). After stirring for 2 h at 60° C., the resulting mixture was cooled to room temperature, diluted with water, and extracted with EA (3×30 mL). The combined organic layers were washed with sat. NaOH (2×50 mL). After filtration, the filtrate was concentrated under reduced pressure. The crude product was purified by reverse flash chromatography, eluted with H₂O (0.05% NH₄HCO₃):ACN (1:1) to afford (1R,3S)-3-(5-{[(benzyloxy)carbonyl]amino}-2H-pyrazol-3-yl)cyclopentyl N-cyclopropylcarbamate (500.0 mg, 50.89%) as a yellow solid. MS (ESI) calcd. for C₂₀H₂₄N₄O₄, 384.18 m/z, found 385.10 [M+H]⁺.

### Step 2: Synthesis of (1R,3S)-3-(3-amino-1H-pyrazol-5-yl)cyclopentyl cyclopropylcarbamate

To a solution of (1R,3S)-3-(5-{[(benzyloxy)carbonyl]amino}-2H-pyrazol-3-yl)cyclopentyl N-cyclopropylcarbamate (500.0 mg, 1.301 mmol, 1 equiv) in EA (10 mL)/THF (10 mL) was added 10% Pd/C (498.2 mg, 0.468 mmol, 0.36 equiv). After stirring for 4 h at room temperature under H₂ atmosphere, the resulting mixture was filtrated, the filtrate was concentrated under reduced pressure to afford (1R,3S)-3-(5-amino-2H-pyrazol-3-yl)cyclopentyl N-cyclopropylcarbamate (300.0 mg, 56.86%) as a yellow solid. MS (ESI) calcd. for C₁₂H₁₈N₄O₂, 250.14 m/z, found 251.15 [M+H]⁺.

### Step 3: Synthesis of (1R,3S)-3-(3-(2-(3-(benzyloxy)-2-formylphenoxy)acetamido)-1H-pyrazol-5-yl)cyclopentyl cyclopropylcarbamate

To a solution of (1R,3S)-3-(5-amino-2H-pyrazol-3-yl)cyclopentyl N-cyclopropylcarbamate (160.0 mg, 0.639 mmol, 1 equiv) in pyridine (5 mL) was added 3-(benzyloxy)-2-formylphenoxyacetic acid (201.3 mg, 0.703 mmol, 1.1 equiv), EDCI (183.8 mg, 0.959 mmol, 1.5 equiv). After stirring for 2 h at room temperature, the resulting mixture was diluted with water and extracted with EA (3×30 mL). The combined organic layers were washed with brine (50 mL), dried over anhydrous Na₂SO₄. After filtration, the filtrate was concentrated under reduced pressure. The crude product was purified by silica gel column chromatography, eluted with MeOH/DCM (1:10) to afford (1R,3S)-3-(5-{2-[3-(benzyloxy)-2-formylphenoxy]acetamido}-2H-pyrazol-3-yl)cyclopentyl N-cyclopropylcarbamate (210.0 mg, 41.57%) as a light yellow solid. MS (ESI) calcd. for C₂₈H₃₀N₄O₆, 518.22 m/z, found 519.15 [M+H]⁺.

### Step 4: Synthesis of (1R,3S)-3-(3-(2-(2-formyl-3-hydroxyphenoxy)acetamido)-1H-pyrazol-5-yl)cyclopentyl cyclopropylcarbamate; trifluoroacetic acid

To a solution of (1R,3S)-3-(5-{2-[3-(benzyloxy)-2-(1,3-dioxolan-2-yl)phenoxy]acetamido}-2H-pyrazol-3-yl)cyclopentyl N-cyclopropylcarbamate (100.0 mg, 0.178 mmol, 1 equiv) in EA (1.5 mL)/THF (1.5 mL) was added 10% Pd/C (94.5 mg, 0.089 mmol, 0.5 equiv). After stirring for 2 h at room temperature under H₂ atmosphere, desired product was detected by LCMS. After filtration, the filtrate was concentrated under reduced pressure. The crude product was purified by Prep-HPLC with the following conditions: Column: X Select CSH Fluoro Phenyl 30×150 mm, 5 μm; Mobile Phase A: Water (0.05% TFA), Mobile Phase B: ACN; Flow rate: 60 mL/min; Gradient: 19% B to 39% B in 10 min; Wave Length: 254/220 nm; RT1 (min): 8.6 to (1R,3S)-3-{5-[2-(2-formyl-3-hydroxyphenoxy)acetamido]-2H-pyrazol-3-yl}cyclopentyl N-cyclopropylcarbamate; trifluoroacetic acid (6.1 mg, 6.27%) as a white solid. MS (ESI) calcd. for C₂₁H₂₄N₄O₆, 428.17 m/z, found 429.10 [M+H]⁺. ¹H NMR (300 MHz, DMSO-d₆) δ (ppm): 11.71 (s, 1H), 10.52 (s, 1H), 10.39 (s, 1H), 7.51 (t, J=8.4 Hz, 1H), 7.23-7.26 (m, 1H), 6.55 (m, 2H), 6.31 (s, 1H), 4.98-5.00 (m, 1H), 4.82 (s, 2H), 3.02-3.08 (m, 1H), 2.41-2.46 (m, 2H), 1.81-2.02 (m, 2H), 1.58-1.72 (m, 3H), 0.50-0.56 (m, 2H), 0.34-0.39 (m, 2H). ¹⁹F NMR (282 MHz, DMSO-d₆) δ (ppm): −74.44.

### Example 4.21: Synthesis of Compound 87: (1R,3S)-3-(3-(2-(2-formyl-3-hydroxyphenoxy) acetamido)-1H-pyrazol-5-yl)cyclopentyl cyclobutylcarbamate

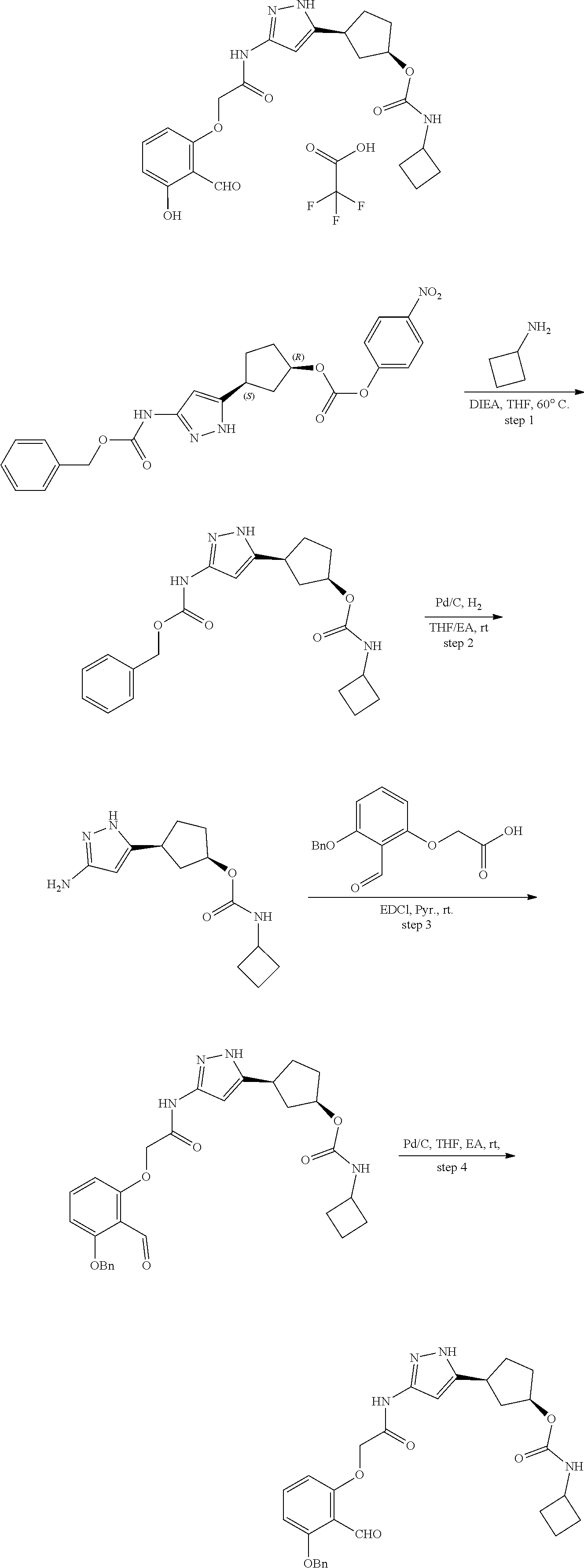

### Step 1: Synthesis of (1R,3S)-3-(3-(((benzyloxy)carbonyl)amino)-1H-pyrazol-5-yl)cyclopentyl cyclobutylcarbamate

To a stirred solution of (1R,3S)-3-(5-{[(benzyloxy)carbonyl]amino}-2H-pyrazol-3-yl)cyclopentyl 4-nitrophenyl carbonate (1.0 g, 2.144 mmol, 1 equiv) in THF (15 mL) was added cyclobutylamine (0.46 g, 6.432 mmol, 3 equiv) and DIEA (1.39 g, 10.720 mmol, 5 equiv) at room temperature. The resulting mixture was stirred for 2 h. The reaction was monitored by LCMS. The reaction was quenched by the solution of 0.5 M NaOH (50 mL) at room temperature. The resulting mixture was extracted with EA (3×50 mL). The combined organic layers were washed with 0.5 M NaOH (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O, 10% to 95% gradient in 30 min; detector, UV 220 nm. This resulted in (1R,3S)-3-(5-{[(benzyloxy)carbonyl]amino}-2H-pyrazol-3-yl)cyclopentyl N-cyclobutyl carbamate (0.5 g, 58.53%) as a white solid. MS (ESI) calcd. for C₂₁H₂₆N₄O₄, 398.20 m/z, found 399.25 [M+H]⁺.

### Step 2: Synthesis of (1R,3S)-3-(3-amino-1H-pyrazol-5-yl)cyclopentyl cyclobutylcarbamate

To a stirred solution of (1R,3S)-3-(5-{[(benzyloxy)carbonyl]amino}-2H-pyrazol-3-yl)cyclopentyl N-cyclobutylcarbamate (0.45 g, 1.129 mmol, 1 equiv) in THF (4 mL) and EA (4 mL) was added 10% Pd/C (0.3 g, 0.282 mmol, 0.25 equiv) at room temperature under H₂ atmosphere. The resulting mixture was stirred for 2 h under and atmosphere of H₂. The reaction was monitored by LCMS. The resulting mixture was filtered, the filter cake was washed with EA (4×150 mL). The filtrate was concentrated under reduced pressure. This resulted in (1R,3S)-3-(5-amino-2H-pyrazol-3-yl)cyclopentyl N-cyclobutylcarbamate (0.25 g, 83.75%) as an off-white solid. MS (ESI) calcd. for C₁₃H₂₀N₄O₂, 264.16 m/z, found 265.15 [M+H]⁺.

### Step 3: Synthesis of (1R,3S)-3-(3-(2-(3-(benzyloxy)-2-formylphenoxy)acetamido)-1H-pyrazol-5-yl)cyclopentyl cyclobutylcarbamate

(1R,3S)-3-(5-amino-2H-pyrazol-3-yl)cyclopentyl N-cyclobutylcarbamate (0.12 g, 0.454 mmol, 1 equiv), 3-(benzyloxy)-2-formylphenoxyacetic acid (0.13 g, 0.454 mmol, 1 equiv) and EDCI (0.13 g, 0.681 mmol, 1.5 equiv), were suspended in pyridine and stirred for 10 h at room temperature. The resulting mixture was diluted with H₂O (10 mL) and extracted with EA (3×10 mL). The combined organic layers were dried over anhydrous Na₂SO₄. After filtration, the filtrate was concentrated under reduced pressure. The crude was then subjected to silica gel chromatography (0-95% EtOAc/PE) to give (1R,3S)-3-(5-{2-[3-(benzyloxy)-2-formylphenoxy]acetamido}-2H-pyrazol-3-yl)cyclopentyl N-cyclobutylcarbamate (0.7 g, 289.51%) as a light yellow solid. MS (ESI) calcd. for C₂₉H₃₂N₄O₆, 532.23 m/z, found 533.30 [M+H]⁺.

### Step 4: Synthesis of (1R,3S)-3-(3-(2-(2-formyl-3-hydroxyphenoxy)acetamido)-1H-pyrazol-5-yl)cyclopentyl cyclobutylcarbamate

To a solution of (1R,3S)-3-(5-{2-[3-(benzyloxy)-2-formylphenoxy]acetamido}-2H-pyrazol-3-yl)cyclopentyl N-cyclobutylcarbamate (60.0 mg, 0.113 mmol, 1 equiv) in THF (3 mL) and EA (3 mL) was added 10% Pd/C (60.0 mg, 0.056 mmol, 0.50 equiv). After stirring for 3 h at room temperature under hydrogen atmosphere, the reaction mixture was filtered through a Celite pad, the filter cake was washed with EA (3×3 mL). The filtrate was concentrated under reduced pressure. The residue was purified by HPLC (Column: Xselect CSH Prep C18 OBD Column, 30×150 mm, 5 um; Mobile Phase A: Water (0.05% TFA), Mobile Phase B: ACN; Flow rate: 60 mL/min; Gradient: 26% B to 48% B in 10 min; Wave Length: 254/220 nm) to afford desired product (1R,3S)-3-{5-[2-(2-formyl-3-hydroxyphenoxy)acetamido]-2H-pyrazol-3-yl}cyclopentyl N-cyclobutylcarbamate; trifluoroacetic acid (6.8 mg, 10.74%) as an off-white solid. MS (ESI) calcd. for C₂₂H₂₆N₄O₆, 442.19 m/z, found 443.20 [M+H]⁺. ¹H NMR (400 MHz, DMSO-d₆) δ 11.71 (s, 1H), 10.54 (s, 1H), 10.40 (s, 1H), 7.47-7.61 (m, 1H), 7.37 (d, J=8.0 Hz, 1H), 6.50-6.60 (m, 2H), 6.31 (s, 1H), 4.91-5.06 (m, 1H), 4.82 (s, 2H), 3.86-4.06 (m, 1H), 2.91-3.15 (m, 1H), 2.35-2.48 (m, 1H), 1.95-2.17 (m, 3H), 1.79-1.94 (m, 3H), 1.64-1.78 (m, 2H), 1.46-1.63 (m, 3H). ¹⁹F NMR (376 MHz, DMSO) δ −74.35.

### Example 4.22: Synthesis of Compound 88: (1R,3S)-3-(3-(2-(2-formyl-3-hydroxyphenoxy) acetamido)-1H-pyrazol-5-yl)cyclopentyl cyclopentylcarbamate

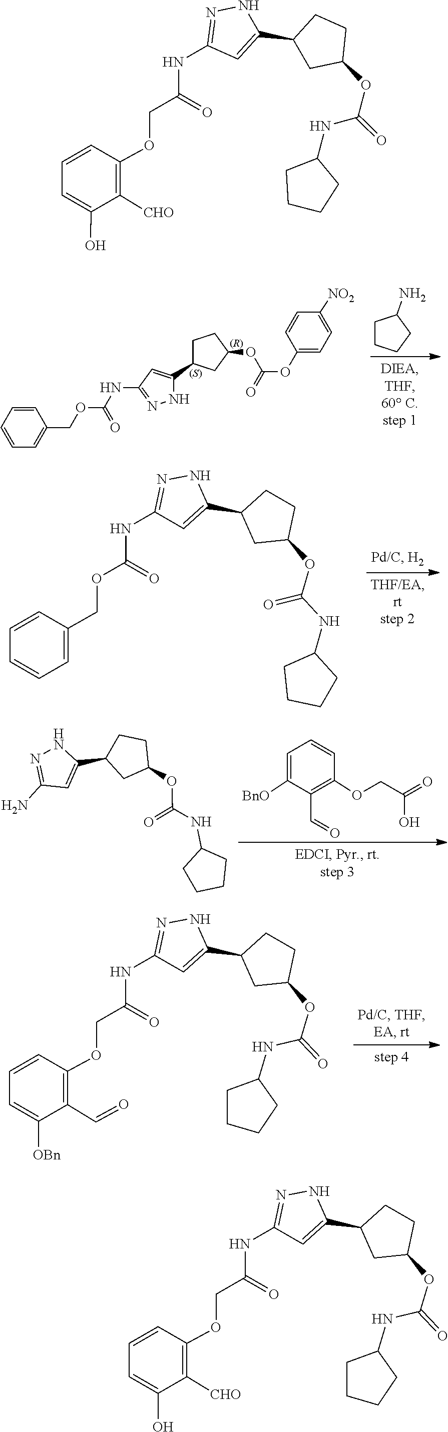

### Step 1: Synthesis of (1R,3S)-3-(5-{[(benzyloxy)carbonyl]amino}-2H-pyrazol-3-yl)cyclopentyl N-cyclopentylcarbamate

A solution of (1R,3S)-3-(5-{[(benzyloxy)carbonyl]amino}-2H-pyrazol-3-yl)cyclopentyl 4-nitrophenyl carbonate (1.2 g, 2.573 mmol, 1 equiv), cyclopentanamine (0.66 g, 7.719 mmol, 3 equiv) and DIEA (2 mL, 11.482 mmol, 4.46 equiv) in THF (10 mL) was stirred for 5 h at 60° C. The resulting mixture was cooled to room temperature, diluted with water and extracted with EA (3×30 mL). The combined organic layers were washed with saturated brine (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, The TFA aqueous solution of 0.05% in ACN, 10% to 50% gradient in 30 min; detector, UV 254 nm. to afford (1R,3S)-3-(5-{[(benzyloxy)carbonyl]amino}-2H-pyrazol-3-yl)cyclopentyl N-cyclopentylcarbamate (500.0 mg, 47.12%) as a white solid. MS (ESI) calcd. for C₂₂H₂₈N₄O₄, 412.49 m/z, found 413.20 [M+H]⁺

### Step 2: Synthesis of (1R,3S)-3-(5-amino-2H-pyrazol-3-yl)cyclopentyl N-cyclopentyl-carbamate

A solution of (1R,3S)-3-(5-{[(benzyloxy)carbonyl]amino}-2H-pyrazol-3-yl)cyclopentyl N-cyclopentylcarbamate (460 mg, 1.115 mmol, 1 equiv) and 10% Pd/C (460.47 mg) in EA (5 mL) and THF (5 mL) was stirred under H₂ for 3 h at rt. Desired product could be detected by LCMS. The resulting mixture was filtered, the filter cake was washed with EA (3×10 mL). The filtrate was concentrated under reduced pressure. The crude product (1R,3S)-3-(5-amino-2H-pyrazol-3-yl)cyclopentyl N-cyclopentylcarbamate (280 mg, 90.20%) was used in the next step directly without further purification. MS (ESI) calcd. for C₁₄H₂₂N₄O₂, 278.35 m/z, found 279.15 [M+H]⁺.

### Step 3: Synthesis of (1R,3S)-3-(5-{2-[3-(benzyloxy)-2-formylphenoxy]acetamido}-2H-pyrazol-3-yl)cyclopentyl N-cyclopentylcarbamat

A solution of (1R,3S)-3-(5-amino-2H-pyrazol-3-yl)cyclopentyl N-cyclopentylcarbamate (140.0 mg, 0.503 mmol, 1 equiv) and 3-(benzyloxy)-2-formylphenoxyacetic acid (143.99 mg, 0.503 mmol, 1 equiv) and EDCI (144.62 mg, 0.754 mmol, 1.5 equiv) in pyridine (5 mL) was stirred for 5 h at r.t. The resulting mixture was diluted with water and extracted with EA (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE:EA (0%-100%) to afford (1R,3S)-3-(5-{2-[3-(benzyloxy)-2-formylphenoxy]acetamido}-2H-pyrazol-3-yl)cyclo pentyl N-cyclopentyl-carbamate (70.0 mg, 25.46%) as a yellow solid. MS (ESI) calcd. for C₃₀H₃₄N₄O₆, 546.25 m/z, found 546.90 [M+H]⁺.

### Step 4: Synthesis of (1R,3S)-3-{5-[2-(2-formyl-3-hydroxyphenoxy)acetamido]-2H-pyrazol-3-yl}cyclopentyl N-cyclopentylcarbamate

A solution of (1R,3S)-3-(5-{2-[3-(benzyloxy)-2-formylphenoxy]acetamido}-2H-pyrazol-3-yl)cyclopentyl N-cyclopentylcarbamate (20 mg, 0.037 mmol, 1 equiv) and 10% Pd/C (20 mg) in THF (2 mL) was stirred under H₂ for 3 h at rt. Desired product could be detected by LCMS. The resulting mixture was filtered, the filter cake was washed with EA (3×10 mL). The filtrate was concentrated under reduced pressure. The residue was purified by reversed-phase flash chromatography with the following conditions: column, C18 silica gel; mobile phase; H₂O/ACN with 0.05% TFA, 10% to 50% gradient in 30 min; detector, UV 254 nm. to afford (1R,3S)-3-{5-[2-(2-formyl-3-hydroxyphenoxy)acetamido]-2H-pyrazol-3-yl}cyclopentyl N-cyclopentyl carbamate (2.0 mg, 11.83%) as a white solid. MS (ESI) calcd. for C₂₃H₂₈N₄O₆, 456.20 m/z, found 457.30 [M+H]⁺. ¹H NMR (400 MHz, DMSO-d₆) δ (ppm): 10.37 (s, 1H), 7.46-7.51 (m, 1H), 6.49-6.56 (m, 2H), 6.28 (s, 1H), 4.91-4.99 (m, 1H), 4.80 (s, 2H), 3.75 (s, 1H), 3.01-3.14 (m, 1H), 2.50 (s, 1H), 2.01-2.03 (m, 1H), 1.82-1.96 (m, 1H), 1.64-1.81 (m, 4H), 1.51-1.63 (m, 3H), 1.40-1.50 (m, 2H), 1.22-1.39 (m, 2H). ¹⁹F NMR (376 MHz, DMSO-d₆) δ (ppm): −73.88.

### Example 4.23: Synthesis of Compound 95: (1R,3S)-3-{5-[2-(2-formyl-3-methoxyphenoxy)acetamido]-2H-pyrazol-3-yl}cyclopentyl N-isopropylcarbamate; trifluoroacetic acid

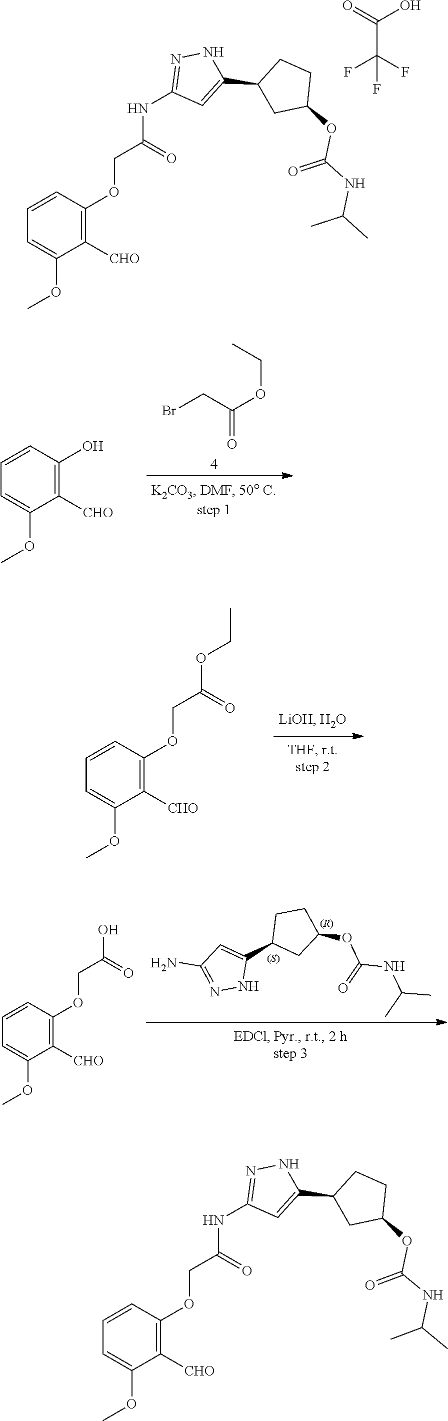

### Step 1: Synthesis of ethyl 2-(2-formyl-3-methoxyphenoxy)acetate

To a stirred solution of 2-hydroxy-6-methoxybenzaldehyde (2.5 g, 16.431 mmol, 1 equiv) and K₂CO₃ (4.54 g, 32.862 mmol, 2.00 equiv) in DMF (25 mL) was added ethyl bromoacetate (3.64 mL, 32.862 mmol, 2 equiv) in portions and let stir for 3 h at 50° C. The resulting mixture was cooled to room temperature, diluted with water and extracted with DCM (3×40 mL). The combined organic layers were washed with water (2×50 mL), and dried over anhydrous Na₂SO₄. After filtration, the filtrate was concentrated under reduced pressure to afford ethyl 2-(2-formyl-3-methoxyphenoxy)acetate (3.5 g, 83.72%) as a yellow solid. MS (ESI) calcd. for C₁₂H₁₄O₅, 238.08 m/z, found 239.00 [M+H]⁺

### Step 2: Synthesis of 2-(2-formyl-3-methoxyphenoxy)acetic acid

To a stirred solution of ethyl 2-(2-formyl-3-methoxyphenoxy)acetate (1.3 g, 5.457 mmol, 1 equiv) in tetrahydrofuran (7 mL, 27.285 mmol) and H₂O (7 mL) was added LiOH (0.65 g, 27.285 mmol, 5 equiv), the reaction was stirred at room temperature for 2 h. The mixture was neutralized to pH 6 with HCl (0.5 mol/L), extracted with EA (3×40 mL). The combined organic layers were washed with water (2×40 mL), dried over anhydrous Na₂SO₄. After filtration, the filtrate was concentrated under reduced pressure to afford 2-formyl-3-methoxyphenoxyacetic acid (900 mg, 76.24%) as a yellow solid. MS (ESI) calcd. for C₁₀H₁₀O₅, 210.05 m/z, found 211.05 [M+H]⁺.

### Step 3: Synthesis of (1R,3S)-3-{5-[2-(2-formyl-3-methoxyphenoxy)acetamido]-2H-pyrazol-3-yl}cyclopentyl N-isopropylcarbamate; trifluoroacetic acid

To a stirred solution of 2-formyl-3-methoxyphenoxyacetic acid (83.3 mg, 0.396 mmol, 1 equiv) and (1R,3S)-3-(5-amino-2H-pyrazol-3-yl)cyclopentyl N-isopropylcarbamate (100 mg, 0.396 mmol, 1.00 equiv) in pyridine (4 mL) was added EDCI (113.9 mg, 0.594 mmol, 1.5 equiv) in portions at room temperature and let stir for 2 h at room temperature. The reaction was monitored by LCMS. The resulting mixture was diluted with water and extracted with EtOAc (3×20 mL). The combined organic layers were washed with brine (2×40 mL), dried over anhydrous Na₂SO₄. After filtration, the filtrate was concentrated under reduced pressure. The crude product was purified by Prep-HPLC with the following conditions (Column: Xselect CSH Prep C18 OBD Column, 30×150 mm, 5 um; Mobile Phase A: water (0.05% TFA), Mobile Phase B: ACN; Flow rate: 60 mL/min; Gradient: 26% B to 48% B in 10 min; Wave Length: 254/220 nm) to afford (1R,3S)-3-{5-[2-(2-formyl-3-methoxyphenoxy)acetamido]-2H-pyrazol-3-yl}cyclopentyl N-isopropylcarbamate; trifluoroacetic acid (10.1 mg, 4.54%) as a white solid. MS (ESI) calcd. For C₂₄H₂₉F₃N₄O₈, 444.20 m/z, found 445.15 [M+H]⁺. 1H NMR (400 MHz, DMSO-d₆) δ 10.44 (s, 1H), 10.32 (s, 1H), 7.58 (t, J=8.6 Hz, 1H), 6.95-6.97 (m, 1H), 6.82 (d, J=8.8 Hz, 1H), 6.71 (d, J=6.2 Hz, 1H), 6.34 (s, 1H), 5.00 (s, 1H), 4.76 (s, 2H), 3.88 (s, 3H), 3.54-3.61 (m, 1H), 3.04-3.08 (m, 1H), 2.44-2.47 (m, 1H), 1.98-2.02 (m, 1H), 1.85-1.93 (m, 1H), 1.66-1.72 (m, 2H), 1.59-1.62 (m, 1H), 1.02 (d, J=6.8 Hz, 6H). ¹⁹F NMR (376 MHz, DMSO-d₆) δ −74.59.

### Example 4.24: Synthesis of Compound 97: (1R,3S)-3-{5-[2-(3-chloro-2-formylphenoxy)acetamido]-2H-pyrazol-3-yl}cyclopentyl N-isopropylcarbamate

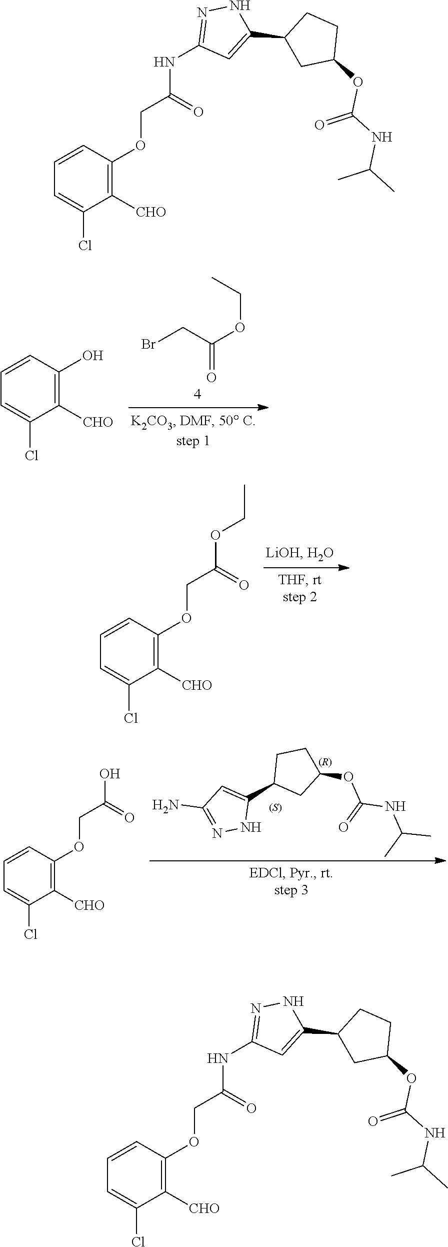

### Step 1: Synthesis of ethyl 2-(3-chloro-2-formylphenoxy)acetate

A solution of 2-chloro-6-hydroxybenzaldehyde (3.0 g, 19.161 mmol, 1 equiv) and ethyl bromoacetate (6.40 g, 38.322 mmol, 2 equiv) and K₂CO₃ (5.30 g, 38.322 mmol, 2 equiv) in DMF (30 mL) was stirred for 5 h at 50° C. The resulting mixture was cooled to room temperature, diluted with water and extracted with EA (30×3 mL). The combined organic layers were washed with Saturated NaCl solution (30×3 mL), dried over anhydrous Na₂SO₄. After filtration, the filtrate was concentrated under reduced pressure. The residue was purified by silica gel column chromatography, eluted with PE:EA (0-100%) to afford ethyl 2-(3-chloro-2-formylphenoxy) acetate (4.0 g, 86.03%) as a yellow solid. MS (ESI) calcd. for C₁₁H₁₁CO₄, 242.03 m/z, found 242.95 [M+H]⁺

### Step 2: Synthesis of 3-chloro-2-formylphenoxyacetic acid

A solution of ethyl 2-(3-chloro-2-formylphenoxy)acetate (1.0 g, 4.121 mmol, 1 equiv) and LiOH (0.25 g, 10.303 mmol, 2.5 equiv) in THF (10 mL) and H₂O (10 mL) was stirred for 2 h at rt. The reaction was monitored by LCMS. The resulting mixture was diluted with water and extracted with EA (30×3 mL). The combined organic layers were washed with saturated NaCl solution (30×3 mL), dried over anhydrous Na₂SO₄. After filtration, the filtrate was concentrated under reduced pressure. The residue was purified by silica gel column chromatography, eluted with PE:EA (0-100%) to afford 3-chloro-2-formylphenoxyacetic acid (500.0 mg, 56.54%) as a yellow solid. MS (ESI) calcd. for C₁₁H₁₁CO₄, 214.00 m/z, found 213.00 [M−H]⁻

### Step 3: Synthesis of (1R,3S)-3-{5-[2-(3-chloro-2-formylphenoxy)acetamido]-2H-pyrazol-3-yl}cyclopentyl N-isopropylcarbamate

A solution of 3-chloro-2-formylphenoxyacetic acid (120.0 mg, 0.559 mmol, 1 equiv) in pyridine (4 mL) treated with (1R,3S)-3-(5-amino-2H-pyrazol-3-yl)cyclopentyl N-isopropylcarbamate (120.0 mg, 0.476 mmol, 0.85 equiv) and EDCI (160.79 mg, 0.839 mmol, 1.5 equiv) was stirred for 2 h at rt. The resulting mixture was diluted with water and extracted with EA (30×3 mL). The combined organic layers were washed with saturated NaCl solution (30×3 mL), dried over anhydrous Na₂SO₄. After filtration, the filtrate was concentrated under reduced pressure. The residue was purified by reversed-phase flash chromatography with the following conditions: column, C18 silica gel; mobile phase; H₂O/ACN (with 0.05% TFA modifier), 10% to 50% gradient in 30 min; detector, UV 254 nm. to afford (1R,3S)-3-{5-[2-(3-chloro-2-formylphenoxy)acetamido]-2H-pyrazol-3-yl}cyclopentyl N-isopropylcarbamate (8.5 mg, 3.22%) as a white solid. MS (ESI) calcd. for C11H11CO4, 448.15 m/z, found 449.20 [M+H]+. 1H NMR (400 MHz, DMSO-d6) δ 10.47 (s, 1H), 10.46 (s, 1H), 7.57-7.61 (m, 1H), 7.13-7.20 (m, 2H), 6.94-6.96 (m, 1H), 6.32 (s, 1H), 4.92-5.01 (m, 1H), 4.87 (s, 2H), 3.54-3.57 (m, 1H), 3.03-3.07 (m, 1H), 2.45-2.46 (m, 1H), 1.98-2.01 (m, 1H), 1.90-1.99 (m, 1H), 1.57-1.72 (m, 3H), 1.03 (d, J=8 Hz, 6H). 19F NMR (376 MHz, DMSO-d6) δ (ppm): −74.70.

### Example 4.25: Synthesis of Compound 98: (1R,3S)-3-(3-(2-(2-formyl-3-(thiazol-2-yl)phenoxy) acetamido)-1H-pyrazol-5-yl)cyclopentyl isopropylcarbamate

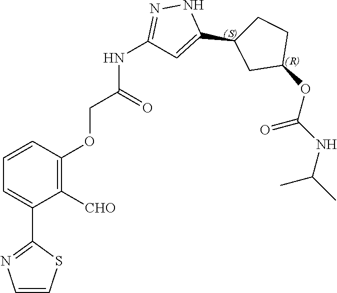

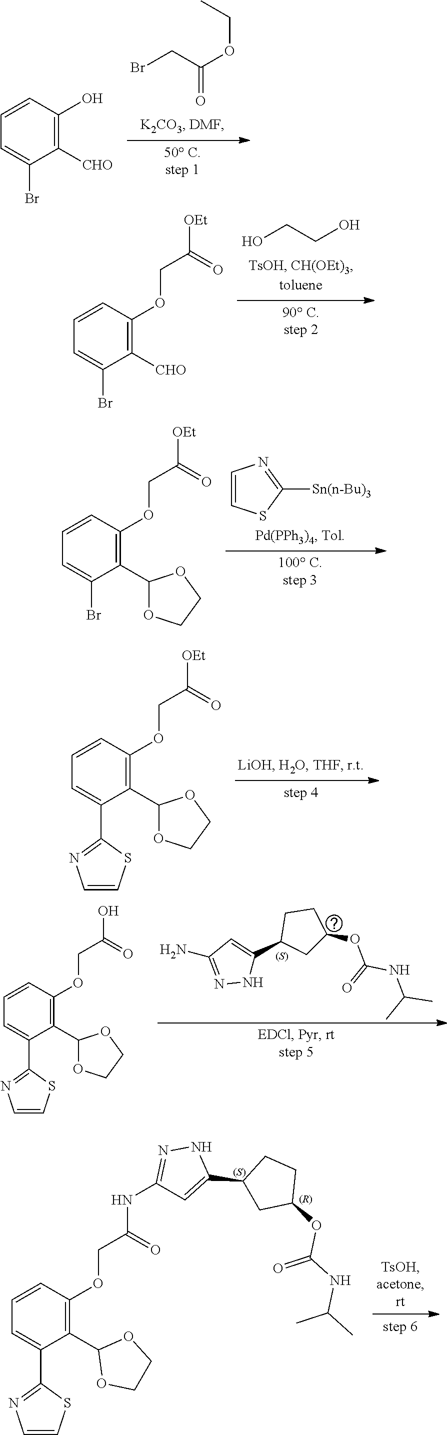

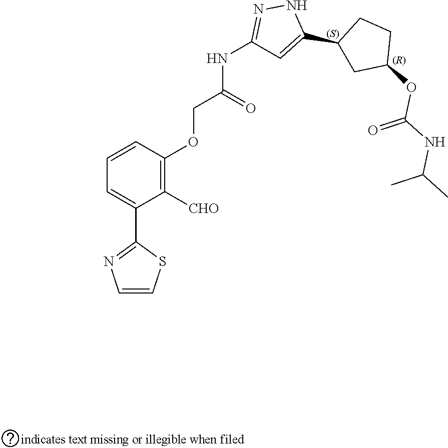

### Step 1: Synthesis of ethyl 2-(3-bromo-2-formylphenoxy)acetate

A solution of 2-bromo-6-hydroxybenzaldehyde (5 g, 24.873 mmol, 1 equiv), ethyl bromoacetate (4.98 g, 29.820 mmol, 1.20 equiv) and K₂CO₃ (10.29 g, 74.455 mmol, 2.99 equiv) in DMF (40 mL) was stirred for 2 h at 50° C. under N₂ atmosphere. The resulting mixture was cooled to room temperature, diluted with water and extracted with DCM (3×300 mL). The combined organic layers were washed with saturated NaCl (3×300 mL), dried over anhydrous Na₂SO₄. After filtration, the filtrate was concentrated under reduced pressure. This resulted in ethyl 2-(3-bromo-2-formylphenoxy)acetate (6.8 g, 81.68%) as an off-white solid. MS (ESI) calcd. for C₁₁H₁₁BrO₄, 285.98 m/z, found 286.95 [M+H]+.

### Step 2: Synthesis of ethyl 2-(3-bromo-2-(1,3-dioxolan-2-yl)phenoxy)acetate

A solution of ethyl 2-(3-bromo-2-formylphenoxy)acetate (3 g, 10.449 mmol, 1 equiv), ethylene glycol (2.9 g, 46.723 mmol, 4.47 equiv), p-toluenesulfonic acid (0.18 g, 1.045 mmol, 0.10 equiv) and (diethoxymethoxy)ethane (5.2 mL) in toluene (50 mL) was stirred for 2 h at 90° C. under N₂ atmosphere. Desired product could be detected by LCMS. The resulting mixture was cooled to room temperature, diluted with water, and extracted with DCM (3×200 mL). The combined organic layers were washed with saturated NaHCO₃ (3×200 mL), dried over anhydrous Na₂SO₄. After filtration, the filtrate was concentrated under reduced pressure. The residue was purified by silica gel column chromatography, eluted with PE/EA (0-34%) to afford ethyl 2-[3-bromo-2-(1,3-dioxolan-2-yl)phenoxy]acetate (2.5 g, 69.02%) as a yellow oil. MS (ESI) calcd. for C₁₃H₁₅BrO₅, 330.01 m/z, found 331.00 [M+H]+.

### Step 3: Synthesis of ethyl 2-(2-(1,3-dioxolan-2-yl)-3-(thiazol-2-yl)phenoxy)acetate

A mixture of ethyl 2-[3-bromo-2-(1,3-dioxolan-2-yl)phenoxy]acetate (700 mg, 2.114 mmol, 1 equiv), 2-(tributylstannyl)-1,3-thiazole (1194 mg, 3.191 mmol, 1.51 equiv) and tetrakis(triphenyl-phosphine)palladium(0) (366 mg, 0.317 mmol, 0.15 equiv) in toluene (15 mL) was stirred for 2 h at 100° C. under N₂ atmosphere. Desired product could be detected by LCMS. The resulting mixture was cooled to room temperature, diluted with water, and extracted with EA (3×100 mL). The combined organic layers were washed with saturated NaCl (3×100 mL), dried over anhydrous Na₂SO₄. After filtration, the filtrate was concentrated under reduced pressure. The residue was purified by silica gel column chromatography, eluted with PE/EA (0-90%) to afford ethyl 2-[2-(1,3-dioxolan-2-yl)-3-(1,3-thiazol-2-yl)phenoxy]acetate (600 mg, 82.37%) as a yellow oil. MS (ESI) calcd. for C₁₆H₁₇NO₅S, 335.08 m/z, found 336.10 [M+H]+.

### Step 4: Synthesis of 2-(2-(1,3-dioxolan-2-yl)-3-(thiazol-2-yl)phenoxy)acetic acid

A solution of ethyl 2-[2-(1,3-dioxolan-2-yl)-3-(1,3-thiazol-2-yl)phenoxy]acetate (500 mg, 1.491 mmol, 1 equiv) and lithium hydroxide (179 mg, 7.474 mmol, 5.01 equiv) in THF (2.5 mL)/water (2.5 mL) was stirred for 1 h at rt. The mixture was neutralized to pH 6 with citric acid. The resulting mixture was extracted with DCM (3×100 mL), dried over anhydrous Na₂SO₄. After filtration, the filtrate was concentrated under reduced pressure. This resulted in 2-(1,3-dioxolan-2-yl)-3-(1,3-thiazol-2-yl)phenoxyacetic acid (600, 84.73 a yellow solid. MS (ESI) calcd. for C₁₄H₁₃NO₅S, 307.05 m/z, found 308.05 [M+H]+.

### Step 5: Synthesis of (1R,3S)-3-(3-(2-(2-(1,3-dioxolan-2-yl)-3-(thiazol-2-yl)phenoxy) acetamido)-1H-pyrazol-5-yl)cyclopentyl isopropylcarbamate

A solution of 2-(1,3-dioxolan-2-yl)-3-(1,3-thiazol-2-yl)phenoxyacetic acid (146 mg, 0.475 mmol, 1 equiv), (1R,3S)-3-(5-amino-2H-pyrazol-3-yl)cyclopentyl N-isopropylcarbamate (120 mg, 0.475 mmol, 1 equiv) and EDCI (137 mg, 0.95 mmol, 2 equiv) in pyridine (5 mL) was stirred for 2 h at rt. Desired product could be detected by LCMS. The resulting mixture was diluted with water and extracted with DCM (3×100 mL). The combined organic layers were washed with saturated NaCl (3×100 mL), dried over anhydrous Na₂SO₄. After filtration, the filtrate was concentrated under reduced pressure. The crude product was purified by reverse phase flash with the following conditions (0-45% H₂O/ACN) to afford (1R,3S)-3-(5-{2-[2-(1,3-dioxolan-2-yl)-3-(1,3-thiazol-2-yl)phenoxy]acetamido}-2H-pyrazol-3-yl)cyclopentyl N-isopropylcarbamate (80 mg, 19.22%) as a yellow oil. MS (ESI) calcd. for C₂₆H₃₁N₅O₆S, 541.20 m/z, found 542.30 [M+H]⁺.

### Step 6: Synthesis of (1R,3S)-3-(3-(2-(2-formyl-3-(thiazol-2-yl)phenoxy)acetamido)-1H-pyrazol-5-yl)cyclopentyl isopropylcarbamate

(1R,3S)-3-(5-{2-[2-(1,3-dioxolan-2-yl)-3-(1,3-thiazol-2-yl)phenoxy]acetamido}-2H-pyrazol-3-yl)cyclopentyl N-isopropylcarbamate (60.0 mg, 0.111 mmol, 1 equiv) and TsOH (4.0 mg, 0.023 mmol, 0.21 equiv) was suspended in acetone (2 mL) and stirred for 2 h at rt. Desired product could be detected by LCMS. The resulting mixture was concentrated under reduced pressure. The crude product was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 34% B to 54% B in 10 min to afford (1R,3S)-3-(3-(2-(2-formyl-3-(thiazol-2-yl)phenoxy)acetamido)-1H-pyrazol-5-yl)cyclopentyl isopropylcarbamate (5.2 mg, 9.01%) as an off-white solid. MS (ESI) calcd. for C₂₄H₂₇N₅O₅S, 497.17 m/z, found 498.25 [M+H]+. ¹H NMR (400 MHz, DMSO-d₆) δ 12.18 (s, 1H), 10.24-10.29 (m, 2H), 7.94-8.01 (m, 2H), 7.65-7.69 (m, 1H), 7.42-7.44 (m, 1H), 7.29-7.31 (m, 1H), 6.95-6.97 (m, 1H), 6.34 (s, 1H), 4.95-5.02 (m, 1H), 4.85 (s, 2H), 3.53-3.60 (m, 1H), 3.04-3.08 (m, 1H), 2.44-2.48 (m, 1H), 1.99-2.02 (m, 1H), 1.84-1.94 (m, 1H), 1.71-1.73 (m, 2H), 1.65-1.69 (m, 1H), 0.95-1.05 (m, 6H).

### Example 4.26: Synthesis of Compound 99: (1R,3S)-3-(3-(2-(5-ethynyl-2-formyl-3-hydroxyphenoxy)acetamido)-1H-pyrazol-5-yl)cyclopentyl isopropylcarbamate

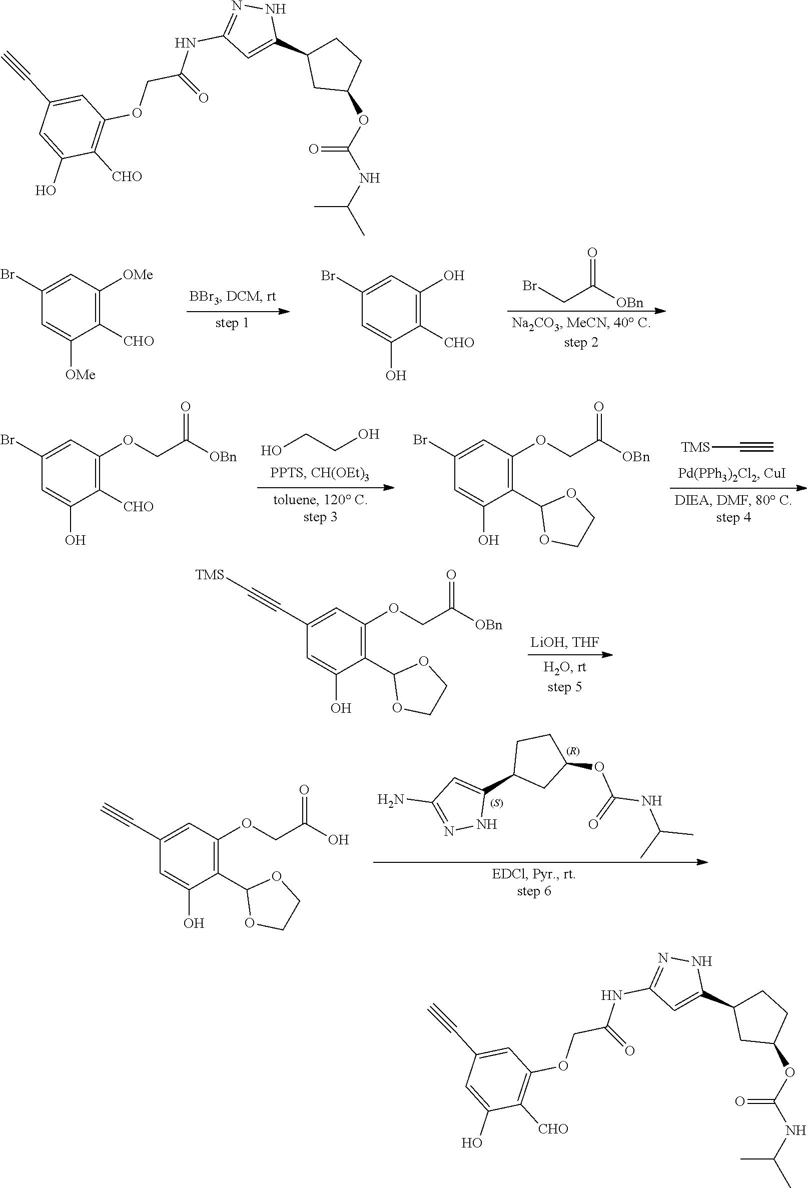

### Step 1: Synthesis of 4-bromo-2,6-dihydroxybenzaldehyde

To a stirred solution of 4-bromo-2,6-dimethoxybenzaldehyde (4.0 g, 16.322 mmol, 1 equiv) in DCM (50 mL) were added BBr₃ (81.61 mL, 81.610 mmol, 5 equiv) dropwise at 0° C. Then the mixture was warmed to room temperature and stirred for 2 h at room temperature. The resulting mixture was quenched with water dropwise at 0° C., then extracted with EA (100×3 mL). The combined organic layers were washed with Saturated NaCl solution (50×3 mL), dried over anhydrous Na₂SO₄. After filtration, the filtrate was concentrated under reduced pressure. The residue was purified by silica gel column chromatography, eluted with PE:EA (0-100%) to afford 4-bromo-2,6-dihydroxybenzaldehyde (2.2 g, 62.11%) as a white solid. MS (ESI) calcd. for C₇H₅BrO₃, 215.94 m/z, found 214.90 [M−H]⁻.

### Step 2: Synthesis of benzyl 2-(5-bromo-2-formyl-3-hydroxyphenoxy)acetate

A solution of 4-bromo-2,6-dihydroxybenzaldehyde (2.0 g, 9.216 mmol, 1 equiv) in ACN (2 mL) was treated with benzyl 2-bromoacetate (1055.55 mg, 4.608 mmol, 0.5 equiv) and Na₂CO₃ (2441.92 mg, 23.040 mmol, 2.5 equiv) were stirred for 2 h at 40° C. The resulting mixture was cooled to room temperature, diluted with water and extracted with EA (30 mL×3). The combined organic layers were washed with Saturated NaCl solution (30 mL×3), dried over anhydrous Na₂SO₄. After filtration, the filtrate was concentrated under reduced pressure. The residue was purified by silica gel column chromatography, eluted with PE:EA (0-100%) to afford benzyl 2-(5-bromo-2-formyl-3-hydroxyphenoxy)acetate (1.3 g, 38.63%) as a yellow solid. MS (ESI) calcd. for C₁₆H₁₃BrO₅, 363.99 m/z, found 362.90 [M−H]⁻.

### Step 3: Synthesis of benzyl 2-[5-bromo-2-(1,3-dioxolan-2-yl)-3-hydroxyphenoxy]acetate

To a stirred solution of benzyl 2-(5-bromo-2-formyl-3-hydroxyphenoxy)acetate (1.2 g, 3.286 mmol, 1 equiv) and PPTS (0.12 g, 0.493 mmol, 0.15 equiv) in toluene (15 mL) was added ethylene glycol (1.02 g, 16.430 mmol, 5 equiv), triethyl orthoformate (1.46 g, 9.858 mmol, 3 equiv) at room temperature, then the mixture was heated to 120° C. and stirred for 2 h. The resulting mixture was cooled to room temperature, diluted with water and extracted with EA (50×3 mL). The combined organic layers were washed with NaCl (50×3 mL), dried over anhydrous Na₂SO₄. The residue was purified by silica gel column chromatography, eluted with PE:EA to afford benzyl 2-[5-bromo-2-(1,3-dioxolan-2-yl)-3-hydroxyphenoxy]acetate (500.0 mg, 37.18%) as a yellow oil. MS (ESI) calcd. for C₁₈H₁₇BrO₆, 408.02 m/z, found 408.95 [M+H]⁺.

### Step 4: Synthesis of benzyl 2-[2-(1,3-dioxolan-2-yl)-3-hydroxy-5-[2-(trimethylsilyl)ethynyl]phenoxy]acetate

A solution of benzyl 2-[5-bromo-2-(1,3-dioxolan-2-yl)-3-hydroxyphenoxy]acetate (480.0 mg, 1.173 mmol, 1 equiv), trimethylsilylacetylene (576.03 mg, 5.865 mmol, 5 equiv), Pd(PPh₃)₂Cl₂ (123.49 mg, 0.176 mmol, 0.15 equiv), CuI (67.02 mg, 0.352 mmol, 0.3 equiv) and DIEA (454.79 mg, 3.519 mmol, 3 equiv) in DMF (5 mL) was stirred for 3 h at 80° C. under N₂ atmosphere. The resulting mixture was cooled to room temperature, diluted with water and extracted with EA (50×3 mL). The combined organic layers were washed with NaCl (50×3 mL), dried over anhydrous Na₂SO₄. The residue was purified by silica gel column chromatography, eluted with PE:EA to afford benzyl 2-[2-(1,3-dioxolan-2-yl)-3-hydroxy-5-[2-(trimethylsilyl)ethynyl]phenoxy]acetate (400.0 mg, 79.95%) as a yellow oil. MS (ESI) calcd. for C₂₃H₂₆O₆Si, 426.15 m/z, found 427.05 [M+H]⁺.

### Step 5: Synthesis of 5-ethynyl-2-formyl-3-hydroxyphenoxyacetic acid

A solution of benzyl 2-[2-(1,3-dioxolan-2-yl)-3-hydroxy-5-[2-(trimethylsilyl)ethynyl]phenoxy]acetate (350.0 mg, 0.821 mmol, 1 equiv) and LiOH (49.13 mg, 2.052 mmol, 2.5 equiv) in THF (3 mL) and H₂O (3 mL) was stirred for 2 h at r.t. The reaction was monitored by LCMS. The mixture was acidified to pH 5 with 1 mol/L HCl. The resulting mixture was extracted with EA (30×3 mL). The combined organic layers were washed with saturated NaCl solution (30×3 mL), dried over anhydrous Na₂SO₄. After filtration, the filtrate was concentrated under reduced pressure. The residue was purified by silica gel column chromatography, eluted with PE:EA (0-100%) to afford 5-ethynyl-2-formyl-3-hydroxyphenoxyacetic acid (70.0 mg, 38.74%) as a yellow solid. MS (ESI) calcd. for C₁₃H₁₂O₆, 220.04 m/z, found 219.00 [M−H]⁻.

### Step 6: Synthesis of (1R,3S)-3-{5-[2-(5-ethynyl-2-formyl-3-hydroxyphenoxy)acetamido]-2H-pyrazol-3-yl}cyclopentyl N-isopropylcarbamate; trifluoroacetic acid

A solution of 2-(1,3-dioxolan-2-yl)-5-ethynyl-3-hydroxyphenoxy acetic acid (50.0 mg, 0.189 mmol, 1 equiv) and (1R,3S)-3-(5-amino-2H-pyrazol-3-yl)cyclopentyl N-isopropylcarbamate (52.52 mg, 0.208 mmol, 1.1 equiv) and EDCI (90.69 mg, 0.473 mmol, 2.5 equiv) in pyridine (3 mL) was stirred for 5 h at r.t. Desired product could be detected by LCMS. The resulting mixture was diluted with water and extracted with EA (3×10 mL). The combined organic layers were washed with H₂O (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O/ACN with 0.05% TFA, 10% to 50% gradient in 30 min; detector, UV 254 nm. This resulted in (1R,3S)-3-{5-[2-(5-ethynyl-2-formyl-3-hydroxyphenoxy)acetamido]-2H-pyrazol-3-yl}cyclopentyl N-isopropylcarbamate; trifluoroacetic acid (4.4 mg, 4.02%) as a light yellow solid. MS (ESI) calcd. for C₂₃H₂₆N₄O₆, 454.19 m/z, found 455.20 [M+H]⁺. ¹H NMR (400 MHz, DMSO-d₆) δ (ppm): 11.68 (s, 1H), 10.52 (s, 1H), 10.35 (s, 1H), 6.95 (d, J=7.2 Hz, 1H), 6.66 (s, 1H), 6.62 (s, 1H), 6.31 (s, 1H), 4.99 (m, 1H), 4.85 (s, 2H), 4.57 (s, 1H), 3.54-3.59 (m, 1H), 3.03-3.07 (m, 1H), 2.43-2.49 (m, 1H), 1.97-2.02 (m, 1H), 1.83-1.93 (m, 1H), 1.54-1.73 (m, 3H), 1.02 (d, J=6.4 Hz, 6H). ¹⁹F NMR (376 MHz, DMSO-d₆) δ (ppm): −74.70.

### Example 4.27: Synthesis of Compound 101: (1R,3S)-3-{5-[2-(2-formyl-3-hydroxyphenoxy) acetamido]-2H-pyrazol-3-yl}cyclopentyl (2R)-2-methylpyrrolidine-1-carboxylate; trifluoro-acetic acid

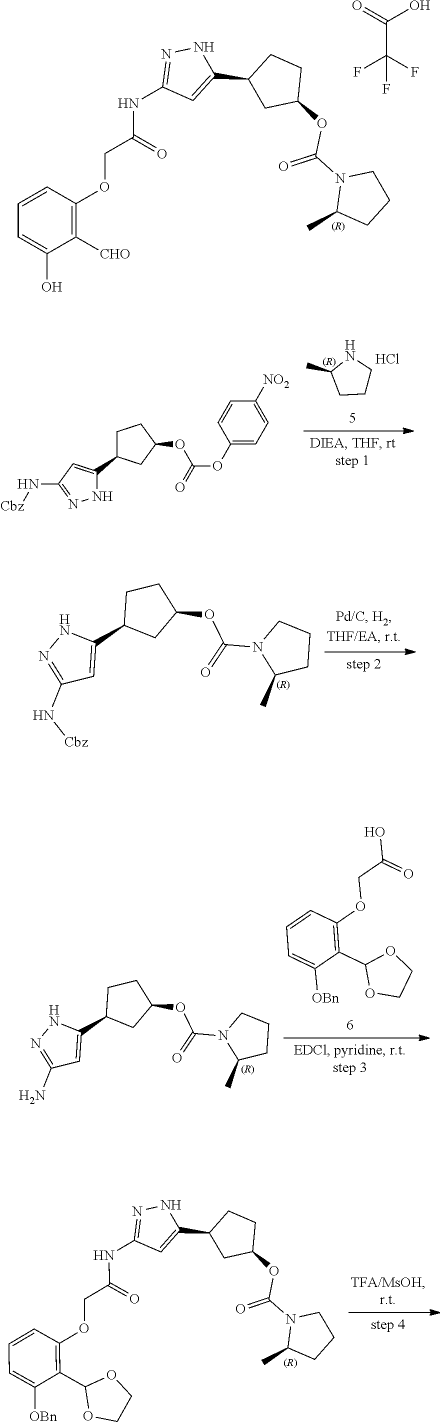

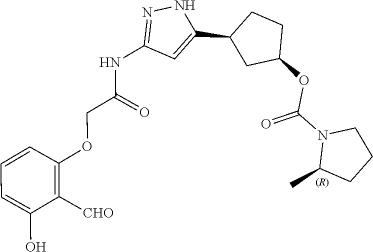

### Step 1: Synthesis of (1R,3S)-3-(5-{[(benzyloxy)carbonyl]amino}-2H-pyrazol-3-yl)cyclopentyl (2R)-2-methylpyrrolidine-1-carboxylate

To a stirred solution of (1R,3S)-3-(5-{[(benzyloxy)carbonyl]amino}-2H-pyrazol-3-yl)cyclopentyl 4-nitrophenyl carbonate (1 g, 2.144 mmol, 1 equiv) and (2R)-2-methylpyrrolidine hydrochloride (0.26 g, 2.144 mmol, 1 equiv) in THF (10 mL) was added DIEA (1.12 mL, 6.432 mmol, 3 equiv) and stirred for 2 h at room temperature. Then the resulting mixture was diluted with water and extracted with EtOAc (3×40 mL). The combined organic layers were washed with NaOH (0.5 mol/L, 2×40 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water, 5% to 70% gradient in 25 min; detector, UV 254 nm to afford (1R,3S)-3-(5-{[(benzyloxy)carbonyl]amino}-2H-pyrazol-3-yl)cyclopentyl (2R)-2-methylpyrrolidine-1-carboxylate (350 mg, 31.22%) as an off-white solid. MS (ESI) calcd. for C₂₂H₂₈N₄O₄, 412.21 m/z, found 413.25 [M+H]⁺

### Step 2: Synthesis of (1R,3S)-3-(5-amino-2H-pyrazol-3-yl)cyclopentyl (2R)-2-methylpyrrolidine-1-carboxylate

To a stirred solution of (1R,3S)-3-(5-{[(benzyloxy)carbonyl]amino}-2H-pyrazol-3-yl)cyclopentyl (2R)-2-methylpyrrolidine-1-carboxylate (350.0 mg, 0.849 mmol, 1 equiv) in THF (10 mL) and EA (10 mL) was added 10% Pd/C (300.0 mg, 2.819 mmol, 3.32 equiv) in portions and let stir for 2 h at room temperature under hydrogen atmosphere. The resulting mixture was filtered, the filter cake was washed with EA (4×30 mL). The filtrate was concentrated under reduced pressure to afford (1R,3S)-3-(5-amino-2H-pyrazol-3-yl)cyclopentyl (2R)-2-methylpyrrolidine-1-carboxylate (180 mg, 29.58%) as a yellow oil. MS (ESI) calcd. For C₁₄H₂₂N₄O₂, 278.17 m/z, found 279.20 [M+H]⁺.

### Step 3: Synthesis of (1R,3S)-3-(5-{2-[3-(benzyloxy)-2-(1,3-dioxolan-2-yl)phenoxy]acetamido}-2H-pyrazol-3-yl)cyclopentyl (2R)-2-methylpyrrolidine-1-carboxylate

To a stirred solution of (1R,3S)-3-(5-amino-2H-pyrazol-3-yl)cyclopentyl (2R)-2-methyl pyrrolidine-1-carboxylate (100.0 mg, 0.359 mmol, 1 equiv) and 3-(benzyloxy)-2-(1,3-dioxolan-2-yl)phenoxyacetic acid (118.6 mg, 0.359 mmol, 1 equiv) in pyridine (5 mL) was added EDCI (103.3 mg, 0.538 mmol, 1.5 equiv) in portions at room temperature overnight. Then the resulting mixture was diluted with water and extracted with EA (3×30 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O (0.05% mol/L NH₄HCO₃), 5% to 85% gradient in 20 min; detector, UV 254 nm. This resulted in (1R,3S)-3-(5-{2-[3-(benzyloxy)-2-(1,3-dioxolan-2-yl)phenoxy]acetamido}-2H-pyrazol-3-yl)cyclopentyl(2R)-2-methyl pyrrolidine-1-carboxylate (70.0 mg, 27.13%) as a light yellow solid. MS (ESI) calcd. For C₃₂H₃₈N₄O₇, 590.27 m/z, found 591.35 [M+H]⁺.

### Step 4: Synthesis of (1R,3S)-3-{5-[2-(2-formyl-3-hydroxyphenoxy)acetamido]-2H-pyrazol-3-yl}cyclopentyl (2R)-2-methylpyrrolidine-1-carboxylate; trifluoroacetic acid

To a stirred solution of (1R,3S)-3-(5-{2-[3-(benzyloxy)-2-(1,3-dioxolan-2-yl)phenoxy]acetamido}-2H-pyrazol-3-yl)cyclopentyl (2R)-2-methylpyrrolidine-1-carboxylate (60 mg, 0.102 mmol, 1 equiv) in methanesulfonic acid (2 mL) was added TFA (0.8 mL) in portions for 2 h at room temperature. The resulting mixture was concentrated under reduced pressure. The crude product was purified by Prep-HPLC with the following conditions (Column: SunFire Prep C18 OBD Column, 30×150 mm, 5 um; Mobile Phase A: water (0.05% TFA), Mobile Phase B: ACN; Flow rate: 60 mL/min; Gradient: 24% B to 46% B in 10 min; Wave Length: 254/220 nm) to afford (1R,3S)-3-{5-[2-(2-formyl-3-hydroxyphenoxy)acetamido]-2H-pyrazol-3-yl}cyclopentyl (2R)-2-methylpyrrolidine-1-carboxylate; trifluoroacetic acid (11.7 mg, 20.12%) as a white solid. MS (ESI) calcd. for C₂₅H₂₉F₃N₄O₈, 546.20 m/z, found 457.15 [M+H]⁺. ¹H NMR (400 MHz, DMSO-d₆) δ 11.72 (s, 1H), 10.54 (s, 1H), 10.40 (s, 1H), 7.51 (t, J=8.4 Hz, 1H), 6.52-6.57 (m, 2H), 6.33 (s, 1H), 5.02 (s, 1H), 4.83 (s, 2H), 3.77 (s, 1H), 3.24 (s, 2H), 3.09-3.13 (m, 1H), 2.41-2.51 (m, 1H), 2.02-2.03 (m, 1H), 1.72-1.91 (m, 7H), 1.45-1.46 (m, 1H), 1.06-1.08 (m, 3H). ¹⁹F NMR (376 MHz, DMSO-d₆) δ −74.77.

### Example 4.28: Synthesis of Compound 102: (S)-((1R,3S)-3-(3-(2-(2-formyl-3-hydroxyphenoxy) acetamido)-1H-pyrazol-5-yl)cyclopentyl) 2-methylpyrrolidine-1-carboxylate; trifluoroacetic acid

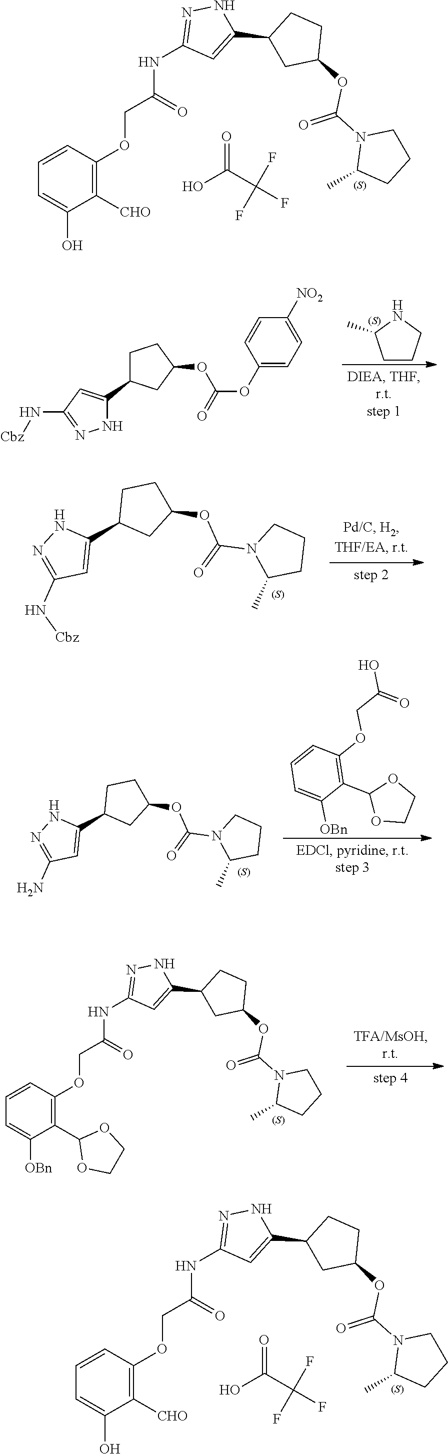

### Step 1: Synthesis of (S)-((1R,3S)-3-(3-(benzyloxycarbonylamino)-1H-pyrazol-5-yl)cyclo pentyl)-2-methylpyrrolidine-1-carboxylate

To a stirred solution of (1R,3S)-3-(5-{[(benzyloxy)carbonyl]amino}-2H-pyrazol-3-yl)cyclopentyl 4-nitrophenyl carbonate (1 g, 2.144 mmol, 1.00 equiv) and DIEA (831.3 mg, 6.432 mmol, 3 equiv) in THF (20 mL) was added (2S)-2-methylpyrrolidine (365.1 mg, 4.288 mmol, 2.00 equiv) in portions at room temperature. The resulting mixture was stirred for 2 h at room temperature. Then the resulting mixture was diluted with water and extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with CH₂Cl₂/MeOH (10:1) to afford (1R,3S)-3-(5-{[(benzyloxy)carbonyl]amino}-2H-pyrazol-3-yl)cyclopentyl (2S)-2-methylpyrrolidine-1-carboxylate (500.0 mg, 45.77%) as a white solid. MS (ESI) calcd. for C₂₂H₂₈N₄O₄, 412.21 m/z, found 413.15 [M+H]⁺.

### Step 2: Synthesis of (S)-((1R,3S)-3-(3-amino-1H-pyrazol-5-yl)cyclopentyl) 2-methyl pyrrolidine-1-carboxylate

To a stirred solution of (1R,3S)-3-(5-{[(benzyloxy)carbonyl]amino}-2H-pyrazol-3-yl)cyclopentyl (2S)-2-methylpyrrolidine-1-carboxylate (500.0 mg, 1.212 mmol, 1 equiv) in THF (10 mL) were added EA (20 mL) and 10% Pd/C (200.0 mg, 1.879 mmol, 1.55 equiv) at room temperature under hydrogen atmosphere. The resulting mixture was stirred for 4 h at room temperature under hydrogen atmosphere. The reaction was monitored by TLC (EA:PE=1:1). The resulting mixture was filtered, the filter cake was washed with EtOAc (3×30 mL). The filtrate was concentrated under reduced pressure to afford (1R,3S)-3-(5-amino-2H-pyrazol-3-yl)cyclopentyl (2S)-2-methylpyrrolidine-1-carboxylate (300.0 mg, 77.18%) as a light yellow oil.

### Step 3: Synthesis of (S)-((1R,3S)-3-(3-(2-(3-(benzyloxy)-2-(1,3-dioxolan-2-yl)phenoxy)acetamido)-1H-pyrazol-5-yl)cyclopentyl) 2-methylpyrrolidine-1-carboxylate

To a stirred solution of (1R,3S)-3-(5-amino-2H-pyrazol-3-yl)cyclopentyl (2S)-2-methylpyrrolidine-1-carboxylate (170.0 mg, 0.611 mmol, 1 equiv) and 3-(benzyloxy)-2-(1,3-dioxolan-2-yl)phenoxyacetic acid (201.8 mg, 0.611 mmol, 1.0 equiv) in pyridine (5 mL) was added EDCI (175.6 mg, 0.916 mmol, 1.5 equiv) at room temperature under air atmosphere. The resulting mixture was stirred for 16 h at room temperature. The reaction was monitored by TLC. Then the resulting mixture was diluted with water and extracted with EA (3×30 mL). The combined organic layers were washed with brine (2×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN/Water (10 mmol/L NH₄HCO₃), 5% to 90% gradient in 30 min; detector, UV 220 nm to afford (1R,3S)-3-(5-{2-[3-(benzyloxy)-2-(1,3-dioxolan-2-yl)phenoxy]acetamido}-2H-pyrazol-3-yl)cyclopentyl (2S)-2-methylpyrrolidine-1-carboxylate (60.0 mg, 16.63%) as a white solid.

### Step 4: Synthesis of (S)-((1R,3S)-3-(3-(2-(2-formyl-3-hydroxyphenoxy)acetamido)-1H-pyrazol-5-yl)cyclopentyl) 2-methylpyrrolidine-1-carboxylate; trifluoroacetic acid

To a stirred solution of (1R,3S)-3-(5-{2-[3-(benzyloxy)-2-(1,3-dioxolan-2-yl)phenoxy]acetamido}-2H-pyrazol-3-yl)cyclopentyl (2S)-2-methylpyrrolidine-1-carboxylate (60.0 mg, 0.102 mmol, 1 equiv) in trifluoroacetic acid (4.5 mL) was added methanesulfonic acid (1.5 mL) dropwise at room temperature under nitrogen atmosphere. The resulting mixture was stirred for 2 h at room temperature. The reaction was monitored by LCMS. The resulting mixture was concentrated under vacuum. The crude product was purified by Prep-HPLC with the following conditions (Column: Xselect CSH Prep C18 OBD Column, 30×150 mm, 5 um; Mobile Phase A: Water (0.1% FA), Mobile Phase B: ACN; Flow rate: 60 mL/min) to afford (S)-((1R,3S)-3-(3-(2-(2-formyl-3-hydroxyphenoxy)acetamido)-1H-pyrazol-5-yl)cyclopentyl) 2-methylpyrrolidine-1-carboxylate; trifluoroacetic acid (10.4 mg, 17.93%) as a white solid. MS (ESI) calcd. for C₂₃H₂₈N₄O₆, 456.20 m/z, found 457.15 [M+H]⁺. ¹H NMR (400 MHz, DMSO-d₆) δ 11.71 (s, 1H), 10.53 (s, 1H), 10.40 (s, 1H), 7.51 (t, J=8.4 Hz, 1H), 6.52-6.58 (m, 2H), 6.33 (s, 1H), 5.00-5.02 (m, 1H), 4.82 (s, 2H), 3.77-3.79 (m, 1H), 3.23-3.26 (m, 2H), 3.08-3.12 (m, 1H), 2.37-2.44 (m, 1H), 2.00-2.04 (m, 1H), 1.64-1.93 (m, 7H), 1.46-1.48 m, 1H), 1.03-1.10 (m, 3H). ¹⁹F NMR (376 MHz, DMSO-d₆) δ −74.83.

### Example 4.29: Synthesis of Compound 105: (1R,3S)-3-{5-[2-(2-formyl-3-methoxyphenoxy) acetamido]-2H-pyrazol-3-yl}cyclopentyl N-(1-methylcyclobutyl)carbamate; trifluoroacetic acid

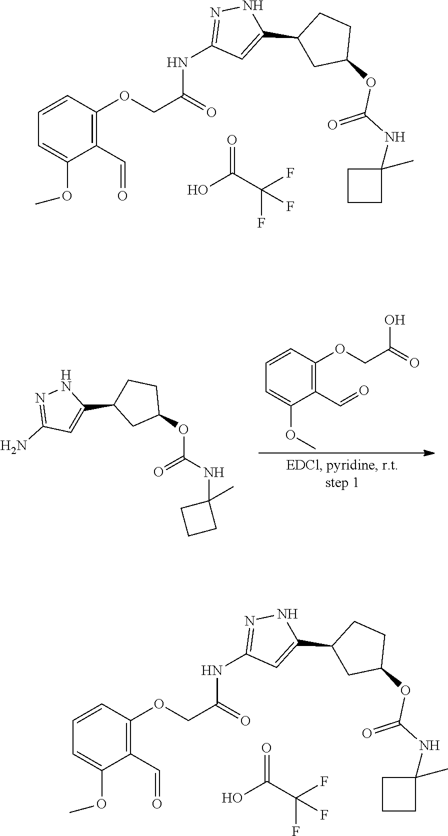

### Step 1: Synthesis of (1R,3S)-3-{5-[2-(2-formyl-3-methoxyphenoxy)acetamido]-2H-pyrazol-3-yl}cyclopentyl N-(1-methylcyclobutyl)carbamate; trifluoroacetic acid

To a stirred solution of 2-formyl-3-methoxyphenoxyacetic acid (135.9 mg, 0.647 mmol, 1 equiv) and (1R,3S)-3-(5-amino-2H-pyrazol-3-yl)cyclopentyl N-(1-methylcyclobutyl)carbamate (180.0 mg, 0.647 mmol, 1.00 equiv) in pyridine (10 mL) was added EDCI (186.0 mg, 0.971 mmol, 1.5 equiv) in portions at room temperature. The resulting mixture was stirred for 1 h at room temperature. The reaction was monitored by LCMS. The reaction mixture was then treated with H₂O (30 mL), dropwise over 10 min, extracted with EtOAc (30 mL×2), and the combined extracts washed with brine (30 mL), dried over anhydrous Na₂SO₄, filtered, and concentrated to dryness in vacuo to give a nearly yellow viscous oil. After filtration, the filtrate was concentrated under reduced pressure. The crude product was purified by Prep-HPLC with the following conditions (Column: XSelect CSH Prep C18 OBD Column 30×150 mm, 5 μm; Mobile Phase A: Water (0.05% TFA), Mobile Phase B: ACN; Flow rate: 60 mL/min; Gradient: 31% B to 52% B in 10 min; Wave Length: 254/220 nm; RT1 (min): 9.08) to afford (1R,3S)-3-{5-[2-(2-formyl-3-methoxyphenoxy)acetamido]-2H-pyrazol-3-yl}cyclopentyl N-(1-methylcyclobutyl)carbamate; trifluoroacetic acid (17.7 mg, 4.52%) as an off-white solid. MS (ESI) calcd. for C₂₄H₃₀N₄O₆, 470.22 m/z, found 471.15 [M+H]⁺. ¹H NMR (400 MHz, DMSO-d₆) δ 10.44 (s, 1H), 10.32 (s, 1H), 7.58 (t, J=8.4 Hz, 1H), 7.10-7.23 (m, 1H), 6.81-6.84 (m, 1H), 6.70-6.73 (m, 1H), 6.34 (s, 1H), 4.97-5.01 (m, 1H), 4.76 (s, 2H), 3.88 (s, 3H), 3.04-3.08 (m, 1H), 2.44-2.46 (m, 1H), 2.21-2.22 (m, 2H), 1.99-2.03 (m, 1H), 1.86-1.90 (m, 1H), 1.70-1.89 (m, 6H), 1.57-1.67 (m, 1H), 1.31 (s, 3H). ¹⁹F NMR (376 MHz, DMSO-d₆) δ −74.59.

### Example 4.30: Synthesis of Compound 27: (1R,3S)-3-{5-[5-(2-formyl-3-hydroxyphenoxymethyl)-2-methylpyrazole-3-amido]-2H-pyrazol-3-yl}cyclopentyl N-isopropylcarbamate

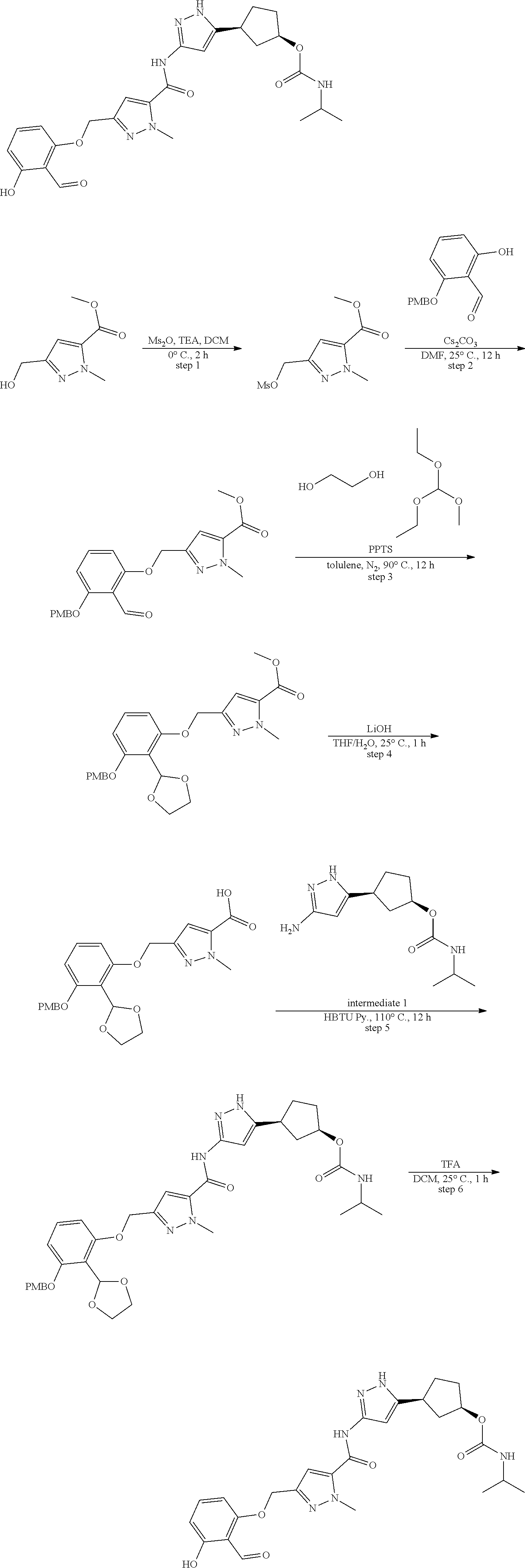

### Step 1: methyl 5-[(methanesulfonyloxy)methyl]-2-methylpyrazole-3-carboxylate

A solution of methyl 5-(hydroxymethyl)-2-methylpyrazole-3-carboxylate (700 mg, 4.114 mmol, 1 equiv) in DCM (10 mL) was treated with TEA (624.4 mg, 6.171 mmol, 1.5 equiv) followed by the addition of methanesulfonic anhydride (859.9 mg, 4.937 mmol, 1.2 equiv) in portions at 0° C. The resulting mixture was stirred for 2 h at 25° C. under N₂ atmosphere. The resulting mixture was diluted with DCM (50 mL) and washed with H₂O (30 mL×2), dried over anhydrous Na₂SO₄. After filtration, the filtrate was concentrated under reduced pressure. The residue was purified by silica gel column with PE/EA (0-70%) to afford methyl 5-[(methanesulfonyloxy)methyl]-2-methylpyrazole-3-carboxylate (450 mg, 41.86%) as a yellow oil. MS (ESI) calcd. for C₈H₁₂N₂O₅S, 248.05 m/z, found 249.35 [M+H]⁺.

### Step 2: methyl 5-{2-formyl-3-[(4-methoxyphenyl)methoxy]phenoxymethyl}-2-methylpyrazole-3-carboxylate

A solution of methyl 5-[(methanesulfonyloxy)methyl]-2-methylpyrazole-3-carboxylate (430 mg, 1.732 mmol, 1.3 equiv) in DMF (15 mL) was treated with Cs₂CO₃ (1.1 g, 3.331 mmol, 2.5 equiv) for 0.5 h at 25° C. followed by the addition of 2-hydroxy-6-[(4-methoxyphenyl)methoxy]benzaldehyde (344.1 mg, 1.332 mmol, 1.00 equiv) in portions at 25° C. The resulting mixture was stirred for 12 h at 25° C. under N₂ atmosphere. The resulting mixture was diluted with EA (50 mL) and washed with H₂O (30 mL×2), dried over anhydrous Na₂SO₄. After filtration, the filtrate was concentrated under reduced pressure. The residue was purified by silica gel column with PE/EA (0-70%) to afford methyl 5-{2-formyl-3-[(4-methoxyphenyl)methoxy]phenoxymethyl}-2-methylpyrazole-3-carboxylate (210 mg, 34.18%) as a white solid. MS (ESI) calcd. for C₂₂H₂₂N₂O₆, 410.15 m/z, found 411.15 [M+H]⁺.

### Step 3: methyl 5-[2-(1,3-dioxolan-2-yl)-3-[(4-methoxyphenyl)methoxy]phenoxymethyl]-2-methylpyrazole-3-carboxylate

A solution of methyl 5-{2-formyl-3-[(4-methoxyphenyl)methoxy]phenoxymethyl}-2-methylpyrazole-3-carboxylate (210 mg, 4.629 mmol, 1 equiv), ethylene glycol (1.7 g, 27.774 mmol, 6 equiv), PPTS (0.1 g, 0.463 mmol, 0.1 equiv) and triethyl orthoformate (2.1 g, 13.887 mmol, 3 equiv) in toluene (10 mL) was stirred at 90° C. for 12 h. The resulting mixture was diluted with EA (50 mL) and washed with H₂O (30 mL×2), dried over anhydrous Na₂SO₄. After filtration, the filtrate was concentrated under reduced pressure. The residue was purified by silica gel column with PE/EA (0-70%) to afford methyl 5-[2-(1,3-dioxolan-2-yl)-3-[(4-methoxy-phenyl)methoxy]phenoxymethyl]-2-methylpyrazole-3-carboxylate (210 mg, 8.98%) as a yellow solid. MS (ESI) calcd. for C₂₄H₂₆N₂O₇, 454.17 m/z, found 455.11 [M+H]⁺.

### Step 4: 5-[2-(1,3-dioxolan-2-yl)-3-[(4-methoxyphenyl)methoxy]phenoxymethyl]-2-methylpyrazole-3-carboxylic acid

A solution of methyl 5-[2-(1,3-dioxolan-2-yl)-3-[(4-methoxyphenyl)methoxy]phenoxymethyl]-2-methylpyrazole-3-carboxylate (200 mg, 0.440 mmol, 1 equiv) and LiOH H₂O (33.2 mg, 0.792 mmol, 1.8 equiv) in THF (5 mL) and H₂O (1 mL) was stirred at 25° C. for 1 h. The mixture was acidified to pH 7 with HCl (0.5 M). The resulting mixture was diluted with EA (50 mL) and washed with H₂O (30 mL×2), dried over anhydrous Na₂SO₄. After filtration, the filtrate was concentrated under reduced pressure to afford 5-[2-(1,3-dioxolan-2-yl)-3-[(4-methoxyphenyl)methoxy]phenoxymethyl]-2-methylpyrazole-3-carboxylic acid (86 mg, 40.82%) as a yellow solid. MS (ESI) calcd. for C₂₃H₂₄N₂O₇, 440.45 m/z, found 441.09 [M+H]⁺.

### Step 5: (1R,3S)-3-(5-{5-[2-(1,3-dioxolan-2-yl)-3-[(4-methoxyphenyl)methoxy]phenoxy-methyl]-2-methylpyrazole-3-amido}-2H-pyrazol-3-yl)cyclopentyl N-isopropylcarbamate

A solution of 5-[2-(1,3-dioxolan-2-yl)-3-[(4-methoxyphenyl)methoxy]phenoxymethyl]-2-methylpyrazole-3-carboxylic acid (86 mg, 0.307 mmol, 1.3 equiv), (1R,3S)-3-(5-amino-2H-pyrazol-3-yl)cyclopentyl N-isopropylcarbamate (59.5 mg, 0.236 mmol, 1 equiv) and HBTU (67.8 mg, 0.354 mmol, 1.5 equiv) in pyridine (5 mL) was stirred at 110° C. for 12 h. The resulting mixture was diluted with EA (50 mL) and washed with H₂O (30 mL×2), dried over anhydrous Na₂SO₄. After filtration, the filtrate was concentrated under reduced pressure. The residue was purified by Prep-TLC (MeOH/DCM 10%) to afford (1R,3S)-3-(5-{5-[2-(1,3-dioxolan-2-yl)-3-[(4-methoxyphenyl)methoxy]phenoxymethyl]-2-methylpyrazole-3-amido}-2H-pyrazol-3-yl)cyclopentyl N-isopropylcarbamate (30 mg, 17.16%) as a yellow solid. MS (ESI) calcd. for C₃₅H₄₂N₆O₈, 674.31 m/z, found 675.35 [M+H]⁺.

### Step 6: (1R,3S)-3-{5-[5-(2-formyl-3-hydroxyphenoxymethyl)-2-methylpyrazole-3-amido]-2H-pyrazol-3-yl}cyclopentyl N-isopropylcarbamate

A solution of (1R,3S)-3-(5-{5-[2-(1,3-dioxolan-2-yl)-3-[(4-methoxyphenyl)methoxy]phenoxy-methyl]-2-methylpyrazole-3-amido}-2H-pyrazol-3-yl)cyclopentyl N-isopropylcarbamate (30 mg, 0.044 mmol, 1 equiv) in DCM (0.5 mL, 7.865 mmol, 176.91 equiv) and TFA (0.1 mL, 1.346 mmol, 30.28 equiv) was stirred at 25° C. for 1 h. Then the crude product was further purified by Prep-HPLC with the following condition: Column; Xselect CSH C18 OBD Column, 30×150 mm, 5 μm; Mobile Phase A: Water (0.1% FA), Mobile Phase B: ACN; Flow rate: 60 mL/min; Gradient: 32% B to 62% B in 7 min; Wave Length: 254/220 nm; RT1 (min): 6.18, afforded (1R,3S)-3-{5-[5-(2-formyl-3-hydroxyphenoxymethyl)-2-methylpyrazole-3-amido]-2H-pyrazol-3-yl}cyclopentyl N-isopropylcarbamate (1.5 mg, 6.54%) as a white solid. MS (ESI) calcd. for C₂₅H₃₀N₆O₆, 510.22 m/z, found 511.25 [M+H]⁺. ¹H NMR (300 MHz, DMSO-d₆) δ (ppm): 12.07 (s, 1H), 11.76 (s, 1H), 10.78 (s, 1H), 10.35 (s, 1H), 7.56 (t, J=8.4 Hz, 1H), 7.31 (s, 1H), 6.95 (s, 1H), 6.78 (d, J=8.4 Hz, 1H), 6.56 (d, J=8.3 Hz, 1H), 6.43 (s, 1H), 5.21 (s, 2H), 5.01 (s, 1H), 4.10 (s, 3H), 3.59 (q, J=6.9 Hz, 1H), 3.09 (s, 1H), 2.03 (s, 1H), 1.91 (s, 1H), 1.79-1.70 (m, 2H), 1.62 (s, 1H), 1.24 (s, 1H), 1.04 (d, J=6.6 Hz, 6H).

### Example 4.31: Synthesis of Compound 77: (1R,3S)-3-(5-[5-(2-formyl-3-hydroxy-5-methoxyphenoxy methyl)-2-methylpyrazole-3-amido]-2H-pyrazol-3-ylcyclopentyl N-isopropylcarbamate

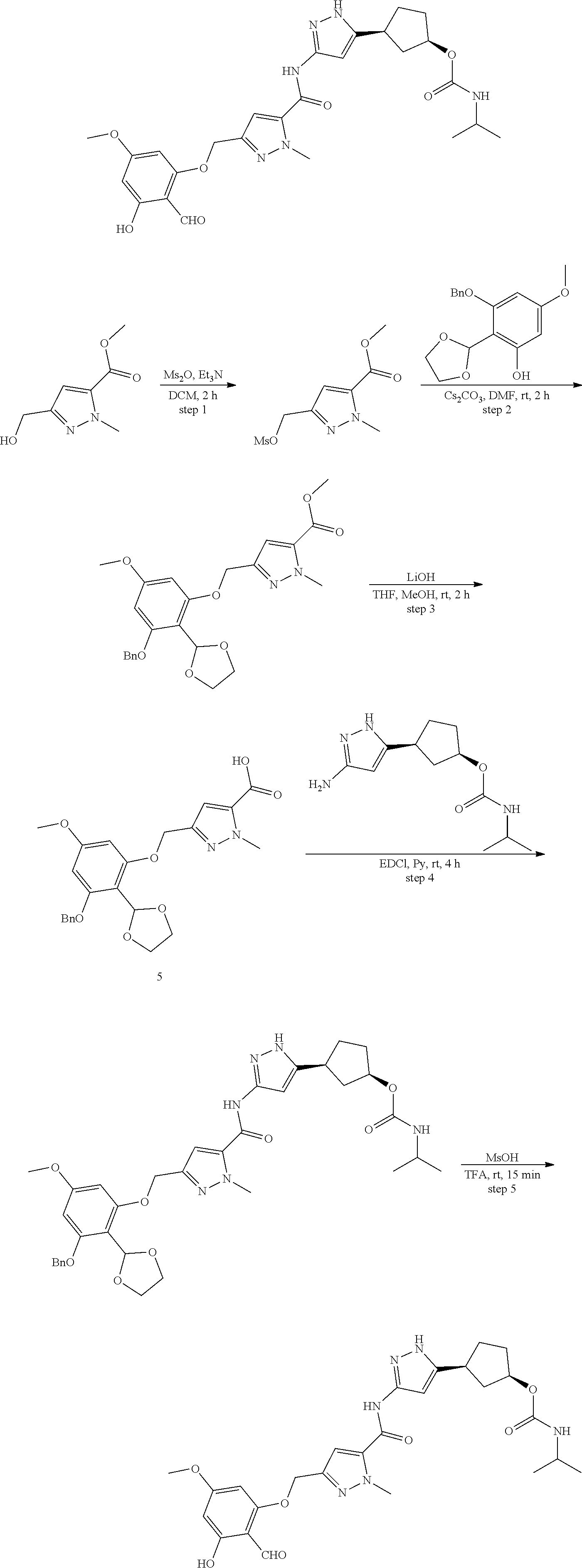

### Step 1: Synthesis of methyl 5-[(methanesulfonyloxy)methyl]-2-methylpyrazole-3-carboxylate

Methyl 5-(hydroxymethyl)-2-methylpyrazole-3-carboxylate (1 g, 5.877 mmol), Et₃N (0.9 g, 8.894 mmol), a stir bar and DCM (20 mL) were added to a 100 mL round-bottom flask and stirred until homogeneous, and then treated with Ms₂O (1.23 g, 7.052 mmol). The reaction mixture was stirred for 2 h at r.t, then diluted with water. The resulting mixture was extracted with DCM (2×100 mL). The combined organic layers were washed with brine, dried over anhydrous sodium sulfate, filtered, and concentrated under vacuum. The residue was purified by silica gel chromatography (0-45% EA/PE) to afford methyl 5-[(methanesulfonyloxy)methyl]-2-methylpyrazole-3-carboxylate as colorless oil (1 g, 82.26% yield). MS (ESI) mass calcd. for C₈H₁₂N₂O₅S, 248.05 m/z, found, 248.95 [M+H]⁺.

### Step 2: Synthesis of methyl 5-[3-(benzyloxy)-2-formyl-5-methoxyphenoxymethyl]-2-methylpyrazole-3-carboxylate

Methyl 5-[(methanesulfonyloxy)methyl]-2-methylpyrazole-3-carboxylate (313 mg, 1.261 mmol), 2-(benzyloxy)-6-hydroxy-4-methoxybenzaldehyde (250 mg, 0.968 mmol), a stir bar and DMF (5 mL) were added to a 40 vial and stirred until homogeneous, then treated with Cs₂CO₃ (790 mg, 2.417 mmol). The reaction mixture was stirred for 2 h at r.t, then diluted with water. The resulting mixture was extracted with EA (2×100 mL). The combined organic layers were washed with brine, dried over anhydrous sodium sulfate, filtered, and concentrated under vacuum. The residue was purified by silica gel chromatography (0-65% EA/PE) to afford methyl 5-[3-(benzyloxy)-2-formyl-5-methoxyphenoxymethyl]-2-methylpyrazole-3-carboxylate (390 mg, 98.17% yield). MS (ESI) mass calcd. for C₂₂H₂₂N₂O₆, 410.15 m/z, found, 411.15 [M+H]⁺.

### Step 3: Synthesis of 5-[3-(benzyloxy)-2-formyl-5-methoxyphenoxymethyl]-2-methylpyrazole-3-carboxylic acid

Methyl 5-[3-(benzyloxy)-2-formyl-5-methoxyphenoxymethyl]-2-methylpyrazole-3-carboxylate (390 mg, 0.950 mmol), a stir bar, THF (3 mL) and MeOH (1 mL) were added to a 50 mL round-bottom flask and stirred until homogeneous, and then treated with LiOH (1 mL, 2.851 mmol, 3 mol/L). The reaction mixture was stirred for 2 h at r.t, then diluted with water (1 mL) and concentrated under vacuum. The pH of the resulting mixture was adjusted to 4-5 with 2 M HCl. The resulting mixture was extracted with EA (2×80 mL). The combined extracts were washed with brine, dried over anhydrous sodium sulfate, filtered, and concentrated under vacuum to afford 5-[3-(benzyloxy)-2-formyl-5-methoxyphenoxymethyl]-2-methylpyrazole-3-carboxylic acid as a white solid (380 mg, crude). MS (ESI) mass calcd. for C₂₁H₂₀N₂O₆, 396.13 m/z, found, 395.05 [M−H]⁻.

### Step 4: Synthesis of (1R,3S)-3-(5-(5-[3-(benzyloxy)-2-formyl-5-methoxyphenoxymethyl]-2-methylpyrazole-3-amido-2H-pyrazol-3-yl)cyclopentyl N-isopropylcarbamate

(1R,3S)-3-(5-amino-2H-pyrazol-3-yl)cyclopentyl N-isopropylcarbamate (120 mg, 0.476 mmol), a stir bar, pyridine (5 mL) were added to a 20 mL vial and stirred until homogeneous, then treated with EDCI (137 mg, 0.715 mmol), 5-[3-(benzyloxy)-2-formyl-5-methoxyphenoxymethyl]-2-methylpyrazole-3-carboxylic acid (250 mg, 0.631 mmol). The reaction mixture was stirred for 4 h at r.t and concentrated under vacuum. The residue was purified by reverse-phase chromatography (5-60% ACN/H₂O 10 mM NH₄HCO₃) to afford (1R,3S)-3-(5-(5-[3-(benzyloxy)-2-formyl-5-methoxyphenoxymethyl]-2-methylpyrazole-3-amido-2H-pyrazol-3-yl)cyclopentyl N-isopropyl carbamate as a yellow solid (60 mg, 20.00% yield). MS (ESI) mass calcd. for C₃₃H₃₈N₆O₇, 630.28 m/z, found, 631.25 [M+H]⁺.

### Step 4: Synthesis of (1R,3S)-3-(5-[5-(2-formyl-3-hydroxy-5-methoxyphenoxymethyl)-2-methylpyrazole-3-amido]-2H-pyrazol-3-ylcyclopentyl N-isopropylcarbamate

(1R,3S)-3-(5-(5-[3-(benzyloxy)-2-formyl-5-methoxyphenoxymethyl]-2-methylpyrazole-3-amido-2H-pyrazol-3-yl)cyclopentyl N-isopropylcarbamate (60 mg, 0.095 mmol), a stir bar and TFA (2.5 mL) were added to a 8 mL vial and stirred until homogeneous, and then treated with MsOH (0.8 mL). The resulting mixture was stirred for 15 min at room temperature, then concentrated under vacuum and purified by Prep-HPLC with (Column: Xselect CSH Prep C18 OBD Column, 30×150 mm, 5 um; Mobile Phase A: Water (0.05% TFA), Mobile Phase B: ACN; Flow rate: 60 mL/min; Gradient: 36% B to 58% B in 10 min; Wave Length: 25/220 nm to afford (1R,3S)-3-(5-[5-(2-formyl-3-hydroxy-5-methoxyphenoxymethyl)-2-methylpyrazole-3-amido]-2H-pyrazol-3-ylcyclopentyl N-isopropylcarbamate as a yellow solid (10.3 mg, 19.18% yield, MS (ESI) mass calcd. for C₂₆H₃₂N₆O₇, 540.23 m/z, found, 541.25 [M+H]⁺). ¹H NMR (400 MHz, DMSO-d₆) δ 12.36 (s, 1H), 10.81 (s, 1H), 10.09 (s, 1H), 7.30 (s, 1H), 6.97 (d, J=8.0 Hz, 1H), 6.42 (s, 1H), 6.39-6.31 (m, 1H), 6.19-6.10 (m, 1H), 5.18 (s, 2H), 5.00 (s, 1H), 4.09 (s, 4H), 3.85 (s, 3H), 3.64-3.51 (m, 1H), 3.16-2.99 (m, 1H), 2.09-1.96 (m, 1H), 1.96-1.81 (m, 1H), 1.80-1.53 (m, 3H), 1.03 (d, J=6.5 Hz, 6H).

### Example 4.32: Synthesis of Compound 70: (1R,3S)-3-{5-[6-(2-formyl-3-hydroxyphenoxy)-3-methylpyrazine-2-amido]-2H-pyrazol-3-yl}cyclopentyl N-isopropylcarbamate

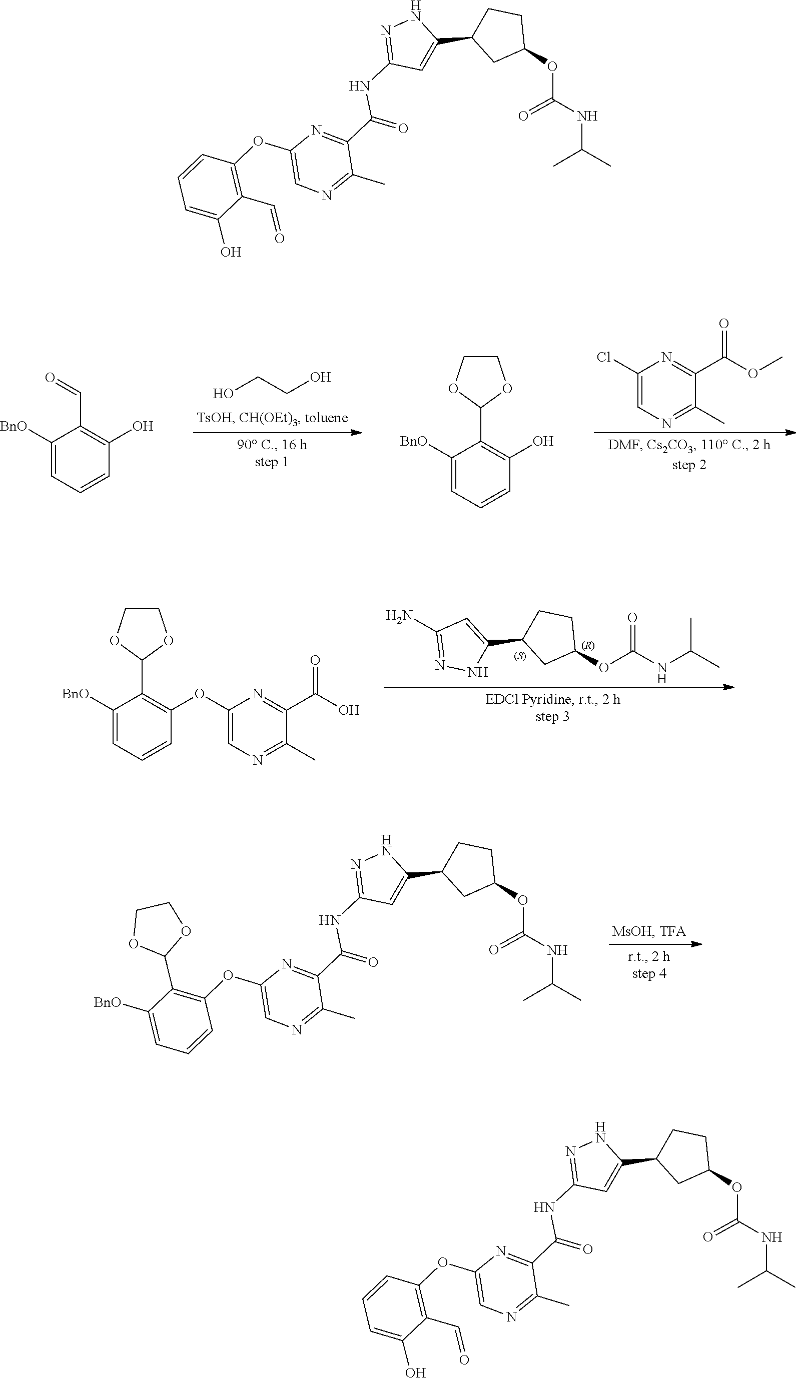

### Step 1: Synthesis of 3-(benzyloxy)-2-(1,3-dioxolan-2-yl)phenol

2-(benzyloxy)-6-hydroxybenzaldehyde (2 g, 8.762 mmol), triethyl orthoformate (3.9 g, 26.286 mmol), and toluene (20 mL, 187.973 mmol) were added to a 250 mL round-bottom flask and stirred until homogeneous, then treated with TsOH (0.15 g, 0.876 mmol). The reaction mixture was stirred at 90° C. overnight then concentrated under vacuum. The residue was purified by silica gel chromatography (0-40% PE/EA) to afford 3-(benzyloxy)-2-(1,3-dioxolan-2-yl)phenol as a yellow oil (1.4 g, 58.68%). MS (ESI) mass calcd. for C₁₆H₁₆O₄, 272.10 m/z, found 273.15 [M+H]⁺.

### Step 2: Synthesis of 6-[3-(benzyloxy)-2-(1,3-dioxolan-2-yl)phenoxy]-3-methylpyrazine-2-carboxylic acid

A solution of 3-(benzyloxy)-2-(1,3-dioxolan-2-yl)phenol (430 mg, 1.579 mmol), methyl 6-chloro-3-methylpyrazine-2-carboxylate (589 mg, 3.158 mmol), and Cs₂CO₃ (3.1 g, 9.474 mmol) in DMF (6 mL) was heated to 110° C. for 2 h under an atmosphere of N₂. The mixture was acidified to pH=3 with HCl (2 M). The resulting mixture was diluted with H₂O and extracted with EA. The residue was purified by reverse-phase chromatography (5-60%, ACN/H₂O with 5 mM TFA) to afford 6-[3-(benzyloxy)-2-(1,3-dioxolan-2-yl)phenoxy]-3-methylpyrazine-2-carboxylic acid (218 mg, 33.80%) as a yellow solid. MS (ESI) mass calcd. for C₂₂H₂₀N₂O₆, 408.13 m/z, found 409.10 [M+H]⁺.

### Step 3: Synthesis of (1R,3S)-3-(5-{6-[3-(benzyloxy)-2-(1,3-dioxolan-2-yl)phenoxy]-3-methylpyrazine-2-amido}-2H-pyrazol-3-yl)cyclopentyl N-isopropylcarbamate

6-[3-(benzyloxy)-2-(1,3-dioxolan-2-yl)phenoxy]-3-methylpyrazine-2-carboxylic acid (168 mg, 0.412 mmol), (1R,3S)-3-(5-amino-2H-pyrazol-3-yl)cyclopentyl N-isopropylcarbamate (80 mg, 0.317 mmol) and pyridine (3 mL) were added to a 40 mL vail and stirred until homogeneous, then treated with EDCI (72 mg, 0.380 mmol). The reaction mixture was stirred at rt. For 2 h. The reaction was quenched with H₂O and extracted with EA. The combined organic layers were washed with brine, dried over anhydrous sodium sulfate, filtered, and concentrated under vacuum. The residue was purified by reverse-phase chromatography (5-60%, ACN/H₂O with 5 mM TFA water) to afford (1R,3S)-3-(5-{6-[3-(benzyloxy)-2-(1,3-dioxolan-2-yl)phenoxy]-3-methylpyrazine-2-amido}-2H-pyrazol-3-yl)cyclopentyl N-isopropylcarbamate (150 mg, 73.61%) as a white solid. MS (ESI) mass calcd. for C₃₄H₃₈N₆O₇, 642.28 m/z, found 643.10 [M+H]⁺.

### Step 4: Synthesis of (1R,3S)-3-{5-[6-(2-formyl-3-hydroxyphenoxy)-3-methylpyrazine-2-amido]-2H-pyrazol-3-yl}cyclopentyl N-isopropylcarbamate

(1R,3S)-3-(5-{6-[3-(benzyloxy)-2-(1,3-dioxolan-2-yl)phenoxy]-3-methylpyrazine-2-amido}-2H-pyrazol-3-yl)cyclopentyl N-isopropylcarbamate (90 mg, 0.140 mmol), methanesulfonic acid (1.2 mL) and trifluoroacetic acid (3.6 mL) were added to a 20 mL vail and stirred until homogeneous. The reaction mixture was stirred at rt. For 2 h and concentrated under vacuum. The residue was purified by Prep-HPLC (Column: Xselect CSH Prep C18 OBD Column, 30×150 mm, 5 um; Mobile Phase A: Water (0.05% TFA), Mobile Phase B: ACN; Flow rate: 60 mL/min; Gradient: 34% B to 56% B in 10 min; Wave Length: 254/220 nm) to afford (1R,3S)-3-{5-[6-(2-formyl-3-hydroxyphenoxy)-3-methylpyrazine-2-amido]-2H-pyrazol-3-yl}cyclopentyl N-isopropyl carbam-ate as a yellow solid (8.0 mg, 11.23%). MS (ESI) mass calcd. for C₂₅H₂₈N₆O₆, 508.20 m/z, found 509.20 [M+H]⁺. ¹H NMR (400 MHz, DMSO-d₆) δ 11.35 (s, 1H), 10.32-10.21 (m, 2H), 8.69-8.62 (m, 1H), 7.60 (t, J=8.3 Hz, 1H), 7.00-6.94 (m, 1H), 6.94-6.88 (m, 1H), 6.85-6.75 (m, 1H), 6.46-6.36 (m, 1H), 5.04-4.95 (m, 1H), 3.62-3.49 (m, 1H), 3.12-3.02 (m, 1H), 2.72-2.62 (m, 3H), 2.49-2.43 (m, 1H), 2.09-1.53 (m, 6H), 1.03 (d, J=6.6 Hz, 6H).

### Example 4.33: Synthesis of Compound 110: (1s,3s)-3-(3-(2-(2-formyl-3,5-dimethoxyphenoxy)acetamido)-1H-pyrazol-5-yl)cyclobutyl propylcarbamate

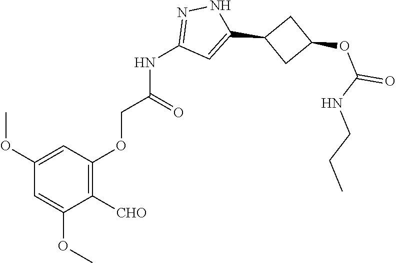 [SMILES: CCCNC(=O)O[C@H]1C[C@@H](c2cc(NC(=O)COc3cc(OC)cc(OC)c3C=O)n[nH]2)C1]

### Synthetic Route

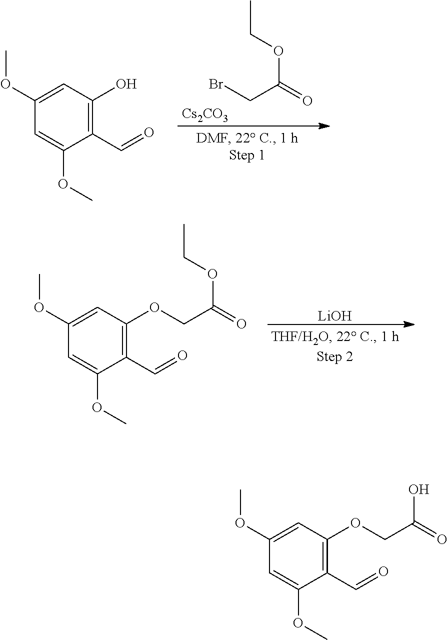

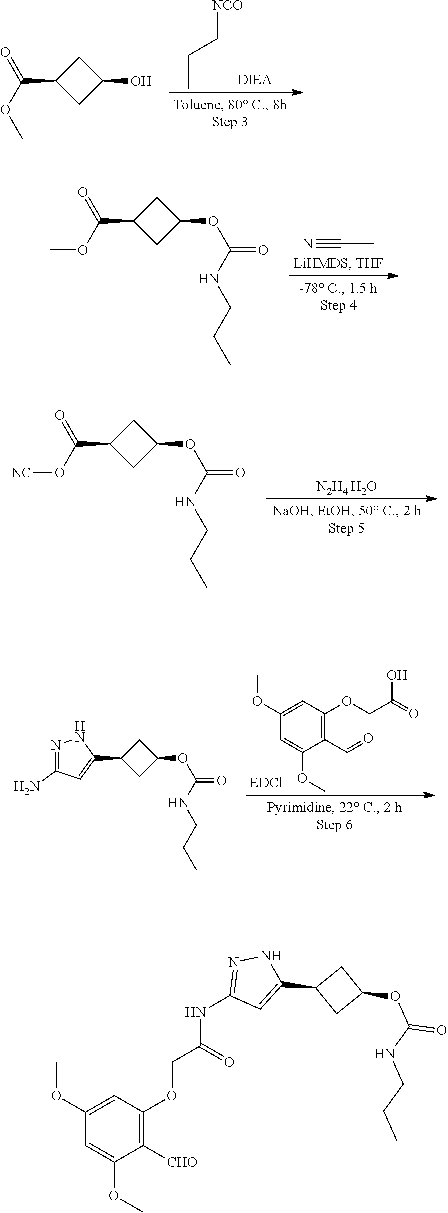

### Step 1: Synthesis of ethyl 2-(2-formyl-3,5-dimethoxyphenoxy)acetate

A solution of 2-hydroxy-4,6-dimethoxybenzaldehyde (10 g, 54.892 mmol, 1 equiv) and Cs₂CO₃ (35.7 g, 109.784 mmol, 2 equiv) and ethyl bromoacetate (9.17 g, 54.892 mmol, 1 equiv) in DMF (30 mL) were stirred for 1 h at 22° C. The resulting mixture was diluted with EA (300 mL) and washed with H₂O (3×60 mL). The combined organic layers were washed with brine (60 mL) and dried over anhydrous Na₂SO₄. After filtration, the filtrate was concentrated under reduced pressure. The residue was purified by silica gel column chromatography, eluted with EA/PE (50-70%) to afford ethyl 2-(2-formyl-3,5-dimethoxyphenoxy)acetate (8 g, 51.61%) as a black oil. MS (ESI) calcd. for C₁₃H₁₆O₆, 268.08 m/z, found 269.00 [M+H]⁺.

### Step 2: Synthesis of 2-formyl-3,5-dimethoxyphenoxyacetic acid

A solution of ethyl 2-(2-formyl-3,5-dimethoxyphenoxy)acetate (672 mg, 2.505 mmol, 1 equiv) and LiOH (90 mg, 3.757 mmol, 1.5 equiv) in THF/H₂O (5 mL: 1 mL) was stirred for 1 h at 22° C. The mixture acidified to pH ˜5 with HCl (0.5 mol/L). The aqueous layer was extracted with DCM (3×10 mL). The combined organic layers were washed with brine (50 mL), dried over anhydrous Na₂SO₄. After filtration, the filtrate was concentrated under reduced pressure. This resulted in crude 2-formyl-3,5-dimethoxyphenoxyacetic acid (600 mg, 94.73%) as a yellow solid which was used without purification. MS (ESI) calcd. for C₁₁H₁₂O₆, 240.06 m/z, found 241.16 [M+H]⁺.

### Step 3: Synthesis of methyl (1s,3s)-3-[(propylcarbamoyl)oxy]cyclobutane-1-carboxylate

A solution of methyl (1s,3s)-3-hydroxycyclobutane-1-carboxylate (1.7 g, 13.063 mmol, 1 equiv), 1-isocyanatopropane (4.45 g, 52.252 mmol, 4 equiv) and DIEA (6.75 g, 52.252 mmol, 4 equiv) in toluene (20 mL) was stirred for 8 h at 80° C. The resulting mixture was concentrated under reduced pressure. The residue was purified by silica gel column chromatography, eluted with MeOH/DCM (0-20%) to afford methyl (1s,3s)-3-((propylcarbamoyl)oxy)cyclobutane-1-carboxylate (3 g, 96.03%) as a brown solid. MS (ESI) calcd. for C₁₀H₁₇NO₄, 215.11 m/z, found 216.11 [M+H]⁺.

### Step 4: Synthesis of (1s,3s)-3-(2-cyanoacetyl)cyclobutyl propylcarbamate

A solution of methyl (1s,3s)-3-((propylcarbamoyl)oxy)cyclobutane-1-carboxylate (50 mg, 0.206 mmol, 1.0 equiv) in THF (20 mL) was added acetonitrile (0.86 g, 20.907 mmol, 3 equiv) and cooled to −78° C., then LiHMDS (13.94 mL, 13.938 mmol, 2 equiv) was added dropwise over 5 minutes at −78° C. under nitrogen atmosphere. The resulting mixture was stirred for 1.5 hour at −78° C. under N₂ atmosphere. The reaction was quenched by the addition of saturated solution of NH₄Cl (200 mL) at −78° C. The resulting mixture was let warm to room temperature and extracted with EA (3×100 mL). The combined organic layers were washed with brine (100 mL), dried over anhydrous Na₂SO₄. After filtration, the filtrate was concentrated under reduced pressure. The residue was purified by silica gel column chromatography, eluted with MeOH/DCM (0-20%) to afford (1s,3s)-3-(2-cyanoacetyl)cyclobutyl propylcarbamate (1.6 g, 92.14%) as a brown solid. MS (ESI) calcd. for C₁₁H₁₆N₂O₃, 224.11 m/z, found 225.11 [M+H]⁺.

### Step 5: Synthesis of (1s,3s)-3-(3-amino-1H-pyrazol-5-yl)cyclobutyl propylcarbamate

A solution of (1s,3s)-3-(2-cyanoacetyl)cyclobutyl propylcarbamate (800 mg, 3.567 mmol, 1 equiv) and hydrazinium hydroxide solution (1.75 g, 35.670 mmol, 10 equiv) in EtOH (15 mL) was stirred for 2 h at 50° C. After completion, the resulting mixture was concentrated under reduced pressure. The residue was purified by silica gel column chromatography, eluted with MeOH/DCM (0-20%) to afford (1s,3s)-3-(3-amino-1H-pyrazol-5-yl)cyclobutyl propylcarbamate (400 mg, 44.70%) as a white solid. MS (ESI) calcd. for C₁₁H₁₈N₄O₂, 238.14 m/z, found 239.14 [M+H]⁺.

### Step 6: (1s,3s)-3-(3-(2-(2-formyl-3,5-dimethoxyphenoxy)acetamido)-1H-pyrazol-5-yl)cyclobutyl propylcarbamate

A solution of (1s,3s)-3-(3-amino-1H-pyrazol-5-yl)cyclobutyl propylcarbamate (100 mg, 0.420 mmol, 1 equiv), 2-(2-formyl-3,5-dimethoxyphenoxy)acetic acid (151.21 mg, 0.630 mmol, 1.5 equiv) and EDCI (104.58 mg, 0.546 mmol, 1.3 equiv) in pyridine (2 mL) was stirred for 2 h at 22° C. The mixture was purified by Prep-HPLC under the condition: Column: XSelect CSH Fluoro Phenyl 30×150 mm, 5 μm; Mobile Phase A: Water (0.1% FA), Mobile Phase B: ACN; Flow rate: 60 mL/min; Gradient: 28% B to 45% B in 10 min; Wave Length: 254/220 nm to afford (1s,3s)-3-(3-(2-(2-formyl-3,5-dimethoxyphenoxy)acetamido)-1H-pyrazol-5-yl)cyclobutyl propylcarbamate (8 mg, 3.95%) as a yellow solid. MS (ESI) calcd. for C₂₂H₂₈N₄O₇, 460.19 m/z, found 461.19 [M+H]⁺. ¹H NMR (400 MHz, DMSO-d₆) δ 12.26 (s, 1H), 10.36 (s, 1H), 10.27 (s, 1H), 7.39-6.97 (m, 1H), 6.41-6.30 (m, 3H), 4.95-4.67 (m, 3H), 3.88 (d, J=3.2 Hz, 6H), 3.13-3.02 (m, 1H), 2.92 (q, J=6.7 Hz, 2H), 2.74-2.63 (m, 2H), 2.19-1.98 (m, 2H), 1.40 (h, J=7.3 Hz, 2H), 0.83 (t, J=7.4 Hz, 3H).

### Example 4.34: Synthesis of Compound 112: (1s,3s)-3-(3-(2-(2-formyl-3-hydroxy-5-methoxyphenoxy)acetamido)-1H-pyrazol-5-yl)cyclobutyl pyrrolidine-1-carboxylate

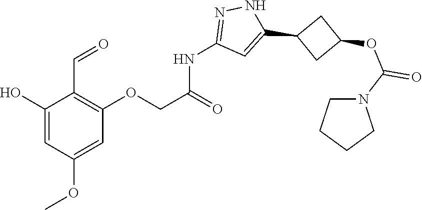 [SMILES: COc1cc(O)c(C=O)c(OCC(=O)Nc2cc([C@H]3C[C@@H](OC(=O)N4CCCC4)C3)[nH]n2)c1]

### Synthetic Route

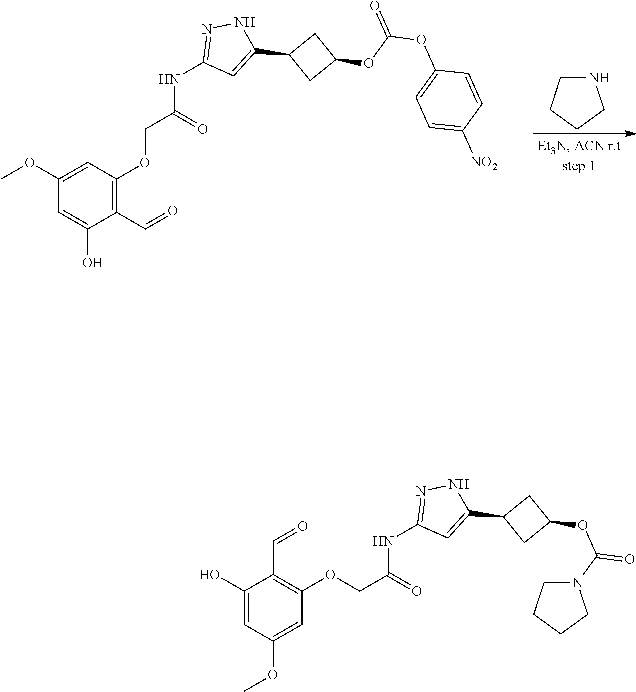 [SMILES: COc1cc(O)c(C=O)c(OCC(=O)Nc2cc([C@H]3C[C@@H](OC(=O)Oc4ccc([N+](=O)[O-])cc4)C3)[nH]n2)c1 ; C1CCNC1 ; COc1cc(O)c(C=O)c(OCC(=O)Nc2cc([C@H]3C[C@@H](OC(=O)N4CCCC4)C3)[nH]n2)c1]

The Synthesis of the starting material was described for compound 111.

### Step 1: Synthesis of (1s,3s)-3-(3-(2-(2-formyl-3-hydroxy-5-methoxyphenoxy)acetamido)-1H-pyrazol-5-yl)cyclobutyl pyrrolidine-1-carboxylate

(1s,3s)-3-(3-(2-(2-formyl-3-hydroxy-5-methoxyphenoxy)acetamido)-1H-pyrazol-5-yl)cyclobutyl (4-nitrophenyl) carbonate (130 mg, 0.247 mmol, 1 equiv), a stir bar, Et₃N (58 mg, 0.573 mmol, 2.32 equiv) and ACN (3.5 mL) were added to a 20 mL vial and stirred until homogenous, then treated with a solution of pyrrolidine (11 mg, 0.155 mmol, 0.63 equiv) in ACN (0.5 mL) dropwise at 0° C. The resulting mixture was stirred for 2 h at room temperature, diluted with water (5 mL) and extracted with DCM (30 mL×3). The combined organic layers were washed with brine, dried over anhydrous sodium sulfate, filtered, and concentrated under vacuum pressure. The residue was purified by silica gel chromatography (0-7% DCM/MeOH) to afford a crude product as a green oil, which was further purified by Column: XSelect CSH Fluoro Phenyl 30*150 mm, 5 μm; Mobile Phase A: Water (0.05% TFA), Mobile Phase B: ACN; Flow rate: 60 mL/min mL/min; Gradient: 29-46% B in 10 min; Wave Length: 254/220 nm nm; RT1 (min): 8.92 to give (1s,3s)-3-(3-(2-(2-formyl-3-hydroxy-5-methoxyphenoxy)acetamido)-1H-pyrazol-5-yl)cyclobutyl pyrrolidine-1-carboxylate (9.7 mg, 8.46%) as a white solid. MS (ESI) mass cacld. for C₂₂H₂₆N₄O₇, 458.18 m/z, found, 459.25 [M+H]⁺. 1H NMR (400 MHz, DMSO-d₆) δ 12.32 (s, 1H), 10.56 (s, 1H), 10.15 (s, 1H), 6.35 (s, 1H), 6.14 (d, J=2.1 Hz, 1H), 6.07 (d, J=2.2 Hz, 1H), 4.88-4.76 (m, 3H), 3.81 (s, 3H), 3.32-3.25 (m, 2H), 3.25-3.18 (m, 2H), 3.14-3.00 (m, 1H), 2.73-2.61 (m, 2H), 2.15-2.03 (m, 2H), 1.84-1.73 (m, 4H).

### Example 4.35: Synthesis of Compound 128: (1R,3S)-3-(3-(2-(2-formyl-3-hydroxy-5-methoxyphenoxy)acetamido)-1H-pyrazol-5-yl)cyclopentyl tert-butylcarbamate

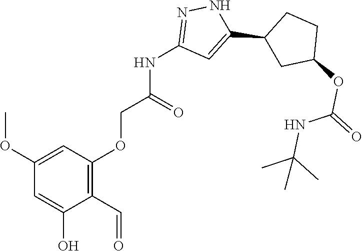 [SMILES: COc1cc(O)c(C=O)c(OCC(=O)Nc2cc([C@H]3CC[C@@H](OC(=O)NC(C)(C)C)C3)[nH]n2)c1]

### Synthetic Route

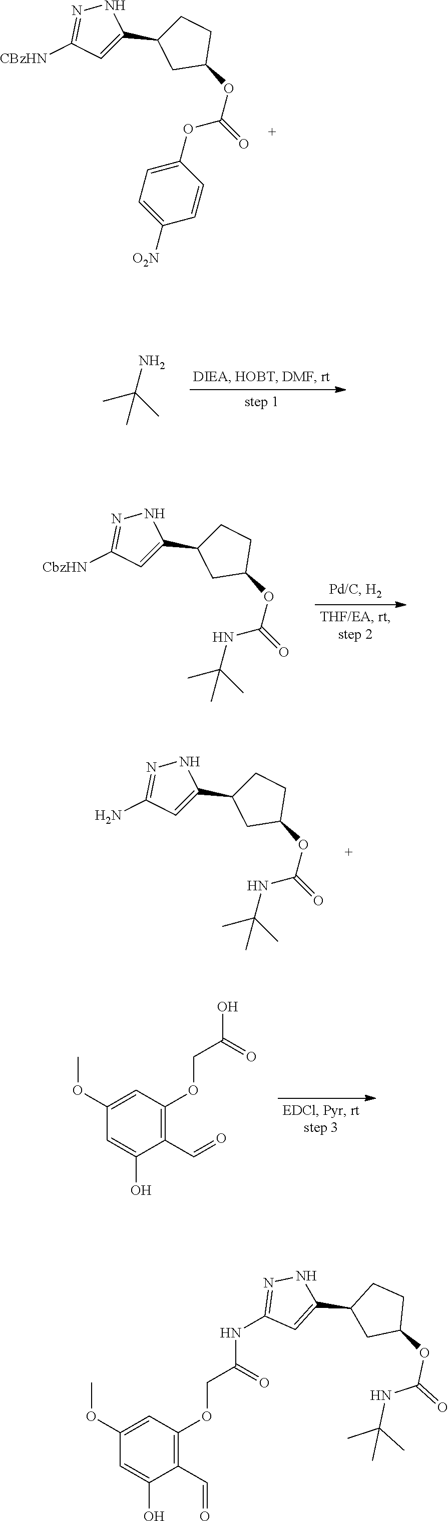

### Step 1: Synthesis of benzyl (5-((1S,3R)-3-((tert-butylcarbamoyl)oxy)cyclopentyl)-1H-pyrazol-3-yl)carbamate

Intermediate C (1 g, 2.144 mmol, 1 equiv), 2-methylpropan-2-amine (0.35 g, 4.781 mmol, 2 equiv), a stir bar, DMF (10 mL) were added to a 40 vial, stirred until homogeneous, then treated with DIEA (1.2 g, 9.285 mmol, 4 equiv) and, HOBT (0.6 g, 4.438 mmol, 2 equiv). The resulting mixture was maintained under nitrogen and stirred for 3 h at r.t, then diluted with water and extracted with EA (100 mL) twice. The combined organic extracts were washed with brine, dried over anhydrous sodium sulfate, filtered, and concentrated in vacuo. The residue was purified by reverse-phase column chromatography, 5-50% ACN/water with 10 mM NH₄HCO₃ to afford benzyl (5-((1S,3R)-3-((tert-butylcarbamoyl)oxy)cyclopentyl)-1H-pyrazol-3-yl)carbamate as a yellow solid (450 mg, 52.4% yield).

### Step 2: Synthesis of (1R,3S)-3-(3-amino-1H-pyrazol-5-yl)cyclopentyl tert-butylcarbamate

Benzyl (5-((1S,3R)-3-((tert-butylcarbamoyl)oxy)cyclopentyl)-1H-pyrazol-3-yl)carbamate (450 mg, 1.124 mmol, 1 equiv), 10% Pd/C (300 mg) a stir bar, EA (8 mL) and THF (8 mL) were added to a 100 round-bottom flask. The resulting mixture was maintained under H₂ and stirred for 3 h at r.t, then filtered and washed with THF (10 mL). The filtrate was concentrated in vacuo to afford (1R,3S)-3-(3-amino-1H-pyrazol-5-yl)cyclopentyl tert-butylcarbamate as a yellow solid (317 mg, crude). The crude material was used without any further purification.

### Step 3: Synthesis of (1R,3S)-3-(3-(2-(2-formyl-3-hydroxy-5-methoxyphenoxy)acetamido)-1H-pyrazol-5-yl)cyclopentyl tert-butylcarbamate

(1R,3S)-3-(3-amino-1H-pyrazol-5-yl)cyclopentyl tert-butylcarbamate (200 mg, 0.751 mmol, 1 equiv), intermediate A (170 mg, 0.752 mmol, 1 equiv) a stir bar, pyridine (8 mL) was added to a 20 vial and stirred until homogeneous, and then treated with EDCI (190 mg, 0.991 mmol, 1.3 equiv). The resulting mixture was maintained under nitrogen and stirred for 2 h at r.t, then concentrated in vacuo to a crude product, which was further purified by HPLC, 29 to 49% MeCN/water with 0.05% TFA modifier to afford (1R,3S)-3-(3-(2-(2-formyl-3-hydroxy-5-methoxyphenoxy)acetamido)-1H-pyrazol-5-yl)cyclopentyl tert-butylcarbamate as an off-white solid (25.4 mg, 6.9% yield). ¹H NMR (400 MHz, DMSO-D6) δ 12.32 (s, 1H), 10.57 (s, 1H), 10.15 (s, 1H), 6.76 (s, 1H), 6.31 (s, 1H), 6.19-6.03 (m, 2H), 4.96 (s, 1H), 4.81 (s, 2H), 3.81 (s, 3H), 3.15-2.94 (m, 1H), 2.48-2.42 (m, 1H), 2.04-1.78 (m, 2H), 1.77-1.65 (m, 2H), 1.64-1.47 (m, 1H), 1.19 (s, 9H). MS (ESI) mass calcd. for C₂₃H₃₀N₄O₇ 474.21 m/z, found, [M+H]⁺ 475.25 m/z.

### Example 4.36: Synthesis of Compound 130: (1R,3S)-3-(3-((R)-2-(2-formyl-3-hydroxy-5-methoxyphenoxy)propanamido)-1H-pyrazol-5-yl)cyclopentyl (1-methylcyclopropyl)carbamate

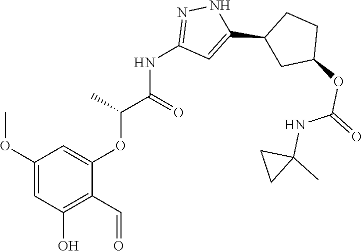 [SMILES: COc1cc(O)c(C=O)c(O[C@H](C)C(=O)Nc2cc([C@H]3CC[C@@H](OC(=O)NC4(C)CC4)C3)[nH]n2)c1]

### Synthetic Route

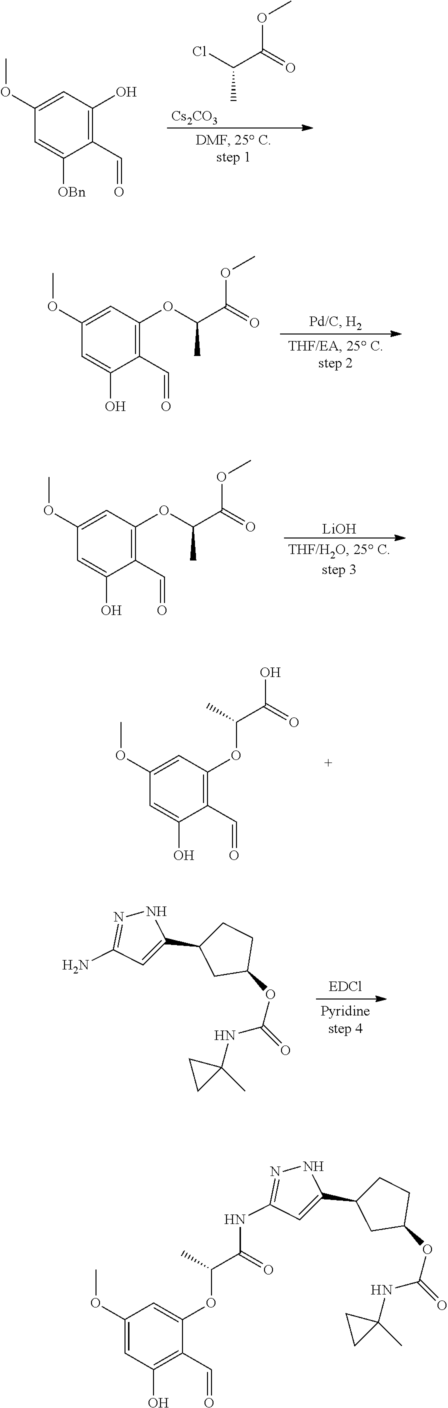

### Step 1: Methyl (R)-2-(3-(benzyloxy)-2-formyl-5-methoxyphenoxy)propanoate

To a stirred solution of 2-(benzyloxy)-6-hydroxy-4-methoxybenzaldehyde (3.0 g, 11.61 mmol, 1.0 equiv) in DMF (30 mL) was added Cs₂CO₃ (9.4 g, 29.04 mmol, 2.5 equiv) at 25° C. and stirred for 0.5 h. Then to the above mixture was added methyl (2S)-2-chloropropanoate (2.1 g, 17.42 mmol, 1.5 equiv) at 25° C. The resulting mixture was stirred for an additional 9 h at 25° C. The mixture was diluted with EA (30 mL), washed with water (20 mL×3) and brine (30 mL), dried over anhydrous sodium sulfate, and concentrated in vacuo. The residue was purified by silica gel column chromatography, 0 to 100% EA/PE to afford methyl (R)-2-(3-(benzyloxy)-2-formyl-5-methoxyphenoxy)propanoate (2.1 g, 6.08 mmol, 50.81% yield) as a yellow oil. MS (ESI) calcd. for C₁₉H₂₀O₆, 344.13 m/z, found [M+H]⁺ 345.05 m/z.

### Step 2: Methyl (R)-2-(2-formyl-3-hydroxy-5-methoxyphenoxy)propanoate

A solution of methyl (R)-2-(3-(benzyloxy)-2-formyl-5-methoxyphenoxy)propanoate (1.0 g, 2.90 mmol, 1.0 equiv) and Pd/C (0.4 g, 3.77 mmol, 1.3 equiv, 10% on active carbon) in THF (10 mL) and EA (10 mL) was stirred at 25° C. under hydrogen atmosphere for 3 h. The resulting mixture was filtered and the filtrate was concentrated under reduced pressure. This resulted in methyl (R)-2-(2-formyl-3-hydroxy-5-methoxyphenoxy)propanoate (900.0 mg, 3.529 mmol, 51.00% yield) as a yellow solid, The crude material was used without any further purification. MS (ESI) calcd. for C₁₂H₁₄O₆, 254.08 m/z, found [M+H]⁺ 255.20 m/z.

### Step 3: (R)-2-(2-formyl-3-hydroxy-5-methoxyphenoxy)propanoic acid

To a stirred solution of methyl (R)-2-(2-formyl-3-hydroxy-5-methoxyphenoxy)propanoate (830.0 mg, 3.26 mmol, 1.0 equiv) and LiOH (93.8 mg, 3.91 mmol, 1.2 equiv) in THF (3 mL) was added water (1 mL) at 25° C. The resulting mixture was stirred at 25° C. for additional 2 h. The mixture was acidified to pH 7 with 1 N HCl at 0° C. The mixture was diluted with EA (30 mL), the organic phase was separated and washed with water (20 mL×3) and brine (10 mL), dried over anhydrous sodium sulfate, and concentrated in vacuo. This resulted in (R)-2-(2-formyl-3-hydroxy-5-methoxyphenoxy)propanoic acid (750.0 mg, 3.11 mmol, 86.07% yield) as a light yellow solid, The crude material was used in the next step without any further purification. MS (ESI) calcd. for C₁₁H₁₂O₆, 240.06 m/z, found [M+H]⁺ 241.00 m/z.

### Step 4: (1R,3S)-3-(3-((R)-2-(2-formyl-3-hydroxy-5-methoxyphenoxy)propanamido)-1H-pyrazol-5-yl)cyclopentyl (1-methylcyclopropyl)carbamate

To a stirred solution of (R)-2-(2-formyl-3-hydroxy-5-methoxyphenoxy)propanoic acid (283.5 mg, 1.18 mmol, 1.3 equiv) and (1R,3S)-3-(3-amino-1H-pyrazol-5-yl)cyclopentyl (1-methylcyclopropyl)carbamate (240.0 mg, 0.90 mmol, 1.0 equiv) in pyridine (1 mL) was added EDCI (348.1 mg, 1.81 mmol, 2.0 equiv) at 25° C. The resulting mixture was stirred for additional 16 h at 25° C. The mixture was purified by reverse phase preparatory phase preparatory HPLC, 10 to 60% ACN/Water with 0.1% FA modifier to afford (1R,3S)-3-(3-((R)-2-(2-formyl-3-hydroxy-5-methoxyphenoxy)propanamido)-1H-pyrazol-5-yl)cyclopentyl (1-methylcyclopropyl)carbamate (88.2 mg, 0.181 mmol, 19.61% yield) as a light yellow solid. ¹H NMR (400 MHz, DMSO-D6) δ 12.34 (s, 1H), 12.17 (s, 1H), 10.67 (s, 1H), 10.14 (s, 1H), 7.34 (s, 1H), 6.28 (s, 1H), 6.12 (d, J=2.1 Hz, 1H), 6.00 (d, J=2.1 Hz, 1H), 5.10-4.95 (m, 2H), 3.77 (s, 3H), 3.06-2.97 (m, 1H), 2.65-2.38 (m, 1H), 1.98 (d, J=9.5 Hz, 1H), 1.86-1.78 (m, 1H), 1.72-1.59 (m, 2H), 1.54 (d, J=6.5 Hz, 4H), 1.22 (s, 3H), 1.16 (s, 2H), 0.58 (s, 2H). MS (ESI) calcd. for C₂₄H₃₀N₄O₇, 486.21 m/z, found [M+H]⁺ 487.20 m/z. (1R,3S)-3-(3-amino-1H-pyrazol-5-yl)cyclopentyl (1-methylcyclopropyl)carbamate was synthesized analogously as to Compound 251.

### Example 4.37: Synthesis of Compound 131: (1R,3S)-3-{5-[2-(2-formyl-3-hydroxy-5-methoxyphenoxy)acetamido]-2H-pyrazol-3-yl}cyclopentyl (3S)-3-cyanopiperidine-1-carboxylate

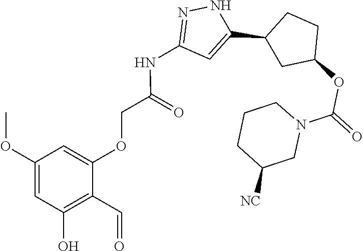 [SMILES: COc1cc(O)c(C=O)c(OCC(=O)Nc2cc([C@H]3CC[C@@H](OC(=O)N4CCC[C@H](C#N)C4)C3)[nH]n2)c1]

### Synthetic Route

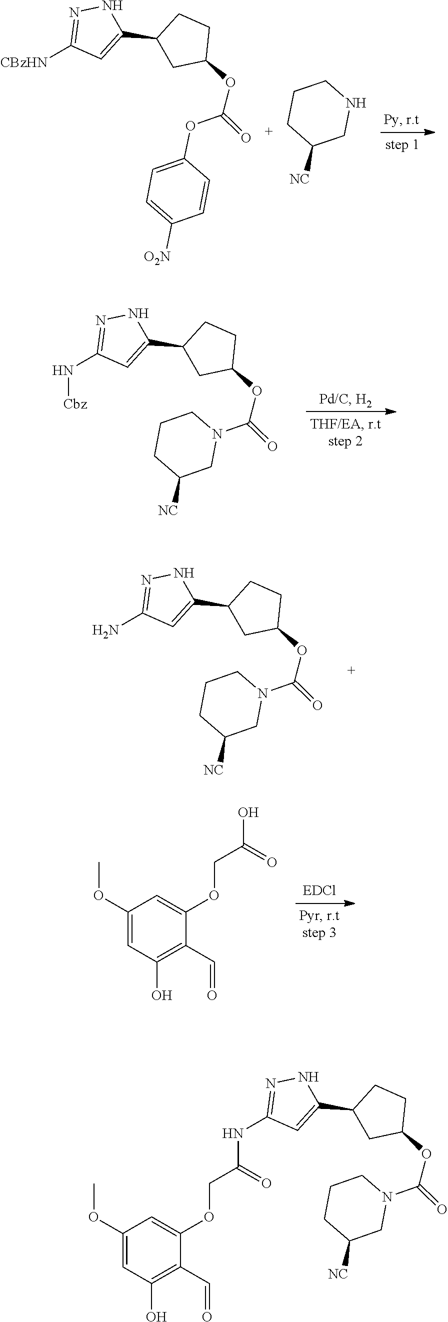

### Step 1: (1R,3S)-3-(3-(((benzyloxy)carbonyl)amino)-1H-pyrazol-5-yl)cyclopentyl (S)-3-cyanopiperidine-1-carboxylate

Intermediate C (700.0 mg, 1.501 mmol, 1 equiv), (S)-piperidine-3-carbonitrile (495.9 mg, 4.503 mmol, 3 equiv) and pyridine (10 mL) was added to a 40 mL glass vial with a stir bar and stirred at r.t for 1 h. The mixture was purified by prep-HPLC, 29 to 42% ACN/water with 0.100 FA modifier to afford (1R,3S)-3-(3-(((benzyloxy)carbonyl)amino)-1H-pyrazol-5-yl)cyclopentyl (S)-3-cyanopiperidine-1-carboxylate (300.0 mg, 0.685 mmol, 41.12% yield) as a colorless solid. MS (ESI) calcd. for C₂₃H₂₇N₅O₄, 437.20 m/z, found [M+H]⁺ 438.35 m/z.

### Step 2:(1R,3S)-3-(3-amino-1H-pyrazol-5-yl)cyclopentyl (S)-3-cyanopiperidine-1-carboxylate

(1R,3S)-3-(3-(((benzyloxy)carbonyl)amino)-1H-pyrazol-5-yl)cyclopentyl (S)-3-cyanopiperidine-1-carboxylate (300.0 mg, 0.686 mmol, 1 equiv), Pd/C (291.8 mg, 2.744 mmol, 4 equiv, 0% on active carbon), THF (5 mL) and EA (5 mL) was added to a 40 mL glass vial with a stir bar and stirred at r.t for 1 h under H₂ atmosphere. The resulting mixture was filtered, the filter cake was washed with THF (10 mL×3). The filtrate was concentrated under reduced pressure. This resulted in (1R,3S)-3-(3-amino-1H-pyrazol-5-yl)cyclopentyl (S)-3-cyanopiperidine-1-carboxylate (200.0 mg, 0.658 mmol, 85.57% yield) as a brown oil, the crude material was used directly without any further purification. MS (ESI) calcd. for C₁₅H₂₁N₅O₂, 303.17 m/z, found [M+H]⁺ 304.20 m/z.

### Step 3: (1R,3S)-3-{5-[2-(2-formyl-3-hydroxy-5-methoxyphenoxy)acetamido]-2H-pyrazol-3-yl}cyclopentyl (3S)-3-cyanopiperidine-1-carboxylate

(1R,3S)-3-(5-amino-2H-pyrazol-3-yl)cyclopentyl (3S)-3-cyanopiperidine-1-carboxylate (200.0 mg, 0.659 mmol, 1 equiv), intermediate A (223.6 mg, 0.989 mmol, 1.5 equiv), EDCI (189.5 mg, 0.989 mmol, 1.5 equiv) and pyridine (5 mL) was added to a 25 mL glass vial with a stir bar and stirred at r.t for 1 h. The reaction mixture was further purified by prep-HPLC, 30 to 42% ACN/water with 0.1% FA modifier to afford (1R,3S)-3-{5-[2-(2-formyl-3-hydroxy-5-methoxyphenoxy)acetamido]-2H-pyrazol-3-yl}cyclopentyl (3S)-3-cyanopiperidine-1-carboxylate (10.2 mg, 0.020 mmol, 2.84% yield) as a light yellow solid. ¹H NMR (400 MHz, DMSO-D6) δ 12.32 (s, 1H), 12.17 (s, 1H), 10.54 (s, 1H), 10.15 (s, 1H), 6.33 (s, 1H), 6.14 (d, J=2.1 Hz, 1H), 6.07 (d, J=2.2 Hz, 1H), 5.08-5.02 (m, 1H), 4.81 (s, 2H), 3.80 (s, 3H), 3.78-3.66 (m, 1H), 3.47 (s, 1H), 3.37-3.26 (m, 1H), 3.12 (d, J=9.4 Hz, 2H), 2.97 (s, 1H), 2.41 (dt, J=14.6, 7.7 Hz, 1H), 2.02 (d, J=9.4 Hz, 1H), 1.90 (s, 1H), 1.78 (s, 5H), 1.50 (s, 2H). MS (ESI) calcd. for C₂₅H₂₉N₅O₇, 511.21 m/z, found [M+H]⁺ 512.35 m/z.

### Example 4.38: Synthesis of Compound 140: (1R,3S)-3-{5-[2-(2-formyl-3-hydroxy-5-methoxyphenoxy)acetamido]-2H-pyrazol-3-yl}cyclopentyl N-[1-(difluoromethyl)cyclopropyl]carbamate

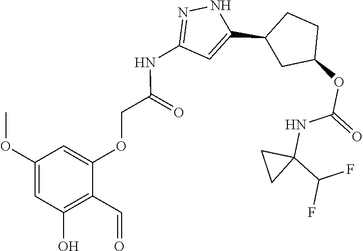 [SMILES: COc1cc(O)c(C=O)c(OCC(=O)Nc2cc([C@H]3CC[C@@H](OC(=O)NC4(C(F)F)CC4)C3)[nH]n2)c1]

### Synthetic Route

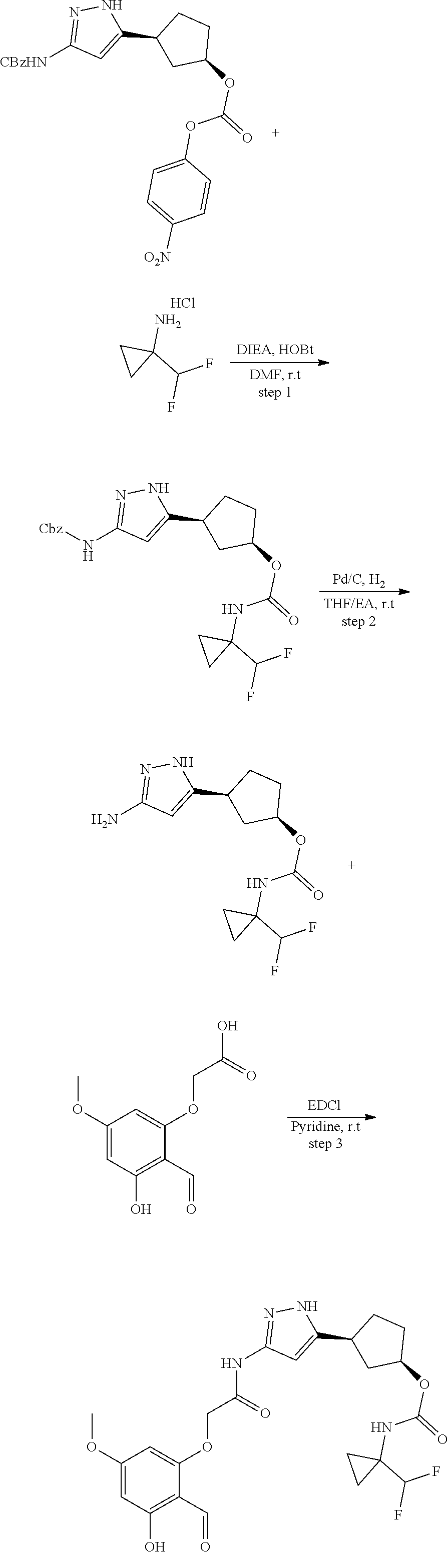

### Step 1: (1R,3S)-3-(3-(((benzyloxy)carbonyl)amino)-1H-pyrazol-5-yl)cyclopentyl (1-(difluoromethyl)cyclopropyl)carbamate

A 25 mL vial was charged with intermediate C (800.0 mg, 1.7 mmol, 1.0 equiv), 1-(difluoromethyl)cyclopropan-1-amine (492.4 mg, 3.4 mmol, 2.0 equiv), DIEA (665.0 mg, 5.1 mmol, 3.0 equiv), 8 mL DMF and HOBt (463.5 mg, 3.4 mmol, 2.0 equiv). The resulting mixture was stirred at r.t for 1 h. The reaction mixture was purified by prep-HPLC, 55-56% MeCN/water with 0.05% FA modifier to afford (1R,3S)-3-(3-(((benzyloxy)carbonyl)amino)-1H-pyrazol-5-yl)cyclopentyl (1-(difluoromethyl)cyclopropyl)carbamate (410.0 mg, 0.9 mmol, 52.3% yield) as a white solid. MS (ESI) calcd. for C₂₁H₂₄F₂N₄O₄, 434.18 m/z, found [M+H]⁺ 435.15 m/z.

### Step 2: (1R,3S)-3-(3-amino-1H-pyrazol-5-yl)cyclopentyl (1-(difluoromethyl)cyclopropyl)carbamate

An oven dried 25 mL vial equipped with a stir bar was charged with (1R,3S)-3-(3-(((benzyloxy)carbonyl)amino)-1H-pyrazol-5-yl)cyclopentyl (1-(difluoromethyl)cyclopropyl)carbamate (400.0 mg, 0.9 mmol, 1.0 equiv), 5 mL EA, 5 mL THF, Pd/C (979.8 mg, 0.9 mmol, 1.0 equiv, 10% on active carbon) and placed under an atmosphere of hydrogen. The resulting mixture was stirred at r.t for 1 h. The resulting mixture was filtered, the filter cake was washed with 10 mL THF. The filtrate was concentrated to dryness to afford (1R,3S)-3-(3-amino-1H-pyrazol-5-yl)cyclopentyl (1-(difluoromethyl)cyclopropyl)carbamate (210.0 mg, 0.5 mmol, 74.4% yield) as a light yellow oil, the crude material was used without any further purification. MS (ESI) calcd. for C₁₃H₁₈F₂N₄O₂, 300.14 m/z, found [M+H]⁺ 301.25 m/z.

### Step 3: (1R,3S)-3-(3-(2-(2-formyl-3-hydroxy-5-methoxyphenoxy)acetamido)-1H-pyrazol-5-yl)cyclopentyl (1-(difluoromethyl)cyclopropyl)carbamate

A 8 mL vial was charged with (1R,3S)-3-(5-amino-2H-pyrazol-3-yl)cyclopentyl N-[1-(difluoromethyl)cyclopropyl]carbamate (140.0 mg, 0.5 mmol, 1.0 equiv), intermediate A (158.2 mg, 0.7 mmol, 1.5 equiv), EDCI (223.4 mg, 1.2 mmol, 2.5 equiv) and 3 mL pyridine. The resulting mixture was stirred at r.t for 1 h. The reaction mixture was diluted with 50 mL water and extracted 3 times with 50 mL EA. The combined organic extracts were dried over sodium sulphate, filtered, and concentrated to dryness. The residue was purified by prep-HPLC, 15 to 35% MeCN/water with 0.1% FA modifier to afford (1R,3S)-3-(3-(2-(2-formyl-3-hydroxy-5-methoxyphenoxy)acetamido)-1H-pyrazol-5-yl)cyclopentyl (1-(difluoromethyl)cyclopropyl)carbamate (17 mg, 0.03 mmol, 7.1% yield) as a white solid. ¹H NMR (300 MHz, DMSO-D6) δ 12.31 (s, 1H), 12.16 (s, 1H), 10.54 (s, 1H), 10.13 (s, 1H), 7.80 (s, 1H), 6.30 (s, 1H), 6.13 (d, J=2.0 Hz, 1H), 6.05 (s, 1H), 5.99-5.45 (m, 1H), 4.99 (s, 1H), 4.79 (s, 2H), 3.79 (s, 4H), 3.03 (s, 1H), 2.02-1.76 (m, 2H), 1.62 (s, 3H), 0.93 (s, 2H), 0.81 (s, 2H). MS (ESI) calcd. for C₂₃H₂₆F₂N₄O₇, 508.18 m/z, found [M+H]⁺ 509.15 m/z.

### Example 4.39: Synthesis of Compound 172: (1R,3S)-3-(3-(2-(2-formyl-3-hydroxy-5-methoxyphenoxy)acetamido)-1H-pyrazol-5-yl)cyclopentyl 1,2,2-trimethylhydrazine-1-carboxylate

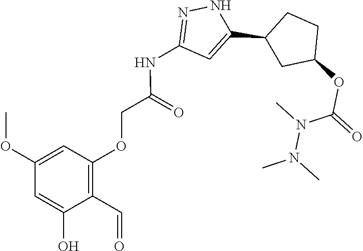 [SMILES: COc1cc(O)c(C=O)c(OCC(=O)Nc2cc([C@H]3CC[C@@H](OC(=O)N(C)N(C)C)C3)[nH]n2)c1]

### Synthetic Route

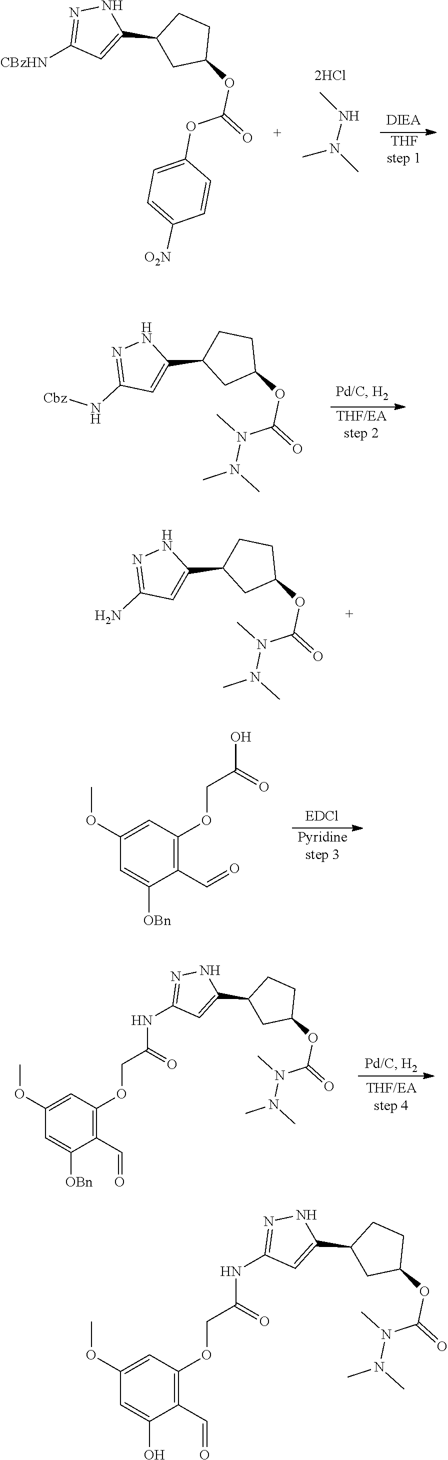

### Step 1: Synthesis of (1R,3S)-3-(3-(((benzyloxy)carbonyl)amino)-1H-pyrazol-5-yl)cyclopentyl 1,2,2-trimethylhydrazine-1-carboxylate

To a solution of intermediate C (1 g, 2.144 mmol, 1 equiv.) in THF (10 mL) was added 1,1,2-trimethylhydrazine dihydrochloride (630 mg, 4.288 mmol, 2 equiv.), DIEA (1.9 mL, 10.720 mmol, 5 equiv.). After stirring for 2 h at room temperature, the resulting mixture was diluted with EA (50 mL). The combined organic extracts were washed with 0.5 M NaOH (2×100 mL) and then with brine (2×100 mL), dried over anhydrous sodium sulfate. After filtration, the filtrate was concentrated under reduced pressure. The crude product was purified by reverse phase, 10 to 50% MeCN/water to afford benzyl N-{5-[(1S,3R)-3-[(N,N′,N′-trimethylhydrazinecarbonyl)oxy]cyclopentyl]-1H-pyrazol-3-yl}carbamate (300 mg, 29% yield) as a yellow solid. MS (ESI) calcd. for C₂₀H₂₇N₅O₄, 401.21 m/z, found [M+H]⁺ 402.10 m/z.

### Step 2: Synthesis of (1R,3S)-3-(3-amino-1H-pyrazol-5-yl)cyclopentyl 1,2,2-trimethylhydrazine-1-carboxylate

To a solution of benzyl N-{5-[(1S,3R)-3-[(N,N′,N′-trimethylhydrazinecarbonyl)oxy]cyclopentyl]-1H-pyrazol-3-yl}carbamate (270 mg, 0.673 mmol, 1 equiv.) in EA (3 mL) and THF (3 mL) was added 10% Pd/C (358 mg). After stirring for 2 h at room temperature under H₂ atmosphere, the resulting mixture was filtered, the filter cake was washed with DCM (3×10 mL), the filtrate was concentrated under reduced pressure to afford 1-{[(1R,3S)-3-(5-amino-2H-pyrazol-3-yl)cyclopentyl]oxy}-N,N′,N′-trimethylformohydrazide (150 mg, 66% yield). MS (ESI) calcd. for C₁₂H₂₁N₅O₂, 267.17 m/z, found [M+H]⁺ 268.10 m/z. The crude material was used without any further purification.

### Step 3: Synthesis of (1R,3S)-3-(3-(2-(3-(benzyloxy)-2-formyl-5-methoxyphenoxy)acetamido)-1H-pyrazol-5-yl)cyclopentyl 1,2,2-trimethylhydrazine-1-carboxylate

To a solution of 1-{[(1R,3S)-3-(5-amino-2H-pyrazol-3-yl)cyclopentyl]oxy}-N,N′,N′-trimethylformohydrazide (150 mg, 0.561 mmol, 1 equiv.) in pyridine (5 mL) was added intermediate B (213 mg, 0.673 mmol, 1.2 equiv.), EDCI (161 mg, 0.842 mmol, 1.5 equiv.). After stirring for 1 h at room temperature, the resulting mixture was diluted with water and extracted with EA (3×30 mL). The combined organic extracts were washed with brine (50 mL), dried over anhydrous sodium sulfate. After filtration, the filtrate was concentrated under reduced pressure. The crude product was purified by reverse phase, 10 to 50% MeCN/water to afford 2-[3-(benzyloxy)-2-formyl-5-methoxyphenoxy]-N-{5-[(1S,3R)-3-[(N,N′,N′-trimethylhydrazinecarbonyl)oxy]cyclopentyl]-1H-pyrazol-3-yl}acetamide (70 mg, 17% yield) as a white solid. MS (ESI) calcd. for C₂₉H₃₅N₅O₇, 565.25 m/z, found [M+H]⁺ 566.30 m/z.

### Step 4: Synthesis of (1R,3S)-3-(3-(2-(2-formyl-3-hydroxy-5-methoxyphenoxy)acetamido)-1H-pyrazol-5-yl)cyclopentyl 1,2,2-trimethylhydrazine-1-carboxylate

To a solution of 2-[3-(benzyloxy)-2-formyl-5-methoxyphenoxy]-N-{5-[(1S,3R)-3-[(N,N′,N′-trimethylhydrazinecarbonyl)oxy]cyclopentyl]-1H-pyrazol-3-yl}acetamide (60 mg, 0.106 mmol, 1 equiv.) in EA (1 mL) and THF (1 mL) was added 10% Pd/C (56 mg). After stirring for 2 h at room temperature under H₂ atmosphere, the resulting mixture was filtered, the filter cake was washed with DCM (3×10 mL), the filtrate was concentrated under reduced pressure. The crude was purified by prep-HPLC, 10 to 50% MeCN/water with 0.05% TFA modifier to afford 2-(2-formyl-3-hydroxy-5-methoxyphenoxy)-N-{5-[(1S,3R)-3-[(N,N′,N′-trimethylhydrazinecarbonyl)oxy]cyclopentyl]-1H-pyrazol-3-yl}acetamide (21.3 mg, 41% yield) as a white solid. MS (ESI) calcd. for C₂₂H₂₉N₅O₇, 475.21 m/z, found [M+H]⁺ 476.30 m/z. ¹H NMR (400 MHz, DMSO-D6) δ 12.33 (s, 1H), 10.56 (s, 1H), 10.15 (s, 1H), 6.34 (s, 1H), 6.15 (d, J=2.0 Hz, 1H), 6.07 (d, J=2.0 Hz, 1H), 5.06 (s, 1H), 4.81 (s, 2H), 3.81 (s, 3H), 3.10-3.20 (m, 1H), 2.83 (s, 3H), 2.44-2.50 (m, 6H), 2.36-2.43 (m, 1H), 2.00-2.07 (m, 1H), 1.70-1.89 (m, 4H).

### Example 4.40: Synthesis of Compound 182: (1s,3s)-3-(3-(2-(2-formyl-3-hydroxy-5-methoxyphenoxy)acetamido)-1H-pyrazol-5-yl)cyclobutyl propylcarbamate

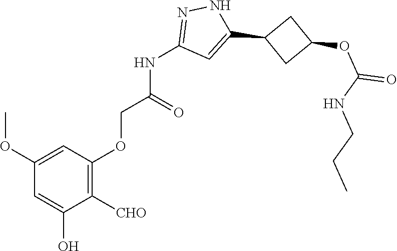 [SMILES: CCCNC(=O)O[C@H]1C[C@@H](c2cc(NC(=O)COc3cc(OC)cc(O)c3C=O)n[nH]2)C1]

### Synthetic Route

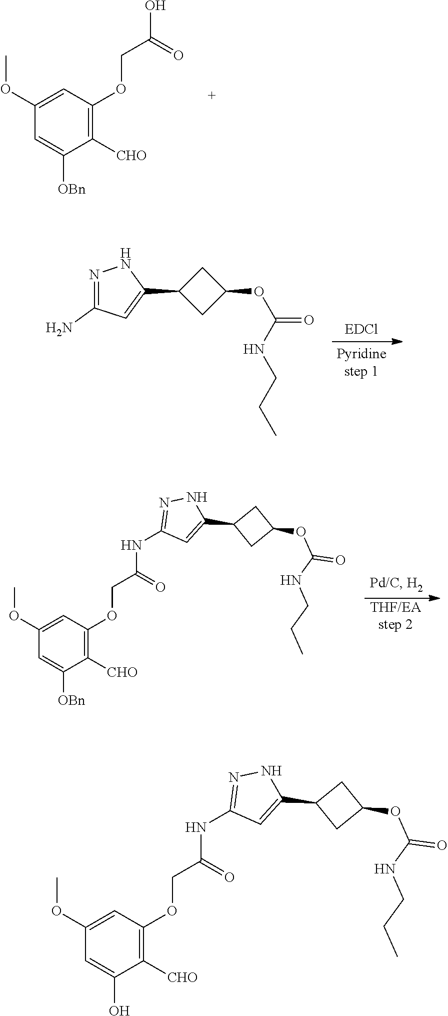 [SMILES: COc1cc(OCC(=O)O)c(C=O)c(OCc2ccccc2)c1 ; CCCNC(=O)O[C@H]1C[C@@H](c2cc(N)n[nH]2)C1 ; CCCNC(=O)O[C@H]1C[C@@H](c2cc(NC(=O)COc3cc(OC)cc(OCc4ccccc4)c3C=O)n[nH]2)C1 ; "THF/EAstep 2" ; CCCNC(=O)O[C@H]1C[C@@H](c2cc(NC(=O)COc3cc(OC)cc(O)c3C=O)n[nH]2)C1]

### Step 1: Synthesis of (1s,3s)-3-(3-(2-(3-(benzyloxy)-2-formyl-5-methoxyphenoxy)acetamido)-1H-pyrazol-5-yl)cyclobutyl propylcarbamate

Into a 20 mL vial were added intermediate B (207.0 mg, 0.65 mmol, 1.2 equiv), (1s,3s)-3-(3-amino-1H-pyrazol-5-yl)cyclobutyl propylcarbamate (130.0 mg, 0.54 mmol, 1.0 equiv) EDCI (156.8 mg, 0.82 mmol, 1.5 equiv) and pyridine (5 mL), the mixture was stirred at r.t overnight, the reaction was checked by LCMS and showed complete consumption of starting material. The mixture was purified by reverse phase preparatory HPLC, 10-50% ACN/water with 0.1% FA modifier to afford (1s,3s)-3-(3-(2-(3-(benzyloxy)-2-formyl-5-methoxyphenoxy)acetamido)-1H-pyrazol-5-yl)cyclobutyl propylcarbamate (112.0 mg, 30.61% yield) as a yellow solid. MS (ESI) calcd. for C₂₈H₃₂N₄O₇, 536.23 m/z, found [M+H]⁺ 537.05 m/z. (1s,3s)-3-(3-amino-1H-pyrazol-5-yl)cyclobutyl propylcarbamate was synthesized analogously as in compound 33.

### Step 2: Synthesis of (1s,3s)-3-(3-(2-(2-formyl-3-hydroxy-5-methoxyphenoxy)acetamido)-1H-pyrazol-5-yl)cyclobutyl propylcarbamate

A solution of (1s,3s)-3-(3-amino-1H-pyrazol-5-yl) cyclobutyl propylcarbamate (80.0 mg, 0.14 mmol, 1.0 equiv) and Pd/C (79.92 mg, 10% on active carbon) in 1 mL THF and 1 mL EA was stirred at r.t for 1 h under H₂ atmosphere, the reaction was checked by LCMS and showed complete consumption of starting material. The resulting mixture was filtered, the filter cake was washed with 20 mL EA. The filtrate was concentrated under reduced pressure. The residue was purified by prep-HPLC, 10 to 60% ACN/water with 0.1% FA modifier to afford (1s,3s)-3-(3-(2-(2-formyl-3-hydroxy-5-methoxyphenoxy)acetamido)-1H-pyrazol-5-yl)cyclobutyl propylcarbamate (6.9 mg, 10.33% yield) as a white solid. ¹H NMR (400 MHz, DMSO-D6) δ 12.32 (s, 1H), 12.26 (s, 1H), 10.57 (s, 1H), 10.14 (s, 1H), 7.16 (t, J=5.8 Hz, 1H), 6.34 (s, 1H), 6.14-6.06 (m, 2H), 4.85-4.73 (m, 3H), 3.80 (s, 3H), 3.05 (t, J=8.6 Hz, 1H), 2.90 (q, J=6.6 Hz, 2H), 2.65 (q, J=9.2 Hz, 2H), 2.10-1.98 (m, 2H), 1.45-1.38 (m, 2H), 0.81 (t, J=7.4 Hz, 3H). MS (ESI) calcd. for C₂₁H₂₆N₄O₇, 446.18 m/z, found [M+H]⁺ 447.25 m/z.

### Example 4.41: Synthesis of Compound 207: (1R,3S)-3-{5-[2-(2-formyl-3-hydroxy-5-methoxyphenoxy)acetamido]-2H-pyrazol-3-yl}cyclopentyl 3-oxa-8-azabicyclo[3.2.1]octane-8-carboxylate

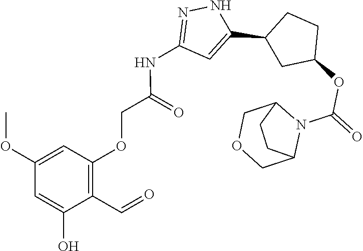 [SMILES: COc1cc(O)c(C=O)c(OCC(=O)Nc2cc([C@H]3CC[C@@H](OC(=O)N4C5CCC4COC5)C3)[nH]n2)c1]

### Synthetic Route

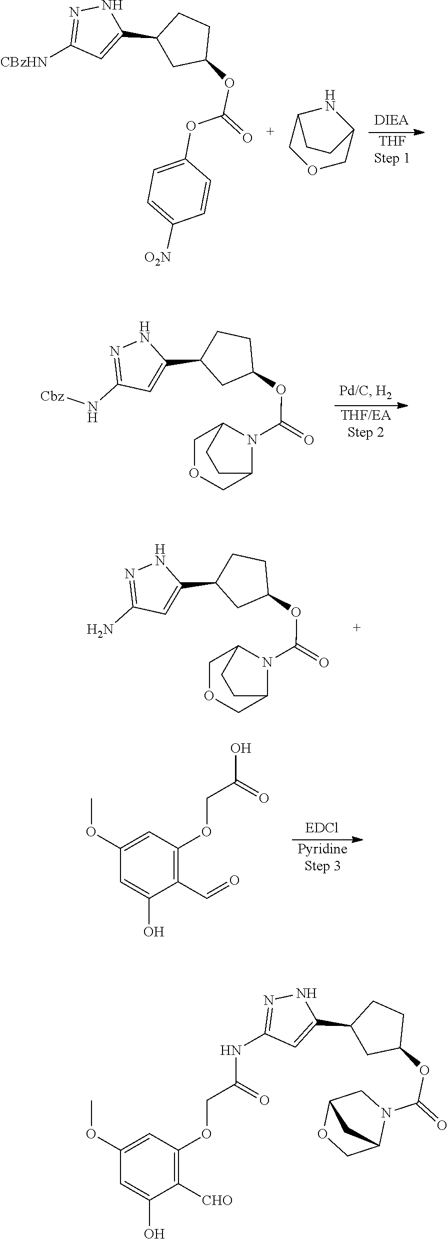

### Step 1: (1R,3S)-3-(3-(((benzyloxy)carbonyl)amino)-1H-pyrazol-5-yl)cyclopentyl 3-oxa-8-azabicyclo[3.2.1]octane-8-carboxylate

A 100 mL flask was charged with intermediate C (2.0 g, 4.3 mmol, 1.0 equiv), 3-oxa-8-azabicyclo[3.2.1]octane hydrochloride (1.9 g, 12.9 mmol, 3.0 equiv), 20 mL THF and DIEA (1.7 g, 12.9 mmol, 3.0 equiv). The resulting mixture was stirred at r.t for 1 h. The reaction mixture was quenched with 50 mL water. The organics were separated, and the aqueous phase was extracted 3 times with 50 mL EA. The combined organic extracts were dried over sodium sulphate, filtered, and concentrated to dryness. The crude material was purified by prep-HPLC, 48 to 50% MeCN/water with 0.05% FA modifier to afford (1R,3S)-3-(3-(((benzyloxy)carbonyl)amino)-1H-pyrazol-5-yl)cyclopentyl 3-oxa-8-azabicyclo[3.2.1]octane-8-carboxylate (1.1 g, 2.5 mmol, 79.0% yield) as a white solid. MS (ESI) calcd. for C₂₃H₂₈N₄O₅, 440.21 m/z, found [M+H]⁺ 441.20 m/z.

### Step 2: (1R,3S)-3-(3-amino-1H-pyrazol-5-yl)cyclopentyl 3-oxa-8-azabicyclo[3.2.1]octane-8-carboxylate

An oven dried 50 mL flask equipped with a stir bar under an atmosphere of hydrogen was charged with (1R,3S)-3-(3-(((benzyloxy)carbonyl)amino)-1H-pyrazol-5-yl)cyclopentyl 3-oxa-8-azabicyclo[3.2.1]octane-8-carboxylate (1.1 g, 2.5 mmol, 1.0 equiv), 10 mL EA, 10 mL THF and Pd/C (1.3 g, 1.2 mmol, 0.5 equiv, 10% on active carbon). The resulting mixture was stirred at r.t for 1 h. The resulting mixture was filtered. The filter cake was washed with 10 mL THF. The filtrate was concentrated to dryness to afford (1R,3S)-3-(3-amino-1H-pyrazol-5-yl)cyclopentyl 3-oxa-8-azabicyclo[3.2.1]octane-8-carboxylate (800.0 mg, 2.6 mmol, 99.2% yield) as an orange solid, the crude material was used for next step without any further purification. MS (ESI) calcd. for C₁₅H₂₂N₄O₃, 306.17 m/z, found [M+H]⁺ 307.15 m/z.

### Step 3: (1R,3S)-3-(3-(2-(2-formyl-3-hydroxy-5-methoxyphenoxy)acetamido)-1H-pyrazol-5-yl)cyclopentyl 3-oxa-8-azabicyclo[3.2.1]octane-8-carboxylate

A 15 mL vial was charged with (1R,3S)-3-(3-amino-1H-pyrazol-5-yl)cyclopentyl 3-oxa-8-azabicyclo[3.2.1]octane-8-carboxylate (800.0 mg, 2.6 mmol, 1.0 equiv), intermediate A (590.6 mg, 2.6 mmol, 1.0 equiv), EDCI (750.9 mg, 3.9 mmol, 1.5 equiv) and 8 mL pyridine. The resulting mixture was stirred at r.t for 1 h. The reaction mixture was diluted with 50 mL water and extracted 3 times with 50 mL EA. The combined organic extracts were dried over sodium sulphate, filtered and concentrated to dryness. The mixture was purified by prep-HPLC, 15 to 35% MeCN/water with 0.05% TFA modifier to afford (1R,3S)-3-(3-(2-(2-formyl-3-hydroxy-5-methoxyphenoxy)acetamido)-1H-pyrazol-5-yl)cyclopentyl 3-oxa-8-azabicyclo[3.2.1]octane-8-carboxylate (450.0 mg, 0.9 mmol, 27.3% yield) as a white solid. ¹H NMR (400 MHz, DMSO-D6) δ 12.32 (s, 1H), 10.56 (s, 1H), 10.14 (s, 1H), 6.32 (s, 1H), 6.14 (d, J=2.1 Hz, 1H), 6.07 (d, J=2.1 Hz, 1H), 5.07 (d, J=4.6 Hz, 1H), 4.81 (s, 2H), 3.98 (s, 2H), 3.80 (s, 3H), 3.59-3.28 (m, 4H), 3.21-2.99 (m, 1H), 2.39 (s, 1H), 2.01 (t, J=8.2 Hz, 1H), 1.91 (s, 1H), 1.76 (s, 7H). MS (ESI) calcd. for C₂₅H₃₀N₄O₈, 514.21 m/z, found [M+H]⁺ 515.15 m/z.

### Example 4.42: Synthesis of Compound 211: (1R,3S)-3-(3-(2-(5-ethoxy-2-formyl-3-hydroxyphenoxy)acetamido)-1H-pyrazol-5-yl)cyclopentyl 2,2-dimethylazetidine-1-carboxylate

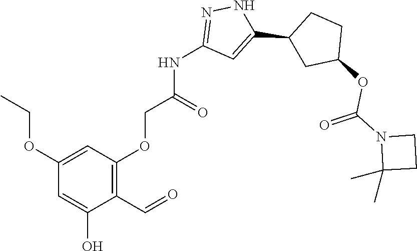 [SMILES: CCOc1cc(O)c(C=O)c(OCC(=O)Nc2cc([C@H]3CC[C@@H](OC(=O)N4CCC4(C)C)C3)[nH]n2)c1]

### Synthetic Route

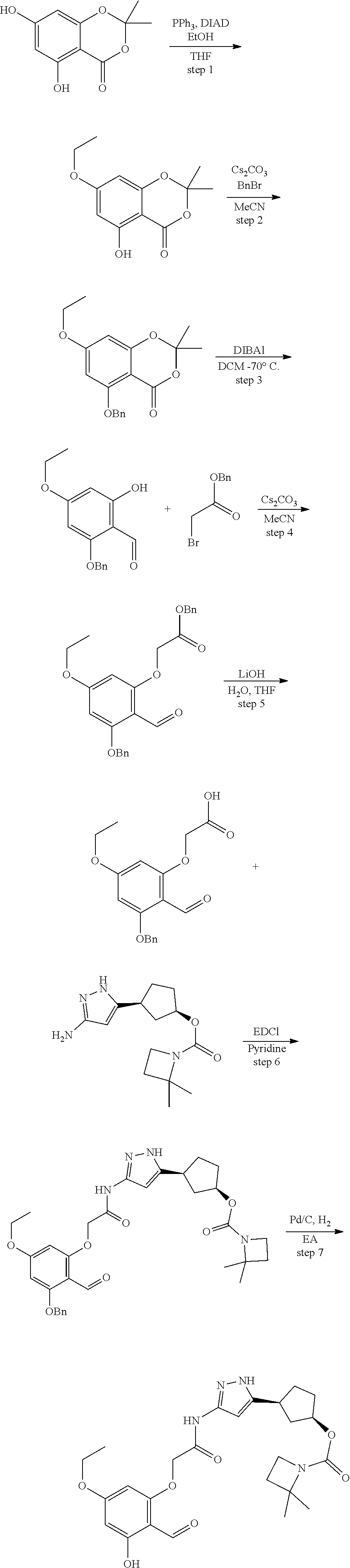

### Step 1: Synthesis of 7-ethoxy-5-hydroxy-2,2-dimethyl-4H-benzo[d][1,3]dioxin-4-one

5,7-dihydroxy-2,2-dimethyl-4H-benzo[d][1,3]dioxin-4-one (12 g, 57.093 mmol, 1 equiv.), PPh₃ (16.47 g, 62.802 mmol, 1.1 equiv.), EtOH (2.89 g, 62.802 mmol, 1.1 equiv.), a stir bar and THF (150 mL) were added to a nitrogen-purged 500 mL round-bottom flask and stirred until homogenous, then DIAD (12.7 g, 62.802 mmol, 1.1 equiv.) was added dropwise at 0° C. The resulting mixture was stirred for 2 h at rt under nitrogen atmosphere. The reaction was checked by LCMS and showed complete consumption of starting material. The crude reaction was quenched with 100 mL of water. The resulting mixture was extracted three times with 200 mL EA. The combined organic extracts were washed with brine, dried over anhydrous sodium sulfate, filtered, and concentrated in vacuo. The residue was purified by silica gel chromatography, 0 to 15% EA/PE to afford 7-ethoxy-5-hydroxy-2,2-dimethyl-4H-benzo[d][1,3]dioxin-4-one (10.7 g, 78% yield).

### Step 2: Synthesis of 5-(benzyloxy)-7-ethoxy-2,2-dimethyl-4H-benzo[d][1,3]dioxin-4-one

7-ethoxy-5-hydroxy-2,2-dimethyl-4H-benzo[d][1,3]dioxin-4-one (4 g, 16.790 mmol, 1 equiv.), Cs₂CO₃ (10.94 g, 33.580 mmol, 2 equiv.), BnBr (4.31 g, 25.185 mmol, 1.5 equiv.), a stir bar and MeCN (50 mL) was added to an oven-dried 250 mL round-bottom flask and stirred until homogenous. The resulting mixture was stirred overnight at rt. The reaction was checked by LCMS and showed complete consumption of starting material. The crude reaction was quenched with 40 mL of water. The resulting mixture was extracted three times with 70 mL EA. The combined organic extracts were washed with brine, dried over anhydrous sodium sulfate, filtered, and concentrated in vacuo. The residue was purified by silica gel chromatography, 0 to 20% EA/PE to afford 5-(benzyloxy)-7-ethoxy-2,2-dimethyl-4H-benzo[d][1,3]dioxin-4-one (5 g, 90% yield).

### Step 3: Synthesis of 2-(benzyloxy)-4-ethoxy-6-hydroxybenzaldehyde

5-(benzyloxy)-7-ethoxy-2,2-dimethyl-4H-benzo[d][1,3]dioxin-4-one (7 g, 21.318 mmol, 1 equiv.), a stir bar and DCM (80 mL) were added to a nitrogen-purged 250 mL round-bottom flask and stirred until homogenous, then DIBAl 1.5 mol/L solution in toluene (25.6 mL, 38.372 mmol, 1.8 equiv) was added dropwise at −75° C. The resulting mixture was stirred for 4 h at −75° C. under nitrogen atmosphere. The reaction was checked by LCMS and showed complete consumption of starting material. The crude reaction was quenched with 20 mL of MeOH at 0° C. The pH of the mixture was adjusted to around 1 with 2 N HCl. The resulting mixture was extracted three times with 70 mL EA. The combined organic extracts were washed with brine, dried over anhydrous sodium sulfate, filtered, and concentrated in vacuo. The residue was purified by silica gel chromatography, 0 to 30% EA/PE to afford 2-(benzyloxy)-4-ethoxy-6-hydroxybenzaldehyde (1.9 g, 32% yield).

### Step 4: Synthesis of benzyl 2-(3-(benzyloxy)-5-ethoxy-2-formylphenoxy)acetate

2-(benzyloxy)-4-ethoxy-6-hydroxybenzaldehyde (1.8 g, 6.610 mmol, 1 equiv.), benzyl 2-bromoacetate (2.27 g, 9.915 mmol, 1.5 equiv.), Cs₂CO₃ (5.38 g, 16.525 mmol, 2.5 equiv.), a stir bar and MeCN (30 mL) were added to an oven-dried 100 mL round-bottom flask and stirred until homogenous. The resulting mixture was stirred for 2 h at rt. The reaction was checked by LCMS and showed complete consumption of starting material. The crude reaction was quenched with 15 mL of water. The resulting mixture was extracted three times with 40 mL EA. The combined organic extracts were washed with brine, dried over anhydrous sodium sulfate, filtered, and concentrated in vacuo. The residue was purified by silica gel chromatography, 0 to 40% EA/PE to afford benzyl 2-(3-(benzyloxy)-5-ethoxy-2-formylphenoxy)acetate (2.37 g, 85% yield).

### Step 5: Synthesis of 2-(3-(benzyloxy)-5-ethoxy-2-formylphenoxy)acetic acid

Benzyl 2-(3-(benzyloxy)-5-ethoxy-2-formylphenoxy)acetate (2.36 g, 5.613 mmol, 1 equiv.), LiOH (0.67 g, 28.065 mmol, 5 equiv.), water (15 mL), a stir bar and THF (15 mL) were added to an oven-dried 100 mL round-bottom flask and stirred until homogenous. The resulting mixture was stirred for 2 h at rt. The reaction was checked by LCMS and showed complete consumption of starting material. The crude reaction was quenched with 15 mL of water. The pH of the mixture was adjusted to around 5 with 2 N HCl. The resulting mixture was extracted three times with 25 mL EA. The combined organic extracts were washed with brine, dried over anhydrous sodium sulfate, filtered and concentrated in vacuo to afford 2-(3-(benzyloxy)-5-ethoxy-2-formylphenoxy)acetic acid (1.7 g, 91% yield). The crude material was used without any further purification.

### Step 6: Synthesis of (1R,3S)-3-(3-(2-(3-(benzyloxy)-5-ethoxy-2-formylphenoxy)acetamido)-1H-pyrazol-5-yl)cyclopentyl 2,2-dimethylazetidine-1-carboxylate

2-(3-(benzyloxy)-5-ethoxy-2-formylphenoxy)acetic acid (150 mg, 0.454 mmol, 1 equiv.), (1R,3S)-3-(3-amino-1H-pyrazol-5-yl)cyclopentyl 2,2-dimethylazetidine-1-carboxylate (151 mg, 0.545 mmol, 1.2 equiv.), EDCI (174 mg, 0.908 mmol, 2 equiv.), a stir bar and pyridine (5 mL) were added to an oven-dried 50 mL round-bottom flask and stirred until homogenous. The resulting mixture was stirred for 2 h at rt. The reaction was checked by LCMS and showed complete consumption of starting material. The crude reaction was quenched with 3 mL of water. The resulting mixture was extracted three times with 10 mL EA. The combined organic extracts were washed with brine, dried over anhydrous sodium sulfate, filtered, and concentrated in vacuo. The residue was purified by reverse-phase chromatography, 5 to 45% ACN/Water with 0.05% TFA modifier to afford (1R,3S)-3-(3-(2-(3-(benzyloxy)-5-ethoxy-2-formylphenoxy)acetamido)-1H-pyrazol-5-yl)cyclopentyl 2,2-dimethylazetidine-1-carboxylate (60 mg, 22% yield). (1R,3S)-3-(3-amino-1H-pyrazol-5-yl)cyclopentyl 2,2-dimethylazetidine-1-carboxylate was synthesized analogously as in Compound 162.

### Step 7: Synthesis of (1R,3S)-3-(3-(2-(5-ethoxy-2-formyl-3-hydroxyphenoxy)acetamido)-1H-pyrazol-5-yl)cyclopentyl 2,2-dimethylazetidine-1-carboxylate

(1R,3S)-3-(3-(2-(3-(benzyloxy)-5-ethoxy-2-formylphenoxy)acetamido)-1H-pyrazol-5-yl)cyclopentyl 2,2-dimethylazetidine-1-carboxylate (60 mg, 0.102 mmol, 1 equiv.), 10% Pd/C (12 mg), a stir bar and EA (5 mL) were added to a nitrogen-purged 50 mL round-bottom flask and stirred until homogenous. The resulting mixture was stirred overnight at rt under H₂ (1 atm). The reaction was checked by LCMS and showed complete consumption of starting material. The crude reaction was filtered and washed with EA (30 mL). The filtrate was concentrated in vacuo. The residue was purified by reverse-phase chromatography, 5 to 35% ACN/Water with 0.05% TFA modifier to afford (1R,3S)-3-(3-(2-(5-ethoxy-2-formyl-3-hydroxyphenoxy)acetamido)-1H-pyrazol-5-yl)cyclopentyl 2,2-dimethylazetidine-1-carboxylate (6.9 mg, 13% yield) as a white solid. MS (ESI) calcd. for C₂₅H₃₂N₄O₇ 500.23, found [M+H]⁺ 501.25 m/z. ¹H NMR (400 MHz, DMSO-D6) δ 12.31 (s, 1H), 12.16 (s, 1H), 10.52 (s, 1H), 10.14 (s, 1H), 6.32 (s, 1H), 6.11 (d, J=2.0 Hz, 1H), 6.04 (s, 1H), 4.99 (d, J=20.4 Hz, 1H), 4.80 (s, 2H), 4.15-4.04 (m, 2H), 3.78-3.57 (m, 2H), 3.15-3.00 (m, 1H), 2.47-2.29 (m, 1H), 2.08-1.97 (m, 1H), 1.96-1.79 (m, 3H), 1.79-1.57 (m, 3H), 1.40-1.25 (m, 9H).

### Example 4.43: Synthesis of Compound 231: (1R,3S)-3-{5-[2-(2-formyl-3-hydroxy-5-methoxyphenoxy)acetamido]-2H-pyrazol-3-yl}cyclopentyl 3-oxa-6-azabicyclo[3.1.1]heptane-6-carboxylate

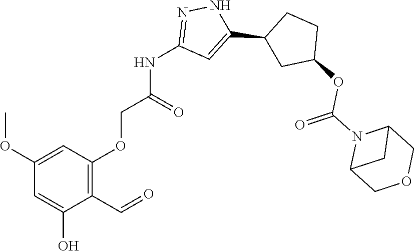 [SMILES: COc1cc(O)c(C=O)c(OCC(=O)Nc2cc([C@H]3CC[C@@H](OC(=O)N4C5COCC4C5)C3)[nH]n2)c1]

### Synthetic Route

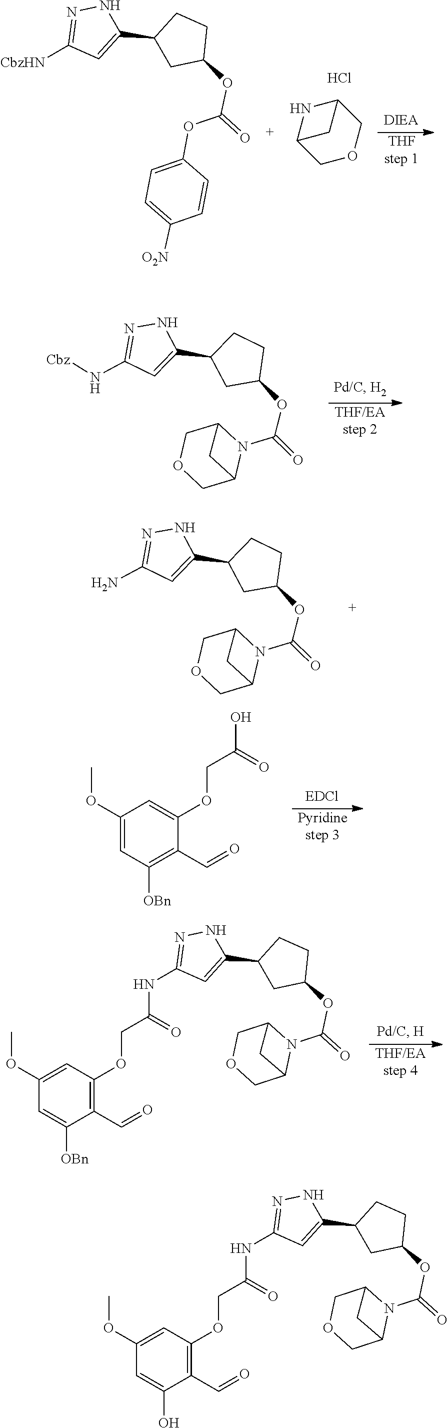

### Step 1: Synthesis of (1R,3S)-3-(5-{[(benzyloxy)carbonyl]amino}-2H-pyrazol-3-yl)cyclopentyl 3-oxa-6-azabicyclo[3.1.1]heptane-6-carboxylate

To a stirred solution of intermediate C (950.0 mg, 2.037 mmol, 1 equiv.) and 3-oxa-6-azabicyclo[3.1.1]heptane hydrochloride (828.5 mg, 6.111 mmol, 3 equiv.) in THF (20 mL) was added DIEA (3.5 mL, 20.370 mmol, 10 equiv.) dropwise at room temperature. The resulting mixture was stirred for 2 h at room temperature. The reaction was monitored by LCMS. The reaction was diluted with EA (60 mL), washed with 0.5 M NaOH (2×60 mL), and then with brine (2×60 mL). The combined organic extracts dried over anhydrous Na₂SO₄ and concentrated under reduced pressure. The crude material was purified by C18 reverse phase chromatography, 5-50% MeCN/H2O to afford (1R,3S)-3-(5-{[(benzyloxy)carbonyl]amino}-2H-pyrazol-3-yl)cyclopentyl 3-oxa-6-azabicyclo[3.1.1]heptane-6-carboxylate (280.0 mg, 29.11% yield) as a white solid. MS (ESI) calcd. for C₂₂H₂₆N₄O₅ 426.19 m/z, found [M+H]⁺ 427.10 m/z.

### Step 2: Synthesis of (1R,3S)-3-(3-amino-1H-pyrazol-5-yl)cyclopentyl 3-oxa-6-azabicyclo[3.1.1]heptane-6-carboxylate1

To a stirred solution of (1R,3S)-3-(5-{[(benzyloxy)carbonyl]amino}-2H-pyrazol-3-yl)cyclopentyl 3-oxa-6-azabicyclo[3.1.1]heptane-6-carboxylate (280.0 mg, 0.657 mmol, 1 equiv.) in THF (3 mL) and EA (3 mL) was added 10% Pd/C (200 mg). The mixture was stirred at room temperature for 2 h under hydrogen atmosphere. The reaction was monitored by LCMS. The resulting mixture was filtered, the filter cake was washed with DCM (2×10 mL). The filtrate was concentrated under reduced pressure resulting in (1R,3S)-3-(3-amino-1H-pyrazol-5-yl)cyclopentyl 3-oxa-6-azabicyclo[3.1.1]heptane-6-carboxylate (170.0 mg, 64.12% yield) as a yellow solid. The crude material was used without any further purification. MS (ESI) calcd. for C₁₄H₂₀N₄O₃ 292.15 m/z, found [M+H]⁺ 293.25 m/z.

### Step 3: Synthesis of (1R,3S)-3-(5-{2-[3-(benzyloxy)-2-formyl-5-methoxyphenoxy]acetamido}-2H-pyrazol-3-yl)cyclopentyl N-propylcarbamate

To a stirred solution of (1R,3S)-3-(3-amino-1H-pyrazol-5-yl)cyclopentyl 3-oxa-6-azabicyclo[3.1.1]heptane-6-carboxylate (160.0 mg, 0.547 mmol, 1 equiv.) and intermediate B (173.1 mg, 0.547 mmol, 1 equiv.) in pyridine (4 mL) was added EDCI (157.4 mg, 0.821 mmol, 1.5 equiv.) at room temperature. The resulting mixture was stirred for 1 h at room temperature. The reaction was monitored by LCMS. The reaction was quenched with water and the mixture was extracted with EA (2×10 mL). The combined organic extracts were washed with brine (2×10 mL), dried over anhydrous sodium sulfate, and concentrated in vacuo. The crude material was purified by C18 reverse phase chromatography, 5 to 70% MeCN/water to afford (1R,3S)-3-(5-{2-[3-(benzyloxy)-2-formyl-5-methoxyphenoxy]acetamido}-2H-pyrazol-3-yl)cyclopentyl (1R,4R)-2-oxa-5-azabicyclo[2.2.1]heptane-5-carboxylate (50.0 mg, 12.89% yield) as a yellow solid. MS (ESI) calcd. for C₃₁H₃₄N₄O₈ 590.24 m/z, found [M+H]⁺ 591.15 m/z.

### Step 4: Synthesis of (1R,3S)-3-{5-[2-(2-formyl-3-hydroxy-5-methoxyphenoxy)acetamido]-2H-pyrazol-3-yl}cyclopentyl 3-oxa-6-azabicyclo[3.1.1]heptane-6-carboxylate

To a stirred solution of (1R,3S)-3-(5-{2-[3-(benzyloxy)-2-formyl-5-methoxyphenoxy]acetamido}-2H-pyrazol-3-yl)cyclopentyl (1R,4R)-2-oxa-5-azabicyclo[2.2.1]heptane-5-carboxylate (50.0 mg, 0.085 mmol, 1 equiv.) in THF (3 mL) and EA (3 mL) was added 10% Pd/C (50 mg). The mixture was stirred at room temperature for 4 h under hydrogen atmosphere. The reaction was monitored by LCMS. The resulting mixture was filtered, the filter cake was washed with DCM (2×10 mL). The filtrate was concentrated under reduced pressure. The crude material was purified by prep-HPLC, 5 to 44% MeCN/H₂O with 0.1% FA modifier to afford (1R,3S)-3-{5-[2-(2-formyl-3-hydroxy-5-methoxyphenoxy)acetamido]-2H-pyrazol-3-yl}cyclopentyl 3-oxa-6-azabicyclo[3.1.1]heptane-6-carboxylate (585.9 mg, 35.12% yield) as a white solid. ¹H NMR (400 MHz, DMSO-D6) δ 12.33 (s, 1H), 10.55 (s, 1H), 10.15 (s, 1H), 6.32 (s, 1H), 6.14-6.15 (m, 1H), 6.06-6.07 (m, 1H), 5.07-5.12 (m, 1H), 4.81 (s, 2H), 4.03-4.05 (m, 3H), 3.86-3.98 (m, 1H), 3.81 (s, 3H), 3.62-3.65 (m, 2H), 3.10-3.14 (m, 1H), 2.43-2.48 (m, 2H), 2.00-2.08 (m, 1H), 1.88-1.95 (m, 1H), 1.67-1.77 (m, 3H), 1.56 (d, J=8.4 Hz, 1H). MS (ESI) calcd. for C₂₄H₂₈N₄O₈ 500.19 m/z, found [M+H]⁺ 501.25 m/z.

### Example 4.44: Synthesis of Compound 234: (1R,3S)-3-(3-(2-(2-formyl-3-hydroxy-5-methoxyphenoxy)acetamido)-1H-pyrazol-5-yl)cyclopentyl piperidine-1-carboxylate

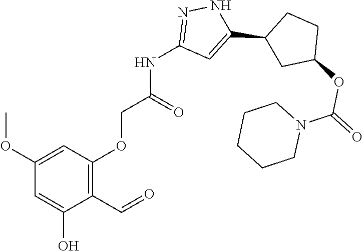 [SMILES: COc1cc(O)c(C=O)c(OCC(=O)Nc2cc([C@H]3CC[C@@H](OC(=O)N4CCCCC4)C3)[nH]n2)c1]

### Synthetic Route

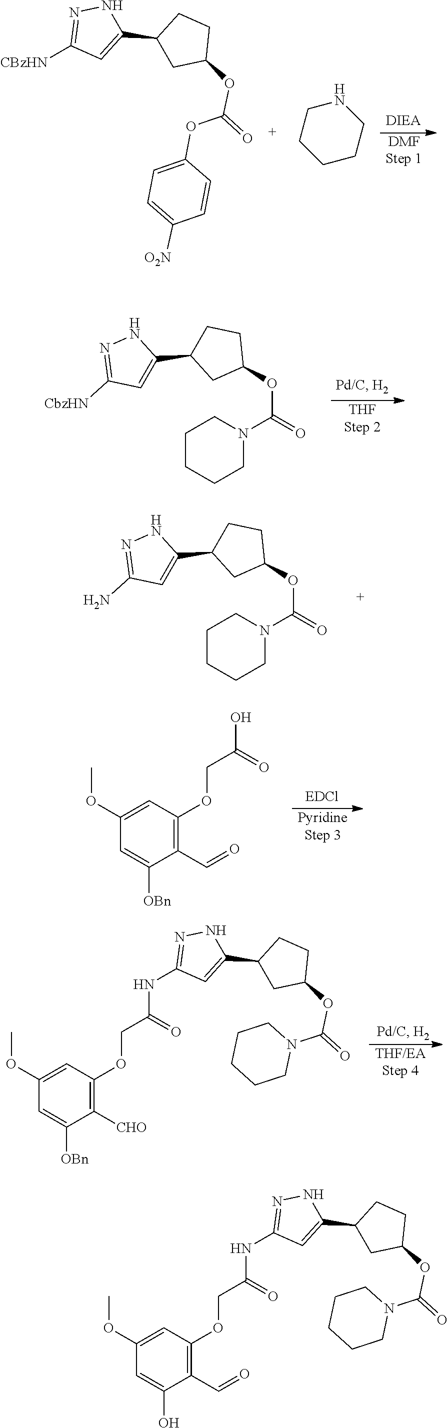

### Step 1: Synthesis of (1R,3S)-3-(3-(((benzyloxy)carbonyl)amino)-1H-pyrazol-5-yl)cyclopentyl piperidine-1-carboxylate

A solution of intermediate C (900.0 mg, 1.929 mmol, 1 equiv), piperidine (492.88 mg, 5.787 mmol, 3 equiv) and DIEA (2.35 mL, 13.503 mmol, 7 equiv) in THF (10 mL) was stirred for 2 h at r.t. The reaction was diluted with EA (60 mL), washed with 0.5 M NaOH (2×60 mL), and then with brine (2×60 mL). The combined organic extracts dried over anhydrous sodium sulfate and concentrated in vacuo. The crude material was diluted with DMF and purified by prep-HPLC, 10 to 70% MeCN/water with 0.05% TFA modifier to afford (1R,3S)-3-(5-{[(benzyloxy)carbonyl]amino}-2H-pyrazol-3-yl)cyclopentyl piperidine-1-carboxylate (550.0 mg, 69.11% yield) as orange oil.

### Step 2: Synthesis of (1R,3S)-3-(3-amino-1H-pyrazol-5-yl)cyclopentyl piperidine-1-carboxylate

A solution of (1R,3S)-3-(5-{[(benzyloxy)carbonyl]amino}-2H-pyrazol-3-yl)cyclopentyl piperidine-1-carboxylate (550.0 mg, 1.333 mmol, 1 equiv) and 10% Pd/C (550.56 mg) in THF (5 mL) and EA (5 mL) was stirred for 3 h at r.t under H₂ atmosphere. The resulting mixture was filtered, the filter cake was washed with EA (3×20 mL). The filtrate was concentrated under reduced pressure. The crude product (1R,3S)-3-(5-amino-2H-pyrazol-3-yl)cyclopentyl piperidine-1-carboxylate (410 mg, 77.33% yield) was used in the next step directly without further purification.

### Step 3: Synthesis of ((1R,3S)-3-(3-(2-(3-(benzyloxy)-2-formyl-5-methoxyphenoxy)acetamido)-1H-pyrazol-5-yl)cyclopentyl piperidine-1-carboxylate

A solution of (1R,3S)-3-(5-amino-2H-pyrazol-3-yl)cyclopentyl piperidine-1-carboxylate (100 mg, 0.359 mmol, 1 equiv), intermediate B (113.63 mg, 0.359 mmol, 1 equiv) and EDCI (103.30 mg, 0.538 mmol, 1.5 equiv) in pyridine (2 mL) was stirred for 3 h at r.t. The resulting mixture was diluted with 15 mL water and extracted with EA (3×10 mL). The combined organic extracts were washed with brine (3×15 mL) and dried over anhydrous sodium sulfate. After filtration, the filtrate was concentrated under reduced pressure. The crude material was diluted with DMF and purified by prep-HPLC, 10 to 70% MeCN/water with 0.05% TFA modifier to afford (1R,3S)-3-(5-{2-[3-(benzyloxy)-2-formyl-5-methoxyphenoxy]acetamido}-2H-pyrazol-3-yl)cyclopentyl piperidine-1-carboxylate (40 mg, 19.31% yield) as off-white solid.

### Step 4: Synthesis of (1R,3S)-3-(3-(2-(2-formyl-3-hydroxy-5-methoxyphenoxy)acetamido)-1H-pyrazol-5-yl)cyclopentyl piperidine-1-carboxylate

A solution of (1R,3S)-3-(5-{2-[3-(benzyloxy)-2-formyl-5-methoxyphenoxy]acetamido}-2H-pyrazol-3-yl)cyclopentyl piperidine-1-carboxylate (50 mg, 0.087 mmol, 1 equiv) and 10% Pd/C (50 mg) in THF (2 mL) and EA (2 mL) was stirred for 3 h at r.t. under H₂ atmosphere. The resulting mixture was filtered, the filter cake was washed with EA (4×15 mL). The filtrate was concentrated under reduced pressure. The crude material was diluted with DMF and purified by prep-HPLC, 10 to 70% MeCN/water with 0.05% TFA modifier to afford (1R,3S)-3-{5-[2-(2-formyl-3-hydroxy-5-methoxyphenoxy)acetamido]-2H-pyrazol-3-yl}cyclopentyl piperidine-1-carboxylate (9.5 mg, 22.13% yield) as white solid. MS (ESI) calcd. for C₂₄H₃₀N₄O₇, 486.21 m/z, found [M+H]⁺ 487.15 m/z. ¹H NMR (400 MHz, DMSO-D6) δ 12.32 (s, 1H), 10.55 (s, 1H), 10.14 (s, 1H), 6.31 (s, 1H), 6.14 (d, J=2 Hz, 1H), 6.06 (d, J=2.4 Hz, 1H), 4.98-5.03 (m, 1H), 4.80 (s, 2H), 3.80 (s, 3H), 3.24-3.27 (m, 4H), 3.07-3.13 (m, 1H), 2.35-2.42 (m, 1H), 1.98-2.05 (m, 1H), 1.83-1.92 (m, 1H), 1.65-1.78 (m, 3H), 1.45-1.51 (m, 2H), 1.35-1.37 (m, 4H).

### Example 4.45: Compound 239: (1R,3S)-3-(3-(2-(2-formyl-3-hydroxy-5-methoxyphenoxy)acetamido)-1H-pyrazol-5-yl)cyclopentyl 2,2-dimethylazetidine-1-carboxylate

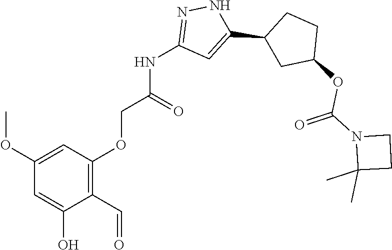 [SMILES: COc1cc(O)c(C=O)c(OCC(=O)Nc2cc([C@H]3CC[C@@H](OC(=O)N4CCC4(C)C)C3)[nH]n2)c1]

### Synthetic Route

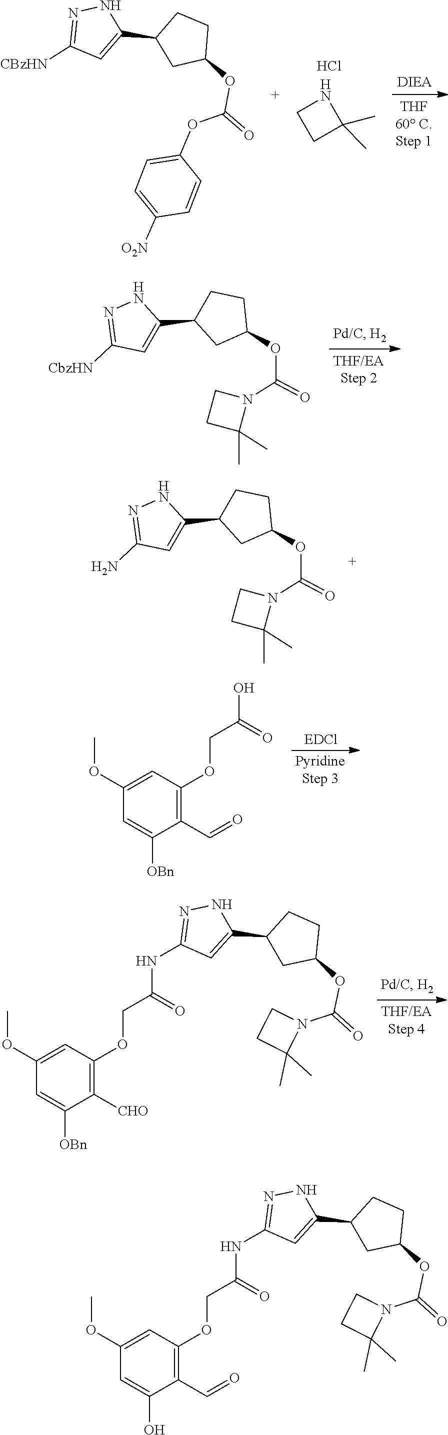

### Step 1: Synthesis of (1R,3S)-3-(5-{[(benzyloxy)carbonyl]amino}-2H-pyrazol-3-yl)cyclopentyl 2,2-dimethylazetidine-1-carboxylate

To a stirred solution of (1R,3S)-3-(5-{[(benzyloxy)carbonyl]amino}-2H-pyrazol-3-yl)cyclopentyl 4-nitrophenyl carbonate (0.90 g, 1.929 mmol, 1 equiv.) and 2,2-dimethylazetidine hydrochloride (0.35 g, 2.893 mmol, 1.5 equiv.) in THF (15 mL) was added DIEA (2.49 g, 19.290 mmol, 10 equiv.) at room temperature. The mixture was stirred at 60° C. for 2 h. The reaction was quenched with water (50 mL) at room temperature. The resulting mixture was extracted with EA (3×100 mL). The combined organic layers were washed with 0.5 M NaOH (3×100 mL) and then with brine (2×50 mL) and dried over anhydrous Na₂SO₄. After filtration, the filtrate was concentrated under reduced pressure. The residue was purified by C18 reverse phase chromatography, 50-60% MeCN/H₂O to afford (1R,3S)-3-(5-{[(benzyloxy)carbonyl]amino}-2H-pyrazol-3-yl)cyclopentyl 2,2-dimethylazetidine-1-carboxylate (0.47 g, 33.08% yield) as a light brown viscous oil. MS (ESI) calcd. for C₂₂H₂₈N₄O₄, 412.21 m/z, found [M+H]⁺ 413.15 m/z.

### Step 2: Synthesis of (1R,3S)-3-(5-amino-2H-pyrazol-3-yl)cyclopentyl 2,2-dimethylazetidine-1-carboxylate

To a stirred solution of (1R,3S)-3-(5-{[(benzyloxy)carbonyl]amino}-2H-pyrazol-3-yl)cyclopentyl 2,2-dimethylazetidine-1-carboxylate (450.0 mg, 1.091 mmol, 1 equiv.) in EA (10 mL) and THF (10 mL) was added 10% Pd/C (300.0 mg) in portions at room temperature. The mixture was stirred at room temperature under hydrogen atmosphere for 2 h. The resulting mixture was filtered, the filter cake was washed with DCM (3×50 mL). The filtrate was concentrated under reduced pressure to afford (1R,3S)-3-(5-amino-2H-pyrazol-3-yl)cyclopentyl 2,2-dimethylazetidine-1-carboxylate (280.0 mg, 85.01% yield) as a brown viscous oil. MS (ESI) calcd. for C₁₄H₂₂N₄O₂, 278.17 m/z, found [M+H]⁺ 279.10 m/z. The crude material was used without any further purification.

### Step 3: Synthesis of (1R,3S)-3-(5-{2-[3-(benzyloxy)-2-formyl-5-methoxyphenoxy]acetamido}-2H-pyrazol-3-yl)cyclopentyl 2,2-dimethylazetidine-1-carboxylate

To a stirred solution of (1R,3S)-3-(5-amino-2H-pyrazol-3-yl)cyclopentyl 2,2-dimethylazetidine-1-carboxylate (100.0 mg, 0.359 mmol, 1 equiv.) and 3-(benzyloxy)-2-formyl-5-methoxyphenoxyacetic acid (113.6 mg, 0.359 mmol, 1 equiv.) in pyridine (2 mL) was added EDCI (103.3 mg, 0.538 mmol, 1.5 equiv.) in portions at room temperature. The mixture was stirred at room temperature for 2 h. The reaction was quenched with water (20 mL) at room temperature. The resulting mixture was extracted with EA (3×30 mL). The combined organic layers were washed with brine (2×30 mL) and dried over anhydrous Na₂SO₄. After filtration, the filtrate was concentrated under reduced pressure. The residue was purified by C18 reverse phase chromatography, 50-60% MeCN/H₂O to afford (1R,3S)-3-(5-{2-[3-(benzyloxy)-2-formyl-5-methoxyphenoxy]acetamido}-2H-pyrazol-3-yl)cyclopentyl 2,2-dimethylazetidine-1-carboxylate (77.0 mg, 19.60% yield) as a light yellow solid. MS (ESI) calcd. for C₃₁H₃₆N₄O₇, 576.26 m/z, found [M+H]⁺ 577.30 m/z.

### Step 4: Synthesis of (1R,3S)-3-{5-[2-(2-formyl-3-hydroxy-5-methoxyphenoxy)acetamido]-2H-pyrazol-3-yl}cyclopentyl 2,2-dimethylazetidine-1-carboxylate

To a stirred solution of (1R,3S)-3-(5-{2-[3-(benzyloxy)-2-formyl-5-methoxyphenoxy]acetamido}-2H-pyrazol-3-yl)cyclopentyl 2,2-dimethylazetidine-1-carboxylate (70.0 mg, 0.121 mmol, 1 equiv.) in EA (3 mL) and THF (3 mL) was added 10% Pd/C (40.0 mg) in portions at room temperature. The mixture was stirred at room temperature under hydrogen atmosphere for 2 h. The resulting mixture was filtered, the filter cake was washed with DCM (3×20 mL). The filtrate was concentrated under reduced pressure. The residue was purified by C18 reverse phase chromatography, 50-60% MeCN/H₂O to afford (1R,3S)-3-{5-[2-(2-formyl-3-hydroxy-5-methoxyphenoxy)acetamido]-2H-pyrazol-3-yl}cyclopentyl 2,2-dimethylazetidine-1-carboxylate (5.1 mg, 8.45% yield) as a white solid. MS (ESI) calcd. for C₂₄H₃₀N₄O₇, 486.21 m/z, found [M+H]⁺ 487.10 m/z. 1H NMR (400 MHz, DMSO-D6) δ (ppm): 12.32 (br s, 1H), 12.18 (br s, 1H), 10.54 (s, 1H), 10.15 (s, 1H), 6.32 (s, 1H), 6.14 (d, J=1.6 Hz, 1H), 6.06 (d, J=2.0 Hz, 1H), 4.93-5.05 (m, 1H), 4.80 (s, 2H), 3.80 (s, 3H), 3.68-3.76 (m, 1H), 3.61-3.67 (m, 1H), 3.03-3.12 (m, 1H), 2.30-2.46 (m, 1H), 1.98-2.07 (m, 1H), 1.82-1.96 (m, 3H), 1.69-1.78 (m, 2H), 1.59-1.68 (m, 1H), 1.27-1.38 (m, 6H).

### Example 4.46: Synthesis of Compound 244: (1R,3S)-3-{5-[2-(2-formyl-3-hydroxy-5-methoxyphenoxy)acetamido]-2H-pyrazol-3-yl}cyclopentyl N-isopropyl-N-methylcarbamate

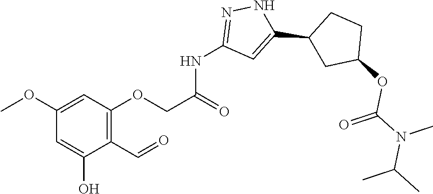

### Synthetic Route

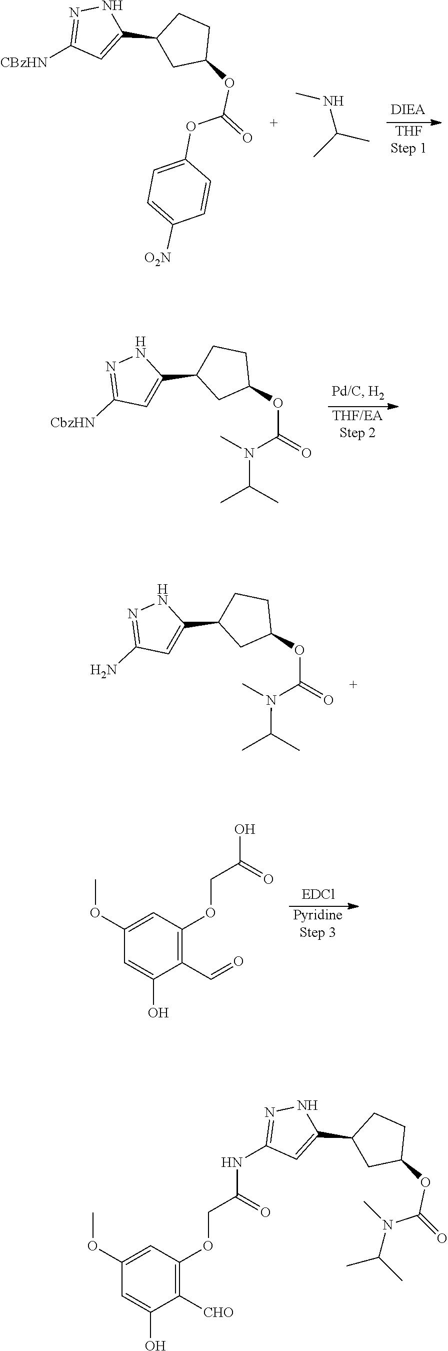

### Step 1: Synthesis of benzyl N-{5-[(1S,3R)-3-{[isopropyl(methyl)carbamoyl]oxy}cyclopentyl]-1H-pyrazol-3-yl}carbamate

To a stirred solution of intermediate C (6.5 g, 13.935 mmol, 1 equiv.) and N-methylpropan-2-amine (1.45 mL, 13.935 mmol, 1 equiv.) in THF (60 mL) was added DIEA (24.27 mL, 139.350 mmol, 10 equiv.) at 25° C. The resulting mixture was stirred overnight at 25° C. The reaction was diluted with EA (900 mL), washed with 0.5 M NaOH (3×400 mL), and then washed with brine (3×400 mL). The combined organic extracts were dried over anhydrous sodium sulfate, and concentrated in vacuo. The crude material was purified by C18 reverse phase chromatography, 5 to 60% MeCN/water to afford benzyl N-{5-[(1S,3R)-3-{[isopropyl(methyl)carbamoyl]oxy}cyclopentyl]-1H-pyrazol-3-yl}carbamate (3.6 g, 48.38% yield) as a yellow solid. MS (ESI) calcd. for C₂₁H₂₈N₄O₄, 400.21 m/z, found [M+H]⁺ 401.30 m/z.

### Step 2: Synthesis of (1R,3S)-3-(5-amino-2H-pyrazol-3-yl)cyclopentyl N-isopropyl-N-methylcarbamate

To a stirred solution of benzyl N-{5-[(1S,3R)-3-{[isopropyl(methyl)carbamoyl]oxy}cyclopentyl]-1H-pyrazol-3-yl}carbamate (3.6 g, 8.989 mmol, 1 equiv.) in THF (20 mL) and EA (20 mL) was added 10% Pd/C (3.6 g) at 25° C. The resulting mixture was stirred for 3 h at 25° C. under H₂ atmosphere. The resulting mixture was filtered, the filter cake was washed with DCM (4×100 mL). The filtrate was concentrated under reduced pressure. This resulted in (1R,3S)-3-(5-amino-2H-pyrazol-3-yl)cyclopentyl N-isopropyl-N-methylcarbamate (2.2 g, 82.70% yield) as a yellow oil. MS (ESI) calcd. for C₁₃H₂₂N₄O₂, 266.17 m/z, found [M+H]⁺ 267.30 m/z. The crude material was used without any further purification.

### Step 3: Synthesis of (1R,3S)-3-{5-[2-(2-formyl-3-hydroxy-5-methoxyphenoxy)acetamido]-2H-pyrazol-3-yl}cyclopentyl N-isopropyl-N-methylcarbamate

To a stirred solution of (1R,3S)-3-(5-amino-2H-pyrazol-3-yl)cyclopentyl N-isopropyl-N-methylcarbamate (600 mg, 2.253 mmol, 1 equiv.) and intermediate A (509.53 mg, 2.253 mmol, 1 equiv.) in pyridine (10 mL) was added EDCI (647.77 mg, 3.380 mmol, 1.5 equiv.) at 25° C. The resulting mixture was stirred for 3 h at 25° C. The reaction was quenched with water and then the mixture was extracted with EA (3×60 mL). The combined organic extracts were washed with brine (6×60 mL), dried over anhydrous sodium sulfate, and concentrated in vacuo. The crude material was purified by C18 reverse phase chromatography, 5-60% MeCN/water with 0.05% TFA to (1R,3S)-3-{5-[2-(2-formyl-3-hydroxy-5-methoxyphenoxy)acetamido]-2H-pyrazol-3-yl}cyclopentyl N-isopropyl-N-methylcarbamate (108.5 mg, 8.01% yield) as a white solid. MS (ESI) calcd. for C₂₃H₃₀N₄O₇, 474.21 m/z, found [M+H]⁺ 475.20 m/z. ¹H NMR (400 MHz, DMSO-D6) δ 12.32 (s, 2H), 10.55 (s, 1H), 10.15 (s, 1H), 6.32 (s, 1H), 6.14-6.15 (m, 1H), 6.06-6.07 (m, 1H), 5.00-5.02 (m, 1H), 4.80 (s, 2H), 4.00-4.09 (m, 1H), 3.81 (s, 3H), 3.09-3.13 (m, 1H), 2.62 (s, 3H), 2.38-2.40 (m, 1H), 2.01-2.05 (m, 1H), 1.90-1.91 (m, 1H), 1.65-1.78 (m, 3H), 0.99-1.01 (m, 6H).

### Example 4.47: Synthesis of Compound 249: (1R,3S)-3-{5-[2-(2-formyl-3-hydroxy-5-methoxyphenoxy)acetamido]-2H-pyrazol-3-yl}cyclopentyl N-{bicyclo[1.1.1]pentan-1-yl}carbamate

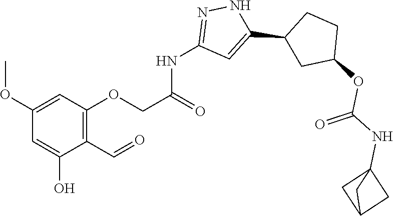 [SMILES: COc1cc(O)c(C=O)c(OCC(=O)Nc2cc([C@H]3CC[C@@H](OC(=O)NC45CC(C4)C5)C3)[nH]n2)c1]

### Synthetic Route

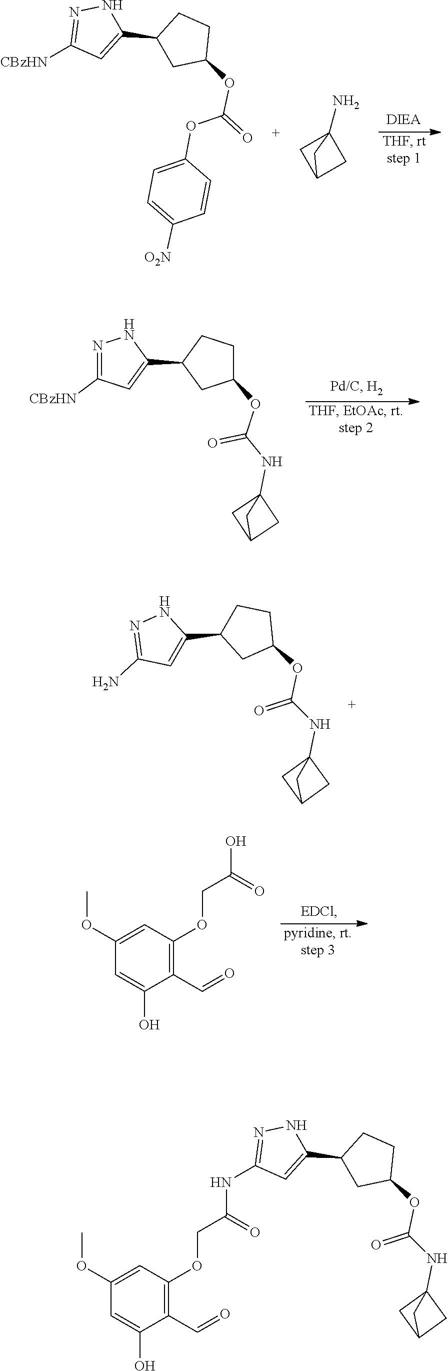

### Step 1: Synthesis of benzyl ({5-[(1S,3R)-3-[({bicyclo[1.1.1]pentan-1-yl}carbamoyl)oxy]cyclopentyl]-1H-pyrazol-3-yl}carbamoyl)formate

To a stirred solution of intermediate C (5.0 g, 10.719 mmol, 1 equiv.) in THF (60 mL) was added bicyclo[1.1.1]pentan-1-amine (2.67 g, 32.157 mmol, 3 equiv.) and DIEA (11.20 mL, 64.314 mmol, 6 equiv.) at 25° C. The resulting mixture was stirred at 25° C. for 4 h. The resulting mixture was diluted with EA (150 mL), washed with 0.5 M NaOH (3×100 mL), and then with brine (2×100 mL). The combined organic extracts were concentrated under reduced pressure. The residue was purified by C18 reverse flash chromatography, water in MeCN, 60-80% gradient to afford benzyl ({5-[(1S,3R)-3-[({bicyclo[1.1.1]pentan-1-yl}carbamoyl)oxy]cyclopentyl]-1H-pyrazol-3-yl}carbamoyl)formate (2.9 g, 49.80% yield) as a white solid.

### Step 2: Synthesis of (1R,3S)-3-(5-amino-2H-pyrazol-3-yl)cyclopentyl N-{bicyclo[1.1.1]pentan-1-yl}carbamate

To a stirred solution of benzyl ({5-[(1S,3R)-3-[({bicyclo[1.1.1]pentan-1-yl}carbamoyl)oxy]cyclopentyl]-1H-pyrazol-3-yl}carbamoyl)formate (2.7 g, 6.158 mmol, 1 equiv.) in THF (15 mL) and EA (15 mL) was added 10% Pd/C (1.90 g) at 25° C. The resulting mixture was stirred at 25° C. for 4 h under H₂ atmosphere. The resulting mixture was filtered, the filter cake was washed with EA (3×30 mL). The filtrate was concentrated under reduced pressure. This resulted in (1R,3S)-3-(5-amino-2H-pyrazol-3-yl)cyclopentyl N-{bicyclo[1.1.1]pentan-1-yl}carbamate (1.6 g, 76.89% yield) as a yellow solid. The crude material was used without any further purification.

### Step 3: Synthesis of 1R,3S)-3-{5-[2-(2-formyl-3-hydroxy-5-methoxyphenoxy)acetamido]-2H-pyrazol-3-yl}cyclopentyl N-{bicyclo[1.1.1]pentan-1-yl}carbamate

To a stirred solution of (1R,3S)-3-(5-amino-2H-pyrazol-3-yl)cyclopentyl N-{bicyclo[1.1.1]pentan-1-yl}carbamate (500.0 mg, 1.809 mmol, 1 equiv.) and intermediate A (409.25 mg, 1.809 mmol, 1 equiv.) in pyridine (8 mL) was added EDCI (520.28 mg, 2.713 mmol, 1.5 equiv.) at 25° C. The resulting mixture was stirred at 25° C. for 3 h. The resulting mixture was diluted with water and extracted with EA (3×30 mL). The combined organic extracts were washed with brine (2×30 mL) and dried over anhydrous sodium sulfate. After filtration, the filtrate was concentrated under reduced pressure. The residue was purified by C18 reverse flash chromatography, 40 to 60% MeCN/water with 0.05% TFA modifier to afford (1R,3S)-3-{5-[2-(2-formyl-3-hydroxy-5-methoxyphenoxy)acetamido]-2H-pyrazol-3-yl}cyclopentyl N-{bicyclo[1.1.1]pentan-1-yl}carbamate (114.0 mg, 10.39% yield) as a white solid. MS (ESI) calcd. for C₂₄H₂₈N₄O₇, 484.20 m/z, found [M+H]⁺ 485.20 m/z. ¹H NMR (300 MHz, DMSO-D6) δ 12.33 (br s, 1H), 10.56 (br s, 1H), 10.15 (s, 1H), 7.77 (d, J=3.0 Hz, 1H), 6.31 (s, 1H), 6.14 (d, J=1.8 Hz, 1H), 6.07 (d, J=1.8 Hz, 1H), 4.95-4.98 (m, 1H), 4.81 (s, 2H), 3.81 (s, 3H), 3.02-3.07 (m, 1H), 2.41-2.49 (m, 1H), 2.30-2.35 (m, 1H), 1.94-2.02 (m, 1H), 1.81-1.89 (m, 7H), 1.58-1.76 (m, 3H).

### Example 4.48: Synthesis of Compound 251: (1R,3S)-3-(3-(2-(2-formyl-3-hydroxy-5-methoxyphenoxy)acetamido)-1H-pyrazol-5-yl)cyclopentyl (1-methylcyclopropyl)carbamate

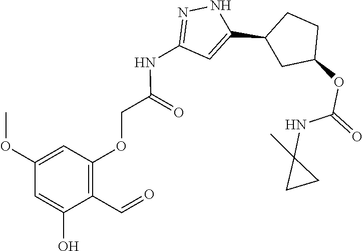 [SMILES: COc1cc(O)c(C=O)c(OCC(=O)Nc2cc([C@H]3CC[C@@H](OC(=O)NC4(C)CC4)C3)[nH]n2)c1]

### Synthetic Route

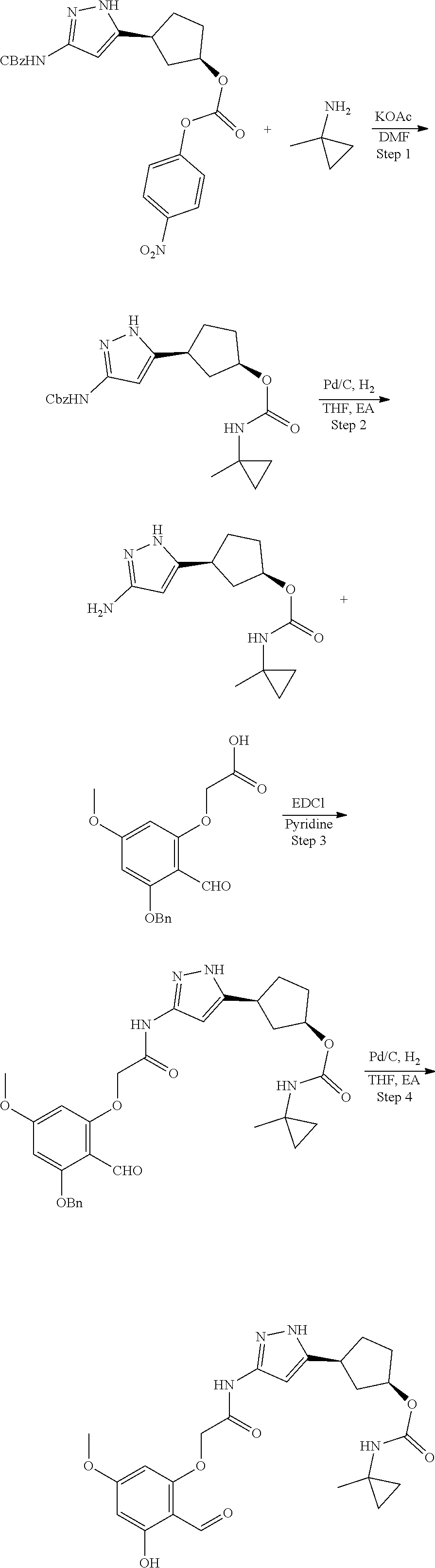

### Step 1: Synthesis of (1R,3S)-3-(3-(((benzyloxy)carbonyl)amino)-1H-pyrazol-5-yl)cyclopentyl (1-methylcyclopropyl)carbamate

To a stirred solution of intermediate C (5 g, 10.719 mmol, 1 equiv.) and 1-methylcyclopropan-1-amine (3.44 g, 32.157 mmol, 3 equiv.) in DMF (80 mL) was added KOAc (2.10 g, 21.438 mmol, 2 equiv.) at room temperature. The resulting mixture was stirred for 1 h at room temperature. The reaction was monitored by LCMS. The reaction was quenched with water (500 mL) at room temperature. The resulting mixture was extracted with EA (3×300 mL). The combined organic extracts were washed with brine (3×300 mL) and dried over anhydrous sodium sulfate. After filtration, the filtrate was concentrated under reduced pressure. The residue was purified by was purified by C18 reverse phase chromatography, 50-70% MeCN/water to afford (1R,3S)-3-(3-(((benzyloxy)carbonyl)amino)-1H-pyrazol-5-yl)cyclopentyl (1-methylcyclopropyl)carbamate (3.9 g, 91% yield) as a yellow solid.

### Step 2: Synthesis of (1R,3S)-3-(3-amino-1H-pyrazol-5-yl)cyclopentyl (1-methylcyclopropyl)carbamate

To a stirred solution of (1R,3S)-3-(3-(((benzyloxy)carbonyl)amino)-1H-pyrazol-5-yl)cyclopentyl (1-methylcyclopropyl)carbamate (3.9 g, 9.788 mmol, 1 equiv.) in THF (80 mL) and EA (30 mL) was added 10% Pd/C (3.0 g) at room temperature. The resulting mixture was stirred for 1 h at room temperature under H₂ atmosphere. The reaction was monitored by LCMS. The resulting mixture was filtered, the filter cake was washed with THF (3×200 mL). The filtrate was concentrated under reduced pressure to afford (1R,3S)-3-(3-amino-1H-pyrazol-5-yl)cyclopentyl (1-methylcyclopropyl)carbamate (2.5 g, 97% yield) as a yellow solid. The crude material was used without any further purification.

### Step 3: Synthesis of (1R,3S)-3-(3-(2-(3-(benzyloxy)-2-formyl-5-methoxyphenoxy)acetamido)-1H-pyrazol-5-yl)cyclopentyl (1-methylcyclopropyl)carbamate

To a stirred solution of (1R,3S)-3-(3-amino-1H-pyrazol-5-yl)cyclopentyl (1-methylcyclopropyl)carbamate (2.5 g, 9.464 mmol, 1 equiv.) and intermediate B (2.99 g, 9.464 mmol, 1.00 equiv.) in pyridine (100 mL) was added EDCI (2.72 g, 14.196 mmol, 1.5 equiv.) at room temperature. The resulting mixture was stirred for 2 h at room temperature. The reaction was monitored by LCMS. The reaction was quenched with water (300 mL) and extracted with EA (3×300 mL). The combined organic extracts were washed with brine (3×300 mL) and dried over anhydrous sodium sulfate. After filtration, the filtrate was concentrated under reduced pressure. The residue by was purified by silica gel column chromatography, 0 to 15% MeOH/DCM to provide (1R,3S)-3-(3-(2-(3-(benzyloxy)-2-formyl-5-methoxyphenoxy)acetamido)-1H-pyrazol-5-yl)cyclopentyl (1-methylcyclopropyl)carbamate (3.0 g, 56% yield) as a yellow solid.

### Step 4: Synthesis of (1R,3S)-3-(3-(2-(2-formyl-3-hydroxy-5-methoxyphenoxy)acetamido)-1H-pyrazol-5-yl)cyclopentyl (1-methylcyclopropyl)carbamate

To a stirred solution of (1R,3S)-3-(3-(2-(3-(benzyloxy)-2-formyl-5-methoxyphenoxy)acetamido)-1H-pyrazol-5-yl)cyclopentyl (1-methylcyclopropyl)carbamate (3 g, 5.332 mmol, 1 equiv.) in THF (60 mL) and EA (10 mL) was added 10% Pd/C (2.25 g) at room temperature. The resulting mixture was stirred for 1 h at room temperature under H₂ atmosphere. The reaction was monitored by LCMS. The resulting mixture was filtered, the filter cake was washed with THF (3×30 mL). The filtrate was concentrated under reduced pressure. The residue by C18 reverse phase chromatography, 45 to 50% MeCN/water with 0.05% TFA modifier to afford (1R,3S)-3-(3-(2-(2-formyl-3-hydroxy-5-methoxyphenoxy)acetamido)-1H-pyrazol-5-yl)cyclopentyl (1-methylcyclopropyl)carbamate (286.4 mg, 9% yield) as an off-white solid. ¹H NMR (400 MHz, DMSO-D6) δ (ppm): 12.33 (s, 1H), 10.56 (s, 1H), 10.15 (s, 1H), 7.36 (s, 1H), 6.30 (s, 1H), 6.14-6.15 (m, 1H), 6.06-6.07 (m, 1H), 4.97-4.98 (m, 1H), 4.81 (s, 2H), 3.81 (s, 3H), 3.02-3.06 (m, 1H), 2.43-2.48 (m, 1H), 1.99-2.08 (m, 1H), 1.83-1.92 (m, 1H), 1.55-1.70 (m, 3H), 1.23 (s, 3H), 0.58 (s, 2H), 0.45-0.47 (m, 2H). MS (ESI) calcd. for C₂₃H₂₈N₄O₇ 472.20 m/z, found [M+H]⁺ 473.10 m/z.

### Example 4.48: Synthesis of Compound 255: (1R,3S)-3-(3-(2-(2-formyl-3-hydroxy-5-methoxyphenoxy)acetamido)-1H-pyrazol-5-yl)cyclopentyl pyrrolidine-1-carboxylate

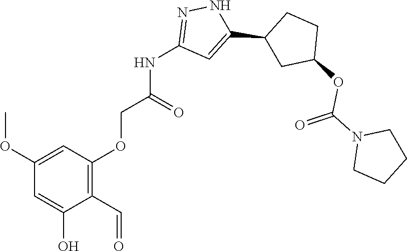 [SMILES: COc1cc(O)c(C=O)c(OCC(=O)Nc2cc([C@H]3CC[C@@H](OC(=O)N4CCCC4)C3)[nH]n2)c1]

### Synthetic Route

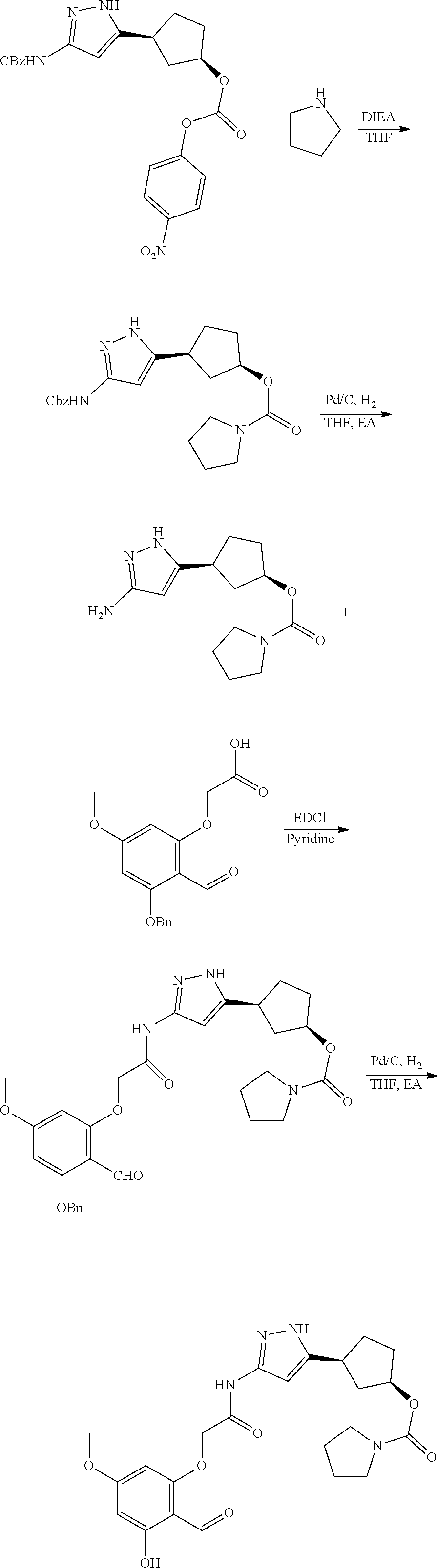

### Step 1: Synthesis of (1R,3S)-3-(3-(((benzyloxy)carbonyl)amino)-1H-pyrazol-5-yl)cyclopentyl pyrrolidine-1-carboxylate

To a stirred solution of intermediate C (60 g, 128.631 mmol, 1 equiv.) in THF (600 mL) was added pyrrolidine (18.30 mL, 257.262 mmol, 2 equiv.) and DIEA (67.22 mL, 385.893 mmol, 3 equiv.) at room temperature. The resulting mixture was stirred for 2 h at room temperature. The reaction was monitored by LCMS. The reaction was quenched by a solution of 0.5 M NaOH aq. (1000 mL) at room temperature. The resulting mixture was extracted with EA (3×1000 mL). The combined organic extracts were washed with brine (2×1000 mL), dried over anhydrous sodium sulfate. After filtration, the filtrate was concentrated under reduced pressure. This resulted in (1R,3S)-3-(3-(((benzyloxy)carbonyl)amino)-1H-pyrazol-5-yl)cyclopentyl pyrrolidine-1-carboxylate (37 g, 72% yield) as yellow oil. The crude material was used without any further purification.

### Step 2: Synthesis of (1R,3S)-3-(3-amino-1H-pyrazol-5-yl)cyclopentyl pyrrolidine-1-carboxylate

To a stirred solution of (1R,3S)-3-(3-(((benzyloxy)carbonyl)amino)-1H-pyrazol-5-yl)cyclopentyl pyrrolidine-1-carboxylate (37 g, 92.857 mmol, 1 equiv.) in THF (150 mL) and EA (300 mL) was added 10% Pd/C (20 g) at room temperature. The resulting mixture was stirred for 4 h under H₂ atmosphere. The reaction was monitored by LCMS. The resulting mixture was filtered, the filter cake was washed with EA (4×150 mL). The filtrate was concentrated under reduced pressure. This resulted in (1R,3S)-3-(3-amino-TH-pyrazol-5-yl)cyclopentyl pyrrolidine-1-carboxylate (23.8 g, 97% yield) as yellow oil. The crude material was used without any further purification.

### Step 3: Synthesis of (1R,3S)-3-(3-(2-(3-(benzyloxy)-2-formyl-5-methoxyphenoxy)acetamido)-1H-pyrazol-5-yl)cyclopentyl pyrrolidine-1-carboxylate

To a stirred solution of intermediate B (1.5 g, 4.742 mmol, 1 equiv.) and (1R,3S)-3-(3-amino-1H-pyrazol-5-yl)cyclopentyl pyrrolidine-1-carboxylate (1.25 g, 4.742 mmol, 1 equiv.) in pyridine (30 mL) was added EDCI (1.82 g, 9.484 mmol, 2 equiv.) at room temperature. The resulting mixture was stirred for 1 h at room temperature. The reaction was monitored by LCMS. The reaction was quenched by water (100 mL) and extracted with EA (3×100 mL). The combined organic extracts were washed with brine (3×100 mL) and dried over anhydrous sodium sulfate. After filtration, the filtrate was concentrated under reduced pressure. The residue was purified by C18 reverse phase chromatography, 10 to 70% MeCN/water with 0.05% TFA modifier to afford (1R,3S)-3-(3-(2-(3-(benzyloxy)-2-formyl-5-methoxyphenoxy)acetamido)-1H-pyrazol-5-yl)cyclopentyl pyrrolidine-1-carboxylate (2 g, 75% yield) as a yellow solid.

### Step 4: Synthesis of (1R,3S)-3-(3-(2-(2-formyl-3-hydroxy-5-methoxyphenoxy)acetamido)-1H-pyrazol-5-yl)cyclopentyl pyrrolidine-1-carboxylate

To a stirred solution of (1R,3S)-3-(3-(2-(3-(benzyloxy)-2-formyl-5-methoxyphenoxy)acetamido)-1H-pyrazol-5-yl)cyclopentyl pyrrolidine-1-carboxylate (300 mg, 0.533 mmol, 1 equiv.) in THF (5 mL) and EA (5 mL) was added 10% Pd/C (150 mg) at room temperature. The resulting mixture was stirred for 1 h at room temperature under H₂ atmosphere. The reaction was monitored by LCMS. The resulting mixture was filtered, the filter cake was washed with EA (3×30 mL). The filtrate was concentrated under reduced pressure. The residue was purified by C18 reverse phase chromatography, 10 to 60% MeCN/water with 0.05% TFA modifier to afford (1R,3S)-3-(3-(2-(2-formyl-3-hydroxy-5-methoxyphenoxy)acetamido)-1H-pyrazol-5-yl)cyclopentyl pyrrolidine-1-carboxylate (135.1 mg, 43% yield) as a white solid. ¹H NMR (500 MHz, DMSO-D6) δ (ppm): 12.32 (s, 1H), 10.54 (s, 1H), 10.15 (s, 1H), 6.32 (s, 1H), 6.14 (s, 1H), 6.07 (s, 1H), 4.99-5.03 (m, 1H), 4.81 (s, 2H), 3.81 (s, 3H), 3.19-3.21 (m, 4H), 3.09-3.12 (m, 1H), 2.36-2.43 (m, 1H), 1.99-2.04 (m, 1H), 1.85-1.90 (m, 1H), 1.66-1.78 (m, 7H). MS (ESI) calcd. for C₂₃H₂₈N₄O₇ 472.20 m/z, found [M+H]⁺ 473.20 m/z.

### Example 5 Representative Schemes Following General Scheme D

### Example 5.1: Synthesis of Compound 17: (1R,3S)-3-{5-[5-(2-formyl-3-hydroxyphenyl)-2-methyl pyrazole-3-amido]-2H-pyrazol-3-yl}cyclopentyl N-isopropylcarbamate 2,2,2-trifluoroacetate

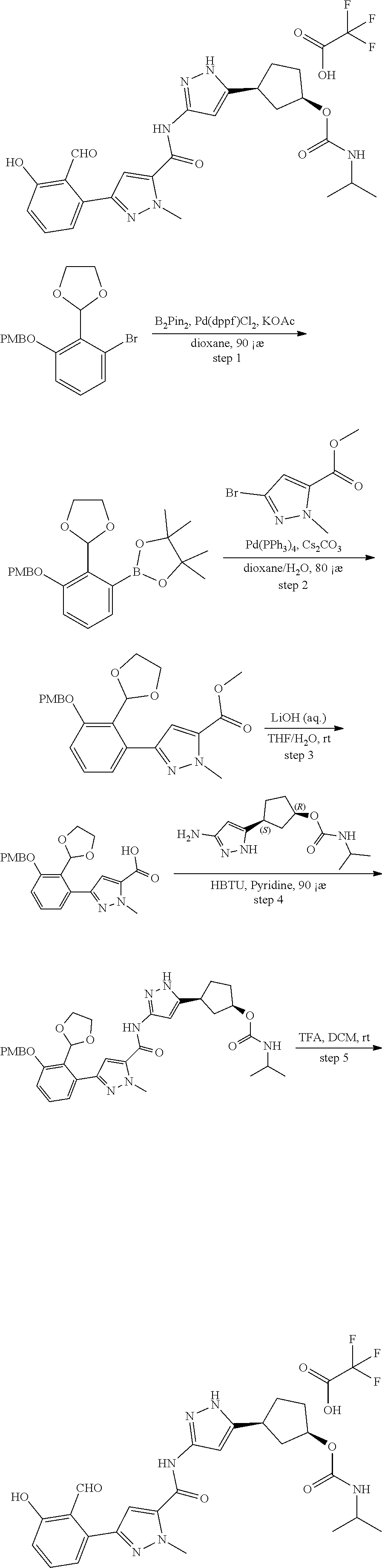

### Step 1: Synthesis of 2-[2-(1,3-dioxolan-2-yl)-3-[(4-methoxyphenyl)methoxy]phenyl]-4,4,5,5-tetramethyl-1,3,2-dioxaborolane

To a stirred solution of 2-{2-bromo-6-[(4-methoxyphenyl)methoxy]phenyl}-1,3-dioxolane (2.0 g, 5.476 mmol, 1 equiv) in dioxane (30 mL) was added 4,4,5,5-tetramethyl-2-(4,4,5,5-tetramethyl-1,3,2-dioxaborolan-2-yl)-1,3,2-dioxaborolane (5.6 g, 21.904 mmol, 4 equiv), Pd(dppf)Cl₂ (0.6 g, 0.821 mmol, 0.15 equiv) and KOAc (1.3 g, 13.690 mmol, 2.5 equiv) at room temperature under N₂ atmosphere. The resulting mixture was stirred for 2 h at 90° C. under N₂ atmosphere, then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by silica gel column chromatography, eluted with EA:PE (1:5) to afford 2-[2-(1,3-dioxolan-2-yl)-3-[(4-methoxy phenyl)methoxy]phenyl]-4,4,5,5-tetramethyl-1,3,2-dioxaborolane (2.0 g, 88.58%) as a light green solid. MS (ESI) calcd. for C₂₃H₂₉BO₆, 412.21 m/z, found 413.20 [M+H]⁺.

### Step 2: Synthesis of methyl 5-[2-(1,3-dioxolan-2-yl)-3-[(4-methoxyphenyl)methoxy]phenyl]-2-methylpyrazole-3-carboxylate

To a stirred solution of methyl 5-bromo-2-methylpyrazole-3-carboxylate (500.0 mg, 2.283 mmol, 1 equiv) and 2-[2-(1,3-dioxolan-2-yl)-3-[(4-methoxyphenyl)methoxy]phenyl]-4,4,5,5-tetramethyl-1,3,2-dioxaborolane (1411.7 mg, 3.425 mmol, 1.5 equiv) in dioxane (10 mL) and H₂O (2 mL) were added Pd(PPh₃)₄ (395.7 mg, 0.342 mmol, 0.15 equiv) and Cs₂CO₃ (1859.4 mg, 5.707 mmol, 2.5 equiv) at room temperature under N₂ atmosphere. The resulting mixture was stirred for 2 h at 80° C., then cooled to room temperature and diluted with water. The resulting mixture was extracted with EA (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with EA:PE (1:5) to afford methyl 5-[2-(1,3-dioxolan-2-yl)-3-[(4-methoxyphenyl)methoxy]phenyl]-2-methyl pyrazole-3-carboxylate (700.0 mg, 72.25%) as a yellow solid. MS (ESI) calcd. for C₂₃H₂₄N₂O₆, 424.16 m/z, found 425.15 [M+H]⁺.

### Step 3: Synthesis of 5-{2-formyl-3-[(4-methoxyphenyl)methoxy]phenyl}-2-methylpyrazole-3-carboxylic acid

To a stirred solution of methyl 5-[2-(1,3-dioxolan-2-yl)-3-[(4-methoxyphenyl)methoxy]phenyl]-2-methylpyrazole-3-carboxylate (660.0 mg, 1.555 mmol, 1 equiv) in THF (2 mL, 0.028 mmol) was added lithium hydroxide (111.7 mg, 4.665 mmol, 3 equiv) in H₂O (2 mL) at room temperature. The resulting mixture was stirred for 1 h. The mixture was acidified to pH 6 with HCl (0.5 M). The resulting mixture was extracted with EtOAc (3×50 mL). The combined organic layers were washed with brine (2×20 mL), dried over anhydrous Na₂SO₄. After filtration, the filtrate was concentrated under reduced pressure. This resulted in 5-{2-formyl-3-[(4-methoxyphenyl)methoxy]phenyl}-2-methylpyrazole-3-carboxylic acid (390.0 mg, 68.46%) as a yellow solid. MS (ESI) calcd. for C₂₂H₂₂N₂O₆, 410.15 m/z, found 411.15 [M+H]⁺.

### Step 4: Synthesis of (1R,3S)-3-(5-{5-[2-(1,3-dioxolan-2-yl)-3-[(4-methoxyphenyl)methoxy]phenyl]-2-methylpyrazole-3-amido}-2H-pyrazol-3-yl)cyclopentyl N-isopropylcarbamate

To a stirred solution of 5-[2-(1,3-dioxolan-2-yl)-3-[(4-methoxyphenyl)methoxy]phenyl]-2-methylpyrazole-3-carboxylic acid (150.0 mg, 0.365 mmol, 1 equiv) and (1R,3S)-3-(5-amino-2H-pyrazol-3-yl)cyclopentyl N-isopropylcarbamate (92.2 mg, 0.365 mmol, 1 equiv) in pyridine (4 mL) were added HBTU (166.3 mg, 0.438 mmol, 1.2 equiv) at room temperature. The resulting mixture was stirred for 2 h at 110° C., then cooled to room temperature and diluted with water. The resulting mixture was extracted with EA (3×20 mL). The combined organic layers were washed with brine (3×20 mL), dried over anhydrous Na₂SO₄. After filtration, the filtrate was concentrated under reduced pressure. The mixture was purified by reversed-phase flash chromatography with the following conditions: column, C18 silica gel; mobile phase, ACN in H₂O, 10% to 50% gradient in 10 min; detector, UV 254 nm. This resulted in (1R,3S)-3-(5-{5-[2-(1,3-dioxolan-2-yl)-3-[(4-methoxyphenyl)methoxy]phenyl]-2-methylpyrazole-3-amido}-2H-pyrazol-3-yl)cyclopentyl N-isopropylcarbamate (40.0 mg, 16.98%) as a light yellow solid. MS (ESI) calcd. for C₃₄H₄₀N₆O₇, 644.30 m/z, found 645.35 [M+H]⁺.

### Step 5: Synthesis of (1R,3S)-3-{5-[5-(2-formyl-3-hydroxyphenyl)-2-methylpyrazole-3-amido]-2H-pyrazol-3-yl}cyclopentyl N-isopropylcarbamate

To a stirred solution of (1R,3S)-3-(5-{5-[2-(1,3-dioxolan-2-yl)-3-[(4-methoxyphenyl)methoxy]phenyl]-2-methylpyrazole-3-amido}-2H-pyrazol-3-yl)cyclopentyl N-isopropylcarbamate (30.0 mg, 0.047 mmol, 1 equiv) in DCM (2 mL, 31.461 mmol) was added TFA (0.2 mL, 2.693 mmol) at room temperature. The resulting mixture was stirred for 1 h. The resulting mixture was concentrated under reduced pressure. The crude product was purified by Prep-HPLC with the following conditions (Column: Xselect CSH C18 OBD Column, 30×150 mm, 5 μm; Mobile Phase A: Water (0.05% TFA), Mobile Phase B: ACN; Flow rate: 60 mL/min; Gradient: 33% B to 63% B in 7 min; Wave Length: 254 nm/220 nm; RT1 (min): 6.05) to afford (1R,3S)-3-{5-[5-(2-formyl-3-hydroxyphenyl)-2-methylpyrazole-3-amido]-2H-pyrazol-3-yl}cyclopentyl N-isopropylcarbamate (6.0 mg, 26.73%) as a white solid. MS (ESI) calcd. for C₂₄H₂₈N₆O₅, 480.21 m/z, found 481.20 [M+H]⁺. ¹H NMR (400 MHz, DMSO-d₆) δ 11.85 (br s, 1H), 10.94 (s, 1H), 10.38 (s, 1H), 7.64-7.68 (m, 1H), 7.49 (s, 1H), 7.16-7.18 (m, 1H), 6.97-7.02 (m, 1H), 6.94-6.95 (m, 1H), 6.46 (m, 1H), 5.00-5.02 (m, 1H), 4.17 (s, 3H), 3.56-3.61 (m, 1H), 3.07-3.11 (m, 1H), 2.51 (m, 1H), 2.02-2.07 (m, 1H), 1.87-1.93 (m, 1H), 1.74-1.76 (m, 2H), 1.63-1.66 (m, 1H), 1.04 (d, J=6.4 Hz, 6H). ¹⁹F NMR (376 MHz, DMSO-d₆) δ −74.57.

### Example 5.2: Synthesis of Compound 18: (1R,3S)-3-{5-[5-(3-formyl-2-hydroxyphenyl)-2-methyl pyrazole-3-amido]-2H-pyrazol-3-yl}cyclopentyl N-isopropylcarbamate trifluoroacetic acid

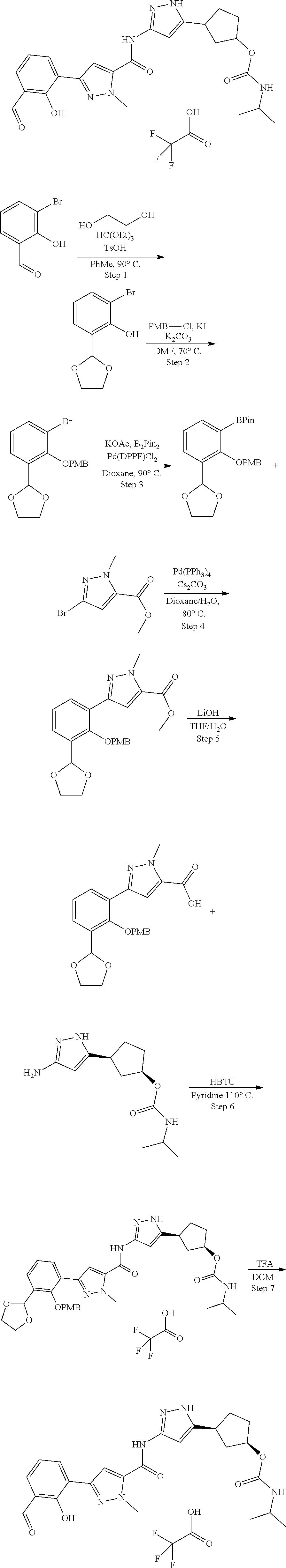

### Step 1: Synthesis of 2-bromo-6-(1,3-dioxolan-2-yl)phenol

To a stirred solution of 3-bromo-2-hydroxybenzaldehyde (12 g, 59.696 mmol, 1 equiv) in toluene (160 mL, 1503.782 mmol, 25.19 equiv) was added ethylene glycol (18.53 g, 298.480 mmol, 5 equiv), triethyl orthoformate (26.54 g, 179.088 mmol, 3 equiv) and para-toluene sulfonic acid (1.03 g, 5.970 mmol, 0.1 equiv) at room temperature. The resulting mixture was stirred for 12 h at 90° C., then cooled to room temperature and diluted with water. The resulting mixture was extracted with EA (3×200 mL). The combined organic layers were washed with brine (3×200 mL), dried over anhydrous Na₂SO₄. After filtration, the filtrate was concentrated under reduced pressure. The residue was purified by silica gel column chromatography, eluted with PE and EA (5:1) to afford 2-bromo-6-(1,3-dioxolan-2-yl)phenol (9.7 g, 66.30%) as a white solid. MS (ESI) calcd. for C₉H₉BrO₃, 243.97 m/z, found 245.05 [M+H]⁺.

### Step 2: Synthesis of 2-(3-bromo-2-((4-methoxybenzyl)oxy)phenyl)-1,3-dioxolane

To a stirred solution of 2-bromo-6-(1,3-dioxolan-2-yl)phenol (23 g, 93.850 mmol, 1 equiv) in DMF (200 mL) was added K₂CO₃ (45.40 g, 328.475 mmol, 3.5 equiv), KI (3.12 g, 18.770 mmol, 0.2 equiv) and PMBCl (17.64 g, 112.620 mmol, 1.2 equiv) at room temperature. The resulting mixture was stirred overnight at 70° C., then cooled to room temperature and diluted with water. The resulting mixture was extracted with EA (3×200 mL). The combined organic layers were washed with brine (3×200 mL), dried over anhydrous Na₂SO₄. After filtration, the filtrate was concentrated under reduced pressure. The crude product was purified by silica gel column chromatography, eluted with EA:PE (1:5) to afford 2-{3-bromo-2-[(4-methoxyphenyl)methoxy]phenyl}-1,3-dioxolane (32.8 g, 92.86%) as light yellow oil. MS (ESI) calcd. for C₁₇H₁₇BrO₄, 364.03 m/z, found 363.05 [M−H]⁻.

### Step 3: Synthesis of 2-(3-(1,3-dioxolan-2-yl)-2-((4-methoxybenzyl)oxy)phenyl)-4,4,5,5-tetramethyl-1,3,2-dioxaborolane

To a stirred solution of 2-{3-bromo-2-[(4-methoxyphenyl)methoxy]phenyl}-1,3-dioxolane (2 g, 5.476 mmol, 1 equiv) in anhydrous dioxane (50 mL) was added bis(pinacolato)diboron (5.56 g, 21.904 mmol, 4 equiv) and KOAc (1.34 g, 13.690 mmol, 2.5 equiv) followed by catalytic amount of Pd(dppf)Cl₂ (0.60 g, 0.821 mmol, 0.15 equiv) at room temperature. The reaction mixture was stirred at 90° C. for a period of 2 h under N₂ atmosphere, then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (1:2) to afford 2-[3-(1,3-dioxolan-2-yl)-2-[(4-methoxyphenyl)methoxy]phenyl]-4,4,5,5-tetramethyl-1,3,2-dioxaborolane (1.2 g, 53.15%) as white oil. MS (ESI) calcd. for C₂₃H₂₉BO₆, 412.21 m/z, found 413.15 [M+H]⁺.

### Step 4: Synthesis of methyl 5-[3-(1,3-dioxolan-2-yl)-2-[(4-methoxyphenyl)methoxy]phenyl]-2-methylpyrazole-3-carboxylate

To a stirred solution of 2-[3-(1,3-dioxolan-2-yl)-2-[(4-methoxyphenyl)methoxy]phenyl]-4,4,5,5-tetramethyl-1,3,2-dioxaborolane (1.2 g, 2.911 mmol, 1.2 equiv) and methyl 5-bromo-2-methylpyrazole-3-carboxylate (0.5 g, 2.426 mmol, 1 equiv) in dioxane (10 mL) and H₂O (2 mL) was added Pd(PPh₃)₄ (0.4 g, 0.364 mmol, 0.15 equiv), and Cs₂CO₃ (1.9 g, 6.065 mmol, 2.5 equiv) at room temperature. The resulting mixture was stirred for 2 h at 80° C. under N₂ atmosphere, then cooled to room temperature and diluted with water. The resulting mixture was extracted with EA (3×200 mL). The combined organic layers were washed with brine (3×200 mL), dried over anhydrous Na₂SO₄. After filtration, the filtrate was concentrated under reduced pressure. The mixture was purified by silica gel column chromatography, eluted with EA:PE (1:4) to methyl 5-[3-(1,3-dioxolan-2-yl)-2-[(4-methoxyphenyl)methoxy]phenyl]-2-methylpyrazole-3-carboxylate (0.7 g, 67.99%) as a light yellow solid. MS (ESI) calcd. for C₂₃H₂₄N₂O₆, 424.16 m/z, found 425.15 [M+H]⁺.

### Step 5: Synthesis of 5-[3-(1,3-dioxolan-2-yl)-2-[(4-methoxyphenyl)methoxy]phenyl]-2-methylpyrazole-3-carboxylic acid

To a stirred solution of methyl 5-[3-(1,3-dioxolan-2-yl)-2-[(4-methoxyphenyl)methoxy]phenyl]-2-methylpyrazole-3-carboxylate (650.0 mg, 1.531 mmol, 1 equiv) in H₂O (3 mL) was added LiOH (91.7 mg, 3.827 mmol, 2.5 equiv) in THF (3 mL) at room temperature. The resulting mixture was stirred for 2 h. The mixture was acidified to pH 6 with HCl (1 moL/L). The aqueous layer was extracted with EA (3×50 mL). The resulting mixture was concentrated under reduced pressure to afford 5-[3-(1,3-dioxolan-2-yl)-2-[(4-methoxyphenyl)methoxy]phenyl]-2-methylpyrazole-3-carboxylic acid (400.0 mg, 63.64%) as a light yellow solid. MS (ESI) calcd. for C₂₂H₂₂N₂O₆, 410.15 m/z, found 411.15 [M+H]⁺.

### Step 6: Synthesis of (1R,3S)-3-(5-{5-[3-(1,3-dioxolan-2-yl)-2-[(4-methoxyphenyl)methoxy]phenyl]-2-methyl pyrazole-3-amido}-2H-pyrazol-3-yl)cyclopentyl N-isopropylcarbamate

To a stirred solution of 5-[3-(1,3-dioxolan-2-yl)-2-[(4-methoxyphenyl)methoxy]phenyl]-2-methylpyrazole-3-carboxylic acid (300.0 mg, 0.731 mmol, 1.3 equiv) and (1R,3S)-3-(5-amino-2H-pyrazol-3-yl)cyclopentyl N-isopropylcarbamate (141.8 mg, 0.562 mmol, 1 equiv) in pyridine (5 mL) was added HBTU (277.2 mg, 0.731 mmol, 1.3 equiv) at room temperature. The resulting mixture was stirred overnight at 110° C., then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by silica gel column chromatography, eluted with EA:PE (3:2) to afford (1R,3S)-3-(5-{5-[3-(1,3-dioxolan-2-yl)-2-[(4-methoxyphenyl)methoxy]phenyl]-2-methylpyrazole-3-amido}-2H-pyrazol-3-yl)cyclopentyl N-isopropylcarbamate (150.0 mg, 41.38%) as a light yellow solid. MS (ESI) calcd. for C₃₄H₄₀N₆O₇, 644.10 m/z, found 645.35 [M+H]⁺.

### Step 7: Synthesis of (1R,3S)-3-{5-[5-(3-formyl-2-hydroxyphenyl)-2-methylpyrazole-3-amido]-2H-pyrazol-3-yl}cyclopentyl N-isopropylcarbamate trifluoroacetic acid

To a stirred solution (1R,3S)-3-(5-{5-[3-(1,3-dioxolan-2-yl)-2-[(4-methoxyphenyl)methoxy]phenyl]-2-methylpyrazole-3-amido}-2H-pyrazol-3-yl)cyclopentyl N-isopropylcarbamate (50.0 mg, 0.078 mmol, 1 equiv) in DCM (3.00 mL, 47.465 mmol, 608.52 equiv) were added TFA (0.50 mL, 6.770 mmol, 86.80 equiv) dropwise at room temperature. The resulting mixture was stirred for 0.5 h. The resulting mixture was concentrated under reduced pressure. The crude product was purified by Prep-HPLC with the following conditions (Column: XSelect CSH Fluoro Phenyl 5 m, 19 mm×250 mm; Mobile Phase A: Water (0.05% TFA), Mobile Phase B: ACN; Flow rate: 25 mL/min; Gradient: 38% B to 62% B in 9.5 min; Wave Length: 254 nm/220 nm; RT1 (min): 8.35) to afford (1R,3S)-3-{5-[5-(3-formyl-2-hydroxyphenyl)-2-methylpyrazole-3-amido]-2H-pyrazol-3-yl}cyclopentyl N-isopropylcarbamate trifluoroacetic acid (11.2 mg, 24.29%) as a white solid. MS (ESI) calcd. for C₂₄H₂₈N₆O₅, 480.21 m/z, found 481.20 [M+H]⁺. ¹H NMR (400 MHz, DMSO-d₆) δ 12.91 (br s, 1H), 10.21 (s, 1H), 8.30 (br s, 1H), 8.21 (d, J=5.2 Hz, 1H), 7.81 (d, J=5.2 Hz, 1H), 7.72 (s, 1H), 7.56-7.61 (m, 2H), 7.48-7.51 (m, 1H), 7.18 (d, J=8.0 Hz, 1H), 6.90-6.93 (m, 1H), 4.94 (br s, 1H), 4.20 (s, 2H), 3.51 (s, 2H), 2.21-2.50 (m, 8H), 2.16 (s, 3H), 1.23 (s, 6H). ¹⁹F NMR (376 MHz, DMSO-d₆) δ −74.84.

### Example 5.3: Synthesis of Compound 53: (1R,3S)-3-(3-(2-(2-formyl-3-hydroxyphenyl)-4-methylthiazole-5-carboxamido)-1H-pyrazol-5-yl)cyclopentyl isopropylcarbamate

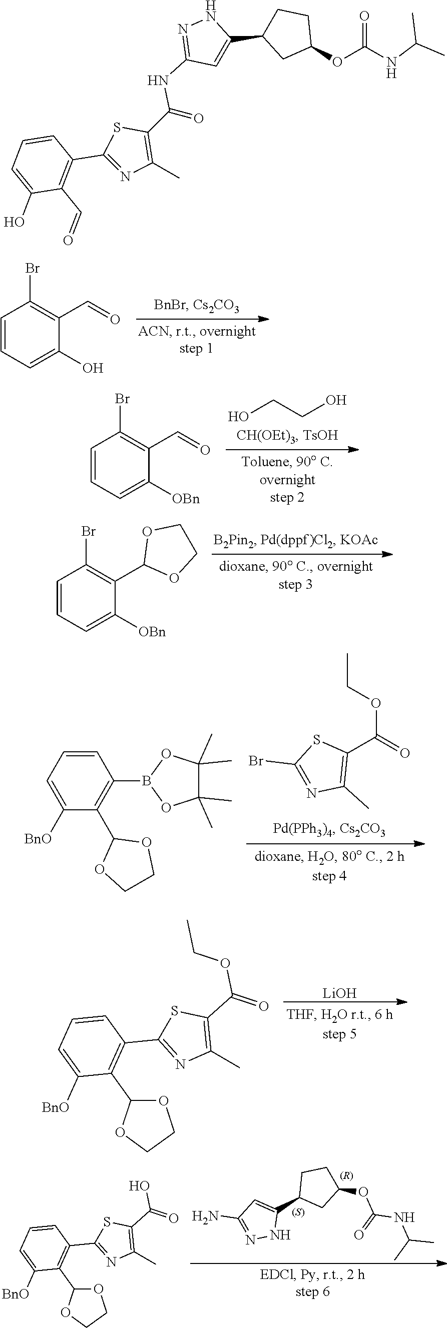

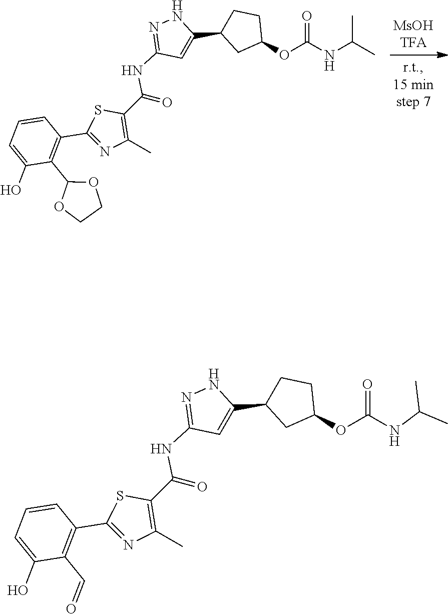 [SMILES: Cc1nc(-c2cccc(O)c2C2OCCO2)sc1C(=O)Nc1cc([C@H]2CC[C@@H](OC(=O)NC(C)C)C2)[nH]n1 ; Cc1nc(-c2cccc(O)c2C=O)sc1C(=O)Nc1cc([C@H]2CC[C@@H](OC(=O)NC(C)C)C2)[nH]n1]

### Step 1: Synthesis of 2-(benzyloxy)-6-bromobenzaldehyde

To a 500 mL flask were added 2-bromo-6-hydroxybenzaldehyde (10.0 g, 49.747 mmol), Cs₂CO₃ (32.4 g, 99.494 mmol), a stir bar and MeCN (150 mL) and stirred until homogenous, then treated with benzyl bromide (12.8 g, 74.620 mmol). The resulting mixture was stirred at rt. overnight. The reaction was quenched with NH₄Cl solution at 0° C. and extracted with EA. The organic layers were combined, dried over Na₂SO₄, filtered and concentrated. The residue obtained was purified by silica gel chromatography (PE/EA=0-15%) to afford 2-(benzyloxy)-6-bromobenzaldehyde (12.8 g, 88.38%) as a white solid. MS (ESI) calcd. for C₁₄H₁₁BrO₂, 289.99 m/z, found: 290.90[M+H]⁺.

### Step 2: Synthesis of 2-(2-(benzyloxy)-6-bromophenyl)-1,3-dioxolane

2-(Benzyloxy)-6-bromobenzaldehyde (12.8 g, 43.964 mmol), triethyl orthoformate (19.6 g, 131.892 mmol), ethylene glycol (13.6 g, 219.820 mmol), para-toluene sulfonate (1.5 g, 8.793 mmol), a stir bar and toluene (150 mL) were added to a 500 mL flask. The resulting mixture was stirred at 90° C. overnight. The reaction was concentrated. The residue obtained was purified by silica gel chromatography (PE/EA=0-15%) to afford 2-[2-(benzyloxy)-6-bromophenyl]-1,3-dioxolane (14.0 g, 95.00%) as yellow oil. MS (ESI) calcd. for C₁₆H₁₅BrO₃, 334.02 m/z, found: 334.90 [M+H]⁺.

### Step 3: Synthesis of 2-(3-(benzyloxy)-2-(1,3-dioxolan-2-yl)phenyl)-4,4,5,5-tetramethyl-1,3,2-dioxaborolane

2-[2-(Benzyloxy)-6-bromophenyl]-1,3-dioxolane (13.5 g, 40.275 mmol), bis(pinacolato)diboron (20.5 g, 80.550 mmol), potassium acetate (11.9 g, 121.228 mmol), Pd(dppf)Cl₂ (1.5 g, 2.014 mmol), a stir bar and 1,4-dioxane (150 mL) were added to a 500 mL flask. The resulting mixture was maintained under nitrogen and stirred at 90° C. overnight. The reaction was quenched with H₂O and extracted with EA. The organic layers were combined, dried over Na₂SO₄, filtered and concentrated. The residue obtained was purified by silica gel chromatography (PE/EA=0-25%) to afford 2-[3-(benzyloxy)-2-(1,3-dioxolan-2-yl)phenyl]-4,4,5,5-tetramethyl-1,3,2-dioxaborolane (13.9 g, 90.29%) as a yellow solid. MS (ESI) calcd. for C₂₂H₂₇BO₅, 382.20 m/z, found: 383.15 [M+H]⁺.

### Step 4: Synthesis of ethyl 2-(3-(benzyloxy)-2-(1,3-dioxolan-2-yl)phenyl)-4-methylthiazole-5-carboxylate

Ethyl 2-bromo-4-methyl-1,3-thiazole-5-carboxylate (250.0 mg, 1.000 mmol), 2-[3-(benzyloxy)-2-(1,3-dioxolan-2-yl)phenyl]-4,4,5,5-tetramethyl-1,3,2-dioxaborolane (420.0 mg, 1.099 mmol), Cs₂CO₃ (817.0 mg, 2.500 mmol), tetrakis(triphenylphosphine)palladium(0) (115.0 mg, 0.100 mmol), a stir bar, H₂O (2 mL) and 1,4-dioxane (10 mL) were added to a 40 mL vial. The resulting mixture was maintained under nitrogen and stirred at 80° C. for 2 h. The reaction was quenched with H₂O and extracted with EA. The organic layers were combined, dried over Na₂SO₄, filtered and concentrated. The residue obtained was purified by silica gel chromatography (PE/EA=0-30%) to afford ethyl 2-[3-(benzyloxy)-2-(1,3-dioxolan-2-yl)phenyl]-4-methyl-1,3-thiazole-5-carboxylate (260.0 mg, 61.13%) as yellow oil. MS (ESI) calcd. for C₂₃H₂₃NO₅S, 425.13 m/z, found: 426.05 [M+H]⁺.

### Step 5: Synthesis of 2-(3-(benzyloxy)-2-(1,3-dioxolan-2-yl)phenyl)-4-methylthiazole-5-carboxylic acid

Ethyl 2-[3-(benzyloxy)-2-(1,3-dioxolan-2-yl)phenyl]-4-methyl-1,3-thiazole-5-carboxylate (260.0 mg, 0.611 mmol), EtOH (1 mL), a stir bar and THF (3 mL) were added to a 50 mL flask and stirred until homogenous, then treated with lithium hydroxide (0.6 mL, 1.800 mmol, 3 M). The resulting mixture was stirred at rt. for 6 h. The reaction was concentrated and diluted with H₂O. The pH of the aqueous phase was adjusted to 3-4 with AcOH (10%). The mixture was extracted with MeOH/DCM (1:10). The organic layers were combined, dried over Na₂SO₄, filtered and concentrated to afford 2-[3-(benzyloxy)-2-(1,3-dioxolan-2-yl)phenyl]-4-methyl-1,3-thiazole-5-carboxylic acid (220.0 mg, 90.59%) as a yellow solid. MS (ESI) calcd. for C₂₁H₁₉NO₅S, 397.10, found, 398.00 [M+H]⁺.

### Step 6: Synthesis of (1R,3S)-3-(3-(2-(3-(benzyloxy)-2-(1,3-dioxolan-2-yl)phenyl)-4-methylthiazole-5-carboxamido)-1H-pyrazol-5-yl)cyclopentyl isopropylcarbamate

(1R,3S)-3-(5-amino-2H-pyrazol-3-yl)cyclopentyl N-isopropylcarbamate (90.0 mg, 0.357 mmol), 2-[3-(benzyloxy)-2-(1,3-dioxolan-2-yl)phenyl]-4-methyl-1,3-thiazole-5-carboxylic acid (170.0 mg, 0.428 mmol), a stir bar and pyridine (2 mL) were added to a 100 mL flask and stirred until homogenous, then treated with EDCI (137.1 mg, 0.715 mmol). The resulting mixture was stirred at rt. for 2 h. The reaction was concentrated. The residue obtained was purified by reverse phase chromatography ACN/H₂O (10 mM NH₄HCO₃)=5-60% to afford (1R,3S)-3-(5-{2-[3-(benzyloxy)-2-(1,3-dioxolan-2-yl)phenyl]-4-methyl-1,3-thiazole-5-amido}-2H-pyrazol-3-yl) cyclopentyl N-isopropylcarbamate (90.0 mg, 39.94%) as an off-white solid. MS (ESI) calcd. for C₃₃H₃₇N₅O₆S, 631.25 m/z, found: 632.30 [M+H]⁺.

### Step 7: Synthesis of (1R,3S)-3-(3-(2-(2-formyl-3-hydroxyphenyl)-4-methylthiazole-5-carboxamido)-1H-pyrazol-5-yl)cyclopentyl isopropylcarbamate

(1R,3S)-3-(5-{2-[3-(benzyloxy)-2-(1,3-dioxolan-2-yl)phenyl]-4-methyl-1,3-thiazole-5-amido}-2H-pyrazol-3-yl)cyclopentyl N-isopropylcarbamate (90.0 mg, 0.142 mmol), trifluoroacetaldehyde (2.5 mL), a stir bar and methanesulfonic acid (0.5 mL) were added to a 50 mL flask. The resulting mixture was stirred at rt. for 15 min, then concentrated at 0° C. The residue obtained was purified by Prep-HPLC (Column: Xselect CSH OBD Column, 30×150 mm, 5 um; Mobile Phase A: Water (0.05% TFA), Mobile Phase B: ACN; Flow rate: 60 mL/min; Gradient: 34% B to 58% B in 10 min; Wave Length: 254/220 nm; RT1 (min): 9) to afford (1R,3S)-3-{5-[2-(2-formyl-3-hydroxyphenyl)-4-methyl-1,3-thiazole-5-amido]-2H-pyrazol-3-yl}cyclopentyl N-isopropyl carbamate (40.6 mg, 57.06%) as a light yellow solid. MS (ESI) calcd. for C₂₄H₂₇N₅O₅S, 497.17 m/z, found, 498.20 [M+H]⁺. ¹H NMR (400 MHz, DMSO-d₆) δ 11.66 (s, 1H), 10.81 (s, 1H), 10.41 (s, 1H), 7.69 (t, J=8.0 Hz, 1H), 7.30 (d, J=7.5 Hz, 1H), 7.23-7.10 (m, 1H), 7.05-6.87 (m, 1H), 6.40 (s, 1H), 5.02 (s, 1H), 3.66-3.48 (m, 1H), 3.16-3.00 (m, 1H), 2.65 (s, 3H), 2.50-2.45 (m, 1H), 2.11-1.84 (m, 2H), 1.83-1.57 (m, 3H), 1.04 (d, J=6.6 Hz, 6H).

### Example 5.4: Synthesis of Compound 55: (1R,3S)-3-{5-[2-(2-formyl-3-hydroxyphenyl)-1,3-oxazole-5-amido]-2H-pyrazol-3-yl}cyclopentyl N-isopropylcarbamat

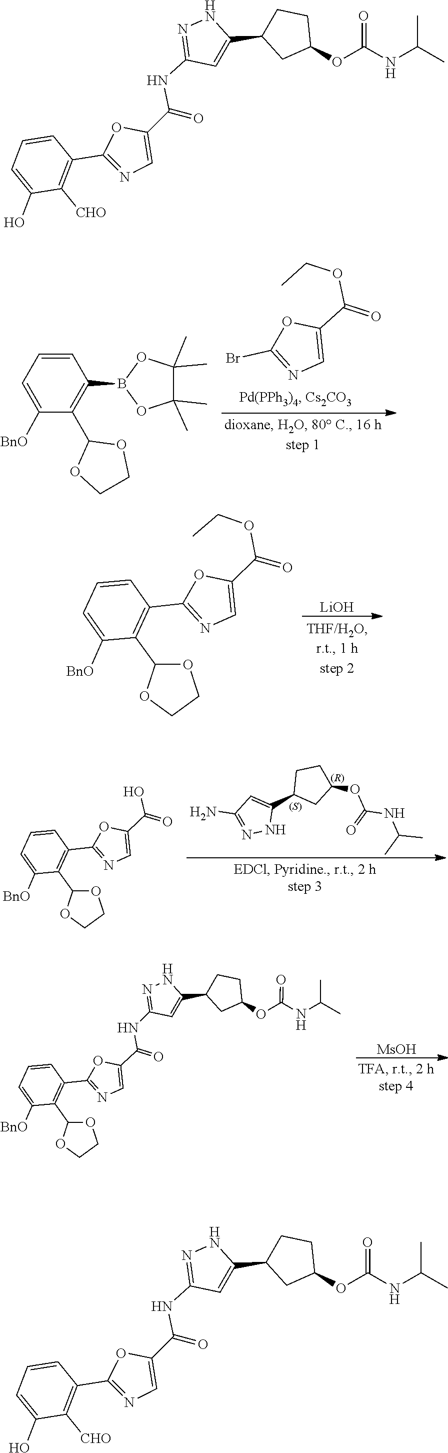

### Step 1: Synthesis of Ethyl 2-[3-(benzyloxy)-2-(1,3-dioxolan-2-yl)phenyl]-1,3-oxazole-5-carboxylate

2-[3-(benzyloxy)-2-(1,3-dioxolan-2-yl)phenyl]-4,4,5,5-tetramethyl-1,3,2-dioxaborolane (1.9 g, 5.000 mmol), a stir bar, ethyl 2-bromo-1,3-oxazole-5-carboxylate (1 g, 4.545 mmol), Cs₂CO₃ (3.7 g, 11.363 mmol) and dioxane (30 mL), water (6 mL) were added to a 100 mL round-bottom flask and stirred until homogenous, then treated with tetrakis(triphenylphosphine)palladium(0) (0.5 g, 0.455 mmol). The resulting mixture was stirred at 80° C. overnight under N₂ atmosphere. The reaction was quenched with H₂O and extracted with EA. The organic layers were combined, dried over Na₂SO₄, filtered and concentrated. The residue obtained was purified by silica gel chromatography (PE/EA=0-60%) to afford ethyl 2-[3-(benzyloxy)-2-(1,3-dioxolan-2-yl)phenyl]-1,3-oxazole-5-carboxylate (263 mg, 15.17%) as a yellow oil. MS (ESI) calcd. for C₂₂H₂₁NO₆, 395.13, found: 396.20 [M+H]⁺.

### Step 2: Synthesis of 2-[3-(benzyloxy)-2-(1,3-dioxolan-2-yl)phenyl]-1,3-oxazole-5-carboxylic acid

Ethyl 2-[3-(benzyloxy)-2-(1,3-dioxolan-2-yl)phenyl]-1,3-oxazole-5-carboxylate (258 mg, 0.652 mmol), lithium hydroxide (21 mg, 0.913 mmol) and tetrahydrofuran (5 mL), water (1 mL) were added to a 100 mL round-bottom flask and stirred until homogeneous. The resulting mixture was stirred at rt. for 1 h. The resulting mixture was concentrated under reduced pressure. The mixture/residue was acidified to pH=5 with HCl (2 M). The resulting mixture was extracted with EA (3×70 mL). The combined organic layers were washed with brine, dried over anhydrous sodium sulfate, filtered and concentrated under vacuum to afford 2-[3-(benzyloxy)-2-(1,3-dioxolan-2-yl)phenyl]-1,3-oxazole-5-carboxylic acid (220 mg, 91.78%) as a yellow solid. MS (ESI) calcd. for C₂₀H₁₇NO₆, 367.10 m/z, found 368.10 [M+H]⁺.

### Step 3: Synthesis of (1R,3S)-3-(5-{2-[3-(benzyloxy)-2-(1,3-dioxolan-2-yl)phenyl]-1,3-oxazole-5-amido}-2H-pyrazol-3-yl)cyclopentyl N-isopropylcarbamate

2-[3-(benzyloxy)-2-(1,3-dioxolan-2-yl)phenyl]-1,3-oxazole-5-carboxylic acid (132 mg, 0.360 mmol), (1R,3S)-3-(5-amino-2H-pyrazol-3-yl)cyclopentyl N-isopropylcarbamate (70 mg, 0.277 mmol) and pyridine (3 mL) were added to a 8 mL vail and stirred until homogeneous, then treated with EDCI (63 mg, 0.332 mmol). The reaction mixture was stirred at rt. for 2 h. The reaction was quenched with H₂O and extracted with EA. The combined organic layers were washed with brine, dried over anhydrous sodium sulfate, filtered, and concentrated under vacuum. The residue was purified by reverse-phase chromatography (5-60%, ACN/5 mM TFA water) to afford (1R,3S)-3-(5-{2-[3-(benzyloxy)-2-(1,3-dioxolan-2-yl)phenyl]-1,3-oxazole-5-amido}-2H-pyrazol-3-yl)cyclopentyl N-isopropylcarbamate (60 mg, 35.95%) as a yellow solid. MS (ESI) mass calcd. for C₃₂H₃₅N₅O₇, 601.25 m/z, found 602.30 [M+H]⁺.

### Step 4: Synthesis of (1R,3S)-3-{5-[2-(2-formyl-3-hydroxyphenyl)-1,3-oxazole-5-amido]-2H-pyrazol-3-yl}cyclopentyl N-isopropylcarbamate

(1R,3S)-3-(5-{2-[3-(benzyloxy)-2-(1,3-dioxolan-2-yl)phenyl]-1,3-oxazole-5-amido}-2H-pyrazol-3-yl)cyclopentyl N-isopropylcarbamate (70 mg, 0.116 mmol), methanesulfonic acid (1.2 mL) and TFA (3.6 mL) were added to a 20 mL vail and stirred until homogeneous. The reaction mixture was stirred at rt. for 2 h and concentrated under vacuum. The residue was purified by Prep-HPLC (Column: Xselect CSH OBD Column, 30×150 mm, 5 um; Mobile Phase A: Water (0.05% TFA), Mobile Phase B: ACN; Flow rate: 60 mL/min; Gradient: 31% B to 55% B in 9 min; Wave Length: 254/220 nm; RT1 (min): 8) to afford (1R,3S)-3-{5-[2-(2-formyl-3-hydroxyphenyl)-1,3-oxazole-5-amido]-2H-pyrazol-3-yl}cyclopentyl N-isopropylcarbamate as a white solid (47.5 mg, 87.33%). MS (ESI) mass calcd. for C₂₃H₂₅N₅O₆, 467.18 m/z, found 468.15 [M+H]⁺. ¹H NMR (400 MHz, DMSO-d₆) δ 11.82-11.69 (m, 1H), 11.21-11.14 (m, 1H), 10.75-10.70 (m, 1H), 8.19 (s, 1H), 7.80-7.69 (m, 2H), 7.26-7.16 (m, 1H), 7.02-6.93 (m, 1H), 6.47-6.40 (m, 1H), 5.07-4.93 (m, 1H), 3.65-3.49 (m, 1H), 3.16-3.03 (m, 1H), 2.50-2.46 (m, 2H), 2.12-2.01 (m, 1H), 1.98-1.86 (m, 1H), 1.82-1.55 (m, 3H), 1.04 (d, J=6.5 Hz, 6H).

### Example 5.5: Synthesis of Compound 60: (1R,3S)-3-{5-[2-ethyl-5-(2-formyl-3-hydroxyphenyl) pyrazole-3-amido]-2H-pyrazol-3-yl}cyclopentyl N-isopropylcarbamate; trifluoroacetic acid

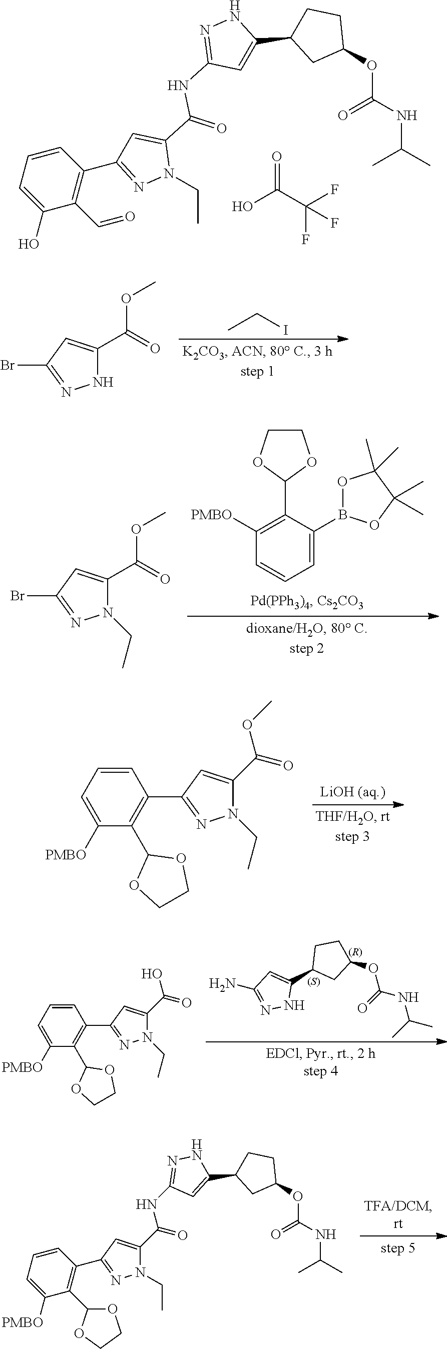

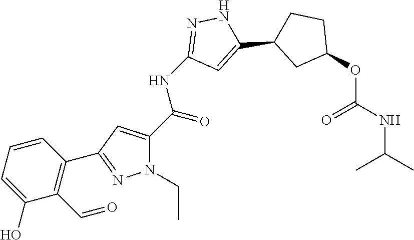 [SMILES: CCn1nc(-c2cccc(O)c2C=O)cc1C(=O)Nc1cc([C@H]2CC[C@@H](OC(=O)NC(C)C)C2)[nH]n1]

### Step 1: Synthesis of methyl 3-bromo-1-ethyl-1H-pyrazole-5-carboxylate

To a stirred solution of methyl 5-bromo-2H-pyrazole-3-carboxylate (2.33 g, 11.365 mmol, 1 equiv) and K₂CO₃ (4.71 g, 34.095 mmol, 3 equiv) in ACN (50 mL) was added ethyl iodide (5.32 g, 34.095 mmol, 3 equiv) dropwise at room temperature. The resulting mixture was stirred for 3 h at 80° C. The reaction mixture was then treated with H₂O (100 mL), dropwise over 10 min, extracted with EtOAc (100 mL×2), and the combined extracts washed with brine (100 mL), dried over anhydrous Na₂SO₄, filtered, and concentrated to dryness in vacuo to give a nearly yellow viscous oil. The oil was then subjected to silica gel chromatography (0-10% EA/petroleum ether) to give methyl 3-bromo-1-ethyl-1H-pyrazole-5-carboxylate (1.42 g, 49.57%) as a colorless oil. MS (ESI) calcd. for C₇H₉BrN₂O₂, 231.98 m/z, found 234.95 [M+H+2]⁺.

### Step 2: Synthesis of methyl 3-(2-(1,3-dioxolan-2-yl)-3-((4-methoxybenzyl)oxy)phenyl)-1-ethyl-1H-pyrazole-5-carboxylate

To a solution of methyl 5-bromo-2-ethylpyrazole-3-carboxylate (700.0 mg, 3.003 mmol, 1 equiv) and 2-[2-(1,3-dioxolan-2-yl)-3-[(4-methoxyphenyl)methoxy]phenyl]-4,4,5,5-tetramethyl-1,3,2-dioxaborolane (1238.3 mg, 3.003 mmol, 1 equiv) in dioxane (20 mL) and H₂O (5 mL) were added Pd(PPh₃)₄ (347.1 mg, 0.300 mmol, 0.1 equiv) and Cs₂CO₃ (2446.5 mg, 7.508 mmol, 2.5 equiv). The reaction was stirred for 2 h at 90° C. under a nitrogen atmosphere. The reaction mixture was then treated with H₂O (20 mL), dropwise over 10 min, extracted with EtOAc (20 mL×2), and the combined extracts washed with brine (20 mL), dried over anhydrous Na₂SO₄ filtered, and concentrated to dryness in vacuo to give a nearly yellow viscous oil. The oil was then subjected to silica gel chromatography (0-30% EtOAc/petroleum ether) to give methyl 3-(2-(1,3-dioxolan-2-yl)-3-((4-methoxybenzyl)oxy)phenyl)-1-ethyl-1H-pyrazole-5-carboxylate (500 mg, 27.68%) as a yellow oil. MS (ESI) calcd. for C₂₄H₂₆N₂O₆, 438.18 m/z, found 439.25 [M+H]⁺.

### Step 3: Synthesis of 3-(2-(1,3-dioxolan-2-yl)-3-((4-methoxybenzyl)oxy)phenyl)-1-ethyl-1H-pyrazole-5-carboxylic acid

To a stirred solution of methyl 5-[2-(1,3-dioxolan-2-yl)-3-[(4-methoxyphenyl)methoxy]phenyl]-2-ethylpyrazole-3-carboxylate (1.1 g, 2.509 mmol, 1 equiv) in THF (10 mL) and H₂O (10 mL) was added LiOH (0.18 g, 7.527 mmol, 3 equiv) in portions at room temperature. The resulting mixture was stirred for 1 h at room temperature. The mixture acidified to pH ˜5 with HCl (2 M). The precipitated solids were collected by filtration and washed with water (3×30 mL). This resulted in 5-[2-(1,3-dioxolan-2-yl)-3-[(4-methoxyphenyl)methoxy]phenyl]-2-ethylpyrazole-3-carboxylic acid (800.0 mg, 66.30%) as a yellow solid. MS (ESI) calcd. for C₂₃H₂₄N₂O₆, 424.16 m/z, found 425.15 [M+H]⁺.

### Step 4: Synthesis of (1R,3S)-3-(3-(3-(2-(1,3-dioxolan-2-yl)-3-((4-methoxybenzyl)oxy)phenyl)-1-ethyl-1H-pyrazole-5-carboxamido)-1H-pyrazol-5-yl)cyclopentyl isopropylcarbamate

To a stirred solution of methyl 5-[2-(1,3-dioxolan-2-yl)-3-[(4-methoxyphenyl)methoxy]phenyl]-2-ethylpyrazole-3-carboxylic acid (168.2 mg, 0.396 mmol, 1 equiv) and (1R,3S)-3-(5-amino-2H-pyrazol-3-yl)cyclopentyl N-isopropylcarbamate (100.0 mg, 0.396 mmol, 1 equiv) in Pyridine (4 mL) was added EDCI (152.0 mg, 0.792 mmol, 2 equiv) in portions at room temperature. The resulting mixture was stirred for 1 h at room temperature. The reaction mixture was then treated with H₂O (10 mL), extracted with EtOAc (10 mL×2), and the combined extracts washed with brine (10 mL), dried over anhydrous Na₂SO₄ filtered, and concentrated to dryness in vacuo to give a nearly yellow viscous oil. The oil was then subjected to silica gel chromatography (0-30% MeOH/DCM) to give (1R,3S)-3-(3-(3-(2-(1,3-dioxolan-2-yl)-3-((4-methoxybenzyl)oxy)phenyl)-1-ethyl-1H-pyrazole-5-carboxamido)-1H-pyrazol-5-yl)cyclopentyl isopropylcarbamate (140.0 mg, 43.52%) as a yellow solid. MS (ESI) calcd. for C₃₅H₄₂N₆O₇, 658.31 m/z, found 659.35 [M+H]⁺.

### Step 5: Synthesis of (1R,3S)-3-{5-[2-ethyl-5-(2-formyl-3-hydroxyphenyl)pyrazole-3-amido]-2H-pyrazol-3-yl}cyclopentyl N-isopropylcarbamate; trifluoroacetic acid

To a stirred solution/mixture of (1R,3S)-3-(5-{5-[2-(1,3-dioxolan-2-yl)-3-[(4-methoxyphenyl) methoxy]phenyl]-2-ethylpyrazole-3-amido}-2H-pyrazol-3-yl)cyclopentyl N-isopropylcarbamate (100.0 mg, 0.152 mmol, 1 equiv) in DCM (3 mL) was added TFA (1 mL) dropwise at room temperature. The resulting mixture was stirred for 1 h at room temperature. The resulting mixture was concentrated under reduced pressure. The crude product was purified by Prep-HPLC with the following conditions (Column: Xselect CSH OBD Column, 30×150 mm, 5 um; Mobile Phase A: Water (0.05% TFA), Mobile Phase B: ACN; Flow rate: 60 mL/min; Gradient: 38% B to 60% B in 10 min; Wave Length: 254 nm/220 nm; RT1 (min): 9) to afford (1R,3S)-3-{5-[2-ethyl-5-(2-formyl-3-hydroxyphenyl)pyrazole-3-amido]-2H-pyrazol-3-yl}cyclopentyl N-isopropyl carbamate; trifluoroacetic acid (12.3 mg, 13.23%) as a white solid. MS (ESI) calcd. for C₂₇H₃₁F₃N₆O₇, 494.23 m/z, found 495.25 [M+H]⁺. ¹H NMR (300 MHz, DMSO-d₆) δ 11.86 (br s, 1H), 10.95 (s, 1H), 10.38 (s, 1H), 7.64-7.69 (m, 1H), 7.50 (s, 1H), 7.17-7.20 (m, 1H), 6.94-7.03 (m, 2H), 6.47 (s, 1H), 4.99-5.05 (m, 1H), 4.57-4.64 (m, 2H), 3.55-3.62 (m, 1H), 3.07-3.12 (m, 1H), 2.46-2.48 (m, 1H), 2.04-2.06 (m, 1H), 1.86-1.93 (m, 1H), 1.63-1.77 (m, 3H), 1.38-1.43 (m, 3H), 1.03-1.05 (m, 6H). ¹⁹F NMR (282 MHz, DMSO-d₆) δ −74.95.

### Example 5.6: Synthesis of Compound 61: (1R,3S)-3-(3-(1-cyclopropyl-3-(2-formyl-3-hydroxy phenyl)-1H-pyrazole-5-carboxamido)-1H-pyrazol-5-yl)cyclopentyl isopropylcarbamate

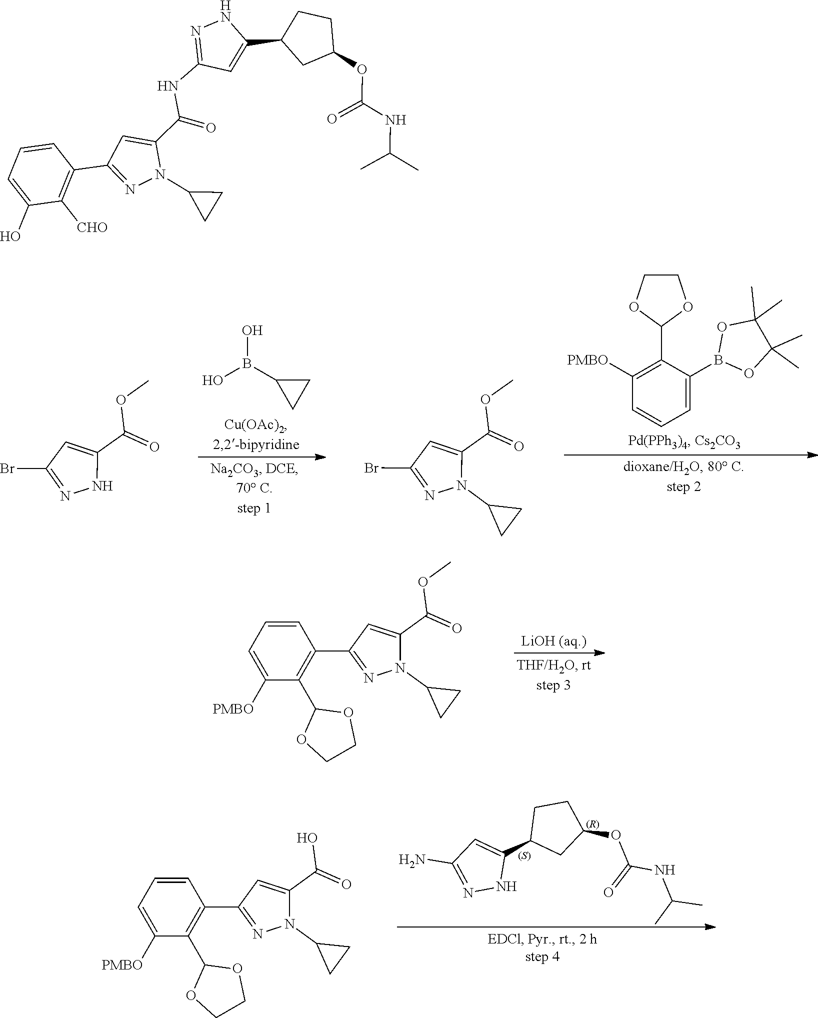

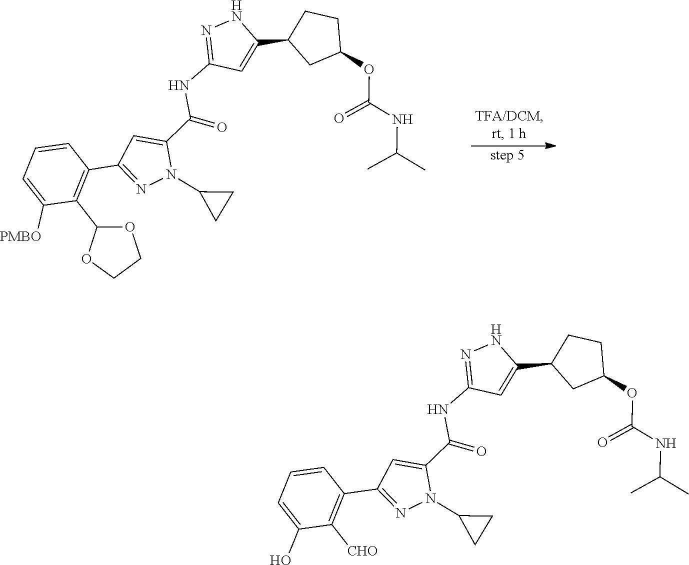

### Step 1: Synthesis of methyl 3-bromo-1-cyclopropyl-1H-pyrazole-5-carboxylate

To a stirred solution of methyl 5-bromo-2H-pyrazole-3-carboxylate (3 g, 14.633 mmol, 1 equiv), cyclopropylboronic acid (3.1 g, 36.582 mmol, 2.5 equiv) and Na₂CO₃ (3.8 g, 36.582 mmol, 2.5 equiv) in DCE (100 mL) were added Cu(OAc)₂ (2.6 g, 14.633 mmol, 1 equiv), 2,2′-bipyridine (2.2 g, 14.633 mmol, 1 equiv) at room temperature. The resulting mixture was stirred for 12 h at 70° C. then cooled to room temperature and diluted with water. The resulting mixture was extracted with EA (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with EA:PE (0-20%) to afford methyl 5-bromo-2-cyclopropylpyrazole-3-carboxylate (1.2 g, 32.62%) as a yellow solid. MS (ESI) calcd. for C₈H₉BrN₂O₂, 243.98 m/z, found 245.00 [M+H]⁺.

### Step 2: Synthesis of methyl 3-(2-(1,3-dioxolan-2-yl)-3-((4-methoxybenzyl)oxy)phenyl)-1-cyclopropyl-1H-pyrazole-5-carboxylate

To a solution of methyl 5-bromo-2-cyclopropylpyrazole-3-carboxylate (1.0 g, 4.080 mmol, 1 equiv) in 1,4-dioxane (15 mL)/H₂O (3 mL) was added 2-[2-(1,3-dioxolan-2-yl)-3-[(4-methoxyphenyl)methoxy]phenyl]-4,4,5,5-tetramethyl-1,3,2-dioxaborolane (2.0 g, 4.896 mmol, 1.2 equiv), Pd(PPh₃)₄ (707.2 mg, 0.612 mmol, 0.15 equiv), Cs₂CO₃ (3.3 g, 10.200 mmol, 2.5 equiv). After stirring for 6 h at 80° C. under N₂ atmosphere, the resulting mixture was cooled to room temperature, diluted with water, and extracted with EA (3×100 mL). The combined organic layers were washed with brine (100 mL), dried over anhydrous Na₂SO₄. After filtration, the filtrate was concentrated under reduced pressure. The crude product was purified by silica gel column chromatography, eluted with EA:PE (1:3) to afford methyl 2-cyclopropyl-5-[2-(1,3-dioxolan-2-yl)-3-[(4-methoxyphenyl)methoxy]phenyl]pyrazole-3-carboxylate (700.0 mg, 26.82%) as a light yellow solid. MS (ESI) calcd. for C₂₅H₂₆N₂O₆, 450.18 m/z, found 451.15 [M+H]⁺.

### Step 3: Synthesis of 3-(2-(1,3-dioxolan-2-yl)-3-((4-methoxybenzyl)oxy)phenyl)-1-cyclopropyl-1H-pyrazole-5-carboxylic acid

To a stirred solution of methyl 2-cyclopropyl-5-[2-(1,3-dioxolan-2-yl)-3-[(4-methoxyphenyl) methoxy]phenyl]pyrazole-3-carboxylate (350.0 mg, 0.777 mmol, 1 equiv) in THF (3 mL) and H₂O (3 mL) was added LiOH (55.8 mg, 2.331 mmol, 3 equiv) at room temperature. The resulting mixture was stirred for 1 h. The mixture was acidified to pH 6 with HCl (1 mol/L). The aqueous layer was extracted with EA (3×20 mL). The resulting mixture was concentrated under reduced pressure to afford 2-cyclopropyl-5-[2-(1,3-dioxolan-2-yl)-3-[(4-methoxyphenyl)methoxy]phenyl]pyrazole-3-carboxylic acid (300.0 mg, 75.82%) as yellow solid. MS (ESI) calcd. for C₂₄H₂₄N₂O₆, 436.16 m/z, found 437.10 [M+H]⁺.

### Step 4: Synthesis of (1R,3S)-3-(3-(3-(2-(1,3-dioxolan-2-yl)-3-((4-methoxybenzyl)oxy)phenyl)-1-cyclopropyl-1H-pyrazole-5-carboxamido)-1H-pyrazol-5-yl)cyclopentyl isopropylcarbamate

To a solution of 2-cyclopropyl-5-[2-(1,3-dioxolan-2-yl)-3-[(4-methoxyphenyl)methoxy]phenyl]pyrazole-3-carboxylic acid (269.8 mg, 0.619 mmol, 1.30 equiv) in pyridine (5 mL) was added (1R,3S)-3-(5-amino-2H-pyrazol-3-yl)cyclopentyl N-isopropylcarbamate (120.0 mg, 0.476 mmol, 1.00 equiv), EDCI (136.7 mg, 0.714 mmol, 1.50 equiv). After stirring for 2 h at room temperature, the resulting mixture was diluted with water and extracted with EA (3×20 mL). The combined organic layers were washed with brine (10 mL), dried over anhydrous Na₂SO₄. After filtration, the filtrate was concentrated under reduced pressure. The crude product was purified by silica gel column chromatography, eluted with EA:PE (1:1) to afford (1R,3S)-3-(5-{2-cyclopropyl-5-[2-(1,3-dioxolan-2-yl)-3-[(4-methoxyphenyl)methoxy]phenyl]pyrazole-3-amido}-2H-pyrazol-3-yl)cyclopentyl N-isopropylcarbamate (100.0 mg, 18.84%) as a yellow solid. MS (ESI) calcd. for C₃₆H₄₂N₆O₇, 670.31 m/z, found 671.25 [M+H]⁺.

### Step 5: Synthesis of (1R,3S)-3-(3-(1-cyclopropyl-3-(2-formyl-3-hydroxyphenyl)-1H-pyrazole-5-carboxamido)-1H-pyrazol-5-yl)cyclopentyl isopropylcarbamate; trifluoroacetic acid

To a stirred solution of (1R,3S)-3-(5-{2-cyclopropyl-5-[2-(1,3-dioxolan-2-yl)-3-[(4-methoxyphenyl)methoxy]phenyl]pyrazole-3-amido}-2H-pyrazol-3-yl)cyclopentyl N-isopropyl-carbamate (100.0 mg, 0.149 mmol, 1 equiv) in DCM (1 mL) were added TFA (0.25 mL) at room temperature. The resulting mixture was stirred for 1 h at room temperature. The reaction progress was monitored by LCMS. The resulting mixture was concentrated under reduced pressure. The crude product was purified by Prep-HPLC with the following conditions: (Column: X Bridge Shield RP18 OBD Column, 30×150 mm, 5 um; Mobile Phase A: Water (0.05% TFA), Mobile Phase B: ACN; Flow rate: 60 mL/min; Gradient: 38% B to 60% B in 10 min; Wave Length: 254 nm/220 nm; RT1 (min): 9) to afford (1R,3S)-3-{5-[2-cyclopropyl-5-(2-formyl-3-hydroxy phenyl)pyrazole-3-amido]-2H-pyrazol-3-yl}cyclopentyl N-isopropylcarbamate; trifluoroacetic acid (6.8 mg, 7.28%) as a light yellow solid. MS (ESI) calcd. for C₂₆H₃₀N₆O₅, 506.23 m/z, found 507.11 [M+H]⁺. ¹H NMR (300 MHz, DMSO-d₆) δ (ppm): 11.83 (s, 1H), 10.95 (s, 1H), 10.31 (s, 1H), 7.62-7.67 (m, 1H), 7.43 (s, 1H), 7.15-7.17 (m, 1H), 6.95-7.02 (m, 2H), 6.48 (s, 1H), 5.02 (s, 1H), 4.48-4.54 (m, 1H), 3.53-3.62 (m, 1H), 3.07-3.12 (m, 1H), 2.53-2.57 (m, 1H), 2.01-2.09 (m, 1H), 1.85-1.97 (m, 1H), 1.61-1.76 (m, 3H), 1.11-1.23 (m, 2H), 1.03-1.05 (m, 8H). ¹⁹F NMR (282 MHz, DMSO-d₆) δ (ppm): −74.961.

### Example 5.7: Synthesis of Compound 65: 3-fluoro-2-hydroxy-5-(2-((2-methoxy-4-(4-(4-methyl piperazin-1-yl)piperidin-1-yl)phenyl)amino)-4-(phenylamino)pyrimidin-5-yl)benzaldehyde

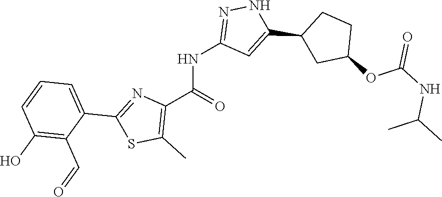 [SMILES: Cc1sc(-c2cccc(O)c2C=O)nc1C(=O)Nc1cc([C@H]2CC[C@@H](OC(=O)NC(C)C)C2)[nH]n1]

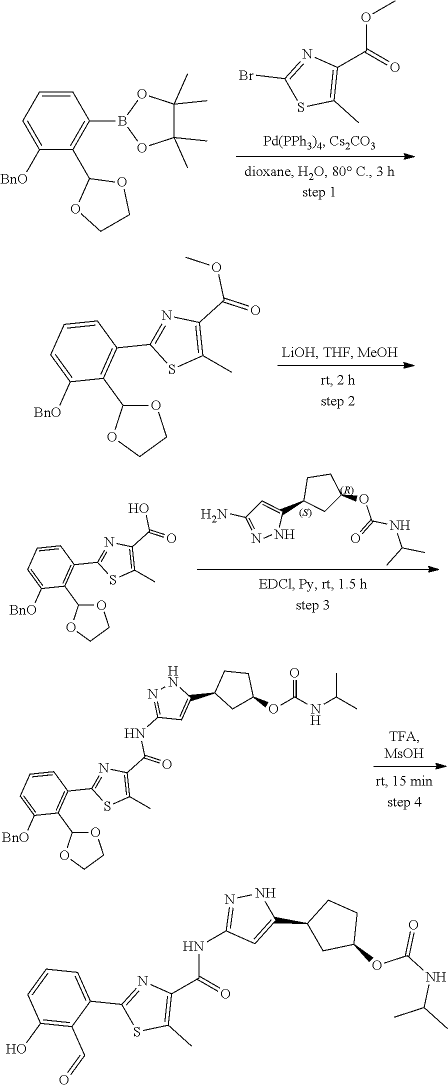

### Step 1: Synthesis of methyl 2-(3-(benzyloxy)-2-(1,3-dioxolan-2-yl)phenyl)-5-methylthiazole-4-carboxylate

Methyl 2-bromo-5-methyl-1,3-thiazole-4-carboxylate (400 mg, 1.694 mmol), 2-[3-(benzyloxy)-2-(1,3-dioxolan-2-yl)phenyl]-4,4,5,5-tetramethyl-1,3,2-dioxaborolane (784 mg, 2.051 mmol), a stir bar, H₂O (1 mL) and dioxane (5 mL) were added to a 40 vial and stirred until homogeneous, then treated with Cs₂CO₃ (1.38 g, 4.222 mmol) and, Pd(PPh₃)₄ (196 mg, 0.170 mmol). The resulting mixture was maintained under nitrogen and stirred at 80° C. for 3 h, then cooled to r.t and diluted with water. The resulting mixture was extracted with EA (2×100 mL). The combined organic layers were washed with brine, dried over anhydrous sodium sulfate, filtered, and concentrated under vacuum. The residue was purified by silica gel chromatography (0-40% EA/PE) to afford methyl 2-[3-(benzyloxy)-2-(1,3-dioxolan-2-yl)phenyl]-5-methyl-1,3-thiazole-4-carboxylate as a light yellow solid (680 mg, 97.54% yield). MS (ESI) mass calcd. for C₂₂H₂₁NO₅S, 411.11 m/z, found, 412.15 [M+H]⁺.

### Step 2: Synthesis of 2-(3-(benzyloxy)-2-(1,3-dioxolan-2-yl)phenyl)-5-methylthiazole-4-carboxylic acid

Methyl 2-[3-(benzyloxy)-2-(1,3-dioxolan-2-yl)phenyl]-5-methyl-1,3-thiazole-4-carboxylate (680 mg, 1.653 mmol), a stir bar, THF (5.1 mL) and MeOH (1.7 mL) were added to a 50 mL round-bottom flask and stirred until homogeneous, and then treated with LiOH (1.7 mL, 5.100 mmol, 3 mol/L). The reaction mixture was stirred for 2 h at r.t, then diluted with water (0.6 mL) and concentrated under vacuum. The pH of the resulting mixture was adjusted to 4-5 with 2 M HCl. The resulting mixture was extracted with EA (100 mL). The combined extracts were washed with brine, dried over anhydrous sodium sulfate, filtered, and concentrated under vacuum to afford 2-[3-(benzyloxy)-2-(1,3-dioxolan-2-yl)phenyl]-5-methyl-1,3-thiazole-4-carboxylic acid as a yellow solid (500 mg, crude). MS (ESI) mass calcd. for C₂₁H₁₉NO₅S, 397.10 m/z, found, 398.10 [M+H]⁺.

### Step 3: Synthesis of (1R,3S)-3-(3-(2-(3-(benzyloxy)-2-(1,3-dioxolan-2-yl)phenyl)-5-methylthiazole-4-carboxamido)-1H-pyrazol-5-yl)cyclopentyl isopropylcarbamate

2-[3-(benzyloxy)-2-(1,3-dioxolan-2-yl)phenyl]-5-methyl-1,3-thiazole-4-carboxylic acid (114 mg, 0.287 mmol), (1R,3S)-3-(5-amino-2H-pyrazol-3-yl)cyclopentyl N-isopropylcarbamate (60 mg, 0.238 mmol), a stir bar and, pyridine (2 mL) were added to a 8 mL vial and stirred until homogeneous, then treated with EDCI (80 mg, 0.417 mmol). The reaction mixture was stirred for 1.5 h at r.t and concentrated under vacuum. The residue was purified by reverse-phase chromatography (5-60% ACN/H₂O with 10 mM NH₄HCO₃) to afford (1R,3S)-3-(5-(2-[3-(benzyloxy)-2-(1,3-dioxolan-2-yl)phenyl]-5-methyl-1,3-thiazole-4-amido-2H-pyrazol-3-yl)cyclopentyl N-isopropylcarbamate as yellow oil (40 mg, 26.63%). MS (ESI) mass calcd. for C₃₃H₃₇N₅O₆S, 631.25 m/z, found, 632.30 [M+H]⁺.

### Step 4: Synthesis of (1R,3S)-3-(3-(2-(2-formyl-3-hydroxyphenyl)-5-methylthiazole-4-carboxamido)-1H-pyrazol-5-yl)cyclopentyl isopropylcarbamate

(1R,3S)-3-(5-(2-[3-(benzyloxy)-2-(1,3-dioxolan-2-yl)phenyl]-5-methyl-1,3-thiazole-4-amido-2H-pyrazol-3-yl)cyclopentyl N-isopropylcarbamate (40 mg, 0.063 mmol), a stir bar and TFA (3 mL) were added to a 8 mL vial and stirred until homogeneous, and then treated with MsOH (0.5 mL). The resulting mixture was stirred for 15 min at room temperature, then concentrated under vacuum and purified by Prep-HPLC with (Column: Xselect CSH OBD Column, 30×150 mm, 5 um; Mobile Phase A: Water (0.05% TFA), Mobile Phase B: ACN; Flow rate: 60 mL/min; Gradient: 35% B to 59% B in 9 min; Wave Length: 254 nm/220 nm nm; RT1 (min): 8) to afford (1R,3S)-3-(5-[2-(2-formyl-3-hydroxyphenyl)-5-methyl-1,3-thiazole-4-amido]-2H-pyrazol-3-ylcyclopentyl N-isopropylcarbamate as a light yellow solid (21.6 mg, 68.53% yield, MS (ESI) mass calcd. for C₂₄H₂₇N₅O₅S, 497.17 m/z, found, 498.20 [M+H]⁺). ¹H NMR (400 MHz, DMSO-d₆) δ 11.65 (s, 1H), 10.79 (s, 1H), 10.40 (s, 1H), 7.68 (t, J=8.0 Hz, 1H), 7.30 (d, J=7.5 Hz, 1H), 7.17 (d, J=8.4 Hz, 1H), 7.01-6.89 (m, 1H), 6.40 (s, 1H), 5.01 (s, 1H), 3.65-3.51 (m, 1H), 3.17-3.00 (m, 1H), 2.64 (s, 3H), 2.51-2.44 (m, 1H), 2.08-1.99 (m, 1H), 1.96-1.83 (m, 1H), 1.81-1.68 (m, 2H), 1.62 (s, 1H), 1.04 (d, J=6.6 Hz, 6H).

### Example 5.8: Synthesis of Compound 66: (1R,3S)-3-(5-{2-[5-(2-formyl-3-hydroxyphenyl)-1,3-thiazol-2-yl]acetamido}-2H-pyrazol-3-yl)cyclopentyl N-isopropylcarbamate; trifluoroacetic acid

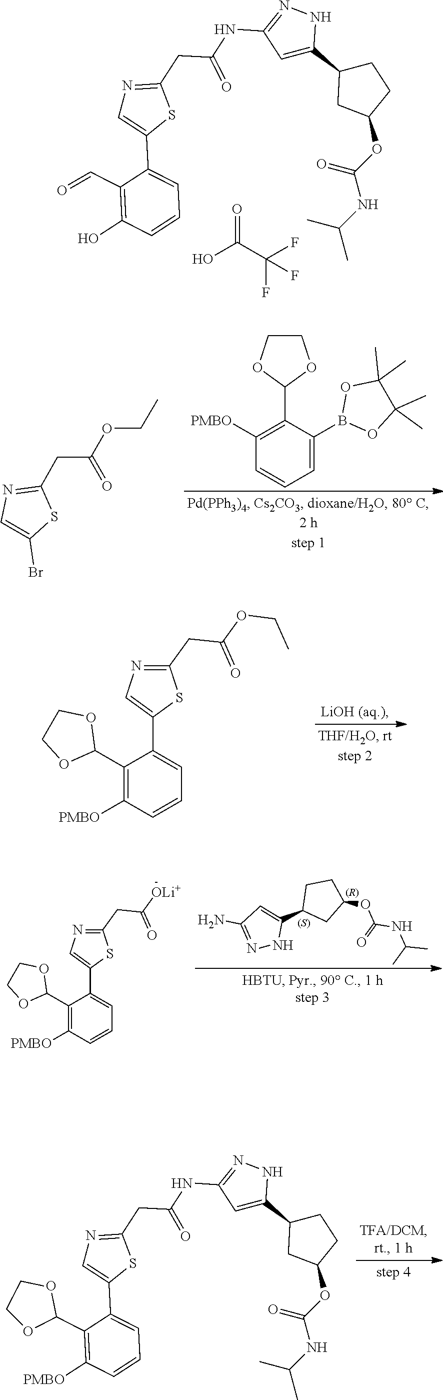

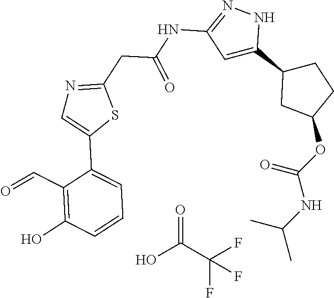

### Step 1: Synthesis of ethyl 2-(5-(2-(1,3-dioxolan-2-yl)-3-((4-methoxybenzyl)oxy)phenyl) thiazol-2-yl)acetate

To a solution of ethyl 2-(5-bromo-1,3-thiazol-2-yl)acetate (400.0 mg, 1.599 mmol, 1 equiv) and 2-[2-(1,3-dioxolan-2-yl)-3-[(4-methoxyphenyl)methoxy]phenyl]-4,4,5,5-tetramethyl-1,3,2-dioxaborolane (1318.8 mg, 3.198 mmol, 2 equiv) in dioxane (8 mL) and H₂O (2 mL) were added Pd(PPh₃)₄ (184.8 mg, 0.160 mmol, 0.1 equiv) and Cs₂CO₃ (1302.7 mg, 3.998 mmol, 2.5 equiv). The resulting mixture was stirred for 2 h at 80° C. The reaction mixture was then treated with H₂O (20 mL), dropwise over 10 min, extracted with EtOAc (20 mL×2), and the combined extracts washed with brine (20 mL), dried over anhydrous Na₂SO₄, filtered, and concentrated to dryness in vacuo to give a nearly yellow viscous oil. The oil was then subjected to silica gel chromatography (0-40% EtOAc/PE) to give ethyl 2-(5-(2-(1,3-dioxolan-2-yl)-3-((4-methoxybenzyl)oxy)phenyl) thiazol-2-yl)acetate (640.0 mg, 72.69%) as a yellow oil. MS (ESI) calcd. for C₂₄H₂₅NO₆S, 455.14 m/z, found 456.25 [M+H]⁺.

### Step 2: Synthesis of lithium 2-(5-(2-(1,3-dioxolan-2-yl)-3-((4-methoxybenzyl)oxy)phenyl) thiazol-2-yl)acetate

To a stirred solution of ethyl 2-{5-[2-(1,3-dioxolan-2-yl)-3-[(4-methoxyphenyl)methoxy]phenyl]-1,3-thiazol-2-yl}acetate (300.0 mg, 0.659 mmol, 1 equiv) in THF (5 mL) and H₂O (5 mL) was added LiOH (47.32 mg, 1.977 mmol, 3 equiv) in portions at room temperature. The resulting mixture was stirred for 1 h at room temperature. The mixture was acidified to pH ˜8 with HCl (1 M). The resulting mixture was lyophilized to give lithium 2-(5-(2-(1,3-dioxolan-2-yl)-3-((4-methoxybenzyl)oxy)phenyl)thiazol-2-yl)acetate (200.0 mg, 63.70%) as a white solid. MS (ESI) calcd. for C₂₂H₂₁NO₆S, 427.11 m/z, found 428.10 [M+H]⁺.

### Step 3: Synthesis of (1R,3S)-3-(3-(2-(5-(2-(1,3-dioxolan-2-yl)-3-((4-methoxybenzyl)oxy) phenyl)thiazol-2-yl)acetamido)-1H-pyrazol-5-yl)cyclopentyl isopropylcarbamate

To a stirred solution of lithium 2-(5-(2-(1,3-dioxolan-2-yl)-3-((4-methoxybenzyl)oxy)phenyl) thiazol-2-yl)acetate (188.9 mg, 0.436 mmol, 1.1 equiv) and (1R,3S)-3-(5-amino-2H-pyrazol-3-yl)cyclopentyl N-isopropylcarbamate (100.0 mg, 0.396 mmol, 1 equiv) in pyridine (4 mL) was added HBTU (180.4 mg, 0.475 mmol, 1.2 equiv) in portions at room temperature. The resulting mixture was stirred for 1 h at room temperature. The reaction mixture was then treated with H₂O (10 mL), dropwise over 10 min, extracted with EtOAc (10 mL×2), and the combined extracts washed with brine (10 mL), dried over anhydrous Na₂SO₄, filtered, and concentrated to dryness in vacuo to give a nearly yellow viscous oil. The oil was then subjected to silica gel chromatography (0-40% EtOAc/PE) to give (1R,3S)-3-(3-(2-(5-(2-(1,3-dioxolan-2-yl)-3-((4-methoxybenzyl)oxy)phenyl)thiazol-2-yl)acetamido)-1H-pyrazol-5-yl)cyclopentyl isopropyl-carbamate (50.0 mg, 16.45%) as a yellow solid. MS (ESI) calcd. for C₃₄H₃₉N₅O₇S, 661.26 m/z, found 662.35 [M+H]⁺.

### Step 4: Synthesis of (1R,3S)-3-(5-{2-[5-(2-formyl-3-hydroxyphenyl)-1,3-thiazol-2-yl]acetamido}-2H-pyrazol-3-yl)cyclopentyl N-isopropylcarbamate; trifluoroacetic acid

To a stirred solution of (1R,3S)-3-[5-(2-{5-[2-(1,3-dioxolan-2-yl)-3-[(4-methoxyphenyl)methoxy]phenyl]-1,3-thiazol-2-yl}acetamido)-2H-pyrazol-3-yl]cyclopentyl N-isopropylcarbamate (50.0 mg, 0.076 mmol, 1 equiv) in DCM (1 mL) was added TFA (0.3 mL) dropwise at room temperature. The resulting mixture was stirred for 1 h at room temperature. The resulting mixture was concentrated under reduced pressure. The crude product was purified by Prep-HPLC with the following conditions (Column: XSelect CSH Fluoro Phenyl 30×150 mm, 5 μm; Mobile Phase A: Water (0.05% TFA), Mobile Phase B: ACN; Flow rate: 60 mL/min; Gradient: 22% B to 44% B in 10 min; Wave Length: 254 nm/220 nm; RT1 (min): 9.8) to afford (1R,3S)-3-(5-{2-[5-(2-formyl-3-hydroxyphenyl)-1,3-thiazol-2-yl]acetamido}-2H-pyrazol-3-yl)cyclopentyl N-isopropylcarbamate; trifluoroacetic acid (7.1 mg, 14.65%) as a light yellow solid. MS (ESI) calcd. for C₂₆H₂₈F₃N₅O₇S, 497.17 m/z, found 498.20 [M+H]⁺. ¹H NMR (400 MHz, DMSO-d₆) δ 11.66 (s, 1H), 10.72-10.76 (m, 1H), 10.06 (s, 1H), 7.84 (s, 1H), 7.59-7.64 (m, 1H), 6.95-7.12 (m, 2H), 6.91-6.93 (m, 1H), 6.33 (s, 1H), 4.97-5.02 (m, 1H), 4.17 (s, 2H), 3.55-3.57 (m, 1H), 3.03-3.07 (m, 1H), 2.44-2.46 (m, 1H), 1.96-2.02 (m, 1H), 1.86-1.91 (m, 1H), 1.68-1.72 (m, 2H), 1.55-1.59 (m, 1H), 1.01-1.03 (m, 6H). ¹⁹F NMR (376 MHz, DMSO-d₆) δ −74.46.

### Example 5.9: Synthesis of Compound 67: (1R,3S)-3-(3-(2-(2-(2-formyl-3-hydroxyphenyl)thiazol-5-yl)acetamido)-1H-pyrazol-5-yl)cyclopentyl isopropylcarbamate; trifluoroacetic acid

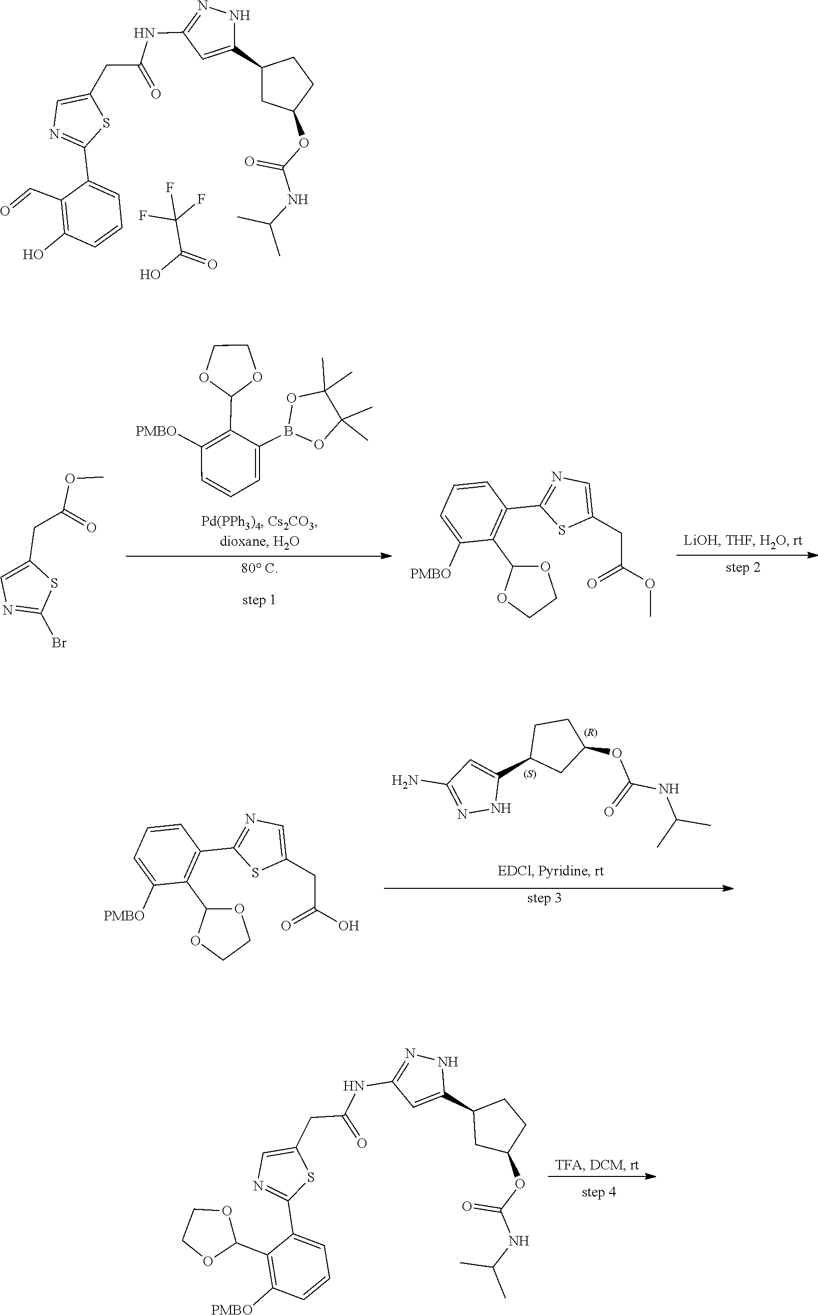

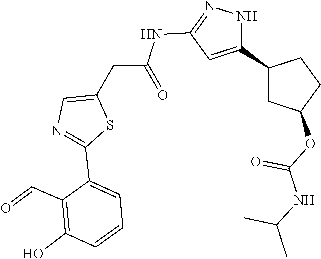 [SMILES: CC(C)NC(=O)O[C@@H]1CC[C@H](c2cc(NC(=O)Cc3cnc(-c4cccc(O)c4C=O)s3)n[nH]2)C1]

### Step 1: Synthesis of methyl 2-{2-[2-(1,3-dioxolan-2-yl)-3-[(4-methoxyphenyl)methoxy]phenyl]-1,3-thiazol-5-yl}acetate

To a stirred solution of methyl 2-(2-bromo-1,3-thiazol-5-yl)acetate (500.0 mg, 2.118 mmol, 1 equiv) and 2-[2-(1,3-dioxolan-2-yl)-3-[(4-methoxyphenyl)methoxy]phenyl]-4,4,5,5-tetramethyl-1,3,2-dioxaborolane (1047.84 mg, 2.542 mmol, 1.2 equiv) in dioxane (10 mL) and H₂O (2 mL) were added Pd(PPh₃)₄ (244.75 mg, 0.212 mmol, 0.1 equiv) and Cs₂CO₃ (2070.18 mg, 6.354 mmol, 3 equiv) at 25° C. The resulting mixture was stirred for 3 h at 80° C. under N₂ atmosphere, then cooled to room temperature and diluted with water. The resulting mixture was extracted with EA (3×30 mL). The combined organic layers were washed with brine (2×30 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O/ACN, 30% to 50% gradient in 10 min; detector, UV 254 nm. This resulted in methyl 2-{2-[2-(1,3-dioxolan-2-yl)-3-[(4-methoxyphenyl)methoxy]phenyl]-1,3-thiazol-5-yl}acetate (250.0 mg, 26.09%) as a yellow oil. MS (ESI) calcd. for C₂₃H₂₃NO₆S, 441.50 m/z, found 442.15 [M+H]⁺.

### Step 2: Synthesis of {2-[2-(1,3-dioxolan-2-yl)-3-[(4-methoxyphenyl)methoxy]phenyl]-1,3-thiazol-5-yl}acetic acid

To a stirred solution of methyl 2-{2-[2-(1,3-dioxolan-2-yl)-3-[(4-methoxyphenyl)methoxy]phenyl]-1,3-thiazol-5-yl}acetate (230.0 mg, 0.521 mmol, 1 equiv) in THF (5 mL) and H₂O (5 mL) were added LiOH (62.38 mg, 2.605 mmol, 5 equiv) at 25° C. The resulting mixture was stirred for 6 h at 25° C. The reaction was monitored by LCMS. The resulting mixture was concentrated under reduced pressure. The mixture was acidified to pH 6 with HCl (1.0 M). The resulting mixture was extracted with EA (3×30 mL). The combined organic layers were washed with brine (2×30 mL), dried over anhydrous Na₂SO₄. After filtration, the filtrate was concentrated under reduced pressure. This resulted in {2-[2-(1,3-dioxolan-2-yl)-3-[(4-methoxyphenyl) methoxy]phenyl]-1,3-thiazol-5-yl}acetic acid (200.0 mg, 79.82%) as a yellow solid. MS (ESI) calcd. for C₂₂H₂₁NO₆S, 427.47 m/z, found 428.10 [M+H]⁺.

### Step 3: Synthesis of (1R,3S)-3-[5-(2-{2-[2-(1,3-dioxolan-2-yl)-3-[(4-methoxyphenyl)methoxy]phenyl]-1,3-thiazol-5-yl}acetamido)-2H-pyrazol-3-yl]cyclopentyl N-isopropylcarbamate

To a stirred solution of {2-[2-(1,3-dioxolan-2-yl)-3-[(4-methoxyphenyl)methoxy]phenyl]-1,3-thiazol-5-yl}acetic acid (150.0 mg, 0.351 mmol, 1 equiv) and (1R,3S)-3-(5-amino-2H-pyrazol-3-yl)cyclopentyl N-isopropylcarbamate (88.54 mg, 0.351 mmol, 1 equiv) in pyridine (5 mL) were added EDCI (100.90 mg, 0.526 mmol, 1.5 equiv) at 25° C. The resulting mixture was stirred for 2 h at 25° C. The resulting mixture was diluted with water and extracted with EA (3×20 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O/ACN, 20% to 50% gradient in 10 min; detector, UV 254 nm. This resulted in (1R,3S)-3-[5-(2-{2-[2-(1,3-dioxolan-2-yl)-3-[(4-methoxyphenyl)methoxy]phenyl]-1,3-thiazol-5-yl}acetamido)-2H-pyrazol-3-yl]cyclopentyl N-isopropyl carbamate (100.0 mg, 33.22%) as a yellow solid. MS (ESI) calcd. for C₃₄H₃₉N₅O₇S, 661.77 m/z, found 662.30 [M+H]⁺.

### Step 4: Synthesis of (1R,3S)-3-(5-{2-[2-(2-formyl-3-hydroxyphenyl)-1,3-thiazol-5-yl]acetamido}-2H-pyrazol-3-yl)cyclopentyl N-isopropylcarbamate; trifluoroacetic acid

To a stirred solution of (1R,3S)-3-[5-(2-{2-[2-(1,3-dioxolan-2-yl)-3-[(4-methoxyphenyl) methoxy]phenyl]-1,3-thiazol-5-yl}acetamido)-2H-pyrazol-3-yl]cyclopentyl N-isopropyl-carbamate (70.0 mg, 0.106 mmol, 1 equiv) in DCM (3 mL) was added TFA (1 mL) dropwise at 25° C. The resulting mixture was stirred for 1.0 h at 25° C. The reaction was monitored by LCMS. The resulting mixture was concentrated under reduced pressure. The crude product was purified by Prep-HPLC with the following conditions (Column: YMC Triart C18 ExRs, 30×150 mm, 5 μm; Mobile Phase A: Water (0.05% TFA), Mobile Phase B: ACN; Flow rate: 60 mL/min; Gradient: 23% B to 46% B in 10 min; Wave Length: 254/220 nm) to afford (1R,3S)-3-(5-{2-[2-(2-formyl-3-hydroxyphenyl)-1,3-thiazol-5-yl]acetamido}-2H-pyrazol-3-yl)cyclopentyl N-isopropylcarbamate; trifluoroacetic acid (9.8 mg, 14.64%) as a light white solid. MS (ESI) calcd. for C₂₆H₂₈F₃N₅O₇S, 497.57 m/z, found 498.25 [M+H]. ¹H NMR (300 MHz, DMSO-d6) δ 11.64 (br s, 1H), 10.71 (s, 1H), 10.35 (s, 1H), 8.20-8.21 (m, 1H), 7.82 (s, 1H), 7.61-7.64 (m, 1H), 7.26-7.28 (m, 1H), 7.10-7.11 (m, 1H), 6.96-6.98 (m, 1H), 6.30 (s, 1H), 4.97-5.01 (m, 1H), 4.00 (s, 2H), 3.52-3.56 (m, 1H), 3.00-3.05 (m, 1H), 2.48-2.50 (m, 1H), 1.90-2.00 (m, 1H), 1.83-1.89 (m, 1H), 1.65-1.71 (m, 2H), 1.56-1.63 (m, 1H), 0.99-1.01 (m, 6H). ¹⁹F NMR (282 MHz, DMSO-d₆) δ −74.47.

### Example 6 Representative Schemes Following General Scheme E

### Example 6.1: Synthesis of Compound 14: 3-(2-formyl-3-hydroxyphenyl)-N-{5-[(1S,3R)-3-[(6-isopropylpyridazin-3-yl)oxy]cyclopentyl]-1H-pyrazol-3-yl}propanamide

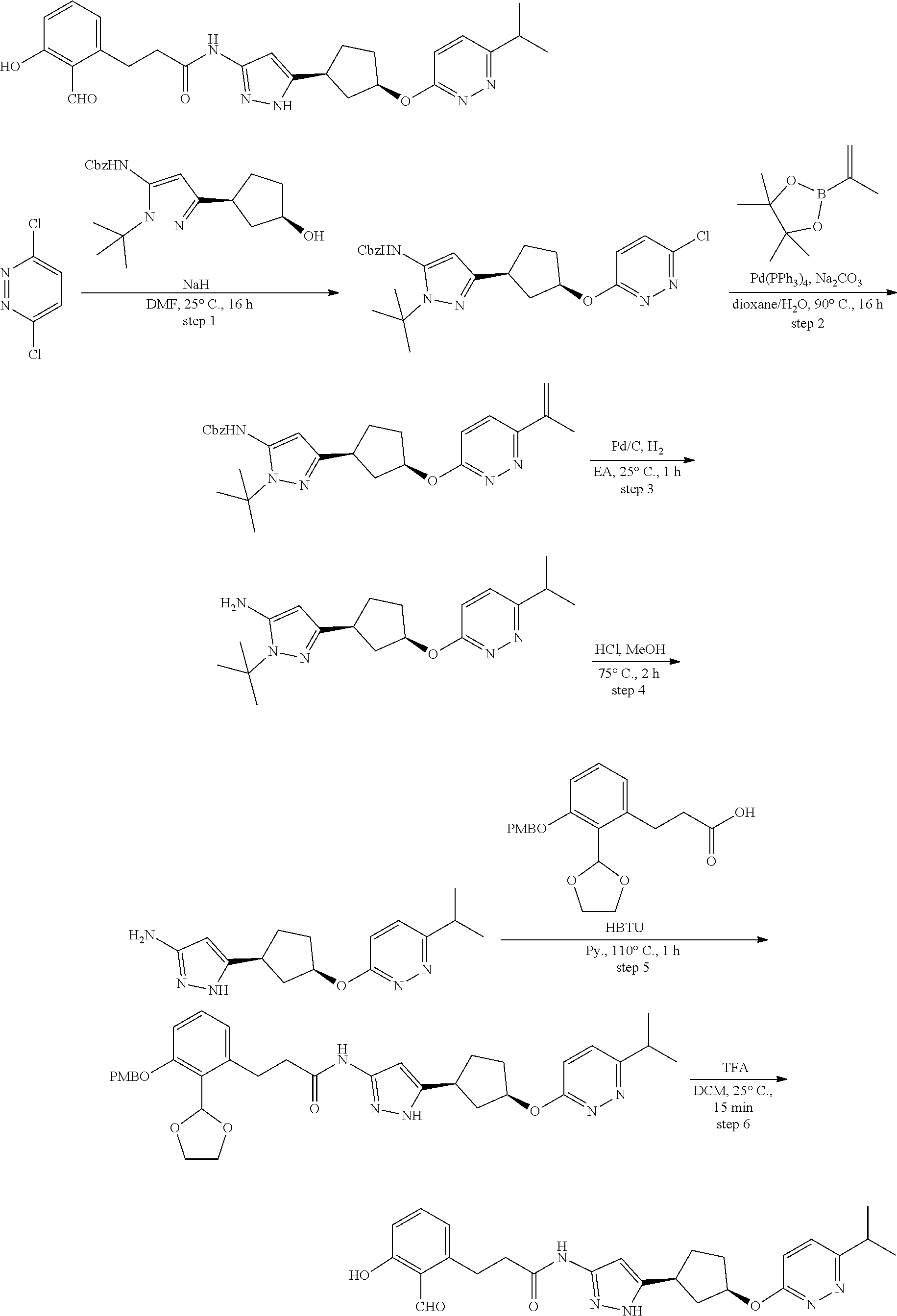

### Step 1: Synthesis of benzyl (1-(tert-butyl)-3-((1S,3R)-3-((6-chloropyridazin-3-yl)oxy)cyclopentyl)-1H-pyrazol-5-yl)carbamate

To a stirred solution of benzyl (1-(tert-butyl)-3-((1S,3R)-3-hydroxycyclopentyl)-1H-pyrazol-5-yl)carbamate (2.4 g, 6.713 mmol, 1 equiv) in DMF (30 mL) was added NaH (805.4 mg, 20.139 mmol, 3 equiv, 60% oil dispersion) at 0° C. under nitrogen atmosphere and stirred for 30 min. Then to the above mixture was added 3,6-dichloropyridazine (2.0 g, 13.426 mmol, 2 equiv) at 0° C. The resulting mixture was stirred for additional 16 h at 25° C. The mixture was diluted with ethyl acetate (70 mL), washed with water (20 mL×3) and brine (40 mL), dried over anhydrous sodium sulfate, and concentrated in vacuo. The residue was purified by silica gel column chromatography, eluted with PE/EA (0-25%) to afford benzyl benzyl (1-(tert-butyl)-3-((1S,3R)-3-((6-chloropyridazin-3-yl)oxy)cyclopentyl)-1H-pyrazol-5-yl)carbamate (3.0 g, 85.20%) as a brown yellow oil. MS (ESI) calcd. for C₂₄H₂₈ClN₅O₃, 469.19 m/z, found 470.15 [M+H]⁺.

### Step 2: Synthesis of benzyl (1-(tert-butyl)-3-((1S,3R)-3-((6-(prop-1-en-2-yl)pyridazin-3-yl)oxy)cyclopentyl)-1H-pyrazol-5-yl)carbamate

To a stirred solution of benzyl (1-(tert-butyl)-3-((1S,3R)-3-((6-chloropyridazin-3-yl)oxy)cyclopentyl)-1H-pyrazol-5-yl)carbamate (1.5 g, 3.192 mmol, 1 equiv) and Na₂CO₃ (1.0 g, 9.576 mmol, 3 equiv) in 1,4-dioxane (15 mL) and H₂O (1.5 mL) were added 4,4,5,5-tetramethyl-2-(prop-1-en-2-yl)-1,3,2-dioxaborolane (2.1 g, 12.768 mmol, 4 equiv) and Pd(dppf)Cl₂ (233.5 mg, 0.319 mmol, 0.1 equiv), the mixture was stirred at 90° C. under nitrogen atmosphere for 16 h. The mixture was diluted with ethyl acetate (70 mL), washed with water (20 mL×3) and brine (40 mL), dried over anhydrous sodium sulfate, and concentrated in vacuum. The residue was purified by silica gel column chromatography, eluted with PE/EA (0-19%) to afford benzyl (1-(tert-butyl)-3-((1S,3R)-3-((6-(prop-1-en-2-yl)pyridazin-3-yl)oxy)cyclopentyl)-1H-pyrazol-5-yl)carbamate (1.1 g, 69.39%) as a brown solid. MS (ESI) calcd. for C₂₇H₃₃N₅O₃, 475.59 m/z, found 498.20 [M+Na]⁺.

### Step 3: Synthesis of 1-(tert-butyl)-3-((1S,3R)-3-((6-isopropylpyridazin-3-yl)oxy)cyclopentyl)-1H-pyrazol-5-amine

To a stirred solution of benzyl (1-(tert-butyl)-3-((1S,3R)-3-((6-(prop-1-en-2-yl)pyridazin-3-yl)oxy)cyclopentyl)-1H-pyrazol-5-yl)carbamate (500.0 mg, 1.051 mmol, 1 equiv) in EtOAc (10 mL) was added Pd/C (145.5 mg, 1.366 mmol, 1.3 equiv, 10%) at 25° C. under hydrogen atmosphere and stirred for 1 h. The resulting mixture was filtered, the filtrate was concentrated under reduced pressure. The residue was purified by Prep-TLC (MeOH/CH₂Cl₂ 1:15) to afford 1-(tert-butyl)-3-((1S,3R)-3-((6-isopropylpyridazin-3-yl)oxy)cyclopentyl)-1H-pyrazol-5-amine (260.0 mg, 61.42%) as a yellow oil. MS (ESI) calcd. for C₁₉H₂₉N₅O, 343.24 m/z, found 344.15 [M+H]⁺.

### Step 4: Synthesis of 5-[(1S,3R)-3-[(6-isopropylpyridazin-3-yl)oxy]cyclopentyl]-1H-pyrazol-3-amine

To a stirred solution of 1-tert-butyl-5-[(1S,3R)-3-[(6-isopropylpyridazin-3-yl)oxy]cyclopentyl]pyrazol-3-amine (160.0 mg, 0.466 mmol, 1 equiv) in CH₃OH (1 mL) was added 4M HCl in dioxane (1 mL) at 25° C. Then the resulting mixture was stirred for additional 2 h at 75° C. The resulting mixture was concentrated under reduced pressure. The residue was purified by Prep-TLC (MeOH/CH₂Cl₂ 1:20) to afford 5-[(1S,3R)-3-[(6-isopropylpyridazin-3-yl)oxy]cyclopentyl]-1H-pyrazol-3-amine (100.0 mg, 85.05% purity by LCMS, 63.50% yield) as a brown oil. MS (ESI) calcd. for C₁₅H₂₁N₅O, 287.17 m/z, found 288.08 [M+H]⁺

### Step 5: Synthesis of 3-[2-(1,3-dioxolan-2-yl)-3-[(4-methoxyphenyl)methoxy]phenyl]-N-{5-[(1S,3R)-3-[(6-isopropylpyridazin-3-yl)oxy]cyclopentyl]-1H-pyrazol-3-yl}propanamide

To a stirred solution of 5-[(1S,3R)-3-[(6-isopropylpyridazin-3-yl)oxy]cyclopentyl]-1H-pyrazol-3-amine (75.0 mg, 0.261 mmol, 1 equiv) and 3-[2-(1,3-dioxolan-2-yl)-3-[(4-methoxyphenyl) methoxy]phenyl]propanoic acid (121.6 mg, 0.339 mmol, 1.3 equiv) in pyridine (10 mL) was added HBTU (98.9 mg, 0.392 mmol, 1.5 equiv) at 25° C. Then the resulting mixture was stirred for additional 1 h at 110° C. The mixture was diluted with ethyl acetate (40 mL), washed with water (20 mL×3) and brine (10 mL), dried over anhydrous sodium sulfate, and concentrated in vacuum. The residue was purified by Prep-TLC (MeOH/CH₂Cl₂ 1:8) to afford 3-[2-(1,3-dioxolan-2-yl)-3-[(4-methoxyphenyl)methoxy]phenyl]-N-{5-[(1S,3R)-3-[(6-isopropylpyridazin-3-yl)oxy]cyclopentyl]-1H-pyrazol-3-yl}propanamide (40.0 mg, 9.56%) as a brown oil. MS (ESI) calcd. for C₃₅H₄₁N₅O₆, 627.31 m/z, found 628.15 [M+H]⁺

### Step 6: Synthesis of 3-(2-formyl-3-hydroxyphenyl)-N-{5-[(1S,3R)-3-[(6-isopropylpyridazin-3-yl)oxy]cyclopentyl]-1H-pyrazol-3-yl}propanamide

A solution of 3-[2-(1,3-dioxolan-2-yl)-3-[(4-methoxyphenyl)methoxy]phenyl]-N-{5-[(1S,3R)-3-[(6-isopropylpyridazin-3-yl)oxy]cyclopentyl]-1H-pyrazol-3-yl}propanamide (80.0 mg, 0.127 mmol, 1 equiv) in TFA (1 mL)/DCM (3 mL) was stirred at 25° C. for 15 min. Then the crude product was further purified by Prep-HPLC with the following condition: Column: Xselect CSH Prep Fluoro-Phenyl Column, 30×150 mm, 5 μm; Mobile Phase A: Water (0.1% FA), Mobile Phase B: ACN; Flow rate: 60 mL/min; Gradient: 20% B to 50% B in 7 min; Wave Length: 254 nm/220 nm; RT1: 6.1 min, it was afforded 3-(2-formyl-3-hydroxyphenyl)-N-{5-[(1S,3R)-3-[(6-isopropylpyridazin-3-yl)oxy]cyclopentyl]-1H-pyrazol-3-yl}propanamide (3.3 mg, 5.40%) as a colorless oil. MS (ESI) calcd. for C₂₅H₂₉N₅O₄, 463.22 m/z, found 464.20 [M+H]⁺. ¹H NMR (300 MHz, DMSO-d₆) δ 12.08 (s, 1H), 11.37 (s, 1H), 10.44 (s, 1H), 10.22 (s, 1H), 7.56 (d, J=9.1 Hz, 1H), 7.43-7.50 (m, 1H), 7.11 (d, J=9.1 Hz, 1H), 6.73-6.88 (m, 2H), 6.31 (s, 1H), 5.55 (s, 1H), 3.10-3.25 (m, 5H), 2.61-2.75 (m, 3H), 2.07-2.15 (m, 2H), 1.92 (m, 1H), 1.68-1.85 (m, 2H), 1.26 (d, J=6.9 Hz, 7H).

### Example 6.2: Synthesis of Compound 22: (1R,3S)-3-(3-(3-(2-formyl-3-hydroxy-5-methoxyphenyl) propanamido)-1H-pyrazol-5-yl)cyclopentyl isopropylcarbamate 2,2,2-trifluoroacetate

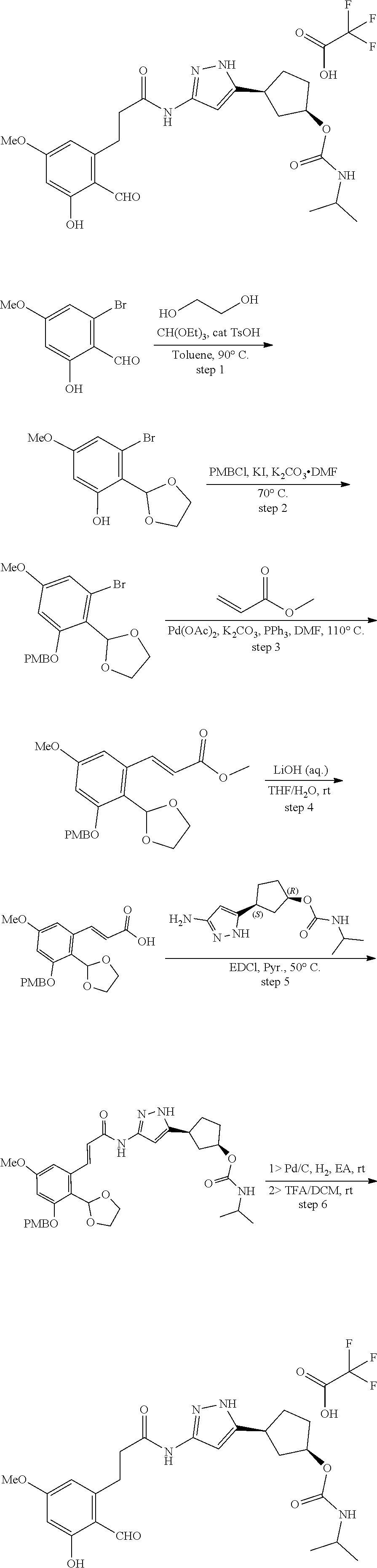

### Step 1: Synthesis of 3-bromo-2-(1,3-dioxolan-2-yl)-5-methoxyphenol

To a stirred solution of 2-bromo-6-hydroxy-4-methoxybenzaldehyde (2.5 g, 10.820 mmol, 1 equiv) in toluene (25 mL) was added triethyl orthoformate (5.40 mL, 32.460 mmol, 3 equiv), para-toluene sulfonate (0.2 g, 1.082 mmol, 0.1 equiv) and ethylene glycol (3.02 mL, 54.100 mmol, 5.0 equiv) at room temperature. The resulting mixture was stirred overnight at 120° C., then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by silica gel column chromatography, eluted with EA:PE (1:5) to afford 3-bromo-2-(1,3-dioxolan-2-yl)-5-methoxyphenol (2.8 g, 94.06%) as light yellow oil. MS (ESI) calcd. for C₁₀H₁₁BrO₄, 273.98 m/z, found 277.00 [M+H]⁺.

### Step 2: Synthesis of 2-{2-bromo-4-methoxy-6-[(4-methoxyphenyl)methoxy]phenyl}-1,3-dioxolane

To a stirred solution of 3-bromo-2-(1,3-dioxolan-2-yl)-5-methoxyphenol (2.5 g, 9.204 mmol, 1 equiv) in DMF (30 mL) was added K₂CO₃ (4.5 g, 32.214 mmol, 3.5 equiv), KI (0.3 g, 1.841 mmol, 0.2 equiv) and PMBCl (1.50 mL, 11.045 mmol, 1.2 equiv) at room temperature. The resulting mixture was stirred overnight at 70° C., then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by silica gel column chromatography, eluted with EA:PE (1:5) to afford 2-{2-bromo-4-methoxy-6-[(4-methoxyphenyl)methoxy]phenyl}-1,3-dioxolane (3.0 g, 82.47%) as a light yellow solid. MS (ESI) calcd. for C₁₈H₁₉BrO₅, 394.04 m/z, found 395.05 [M+H]⁺.

### Step 3: Synthesis of methyl (2E)-3-[2-(1,3-dioxolan-2-yl)-5-methoxy-3-[(4-methoxyphenyl) methoxy]phenyl]prop-2-enoate

To a stirred solution of 2-{2-bromo-4-methoxy-6-[(4-methoxyphenyl)methoxy]phenyl}-1,3-dioxolane (750.0 mg, 1.898 mmol, 1 equiv) in DMF (10 mL) was added Pd(OAc)₂ (85.2 mg, 0.380 mmol, 0.2 equiv), K₂CO₃ (786.8 mg, 5.694 mmol, 3 equiv), PPh₃ (199.1 mg, 0.759 mmol, 0.4 equiv) and methyl acrylate (0.85 mL, 9.490 mmol, 5 equiv) at room temperature under N₂ atmosphere. The resulting mixture was stirred for 2 h at 110° C. under N₂ atmosphere, then cooled to room temperature and diluted with water. The resulting mixture was extracted with EA (3×200 mL). The combined organic layers were washed with brine (3×200 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O, 10% to 50% gradient in 10 min; detector, UV 254 nm. This resulted in methyl (2E)-3-[2-(1,3-dioxolan-2-yl)-5-methoxy-3-[(4-methoxyphenyl)methoxy]phenyl]prop-2-enoate (0.8 g, 98.71%) as yellow oil. MS (ESI) calcd. for C₂₂H₂₄O₇, 400.15 m/z, found 401.15 [M+H]⁺.

### Step 4: Synthesis of (2E)-3-[2-(1,3-dioxolan-2-yl)-5-methoxy-3-[(4-methoxyphenyl)methoxy]phenyl]prop-2-enoic acid

To a stirred solution of methyl (2E)-3-[2-(1,3-dioxolan-2-yl)-5-methoxy-3-[(4-methoxyphenyl) methoxy]phenyl]prop-2-enoate (0.8 g, 1.998 mmol, 1 equiv) in THF (10 mL) was added LiOH (0.1 g, 4.870 mmol, 2.4 equiv) in H₂O (10 mL) at room temperature. The resulting mixture was stirred for 2 h. The mixture was acidified to pH 6 with HCl. The precipitated solids were collected by filtration and washed with H₂O (3×10 mL). This resulted in (2E)-3-[2-(1,3-dioxolan-2-yl)-5-methoxy-3-[(4-methoxyphenyl)methoxy]phenyl]prop-2-enoic acid (577.8 mg, 74.93%) as a white solid. MS (ESI) calcd. for C₂₁H₂₂O₇, 386.14 m/z, found 387.15 [M+H]⁺.

### Step 5: Synthesis of (1R,3S)-3-(3-((E)-3-(2-(1,3-dioxolan-2-yl)-5-methoxy-3-((4-methoxy-benzyl)oxy)phenyl)acrylamido)-1H-pyrazol-5-yl)cyclopentyl isopropylcarbamate

To a stirred solution of (2E)-3-[2-(1,3-dioxolan-2-yl)-5-methoxy-3-[(4-methoxyphenyl)methoxy]phenyl]prop-2-enoic acid (200.0 mg, 0.518 mmol, 1 equiv) and (1R,3S)-3-(5-amino-2H-pyrazol-3-yl)cyclopentyl N-isopropylcarbamate (130.6 mg, 0.518 mmol, 1 equiv) in pyridine (4 mL) were added EDCI (119.1 mg, 0.622 mmol, 1.2 equiv) at room temperature. The resulting mixture was stirred for 2 h at 50° C. then cooled to room temperature and diluted with water. The resulting mixture was extracted with EA (3×50 mL). The combined organic layers were washed with brine (3×50 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in H₂O, 10% to 50% gradient in 10 min; detector, UV 254 nm. This resulted in (1R,3S)-3-{5-[(2E)-3-[2-(1,3-dioxolan-2-yl)-5-methoxy-3-[(4-methoxyphenyl)methoxy]phenyl]prop-2-enamido]-2H-pyrazol-3-yl}cyclopentyl N-isopropylcarbamate (102.0 mg, 31.75%) as a light yellow solid. MS (ESI) calcd. for C₃₃H₄₀N₄O₈, 620.28 m/z, found 621.25 [M+H]⁺.

### Step 6: Synthesis of (1R,3S)-3-{5-[3-(2-formyl-3-hydroxy-5-methoxyphenyl)propanamido]-2H-pyrazol-3-yl}cyclopentyl N-isopropylcarbamate

To a stirred solution of (1R,3S)-3-{5-[(2E)-3-[2-(1,3-dioxolan-2-yl)-5-methoxy-3-[(4-methoxy phenyl)methoxy]phenyl]prop-2-enamido]-2H-pyrazol-3-yl}cyclopentyl N-isopropylcarbamate (90.0 mg, 0.145 mmol, 1 equiv) in EA (4 mL) was added Pd/C (80.0 mg, 10 wt %) at room temperature under H₂ atmosphere for 5 h. The resulting mixture was filtered and concentrated under vacuum. The crude product is directly used for the next step. To the above mixture was added DCM (1.5 mL) and TFA (0.3 mL). The resulting mixture was stirred for 2 h. The crude product was purified by Prep-HPLC with the following conditions (Column: XSelect CSH 5 m, 19 mm×150 mm; Mobile Phase A: Water (0.05% TFA); Mobile Phase B: ACN rate: 25 mL/min; Gradient: 26% B to 56% B in 7 min; Wave Length: 254 nm/220 nm) to afford (1R,3S)-3-{5-[3-(2-formyl-3-hydroxy-5-methoxyphenyl)propanamido]-2H-pyrazol-3-yl}cyclopentyl N-isopropyl carbamate (7.3 mg, 10.63%) as a white solid. MS (ESI) calcd. for C₂₃H₃₀N₄O₆, 458.22 m/z, found 459.20 [M+H]⁺. ¹H NMR (400 MHz, DMSO-d₆) δ 12.14 (br s, 1H), 10.29 (br s, 1H), 10.20 (br s, 1H), 6.94-6.96 (m, 1H), 6.36-6.41 (m, 2H), 6.28 (s, 1H), 4.98-4.99 (m, 1H), 3.79 (s, 3H), 3.56-3.61 (m, 1H), 3.14-3.18 (m, 2H), 3.00-3.05 (m, 1H), 2.56-2.60 (m, 2H), 2.44-2.47 (m, 1H), 1.98-2.04 (m, 1H), 1.86-1.91 (m, 1H), 1.65-1.73 (m, 2H), 1.57-1.60 (m, 1H), 1.03 (d, J=6.4 Hz, 6H). ¹⁹F NMR (376 MHz, DMSO-d₆) δ −74.83.

### Example 6.3: Synthesis of Compound 23: (1R,3S)-3-(3-(3-(2-formyl-3-hydroxyphenyl) propanamido)-1H-pyrazol-5-yl)cyclopentyl (1-methylcyclopropyl)carbamate trifluoroacetic acid

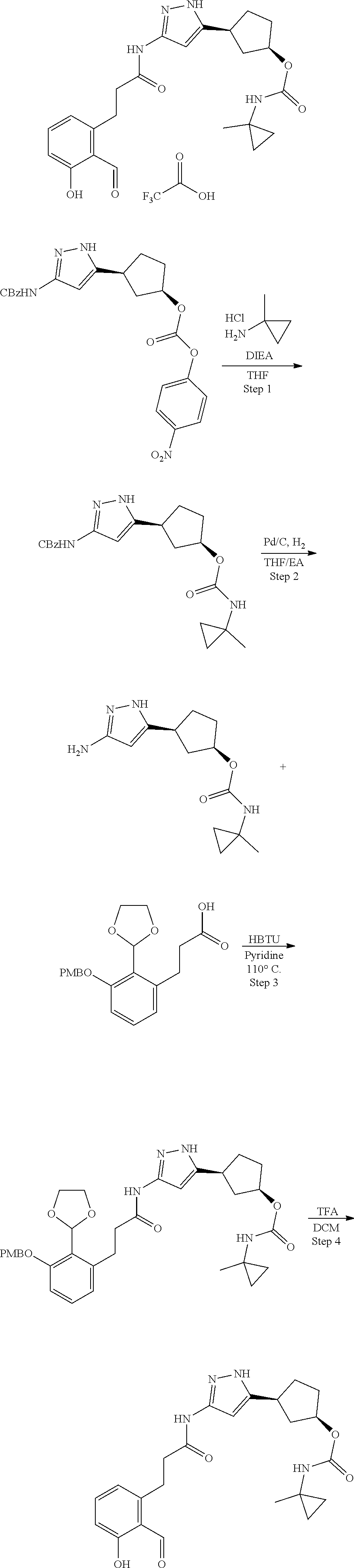

### Step 1: Synthesis of (1R,3S)-3-(3-(((benzyloxy)carbonyl)amino)-1H-pyrazol-5-yl)cyclopentyl (1-methylcyclopropyl)carbamate

To a solution of (1R,3S)-3-(5-{[(benzyloxy)carbonyl]amino}-2H-pyrazol-3-yl)cyclopentyl 4-nitrophenyl carbonate (1.5 g, 3.216 mmol, 1 equiv) in THF (10 mL) was added 1-methylcyclopropan-1-amine hydrochloride (0.69 g, 6.432 mmol, 2 equiv) and DIEA (1.25 g, 9.648 mmol, 3 equiv). After stirring for 10 h at room temperature, the resulting mixture was diluted with water and extracted with EA (3×100 mL). The combined organic layers were washed with NaOH (0.25M, 100 mL), dried over anhydrous Na₂SO₄. After filtration, the filtrate was concentrated under reduced pressure. The crude product was purified by silica gel column chromatography, eluted with EA:PE (1:1) to afford (1R,3S)-3-(5-{[(benzyloxy)carbonyl]amino}-2H-pyrazol-3-yl)cyclopentyl N-(1-methylcyclopropyl)carbamate (700.0 mg, 54.63%) as a light yellow oil. MS (ESI) calcd. for C₂₁H₂₆N₄O₄, 398.20 m/z, found 399.10 [M+H]⁺.

### Step 2: Synthesis of (1R,3S)-3-(3-amino-1H-pyrazol-5-yl)cyclopentyl (1-methylcyclo-propyl)carbamate

To a solution of (1R,3S)-3-(5-{[(benzyloxy)carbonyl]amino}-2H-pyrazol-3-yl)cyclopentyl N-(1-methylcyclopropyl)carbamate (700.0 mg, 1.757 mmol, 1 equiv) in THF/EA (5 mL/5 mL) was added Pd/C (400.0 mg, 0.376 mmol, 0.21 equiv, 10%). After stirring for 3 h at room temperature under an atmosphere of hydrogen, the reaction mixture was filtered through a Celite pad, the filter cake was washed with EA (3×10 mL). The filtrate was concentrated under reduced pressure to yield desired product (1R,3S)-3-(5-amino-2H-pyrazol-3-yl)cyclopentyl N-(1-methylcyclopropyl)carbamate (400.0 mg, 86.14%) as a pink solid. MS (ESI) calcd. for C₁₃H₂₀N₄O₂, 264.16 m/z, found 265.10 [M+H]⁺.

### Step 3: Synthesis of (1R,3S)-3-(3-(3-(2-(1,3-dioxolan-2-yl)-3-((4-methoxybenzyl)oxy)phenyl) propanamido)-1H-pyrazol-5-yl)cyclopentyl (1-methylcyclopropyl)carbamate

A solution of (1R,3S)-3-(5-amino-2H-pyrazol-3-yl)cyclopentyl N-(1-methylcyclopropyl) carbamate (120.0 mg, 0.454 mmol, 1 equiv) in pyridine (10 mL) was added to 3-[2-(1,3-dioxolan-2-yl)-3-[(4-methoxyphenyl)methoxy]phenyl]propanoic acid (162.7 mg, 0.454 mmol, 1 equiv) and HBTU (206.6 mg, 0.545 mmol, 1.2 equiv) and stirred for 5 h at 110° C. After cooling down to rt., the reaction was quenched with water. The resulting mixture was extracted with ethyl acetate (3×10 mL). The organic layers were combined, dried over anhydrous sodium sulfate, filtered and concentrated. The residue obtained was purified by silica gel chromatography (0-90% ethyl acetate/petroleum ether) to afford (1R,3S)-3-(5-{3-[2-(1,3-dioxolan-2-yl)-3-[(4-methoxyphenyl) methoxy]phenyl]propanamido}-2H-pyrazol-3-yl)cyclopentyl N-(1-methylcyclo-propyl) carbamate (70.0 mg, 25.50%) as a brown oil. MS (ESI) calcd. for C₃₃H₄₀N₄O₇, 604.29 m/z, found 605.25 [M+H]⁺.

### Step 4: Synthesis of (1R,3S)-3-(3-(3-(2-formyl-3-hydroxyphenyl)propanamido)-1H-pyrazol-5-yl)cyclopentyl (1-methylcyclopropyl)carbamate

The solution of (1R,3S)-3-(5-{3-[2-(1,3-dioxolan-2-yl)-3-[(4-methoxyphenyl)methoxy]phenyl]propanamido}-2H-pyrazol-3-yl)cyclopentyl N-(1-methylcyclopropyl)carbamate (55.0 mg, 0.091 mmol, 1 equiv) in DCM/TFA (1 mL/0.25) was stirred for 2 h at room temperature. The reaction progress was monitored by LCMS. The resulting mixture was concentrated under reduced pressure. The crude product was purified by Prep-HPLC with the following conditions: (Column: X Bridge Shield RP 18 OBD Column, 30×150 mm, 5 μm; Mobile Phase A: Water (0.05% TFA), Mobile Phase B: ACN; Flow rate: 60 mL/min; Gradient: 26% B to 56% B in 7 min; Wave Length: 254 nm; RT1 (min): 6.31) to afford (1R,3S)-3-{5-[3-(2-formyl-3-hydroxyphenyl)propanamido]-2H-pyrazol-3-yl}cyclopentyl N-(1-methylcyclopropyl)carbamate; trifluoroacetic acid (7.5 mg, 14.75%) as an off-white solid. MS (ESI) calcd. for C₂₃H₂₈N₄O₅, 440.21 m/z, found 441.20 [M+H]⁺. ¹H NMR (400 MHz, DMSO-d₆) δ 11.37 (br s, 1H), 10.45 (s, 1H), 10.29 (s, 1H), 7.38-7.45 (m, 2H), 6.79-6.85 (m, 2H), 6.27 (s, 1H), 4.95-5.01 (m, 1H), 3.19 (t, J=7.6 Hz, 2H), 3.00-3.06 (m, 1H), 2.57 (t, J=7.6 Hz, 2H), 2.42-2.48 (m, 1H), 1.85-2.00 (m, 2H), 1.50-1.74 (m, 3H), 1.24 (s, 3H), 0.45-0.62 (m, 4H).

### Example 6.4: Synthesis of Compound 4: (1R,3S)-3-{5-[2-(4-formyl-3-hydroxyphenyl)acetamido]-2H-pyrazol-3-yl}cyclopentyl N-isopropylcarbamate

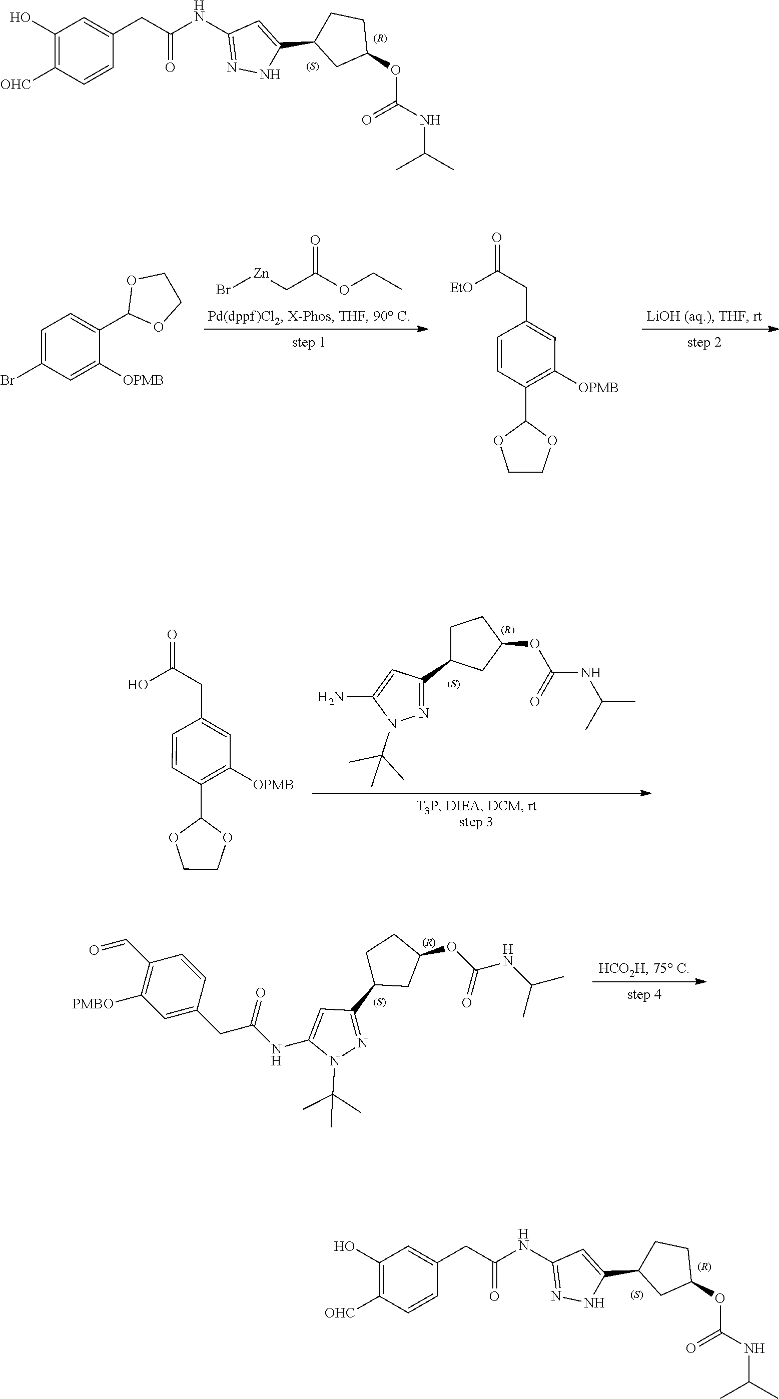

### Step 1: Synthesis of ethyl 2-(4-(1,3-dioxolan-2-yl)-3-((4-methoxybenzyl)oxy)phenyl)acetate

To a stirred solution of 2-{4-bromo-2-[(4-methoxyphenyl)methoxy]phenyl}-1,3-dioxolane (500.0 mg, 1.369 mmol, 1.00 equiv), Pd(dppf)Cl₂ (100.2 mg, 0.137 mmol, 0.1 equiv) and X-Phos (130.5 mg, 0.274 mmol, 0.2 equiv) in THF (10 mL, 123.428 mmol) was added ethyl 2-(bromozincio)acetate (11.41 mL, 6.845 mmol, 5 equiv) at room temperature. The resulting mixture was stirred overnight at 90° C., then cooled to room temperature, diluted with water and extracted with EA (4×50 mL). The combined organic layers were washed with brine (2×50 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O in ACN, 5% to 85% gradient in 30 min; detector, UV 254 nm. This resulted in ethyl 2-[4-(1,3-dioxolan-2-yl)-3-[(4-methoxyphenyl)methoxy]phenyl]acetate (0.25 g, 23.18%) as brown oil. MS (ESI) calcd. for C₂₁H₂₄O₆, 372.16 m/z, found 373.15 [M+H]⁺.

### Step 2: Synthesis of [4-(1,3-dioxolan-2-yl)-3-[(4-methoxyphenyl)methoxy]phenyl]acetic acid

A solution of ethyl 2-[4-(1,3-dioxolan-2-yl)-3-[(4-methoxyphenyl)methoxy]phenyl]acetate (300.0 mg, 0.806 mmol, 1 equiv) in THF (5 mL) was treated with LiOH (57.9 mg, 2.418 mmol, 3 equiv) in H₂O (5 mL) at room temperature. The resulting mixture was stirred for 30 min at room temperature. The mixture was acidified to pH 6 with HCl. The resulting mixture was extracted with DCM (5×50 mL). The combined organic layers were washed with H₂O (2×50 mL), dried over anhydrous Na₂SO₄. After filtration, the filtrate was concentrated under reduced pressure. The resulting mixture was concentrated under vacuum. This resulted in [4-(1,3-dioxolan-2-yl)-3-[(4-methoxyphenyl)methoxy]phenyl]acetic acid (180.0 mg, 48.43%) as brown oil. MS (ESI) calcd. for C₁₉H₂₀O₆, 344.13 m/z, found 343.30 [M−H]⁻.

### Step 3: Synthesis of (1R,3S)-3-[2-tert-butyl-5-(2-{4-formyl-3-[(4-methoxyphenyl)methoxy]phenyl}acetamido)pyrazol-3-yl]cyclopentyl N-isopropylcarbamate

To a stirred mixture of [4-(1,3-dioxolan-2-yl)-3-[(4-methoxyphenyl)methoxy]phenyl]acetic acid (150.0 mg, 0.436 mmol, 1 equiv) and (1R,3S)-3-(5-amino-1-(tert-butyl)-1H-pyrazol-3-yl)cyclopentyl isopropylcarbamate (134.4 mg, 0.436 mmol, 1 equiv) in DCM (10 mL) were added DIEA (168.9 mg, 1.308 mmol, 3 equiv) and T3P (415.8 mg, 1.308 mmol, 3 equiv) dropwise at room temperature. The resulting mixture was stirred for additional 18 h at room temperature. The resulting mixture was extracted with DCM (2×50 mL). The combined organic layers were washed with H₂O (2×50 mL), dried over anhydrous Na₂SO₄. After filtration, the filtrate was concentrated under reduced pressure. The residue was purified by silica gel column chromatography, eluted with DCM:MeOH (5:1) to afford (1R,3S)-3-(1-(tert-butyl)-5-(2-(4-formyl-3-((4-methoxybenzyl) oxy)phenyl)acetamido)-1H-pyrazol-3-yl)cyclopentyl isopropylcarbamate (140.0 mg, 35.45%) as a light yellow solid. MS (ESI) calcd. for C₃₃H₄₂N₄O₈, 590.31 m/z, found 591.40 [M+H]⁺.

### Step 4: Synthesis of (1R,3S)-3-{5-[2-(4-formyl-3-hydroxyphenyl)acetamido]-2H-pyrazol-3-yl}cyclopentyl N-isopropylcarbamate

A solution of (1R,3S)-3-(1-(tert-butyl)-5-(2-(4-formyl-3-((4-methoxybenzyl)oxy)phenyl) acetamido)-1H-pyrazol-3-yl)cyclopentyl isopropylcarbamate (110.0 mg, 0.186 mmol, 1 equiv) in formic acid (5 mL) was stirred for 18 h at 60° C. The resulting mixture was concentrated under reduced pressure. The crude product was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm; RT1 (min): 8.9) to afford (1R,3S)-3-{5-[2-(4-formyl-3-hydroxyphenyl)acetamido]-2H-pyrazol-3-yl}cyclopentyl N-isopropylcarbamate (7.7 mg, 9.87%) as a yellow solid. MS (ESI) calcd. for C₂₁H₂₆N₄O₅, 414.19 m/z, found 415.20 [M+H]⁺. ¹H NMR (300 MHz, DMSO-d₆) δ 12.09 (br s, 1H), 10.59-10.72 (m, 2H), 10.21 (s, 1H), 7.60 (d, J=7.8 Hz, 1H), 6.89-6.96 (m, 3H), 6.29 (s, 1H), 4.97-4.98 (s, 1H), 3.53-3.57 (m, 3H), 3.00-3.05 (m, 1H), 2.40-2.54 (m, 1H), 1.94-2.08 (m, 1H), 1.81-1.91 (m, 1H), 1.63-1.72 (m, 2H), 1.52-1.61 (m, 1H), 1.01-1.24 (m, 6H).

### Example 6.5: Synthesis of Compound 5: (1R,3S)-3-{5-[2-(3-formyl-4-hydroxyphenyl)acetamido]-2H-pyrazol-3-yl}cyclopentyl N-isopropylcarbamate

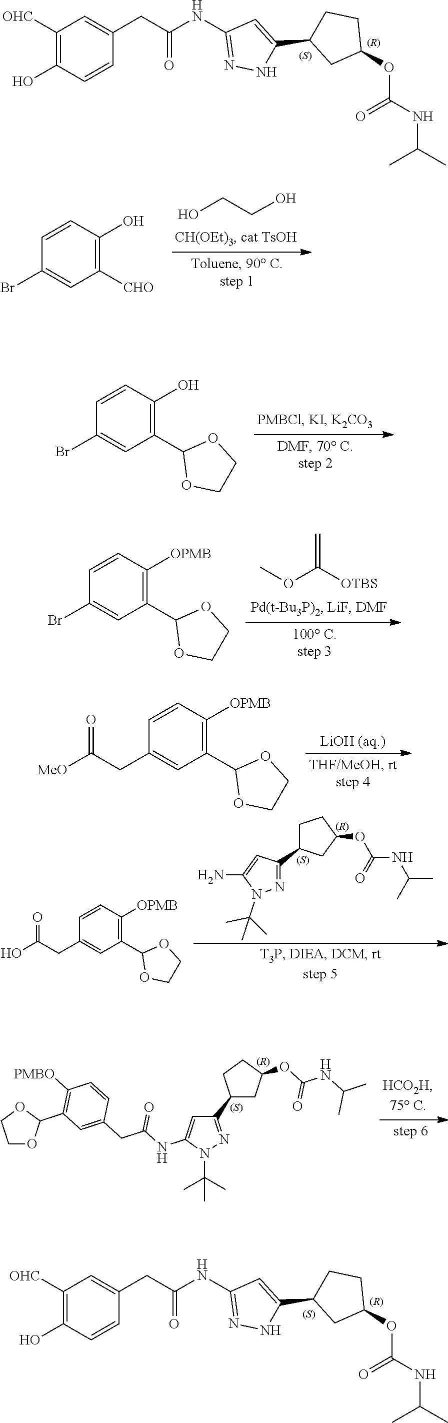

### Step 1: Synthesis of 4-bromo-2-(1,3-dioxolan-2-yl)phenol

To a stirred solution of 5-bromo-2-hydroxybenzaldehyde (20.0 g, 99.493 mmol, 1 equiv) in toluene (300 mL, 9.949 mmol) was added triethyl orthoformate (49.65 mL, 298.479 mmol, 3 equiv), ethylene glycol (27.74 mL, 497.465 mmol, 5 equiv) and TsOH (1.71 g, 9.949 mmol, 0.1 equiv) at room temperature. The resulting mixture was stirred overnight at 90° C. Then cooled to room temperature and diluted with water. The resulting mixture extracted with EA (2×300 mL). The combined organic layers were washed with brine (2×100 mL), dried over anhydrous Na₂SO₄. After filtration, the filtrate was concentrated under reduced pressure. The mixture was purified by silica gel column chromatography, eluted with EA:PE (1:5) to afford 4-bromo-2-(1,3-dioxolan-2-yl)phenol (14.9 g, 61.27%) as alight yellow solid. MS (ESI) calcd. for C₉H₉BrO₃, 243.97 m/z, found 245.05 [M+H]⁺.

### Step 2: Synthesis of 2-{5-bromo-2-[(4-methoxyphenyl)methoxy]phenyl}-1,3-dioxolane

To a stirred solution of 4-bromo-2-(1,3-dioxolan-2-yl) phenol (10.0 g, 40.804 mmol, 1 equiv) in DMF (120 mL) was added K₂CO₃ (28.2 g, 204.020 mmol, 5.0 equiv), KI (1.4 g, 8.161 mmol, 0.2 equiv) and PMBCl (7.7 g, 48.965 mmol, 1.2 equiv) at room temperature. The resulting mixture was stirred overnight at 70° C., then cooled to room temperature and diluted with water. The resulting mixture was extracted with EA (2×300 mL). The combined organic layers were washed with brine (2×100 mL), dried over anhydrous Na₂SO₄. After filtration, the filtrate was concentrated under reduced pressure. The mixture was purified by silica gel column chromatography, eluted with EA:PE (1:5) to afford 2-{5-bromo-2-[(4-methoxyphenyl)methoxy]phenyl}-1,3-dioxolane (13.4 g, 89.70%) as a light yellow solid. MS (ESI) calcd. for C₁₇H₁₇BrO₄, 364.03 m/z, found 365.00 [M+H]⁺.

### Step 3: Synthesis of methyl 2-[2-(1,3-dioxolan-2-yl)-3-[(4-methoxyphenyl)methoxy]phenyl]acetate

To a stirred solution of 2-{5-bromo-2-[(4-methoxyphenyl)methoxy]phenyl}-1,3-dioxolane (1.1 g, 3.012 mmol, 1 equiv), Pd(t-Bu3P)₂ (0.2 g, 0.301 mmol, 0.10 equiv) and LiF (0.2 g, 6.024 mmol, 2 equiv) in DMF (19 mL) were added tert-butyl[(1-methoxyethenyl)oxy]dimethylsilane (2.3 g, 12.048 mmol, 4 equiv) at room temperature. The resulting mixture was stirred for 3 h at 100° C. under N₂ atmosphere. The resulting mixture was filtered, the filter cake was washed with EA (3×200 mL). The filtrate was concentrated under reduced pressure. The residue was purified by reversed-phase flash chromatography with the following conditions: column, C18 silica gel; mobile phase, ACN in H₂O, 10% to 95% gradient in 20 min; detector, UV 254 nm. This resulted in methyl 2-[2-(1,3-dioxolan-2-yl)-3-[(4-methoxyphenyl)methoxy]phenyl]acetate (779.0 mg, 72.17%) as light yellow oil. MS (ESI) calcd. for C₂₀H₂₂O₆, 358.14 m/z, found 359.15 [M+H]⁺.

### Step 4: Synthesis of [2-(1,3-dioxolan-2-yl)-3-[(4-methoxyphenyl)methoxy]phenyl]acetic acid

To a stirred solution of methyl 2-[2-(1,3-dioxolan-2-yl)-3-[(4-methoxyphenyl)methoxy]phenyl]acetate (750.0 mg, 2.093 mmol, 1 equiv) in THF (5 mL) was added LiOH (150.3 mg, 6.279 mmol, 3 equiv) in H₂O (5 mL) at room temperature. The resulting mixture was stirred for 3 h. The mixture was acidified to pH 6 with HCl. The resulting mixture was extracted with EA (2×100 mL). The combined organic layers were washed with brine (2×100 mL), dried over anhydrous Na₂SO₄. After filtration, the filtrate was concentrated under reduced pressure to afford [2-(1,3-dioxolan-2-yl)-3-[(4-methoxyphenyl)methoxy]phenyl]acetic acid (565.0 mg, 78.40%) as a light yellow solid. MS (ESI) calcd. for C₁₉H₂₀O₆, 344.13 m/z, found 342.95 [M−H]⁻.

### Step 5: Synthesis of (1R,3S)-3-(5-(2-(3-(1,3-dioxolan-2-yl)-4-((4-methoxybenzyl)oxy)phenyl) acetamido)-1-(tert-butyl)-1H-pyrazol-3-yl)cyclopentyl isopropylcarbamate

To a stirred solution of [3-(1,3-dioxolan-2-yl)-4-[(4-methoxyphenyl)methoxy]phenyl]acetic acid (170.0 mg, 0.494 mmol, 1 equiv) and (1R,3S)-3-(5-amino-1-(tert-butyl)-1H-pyrazol-3-yl)cyclopentyl isopropylcarbamate (152.2 mg, 0.494 mmol, 1 equiv) in DCM (2 mL) were added DIEA (191.4 mg, 1.482 mmol, 3 equiv) and T3P (471.2 mg, 1.482 mmol, 3 equiv) at room temperature. The resulting mixture was stirred for 1 h then diluted with water (100 mL). The resulting mixture was extracted with DCM (3×50 mL). The combined organic layers were washed with brine (2×50 mL), dried over anhydrous Na₂SO₄. After filtration, the filtrate was concentrated under reduced pressure. The mixture was purified by silica gel column chromatography, eluted with EA:PE (1:5) to afford (1R,3S)-3-(5-(2-(3-(1,3-dioxolan-2-yl)-4-((4-methoxybenzyl)oxy) phenyl)acetamido)-1-(tert-butyl)-1H-pyrazol-3-yl)cyclopentyl isopropylcarbamate (181.0 mg, 42.83%) as a light yellow solid. MS (ESI) calcd. for C₃₅H₄₆N₄O₇, 634.34 m/z, found 635.35 [M+H]⁺.

### Step 6: Synthesis of (1R,3S)-3-{5-[2-(3-formyl-4-hydroxyphenyl)acetamido]-2H-pyrazol-3-yl}cyclopentyl N-isopropylcarbamate

A solution of (1R,3S)-3-(5-(2-(3-(1,3-dioxolan-2-yl)-4-((4-methoxybenzyl)oxy)phenyl) acetamido)-1-(tert-butyl)-1H-pyrazol-3-yl)cyclopentyl isopropylcarbamate (100.0 mg, 0.158 mmol, 1 equiv) in formic acid (3 mL) was stirred for 2 h at 75° C. The resulting mixture was concentrated under reduced pressure. The mixture was purified by reversed-phase flash chromatography with the following conditions: column, C18 silica gel; mobile phase, ACN in H₂O, 5% to 95% gradient in 30 min; detector, UV 254 nm. This resulted in (1R,3S)-3-{5-[2-(3-formyl-4-hydroxyphenyl)acetamido]-2H-pyrazol-3-yl}cyclopentyl N-isopropylcarbamate (7.7 mg, 11.78%) as a light yellow solid. MS (ESI) calcd. for C₂₁H₂₆N₄O₅, 414.19 m/z, found 415.20 [M+H]⁺. ¹H NMR (400 MHz, DMSO-d₆) δ 12.07 (s, 1H), 10.53-10.61 (m, 2H), 10.26 (s, 1H), 7.56-7.60 (m, 1H), 7.43-7.46 (m, 1H), 6.95 (d, J=8.4 Hz, 2H), 6.28 (s, 1H), 4.60-4.97 (m, 1H), 3.46-3.58 (m, 3H), 2.99-3.03 (m, 1H), 2.40-2.47 (m, 1H), 1.91-1.99 (m, 1H), 1.82-1.89 (m, 1H), 1.62-1.70 (m, 2H), 1.45-1.55 (m, 1H), 0.85-1.09 (m, 6H).

### Example 6.6: Synthesis of Compound 6: (1R,3S)-3-{5-[3-(3-formyl-4-hydroxyphenyl)propan-amido]-2H-pyrazol-3-yl}cyclopentyl N-isopropylcarbamate; trifluoroacetic acid

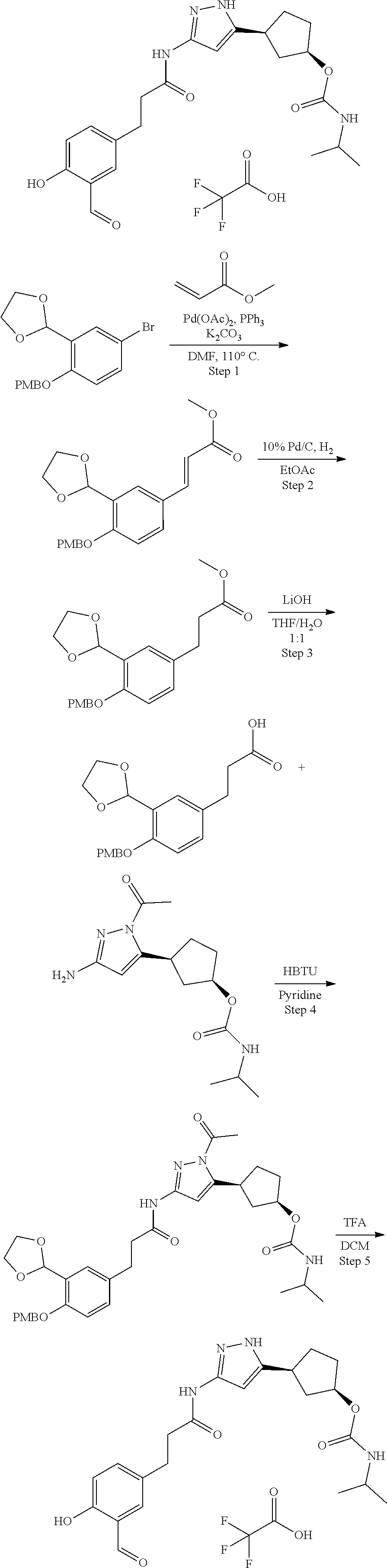

### Step 1: Synthesis of methyl (2E)-3-[3-(1,3-dioxolan-2-yl)-4-[(4-methoxyphenyl)methoxy]phenyl]prop-2-enoate

To a stirred solution of 2-{5-bromo-2-[(4-methoxyphenyl)methoxy]phenyl}-1,3-dioxolane (1.5 g, 4.107 mmol, 1 equiv), Pd(OAc)₂ (138.3 mg, 0.616 mmol, 0.15 equiv), PPh₃ (323.2 mg, 1.232 mmol, 0.3 equiv), K₂CO₃ (1.2 g, 8.214 mmol, 2 equiv) in DMF (15 mL) was added methyl acrylate (1.8 g, 20.535 mmol, 5 equiv) in portions at room temperature under N₂ atmosphere. The resulting mixture was stirred for 2 h at 110° C. The resulting mixture was filtered. The filtrate was purified by reversed-phase flash chromatography with the following conditions: column, C18 silica gel; mobile phase, ACN in H₂O, 5% to 95% gradient in 30 min; detector, UV 254 nm and 220 nm to afford methyl (2E)-3-[3-(1,3-dioxolan-2-yl)-4-[(4-methoxyphenyl)methoxy]phenyl]prop-2-enoate (387.0 mg, 24.93%) as a light yellow solid. MS (ESI) calcd. for C₂₁H₂₂O₆, 370.14 m/z, found 371.10 [M+H]⁺.

### Step 2: Synthesis of methyl 3-(3-(1,3-dioxolan-2-yl)-4-((4-methoxybenzyl)oxy)phenyl) propanoate

To a stirred solution of methyl (2E)-3-[3-(1,3-dioxolan-2-yl)-4-[(4-methoxyphenyl)methoxy]phenyl]prop-2-enoate (300.0 mg, 0.810 mmol, 1 equiv) in EA (10 mL, 102.150 mmol, 126.12 equiv) was added Pd/C (120.0 mg, 10 wt %) at room temperature under H₂ atmosphere. The resulting mixture was stirred for 2 h. The resulting mixture was filtered, the filter cake was washed with EA (3×50 mL). The filtrate was concentrated under reduced pressure. This resulted in methyl 3-(3-(1,3-dioxolan-2-yl)-4-((4-methoxybenzyl)oxy)phenyl) propanoate (195.0 mg, 95.44%) as a light yellow solid. MS (ESI) calcd. For C₂₁H₂₄O₆, 372.16 m/z, found 373.10 [M+H]⁺.

### Step 3: Synthesis of 3-[3-(1,3-dioxolan-2-yl)-4-[(4-ethoxyphenyl)methoxy]phenyl]propanoic acid

To a stirred solution of methyl 3-[3-(1,3-dioxolan-2-yl)-4-[(4-methoxyphenyl)methoxy]phenyl]propanoate (570.0 mg, 1.531 mmol, 1 equiv) in THF (2 mL) was added LiOH (109.9 mg, 4.593 mmol, 3 equiv) in H₂O (2 mL) at room temperature. The resulting mixture was stirred for 2 h at room temperature. The mixture acidified to pH ˜5 with HCl (2 M). The precipitated solids were collected by filtration and washed with water (3×30 mL). This resulted in 3-[3-(1,3-dioxolan-2-yl)-4-[(4-methoxyphenyl)methoxy]phenyl]propanoic acid (450.0 mg, 62.23%) as a white solid. MS (ESI) calcd. for C₂₀H₂₂O₆, 358.14 m/z, found 359.10 [M+H]⁺.

### Step 4: Synthesis of (1R,3S)-3-(3-(3-(3-(1,3-dioxolan-2-yl)-4-((4-methoxybenzyl)oxy)phenyl) propanamido)-1H-pyrazol-5-yl)cyclopentyl isopropylcarbamate

To a stirred solution of 3-[3-(1,3-dioxolan-2-yl)-4-[(4-methoxyphenyl)methoxy]phenyl]propanoic acid (165.7 mg, 0.462 mmol, 1 equiv) and ethyl 5-amino-3-((1S,3R)-3-((isopropylcarbamoyl) oxy)cyclopentyl)-1H-pyrazole-1-carboxylate (150.0 mg, 0.462 mmol, 1.00 equiv) in pyridine (2 mL) were added HBTU (210.4 mg, 0.554 mmol, 1.2 equiv) at room temperature. The resulting mixture was stirred for 2 h at 110° C., then cooled to room temperature and diluted with water. The resulting mixture was extracted with EA (3×50 mL). The combined organic layers were washed with brine (3×50 mL), dried over anhydrous Na₂SO₄. After filtration, the filtrate was concentrated under reduced pressure. This resulted in (1R,3S)-3-(3-(3-(3-(1,3-dioxolan-2-yl)-4-((4-methoxybenzyl)oxy)phenyl)propanamido)-1H-pyrazol-5-yl)cyclopentyl iso-propylcarbamate (100.0 mg, 32.53%) as a yellow solid. MS (ESI) calcd. for C₃₂H₄₀N₄O₇, 592.29 m/z, found 593.30 [M+H]⁺.

### Step 5: Synthesis of (1R,3S)-3-{5-[3-(3-formyl-4-hydroxyphenyl)propanamido]-2H-pyrazol-3-yl}cyclopentyl N-isopropylcarbamate; trifluoroacetic acid

To a stirred solution of (1R,3S)-3-(5-{3-[3-(1,3-dioxolan-2-yl)-4-[(4-methoxyphenyl)methoxy]phenyl]propanamido}-2H-pyrazol-3-yl)cyclopentyl N-isopropylcarbamate (100.0 mg, 0.169 mmol, 1 equiv) in DCM (5 mL) was added TFA (1 mL) dropwise at room temperature. The resulting mixture was stirred for 2 h. The resulting mixture was concentrated under vacuum. The crude product was purified by Prep-HPLC with the following conditions (Column: Xselect CSH F-Phenyl OBD column, 19*250 mm, 5 μm; Mobile Phase A: Water (0.05% TFA), Mobile Phase B: ACN; Flow rate: 25 mL/min; Gradient: 25% B to 55% B in 7 min, 55% B; Wave Length: 254 nm; RT1 (min): 6.23) to afford (1R,3S)-3-{5-[3-(3-formyl-4-hydroxyphenyl)propanamido]-2H-pyrazol-3-yl}cyclopentyl N-isopropylcarbamate (4.9 mg, 6.65%) as a light yellow solid. MS (ESI) calcd. for C₂₂H₂₈N₄O₅, 428.21 m/z, found 429.20 [M+H]⁺. ¹H NMR (400 MHz, DMSO-d₆) δ 10.53 (br s, 1H), 10.28 (br s, 1H), 10.23 (s, 1H), 7.50 (d, J=2.0 Hz, 1H), 7.37-7.40 (m, 1H), 6.96-6.98 (m, 1H), 6.90-6.92 (m, 1H), 6.28 (s, 1H), 4.99 (br s, 1H), 3.54-3.60 (m, 1H), 3.02-3.04 (m, 1H), 2.79-2.83 (m, 2H), 2.54-2.56 (m, 2H), 2.42-2.47 (m, 1H), 1.88-2.00 (m, 1H), 1.85-1.87 (m, 1H), 1.64-1.72 (m, 2H), 1.57-1.59 (m, 1H), 1.03 (d, J=6.4 Hz, 6H). ¹⁹F NMR (376 MHz, DMSO-d₆) δ −74.54.

### Example 6.7: Synthesis of Compound 7: (1R,3S)-3-(3-(3-(2-formyl-3-hydroxyphenyl) propanamido)-1H-pyrazol-5-yl)cyclopentyl isopropylcarbamate trifuoroacetic acid

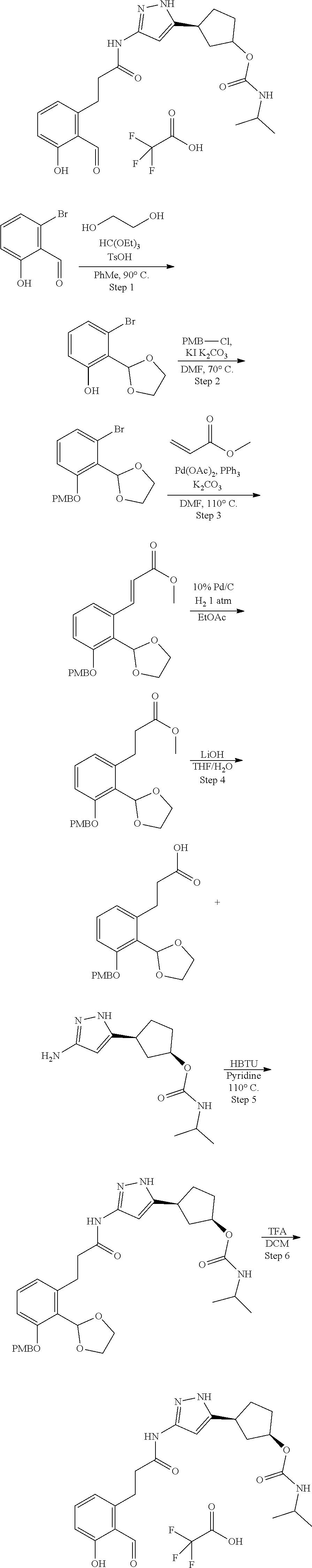

### Step 1: Synthesis of 3-bromo-2-(1,3-dioxolan-2-yl)phenol

To a stirred mixture of 2-bromo-6-hydroxybenzaldehyde (20.0 g, 99.493 mmol, 1 equiv) and Triethyl orthoformate (49.65 mL, 298.479 mmol, 3 equiv) in toluene (300 mL) were added ethylene glycol (30.9 g, 497.465 mmol, 5 equiv) and p-toluenesulfonic acid (1.7 g, 9.949 mmol, 0.1 equiv) dropwise at room temperature. The resulting mixture was stirred for additional 18 h at 90° C., then cooled to room temperature and diluted with water. The resulting mixture was extracted with EA (2×500 mL). The combined organic layers were washed with brine (2×500 mL), dried over anhydrous Na₂SO₄. The residue was purified by silica gel column chromatography, eluted with EA:PE (40%) to afford 3-bromo-2-(1,3-dioxolan-2-yl) phenol (17.3 g, 50.07%) as red oil. MS (ESI) calcd. for C₉H₉BrO₃, 243.97 m/z, found 245.00 [M+H]⁺.

### Step 2: Synthesis of 2-(2-bromo-6-((4-methoxybenzyl)oxy)phenyl)-1,3-dioxolane

To a stirred mixture of 3-bromo-2-(1,3-dioxolan-2-yl)phenol (17.3 g, 70.591 mmol, 1 equiv) and K₂CO₃ (34.2 g, 247.068 mmol, 3.5 equiv) in DMF (200 mL) were added KI (2.3 g, 14.118 mmol, 0.2 equiv) and PMBCl (13.3 g, 84.709 mmol, 1.2 equiv) in portions at room temperature. The resulting mixture was stirred for additional 18 h at 70° C., then cooled to room temperature and diluted with water. The resulting mixture was extracted with EA (2×300 mL). The combined organic layers were washed with brine (2×300 mL), dried over anhydrous Na₂SO₄. After filtration, the filtrate was concentrated under reduced pressure. The residue was purified by silica gel column chromatography, eluted with EA:PE (1:5) to afford 2-{2-bromo-6-[(4-methoxyphenyl)methoxy]phenyl}-1,3-dioxolane (18.0 g, 64.96%) as a pink solid. MS (ESI) calcd. for C₁₇H₁₇BrO₄, 364.03 m/z, found 364.95 [M+H]⁺.

### Step 3: Synthesis of methyl (E)-3-(2-(1,3-dioxolan-2-yl)-3-((4-methoxybenzyl)oxy)phenyl) acrylate

To a stirred solution of 2-{2-bromo-6-[(4-methoxyphenyl)methoxy]phenyl}-1,3-dioxolane (3.0 g, 8.214 mmol, 1 equiv), Pd(OAc)₂ (0.3 g, 1.232 mmol, 0.15 equiv), K₂CO₃ (3.4 g, 24.642 mmol, 3 equiv) and PPh₃ (0.7 g, 2.464 mmol, 0.3 equiv) in DMF (15 mL) was added methyl acrylate (3.5 g, 41.070 mmol, 5 equiv) in portions at room temperature. The resulting mixture was stirred for 1 h at 110° C. under N₂ atmosphere, then cooled to room temperature and diluted with water. The resulting mixture was extracted with EA (2×30 mL). The combined organic layers were washed with brine (2×30 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O in ACN, 5% to 90% gradient in 20 min; detector, UV 254 nm. This resulted in methyl (2E)-3-[2-(1,3-dioxolan-2-yl)-3-[(4-methoxyphenyl)methoxy]phenyl]prop-2-enoate (2.0 g, 65.37%) as a light yellow solid. MS (ESI) calcd. for C₂₁H₂₂O₆, 370.14 m/z, found 371.15 [M+H]⁺.

### Step 4: Synthesis of methyl 3-(2-(1,3-dioxolan-2-yl)-3-((4-methoxybenzyl)oxy)phenyl) propanoate

To a stirred solution of methyl (E)-3-(2-(1,3-dioxolan-2-yl)-3-((4-methoxybenzyl)oxy) phenyl)acrylate (2.0 g, 5.405 mmol, 1 equiv) in EA (40 mL) were added 10% Pd/C (2.0 g) at room temperature. The resulting mixture was stirred for 1 h at room temperature under H₂ atmosphere. The mixture was filtrated, and the filtrate was concentrated under reduced pressure. This resulted in methyl 3-(2-(1,3-dioxolan-2-yl)-3-((4-methoxybenzyl)oxy) phenyl)propanoate (2.0 g, 99.1%) as a white solid. MS (ESI) calcd. for C₂₁H₂₄O₆, 372.16 m/z, found 373.15 [M+H]⁺.

### Step 5: Synthesis of 3-(2-(1,3-dioxolan-2-yl)-3-((4-methoxybenzyl)oxy)phenyl)propanoic acid

A solution of methyl 3-[2-(1,3-dioxolan-2-yl)-3-[(4-methoxyphenyl)methoxy]phenyl]propanoate (2.0 g, 5.370 mmol, 1 equiv) in THF (15 mL) was treated with LiOH (0.3 g, 13.425 mmol, 2.5 equiv) in H₂O (10 mL) at room temperature. The resulting mixture was stirred for 40 min at room temperature. The mixture was acidified to pH 6 with HCl (1 M). The precipitated solids were collected by filtration and washed with water (2×10 mL). This resulted in 3-[2-(1,3-dioxolan-2-yl)-3-[(4-methoxyphenyl)methoxy]phenyl]propanoic acid (1.7 g, 77.18%) as a white solid. MS (ESI) calcd. for C₂₀H₂₂O₆, 358.14 m/z, found 359.15 [M+H]⁺.

### Step 6: Synthesis of (1R,3S)-3-(3-(3-(2-(1,3-dioxolan-2-yl)-3-((4-methoxybenzyl)oxy)phenyl) propanamido)-1H-pyrazol-5-yl)cyclopentyl isopropylcarbamate

To a stirred solution of 3-[2-(1,3-dioxolan-2-yl)-3-[(4-methoxyphenyl)methoxy]phenyl]propanoic acid (100.0 mg, 0.279 mmol, 1 equiv) and (1R,3S)-3-(5-amino-2H-pyrazol-3-yl)cyclopentyl N-isopropylcarbamate (70.4 mg, 0.279 mmol, 1.0 equiv) in pyridine (2 mL) were added HBTU (126.9 mg, 0.335 mmol, 1.2 equiv) at room temperature. The resulting mixture was stirred for 2 h at 110° C. The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE:EA (1:9) to afford (1R,3S)-3-(5-{3-[2-(1,3-dioxolan-2-yl)-3-[(4-methoxyphenyl)methoxy]phenyl]propanamido}-2H-pyrazol-3-yl)cyclopentyl N-isopropyl carbamate (80.0 mg, 48.36%) as a yellow solid. MS (ESI) calcd. for C₃₂H₄₀N₄O₇, 592.29 m/z, found 593.30 [M+H]⁺.

### Step 7: Synthesis of (1R,3S)-3-(3-(3-(2-formyl-3-hydroxyphenyl)propanamido)-1H-pyrazol-5-yl)cyclopentyl isopropylcarbamate; trifluoroacetic acid

To a stirred solution of (1R,3S)-3-(5-{3-[2-(1,3-dioxolan-2-yl)-3-[(4-methoxyphenyl)methoxy]phenyl]propanamido}-2H-pyrazol-3-yl)cyclopentyl N-isopropylcarbamate (80.0 mg, 0.135 mmol, 1 equiv) in DCM (5 mL, 78.653 mmol, 582.71 equiv) was added TFA (1 mL, 13.463 mmol, 99.74 equiv) dropwise at room temperature. The resulting mixture was stirred for 1 h at room temperature. The resulting mixture was concentrated under reduced pressure. The crude product was purified by Prep-HPLC with the following conditions Column: Xcelect CSH F-pheny OBD Column, 19*250 mm, 5 μm; Mobile Phase A: Water (0.05% TFA), Mobile Phase B: ACN; Flow rate: 25 mL/min; Gradient: 32% B to 52% B in 7 min, 52% B; Wave Length: 254 nm; RT1 (min): 6.35 to afford (1R,3S)-3-{5-[3-(2-formyl-3-hydroxyphenyl)propanamido]-2H-pyrazol-3-yl}cyclo pentyl N-isopropylcarbamate (7.4 mg, 12.43%) as a white solid. MS (ESI) calcd. for C₂₂H₂₈N₄O₅, 428.25 m/z, found 429.25 [M+H]⁺. ¹H NMR (400 MHz, DMSO-d6) δ 11.36 (br s, 1H), 10.44 (s, 1H), 10.25 (br s, 1H), 7.41-7.45 (m, 1H), 6.96-6.98 (m, 1H), 6.79-6.84 (m, 2H), 6.28 (s, 1H), 4.95-4.99 (m, 1H), 3.57-3.60 (m, 1H), 3.17-3.21 (m, 2H), 3.00-3.04 (m, 1H), 2.54-2.58 (m, 2H), 2.42-2.47 (m, 1H), 1.96-2.00 (m, 1H), 1.85-1.92 (m, 1H), 1.64-1.72 (m, 2H), 1.53-1.60 (m, 1H), 1.02-1.04 (m, 6H).

### Example 6.8: Synthesis of Compound 12: 1R,3S)-3-(3-(4-(2-formyl-3-hydroxyphenyl) butanamido)-1H-pyrazol-5-yl)cyclopentyl isopropylcarbamate

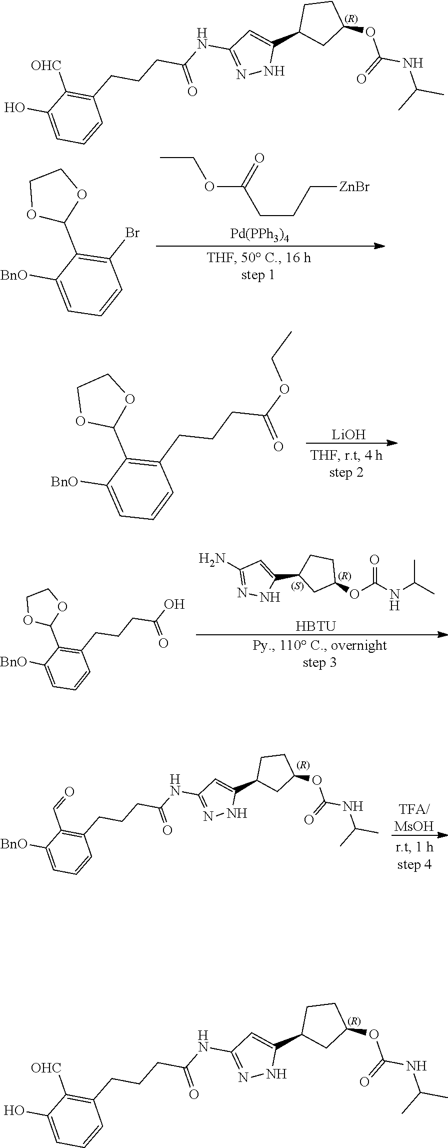

### Step 1: Synthesis of ethyl 4-(3-(benzyloxy)-2-(1,3-dioxolan-2-yl)phenyl)butanoate

To a stirred solution of 2-[2-(benzyloxy)-6-bromophenyl]-1,3-dioxolane (1.0 g, 2.983 mmol, 1 equiv) and Pd(PPh₃)₄ (0.3 g, 0.298 mmol, 0.1 equiv) in THF (10 mL) was added bromo(4-ethoxy-4-oxobutyl)zinc (9.0 mL, 4.474 mmol, 0.5 mol/L, 1.5 equiv) dropwise at room temperature under nitrogen atmosphere. The resulting mixture was stirred for 16 h at 50° C. under nitrogen atmosphere. The reaction was monitored by LCMS. Desired product could be detected by LCMS. The mixture was allowed to cool down to room temperature. The resulting mixture was concentrated under reduced pressure. The residue was purified by silica gel column chromatography, eluted with EA/PE (1:3) to afford ethyl 4-[3-(benzyloxy)-2-(1,3-dioxolan-2-yl)phenyl]butanoate (310 mg, 23.76%) as an off-white solid. MS (ESI) calcd. for C₂₂H₂₆O₅, 370.17 m/z, found 371.15 [M+H]⁺.

### Step 2: Synthesis of 4-(3-(benzyloxy)-2-(1,3-dioxolan-2-yl)phenyl)butanoic acid

To a stirred solution of ethyl 4-[3-(benzyloxy)-2-(1,3-dioxolan-2-yl)phenyl]butanoate (153 mg, 0.413 mmol, 1 equiv) in THF/H₂O (1.5 mL/0.3 mL) was added LiOH·H₂O (34.7 mg, 0.826 mmol, 2 equiv) in portions at room temperature under air atmosphere. The resulting mixture was stirred for 4 h at room temperature under air atmosphere. Desired product could be detected by LCMS. The mixture was neutralized to pH 7 with HCl (2 mol/L). The resulting mixture was extracted with EtOAc (3×10 mL). The combined organic layers were washed with brine (3×10 mL), dried over anhydrous Na₂SO₄. After filtration, the filtrate was concentrated under reduced pressure resulting in 4-[3-(benzyloxy)-2-(1,3-dioxolan-2-yl)phenyl]butanoic acid (187 mg, 83.31%) as a brown yellow oil. MS (ESI) calcd. for C₂₀H₂₂O₅, 342.36 m/z, found 343.00 [M+H]⁺.

### Step 3: Synthesis of (1R,3S)-3-(3-(4-(3-(benzyloxy)-2-formylphenyl)butanamido)-1H-pyrazol-5-yl)cyclopentyl isopropylcarbamate

To a stirred solution of 4-[3-(benzyloxy)-2-(1,3-dioxolan-2-yl)phenyl]butanoic acid (145 mg, 0.423 mmol, 1 equiv) and (1R,3S)-3-(5-amino-2H-pyrazol-3-yl)cyclopentyl N-isopropylcarbamate (106.9 mg, 0.423 mmol, 1 equiv) in pyridine (1 mL) were added HBTU (192.7 mg, 0.508 mmol, 1.2 equiv) in portions at room temperature under air atmosphere. The resulting mixture was stirred for overnight at 110° C. under air atmosphere. The reaction was monitored by LCMS. Desired product could be detected by LCMS. The resulting mixture was diluted with EtOAc (3 mL). The resulting mixture was extracted with H₂O (3×3 mL). The combined organic layers were washed with brine (3×3 mL), dried over anhydrous Na₂SO₄. After filtration, the filtrate was concentrated under reduced pressure. The residue was purified by Prep-TLC (MeOH/CH₂Cl₂=1:20) to afford (1R,3S)-3-(5-{4-[3-(benzyloxy)-2-formylphenyl]butanamido}-2H-pyrazol-3-yl)cyclopentyl N-isopropylcarbamate (23 mg, 7.69%) as a brown yellow oil. MS (ESI) calcd. for C₃₀H₃₆N₄O₅, 532.26 m/z, found 533.30 [M+H]⁺.

### Step 4: Synthesis of (1R,3S)-3-(3-(4-(2-formyl-3-hydroxyphenyl)butanamido)-1H-pyrazol-5-yl)cyclopentyl isopropylcarbamate

A solution of (1R,3S)-3-(5-{4-[3-(benzyloxy)-2-(1,3-dioxolan-2-yl)phenyl]butanamido}-2H-pyrazol-3-yl)cyclopentyl N-isopropylcarbamate (23.0 mg, 0.040 mmol, 1 equiv) in TFA (1 mL) and MsOH (0.2 mL) was stirred for 1 h at room temperature under air atmosphere. Desired product could be detected by LCMS. The crude product was purified by Prep-HPLC with the following conditions (Column: Xselect CSH C18 Column, 19×250 mm, 5 μm; Mobile Phase A: Water (0.1% FA), Mobile Phase B: ACN; Flow rate: 25 mL/min mL/min; Gradient: 35% B to 65% B in 7 min; Wave Length: 254 nm nm; RT1 (min): 6.55) to afford (1R,3S)-3-{5-[4-(2-formyl-3-hydroxyphenyl)butanamido]-2H-pyrazol-3-yl}cyclopentyl N-isopropylcarbamate (1.1 mg, 5.95%) as an off-white solid. MS (ESI) calcd. for C₂₃H₃₀N₄O₅, 442.22 m/z, found 443.15 [M+H]⁺. ¹H NMR (400 MHz, DMSO-d₆) δ 11.41 (s, 1H), 10.42 (s, 1H), 10.28 (s, 1H), 7.52-7.39 (m, 1H), 6.96 (d, J=7.1 Hz, 1H), 6.91-6.88 (m, 1H), 6.87-6.75 (m, 1H), 6.29 (s, 1H), 4.99 (s, 1H), 3.07-2.99 (m, 1H), 2.96-2.88 (m, 2H), 2.50-2.39 (m, 1H), 2.38-2.28 (m, 4H), 2.00 (d, J=8.9 Hz, 1H), 1.93-1.87 (m, 1H), 1.86-1.75 (m, 2H), 1.74-1.61 (m, 2H), 1.58 (s, 1H), 1.03 (d, J=6.5 Hz, 6H).

### Example 6.9: Synthesis of Compound 8: (1R,3S)-3-{5-[1-(2-formyl-3-hydroxyphenyl)pyrazole-4-amido]-2H-pyrazol-3-yl}cyclopentyl N-isopropylcarbamate; trifluoroacetic acid

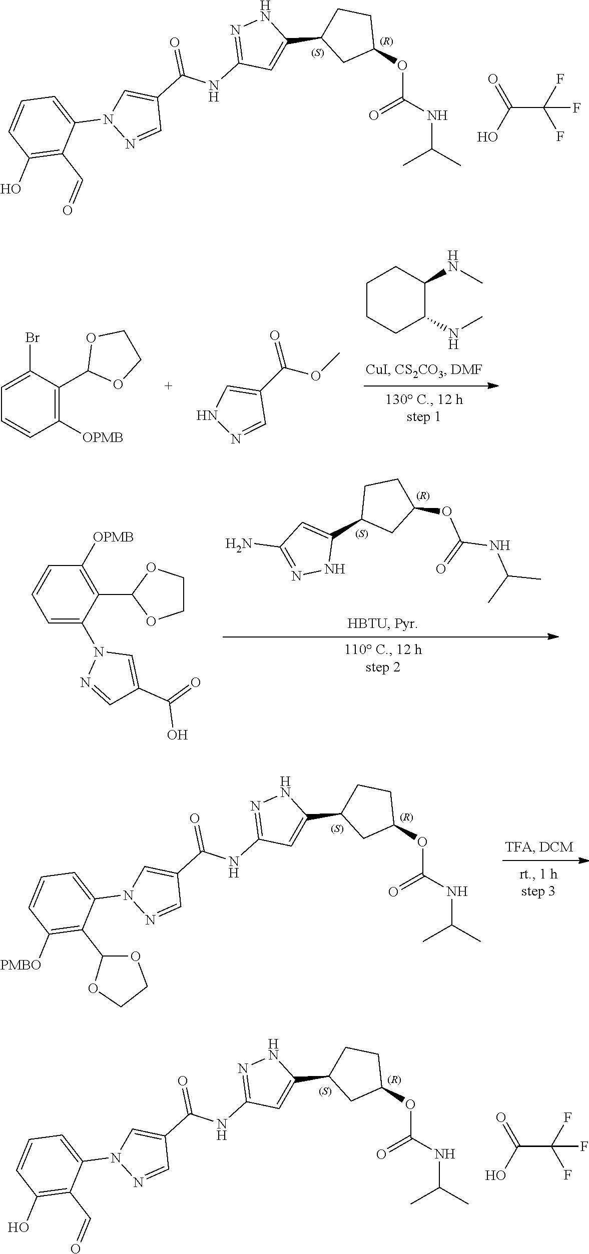

### Step 1: Synthesis of 1-[2-(1,3-dioxolan-2-yl)-3-[(4-methoxyphenyl)methoxy]phenyl]pyrazole-4-carboxylic acid

To a stirred solution of 2-{2-bromo-6-[(4-methoxyphenyl)methoxy]phenyl}-1,3-dioxolane (1.5 g, 4.107 mmol, 1 equiv), methyl 1H-pyrazole-4-carboxylate (517.9 mg, 4.107 mmol, 1 equiv), Cs₂CO₃ (3.35 g, 10.268 mmol, 2.5 equiv) and Trans-(1R,2R)N,N′-Dimethyl-cyclohexane-1,2-diamine (175.0 mg, 1.232 mmol, 0.3 equiv) in DMF (10 mL) was added CuI (78.2 mg, 0.411 mmol, 0.1 equiv) in portions at room temperature under nitrogen atmosphere. The mixture was stirred at 130° C. for 12 h, then cooled to room temperature and diluted with water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (NH₄HCO₃), 45% to 55% gradient in 15 min; detector, UV 254 nm to afford 1-[2-(1,3-dioxolan-2-yl)-3-[(4-methoxyphenyl)methoxy]phenyl]pyrazole-4-carboxylic acid (350.0 mg, 20.85%) as a brown solid. MS(ESI) calcd. for C₂₁H₂₀N₂O₆: 396.13 m/z, found: 397.15 [M+H]⁺.

### Step 2: Synthesis of (1R,3S)-3-(5-{1-[2-(1,3-dioxolan-2-yl)-3-[(4-methoxyphenyl)methoxy]phenyl]pyrazole-4-amido}-2H-pyrazol-3-yl)cyclopentyl N-isopropylcarbamate

To a stirred solution of 1-[2-(1,3-dioxolan-2-yl)-3-[(4-methoxyphenyl)methoxy]phenyl]pyrazole-4-carboxylic acid (243.5 mg, 0.614 mmol, 1.0 equiv) and (1R,3S)-3-(5-amino-2H-pyrazol-3-yl)cyclopentyl N-isopropylcarbamate (155.0 mg, 0.614 mmol, 1.0 equiv) in pyridine (5 mL) was added HBTU (279.6 mg, 0.737 mmol, 1.2 equiv) at room temperature under nitrogen atmosphere. The mixture was stirred at 110° C. for 12 h, then cooled to room temperature and diluted with water. The resulting mixture was extracted with EA (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH (4%˜10%) to afford (1R,3S)-3-(5-{1-[2-(1,3-dioxolan-2-yl)-3-[(4-methoxy phenyl)methoxy]phenyl]pyrazole-4-amido}-2H-pyrazol-3-yl)cyclopentyl N-isopropylcarbamate (142.0 mg, 6.34%) as a brown oil. MS(ESI) calcd. for C₃₃H₃₈N₆O₇: 630.28 m/z, found: 631.30 [M+H]⁺.

### Step 3: Synthesis of (1R,3S)-3-{5-[1-(2-formyl-3-hydroxyphenyl)pyrazole-4-amido]-2H-pyrazol-3-yl}cyclopentyl N-isopropylcarbamate; trifluoroacetic acid

To a stirred solution of (1R,3S)-3-(5-{1-[2-(1,3-dioxolan-2-yl)-3-[(4-methoxyphenyl)methoxy]phenyl]pyrazole-4-amido}-2H-pyrazol-3-yl)cyclopentyl N-isopropylcarbamate (96.0 mg, 0.152 mmol, 1 equiv) in DCM (5 mL) was added TFA (1 mL) dropwise and let stir at room temperature for 1 h. The crude product (50.0 mg) was purified by Prep-HPLC with the following conditions (Column: Xselect CSH F-Phenyl OBD column, 19*250 mm, 5 μm; Mobile Phase A: Water (0.05% TFA), Mobile Phase B: ACN (0.05% TFA); Flow rate: 25 mL/min; Gradient: 30% B to 55% B in 7 min; Wave Length: 254 nm; RT1 (min): 6.32) to afford (1R,3S)-3-{5-[1-(2-formyl-3-hydroxyphenyl)pyrazole-4-amido]-2H-pyrazol-3-yl}cyclopentyl N-isopropylcarbamate; trifluoroacetic acid (6.9 mg, 7.67%) as a light yellow solid. MS (ESI) calcd. for C₂₃H₂₆N₆O₅, 466.20 m/z, found 467.25 [M+H]⁺. ¹H NMR (400 MHz, DMSO-d₆) δ 11.46 (br s, 1H), 10.62 (br s, 1H), 9.94 (s, 1H), 8.87 (s, 1H), 8.35 (s, 1H), 7.66-7.70 (m, 1H), 7.12-7.22 (m, 2H), 6.96-7.10 (m, 1H), 6.43 (s, 1H), 4.95-5.01 (m, 1H), 3.55-3.60 (m, 1H), 3.05-3.09 (m, 1H), 2.03-2.04 (m, 1H), 1.87-2.01 (m, 1H), 1.59-1.77 (m, 4H), 1.03-1.04 (m, 6H). ¹⁹F NMR (376 MHz, DMSO-d₆) δ −74.68.

### Example 6.10: Synthesis of Compound 16: (1R,3S)-3-(5-{2-[1-(2-formyl-3-hydroxyphenyl)pyrazol-4-yl]acetamido}-2H-pyrazol-3-yl)cyclopentyl N-isopropylcarbamate; trifluoroacetic acid

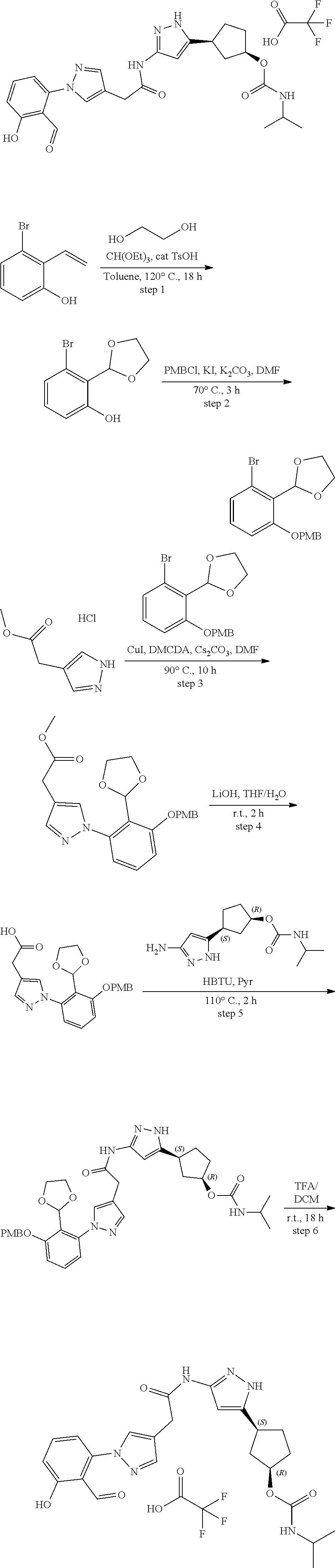

### Step 1: Synthesis of 3-bromo-2-(1,3-dioxolan-2-yl)phenol

To a stirred solution of 2-bromo-6-hydroxybenzaldehyde (30.00 g, 149.240 mmol, 1 equiv) in toluene (600 mL) were added triethyl orthoformate (66.35 g, 447.720 mmol, 3 equiv) ethylene glycol (46.32 g, 746.200 mmol, 5 equiv) and TsOH (77.10 g, 447.720 mmol, 3 equiv) in portions at room temperature. The mixture was stirred at 120° C. for 18 h. The reaction was quenched with Water. The resulting mixture was extracted with EtOAc (3×50 mL). The resulting mixture was washed with water (3×200 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (3:7) to afford 3-bromo-2-(1,3-dioxolan-2-yl)phenol (28.35 g, 62.13%) as a brown oil. MS (ESI) calcd. for C₉H₉BrO₃, 243.97 m/z, found 244.95 [M+H]⁺.

### Step 2: Synthesis of 2-(2-bromo-6-((4-methoxybenzyl)oxy)phenyl)-1,3-dioxolane

To a stirred solution of 3-bromo-2-(1,3-dioxolan-2-yl)phenol (21.00 g, 85.689 mmol, 1 equiv) and K₂CO₃ (35.53 g, 257.067 mmol, 3 equiv) in DMF (300 mL) were added KI (2.84 g, 17.138 mmol, 0.2 equiv) and PMBCl (16.10 g, 102.827 mmol, 1.2 equiv) in portions at room temperature. The mixture was stirred at 70° C. for 3 h. The reaction was quenched with Water. The resulting mixture was extracted with EtOAc (3×50 mL). The resulting mixture was washed with water (3×100 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (1:1) to afford 2-{2-bromo-6-[(4-methoxyphenyl)methoxy]phenyl}-1,3-dioxolane (22.47 g, 59.23%) as a white solid. MS (ESI) calcd. for C₁₇H₁₇BrO₄, 364.03 m/z, found 364.95 [M−H]⁻.

### Step 3: Synthesis of methyl 2-(1-(2-(1,3-dioxolan-2-yl)-3-((4-methoxybenzyl)oxy)phenyl)-1H-pyrazol-4-yl)acetate

To a stirred solution of methyl 2-(1H-pyrazol-4-yl)acetate hydrochloride (400.0 mg, 2.265 mmol, 1 equiv) and 2-{2-bromo-6-[(4-methoxyphenyl)methoxy]phenyl}-1,3-dioxolane (992.7 mg, 2.718 mmol, 1.2 equiv) in DMF (20 mL) were added Cs₂CO₃ (1845.0 mg, 5.663 mmol, 2.5 equiv) and (1S,2S)-1-N,2-N-dimethylcyclohexane-1,2-diamine (96.7 mg, 0.679 mmol, 0.3 equiv) in portions at room temperature under nitrogen atmosphere. The mixture was stirred at 80° C. for 10 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (NH₄HCO₃), 20% to 30% gradient in 10 min; detector, UV 254 nm to afford methyl 2-{1-[2-(1,3-dioxolan-2-yl)-3-[(4-methoxyphenyl) methoxy]phenyl]pyrazol-4-yl}acetate (260.0 mg, 19.79%) as a Brown yellow oil. MS (ESI) calcd. for C₂₃H₂₄N₂O₆, 424.16 m/z, found 425.15 [M+H]⁺.

### Step 4: Synthesis of 2-(1-(2-(1,3-dioxolan-2-yl)-3-((4-methoxybenzyl)oxy)phenyl)-1H-pyrazol-4-yl)acetic acid

To a stirred solution of methyl 2-{1-[2-(1,3-dioxolan-2-yl)-3-[(4-methoxyphenyl)methoxy]phenyl]pyrazol-4-yl}acetate (250.0 mg, 0.589 mmol, 1 equiv) in THF (5 mL) were added H₂O (5 mL) and LiOH (35.3 mg, 1.472 mmol, 2.5 equiv) in portions at room temperature and let stir for 1 h. The mixture was acidified to pH 7 with HCl. The resulting mixture was extracted with EtOAc (3×50 mL). The combined organic layers were washed with brine (2×20 mL), dried over anhydrous Na₂SO₄. After filtration, the filtrate was concentrated under reduced pressure to afford {1-[2-(1,3-dioxolan-2-yl)-3-[(4-methoxyphenyl)methoxy]phenyl]pyrazol-4-yl}acetic acid (213.3 mg, 88.24%) as a yellow semi-solid. MS (ESI) calcd. for C₂₂H₂₂N₂O₆, 410.15 m/z, found 411.10 [M+H]⁺.

### Step 5: Synthesis of (1R,3S)-3-(3-(2-(1-(2-(1,3-dioxolan-2-yl)-3-((4-methoxybenzyl)oxy) phenyl)-1H-pyrazol-4-yl)acetamido)-1H-pyrazol-5-yl)cyclopentyl isopropylcarbamate

To a stirred solution of {1-[2-(1,3-dioxolan-2-yl)-3-[(4-methoxyphenyl)methoxy]phenyl]pyrazol-4-yl}acetic acid (162.7 mg, 0.396 mmol, 1 equiv) and (1R,3S)-3-(5-amino-2H-pyrazol-3-yl)cyclopentyl N-isopropylcarbamate (100.0 mg, 0.396 mmol, 1.00 equiv) in pyridine (2 mL) was added HBTU (225.5 mg, 0.594 mmol, 1.5 equiv) in portions at room temperature. The mixture was stirred at 110° C. for 2 h. The reaction was quenched with Water. The resulting mixture was extracted with EA (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (3:7) to afford (1R,3S)-3-[5-(2-{1-[2-(1,3-dioxolan-2-yl)-3-[(4-methoxyphenyl)methoxy]phenyl]pyrazol-4-yl}acetamido)-2H-pyrazol-3-yl]cyclopentyl N-isopropylcarbamate (146.0 mg, 38.17%) as a Brown yellow solid. MS (ESI) calcd. for C₃₄H₄₀N₆O₇, 644.30 m/z, found 645.20 [M+H]⁺.

### Step 5: Synthesis of (1R,3S)-3-(5-{2-[1-(2-formyl-3-hydroxyphenyl)pyrazol-4-yl]acetamido}-2H-pyrazol-3-yl)cyclopentyl N-isopropylcarbamate; trifluoroacetic acid

To a stirred solution of (1R,3S)-3-[5-(2-{1-[2-(1,3-dioxolan-2-yl)-3-[(4-methoxyphenyl) methoxy]phenyl]pyrazol-4-yl}acetamido)-2H-pyrazol-3-yl]cyclopentyl N-isopropylcarbamate (80.0 mg, 0.124 mmol, 1 equiv) in DCM (2.5 mL) was added TFA (0.8 mL) dropwise at room temperature and let stir for 18 h. The crude product (50.0 mg) was purified by Prep-HPLC with the following conditions (Column: XBridge RP C18 5 m, 19 mm×250 mm; Mobile Phase A: Water (0.05% TFA), Mobile Phase B: ACN; Flow rate: 25 mL/min mL/min; Gradient: 30% B to 50% B in 8 min; Wave Length: 254 nm/220 nm; RT1 (min): 7.59) to afford (1R,3S)-3-(5-{2-[1-(2-formyl-3-hydroxyphenyl)pyrazol-4-yl]acetamido}-2H-pyrazol-3-yl)cyclopentyl N-isopropylcarbamate; trifluoroacetic acid (4.9 mg, 6.43%) as a white solid. MS (ESI) calcd. for C₂₄H₂₈N₆O₅, 480.21 m/z, found 481.20 [M+H]⁺. ¹H NMR (400 MHz, DMSO-d₆) δ 11.46 (br s, 1H), 10.50 (br s, 1H), 9.89 (s, 1H), 8.19 (s, 1H), 7.75 (s, 1H), 7.62-7.66 (m, 1H), 7.08-7.10 (m, 1H), 7.01-7.03 (m, 1H), 6.93-6.98 (m, 1H), 6.31 (br s, 1H), 4.92-5.02 (m, 1H), 3.56-3.73 (m, 3H), 3.01-3.05 (m, 1H), 2.42-2.47 (m, 1H), 1.96-2.00 (m, 1H), 1.83-1.93 (m, 1H), 1.65-1.72 (m, 2H), 1.52-1.60 (m, 1H), 0.95-1.05 (m, 6H). ¹⁹F NMR (376 MHz, DMSO-d₆) δ −74.43.

### Example 6.11: Synthesis of Compound 29: (1R,3S)-3-(5-{5-[2-(2-formyl-3-hydroxyphenyl)ethynyl]-2-methylpyrazole-3-amido}-2H-pyrazol-3-yl)cyclopentyl N-isopropylcarbamate

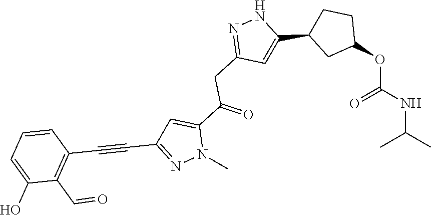

### Step 1: Synthesis of methyl 5-ethynyl-2-methylpyrazole-3-carboxylate

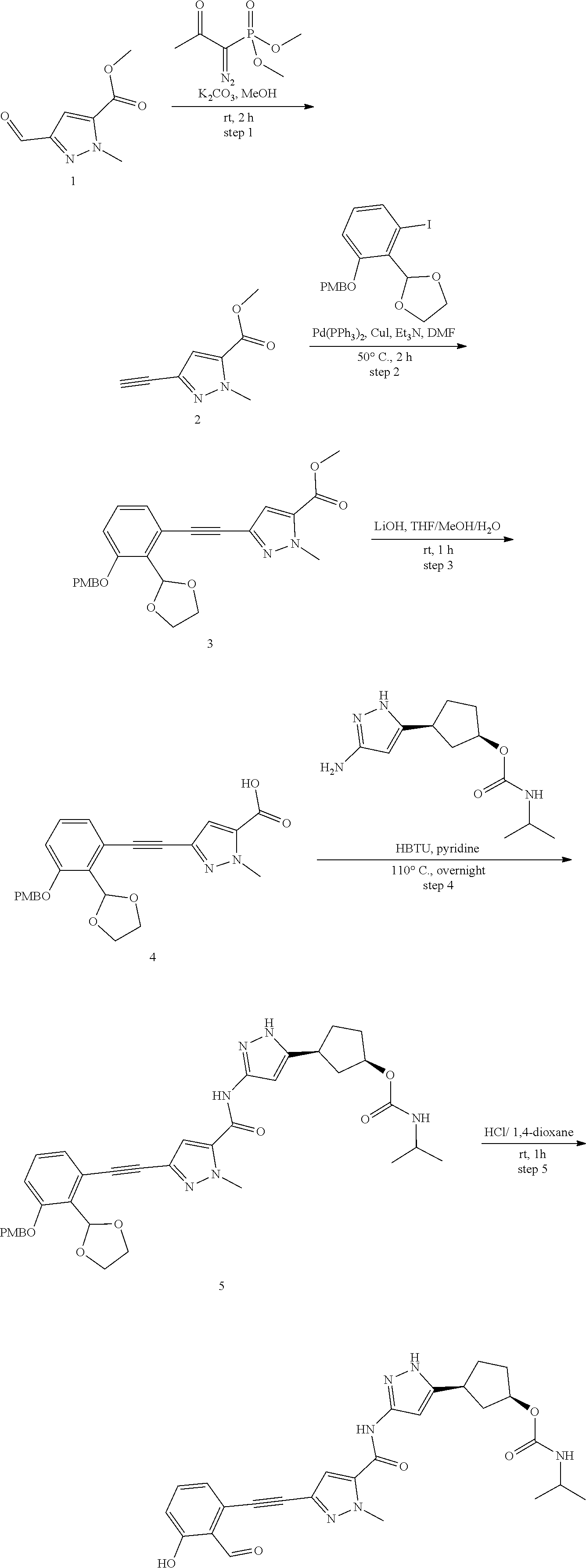

A 100 mL vessel was charged with methyl 5-formyl-2-methylpyrazole-3-carboxylate (2.0 g, 11.894 mmol, 1 equiv), K₂CO₃ (4.9 g, 35.682 mmol, 3 equiv) and MeOH (20 mL). Ethyl 2-diazo-3-oxobutanoate (2.8 g, 17.841 mmol, 1.5 equiv) was added dropwise to the reaction flask at 0° C. and the resulting mixture was stirred at room temperature for 2 h under a nitrogen atmosphere. The reaction mixture was then treated with H₂O (10 mL), extracted with EA (10 mL×3). The combined organic layers were washed with brine (10 mL), dried over anhydrous Na₂SO₄, filtered, and concentrated to dryness in vacuum to give a yellow solid. The yellow solid was then subjected to silica gel chromatography (0-30% PE/EA) to afford methyl 5-ethynyl-2-methylpyrazole-3-carboxylate (1.1 g, 56.3%) as a white solid. MS (ESI), calcd. for C₈H₈N₂O₂, 164.06 m/z, found 165.15 [M+H]⁺.

### Step 2: Synthesis of methyl 5-{2-[2-(1,3-dioxolan-2-yl)-3-[(4-methoxyphenyl)methoxy]phenyl]ethynyl}-2-methylpyrazole-3-carboxylate

Into a 100 mL vessel were added methyl 5-ethynyl-2-methylpyrazole-3-carboxylate (500 mg, 3.046 mmol, 1 equiv), 2-{2-iodo-6-[(4-methoxyphenyl)methoxy]phenyl}-1,3-dioxolane (1255.5 mg, 3.046 mmol, 1 equiv), CuI (116 mg, 0.609 mmol, 0.2 equiv), Pd(PPh₃)₂Cl₂ (213.8 mg, 0.305 mmol, 0.1 equiv) and DMF (10 mL) and the resulting mixture was stirred for 2 h at 50° C. under a nitrogen atmosphere. After cooling down to room temperature, the reaction mixture was treated with H₂O (10 mL), extracted with EA (10 mL×3). The combined organic extracts were washed with brine (10 mL), dried over anhydrous Na₂SO₄, filtered, and concentrated to dryness in vacuum to give a nearly yellow viscous oil. The oil was then subjected to silica gel chromatography (0-30% PE/EA) to afford methyl 5-{2-[2-(1,3-dioxolan-2-yl)-3-[(4-methoxyphenyl)methoxy]phenyl]ethynyl}-2-methylpyrazole-3-carboxylate (980 mg, 71.7%) as a yellow solid. MS (ESI), calcd. for C₂₅H₂₄N₂O₆, 448.16 m/z, found 449.20 [M+H]⁺.

### Step 3: Synthesis of 5-{2-[2-(1,3-dioxolan-2-yl)-3-[(4-methoxyphenyl)methoxy]phenyl]ethynyl}-2-methylpyrazole-3-carboxylic acid

Into a 40 mL vessel were added methyl 5-{2-[2-(1,3-dioxolan-2-yl)-3-[(4-methoxyphenyl) methoxy]phenyl]ethynyl}-2-methylpyrazole-3-carboxylate (500 mg, 1.115 mmol, 1 equiv), LiOH (1.11 mL, 3.345 mmol, 3 equiv, 3 mol/L), THF (3 mL) and MeOH (1 mL) and the mixture was stirred for 2 h at room temperature. The resulting mixture was concentrated and the residue was diluted with H₂O (5 mL). The resulting mixture was acidified to pH 6 with HCl (2 mol/L). The precipitated solid was collected by filtration and dried under in vacuum to afford 5-{2-[2-(1,3-dioxolan-2-yl)-3-[(4-methoxyphenyl)methoxy]phenyl]ethynyl}-2-methylpyrazole-3-carboxylic acid (480 mg, 99.10%) as a yellow solid. MS (ESI), calcd. for C₂₄H₂₂N₂O₆, 434.15 m/z, found 435.15[M+H]⁺.

### Step 4: Synthesis of (1R,3S)-3-[5-(5-{2-[2-(1,3-dioxolan-2-yl)-3-[(4-methoxyphenyl)methoxy]phenyl]ethynyl}-2-methylpyrazole-3-amido)-2H-pyrazol-3-yl]cyclopentyl N-isopropylcarbamate

Into a 40 mL vessel were added 5-{2-[2-(1,3-dioxolan-2-yl)-3-[(4-methoxyphenyl)methoxy]phenyl]ethynyl}-2-methylpyrazole-3-carboxylic acid (172.2 mg, 0.396 mmol, 1 equiv), pyridine (1 mL), (1R,3S)-3-(5-amino-2H-pyrazol-3-yl)cyclopentyl N-isopropylcarbamate (100 mg, 0.396 mmol, 1.00 equiv) and HBTU (180.4 mg, 0.475 mmol, 1.2 equiv) and the resulting mixture was stirred at 110° C. for 2 h. the reaction mixture was then treated with H₂O (10 mL) and extracted with EA (10 mL×3). The combined organic extracts were washed with brine (10 mL), dried over anhydrous Na₂SO₄, filtered, and concentrated to dryness in vacuum to give a nearly yellow viscous oil. The oil was purified by TLC to afford (1R,3S)-3-[5-(5-{2-[2-(1,3-dioxolan-2-yl)-3-[(4-methoxyphenyl)methoxy]phenyl]ethynyl}-2-methylpyrazole-3-amido)-2H-pyrazol-3-yl]cyclopentyl N-isopropylcarbamate (140 mg, 52.8%) as a yellow solid. MS (ESI), calcd. for C₃₆H₄₀N₆O₇, 668.30 m/z, found 669.35[M+H]⁺.

### Step 5: Synthesis of (1R,3S)-3-(5-{5-[2-(2-formyl-3-hydroxyphenyl)ethynyl]-2-methylpyrazole-3-amido}-2H-pyrazol-3-yl)cyclopentyl N-isopropylcarbamate

Into a 50 mL vessel were added (1R,3S)-3-[5-(5-{2-[2-(1,3-dioxolan-2-yl)-3-[(4-methoxyphenyl)methoxy]phenyl]ethynyl}-2-methylpyrazole-3-amido)-2H-pyrazol-3-yl]cyclopentyl N-isopropylcarbamate (100 mg, 0.150 mmol, 1 equiv) and HCl/1,4-dioxane (2 m L, 4 mol/L) and the resulting mixture was stirred at room temperature for 1 h. The reaction mixture was concentrated and the residue was purified by Prep-HPLC with the following conditions (Column: XSelect CSH C18 Column, 30×150 mm, 5 μm; Mobile Phase A: Water (0.1% FA), Mobile Phase B: ACN; Flow rate: 60 mL/min; Gradient: 34% B to 64% B in 7 min; Wave Length: 254 nm nm; RT1 (min): 6.66) to afford (1R,3S)-3-(5-{5-[2-(2-formyl-3-hydroxyphenyl)ethynyl]-2-methylpyrazole-3-amido}-2H-pyrazol-3-yl)cyclopentyl N-isopropylcarbamate (33.3 mg, 44.0%) as a yellow solid, MS (ESI), calcd. for C₂₆H₂₈N₆O₅, 504.21 m/z, found 505.15 [M+H]⁺. ¹H NMR (300 MHz, DMSO-d₆) δ (ppm): δ 12.28 (s, 1H), 11.23 (s, 1H), 10.87 (s, 1H), 10.27 (s, 1H), 7.76-7.83 (m, 1H), 7.70 (dd, J=8.6, 2.3 Hz, 1H), 7.31-7.37 (m, 1H), 7.04-7.13 (m, 1H), 6.84-7.00 (m, 1H), 6.41-6.47 (m, 1H), 4.99-5.05 (m, 1H), 4.09-4.14 (m, 3H), 3.49-3.68 (m, 1H), 3.09 (t, J=8.8 Hz, 1H), 1.99-2.11 (m, 1H), 1.81-1.85 (m, 1H), 1.71-1.79 (m, 1H), 1.56-1.66 (m, 2H), 1.45-1.51 (m, 1H), 1.04 (d, J=6.5 Hz, 6H).

### Example 6.12: Synthesis of Compound 30: (1R,3S)-3-(5-{5-[2-(2-formyl-3-hydroxyphenyl)ethyl]-2-methylpyrazole-3-amido}-2H-pyrazol-3-yl)cyclopentyl N-isopropylcarbamate

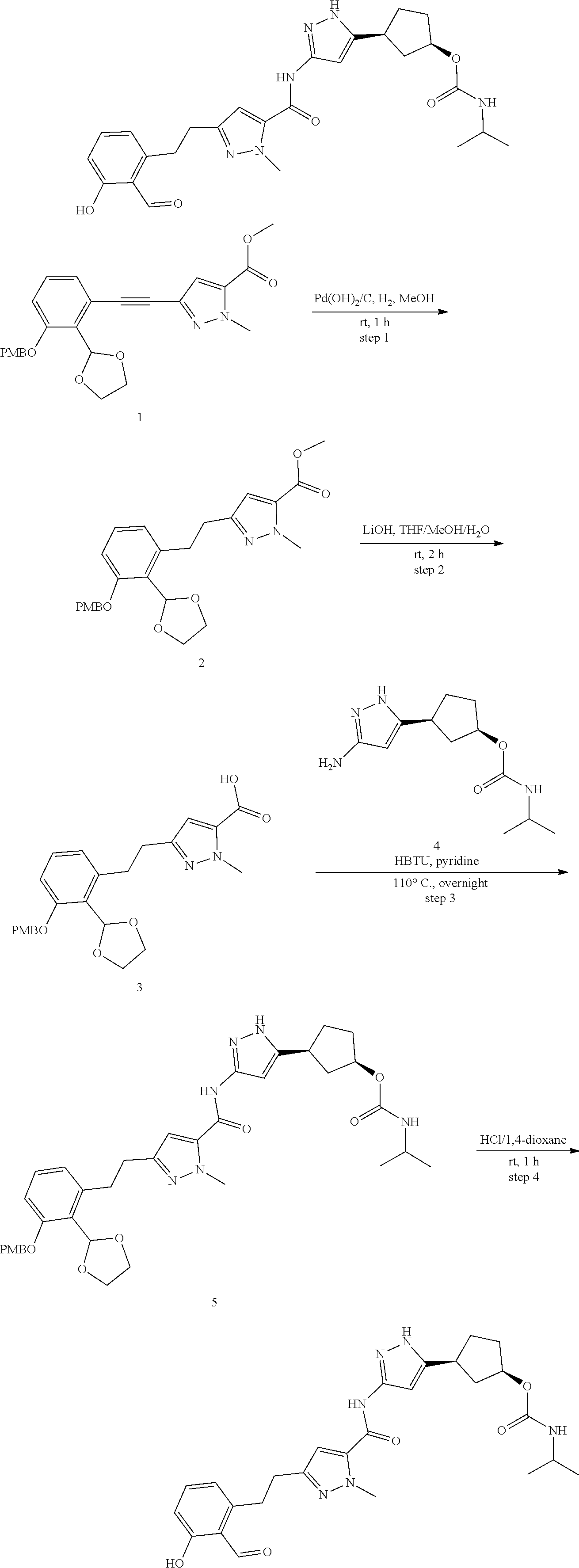

### Step 1: Synthesis of methyl 5-{2-[2-(1,3-dioxolan-2-yl)-3-[(4-methoxyphenyl)methoxy]phenyl]ethyl}-2-methylpyrazole-3-carboxylate

methyl 3-((2-(1,3-dioxolan-2-yl)-3-((4-methoxybenzyl)oxy)phenyl)ethynyl)-1-methyl-1H-pyrazole-5-carboxylate (400 mg, 0.921 mmol, 1 equiv), a stir bar, were added to a 40 mL vial suspended methanol (1 mL) and stirred until homogeneous. Pd(OH)₂/C (65 mg) was added and the mixture was stirred at room temperature for 1 h under an atmosphere of H₂. The reaction mixture was filtered over Celite and evaporated under reduced pressure to afford 5-{2-[2-(1,3-dioxolan-2-yl)-3-[(4-methoxyphenyl)methoxy]phenyl]ethyl}-2-methylpyrazole-3-carboxylate (120 mg, 29.72%) as a colorless oil. MS (ESI), calcd. for C₂₅H₂₈N₂O₆, 452.19 m/z, found 453.25 [M+H]⁺.

### Step 2: Synthesis of 5-{2-[2-(1,3-dioxolan-2-yl)-3-[(4-methoxyphenyl)methoxy]phenyl]ethyl}-2-methylpyrazole-3-carboxylic acid

Into a 40 mL vial were added methyl 5-{2-[2-(1,3-dioxolan-2-yl)-3-[(4-methoxyphenyl)methoxy]phenyl]ethyl}-2-methylpyrazole-3-carboxylate (120 mg, 0.265 mmol, 1 equiv), lithium hydroxide 3 M (0.27 mL, 0.795 mmol, 3 equiv) and MeOH (1 mL) and the resulting mixture was stirred at room temperature for 2 h. The resulting mixture was concentrated and diluted with H₂O (2 mL). The mixture was acidified to pH 6 with HCl (2 mol/L). The precipitated solid was collected by filtration and dried under in vacuum to afford 5-{2-[2-(1,3-dioxolan-2-yl)-3-[(4-methoxyphenyl)methoxy]phenyl]ethyl}-2-methylpyrazole-3-carboxylic acid (50 mg, 43.0%) as a yellow solid. MS (ESI), calcd. for C₂₄H₂₆N₂O₆, 438.18 m/z, found 439.15 [M+H]⁺.

### Step 3: Synthesis of (1R,3S)-3-[5-(5-{2-[2-(1,3-dioxolan-2-yl)-3-[(4-methoxyphenyl)methoxy]phenyl]ethyl}-2-methylpyrazole-3-amido)-2H-pyrazol-3-yl]cyclopentyl N-isopropyl carbamate

Into a 25 mL vessel were added 5-{2-[2-(1,3-dioxolan-2-yl)-3-[(4-methoxyphenyl)methoxy]phenyl]ethyl}-2-methylpyrazole-3-carboxylic acid (55 mg, 0.125 mmol, 1 equiv), pyridine (5 mL), (1R,3S)-3-(5-amino-2H-pyrazol-3-yl)cyclopentyl N-isopropylcarbamate (31.7 mg, 0.125 mmol, 1 equiv) and HBTU (57.1 mg, 0.150 mmol, 1.2 equiv) and the resulting mixture was stirred at 110° C. overnight. The reaction mixture was then treated with H₂O (5 mL), extracted with EA (5 mL×3). The combined organic layers were washed with brine (5 mL), dried over anhydrous Na₂SO₄, filtered, and concentrated to dryness in vacuum to give a nearly yellow viscous oil. The oil was purified by TLC to afford (1R,3S)-3-[5-(5-{2-[2-(1,3-dioxolan-2-yl)-3-[(4-methoxyphenyl) methoxy]phenyl]ethyl}-2-methylpyrazole-3-amido)-2H-pyrazol-3-yl]cyclopentyl N-isopropyl-carbamate (25 mg, 29.6%) as a yellow solid. MS (ESI), calcd. for C₃₆H₄₄N₆O₇, 672.33 m/z, found 673.25 [M+H]⁺.

### Step 4: Synthesis of (1R,3S)-3-(5-{5-[2-(2-formyl-3-hydroxyphenyl)ethyl]-2-methylpyrazole-3-amido}-2H-pyrazol-3-yl)cyclopentyl N-isopropylcarbamate

Into a 50 mL vessel were added (1R,3S)-3-[5-(5-{2-[2-(1,3-dioxolan-2-yl)-3-[(4-methoxyphenyl) methoxy]phenyl]ethyl}-2-methylpyrazole-3-amido)-2H-pyrazol-3-yl]cyclopentyl N-isopropyl-carbamate (20 mg, 0.030 mmol, 1 equiv), HCl/1,4-dioxane (1 mL, 2 mol/L) and the resulting mixture was stirred at room temperature for 1 h. The reaction mixture was concentrated and the residue was purified by Prep-HPLC with the following conditions (Column: Xselect CSH C18 OBD Column, 30×150 mm, 5 μm; Mobile Phase A: Water (0.05% TFA), Mobile Phase B: ACN; Flow rate: 60 mL/min; Gradient: 29% B to 59% B in 7 min; Wave Length: 254/220 nm; RT1 (min): 5.82) to afford (1R,3S)-3-(5-{5-[2-(2-formyl-3-hydroxyphenyl)ethyl]-2-methylpyrazole-3-amido}-2H-pyrazol-3-yl)cyclopentyl N-isopropylcarbamate (1.9 mg, 12.43%) as an off-white solid, MS (ESI), calcd. for C₂₆H₃₂N₆O₅, 508.24 m/z, found 509.30 [M+H]⁺. ¹H NMR (300 MHz, DMSO-d₆) δ (ppm): δ 10.65 (s, 1H), 10.54 (s, 1H), 10.23 (s, 1H), 7.53 (d, J=2.4 Hz, 1H), 7.41 (dd, J=8.5, 2.4 Hz, 1H), 6.88-7.01 (m, 3H), 6.37-6.43 (m, 1H), 4.98-5.04 (m, 1H), 4.05 (s, 3H), 3.49-3.65 (m, 1H), 3.05-3.11 (m, 1H), 2.75-2.89 (m, 4H), 2.61-2.56 (m, 1H), 1.99-2.08 (m, 1H), 1.87-1.93 (m, 1H), 1.70-1.79 (m, 2H), 1.59-1.65 (m, 1H), 1.04 (d, J=6.5 Hz, 6H).

### Example 6.13: Synthesis of Compound 45: (1R,3S)-3-(3-(3-(6-fluoro-2-formyl-3-hydroxyphenyl)-1-methyl-1H-pyrazole-5-carboxamido)-1H-pyrazol-5-yl)cyclopentyl isopropylcarbamate

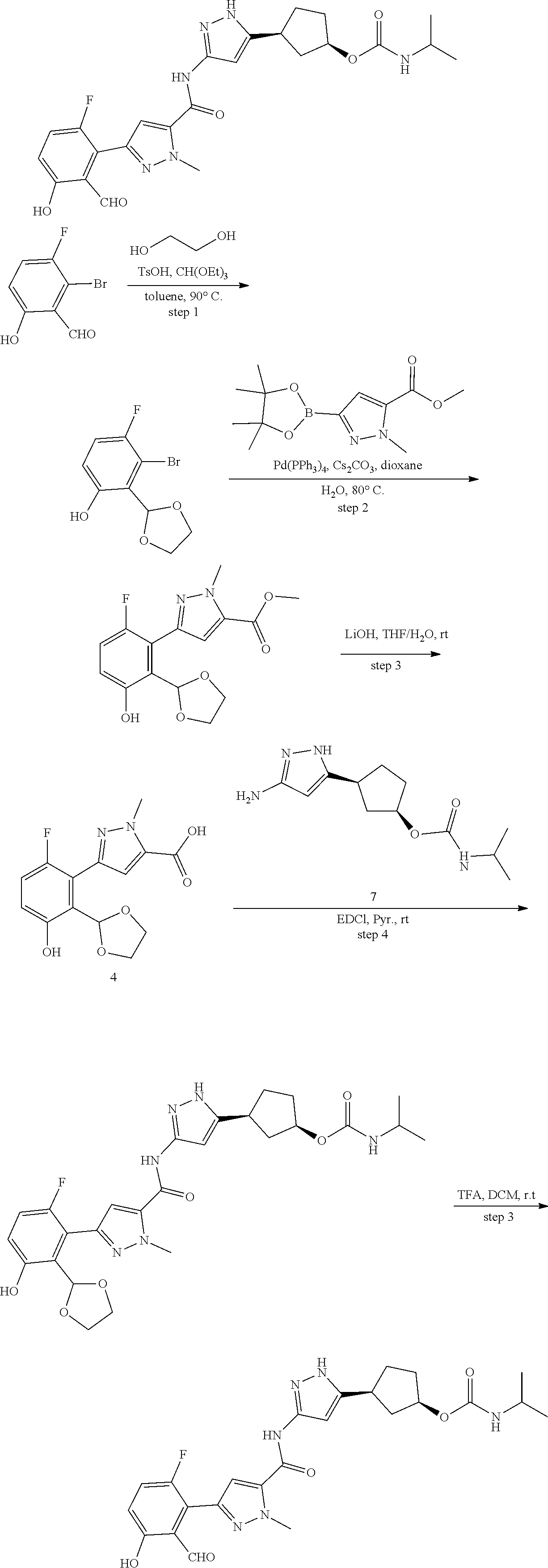

### Step 1: Synthesis of 3-bromo-2-(1,3-dioxolan-2-yl)-4-fluorophenol

To a stirred solution of 2-bromo-4-fluoro-6-hydroxybenzaldehyde (1.5 g, 6.849 mmol, 1 equiv) and TsOH (0.18 g, 1.027 mmol, 0.15 equiv) in toluene (15 mL) was added ethylene glycol (2.13 g, 34.245 mmol, 5 equiv) and triethyl orthoformate (3.04 g, 20.547 mmol, 3 equiv) dropwise at 25° C. The resulting mixture was stirred for additional 3 h at 120° C., then cooled to room temperature and diluted with water. The resulting mixture was extracted with EA (50×3 mL). The combined organic layers were washed with brine (50×3 mL), dried over anhydrous Na₂SO₄. The residue was purified by silica gel column chromatography, eluted with PE/EA (0-100%) to afford 3-bromo-2-(1,3-dioxolan-2-yl)-5-fluorophenol (900 mg, 49.95%) as a yellow oil. MS (ESI) calcd. for C₉H₈BrFO₃, 261.96 m/z, found 263.20 [M+H]⁺.

### Step 2: Synthesis of methyl 3-(2-(1,3-dioxolan-2-yl)-6-fluoro-3-hydroxyphenyl)-1-methyl-1H-pyrazole-5-carboxylate

To a stirred solution of 3-bromo-2-(1,3-dioxolan-2-yl)-4-fluorophenol (800.0 mg, 3.041 mmol, 1 equiv) in 1,4-dioxane (20 mL) and H₂O (4 mL) were added methyl 2-methyl-5-(4,4,5,5-tetramethyl-1,3,2-dioxaborolan-2-yl)pyrazole-3-carboxylate (971.09 mg, 3.649 mmol, 1.2 equiv), Cs₂CO₃ (2477.13 mg, 7.603 mmol, 2.5 equiv) and Pd(PPh₃)₄ (527.14 mg, 0.456 mmol, 0.15 equiv) at 25° C. The resulting mixture was stirred for 3 h at 80° C. under N₂ atmosphere. The reaction was monitored by LCMS. The resulting mixture was extracted with EA (30×3 mL). The combined organic layers were washed with saturated NaCl solution (30×3 mL), dried over anhydrous Na₂SO₄. After filtration, the filtrate was concentrated under reduced pressure. The residue was purified by silica gel column chromatography, eluted with PE:EA (0-100%) to afford methyl 3-(2-(1,3-dioxolan-2-yl)-6-fluoro-3-hydroxyphenyl)-1-methyl-1H-pyrazole-5-carboxylate (350.0 mg, 28.57%) as a yellow oil. MS (ESI) calcd. for C₁₅H₁₅FN₂O₅, 322.02 m/z, found 333.20 [M+H]⁺.

### Step 3: Synthesis of 5-[2-(1,3-dioxolan-2-yl)-6-fluoro-3-hydroxyphenyl]-2-methylpyrazole-3-carboxylic acid

A solution of methyl 5-[2-(1,3-dioxolan-2-yl)-6-fluoro-3-hydroxyphenyl]-2-methylpyrazole-3-carboxylate (300.0 mg, 0.931 mmol, 1 equiv) and LiOH (55.73 mg, 2.328 mmol, 2.5 equiv) in H₂O (2 mL) and THF (2 mL) was stirred for 2 h at 25° C. The resulting mixture was stirred for 1 h at room temperature. The reaction was monitored by LCMS. The mixture acidified to pH ˜5 with HCl (2 M). The precipitated solids were collected by filtration. This resulted in afford 5-[2-(1,3-dioxolan-2-yl)-6-fluoro-3-hydroxyphenyl]-2-methylpyrazole-3-carboxylic acid (150.0 mg, 52.28%) as a yellow solid. MS (ESI) calcd. for C₁₄H₁₃FN₂O₅, 308.26 m/z, found 309.10 [M+H]⁺

### Step 4: Synthesis of (1R,3S)-3-(5-{5-[2-(1,3-dioxolan-2-yl)-6-fluoro-3-hydroxyphenyl]-2-methylpyrazole-3-amido}-2H-pyrazol-3-yl)cyclopentyl N-isopropylcarbamate

A solution of 5-[2-(1,3-dioxolan-2-yl)-6-fluoro-3-hydroxyphenyl]-2-methylpyrazole-3-carboxylic acid (130.0 mg, 0.422 mmol, 1 equiv) and (1R,3S)-3-(5-amino-2H-pyrazol-3-yl)cyclopentyl N-isopropylcarbamate (106.41 mg, 0.422 mmol, 1 equiv) and EDCI (121.26 mg, 0.633 mmol, 1.5 equiv) in pyridine (5 mL) was stirred for 5 h at rt. The resulting mixture was diluted with water and extracted with EA (5×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE:EA (0-100%) (1R,3S)-3-(5-{5-[2-(1,3-dioxolan-2-yl)-6-fluoro-3-hydroxyphenyl]-2-methylpyrazole-3-amido}-2H-pyrazol-3-yl)cyclopentyl N-isopropyl carbamate (70.0 mg, 30.59%) as a yellow oil. MS (ESI) calcd. for C₁₄H₁₃FN₂O₅, 542.26 m/z, found 543.25 [M+H]⁺

### Step 5: Synthesis of (1R,3S)-3-{5-[5-(6-fluoro-2-formyl-3-hydroxyphenyl)-2-methylpyrazole-3-amido]-2H-pyrazol-3-yl}cyclopentyl N-isopropylcarbamate

A solution of (1R,3S)-3-(5-{5-[2-(1,3-dioxolan-2-yl)-6-fluoro-3-hydroxyphenyl]-2-methyl pyrazole-3-amido}-2H-pyrazol-3-yl)cyclopentyl N-isopropylcarbamate (70.0 mg, 0.129 mmol, 1 equiv) in DCM (2 mL) and TFA (0.5 mL) was stirred for 1 h at r.t The reaction was monitored by LCMS. The resulting mixture was concentrated under reduced pressure. The residue was purified by reversed-phase flash chromatography with the following conditions: column, C18 silica gel; mobile phase, ACN/H₂O (0.05% TFA Modifier), 10% to 50% gradient in 30 min; detector, UV 254 nm to afford (1R,3S)-3-{5-[5-(6-fluoro-2-formyl-3-hydroxyphenyl)-2-methylpyrazole-3-amido]-2H-pyrazol-3-yl}cyclopentyl N-isopropylcarbamate (5.0 mg, 7.70%) as a light green solid. MS (ESI) calcd. for C₂₄H₂₇FN₆O₅, 498.20 m/z, found 499.30 [M+H]⁺. ¹H NMR (400 MHz, DMSO-d6) δ 11.44 (br s, 1H), 10.99 (s, 1H), 10.06 (s, 1H), 7.58-7.63 (m, 1H), 7.52-7.53 (m, 1H), 7.06-7.10 (m, 1H), 6.97-6.98 (m, 1H), 6.45 (s, 1H), 4.96-5.01 (m, 1H), 4.18 (s, 3H), 3.56-3.61 (m, 1H), 3.07-3.11 (m, 1H), 2.45-2.49 (m, 1H), 2.02-2.08 (m, 1H), 1.91-1.92 (m, 1H), 1.76-1.87 (m, 3H), 1.04 (d, J=6.8 Hz 6H). ¹⁹F NMR (376 MHz, DMSO-d₆) δ (ppm): −74.90, −125.44.

### Example 6.14: Synthesis of Compound 54: (1R,3S)-3-(3-(3-(difluoromethyl)-1-(2-formyl-3-hydroxyphenyl)-1H-pyrazole-4-carboxamido)-1H-pyrazol-5-yl)cyclopentyl isopropylcarbamate

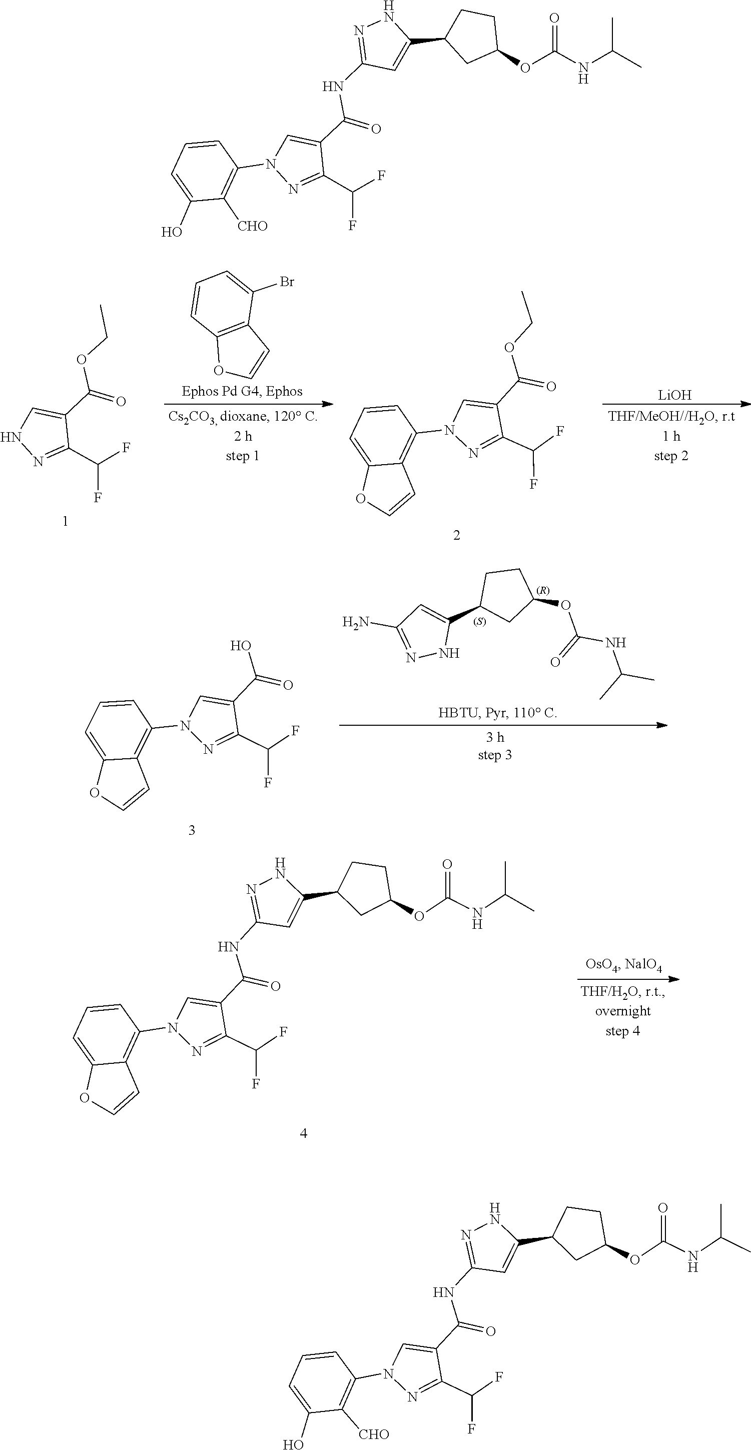

### Step 1: Synthesis of ethyl 1-(1-benzofuran-4-yl)-3-(difluoromethyl)pyrazole-4-carboxylate

A solution of ethyl 3-(difluoromethyl)-1H-pyrazole-4-carboxylate (300 mg, 1.578 mmol, 1 equiv), Ephos Pd G4 (156.4 mg, 0.170 mmol, 0.11 equiv), 4-bromo-1-benzofuran (332.3 mg, 1.687 mmol, 1.07 equiv), Ephos (182 mg, 0.340 mmol, 0.22 equiv), and Cs₂CO₃ (1.66 g, 12.490 mmol. 7.92 equiv), in 1,4-dioxane (4 mL) was stirred for 2 h at 120° C. under N₂ atmosphere. After cooling down to rt., the solution was quenched with water and extracted with EA three times. The combined organic layers were washed with brine, dried over anhydrous sodium sulfate and concentrated under vacuum to afford yellow solid. The solid was then subjected to silica gel chromatography (0-40% EtOAc/petroleum ether) to afford ethyl 1-(1-benzofuran-4-yl)-3-(difluoromethyl)pyrazole-4-carboxylate (160 mg, 33.1%) as a yellow oil. MS (ESI) calcd. for C₁₅H₁₂F₂N₂O₃, 306.08 m/z, found 307.05 [M+H]+

### Step 2: Synthesis of 1-(1-benzofuran-4-yl)-3-(difluoromethyl)pyrazole-4-carboxylic acid

A solution of ethyl 1-(1-benzofuran-4-yl)-3-(difluoromethyl)pyrazole-4-carboxylate (150 mg, 0.490 mmol, 1 equiv) in MeOH (0.5 mL) and THF (0.15 mL) was treated with LiOH (0.4 mL, 3 mol/L) and the mixture was stirred for 1 h at room temperature. The resulting mixture was concentrated and diluted with H₂O (2 mL). The mixture was acidified to pH 6 with HCl (2 mol/L). The precipitated solid was collected by filtration and dried under vacuum to afford 1-(1-benzofuran-4-yl)-3-(difluoromethyl)pyrazole-4-carboxylic acid (130 mg, 95.4%) as a yellow oil. MS (ESI) calcd. for C₁₃H₈F₂N₂O₃, 278.05 m/z, found 279.05 [M+H]⁺.

### Step 3: Synthesis of (1R,3S)-3-{5-[1-(1-benzofuran-4-yl)-3-(difluoromethyl)pyrazole-4-amido]-2H-pyrazol-3-yl}cyclopentyl N-isopropylcarbamate

A solution of 1-(1-benzofuran-4-yl)-3-(difluoromethyl)pyrazole-4-carboxylic acid (100 mg, 0.359 mmol, 1 equiv) in pyridine (1 mL, 0.008 mmol, 0.04 equiv) was treated with (1R,3S)-3-(5-amino-2H-pyrazol-3-yl)cyclopentyl N-isopropylcarbamate (164.15 mg, 0.651 mmol, 1.81 equiv) and EDCI (186.93 mg, 1.203 mmol) and the resulting mixture was stirred for 3 h at room temperature. After the reaction was finished, the reaction mixture was quenched with H₂O (5 mL) and the mixture was extracted with EtOAc (3×5 mL). The organic layers were combined, dried over Na₂SO₄, filtered and concentrated. The residue obtained was purified by silica gel chromatography (0-20% ethyl acetate/petroleum ether) to afford the (1R,3S)-3-{5-[1-(1-benzofuran-4-yl)-3-(difluoromethyl)pyrazole-4-amido]-2H-pyrazol-3-yl}cyclopentyl N-isopropylcarbamate (80 mg, 43.4%) as an orange oil. MS (ESI) calcd. for C₂₅H₂₆F₂N₆O₄, 512.20 m/z, found 513.80 [M+H]⁺.

### Step 4: Synthesis of (1R,3S)-3-{5-[3-(difluoromethyl)-1-(2-formyl-3-hydroxyphenyl)pyrazole-4-amido]-2H-pyrazol-3-yl}cyclopentyl N-isopropylcarbamate

To a solution of (1R,3S)-3-{5-[1-(1-benzofuran-4-yl)-3-(difluoromethyl)pyrazole-4-amido]-2H-pyrazol-3-yl}cyclopentyl N-isopropylcarbamate (60 mg, 0.117 mmol, 1 equiv) in THF (0.5 mL) and H₂O (0.5 mL) was added osmium tetroxide (0.6 mL, 2.5% w/w) dropwise at 0° C. After stirred for 30 min, NaIO₄ (120 mg, 0.625 mmol, 5.34 equiv) was added into the mixture, and then the reaction mixture was stirred at room temperature overnight. After the reaction was finished, the reaction mixture was quenched with H₂O (5 mL), and the mixture was extracted with EtOAc (3×5 mL). The organic layers were combined, dried over Na₂SO₄, filtered and concentrated. Then the crude product was purified by Prep-HPLC (Column: Xselect CSH Prep C18 OBD Column, 30×150 mm, 5 um; Mobile Phase A: Water (0.05% TFA), Mobile Phase B: ACN; Flow rate: 60 mL/min; Gradient: 33% B to 55% B in 10 min; Wave Length: 254/220 nm) to afford (1R,3S)-3-{5-[3-(difluoromethyl)-1-(2-formyl-3-hydroxyphenyl)pyrazole-4-amido]-2H-pyrazol-3-yl}cyclopentyl N-isopropylcarbamate (1.8 mg, 2.9%) as a white solid. MS (ESI) calcd. for C₂₄H₂₆F₂N₆O₅, 516.19 m/z, found 517.25 [M+H]⁺. 1H NMR (300 MHz, DMSO-d₆) δ 11.51 (s, 1H), 10.73 (s, 1H), 10.00 (s, 1H), 9.02 (s, 1H), 7.54-7.82 (m, 1H), 7.42 (s, 1H), 7.24-7.35 (m, 2H), 6.85-7.17 (m, 1H), 6.45 (s, 1H), 4.82-5.21 (m, 1H), 3.50-3.66 (m, 3H), 3.08-3.21 (m, 1H), 2.03-2.11 (m, 1H), 1.89-1.99 (m, 1H), 1.76-1.82 (m, 2H), 1.54-1.73 (m, 1H), 0.92-1.04 (m, 6H).

### Example 6.15: Synthesis of Compound 79: (1R,3S)-3-(5-{2-[1-(2-formyl-3-hydroxy-5-methoxyphenyl) pyrazol-4-yl]acetamido}-2H-pyrazol-3-yl) cyclopentyl N-isopropylcarbamate

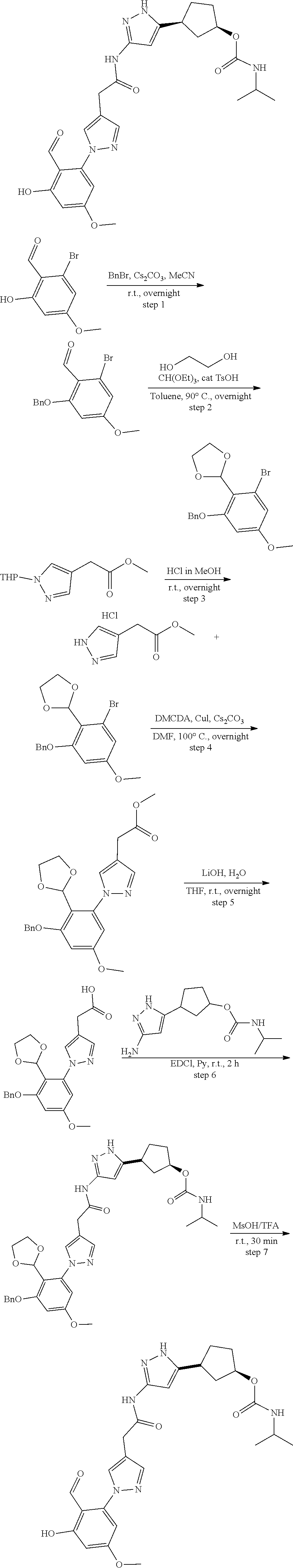

### Step 1: Synthesis of 2-(benzyloxy)-6-bromo-4-methoxybenzaldehyde

2-bromo-6-hydroxy-4-methoxybenzaldehyde (3 g, 12.984 mmol), Cs₂CO₃ (8.46 g, 25.968 mmol), a stir bar and CH₃CN (60 mL) were added to an oven-dried 250 mL round bottom flask and stirred until homogenous, then treated with BnBr (3.33 g, 19.476 mmol). The resulting mixture was stirred overnight at rt, then quenched with saturated NH₄Cl (20 mL) solution at 0° C. The resulting mixture was extracted with EA (100 mL) for three times. The combined organic layers were washed with brine (10 mL), dried over anhydrous sodium sulfate, filtered, and concentrated under vacuum. The residue was purified by silica gel chromatography (0-25% EtOAc/PE) to afford 2-(benzyloxy)-6-bromo-4-methoxybenzaldehyde (3.5 g, 93.52%) as a white solid. MS (ESI) calcd. for C₁₅H₁₃BrO₃, 320.00, found, 320.90 [M+H]⁺.

### Step 2: Synthesis of 2-[2-(benzyloxy)-6-bromo-4-methoxyphenyl]-1,3-dioxolane

2-(benzyloxy)-6-bromo-4-methoxybenzaldehyde (3.5 g, 10.898 mmol), triethyl orthoformate (4.85 g, 32.694 mmol), ethylene glycol (3.38 g, 54.490 mmol), TsOH (0.38 g, 2.180 mmol), a stir bar and toluene (35 mL) were added to an oven-dried 250 mL round bottom flask and stirred until homogenous. The resulting mixture was stirred overnight at 90° C. The resulting mixture was cooled to rt. And concentrated under vacuum. The residue was purified by silica gel chromatography (0-83% DCM/PE) to afford 2-[2-(benzyloxy)-6-bromo-4-methoxyphenyl]-1,3-dioxolane (3.2 g, 80.40%) as a white solid. MS (ESI) calcd. for C₁₇H₁₇BrO₄, 364.03 m/z, found, 364.95 [M+H]⁺.

### Step 3: Synthesis of methyl 2-(1H-pyrazol-4-yl)acetate

Methyl 2-[1-(oxan-2-yl) pyrazol-4-yl]acetate (450 mg, 2.007 mmol), a stir bar and HCl (15 mL, 4 mol/L in MeOH) were added to an oven-dried 100 mL round bottom flask and stirred until homogenous. The resulting mixture was stirred overnight at rt. The resulting mixture was concentrated under vacuum to afford methyl 2-(1H-pyrazol-4-yl)acetate hydrochloride (250 mg, 75.5%) as a yellow solid. MS (ESI) calcd. for C₆H₈N₂O₂, 140.06, found, 141.00 [M+H]⁺.

### Step 4: Synthesis of methyl 2-{1-[3-(benzyloxy)-2-(1,3-dioxolan-2-yl)-5-methoxyphenyl]pyrazol-4-yl}acetate

Methyl 2-(1H-pyrazol-4-yl)acetate (230 mg, 1.641 mmol), 2-[2-(benzyloxy)-6-bromo-4-methoxyphenyl]-1,3-dioxolane (660 mg, 1.805 mmol), N1, N2-dimethylcyclohexane-1,2-diamine (70 mg, 0.492 mmol), Cs₂CO₃ (1337 mg, 4.103 mmol), CuI (62.5 mg, 0.328 mmol), a stir bar and DMF (10 mL) were added to a nitrogen-purged 100 mL round-bottom flask and stirred until homogenous. The resulting mixture was stirred overnight at 100° C. under nitrogen atmosphere, then cooled to rt. The resulting mixture was filtered and washed with CH₃CN. The filtrate was concentrated under vacuum. The residue was purified by reverse-phase chromatography (0-60% ACN/H₂O with 10 mM NH₄HCO₃) to afford methyl 2-{1-[3-(benzyloxy)-2-(1,3-dioxolan-2-yl)-5-methoxyphenyl]pyrazol-4-yl}acetate (240 mg, 34.45%) as a yellow solid. MS (ESI) calcd. for C₂₃H₂₄N₂O₆, 424.16 m/z, found, 425.15 m/z [M+H]⁺.

### Step 5: Synthesis of {1-[3-(benzyloxy)-2-(1,3-dioxolan-2-yl)-5-methoxyphenyl]pyrazol-4-yl}acetic acid

Methyl 2-{1-[3-(benzyloxy)-2-(1,3-dioxolan-2-yl)-5-methoxyphenyl]pyrazol-4-yl}acetate (230 mg, 0.542 mmol), LiOH (38.9 mg, 1.626 mmol), MeOH (2 mL), H₂O (2 mL), a stir bar and THF (6 mL) were added to an oven-dried 100 mL round-bottom flask and stirred until homogenous. The resulting mixture was stirred overnight at rt, then concentrated under vacuum. The pH of the mixture was adjusted to around 4 with 10 wt % HOAc solution. The resulting mixture was extracted with EA (50 mL) three times. The combined organic layers were washed with brine (10 mL), dried over anhydrous sodium sulfate, filtered and concentrated under vacuum to afford {1-[3-(benzyloxy)-2-(1,3-dioxolan-2-yl)-5-methoxyphenyl]pyrazol-4-yl}acetic acid (190 mg, 85.43%) as a yellow solid. MS (ESI) calcd. for C₂₂H₂₂N₂O₆, 410.15, found, 411.15 [M+H]⁺.

### Step 6: Synthesis of (1R,3S)-3-[5-(2-{1-[3-(benzyloxy)-2-(1,3-dioxolan-2-yl)-5-methoxyphenyl]pyrazol-4-yl}acetamido)-2H-pyrazol-3-yl]cyclopentyl N-isopropylcarbamate

{1-[3-(benzyloxy)-2-(1,3-dioxolan-2-yl)-5-methoxyphenyl]pyrazol-4-yl}acetic acid (170 mg, 0.415 mmol), (1R,3S)-3-(5-amino-2H-pyrazol-3-yl)cyclopentyl N-isopropylcarbamate (95 mg, 0.377 mmol), a stir bar and pyridine (3 mL) were added to an oven-dried 50 mL round-bottom flask and stirred until homogenous, then treated with EDCI (144.3 mg, 0.754 mmol). The resulting mixture was stirred for 2 h at rt, then concentrated under vacuum. The residue was purified by reverse-phase chromatography (0-51% ACN/H₂O with 10 mM NH₄HCO₃) to afford (1R,3S)-3-[5-(2-{1-[3-(benzyloxy)-2-(1,3-dioxolan-2-yl)-5-methoxyphenyl]pyrazol-4-yl}acetamido)-2H-pyrazol-3-yl]cyclopentyl N-isopropylcarbamate (80 mg, 32.96%) as a yellow oil. MS (ESI) calcd. for C₃₄H₄₀N₆O₇, 644.30 m/z, found, 645.35 m/z [M+H]⁺.

### Step 7: Synthesis of (1R,3S)-3-(5-{2-[1-(2-formyl-3-hydroxy-5-methoxyphenyl)pyrazol-4-yl]acetamido}-2H-pyrazol-3-yl)cyclopentyl N-isopropylcarbamate

(1R,3S)-3-[5-(2-{1-[3-(benzyloxy)-2-(1,3-dioxolan-2-yl)-5-methoxyphenyl]pyrazol-4-yl}acetamido)-2H-pyrazol-3-yl]cyclopentyl N-isopropylcarbamate (80 mg, 0.124 mmol), methanesulfonic acid (1 mL), a stir bar and TFA (5 mL) were added to an oven-dried 50 mL round bottom flask and stirred until homogenous. The resulting mixture was stirred at rt for 30 min, then concentrated under vacuum. The residue was purified by Prep-HPLC with (Column: Xselect CSH Prep C18 OBD Column, 30×150 mm, 5 um; Mobile Phase A: Water (0.1% FA), Mobile Phase B: ACN; Flow rate: 60 mL/min; Gradient: 25% B to 47% B in 10 min; Wave Length: 254/220 nm; RT1 (min): 9.5) to afford (1R,3S)-3-(5-{2-[1-(2-formyl-3-hydroxy-5-methoxyphenyl)pyrazol-4-yl]acetamido}-2H-pyrazol-3-yl)cyclopentyl N-isopropylcarbamate (7.1 mg, 10.94%) as a white solid. MS (ESI) calcd. for C₂₅H₃₀N₆O₆, 510.22, found, 511.25 [M+H]⁺. ¹H NMR (400 MHz, DMSO-d₆) δ 12.19 (s, 1H), 10.49 (s, 1H), 9.77 (s, 1H), 8.22 (s, 1H), 7.76 (s, 1H), 6.94 (t, J=6.8 Hz, 1H), 6.69 (d, J=2.4 Hz, 1H), 6.57 (d, J=2.3 Hz, 1H), 6.31 (s, 1H), 4.99 (s, 1H), 3.87 (s, 4H), 3.56 (s, 3H), 3.09-2.98 (m, 1H), 2.49-2.41 (m, 1H), 2.05-1.93 (m, 1H), 1.93-1.80 (m, 1H), 1.76-1.63 (m, 2H), 1.62-1.51 (m, 1H), 1.06-0.97 (m, 6H).

### Example 6.16: Synthesis of Compound 89; (1R,3S)-3-{5-[trans-2-(2-formyl-3-hydroxy-5-methoxyphenyl)cyclopropaneamido]-2H-pyrazol-3-yl}cyclopentyl N-isopropylcarbamate and Compound 90; (1R,3S)-3-{5-[(1S,2S)-2-(2-formyl-3-hydroxy-5-methoxyphenyl)cyclopropaneamido]-2H-pyrazol-3-yl}cyclopentyl N-isopropylcarbamate

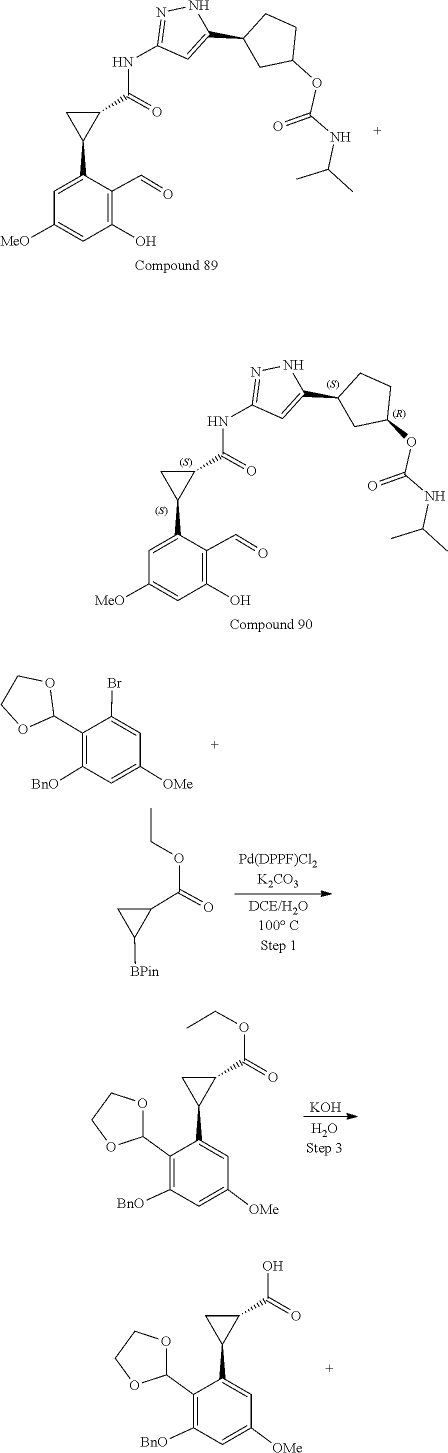

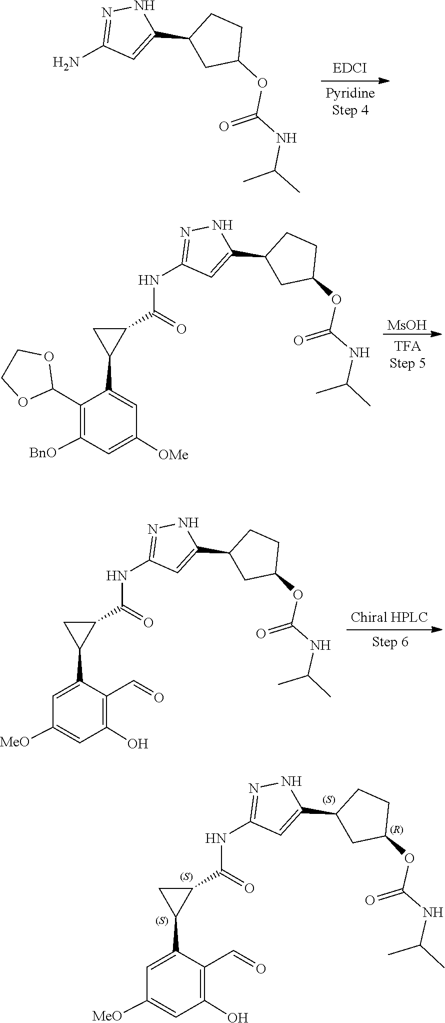

### Step 1: Synthesis of ethyl 2-[3-(benzyloxy)-2-(1,3-dioxolan-2-yl)-5-methoxyphenyl]cyclopropane-1-carboxylate

2-[2-(benzyloxy)-6-bromo-4-methoxyphenyl]-1,3-dioxolane (800 mg, 2.190 mmol), ethyl 2-(4,4,5,5-tetramethyl-1,3,2-dioxaborolan-2-yl)cyclopropane-1-carboxylate (578.5 mg, 2.409 mmol), Pd(dppf)Cl₂ (160.3 mg, 0.219 mmol), K₂CO₃ (1210.9 mg, 8.760 mmol), DCE (10 mL), a stir bar and H₂O (2 mL) were added to a nitrogen-purged 100 mL round-bottom flask and stirred until homogenous. The resulting mixture was stirred overnight at 100° C. under nitrogen atmosphere, then cooled to rt and quenched with water. The resulting mixture was extracted with DCM (50 mL) for three times. The combined organic layers were washed with brine (10 mL), dried over anhydrous sodium sulfate, filtered, and concentrated under vacuum. The residue was purified by silica gel chromatography (0-25% EtOAc/PE) to afford trans-ethyl 2-[3-(benzyloxy)-2-(1,3-dioxolan-2-yl)-5-methoxyphenyl]cyclopropane-1-carboxylate (740 mg, 84.79%) as a yellow oil. MS (ESI) calcd. for C₂₃H₂₆O₆, 398.17, found, 399.15[M+H]⁺.

### Step 2: Synthesis of 2-[3-(benzyloxy)-2-(1,3-dioxolan-2-yl)-5-methoxyphenyl]cyclopropane-1-carboxylic acid

Trans-ethyl 2-[3-(benzyloxy)-2-(1,3-dioxolan-2-yl)-5-methoxyphenyl]cyclopropane-1-carboxy-late (400 mg, 1.004 mmol), KOH (2.5 mL, 2 mol/L), a stir bar and EtOH (8 mL) were added to an oven-dried 50 mL round-bottom flask and stirred until homogenous. The resulting mixture was stirred overnight at rt, then concentrated under vacuum. The pH of the mixture was adjusted to around pH 4 with 10 wt % HOAc solution. The resulting mixture was extracted with EA (50 mL) for three times. The combined organic layers were washed with brine (10 mL), dried over anhydrous sodium sulfate, filtered and concentrated under vacuum to afford trans-2-[3-(benzyloxy)-2-(1,3-dioxolan-2-yl)-5-methoxyphenyl]cyclopropane-1-carboxylic acid (350 mg, 94.13%) as a yellow oil. MS (ESI) calcd. for C₂₁H₂₂O₆, 370.14 m/z, found, 371.05 m/z[M+H]⁺.

### Step 3: Synthesis of (1R,3S)-3-(5-{trans-2-[3-(benzyloxy)-2-(1,3-dioxolan-2-yl)-5-methoxyphenyl]cyclopropaneamido}-2H-pyrazol-3-yl)cyclopentyl N-isopropylcarbamate

Trans-2-[3-(benzyloxy)-2-(1,3-dioxolan-2-yl)-5-methoxyphenyl]cyclopropane-1-carboxylic acid (176.2 mg, 0.475 mmol), (1R,3S)-3-(5-amino-2H-pyrazol-3-yl)cyclopentyl N-isopropylcarbamate (100 mg, 0.396 mmol), a stir bar and pyridine (3 mL) were added to an oven-dried 50 mL round-bottom flask and stirred until homogenous, then treated with EDCI (152 mg, 0.792 mmol). The resulting mixture was stirred for 2 h at rt, then concentrated under vacuum. The residue was purified by reverse-phase chromatography (0-58% ACN/H₂O with 10 mM NH₄HCO₃) to afford (1R,3S)-3-(5-{trans-2-[3-(benzyloxy)-2-(1,3-dioxolan-2-yl)-5-methoxyphenyl]cyclopropaneamido}-2H-pyrazol-3-yl)cyclopentyl N-isopropylcarbamate (70 mg, 29.21%) as a yellow solid. MS (ESI) calcd. for C₃₃H₄₀N₄O₇, 604.29 m/a, found, 605.30 m/z [M+H]⁺.

### Step 4: Synthesis of (1R,3S)-3-{5-[trans-2-(2-formyl-3-hydroxy-5-methoxyphenyl)cyclo-propaneamido]-2H-pyrazol-3-yl}cyclopentyl N-isopropylcarbamate

(1R,3S)-3-{5-[trans-2-[3-(benzyloxy)-2-(1,3-dioxolan-2-yl)-5-methoxyphenyl]cyclopropane-amido]-2H-pyrazol-3-yl}cyclopentyl N-isopropylcarbamate (70 mg, 0.116 mmol), methane-sulfonic acid (1 mL), a stir bar and TFA (5 mL) were added to an oven-dried 50 mL round bottom flask and stirred until homogenous. The resulting mixture was stirred at rt for 30 min, then concentrated under vacuum. The residue was purified by Prep-HPLC with (Column: Xselect CSH Fluoro Phenyl 30×150 mm, 5 μm; Mobile Phase A: Water (0.05% TFA), Mobile Phase B: ACN; Flow rate: 60 mL/min; Gradient: 22% B to 44% B in 10 min; Wave Length: 254/220 nm; RT1 (min): 9.5) to afford (1R,3S)-3-{5-[trans-2-(2-formyl-3-hydroxy-5-methoxyphenyl) cyclopropaneamido]-2H-pyrazol-3-yl}cyclopentyl N-isopropylcarbamate (2 mg, 3.59%) as a white solid. MS (ESI) calcd. for C₂₄H₃₀N₄O₆, 470.22, found, 471.25[M+H]⁺. ¹H NMR (400 MHz, DMSO-d₆) δ 12.15 (s, 1H), 10.66 (s, 1H), 10.23 (s, 1H), 6.97 (d, J=7.0 Hz, 1H), 6.48-6.21 (m, 3H), 4.99 (s, 1H), 3.84 (s, 3H), 3.10-2.99 (m, 1H), 2.82 (d, J=10.2 Hz, 1H), 2.68 (d, J=2.0 Hz, 1H), 2.45 (s, 1H), 2.31 (d, J=3.8 Hz, 2H), 2.17 (d, J=8.3, 4.5 Hz, 1H), 2.05-1.95 (m, 1H), 1.94-1.82 (m, 1H), 1.72 (d, J=11.1 Hz, 1H), 1.52 (d, J=7.0 Hz, 1H), 1.47-1.39 (m, 1H), 1.38-1.31 (m, 1H), 1.10-0.93 (m, 6H).

### Step 5: Synthesis of (1R,3S)-3-{5-[(1S,2S)-2-(2-formyl-3-hydroxy-5-methoxyphenyl)cyclopropaneamido]-2H-pyrazol-3-yl}cyclopentyl N-isopropylcarbamate

(1R,3S)-3-{5-[trans-2-(2-formyl-3-hydroxy-5-methoxyphenyl) cyclopropaneamido]-2H-pyrazol-3-yl}cyclopentyl N-isopropylcarbamate was further separated by Prep-Chiral-HPLC with (Column: CHIRALPAK IF, 2×25 cm, 5 μm; Mobile Phase A: Hex:DCM=1:1 with 0.5% 2M NH₃-MeOH, Mobile Phase B: EtOH; Flow rate: 20 mL/min; Gradient: isocratic; Wave Length: 220/254 nm; RT1 (min): 8.855; RT2 (min): 10.384; Sample Solvent: EtOH; Injection Volume: 1.0 mL; Number Of Runs: 4) to afford two products. The second eluting was lyophilized to afford (1R,3S)-3-{5-[(1S,2S)-2-(2-formyl-3-hydroxy-5-methoxyphenyl)cyclopropaneamido]-2H-pyrazol-3-yl}cyclopentyl N-isopropylcarbamate (2.0 mg, 3.56%) as a white solid. MS (ESI) calcd. for C₂₄H₃₀N₄O₆, 470.22 m/a, found, 471.20 m/z [M+H]⁺. ¹H NMR (400 MHz, DMSO-d₆) δ 12.12 (s, 1H), 10.65 (s, 1H), 10.23 (s, 1H), 6.98 (d, J=7.7 Hz, 1H), 6.42-6.22 (m, 3H), 4.99 (s, 1H), 3.83 (s, 3H), 3.09-2.99 (m, 1H), 2.83 (s, 1H), 2.68 (d, J=2.3 Hz, 1H), 2.44 (s, 1H), 2.34-2.27 (m, 2H), 2.16 (s, 1H), 1.99 (s, 1H), 1.89 (s, 1H), 1.64 (d, J=48.8 Hz, 1H), 1.43 (s, 1H), 1.34 (s, 1H), 1.24 (s, 1H), 1.10-0.94 (m, 6H).

### Example 6.17: Synthesis of Compound 92: (1R,3S)-3-{5-[1-(2-formyl-3-hydroxyphenyl)piperidine-4-amido]-2H-pyrazol-3-yl}cyclopentyl N-isopropylcarbamate

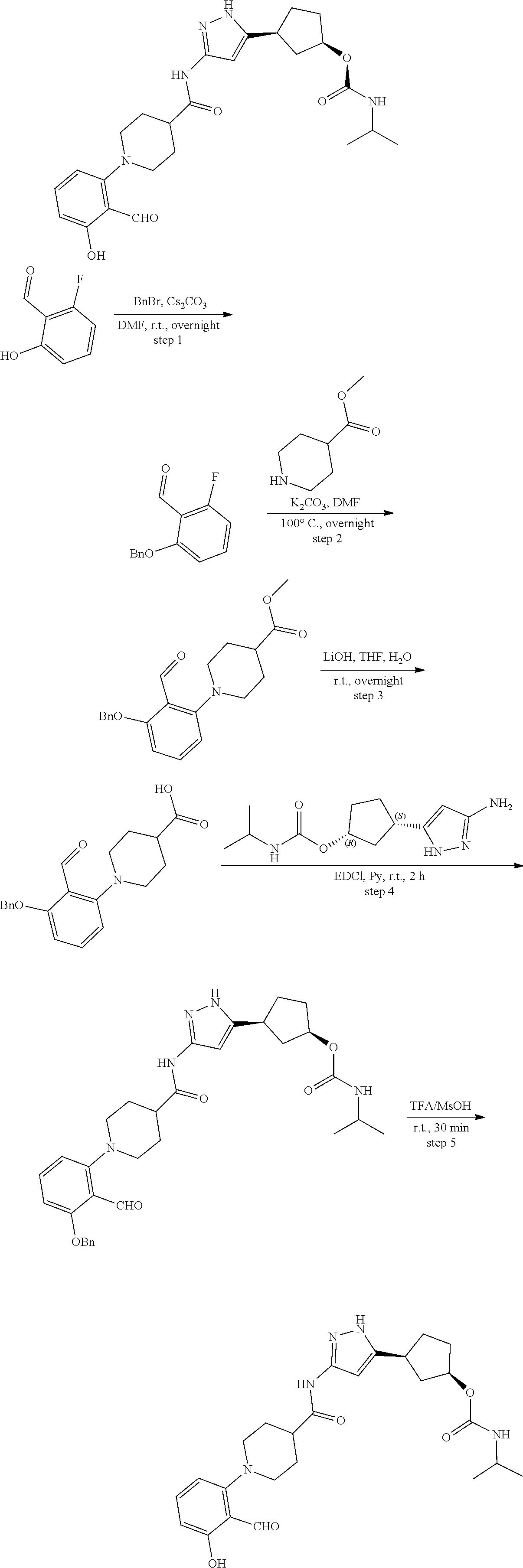

### Step 1: Synthesis of 2-(benzyloxy)-6-fluorobenzaldehyde

2-fluoro-6-hydroxybenzaldehyde (5 g, 35.685 mmol), Cs₂CO₃ (23.25 g, 71.370 mmol), a stir bar and DMF (50 mL) were added to an oven-dried 250 mL round bottom flask and stirred until homogenous, then treated with BnBr (9.16 g, 53.528 mmol). The resulting mixture was stirred overnight at rt, then quenched with NH₄Cl (20 mL) solution at 0° C. The resulting mixture was extracted with EA (100 mL) three times. The combined organic layers were washed with brine (10 mL), dried over anhydrous sodium sulfate, filtered, and concentrated under vacuum. The residue was purified by silica gel chromatography (0-81% PE/EA) to afford 2-(benzyloxy)-6-fluorobenzaldehyde (8 g, 97.37%) as a yellow oil. MS (ESI) calcd. for C₁₄H₁₁FO₂, 230.07, found, 231.15 [M+H]⁺.

### Step 2: Synthesis of methyl 1-[3-(benzyloxy)-2-formylphenyl]piperidine-4-carboxylate

2-(benzyloxy)-6-fluorobenzaldehyde (1.5 g, 6.515 mmol), methyl piperidine-4-carboxylate (1.4 g, 9.772 mmol), K₂CO₃ (2.25 g, 16.287 mmol), a stir bar and DMF (15 mL) were added to an oven-dried 100 mL round bottom flask and stirred until homogenous. The resulting mixture was stirred overnight at 100° C., then cooled to rt and quenched with H₂O (20 mL). The resulting mixture was extracted with EA (50 mL) three times. The combined organic layers were washed with brine (10 mL), dried over anhydrous sodium sulfate, filtered, and concentrated under vacuum. The residue was purified by silica gel chromatography (0-20% PE/EA) to afford methyl 1-[3-(benzyloxy)-2-formylphenyl]piperidine-4-carboxylate (2 g, 86.86%) as a yellow oil. MS (ESI) calcd. for C₂₁H₂₃NO₄, 353.16 m/z, found, 354.15 m/z [M+H]⁺.

### Step 3: Synthesis of 1-[3-(benzyloxy)-2-formylphenyl]piperidine-4-carboxylic acid

Methyl 1-[3-(benzyloxy)-2-formylphenyl]piperidine-4-carboxylate (2 g, 5.659 mmol), lithium hydroxide (0.41 g, 16.977 mmol), MeOH (4 mL), H₂O (4 mL), a stir bar and THF (12 mL) were added to an oven-dried 100 mL round-bottom flask and stirred until homogenous. The resulting mixture was stirred overnight at rt, then concentrated under vacuum. The pH of the mixture was adjusted to around pH 4 with 10 wt % HOAc solution. The resulting mixture was extracted with EA (50 mL) for three times. The combined organic layers were washed with brine (10 mL), dried over anhydrous sodium sulfate, filtered and concentrated under vacuum to afford 1-[3-(benzyloxy)-2-formylphenyl]piperidine-4-carboxylic acid (1.8 g, 93.72%) as a yellow solid. MS (ESI) calcd. for C₂₀H₂₁NO₄, 339.15 m/a, found, 340 m/a [M+H]⁺.

### Step 4: Synthesis of (1R,3S)-3-(5-{1-[3-(benzyloxy)-2-formylphenyl]piperidine-4-amido}-2H-pyrazol-3-yl)cyclopentyl N-isopropylcarbamate

1-[3-(benzyloxy)-2-formylphenyl]piperidine-4-carboxylic acid (148 mg, 0.436 mmol), (1R,3S)-3-(5-amino-2H-pyrazol-3-yl)cyclopentyl N-isopropylcarbamate (100 mg, 0.396 mmol), a stir bar and pyridine (3 mL) were added to an oven-dried 50 mL round-bottom flask and stirred until homogenous, then treated with EDCI (152 mg, 0.792 mmol). The resulting mixture was stirred for 2 h at rt, then concentrated under vacuum. The residue was purified by reverse-phase chromatography (0-56% ACN/H₂O with 10 mM NH₄HCO₃) to afford (1R,3S)-3-(5-{1-[3-(benzyloxy)-2-formylphenyl]piperidine-4-amido}-2H-pyrazol-3-yl)cyclopentyl N-isopropylcarbamate (20 mg, 8.80%) as a yellow solid. MS (ESI) calcd. for C₃₂H₃₉N₅O₅, 573.30, found, 574 [M+H]⁺.

### Step 5: Synthesis of (1R,3S)-3-{5-[1-(2-formyl-3-hydroxyphenyl)piperidine-4-amido]-2H-pyrazol-3-yl}cyclopentyl N-isopropylcarbamate

(1R,3S)-3-(5-{1-[3-(benzyloxy)-2-formylphenyl]piperidine-4-amido}-2H-pyrazol-3-yl)cyclopentyl N-isopropylcarbamate (20 mg, 0.035 mmol), methanesulfonic acid (0.5 mL), a stir bar and TFA (2.5 mL) were added to an oven-dried 50 mL round bottom flask and stirred until homogenous. The resulting mixture was stirred at rt for 30 min, then concentrated under vacuum. The residue was purified by Prep-HPLC with (Column: Xselect CSH Fluoro Phenyl 30×150 mm, 5 μm; Mobile Phase A: Water (0.05% TFA), Mobile Phase B: ACN; Flow rate: 60 mL/min; Gradient: 22% B to 44% B in 10 min; Wave Length: 254/220 nm; RT1 (min): 8.6) to afford (1R,3S)-3-{5-[1-(2-formyl-3-hydroxyphenyl)piperidine-4-amido]-2H-pyrazol-3-yl}cyclopentyl N-isopropylcarbamate (5.1 mg, 30.04%) as a yellow solid. MS (ESI) calcd. for C₂₅H₃₃N₅O₅, 483.25 m/a, found, 484.30 m/a [M+H]⁺. ¹H NMR (400 MHz, DMSO-d₆) δ 11.63 (s, 1H), 10.33 (s, 1H), 10.11 (s, 1H), 7.49 (t, J=8.2 Hz, 1H), 6.97 (d, J=7.9 Hz, 1H), 6.62 (d, J=33.3, 8.2 Hz, 2H), 6.32 (s, 1H), 4.99 (s, 1H), 3.58 (d, J=7.5 Hz, 1H), 3.33 (d, J=11.7 Hz, 2H), 3.04 (s, 1H), 2.87 (d, J=16.4 Hz, 2H), 2.36-2.28 (m, 1H), 2.04-1.95 (m, 1H), 1.93-1.79 (m, 5H), 1.77-1.52 (m, 4H), 1.07-0.98 (m, 6H).

### Example 6.18: Synthesis of Compound 93: (1R,3S)-3-{5-[1-(2-formyl-3-hydroxyphenyl)pyrrolidine-3-amido]-2H-pyrazol-3-yl}cyclopentyl N-isopropylcarbamate

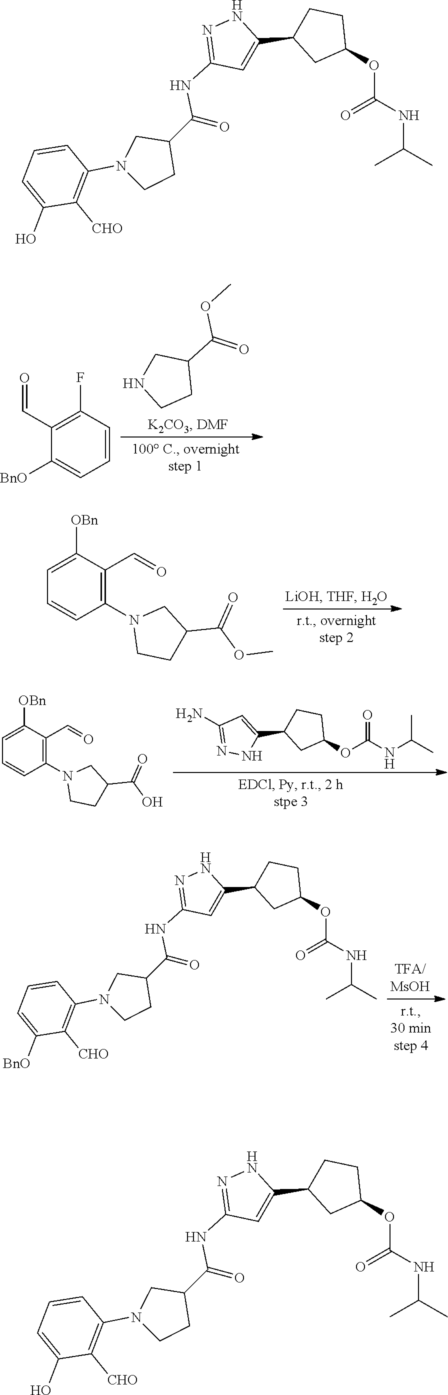

### Step 1: Synthesis of methyl 1-[3-(benzyloxy)-2-formylphenyl]pyrrolidine-3-carboxylate

2-(benzyloxy)-6-fluorobenzaldehyde (1.5 g, 6.515 mmol), methyl pyrrolidine-3-carboxylate hydrochloride (1.62 g, 9.772 mmol), K₂CO₃ (3.6 g, 26.060 mmol), a stir bar and DMF (15 mL) were added to an oven-dried 100 mL round bottom flask and stirred until homogenous. The resulting mixture was stirred overnight at 100° C., then cooled to room temperature and diluted with water. The resulting mixture was extracted with EA (50 mL) for three times. The combined organic layers were washed with brine (10 mL), dried over anhydrous sodium sulfate, filtered, and concentrated under vacuum. The residue was purified by silica gel chromatography (0-20% PE/EA) to afford methyl 1-[3-(benzyloxy)-2-formylphenyl]pyrrolidine-3-carboxylate (1.3 g, 58.79%) as a yellow oil. MS (ESI) calcd. for C₂₀H₂₁NO₄, 339.15 m/z, found, 340.15 m/z [M+H]⁺.

### Step 2: Synthesis of 1-[3-(benzyloxy)-2-formylphenyl]pyrrolidine-3-carboxylic acid

Methyl 1-[3-(benzyloxy)-2-formylphenyl]pyrrolidine-3-carboxylate (1.3 g, 3.830 mmol), lithium hydroxide (0.28 g, 11.490 mmol), MeOH (3 mL), H₂O (3 mL), a stir bar and THF (9 mL) were added to an oven-dried 100 mL round-bottom flask and stirred until homogenous. The resulting mixture was stirred overnight at rt, then concentrated under vacuum. The pH of the mixture was adjusted to around 4 with 10 wt % HOAc solution. The resulting mixture was extracted with EA (50 mL) for three times. The combined organic layers were washed with brine (10 mL), dried over anhydrous sodium sulfate, filtered and concentrated under vacuum to afford 1-[3-(benzyloxy)-2-formylphenyl]pyrrolidine-3-carboxylic acid (1.2 g, 96.29%) as a yellow oil. MS (ESI) calcd. for C₁₉H₁₉NO₄, 325.13 m/z, found, 326.00 m/z [M+H]⁺.

### Step 3: Synthesis of (1R,3S)-3-(5-{1-[3-(benzyloxy)-2-formylphenyl]pyrrolidine-3-amido}-2H-pyrazol-3-yl)cyclopentyl N-isopropylcarbamate

1-[3-(benzyloxy)-2-formylphenyl]pyrrolidine-3-carboxylic acid (218 mg, 0.653 mmol), (1R,3S)-3-(5-amino-2H-pyrazol-3-yl)cyclopentyl N-isopropylcarbamate (150 mg, 0.594 mmol), a stir bar and pyridine (3 mL) were added to an oven-dried 50 mL round bottom flask and stirred until homogenous, then treated with EDCI (228 mg, 1.188 mmol). The resulting mixture was stirred for 2 h at rt, then concentrated under vacuum. The residue was purified by reverse-phase chromatography (0-52% ACN/H₂O with 10 mM NH₄HCO₃) to afford (1R,3S)-3-(5-{1-[3-(benzyloxy)-2-formylphenyl]pyrrolidine-3-amido}-2H-pyrazol-3-yl)cyclopentyl N-isopropylcarbamate (30 mg, 9.02%) as a yellow solid. MS (ESI) calcd. for C₃₁H₃₇N₅O₅, 559.28 m/z, found, 560 m/z [M+H]⁺.

### Step 4: Synthesis of (1R,3S)-3-{5-[1-(2-formyl-3-hydroxyphenyl)pyrrolidine-3-amido]-2H-pyrazol-3-yl}cyclopentyl N-isopropylcarbamate

(1R,3S)-3-(5-{1-[3-(benzyloxy)-2-formylphenyl]pyrrolidine-3-amido}-2H-pyrazol-3-yl)cyclopentyl N-isopropylcarbamate (30 mg, 0.054 mmol), methanesulfonic acid (0.5 mL), a stir bar and TFA (2.5 mL) were added to an oven-dried 50 mL round bottom flask and stirred until homogenous. The resulting mixture was stirred at rt for 30 min, then concentrated under vacuum. The residue was purified by Prep-HPLC with (Column: Xselect CSH Fluoro Phenyl 30×150 mm, 5 μm; Mobile Phase A: Water (0.05% TFA), Mobile Phase B: ACN; Flow rate: 60 mL/min; Gradient: 25% B to 45% B in 10 min; Wave Length: 254/220 nm; RT1 (min): 8.1) to afford (1R,3S)-3-{5-[1-(2-formyl-3-hydroxyphenyl)pyrrolidine-3-amido]-2H-pyrazol-3-yl}cyclopentyl N-isopropylcarbamate (6.4 mg, 22.40%) as a yellow solid. MS (ESI) calcd. for C₂₄H₃₁N₅O₅, 469.23, found, 470.25 [M+H]⁺. ¹H NMR (400 MHz, DMSO-d₆) δ 11.76 (s, 1H), 10.50 (s, 1H), 10.17 (s, 1H), 7.28 (t, J=8.3 Hz, 1H), 7.01-6.91 (m, 1H), 6.39-6.30 (m, 2H), 6.23 (d, J=8.0 Hz, 1H), 4.99 (s, 1H), 3.45-3.37 (m, 2H), 3.30-3.14 (m, 2H), 3.08-2.99 (m, 1H), 2.37-2.28 (m, 1H), 2.22-2.06 (m, 2H), 1.94 (dd, J=47.7, 6.0 Hz, 2H), 1.75-1.51 (m, 3H), 1.09-0.93 (m, 6H).

### Example 6.19: Synthesis of Compound 96: (1R,3S)-3-(5-{2-[(2-formyl-3-hydroxyphenyl)sulfanyl]acetamido}-2H-pyrazol-3-yl)cyclopentyl N-isopropylcarbamate; trifluoroacetic acid

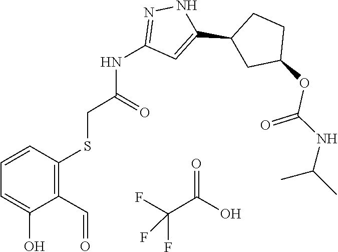 [SMILES: CC(C)NC(=O)O[C@@H]1CC[C@H](c2cc(NC(=O)CSc3cccc(O)c3C=O)n[nH]2)C1 ; O=C(O)C(F)(F)F]

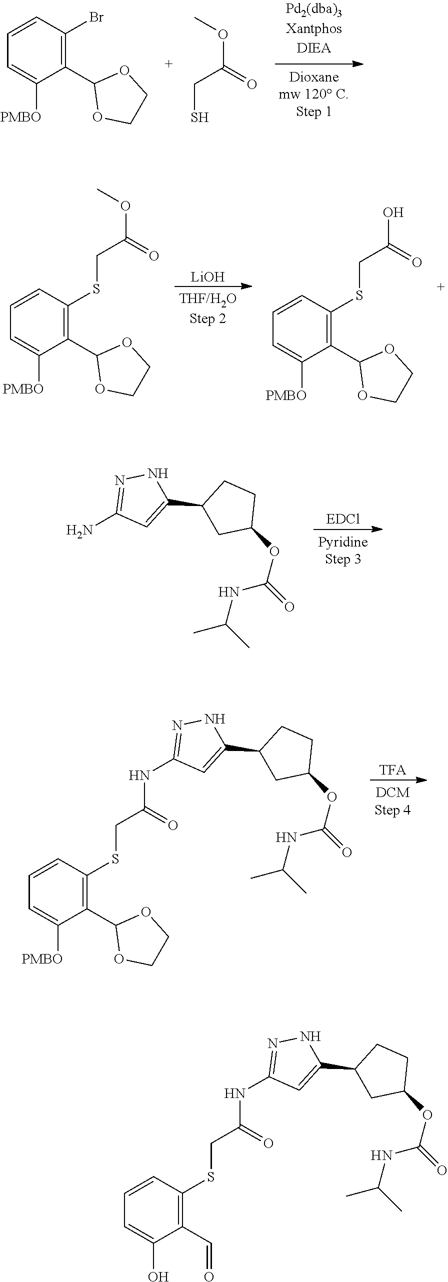

### Step 1: Synthesis of methyl 2-{[2-(1,3-dioxolan-2-yl)-3-[(4-methoxyphenyl)methoxy]phenyl]sulfanyl}acetate

To a stirred mixture of 2-{2-bromo-6-[(4-methoxyphenyl)methoxy]phenyl}-1,3-dioxolane (1.0 g, 2.738 mmol, 1 equiv) and methyl thioglycolate (0.29 g, 2.738 mmol, 1 equiv) in dioxane (20 mL, 226.999 mmol) were added Pd₂(dba)₃ (0.25 g, 0.274 mmol, 0.1 equiv), Xantphos (0.16 g, 0.274 mmol, 0.1 equiv) and DIEA (0.88 g, 2.5 equiv) at 25° C. under N₂ atmosphere. The final reaction mixture was irradiated with microwave radiation for 1 h at 120° C. The reaction was monitored by LCMS. The resulting mixture was concentrated under reduced pressure. The residue was purified by silica gel column chromatography, eluted with EA/PE (30%) to afford methyl 2-{[2-(1,3-dioxolan-2-yl)-3-[(4-methoxyphenyl)methoxy]phenyl]sulfanyl}acetate (1.0 g, 93.54%) as a yellow oil. MS (ESI) calcd. for C₂₀H₂₂O₆S, 390.11 m/z, found 391.15 [M+H]⁺.

### Step 2: Synthesis of {[2-(1,3-dioxolan-2-yl)-3-[(4-methoxyphenyl)methoxy]phenyl]sulfanyl}acetic acid

To a stirred solution of methyl 2-{[2-(1,3-dioxolan-2-yl)-3-[(4-methoxyphenyl)methoxy]phenyl]sulfanyl}acetate (500.0 mg, 1.281 mmol, 1 equiv) in THF (5 mL) and H₂O (5 mL) were added LiOH (153.35 mg, 6.405 mmol, 5 equiv) at 25° C. The resulting mixture was stirred for 1 h at 25° C. The reaction was monitored by LCMS. The residue was neutralized to pH 7-8 with citric acid. The resulting mixture was extracted with EA (3×30 mL). The combined organic layers were washed with brine (3×30 mL), dried over anhydrous Na₂SO₄. After filtration, the filtrate was concentrated under reduced pressure. This resulted in {[2-(1,3-dioxolan-2-yl)-3-[(4-methoxyphenyl)methoxy]phenyl]sulfanyl}acetic acid (400.0 mg, 82.98%) as a yellow oil. MS (ESI) calcd. for C₁₉H₂₀O₆S, 376.10 m/z, found 377.15 [M+H]⁺.

### Step 3: Synthesis of (1R,3S)-3-[5-(2-{[2-(1,3-dioxolan-2-yl)-3-[(4-methoxyphenyl)methoxy]phenyl]sulfanyl}acetamido)-2H-pyrazol-3-yl]cyclopentyl N-isopropylcarbamate

To a stirred solution of {[2-(1,3-dioxolan-2-yl)-3-[(4-methoxyphenyl)methoxy]phenyl]sulfanyl}acetic acid (200.0 mg, 0.531 mmol, 1 equiv) and (1R,3S)-3-(5-amino-2H-pyrazol-3-yl)cyclopentyl N-isopropylcarbamate (134.1 mg, 0.531 mmol, 1 equiv) in pyridine (10 mL) were added EDCI (407.4 mg, 2.124 mmol, 4 equiv) at 25° C. The resulting mixture was stirred for 1.5 h at 25° C. The reaction was monitored by LCMS. The resulting mixture was diluted with water and extracted with EA (3×20 mL). The combined organic layers were washed with brine (3×30 mL), dried over anhydrous Na₂SO₄. After filtration, the filtrate was concentrated under reduced pressure. The crude resulting mixture was used in the next step directly without further purification. MS (ESI) calcd. for C₃₁H₃₈N₄O₇S, 610.25 m/z, found 611.35 [M+H]⁺.

### Step 4: Synthesis of (1R,3S)-3-(5-{2-[(2-formyl-3-hydroxyphenyl)sulfanyl]acetamido}-2H-pyrazol-3-yl)cyclopentyl N-isopropylcarbamate; trifluoroacetic acid

To a stirred solution of (1R,3S)-3-[5-(2-{[2-(1,3-dioxolan-2-yl)-3-[(4-methoxyphenyl)methoxy]phenyl]sulfanyl}acetamido)-2H-pyrazol-3-yl]cyclopentyl N-isopropylcarbamate (100 mg, 0.164 mmol, 1 equiv) in DCM (3 mL) was added TFA (1 mL) dropwise at 25° C. The resulting mixture was stirred for 1 h at 25° C. The resulting mixture was concentrated under reduced pressure. The crude product was purified by Prep-HPLC with the following conditions (Column: Xselect CSH Prep C18 OBD Column, 30×150 mm, 5 um; Mobile Phase A: Water (0.05% TFA), Mobile Phase B: ACN; Flow rate: 60 mL/min; Gradient: 25% B to 47% B in 10 min; Wave Length: 254/220 nm) to afford (1R,3S)-3-(5-{2-[(2-formyl-3-hydroxyphenyl)sulfanyl]acetamido}-2H-pyrazol-3-yl)cyclopentyl N-isopropylcarbamate; trifluoroacetic acid (9.5 mg, 10.23%) as a white solid. MS (ESI) calcd. for C₂₃H₂₇F₃N₄O₇S, 446.16 m/z, found 447.20 [M+H]⁺. ¹H NMR (400 MHz, DMSO-d₆) δ (ppm): 10.98 (br s, 1H), 10.63 (s, 1H), 10.47 (s, 1H), 7.42 (t, J=8.0 Hz, 1H), 6.96 (d, J=8.0 Hz, 2H), 6.77 (d, J=8.0 Hz, 1H), 6.28 (s, 1H), 4.98 (br s, 1H), 3.81 (s, 2H), 3.53-3.59 (m, 1H), 3.01-3.07 (m, 1H), 2.42-2.47 (m, 1H), 1.96-2.00 (m, 1H), 1.83-1.92 (m, 1H), 1.53-1.72 (m, 3H), 1.01-1.03 (m, 6H). ¹⁹F NMR (376 MHz, DMSO-d₆) δ (ppm): −74.80.

### Example 6.20: Synthesis of Compound 69: (1R,3S)-3-{5-[2-(2-formyl-3-hydroxyphenoxy)pyridine-4-amido]-2H-pyrazol-3-yl}cyclopentyl N-isopropylcarbamate; trifluoroacetic acid

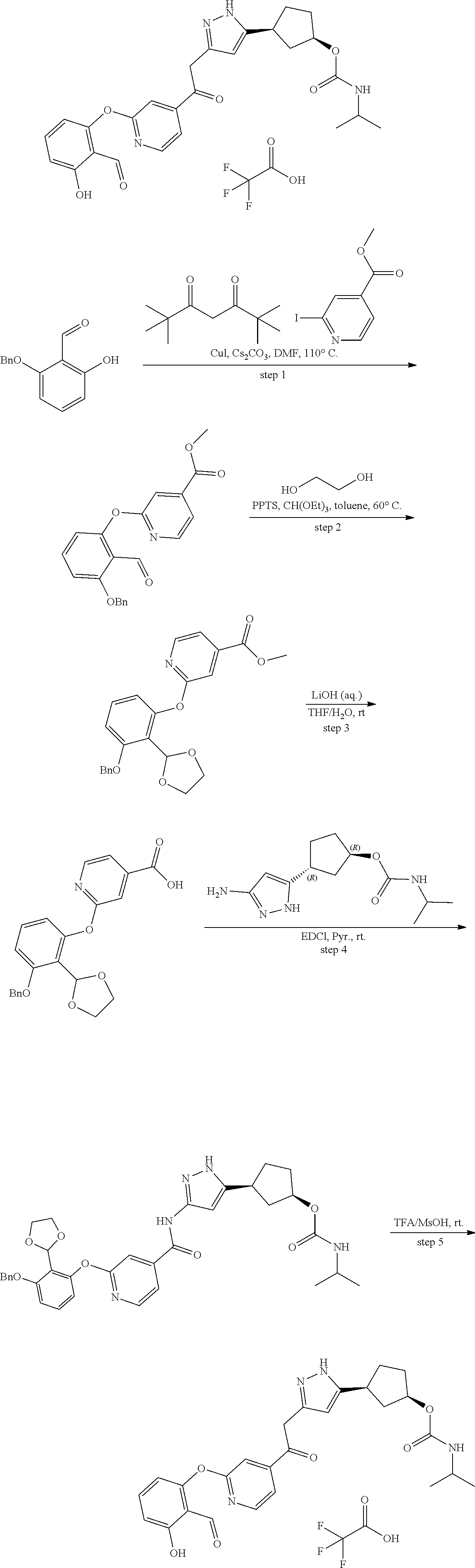

### Step 1: Synthesis of methyl 2-[3-(benzyloxy)-2-formylphenoxy]pyridine-4-carboxylate

To a stirred solution of 2-(benzyloxy)-6-hydroxybenzaldehyde (2.0 g, 8.762 mmol, 1 equiv), methyl 2-iodopyridine-4-carboxylate (2.8 g, 10.514 mmol, 1.2 equiv) in anhydrous DMF (20 mL) was added Cs₂CO₃ (7.1 g, 21.905 mmol, 2.5 equiv) and 2,2,6,6-tetramethylheptane-3,5-dione (0.5 g, 2.629 mmol, 0.3 equiv) followed by catalytic amount of CuI (0.3 g, 1.752 mmol, 0.2 equiv) at rt. The reaction mixture was stirred at 110° C. for a period of 2 h. Then the resulting mixture was cooled to room temperature, diluted with water and extracted with EA (3×100 mL). The combined organic layers were washed with brine (3×100 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O (with 5% NH₄HCO₃)/ACN, 10% to 95% gradient in 30 min; detector, UV 254 nm. This resulted in methyl 2-[3-(benzyloxy)-2-formylphenoxy]pyridine-4-carboxylate (600 mg, 18.84%) as a brown solid. MS (ESI) calcd. for C₂₁H₁₇NO₅, 363.11 m/z, found 364.05 [M+H]⁺.

### Step 2: Synthesis of methyl 2-(3-(benzyloxy)-2-(1,3-dioxolan-2-yl)phenoxy)isonicotinate

To a stirred solution of methyl 2-[3-(benzyloxy)-2-formylphenoxy]pyridine-4-carboxylate (500.0 mg, 1.376 mmol, 1 equiv) in anhydrous toluene (10 mL) was added triethyl orthoformate (611.8 mg, 4.128 mmol, 3 equiv) and ethylene glycol (427.0 mg, 6.880 mmol, 5 equiv) followed by catalytic amount of PPTS (23.7 mg, 0.138 mmol, 0.1 equiv) at rt. The reaction mixture was stirred at 60° C. for a period of 12 h. The resulting mixture was cooled to room temperature, diluted with water and extracted with EA (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (3/1) to afford methyl 2-[3-(benzyloxy)-2-(1,3-dioxolan-2-yl)phenoxy]pyridine-4-carboxylate (550.0 mg, 98.11%) as a yellow solid. MS (ESI) calcd. for C₂₃H₂₁NO₆, 407.14 m/z, found 408.05 [M+H]⁺.

### Step 3: Synthesis of 2-[3-(benzyloxy)-2-(1,3-dioxolan-2-yl)phenoxy]pyridine-4-carboxylic acid

To a stirred solution of methyl 2-[3-(benzyloxy)-2-(1,3-dioxolan-2-yl)phenoxy]pyridine-4-carboxylate (300.0 mg, 0.736 mmol, 1 equiv) in anhydrous THF (3 mL), H₂O (3 mL) was added LiOH (52.9 mg, 2.208 mmol, 3 equiv) at room temperature and stirred for 1 h. The mixture was acidified to pH 5 with HCl (0.5 M). The precipitated solids were collected by filtration and washed with H₂O (3×20 mL). This resulted in 2-[3-(benzyloxy)-2-(1,3-dioxolan-2-yl)phenoxy]pyridine-4-carboxylic acid (251.0 mg, 86.65%) as a white solid. MS (ESI) calcd. for C₂₂H₁₉NO₆, 393.12 m/z, found 394.05 [M+H]⁺.

### Step 4: Synthesis of (1R,3S)-3-(5-{2-[3-(benzyloxy)-2-(1,3-dioxolan-2-yl)phenoxy]pyridine-4-amido}-2H-pyrazol-3-yl)cyclopentyl N-isopropylcarbamate

To a stirred solution of 2-[3-(benzyloxy)-2-(1,3-dioxolan-2-yl)phenoxy]pyridine-4-carboxylic acid (200.0 mg, 0.508 mmol, 1 equiv) and (1R,3S)-3-(5-amino-2H-pyrazol-3-yl)cyclopentyl N-isopropylcarbamate (128.3 mg, 0.508 mmol, 1 equiv) in anhydrous pyridine (5 mL) was added EDCI (117.0 mg, 0.610 mmol, 1.2 equiv). The reaction mixture was stirred at room temperature for a period of 1 h. The resulting mixture was diluted with water and extracted with EA (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (3/1) to afford (1R,3S)-3-(5-{2-[3-(benzyloxy)-2-(1,3-dioxolan-2-yl)phenoxy]pyridine-4-amido}-2H-pyrazol-3-yl)cyclopentyl N-isopropylcarbamate (80.0 mg, 14.95%) as a yellow solid. MS (ESI) calcd. for C₃₄H₃₇N₅O₇, 393.12 m/z, found 394.05 [M+H]⁺.

### Step 5: Synthesis of (1R,3S)-3-{5-[2-(2-formyl-3-hydroxyphenoxy)pyridine-4-amido]-2H-pyrazol-3-yl}cyclopentyl N-isopropylcarbamate; trifluoroacetic acid

To a stirred solution of (1R,3S)-3-(5-{2-[3-(benzyloxy)-2-(1,3-dioxolan-2-yl)phenoxy]pyridine-4-amido}-2H-pyrazol-3-yl)cyclopentyl N-isopropylcarbamate (70.0 mg, 0.112 mmol, 1 equiv) in TFA (1.5 mL) were added MsOH (0.5 mL) dropwise at room temperature. The resulting mixture was stirred for 0.5 h. The reaction was monitored by LCMS. The resulting mixture was concentrated under reduced pressure. The crude product was purified by Prep-HPLC with the following conditions (Column: XBridge Shield RP18 OBD Column, 30×150 mm, 5 um; Mobile Phase A: Water (0.05% TFA), Mobile Phase B: ACN; Flow rate: 60 mL/min; Gradient: 29% B to 51% B in 10 min; Wave Length: 254 nm/220 nm; RT1 (min): 9.5) to afford (1R,3S)-3-{5-[2-(2-formyl-3-hydroxyphenoxy)pyridine-4-amido]-2H-pyrazol-3-yl}cyclopentyl N-isopropyl carbamate; trifluoroacetic acid (10.8 mg, 15.72%) as yellow solid. MS(ESI) calcd for C₂₇H₂₈F₃N₅O₈, 493.20 m/z, found 494.25 [M+H]⁺. ¹H NMR (400 MHz, DMSO-d₆) δ 11.30 (br s, 1H), 11.12 (s, 1H), 10.21 (s, 1H), 8.28 (s, 1H), 7.57-7.59 (m, 3H), 6.87-6.98 (m, 3H), 6.47-6.69 (m, 1H), 5.01-5.02 (m, 1H), 3.54-3.60 (m, 1H), 3.07-3.12 (m, 1H), 2.44-2.46 (m, 1H), 2.01-2.07 (m, 1H), 1.90-1.95 (m, 1H), 1.73-1.87 (m, 2H), 1.62-1.66 (m, 1H), 1.02-1.04 (m, 6H). ¹⁹F NMR (376 MHz, DMSO-d₆) δ −74.91.

### Example 6.21: Compound 107 (1R,3S)-3-(3-((1r,3S)-3-(2-formyl-3-hydroxyphenyl)cyclobutane-1-carboxamido)-1H-pyrazol-5-yl)cyclopentyl isopropylcarbamate

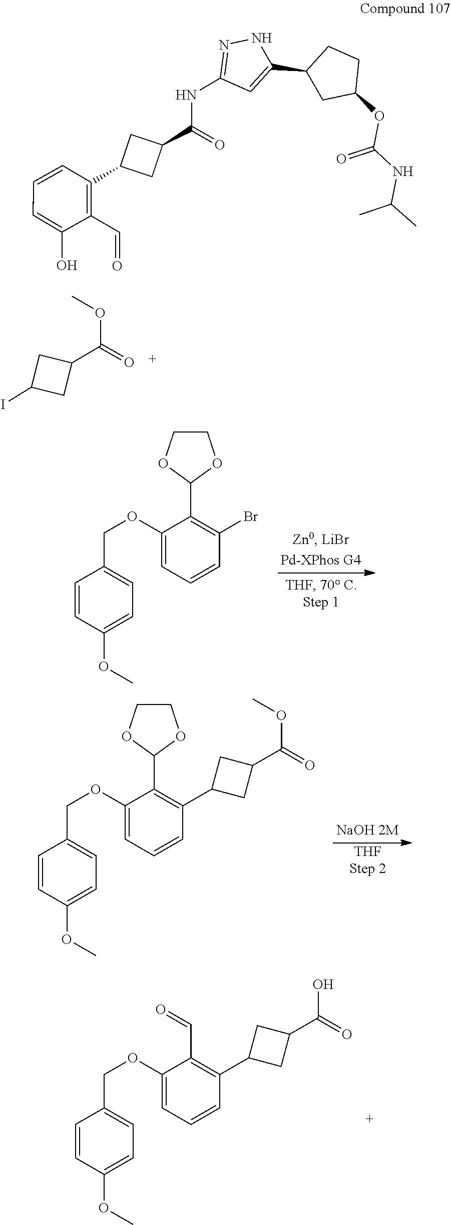

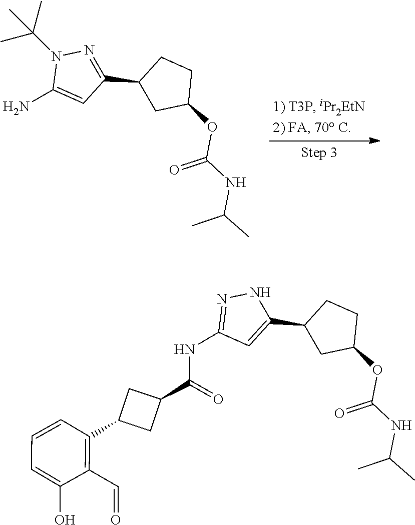 [SMILES: CC(C)NC(=O)O[C@@H]1CC[C@H](c2cc(N)n(C(C)(C)C)n2)C1 ; CC(C)NC(=O)O[C@@H]1CC[C@H](c2cc(NC(=O)[C@H]3C[C@H](c4cccc(O)c4C=O)C3)n[nH]2)C1]

### Step 1: Synthesis of methyl 3-(2-(1,3-dioxolan-2-yl)-3-((4-methoxybenzyl)oxy)phenyl) cyclobutane-1-carboxylate

An oven dried 40 mL vial equipped with a stir bar under an atmosphere of nitrogen was charged with methyl 3-iodocyclobutane-1-carboxylate (300 mg, 1.25 mmol, 1 equiv.), anhydrous LiBr (217 mg, 2.5 mmol, 2 euiv) and 5 mL THF. Rieke Zinc, 0.05 g/mL solution in THF, (3.3 mL, 2.5 mmol, 2 equiv,) was then added dropwise and let stir for 2 h at room temperature. After 2 h, under a stream of nitrogen 2-(2-bromo-6-((4-methoxybenzyl)oxy)phenyl)-1,3-dioxolane (411 mg, 1.12 mmol, 0.9 equiv) along with Pd-XPhos G4 (108 mg, 0.125 mmol, 0.1 equiv) were added portion-wise. The reaction vessel was sealed and heated at 70° C. for 1 h, the reaction was checked by LCMS and showed complete consumption of starting material. The crude reaction was quenched with 5 mL of a saturated solution of ammonium chloride and further diluted with 2 mL water to fully dissolve all salts. The organic phase was separated, and the aqueous phase was extracted three times with 5 mL EtOAc. The combined organic extracts were dried over sodium sulphate, filtered, and concentrated to dryness. The crude material was purified by silica gel column chromatography, 0 to 100% EtOAc/hex to provide methyl 3-(2-(1,3-dioxolan-2-yl)-3-((4-methoxybenzyl)oxy)phenyl) cyclobutane-1-carboxylate (185 mg, 37% yield).

### Step 2: Synthesis of 3-(2-formyl-3-((4-methoxybenzyl)oxy)phenyl)cyclobutane-1-carboxylic acid

A 40 mL vial was charged with methyl 3-(2-(1,3-dioxolan-2-yl)-3-((4-methoxybenzyl)oxy)phenyl) cyclobutane-1-carboxylate (185 mg, 0.53 mmol, 1 equiv) 5 mL THF and 0.5 mL 2 M NaOH. The reaction mixture was heated at 60° C. for 3 h until complete consumption of starting material. The reaction mixture was cooled to room temperature and acidified to pH 3 with 1 N HCl. The organics were separated, and the aqueous phase was extracted 3 times with 10 mL EtOAc. The combined organics were dried over sodium sulphate, filtered, and rotovapped to dryness to afford 3-(2-formyl-3-((4-methoxybenzyl)oxy)phenyl)cyclobutane-1-carboxylic acid (160 mg, 90% Yield). The crude material was used without any further purification.

### Step 3: Synthesis of (1R,3S)-3-(3-((1s,3R)-3-(2-formyl-3-hydroxyphenyl)cyclobutane-1-carboxamido)-1H-pyrazol-5-yl)cyclopentyl isopropylcarbamate and (1R,3S)-3-(3-((1r,3S)-3-(2-formyl-3-hydroxyphenyl)cyclobutane-1-carboxamido)-1H-pyrazol-5-yl)cyclopentyl isopropylcarbamate

A 40 mL vial was charged with 3-(2-formyl-3-((4-methoxybenzyl)oxy)phenyl)cyclobutane-1-carboxylic acid (135 mg, 0.4 mmol, 1 equiv), (1R,3S)-3-(5-amino-1-(tert-butyl)-1H-pyrazol-3-yl)cyclopentyl isopropylcarbamate (135 mg, 0.44 mmol, 1.1 equiv), 2 mL THF, followed by sequential dropwise addition of T3P 50% Solution in THF (0.7 mL, 1.2 mmol, 3 equiv) then diisopropylethyl amine (0.21 mL, 1.19 mmol, 3 equiv). The reaction mixture was let stir overnight at room temperature. The next day the reaction was quenched with 1 M NaOH (4 mL), the organic phase was separated and the aqueous phase was extracted 3 times with 5 mL EtOAc. The combined organic extracts were dried over sodium sulphate, and rotovapped to dryness. The crude material was heated in 1 ml formic acid at 70° C. for 8 h. The crude material was diluted with DMF and water, filtered through a syringe filter and purified by reverse phase HPLC, 10 to 70% MeCN/H₂O with 0.05% TFA modifier to afford (1R,3S)-3-(3-((1s,3R)-3-(2-formyl-3-hydroxyphenyl)cyclobutane-1-carboxamido)-1H-pyrazol-5-yl)cyclopentyl isopropylcarbamate (12 mg, 13% yield) ¹H NMR (400 MHz, DMSO) δ 11.44 (s, 1H), 10.34 (s, 1H), 10.26 (s, 1H), 7.52 (t, J=8.0 Hz, 1H), 6.91 (t, J=8.0 Hz, 2H), 6.84 (d, J=8.3 Hz, 1H), 6.30 (s, 1H), 4.97 (s, 1H), 4.06 (p, J=9.2 Hz, 1H), 3.10-2.93 (m, 1H), 2.36-2.21 (m, 2H), 2.04-1.94 (m, 1H), 1.94-1.80 (m, 1H), 1.77-1.49 (m, 3H), 1.02 (d, J=6.5 Hz, 6H), MS (ESI) calcd. for C₂₄H₃₀N₄O₅ 454.22 m/z, found [M+H]⁺ 455.35 m/z and (1R,3S)-3-(3-((1r,3S)-3-(2-formyl-3-hydroxyphenyl)cyclobutane-1-carboxamido)-1H-pyrazol-5-yl)cyclopentyl isopropylcarbamate (14 mg, 15% yield)¹H NMR (400 MHz, DMSO) δ 11.40 (s, 1H), 10.27 (s, 1H), 10.20 (s, 1H), 7.53 (t, J=8.0 Hz, 1H), 7.00 (d, J=7.7 Hz, 1H), 6.94 (d, J=7.8 Hz, 1H), 6.86 (d, J=8.3 Hz, 1H), 6.36 (s, 1H), 5.00 (s, 1H), 3.65-3.48 (m, 1H), 3.18 (dt, J=9.2, 4.3 Hz, 1H), 3.05 (t, J=8.4 Hz, 1H), 2.60 (ddd, J=11.8, 9.1, 4.9 Hz, 2H), 2.38-2.27 (m, 2H), 2.01 (q, J=7.8 Hz, 1H), 1.95-1.81 (m, 1H), 1.81-1.53 (m, 3H), 1.04 (d, J=6.6 Hz, 6H) MS (ESI) calcd. for C₂₄H₃₀N₄O₅ 454.22 m/z, found [M+H]⁺ 455.35 m/z

### Example 6.22: Synthesis of Compound 10: (1R,3S)-3-(3-((1S,2S)-2-(2-formyl-3-hydroxyphenyl)cyclopropane-1-carboxamido)-1H-pyrazol-5-yl)cyclopentyl isopropylcarbamate

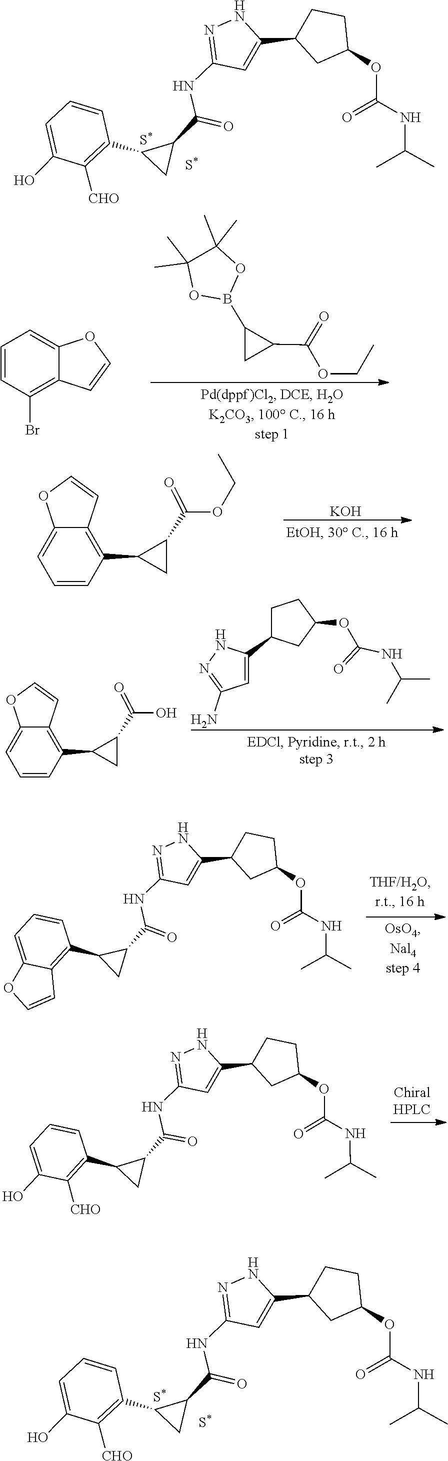

### Step 1: Synthesis of Ethyl trans-2-(1-benzofuran-4-yl)cyclopropane-1-carboxylate

4-bromo-1-benzofuran (500 mg, 0.508 mmol), ethyl 2-(4,4,5,5-tetramethyl-1,3,2-dioxaborolan-2-yl)cyclopropane-1-carboxylate (1.8 g, 3.81 mmol), K₂CO₃ (1.4 g, 8 mmol), Pd(dppf)Cl₂ (185.7 mg, 0.510 mmol), a stir bar and DCE (15 mL), H₂O (3 mL) were added to a 40 mL vail and stirred until homogenous. The resulting mixture was stirred overnight at 100° C. under nitrogen atmosphere, then cooled to room temperature and diluted with water. The resulting mixture was extracted with EA (80 mL×3). The combined organic layers were washed with brine, dried over anhydrous Na₂SO₄. After filtration, the filtrate was concentrated under reduced pressure. The residue was purified by silica gel column with PE/EA (0-20%) to afford two products. The second eluting was concentrated to afford ethyl trans-2-(1-benzofuran-4-yl)cyclopropane-1-carboxylate (400 mg, 51.34% yield) as a yellow oil and The first eluting was concentrated to afford ethyl cis-2-(1-benzofuran-4-yl)cyclopropane-1-carboxylate (250 mg, 29.95% yield) as a yellow oil. MS (ESI) mass calcd. for C₁₄H₁₄O₃, 230.09 m/z, found 231.10 [M+H]⁺.

### Step 2: Synthesis of trans-2-(1-benzofuran-4-yl)cyclopropane-1-carboxylic acid

Ethyl trans-2-(1-benzofuran-4-yl)cyclopropane-1-carboxylate (250 mg, 1.086 mmol), KOH (2.71 mL, 5.430 mmol, 2 M) and EtOH (15.00 mL) were added to a 100 mL round-bottom flask and stirred until homogeneous. The resulting mixture was stirred at 30° C. overnight. The mixture/residue was acidified to pH 5 with HCl (2 M). The resulting mixture was extracted with EA (3×60 mL). The combined organic layers were washed with brine, dried over anhydrous sodium sulfate, filtered and concentrated under vacuum to afford trans-2-(1-benzofuran-4-yl)cyclopropane-1-carboxylic acid as a white solid (210 mg, 95.65%). MS (ESI) calcd. for C₁₂H₁₀O₃, 202.06 m/z, found 201.00 [M−H]⁻.

### Step 3: Synthesis of trans-3-{5-[(1R,2R)-2-(1-benzofuran-4-yl)cyclopropaneamido]-2H-pyrazol-3-yl}cyclopentyl N-isopropylcarbamate

Trans-2-(1-benzofuran-4-yl)cyclopropane-1-carboxylic acid (200.1 mg, 1.236 mmol), (1R,3S)-3-(5-amino-2H-pyrazol-3-yl)cyclopentyl N-isopropylcarbamate (240 mg, 0.951 mmol) and pyridine (15 mL) were added to a 40 mL vail and stirred until homogeneous, then treated with EDCI (218.8 mg, 1.141 mmol). The reaction mixture was stirred at rt. for 2 h. The reaction was quenched with H₂O and extracted with EA. The combined organic layers were washed with brine, dried over anhydrous sodium sulfate, filtered, and concentrated under vacuum. The residue was purified by reverse-phase chromatography; 5-50%, ACN/water (5 mM NH₄HCO₃ modifier) to afford (1R,3S)-3-{5-[trans-2-(1-benzofuran-4-yl)cyclopropaneamido]-2H-pyrazol-3-yl}cyclopentyl N-isopropylcarbamate (190 mg, 45.76%) as a yellow solid. MS (ESI) mass calcd. for C₂₄H₂₈N₄O₄, 436.21 m/z, found 437.25 [M+H]⁺.

### Step 4: Synthesis of (1R,3S)-3-(3-((1S,2S)-2-(2-formyl-3-hydroxyphenyl)cyclopropane-1-carboxamido)-1H-pyrazol-5-yl)cyclopentyl isopropylcarbamate

(1R,3S)-3-{5-[trans-2-(1-benzofuran-4-yl)cyclopropaneamido]-2H-pyrazol-3-yl}cyclopentyl N-isopropylcarbamate (190 mg, 0.435 mmol), a stir bar, tetrahydrofuran (15 mL) and water (15 mL) were added to a 100 mL flask and stirred until homogenous, then treated with osmium tetroxide (442.6 mg, 0.044 mmol, 2.5% in t-BuOH) at 0° C. After stirring for 30 min at rt, sodium periodate (558.60 mg, 2.610 mmol) was added in several portions. The resulting mixture was stirred at rt. overnight. The reaction was quenched with H₂O and extracted with EA. The organic layers were combined, dried over Na₂SO₄, filtered and concentrated. The residue obtained was purified by Prep-HPLC (Column: Xselect CSH Prep C18 OBD Column, 30×150 mm, 5 um; Mobile Phase A: Water (0.1% FA), Mobile Phase B: ACN; Flow rate: 60 mL/min; Gradient: 26% B to 48% B in 10 min; Wave Length: 254/220 nm) to afford racemic product, which was further separated by PREP-CHIRAL_HPLC (Column: CHIRALPAK IE, 2*25 cm, 5 μm; Mobile Phase A: Hex:DCM=3:1 (0.5% 2M NH₃-MeOH)—HPLC, Mobile Phase B: EtOH—HPLC; Flow rate: 20 mL/min; Gradient: isocratic 30; Wave Length: 220/254 nm; RT1 (min): 11.866; RT2 (min): 15.685; Sample Solvent: EtOH—HPLC; Injection Volume: 0.6 mL) to give two products. The second eluting was lyophilized to afford (1R,3S)-3-{5-[(1S,2S)-2-(2-formyl-3-hydroxyphenyl)cyclopropaneamido]-2H-pyrazol-3-yl}cyclopentyl N-isopropylcarbamate (OAB, 5.7 mg, 2.97%) as a yellow solid. MS (ESI) mass calcd. for C₁₉H₂₂F₃NO, 440.20 m/z, found 441.25 [M+H]⁺. ¹H NMR (400 MHz, DMSO-d₆) δ 12.14-11.95 (m, 1H), 11.36 (s, 1H), 10.74-10.53 (m, 1H), 10.52-10.28 (m, 1H), 7.47 (t, J=8.0 Hz, 1H), 7.02-6.92 (m, 1H), 6.87 (d, J=8.4 Hz, 1H), 6.75 (d, J=7.6 Hz, 1H), 6.44-6.29 (m, 1H), 5.11-4.92 (m, 1H), 3.67-3.48 (m, 1H), 3.13-2.83 (m, 2H), 2.17-2.09 (m, 1H), 2.07-1.49 (m, 6H), 1.48-1.39 (m, 1H), 1.38-1.26 (m, 1H), 1.04 (d, J=6.6 Hz, 6H).

### Example 7 Representative Schemes Following General Scheme F

### Example 7.1: Synthesis of Compound 44: (1R,3S)-3-(3-(3-(4-formylpyridin-3-yl)-1-methyl-1H-pyrazole-5-carboxamido)-1H-pyrazol-5-yl)cyclopentyl isopropylcarbamate; trifluoroacetic acid

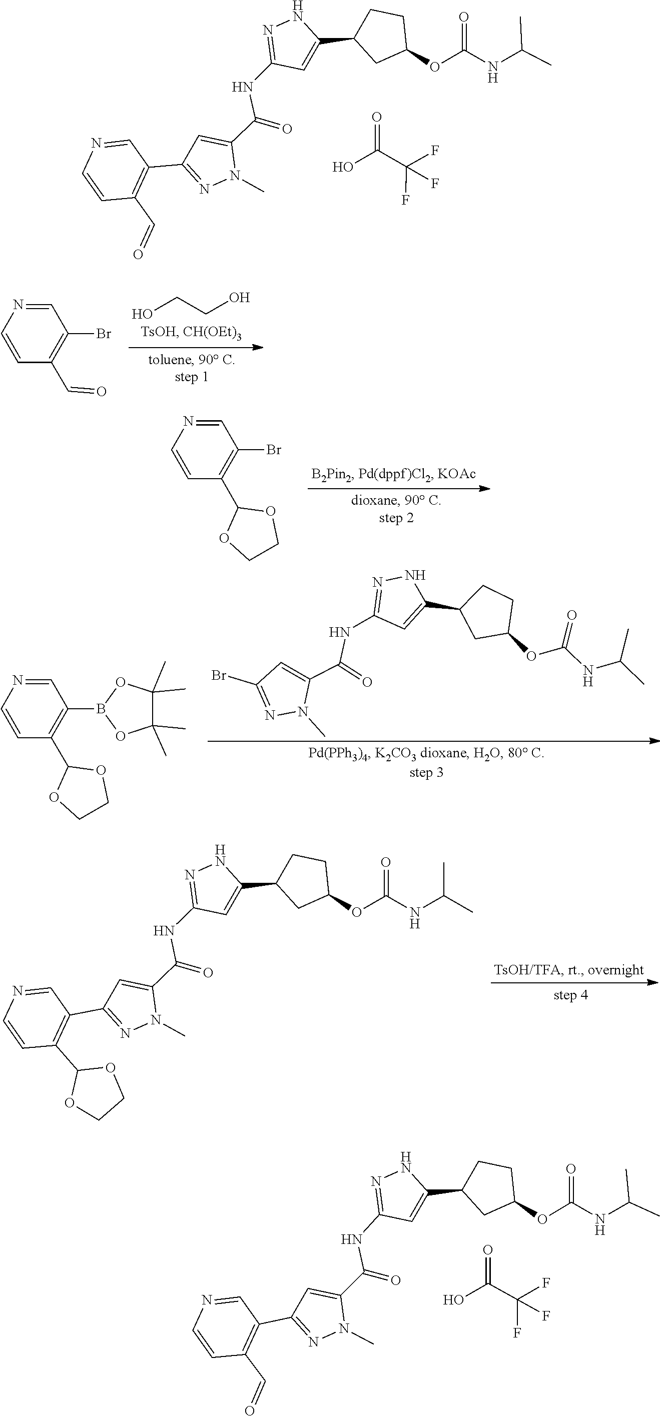

### Step 1: Synthesis of 3-bromo-4-(1,3-dioxolan-2-yl)pyridine

To a stirred solution of 3-bromopyridine-4-carbaldehyde (3 g, 16.128 mmol, 1 equiv) in toluene (60 mL, 563.918 mmol, 34.96 equiv) was added triethyl orthoformate (7.17 g, 48.384 mmol, 3 equiv), ethylene glycol (5.01 g, 80.640 mmol, 5 equiv) and TsOH (0.28 g, 1.613 mmol, 0.1 equiv) in portions at room temperature. The resulting mixture was stirred for 2 h at 90° C. The reaction mixture was then treated with H₂O (100 mL), dropwise over 10 min, extracted with EtOAc (100 mL×2), and the combined extracts washed with brine (100 mL), dried over anhydrous Na₂SO₄, filtered, and concentrated to dryness in vacuo to give a nearly yellow viscous oil. The oil was then subjected to silica gel chromatography (0-30% EtOAc/petroleum ether) to give 3-bromo-4-(1,3-dioxolan-2-yl)pyridine (2 g, 52.89%) as a light yellow solid. MS (ESI) calcd. for CH₈BrNO₂, 228.97 m/z, found 230.00 [M+H]⁺.

### Step 2: Synthesis of 4-(1,3-dioxolan-2-yl)-3-(4,4,5,5-tetramethyl-1,3,2-dioxaborolan-2-yl)pyridine

To a solution of 3-bromo-4-(1,3-dioxolan-2-yl)pyridine (500.0 mg, 2.173 mmol, 1 equiv) and bis(pinacolato)diboron (1655.7 mg, 6.519 mmol, 3 equiv) in dioxane (12.5 mL) were added Pd(dppf)Cl₂ (159.0 mg, 0.217 mmol, 0.1 equiv) and KOAc (533.2 mg, 5.433 mmol, 2.5 equiv). After stirring for 2 h at 90° C. under a nitrogen atmosphere. The reaction mixture was then treated with H₂O (50 mL), dropwise over 10 min, extracted with EtOAc (50 mL×2), and the combined extracts washed with brine (50 mL), dried over anhydrous Na₂SO₄, filtered, and concentrated to dryness in vacuo to give a nearly yellow viscous oil. The oil was then subjected to silica gel chromatography (0-30% EtOAc/petroleum ether) to give 4-(1,3-dioxolan-2-yl)-3-(4,4,5,5-tetramethyl-1,3,2-dioxaborolan-2-yl)pyridine (500.0 mg, 27.68%) as a brown solid. MS (ESI) calcd. for C₁₄H₂₀BNO₄, 277.15 m/z, found 278.20 [M+H]⁺.

### Step 3: Synthesis of (1R,3S)-3-(3-(3-(4-(1,3-dioxolan-2-yl)pyridin-3-yl)-1-methyl-1H-pyrazole-5-carboxamido)-1H-pyrazol-5-yl)cyclopentyl isopropylcarbamate

To a solution of 4-(1,3-dioxolan-2-yl)-3-(4,4,5,5-tetramethyl-1,3,2-dioxaborolan-2-yl)pyridine (492.0 mg, 1.776 mmol, 6 equiv) and (1R,3S)-3-[5-(5-bromo-2-methylpyrazole-3-amido)-2H-pyrazol-3-yl]cyclopentyl N-isopropylcarbamate (130.0 mg, 0.296 mmol, 1 equiv) in dioxane (4 mL) and H₂O (1 mL) were added Pd(PPh₃)₄ (34.20 mg, 0.030 mmol, 0.1 equiv) and K₂CO₃ (102.2 mg, 0.740 mmol, 2.5 equiv). After stirring for 2 h at 90° C. under a nitrogen atmosphere. The reaction mixture was then treated with H₂O (10 mL), dropwise over 10 min, extracted with EtOAc (10 mL×2), and the combined extracts washed with brine (10 mL), dried over anhydrous Na₂SO₄. After filtration, the filtrate was concentrated under reduced pressure. This resulted in (1R,3S)-3-(3-(3-(4-(1,3-dioxolan-2-yl)pyridin-3-yl)-1-methyl-1H-pyrazole-5-carboxamido)-1H-pyrazol-5-yl)cyclopentyl isopropylcarbamate (37.0 mg, 24.54%) as a brown solid. MS (ESI) calcd. for C₂₅H₃₁N₇O₅, 509.24 m/z, found 510.25 [M+H]⁺.

### Step 4: Synthesis of (1R,3S)-3-(3-(3-(4-formylpyridin-3-yl)-1-methyl-1H-pyrazole-5-carboxamido)-1H-pyrazol-5-yl)cyclopentyl isopropylcarbamate; trifluoroacetic acid

To a stirred solution of (1R,3S)-3-(5-{5-[4-(1,3-dioxolan-2-yl)pyridin-3-yl]-2-methylpyrazole-3-amido}-2H-pyrazol-3-yl)cyclopentyl N-isopropylcarbamate (30.0 mg, 0.059 mmol, 1 equiv) in TFA (0.3 mL) was added TsOH (0.1 mL) dropwise at room temperature. The resulting mixture was stirred for overnight at room temperature. The mixture was basified to pH ˜8 with Na₂CO₃. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, NH₄Cl (0.05 mmol/L) in ACN, 5% to 60% gradient in 20 min; detector, UV 254 nm. This resulted in (1R,3S)-3-{5-[5-(4-formylpyridin-3-yl)-2-methylpyrazole-3-amido]-2H-pyrazol-3-yl}cyclopentyl N-isopropylcarbamate (1.8 mg, 6.10%) as a white solid. MS (ESI) calcd. for C₂₅H₂₈F₃N₇O₆, 465.21 m/z, found 466.25 [M+H]⁺. ¹H NMR (400 MHz, DMSO-d₆+D₂O) δ 10.45 (s, 1H), 9.05 (s, 1H), 8.79-8.80 (m, 1H), 7.49-7.88 (m, 2H), 6.22-6.47 (m, 1H), 4.98-5.03 (m, 1H), 4.19 (s, 3H), 3.55-3.56 (m, 1H), 3.08-3.13 (m, 1H), 2.45-2.47 (m, 1H), 2.03-2.08 (m, 1H), 1.86-1.95 (m, 1H), 1.74-1.76 (m, 2H), 1.62-1.66 (m, 1H), 1.03-1.04 (m, 6H). ¹⁹F NMR (376 MHz, DMSO-d₆) δ −73.77.

### Example 7.2: Synthesis of Compound 31: (1R,3S)-3-{5-[5-(3-formyl-4-hydroxyphenyl)-2-methylpyrazole-3-amido]-2H-pyrazol-3-yl}cyclopentyl N-isopropylcarbamate

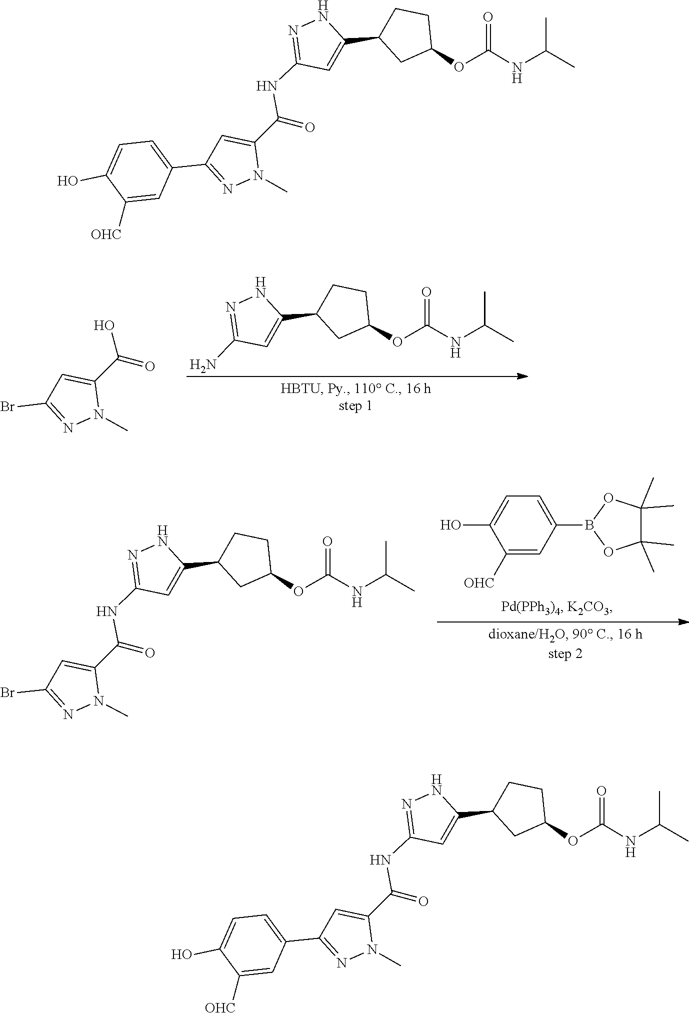

### Step 1: Synthesis of (1R,3S)-3-[5-(5-bromo-2-methylpyrazole-3-amido)-2H-pyrazol-3-yl]cyclopentyl N-isopropylcarbamate

To a stirred solution of 5-bromo-2-methylpyrazole-3-carboxylic acid (325.0 mg, 1.586 mmol, 2 equiv) and (1R,3S)-3-(5-amino-2H-pyrazol-3-yl)cyclopentyl N-isopropylcarbamate (200.0 mg, 0.793 mmol, 1.00 equiv) in pyridine (10 mL) was added HBTU (360.7 mg, 0.952 mmol, 1.2 equiv) at 25° C. Then the resulting mixture was stirred for additional 16 h at 110° C. The mixture was diluted with ethyl acetate (20 mL), washed with water (20 mL×3) and brine (10 mL), dried over anhydrous sodium sulfate, and concentrated in vacuum. The residue was purified by silica gel column chromatography, eluted with MeOH/CH₂Cl₂ (0-5%) to afford (1R,3S)-3-[5-(5-bromo-2-methylpyrazole-3-amido)-2H-pyrazol-3-yl]cyclopentyl N-isopropylcarbamate (317.0 mg, 79.04%) as a brown yellow oil. MS (ESI) calcd. for C₁₇H₂₃BrN₆O₃, 438.10 m/z, found 439.05 [M+H]⁺.

### Step 2: Synthesis of (1R,3S)-3-{5-[5-(3-formyl-4-hydroxyphenyl)-2-methylpyrazole-3-amido]-2H-pyrazol-3-yl}cyclopentyl N-isopropylcarbamate

To a stirred solution of (1R,3S)-3-[5-(5-bromo-2-methylpyrazole-3-amido)-2H-pyrazol-3-yl]cyclopentyl N-isopropylcarbamate (150.0 mg, 0.341 mmol, 1 equiv) and 2-hydroxy-5-(4,4,5,5-tetramethyl-1,3,2-dioxaborolan-2-yl)benzaldehyde (127.1 mg, 0.512 mmol, 1.5 equiv) in 1,4-dioxane (2.5 mL)/H₂O (0.5 mL) were added Pd(PPh₃)₄ (39.5 mg, 0.034 mmol, 0.1 equiv) and K₂CO₃ (117.9 mg, 0.853 mmol, 2.5 equiv) at 25° C. under nitrogen atmosphere. Then the resulting mixture was stirred for additional 16 h for 90° C. The mixture was diluted with ethyl acetate (70 mL), washed with water (20 mL×3) and brine (40 mL), dried over anhydrous sodium sulfate, and concentrated in vacuum. Then the crude product was further purified by Prep-HPLC with the following condition: Column: XSelect CSH 5 m, 19 mm×150 mm; Mobile Phase A: Water (0.1% FA), Mobile Phase B: ACN; Flow rate: 25 mL/min mL/min; Gradient: 33% B to 63% B in 7 min; Wave Length: 254/220 nm; RT1 (min): 6.38, it was afforded (1R,3S)-3-{5-[5-(3-formyl-4-hydroxyphenyl)-2-methylpyrazole-3-amido]-2H-pyrazol-3-yl}cyclopentyl N-isopropylcarbamate (16.4 mg, 9.8%) as a white solid. MS (ESI) calcd. for C₂₄H₂₈N₆O₅, 480.21 m/z, found 481.20 [M+H]⁺. ¹H NMR (300 MHz, DMSO-d₆) δ 12.34 (s, 1H), 11.86 (s, 1H), 10.95 (s, 1H), 10.38 (s, 1H), 7.67-7.79 (m, 1H), 7.50 (s, 1H), 7.15-7.25 (m, 1H), 6.85-7.00 (m, 2H), 6.47 (s, 1H), 5.01 (s, 1H), 4.17 (s, 3H), 3.59-3.70 (m, 1H), 3.04-3.15 (m, 1H), 2.32 (s, 1H), 1.98-2.11 (m, 1H), 1.90 (s, 1H), 1.75 (d, J=8.4 Hz, 2H), 1.60-1.70 (m, 1H), 1.00-1.10 (m, 6H).

### Example 7.3: Synthesis of Compound 41: (1R,3S)-3-{5-[5-(2-formyl-3-methanesulfonamido phenyl)-2-methylpyrazole-3-amido]-2H-pyrazol-3-yl}cyclopentyl N-isopropylcarbamate; trifluoroacetic acid

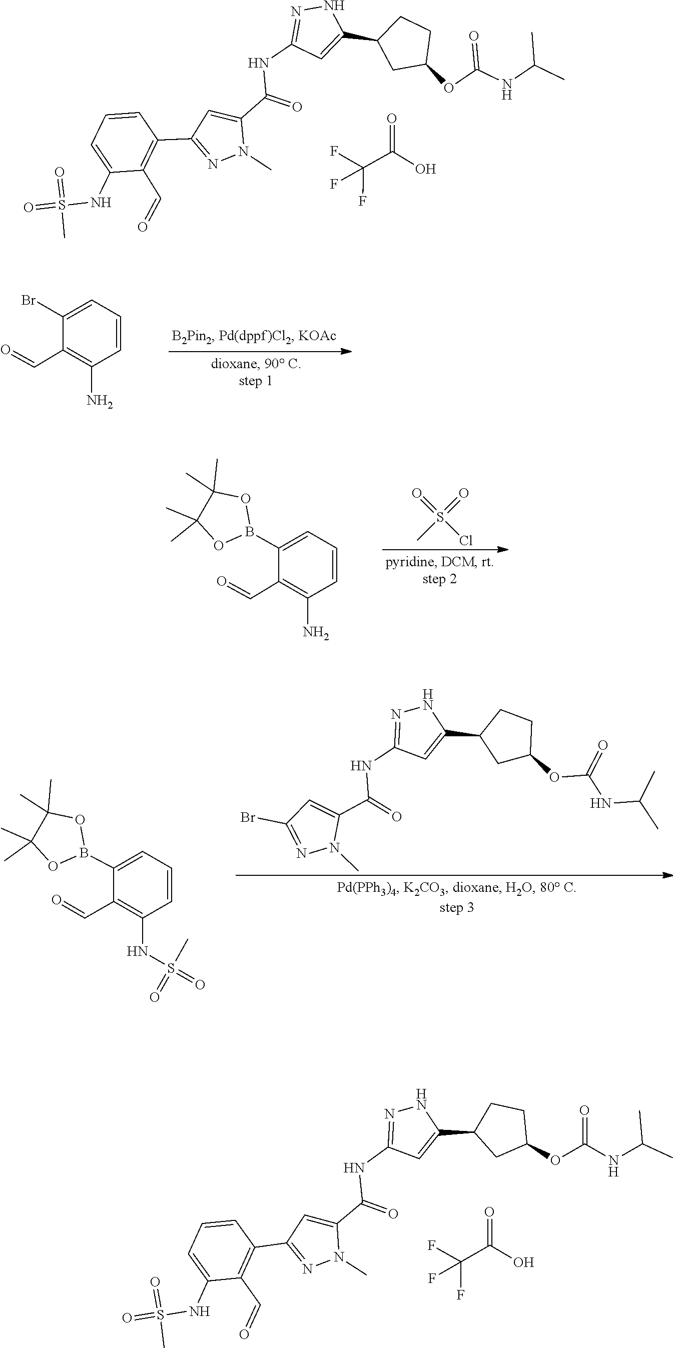

### Step 1: Synthesis of 2-amino-6-(4,4,5,5-tetramethyl-1,3,2-dioxaborolan-2-yl)benzaldehyde

To a stirred mixture of 2-amino-6-bromobenzaldehyde (1.5 g, 7.499 mmol, 1 equiv) and bis(pinacolato)diboron (7.62 g, 29.996 mmol, 4 equiv) in dioxane (100 mL, 1180.396 mmol) were added Pd(dppf)Cl₂ (1.10 g, 1.500 mmol, 0.2 equiv) and AcOK (1.84 g, 18.747 mmol, 2.5 equiv) at room temperature under N₂ atmosphere. The resulting mixture was stirred for 2 h at 80° C. under N₂ atmosphere then cooled to room temperature and diluted with water. The resulting mixture was extracted with EA (3×300 mL). The combined organic layers were washed with brine (3×300 mL), dried over anhydrous Na₂SO₄. After filtration, the filtrate was concentrated under reduced pressure. The resulting mixture was concentrated under reduced pressure. The residue was purified by silica gel column chromatography, eluted with PE/EA (20%) to afford 2-amino-6-(4,4,5,5-tetramethyl-1,3,2-dioxaborolan-2-yl)benzaldehyde (0.79 g, 42.64%) as a yellow solid. MS (ESI) calcd. for C₁₃H₁₈BNO₃, 247.14 m/z, found 248.15 [M+H]⁺.

### Step 2: Synthesis of N-[2-formyl-3-(4,4,5,5-tetramethyl-1,3,2-dioxaborolan-2-yl)phenyl]methanesulfonamide

To a stirred solution of 2-amino-6-(4,4,5,5-tetramethyl-1,3,2-dioxaborolan-2-yl)benzaldehyde (0.7 g, 2.833 mmol, 1 equiv) in DCM (10 mL) was added pyridine (2.24 g, 28.319 mmol, 10.00 equiv) and methanesulfonyl chloride (3.24 g, 28.330 mmol, 10 equiv) dropwise at 0° C. under N₂ atmosphere. The resulting mixture was stirred for 6 h at room temperature under N₂ atmosphere. The reaction was quenched by the addition of H₂O (10 mL) at room temperature. The resulting mixture was extracted with DCM (3×30 mL). The combined organic layers were washed with brine (3×30 mL). After filtration, the filtrate was concentrated in vacuo. The residue was purified by silica gel column chromatography, eluted with 10% PE/EA to afford N-[2-formyl-3-(4,4,5,5-tetramethyl-1,3,2-dioxaborolan-2-yl)phenyl]methanesulfonamide (0.3 g, 32.57%) as a yellow solid. MS (ESI) calcd. for C₁₄H₂₀BNO₅S, 325.12 m/z, found 324.10 [M−H]⁻.

### Step 3: Synthesis of (1R,3S)-3-{5-[5-(2-formyl-3-methanesulfonamidophenyl)-2-methyl pyrazole-3-amido]-2H-pyrazol-3-yl}cyclopentyl N-isopropylcarbamate; trifluoroacetic acid

To a stirred mixture of N-[2-formyl-3-(4,4,5,5-tetramethyl-1,3,2-dioxaborolan-2-yl)phenyl]methanesulfonamide (384.9 mg, 1.184 mmol, 4 equiv) and (1R,3S)-3-[5-(5-bromo-2-methylpyrazole-3-amido)-2H-pyrazol-3-yl]cyclopentyl N-isopropylcarbamate (130.0 mg, 0.296 mmol, 1.00 equiv) in dioxane (10 mL, 118.040 mmol) and H₂O (2 mL, 0.056 mmol) were added Pd(PPh₃)₄ (68.4 mg, 0.059 mmol, 0.2 equiv) and K₂CO₃ (102.2 mg, 0.740 mmol, 2.5 equiv) at room temperature under N₂ atmosphere. The resulting mixture was stirred for 1 h at 80° C. under N₂ atmosphere, then cooled to room temperature and diluted with water. The resulting mixture was extracted with EA (3×30 mL). The combined organic layers were washed with brine (3×30 mL), dried over anhydrous Na₂SO₄. After filtration, the filtrate was concentrated under reduced pressure. The crude product was purified by Prep-HPLC with the following conditions (Column: Xselect CSH Prep C18 OBD Column, 30×150 mm, 5 um; Mobile Phase A: Water (0.05% TFA), Mobile Phase B: ACN; Flow rate: 60 mL/min; Gradient: 31% B to 53% B in 10 min; Wave Length: 254 nm/220 nm nm) to afford (1R,3S)-3-{5-[5-(2-formyl-3-methanesulfonamidophenyl)-2-methylpyrazole-3-amido]-2H-pyrazol-3-yl}cyclopentyl N-isopropylcarbamate; trifluoroacetic acid (10.0 mg, 4.90%) as a white solid. MS (ESI) calcd. for C₂₇H₃₂F₃N₇O₈S, 557.21 m/z, found 558.15 [M+H]⁺. ¹H NMR (400 MHz, DMSO-d₆) δ (ppm): 10.97 (s, 1H), 10.85 (s, 1H), 10.22 (s, 1H), 7.76-7.80 (m, 1H), 7.61-7.63 (m, 1H), 7.50 (s, 1H), 7.40-7.42 (m, 1H), 6.96-6.98 (m, 1H), 6.46 (s, 1H), 5.01-5.02 (m, 1H), 4.17 (s, 3H), 3.56-3.61 (m, 1H), 3.28 (s, 3H), 3.07-3.11 (m, 1H), 2.51-2.52 (m, 1H), 2.02-2.08 (m, 1H), 1.87-1.93 (m, 1H), 1.70-1.76 (m, 2H), 1.59-1.66 (m, 1H), 1.03-1.05 (m, 6H). ¹⁹F NMR (376 MHz, DMSO-d₆) δ (ppm): −74.72.

### Example 7.4: Synthesis of Compound 42: (1R,3S)-3-{5-[5-(5-fluoro-2-formyl-3-hydroxyphenyl)-2-methylpyrazole-3-amido]-2H-pyrazol-3-yl}cyclopentyl N-isopropylcarbamate; trifluoroacetic acid

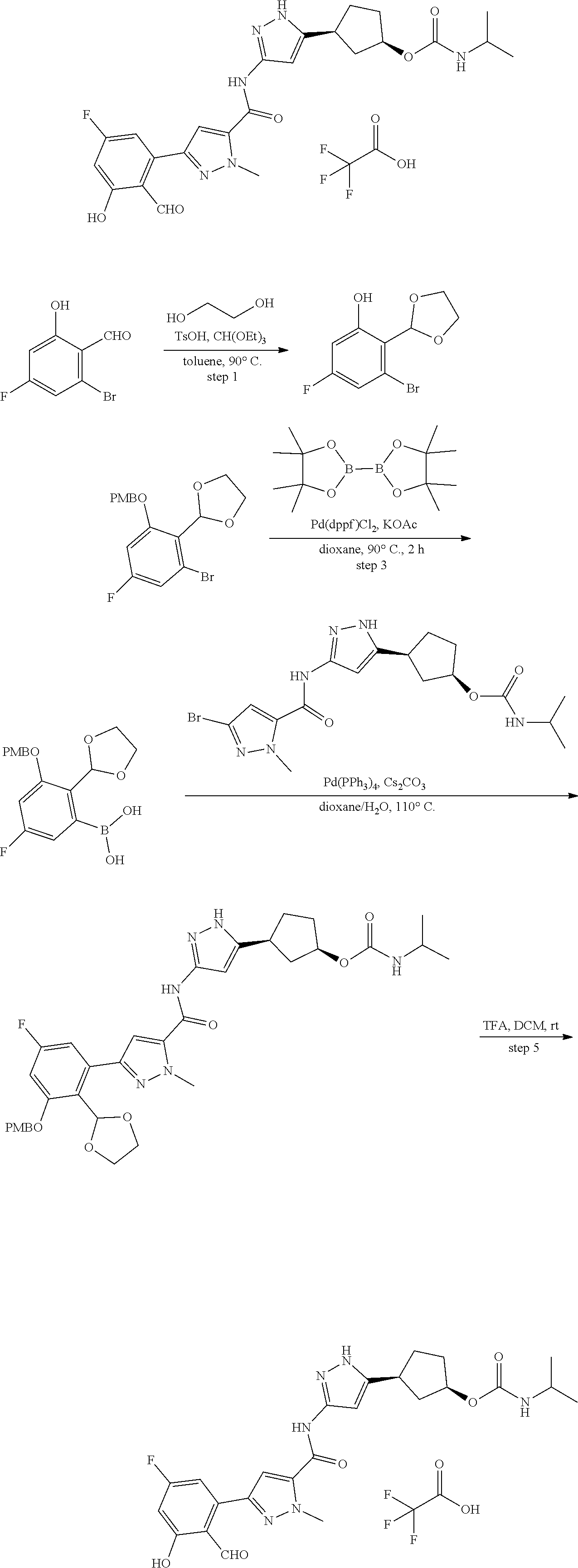

### Step 1: Synthesis of 3-bromo-2-(1,3-dioxolan-2-yl)-5-fluorophenol

To a stirred solution of 2-bromo-4-fluoro-6-hydroxybenzaldehyde (600.0 mg, 2.740 mmol, 1 equiv) in anhydrous toluene (20 mL) was added triethyl orthoformate (1218.0 mg, 8.220 mmol, 3 equiv) and ethylene glycol (850.2 mg, 13.700 mmol, 5 equiv) followed by catalytic amount of TsOH (47.2 mg, 0.274 mmol, 0.1 equiv) at room temperature. The reaction mixture was stirred at 90° C. for a period of 8 h. Desired product could be detected by LCMS. The resulting mixture was extracted with EA (3×50 mL). The combined organic layers were washed with brine (3×50 mL), dried over anhydrous Na₂SO₄. After filtration, the filtrate was concentrated under reduced pressure. The residue was purified by silica gel column chromatography (0-30% EtOAc/PE) to afford 3-bromo-2-(1,3-dioxolan-2-yl)-5-fluorophenol (600.0 mg, 83.25%) as a yellow solid. MS (ESI) calcd. for C₉H₈BrFO₃, 261.96 m/z, found 263.20 [M+H]⁺.

### Step 2: Synthesis of 2-(2-bromo-4-fluoro-6-((4-methoxybenzyl)oxy)phenyl)-1,3-dioxolane

To a stirred solution of 3-bromo-2-(1,3-dioxolan-2-yl)-5-fluorophenol (550.0 mg, 2.091 mmol, 1 equiv), K₂CO₃ (1011.3 mg, 7.319 mmol, 3.5 equiv), KI (416.5 mg, 2.509 mmol, 1.2 equiv) in anhydrous DMF (20 mL) was added 4-methoxybenzyl chloride (392.9 mg, 2.509 mmol, 1.2 equiv) at 70° C. and stirred for 16 h, then cooled to room temperature and diluted with water. The mixture was extracted with EA (3×50 mL). The combined organic layers were washed with brine (3×20 mL), dried over anhydrous Na₂SO₄. After filtration, the filtrate was concentrated under reduced pressure. The residue was purified by silica gel column chromatography (0-40% EA/PE) to afford 2-{2-bromo-4-fluoro-6-[(4-methoxyphenyl) methoxy]phenyl}-1,3-dioxolane (500.0 mg, 62.41%) as a yellow oil. MS (ESI) calcd. for C₁₇H₁₆BrFO₄, 382.02 m/z, found 383.10 [M+H]⁺.

### Step 3: Synthesis of 2-(2-(1,3-dioxolan-2-yl)-5-fluoro-3-((4-methoxybenzyl)oxy)phenyl)-4,4,5,5-tetramethyl-1,3,2-dioxaborolane

To a stirred solution of 2-{2-bromo-4-fluoro-6-[(4-methoxyphenyl)methoxy]phenyl}-1,3-dioxolane (450.0 mg, 1.174 mmol, 1 equiv) in anhydrous dioxane (10 mL) was added bis(pinacolato)diboron (1192.8 mg, 4.696 mmol, 4 equiv) and KOAc (288.1 mg, 2.935 mmol, 2.5 equiv) followed by catalytic amount of Pd(dppf)Cl₂ (128.9 mg, 0.176 mmol, 0.15 equiv) at room temperature. The reaction mixture was stirred at 90° C. for a period of 2 h under nitrogen atmosphere. Desired product could be detected by LCMS. After completion of reaction, the resulting mixture was extracted with EA (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with EA:PE (1:2) to afford 2-[2-(1,3-dioxolan-2-yl)-5-fluoro-3-[(4-methoxyphenyl)methoxy]phenyl]-4,4,5,5-tetramethyl-1,3,2-dioxaborolane (300.0 mg, 59.37%) as a white oil. MS (ESI) calcd. for C₂₃H₂₈BFO₆, 348.12 m/z, found 347.12 [M−H]⁻.

### Step 4: Synthesis of (1R,3S)-3-(3-(3-(2-(1,3-dioxolan-2-yl)-5-fluoro-3-((4-methoxybenzyl) oxy)phenyl)-1-methyl-1H-pyrazole-5-carboxamido)-1H-pyrazol-5-yl)cyclopentyl isopropylcarbamate

To a solution of 2-[2-(1,3-dioxolan-2-yl)-5-fluoro-3-[(4-methoxyphenyl)methoxy]phenyl]-4,4,5,5-tetramethyl-1,3,2-dioxaborolane (222.0 mg, 0.516 mmol, 2.8 equiv) and (1R,3S)-3-[5-(5-bromo-2-methylpyrazole-3-amido)-2H-pyrazol-3-yl]cyclopentyl N-isopropylcarbamate (90.0 mg, 0.184 mmol, 1 equiv) were added Cs₂CO₃ (150.1 mg, 0.461 mmol, 2.5 equiv) and Pd(PPh₃)₄ (21.3 mg, 0.018 mmol, 0.1 equiv) in dioxane (5 mL) and H₂O (1 mL). After stirring for 1 h at 110° C. under nitrogen atmosphere, the resulting mixture was cooled to room temperature, diluted with water and extracted with EA (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with EA:PE (1:3) to afford (1R,3S)-3-(5-{5-[2-(1,3-dioxolan-2-yl)-5-fluoro-3-[(4-methoxyphenyl)methoxy]phenyl]-2-methylpyrazole-3-amido}-2H-pyrazol-3-yl)cyclopentyl N-isopropylcarbamate (82.0 mg, 67.15%) as a yellow solid. MS (ESI) calcd. for C₃₄H₃₉FN₆O₇, 662.29 m/z, found 663.35 [M+H]⁺.

### Step 5: Synthesis of (1R,3S)-3-{5-[5-(5-fluoro-2-formyl-3-hydroxyphenyl)-2-methylpyrazole-3-amido]-2H-pyrazol-3-yl}cyclopentyl N-isopropylcarbamate; trifluoroacetic acid

To a stirred solution of (1R,3S)-3-(5-{5-[2-(1,3-dioxolan-2-yl)-5-fluoro-3-[(4-methoxyphenyl) methoxy]phenyl]-2-methylpyrazole-3-amido}-2H-pyrazol-3-yl)cyclopentyl N-isopropyl carbamate (80.0 mg, 0.121 mmol, 1 equiv) in DCM (3 mL) were added TFA (1 mL) dropwise at room temperature. The resulting mixture was stirred for 0.5 h. The reaction was monitored by LCMS. The resulting mixture was concentrated under reduced pressure. The crude product was purified by Prep-HPLC with the following conditions (Column: XBridge Shield RP18 OBD Column, 30×150 mm, 5 um; Mobile Phase A: Water (0.05% TFA), Mobile Phase B: ACN; Flow rate: 60 mL/min; Gradient: 37% B to 59% B in 10 min; Wave Length: 254/220 nm; RT1 (min): 10) to afford (1R,3S)-3-{5-[5-(5-fluoro-2-formyl-3-hydroxyphenyl)-2-methylpyrazole-3-amido]-2H-pyrazol-3-yl}cyclopentyl N-isopropylcarbamate; trifluoroacetic acid (9.4 mg, 12.59%) as a white solid. MS(ESI) calcd for C₂₄H₂₇FN₆O₅, 498.20 m/z, found 499.25 [M+H]⁺. ¹H NMR (400 MHz, DMSO-d₆) δ 12.27 (br s, 1H), 10.96 (br s, 1H), 10.34 (s, 1H), 7.53 (br s, 1H), 6.91-7.02 (m, 3H), 6.47 (s, 1H), 4.99-5.01 (m, 1H), 4.18 (s, 3H), 3.55-3.63 (m, 1H), 3.05-3.11 (m, 1H), 2.46-2.47 (m, 1H), 2.02-2.08 (m, 1H), 1.91-1.94 (m, 1H), 1.63-1.78 (m, 3H), 1.03-1.05 (d, J=8.0 Hz, 6H). ¹⁹F NMR (376 MHz, DMSO-d₆) δ −74.76, −99.24.

### Example 7.5: Synthesis of Compound 48: (1R,3S)-3-{5-[5-(2-formyl-3-hydroxy-5-methoxy phenyl)-2-methylpyrazole-3-amido]-2H-pyrazol-3-yl}cyclopentyl N-isopropylcarbamate; trifluoroacetic acid

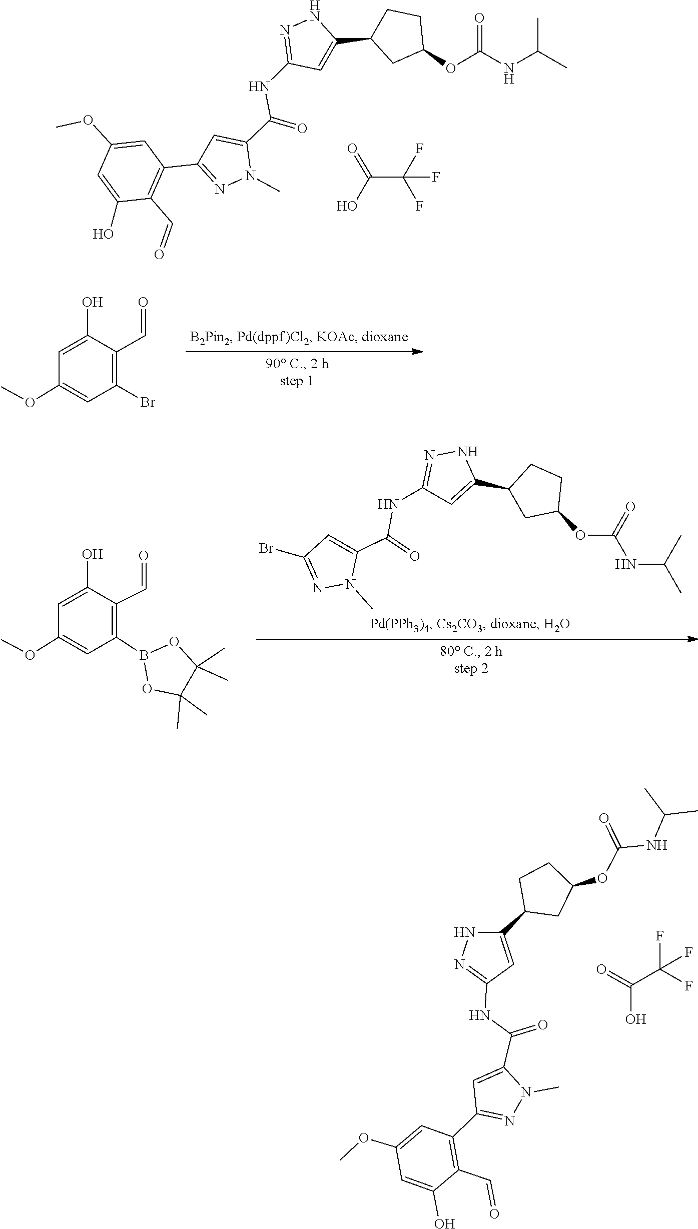

### Step 1: Synthesis of 2-hydroxy-4-methoxy-6-(4,4,5,5-tetramethyl-1,3,2-dioxaborolan-2-yl)benzaldehyde

To a stirred solution of 2-bromo-6-hydroxy-4-methoxybenzaldehyde (500.0 mg, 2.164 mmol, 1 equiv) and 4,4,5,5-tetramethyl-2-(4,4,5,5-tetramethyl-1,3,2-dioxaborolan-2-yl)-1,3,2-dioxaborolane (2.20 g, 8.656 mmol, 4 equiv) in dioxane (10 mL) were added KOAc (531.0 mg, 5.410 mmol, 2.5 equiv) and Pd(dppf)Cl₂ (158.4 mg, 0.216 mmol, 0.10 equiv) in portions at room temperature under nitrogen atmosphere. The mixture was stirred at 90° C. for 2 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (8:1) to afford 2-hydroxy-4-methoxy-6-(4,4,5,5-tetramethyl-1,3,2-dioxaborolan-2-yl)benzaldehyde (600.0 mg, 69.86%) as a yellow solid. MS(ESI) calcd. for C₁₄H₁₉BO₅: 278.13 m/z, found: 279.05 [M+H]⁺.

### Step 2: Synthesis of (1R,3S)-3-{5-[5-(2-formyl-3-hydroxy-5-methoxyphenyl)-2-methyl pyrazole-3-amido]-2H-pyrazol-3-yl}cyclopentyl N-isopropylcarbamate; trifluoroacetic acid

To a stirred solution of 2-hydroxy-4-methoxy-6-(4,4,5,5-tetramethyl-1,3,2-dioxaborolan-2-yl)benzaldehyde (189.9 mg, 0.682 mmol, 2 equiv) and (1R,3S)-3-[5-(5-bromo-2-methylpyrazole-3-amido)-2H-pyrazol-3-yl]cyclopentyl N-isopropylcarbamate (150.0 mg, 0.341 mmol, 1.00 equiv) in dioxane (5 mL) H₂O (1 mL) were added K₂CO₃ (118.0 mg, 0.853 mmol, 2.5 equiv) and Pd(PPh₃)₄ (39.5 mg, 0.034 mmol, 0.1 equiv) in portions at room temperature under nitrogen atmosphere. The mixture was stirred at 80° C. for 2 h. The reaction was quenched with water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (TFA), 50% to 70% gradient in 20 min; detector, UV 254 nm. The crude product (100.0 mg) was purified by Prep-HPLC with the following conditions (Column: Xselect CSH OBD Column, 30×150 mm, 5 um; Mobile Phase A: Water (0.05% TFA), Mobile Phase B: ACN; Flow rate: 60 mL/min; Gradient: 29% B to 51% B in 11 min; Wave Length: 254 nm/220 nm; RT1 (min): 11) to afford (1R,3S)-3-{5-[5-(2-formyl-3-hydroxy-5-methoxyphenyl)-2-methylpyrazole-3-amido]-2H-pyrazol-3-yl}cyclopentyl N-isopropylcarbamate; trifluoroacetic acid (5.2 mg, 2.42%) as an off-white solid. MS (ESI) calcd. for C₂₅H₃₀N₆O₆, 510.22 m/z, found 511.25 [M+H]⁺. ¹H NMR (400 MHz, DMSO-d₆) δ 12.56 (s, 1H), 10.92 (s, 1H), 10.25 (s, 1H), 7.49 (s, 1H), 6.95-7.09 (m, 1H), 6.72 (d, J=2.4 Hz, 1H), 6.56 (d, J=2.4 Hz, 1H), 6.47 (s, 1H), 4.97-5.05 (m, 1H), 4.15 (s, 3H), 3.92 (s, 3H), 3.57-3.62 (m, 1H), 3.07-3.12 (m, 1H), 2.47-2.49 (m, 1H), 2.02-2.08 (m, 1H), 1.88-1.94 (m, 1H), 1.74-1.77 (m, 2H), 1.57-1.68 (m, 1H), 1.03-1.05 (m, 6H). ¹⁹F NMR (376 MHz, DMSO-d₆) δ −74.55.

### Example 7.6: Synthesis of Compound 50: (1R,3S)-3-{5-[5-(2-formyl-3-hydroxyphenyl)-2-methyl pyrazole-3-amido]-2H-pyrazol-3-yl}cyclopentyl N-methylcarbamate; trifluoroacetic acid

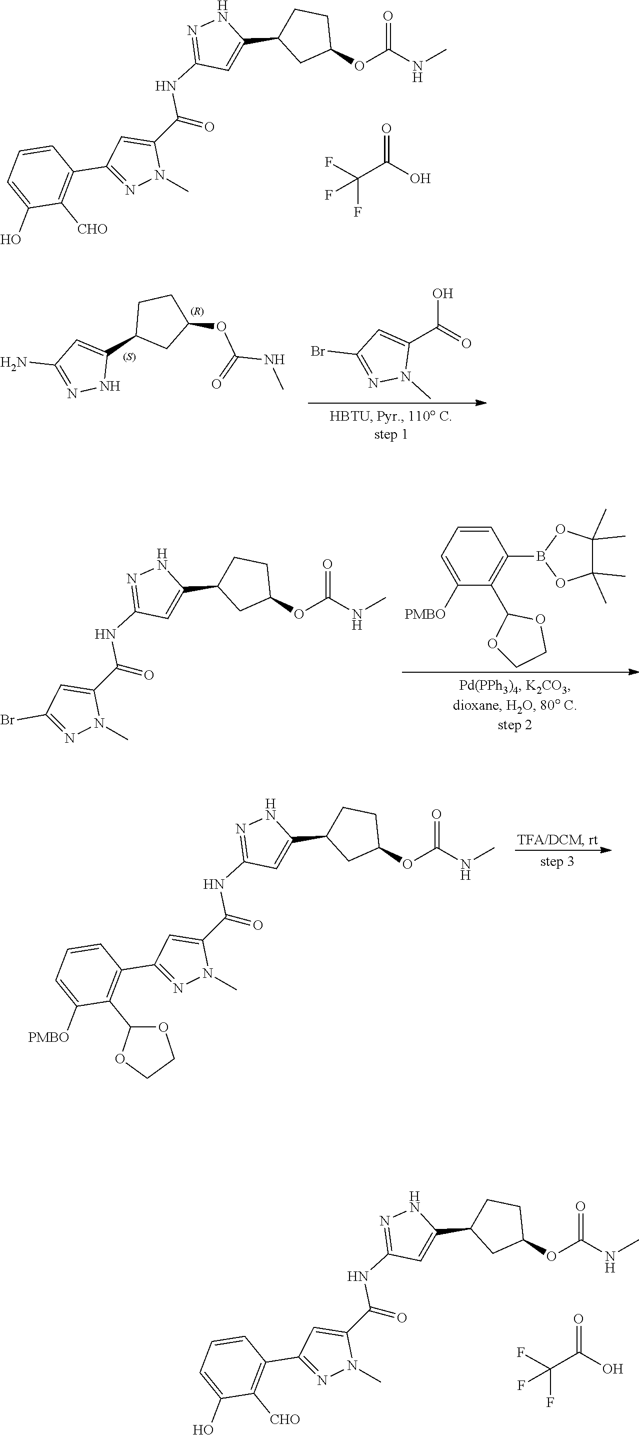

### Step 1: Synthesis of (1R,3S)-3-[5-(5-bromo-2-methylpyrazole-3-amido)-2H-pyrazol-3-yl]cyclopentyl N-methylcarbamate

To a stirred solution of (1R,3S)-3-(5-amino-2H-pyrazol-3-yl)cyclopentyl N-methylcarbamate (200.0 mg, 0.892 mmol, 1 equiv) and 5-bromo-2-methylpyrazole-3-carboxylic acid (182.8 mg, 0.892 mmol, 1 equiv) in pyridine (5 mL) were added HBTU (405.8 mg, 1.070 mmol, 1.2 equiv) at room temperature. The resulting mixture was stirred for 2 h at 110° C. The resulting mixture was diluted with water and extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with EA:PE (5:1) to afford (1R,3S)-3-[5-(5-bromo-2-methylpyrazole-3-amido)-2H-pyrazol-3-yl]cyclopentyl N-methylcarbamate (350.0 mg, 95.43%) as yellow oil. MS (ESI) calcd. for C₁₅H₁₉BrN₆O₃, 410.07 m/z, found 413.00 [M+H]⁺.

### Step 2: Synthesis of (1R,3S)-3-(5-{5-[2-(1,3-dioxolan-2-yl)-3-[(4-methoxyphenyl)methoxy]phenyl]-2-methylpyrazole-3-amido}-2H-pyrazol-3-yl)cyclopentyl N-methylcarbamate

To a stirred solution of (1R,3S)-3-[5-(5-bromo-2-methylpyrazole-3-amido)-2H-pyrazol-3-yl]cyclopentyl N-methylcarbamate (130.0 mg, 0.316 mmol, 1 equiv) in dioxane (3 mL) and H₂O (0.5 mL) was added tetrakis(triphenylphosphine)palladium(0) (54.8 mg, 0.047 mmol, 0.15 equiv) and K₂CO₃ (109.2 mg, 0.790 mmol, 2.5 equiv) at room temperature under N₂ atmosphere. The resulting mixture was stirred for 4 h at 90° C. under N₂ atmosphere. The resulting mixture was diluted with water and extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with EA:PE (10:1) to afford (1R,3S)-3-(5-{5-[2-(1,3-dioxolan-2-yl)-3-[(4-methoxyphenyl)methoxy]phenyl]-2-methylpyrazole-3-amido}-2H-pyrazol-3-yl)cyclopentyl N-methylcarbamate (70.0 mg, 35.91%) as a yellow solid. MS (ESI) calcd. for C₃₂H₃₆N₆O₇, 616.26 m/z, found 617.20 [M+H]⁺.

### Step 3: Synthesis of (1R,3S)-3-{5-[5-(2-formyl-3-hydroxyphenyl)-2-methylpyrazole-3-amido]-2H-pyrazol-3-yl}cyclopentyl N-methylcarbamate; trifluoroacetic acid

A solution of (1R,3S)-3-(5-{5-[2-(1,3-dioxolan-2-yl)-3-[(4-methoxyphenyl)methoxy]phenyl]-2-methylpyrazole-3-amido}-2H-pyrazol-3-yl)cyclopentyl N-methylcarbamate (70.0 mg, 0.114 mmol, 1 equiv) in DCM (1.5 mL) and TFA (0.3 mL) was stirred for 2 h at room temperature. The resulting mixture was concentrated under vacuum. The crude product was purified by Prep-HPLC with the following conditions (Column: Xselect CSH C18 OBD Column, 30×150 mm, 5 μm; Mobile Phase A: Water (0.05% TFA), Mobile Phase B: ACN; Flow rate: 60 mL/min; Gradient: 27% B to 57% B in 7 min; Wave Length: 254 nm/220 nm) to afford (1R,3S)-3-{5-[5-(2-formyl-3-hydroxyphenyl)-2-methylpyrazole-3-amido]-2H-pyrazol-3-yl}cyclopentyl N-methylcarbamate; trifluoroacetic acid (3.2 mg, 4.97%) as a white solid. MS (ESI) calcd. for C₂₂H₂₄N₆O₅, 452.18 m/z, found 453.20 [M+H]⁺. ¹H NMR (400 MHz, DMSO-d₆) δ 11.86 (br s, 1H), 10.94 (s, 1H), 10.38 (s, 1H), 7.65-7.69 (m, 1H), 7.49 (s, 1H), 7.17-7.19 (m, 1H), 7.01-7.03 (m, 1H), 6.93-6.97 (m, 1H), 6.45-6.46 (m, 1H), 5.02-5.03 (m, 1H), 4.18 (s, 3H), 3.06-3.14 (m, 1H), 2.56-2.57 (m, 3H), 2.44-2.47 (m, 1H), 2.02-2.08 (m, 1H), 1.86-1.96 (m, 1H), 1.67-1.78 (m, 2H), 1.60-1.67 (m, 1H). ¹⁹F NMR (376 MHz, DMSO-d₆) δ −74.67.

### Example 7.7: Synthesis of Compound 52: (1R,3S)-3-(3-(3-(3-ethynyl-2-formylphenyl)-1-methyl-1H-pyrazole-5-carboxamido)-1H-pyrazol-5-yl)cyclopentyl isopropylcarbamate

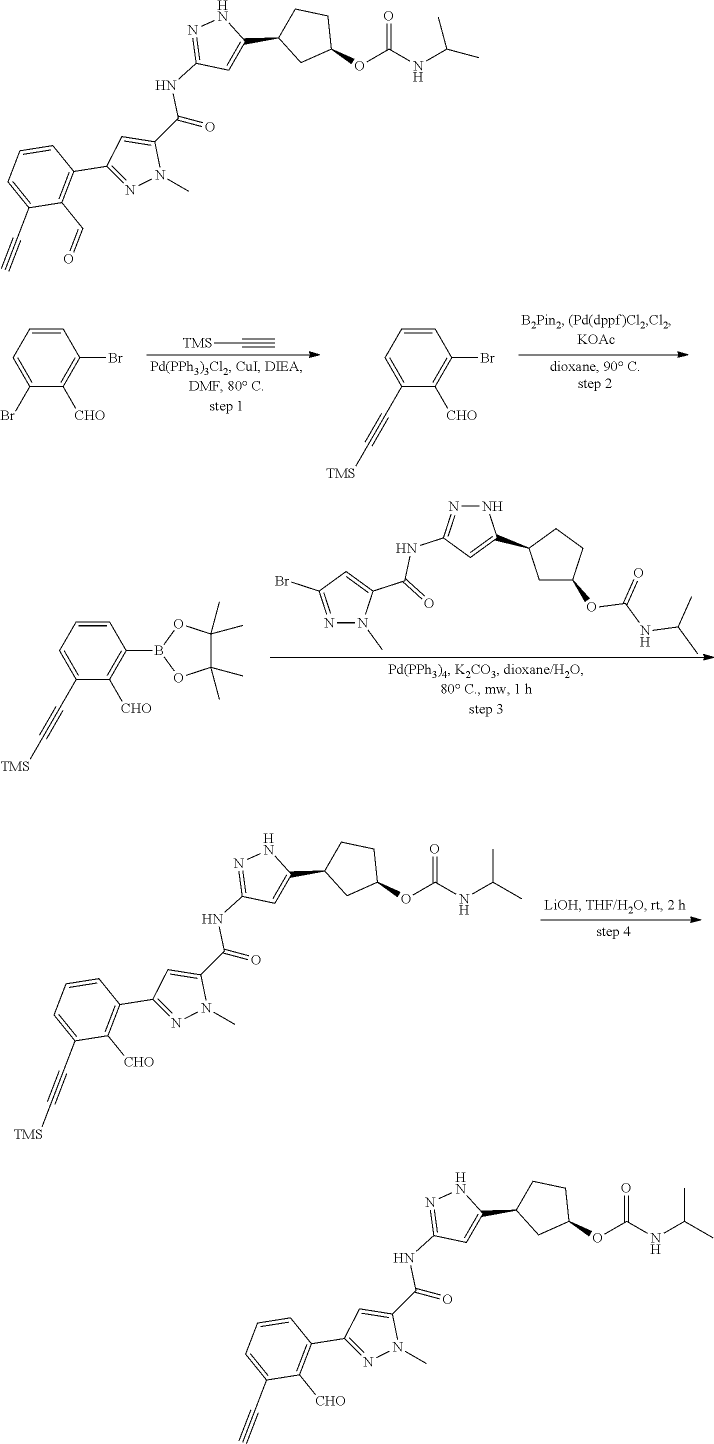

### Step 1: Synthesis of 2-bromo-6-((trimethylsilyl)ethynyl)benzaldehyde

To a stirred solution of 2,6-dibromobenzaldehyde (6.0 g, 22.908 mmol, 1.5 equiv), DIEA (3.9 g, 30.544 mmol, 2 equiv), CuI (0.5 g, 3.054 mmol, 0.2 equiv), and Pd(PPh₃)₂Cl₂ (1.0 g, 1.527 mmol, 0.1 equiv) in DMF (60 mL) at room temperature under N₂ atmosphere. trimethylsilylacetylene (1.5 g, 15.272 mmol, 1.00 equiv) was added to the mixture. The resulting mixture was stirred for 12 h at 80° C. under N₂ atmosphere. The resulting mixture was diluted with water and extracted with EA (3×50 mL). The combined organic layers were washed with brine (2×100 mL), dried over anhydrous Na₂SO₄. After filtration, the filtrate was concentrated under reduced pressure. The residue was purified by silica gel column chromatography, eluted with (0-10% PE/EA) to afford 2-bromo-6-[2-(trimethylsilyl)ethynyl]benzaldehyde (2.1 g, 43.89%) as a yellow oil. MS (ESI) calcd. for C₁₂H₁₃BrOSi, 279.99 m/z, found 280.90 [M+H]⁺.

### Step 2: Synthesis of 2-(4,4,5,5-tetramethyl-1,3,2-dioxaborolan-2-yl)-6-((trimethylsilyl) ethynyl)benzaldehyde

To a solution of 2-bromo-6-[2-(trimethylsilyl)ethynyl]benzaldehyde (2.0 g, 7.112 mmol, 1 equiv) in 1,4-dioxane (100 mL) was added bis(pinacolato)diboron (4.5 g, 17.780 mmol, 2.5 equiv), Pd(dppf)Cl₂CH₂Cl₂ (0.5 g, 0.711 mmol, 0.1 equiv), KOAc (1.7 g, 17.780 mmol, 2.5 equiv). After stirring for 6 h at 80° C. under N₂ atmosphere then cooled to room temperature and diluted with water, the resulting mixture was extracted with EA (3×100 mL). The combined organic layers were washed with brine (100 mL), dried over anhydrous Na₂SO₄. After filtration, the filtrate was concentrated under reduced pressure. The crude product was purified by silica gel column chromatography, eluted with (0-50% PE/EA) to afford 2-(4,4,5,5-tetramethyl-1,3,2-dioxaborolan-2-yl)-6-[2-(trimethylsilyl)ethynyl]benzaldehyde (1.2 g, 20.26%) as a brown oil. MS (ESI) calcd. for C₁₈H₂₅BO₃Si, 328.17 m/z, found 329.20 [M+H]⁺.

### Step 3: Synthesis of (1R,3S)-3-(3-(3-(2-formyl-3-((trimethylsilyl)ethynyl)phenyl)-1-methyl-1H-pyrazole-5-carboxamido)-1H-pyrazol-5-yl)cyclopentyl isopropylcarbamate

To a solution of 2-formyl-3-[2-(trimethylsilyl)ethynyl]phenylboronic acid (168.0 mg, 0.682 mmol, 2 equiv) in 1,4-dioxane (1.5 mL)/H₂O (0.3 mL) was added (1R,3S)-3-[5-(5-bromo-2-methylpyrazole-3-amido)-2H-pyrazol-3-yl]cyclopentyl N-isopropylcarbamate (150.0 mg, 0.341 mmol, 1.00 equiv), Pd(PPh₃)₄ (59.1 mg, 0.051 mmol, 0.15 equiv), K₂CO₃ (117.9 mg, 0.853 mmol, 2.5 equiv). The final reaction mixture was irradiated with microwave radiation for 2 h at 80° C. under N₂ atmosphere then cooled to room temperature and diluted with water, the resulting mixture was extracted with EA (3×10 mL). The combined organic layers were washed with brine (20 mL), dried over anhydrous Na₂SO₄. After filtration, the filtrate was concentrated under reduced pressure. The crude product was purified by silica gel column chromatography, eluted with (0-30% PE/EA) to afford (1R,3S)-3-[5-(5-{2-formyl-3-[2-(trimethylsilyl)ethynyl]phenyl}-2-methylpyrazole-3-amido)-2H-pyrazol-3-yl]cyclopentyl N-isopropylcarbamate (80.0 mg, 32.59%) as a brown oil. MS (ESI) calcd. for C₂₉H₃₆N₆O₄Si, 560.26 m/z, found 561.20 [M+H]⁺.

### Step 4: Synthesis of (1R,3S)-3-{5-[5-(3-ethynyl-2-formylphenyl)-2-methylpyrazole-3-amido]-2H-pyrazol-3-yl}cyclopentyl N-isopropylcarbamate; trifluoroacetic acid

To a stirred solution of (1R,3S)-3-[5-(5-{2-formyl-3-[2-(trimethylsilyl)ethynyl]phenyl}-2-methylpyrazole-3-amido)-2H-pyrazol-3-yl]cyclopentyl N-isopropylcarbamate (60.0 mg, 0.107 mmol, 1 equiv) in THF (1.5 mL) and H₂O (1.5 mL) was added LiOH (7.6 mg, 0.321 mmol, 3 equiv) at room temperature. The resulting mixture was stirred for 1 h. The residue was dissolved in EA (5 mL). The organic layer was extracted with H₂O (3×5 mL). The resulting mixture was concentrated under reduced pressure. The crude product was purified by Prep-HPLC with the following conditions: (Column: X Bridge Shield RP18 OBD Column, 30×150 mm, 5 um; Mobile Phase A: Water (0.05% TFA), Mobile Phase B: ACN; Flow rate: 60 mL/min; Gradient: 38% B to 60% B in 10 min; Wave Length: 254 nm/220 nm; RT1 (min): 9) to afford (1R,3S)-3-{5-[5-(3-ethynyl-2-formylphenyl)-2-methylpyrazole-3-amido]-2H-pyrazol-3-yl}cyclopentyl N-isopropyl carbamate; trifluoroacetic acid (6.7 mg, 9.69%) as a white solid. MS (ESI) calcd. for C₂₆H₂₈N₆O₄, 488.22 m/z, found 489.20 [M+H]⁺. ¹H NMR (300 MHz, DMSO-d₆) δ (ppm): 10.94 (s, 1H), 10.17 (s, 1H), 7.62-7.81 (m, 3H), 7.50 (s, 1H), 6.94-6.96 (m, 1H), 6.44 (s, 1H), 4.99-5.00 (m, 1H), 4.38 (s, 1H), 4.14 (s, 3H), 3.54-3.60 (m, 1H), 3.05-3.10 (m, 1H), 2.48-2.49 (m, 1H), 2.02-2.04 (m, 1H), 1.85-1.99 (m, 1H), 1.58-1.74 (m, 3H), 1.02 (d, J=6.6 Hz, 6H). ¹⁹F NMR (282 MHz, DMSO-d₆) δ (ppm): −74.77.

### Example 7.8: Synthesis of Compound 59: (1R,3S)-3-{5-[5-(2-formyl-3-hydroxyphenyl)-1-methylpyrazole-3-amido]-2H-pyrazol-3-yl}cyclopentyl N-isopropylcarbamate

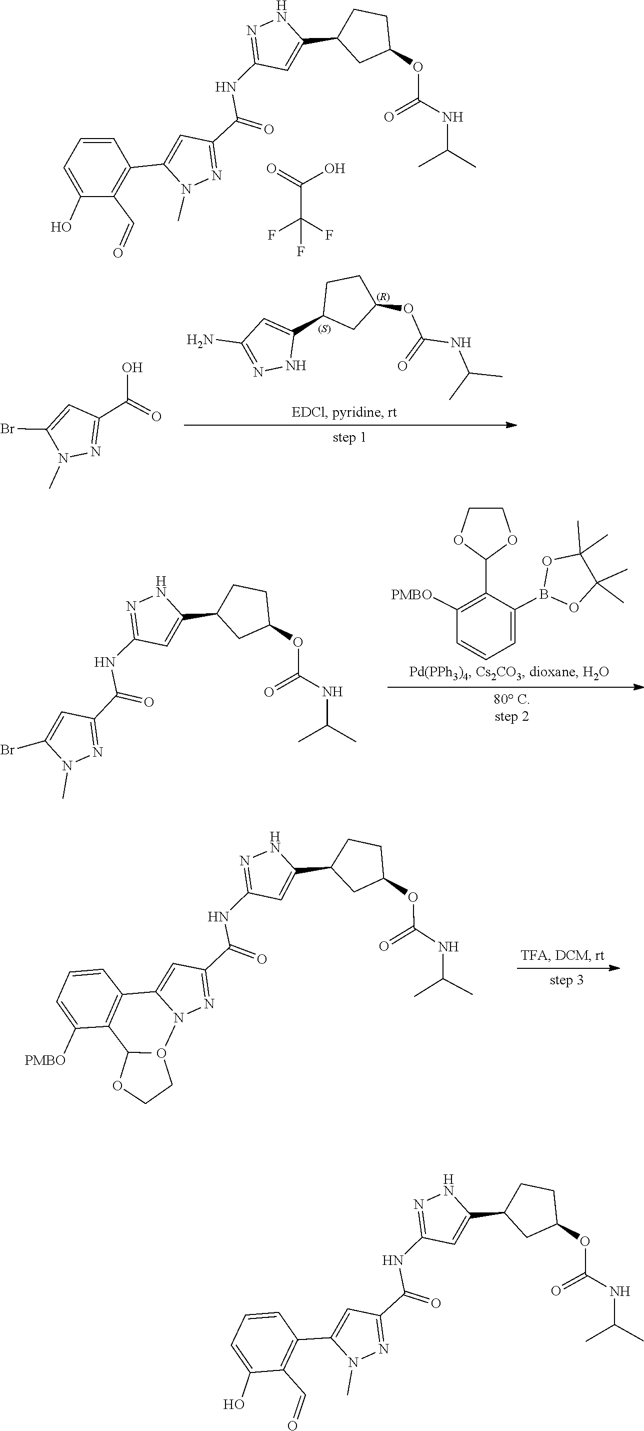

### Step 1: Synthesis of (1R,3S)-3-[5-(5-bromo-1-methylpyrazole-3-amido)-2H-pyrazol-3-yl]cyclopentyl N-isopropylcarbamate

To a stirred solution of 5-bromo-1-methylpyrazole-3-carboxylic acid (0.16 g, 0.780 mmol, 1 equiv) and (1R,3S)-3-(5-amino-2H-pyrazol-3-yl)cyclopentyl N-isopropylcarbamate (0.20 g, 0.780 mmol, 1 equiv) in pyridine (5 mL) was added EDCI (0.60 g, 3.120 mmol, 4 equiv) at 25° C. The resulting mixture was stirred for 3 h at 25° C. The resulting mixture was diluted with water and extracted with EA (3×30 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; ACN/H₂O (0.05% TFA Modifier), 30% to 45% gradient in 10 min; detector, UV 254 nm. This resulted in (1R,3S)-3-[5-(5-bromo-1-methylpyrazole-3-amido)-2H-pyrazol-3-yl]cyclopentyl N-isopropylcarbamate (0.3 g, 85.04%) as a white solid. MS (ESI) calcd. for C₁₇H₂₃BrN₆O₃, 438.10 m/z, found 439.05 [M+H]⁺.

### Step 2: Synthesis of (1R,3S)-3-(5-{5-[2-(1,3-dioxolan-2-yl)-3-[(4-methoxyphenyl)methoxy]phenyl]-1-methylpyrazole-3-amido}-2H-pyrazol-3-yl)cyclopentyl N-isopropylcarbamate

To a stirred solution of (1R,3S)-3-[5-(5-bromo-1-methylpyrazole-3-amido)-2H-pyrazol-3-yl]cyclopentyl N-isopropylcarbamate (150.0 mg, 0.341 mmol, 1 equiv) and 2-[2-(1,3-dioxolan-2-yl)-3-[(4-methoxyphenyl)methoxy]phenyl]-4,4,5,5-tetramethyl-1,3,2-dioxaborolane (168.93 mg, 0.409 mmol, 1.2 equiv) in dioxane (5 mL) and H₂O (1 mL) were added Pd(PPh₃)₄ (39.46 mg, 0.034 mmol, 0.1 equiv) and Cs₂CO₃ (333.74 mg, 1.023 mmol, 3 equiv) at 25° C. The resulting mixture was stirred for 6 h at 80° C. under N₂ atmosphere, then cooled to room temperature and diluted with water. The resulting mixture was extracted with EA (3×20 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O/ACN, 50% gradient in 10 min; detector, UV 254 nm. This resulted in (1R,3S)-3-(5-{5-[2-(1,3-dioxolan-2-yl)-3-[(4-methoxyphenyl)methoxy]phenyl]-1-methylpyrazole-3-amido}-2H-pyrazol-3-yl)cyclopentyl N-isopropylcarbamate (150.0 mg, 67.73%) as a white solid. MS (ESI) calcd. for C₃₄H₄₀N₆O₇, 644.73 m/z, found 645.35 [M+H]⁺.

### Step 3: Synthesis of (1R,3S)-3-{5-[5-(2-formyl-3-hydroxyphenyl)-1-methylpyrazole-3-amido]-2H-pyrazol-3-yl}cyclopentyl N-isopropylcarbamate

To a stirred solution of (1R,3S)-3-(5-{5-[2-(1,3-dioxolan-2-yl)-3-[(4-methoxyphenyl)methoxy]phenyl]-1-methylpyrazole-3-amido}-2H-pyrazol-3-yl)cyclopentyl N-isopropyl carbamate (80.0 mg, 0.124 mmol, 1 equiv) in DCM (3 mL) were added TFA (1 mL, 13.463 mmol, 108.50 equiv) dropwise at 25° C. The resulting mixture was stirred for 0.5 h at 25° C. The reaction was monitored by LCMS. The resulting mixture was concentrated under reduced pressure. The crude product was purified by Prep-HPLC with the following conditions (Column: YMC Triart C18 ExRs, 30×150 mm, 5 μm; Mobile Phase A: Water (10 mmol/L NH₄HCO₃), Mobile Phase B: ACN; Flow rate: 60 mL/min; Gradient: 32% B to 49% B in 10 min; Wave Length: 254/220 nm to afford (1R,3S)-3-{5-[5-(2-formyl-3-hydroxyphenyl)-1-methylpyrazole-3-amido]-2H-pyrazol-3-yl}cyclopentyl N-isopropylcarbamate (9.7 mg, 12.98%) as a white solid. MS (ESI) calcd. for C₂₆H₂₉F₃N₆O₇, 480.21 m/z, found 481.25 [M+H]. ¹H NMR (300 MHz, DMSO-d₆) δ 11.41 (br s, 1H), 10.05 (s, 2H), 7.66-7.69 (m, 1H), 7.12-7.19 (m, 1H), 6.95-6.99 (m, 2H), 6.87 (s, 1H), 6.37 (s, 1H), 4.95-5.02 (m, 1H), 3.70-3.64 (m, 3H), 3.59-3.61 (m, 1H), 3.09-3.11 (m, 1H), 2.47-2.50 (m, 1H), 2.05-2.08 (m, 1H), 1.86-1.93 (m, 1H), 1.65-1.76 (m, 3H), 0.99-1.08 (m, 6H). ¹⁹F NMR (282 MHz, DMSO-d₆) δ (ppm)−74.91.

### Example 7.9: Synthesis of Compound 68: (1R,3S)-3-[5-(2-{2′-formyl-3′-hydroxy-[1,1′-biphenyl]-4-yl}acetamido)-2H-pyrazol-3-yl]cyclopentyl N-isopropylcarbamate; trifluoroacetic acid

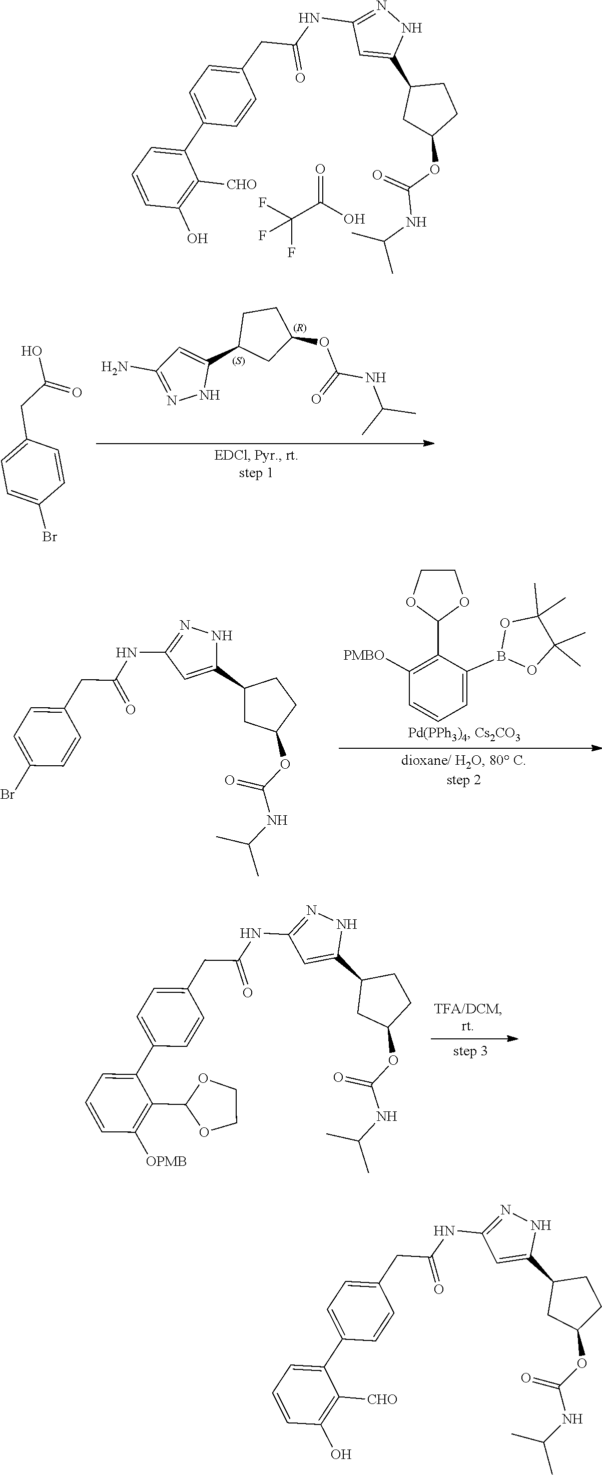

### Step 1: Synthesis of (1R,3S)-3-(3-(2-(4-bromophenyl)acetamido)-1H-pyrazol-5-yl)cyclopentyl isopropylcarbamate

To a stirred solution of 2-(4-bromophenyl)acetic acid (200.0 mg, 0.930 mmol, 1 equiv) and (1R,3S)-3-(5-amino-2H-pyrazol-3-yl)cyclopentyl N-isopropylcarbamate (234.7 mg, 0.930 mmol, 1 equiv) in anhydrous pyridine (5 mL) was added EDCI (423.3 mg, 1.116 mmol, 1.2 equiv). The reaction mixture was stirred at 25° C. for a period of 1 h. Then the resulting mixture was diluted with water and extracted with EA (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE:EA (3:1) to afford (1R,3S)-3-(3-(2-(4-bromophenyl)acetamido)-1H-pyrazol-5-yl)cyclo pentyl isopropylcarbamate (160.0 mg, 38.29%) as a yellow solid. MS (ESI) calcd. for C₂₀H₂₅BrN₄O₃, 448.11 m/z, found 449.10 [M+H]⁺.

### Step 2: Synthesis of (1R,3S)-3-(3-(2-(2′-(1,3-dioxolan-2-yl)-3′-((4-methoxybenzyl)oxy)-[1,1′-biphenyl]-4-yl)acetamido)-1H-pyrazol-5-yl)cyclopentyl isopropylcarbamate

To a solution of (1R,3S)-3-{5-[2-(4-bromophenyl)acetamido]-2H-pyrazol-3-yl}cyclopentyl N-isopropylcarbamate (150.0 mg, 0.334 mmol, 1 equiv) and 2-[2-(1,3-dioxolan-2-yl)-3-[(4-methoxyphenyl)methoxy]phenyl]-4,4,5,5-tetramethyl-1,3,2-dioxaborolane (206.4 mg, 0.501 mmol, 1.5 equiv) in dioxane (1 mL), H₂O (0.2 mL) were added Cs₂CO₃ (271.9 mg, 0.835 mmol, 2.5 equiv) and Pd(PPh₃)₄ (38.6 mg, 0.033 mmol, 0.1 equiv). After stirring for 1 h at 80° C. under nitrogen atmosphere, then cooled to room temperature and diluted with water, the resulting mixture was extracted with EA (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with PE:EA (3:1) to afford (1R,3S)-3-(5-{2-[2′-(1,3-dioxolan-2-yl)-3′-[(4-methoxyphenyl)methoxy]-[1,1′-biphenyl]-4-yl]acetamido}-2H-pyrazol-3-yl)cyclopentyl N-isopropylcarbamate (108.0 mg, 49.41%) as a yellow oil. MS (ESI) calcd. for C₃₇H₄₂N₄O₇, 654.31 m/z, found 655.35 [M+H]⁺.

### Step 3: Synthesis of (1R,3S)-3-[5-(2-{2′-formyl-3′-hydroxy-[1,1′-biphenyl]-4-yl}acetamido)-2H-pyrazol-3-yl]cyclopentyl N-isopropylcarbamate; trifluoroacetic acid

To a stirred solution of (1R,3S)-3-(5-{2-[2′-(1,3-dioxolan-2-yl)-3′-[(4-methoxyphenyl)methoxy]-[1,1′-biphenyl]-4-yl]acetamido}-2H-pyrazol-3-yl)cyclopentyl N-isopropylcarbamate (100.0 mg, 0.153 mmol, 1 equiv) in DCM (3 mL) were added TFA (1 mL) dropwise at room temperature. The resulting mixture was stirred for 0.5 h. The reaction was monitored by LCMS. The resulting mixture was concentrated under reduced pressure. The crude product was purified by Prep-HPLC with the following conditions (Column: Xselect CSH OBD Column, 30*150 mm, 5 um; Mobile Phase A: Water (0.05% TFA), Mobile Phase B: ACN; Flow rate: 60 mL/min; Gradient: 34% B to 56% B in 10 min; Wave Length: 254/220 nm; RT1 (min): 8.8 to afford (1R,3S)-3-[5-(2-{2′-formyl-3′-hydroxy-[1,1′-biphenyl]-4-yl}acetamido)-2H-pyrazol-3-yl]cyclopentyl N-isopropyl carbamate; trifluoroacetic acid (9.0 mg, 9.71%) as a white solid. MS(ESI) calcd for C₂₉H₃₁F₃N₄O₇, 490.22 m/z, found 491.25 [M+H]⁺. 1H NMR (400 MHz, DMSO-d6) δ 11.67 (br s, 1H), 10.63 (br s, 1H), 9.82 (s, 1H), 7.61-7.65 (m, 1H), 7.39-7.45 (m, 4H), 7.01-7.03 (m, 1H), 6.92-6.95 (m, 2H), 6.30 (s, 1H), 4.99-5.00 (m, 1H), 3.68-3.74 (m, 2H), 3.55-3.60 (m, 1H), 3.01-3.08 (m, 1H), 2.46-2.47 (m, 1H), 1.83-2.00 (m, 2H), 1.55-1.82 (m, 3H), 1.01-1.03 (m, 6H). 19F NMR (376 MHz, DMSO-d6) δ −74.89.

### Example 7.10: Synthesis of Compound 71: (1R,3S)-3-(5-{5-[2-formyl-3-hydroxy-5-(methoxymethyl) phenyl]-2-methylpyrazole-3-amido}-2H-pyrazol-3-yl)cyclopentyl N-isopropylcarbamate; trifluoroacetic acid

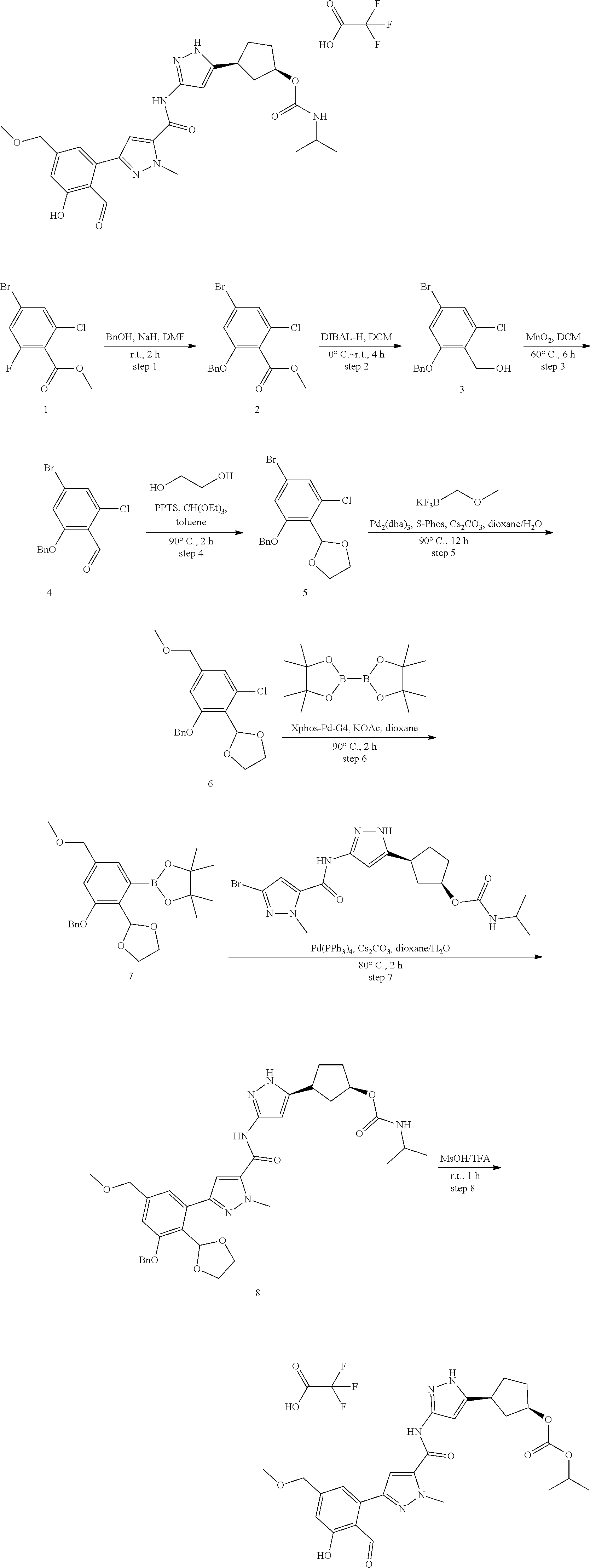

### Step 1: Synthesis of methyl 2-(benzyloxy)-4-bromo-6-chlorobenzoate

To a solution of Benzyl alcohol (4.04 g, 37.386 mmol, 2 equiv) in DMF (100 mL) was added NaH (1.35 g, 56.079 mmol, 3 equiv) at 0° C. and stirred for 1 h at 0° C. Then the mixture was warmed to room temperature, and added methyl 4-bromo-2-chloro-6-fluorobenzoate (5.00 g, 18.693 mmol, 1 equiv). The mixture was stirred at room temperature for 2 h. The reaction was quenched with water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: C18 silica gel column; mobile phase, MeCN in Water (10 mM NH₄HCO₃), 50% to 70% gradient in 10 min; detector, UV 254 nm. to afford methyl 2-(benzyloxy)-4-bromo-6-chlorobenzoate (3.50 g, 36.53%) as an off-white syrup. MS (ESI) calcd. for C₁₅H₁₂BrClO₃, 353.97 m/z, found 353.10 [M−H]⁻.

### Step 2: Synthesis of (2-(benzyloxy)-4-bromo-6-chlorophenyl)methanol

To a stirred solution of methyl 2-(benzyloxy)-4-bromo-6-chlorobenzoate (3.00 g, 8.436 mmol, 1 equiv) in DCM (50 mL) at 0° C. was added DIBAL-H (4.80 g, 33.744 mmol, 4 equiv) in portions. The mixture was let warm to room temperature while stirring for 4 h. The reaction was quenched with sodium sulfate decahydrate at room temperature. The resulting mixture was filtered, the filter cake was washed with CH₂Cl₂ (3×50 mL). The filtrate was concentrated under reduced pressure to afford [2-(benzyloxy)-4-bromo-6-chlorophenyl]methanol (2.00 g, 50.76%) as an off-white semi-solid. ¹H NMR (400 MHz, DMSO-d₆) δ (ppm): 7.45-7.52 (m, 2H), 7.34-7.45 (m, 1H), 7.28-7.33 (m, 4H), 5.20 (s, 2H), 4.57 (s, 2H).

### Step 3: Synthesis of 2-(benzyloxy)-4-bromo-6-chlorobenzaldehyde

To a stirred solution of [2-(benzyloxy)-4-bromo-6-chlorophenyl]methanol (2.00 g, 6.105 mmol, 1 equiv) in DCM (50 mL) was added MnO₂ (3.20 g, 36.809 mmol, 6.03 equiv) in portions at room temperature. The mixture was stirred at 60° C. for 6 h. Desired product could be detected by LCMS. The resulting mixture was filtered, the filter cake was washed with CH₂Cl₂ (3×50 mL). The filtrate was concentrated under reduced pressure to afford 2-(benzyloxy)-4-bromo-6-chlorobenzaldehyde (1.24 g, 62.38%) as a light yellow solid. MS (ESI) calcd. for C₁₄H₁₀BrClO₂, 323.96 m/z, found 325.00 [M+H]⁺.

### Step 4: Synthesis of 2-(2-(benzyloxy)-4-bromo-6-chlorophenyl)-1,3-dioxolane

To a stirred solution of 2-(benzyloxy)-4-bromo-6-chlorobenzaldehyde (0.80 g, 2.457 mmol, 1 equiv) in toluene (16 mL) were added triethyl orthoformate (1.09 g, 7.371 mmol, 3 equiv), ethylene glycol (0.76 g, 12.285 mmol, 5 equiv) and PPTS (0.06 g, 0.246 mmol, 0.1 equiv) in portions at room temperature under nitrogen atmosphere. The mixture was stirred at 90° C. for 12 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (4:6) to afford 2-[2-(benzyloxy)-4-bromo-6-chlorophenyl]-1,3-dioxolane (0.9 g, 89.91%) as a brown yellow solid. MS (ESI) calcd. for C₁₆H₁₄BrClO₃, 369.98 m/z, found 370.90 [M+H]⁺.

### Step 5: Synthesis of 2-(2-(benzyloxy)-6-chloro-4-(methoxymethyl)phenyl)-1,3-dioxolane

To a stirred solution of 2-[2-(benzyloxy)-4-bromo-6-chlorophenyl]-1,3-dioxolane (400.0 mg, 1.082 mmol, 1 equiv) and potassium methoxymethyltrifluoroborate (493.3 mg, 3.246 mmol, 3 equiv) in dioxane (5 mL) and H₂O (5 mL) were added Cs₂CO₃ (881.5 mg, 2.705 mmol, 2.5 equiv), Pd₂(dba)₃ (99.1 mg, 0.108 mmol, 0.1 equiv) and S-Phos (88.9 mg, 0.216 mmol, 0.2 equiv) in portions at room temperature under nitrogen atmosphere. The mixture was stirred at 90° C. for 12 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (2:8) to afford 2-[2-(benzyloxy)-6-chloro-4-(methoxymethyl)phenyl]-1,3-dioxolane (200.0 mg, 52.11%) as a light yellow oil. MS (ESI) calcd. for C₁₈H₁₉ClO₄, 334.10 m/z, found 335.05 [M+H]⁺.

### Step 6: Synthesis of 2-(3-(benzyloxy)-2-(1,3-dioxolan-2-yl)-5-(methoxymethyl)phenyl)-4,4,5,5-tetramethyl-1,3,2-dioxaborolane

To a stirred solution of 2-[2-(benzyloxy)-6-chloro-4-(methoxymethyl)phenyl]-1,3-dioxolane (140.0 mg, 0.418 mmol, 1 equiv) and bis(pinacolato)diboron (424.8 mg, 1.673 mmol, 4.00 equiv) in dioxane (5 mL) were added KOAc (102.6 mg, 1.045 mmol, 2.50 equiv) and XPhos Palladacycle Gen.4 (36.0 mg, 0.042 mmol, 0.10 equiv) in portions at room temperature under nitrogen atmosphere. The mixture was stirred at 90° C. for 12 h. The reaction was quenched with water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (3:7) to afford 2-[3-(benzyloxy)-2-(1,3-dioxolan-2-yl)-5-(methoxymethyl)phenyl]-4,4,5,5-tetramethyl-1,3,2-dioxaborolane (80.0 mg, 29.94%) as a brown solid. MS (ESI) calcd. for C₂₄H₃₁BO₆, 426.22 m/z, found 427.00 [M+H]⁺.

### Step 7: Synthesis of (1R,3S)-3-(3-(3-(3-(benzyloxy)-2-(1,3-dioxolan-2-yl)-5-(methoxymethyl) phenyl)-1-methyl-1H-pyrazole-5-carboxamido)-1H-pyrazol-5-yl)cyclopentyl isopropyl-carbamate

To a stirred solution of 2-[3-(benzyloxy)-2-(1,3-dioxolan-2-yl)-5-(methoxymethyl)phenyl]-4,4,5,5-tetramethyl-1,3,2-dioxaborolane (60.2 mg, 0.141 mmol, 1 equiv) and (1R,3S)-3-[5-(5-bromo-2-methylpyrazole-3-amido)-2H-pyrazol-3-yl]cyclopentyl N-isopropylcarbamate (62.0 mg, 0.141 mmol, 1.00 equiv) in dioxane (2 mL) and H₂O (0.4 mL) were added Cs₂CO₃ (115.0 mg, 0.352 mmol, 2.5 equiv) and Pd(PPh₃)₄ (16.3 mg, 0.014 mmol, 0.1 equiv) in portions at room temperature under nitrogen atmosphere. The mixture was stirred at 90° C. for 2 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mM NH₄HCO₃), 60% to 70% gradient in 10 min; detector, UV 254 nm to afford (1R,3S)-3-(5-{5-[3-(benzyloxy)-2-(1,3-dioxolan-2-yl)-5-(methoxymethyl)phenyl]-2-methylpyrazole-3-amido}-2H-pyrazol-3-yl)cyclopentyl N-isopropylcarbamate (40 mg, 43.03%) as a brown solid. MS (ESI) calcd. for C₃₅H₄₂N₆O₇, 658.31 m/z, found 659.45 [M+H]⁺.

### Step 8: Synthesis of (1R,3S)-3-(5-{5-[2-formyl-3-hydroxy-5-(methoxymethyl)phenyl]-2-methylpyrazole-3-amido}-2H-pyrazol-3-yl)cyclopentyl N-isopropylcarbamate; trifluoroacetic acid

To a stirred solution of (1R,3S)-3-(5-{5-[3-(benzyloxy)-2-(1,3-dioxolan-2-yl)-5-(methoxymethyl) phenyl]-2-methylpyrazole-3-amido}-2H-pyrazol-3-yl)cyclopentyl N-isopropylcarbamate (40.0 mg, 0.061 mmol, 1 equiv) in TFA (0.75 mL) was added methanesulfonic acid (0.25 mL) dropwise at room temperature for 1 h. The crude product was purified by Prep-HPLC with the following conditions (Column: Xselect CSH Prep C18 OBD Column, 30×150 mm, 5 um; Mobile Phase A: Water (0.05% TFA), Mobile Phase B: ACN; Flow rate: 60 mL/min; Gradient: 36% B to 58% B in 10 min) to afford (1R,3S)-3-(5-{5-[2-formyl-3-hydroxy-5-(methoxymethyl)phenyl]-2-methylpyrazole-3-amido}-2H-pyrazol-3-yl)cyclopentyl N-isopropylcarbamate; trifluoroacetic acid (8.8 mg, 22.48%) as a white solid. MS (ESI) calcd. for C₂₂H₃₂N₆O₆, 524.24 m/z, found 525.20 [M+H]⁺. ¹H NMR (400 MHz, DMSO-d₆) δ 11.94 (s, 1H), 10.95 (s, 1H), 10.38 (s, 1H), 7.53 (s, 1H), 7.15 (d, J=1.2 Hz, 1H), 6.93-6.97 (m, 2H), 6.47 (s, 1H), 4.97-5.04 (m, 1H), 4.51 (s, 2H), 4.18 (s, 3H), 3.55-3.63 (m, 1H), 3.37 (s, 3H), 3.08-3.12 (m, 1H), 2.44-2.49 (m, 1H), 2.02-2.08 (m, 1H), 1.88-1.94 (m, 1H), 1.69-1.79 (m, 2H), 1.58-1.67 (m, 1H), 1.04 (d, J=8.0 Hz, 6H). ¹⁹F NMR (376 MHz, DMSO-d₆) δ −74.91.

### Example 7.11: Synthesis of Compound 74: (1R,3S)-3-{5-[5-(2-formyl-3-hydroxy-4-methoxy phenyl)-2-methylpyrazole-3-amido]-2H-pyrazol-3-yl}cyclopentyl N-isopropylcarbamate; trifluoroacetic acid

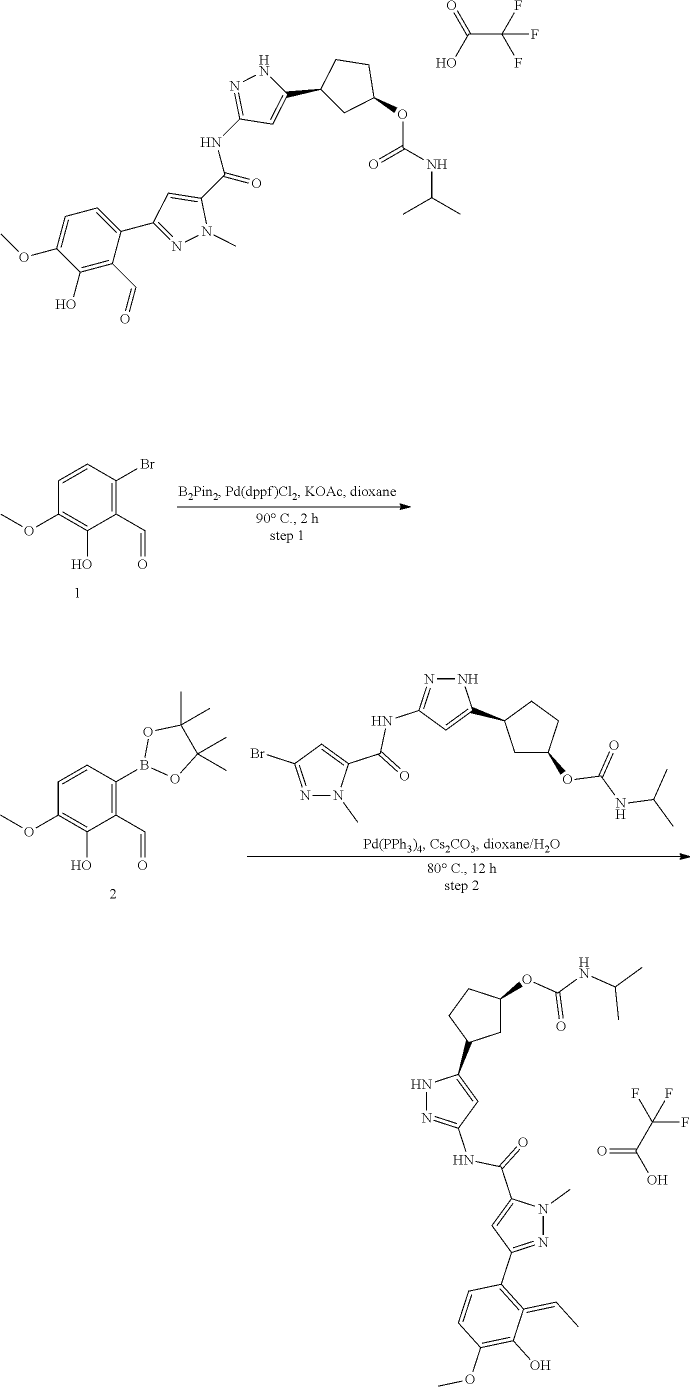

### Step 1: Synthesis of 2-hydroxy-3-methoxy-6-(4,4,5,5-tetramethyl-1,3,2-dioxaborolan-2-yl)benzaldehyde

To a stirred solution 6-bromo-2-hydroxy-3-methoxybenzaldehyde (1.00 g, 4.328 mmol, 1 equiv) and Bis(pinacolato)diboron (4.40 g, 17.312 mmol, 4 equiv) in dioxane (20 mL) were added KOAc (1.06 g, 10.820 mmol, 2.5 equiv) and Pd(dppf)Cl₂ (0.32 g, 0.433 mmol, 0.1 equiv) in portions at room temperature under nitrogen atmosphere. The mixture was stirred at 90° C. for 2 h. The reaction was quenched with Water. The resulting mixture was extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (2:8) to afford 2-hydroxy-3-methoxy-6-(4,4,5,5-tetramethyl-1,3,2-dioxaborolan-2-yl)benzaldehyde (613.0 mg, 46.10%) as a light yellow semi-solid. MS (ESI) calcd. for C₁₄H₁₉BO₅, 278.13 m/z, found 277.15 [M−H]⁻.

### Step 2: (1R,3S)-3-{5-[5-(2-formyl-3-hydroxy-4-methoxyphenyl)-2-methylpyrazole-3-amido]-2H-pyrazol-3-yl}cyclopentyl N-isopropylcarbamate; trifluoroacetic acid

To a stirred solution of 2-hydroxy-3-methoxy-6-(4,4,5,5-tetramethyl-1,3,2-dioxaborolan-2-yl)benzaldehyde (126.6 mg, 0.456 mmol, 2 equiv) and (1R,3S)-3-[5-(5-bromo-2-methylpyrazole-3-amido)-2H-pyrazol-3-yl]cyclopentyl N-isopropylcarbamate (100.0 mg, 0.228 mmol, 1.00 equiv) in dioxane (2.5 mL) and H₂O (0.5 mL) were added Cs₂CO₃ (185.4 mg, 0.570 mmol, 2.5 equiv) and Pd(PPh₃)₄ (26.3 mg, 0.023 mmol, 0.1 equiv) in portions at room temperature under nitrogen atmosphere. The mixture was stirred at 80° C. for 12 h. The crude product (50.0 mg) was purified by Prep-HPLC with the following conditions (Column: XBridge Shield RP18 OBD Column, 30×150 mm, 5 um; Mobile Phase A: Water (0.05% TFA), Mobile Phase B: ACN; Flow rate: 60 mL/min; Gradient: 30% B to 52% B in 10 min; Wave Length: 254 nm/220 nm; RT1 (min): 8) to afford (1R,3S)-3-{5-[5-(2-formyl-3-hydroxy-4-methoxyphenyl)-2-methylpyrazole-3-amido]-2H-pyrazol-3-yl}cyclopentyl N-isopropylcarbamate; trifluoroacetic acid (10.0 mg, 6.91%) as a light yellow solid. MS (ESI) calcd. for C₂₅H₃₀N₆O₆, 510.22 m/z, found 511.25 [M+H]⁺. ¹H NMR (400 MHz, DMSO-d₆) δ 11.98 (br. s, 1H), 10.91 (s, 1H), 10.39 (s, 1H), 7.38-7.42 (m, 2H), 7.12 (d, J=8.4 Hz, 1H), 6.97 (d, J=6.4 Hz, 1H), 6.46 (s, 1H), 4.99-5.03 (m, 1H), 4.15 (s, 3H), 3.87 (s, 3H), 3.56-3.69 (m, 1H), 3.07-3.11 (m, 1H), 2.51-2.53 (m, 1H), 2.02-2.05 (m, 1H), 1.85-1.93 (m, 1H), 1.70-1.79 (m, 2H), 1.58-1.66 (m, 1H), 1.23-1.26 (m, 1H), 1.04 (d, J=6.4 Hz, 6H). ¹⁹F NMR (376 MHz, DMSO-d₆) δ −74.70.

### Example 7.12: Synthesis of Compound 106: (1R,3S)-3-{5-[5-(2-formyl-3-methoxyphenyl)-2-methyl pyrazole-3-amido]-2H-pyrazol-3-yl}cyclopentyl N-isopropylcarbamate; trifluoroacetic acid

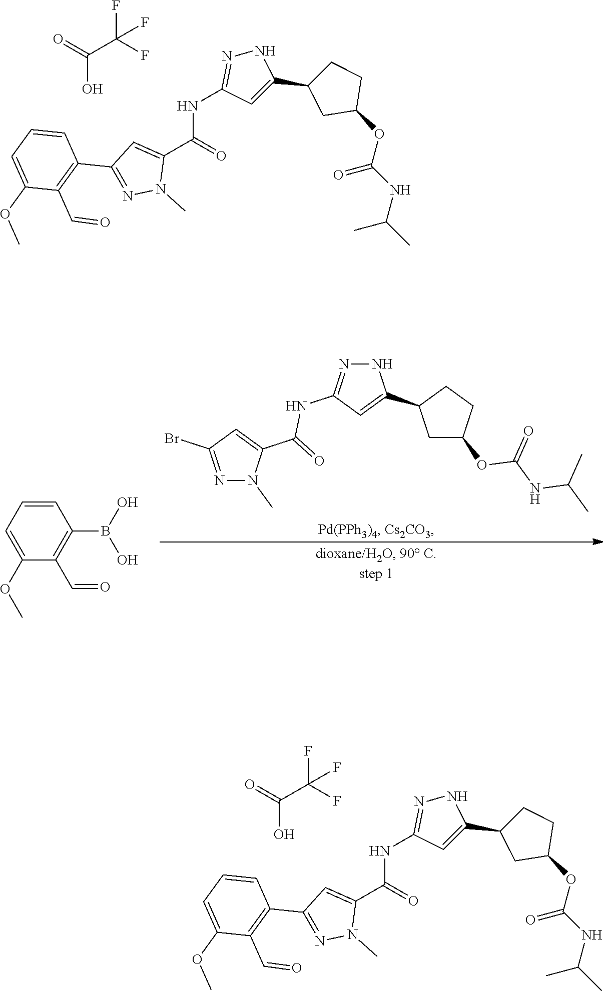

### Step 1: Synthesis of (1R,3S)-3-{5-[5-(2-formyl-3-methoxyphenyl)-2-methylpyrazole-3-amido]-2H-pyrazol-3-yl}cyclopentyl N-isopropylcarbamate; trifluoroacetic acid

To a stirred solution of 2-formyl-3-methoxyphenylboronic acid (159.8 mg, 0.888 mmol, 3 equiv) and (1R,3S)-3-[5-(5-bromo-2-methylpyrazole-3-amido)-2H-pyrazol-3-yl]cyclopentyl N-isopropylcarbamate (130.0 mg, 0.296 mmol, 1.00 equiv) in dioxane (5 mL) and H₂O (1.25 mL) were added Pd(PPh₃)₄ (34.2 mg, 0.030 mmol, 0.1 equiv) and Cs₂CO₃ (241.0 mg, 0.740 mmol, 2.5 equiv) at room temperature under N₂ atmosphere. The resulting mixture was stirred for 2 h at 90° C. The reaction was monitored by LCMS. The resulting mixture was cooled to room temperature, diluted with water and extracted with EA (3×50 mL). The combined organic layers were washed with brine (2×50 mL), dried over anhydrous Na₂SO₄. After filtration, the filtrate was concentrated under reduced pressure. The crude product was purified by Prep-HPLC with the following conditions (Column: XSelect CSH Prep C18 OBD Column 30×150 mm, 5 μm; Mobile Phase A: Water (0.05% TFA), Mobile Phase B: ACN; Flow rate: 60 mL/min; Gradient: 28% B to 47% B in 10 min; Wave Length: 254/220 nm) to afford (1R,3S)-3-{5-[5-(2-formyl-3-methoxyphenyl)-2-methylpyrazole-3-amido]-2H-pyrazol-3-yl}cyclopentyl N-isopropylcarbamate; trifluoroacetic acid (9.6 mg, 5.25%) as a white solid. MS (ESI) calcd. for C₂₅H₃₀N₆O₅, 494.23 m/z, found 495.25 [M+H]⁺. ¹H NMR (400 MHz, DMSO-d₆) δ 10.90 (s, 1H), 10.17 (s, 1H), 7.60-7.64 (m, 1H), 7.43 (s, 1H), 7.18-7.24 (m, 2H), 6.96-6.98 (m, 1H), 6.45 (s, 1H), 5.01-5.02 (m, 1H), 4.13-4.14 (m, 3H), 3.84-3.85 (m, 3H), 3.54-3.63 (m, 1H), 3.07-3.10 (m, 1H), 2.46-2.48 (m, 1H), 2.02-2.07 (m, 1H), 1.85-1.95 (m, 1H), 1.70-1.76 (m, 2H), 1.59-1.66 (m, 1H), 1.04 (d, J=6.4 Hz, 6H). ¹⁹F NMR (376 MHz, DMSO-d₆) δ −74.97.

### Example 8 Representative Schemes Following General Scheme G

### Example 8.1: Synthesis of Compound 76: (1R,3S)-3-(3-((4-(2-formyl-3-hydroxy-5-methoxyphenoxy) 476yridine-2-yl)amino)-1H-pyrazol-5-yl)cyclopentyl isopropylcarbamate

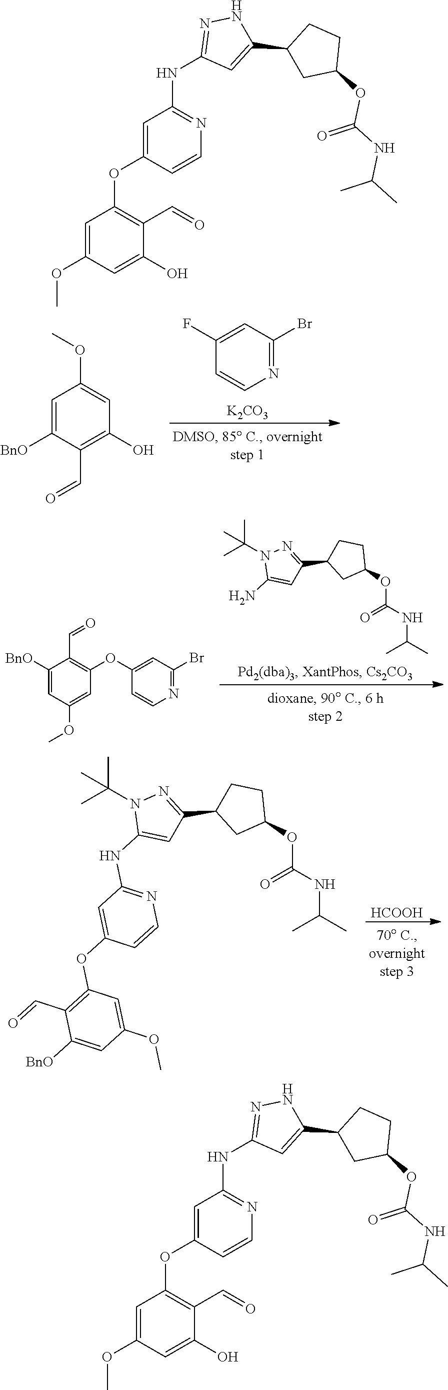

### Step 1: Synthesis of 2-(benzyloxy)-6-((2-bromopyridin-4-yl)oxy)-4-methoxybenzaldehyde

2-(benzyloxy)-6-hydroxy-4-methoxybenzaldehyde (300 mg, 1.162 mmol), 2-bromo-4-fluoropyridine (256 mg, 1.455 mmol), a stir bar and DMSO (7 mL) were added to a 40 vial and stirred until homogeneous, and then treated with K₂CO₃ (495 mg, 3.582 mmol). The reaction mixture was stirred overnight at 85° C., then cooled to r.t and concentrated under vacuum. The reaction mixture diluted with water and extracted with EA (2×50 mL). The combined organic layers were washed with brine, dried over anhydrous sodium sulfate, filtered, and concentrated under vacuum. The residue was purified by silica gel chromatography (0-80% EA/PE) to afford 2-(benzyloxy)-6-[(2-bromopyridin-4-yl)oxy]-4-methoxybenzaldehyde as a light yellow solid (220 mg, 45.72% yield). MS (ESI) mass calcd. for C₂₀H₁₆BrNO₄, 413.02 m/z, found, 414.05 [M+H]⁺.

### Step 2: Synthesis of (1R,3S)-3-(5-((4-(3-(benzyloxy)-2-formyl-5-methoxyphenoxy)477yridine-2-yl)amino)-1-(tert-butyl)-1H-pyrazol-3-yl)cyclopentyl isopropylcarbamate

2-(benzyloxy)-6-[(2-bromopyridin-4-yl)oxy]-4-methoxybenzaldehyde (162 mg, 0.391 mmol), (1R,3S)-3-(5-amino-1-(tert-butyl)-1H-pyrazol-3-yl)cyclopentyl isopropylcarbamate (100 mg, 0.324 mmol), a stir bar, dioxane (5 mL) were added to a 40 vial and stirred until homogeneous, and then treated with Cs₂CO₃ (265 mg, 0.811 mmol), XantPhos (57 mg, 0.099 mmol), Pd₂(dba)₃ (45 mg, 0.049 mmol). The resulting mixture was mixture was stirred at 90° C. for 6 h under nitrogen, then cooled to r.t and diluted with water. The resulting mixture was extracted with extracted with EA (100 mL) twice. The combined organic layers were washed with brine, dried over anhydrous sodium sulfate, filtered, and concentrated under vacuum. The residue was purified by silica gel chromatography (0-20% EtOAc/PE) to afford (1R,3S)-3-(5-((4-(3-(benzyloxy)-2-formyl-5-methoxyphenoxy)478yridine-2-yl)amino)-1-(tert-butyl)-1H-pyrazol-3-yl)cyclopentyl isopropylcarbamate as a white solid (68 mg, 32.68% yield). MS (ESI) mass calcd. for C₃₆H₄₃N₅O₆, 641.32 m/z, found, 642.30 [M+H]⁺.

### Step 3: Synthesis of (1R,3S)-3-(5-([4-(2-formyl-3-hydroxy-5-methoxyphenoxy)478yridine-2-yl]amino-2H-pyrazol-3-yl)cyclopentyl N-isopropylcarbamate

(1R,3S)-3-(5-((4-(3-(benzyloxy)-2-formyl-5-methoxyphenoxy)478yridine-2-yl)amino)-1-(tert-butyl)-1H-pyrazol-3-yl)cyclopentyl isopropylcarbamate (68 mg, 0.106 mmol), a stir bar and HCOOH (4 mL) were added to a 20 mL vial and stirred until homogeneous. The resulting mixture was stirred overnight at 80° C., then cooled to r.t, concentrated under vacuum and purified by Prep-HPLC with (Column: YMC Triart C18 ExRs, 30×150 mm, 5 μm; Mobile Phase A: Water (10 mmol/L NH₄HCO₃), Mobile Phase B: ACN; Flow rate: 60 mL/min; Gradient: 42% B to 64% B in 9 min; Wave Length: 254 nm/220 nm; RT1 (min): 7.5) to afford (1R,3S)-3-(5-([4-(2-formyl-3-hydroxy-5-methoxyphenoxy)478yridine-2-yl]amino-2H-pyrazol-3-yl)cyclopentyl N-isopropyl-carbamate as a white solid (5.4 mg, 10.03% yield, MS (ESI) mass calcd. for C₂₅H₂₉N₅O₆, 495.21 m/z, found, 496.15 [M+H]⁺. ¹H NMR (400 MHz, DMSO-d₆) δ 12.06-11.68 (m, 2H), 10.00 (s, 1H), 9.34-9.11 (m, 1H), 8.07 (d, J=5.7 Hz, 1H), 7.15-6.86 (m, 2H), 6.54-6.34 (m, 2H), 6.27-6.15 (m, 1H), 6.03 (s, 1H), 4.99 (s, 1H), 3.82 (s, 3H), 3.68-3.45 (m, 1H), 3.12-2.92 (m, 1H), 2.49-2.37 (m, 1H), 2.08-1.85 (m, 2H), 1.85-1.46 (m, 3H), 1.04 (dd, J=6.5, 1.7 Hz, 6H).

### Example 8.2: Synthesis of Compound 13: (1R,3S)-3-(5-{[4-(2-formyl-3-hydroxyphenoxy)pyridin-2-yl]amino}-2H-pyrazol-3-yl)cyclopentyl N-isopropylcarbamate; trifluoroacetic acid

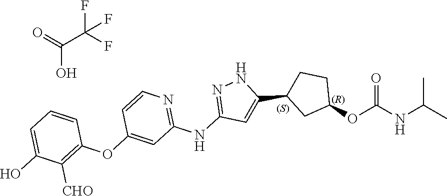

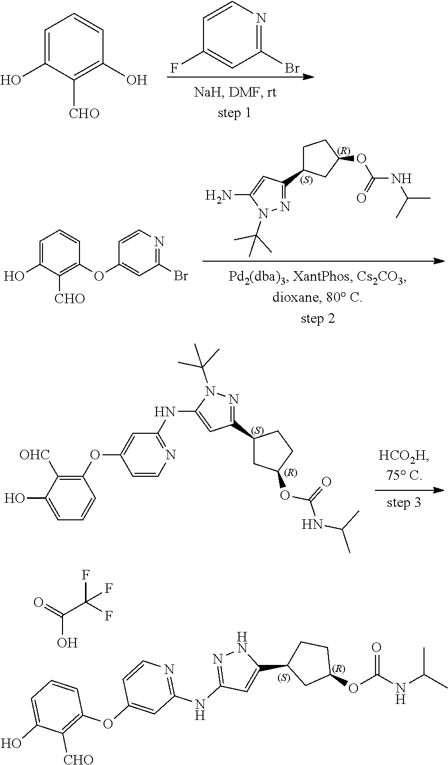

### Step 1: Synthesis of 2-[(2-bromopyridin-4-yl)oxy]-6-hydroxybenzaldehyde

To a stirred solution of 2,6-dihydroxybenzaldehyde (470.9 mg, 3.409 mmol, 1.2 equiv) in DMF (10 mL) was added NaH (102.3 mg, 4.261 mmol, 1.5 equiv) at 0° C. The resulting mixture was stirred for 15 min at 0° C. To the above mixture was added 2-bromo-4-fluoropyridine (500.0 mg, 2.841 mmol, 1.00 equiv) at 0° C., then the resulting mixture was warmed to room temperature. The resulting mixture was stirred for additional 1 h at room temperature. Then the reaction was cooled to 0° C. and quenched with sat. NH₄Cl solution at 0° C. The resulting mixture was extracted with EA (3×100 mL). The combined organic layers were washed with brine (3×100 mL), dried over anhydrous Na₂SO₄. After filtration, the filtrate was concentrated under reduced pressure. This resulted in 2-[(2-bromopyridin-4-yl)oxy]-6-hydroxybenzaldehyde (150.0 mg, 17.95%) as light yellow oil. MS (ESI) calcd. for C₁₂H₈BrNO₃, 292.97 m/z, found 295.95 [M+H]⁺.

### Step 2: Synthesis of (1R,3S)-3-(1-tert-butyl-5-{[4-(2-formyl-3-hydroxyphenoxy)pyridin-2-yl]amino}pyrazol-3-yl)cyclopentyl N-isopropylcarbamate

To a stirred solution of 2-[(2-bromopyridin-4-yl)oxy]-6-hydroxybenzaldehyde (150.0 mg, 0.514 mmol, 1.2 equiv) and (1R,3S)-3-(5-amino-1-(tert-butyl)-1H-pyrazol-3-yl)cyclopentyl isopropyl-carbamate (132.0 mg, 0.428 mmol, 1.00 equiv) in dioxane (5 mL) were added Pd₂(dba)₃ (58.8 mg, 0.064 mmol, 0.15 equiv), XantPhos (74.3 mg, 0.128 mmol, 0.3 equiv) and Cs₂CO₃ (348.6 mg, 1.070 mmol, 2.5 equiv) at room temperature under N₂ atmosphere. The resulting mixture was stirred for 2 h at 80° C., then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by reversed-phase flash chromatography with the following conditions: column, C18 silica gel; mobile phase, ACN in H₂O, 10% to 50% gradient in 10 min; detector, UV 254 nm. This resulted in (1R,3S)-3-(1-(tert-butyl)-5-((4-(2-formyl-3-hydroxyphenoxy)pyridin-2-yl)amino)-1H-pyrazol-3-yl)cyclopentyl isopropylcarbamate (110.0 mg, 49.27%) as alight yellow solid. MS (ESI) calcd. for C₂₈H₃₅N₅O₅, 521.26 m/z, found 522.25 [M+H]⁺.

### Step 3: Synthesis of (1R,3S)-3-(5-{[4-(2-formyl-3-hydroxyphenoxy)pyridin-2-yl]amino}-2H-pyrazol-3-yl)cyclopentyl N-isopropylcarbamate; trifluoroacetic acid

A solution of (1R,3S)-3-(1-(tert-butyl)-5-((4-(2-formyl-3-hydroxyphenoxy)pyridin-2-yl)amino)-1H-pyrazol-3-yl)cyclopentyl isopropylcarbamate (110.0 mg, 0.211 mmol, 1 equiv) in formic acid (3 mL) was stirred for 2 h at 75° C. The resulting mixture was concentrated under reduced pressure. The crude product was purified by Prep-HPLC with the following conditions (Column: XSelect CSH C18 Column, 30×150 mm, 5 μm; Mobile Phase A: Water (0.05% TFA), Mobile Phase B: ACN; Flow rate: 60 mL/min; Gradient: 18% B to 48% B in 7 min; Wave Length: 254 nm) to afford (1R,3S)-3-(5-{[4-(2-formyl-3-hydroxyphenoxy)pyridin-2-yl]amino}-2H-pyrazol-3-yl)cyclopentyl N-isopropylcarbamate (10.6 mg, 10.74%) as a white solid. MS (ESI) calcd. for C₂₄H₂₇N₅O₅, 465.20 m/z, found 466.15 [M+H]⁺. ¹H NMR (400 MHz, DMSO-d₆) δ 11.48 (br s, 1H), 11.02 (br s, 1H), 10.25 (s, 1H), 8.21 (d, J=6.8 Hz, 1H), 7.65-7.69 (m, 1H), 7.04-7.06 (m, 1H), 6.93-6.95 (m, 1H), 6.80-6.85 (m, 2H), 6.52-6.53 (m, 1H), 5.87 (s, 1H), 4.97-5.01 (m, 1H), 3.55-3.60 (m, 1H), 3.09-3.14 (m, 1H), 2.45-2.47 (m, 1H), 2.01-2.07 (m, 1H), 1.87-1.96 (m, 1H), 1.66-1.74 (m, 2H), 1.57-1.63 (m, 1H), 1.02 (d, J=6.4 Hz, 6H). ¹⁹F NMR (376 MHz, DMSO-d₆) δ −74.02.

### Example 8.3: Synthesis of Compound 25: (1R,3S)-3-[5-({4-[(2-formyl-3-hydroxyphenyl)methoxy]pyridine-2-yl}amino)-2H-pyrazol-3-yl]cyclopentyl N-isopropylcarbamate

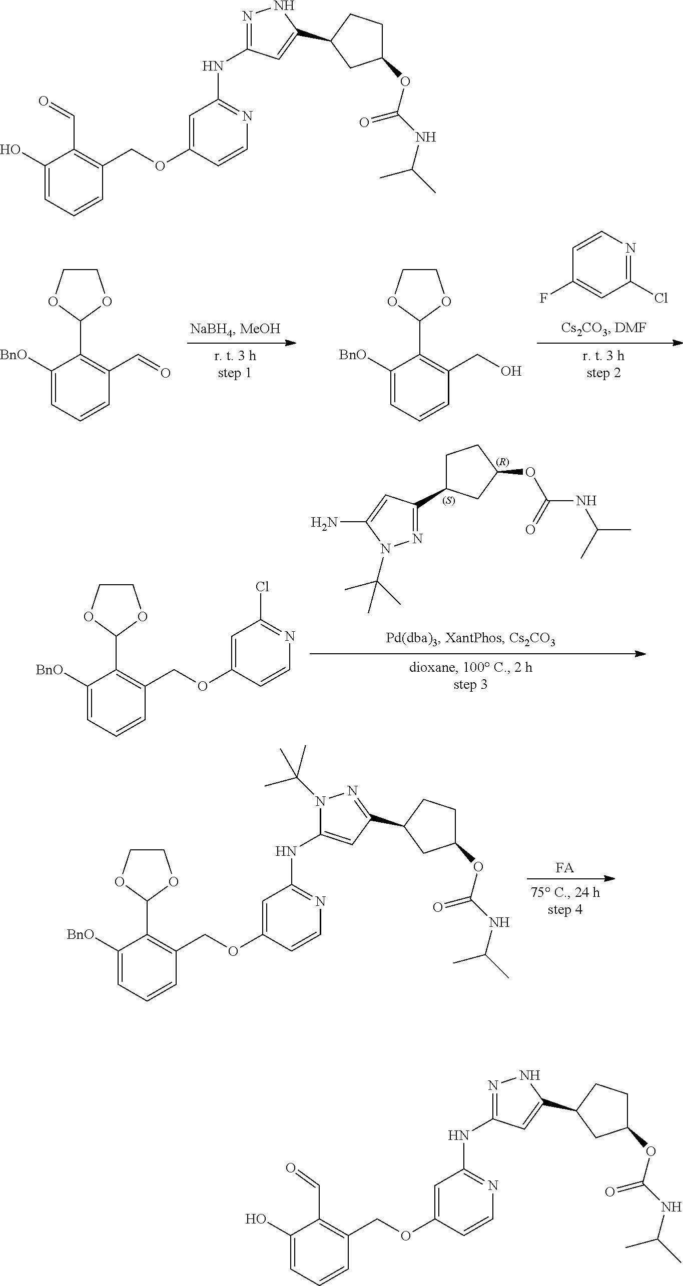

### Step 1: Synthesis of [3-(benzyloxy)-2-(1,3-dioxolan-2-yl)phenyl]methanol

NaBH₄ (66.5 mg, 1.758 mmol, 0.5 equiv) was added to a solution of 3-(Benzyloxy)-2-(1,3-dioxolan-2-yl)benzaldehyde (1.0 g, 3.517 mmol, 1 equiv) in MeOH (15 mL) and stirred at r.t for 3 h. Desired product could be detected by LCMS. The reaction was quenched with H₂O (30 mL), extracted with EA (30 mL×3), washed with brine (10 mL), dried over anhydrous Na₂SO₄, filtered, and concentrated under vacuum to give a nearly yellow viscous oil. The oil was then subjected to silica gel chromatography (0-70% EA/PE) to afford [3-(benzyloxy)-2-(1,3-dioxolan-2-yl)phenyl]methanol (750.0 mg, 67.77%) as a white oil. MS (ESI) calcd. for C₁₇H₁₈O₄, 286.12 m/z, found 287.21 [M+H]⁺.

### Step 2: Synthesis of 4-{[3-(benzyloxy)-2-(1,3-dioxolan-2-yl)phenyl]methoxy}-2-chloropyridine

[3-(Benzyloxy)-2-(1,3-dioxolan-2-yl)phenyl]methanol (800.0 mg, 2.794 mmol, 1 equiv), 2-chloro-4-fluoropyridine (441.0 mg, 3.353 mmol, 1.2 equiv), Cs₂CO₃ (2.3 g, 6.985 mmol, 2.5 equiv) and DMF (5 mL) were added to a 40-mL reaction vial and stirred at r.t for 3 h. Desired product could be detected by LCMS. The reaction mixture was then quenched with H₂O (30 mL), extracted with EA (30 mL×3), washed with brine (10 mL), dried over anhydrous Na₂SO₄, filtered, and concentrated under vacuum to give a nearly yellow viscous oil. The oil was then subjected to silica gel chromatography (0-10% EA/PE) to afford 4-{[3-(benzyloxy)-2-(1,3-dioxolan-2-yl)phenyl]methoxy}-2-chloropyridine (1.0 g, 55.77%) as an off-white solid. MS (ESI) calcd. for C₂₂H₂₀ClNO₄, 397.10 m/z, found 398.05 [M+H]⁺.

### Step 3: Synthesis of (1R,3S)-3-{5-[(4-{[3-(benzyloxy)-2-(1,3-dioxolan-2-yl)phenyl]methoxy} 48lyridine-2-yl)amino]-2H-pyrazol-3-yl}cyclopentyl N-isopropylcarbamate

4-{[3-(Benzyloxy)-2-(1,3-dioxolan-2-yl)phenyl]methoxy}-2-chloropyridine (200.0 mg, 0.503 mmol, 1 equiv), (1R,3S)-3-(5-amino-1-tert-butylpyrazol-3-yl)cyclopentyl N-isopropylcarbamate (186.1 mg, 0.604 mmol, 1.2 equiv), Pd₂(dba)₃ (46.0 mg, 0.050 mmol, 0.1 equiv), XantPhos (58.2 mg, 0.101 mmol, 0.2 equiv), Cs₂CO₃ (409.5 mg, 1.258 mmol, 2.5 equiv) and dioxane (2 mL) were added to a 8 mL vial and stirred at 100° C. for 2 h under N₂ atmosphere. Desired product could be detected by LCMS. The reaction mixture was then quenched with H₂O (10 mL), extracted with EA (10 mL×3), and the combined extracts washed with brine (10 mL), dried over anhydrous Na₂SO₄, filtered, and concentrated under vacuum. The residue was subjected to silica gel chromatography (0-55% EA/PE) to afford (1R,3S)-3-(5-((4-((3-(benzyloxy)-2-(1,3-dioxolan-2-yl)benzyl)oxy)482yridine-2-yl)amino)-1-(tert-butyl)-1H-pyrazol-3-yl)cyclopentyl isopropyl carbamate (150.0 mg, 38.94%) as a yellow solid. MS (ESI) calcd. for C₃₈H₄₇N₅O₆, 669.35 m/z, found 670.45 [M+H]⁺.

### Step 4: Synthesis of (1R,3S)-3-(5-((4-((2-formyl-3-hydroxybenzyl)oxy)482yridine-2-yl)amino)-1H-pyrazol-3-yl)cyclopentyl isopropylcarbamate

(1R,3S)-3-(5-((4-((3-(benzyloxy)-2-(1,3-dioxolan-2-yl)benzyl)oxy)482yridine-2-yl)amino)-1-(tert-butyl)-1H-pyrazol-3-yl)cyclopentyl isopropylcarbamate (100.0 mg, 0.149 mmol, 1 equiv) and FA (2 mL) were added to an 8 mL reaction flask and stirred at 75° C. for 24 h. Desired product could be detected by LCMS. The reaction mixture was purified by Prep-HPLC with the following conditions (Column: Xselect CSH 5 m, 30 mm×150 mm; Mobile Phase A: Water (0.1% FA), Mobile Phase B: ACN; Flow rate: 60 mL/min; Gradient: 20% B to 40% B in 7 min; Wave Length: 254 nm/220 nm nm; RT1 (min): 6.23.) to afford (1R,3S)-3-(5-((4-((2-formyl-3-hydroxybenzyl)oxy) 482yridine-2-yl)amino)-1H-pyrazol-3-yl)cyclopentyl isopropylcarbamate (24.7 mg, 33.40%) as a white solid. MS (ESI) calcd. for C₂₅H₂₉N₅O₅, 479.22 m/z, found 480.25 [M+H]⁺. ¹H NMR (300 MHz, DMSO-d₆) δ 12.65-10.89 (m, 1H), 10.47 (s, 1H), 9.04 (s, 1H), 8.19 (s, 1H), 7.94 (d, J=5.8 Hz, 1H), 7.40-7.59 (m, 1H), 7.07 (d, J=7.5 Hz, 1H), 7.00 (d, J=8.3 Hz, 2H), 6.92 (s, 1H), 6.26-6.45 (m, 1H), 6.00 (s, 1H), 5.43 (s, 2H), 5.00 (s, 1H), 3.49-3.65 (m, 1H), 2.97-3.06 (m, 1H), 2.38-2.49 (m, 1H), 1.94-2.11 (m, 1H), 1.81-1.93 (m, 1H), 1.69-1.70 (m, 2H), 1.60 (s, 1H), 1.03 (d, J=6.6 Hz, 6H).

### Example 8.4: Synthesis of Compound 260: (1R,3S)-3-(3-((5-formyl-6-hydroxyisoquinolin-1-yl)amino)-1H-pyrazol-5-yl)cyclopentyl isopropylcarbamate

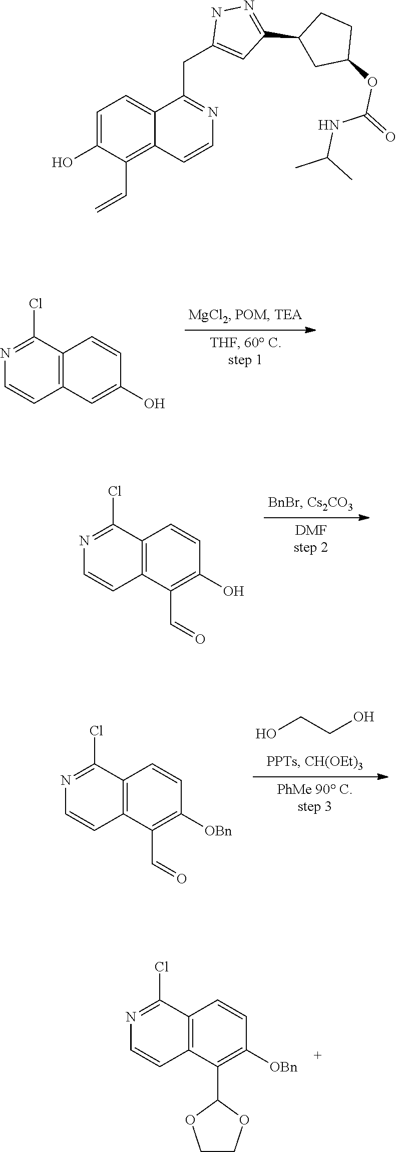

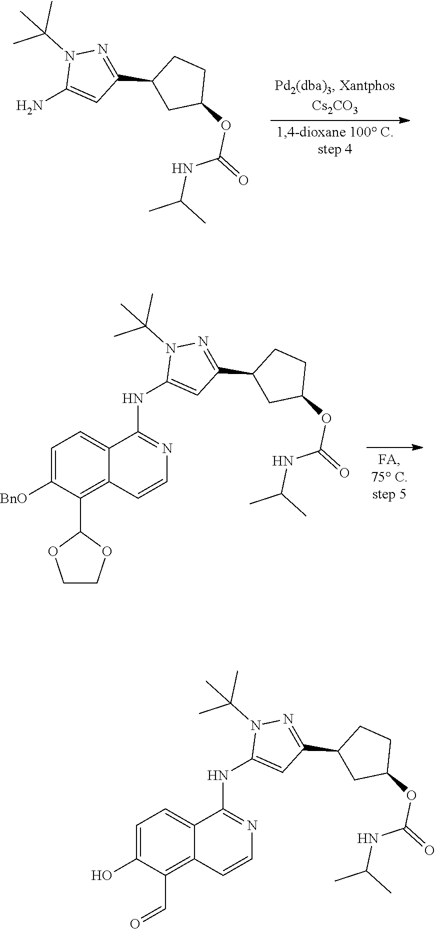 [SMILES: CC(C)NC(=O)O[C@@H]1CC[C@H](c2cc(N)n(C(C)(C)C)n2)C1 ; CC(C)NC(=O)O[C@@H]1CC[C@H](c2cc(Nc3nccc4c(C5OCCO5)c(OCc5ccccc5)ccc34)n(C(C)(C)C)n2)C1 ; CC(C)NC(=O)O[C@@H]1CC[C@H](c2cc(Nc3nccc4c(C=O)c(O)ccc34)n(C(C)(C)C)n2)C1]

### Step 1: Synthesis of 1-chloro-6-hydroxyisoquinoline-5-carbaldehyde

Polyformaldehyde (12.5 g, 139.2 mmol, 5.0 equiv) was added to a solution of 1-chloroisoquinolin-6-ol (5.0 g, 27.8 mmol, 1.0 equiv), MgCl₂ (4.0 g, 41.8 mmol, 1.5 equiv), TEA (7.0 g, 69.6 mmol, 2.5 equiv) in THF (50 mL) and stirred at 60° C. for 16 h. The resulting mixture was acidified to pH 5 with HCl (2 M in H₂O) and extracted with EA (500 mL×3), the organic layers were collected, dried over anhydrous Na₂SO₄, filtered, and concentrated to dryness under vacuum. The crude material was purified by silica gel column chromatography, 0 to 80% EA/PE to afford 1-chloro-6-hydroxyisoquinoline-5-carbaldehyde (5.2 g, 89.0% purity, 80.0% yield) as a light yellow solid. MS (ESI) calcd. for C₁₀H₆ClNO₂ 207.00 m/z, found [M+H]⁺ 208.20 m/z.

### Step 2: Synthesis of 6-(benzyloxy)-1-chloroisoquinoline-5-carbaldehyde

A mixture of 1-chloro-6-hydroxyisoquinoline-5-carbaldehyde (5.2 g, 25.0 mmol, 1.0 equiv) in 30 mL ACN were treated with Cs₂CO₃ (16.3 g, 50.1 mmol, 2.0 equiv) and BnBr (346.0 mg, 2.0 mmol, 1.5 equiv). The resulting mixture was stirred at r.t for 2 h, then diluted with H₂O (500 mL), and extracted with EA (500 mL×3), the organic layers were washed with brine (200 mL), dried over anhydrous Na₂SO₄. After filtration, the filtrate was concentrated to dryness under reduced pressure. The residue was purified by silica gel column chromatography, 0 to 30% EA/PE to afford 6-(benzyloxy)-1-chloroisoquinoline-5-carbaldehyde (3.9 g, 92.0% purity, 48.0% yield) as a yellow solid. MS (ESI) calcd. for C₁₇H₁₂ClNO₂ 297.05 m/z, found [M+H]⁺ 298.20 m/z.

### Step 3: Synthesis of 6-(benzyloxy)-1-chloro-5-(1,3-dioxolan-2-yl)isoquinoline

To a mixture of 6-(benzyloxy)-1-chloroisoquinoline-5-carbaldehyde (3.9 g, 13.0 mmol, 1.0 equiv) in 250 mL toluene was added ethylene glycol (4.04 g, 65.2 mmol, 5.0 equiv), triethyl orthoformate (5.8 g, 39.1 mmol, 3.0 equiv) and PPTs (327.5 mg, 1.3 mmol, 0.1 equiv). The resulting mixture was stirred at 90° C. for 1 h, then diluted with H₂O (500 mL), and extracted with EA (500 mL×3). The combined organic layers were washed with brine (200 mL), dried over anhydrous Na₂SO₄, filtered, and concentrated under reduced pressure. The residue was purified by silica gel column chromatography, 0 to 15% EA/PE to afford 6-(benzyloxy)-1-chloro-5-(1,3-dioxolan-2-yl)isoquinoline (2.60 g, 90% purity, 56.0% yield) as a white solid. MS (ESI) calcd. for C₁₉H₁₆C₁NO₃ 341.08 m/z, found [M+H]⁺ 342.20 m/z.

### Step 4: Synthesis of (1R,3S)-3-(5-((6-(benzyloxy)-5-(1,3-dioxolan-2-yl)isoquinolin-1-yl)amino)-1-(tert-butyl)-1H-pyrazol-3-yl)cyclopentyl isopropylcarbamate

6-(Benzyloxy)-1-chloro-5-(1,3-dioxolan-2-yl)isoquinoline (500.0 mg, 1.5 mmol, 1.0 equiv), Cs₂CO₃ (956.2 mg, 2.9 mmol, 2.0 equiv), (1R,3S)-3-(5-amino-1-(tert-butyl)-1H-pyrazol-3-yl)cyclopentyl isopropylcarbamate (676.8 mg, 2.2 mmol, 1.5 equiv), Xantphos (169.3 mg, 0.3 mmol, 0.2 equiv), Pd₂(dba)₃ (133.9 mg, 0.1 mmol, 0.1 equiv) and 5 mL 1,4-dioxane were added to a 25 mL flask with a stir bar and stirred at 100° C. under N₂ for 2 h. The reaction mixture was then diluted with H₂O (100 mL), extracted with EA (100 mL×3), washed with brine (50 mL), dried over anhydrous Na₂SO₄, filtered, and concentrated under vacuum to give a yellow viscous oil. The residue was purified by silica gel column chromatography, 0 to 71% EA/PE to afford (1R,3S)-3-(5-((6-(benzyloxy)-5-(1,3-dioxolan-2-yl)isoquinolin-1-yl)amino)-1-(tert-butyl)-1H-pyrazol-3-yl)cyclopentyl isopropylcarbamate (510.0 mg, 84.0% purity, 47.0% yield) as a yellow solid. MS (ESI) calcd. for C₃₅H₄₃N₅O₅ 613.159 m/z, found [M+H]⁺ 614.25 m/z.

### Step 5: Synthesis of (1R,3S)-3-(3-((5-formyl-6-hydroxyisoquinolin-1-yl)amino)-1H-pyrazol-5-yl)cyclopentyl isopropylcarbamate

A mixture of (1R,3S)-3-(5-((6-(benzyloxy)-5-(1,3-dioxolan-2-yl)isoquinolin-1-yl)amino)-1-(tert-butyl)-1H-pyrazol-3-yl)cyclopentyl isopropylcarbamate (500.0 mg, 0.8 mmol, 1.0 equiv) and FA (20 mL) with a stir bar in 50 mL flask was stirred at 75° C. for 48 h. The mixture was purified by reverse phase HPLC, 11 to 31% ACN/Water with 10 mmol/L NH₄HCO₃ as modifier to afford (1R,3S)-3-(3-((5-formyl-6-hydroxyisoquinolin-1-yl)amino)-1H-pyrazol-5-yl)cyclopentyl isopropylcarbamate (25.0 mg, 95.0% purity, 6.8% yield) as a yellow solid. ¹H NMR (300 MHz, DMSO) δ 12.11 (s, 2H), 10.65 (s, 1H), 8.62 (s, 1H), 8.17 (s, 1H), 7.99 (s, 1H), 7.06 (s, 1H), 6.97 (d, J=7.8 Hz, 1H), 6.39 (s, 1H), 5.02 (s, 1H), 3.57-3.42 (m, 1H), 3.09 (s, 1H), 2.48-2.39 (m, 1H), 2.11-1.98 (m, 2H), 1.97-1.65 (m, 3H), 1.14-0.88 (m, 7H), MS (ESI) calcd. for C₂₂H₂₅N₅O₄ 423.19 m/z, found [M+H]⁺ 424.15 m/z.

### Example 8.5: Synthesis of Compound 261: (1R,3S)-3-(3-((5-formyl-6-hydroxyisoquinolin-1-yl)amino)-1H-pyrazol-5-yl)cyclopentyl (1-methylcyclopropyl)carbamate

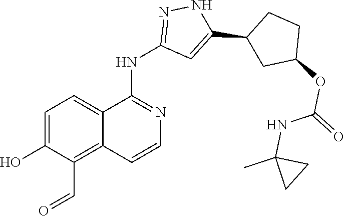 [SMILES: CC1(NC(=O)O[C@@H]2CC[C@H](c3cc(Nc4nccc5c(C=O)c(O)ccc45)n[nH]3)C2)CC1]

### Synthetic Route

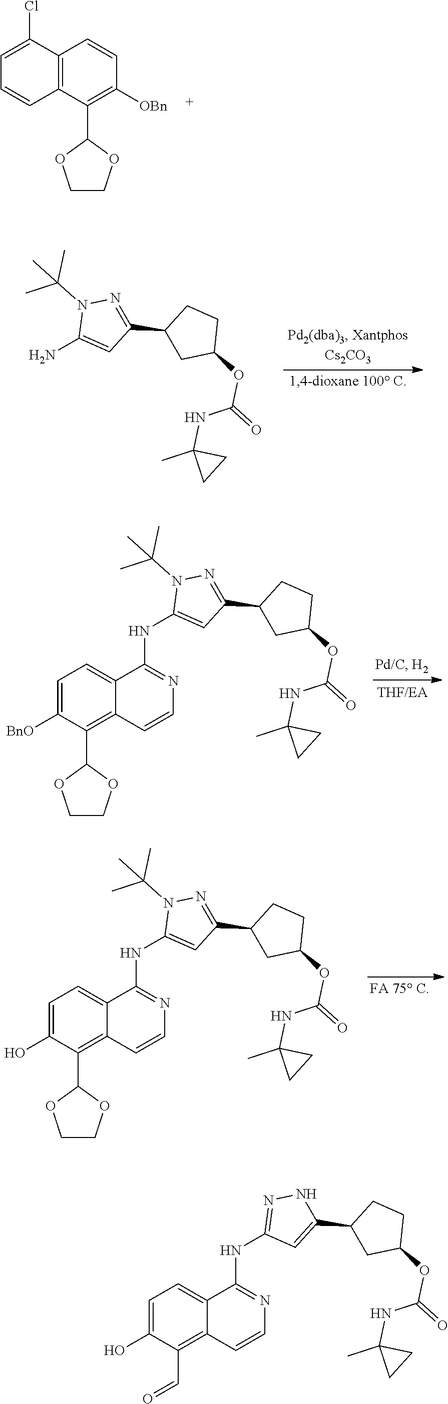

### Step 1: (1R,3S)-3-(5-((6-(benzyloxy)-5-(1,3-dioxolan-2-yl)isoquinolin-1-yl)amino)-1-(tert-butyl)-1H-pyrazol-3-yl)cyclopentyl (1-methylcyclopropyl)carbamate

6-(Benzyloxy)-1-chloro-5-(1,3-dioxolan-2-yl)isoquinoline (210.0 mg, 0.614 mmol, 1 equiv), (1R,3S)-3-(5-amino-1-tert-butylpyrazol-3-yl)cyclopentyl N-(1-methylcyclopropyl)carbamate (295.3 mg, 0.921 mmol, 1.5 equiv), Pd₂(dba)₃ (56.2 mg, 0.061 mmol, 0.1 equiv), Xantphos (71.1 mg, 0.123 mmol, 0.2 equiv), Cs₂CO₃ (400.3 mg, 1.228 mmol, 2 equiv) and 5 mL 1,4-dioxane were added to a 20 mL flask with a stir bar and stirred at 100° C. for 1 h under N₂. The mixture was diluted with 20 mL EA, washed with H₂O (3×20 mL) and brine (20 mL), dried over anhydrous sodium sulfate, filtered, and concentrated to dryness in vacuo. The residue was purified by silica gel column chromatography, 0 to 70% EA/PE to afford (1R,3S)-3-(5-((6-(benzyloxy)-5-(1,3-dioxolan-2-yl)isoquinolin-1-yl)amino)-1-(tert-butyl)-1H-pyrazol-3-yl)cyclopentyl (1-methylcyclopropyl)carbamate as a yellow oil (140.0 mg, 90% purity, 36.41% yield). MS (ESI) calcd. for C₃₆H₄₃N₅O₅, 625.32 m/z, found [M+H]⁺ 626.20 m/z.

### Step 2: (1R,3S)-3-(5-((5-(1,3-dioxolan-2-yl)-6-hydroxyisoquinolin-1-yl)amino)-1-(tert-butyl)-1H-pyrazol-3-yl)cyclopentyl (1-methylcyclopropyl)carbamate

(1R,3S)-3-(5-((6-(benzyloxy)-5-(1,3-dioxolan-2-yl)isoquinolin-1-yl)amino)-1-(tert-butyl)-1H-pyrazol-3-yl)cyclopentyl (1-methylcyclopropyl)carbamate (140.0 mg, 0.224 mmol, 1 equiv), Pd/C (95.2 mg, 0.896 mmol, 4 equiv, 10% on active carbon), 2 mL THF and 2 mL EA were added to a 25 mL flask with a stir bar and stirred for 1 h under H₂ at r.t. The resulting mixture was filtered, the filter cake was washed 10 mL THF. The filtrate was concentrated under reduced pressure to afford (1R,3S)-3-(5-((5-(1,3-dioxolan-2-yl)-6-hydroxyisoquinolin-1-yl)amino)-1-(tert-butyl)-1H-pyrazol-3-yl)cyclopentyl (1-methylcyclopropyl)carbamate (110.0 mg, 90% purity, 91.79% yield) as a colorless oil. MS (ESI) calcd. for C₂₉H₃₇N₅O₅, 535.27 m/z, found [M+H]⁺ 536.20 m/z.

### Step 3: (1R,3S)-3-(3-((5-formyl-6-hydroxyisoquinolin-1-yl)amino)-1H-pyrazol-5-yl)cyclopentyl (1-methylcyclopropyl)carbamate

A solution of (1R,3S)-3-(5-((5-(1,3-dioxolan-2-yl)-6-hydroxyisoquinolin-1-yl)amino)-1-(tert-butyl)-1H-pyrazol-3-yl)cyclopentyl (1-methylcyclopropyl)carbamate (110.0 mg, 0.280 mmol, 1 equiv) in 2 mL FA was stirred at 75° C. for 1 h. The reaction mixture was purified by reverse phase HPLC, 8 to 25% ACN/H₂O with 0.1% FA modifier to afford (1R,3S)-3-(3-((5-formyl-6-hydroxyisoquinolin-1-yl)amino)-1H-pyrazol-5-yl)cyclopentyl (1-methylcyclopropyl)carbamate as a yellow solid (28.5 mg, 31.87% yield). ¹H NMR (300 MHz, DMSO) δ 12.32 (s, 2H), 10.65 (s, 1H), 9.71 (s, 1H), 8.62 (d, J=9.3 Hz, 1H), 8.22-8.01 (m, 1H), 7.99 (s, 1H), 7.37 (s, 1H), 7.07 (d, J=9.4 Hz, 1H), 6.93-6.85 (m, 1H), 6.33 (s, 1H), 5.01 (s, 1H), 3.08 (s, 1H), 2.03 (s, 1H), 1.91 (s, 2H), 1.74 (s, 3H), 1.24 (s, 3H), 0.60 (s, 2H), 0.48 (s, 2H). MS (ESI) calcd. for C₂₃H₂₅N₅O₄, 435.19 m/z, found [M+H]⁺ 436.20 m/z.

### Example 9 Representative Schemes Following General Scheme H

### Example 9.1: Synthesis of Compound 63 (2-formyl-3-hydroxyphenyl)methyl N-{5-[(1S,3R)-3-[(isopropylcarbamoyl)oxy]cyclopentyl]-1H-pyrazol-3-yl}carbamate

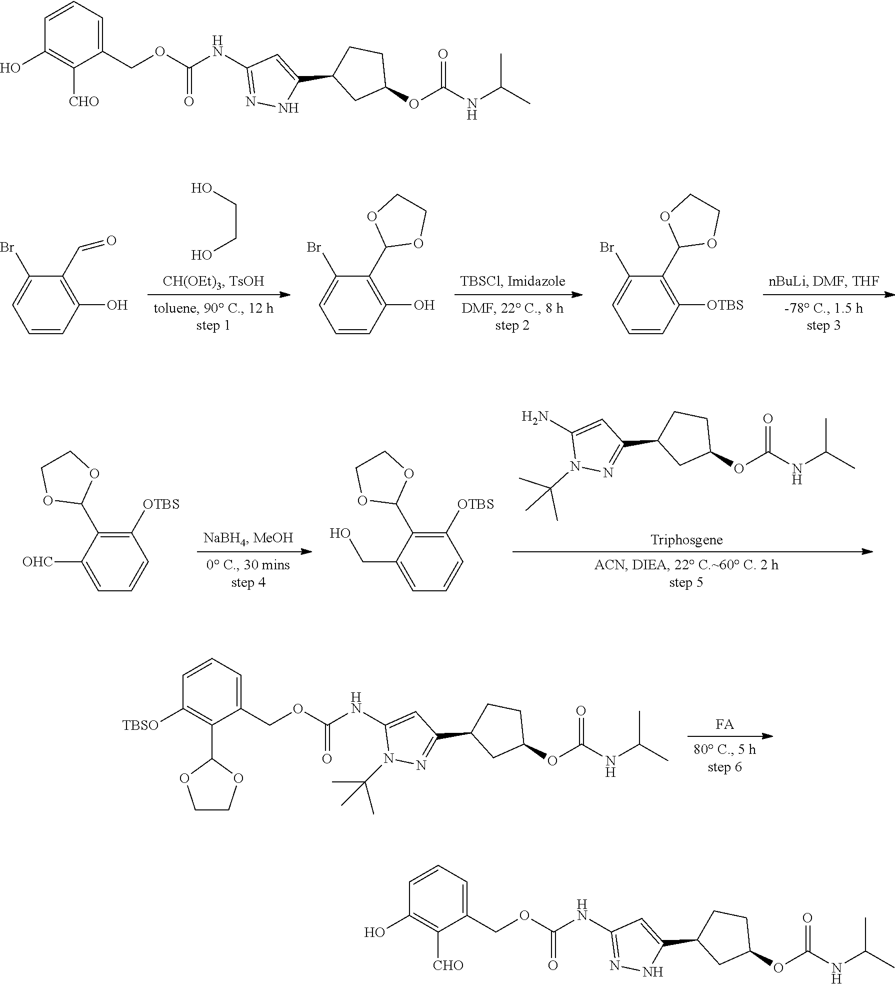

### Step 1: Synthesis of 3-bromo-2-(1,3-dioxolan-2-yl)phenol

2-Bromo-6-hydroxybenzaldehyde (25.0 g, 124.366 mmol, 1 equiv), ethylene glycol (69.47 g, 1119.294 mmol, 9.00 equiv), (diethoxymethoxy)ethane (82.94 g, 559.649 mmol, 4.50 equiv), TsOH (2.14 g, 12.437 mmol, 0.1 equiv) and toluene (250 mL) were added to a 1 L round-bottom flask with a stir bar and stirred at 100° C. for 24 h under N₂. The resulting mixture was diluted with EA (1 L) and extracted with H₂O (3×500 mL). The combined organic layers were washed with brine (1 L), dried over anhydrous Na₂SO₄. After filtration, the filtrate was concentrated under reduced pressure. The residue was purified by silica gel column chromatography, eluted with EA/PE (0-50%) to afford 3-bromo-2-(1,3-dioxolan-2-yl)phenol (22 g, 62.80%) as a purple oil. MS (ESI) calcd. for C₉H₉BrO3, 243.97 m/z, found 244.97 [M+H]⁺.

### Step 2: 3-bromo-2-(1,3-dioxolan-2-yl)phenoxy(tert-butyl)dimethylsilane

To a stirred solution of 3-bromo-2-(1,3-dioxolan-2-yl)phenol (4 g, 16.322 mmol, 1 equiv) and imidazole (3.33 g, 48.966 mmol, 3 equiv) in DMF (40 mL) was added TBSCl (5.90 g, 39.173 mmol, 2.4 equiv) in portions at 0° C. under N₂ atmosphere. The resulting mixture was stirred for additional 8 h at 22° C. The resulting mixture was diluted with EA (500 mL). The resulting mixture was extracted with H₂O (200 mL×3). The combined organic layers were washed with brine (200 mL), dried over anhydrous Na₂SO₄. After filtration, the filtrate was concentrated under reduced pressure. The residue was purified by silica gel column chromatography, eluted with EA/PE (0-50%) to afford 3-bromo-2-(1,3-dioxolan-2-yl)phenoxy(tert-butyl)dimethylsilane (3 g, 45.01%) as a white solid. MS (ESI) calcd. for C₁₅H₂₃BrO₃Si, 360.06 m/z, found 361.05 [M+H]⁺.

### Step 3: 3-[(tert-butyldimethylsilyl)oxy]-2-(1,3-dioxolan-2-yl)benzaldehyde

A solution of 3-bromo-2-(1,3-dioxolan-2-yl)phenoxy(tert-butyl)dimethylsilane (3 g, 8.349 mmol, 1 equiv) in THF (30 mL) was treated with n-BuLi (4.01 mL, 10.019 mmol, 1.2 equiv, 2.5 M) for 30 min at −78° C. under nitrogen atmosphere followed by the addition of DMF (10 mL) dropwise at −78° C. The resulting mixture was stirred for additional 1 h at −78° C. The reaction was quenched by the addition of NHCl₄ (aq. 100 mL) at 0° C. The aqueous layer was extracted with EA (100 mL×3). The combined organic layers were washed with brine (100 mL), dried over anhydrous Na₂SO₄. After filtration, the filtrate was concentrated under reduced pressure. After filtration, the filtrate was concentrated under reduced pressure. The residue was purified by silica gel column chromatography, eluted with EA/PE (0-50%) to afford 3-[(tert-butyldimethylsilyl)oxy]-2-(1,3-dioxolan-2-yl)benzaldehyde (2.6 g, 98.95%) as a yellow solid. MS (ESI) calcd. for C₂₃H₃₁NO₆Si, 445.19 m/z, found 446.19 [M+H]⁺.

### Step 4: {3-[(tert-butyldimethylsilyl)oxy]-2-(1,3-dioxolan-2-yl)phenyl}methanol

To a stirred solution of 3-[(tert-butyldimethylsilyl)oxy]-2-(1,3-dioxolan-2-yl)benzaldehyde (2.6 g, 8.429 mmol, 1 equiv) in MeOH (30 mL) was added NaBH₄ (0.16 g, 4.215 mmol, 0.5 equiv) in portions at 0° C. The resulting mixture was stirred for additional 30 min at 0° C. The reaction was quenched by the addition of NHCl₄ (aq. 10 mL) at 0° C. The aqueous layer was extracted with DCM (20 mL×3). The combined organic layers were washed with brine (10 mL), dried over anhydrous Na₂SO₄. After filtration, the filtrate was concentrated under reduced pressure. The residue was purified by silica gel column chromatography, eluted with EA/PE (0-100%) to afford {3-[(tert-butyldimethylsilyl)oxy]-2-(1,3-dioxolan-2-yl)phenyl}methanol (2.6 g, 90.41%) as a white solid. MS (ESI) calcd. for C₁₆H₂₆O₄Si, 310.16 m/z, found 333.16 [M+Na]⁺.

### Step 5: (1R,3S)-3-{2-tert-butyl-5-[(chlorocarbonyl)amino]pyrazol-3-yl}cyclopentyl N-isopropylcarbamate

A solution of (1R,3S)-3-(5-amino-1-(tert-butyl)-1H-pyrazol-3-yl)cyclopentyl isopropylcarbamate (300 mg, 0.973 mmol, 1 equiv) in ACN (5 mL) was treated with triphosgene (144.3 mg, 0.486 mmol, 0.5 equiv) for 30 min at 22° C. under nitrogen atmosphere followed by the addition of {3-[(tert-butyldimethylsilyl)oxy]-2-(1,3-dioxolan-2-yl)phenyl}methanol (301.9 mg, 0.973 mmol, 1 equiv) dropwise at 60° C. The resulting mixture was stirred for additional 1.5 h at 60° C. Desired product was detected by LCMS. The resulting mixture was diluted with EA (20 mL). The resulting mixture was extracted with H₂O (10 mL×3). The combined organic layers were washed with brine (10 mL), dried over anhydrous Na₂SO₄. After filtration, the filtrate was concentrated under reduced pressure. This resulted in (1R,3S)-3-(1-(tert-butyl)-5-((((3-((tert-butyldimethylsilyl)oxy)-2-(1,3-dioxolan-2-yl)benzyl)oxy)carbonyl)amino)-1H-pyrazol-3-yl)cyclopentyl isopropyl carbamate (460 mg, 25.50%) as a yellow solid which was used for next step directly. MS (ESI) calcd. for C₁₇H₂₇ClN₄O₃, 370.17 m/z, found 371.17 [M+H]⁺.

### Step 6: (2-formyl-3-hydroxyphenyl)methyl N-{5-[(1S,3R)-3-[(isopropylcarbamoyl)oxy]cyclopentyl]-1H-pyrazol-3-yl}carbamate

A solution of (1R,3S)-3-(1-(tert-butyl)-5-((((3-((tert-butyldimethylsilyl)oxy)-2-(1,3-dioxolan-2-yl)benzyl)oxy)carbonyl)amino)-1H-pyrazol-3-yl)cyclopentyl isopropylcarbamate (200 mg, 0.310 mmol, 1 equiv) in formic acid (4 mL) was stirred at 80° C. for 5 h. Desired product could be detected by LCMS. The mixture was purified by Prep-HPLC under the condition: Column: Xbridge Shield RP 18 OBD Column, 19×250 mm, 5 μm; Mobile Phase A: Water (10 mmol/L NH₄HCO₃+0.1% NH₃·H₂O), Mobile Phase B: ACN; Flow rate: 25 mL/min mL/min; Gradient: 37% B to 67% B in 7 min; Wave Length: 254 nm nm; RT1 (min): 6.55 to afford (2-formyl-3-hydroxyphenyl)methyl N-{5-[(1S,3R)-3-[(isopropylcarbamoyl)oxy]cyclopentyl]-1H-pyrazol-3-yl}carbamate (3.3 mg, 2.44%) as a white solid. MS (ESI) calcd. for C₂₁H₂₆N₄O₆, 430.18 m/z, found 431.18 [M+H]⁺. ¹H NMR (400 MHz, DMSO-d₆) δ 12.00 (s, 1H), 11.06 (s, 1H), 10.46 (s, 1H), 9.98 (s, 1H), 7.52 (t, J=8.0 Hz, 1H), 7.15-6.72 (m, 3H), 6.10 (s, 1H), 5.42 (s, 2H), 4.98 (s, 1H), 3.57 (q, J=6.8 Hz, 1H), 3.07-2.98 (m, 1H), 2.48-2.41 (m, 1H) 2.03-1.94 (m, 1H), 1.94-1.83 (m, 1H), 1.74-1.64 (m, 2H), 1.59-1.56 (m, 1H) 1.02 (dd, J=6.7, 2.0 Hz, 6H).

### Example 9.2: Synthesis of Compound 78: (1R,3S)-3-(3-(3-(2-formyl-3-hydroxyphenyl)-2,5-dihydro-1H-pyrrole-1-carboxamido)-1H-pyrazol-5-yl)cyclopentyl isopropylcarbamate

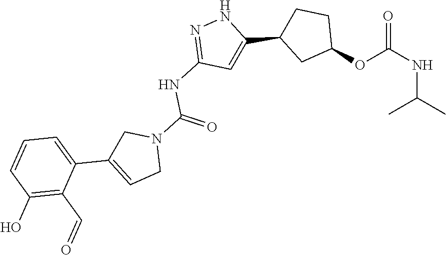 [SMILES: CC(C)NC(=O)O[C@@H]1CC[C@H](c2cc(NC(=O)N3CC=C(c4cccc(O)c4C=O)C3)n[nH]2)C1]

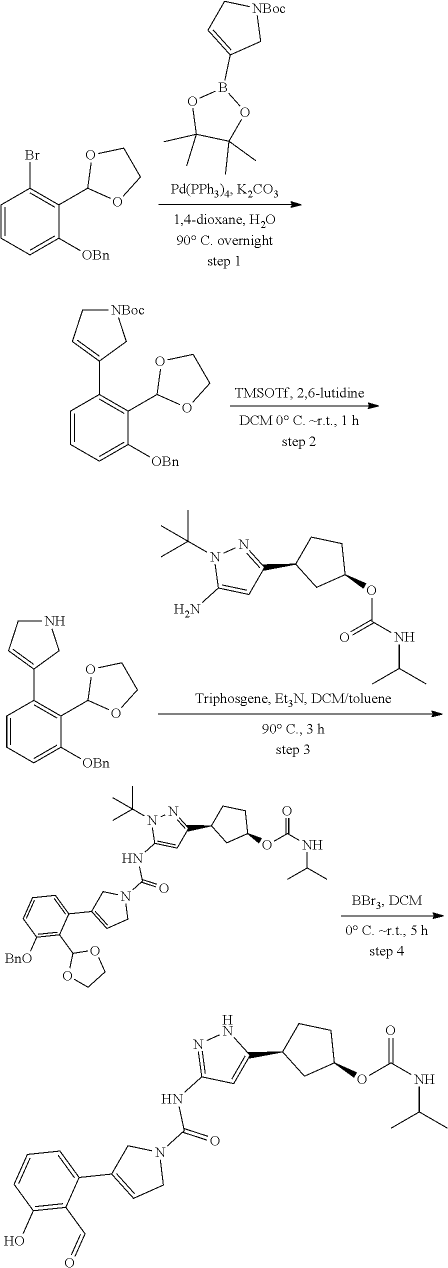

### Step 1: tert-butyl 3-(3-(benzyloxy)-2-(1,3-dioxolan-2-yl)phenyl)-2,5-dihydro-1H-pyrrole-1-carboxylate

Tert-butyl 3-(4,4,5,5-tetramethyl-1,3,2-dioxaborolan-2-yl)-2,5-dihydropyrrole-1-carboxylate (400.3 mg, 1.355 mmol), 2-[2-(benzyloxy)-6-bromophenyl]-1,3-dioxolane (500.2 mg, 1.492 mmol), K₂CO₃ (377.1 mg, 2.708 mmol), tetrakis(triphenylphosphine)palladium(0) (156.0 mg, 0.135 mmol), a stir bar, H₂O (2 mL) and 1,4-dioxane (10 mL) were added to a 40 mL vial. The resulting mixture was maintained under nitrogen and stirred at 90° C. overnight, then quenched with H₂O and extracted with EA. The organic layers were combined, washed with brine, dried over Na₂SO₄, filtered and concentrated. The residue obtained was purified by silica gel chromatography (PE/EA=0-25%) to afford tert-butyl 3-[3-(benzyloxy)-2-(1,3-dioxolan-2-yl)phenyl]-2,5-dihydropyrrole-1-carboxylate (560.0 mg, 97.58%) as yellow oil. MS (ESI) calcd. for C₂₅H₂₉NO₅, 423.20 m/z, found: 368.05 [M−56+H]⁺.

### Step 2: 3-(3-(benzyloxy)-2-(1,3-dioxolan-2-yl)phenyl)-2,5-dihydro-1H-pyrrole

Tert-butyl 3-[3-(benzyloxy)-2-(1,3-dioxolan-2-yl)phenyl]-2,5-dihydropyrrole-1-carboxylate (540.0 mg, 1.275 mmol), a stir bar and DCM (15 mL) were added to a 100 mL flask and stirred until homogenous, then treated with 2,6-lutidine (546.1 mg, 5.095 mmol) and trimethylsilyl triflate (567.3 mg, 2.551 mmol) at 0° C. The resulting mixture was stirred at rt. For 1 h. The reaction was quenched with H₂O and extracted with DCM. The organic layers were combined, washed with brine, dried over Na₂SO₄, filtered and concentrated. The residue obtained was purified by silica gel chromatography (MeOH (0.1% V NH₃·H₂O)/DCM)=0-7%) to afford 3-[3-(benzyloxy)-2-(1,3-dioxolan-2-yl)phenyl]-2,5-dihydro-1H-pyrrole (270.0 mg, 65.48%) as a yellow solid. MS (ESI) calcd. for C₂₀H₂₁NO₃, 323.15, found, 324.10 [M+H]⁺.

### Step 3: (1R,3S)-3-(5-(3-(3-(benzyloxy)-2-(1,3-dioxolan-2-yl)phenyl)-2,5-dihydro-1H-pyrrole-1-carboxamido)-1-(tert-butyl)-1H-pyrazol-3-yl)cyclopentyl isopropylcarbamate

(1R,3S)-3-(5-amino-1-(tert-butyl)-1H-pyrazol-3-yl)cyclopentyl isopropylcarbamate (100.3 mg, 0.324 mmol), triphosgene (125.2 mg, 0.421 mmol), a stir bar, DCM (2 mL) and toluene (2 mL) were added to a 20 mL vial and stirred until homogenous, then treated with triethylamine (330.2 mg, 3.261 mmol) at 0° C. under N₂. The mixture was stirred at 90° C. for 20 min. After cooling down to rt., a mixture of 3-[3-(benzyloxy)-2-(1,3-dioxolan-2-yl)phenyl]-2,5-dihydro-1H-pyrrole (157.1 mg, 0.485 mmol) and toluene (1 mL) were added. The mixture was stirred at 90° C. for 2 h, then quenched with H₂O and extracted with DCM. The organic layers were combined, washed with brine, dried over Na₂SO₄, filtered and concentrated. The residue obtained was purified by reverse phase chromatography (MeCN/H₂O (10 mM NH₄HCO₃)=5˜35%) to afford (1R,3S)-3-(5-(3-(3-(benzyloxy)-2-(1,3-dioxolan-2-yl)phenyl)-2,5-dihydro-1H-pyrrole-1-carboxamido)-1-(tert-butyl)-1H-pyrazol-3-yl)cyclopentyl isopropylcarbamate (100.0 mg, 46.89%) as a yellow solid. MS (ESI) calcd. for C₃₇H₄₇N₅O₆, 657.35, found, 658.30 [M+H]⁺.

### Step 4: (1R,3S)-3-(3-(3-(2-formyl-3-hydroxyphenyl)-2,5-dihydro-1H-pyrrole-1-carboxamido)-1H-pyrazol-5-yl)cyclopentyl isopropylcarbamate

(1R,3S)-3-(5-(3-(3-(benzyloxy)-2-(1,3-dioxolan-2-yl)phenyl)-2,5-dihydro-1H-pyrrole-1-carboxamido)-1-(tert-butyl)-1H-pyrazol-3-yl)cyclopentyl isopropylcarbamate (100.0 mg, 0.152 mmol), a stir bar and DCM (4 mL) were added to a 50 mL flask and stirred until homogenous, then treated with boron tribromide (1.52 mL, 1.520 mmol, 1 M in DCM) at 0° C. The resulting mixture was stirred at rt. For 5 h. The reaction was quenched with H₂O and extracted with DCM. The organic layers were combined, dried over Na₂SO₄, filtered and concentrated. The residue obtained was purified by Prep-HPLC (Column: Xbridge Prep OBD C18 Column, 30×150 mm, 5 μm; Mobile Phase A: Water (0.05% TFA), Mobile Phase B: ACN; Flow rate: 60 mL/min; Gradient: 29% B to 54% B in 10 min; Wave Length: 254/220 nm; RT1 (min): 8.1) to afford (1R,3S)-3-{5-[3-(2-formyl-3-hydroxyphenyl)-2,5-dihydropyrrole-1-carbonylamino]-2H-pyrazol-3-yl}cyclopentyl N-isopropylcarbamate (13.4 mg, 18.46%) as a white solid. MS (ESI) calcd. for C₂₄H₂₉N₅O₅, 467.22, found, 468.25 [M+H]⁺. ¹H NMR (400 MHz, DMSO-d₆) δ 11.65 (s, 1H), 10.19 (s, 1H), 8.90 (s, 1H), 7.59 (t, J=8.0 Hz, 1H), 7.05-6.88 (m, 3H), 6.13 (s, 1H), 6.02 (t, J=2.0 Hz, 1H), 5.00 (s, 1H), 4.55-4.48 (m, 2H), 4.40 (s, 2H), 3.63-3.50 (m, 1H), 3.11-2.96 (m, 1H), 2.49-2.39 (m, 1H), 2.08-1.96 (m, 1H), 1.96-1.83 (m, 1H), 1.79-1.67 (m, 2H), 1.66-1.55 (m, 1H), 1.04 (d, J=6.5 Hz, 6H).

### Example 9.3: Synthesis of Compound 33: (1R,3S)-3-(5-{[(3-formyl-4-hydroxyphenyl) carbamoyl]amino}-2H-pyrazol-3-yl)cyclopentyl N-isopropylcarbamate

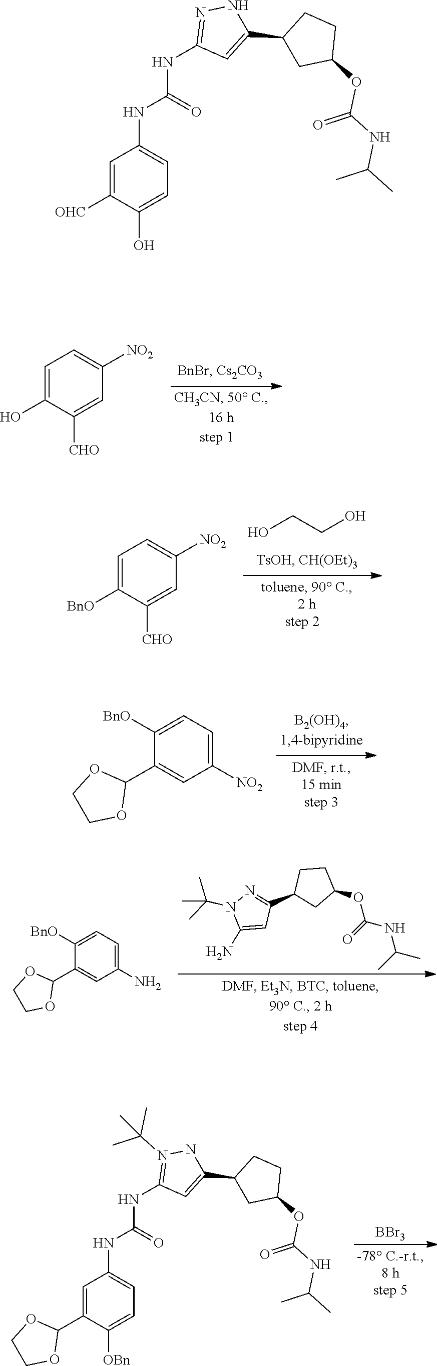

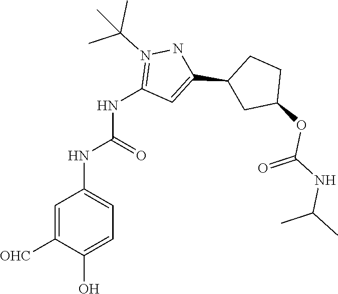

### Step 1: Synthesis of 2-(benzyloxy)-5-nitrobenzaldehyde

5-nitrosalicylaldehyde (4 g, 23.935 mmol), a stir bar, Cs₂CO₃ (1.5 g, 47.870 mmol) and acetonitrile (60 mL) were added to a 250 mL round-bottom flask and stirred until homogeneous, then treated with benzyl bromide (6.1 g, 35.902 mmol). The reaction mixture was stirred at 50° C. overnight and quenched with a saturated solution of NH₄Cl, extracted with EA (250 mL×3). The combined organic layers were washed with brine, dried over anhydrous sodium sulfate, filtered, and concentrated under vacuum. The residue was purified by silica gel chromatography (0-30% PE/EA) to afford 2-(benzyloxy)-5-nitrobenzaldehyde as a yellow solid (5 g, 81.21%). ¹H NMR (400 MHz, DMSO-d₆) δ 10.39 (s, 1H), 8.69-8.28 (m, 2H), 7.58-7.27 (m, 8H).

### Step 2: Synthesis of 2-[2-(benzyloxy)-5-nitrophenyl]-1,3-dioxolane

2-(benzyloxy)-5-nitrobenzaldehyde (5 g, 19.437 mmol), ethylene glycol (6 g, 97.185 mmol), para-toluene sulfonic acid (0.33 g, 1.944 mmol), and toluene (80 mL) were added to a 250 mL round-bottom flask and stirred until homogeneous, then treated with triethyl orthoformate (8.64 g, 58.311 mmol). The reaction mixture was stirred at 90° C. for 2 h and concentrated under vacuum. The residue was purified by silica gel chromatography (0-50% PE/EA) to afford 2-[2-(benzyloxy)-5-nitrophenyl]-1,3-dioxolane as a yellow solid (5.2 g, 88.79%). MS (ESI) mass calcd. for C₁₆H₁₅NO₅, 301.09 m/z, found 302.05 [M+H]⁺.

### Step 3: Synthesis of 4-(benzyloxy)-3-(1,3-dioxolan-2-yl)aniline

2-[2-(benzyloxy)-5-nitrophenyl]-1,3-dioxolane (300 mg, 0.996 mmol), tetrahydroxydiborane (267.7 mg, 2.988 mmol) and dimethylformamide (4 mL) were added to a 20 mL vail and stirred until homogeneous, then treated with bipyridine (15.5 mg, 0.100 mmol) at 0° C. The reaction mixture was stirred at rt. for 15 min. The residue was purified by reverse-phase chromatography (5-60%, ACN/10 mM NH₄HCO₃ water) to afford 4-(benzyloxy)-3-(1,3-dioxolan-2-yl)aniline (100 mg, 37.02%) as a white solid. MS (ESI) mass calcd. for C₁₆H₁₇NO₃, 271.12 m/z, found 272.10 [M+H]⁺.

### Step 4: Synthesis of (1R,3S)-3-[5-({[4-(benzyloxy)-3-(1,3-dioxolan-2-yl)phenyl]carbamoyl}amino)-2-tert-butylpyrazol-3-yl]cyclopentyl N-isopropylcarbamate

A solution of (1R,3S)-3-(5-amino-1-(tert-butyl)-1H-pyrazol-3-yl)cyclopentyl isopropylcarbamate (100 mg, 0.324 mmol) in DCM (11 mL) and toluene (6 mL) was treated BTC (125 mg, 0.421 mmol) for 1 h at room temperature under nitrogen atmosphere, followed by the addition of triethylamine (164 mg, 1.620 mmol) dropwise at 0° C. The reaction mixture was heated to 85° C. stirred for 20 min, add 4-(benzyloxy)-3-(1,3-dioxolan-2-yl)aniline (100 mg, 0.400 mmol) in toluene (1.78 mL) was added and let stir for 1 h at 90° C. The reaction was quenched with H₂O and extracted with EA. The combined organic layers were washed with brine, dried over anhydrous sodium sulfate, filtered and concentrated under vacuum. The residue was purified by reverse-phase chromatography (5%-60%, ACN/10 mM NH₄HCO₃ water) to afford (1R,3S)-3-(5-(3-(4-(benzyloxy)-3-(1,3-dioxolan-2-yl)phenyl)ureido)-1-(tert-butyl)-1H-pyrazol-3-yl)cyclopentyl isopropylcarbamate (60 mg, 30.55%) as a white solid. MS (ESI) mass calcd. for C₃₃H₄₃N₅O₆, 605.32 m/z, found 606.20 [M+H]⁺.

### Step 5: Synthesis of (1R,3S)-3-(5-{[(3-formyl-4-hydroxyphenyl)carbamoyl]amino}-2H-pyrazol-3-yl)cyclopentyl N-isopropylcarbamate

(1R,3S)-3-[5-({[4-(benzyloxy)-3-(1,3-dioxolan-2-yl)phenyl]carbamoyl}amino)-2-tert butylpyrazol-3-yl]cyclopentyl N-isopropylcarbamate (25 mg, 0.041 mmol) and DCM (5 mL) were added to a 100 mL round-bottom flask and stirred until homogeneous. The resulting mixture was stirred under N₂ atmosphere. The resulting mixture was stirred at −70° C., then treated with boron tribromide (0.12 mL, 1 M). The resulting mixture was stirred at −70° C. for 1 h, then stirred at rt. for 3 h. The reaction was quenched with H₂O and extracted with EA. The combined organic layers were washed with brine, dried over anhydrous sodium sulfate, filtered and concentrated under vacuum. The residue was purified by Prep-HPLC (Column: Xselect CSH C18 OBD Column, 30×150 mm, 5 μm; Mobile Phase A: Water (0.05% TFA), Mobile Phase B: ACN; Flow rate: 60 mL/min; Gradient: 18% B to 48% B in 7 min; Wave Length: 254/220 nm; RT1 (min): 6.1) to afford (1R,3S)-3-(5-{[(3-formyl-4-hydroxyphenyl)carbamoyl]amino}-2H-pyrazol-3-yl)cyclopentyl N-isopropylcarbamate as a yellow Solid (1 mg, 5.63%). MS (ESI) mass calcd. for C₂₀H₂₅N₅O₅, 415.18 m/z, found 416.20 [M+H]⁺. ¹H NMR (400 MHz, Methanol-d₄) δ 7.40 (d, J=2.7 Hz, 1H), 7.32 (dd, J=8.7, 2.7 Hz, 1H), 6.82-6.77 (m, 1H), 6.10-6.04 (m, 1H), 5.63-5.59 (m, 1H), 3.76-3.66 (m, 2H), 3.20-3.15 (m, 1H), 2.56-2.50 (m, 1H), 2.21-2.15 (m, 1H), 2.03-1.82 (m, 5H), 1.41-1.36 (m, 1H), 1.22-1.05 (m, 7H).

### Example 9.4: Synthesis of Compound 35: 2-(2-formyl-3-hydroxyphenyl)ethyl N-{5-[(1S,3R)-3-[(isopropylcarbamoyl)oxy]cyclopentyl]-1H-pyrazol-3-yl}carbamate; trifluoroacetic acid

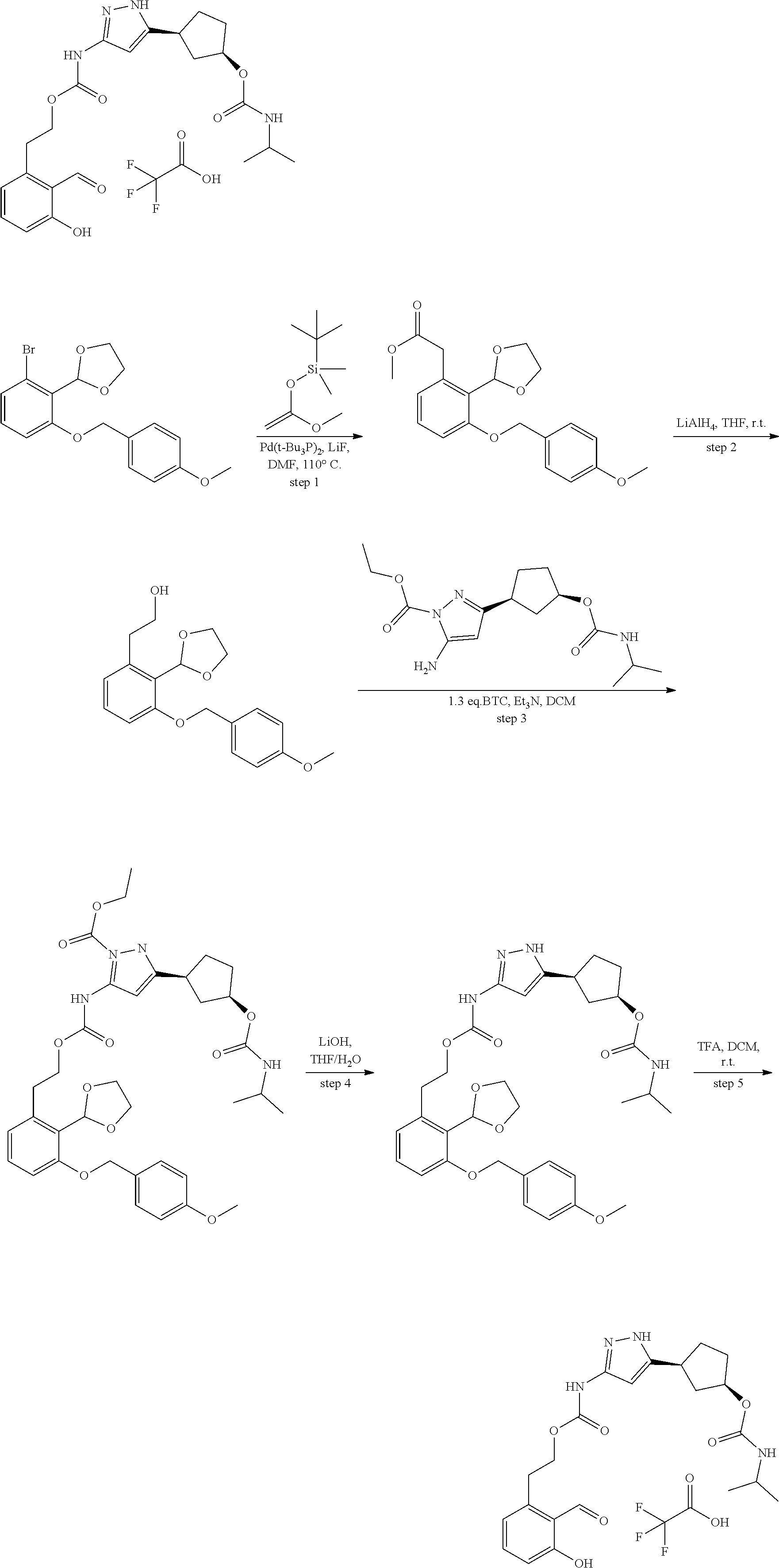

### Step 1: Synthesis of methyl 2-[2-(1,3-dioxolan-2-yl)-3-[(4-methoxyphenyl)methoxy]phenyl]acetate

To a stirred mixture of 2-{2-bromo-6-[(4-methoxyphenyl)methoxy]phenyl}-1,3-dioxolane (2.0 g, 5.476 mmol, 1 equiv), LiF (0.36 g, 13.690 mmol, 2.5 equiv) and Pd(t-Bu₃P)₂ (0.56 g, 1.095 mmol, 0.2 equiv) in DMF (50 mL) were added tert-butyl[(1-methoxyethenyl)oxy]dimethylsilane (5.16 g, 27.380 mmol, 5.0 equiv) dropwise at room temperature under N₂ atmosphere. The resulting mixture was stirred for 1 h at 110° C. under N₂ atmosphere, then cooled to room temperature and diluted with water. The resulting mixture was extracted with EA (3×150 mL). The combined organic layers were washed with brine (3×150 mL), dried over anhydrous Na₂SO₄. After filtration, the filtrate was concentrated under reduced pressure. The residue was purified by silica gel column chromatography, eluted with PE/EA (30%) to afford methyl 2-[2-(1,3-dioxolan-2-yl)-3-[(4-methoxyphenyl)methoxy]phenyl]acetate (1.03 g, 52.48%) as a brown solid. MS (ESI) calcd. for C₂₀H₂₂O₆, 358.14 m/z, found 359.15 [M+H]⁺.

### Step 2: Synthesis of 2-[2-(1,3-dioxolan-2-yl)-3-[(4-methoxyphenyl)methoxy]phenyl]ethanol

To a stirred solution of methyl 2-[2-(1,3-dioxolan-2-yl)-3-[(4-methoxyphenyl)methoxy]phenyl]acetate (500.0 mg, 1.395 mmol, 1 equiv) in THF (8 mL) was added LiAlH₄ (1.12 mL, 2.790 mmol, 2 equiv) dropwise at 0° C. under N₂ atmosphere. The resulting mixture was stirred for 1 h at room temperature under N₂ atmosphere. The reaction was quenched by the addition of Na₂SO₄·10H₂O (1 g) at room temperature. The resulting mixture was filtered, the filter cake was washed with DCM (3×20 mL). The filtrate was concentrated under reduced pressure to afford 2-[2-(1,3-dioxolan-2-yl)-3-[(4-methoxyphenyl)methoxy]phenyl]ethanol (320.0 mg, 69.43%) as a brown semi-solid.

### Step 3: Synthesis of ethyl 5-[({2-[2-(1,3-dioxolan-2-yl)-3-[(4-methoxyphenyl)methoxy]phenyl]ethoxy}carbonyl)amino]-3-[(1S,3R)-3-[(isopropylcarbamoyl)oxy]cyclopentyl]pyrazole-1-carboxylate

To a stirred solution of ethyl 5-amino-3-[(1S,3R)-3-[(isopropylcarbamoyl)oxy]cyclopentyl]pyrazole-1-carboxylate (250.0 mg, 0.771 mmol, 1.00 equiv) and triphosgene (bis(trichloromethyl) carbonate (297.3 mg, 1.002 mmol, 1.3 equiv) in DCM (2 mL) and toluene (6 mL) were added TEA (1.07 mL, 7.710 mmol, 10 equiv) dropwise at 0° C. under N₂ atmosphere. The resulting mixture was stirred for 0.5 h at 90° C. under N₂ atmosphere. To the above mixture was added 2-[2-(1,3-dioxolan-2-yl)-3-[(4-methoxyphenyl)methoxy]phenyl]ethanol (254.6 mg, 0.771 mmol, 1 equiv) in toluene (4 mL) dropwise at room temperature. The resulting mixture was stirred for additional 1 h at 90° C., then cooled to room temperature and diluted with water. The resulting mixture was extracted with DCM (3×30 mL). The combined organic layers were washed with brine (3×30 mL). After filtration, the filtrate was concentrated under reduced pressure. The residue was purified by reversed-phase flash chromatography with the following conditions: column, C18 silica gel; mobile phase, H₂O (0.05% TFA Modifier) in ACN, 10% to 70% gradient in 50 min; detector, UV 254 nm. This resulted in ethyl 5-[({2-[2-(1,3-dioxolan-2-yl)-3-[(4-methoxyphenyl)methoxy]phenyl]ethoxy}carbonyl)amino]-3-[(1S,3R)-3-[(isopropylcarbamoyl) oxy]cyclopentyl]pyrazole-1-carboxylate (260.0 mg, 49.56%) as a yellow oil. MS (ESI) calcd. for C₃₅H₄₄N₄O₁₀, 680.31 m/z, found 681.40 [M+H]⁺.

### Step 4: Synthesis of 2-[2-(1,3-dioxolan-2-yl)-3-[(4-methoxyphenyl)methoxy]phenyl]ethyl N-{5-[(1S,3R)-3-[(isopropylcarbamoyl)oxy]cyclopentyl]-1H-pyrazol-3-yl}carbamate

To a stirred solution of ethyl 5-[({2-[2-(1,3-dioxolan-2-yl)-3-[(4-methoxyphenyl)methoxy]phenyl]ethoxy}carbonyl)amino]-3-[(1S,3R)-3-[(isopropylcarbamoyl)oxy]cyclopentyl]pyrazole-1-carboxylate (100.0 mg, 0.147 mmol, 1 equiv) in THF (2 mL) and H₂O (2 mL) was added LiOH (17.6 mg, 0.735 mmol, 5 equiv) at room temperature. The resulting mixture was stirred for 1 h at room temperature. The mixture acidified to pH ˜5 with HCl (2 M). The aqueous layer was extracted with EA (3×20 mL). The resulting mixture was concentrated under reduced pressure. This resulted in 2-[2-(1,3-dioxolan-2-yl)-3-[(4-methoxyphenyl)methoxy]phenyl]ethyl N-{5-[(1S,3R)-3-[(isopropylcarbamoyl)oxy]cyclopentyl]-1H-pyrazol-3-yl}carbamate (80.0 mg, 89.47%) as a yellow oil. MS (ESI) calcd. for C₃₂H₄₀N₄O₈, 608.28 m/z, found 609.30 [M+H]⁺.

### Step 5: Synthesis of 2-(2-formyl-3-hydroxyphenyl)ethyl N-{5-[(1S,3R)-3-[(isopropyl carbamoyl)oxy]cyclopentyl]-1H-pyrazol-3-yl}carbamate; trifluoroacetic acid

To a stirred solution of 2-[2-(1,3-dioxolan-2-yl)-3-[(4-methoxyphenyl)methoxy]phenyl]ethyl N-{5-[(1S,3R)-3-[(isopropylcarbamoyl)oxy]cyclopentyl]-1H-pyrazol-3-yl}carbamate (80.0 mg, 0.131 mmol, 1 equiv) in DCM (1 mL, 15.731 mmol) was added TFA (4 mL, 53.852 mmol) dropwise at room temperature under. The resulting mixture was stirred for 1 h at room temperature. The resulting mixture was concentrated under reduced pressure. The crude product was purified by Prep-HPLC with the following conditions (Column: Xselect CSH OBD Column, 30×150 mm, 5 um; Mobile Phase A: Water (0.05% TFA), Mobile Phase B: ACN; Flow rate: 60 mL/min; Gradient: 27% B to 49% B in 10 min; Wave Length: 254/220 nm; RT1 (min): 9.5) to afford 2-(2-formyl-3-hydroxyphenyl)ethyl N-{5-[(1S,3R)-3-[(isopropylcarbamoyl)oxy]cyclopentyl]-1H-pyrazol-3-yl}carbamate; trifluoroacetic acid (9.5 mg, 12.33%) as a white solid. MS (ESI) calcd. for C₂₄H₂₉F₃N₄O₈, 444.20 m/z, found 445.20 [M+H]⁺. ¹H NMR (400 MHz, DMSO-d₆) δ (ppm): 11.27 (br s, 1H), 10.44 (s, 1H), 9.85 (br s, 1H), 7.43-7.47 (m, 1H), 6.94-6.97 (m, 1H), 6.87-6.89 (m, 1H), 6.82-6.84 (m, 1H), 6.05-6.06 (m, 1H), 4.98-4.99 (m, 1H), 4.21-4.24 (m, 2H), 3.55-3.60 (m, 1H), 3.23-3.26 (m, 2H), 3.00-3.04 (m, 1H), 2.41-2.46 (m, 1H), 1.96-2.00 (m, 1H), 1.85-1.90 (m, 1H), 1.64-1.72 (m, 2H), 1.55-1.61 (m, 1H), 1.02-1.04 (m, 6H). ¹⁹F NMR (376 MHz, DMSO-d₆) δ (ppm): −74.93.

### Example 10: Synthesis of Compound 34: (1R,3S)-3-{5-[3-(2-formyl-3-hydroxyphenoxy) propanamido]-2H-pyrazol-3-yl}cyclopentyl N-isopropylcarbamate; trifluoroacetic acid

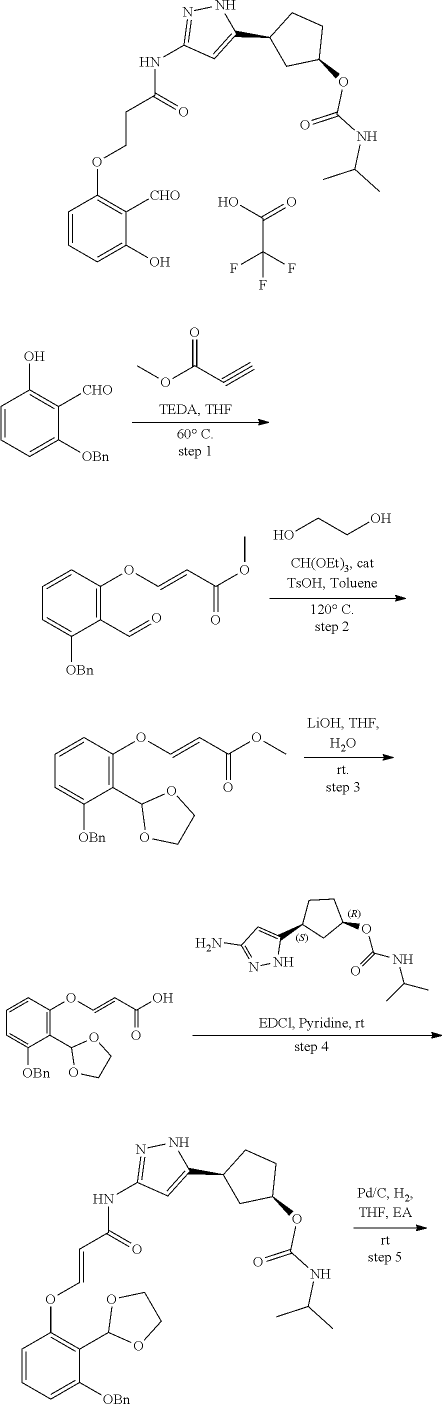

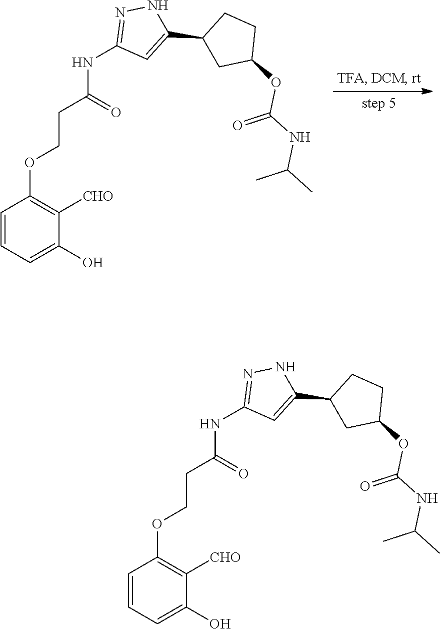 [SMILES: CC(C)NC(=O)O[C@@H]1CC[C@H](c2cc(NC(=O)CCOc3cccc(O)c3C=O)n[nH]2)C1 ; CC(C)NC(=O)O[C@@H]1CC[C@H](c2cc(NC(=O)CCOc3cccc(O)c3C=O)n[nH]2)C1]

### Step 1: Synthesis of methyl (2E)-3-[3-(benzyloxy)-2-formylphenoxy]prop-2-enoate

To a stirred solution of 2-(benzyloxy)-6-hydroxybenzaldehyde (6.0 g, 26.287 mmol, 1.1 equiv) and methyl propiolate (2.01 g, 23.897 mmol, 1 equiv) in THF (80 mL) were added TEDA (0.27 g, 2.390 mmol, 0.1 equiv) at 25° C. The resulting mixture was stirred for 6 h at 60° C. The reaction was monitored by LCMS. The resulting mixture was extracted with EA (3×100 mL). The combined organic layers were washed with brine (2×80 mL), dried over anhydrous Na₂SO₄. After filtration, the filtrate was concentrated under reduced pressure. The residue was purified by silica gel column chromatography, eluted with PE/EA (4/1) to afford methyl (2E)-3-[3-(benzyloxy)-2-formylphenoxy]prop-2-enoate (3.6 g, 35.06%) as yellow solid. MS (ESI) calcd. for C₁₈H₁₆O₅, 312.32 m/z, found 313.05 [M+H]⁺.

### Step 2: Synthesis of methyl (2E)-3-[3-(benzyloxy)-2-(1,3-dioxolan-2-yl)phenoxy]prop-2-enoate

To a stirred solution of methyl (2E)-3-[3-(benzyloxy)-2-formylphenoxy]prop-2-enoate (3.5 g, 11.206 mmol, 1 equiv) and ethylene glycol (3.12 mL, 56.030 mmol, 5 equiv) in Toluene (90 mL) was added (diethoxymethoxy)ethane (5.60 mL, 33.618 mmol, 3 equiv) TsOH (0.19 g, 1.121 mmol, 0.1 equiv) at 25° C. The resulting mixture was stirred for 6 h at 120° C. The reaction was monitored by LCMS. The resulting mixture was extracted with EA (3×100 mL). The combined organic layers were washed with brine (2×80 mL), dried over anhydrous Na₂SO₄. After filtration, the filtrate was concentrated under reduced pressure. The residue was purified by silica gel column chromatography, eluted with PE/EA (5/1) to afford methyl (2E)-3-[3-(benzyloxy)-2-(1,3-dioxolan-2-yl)phenoxy]prop-2-enoate (3.0 g, 73.84%) as a yellow solid. MS (ESI) calcd. for C₂₀H₂₀O₆, 356.37 m/z, found 357.15 [M+H]⁺.

### Step 3: Synthesis of (2E)-3-[3-(benzyloxy)-2-(1,3-dioxolan-2-yl)phenoxy]prop-2-enoic acid

To a stirred solution of methyl (2E)-3-[3-(benzyloxy)-2-formylphenoxy]prop-2-enoate (0.8 g, 2.561 mmol, 1 equiv) in THF (12 mL) and H₂O (12 mL) was added LiOH (0.31 g, 12.805 mmol, 5 equiv) at 25° C. The resulting mixture was stirred for 12 h at 25° C. The mixture was acidified to pH 7 with HCl (1.0 1M). The resulting mixture was extracted with EA (3×60 mL). The combined organic layers were washed with brine (2×50 mL), dried over anhydrous Na₂SO₄. After filtration, the filtrate was concentrated under reduced pressure. This resulted in (2E)-3-[3-(benzyloxy)-2-(1,3-dioxolan-2-yl)phenoxy]prop-2-enoic acid (0.7 g, 77.11%) as a white solid. MS (ESI) calcd. for C₁₉H₁₈O₆, 342.35 m/z, found 341.05 [M−H]⁻.

### Step 4: Synthesis of (1R,3S)-3-{5-[(2E)-3-[3-(benzyloxy)-2-(1,3-dioxolan-2-yl)phenoxy]prop-2-enamido]-2H-pyrazol-3-yl}cyclopentyl N-isopropylcarbamate

To a stirred solution of (2E)-3-[3-(benzyloxy)-2-(1,3-dioxolan-2-yl)phenoxy]prop-2-enoic acid (180.0 mg, 0.526 mmol, 1 equiv) and (1R,3S)-3-(5-amino-2H-pyrazol-3-yl)cyclopentyl N-isopropylcarbamate (132.66 mg, 0.526 mmol, 1 equiv) in pyridine (6 mL) was added EDCI (151.19 mg, 0.789 mmol, 1.5 equiv) at 25° C. The resulting mixture was stirred for 2 h at 25° C. The reaction was monitored by LCMS. The resulting mixture was extracted with EA (3×20 mL). The combined organic layers were washed with brine (2×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O in ACN, 55% gradient in 10 min; detector, UV 254 nm. This resulted in (1R,3S)-3-{5-[(2E)-3-[3-(benzyloxy)-2-(1,3-dioxolan-2-yl)phenoxy]prop-2-enamido]-2H-pyrazol-3-yl}cyclopentyl N-isopropylcarbamate (150.0 mg, 46.51%) as a white solid. MS (ESI) calcd. for C₃₁H₃₆N₄O₇, 576.65 m/z, found 577.30 [M+H]⁺.

### Step 5: Synthesis of (1R,3S)-3-(5-{3-[2-(1,3-dioxolan-2-yl)-3-hydroxyphenoxy]propanamido}-2H-pyrazol-3-yl)cyclopentyl N-isopropylcarbamate

To a stirred solution of (1R,3S)-3-(5-{3-[2-(1,3-dioxolan-2-yl)-3-hydroxyphenoxy]propanamido}-2H-pyrazol-3-yl)cyclopentyl N-isopropylcarbamate (90.0 mg, 34.03%) in THF (5 mL) and EA (5 mL) was added 10% Pd/C (132.88 mg) at 25° C. under H₂ atmosphere. The resulting mixture was stirred for 3 h at 25° C. The reaction was monitored by LCMS. The resulting mixture was filtered, the filter cake was washed with DCM (3×30 mL). The filtrate was concentrated under reduced pressure. This resulted in (1R,3S)-3-(5-{3-[2-(1,3-dioxolan-2-yl)-3-hydroxyphenoxy]propanamido}-2H-pyrazol-3-yl)cyclopentyl N-isopropylcarbamate (90.0 mg, 34.03%) as a yellow solid. MS (ESI) calcd. for C₂₄H₃₂N₄O₇, 488.54 m/z, found 489.30 [M+H]⁺.

### Step 6: Synthesis of (1R,3S)-3-{5-[3-(2-formyl-3-hydroxyphenoxy)propanamido]-2H-pyrazol-3-yl}cyclopentyl N-isopropylcarbamate; trifluoroacetic acid

To a stirred solution of (1R,3S)-3-{5-[(2E)-3-[3-(benzyloxy)-2-(1,3-dioxolan-2-yl)phenoxy]prop-2-enamido]-2H-pyrazol-3-yl}cyclopentyl N-isopropylcarbamate (60.0 mg, 0.104 mmol, 1 equiv) in DCM (3 mL) were added TFA (1 mL) dropwise at 25° C. The resulting mixture was stirred for 1 h at 25° C. The reaction was monitored by LCMS. The resulting mixture was concentrated under reduced pressure. The crude product was purified by reverse phase flash with the following conditions (Column: XBridge Shield RP18 OBD Column, 30×150 mm, 5 um; Mobile Phase A: Water (0.1% TFA), Mobile Phase B: ACN; Flow rate: 60 mL/min; Gradient: 27% B to 53% B in 10 min; Wave Length: 254/220 nm; RT1 (min): 7.42) to afford (1R,3S)-3-{5-[3-(2-formyl-3-hydroxyphenoxy)propanamido]-2H-pyrazol-3-yl}cyclopentyl N-isopropyl carbamate; trifluoroacetic acid (5.4 mg, 9.23%) as a white solid. MS (ESI) calcd. For C₂₄H₂₉F₃N₄O₈, 444.49 m/z, found 445.30 [M+H]⁺. ¹H NMR (300 MHz, DMSO-d6) δ 11.67 (br s, 1H), 10.50 (s, 1H), 10.22 (s, 1H), 7.50-7.56 (m, 1H), 6.93-6.96 (m, 1H), 6.67-6.64 (m, 1H), 6.50-6.53 (m, 1H), 6.30 (s, 1H), 4.98-5.01 (m, 1H), 4.38-4.42 (m, 2H), 3.51-3.59 (m, 1H), 3.00-3.06 (m, 1H), 2.77-2.81 (m, 2H), 2.49-2.51 (m, 1H), 1.91-2.01 (m, 1H), 1.85-1.90 (m, 1H), 1.54-1.71 (m, 3H), 1.00-1.02 (m, 6H). ¹⁹F NMR (282 MHz, DMSO-d₆) δ −74.67.

### Example 11: Synthesis of Compound 20 (1R,3S)-3-(5-{5-[(2-formyl-3-hydroxyphenyl)carbamoyl]-2-methylpyrazole-3-amido}-2H-pyrazol-3-yl)cyclopentyl N-isopropylcarbamate; trifluoroacetic acid

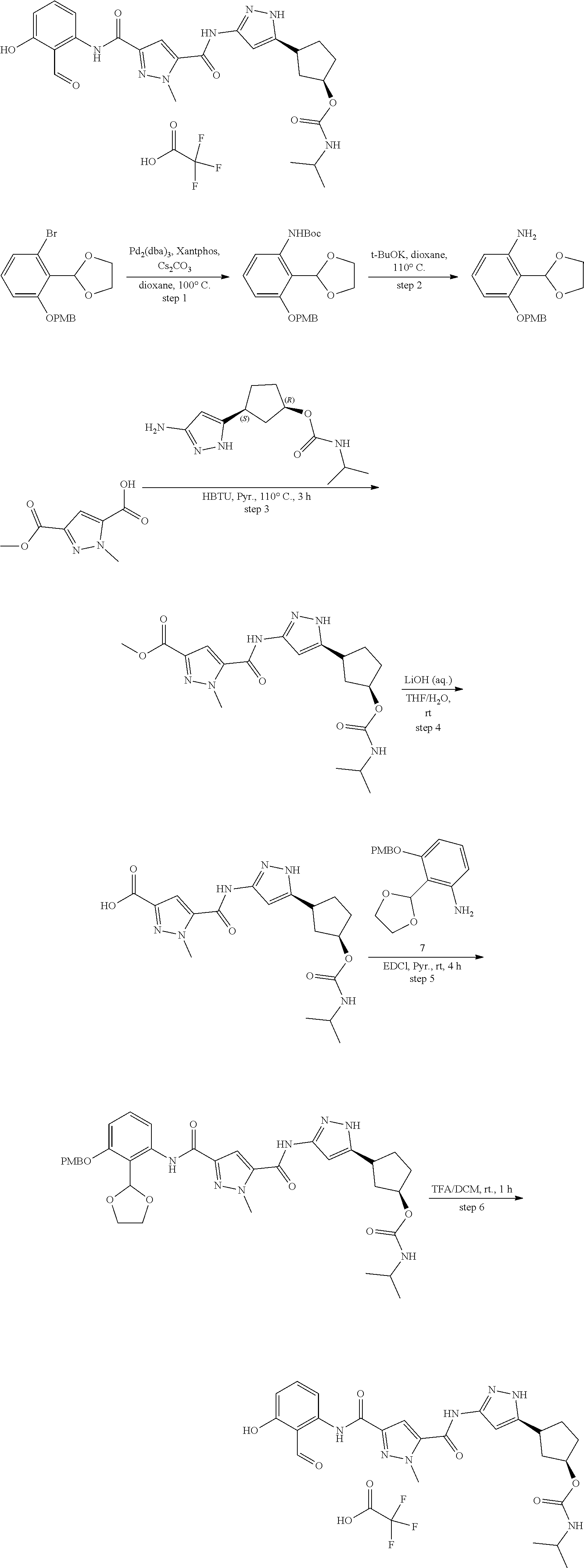

### Step 1: Synthesis of tert-butyl (2-(1,3-dioxolan-2-yl)-3-((4-methoxybenzyl)oxy)phenyl) carbamate

To a stirred solution of 2-{2-bromo-6-[(4-methoxyphenyl)methoxy]phenyl}-1,3-dioxolane (3 g, 8.214 mmol, 1 equiv) and tert-butyl carbamate (1.15 g, 9.857 mmol, 1.2 equiv) in 1,4-dioxane (60 mL) was added Pd₂(dba)₃ (0.75 g, 0.821 mmol, 0.1 equiv), Xantphos (0.95 g, 1.643 mmol, 0.2 equiv) and Cs₂CO₃ (8.03 g, 24.642 mmol, 3 equiv) in portions at room temperature under N₂ atmosphere. The resulting mixture was stirred for 2 h at 100° C. under N₂ atmosphere. The reaction mixture was then treated with H₂O (100 mL), dropwise over 10 min, extracted with EtOAc (100 mL×2), and the combined extracts washed with brine (100 mL), dried over anhydrous Na₂SO₄, filtered, and concentrated to dryness in vacuo to give a nearly brown viscous oil. The oil was then subjected to silica gel chromatography (0-30% EtOAc/petroleum ether) to give tert-butyl N-[2-(1,3-dioxolan-2-yl)-3-[(4-methoxyphenyl)methoxy]phenyl]carbamate (4.2 g, 82.27%) as a yellow solid. MS (ESI) calcd. for C₂₂H₂₇NO₆, 401.18 m/z, found 402.15 [M+H]⁺.

### Step 2: Synthesis of 2-(1,3-dioxolan-2-yl)-3-((4-methoxybenzyl)oxy)aniline

To a stirred solution of tert-butyl N-[2-(1,3-dioxolan-2-yl)-3-[(4-methoxyphenyl)methoxy]phenyl]carbamate (910.0 mg, 2.267 mmol, 1 equiv) in 1,4-dioxane (10 mL) was added t-BuOK (508.7 mg, 4.534 mmol, 2 equiv) in portions at room temperature. The resulting mixture was stirred for 2 h at 80° C. The reaction mixture was then treated with H₂O (50 mL), dropwise over 10 min, extracted with EtOAc (50 mL×2), and the combined extracts washed with brine (50 mL), dried over anhydrous Na₂SO₄, filtered, and concentrated to dryness in vacuo to give a nearly brown viscous oil. The oil was then subjected to silica gel chromatography (0-30% EtOAc/petroleum ether) to give 2-(1,3-dioxolan-2-yl)-3-[(4-methoxyphenyl)methoxy]aniline (130.0 mg, 17.68%) as a white solid. MS (ESI) calcd. for C₁₇H₁₉NO₄, 301.13 m/z, found 302.15 [M+H]⁺.

### Step 3: Synthesis of methyl 5-((5-((1S,3R)-3-((isopropylcarbamoyl)oxy)cyclopentyl)-1H-pyrazol-3-yl)carbamoyl)-1-methyl-1H-pyrazole-3-carboxylate

To a stirred solution of 5-(methoxycarbonyl)-2-methylpyrazole-3-carboxylic acid (364.9 mg, 1.982 mmol, 1 equiv) and (1R,3S)-3-(5-amino-2H-pyrazol-3-yl)cyclopentyl N-isopropyl-carbamate (500.0 mg, 1.982 mmol, 1 equiv) in pyridine (15 mL) were added HBTU (901.8 mg, 2.378 mmol, 1.2 equiv) in portions at room temperature. The resulting mixture was stirred for 1 h at room temperature. The reaction mixture was then treated with H₂O (30 mL), dropwise over 10 min, extracted with EtOAc (30 mL×2), and the combined extracts washed with brine (10 mL), dried over anhydrous Na₂SO₄, filtered, and concentrated to dryness in vacuo to give a nearly yellow viscous oil. The oil was then subjected to silica gel chromatography (0-10% EtOAc/petroleum ether) to give methyl 5-({5-[(1S,3R)-3-[(isopropylcarbamoyl)oxy]cyclopentyl]-1H-pyrazol-3-yl}carbamoyl)-1-methylpyrazole-3-carboxylate (270.0 mg, 32.56%) as a yellow solid. MS (ESI) calcd. for C₁₉H₂₆N₆O₅, 418.20 m/z, found 419.20 [M+H]⁺.

### Step 4: Synthesis of 5-((5-((1S,3R)-3-((isopropylcarbamoyl)oxy)cyclopentyl)-1H-pyrazol-3-yl)carbamoyl)-1-methyl-1H-pyrazole-3-carboxylic acid

To a stirred solution of methyl 5-({5-[(1S,3R)-3-[(isopropylcarbamoyl)oxy]cyclopentyl]-1H-pyrazol-3-yl}carbamoyl)-1-methylpyrazole-3-carboxylate (200.0 mg, 0.478 mmol, 1 equiv) and in THF (4 mL) and H₂O (4 mL) was added LiOH (57.2 mg, 2.390 mmol, 5 equiv) at room temperature. The resulting mixture was stirred for 1 h at room temperature. The mixture was acidified to pH ˜5 with HCl (2 M). The reaction mixture was then treated with H₂O (30 mL), dropwise over 10 min, extracted with EtOAc (10 mL×2), and the combined extracts washed with brine (10 mL), dried over anhydrous Na₂SO₄, filtered, and concentrated to dryness in vacuo to give 5-({5-[(1S,3R)-3-[(isopropylcarbamoyl)oxy]cyclopentyl]-1H-pyrazol-3-yl}carbamoyl)-1-methylpyrazole-3-carboxylic acid (110.0 mg, 54.84%) as a yellow solid. MS (ESI) calcd. for C₁₈H₂₄N₆O₅, 404.18 m/z, found 403.10 [M−H]⁻.

### Step 5: Synthesis of (1R,3S)-3-(3-(3-((2-(1,3-dioxolan-2-yl)-3-((4-methoxybenzyl)oxy)phenyl) carbamoyl)-1-methyl-1H-pyrazole-5-carboxamido)-1H-pyrazol-5-yl)cyclopentyl isopropyl-carbamate

To a stirred solution of 5-({5-[(1S,3R)-3-[(isopropylcarbamoyl)oxy]cyclopentyl]-1H-pyrazol-3-yl}carbamoyl)-1-methylpyrazole-3-carboxylic acid (209.4 mg, 0.517 mmol, 1.2 equiv) and 2-(1,3-dioxolan-2-yl)-3-[(4-methoxyphenyl)methoxy]aniline (130.0 mg, 0.431 mmol, 1 equiv) in pyridine (8 mL) was added EDCI (165.4 mg, 0.862 mmol, 2 equiv) in portions at room temperature. The resulting mixture was stirred for 1 h at room temperature. The reaction mixture was then treated with H₂O (10 mL), dropwise over 10 min, extracted with EtOAc (10 mL×2), and the combined extracts washed with brine (10 mL), dried over anhydrous Na₂SO₄, filtered, and concentrated to dryness in vacuo to give a nearly yellow viscous oil. The oil was then subjected to silica gel chromatography (0-10% DCM/MeOH) to give (1R,3S)-3-(3-(3-((2-(1,3-dioxolan-2-yl)-3-((4-methoxybenzyl)oxy)phenyl)carbamoyl)-1-methyl-1H-pyrazole-5-carboxamido)-1H-pyrazol-5-yl)cyclopentyl isopropylcarbamate (60.0 mg, 17.18%) as a white solid. MS (ESI) calcd. for C₃₅H₄₁N₇O₈, 687.30 m/z, found 688.35 [M+H]⁺.

### Step 6: Synthesis of (1R,3S)-3-(5-{5-[(2-formyl-3-hydroxyphenyl)carbamoyl]-2-methyl pyrazole-3-amido}-2H-pyrazol-3-yl)cyclopentyl N-isopropylcarbamate; trifluoroacetic acid

To a stirred solution of (1R,3S)-3-[5-(5-{[2-(1,3-dioxolan-2-yl)-3-[(4-methoxyphenyl)methoxy]phenyl]carbamoyl}-2-methylpyrazole-3-amido)-2H-pyrazol-3-yl]cyclopentyl N-isopropyl-carbamate (60.0 mg, 0.087 mmol, 1 equiv) in DCM (3 mL) was added TFA (0.6 mL) in portions at room temperature. The resulting mixture was stirred for 1 h at room temperature. The residue was diluted with water, then adjusted to pH 6-7 with NaHCO₃, dropwise over 10 min, extracted with DCM (10 mL×2), and the combined extracts washed with brine (10 mL), dried over anhydrous Na₂SO₄, filtered, and concentrated to dryness in vacuo to give a nearly yellow viscous oil. The crude product was then purified by Prep-HPLC with the following conditions: Column: Xselect CSH OBD Column, 30×150 mm, 5 um; Mobile Phase A: Water (0.05% TFA), Mobile Phase B: ACN; Flow rate: 60 mL/min; Gradient: 29% B to 51% B in 10 min; Wave Length: 254 nm/220 nm; RT1 (min): 9) to afford (1R,3S)-3-(5-{5-[(2-formyl-3-hydroxyphenyl)carbamoyl]-2-methylpyrazole-3-amido}-2H-pyrazol-3-yl)cyclopentyl N-isopropylcarbamate; trifluoroacetic acid (3.8 mg, 6.62%) as a light yellow solid. MS (ESI) calcd. for C₂₇H₃₀F₃N₇O₈, 523.22 m/z, found 524.25 [M+H]⁺. ¹H NMR (300 MHz, DMSO-d₆) δ 12.58 (s, 1H), 10.97 (s, 1H), 10.93 (s, 1H), 10.47 (s, 1H), 8.17 (d, J=8.4 Hz, 1H), 7.69 (s, 1H), 7.50 (t, J=8.4 Hz, 1H), 6.92-6.95 (m, 1H), 6.70-6.73 (m, 1H), 6.44 (s, 1H), 4.99-5.05 (m, 1H), 4.22 (s, 3H), 3.55-3.62 (m, 1H), 3.07-3.12 (m, 1H), 2.47-2.49 (m, 1H), 2.00-2.07 (m, 1H), 1.86-1.94 (m, 1H), 1.74-1.76 (m, 2H), 1.57-1.68 (m, 1H), 1.04-1.06 (m, 6H). ¹⁹F NMR (282 MHz, DMSO-d₆) δ −74.50.

### Example 12: Synthesis of Compound 21: (1R,3S)-3-(5-{5-[(2-formylphenyl)carbamoyl]-2-methyl pyrazole-3-amido}-2H-pyrazol-3-yl)cyclopentyl N-isopropylcarbamate; trifluoroacetic acid

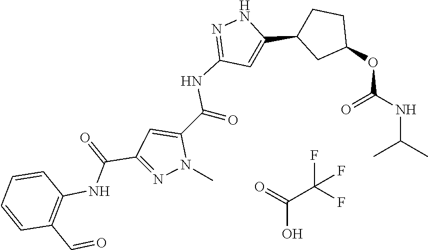

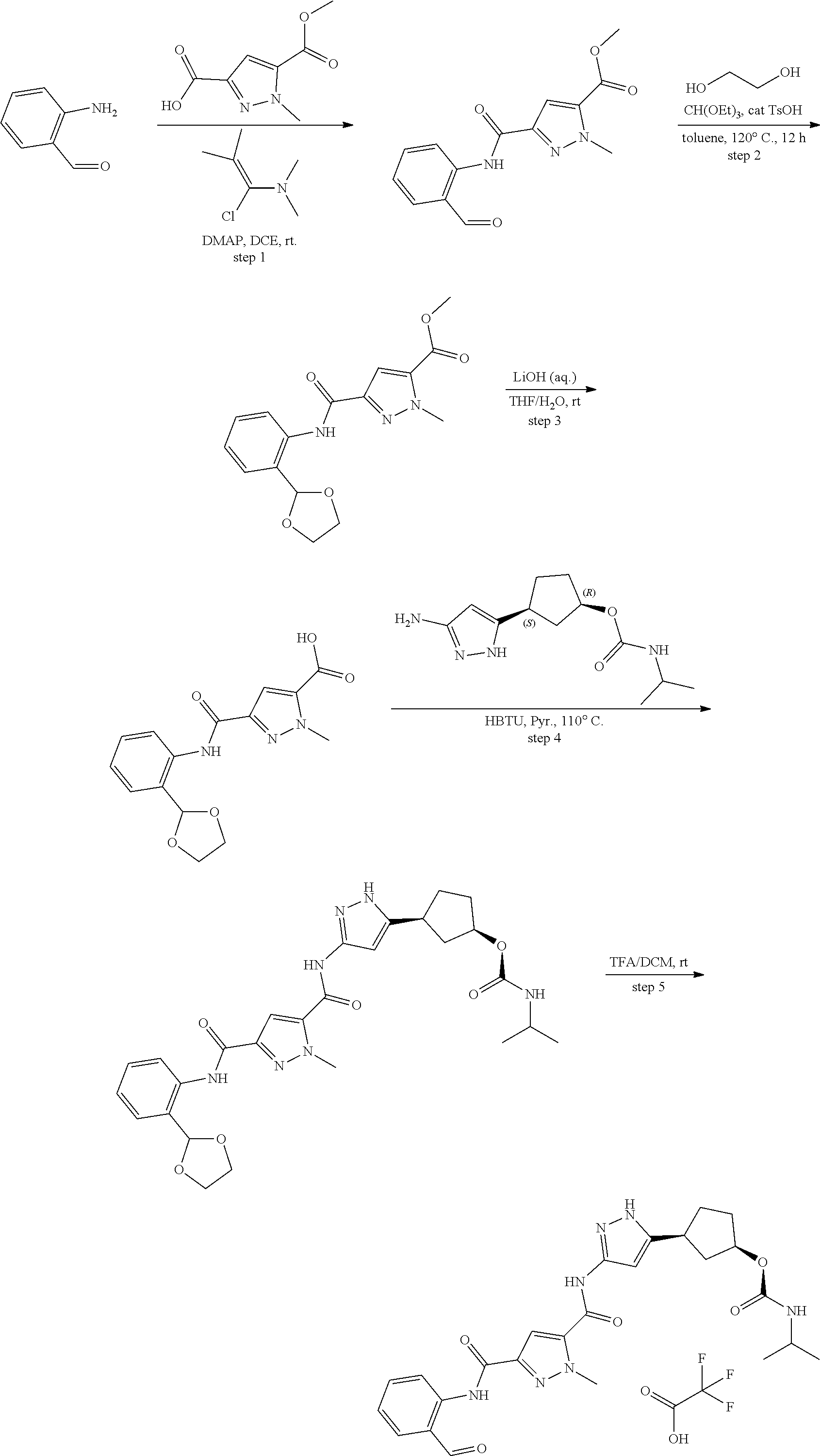

### Step 1: Synthesis of methyl 5-[(2-formylphenyl)carbamoyl]-2-methylpyrazole-3-carboxylate

To a stirred solution of 5-(methoxycarbonyl)-1-methylpyrazole-3-carboxylic acid (986.6 mg, 5.357 mmol, 1.1 equiv) in DCE (5 mL) was added (1-chloro-2-methylprop-1-en-1-yl)dimethylamine (911.1 mg, 6.818 mmol, 1.4 equiv) at room temperature. The resulting mixture was stirred for 12 h. To this solution was added 2-aminobenzaldehyde (590.0 mg, 4.870 mmol, 1 equiv) and DMAP (1785.1 mg, 14.610 mmol, 3 equiv). The resulting mixture was stirred for 10 h at 50° C. then cooled to room temperature and diluted with water. The resulting mixture was extracted with ethyl acetate (3×50 mL). The organic layers were combined, dried over anhydrous sodium sulfate, filtered and concentrated. After filtration, the filtrate was concentrated under reduced pressure. The residue obtained was purified by silica gel chromatography (50% ethyl acetate/petroleum ether) to afford methyl 5-[(2-formylphenyl)carbamoyl]-2-methylpyrazole-3-carboxylate (700.0 mg, 50.02%) as a yellow oil. MS (ESI) calcd. for C₁₄H₁₃N₃O₄, 287.09 m/z, found 288.10 [M+H]⁺.

### Step 2: Synthesis of methyl 5-{[2-(1,3-dioxolan-2-yl)phenyl]carbamoyl}-2-methylpyrazole-3-carboxylate

To a stirred solution of methyl 5-[(2-formylphenyl)carbamoyl]-2-methylpyrazole-3-carboxylate (700.0 mg, 2.437 mmol, 1 equiv) and ethylene glycol (756.2 mg, 12.185 mmol, 5 equiv) in toluene (10 mL) was added Triethyl orthoformate (1083.4 mg, 7.311 mmol, 3 equiv) and TsOH (41.9 mg, 0.244 mmol, 0.1 equiv) at room temperature The resulting mixture was stirred for 12 h at 120° C. then cooled to room temperature and diluted with water. The resulting mixture was extracted with ethyl acetate (3×50 mL). The organic layers were combined, dried over anhydrous sodium sulfate, filtered and concentrated under reduced pressure. The residue obtained was purified by silica gel chromatography (60% ethyl acetate/petroleum ether) to afford methyl 5-{[2-(1,3-dioxolan-2-yl)phenyl]carbamoyl}-2-methylpyrazole-3-carboxylate (530.0 mg, 65.65%) as yellow oil. MS (ESI) calcd. for C₁₆H₁₇N₃O₅, 331.12 m/z, found 332.15 [M+H]⁺.

### Step 3: Synthesis of 5-{[2-(1,3-dioxolan-2-yl)phenyl]carbamoyl}-2-methyl pyrazole-3-carboxylic acid

To a stirred solution of methyl 5-{[2-(1,3-dioxolan-2-yl)phenyl]carbamoyl}-2-methylpyrazole-3-carboxylate (500.0 mg, 1.509 mmol, 1 equiv) in H₂O (3 mL) and THF (3 mL) was added LiOH (90.4 mg, 3.772 mmol, 2.5 equiv) at room temperature. The resulting mixture was stirred for 2 h. The mixture was acidified to pH 6 with HCl (1 mol/L). The aqueous layer was extracted with EA (3×50 mL). The resulting mixture was concentrated under reduced pressure to afford 5-{[2-(1,3-dioxolan-2-yl)phenyl]carbamoyl}-2-methylpyrazole-3-carboxylic acid (170.0 mg, 35.50%) as a light yellow oil. MS (ESI) calcd. for C₁₅H₁₅N₃O₅, 317.10 m/z, found 318.10 [M+H]⁺.

### Step 4: Synthesis of (1R,3S)-3-[5-(5-{[2-(1,3-dioxolan-2-yl)phenyl]carbamoyl}-2-methyl pyrazole-3-amido)-2H-pyrazol-3-yl]cyclopentyl N-isopropylcarbamate

To a stirred solution of 5-{[2-(1,3-dioxolan-2-yl)phenyl]carbamoyl}-2-methylpyrazole-3-carboxylic acid (151.0 mg, 0.476 mmol, 1.2 equiv) and (1R,3S)-3-(5-amino-2H-pyrazol-3-yl)cyclopentyl N-isopropylcarbamate (100.1 mg, 0.397 mmol, 1.0 equiv) in pyridine (5 mL) was added HBTU (180.5 mg, 0.476 mmol, 1.2 equiv) at room temperature. The resulting mixture was stirred for 10 h at 110° C., then cooled to room temperature and diluted with water. The resulting mixture was extracted with ethyl acetate (3×50 mL). The organic layers were combined, dried over anhydrous sodium sulfate, filtered, and concentrated under reduced pressure. The residue obtained was purified by silica gel chromatography (65% ethyl acetate/petroleum ether) to afford (1R,3S)-3-[5-(5-{[2-(1,3-dioxolan-2-yl)phenyl]carbamoyl}-2-methylpyrazole-3-amido)-2H-pyrazol-3-yl]cyclopentyl N-isopropyl-carbamate (120.0 mg, 54.86%) as a light yellow oil. MS (ESI) calcd. for C₂₇H₃₃N₇O₆, 551.25 m/z, found 552.20 [M+H]⁺.

### Step 5: Synthesis of (1R,3S)-3-(5-{5-[(2-formylphenyl)carbamoyl]-2-methylpyrazole-3-amido}-2H-pyrazol-3-yl)cyclopentyl N-isopropylcarbamate trifluoroacetic acid

To a stirred solution of (1R,3S)-3-[5-(5-{[2-(1,3-dioxolan-2-yl)phenyl]carbamoyl}-2-methylpyrazole-3-amido)-2H-pyrazol-3-yl]cyclopentyl N-isopropylcarbamate (50.0 mg, 0.091 mmol, 1 equiv) in DCM (2 mL, 31.461 mmol, 347.08 equiv) were added TFA (0.5 mL) at room temperature. The resulting mixture was stirred for 2 h at this temperature. The resulting mixture was concentrated under reduced pressure. The crude product was purified by Prep-HPLC with the following conditions (Column: XBridge Shield RP 18 OBD Column, 30×150 mm, 5 μm; Mobile Phase A: Water (0.05% TFA), Mobile Phase B: ACN; Flow rate: 60 mL/min; Gradient: 32% B to 62% B in 7 min; Wave Length: 254 nm; RT1 (min): 5.63) to afford (1R,3S)-3-(5-{5-[(2-formylphenyl)carbamoyl]-2-methylpyrazole-3-amido}-2H-pyrazol-3-yl)cyclopentyl N-isopropylcarbamate; trifluoroacetic acid (4.7 mg, 7.68%) as a light yellow solid. MS (ESI) calcd. for C₂₅H₂₉N₇O₅, 507.22 m/z, found 508.25 [M+H]⁺. ¹H NMR (400 MHz, DMSO-d₆) δ 12.29 (br s, 1H), 11.54 (s, 1H), 10.84 (s, 1H), 10.37 (s, 1H), 7.52-7.56 (m, 1H), 6.96-6.98 (m, 1H), 6.88 (s, 1H), 6.75-6.77 (m, 1H), 6.59-6.61 (m, 1H), 6.42 (s, 1H), 4.98-5.03 (m, 1H), 4.02 (s, 3H), 3.51-3.60 (m, 1H), 3.05-3.09 (m, 1H), 2.46-2.50 (m, 1H), 1.90-2.04 (m, 2H), 1.47-1.78 (m, 3H), 1.04 (d, J=6.4 Hz, 6H).

### Example 13: Synthesis of Compound 28: (1R,3S)-3-(3-(3-((2-formyl-3-hydroxybenzyl)oxy)-1-methyl-1H-pyrazole-5-carboxamido)-1H-pyrazol-5-yl)cyclopentyl isopropylcarbamate

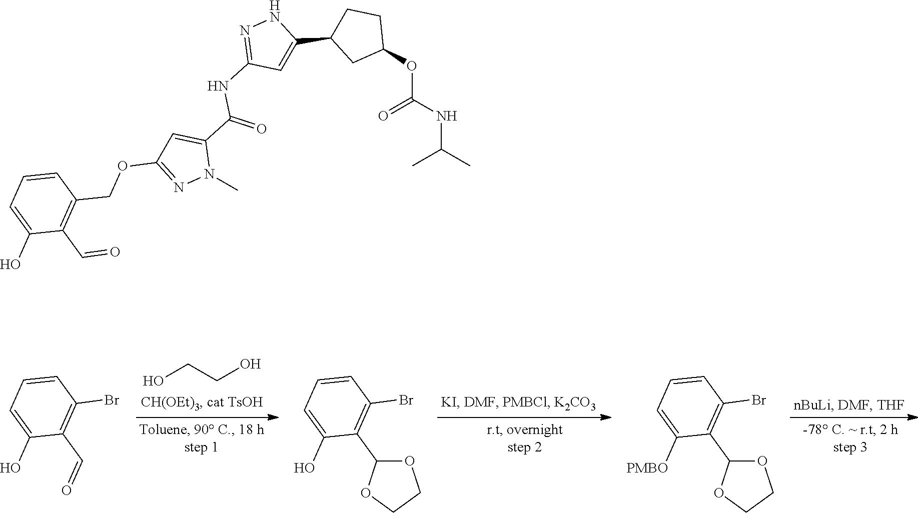

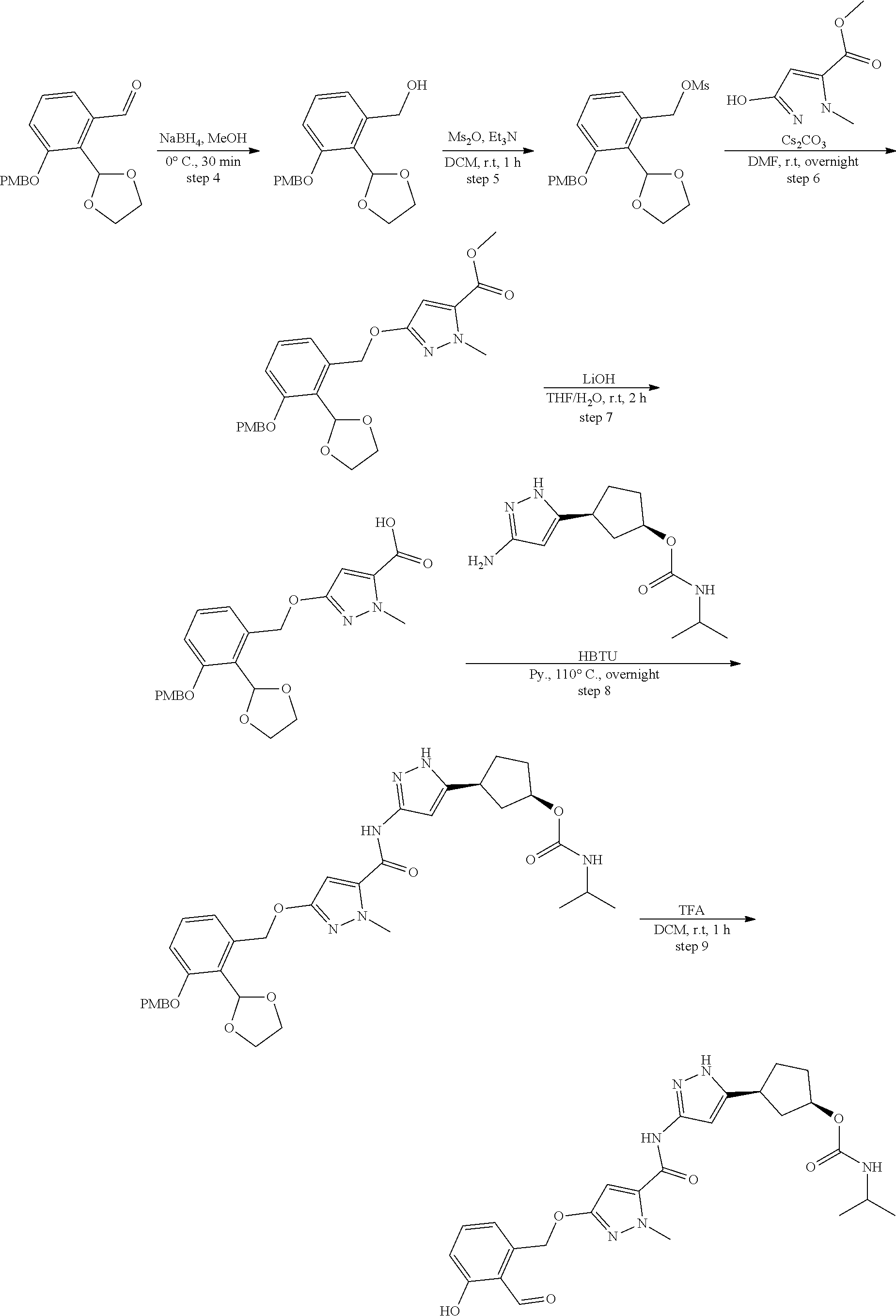

### Step 1: Synthesis of 3-bromo-2-(1,3-dioxolan-2-yl)phenol

To a stirred solution of 2-bromo-6-hydroxybenzaldehyde (25.0 g, 124.366 mmol, 1 equiv) and TsOH (2.1 g, 12.437 mmol, 0.1 equiv) in toluene (100 mL) were added tri ethyl orthoformate (110.5 g, 746.196 mmol, 6.0 equiv), ethylene glycol (92.6 g, 1492.392 mmol, 12 equiv) and stirred for 18 h at 90° C. under nitrogen atmosphere. Desired product could be detected by LCMS. The reaction mixture was then quenched with H₂O (200 mL) and extracted with EA (200 mL×3). The combined extracts washed with brine (50 mL), dried over anhydrous Na₂SO₄, filtered, and concentrated to dryness under vacuum. The residue was purified by silica gel column chromatography, eluted with EA/PE (0-30%) to afford 3-bromo-2-(1,3-dioxolan-2-yl)phenol (12.6 g 80.04%) as a red solid. MS (ESI) calcd. For C₉H₉BrO₃, 243.97 m/z, found 245.03 [M+H]⁺.

### Step 2: Synthesis of 2-(2-bromo-6-((4-methoxybenzyl)oxy)phenyl)-1,3-dioxolane

To a stirred solution of 3-bromo-2-(1,3-dioxolan-2-yl)phenol (3.0 g, 12.241 mmol, 1 equiv), KI (230 mg, 1.224 mmol, 0.1 equiv) and K₂CO₃ (1.7 g, 12.241 mmol, 1 equiv) in DMF (30 mL) was added PMBCl (3.8 g, 24.482 mmol, 2 equiv) and stirred overnight at r.t under nitrogen atmosphere. Desired product could be detected by LCMS. The reaction mixture was then quenched with H₂O (60 mL) and extracted with EA (30 mL×3). The combined extracts washed with brine (30 mL), dried over anhydrous Na₂SO₄, filtered, and concentrated to dryness under vacuum. The residue was purified by silica gel column chromatography, eluted with EA/PE (0-50%) to afford 2-{2-bromo-6-[(4-methoxyphenyl)methoxy]phenyl}-1,3-dioxolane (3.0 g, 62.40%) as a yellow solid. MS (ESI) calcd. For C₁₇H₁₇BrO₄, 364.03 m/z, found 365.03[M+H]⁺.

### Step 3: Synthesis of 2-(1,3-dioxolan-2-yl)-3-((4-methoxybenzyl)oxy)benzaldehyde

A solution of 2-{2-bromo-6-[(4-methoxyphenyl)methoxy]phenyl}-1,3-dioxolane (3.0 g, 8.214 mmol, 1 equiv) in THF (50 mL) was treated with n-butyllithium (4.2 mL, 10.678 mmol, 1.30 equiv, 2.5 M in hexane) and stirred for 15 mins at −78° C. under nitrogen atmosphere. To the above mixture DMF (20 mL) was added and stirred for 2 h at r.t. Desired product could be detected by LCMS. The reaction mixture was then quenched with NH₄Cl (100 mL) and extracted with EA (50 mL×3), and the combined extracts washed with brine (50 mL), dried over anhydrous Na₂SO₄, filtered, and concentrated to dryness under vacuum. The residue was purified by silica gel column chromatography, eluted with EA/PE (0-40%) to afford 2-(1,3-dioxolan-2-yl)-3-[(4-methoxyphenyl)methoxy]benzaldehyde (2.0 g, 77.46%) as a white solid. MS (ESI) calcd. For C₁₈H₁₈O₅, 314.11 m/z, found 315.11 [M+H]⁺.

### Step 4: Synthesis of (2-(1,3-dioxolan-2-yl)-3-((4-methoxybenzyl)oxy)phenyl)methanol

NaBH₄ (60.2 mg, 1.591 mmol, 0.5 equiv) was added to the solution of 2-(1,3-dioxolan-2-yl)-3-[(4-methoxyphenyl)methoxy]benzaldehyde (1.0 g, 3.181 mmol, 1 equiv) in MeOH (20 mL) and stirred for 30 mins at 0° C. Desired product could be detected by LCMS. The reaction mixture was then treated with NaHCO₃ (40 mL) and extracted with DCM (30 mL×3), and the combined extracts washed with brine (20 mL), dried over anhydrous Na₂SO₄, filtered, and concentrated to dryness under vacuum. The residue was purified by silica gel column chromatography, eluted with EA/PE (0-50%) to afford [2-(1,3-dioxolan-2-yl)-3-[(4-methoxyphenyl)methoxy]phenyl]methanol (940.0 mg, 93.40%) as a white solid. MS (ESI) calcd. For C₁₈H₂₀O₅, 316.13 m/z, found 317.10 [M+H]⁺.

### Step 5: Synthesis of 2-(1,3-dioxolan-2-yl)-3-((4-methoxybenzyl)oxy)benzyl methanesulfonate

Methanesulfonic anhydride (619.8 mg, 3.558 mmol, 1.2 equiv) was added to the solution of [2-(1,3-dioxolan-2-yl)-3-[(4-methoxyphenyl)methoxy]phenyl]methanol (938.0 mg, 2.965 mmol, 1 equiv) and triethylamine (450.1 mg, 4.447 mmol, 1.5 equiv) in DCM (20 mL) at 0° C. and stirred at r.t for 1 h under nitrogen atmosphere. Desired product could be detected by LCMS. The reaction mixture was then quenched with H₂O (40 mL) and extracted with DCM (30 mL×3), and the combined extracts washed with brine (20 mL), dried over anhydrous Na₂SO₄, filtered, and concentrated to dryness under vacuum. The residue was purified by silica gel column chromatography, eluted with EA/PE (0-60%) to afford [2-(1,3-dioxolan-2-yl)-3-[(4-methoxyphenyl)methoxy]phenyl]methyl methanesulfonate (600.0 mg, 51.30%) as a white solid. MS (ESI) calcd. For C₁₉H₂₂O₇S, 394.10 m/z, found 395.10 [M+H]⁺.

### Step 6: Synthesis of methyl 3-((2-(1,3-dioxolan-2-yl)-3-((4-methoxybenzyl)oxy)benzyl)oxy)-1-methyl-1H-pyrazole-5-carboxylate

A solution of methyl 5-hydroxy-2-methylpyrazole-3-carboxylate (250.0 mg, 1.601 mmol, 1.00 equiv), Cs₂CO₃ (1.3 g, 4.002 mmol, 2.5 equiv) dissolved in DMF (5 mL) and stirred for 20 mins at r.t. Following this, [2-(1,3-dioxolan-2-yl)-3-[(4-methoxyphenyl)methoxy]phenyl]methyl methanesulfonate (821.0 mg, 2.081 mmol, 1.3 equiv) was added in portions at r.t. The resulting mixture was stirred overnight at r.t. Desired product could be detected by LCMS. The reaction mixture was then quenched with H₂O (20 mL) and extracted with EA (30 mL×3), and the combined extracts washed with brine (10 mL), dried over anhydrous Na₂SO₄, filtered, and concentrated to dryness under vacuum. The residue was purified by silica gel column chromatography, eluted with EA/PE (0-30%) to afford methyl 5-{[2-(1,3-dioxolan-2-yl)-3-[(4-methoxyphenyl)methoxy]phenyl]methoxy}-2-methylpyrazole-3-carboxylate (600.0 mg, 82.45%) as a white solid. MS (ESI) calcd. For C₂₄H₂₆N₂O₄, 454.17 m/z, found 455.15 [M+H]⁺.

### Step 7: Synthesis of 3-((2-(1,3-dioxolan-2-yl)-3-((4-methoxybenzyl)oxy)benzyl)oxy)-1-methyl-1H-pyrazole-5-carboxylic acid

Methyl 5-{[2-(1,3-dioxolan-2-yl)-3-[(4-methoxyphenyl)methoxy]phenyl]methoxy}-2-methyl pyrazole-3-carboxylate (600.0 mg, 1.320 mmol, 1 equiv), LiOH (79.1 mg, 3.300 mmol, 2.5 equiv) were dissolved in THF (10 mL), H₂O (3 mL) and stirred for 2 h at r.t. Desired product could be detected by LCMS. The mixture was acidified to pH 6 with HCl (2 mol/L) and mixture was extracted with EA (10 mL×3), and the combined extracts washed with brine (10 mL), dried over anhydrous Na₂SO₄, filtered, and concentrated to dryness under vacuum. The resulting mixture was concentrated under reduced pressure to afford 5-{[2-(1,3-dioxolan-2-yl)-3-[(4-methoxyphenyl)methoxy]phenyl]methoxy}-2-methylpyrazole-3-carboxylic acid (400.0 mg, 68.79%) as a white solid. MS (ESI) calcd. For C₂₃H₂₄N₂O₇, 440.15 m/z, found 441.155 [M+H]⁺.

### Step 8: Synthesis of (1R,3S)-3-(3-(3-((2-(1,3-dioxolan-2-yl)-3-((4-methoxybenzyl)oxy) benzyl)oxy)-1-methyl-1H-pyrazole-5-carboxamido)-1H-pyrazol-5-yl)cyclopentyl isopropylcarbamate

A solution of 5-{[2-(1,3-dioxolan-2-yl)-3-[(4-methoxyphenyl)methoxy]phenyl]methoxy}-2-methylpyrazole-3-carboxylic acid (209.5 mg, 0.476 mmol, 1.20 equiv) in pyridine (10 mL) was treated with (1R,3S)-3-(5-amino-2H-pyrazol-3-yl)cyclopentyl N-isopropylcarbamate (100.0 mg, 0.396 mmol, 1.00 equiv) and HBTU (150.3 mg, 0.594 mmol, 1.50 equiv) and stirred overnight at 110° C. Desired product could be detected by LCMS. The reaction mixture was then quenched with H₂O (20 mL) and extracted with EA (20 mL×3), and the combined extracts washed with brine (10 mL), dried over anhydrous Na₂SO₄, filtered, and concentrated to dryness under vacuum. The residue was purified by silica gel column chromatography, eluted with MeOH/DCM (0-20%) to afford (1R,3S)-3-[5-(5-{[2-(1,3-dioxolan-2-yl)-3-[(4-methoxyphenyl)methoxy]phenyl]methoxy}-2-methylpyrazole-3-amido)-2H-pyrazol-3-yl]cyclopentyl N-isopropylcarbamate (100.0 mg, 37.39%) as a yellow solid. MS (ESI) calcd. For C₃₅H₄₂N₆O₈, 674.30 m/z, found 675.15 [M+H]⁺.

### Step 9: Synthesis of (1R,3S)-3-(3-(3-((2-formyl-3-hydroxybenzyl)oxy)-1-methyl-1H-pyrazole-5-carboxamido)-1H-pyrazol-5-yl)cyclopentyl isopropylcarbamate

(1R,3S)-3-[5-(5-{[2-(1,3-dioxolan-2-yl)-3-[(4-methoxyphenyl)methoxy]phenyl]methoxy}-2-methylpyrazole-3-amido)-2H-pyrazol-3-yl]cyclopentyl N-isopropylcarbamate (100.0 mg, 0.148 mmol, 1 equiv) in DCM (6 mL) and TFA (2 mL) was stirred for 1 h at r.t. Desired product could be detected by LCMS. The resulting mixture was concentrated under reduced pressure and dissolved in DMF (1 mL). The crude product was then purified by Prep-HPLC with the following conditions: Column: Xselect CSH OBD Column, 30×150 mm, 5 μm; Mobile Phase A: Water (0.1% FA), Mobile Phase B: ACN; Flow rate: 60 mL/min; Gradient: 36% B to 59% B in 10 min; Wave Length: 254/220 nm. This resulted in (1R,3S)-3-(5-{5-[(2-formyl-3-hydroxyphenyl) methoxy]-2-methylpyrazole-3-amido}-2H-pyrazol-3-yl)cyclopentyl N-isopropyl carbamate (1.3 mg, 1.58%) as a white solid. MS (ESI) calcd. For C₂₅H₃₀N₆O₆, 510.20 m/z, found 511.15 [M+H]⁺. ¹H NMR (300 MHz, DMSO-d₆) δ (ppm): 12.25 (s, 1H), 11.08 (s, 1H), 10.70 (s, 1H), 10.47 (s, 1H), 7.57-7.49 (m, 1H), 7.09 (d, J=7.6 Hz, 1H), 7.02-6.96 (m, 2H), 6.65 (s, 1H), 6.41 (s, 1H), 5.47 (s, 2H), 5.00 (s, 1H), 3.95 (s, 3H), 3.63-3.49 (m, 1H), 3.12-3.03 (m, 1H), 2.32 (s, 1H), 2.02 (s, 1H), 1.73 (d, J=7.9 Hz, 3H), 1.61 (s, 1H), 1.04 (d, J=6.6 Hz, 6H).

### Example 14: Synthesis of Compound 24: (1R,3S)-3-(3-((4-((2-formyl-3-hydroxyphenoxy)methyl) pyridin-2-yl)amino)-1H-pyrazol-5-yl)cyclopentyl isopropylcarbamate

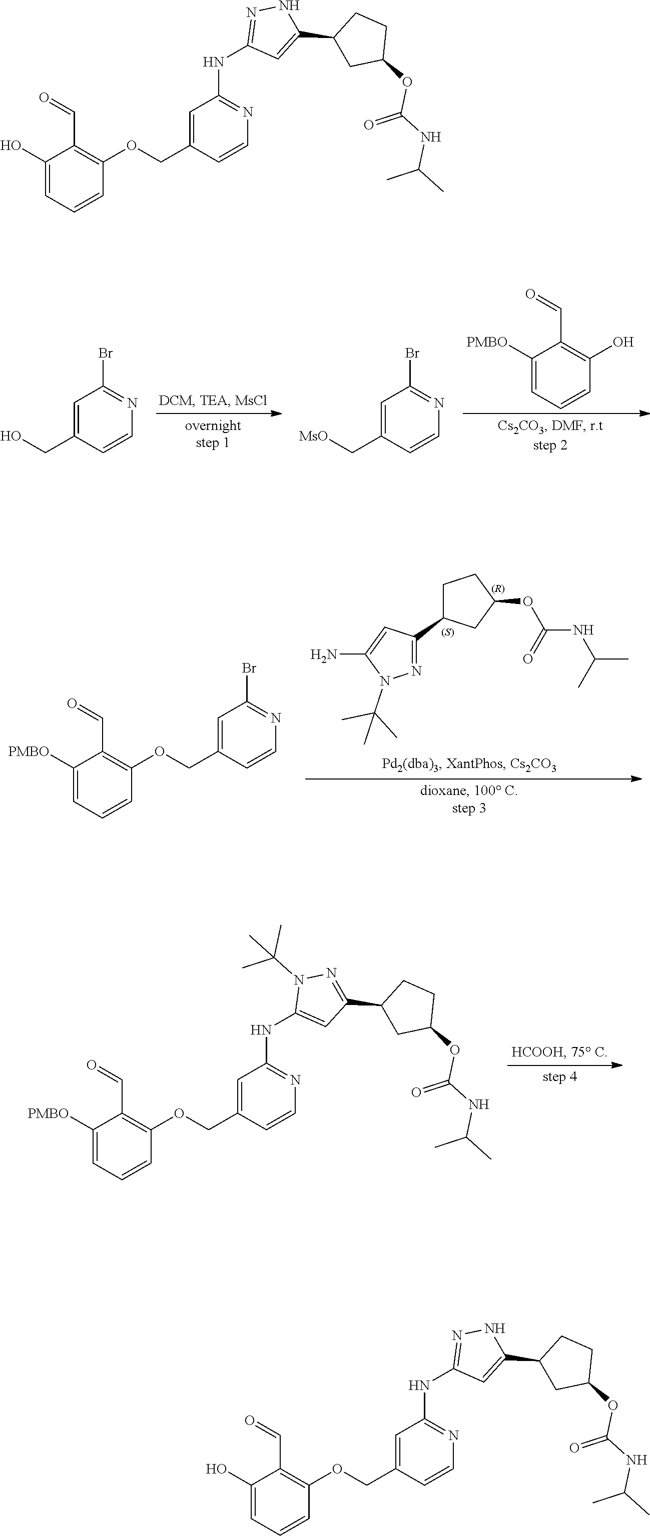

### Step 1: Synthesis of (2-bromopyridin-4-yl)methyl methanesulfonate

To a stirred solution of (2-bromopyridin-4-yl)methanol (4 g, 21.274 mmol, 1 equiv) in DCM (40 mL) were added triethylamine (6.46 g, 63.822 mmol, 3 equiv) at 0° C. The resulting mixture was stirred for 10 min at 0° C., then MsCl (7.31 g, 63.822 mmol, 3 equiv) was added and the mixture was allowed to warm to room temperature and stirred for 1 h. The reaction mixture was quenched with water and extracted with EA (3×100 mL). The combined organic layers were washed with sat. NaHCO₃ (2×100 mL), dried over anhydrous Na₂SO₄ and concentrated to dryness in vacuo to give (2-bromopyridin-4-yl)methyl methanesulfonate (2.8 g, 49.46%) as a brown oil. MS (ESI) calcd. for C₇H₈BrNO₃S, 264.94 m/z, found 265.90 [M+H]⁺.

### Step 2: Synthesis of 2-((2-bromopyridin-4-yl)methoxy)-6-((4-methoxybenzyl)oxy) benzaldehyde

To a stirred solution of 2-hydroxy-6-[(4-methoxyphenyl)methoxy]benzaldehyde (0.49 g, 1.879 mmol, 1 equiv) and Cs₂CO₃ (1.53 g, 4.697 mmol, 2.5 equiv) in DMF (5 mL) were stirred for 0.5 h at room temperature. Then to the mixture was added (2-bromopyridin-4-yl)methyl methanesulfonate (1.5 g, 5.637 mmol, 3 equiv) and stirred for 2 h at room temperature. The resulting mixture was diluted with water and extracted with EA (3×100 mL). The combined organic layers were washed with brine (2×100 mL), dried over anhydrous Na₂SO₄. After filtration, the filtrate was concentrated under reduced pressure. The crude product was purified by silica gel column chromatography, eluted with EA:PE (1:1) to afford 2-[(2-bromopyridin-4-yl)methoxy]-6-[(4-methoxyphenyl)methoxy]benzaldehyde (0.35 g, 43.49%) as a brown oil. MS (ESI) calcd. for C₂₁H₁₈BrNO₄, 427.04 m/z, found 428.00 [M+H]⁺.

### Step 3: Synthesis of (1R,3S)-3-(1-(tert-butyl)-5-((4-((2-formyl-3-((4-methoxybenzyl)oxy) phenoxy)methyl)pyridin-2-yl)amino)-1H-pyrazol-3-yl)cyclopentyl isopropylcarbamate

To a solution of 2-[(2-bromopyridin-4-yl)methoxy]-6-[(4-methoxyphenyl)methoxy]benzaldehyde (200 mg, 0.467 mmol, 1 equiv) and (1R,3S)-3-(5-amino-1-tert-butylpyrazol-3-yl)cyclopentyl N-isopropylcarbamate (144.0 mg, 0.467 mmol, 1 equiv) in dioxane (8 mL) was added Pd₂(dba)₃ (64.1 mg, 0.070 mmol, 0.15 equiv), XantPhos (81.1 mg, 0.140 mmol, 0.3 equiv), Cs₂CO₃ (380.4 mg, 1.167 mmol, 2.5 equiv) sequentially. The resulting mixture was maintained under nitrogen and stirred for 3 h at 80° C. The reaction progress was monitored by LCMS. After cooling down to room temperature, the reaction was quenched with water. The resulting mixture was extracted with ethyl acetate. The organic layers were combined, dried over anhydrous sodium sulfate, filtered and concentrated. The residue obtained was purified by silica gel chromatography (0-90% EA/PE) to afford (1R,3S)-3-{1-tert-butyl-5-[(4-{2-formyl-3-[(4-methoxyphenyl)methoxy]phenoxymethyl}pyridin-2-yl)amino]pyrazol-3-yl}cyclopentyl N-isopropylcarbamate (60.0 mg, 19.59%). MS (ESI) calcd. for C₃₇H₄₅N₅O₆, 655.34 m/z, found 656.45 [M+H]⁺.

### Step 4: Synthesis of (1R,3S)-3-(3-((4-((2-formyl-3-hydroxyphenoxy)methyl)pyridin-2-yl)amino)-1H-pyrazol-5-yl)cyclopentyl isopropylcarbamate

(1R,3S)-3-{1-tert-butyl-5-[(4-{2-formyl-3-[(4-methoxyphenyl)methoxy]phenoxymethyl}pyridine-2-yl)amino]pyrazol-3-yl}cyclopentyl N-isopropylcarbamate (60.0 mg, 0.091 mmol, 1 equiv) in HCOOH (2 mL) was stirred for 1 h at 75° C. The mixture was concentrated under reduced pressure. The crude product was purified by HPLC (Column: Xselect CSH OBD Column, 30×150 mm, 5 um; Mobile Phase A: Water (0.1% FA), Mobile Phase B: ACN; Flow rate: 60 mL/min; Gradient: 15% B to 39% B in 10 min; Wave Length: 254 nm/220 nm; RT1 (min): 8.5) to afford (1R,3S)-3-(5-{[4-(2-formyl-3-hydroxyphenoxymethyl)pyridin-2-yl]amino}-2H-pyrazol-3-yl)cyclopentyl N-isopropylcarbamate (2.8 mg, 6.34%) as a white solid. MS (ESI) calcd. for C₂₅H₂₉N₅O₅, 479.22 m/z, found 480.20 [M+H]⁺. ¹H NMR (400 MHz, DMSO-d₆) δ 12.17 (br s, 1H), 11.78 (s, 1H), 10.45 (s, 1H), 9.50 (br s, 1H), 8.10-8.15 (m, 1H), 7.53 (t, J=8.0 Hz, 1H), 7.30 (s, 1H), 6.95 (d, J=7.2 Hz, 1H), 6.81 (d, J=4.8 Hz, 1H), 6.66 (d, J=8.4 Hz, 1H), 6.56 (d, J=8.4 Hz, 1H), 6.13 (s, 1H), 5.24 (s, 2H), 4.92-5.11 (m, 1H), 3.51-3.68 (m, 1H), 2.98-3.16 (m, 1H), 2.40-2.48 (m, 1H), 1.97-2.10 (m, 1H), 1.84-1.96 (m, 1H), 1.55-1.81 (m, 3H), 1.03 (d, J=6.4 Hz, 6H).

### Example 15: Synthesis of Compound 19: (1R,3S)-3-{5-[5-(2-formyl-3-hydroxyphenoxy)-2-methylpyrazole-3-amido]-2H-pyrazol-3-yl}cyclopentyl N-isopropylcarbamate

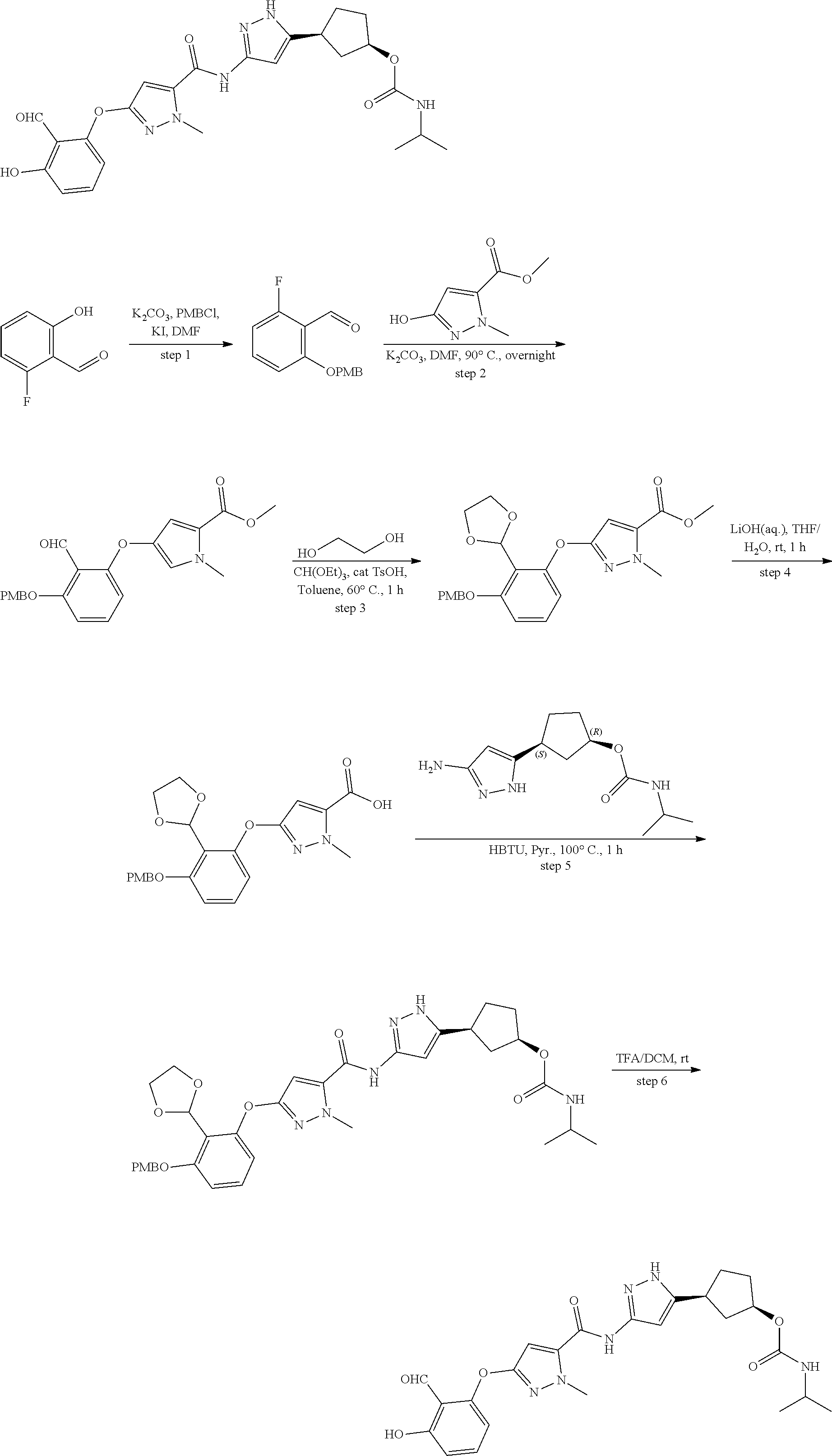

### Step 1: Synthesis of 2-fluoro-6-((4-methoxybenzyl)oxy)benzaldehyde

To a stirred solution of 2-fluoro-6-hydroxybenzaldehyde (15 g, 107.056 mmol, 1 equiv) in N,N-dimethylformamide (DMF) (130 mL) was added K₂CO₃ (36.99 g, 267.640 mmol, 2.5 equiv) at 22° C. The resulting mixture was stirred for 20 min at 0° C. To the above mixture was added PMBCl (21.80 g, 139.173 mmol, 1.3 equiv) dropwise at 0° C. The resulting mixture was stirred for additional 1 h at 60° C., then cooled to room temperature and diluted with water. The resulting mixture was extracted with EA (3×200 mL). The combined organic layers were washed with brine (3×200 mL), dried over anhydrous Na₂SO₄. The resulting mixture was concentrated under vacuum. The residue was purified by silica gel column chromatography, eluted with PE/EA (1:1) to afford 2-fluoro-6-[(4-methoxyphenyl)methoxy]benzaldehyde (24.8 g, 86.16%) as an off-white solid. MS (ESI) calcd. for C₁₅H₁₃FO₃, 260.08 m/z, found 261.35 [M+H]⁺

### Step 2: Synthesis of methyl 5-{2-formyl-3-[(4-methoxyphenyl)methoxy]phenoxy}-2-methyl pyrazole-3-carboxylate

To a stirred solution of 2-fluoro-6-[(4-methoxyphenyl)methoxy]benzaldehyde (1.5 g, 5.763 mmol, 1 equiv) and methyl 5-hydroxy-2-methylpyrazole-3-carboxylate (0.9 g, 5.763 mmol, 1 equiv) in DMF (20 mL) was added K₂CO₃ (1.6 g, 11.526 mmol, 2 equiv) at room temperature. The resulting mixture was stirred for 1 h at 90° C., then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by silica gel column chromatography, eluted with EA:PE (2:3) to afford methyl 5-{2-formyl-3-[(4-methoxyphenyl) methoy]phenoxy}-2-methylpyrazole-3-carboxylate (1.2 g, 52.53%) a yellow oil. MS (ESI) calcd. for C₂₁H₂₀N₂O₆, 396.13 m/z, found 397.15 [M+H]⁺.

### Step 3: Synthesis of methyl 5-[2-(1,3-dioxolan-2-yl)-3-[(4-methoxyphenyl)methoxy]phenoxy]-2-methylpyrazole-3-carboxylate)

To a stirred solution of methyl 5-{2-formyl-3-[(4-methoxyphenyl)methoxy]phenoxy}-2-methyl pyrazole-3-carboxylate (900.0 mg, 2.270 mmol, 1 equiv) and ethylene glycol (0.63 mL, 11.350 mmol, 5 equiv) in toluene (30 mL) was added Triethyl orthoformate (1.13 mL, 6.810 mmol, 3 equiv) and TsOH (39.1 mg, 0.227 mmol, 0.1 equiv) at room temperature. The resulting mixture was stirred for 2 h at 60° C., then cooled to room temperature and diluted with water. The resulting mixture was extracted with EA (3×100 mL). The combined organic layers were washed with brine (3×100 mL), dried over anhydrous Na₂SO₄. After filtration, the filtrate was concentrated under reduced pressure. The mixture was purified by silica gel column chromatography, eluted with EA/PE (2:3) to afford methyl 5-[2-(1,3-dioxolan-2-yl)-3-[(4-methoxyphenyl)methoxy]phenoxy]-2-methylpyrazole-3-carboxylate) (500.0 mg, 50.00%) as a yellow solid. MS (ESI) calcd. for C₂₃H₂₄N₂O₇, 440.16 m/z, found 440.95 [M+H]⁺.

### Step 4: Synthesis of 5-[2-(1,3-dioxolan-2-yl)-3-[(4-methoxyphenyl)methoxy]phenoxy]-2-methylpyrazole-3-carboxylic acid

To a stirred solution of methyl 5-[2-(1,3-dioxolan-2-yl)-3-[(4-methoxyphenyl)methoxy]phenoxy]-2-methyl pyrazole-3-carboxylate (480.0 mg, 1.090 mmol, 1 equiv) in THF (2 mL) and H₂O (2 mL) was added LiOH (65.3 mg, 2.725 mmol, 2.5 equiv) at room temperature. The resulting mixture was stirred for 1 h. Desired product 5-[2-(1,3-dioxolan-2-yl)-3-[(4-methoxyphenyl)methoxy]phenoxy]-2-methylpyrazole-3-carboxylic acid was detected by LCMS. The mixture was acidified to pH 6 with HCl (1 moL/L). The aqueous layer was extracted with EA (3×50 mL). The resulting mixture was concentrated under reduced pressure to afford 5-[2-(1,3-dioxolan-2-yl)-3-[(4-methoxyphenyl)methoxy]phenoxy]-2-methylpyrazole-3-carboxylic acid (300.0 mg, 64.56%) as a yellow solid. MS (ESI) calcd. for C₂₂H₂₂N₂O₇, 426.14 m/z, found 427.15[M+H]⁺.

### Step 5: Synthesis of (1R,3S)-3-(5-{5-[2-(1,3-dioxolan-2-yl)-3-[(4-methoxyphenyl)methoxy]phenoxy]-2-methyl pyrazole-3-amido}-2H-pyrazol-3-yl)cyclopentyl N-isopropylcarbamate

To a stirred solution of 5-[2-(1,3-dioxolan-2-yl)-3-[(4-methoxyphenyl)methoxy]phenoxy]-2-methylpyrazole-3-carboxylic acid (200.0 mg, 0.469 mmol, 1 equiv) and (1R,3S)-3-(5-amino-2H-pyrazol-3-yl)cyclopentyl N-isopropylcarbamate (118.3 mg, 0.469 mmol, 1 equiv) in pyridine (3 mL) was added HBTU (213.4 mg, 0.563 mmol, 1.2 equiv) at room temperature. The resulting mixture was stirred for 2 h at 100° C. Desired product (1R,3S)-3-(5-{5-[2-(1,3-dioxolan-2-yl)-3-[(4-methoxyphenyl)methoxy]phenoxy]-2-methylpyrazole-3-amido}-2H-pyrazol-3-yl)cyclo-pentyl N-isopropylcarbamate was detected by LCMS. The mixture was acidified to pH 6 with HCl (1 mol/L). The aqueous layer was extracted with EA (3×50 mL). The resulting mixture was concentrated under reduced pressure to afford (1R,3S)-3-(5-{5-[2-(1,3-dioxolan-2-yl)-3-[(4-methoxy phenyl)methoxy]phenoxy]-2-methylpyrazole-3-amido}-2H-pyrazol-3-yl)cyclopentyl N-isopropylcarbamate (90.0 mg, 29.04%) as a yellow solid. MS (ESI) calcd. for C₃₄H₄₀N₆O₈, 660.29 m/z, found 661.30 [M+H]⁺.

### Step 6: Synthesis of (1R,3S)-3-{5-[5-(2-formyl-3-hydroxyphenoxy)-2-methylpyrazole-3-amido]-2H-pyrazol-3-yl}cyclopentyl N-isopropylcarbamate

To a stirred solution of (1R,3S)-3-(5-{5-[2-(1,3-dioxolan-2-yl)-3-[(4-methoxyphenyl)methoxy]phenoxy]-2-methylpyrazole-3-amido}-2H-pyrazol-3-yl)cyclopentyl N-isopropylcarbamate (71.0 mg, 0.107 mmol, 1 equiv) in DCM (2 mL) were added TFA (0.5 mL) at room temperature. The resulting mixture was stirred for 0.5 h at this temperature. The resulting mixture was concentrated under reduced pressure. The crude product was purified by Prep-HPLC with the following conditions (Column: XBridge Prep Shield RP18 OBD Column, 30×150 mm, 5 μm; Mobile Phase A: Water (0.05% TFA), Mobile Phase B: ACN; Flow rate: 60 mL/min; Gradient: 32% B to 62% B in 7 min; Wave Length: 254 nm/220 nm; RT1 (min): 5.98) to afford (1R,3S)-3-{5-[5-(2-formyl-3-hydroxyphenoxy)-2-methylpyrazole-3-amido]-2H-pyrazol-3-yl}cyclopentyl N-isopropyl-carbamate (2.7 mg, 4.97%) as a light yellow solid. MS (ESI) calcd. for C₂₄H₂₈N₆O₆, 496.21 m/z, found 497.30[M+H]⁺. ¹H NMR (400 MHz, DMSO-d₆) δ 12.29 (br s, 1H), 11.54 (s, 1H), 10.84 (s, 1H), 10.37 (s, 1H), 7.52-7.56 (m, 1H), 6.96-6.98 (m, 1H), 6.88 (s, 1H), 6.75-6.77 (m, 1H), 6.59-6.61 (m, 1H), 6.42 (s, 1H), 4.98-5.03 (m, 1H), 4.02 (s, 3H), 3.51-3.60 (m, 1H), 3.05-3.09 (m, 1H), 2.46-2.50 (m, 1H), 1.90-2.04 (m, 2H), 1.47-1.78 (m, 3H), 1.04 (d, J=6.4 Hz, 6H).

### Example 16: Synthesis of Compound 91: (1R,3S)-3-(3-((2-(2-formyl-3-hydroxyphenyl)pyrazolo[1,5-a]pyridin-4-yl)amino)-1H-pyrazol-5-yl)cyclopentyl isopropylcarbamate

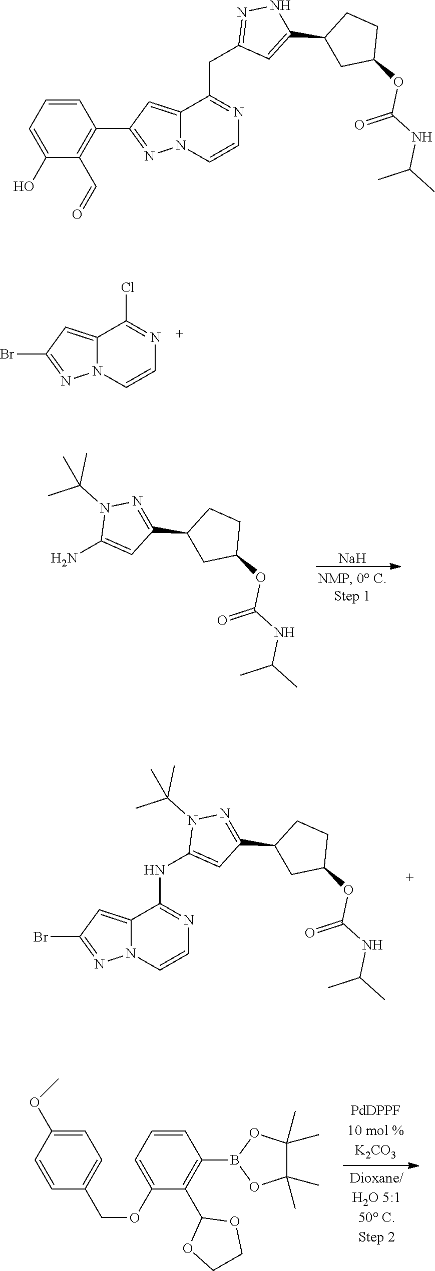

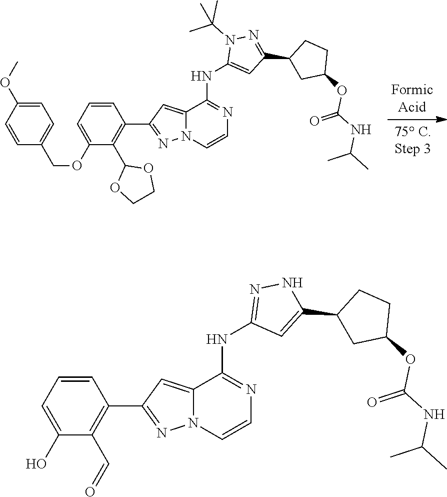 [SMILES: COc1ccc(COc2cccc(-c3cc4c(Nc5cc([C@H]6CC[C@@H](OC(=O)NC(C)C)C6)nn5C(C)(C)C)nccn4n3)c2C2OCCO2)cc1 ; CC(C)NC(=O)O[C@@H]1CC[C@H](c2cc(Nc3nccn4nc(-c5cccc(O)c5C=O)cc34)n[nH]2)C1]

### Step 1: Synthesis of (1R,3S)-3-(5-((2-bromopyrazolo[1,5-a]pyrazin-4-yl)amino)-1-(tert-butyl)-1H-pyrazol-3-yl)cyclopentyl isopropylcarbamate

A 40 mL reaction vial was charged with 2-bromo-4-chloropyrazolo[1,5-a]pyrazine (260 mg, 1.12 mmol, 1 equiv), (1R,3S)-3-(5-amino-1-(tert-butyl)-1H-pyrazol-3-yl)cyclopentyl isopropyl-carbamate (345 mg, 1.12 mmol, 1 equiv), 2.5 mL N-methylpyrrolidone and cooled to 0° C. with an ice water bath. Sodium hydride, 60% dispersion in oil (134 mg, 3.36 mmol, 3 equiv) was then added in portions and the reaction allowed to stir for 3 h at 0° C. The reaction mixture was then quenched with 5 mL of a saturated solution of ammonium chloride and further diluted with 5 mL water. The reaction mixture was extracted with EtOAc (3×10 mL), dried over sodium sulphate, filtered, and concentrated to dryness. The crude material was purified by silica gel column chromatography, 0 to 100% EtOAc/Hex, desired product elutes 65-75% EtOAc/Hex to provide (1R,3S)-3-(5-((2-bromopyrazolo[1,5-a]pyrazin-4-yl)amino)-1-(tert-butyl)-1H-pyrazol-3-yl)cyclopentyl isopropylcarbamate (407 mg, 72% yield).

### Step 2: Synthesis of (1R,3S)-3-(5-((2-(2-(1,3-dioxolan-2-yl)-3-((4-methoxybenzyl)oxy)phenyl) pyrazolo[1,5-a]pyrazin-4-yl)amino)-1-(tert-butyl)-1H-pyrazol-3-yl)cyclopentyl isopropyl-carbamate

A 40 mL reaction vial was charged with (1R,3S)-3-(5-((2-bromopyrazolo[1,5-a]pyrazin-4-yl)amino)-1-(tert-butyl)-1H-pyrazol-3-yl)cyclopentyl isopropylcarbamate (150 mg, 0.3 mmol, 1 equiv), 2-(2-(1,3-dioxolan-2-yl)-3-((4-methoxybenzyl)oxy)phenyl)-4,4,5,5-tetramethyl-1,3,2-dioxaborolane (159 mg, 0.39 mmol, 1.3 equiv), PdDPPF (19 mg, 0.03 mmol, 0.1 equiv), potassium phosphate (189 mg, 0.89 mmol, 3 equiv), dioxane (5 mL) and water (1 mL). Nitrogen gas was bubbled through the reaction mixture for 5 min, the reaction vessel was sealed and heated at 50° C. for 3 h under an atmosphere of nitrogen. The crude reaction was diluted with 5 mL water. The reaction mixture was extracted with EtOAc (3×10 mL), dried over sodium sulphate, filtered, and concentrated to dryness. The crude material was purified by silica gel column chromatography, 0 to 100% EtOAc/Hex, desired product elutes 100% EtOAc/Hex to afford (1R,3S)-3-(5-((2-(2-(1,3-dioxolan-2-yl)-3-((4-methoxybenzyl)oxy)phenyl)pyrazolo[1,5-a]pyrazin-4-yl)amino)-1-(tert-butyl)-1H-pyrazol-3-yl)cyclopentyl isopropylcarbamate (79 mg, 37% yield)

Step 3: Synthesis of (1R,3S)-3-(3-((2-(2-formyl-3-hydroxyphenyl)pyrazolo[1,5-a]pyrazin-4-yl)amino)-1H-pyrazol-5-yl)cyclopentyl isopropylcarbamate

A 15 mL reaction vial was charged with (1R,3S)-3-(5-((2-(2-(1,3-dioxolan-2-yl)-3-((4-methoxybenzyl)oxy)phenyl)pyrazolo[1,5-a]pyrazin-4-yl)amino)-1-(tert-butyl)-1H-pyrazol-3-yl)cyclopentyl isopropylcarbamate (78 mg, 0.11 mmol, 1 equivalent) and 2 mL formic acid. The reaction mixture was heated at 75° C. for 5 h. The reaction was then let cool to room temperature, was diluted with 1 mL water and purified by preparative HPLC, 10 to 70% MeCN/H₂O with 0.05% TFA modifier to afford (1R,3S)-3-(3-((2-(2-formyl-3-hydroxyphenyl)pyrazolo[1,5-a]pyrazin-4-yl)amino)-1H-pyrazol-5-yl)cyclopentyl isopropylcarbamate (28 mg, 52% yield) as a white solid. ¹H NMR (400 MHz, DMSO) δ 11.74 (s, 1H), 10.35 (s, 1H), 8.25 (d, J=5.0 Hz, 1H), 7.75-7.67 (m, 2H), 7.52 (d, J=5.0 Hz, 1H), 7.26 (dd, J=7.6, 1.1 Hz, 1H), 7.10 (d, J=8.4 Hz, 1H), 6.95 (d, J=7.7 Hz, 1H), 6.51 (s, 1H), 5.02 (s, 1H), 3.21-3.04 (m, 1H), 2.12-2.02 (m, 1H), 2.01-1.85 (m, 1H), 1.85-1.56 (m, 3H), 1.03 (d, J=6.6 Hz, 6H). MS (ESI) calcd. for C₂₆H₂₈N₆O₄, 489.21 m/z, found [M+H]⁺ 490.300 m/z.

### Example 17: Synthesis of Compound 75: (1R,3S)-3-[5-({4-[2-(2-formyl-3-hydroxyphenyl)ethynyl]pyridin-2-yl}amino)-2H-pyrazol-3-yl]cyclopentyl N-isopropylcarbamate; trifluoroacetic acid

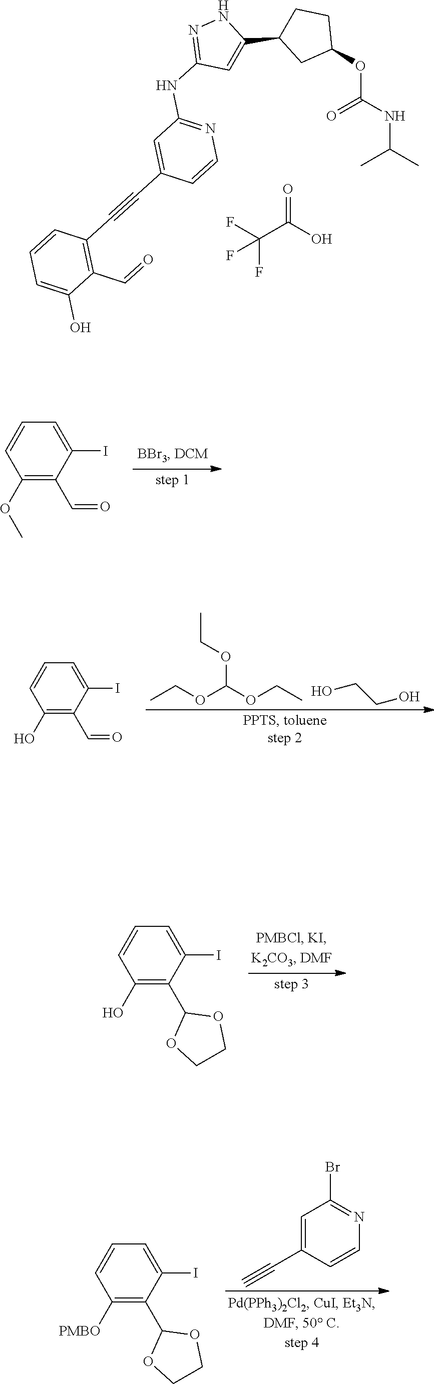

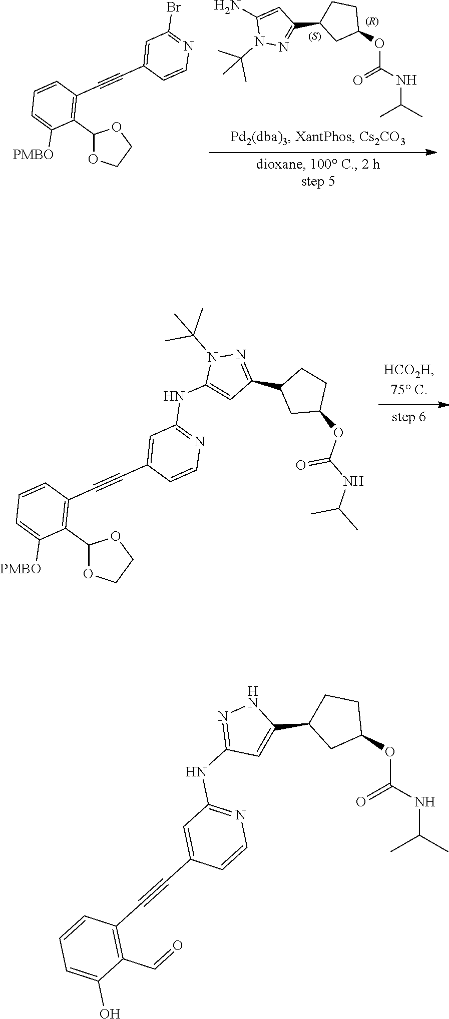

### Step 1: Synthesis of 2-hydroxy-6-iodobenzaldehyde

A solution of 2-iodo-6-methoxybenzaldehyde (16.2 g, 61.821 mmol, 1 equiv) in DCM (40 mL) was cooled to −70° C. and let stir for 5 min under nitrogen atmosphere followed by the addition of BBr₃ (80.37 mL, 80.367 mmol, 1.3 equiv) in dropwise at −70° C. The resulting mixture was stirred for 1 h at −70° C. under nitrogen atmosphere. The resulting mixture was quenched with water dropwise at 0° C., then extracted with DCM (30 mL×4). The combined organic layers were washed with H₂O (50 mL×2), dried over anhydrous Na₂SO₄. After filtration, the filtrate was concentrated under reduced pressure. The residue was purified by silica gel column with EA/PE (0-30%) to afford 2-hydroxy-6-iodobenzaldehyde (13.9 g, 81.77%) as a yellow solid. MS (ESI) calcd. for C₇H₅IO₂, 248.1 m/z, found 249.1 [M+H]⁺.

### Step 2: Synthesis of 2-(1,3-dioxolan-2-yl)-3-iodophenol

To a stirred mixture of 2-hydroxy-6-iodobenzaldehyde (7 g, 28.224 mmol, 1 equiv) and ethylene glycol (10.51 g, 169.344 mmol, 6 equiv) and PPTS (709.26 mg, 2.822 mmol, 0.1 equiv) in toluene (30 mL) was added triethyl orthoformate (12.55 g, 84.672 mmol, 3 equiv) in portions at 30° C. The resulting mixture was stirred for 2 h at 90° C. The resulting mixture was cooled to room temperature, diluted with water, and extracted with EA (3×200 mL). The combined organic layers were washed with brine (3×200 mL), dried over anhydrous Na₂SO₄ and concentrated under reduced pressure. The residue was purified by silica gel column chromatography, eluted with EA:PE (1:3) to afford 2-(1,3-dioxolan-2-yl)-3-iodophenol (5.1 g, 54.57%) as a yellow solid. MS (ESI) calcd. for C₉H₉IO₃, 292.1 m/z, found 293.1 [M+H].

### Step 3: Synthesis of 2-{2-iodo-6-[(4-methoxyphenyl)methoxy]phenyl}-1,3-dioxolane

A solution of 2-(1,3-dioxolan-2-yl)-3-iodophenol (5.1 g, 17.461 mmol, 1 equiv), KI (2.90 g, 17.461 mmol, 1 equiv) and K₂CO₃ (4.83 g, 34.922 mmol, 2 equiv) and PMBCl (3.28 g, 20.953 mmol, 1.2 equiv) in DMF (25 mL) was stirred for 2 h at 80° C. under N₂ atmosphere. The resulting mixture was cooled to room temperature, diluted with water and extracted with EA (100 mL×4). The combined organic layers were washed with H₂O (100 mL×3), dried over anhydrous Na₂SO₄. After filtration, the filtrate was concentrated under reduced pressure. The residue was purified by silica gel column with PE:EA (1:1) to afford 2-{2-iodo-6-[(4-methoxyphenyl)methoxy]phenyl}-1,3-dioxolane (6.7 g, 87.59%) as a white solid. MS (ESI) calcd. for C₁₇H₁₇IO₄, 412.2 m/z, found 413.2 [M+H]+.

### Step 4: Synthesis of 2-bromo-4-{2-[2-(1,3-dioxolan-2-yl)-3-[(4-methoxyphenyl)methoxy]phenyl]ethynyl}pyridine

To a stirred solution of 2-bromo-4-ethynylpyridine (88.0 mg, 0.483 mmol, 1 equiv) and 2-{2-iodo-6-[(4-methoxyphenyl)methoxy]phenyl}-1,3-dioxolane (199.3 mg, 0.483 mmol, 1 equiv) in DMF (5 mL, 64.608 mmol) were added Pd(PPh₃)₂Cl₂ (50.9 mg, 0.072 mmol, 0.15 equiv), CuI (18.4 mg, 0.097 mmol, 0.2 equiv) and Et₃N (146.8 mg, 1.449 mmol, 3 equiv) at room temperature. The resulting mixture was stirred for 1 h at 50° C. then cooled to room temperature and diluted with water. The resulting mixture was extracted with EA (3×20 mL). The combined organic layers were washed with brine (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O (10 mM NH₄HCO₃) in ACN, 10% to 100% gradient in 50 min; detector, UV 254 nm. This resulted in 2-bromo-4-{2-[2-(1,3-dioxolan-2-yl)-3-[(4-methoxyphenyl)methoxy]phenyl]ethynyl}pyridine (80.0 mg, 35.48%) as a yellow oil. MS (ESI) calcd. for C₂₄H₂₀BrNO₄, 465.06 m/z, found 466.10 [M+H]⁺.

### Step 5: Synthesis of (1R,3S)-3-{1-tert-butyl-5-[(4-{2-[2-(1,3-dioxolan-2-yl)-3-[(4-methoxyphenyl)methoxy]phenyl]ethynyl}pyridin-2-yl)amino]pyrazol-3-yl}cyclopentyl N-isopropylcarbamate

To a stirred mixture of 2-bromo-4-{2-[2-(1,3-dioxolan-2-yl)-3-[(4-methoxyphenyl)methoxy]phenyl]ethynyl}pyridine (70.0 mg, 0.150 mmol, 1 equiv) and (1R,3S)-3-(5-amino-1-tert-butylpyrazol-3-yl)cyclopentyl N-isopropylcarbamate (46.3 mg, 0.150 mmol, 1 equiv) in dioxane (5 mL) were added Pd₂(dba)₃ (20.6 mg, 0.022 mmol, 0.15 equiv) and XantPhos (13.0 mg, 0.022 mmol, 0.15 equiv) at room temperature under N₂ atmosphere. The resulting mixture was stirred for 1 h at 100° C. under N₂ atmosphere then cooled to room temperature and diluted with water. The resulting mixture was extracted with EA (3×20 mL). The combined organic layers were washed with brine (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, H₂O (10 mM NH₄HCO₃) in ACN, 10% to 100% gradient in 60 min; detector, UV 254 nm. This resulted in (1R,3S)-3-{1-tert-butyl-5-[(4-{2-[2-(1,3-dioxolan-2-yl)-3-[(4-methoxyphenyl)methoxy]phenyl]ethynyl}pyridin-2-yl)amino]pyrazol-3-yl}cyclopentyl N-isopropylcarbamate (30.0 mg, 28.80%) as a yellow solid. MS (ESI) calcd. for C₄₀H₄₇N₅O₆, 693.35 m/z, found 694.40 [M+H]⁺.

### Step 6: Synthesis of (1R,3S)-3-[5-({4-[2-(2-formyl-3-hydroxyphenyl)ethynyl]pyridin-2-yl}amino)-2H-pyrazol-3-yl]cyclopentyl N-isopropylcarbamate; trifluoroacetic acid

A solution of (1R,3S)-3-{1-tert-butyl-5-[(4-{2-[2-(1,3-dioxolan-2-yl)-3-[(4-methoxyphenyl) methoxy]phenyl]ethynyl}pyridin-2-yl)amino]pyrazol-3-yl}cyclopentyl N-isopropylcarbamate (30.0 mg, 0.043 mmol, 1 equiv) in formic acid (3 mL) was stirred for 2 h at 75° C. The resulting mixture was concentrated under reduced pressure. The crude product was purified by Prep-HPLC with the following conditions (Column: Xselect CSH Prep C18 OBD Column, 30×150 mm, 5 um; Mobile Phase A: Water (0.1% FA), Mobile Phase B: ACN; Flow rate: 60 mL/min; Gradient: 19% B to 41% B in 10 min; Wave Length: 254 nm/220 nm) to afford (1R,3S)-3-[5-({4-[2-(2-formyl-3-hydroxyphenyl)ethynyl]pyridin-2-yl}amino)-2H-pyrazol-3-yl]cyclopentyl N-isopropylcarbamate; trifluoroacetic acid (2.8 mg, 11.00%) as a yellow solid. MS (ESI) calcd. for C₂₈H₂₈F₃N₅O₆, 473.21 m/z, found 474.20 [M+H]⁺. ¹H NMR (400 MHz, DMSO-d₆) δ (ppm): 11.45 (br s, 1H), 10.58 (br s, 1H), 10.29 (s, 1H), 8.21 (d, J=6.0 Hz, 1H), 7.89 (s, 1H), 7.75-7.78 (m, 1H), 7.39 (s, 1H), 7.11 (d, J=8.8 Hz, 1H), 6.95-7.01 (m, 2H), 6.04 (s, 1H), 5.01-5.02 (m, 1H), 3.51-3.61 (m, 1H), 3.10-3.14 (m, 1H), 2.46-2.47 (m, 1H), 2.03-2.09 (m, 1H), 1.90-1.96 (m, 1H), 174-1.76 (m, 2H), 1.60-1.67 (m, 1H), 1.04 (d, J=6.4 Hz, 6H). ¹⁹F NMR (376 MHz, DMSO-d₆) δ (ppm): −74.20.

### Example 18: Synthesis of Compound 9: (1R,3S)-3-{5-[(1R,2R)-2-(2-formyl-3-hydroxyphenyl) cyclopropaneamido]-2H-pyrazol-3-yl}cyclopentyl N-isopropylcarbamate

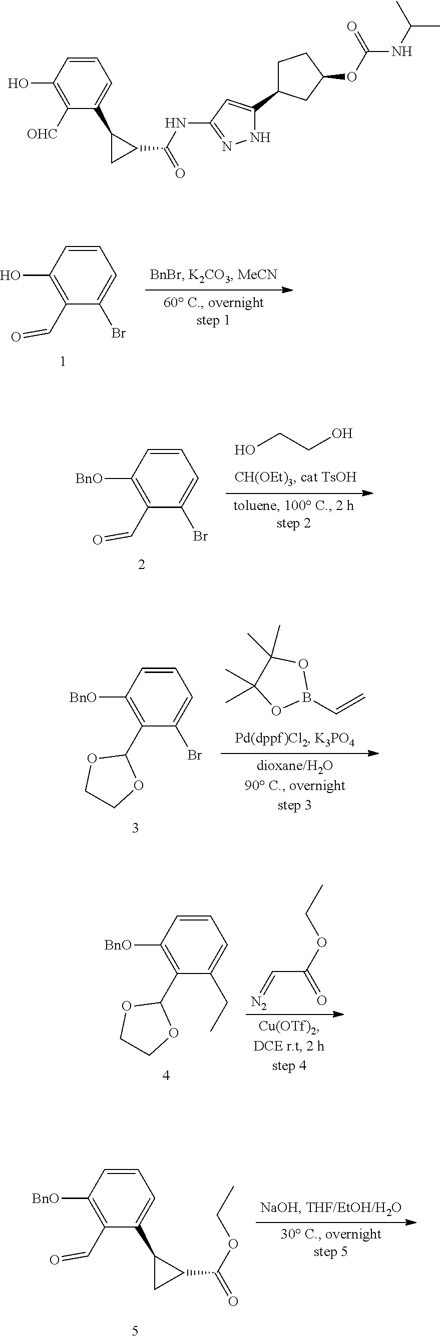

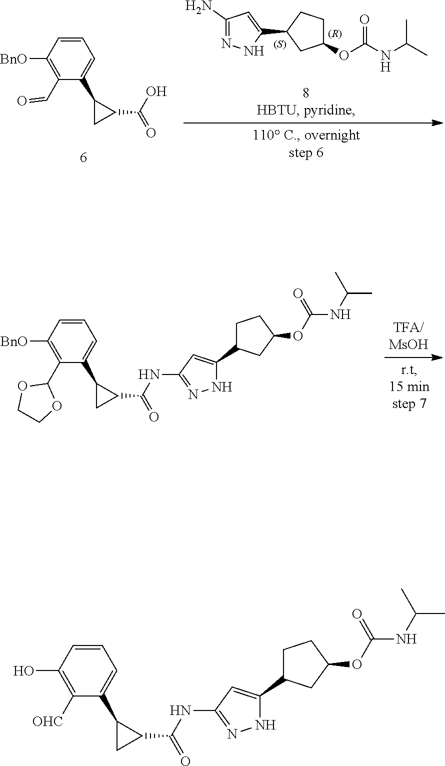

### Step 1: Synthesis of 2-(benzyloxy)-6-bromobenzaldehyde

To a 500 mL round-bottomed flask equipped with a stirring bar was added 2-bromo-6-hydroxybenzaldehyde (10 g, 49.747 mmol, 1 equiv) in ACN (100 mL). BnBr (12.8 g, 74.620 mmol, 1.5 equiv) and K₂CO₃ (687.5 mg, 4.975 mmol, 0.1 equiv) were added to the reaction flask. The resulting mixture was stirred at 60° C. overnight. The reaction mixture was then treated with H₂O (50 mL), extracted with EtOAc (100 mL×3). The combined organic layers were washed with brine (50 mL), dried over anhydrous Na₂SO₄. After filtration, the filtrate was concentrated under reduced pressure. The residue was purified by silica gel column chromatography (0-50% PE/EA) to afford 2-(benzyloxy)-6-bromobenzaldehyde (13 g, 89.7%) as a yellow solid.

### Step 2: Synthesis of 2-[2-(benzyloxy)-6-bromophenyl]-1,3-dioxolane

2-(Benzyloxy)-6-bromobenzaldehyde (13 g, 44.651 mmol, 1 equiv) in toluene (130 mL) were added to a 500 mL round-bottom flask and stirred until homogeneous. Ethylene glycol (14.94 mL, 267.906 mmol, 6 equiv), triethyl orthoformate (19.9 g, 133.953 mmol, 3 equiv) and p-toluenesulfonic acid (0.8 g, 4.465 mmol, 0.1 equiv) was added and the reaction mixture was stirred overnight at 100° C. The reaction mixture was then treated with H₂O (50 mL), extracted with EA (100 mL×3). The combined organic layers were washed with brine (50 mL), dried over anhydrous Na₂SO₄, filtered and concentrated under reduced pressure. The residue was purified by silica gel column chromatography (0-50% PE/EA) to afford 2-(benzyloxy)-6-bromobenzaldehyde-1,3-dioxolane (13 g, 89.7%) as a yellow solid. MS (ESI), calcd. for C₁₆H₁₅BrO₃, 334.02 m/z, found 335.95 [M+H]⁺.

### Step 3: Synthesis of 2-[2-(benzyloxy)-6-ethenylphenyl]-1,3-dioxolane

2-[2-(Benzyloxy)-6-bromophenyl]-1,3-dioxolane (5 g, 14.917 mmol, 1 equiv), 2-ethenyl-4,4,5,5-tetramethyl-1,3,2-dioxaborolane (2.7 g, 17.900 mmol, 1.2 equiv), 1,4-dioxane (50 mL) and H₂O (10 mL) were added to a 500 mL round-bottom flask and stirred until homogeneous. K₃PO₄ (9.50 g, 44.751 mmol, 3 equiv), Pd(dppf)Cl₂ (1.2 g, 1.492 mmol, 0.1 equiv) were added to the reaction flask at rt. The resulting mixture was stirred at 90° C. for 2 h under N₂. The reaction progress was monitored by LCMS. After cooling down to room temperature, the reaction mixture was treated with H₂O (50 mL) and extracted with EA (100 mL×3). The combined organic extracts were washed with brine (50 mL), dried over anhydrous Na₂SO₄ and concentrated under vacuum to yield the crude product. The residue was purified by silica gel column chromatography (0-20% PE/EA) to afford 2-[2-(benzyloxy)-6-ethenylphenyl]-1,3-dioxolane (2 g, 47.4%) as a white solid. MS (ESI), calcd. for C₁₈H₁₈O₃, 282.13 m/z, found 283.05 [M+H]⁺.

### Step 4: Synthesis of ethyl (1R,2R)-2-[3-(benzyloxy)-2-(1,3-dioxolan-2-yl)phenyl]cyclo-propane-1-carboxylate

Into a 40 mL vessel were added 2-[2-(benzyloxy)-6-ethenylphenyl]-1,3-dioxolane (1 g, 3.542 mmol, 1 equiv), DCE (10 mL), ethyl diazoacetate (606.2 mg, 5.313 mmol, 1.5 equiv) and Cu(OTf)₂ (384.3 mg, 1.063 mmol, 0.3 equiv) and the resulting mixture was stirred for 2 h at room temperature. The reaction mixture was then treated with H₂O (20 mL), extracted with DCM (20 mL×3), and the combined extracts were washed with brine (20 mL), dried over anhydrous Na₂SO₄, filtered, and concentrated to dryness in vacuum to give a nearly yellow viscous oil. The oil was then subjected to silica gel chromatography (0-50% PE/EA) to afford ethyl (1R,2R)-2-[3-(benzyloxy)-2-formylphenyl]cyclopropane-1-carboxylate (200 mg, 17.41%) as a light yellow solid. MS (ESI), calcd. for C₂₀H₂₀O₄, 324.14 m/z, found 325.05 [M+H]⁺.

### Step 5: Synthesis of (1R,2R)-2-[3-(benzyloxy)-2-(1,3-dioxolan-2-yl)phenyl]cyclopropane-1-carboxylic acid

Into a 40 mL vessel were added ethyl (1R,2R)-2-[3-(benzyloxy)-2-(1,3-dioxolan-2-yl)phenyl]cyclopropane-1-carboxylate (200 mg, 0.543 mmol, 1 equiv), NaOH (1 mL, 2 mmol, 2 mol/L) and EtOH (1 mL, 17.213 mmol, 31.71 equiv) and the resulting mixture was stirred at 30° C. overnight. The resulting mixture was concentrated, and the residue was diluted with H₂O (5 mL). The mixture was acidified to pH 6 with HCl (2 mol/L) and extracted with EA (5 mL×3). The combined organic layers were washed with brine (5 mL), dried over anhydrous Na₂SO₄, filtrated and concentrated under reduced pressure to afford (1R,2R)-2-[3-(benzyloxy)-2-(1,3-dioxolan-2-yl)phenyl]cyclopropane-1-carboxylic acid (150 mg, 81.1%) as a yellow solid. MS (ESI), calcd. for C₂₀H₂₀O₅, 340.13 m/z, found 340.80[M+H]⁺.

### Step 6: Synthesis of (1R,3S)-3-{5-[(1R,2R)-2-[3-(benzyloxy)-2-formylphenyl]cyclopropane-amido]-2H-pyrazol-3-yl}cyclopentyl N-isopropylcarbamate

Into a 8 mL vessel were added ethyl (1R,2R)-2-[3-(benzyloxy)-2-formylphenyl]cyclopropane-1-carboxylate (96.4 mg, 0.297 mmol, 1.5 equiv), pyridine (1 mL), (1R,3S)-3-(5-amino-2H-pyrazol-3-yl)cyclopentyl N-isopropylcarbamate (50 mg, 0.198 mmol, 1.00 equiv) and HBTU (75.2 mg, 0.297 mmol, 1.5 equiv). The resulting mixture was stirred at 110° C. overnight. The resulting mixture was diluted with H₂O (5 mL) and extracted with EA (5 mL×3). The combined organic layers were washed with brine (5 mL), dried over anhydrous Na₂SO₄, filtrated and concentrated under reduced pressure. The residue was purified by TLC (DCM:MeOH=15:1) to afford (1R,3S)-3-{5-[(1R,2R)-2-[3-(benzyloxy)-2-formylphenyl]cyclopropaneamido]-2H-pyrazol-3-yl}cyclopentyl N-isopropylcarbamate (45 mg, 42.8%) as a yellow solid. MS (ESI), calcd. for C₃₀H₃₄N₄O₅, 530.25 m/z, found 531.25[M+H]⁺.

### Step 7: Synthesis of (1R,3S)-3-{5-[(1R,2R)-2-(2-formyl-3-hydroxyphenyl)cyclopropane-amido]-2H-pyrazol-3-yl}cyclopentyl N-isopropylcarbamate

Into a 50 mL vessel were added (1R,3S)-3-{5-[(1R,2R)-2-[3-(benzyloxy)-2-formylphenyl]cyclo-propaneamido]-2H-pyrazol-3-yl}cyclopentyl N-isopropylcarbamate (35 mg, 0.066 mmol, 1 equiv), methanesulfonic acid (0.5 mL) and TFA (2.5 mL). The resulting mixture was stirred at room temperature for 15 min. After filtration, the filtrate was concentrated under reduced pressure to afford the yellow oil. The yellow oil was purified by Prep-HPLC with the following conditions (Column: XBridge Shield RP 18 OBD Column, 30×150 mm, 5 μm; Mobile Phase A: Water (0.05% TFA), Mobile Phase B: ACN; Flow rate: 60 mL/min; Gradient: 28% B to 58% B in 7 min; Wave Length: 254 nm; RT1 (min): 6.58) to afford (1R,3S)-3-{5-[(1S,2R)-2-(2-formyl-3-hydroxyphenyl)cyclopropaneamido]-2H-pyrazol-3-yl}cyclopentyl N-isopropylcarbamate (1.7 mg, 5.6%) as a yellow solid. MS (ESI) calcd. H₁ for C₂₃H₂₈N₄O₅, 440.21 m/z, found 441.20 [M+H]⁺. ¹H NMR (300 MHz, DMSO-d₆) δ 11.37 (s, 1H), 10.59 (s, 1H), 10.47 (s, 1H), 7.47 (t, J=8.0 Hz, 1H), 6.91-7.00 (m, 1H), 6.87 (d, J=8.3 Hz, 1H), 6.75 (d, J=7.6 Hz, 1H), 6.34 (s, 1H), 5.00 (s, 1H), 3.05 (t, J=8.6 Hz, 2H), 2.12 (dd, J=8.1, 4.6 Hz, 1H), 2.01 (d, J=7.9 Hz, 3H), 1.89 (s, 2H), 1.68-1.74 (m, 2H), 1.39-1.48 (m, 1H), 1.30-1.36 (m, 1H), 1.24 (s, 1H), 1.04 (d, J=6.5 Hz, 6H)

### Example 19: Synthesis of Compound 58: (1R,3S)-3-(3-(3-(2-formyl-3-hydroxyphenyl)isothiazole-5-carboxamido)-1H-pyrazol-5-yl)cyclopentyl isopropylcarbamate

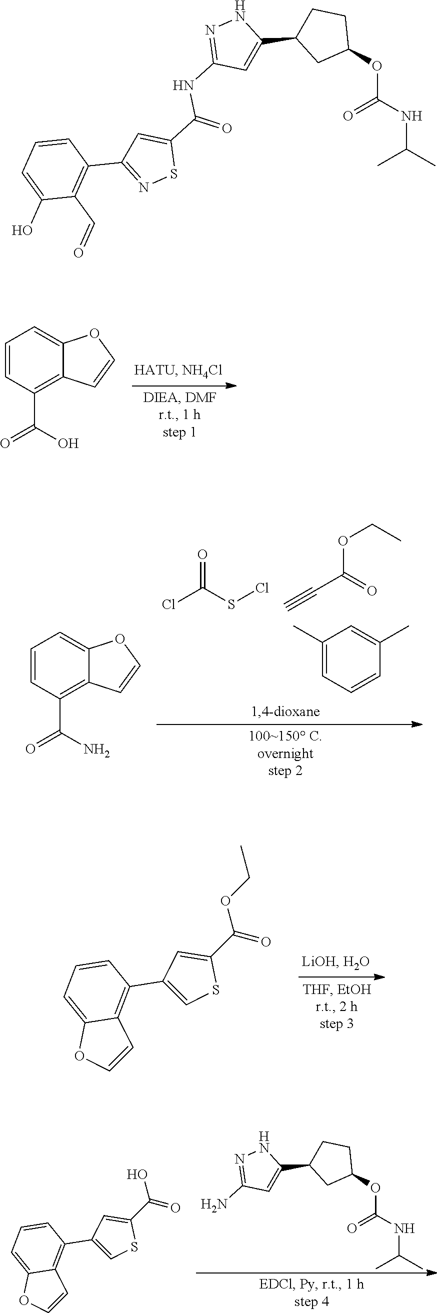

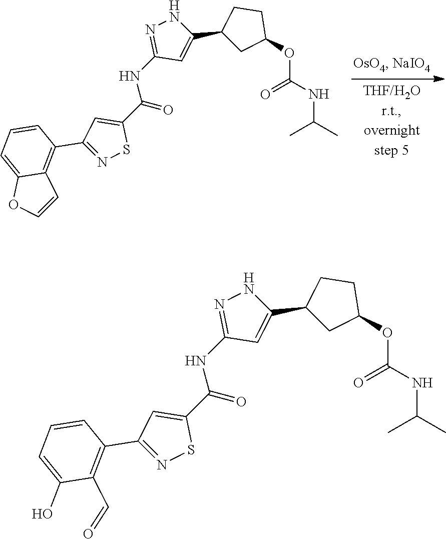 [SMILES: CC(C)NC(=O)O[C@@H]1CC[C@H](c2cc(NC(=O)c3cc(-c4cccc5occc45)ns3)n[nH]2)C1 ; CC(C)NC(=O)O[C@@H]1CC[C@H](c2cc(NC(=O)c3cc(-c4cccc(O)c4C=O)ns3)n[nH]2)C1]

### Step 1: Synthesis of benzofuran-4-carboxamide

1-Benzofuran-4-carboxylic acid (2.0 g, 12.335 mmol), NH₄Cl (1.3 g, 24.670 mmol), DIEA (4.8 g, 37.005 mmol), a stir bar and DMF (20 mL) were added to a 100 mL flask and stirred until homogenous, then treated with HATU (5.2 g, 13.569 mmol). The resulting mixture was stirred at rt. for 1 h. The reaction was quenched with H₂O and extracted with EA. The organic layers were combined, washed with brine, dried over Na₂SO₄, filtered and concentrated. The residue obtained was purified by silica gel chromatography (PE/EA=0-66%) to afford 1-benzofuran-4-carboxamide (1.9 g, 95.58%) as a white solid. MS (ESI) calcd. for C₉H₇NO₂, 161.05 m/z, found: 162.05 [M+H]⁺.

### Step 2: Synthesis of ethyl 3-(benzofuran-4-yl)isothiazole-5-carboxylate

1-Benzofuran-4-carboxamide (1.8 g, 11.169 mmol), chloro(chlorosulfanyl)methanone (2.2 g, 16.754 mmol), a stir bar and 1,4-dioxane (40 mL) were added to a 250 mL flask. The resulting mixture was stirred at 100° C. overnight and then quenched with H₂O and extracted with EA. The organic layers were combined, washed with brine, dried over Na₂SO₄, filtered and concentrated. The residue obtained was purified by silica gel chromatography (PE/EA=0-5%) to afford 5-(benzofuran-4-yl)-1,3,4-oxathiazol-2-one (Int 1) as a yellow solid (740.0 mg). The yellow solid was then transferred to a 40 mL microwave tube along with ethyl propiolate (1.3 g, 13.252 mmol) and suspended in M-xylene (15 mL). The mixture was stirred at 150° C. overnight, then concentrated. The residue obtained was purified by silica gel chromatography (PE/EA=0-10%) to afford ethyl 3-(1-benzofuran-4-yl)-1,2-thiazole-5-carboxylate (390.0 mg, 12.78%) as a yellow solid. MS (ESI) calcd. for C₁₄H₁₁NO₃S, 273.05 m/z, found: 274.05 [M+H]⁺.

### Step 3: Synthesis of 3-(benzofuran-4-yl)isothiazole-5-carboxylic acid

Ethyl 3-(1-benzofuran-4-yl)-1,2-thiazole-5-carboxylate (370.0 mg, 1.354 mmol), EtOH (2 mL), a stir bar and THF (6 mL) were added to a 100 mL flask and stirred until homogenous, then treated with lithium hydroxide (1.35 mL, 4.062 mmol, 3 M). The resulting mixture was stirred at rt. for 2 h. The reaction was concentrated and diluted with H₂O. The pH of the aqueous phase was adjusted to 3-4 used HCl solution (2 M). The mixture was extracted with EA. The organic layers were combined, dried over Na₂SO₄, filtered and concentrated to afford 3-(1-benzofuran-4-yl)-1,2-thiazole-5-carboxylic acid (280.0 mg, 84.33%) as a yellow solid. MS (ESI) calcd. for C₁₂H₇NO₃S, 245.01, found, 246.00 [M+H]⁺.

### Step 4: Synthesis of (1R,3S)-3-(3-(3-(benzofuran-4-yl)isothiazole-5-carboxamido)-1H-pyrazol-5-yl)cyclopentyl isopropylcarbamate

(1R,3S)-3-(5-amino-2H-pyrazol-3-yl)cyclopentyl N-isopropylcarbamate (100.0 mg, 0.396 mmol), 3-(1-benzofuran-4-yl)-1,2-thiazole-5-carboxylic acid (116.0 mg, 0.473 mmol), a stir bar and pyridine (1 mL) were added to a 8 mL vial and stirred until homogenous, then treated with EDCI (152.0 mg, 0.793 mmol). The resulting mixture was stirred at rt. for 1 h, then concentrated. The residue obtained was purified by reverse phase chromatography; MeCN/H₂O (10 mM NH₄HCO₃)=5-60%, to afford (1R,3S)-3-{5-[3-(1-benzofuran-4-yl)-1,2-thiazole-5-amido]-2H-pyrazol-3-yl}cyclopentyl N-isopropylcarbamate (130.0 mg, 68.40%) as a yellow solid. MS (ESI) calcd. for C₂₄H₂₅N₅O₄S, 479.16, found, 480.05 [M+H]⁺.

### Step 5: Synthesis of (1R,3S)-3-(3-(3-(2-formyl-3-hydroxyphenyl)isothiazole-5-carboxamido)-1H-pyrazol-5-yl)cyclopentyl isopropylcarbamate

(1R,3S)-3-{5-[3-(1-benzofuran-4-yl)-1,2-thiazole-5-amido]-2H-pyrazol-3-yl}cyclopentyl N-isopropylcarbamate (120.0 mg, 0.250 mmol), a stir bar, THF (3 mL) and H₂O (3 mL) were added to a 100 mL flask and stirred until homogenous, then treated with osmium tetroxide (254.0 mg, 0.025 mmol, 2.5% in t-BuOH) at 0° C. After stirring for 30 min at rt, NaIO₄ (535 mg, 2.501 mmol) was added in several portions. The resulting mixture was stirred at rt. overnight. The reaction was quenched with H₂O and extracted with EA. The organic layers were combined, dried over Na₂SO₄, filtered and concentrated. The residue obtained was purified by Prep-HPLC (Column: XBridge Prep OBD C18 Column, 30×150 mm, 5 μm; Mobile Phase A: Water (0.05% TFA), Mobile Phase B: ACN; Flow rate: 60 mL/min; Gradient: 38% B to 60% B in 10 min; Wave Length: 254/220 nm; RT1 (min): 8.6) to afford (1R,3S)-3-{5-[3-(2-formyl-3-hydroxyphenyl)-1,2-thiazole-5-amido]-2H-pyrazol-3-yl}cyclopentyl N-isopropylcarbamate (10.9 mg, 8.89%) as an off-white solid. MS (ESI) calcd. for C₂₃H₂₅N₅O₅S, 483.16, found, 484.25 [M+H]⁺. ¹H NMR (400 MHz, DMSO-d₆) δ 11.62 (s, 1H), 11.34 (s, 1H), 10.27 (s, 1H), 8.51 (s, 1H), 7.71 (t, J=8.0 Hz, 1H), 7.23 (dd, J=7.6, 1.0 Hz, 1H), 7.14 (d, J=8.3 Hz, 1H), 7.01-6.88 (m, 1H), 6.46 (s, 1H), 5.02 (s, 1H), 3.68-3.52 (m, 1H), 3.19-3.03 (m, 1H), 2.46-2.50 (m, 1H), 2.05 (q, J=8.7 Hz, 1H), 2.00-1.84 (m, 1H), 1.82-1.55 (m, 3H), 1.04 (d, J=6.5 Hz, 6H).

### Example 20: Synthesis of Selected Compounds 115-259

The compounds shown in Table 1B, were synthesized according to the procedures of Examples 1 to 19 or analogously to these procedures.

TABLE 1B
Characterization Data for selected compounds

| # | Compound | Characterization Data |
|---|---|---|
| 115 | 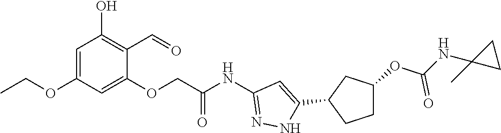 [SMILES: CCOc1cc(O)c(C=O)c(OCC(=O)Nc2cc([C@H]3CC[C@@H](OC(=O)NC4(C)CC4)C3)[nH]n2)c1] | ¹H NMR (400 MHz, DMSO-D6) δ 12.33 (s, 1H), 10.59-10.54 (m, 1H), 10.16-10.09 (m, 1H), 7.42- 7.29 (m, 1H), 6.34-6.27 (m, 1H), 6.17-6.08 (m, 1H), 6.06-6.01 (m, 1H), 5.09-4.96 (m, 1H), 4.86-4.76 (m, 2H), 4.12-4.06 (m, 2H), 3.10- 2.96 (m, 1H), 2.48-2.40 (m, 1H), |
| | | 2.06-1.87 (m, 2H), 1.75-1.53 (m, |
| | | 3H), 1.31 (t, J = 7.0 Hz, 3H), 1.26- |
| | | 1.17 (m, 3H), 0.60-0.52 (m, 2H), |
| | | 0.50-0.42 (m, 2H). MS (ESI) mass |
| | | calcd. for C₂₄H₃₀N₄O₇ 486.21 m/z, |
| | | found 487.35 [M + H]⁺ 487.35 m/z |
| 116 | 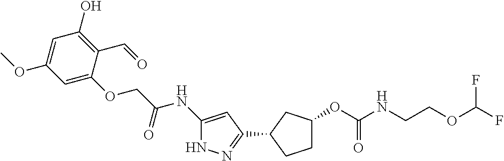 [SMILES: COc1cc(O)c(C=O)c(OCC(=O)Nc2cc([C@H]3CC[C@@H](OC(=O)NCCOC(F)F)C3)n[nH]2)c1] | ¹H NMR (400 MHz, DMSO-D6) δ 12.32 (s, 1H), 12.17 (s, 1H), 10.55 (s, 1H), 10.15 (s, 1H), 7.25 (t, J = 5.8 Hz, 1H), 6.87-6.43 (m, 1H), 6.31 (s, 1H), 6.14 (d, J = 2.2 Hz, 1H), 6.07 (d, J = 2.1 Hz, 1H), 5.03- 4.94 (m, 1H), 4.81 (s, 2H), 3.81 (s, 3H), 3.84-3.74 (m, 2H), 3.24-3.16 (m, 2H), 3.09-3.00 (m, 1H), 2.48- 2.41 (m, 1H), 2.04-1.97 (m, 1H), |
| | | 1.93-1.84 (m, 1H), 1.76-1.64 (m, |
| | | 2H), 1.64-1.53 (m, 1H). 19F NMR |
| | | (376 MHz, DMSO-D6) δ (ppm): |
| | | −82.708, MS (ESI) mass calcd. for |
| | | C₂₂H₂₆F₂N₄O₈ 512.17 m/z, |
| | | found [M + H]⁺ 513.15 m/z. |
| 117 | 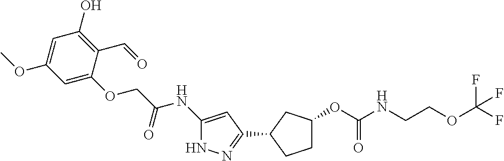 [SMILES: COc1cc(O)c(C=O)c(OCC(=O)Nc2cc([C@H]3CC[C@@H](OC(=O)NCCOC(F)(F)F)C3)n[nH]2)c1] | ¹H NMR (400 MHz, DMSO-D6) δ 12.32 (s, 1H), 12.18 (s, 1H), 10.55 (s, 1H), 10.15 (s, 1H), 7.35 (t, J = 5.9 Hz, 1H), 6.32 (s, 1H), 6.14 (d, J = 2.1 Hz, 1H), 6.07 (d, J = 2.2 Hz, 1H), 5.07-4.97 (m, 1H), 4.80 (s, 2H), 4.04 (t, J = 5.4 Hz, 2H), 3.81 (s, 3H), 3.30-3.23 (m, 2H), 3.10-3.00 (m, 1H), 2.48-2.43 (m, 1H), 2.08-1.96 (m, 1H), 1.95-1.81 |
| | | (m, 1H), 1.81-1.64 (m, 2H), 1.64- |
| | | 1.54 (m, 1H). MS (ESI) calcd. for |
| | | C₂₂H₂₅F₃N4O₈ 530.16 m/z, found |
| | | [M + H]⁺ 531.25 m/z. |
| 118 | 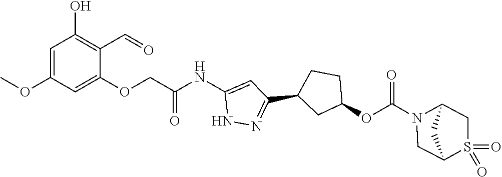 [SMILES: COc1cc(O)c(C=O)c(OCC(=O)Nc2cc([C@H]3CC[C@@H](OC(=O)N4C[C@H]5C[C@@H]4CS5(=O)=O)C3)n[nH]2)c1] | ¹H NMR (400 MHz, DMSO-D6) δ 12.32 (s, 1H), 10.56 (s, 1H), 10.15 (s, 1H), 6.31 (d, J = 10.3 Hz, 1H), 6.11 (dd, J = 27.3, 2.1 Hz, 2H), 5.07 (s, 1H), 4.81 (s, 2H), 4.54 (d, J = 18.6 Hz, 1H), 3.94 (s, 1H), 3.81 (s, 2H), 3.70-3.63 (m, 1H), 3.52- 3.44 (m, 1H), 3.25-3.00 (m, 3H), 2.49-2.44 (m, 1H), 2.44-2.37 (m, 2H), 2.35-2.28 (m, 1H), 2.10-1.95 (m, 1H), 1.95-1.84 (m, 1H), 1.83- |
| | | 1.68 (m, 3H). MS (ESI) mass |
| | | calcd. for C₂₄H₂₈N₄O₉S 548.16 |
| | | m/z, found, [M + H]⁺ 549.30 m/z. |
| 119 | 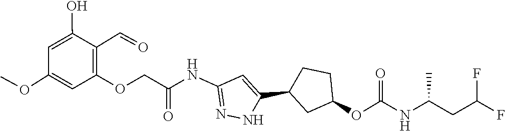 [SMILES: COc1cc(O)c(C=O)c(OCC(=O)Nc2cc([C@H]3CC[C@@H](OC(=O)N[C@H](C)CC(F)F)C3)[nH]n2)c1] | ¹H NMR (400 MHz, DMSO-D6) δ 12.30 (s, 1H), 12.18 (s, 1H), 10.55 (s, 1H), 10.15 (s, 1H), 7.14 (d, J = 8.3 Hz, 1H), 6.31 (s, 1H), 6.14 (d, J = 2.1 Hz, 1H), 6.09-5.86 (m, 2H), 5.02-4.96 (m, 1H), 4.81 (s, 2H), 3.81 (s, 3H), 3.72-3.62 (m, 1H), 3.11-3.00 (m, 1H), 2.50-2.41 (m, |
| | | 1H), 2.05-1.82 (m, 4H), 1.76-1.63 |
| | | (m, 2H), 1.63-1.53 (m, 1H), 1.07 |
| | | (d, J = 6.7 Hz, 3H). 19F NMR (376 |
| | | MHz, DMSO-D6) δ (ppm): |
| | | −114.780. MS (ESI) mass calcd. |
| | | for C₂₃H₂₈F₂N₄O₇ 510.19 m/z, |
| | | found [M + H]⁺ 511.30 m/z. |
| 120 | 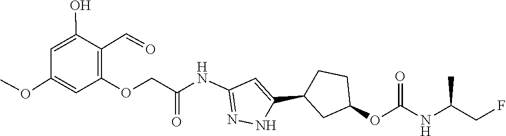 [SMILES: COc1cc(O)c(C=O)c(OCC(=O)Nc2cc([C@H]3CC[C@@H](OC(=O)N[C@@H](C)CF)C3)[nH]n2)c1] | ¹H NMR (400 MHz, DMSO-D6) δ 12.32 (s, 1H), 10.54 (s, 1H), 10.16 (s, 1H), 7.17 (d, J = 8.1 Hz, 1H), 6.31 (s, 1H), 6.15 (d, J = 2.1 Hz, 1H), 6.08 (d, J = 2.2 Hz, 1H), 5.02 (s, 1H), 4.81 (s, 2H), 4.31 (d, J = 5.6 Hz, 1H), 4.19 (d, J = 5.6 Hz, 1H), 3.82 (s, 3H), 3.80-3.66 (m, 1H), 3.16-2.97 (m, 1H), 2.50 (s, |
| | | 2H), 2.08-1.96 (m, 1H), 1.96- |
| | | 1.82 (m, 1H), 1.80-1.53 (m, 2H), |
| | | 1.05 (d, J = 6.9 Hz, 3H). MS (ESI) |
| | | calcd. for C₂₂H₂₇FN₄O₇ 478.19, |
| | | found [M + H]⁺ 479.30 m/z. |
| 121 | 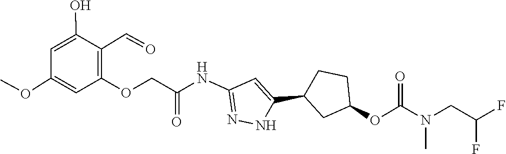 [SMILES: COc1cc(O)c(C=O)c(OCC(=O)Nc2cc([C@H]3CC[C@@H](OC(=O)N(C)CC(F)F)C3)[nH]n2)c1] | ¹H NMR (400 MHz, DMSO-D6) δ 12.33 (s, 1H), 12.19 (br s, 1H), 10.55 (s, 1H), 10.15 (s, 1H), 5.90- 6.36 (m, 4H), 5.00-5.08 (m, 1H), 4.80 (s, 2H), 3.81 (s, 3H), 3.50- 3.65 (m, 2H), 3.04-3.16 (m, 1H), 2.87 (s, 3H), 2.37-2.46 (m, 1H), 1.96-2.06 (m, 1H), 1.84-1.94 (m, 1H), 1.65-1.82 (m, 3H). MS (ESI) calcd. for C₂₂H₂₆F₂N₄O₇ 496.18 |
| | | m/z, found [M + H]⁺ 497.25 m/z. |
| 122 | 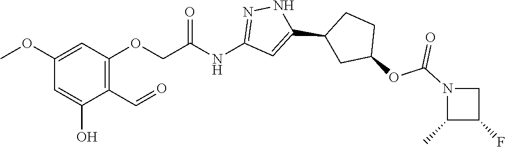 [SMILES: COc1cc(O)c(C=O)c(OCC(=O)Nc2cc([C@H]3CC[C@@H](OC(=O)N4C[C@H](F)[C@@H]4C)C3)[nH]n2)c1] | ¹H NMR (300 MHz, DMSO-D6) δ 12.33 (s, 1H), 10.57 (s, 1H), 10.15 (s, 1H), 6.32(s, 1H), 6.14 (s, 1H), 6.07 (s, 1H), 5.13-5.34 (m, 1H), 4.99-5.01 (m, 1H), 4.81(s, 2H), 4.39-4.48 (m, 1H), 4.05-4.17 (m, 1H), 3.81-3.85 (m, 4H), 2.41-2.52 (m, 1H), 1.98-2.03 (m, 1H), 1.81- 1.90 (m, 1H), 1.66-1.80 (m, 3H), |
| | | 1.10-1.30(m, 3H). 19F NMR (282 |
| | | MHz, DMSO-D6) δ −197.83, |
| | | −74.87. MS (ESI) calcd. for |
| | | C₂₃H₂₇FN₄O₇, 490.19 m/z, found |
| | | 491.15 [M + H]⁺ m/z. |
| 123 | 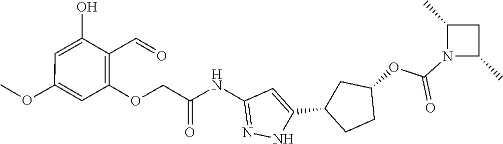 | ¹H NMR (400 MHz, DMSO-D6) δ 12.33 (s, 1H), 12.17 (s, 1H), 10.55 (s, 1H), 10.15 (s, 1H), 6.32 (s, 1H), 6.06-6.14 (m, 2H), 4.98-5.00 (m, 1H), 4.80 (s, 2H), 4.01-4.06 (m, 2H), 3.80 (s, 3H), 3.07-3.11 (m, 1H), 2.37-2.51 (m, 2H), 2.01-2.08 (m, 1H), 1.84-1.87 (m, 1H), 1.65- 1.76 (m, 3H), 1.32-1.34 (m, 1H), |
| | | 1.23-1.31 (m, 6H). MS (ESI) |
| | | calcd. for C₂₄H₃₀N₄O₇, 486.21 m/z, |
| | | found [M + H]⁺ 487.30 m/z. |
| 124 | 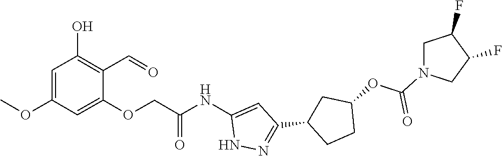 [SMILES: COc1cc(O)c(C=O)c(OCC(=O)Nc2cc([C@H]3CC[C@@H](OC(=O)N4C[C@@H](F)[C@H](F)C4)C3)n[nH]2)c1] | ¹H NMR (400 MHz, DMSO-D6) δ 12.32 (s, 1H), 12.18 (s, 1H), 10.55 (s, 1H), 10.15 (s, 1H), 6.35 (s, 1H), 6.14 (d, J = 2.0 Hz, 1H), 6.07 (d, J = 2.1 Hz, 1H), 5.35-5.28 (m, 1H), 5.27-5.18 (m, 1H), 5.11-5.01 (m, 1H), 4.80 (s, 2H), 3.63 (t, J = 2.7 Hz, 3H), 3.58-3.47 (m, 2H), 3.33 (d, J = 1.0 Hz, 2H), 3.11 (p, J = 8.2 Hz, 1H), 2.43-2.31 (m, 1H), 2.01- |
| | | 1.99 (m, 1H), 1.97-1.84 (m, 1H), |
| | | 1.84-1.66 (m, 3H). 19F NMR (377 |
| | | MHz, DMSO-D6) δ −188.65, |
| | | −189.02. MS (ESI) calcd. for |
| | | C₂₃H₂₆F₂N₄O₇, 508.18 m/z, found |
| | | [M + H]⁺ 509.20 m/z. |
| 125 | 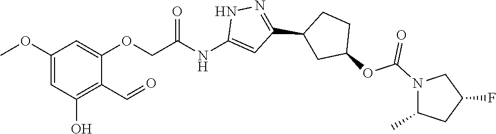 [SMILES: COc1cc(O)c(C=O)c(OCC(=O)Nc2cc([C@H]3CC[C@@H](OC(=O)N4C[C@H](F)C[C@@H]4C)C3)n[nH]2)c1] | ¹H NMR (400 MHz, DMSO-D6) δ 12.32 (s, 2H), 10.55 (s, 1H), 10.14 (s, 1H), 6.32 (s, 1H), 6.14 (d, J = 2.1 Hz, 1H), 6.06 (d, J = 2.2 Hz, 1H), 5.04 (s, 2H), 4.80 (s, 2H), 3.93 (s, 1H), 3.80 (s, 3H), 3.55 (d, J = 7.2 Hz, 2H), 3.12 (q, J = 8.3 Hz, 1H), 2.63 (s, 1H), 2.03 (d, J = |
| | | 9.5 Hz, 1H), 1.87 (d, J = 5.9 Hz, |
| | | 1H), 1.83-1.64 (m, 5H), 1.18 (s, |
| | | 3H). 19F NMR (377 MHz, |
| | | DMSO-D6) δ −169.11. MS (ESI) |
| | | calcd. for C₂₄H₂₉FN₄O₇ 504.20 |
| | | m/z, found [M + H]⁺ 505.30 m/z. |
| 126 | 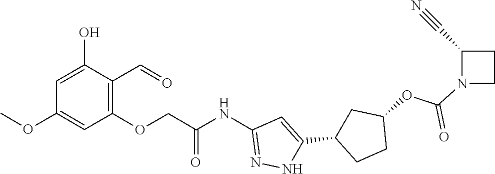 [SMILES: COc1cc(O)c(C=O)c(OCC(=O)Nc2cc([C@H]3CC[C@@H](OC(=O)N4CC[C@H]4C#N)C3)[nH]n2)c1] | ¹H NMR (400 MHz, DMSO-D6) δ 12.32 (s, 1H), 10.56 (s, 1H), 10.15 (s, 1H), 6.32 (s, 1H), 6.14-6.15 (m, 1H), 6.06-6.07 (m, 1H), 5.00-5.05 (m, 2H), 4.81 (s, 2H), 3.86-3.90 (m, 2H), 3.81 (s, 3H), 3.08-3.12 (m, 1H), 2.44-2.46 (m, 1H), 2.41- 2.43 (m, 2H), 2.02-2.08 (m, 1H), 1.89-1.93 (m, 1H), 1.69-1.78 (m, 3H). MS (ESI) calcd. for C₂₃H₂₅N₅O₇, 483.18 m/z, found |
| | | [M + H]⁺ 484.25 m/z. |
| 127 | 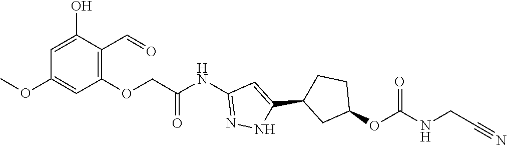 [SMILES: COc1cc(O)c(C=O)c(OCC(=O)Nc2cc([C@H]3CC[C@@H](OC(=O)NCC#N)C3)[nH]n2)c1] | ¹H NMR (300 MHz, DMSO-D6) δ 12.32 (s, 1H), 10.56 (s, 1H), 10.15 (s, 1H), 7.87 (s, 1H), 6.31 (s, 1H), 6.07-6.14 (m, 2H), 5.06-5.07 (m, 1H), 4.81 (s, 2H), 4.04-4.05 (m, 2H), 3.81 (s, 3H), 3.04-3.09 (m, 1H), 2.51-2.55 (m, 1H), 1.90-2.03 (m, 2H), 1.61-1.76 (m, 3H). MS (ESI) calcd. for C₂₁H₂₃N₅O₇ 457.16 |
| | | m/z, found [M + H]⁺ 458.25 m/z. |
| 129 | 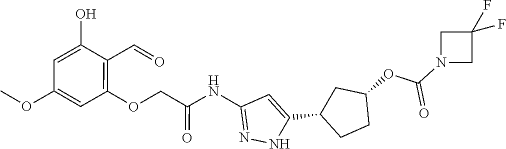 [SMILES: COc1cc(O)c(C=O)c(OCC(=O)Nc2cc([C@H]3CC[C@@H](OC(=O)N4CC(F)(F)C4)C3)[nH]n2)c1] | ¹H NMR (400 MHz, DMSO-D6) δ 12.32 (s, 1H), 10.56 (s, 1H), 10.15 (s, 1H), 6.33 (s, 1H), 6.14 (d, J = 2.1 Hz, 1H), 6.07 (d, J = 2.1 Hz, 1H), 5.10-5.01 (m, 1H), 4.81 (s, 2H), 4.37-4.25 (m, 4H), 3.81 (s, 3H), 3.18-3.04 (m, 1H), 2.48-2.36 (m, 1H), 2.07-1.95 (m, 1H), 1.95- 1.84 (m, 1H), 1.84-1.74 (m, 1H), |
| | | 1.74-1.65 (m, 3H). MS (ESI) mass |
| | | calcd. for C₂₂H₂₄F₂N₄O₇ 494.16 |
| | | m/z, found [M + H]⁺ 495.15 m/z. |
| 132 | 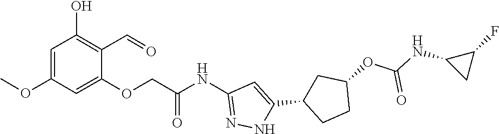 [SMILES: COc1cc(O)c(C=O)c(OCC(=O)Nc2cc([C@H]3CC[C@@H](OC(=O)N[C@H]4C[C@H]4F)C3)[nH]n2)c1] | ¹H NMR (400 MHz, DMSO-D6) δ 12.33 (s, 2H), 10.56 (s, 1H), 10.14 (s, 1H), 7.39 (s, 1H), 6.31 (s, 1H), 6.14 (d, J = 2.2 Hz, 1H), 6.06 (s, 1H), 5.02 (s, 1H), 4.80 (s, 2H), 4.69 (s, 1H), 3.80 (s, 3H), 3.05 (t, J = 8.9 Hz, 1H), 2.46 (s, 2H), 2.01 (d, J = 9.7 Hz, 2H), 1.90 (s, 2H), |
| | | 1.70-1.58 (m, 1H), 0.97-0.91 (m, |
| | | 2H). MS (ESI) calcd. for |
| | | C₂₂H₂₅FN₄O₇, 476.17 m/z, found |
| | | [M + H]⁺ 477.25 m/z. |
| 133 | 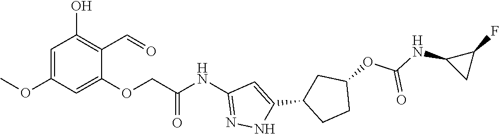 [SMILES: COc1cc(O)c(C=O)c(OCC(=O)Nc2cc([C@H]3CC[C@@H](OC(=O)N[C@@H]4C[C@@H]4F)C3)[nH]n2)c1] | ¹H NMR (400 MHz, DMSO-D6) δ 12.18-12.30 (m, 2H), 10.56 (s, 1H), 10.15 (s, 1H), 7.39 (s, 1H), 6.32 (s, 1H), 6.06-6.14 (m, 2H), 5.02 (s, 1H), 4.80 (s, 2H), 4.53- 4.69 (m, 1H), 3.80 (s, 3H), 3.45 (s, 1H), 3.06 (t, J = 9.7 Hz, 1H), 2.46 (s, 1H), 1.88-2.01 (m, 2H), 1.60- |
| | | 1.80 (m, 3H), 0.70-1.10 (m, 2H). |
| | | MS (ESI) calcd. for C₂₂H₂₅FN₄O₇, |
| | | 476.17 m/z, found [M + H]⁺ |
| | | 477.15 m/z. |
| 134 | 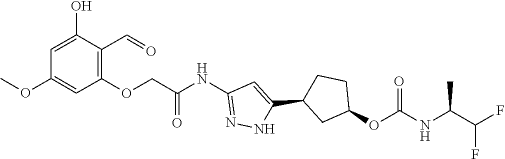 [SMILES: COc1cc(O)c(C=O)c(OCC(=O)Nc2cc([C@H]3CC[C@@H](OC(=O)N[C@@H](C)C(F)F)C3)[nH]n2)c1] | ¹H NMR (300 MHz, DMSO-D6) 12.32 (s, 1H), 12.18 (br s, 1H), 10.56 (s, 1H), 10.15 (s, 1H), 7.45 (d, t = 8.1 Hz, 1H), 6.32 (br s, 1H), 6.14-6.15 (m, 1H), 6.07-6.08 (m, 1H), 5.87-5.88 (m, 1H), 5.03 (br s, 1H), 4.81 (s, 2H), 3.81 (s, 4H), 3.05-3.34 (m, 1H), 2.50-2.51 (m, 1H), 2.00-2.03 (m, 1H), 1.97-1.99 (m, 1H), 1.67-1.87 (m, 2H), 1.57- |
| | | 1.65 (m, 1H), 1.07 (d, t = 6.9 Hz, |
| | | 3H). 19F NMR (282 MHz, |
| | | DMSO-D6) −127.4. MS (ESI) |
| | | calcd. For C₂₂H₂₆F₂N₄O₇, 496.18 |
| | | m/z, found [M + H]+ 497.15 m/z |
| 135 | 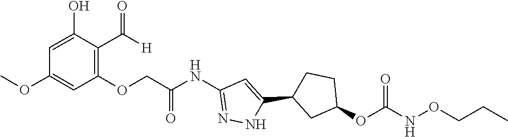 [SMILES: CCCONC(=O)O[C@@H]1CC[C@H](c2cc(NC(=O)COc3cc(OC)cc(O)c3C=O)n[nH]2)C1] | ¹H NMR (300 MHz, DMSO-D6) δ 12.32 (s, 1H), 10.54 (s, 1H), 10.15 (s, 2H), 6.31 (s, 1H), 6.07-6.14 (m, 2H), 5.05-5.07 (m, 1H), 4.81 (s, 2H), 3.81 (s, 3H), 3.62-3.66 (m, 2H), 3.02-3.13 (m, 1H), 2.44-2.47 (m, 1H), 1.88-2.06 (m, 2H), 1.56- 1.76 (m, 3H), 1.44-1.54 (m, 2H), 0.83-0.88 (m, 3H). MS (ESI) |
| | | calcd. for C₂₂H₂₈N₄O₈ 476.19 m/z, |
| | | found [M + H]⁺ 477.25 m/z. |
| 136 | 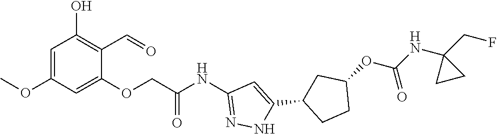 [SMILES: COc1cc(O)c(C=O)c(OCC(=O)Nc2cc([C@H]3CC[C@@H](OC(=O)NC4(CF)CC4)C3)[nH]n2)c1] | ¹H NMR (300 MHz, DMSO-D6) δ 12.18 (s, 1H), 10.55 (s, 1H), 10.13 (s, 1H), 7.60 (s, 1H), 6.29 (s, 1H), 6.16-6.08 (m, 2H), 4.99 (s, 1H), 4.79 (s, 2H), 4.38 (s, 1H), 4.22 (s, 1H), 3.79 (s, 3H), 3.03 (s, 1H), 2.48 (s, 1H), 2.08-1.83 (m, 2H), 1.68 (s, 3H), 0.74 (s, 4H). 19F |
| | | NMR (282 MHz, DMSO-D6) δ |
| | | (ppm): −214.905. MS (ESI) calcd. |
| | | for C₂₃H₂₇FN₄O₇, 490.19 m/z, |
| | | found [M + H]⁺, 491.25 m/z. |
| 137 | 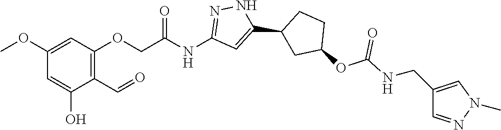 | ¹H NMR (400 MHz, DMSO-D6) δ 12.32 (s, 1H), 10.56 (s, 1H), 10.15 (s, 1H), 7.50 (s, 1H), 7.40 (t, J = 6.0 Hz, 1H), 7.26 (s, 1H), 6.30 (s, 1H), 6.14 (d, J = 2.1 Hz, 1H), 6.07 (d, J = 2.1 Hz, 1H), 5.04-4.96 (m, 1H), 4.81 (s, 2H), 3.97 (d, J = 5.9 Hz, 2H), 3.81 (s, 3H), 3.76 (s, 3H), |
| | | 3.10-2.99 (m, 1H), 2.47-2.42 (m, |
| | | 1H), 2.05-1.95 (m, 1H), 1.92-1.84 |
| | | (m, 1H), 1.77-1.64 (m, 2H), 1.61- |
| | | 1.57 (m, 1H) MS (ESI) calcd. for |
| | | C24H28N607 512.20 m/z, found |
| | | [M + H]⁺ 513.30 m/z. |
| 138 | 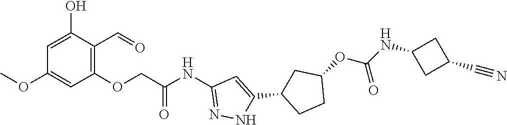 [SMILES: COc1cc(O)c(C=O)c(OCC(=O)Nc2cc([C@H]3CC[C@@H](OC(=O)N[C@H]4C[C@@H](C#N)C4)C3)[nH]n2)c1] | ¹H NMR (400 MHz, DMSO-D6) δ: 12.33 (s, 1H), 12.18 (br s, 1H), 10.56 (s, 1H), 10.15 (s, 1H), 7.55 (d, J = 8.0 Hz, 1H), 6.31 (s, 1H), 6.07-6.15 (m, 2H), 4.99-5.03 (m, 1H), 4.81 (s, 2H), 3.96-3.99 (m, 1H), 3.81 (s, 3H), 2.98-3.06 (m, 2H), 2.53-2.58 (m, 2H), 2.44-2.48 |
| | | (m, 1H), 2.08-2.26 (m, 2H), 1.99- |
| | | 2.01 (m, 1H), 1.85-1.89 (m, 1H), |
| | | 1.67-1.72 (m, 2H), 1.58-1.65 (m, |
| | | 1H). MS (ESI) calcd. for |
| | | C₂₄H₂₇N₅O₇ 497.19 m/z, found |
| | | [M + H]⁺ 498.25 m/z. |
| 139 | 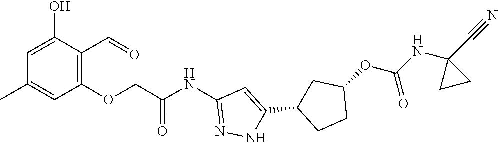 [SMILES: Cc1cc(O)c(C=O)c(OCC(=O)Nc2cc([C@H]3CC[C@@H](OC(=O)NC4(C#N)CC4)C3)[nH]n2)c1] | ¹H NMR (400 MHz, DMSO-D6) δ 12.20 (s, 1H), 11.72 (s, 1H), 10.53 (s, 1H), 10.30 (s, 1H), 8.25 (s, 1H), 6.39 (d, J = 9.9 Hz, 2H), 6.31 (s, 1H), 5.06 (s, 1H), 4.79 (s, 2H), 3.07 (s, 1H), 2.27 (s, 3H), 2.02 (s, 1H), 1.91 (s, 1H), 1.74 (s, 1H), 1.66 (s, 3H), 1.42 (d, J = 2.9 Hz, 2H), 1.18-1.11 (m, 2H). MS (ESI) |
| | | calcd. for C₂₃H₂₅N₅O₆, 467.18 m/z, |
| | | found [M + H]⁺ 468.25 m/z. |
| 141 | 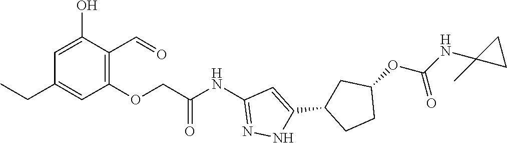 [SMILES: CCc1cc(O)c(C=O)c(OCC(=O)Nc2cc([C@H]3CC[C@@H](OC(=O)NC4(C)CC4)C3)[nH]n2)c1] | ¹H NMR (400 MHz, DMSO-D6) δ 12.16 (s, 1H), 11.77 (s, 1H), 10.51 (s, 1H), 10.31 (s, 1H), 7.36 (s, 1H), 6.42 (d, J = 3.2 Hz, 2H), 6.30 (s, 1H), 4.98 (s, 1H), 4.81 (s, 2H), 3.08-2.99 (m, 1H), 2.55 (q, J = 7.5 Hz, 2H), 2.45 (q, J = 7.5, 7.1 Hz, 1H), 1.99 (d, J = 9.1 Hz, 1H), 1.92- 1.82 (m, 1H), 1.76-1.62 (m, 2H), |
| | | 1.60-1.55 (m, 1H), 1.22 (s, 3H), |
| | | 1.15 (t, J = 7.6 Hz, 3H), 0.58 (s, |
| | | 2H), 0.46 (q, J = 4.4 Hz, 2H). MS |
| | | (ESI) calcd. for C₂₄H₃₀N₄O₆ 470.22 |
| | | m/z, found [M + H]⁺ 471.23 m/z. |
| 142 | 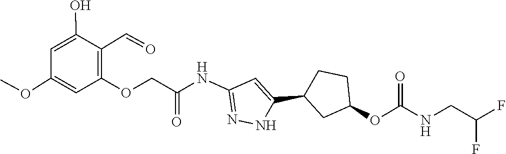 [SMILES: COc1cc(O)c(C=O)c(OCC(=O)Nc2cc([C@H]3CC[C@@H](OC(=O)NCC(F)F)C3)[nH]n2)c1] | ¹H NMR (400 MHz, DMSO-D6) δ 12.33 (s, 1H), 10.56 (s, 1H), 10.15 (s, 1H), 7.52-7.55 (m, 1H), 6.31 (s, 1H), 5.83-6.15 (m, 3H), 5.02-5.04 (m, 1H), 4.81 (s, 2H), 3.81 (s, 3H), 3.30-3.40 (m, 2H), 3.04-3.10 (m, 1H), 2.45-2.47 (m, 1H), 1.98-2.04 (m, 1H), 1.87-1.95 (m, 1H), 1.56- 1.75 (m, 3H). 19F NMR (376 MHz, DMSO-D6) δ: −74.31, |
| | | −122.47. MS (ESI) calcd. for |
| | | C₂₁H₂₄F₂N₄O₇ 482.16 m/z, found |
| | | [M + H]⁺ 483.30 m/z. |
| 143 | 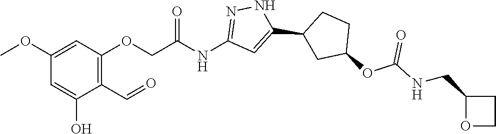 [SMILES: COc1cc(O)c(C=O)c(OCC(=O)Nc2cc([C@H]3CC[C@@H](OC(=O)NC[C@H]4CCO4)C3)[nH]n2)c1] | ¹H NMR (300 MHz, DMSO-D6) δ 12.33 (d, J = 3.3 Hz, 1H), 12.26 (s, 1H), 10.56 (s, 1H), 10.15 (s, 1H), 7.25 (t, J = 5.9 Hz, 1H), 6.31 (s, 1H), 6.15 (s, 1H), 6.04-6.18 (m, 1H), 5.01 (d, J = 6.8 Hz, 1H), 4.81 (d, J = 3.9 Hz, 2H), 4.59-4.71 (m, 1H), 4.40-4.52 (m, 1H), 4.30-4.39 (m, 1H), 3.82 (d, J = 2.6 Hz, 3H), |
| | | 3.12-3.30 (m, 2H), 2.97-3.10 (m, |
| | | 1H), 2.55-2.61 (m, 1H), 2.40-2.50 |
| | | (m, 1H), 2.21-2.35 (m, 1H), 1.95- |
| | | 2.10 (m, 1H), 1.80-1.90 (m, 1H), |
| | | 1.70-1.80 (m, 2H), 1.50-1.65 (m, |
| | | 1H). MS (ESI) calcd. for |
| | | C₂₃H₂₈N₄O₈, 488.19 m/z, found |
| | | [M + H]⁺ 489.05 m/z. |
| 144 | 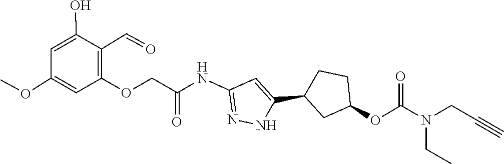 [SMILES: C#CCN(CC)C(=O)O[C@@H]1CC[C@H](c2cc(NC(=O)COc3cc(OC)cc(O)c3C=O)n[nH]2)C1] | ¹H NMR (400 MHz, DMSO-D6) δ 12.33 (s, 1H), 12.19 (s, 1H), 10.55 (s, 1H), 10.14 (s, 1H), 6.32 (s, 1H), 6.13-6.14 (m, 1H), 6.05-6.06 (m, 1H), 5.00-5.05 (m, 1H), 4.80 (s, 2H), 4.00-4.01 (m, 2H), 3.83 (s, 3H), 3.09-3.24 (m, 4H), 2.44-2.49 (m, 1H), 2.01-2.03 (m, 1H), 1.92- 1.99 (m, 1H), 1.88-1.90 (m, 2H), 1.65-1.71 (m, 1H), 1.02-1.04 (m, 3H). |
| 145 | 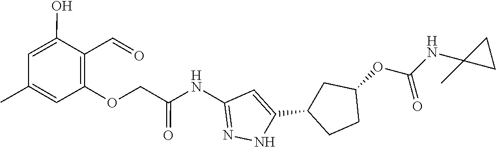 [SMILES: Cc1cc(O)c(C=O)c(OCC(=O)Nc2cc([C@H]3CC[C@@H](OC(=O)NC4(C)CC4)C3)[nH]n2)c1] | ¹H NMR (300 MHz, DMSO-D6) δ 12.17 (s, 1H), 10.52 (s, 1H), 10.31 (s, 1H), 7.35 (s, 1H), 6.39 (d, J = 7.6 Hz, 2H), 6.31 (s, 1H), 4.98 (s, 1H), 4.80 (s, 2H), 3.62 (s, 1H), 3.09-3.01 (m, 1H), 2.45 (m, 1H), 2.27 (s, 3H), 2.00 (d, J = 7.7 Hz, 1H), 1.93-1.78 (m, 1H), 1.56 (s, 3H), 1.23 (s, 3H), 0.58 (s, 2H), |
| | | 0.46 (s, 2H). MS (ESI) calcd. for |
| | | C₂₃H₂₈N₄O₆, 456.20 m/z, found |
| | | [M + H]⁺ 457.30 m/z. |
| 146 | 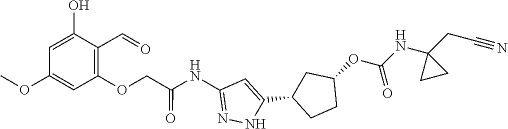 [SMILES: COc1cc(O)c(C=O)c(OCC(=O)Nc2cc([C@H]3CC[C@@H](OC(=O)NC4(CC#N)CC4)C3)[nH]n2)c1] | ¹H NMR (300 MHz, DMSO-D6) δ 12.33 (s, 1H), 12.18 (s, 1H), 10.56 (s, 1H), 10.15 (s, 1H), 7.83 (s, 1H), 6.32 (s, 1H), 6.15 (d, J = 2.1 Hz, 1H), 6.07 (d, J = 2.1 Hz, 1H), 5.02 (s, 1H), 4.81 (s, 2H), 3.81 (s, 3H), 3.20-3.05 (m, 1H), 2.80 (d, J = 3.6 Hz, 2H), 2.46 (s, 1H), 2.06-1.94 |
| | | (m, 1H), 1.90-1.80 (m, 1H), 1.72 |
| | | (d, J = 12.8 Hz, 2H), 1.68-1.55 (m, |
| | | 1H), 0.74 (s, 4H). MS (ESI) calcd. |
| | | for C₂₄H₂₇N₅O₇, 497.19 m/z, |
| | | found [M + H]⁺ 498.25 m/z. |
| 147 | 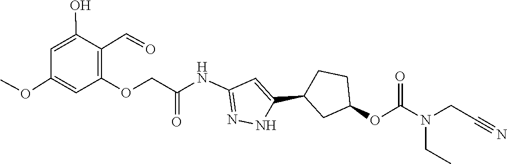 [SMILES: CCN(CC#N)C(=O)O[C@@H]1CC[C@H](c2cc(NC(=O)COc3cc(OC)cc(O)c3C=O)n[nH]2)C1] | ¹H NMR (400 MHz, DMSO-D6) δ 12.32 (s, 1H), 12.20 (s, 1H), 10.56 (s, 1H), 10.15 (s, 1H), 6.35 (s, 1H), 6.14 (d, J = 2.1 Hz, 1H), 6.06 (s, 1H), 5.10 (d, J = 4.6 Hz, 1H), 4.80 (s, 2H), 4.31 (s, 2H), 3.81 (s, 3H), 3.12 (s, 1H), 2.32 (s, 1H), 2.04- 1.89 (m, 3H), 1.83-1.67 (m, 4H), 1.06 (t, J = 7.0 Hz, 3H). MS (ESI) calcd. for C₂₃H₂₇N₅O₇, 485.19 |
| | | m/z, found [M + H]⁺ 485.90 m/z. |
| 148 | 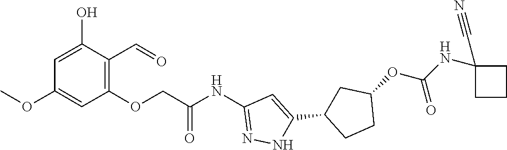 [SMILES: COc1cc(O)c(C=O)c(OCC(=O)Nc2cc([C@H]3CC[C@@H](OC(=O)NC4(C#N)CCC4)C3)[nH]n2)c1] | ¹H NMR (400 MHz, DMSO-D6) δ 12.32 (s, 1H), 10.56 (s, 1H), 10.15 (s, 1H), 8.27 (s, 1H), 6.32 (s, 1H), 6.14 (s, 1H), 6.07 (s, 1H), 5.06 (s, 1H), 4.81 (s, 2H), 3.81 (s, 3H), 3.18-2.90 (m, 1H), 2.57-2.52 (m, 3H), 2.35-2.23 (m, 2H), 2.08-1.88 (m, 4H), 1.83-1.68 (m, 2H), 1.69- 1.54 (m, 1H). MS (ESI) mass |
| | | calcd. for C₂₄H₂₇N₅O₇ 497.19 |
| | | m/z, found, [M + H]⁺ 498.25 m/z. |
| 149 | 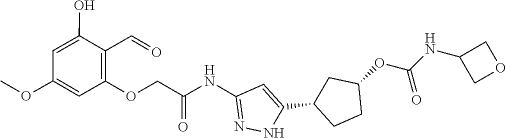 [SMILES: COc1cc(O)c(C=O)c(OCC(=O)Nc2cc([C@H]3CC[C@@H](OC(=O)NC4COC4)C3)[nH]n2)c1] | ¹H NMR (300 MHz, DMSO-D6) δ 12.33 (s, 1H), 12.19 (s, 1H), 10.57 (s, 1H), 10.15 (s, 1H), 7.91 (d, J = 5.4 Hz, 1H), 6.32 (s, 1H), 6.15 (d, J = 2.1 Hz, 1H), 6.07 (d, J = 2.1 Hz, 1H), 5.00 (d, J = 6.9 Hz, 1H), 4.81 (s, 2H), 4.67-4.60 (m, 3H), 4.42 (s, 2H), 3.81 (s, 3H), 3.12- 3.00 (m, 1H), 2.50-2.39 (m, 1H), |
| | | 2.12-1.98 (m, 1H), 1.95-1.90 (m, |
| | | 1H), 1.80-1.70 (m, 2H), 1.68-1.50 |
| | | (m, 1H). MS (ESI) calcd. for |
| | | C₂₂H₂₆N₄O₈, 474.18 m/z, found |
| | | [M + H]⁺ 475.25 m/z. |
| 150 | 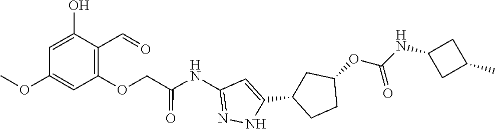 | ¹H NMR (300 MHz, DMSO-D6) δ 12.33 (s, 1H), 12.15-12.21 (m, 1H), 10.56 (s, 1H), 10.15 (s, 1H), 7.28 (d, J = 8.1 Hz, 1H), 6.31 (s, 1H), 6.15 (d, J = 2.1 Hz, 1H), 6.07 (d, J = 2.1 Hz, 1H), 4.98 (t, J = 6.9 Hz, 1H), 4.81 (s, 2H), 3.81 (s, 3H), 3.65-3.79 (m, 1H), 2.99-3.13 (m, |
| | | 1H), 2.37-2.51 (m, 1H), 2.21-2.36 |
| | | (m, 2H), 1.97-2.09 (m, 1H), 1.85- |
| | | 1.97 (m, 1H), 1.78-1.90 (m, 1H), |
| | | 1.61-1.78 (m, 2H), 1.46-1.60 (m, |
| | | 1H) 1.37-1.55 (m, 2H), 0.98 (d, J = |
| | | 6.5 Hz, 3H). MS (ESI) calcd. for |
| | | C₂₄H₃₀N₄O₇ 486.21 m/z, found |
| | | [M + H]⁺ 487.35 m/z. |
| 151 | 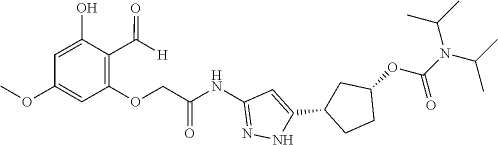 [SMILES: COc1cc(O)c(C=O)c(OCC(=O)Nc2cc([C@H]3CC[C@@H](OC(=O)N(C(C)C)C(C)C)C3)[nH]n2)c1] | ¹H NMR (300 MHz, DMSO-D6) δ 10.58 (br s, 1H), 10.23 (s, 1H), 7.53-8.63 (m, 2H), 6.79 (s, 1H), 5.98 (d, J = 1.8 Hz, 1H), 5.80 (d, J = 2.1 Hz, 1H), 5.14-5.21 (m, 1H), 4.68 (s, 2H), 3.84-4.07 (m, 1H), 3.74 (s, 3H), 3.56-3.70 (m, 1H), 3.09-3.24 (m, 1H), 2.51-2.63 (m, 1H), 2.11-2.21 (m, 1H), 1.92-2.00 |
| | | (m, 2H), 1.80-1.91 (m, 2H), 1.07- |
| | | 1.16 (m, 12H). MS (ESI) calcd. for |
| | | C₂₅H₃₄N₄O₇ 502.24 m/z, found |
| | | [M + H]⁺ 503.30 m/z. |
| 152 | 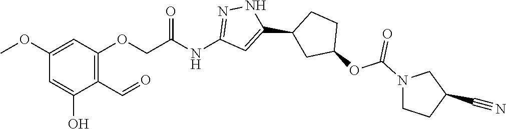 [SMILES: COc1cc(O)c(C=O)c(OCC(=O)Nc2cc([C@H]3CC[C@@H](OC(=O)N4CC[C@H](C#N)C4)C3)[nH]n2)c1] | ¹H NMR (400 MHz, DMSO-D6) δ 12.48-12.05 (m, 2H), 10.56 (s, 1H), 10.15 (s, 1H), 6.39-6.27 (m, 1H), 6.14 (d, J = 2.1 Hz, 1H), 6.06 (d, J = 2.1 Hz, 1H), 5.10-4.95 (m, 1H), 4.80 (s, 2H), 3.81 (s, 3H), 3.61-3.53 (m, 1H), 3.47-3.35 (m, 3H), 3.32-3.26 (m, 1H), 3.17-3.04 |
| | | (m, 1H), 2.47-2.36 (m, 1H), 2.25- |
| | | 2.12 (m, 1H), 2.12-1.97 (m, 2H), |
| | | 1.95-1.83 (m, 1H), 1.83-1.62 (m, |
| | | 3H), MS (ESI) calcd. for |
| | | C₂₄H₂₇N₅O₇ 497.19 m/z, found |
| | | [M + H]⁺ 498.15 m/z. |
| 153 | 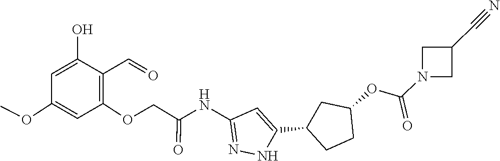 [SMILES: COc1cc(O)c(C=O)c(OCC(=O)Nc2cc([C@H]3CC[C@@H](OC(=O)N4CC(C#N)C4)C3)[nH]n2)c1] | ¹H NMR (400 MHz, DMSO-D6) δ 12.32 (s, 1H), 10.56 (s, 1H), 10.15 (d, J = 1.7 Hz, 1H), 6.32 (s, 1H), 6.15 (d, J = 2.0 Hz, 1H), 6.07 (d, J = 2.5 Hz, 1H), 5.03-4.97 (m, 1H), 4.81 (s, 2H), 4.18-4.09 (m, 2H), 4.07-3.77 (m, 3H), 3.78-3.69 (m, 1H), 3.16-3.03 (m, 1H), 2.47-2.34 (m, 1H), 2.05-1.98 (m, 1H), 1.92- 1.82 (m, 1H), 1.79-1.62 (m, 3H). |
| | | MS (ESI) mass calcd. for |
| | | C₂₃H₂₅N₅O₇ 483.18 m/z, found |
| | | [M + H]⁺ 484.30 m/z. |
| 154 | 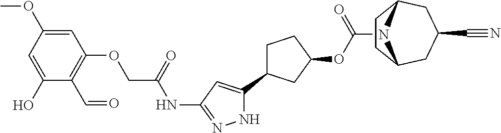 [SMILES: COc1cc(O)c(C=O)c(OCC(=O)Nc2cc([C@H]3CC[C@@H](OC(=O)N4[C@@H]5CC[C@H]4C[C@H](C#N)C5)C3)[nH]n2)c1] | ¹H NMR (400 MHz, DMSO-D6) δ 12.42-12.04 (m, 2H), 10.55 (s, 1H), 10.15 (s, 1H), 6.33 (s, 1H), 6.11 (dd, J = 30.5, 2.1 Hz, 2H), 5.08 (s, 1H), 4.80 (s, 2H), 4.18- 4.00 (m, 2H), 3.81 (s, 3H), 3.29- 3.19 (m, 1H), 3.18-3.08 (m, 1H), 2.45-2.35 (m, 1H), 2.10-1.97 (m, |
| | | 1H), 1.97-1.48 (m, 12H), MS (ESI) |
| | | calcd. for C₂₇H₃₁N₅O₇ 537.22 m/z, |
| | | found [M + H]⁺ 538.15 m/z. |
| 155 | 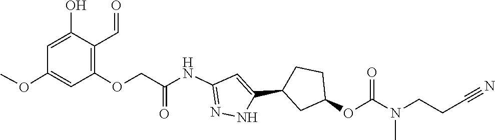 [SMILES: COc1cc(O)c(C=O)c(OCC(=O)Nc2cc([C@H]3CC[C@@H](OC(=O)N(C)CCC#N)C3)[nH]n2)c1] | ¹H NMR (300 MHz, DMSO-D6) δ 12.34-12.19 (m, 2H), 10.56 (s, 1H), 10.15 (s, 1H), 6.33 (s, 1H), 6.14 (d, J = 2.1 Hz, 1H), 6.06 (d, J = 2.1 Hz, 1H), 5.08 (s, 1H), 4.80 (s, 2H), 3.81 (s, 3H), 3.44 (d, J = 7.0 Hz, 2H), 3.11 (d, J = 8.2 Hz, 1H), 2.84 (s, 3H), 2.69 (t, J = 7.6 Hz, 2H), 2.42 (s, 1H), 2.03 (d, J = |
| | | 7.7 Hz, 1H), 1.77 (s, 4H). MS |
| | | (ESI) calcd. for C₂₃H₂₇N₅O₇, |
| | | 485.19 m/z, found [M + H]⁺ |
| | | 486.30 m/z. |
| 156 | 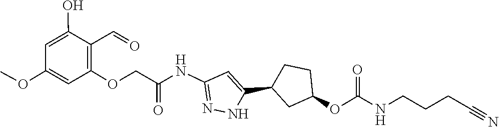 [SMILES: COc1cc(O)c(C=O)c(OCC(=O)Nc2cc([C@H]3CC[C@@H](OC(=O)NCCCC#N)C3)[nH]n2)c1] | ¹H NMR (300 MHz, DMSO-D6) δ 12.33 (s, 1H), 12.18 (s, 1H), 10.55 (s, 1H), 10.16 (s, 1H), 7.19 (t, J = 5.7 Hz, 1H), 6.32 (s, 1H), 6.15 (d, J = 2.1 Hz, 1H), 6.07 (d, J = 2.1 Hz, 1H), 5.01 (s, 1H), 4.81 (s, 2H), 3.81 (s, 3H), 3.04 (q, J = 6.5 Hz, 3H), 2.46 (d, J = 7.1 Hz, 2H), 2.11- |
| | | 1.83 (m, 1H), 1.78-1.52 (m, 1H), |
| | | 1.51 (s, 6H). MS (ESI) calcd for |
| | | C₂₃H₂₇N₅O₇: 485.19 m/z, found |
| | | [M + H]⁺ 486.05 m/z. |
| 157 | 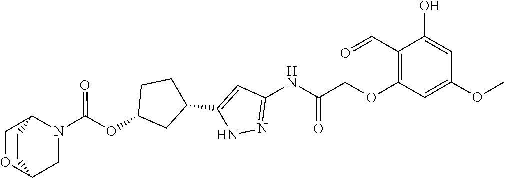 [SMILES: COc1cc(O)c(C=O)c(OCC(=O)Nc2cc([C@H]3CC[C@@H](OC(=O)N4C[C@@H]5CC[C@H]4CO5)C3)[nH]n2)c1] | ¹H NMR (400 MHz, DMSO-D6) δ (ppm): 12.33 (br s, 1H), 12.18 (br s, 1H), 10.55 (s, 1H), 10.14 (s, 1H), 6.31-6.35 (m, 1H), 6.13-6.14 (m, 1H), 6.06-6.07 (m, 1H), 5.05- 5.06 (m, 1H), 4.80 (s, 2H), 3.72- 3.92 (m, 7H), 3.49-3.58 (m, 1H), 3.35-3.36 (m, 1H), 3.09-3.15 (m, 1H), 2.36-2.46 (m, 1H), 1.60-2.03 (m, 9H). MS(ESI) calcd for C₂₅H₃₀N₄O₈ 514.21 m/z, found |
| | | [M + H]⁺ 515.15 m/z. |
| 158 | 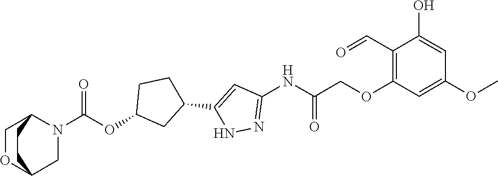 [SMILES: COc1cc(O)c(C=O)c(OCC(=O)Nc2cc([C@H]3CC[C@@H](OC(=O)N4C[C@H]5CC[C@@H]4CO5)C3)[nH]n2)c1] | ¹H NMR (400 MHz, DMSO-D6) δ (ppm): 12.33 (br s, 1H), 12.18 (br s, 1H), 10.55 (s, 1H), 10.14 (s, 1H), 6.31-6.33 (m, 1H), 6.13-6.14 (m, 1H), 6.06-6.07 (m, 1H), 5.04- 5.07 (m, 1H), 4.80 (s, 2H), 3.69- 3.91 (m, 7H), 3.49-3.57 (m, 1H), 3.30-3.31 (m, 1H), 3.11-3.15 (m, 1H), 2.36-2.40 (m, 1H), 2.00-2.03 (m, 1H), 1.85-1.91 (m, 2H), 1.70- 1.80 (m, 5H), 1.62-1.65 (m, 1H). |
| | | MS(ESI) calcd for C₂₅H₃₀N₄O₈ |
| | | 514.21 m/z, found [M + H]⁺ |
| | | 515.15 m/z. |
| 159 | 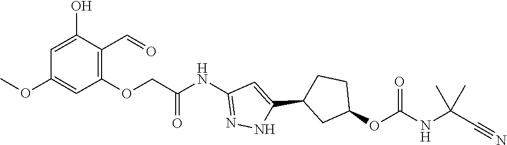 [SMILES: COc1cc(O)c(C=O)c(OCC(=O)Nc2cc([C@H]3CC[C@@H](OC(=O)NC(C)(C)C#N)C3)[nH]n2)c1] | ¹H NMR (400 MHz, DMSO-D6) δ 12.32 (s, 1H), 10.57 (s, 1H), 10.15 (s, 1H), 7.89 (s, 1H), 6.32 (s, 1H), 6.14-6.15 (m, 1H), 6.07-6.08 (m, 1H), 5.07-5.08 (m, 1H), 4.82 (s, 2H), 3.81(s, 3H), 3.03-3.12 (m, 1H), 2.50-2.56 (m, 1H), 1.98-2.02 (m, 2H), 1.60-1.97 (m, 5H), 1.51- 1.58 (m, 6H). MS (ESI) calcd. for |
| | | C₂₃H₂₇N₅O₇, 485.19 m/z, found |
| | | 486.10 [M + H]⁺ m/z. |
| 160 | 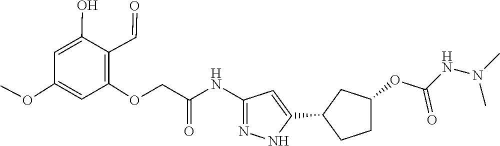 | ¹H NMR (400 MHz, DMSO-D6) δ 12.32 (s, 1H), 12.18 (s, 1H), 10.55 (s, 1H), 10.14 (s, 1H), 8.16 (br s, 1H), 6.32 (s, 1H), 6.13 (d, J = 2.0 Hz, 1H), 6.05 (d, J = 1.6 Hz, 1H), 5.00 (br s, 1H), 4.80 (s, 2H), 3.80 (s, 3H), 3.06 (br s, 1H), 2.40-2.42 (m, 7H), 2.00-2.03 (m, 1H), 1.85- 1.87 (m, 1H), 1.59-1.69 (m, 3H). |
| | | MS (ESI) calcd. for C₂₁H₂₇N₅O₇, |
| | | 461.19 m/z, found [M + H]⁺ |
| | | 462.30 m/z. |
| 161 | 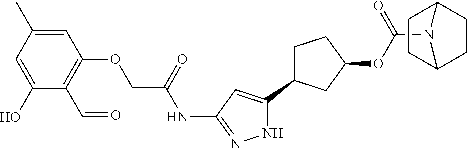 [SMILES: Cc1cc(O)c(C=O)c(OCC(=O)Nc2cc([C@H]3CC[C@@H](OC(=O)N4C5CCC4CC5)C3)[nH]n2)c1] | ¹H NMR (400 MHz, DMSO-D6) δ 12.17 (s, 1H), 11.77 (s, 1H), 10.51 (s, 1H), 10.30 (s, 1H), 6.42-6.33 (m, 3H), 4.99 (s, 1H), 4.79 (s, 2H), 4.07 (p, J = 2.5 Hz, 2H), 3.10 (t, J = 8.1 Hz, 1H), 2.40 (dt, J = 14.7, 7.1 Hz, 1H), 2.27 (s, 3H), 2.10- 1.96 (m, 1H), 1.94-1.82 (m, 1H), 1.80-1.68 (m, 2H), 1.65 (ddd, J = |
| | | 12.8, 7.7, 3.8 Hz, 1H), 1.61-1.55 |
| | | (m, 4H), 1.34 (d, J = 7.4 Hz, 4H). |
| | | MS (ESI) calcd. for C₂₅H₃₀N₄O₆ |
| | | 482.22 m/z, found [M + H]⁺ |
| | | 483.36 m/z. |
| 162 | 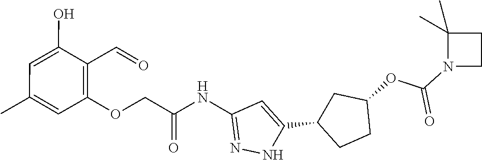 [SMILES: Cc1cc(O)c(C=O)c(OCC(=O)Nc2cc([C@H]3CC[C@@H](OC(=O)N4CCC4(C)C)C3)[nH]n2)c1] | ¹H NMR (500 MHz, DMSO-D6) δ 12.17 (d, J = 9.5 Hz, 1H), 11.77 (s, 1H), 10.50 (s, 1H), 10.31 (s, 1H), 6.40 (d, J = 9.4 Hz, 3H), 5.02 (dq, J = 6.6, 4.2, 3.1 Hz, 1H), 4.99-4.95 (m, 2H), 3.73 (h, J = 8.2 Hz, 1H), 3.65 (dd, J = 9.0, 6.1 Hz, 1H), 3.12-3.05 (m, 1H), 2.43 (m, 1H), 2.28 (s, 3H), 2.03 (t, J = 9.2 Hz, 1H), 1.93 (q, J = 7.8 Hz, 3H), 1.73 |
| | | (t, J = 10.2 Hz, 3H), 1.37 (d, J = |
| | | 4.0 Hz, 6H). MS (ESI) calcd. for |
| | | C₂₄H₃₀N₄O₆, 470.22 m/z, found |
| | | [M + H]⁺ 471.05 m/z. |
| 163 | 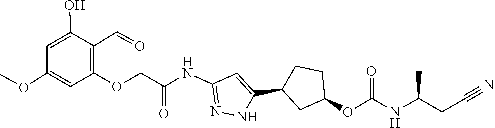 [SMILES: COc1cc(O)c(C=O)c(OCC(=O)Nc2cc([C@H]3CC[C@@H](OC(=O)N[C@@H](C)CC#N)C3)[nH]n2)c1] | ¹H NMR (500 MHz, DMSO-D6) δ 12.32 (s, 1H), 10.56 (s, 1H), 10.15 (d, J = 4.5 Hz, 1H), 7.39 (d, J = 7.7 Hz, 1H), 6.32 (s, 1H), 6.15 (d, J = 2.2 Hz, 1H), 6.08 (d, J = 2.2 Hz, 1H), 5.02 (s, 1H), 4.81 (d, J = 4.0 Hz, 2H), 3.81 (d, J = 3.9 Hz, 3H), 3.79 (m, 1H), 3.06 (s, 1H), 2.72- |
| | | 2.56 (m, 2H), 2.47 (s, 1H), 2.02 (d, |
| | | J = 9.8 Hz, 1H), 1.90 (s, 1H), 1.72 |
| | | (dd, J = 16.1, 10.4 Hz, 2H), 1.61 |
| | | (s, 1H), 1.14 (d, J = 6.7 Hz, 3H). |
| | | MS (ESI) calcd. for C₂₃H₂₇N₅O₇, |
| | | 485.19 m/z, found [M + H]⁺ |
| | | 486.25 m/z. |
| 164 | 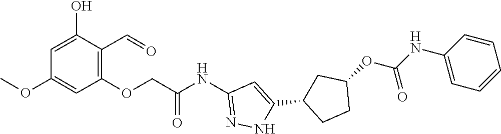 [SMILES: COc1cc(O)c(C=O)c(OCC(=O)Nc2cc([C@H]3CC[C@@H](OC(=O)Nc4ccccc4)C3)[nH]n2)c1] | ¹H NMR (400 MHz, DMSO-D6) δ 12.32 (s, 1H), 10.56 (s, 1H), 10.15 (s, 1H), 9.57 (s, 1H), 7.45 (d, J = 8.0 Hz, 2H), 7.25 (t, J = 7.8 Hz, 2H), 6.96 (t, J = 7.4 Hz, 1H), 6.34 (s, 1H), 6.11 (dd, J = 28.5, 2.1 Hz, 2H), 5.23-5.00 (m, 1H), 4.81 (s, 2H), 3.81 (s, 3H), 3.24-2.95 (m, |
| | | 1H), 2.80-2.55 (m, 1H), 2.10-2.03 |
| | | (m, 1H), 2.01-1.92 (m, 1H), 1.88- |
| | | 1.81 (m, 1H), 1.83-1.75 (m, 1H), |
| | | 1.76-1.64 (m, 2H). MS (ESI) mass |
| | | calcd. for C₂₅H₂₆N₄O₇ 494.18 m/z, |
| | | found [M + H]⁺ 495.25 m/z. |
| 165 | 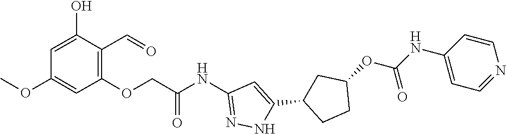 [SMILES: COc1cc(O)c(C=O)c(OCC(=O)Nc2cc([C@H]3CC[C@@H](OC(=O)Nc4ccncc4)C3)[nH]n2)c1] | ¹H NMR (300 MHz, DMSO-D6) 6.47 (s, 1H), 6.13 (s, 1H), 6.10- 5.98 (m, 1H), 5.20 (s, 1H), 5.05- 4.72 (m, 2H), 4.15 (d, J = 5.9 Hz, 2H), 3.89-3.79 (m, 3H), 3.06-2.94 (m, 5H), 2.60-2.49 (m, 2H), 2.45 (s, 3H), 2.24-2.10(m, 1H), 2.04- 1.84 (m, 4H), 1.75-1.64 (m, 2H). MS (ESI) calcd. for C₂₄H₂₅N₅O₇ |
| | | 495.18 m/z, found [M + H]⁺ |
| | | 496.25 m/z. |
| 166 | 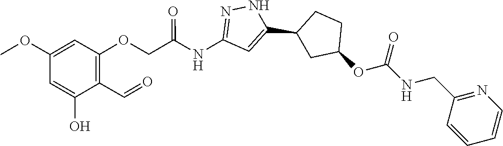 [SMILES: COc1cc(O)c(C=O)c(OCC(=O)Nc2cc([C@H]3CC[C@@H](OC(=O)NCc4ccccn4)C3)[nH]n2)c1] | ¹H NMR (400 MHz, DMSO-D6) δ 12.32 (s, 1H), 10.56 (s, 1H), 10.15 (s, 1H), 8.58 (d, J = 5.1 Hz, 1H), 7.97-8.06 (m, 1H), 7.78-7.87 (m, 1H), 7.45-7.54 (m, 2H), 6.33 (s, 1H), 6.14-6.17 (m, 1H), 6.07-6.09 (m, 1H), 5.02 (s, 1H), 4.82 (s, 2H), 4.34-4.41 (m, 2H), 3.81-3.87 (m, 3H), 3.08-3.14 (m, 1H), 2.03-2.12 |
| | | (m, 1H), 1.89-1.94 (m, 1H), 1.69- |
| | | 1.82 (m, 3H). MS (ESI) calcd. for |
| | | C₂₅H₂₇N₅O₇ 509.19 m/z, found |
| | | [M + H]⁺ 510.30 m/z. |
| 167 | 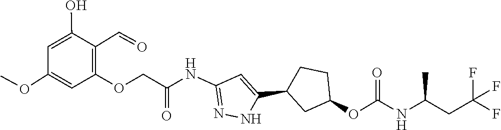 [SMILES: COc1cc(O)c(C=O)c(OCC(=O)Nc2cc([C@H]3CC[C@@H](OC(=O)N[C@@H](C)CC(F)(F)F)C3)[nH]n2)c1] | ¹H NMR (400 MHz, DMSO-D6) δ 12.33 (s, 1H), 10.56 (s, 1H), 10.15 (s, 1H), 7.20 (d, J = 8.4 Hz, 1H), 6.31 (s, 1H), 6.14-6.15 (m, 1H), 6.07-6.08 (m, 1H), 4.98-5.01 (m, 1H), 4.81 (s, 2H), 3.81-3.86 (m, 4H), 3.01-3.07 (m, 1H), 2.30-2.48 (m, 3H), 1.98-2.02 (m, 2H), 1.54- |
| | | 1.76 (m, 3H), 1.07-1.13 (m, 3H). |
| | | MS (ESI) calcd. For C₂₃H₂₇F₃N₄O₇, |
| | | 528.18 m/z, found [M + H]+ |
| | | 529.15 m/z. |
| 168 | 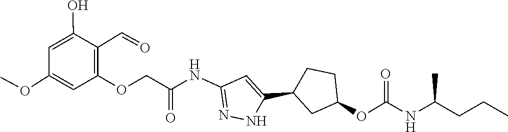 [SMILES: CCC[C@H](C)NC(=O)O[C@@H]1CC[C@H](c2cc(NC(=O)COc3cc(OC)cc(O)c3C=O)n[nH]2)C1] | ¹H NMR (400 MHz, DMSO-D6) δ 12.33 (s, 1H), 12.17 (s, 1H), 10.55 (s, 1H), 10.15 (s, 1H), 6.88 (d, J = 8.0 Hz, 1H), 6.32 (s, 1H), 6.15 (d, J = 2.0 Hz, 1H), 6.07 (d, J = 1.6 Hz, 1H), 4.97-4.99 (m, 1H), 4.81 (s, 2H), 3.81 (s, 3H), 3.43-3.48 (m, 1H), 3.02-3.07 (m, 1H), 2.43-2.46 |
| | | (m, 1H), 1.97-2.02 (m, 1H), 1.83- |
| | | 1.93 (m, 1H), 1.66-1.72 (m, 2H), |
| | | 1.53-1.60 (m, 1H), 1.24-1.34 (m, |
| | | 4H), 0.99-1.01 (m, 3H), 0.81-0.84 |
| | | (m, 3H). MS (ESI) calcd. for |
| | | C₂₄H₃₂N₄O₇, 488.23 m/z, found |
| | | [M + H]⁺ 489.20 m/z. |
| 169 | 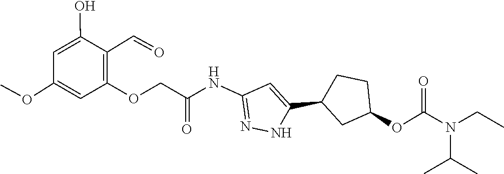 [SMILES: CCN(C(=O)O[C@@H]1CC[C@H](c2cc(NC(=O)COc3cc(OC)cc(O)c3C=O)n[nH]2)C1)C(C)C] | ¹H NMR (400 MHz, DMSO-D6) δ 12.32 (s, 1H), 10.54 (s, 1H), 10.15 (s, 1H), 6.31 (s, 1H), 6.13-6.14 (m, 1H), 6.06-6.07 (m, 1H), 5.04-5.05 (m, 1H), 4.80 (s, 2H), 3.99-4.15 (m, 1H), 3.80 (s, 3H), 3.06-3.12 (m, 3H), 2.38-2.46 (m, 1H), 1.90- 2.04 (m, 1H), 1.78-1.89 (m, 1H), 1.65-1.76 (m, 3H), 0.99-1.05 (m, 9H). MS (ESI) calcd. for C₂₄H₃₂N₄O₇, 488.23 m/z, found |
| | | [M + H]⁺ 489.20 m/z. |
| 170 | 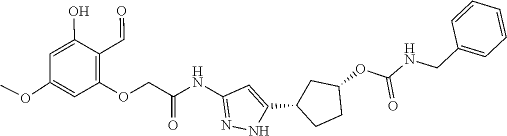 [SMILES: COc1cc(O)c(C=O)c(OCC(=O)Nc2cc([C@H]3CC[C@@H](OC(=O)NCc4ccccc4)C3)[nH]n2)c1] | ¹H NMR (400 MHz, DMSO-D6) δ: 12.33 (m, 1H), 10.57 (s, 1H), 10.16 (s, 1H), 7.64-7.67 (m, 1H), 7.20-7.33 (m, 5H), 6.33 (s, 1H), 6.15 (s, 1H), 6.10 (s, 1H), 5.01- 5.03 (m, 1H), 4.82 (s, 2H), 4.17(d, J = 6.0 Hz, 2 H), 3.81 (s, 3H), 3.02-3.08 (m, 1H), 2.50-2.52 (m, 1H), 1.89-2.03 (m, 2H), 1.58-1.78 |
| | | (m, 3H). MS (ESI) calcd. for |
| | | C₂₆H₂₈N₄O₇, 508.20 m/z, found |
| | | 509.15 [M + H]⁺ m/z. |
| 171 | 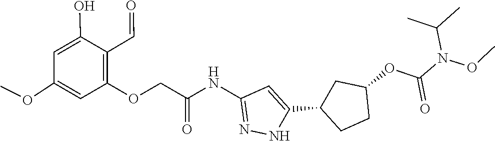 | ¹H NMR (300 MHz, DMSO-D6) δ (ppm): 12.32 (br s, 1H), 10.56 (s, 1H), 10.15 (s, 1H), 6.33 (s, 1H), 6.14 (d, J = 2.1 Hz, 1H), 6.07 (d, J = 2.1 Hz, 1H), 5.06-5.14 (m, 1H), 4.81 (s, 2H), 3.99-4.10 (m, 1H), 3.80 (s, 3H), 3.59 (s, 3H), 3.06- 3.19 (m, 1H), 2.41-2.48 (m, 1H), 1.98-2.09 (m, 1H), 1.89-1.97 (m, |
| | | 1H), 1.66-1.84 (m, 3H), 1.02-1.11 |
| | | (m, 6H). |
| 173 | 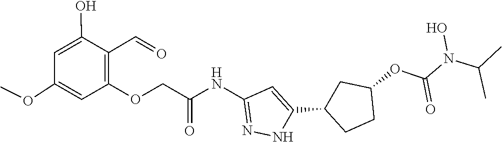 [SMILES: COc1cc(O)c(C=O)c(OCC(=O)Nc2cc([C@H]3CC[C@@H](OC(=O)N(O)C(C)C)C3)[nH]n2)c1] | ¹H NMR (400 MHz, DMSO-D6) δ 12.32 (s, 1H), 12.17 (s, 1H), 10.56 (s, 1H), 10.14 (s, 1H), 8.94 (s, 1H), 6.33 (s, 1H), 6.13-6.14 (m, 1H), 6.06-6.07 (m, 1H), 5.03-5.05 (m, 1H), 4.80 (s, 2H), 4.09-4.12 (m, 1H), 3.80 (s, 3H), 3.07-3.11 (m, 1H), 2.44-2.46 (m, 1H), 2.01-2.03 (m, 1H), 1.89-1.92 (m, 1H), 1.65- |
| | | 1.77 (m, 3H), 1.00-1.02 (m, 6H). |
| | | MS (ESI) calcd. for C₂₂H₂₈N₄O₈, |
| | | 476.19 m/z, found [M + H]⁺ |
| | | 477.25 m/z. |
| 175 | 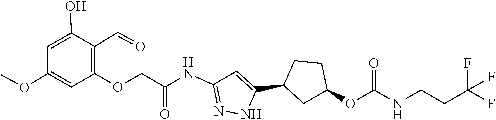 [SMILES: COc1cc(O)c(C=O)c(OCC(=O)Nc2cc([C@H]3CC[C@@H](OC(=O)NCCC(F)(F)F)C3)[nH]n2)c1] | ¹H NMR (400 MHz, DMSO-D6) δ 12.33 (s, 1H), 10.56 (s, 1H), 10.15 (s, 1H), 7.27-7.31 (m, 1H), 6.31 (s, 1H), 6.15 (s, 1H), 6.07 (s, 1H), 4.96-5.02 (m, 1H), 4.81 (s, 2H), 3.81 (s, 3H), 3.18-3.23 (m, 2H), 3.01-3.17 (m, 1H), 2.32-2.48 (m, 3H), 1.60-2.08 (m, 5H). MS (ESI) |
| | | calcd. for C₂₂H₂₅F₃N₄O₇, 514.17 |
| | | m/z, found [M + H]⁺ 515.15 m/z. |
| 176 | 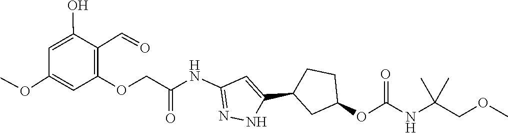 | ¹H NMR (300 MHz, DMSO-D6) δ 12.32 (s, 1H), 10.56 (s, 1H), 10.15 (s, 1H), 6.63 (br s, 1H), 6.31 (s, 1H), 6.14-6.15 (m, 1H), 6.06-6.07 (m, 1H), 4.91-4.97 (m, 1H), 4.81 (s, 2H), 3.81 (s, 3H), 3.27-3.29 (m, 2H), 3.23 (s, 3H), 2.97-3.09 (m, 1H), 2.41-2.46 (m, 1H), 1.96-2.01 |
| | | (m, 1H), 1.79-1.89 (m, 1H), 1.64- |
| | | 1.74 (m, 2H), 1.51-1.61 (m, 1H), |
| | | 1.14 (s, 6H). MS (ESI) calcd. for |
| | | C₂₄H₃₂N₄O₈ 504.22 m/z, found |
| | | [M + H]⁺ 505.20 m/z. |
| 177 | 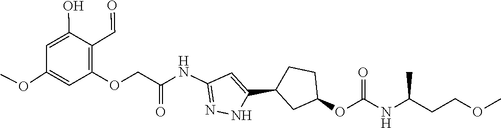 | ¹H NMR (400 MHz, DMSO-D6) δ: 12.18-12.34 (m, 2H), 10.56 (s, 1H), 10.15 (s, 1H), 6.91-6.93 (m, 1H), 6.32 (br s, 1H), 6.14 (br s, 1H), 6.07 (br s, 1H), 4.99 (br s, 1H), 4.81 (s, 2H), 3.81 (s, 3H), 3.51-3.59 (m, 1H), 3.23-3.29 (m, 2H), 3.18 (s, 3H), 3.01-3.09 (m, |
| | | 1H), 2.43-2.47 (m, 1H), 1.98-2.02 |
| | | (m, 1H), 1.83-1.93 (m, 1H), 1.68- |
| | | 1.73 (m, 2H), 1.51-1.66 (m, 3H), |
| | | 1.01-1.02 (m, 3H). MS (ESI) |
| | | calcd. for C₂₄H₃₂N₄O₈, 504.22 |
| | | m/z, found 505.20 [M + H]⁺. |
| 178 | 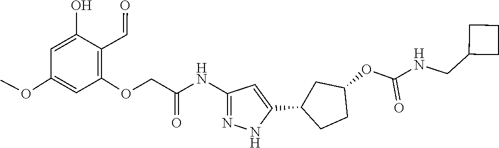 [SMILES: COc1cc(O)c(C=O)c(OCC(=O)Nc2cc([C@H]3CC[C@@H](OC(=O)NCC4CCC4)C3)[nH]n2)c1] | ¹H NMR (400 MHz, DMSO-D6) δ 12.31 (s, 1H), 12.17 (s, 1H), 10.56 (s, 1H), 10.14 (s, 1H), 7.06-7.08 (m, 1H), 6.31 (s, 1H), 6.13-6.14 (m, 1H), 6.06 (s, 1H), 4.99-5.00 (m, 1H), 4.80 (s, 2H), 3.80 (s, 3H), 2.95-3.10 (m, 3H), 2.44-2.46 (m, 1H), 2.33-2.36 (m, 1H), 1.86-2.01 (m, 4H), 1.63-1.79 (m, 4H), 1.58- |
| | | 1.61 (m, 3H). MS (ESI) calcd. for |
| | | C₂₄H₃₀N₄O₇, 486.21 m/z, found |
| | | [M + H]⁺ 487.30 m/z. |
| 179 | 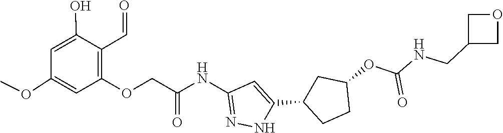 [SMILES: COc1cc(O)c(C=O)c(OCC(=O)Nc2cc([C@H]3CC[C@@H](OC(=O)NCC4COC4)C3)[nH]n2)c1] | ¹H NMR (400 MHz, DMSO-D6) δ 12.34 (s, 1H), 12.18 (s, 1H), 10.56 (s, 1H), 10.15 (s, 1H), 7.26 (t, J = 5.6 Hz, 1H), 6.31 (s, 1H), 6.07-6.14 (m, 2H), 4.97-5.02 (m, 1H), 4.81 (s, 2H), 4.57 (t, J = 7.2 Hz, 2H), 4.24 (t, J = 6.0 Hz, 2H), 3.81 (s, 3H), 3.23 (t, J = 6.4 Hz, 2H), 2.96-3.09 (m, 2H), 2.43-2.47 |
| | | (m, 1H), 1.97-2.03 (m, 1H), 1.84- |
| | | 1.94 (m, 1H), 1.55-1.75 (m, 3H). |
| | | MS (ESI) calcd. for C₂₃H₂₈N₄O₈, |
| | | 488.19 m/z, found [M + H]⁺ |
| | | 489.30 m/z. |
| 180 | 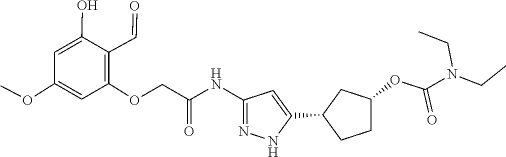 [SMILES: CCN(CC)C(=O)O[C@@H]1CC[C@H](c2cc(NC(=O)COc3cc(OC)cc(O)c3C=O)n[nH]2)C1] | ¹H NMR (400 MHz, DMSO-D6) δ (ppm): 12.33 (s, 1H), 10.58 (s, 1H), 10.15 (s, 1H), 6.32 (s, 1H), 6.14-6.15 (m, 1H), 6.06-6.07 (m, 1H), 5.02-5.04 (m, 1H), 4.81 (s, 2H), 3.81 (s, 3H), 3.09-3.15 (m, 5H), 2.40-2.46 (m, 1H), 2.02-2.04 (m, 1H), 1.87-1.90 (m, 1H), 1.65- 1.78 (m, 3H), 0.99 (m, 6H). MS(ESI) calcd for C₂₃H₃₀N₄O₇ |
| | | 474.21 m/z, found [M + H]⁺ |
| | | 475.20 m/z. |
| 181 | 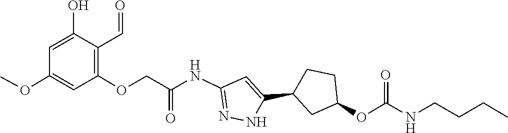 [SMILES: CCCCNC(=O)O[C@@H]1CC[C@H](c2cc(NC(=O)COc3cc(OC)cc(O)c3C=O)n[nH]2)C1] | ¹H NMR (400 MHz, DMSO-D6) δ 12.33 (s, 1H), 10.56 (s, 1H), 10.15 (s, 1H), 7.02-7.05 (m, 1H), 6.31 (s, 1H), 6.15 (s, 1H), 6.07 (s, 1H), 4.98-5.00 (m, 1H), 4.81 (s, 2), 3.81 (s, 3H), 3.01-3.08 (m, 1H), 2.89-2.98 (m, 2H), 2.41-2.46 (m, 1H), 1.95-2.05 (m, 1H), 1.84-1.91 (m, 1H), 1.66-1.74 (m, 2H), 1.54- |
| | | 1.62 (m, 1H), 1.18-1.40 (m, 4H), |
| | | 0.75-0.91 (m, 3H). MS (ESI) |
| | | calcd. for C₂₃H₃₀N₄O₇, 474.21 |
| | | m/z, found [M + H]⁺ 475.15 m/z. |
| 183 | 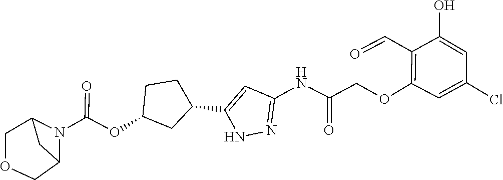 [SMILES: O=Cc1c(O)cc(Cl)cc1OCC(=O)Nc1cc([C@H]2CC[C@@H](OC(=O)N3C4COCC3C4)C2)[nH]n1] | ¹H NMR (400 MHz, DMSO-D6) δ 12.19 (s, 1H), 11.88 (s, 1H), 10.52 (s, 1H), 10.32 (s, 1H), 6.73-6.66 (m, 2H), 6.33 (s, 1H), 5.10 (s, 1H), 4.87 (s, 2H), 4.05 (d, J = 6.2 Hz, 4H), 3.65 (d, J = 10.2 Hz, 2H), 3.14-3.03 (m, 1H), 2.52-2.41 (m, 1H), 2.07-1.98 (m, 1H), 1.80-1.73 (m, 1H), 1.70-1.60 (m, 3H), 1.69 (s, 1H). MS (ESI) calcd. for C₂₃H₂₅CIN₄O₇, 504.14 m/z, |
| | | found [M + H]⁺ 505.05 m/z. |
| 184 | 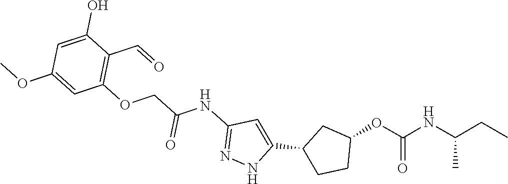 [SMILES: CC[C@H](C)NC(=O)O[C@@H]1CC[C@H](c2cc(NC(=O)COc3cc(OC)cc(O)c3C=O)n[nH]2)C1] | ¹H NMR (400 MHz, DMSO-D6) δ 12.33 (s, 1H), 10.56 (s, 1H), 10.15 (s, 1H), 6.88-6.90 (m, 1H), 6.31 (s, 1H), 6.14 (s, 1H), 6.07 (s, 1H), 4.98-4.99 (m, 1H), 4.80 (s, 2H), 3.81 (s, 3H), 3.33-3.41 (m, 1H), 3.02-3.06 (m, 1H), 2.42-2.47 (m, 1H), 1.23-2.03 (m, 7H), 0.93-1.08 (m, 3H), 0.86-0.92 (m, 3H). MS (ESI) calcd. for C₂₃H₃₀N₄O₇, 474.21 m/z, found [M + H]⁺ 475.10 m/z. |
| 185 | 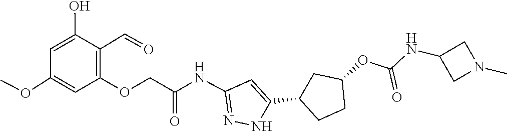 | ¹H NMR (400 MHz, DMSO-D6) δ 12.33 (s, 1H), 12.32 (br s, 1H), 10.59 (s, 1H), 10.15 (s, 1H), 9.70 (br s, 1H), 7.87-7.88 (m, 1H), 6.31 (s, 1H), 6.14-6.15 (m, 2H), 5.00- 5.02 (m, 1H), 4.82 (s, 2H), 4.25- 4.38 (m, 2H), 4.00-4.20 (m, 2H), 3.87-3.88 (m, 1H), 3.84 (s, 3H), |
| | | 3.06-3.10 (m, 1H), 2.76-2.82 (m, |
| | | 3H), 2.47-2.49 (m, 1H), 2.02-2.04 |
| | | (m, 1H), 1.86-1.91 (m, 1H), 1.63- |
| | | 1.79 (m, 3H). MS (ESI) calcd. for |
| | | C₂₃H₂₉N₅O₇, 487.21 m/z, found |
| | | [M + H]⁺ 488.30 m/z. |
| 186 | 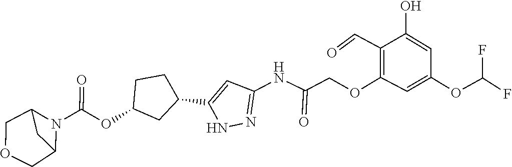 [SMILES: O=Cc1c(O)cc(OC(F)F)cc1OCC(=O)Nc1cc([C@H]2CC[C@@H](OC(=O)N3C4COCC3C4)C2)[nH]n1] | ¹H NMR (400 MHz, Methanol-d4) δ 7.04 (d, J = 4.4 Hz, 1H), 6.47- 6.43 (m, 1H), 6.37-6.29 (m, 2H), 5.99-5.91 (m, 1H), 5.29-5.13 (m, 2H), 4.74-4.67 (m, 2H), 4.19-4.08 (m, 4H), 3.74 (d, J = 9.8 Hz, 2H), 3.27-3.15 (m, 2H), 2.67-2.61 (m, 1H), 2.54-2.45 (m, 1H), 2.23-2.15 (m, 1H), 2.03-1.90 (m, 4H), 1.75- 1.70 (m, 1H). MS (ESI) mass |
| | | calcd. for C₂₄H₂₆F₂N₄O₈ 536.17 |
| | | m/z, found [M + H]⁺ 537.25 m/z. |
| 187 | 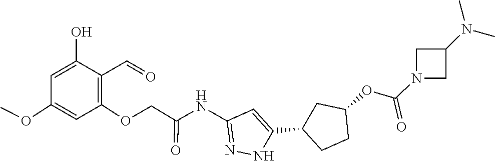 | ¹H NMR (400 MHz, DMSO-D6) δ 12.32 (br s, 1H), 10.62 (br s, 1H), 10.35 (br s, 1H), 10.14 (s, 1H), 6.35 (d, J = 2.7 Hz, 1H), 6.15 (d, J = 2.1 Hz, 1H), 6.06 (d, J = 1.8 Hz, 1H), 5.00-5.04 (m, 1H), 4.81 (s, 2H), 4.03-4.09 (m, 5H), 3.81 (s, 3H), 3.10-3.15 (m, 1H), 2.74 (s, 6H), 2.49-2.51 (m, 1H), 1.95-2.06 (m, 1H), 1.85-1.91 (m, 1H), 1.74- |
| | | 1.80 (m, 3H). MS (ESI) calcd. for |
| | | C₂₄H₃₁N₅O₇, 501.22 m/z, found |
| | | [M + H]⁺ 502.10 m/z. |
| 188 | 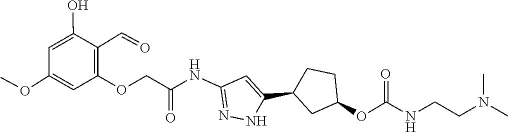 | ¹H NMR (400 MHz, DMSO-D6) δ 10.56 (s, 1H), 10.15 (s, 1H), 6.93- 6.96 (m, 1H), 6.31 (s, 1H), 6.15 (d, J = 2.0 Hz, 1H), 6.07 (d, J = 2.0 Hz, 1H), 4.96-5.01 (m, 1H), 4.81 (s, 2H), 3.81 (s, 3H), 3.02-3.07 (m, 3H), 2.43-2.48 (m, 1H), 2.26-2.30 (m, 2H), 2.09-2.14 (m, 6H), 1.97- |
| | | 2.02 (m, 1H), 1.86-1.93 (m, 1H), |
| | | 1.66-1.73 (m, 2H), 1.55-1.62 (m, |
| | | 1H). MS (ESI) calcd. for |
| | | C₂₃H₃₁N₅O₇, 489.22 m/z, found |
| | | [M + H]⁺ 490.30 m/z. |
| 189 | 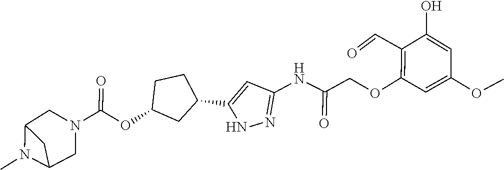 | ¹H NMR (300 MHz, DMSO-D6 + D2O) δ 10.15-10.16 (d, 1H), 6.34- 6.35 (m, 1H), 6.15-6.16 (m, 1H), 6.05-6.07 (m, 1H), 5.12-5.13 (m, 1H), 4.80-4.82 (m, 2H), 4.05-4.33 (m, 2H), 3.79-3.87 (m, 4H), 3.66- 3.75 (m, 3H), 3.15-3.20 (m, 2H), 2.96 (s, 1H), 2.66-2.81 (m, 1H), 2.41-2.54 (m, 2H), 2.06-2.10 (m, 1H), 1.90-1.93 (m, 5H). MS (ESI) calcd. for C₂₅H₃₁N₅O₇, 513.22 |
| | | m/z, found [M + H]⁺ 514.35 m/z. |
| 190 | 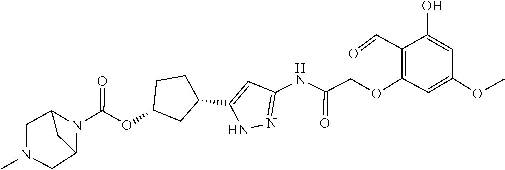 | ¹H NMR (300 MHz, DMSO-D6) 6.47 (s, 1H), 6.13 (s, 1H), 6.10- 5.98 (m, 1H), 5.20 (s, 1H), 5.05- 4.72 (m, 2H), 4.15 (d, J = 5.9 Hz, 2H), 3.89-3.79 (m, 3H), 3.06-2.94 (m, 5H), 2.60-2.49 (m, 2H), 2.45 (s, 3H), 2.24-2.10(m, 1H), 2.04- 1.84 (m, 4H), 1.75-1.64 (m, 2H). MS (ESI) calcd. for C₂₅H₃₁N₅O₇ 513.22 m/z m/z, found [M + H]⁺ 514.25 m/z. |
| 191 | 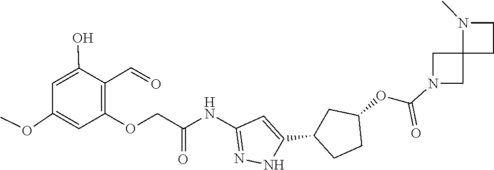 [SMILES: COc1cc(O)c(C=O)c(OCC(=O)Nc2cc([C@H]3CC[C@@H](OC(=O)N4CC5(CCN5C)C4)C3)[nH]n2)c1] | ¹H NMR (300 MHz, DMSO-D6) δ (ppm): 12.31 (s, 1H), 10.59 (s, 1H), 10.20 (br s, 1H), 10.15 (s, 1H), 6.33 (s, 1H), 6.15 (d, J = 1.8 Hz, 1H), 6.07 (d, J = 2.1 Hz, 1H), 4.97-5.06 (m, 1H), 4.82 (s, 2H), 4.26-4.49 (m, 2H), 3.93-4.14 (m, 4H), 3.81 (s, 3H), 3.03-3.19 (m, 1H), 2.84 (s, 3H), 2.56-2.68 (m, 2H), 2.36-2.47 (m, 1H), 1.99-2.09 (m, 1H), 1.60-1.95 (m, 4H). MS |
| | | (ESI) calcd. for C₂₅H₃₁N₅O₇, |
| | | 513.22 m/z, found [M + H]⁺ |
| | | 514.20 m/z. |
| 192 | 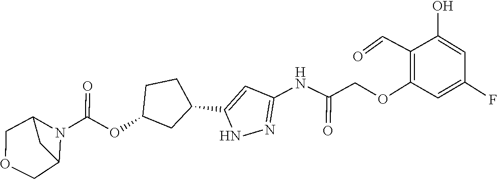 [SMILES: O=Cc1c(O)cc(F)cc1OCC(=O)Nc1cc([C@H]2CC[C@@H](OC(=O)N3C4COCC3C4)C2)[nH]n1] | ¹H NMR (300 MHz, DMSO-D6) δ 12.18 (s, 2H), 10.54 (s, 1H), 10.27 (s, 1H), 6.37-6.54 (m, 2H), 6.33 (s, 1H), 5.06-5.14 (m, 1H), 4.85 (s, 1H), 4.70 (s, 1H), 4.05 (d, J = 6.2 Hz, 4H), 3.64 (d, J = 10.3 Hz, 2H), 3.07-3.22 (m, 1H), 2.35-2.46 (m, 2H), 2.02 (t, J = 8.4 Hz, 1H), 1.89 (d, J = 6.4 Hz, 1H), 1.65-1.81 (m, 3H), 1.57 (d, J = 8.3 Hz, 1H). 19F NMR (282 MHz, DMSO-D6) δ |
| | | −96.17. MS (ESI) calcd. for |
| | | C₂₃H₂₅FN₄O₇, 488.17 m/z, found |
| | | [M + H]⁺ 489.05 m/z. |
| 193 | 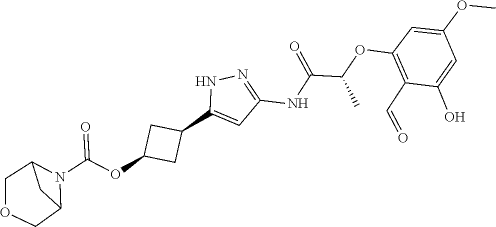 [SMILES: COc1cc(O)c(C=O)c(O[C@H](C)C(=O)Nc2cc([C@H]3C[C@@H](OC(=O)N4C5COCC4C5)C3)[nH]n2)c1] | ¹H NMR (400 MHz, DMSO-D6) δ (ppm): 12.27-12.34 (m, 2H), 10.69 (s, 1H), 10.15 (s, 1H), 6.35 (s, 1H), 6.13 (d, J = 2.0 Hz, 1H), 6.00 (d, J = 2.0 Hz, 1H), 5.01-5.03 (m, 1H), 4.86-4.90 (m, 1H), 4.02-4.09 (m, 4H), 3.78 (s, 3H), 3.66-3.71 (m, 2H), 3.03-3.12 (m, 1H), 2.68-2.69 (m, 2H), 2.51-2.55 (m, 1H), 2.08- 2.10 (m, 2H), 1.62-1.64 (m, 1H), 1.55 (d, J = 6.8 Hz, 3H). MS (ESI) calcd. for C₂₄H₂₈N₄O₈, 500.19 m/z, found [M + H]⁺ 501.10 m/z. |
| 194 | 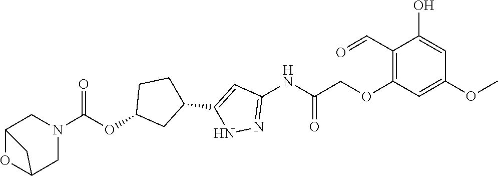 | ¹H NMR (400 MHz, DMSO-D6) δ (ppm): 12.16 (s, 1H), 10.30 (br s, 1H), 10.20 (s, 1H), 6.40 (d, J = 2.4 Hz, 1H), 6.36 (d, J = 2.4 Hz, 1H), 6.28 (br s, 1H), 5.06-5.12 (m, 1H), 4.02-4.08 (m, 4H), 3.79 (s, 3H), 3.62-3.68 (m, 2H), 3.04-3.18 (m, 3H), 2.54-2.62 (m, 2H), 2.35-2.47 (m, 2H), 1.84-2.06 (m, 2H), 1.57- 1.80 (m, 4H). MS (ESI) calcd. for C₂₅H₃₀N₄O₇, 498.21 m/z, found |
| | | [M + H]⁺ 499.25 m/z. |
| 195 | 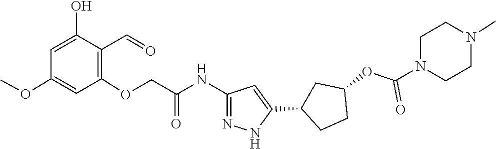 | ¹H NMR (400 MHz, DMSO-D6) δ 12.32 (s, 1H), 10.61 (s, 1H), 10.15 (s, 1H), 9.94 (s, 1H), 6.32 (s, 1H), 6.15-6.16 (d, 1H), 6.07-6.08 (d, 1H), 5.05-5.08 (m, 1H), 4.82 (s, 2H), 3.99-4.05 (m, 2H), 3.81 (s, 3H), 3.36 (s, 2H), 3.11-3.17 (m, 3H), 2.98-3.09 (m, 2H), 2.77 (s, 3H), 2.40-2.51 (m, 1H), 2.01-2.07 |
| | | (m, 1H), 1.86-1.95 (m, 1H), 1.70- |
| | | 1.84 (m, 3H). MS (ESI) calcd. for |
| | | C₂₄H₃₁N₅O₇, 501.22 m/z, found |
| | | [M + H]⁺ 502.35 m/z. |
| 196 | 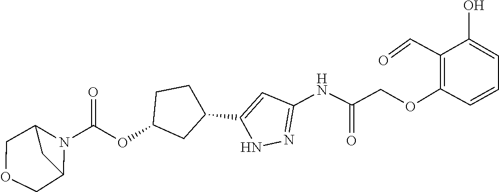 [SMILES: O=Cc1c(O)cccc1OCC(=O)Nc1cc([C@H]2CC[C@@H](OC(=O)N3C4COCC3C4)C2)[nH]n1] | ¹H NMR (400 MHz, DMSO-D6) δ 11.71 (s, 1H), 10.54 (s, 1H), 10.39 (s, 1H), 7.50 (t, J = 8.4 Hz, 1H), 6.51-6.57 (m, 2H), 6.32 (s, 1H), 5.09-5.10 (m, 1H), 4.82 (s, 2H), 3.90-4.05 (m, 4H), 3.63-3.65 (m, 2H), 3.09-3.14 (m, 1H), 2.43-2.48 (m, 2H), 1.88-2.04 (m, 2H), 1.66- 1.77 (m, 3H), 1.56-1.58 (m, 1H). MS (ESI) calcd. for C₂₃H₂₆N₄O₇, 470.18 m/z, found 471.15 [M + H]+. |
| 197 | 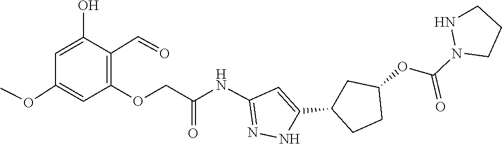 [SMILES: COc1cc(O)c(C=O)c(OCC(=O)Nc2cc([C@H]3CC[C@@H](OC(=O)N4CCCN4)C3)[nH]n2)c1] | ¹H NMR (400 MHz, DMSO-D6) δ 12.33 (br s, 1H), 12.21 (br s, 1H), 10.55 (s, 1H), 10.15 (s, 1H), 6.33 (s, 1H), 6.15 (s, 1H), 6.14 (s, 1H), 5.02-5.08 (m, 1H), 4.90-4.98 (m, 1H), 4.81 (s, 2H), 3.81 (s, 3H), 3.25-3.36 (m, 2H), 3.12-3.20 (m, 1H), 2.73-2.81 (m, 2H), 2.33-2.40 (m, 1H), 1.99-2.11 (m, 1H), 1.62- |
| | | 1.96 (m, 6H). MS (ESI) calcd. for |
| | | C₂₂H₂₇N₅O₇, 473.19 m/z, found |
| | | [M + H]⁺ 474.25 m/z. |
| 198 | 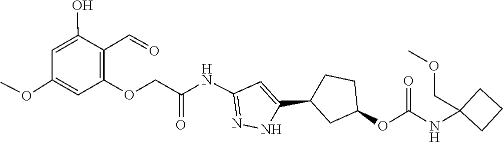 [SMILES: COCC1(NC(=O)O[C@@H]2CC[C@H](c3cc(NC(=O)COc4cc(OC)cc(O)c4C=O)n[nH]3)C2)CCC1] | ¹H NMR (400 MHz, DMSO-D6) δ (ppm): 12.32 (s, 1H), 10.57 (s, 1H), 10.15 (s, 1H), 7.12-7.13(m, 1H), 6.31 (s, 1H), 6.14-6.15 (m, 1H), 6.07-6.08 (m, 1H), 4.97-4.98 (m, 1H), 4.81 (s, 2H), 3.81 (s, 3H), 3.41 (s, 2H), 3.26 (s, 3H), 3.03- 3.07 (m, 1H), 2.43-2.49 (m, 1H), 1.87-2.10 (m, 6H), 1.58-1.75 (m, |
| | | 5H). MS(ESI) calcd for |
| | | C₂₅H₃₂N₄O₈ 516.22 m/z, found |
| | | [M + H]⁺ 517.30 m/z. |
| 199 | 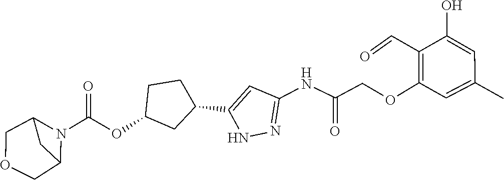 [SMILES: Cc1cc(O)c(C=O)c(OCC(=O)Nc2cc([C@H]3CC[C@@H](OC(=O)N4C5COCC4C5)C3)[nH]n2)c1] | ¹H NMR (300 MHz, DMSO-D6) δ 12.17 (s, 1H), 11.76 (s, 1H), 10.50 (s, 1H), 10.31 (s, 1H), 6.40 (d, J = 5.0 Hz, 2H), 6.33 (s, 1H), 5.09 (m, 1H), 4.80 (s, 2H), 4.05-3.81 (d, J = 6.2 Hz, 5H), 3.64 (d, J = 9.3 Hz, 2H), 3.12 (t, J = 8.1 Hz, 1H), 2.48- 2.32 (m, 1H), 2.28 (s, 3H), 2.03 (q, J = 8.4 Hz, 1H), 1.90 (t, J = 6.7 Hz, 1H), 1.81-1.58 (m, 3H), 1.55-1.51 (m, 1H). MS (ESI) calcd. for |
| | | C₂₄H₂₈N₄O₇, 484.20 m/z, found |
| | | [M + H]⁺ 485.25 m/z. |
| 200 | 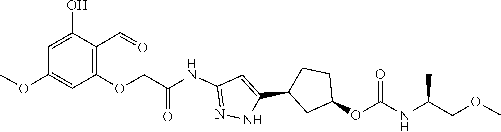 | ¹H NMR (400 MHz, DMSO-D6) δ 12.33 (s, 1H), 10.60-10.53 (m, 1H), 10.18-10.12 (m, 1H), 6.96 (d, J = 8.2 Hz, 1H), 6.34-6.29 (m, 1H), 6.15 (d, J = 2.2 Hz, 1H), 6.08 (d, J = 2.2 Hz, 1H), 5.04-4.97 (m, 1H), 4.85-4.78 (m, 2H), 3.84-3.80 (m, 3H), 3.72-3.62 (m, 1H), 3.26-3.19 |
| | | (m, 4H), 3.14-3.03 (m, 2H), 2.48- |
| | | 2.40 (m, 1H), 2.08-1.89 (m, 2H), |
| | | 1.82-1.57 (m, 3H), 1.00 (d, J = 6.8 |
| | | Hz, 3H). MS (ESI) mass calcd. for |
| | | C₂₃H₃₀N₄O₈ 490.21 m/z, found |
| | | [M + H]⁺ 491.20 m/z. |
| 201 | 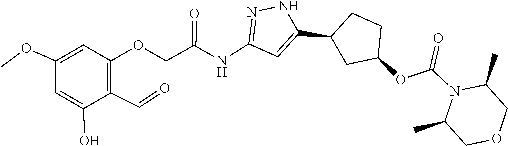 [SMILES: COc1cc(O)c(C=O)c(OCC(=O)Nc2cc([C@H]3CC[C@@H](OC(=O)N4[C@H](C)COC[C@@H]4C)C3)[nH]n2)c1] | ¹H NMR (400 MHz, DMSO-D6) δ 12.32 (s, 1H), 10.55 (s, 1H), 10.15 (s, 1H), 6.32 (s, 1H), 6.13-6.14 (m, 1H), 6.06-6.07 (m, 1H), 5.09-5.10 (m. 1H), 4.81 (s, 2H), 3.77-3.80 (m, 5H), 3.54-3.59 (m, 2H), 3.39- 3.43 (m, 2H), 3.09-3.14 (m, 1H), 2.30-2.45 (m, 1H), 2.01-2.07 (m, 1H), 1.87-1.91 (m, 1H), 1.72-1.78 |
| | | (m, 3H), 1.13-1.16 (m, 6H). MS |
| | | (ESI) calcd. for C₂₅H₃₂N₄O₈, |
| | | 516.22 m/z, found [M + H]⁺ |
| | | 517.30 m/z. |
| 202 | 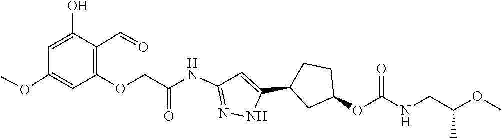 | ¹H NMR (300 MHz, DMSO-D6) δ 12.30 (br s, 1H), 12.15 (br s, 1H), 10.53 (br s, 1H), 10.13 (s, 1H), 7.03-7.07 (m, 1H), 6.30 (d, J = 3.0 Hz, 1H), 6.13 (d, J = 1.8 Hz, 1H), 6.05 (d, J = 2.1 Hz, 1H), 4.98-5.05 (m, 1H), 4.79 (s, 2H), 3.79 (s, 3H), 3.24-3.28 (m, 1H), 3.16-3.20 (m, 3H), 2.92-3.04 (m, 3H), 2.49-2.50 |
| | | (m, 1H), 1.87-2.01 (m, 2H), 1.57- |
| | | 1.72 (m, 3H), 0.95-1.03 (m, 3H). |
| | | MS (ESI) calcd. for C₂₃H₃₀N₄O₈, |
| | | 490.21 m/z, found [M + H]⁺ |
| | | 491.25 m/z. |
| 203 | 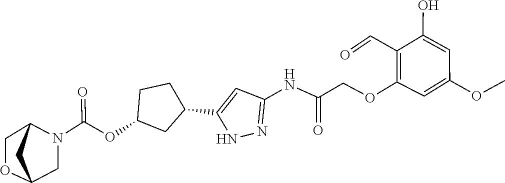 [SMILES: COc1cc(O)c(C=O)c(OCC(=O)Nc2cc([C@H]3CC[C@@H](OC(=O)N4C[C@H]5C[C@@H]4CO5)C3)[nH]n2)c1] | ¹H NMR (400 MHz, DMSO-D6) δ 12.32 (s, 1H), 10.55 (s, 1H), 10.15 (s, 1H), 6.29-6.33 (m, 1H), 6.14- 6.15 (m, 1H), 6.07-6.08 (m, 1H), 5.03-5.07 (m, 1H), 4.81 (s, 2H), 4.47-4.52 (m, 1H), 4.34-4.40 (m, 1H), 3.81 (s, 3H), 3.63-3.68 (m, 1H), 3.51-3.60 (m, 1H), 3.20-3.25 (m, 1H), 3.09-3.15 (m, 2H), 2.38- 2.41 (m, 1H), 2.00-2.03 (m, 1H), 1.87-1.90 (m, 1H), 1.75-1.80 (m, |
| | | 3H), 1.67-1.72 (m, 2H). MS (ESI) |
| | | calcd. for C₂₄H₂₈N₄O₈ 500.19 |
| | | m/z, found [M + H]⁺ 501.25 m/z. |
| 204 | 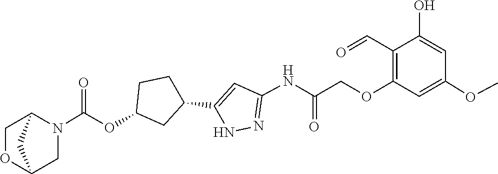 | ¹H NMR (400 MHz, DMSO-D6) δ 12.33 (s, 1H), 12.18 (s, 1H), 10.55 (s, 1H), 10.15 (s, 1H), 6.34 (s, 1H), 6.15 (s, 1H), 6.08 (s, 1H), 5.00- 5.01 (m, 1H), 4.81 (s, 2H), 4.33- 4.41 (m, 2H), 3.81 (s, 3H), 3.55- 3.65 (m, 2H), 3.08-3.33 (m, 3H), 2.36-2.43 (m, 1H), 1.90-2.02 (m, 2H), 1.59-1.85 (m, 5H). MS (ESI) calcd. for C₂₄H₂₈N₄O₈ 500.19 m/z, found [M + H]⁺ 501.10 m/z. |
| 205 | 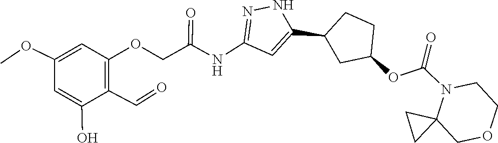 [SMILES: COc1cc(O)c(C=O)c(OCC(=O)Nc2cc([C@H]3CC[C@@H](OC(=O)N4CCOCC45CC5)C3)[nH]n2)c1] | ¹H NMR (300 MHz, DMSO-D6) δ 12.24-12.11 (m, 1H), 10.89-10.41 (m, 1H), 10.15 (s, 1H), 6.32 (s, 1H), 6.13 (d, J = 2.1 Hz, 1H), 6.05 (s, 1H), 5.07 (s, 1H), 4.80 (s, 2H), 4.62 (s, 1H), 3.81 (s, 3H), 3.66 (s, 1H), 3.55-3.45 (m, 2H), 3.43-3.39 (m, 3H), 3.12 (s, 1H), 2.03 (s, 1H), 1.99-1.82 (m, 1H), 1.76 (m, 4H), |
| | | 0.90 (s, 2H), 0.74 (s, 2H). MS |
| | | (ESI) calcd. for C₂₅H₃₀N₄O₈, |
| | | 514.21 m/z, found [M + H]⁺ |
| | | 515.30 m/z. |
| 206 | 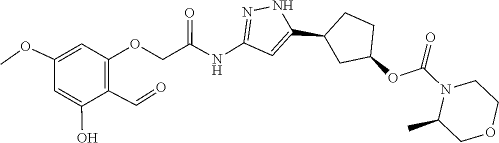 [SMILES: COc1cc(O)c(C=O)c(OCC(=O)Nc2cc([C@H]3CC[C@@H](OC(=O)N4CCOC[C@H]4C)C3)[nH]n2)c1] | ¹H NMR (400 MHz, DMSO-D6) δ (ppm): 12.32 (br s, 1H), 12.16 (br s, 1H), 10.55 (s, 1H), 10.15 (s, 1H), 6.32 (s, 1H), 6.14 (d, J = 1.6 Hz, 1H), 6.06 (d, J = 1.6 Hz, 1H), 5.03-5.09 (m, 1H), 4.80 (s, 2H), 3.85-3.93 (m, 1H), 3.80 (s, 3H), 3.70-3.77 (m, 1H), 3.47-3.56 (m, 2H), 3.40-3.45 (m, 1H), 3.23-3.28 |
| | | (m, 1H), 3.10-3.17 (m, 1H), 2.98- |
| | | 3.07 (m, 1H), 2.32-2.43 (m, 1H), |
| | | 1.98-2.07 (m, 1H), 1.84-1.95 (m, |
| | | 1H), 1.70-1.81 (m, 3H), 1.09 (d, |
| | | J = 6.8 Hz, 3H). MS (ESI) calcd. |
| | | for C₂₄H₃₀N₄O₈, 502.21 m/z, |
| | | found [M + H]⁺ 503.20 m/z. |
| 208 | 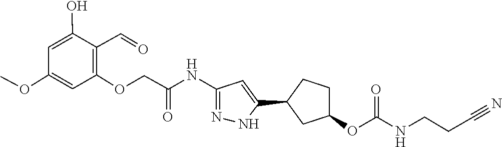 [SMILES: COc1cc(O)c(C=O)c(OCC(=O)Nc2cc([C@H]3CC[C@@H](OC(=O)NCCC#N)C3)[nH]n2)c1] | ¹H NMR (400 MHz, DMSO-D6) δ 12.34 (s, 1H), 10.55 (s, 1H), 10.15 (s, 1H), 7.41-7.49 (m, 1H), 6.30 (s, 1H), 6.18 (s, 1H), 6.06 (s, 1H), 4.98-5.07 (m, 1H), 4.83 (m, 2H), 3.82 (s, 3H), 3.18-3.25 (m, 2H), 3.00-3.15 (m, 1H), 2.59-2.67 (m, 2H), 2.43-2.46 (m, 1H), 1.93-2.03 (m, 1H), 1.83-1.92 (m, 1H), 1.66- |
| | | 1.77 (m, 2H), 1.54-1.63 (m, 1H). |
| | | MS(ESI) calcd. for C₂₂H₂₅N₅O₇, |
| | | 471.18 m/z, found [M + H]⁺ |
| | | 472.25. |
| 209 | 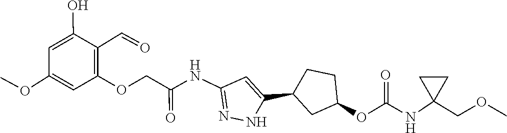 | ¹H NMR (400 MHz, DMSO-D6) δ 12.33 (s, 1H), 12.18 (s, 1H), 10.56 (s, 1H), 10.15 (s, 1H), 7.43 (s, 1H), 6.31 (br s, 1H), 6.14-6.15 (m, 1H), 6.07-6.08 (m, 1H), 4.94-5.00 (m, 1H), 4.81 (s, 2H), 3.81 (s, 3H), 3.31-3.35 (m, 2H), 3.17-3.23 (m, 3H), 2.98-3.08 (m, 1H), 2.45-2.48 (m, 1H), 1.94-2.04 (m, 1H), 1.62- |
| | | 1.90 (m, 3H), 1.66-1.71 (m, 1H), |
| | | 0.62 (s, 4H). MS (ESI) calcd. for |
| | | C₂₄H₃₀N₄O₈, 502.21 m/z, found |
| | | [M + H]⁺ 503.30 m/z. |
| 210 | 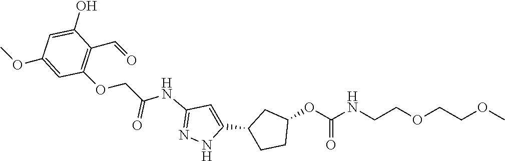 | ¹H NMR (400 MHz, DMSO-D6) δ 12.35 (br s, 1H), 10.62 (s, 1H), 10.16 (s, 1H), 7.01-7.07 (m, 1H), 6.33 (s, 1H), 6.13 (s, 1H), 6.07 (s, 1H), 4.97-5.02 (m, 1H), 4.82 (m, 2H), 3.81 (s, 3H), 3.34-3.53 (m, 6H), 3.22 (s, 3H), 3.04-3.12 (m, 3H), 2.43-2.49 (m, 1H), 1.94-2.03 (m, 1H), 1.83-1.92 (m, 1H), 1.66- 1.77 (m, 2H), 1.54-1.63 (m, 1H). |
| | | MS(ESI) calcd for C₂₄H₃₂N₄O₉, |
| | | 520.22 m/z, found [M + H]⁺ |
| | | 521.20. |
| 212 | 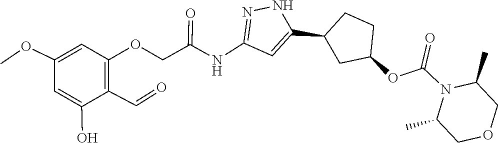 [SMILES: COc1cc(O)c(C=O)c(OCC(=O)Nc2cc([C@H]3CC[C@@H](OC(=O)N4[C@@H](C)COC[C@@H]4C)C3)[nH]n2)c1] | ¹H NMR (400 MHz, DMSO-D6) δ 12.14 (s, 2H), 10.53 (s, 1H), 10.14 (s, 1H), 6.32 (s, 1H), 6.13 (d, J = 2.1 Hz, 1H), 6.05 (d, J = 2.2 Hz, 1H), 5.06 (dq, J = 6.8, 3.6 Hz, 1H), 4.80 (s, 2H), 3.80 (s, 3H), 3.71 (s, 1H), 3.68 (d, J = 2.7 Hz, 3H), 3.34- 3.30 (m, 1H), 3.12 (s, 2H), 2.44 (td, J = 8.8, 8.4, 4.3 Hz, 1H), 2.08- |
| | | 1.96 (m, 1H), 1.91 (ddd, J = 12.8, |
| | | 9.5, 6.3 Hz, 1H), 1.80-1.65 (m, |
| | | 3H), 1.14 (d, J = 6.4 Hz, 6H). MS |
| | | (ESI) calcd. for C₂₅H₃₂N₄O₈ 516.22 |
| | | m/z, found [M + H]⁺ 517.20 m/z. |
| 213 | 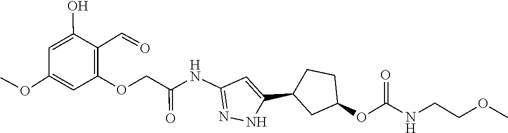 | ¹H NMR (400 MHz, DMSO-D6) δ 12.34 (s, 1H), 10.57 (s, 1H), 10.16 (s, 1H), 7.09-7.12 (m, 1H), 6.31 (s, 1H), 6.15 (m, 1H), 6.07-6.08 (m, 1H), 4.95-5.01 (m, 1H), 4.82 (s, 2H), 3.81 (s, 3H), 3.29-3.32 (m, 2H), 3.22 (s, 3H), 3.03-3.18 (m, 3H), 2.45-2.50 (m, 1H), 1.98-2.02 (m, 1H), 1.85-1.93 (m, 1H), 1.65- |
| | | 1.76 (m, 2H), 1.54-1.62 (m, 1H). |
| | | MS (ESI) calcd. for C₂₂H₂₈N₄O₈, |
| | | 476.19 m/z, found [M + H]⁺ |
| | | 477.15 m/z. |
| 214 | 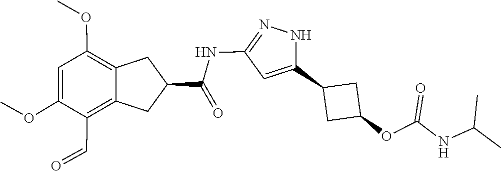 [SMILES: COc1cc(OC)c2c(c1C=O)C[C@H](C(=O)Nc1cc([C@H]3C[C@@H](OC(=O)NC(C)C)C3)[nH]n1)C2] | ¹H NMR (400 MHz, DMSO-D6) δ 12.22 (s, 1H), 10.41 (s, 1H), 10.28 (s, 1H), 7.07 (d, J = 7.8 Hz, 1H), 6.60 (s, 1H), 6.32 (s, 1H), 4.70- 4.90 (m, 1H), 3.92 (d, J = 4.3 Hz, 3H), 3.30-3.65 (m, 6H), 3.36 (d, J = 7.7 Hz, 1H), 2.75-3.20 (m, 3H), 2.70-2.59 (m, 2H), 2.05 (t, J = 9.9 Hz, 2H), 1.02 (d, J = 6.6 Hz, 6H). MS (ESI) calcd. for C₂₄H₃₀N₄O₆, 470.22 m/z, found [M + H]⁺ |
| | | 471.15 m/z. |
| 215 | 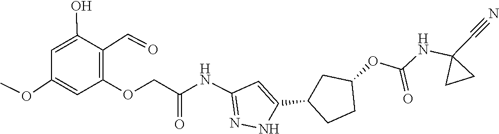 [SMILES: COc1cc(O)c(C=O)c(OCC(=O)Nc2cc([C@H]3CC[C@@H](OC(=O)NC4(C#N)CC4)C3)[nH]n2)c1] | ¹H NMR (400 MHz, DMSO-D6) δ 12.32 (s, 1H), 12.20 (s, 1H), 10.55 (s, 1H), 10.15 (s, 1H), 8.24 (s, 1H), 6.30 (s, 1H), 6.07 (d, 1H), 6.14 (d, 1H), 5.00-5.05 (m, 1H), 4.81 (s, 2H), 3.81 (s, 3H), 3.05-3.09 (m, 1H), 2.50-2.54 (m, 1H), 2.01-2.08 (m, 1H), 1.87-1.96 (m, 1H), 1.61- |
| | | 1.76 (m, 3H), 1.40-1.43 (m, 2H), |
| | | 1.12-1.16 (m, 2H). MS (ESI) |
| | | calcd. for C₂₃H₂₅N₅O₇, 483.18 m/z, |
| | | found [M + H]⁺ 484.15 m/z. |
| 216 | 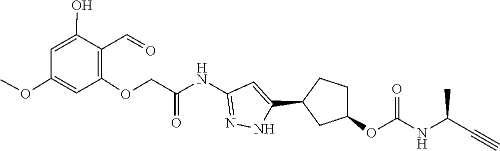 [SMILES: C#C[C@H](C)NC(=O)O[C@@H]1CC[C@H](c2cc(NC(=O)COc3cc(OC)cc(O)c3C=O)n[nH]2)C1] | ¹H NMR (400 MHz, DMSO-D6) δ (ppm): 12.32 (s, 1H), 12.17 (s, 1H), 10.54 (s, 1H), 10.15 (s, 1H), 7.59 (d, J = 8.2 Hz, 1H), 6.32 (s, 1H), 6.18-6.02 (m, 2H), 5.02 (s, 1H), 4.81 (s, 2H), 4.37-4.12 (m, 1H), 3.81 (s, 3H), 3.14-2.97 (m, 2H), 2.08-1.95 (m, 1H), 1.95-1.80 (m, 1H), 1.78-1.63 (m, 2H), 1.63- 1.51 (m, 1H), 1.34-1.18 (m, 4H). |
| | | MS (ESI) calcd. for C₂₃H₂₆N₄O₇: |
| | | 470.18 m/z, found [M + H]⁺ |
| | | 471.05 m/z. |
| 217 | 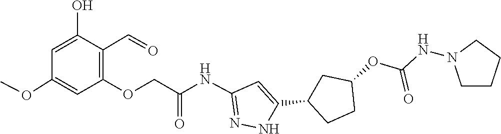 [SMILES: COc1cc(O)c(C=O)c(OCC(=O)Nc2cc([C@H]3CC[C@@H](OC(=O)NN4CCCC4)C3)[nH]n2)c1] | ¹H NMR (300 MHz, DMSO-D6) δ 12.32 (br s, 1H), 10.57 (br s, 1H), 10.15 (s, 1H), 6.32 (d, J = 3.9 Hz, 1H), 6.14 (d, J = 1.6 Hz, 1H), 6.07 (d, J = 2.1 Hz, 1H), 5.05-5.07 (m, 1H), 4.81 (s, 2H), 3.81 (s, 3H), 2.94-3.02 (m, 5H), 2.49-2.50 (m, 1H), 1.93-2.04 (m, 1H), 1.87-1.91 (m, 1H), 1.79-1.84 (m, 5H), 1.60- |
| | | 1.71 (m, 2H). MS (ESI) calcd. for |
| | | C₂₃H₂₉N₅O₇, 487.21 m/z, found |
| | | [M + H]⁺ 488.10 m/z. |
| 218 | 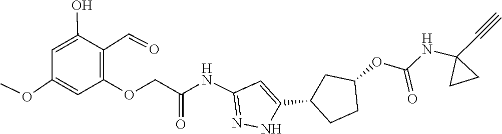 [SMILES: C#CC1(NC(=O)O[C@@H]2CC[C@H](c3cc(NC(=O)COc4cc(OC)cc(O)c4C=O)n[nH]3)C2)CC1] | ¹H NMR (400 MHz, DMSO-D6) δ 12.33 (s, 1H), 10.56 (s, 1H), 10.15 (s, 1H), 7.81 (s, 1H), 6.30-6.31 (m, 1H), 6.14-6.15 (m, 1H), 6.06-6.07 (m, 1H), 5.00-5.02 (m, 1H), 4.81 (s, 2H), 3.81 (m, 3H), 3.10-3.11 (m, 1H), 2.96 (s, 1H), 2.45-2.47 (m, 1H), 1.90-2.04 (m, 1H), 1.80- 1.89 (m, 1H), 1.60-1.70 (m, 2H), |
| | | 1.50-1.59 (m, 1H), 1.03-1.04 (m, |
| | | 2H), 0.95-1.02 (m, 2H). MS (ESI) |
| | | calcd. for C₂₄H₂₆N₄O₇, 482.18 |
| | | m/z, found [M + H]⁺ 483.10 m/z. |
| 219 | 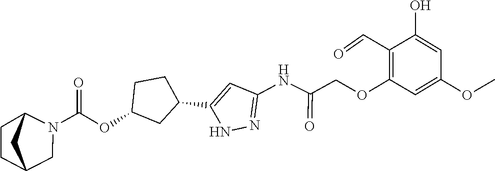 [SMILES: COc1cc(O)c(C=O)c(OCC(=O)Nc2cc([C@H]3CC[C@@H](OC(=O)N4C[C@@H]5CC[C@H]4C5)C3)[nH]n2)c1] | ¹H NMR (400 MHz, DMSO-D6) δ (ppm): 12.32 (s, 1H), 12.16 (s, 1H), 10.54 (s, 1H), 10.15 (s, 1H), 6.31-6.35 (m, 1H), 6.14-6.15 (m, 1H), 6.07 (s, 1H), 4.98-5.02 (m, 1H), 4.81 (s, 2H), 4.05-4.08 (m, 1H), 3.80 (s, 3H), 3.08-3.14 (m, 2H), 2.86-2.88 (m, 1H), 2.38-2.46 (m, 2H), 2.00-2.08 (m, 1H), 1.82- 1.92 (m, 1H), 1.50-1.77 (m, 7H), 1.28-1.30 (m, 2H). MS (ESI) |
| | | calcd. for C₂₅H₃₀N₄O₇ 498.21 |
| | | m/z, found [M + H]⁺ 499.20 m/z. |
| 220 | 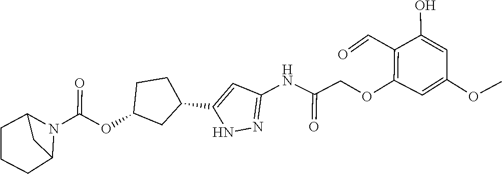 [SMILES: COc1cc(O)c(C=O)c(OCC(=O)Nc2cc([C@H]3CC[C@@H](OC(=O)N4C5CCCC4C5)C3)[nH]n2)c1] | ¹H NMR (400 MHz, DMSO-D6) δ 12.32 (br s, 1H), 10.56 (s, 1H), 10.15 (s, 1H), 6.32 (s, 1H), 6.14- 6.15 (m, 1H), 6.06-6.07 (m, 1H), 5.04-5.05 (m, 1H), 4.81 (s, 2H), 3.97-4.01 (m, 2H), 3.81 (s, 3H), 3.09-3.13 (m, 1H), 2.40-2.45 (m, 1H), 2.30-2.36 (m, 1H), 2.10-2.14 (m, 1H), 1.88-2.04 (m, 4H), 1.60- 1.77 (m, 5H), 1.47-1.52 (m, 1H), 1.38 (d, J = 8.8 Hz, 1H). MS (ESI) |
| | | calcd. for C₂₅H₃₀N₄O₇ 498.21 |
| | | m/z, found [M + H]⁺ 499.20 m/z. |
| 221 | 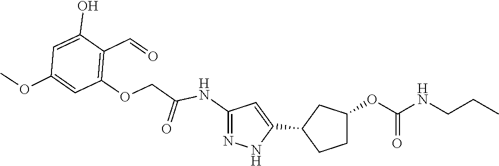 [SMILES: CCCNC(=O)O[C@@H]1CC[C@H](c2cc(NC(=O)COc3cc(OC)cc(O)c3C=O)n[nH]2)C1] | ¹H NMR (400 MHz, DMSO-D6) δ 12.33 (s, 1H), 10.56 (s, 1H), 10.15 (s, 1H), 7.05 (t, J = 5.6 Hz, 1H), 6.31 (s, 1H), 6.07-6.15 (m, 2H), 4.98-5.00 (m, 1H), 4.81 (s, 2H), 3.81 (s, 3H), 3.02-3.07 (m, 1H), 2.88-2.93 (m, 2H), 2.43-2.48 (m, 1H), 1.86-2.01 (m, 2H), 1.54-1.73 (m, 3H), 1.33-1.42 (m, 2H), 0.79- 0.83 (m, 3H). MS (ESI) calcd. for C₂₂H₂₈N₄O₇ 460.20 m/z, found |
| | | [M + H]⁺ 461.10 m/z. |
| 222 | 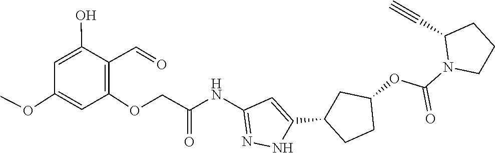 [SMILES: C#C[C@@H]1CCCN1C(=O)O[C@@H]1CC[C@H](c2cc(NC(=O)COc3cc(OC)cc(O)c3C=O)n[nH]2)C1] | ¹H NMR (300 MHz, DMSO-D6) δ (ppm): 12.44-12.04 (m, 2H), 10.54 (s, 1H), 10.14 (s, 1H), 6.32 (s, 1H), 6.17-6.00 (m, 2H), 5.08-4.99 (m, 1H), 4.80 (s, 2H), 4.46-4.38 (m, 1H), 3.80 (s, 3H), 3.07 (s, 3H), 2.30-2.24 (m, 1H), 2.12-1.93 (m, 2H), 1.92-1.57 (m, 8H). MS (ESI) calcd. for C₂₅H₂₈N₄O₇: 496.20 m/z, found: [M + H]⁺ 497.05. |
| 223 | 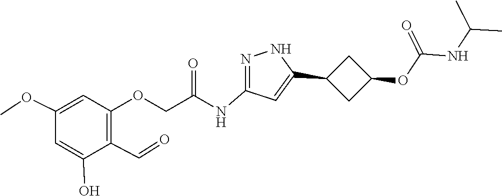 [SMILES: COc1cc(O)c(C=O)c(OCC(=O)Nc2cc([C@H]3C[C@@H](OC(=O)NC(C)C)C3)[nH]n2)c1] | ¹H NMR (500 MHz, DMSO-D6) δ 12.32 (s, 2H), 10.56 (s, 1H), 10.16 (s, 1H), 7.06 (d, J = 7.8 Hz, 1H), 6.35 (s, 1H), 6.15 (d, J = 2.1 Hz, 1H), 6.08 (d, J = 2.1 Hz, 1H), 4.88- 4.74 (m, 3H), 3.81 (s, 3H), 3.62- 3.52 (m, 1H), 3.12-3.01 (m, 1H), 2.71-2.62 (m, 2H), 2.10-2.01 (m, 2H), 1.04 (d, J = 6.5 Hz, 6H). MS (ESI) calcd. for C₂₁H₂₆N₄O₇ 446.18 m/z, found [M + H]⁺ 447.10 m/z. |
| 224 | 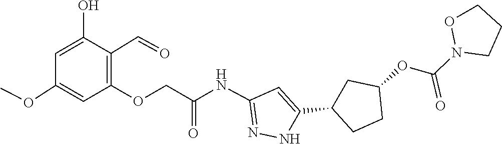 [SMILES: COc1cc(O)c(C=O)c(OCC(=O)Nc2cc([C@H]3CC[C@@H](OC(=O)N4CCCO4)C3)[nH]n2)c1] | ¹H NMR (400 MHz, DMSO-D6) δ 12.32 (s, 1H), 10.55 (s, 1H), 10.15 (s, 1H), 6.33 (s, 1H), 6.14-6.15 (m, 1H), 6.06-6.07 (m, 1H), 5.06-5.07 (m, 1H), 4.81 (s, 2H), 3.81 (s, 3H), 3.74-3.77 (m, 2H), 3.47-3.50 (m, 2H), 3.09-3.13 (m, 1H), 2.44-2.47 (m, 1H), 2.08-2.16 (m, 2H), 2.02- 2.04 (m, 1H), 1.90-1.91 (m, 1H), |
| | | 1.70-1.80 (m, 3H). MS (ESI) |
| | | calcd. for C₂₂H₂₆N₄O₈, 474.18 |
| | | m/z, found [M + H]⁺ 475.25 m/z. |
| 225 | 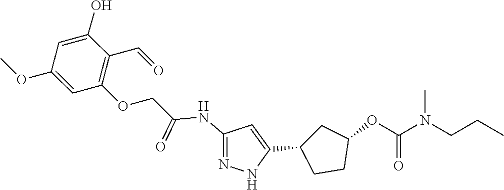 [SMILES: CCCN(C)C(=O)O[C@@H]1CC[C@H](c2cc(NC(=O)COc3cc(OC)cc(O)c3C=O)n[nH]2)C1] | ¹H NMR (400 MHz, DMSO-D6) δ (ppm): 12.32 (br s, 1H), 10.55 (s, 1H), 10.15 (s, 1H), 6.32 (s, 1H), 6.14 (d, J = 2.0 Hz, 1H), 6.06 (d, J = 2.0 Hz, 1H), 4.98-5.05 (m, 1H), 4.80 (s, 2H), 3.81 (s, 3H), 3.05-3.14 (m, 3H), 2.76 (s, 3H), 2.37-2.46 (m, 1H), 1.98-2.09 (m, 1H),1.82-1.93 (m, 1H), 1.72-1.79 (m, 2H), 1.63- 1.71 (m, 1H), 1.36-1.48 (m, 2H), 0.71-0.82 (m, 3H). MS (ESI) calcd. for C₂₃H₃₀N₄O₇, 474.21 m/z, found |
| | | [M + H]⁺ 475.15 m/z. |
| 226 | 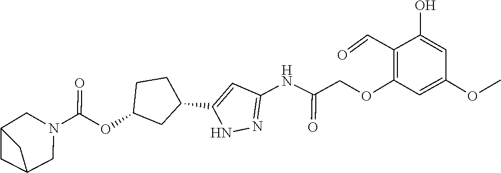 [SMILES: COc1cc(O)c(C=O)c(OCC(=O)Nc2cc([C@H]3CC[C@@H](OC(=O)N4CC5CC(C5)C4)C3)[nH]n2)c1] | ¹H NMR (400 MHz, DMSO-D6) δ (ppm): 12.32 (s, 1H), 10.54 (s, 1H), 10.14 (s, 1H), 6.34 (s, 1H), 6.15 (s, 1H), 6.07 (s, 1H), 5.06- 5.07 (m, 1H), 4.80 (s, 2H), 3.80 (s, 3H), 3.42-3.45 (m, 4H), 3.10-3.14 (m, 1H), 2.38-2.43 (m, 2H), 2.29- 2.30 (m, 1H), 2.03-2.06 (m, 3H), 1.87-1.88 (m, 1H), 1.72-1.80 (m, 3H), 1.22-1.26 (m, 2H). MS (ESI) calcd. for C₂₅H₃₀N₄O₇ 498.21 m/z, |
| | | found [M + H]⁺ 499.20 m/z. |
| 227 | 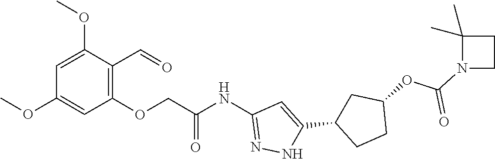 [SMILES: COc1cc(OC)c(C=O)c(OCC(=O)Nc2cc([C@H]3CC[C@@H](OC(=O)N4CCC4(C)C)C3)[nH]n2)c1] | ¹H NMR (300 MHz, DMSO-D6) δ 12.18 (br s, 1H), 10.32 (br s, 1H), 10.26(s, 1H), 6.34-6.37 (m, 2H), 6.30 (s, 1H), 5.02-5.03 (m, 1H), 4.75 (s, 2H), 3.88 (s, 6H), 3.72- 3.75 (m, 1H), 3.62-3.67 (m, 1H), 3.08-3.12 (m, 1H), 2.49-2.50 (m, 1H), 2.02-2.06 (m, 1H), 1.92-1.96 (m, 2H), 1.85-1.89 (m, 1H), 1.63- 1.75 (m, 3H), 1.31-1.37 (m, 6H). |
| | | MS (ESI) calcd. for C₂₅H₃₂N₄O₇, |
| | | 500.23 m/z, found [M + H]⁺ |
| | | 501.15 m/z. |
| 228 | 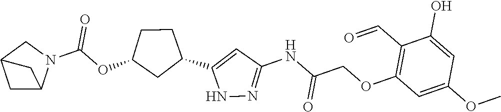 [SMILES: COc1cc(O)c(C=O)c(OCC(=O)Nc2cc([C@H]3CC[C@@H](OC(=O)N4CC5CC4C5)C3)[nH]n2)c1] | ¹H NMR (400 MHz, DMSO-D6) δ 12.32 (s, 1H), 10.55 (s, 1H), 10.15 (s, 1H), 6.32 (s, 1H), 6.14-6.15 (m, 1H), 6.06-6.07 (m, 1H), 5.03-5.04 (m, 1H), 4.81 (s, 2H), 4.20-4.22 (m, 1H), 3.81 (s, 3H), 3.20 (s, 2H), 3.09-3.13 (m, 1H), 2.76 (s, 1H), |
| | | 2.41-2.43 (m, 1H), 2.01-2.03 (m, |
| | | 1H), 1.69-1.89 (m, 6H), 1.22-1.23 |
| | | (m, 2H). MS (ESI) calcd. for |
| | | C₂₄H₂₈N₄O₇, 484.20 m/z, found |
| | | [M + H]⁺ 485.30 m/z. |
| 229 | 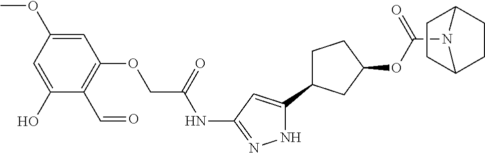 [SMILES: COc1cc(O)c(C=O)c(OCC(=O)Nc2cc([C@H]3CC[C@@H](OC(=O)N4C5CCC4CC5)C3)[nH]n2)c1] | ¹H NMR (400 MHz, DMSO-D6) δ 12.33 (br s, 1H), 12.17 (s, 1H), 10.54 (s, 1H), 10.15 (s, 1H), 6.35 (s, 1H), 6.14-6.15 (m, 1H), 6.06- 6.07 (m, 1H), 5.00-5.01 (m, 1H), 4.81 (s, 2H), 4.06-4.07 (m, 2H), 3.82 (s, 3H), 3.10-3.13 (m, 1H), 2.39-2.49 (m, 1H), 2.01-2.03 (m, 1H), 1.89-1.90 (m, 1H), 1.55-1.77 (m, 7H), 1.35-1.37 (m, 4H). MS |
| | | (ESI) calcd. for C₂₅H₃₀N₄O₇, |
| | | 498.21 m/z, found [M + H]⁺ |
| | | 499.25 m/z. |
| 230 | 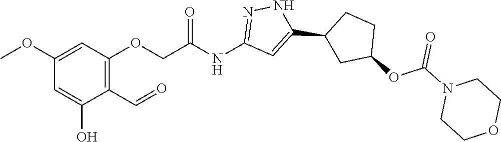 [SMILES: COc1cc(O)c(C=O)c(OCC(=O)Nc2cc([C@H]3CC[C@@H](OC(=O)N4CCOCC4)C3)[nH]n2)c1] | ¹H NMR (400 MHz, DMSO-D6) δ (ppm): 12.33 (s, 1H), 12.17 (br s, 1H), 10.54 (s, 1H), 10.15 (s, 1H), 6.32 (s, 1H), 6.14 (s, 1H), 6.07 (s, 1H), 5.03-5.05 (m, 1H), 4.80 (s, 2H), 3.81 (s, 3H), 3.45-3.55 (m, 4H), 3.27-3.30 (m, 4H), 3.09-3.17 (m, 1H), 2.36-2.44 (m, 1H), 1.99- 2.06 (m, 1H), 1.85-1.95 (m, 1H), |
| | | 1.69-1.80 (m, 3H). MS (ESI) |
| | | calcd. for C₂₃H₂₈N₄O₈, 488.19 |
| | | m/z, found [M + H]⁺ 489.15 m/z. |
| 232 | 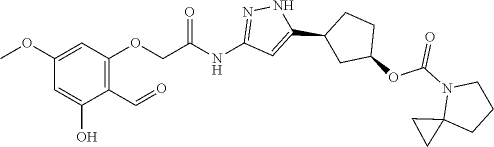 [SMILES: COc1cc(O)c(C=O)c(OCC(=O)Nc2cc([C@H]3CC[C@@H](OC(=O)N4CCCC45CC5)C3)[nH]n2)c1] | ¹H NMR (400 MHz, DMSO-D6) δ 12.32 (br s, 1H), 12.17 (s, 1H), 10.54 (s, 1H), 10.15 (s, 1H), 6.33 (s, 1H), 6.07-6.14 (m, 1H), 4.94- 4.95 (m, 1H), 4.81 (s, 1H), 3.81 (s, 3H), 3.41-3.42 (m, 2H), 3.35-3.39 (m, 1H), 2.37-2.39 (m, 1H), 1.81- 1.84 (m, 1H), 1.71-1.78 (m, 8H), 1.59-1.61 (m, 2H), 0.44-0.50 (m, |
| | | 2H). MS(ESI) calcd. for |
| | | C₂₅H₃₀N₄O₇, 498.21 m/z, found |
| | | [M + H]⁺ 499.35 m/z. |
| 233 | 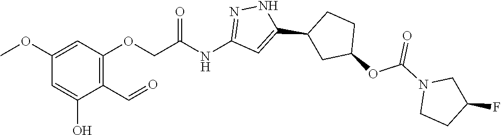 [SMILES: COc1cc(O)c(C=O)c(OCC(=O)Nc2cc([C@H]3CC[C@@H](OC(=O)N4CC[C@H](F)C4)C3)[nH]n2)c1] | ¹H NMR (400 MHz, DMSO-D6) δ (ppm): 12.31-12.35 (m, 1H), 12.18 (s, 1H), 10.53-10.58 (m, 1H), 10.14 (s, 1H), 6.33 (s, 1H), 6.14 (s, 1H), 6.06 (s, 1H), 5.18-5.31 (m, 1H), 5.03-5.04 (m, 1H), 4.80 (s, 2H), 3.80 (s, 3H), 3.40-3.51 (m, 3H), 3.25-3.31 (m, 1H), 3.10-3.12 (m, 1H), 2.41-2.43 (m, 1H), 1.88- |
| | | 2.08 (m, 4H), 1.69-1.79 (m, 3H). |
| | | MS (ESI) calcd. for C₂₃H₂₇FN₄O₇, |
| | | 490.19 m/z, found [M + H]⁺ |
| | | 491.10 m/z. |
| 235 | 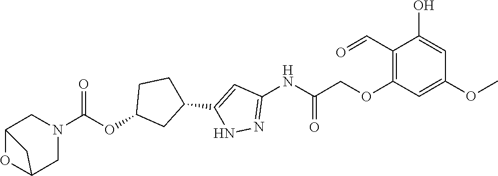 [SMILES: COc1cc(O)c(C=O)c(OCC(=O)Nc2cc([C@H]3CC[C@@H](OC(=O)N4CC5CC(C4)O5)C3)[nH]n2)c1] | ¹H NMR (400 MHz, DMSO-D6) δ (ppm): 12.32 (s, 1H), 12.17 (s, 1H), 10.53 (s, 1H), 10.15 (s, 1H), 6.35-6.37 (m, 1H), 6.15 (d, J = 1.6 Hz, 1H), 6.07 (d, J = 1.6 Hz, 1H), 5.10-5.12 (m, 1H), 4.81 (s, 2H), 4.55 (s, 1H), 4.42-4.47 (m, 1H), 3.81 (s, 3H), 3.45-3.52 (m, 4H), 3.12-3.17 (m, 1H), 2.97-3.03 (m, 1H), 2.41-2.47 (m, 1H), 2.02-2.08 (m, 1H), 1.90-1.95 (m, 1H), 1.72- |
| | | 1.84 (m, 4H). MS (ESI) calcd. for |
| | | C₂₄H₂₈N₄O₈, 500.19 m/z, found |
| | | [M + H]⁺ 501.20 m/z. |
| 236 | 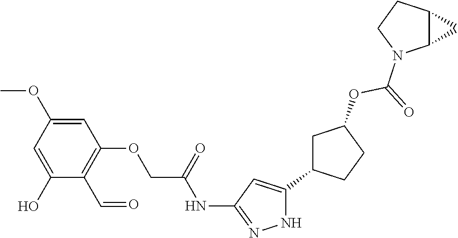 [SMILES: COc1cc(O)c(C=O)c(OCC(=O)Nc2cc([C@H]3CC[C@@H](OC(=O)N4CC[C@@H]5C[C@@H]54)C3)[nH]n2)c1] | ¹H NMR (400 MHz, DMSO-D6) δ 12.33 (s, 1H), 12.18 (s, 1H), 10.55 (s, 1H), 10.15 (s, 1H), 6.34-6.35 (m, 1H), 6.14-6.15 (m, 1H), 6.07- 6.08 (m, 1H), 5.03-5.04 (m, 1H), 4.81 (s, 2H), 3.81 (s, 3H), 3.51- 3.54 (m, 1H), 3.31-3.34 (m, 1H), 3.08-3.14 (m, 1H), 2.87-2.91 (m, 1H), 2.50-2.51 (m, 1H), 1.98-2.04 (m, 2H), 1.60-1.80 (m, 5H), 1.49- 1.50 (m, 1H), 0.59-0.61 (m, 1H), 0.50-0.51 (m, 1H). MS (ESI) calcd. for C₂₄H₂₈N₄O₇, 484.20 m/z, found [M + H]⁺ 485.10 m/z. |
| 237 | 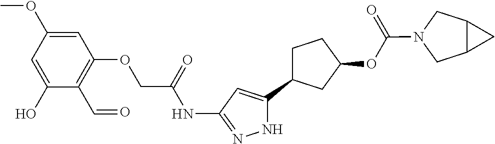 [SMILES: COc1cc(O)c(C=O)c(OCC(=O)Nc2cc([C@H]3CC[C@@H](OC(=O)N4CC5CC5C4)C3)[nH]n2)c1] | ¹H NMR (400 MHz, DMSO-D6) δ (ppm): 12.32 (br s, 1H), 12.17 (s, 1H), 10.55 (s, 1H), 10.15 (s, 1H), 6.32 (s, 1H), 6.13-6.15 (m, 1H), 6.07 (s, 1H), 4.95-5.01 (m, 1H), 4.81 (s, 2H), 3.80 (s, 3H), 3.39- 3.43 (m, 2H), 3.26-3.28 (m, 2H), 3.05-3.11 (m, 1H), 2.36-2.44 (m, 1H), 1.98-2.04 (m, 1H), 1.84-1.89 |
| | | (m, 1H), 1.63-1.76 (m, 3H), 1.45- |
| | | 1.48 (m, 2H), 0.62-0.65 (m, 1H), |
| | | 0.02-0.03 (m, 1H). MS (ESI) |
| | | calcd. for C₂₄H₂₈N₄O₇, 484.20 |
| | | m/z, found [M + H]⁺ 485.20 m/z. |
| 238 | 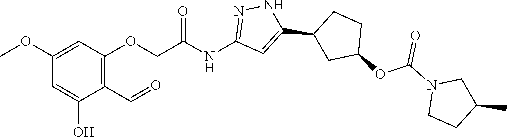 | ¹H NMR (300 MHz, DMSO-D6) δ 12.32 (br s, 1H), 10.55 (s, 1H), 10.15 (s, 1H), 6.34 (d, J = 3.9 Hz, 1H), 6.15 (d, J = 2.1 Hz, 1H), 6.08 (d, J = 2.1 Hz, 1H), 5.00-5.01 (m, 1H), 4.81 (s, 2H), 3.81 (s, 3H), 3.31-3.43 (m, 2H), 3.08-3.17 (m, 2H), 2.71-2.77 (m, 1H), 2.30-2.39 (m, 1H), 2.01-2.15 (m, 2H), 1.81- |
| | | 1.87 (m, 2H), 1.67-1.79 (m, 3H), |
| | | 1.37-1.42 (m, 1H), 0.92-0.97 (m, |
| | | 3H). MS (ESI) calcd. for |
| | | C₂₄H₃₀N₄O₇, 486.21 m/z, found |
| | | [M + H]⁺ 487.25 m/z. |
| 240 | 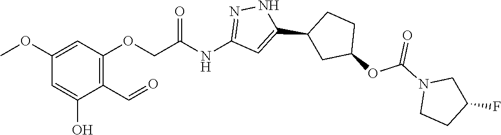 [SMILES: COc1cc(O)c(C=O)c(OCC(=O)Nc2cc([C@H]3CC[C@@H](OC(=O)N4CC[C@@H](F)C4)C3)[nH]n2)c1] | ¹H NMR (300 MHz, DMSO-D6) δ 12.33 (br s, 1H), 10.57 (br s, 1H), 10.15 (s, 1H), 6.33 (d, J = 9.6 Hz, 1H), 6.15 (d, J = 1.8 Hz, 1H), 6.07 (d, J = 2.1 Hz, 1H), 5.30-5.36 (m, 1H), 5.05-5.18 (m, 1H), 4.81 (s, 2H), 3.78 (s, 3H), 3.20-3.52 (m, 4H), 3.00-3.20 (m, 1H), 2.35-2.46 (m, 1H), 1.86-2.03 (m, 4H), 1.65- |
| | | 1.84 (m, 3H). 19F NMR (300 |
| | | MHz, DMSO-D6) δ-176.48. MS |
| | | (ESI) calcd. for C₂₃H₂₇FN₄O₇, |
| | | 490.19 m/z, found [M + H]⁺ |
| | | 491.15 m/z. |
| 241 | 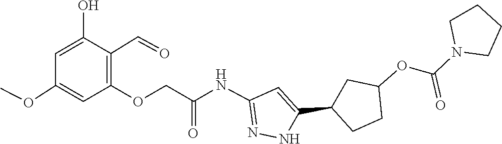 [SMILES: COc1cc(O)c(C=O)c(OCC(=O)Nc2cc([C@@H]3CCC(OC(=O)N4CCCC4)C3)[nH]n2)c1] | ¹H NMR (400 MHz, DMSO-D6) δ 10.15 (s, 1H), 6.32 (s, 1H), 6.07- 6.15 (m, 2H), 5.00-5.02 (m, 1H), 4.81 (s, 2H), 3.80 (s, 3H), 3.16- 3.21 (m, 4H), 3.09-3.13 (m, 1H), 2.32-2.40 (m, 1H), 2.02-2.04 (m, 1H), 1.84-1.91 (m, 1H), 1.69-1.78 (m, 7H). MS (ESI) calcd. for C₂₃H₂₈N₄O₇: 472.20 m/z, found |
| | | [M + H]⁺ 473.15 m/z. |
| 242 | 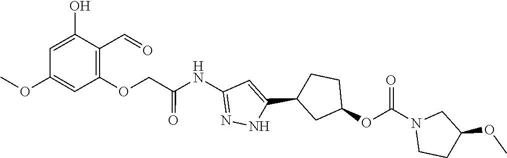 | ¹H NMR (400 MHz, DMSO-D6) δ 12.33 (s, 1H), 10.57 (s, 1H), 10.15 (s, 1H), 6.33 (d, J = 11.6 Hz, 1H), 6.14 (d, J = 2 Hz, 1H), 6.07 (d, J = 2 Hz, 1H), 4.97-5.05 (m, 1H), 4.81 (s, 2H), 3.85-3.88 (m, 1H), 3.81(s, 3H), 3.25-29 (m, 3H), 3.16-3.21 (m, 4H), 3.09-3.13 (m, 1H), 2.36- 2.45 (m, 1H), 1.99-2.04 (m, 1H), 1.82-1.87 (m, 3H), 1.66-1.78 (m, |
| | | 3H). MS (ESI) calcd. for |
| | | C₂₄H₃₀N₄O₈, 502.21 m/z, found |
| | | [M + H]⁺ 503.15 m/z. |
| 243 | 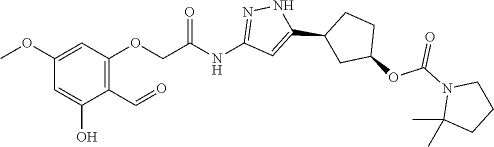 [SMILES: COc1cc(O)c(C=O)c(OCC(=O)Nc2cc([C@H]3CC[C@@H](OC(=O)N4CCCC4(C)C)C3)[nH]n2)c1] | ¹H NMR (300 MHz, DMSO-D6) δ 12.32 (br s, 1H), 10.55 (br s, 1H), 10.15 (s, 1H), 6.31 (s, 1H), 6.14 (s, 1H), 6.07 (s, 1H), 4.98-5.05 (m, 1H), 4.81 (s, 2H), 3.80 (s, 3H), 3.29-3.34 (m, 2H), 3.07-3.12 (m, 1H), 2.49-2.50 (m, 1H), 2.01-2.06 (m, 1H), 1.86-1.98 (m, 1H), 1.68- 1.82 (m, 7H), 1.23-1.29 (m, 6H). |
| | | MS (ESI) calcd. for C₂₅H₃₂N₄O₇, |
| | | 500.23 m/z, found [M + H]⁺ |
| | | 501.25 m/z. |
| 245 | 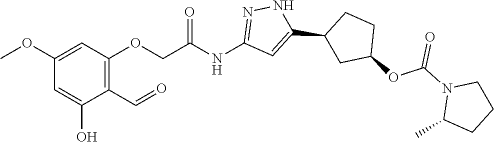 [SMILES: COc1cc(O)c(C=O)c(OCC(=O)Nc2cc([C@H]3CC[C@@H](OC(=O)N4CCC[C@@H]4C)C3)[nH]n2)c1] | ¹H NMR (400 MHz, DMSO-D6) δ 12.32 (s, 1H), 10.55 (s, 1H), 10.15 (s, 1H), 6.32 (s, 1H), 6.14 (d, J = 2 Hz, 1H), 6.07 (d, J = 2.4 Hz, 1H), 4.98-5.05 (m, 1H), 4.81 (s, 2H), 3.81-3.83 (m, 4H), 3.22-3.25 (m, 2H), 3.06-3.14 (m, 1H), 2.33-2.47 (m, 1H), 1.99-2.05 (m, 1H), 1.87- 1.90 (m, 2H), 1.61-1.80 (m, 5H), |
| | | 1.46-1.48 (m, 1H), 1.01-1.09 (m, |
| | | 3H). MS (ESI) calcd. for |
| | | C₂₄H₃₀N₄O₇, 486.21 m/z, found |
| | | [M + H]⁺ 487.10 m/z. |
| 246 | 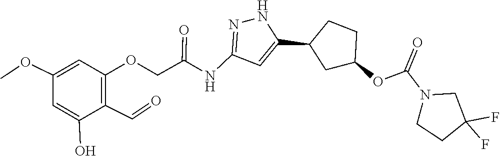 [SMILES: COc1cc(O)c(C=O)c(OCC(=O)Nc2cc([C@H]3CC[C@@H](OC(=O)N4CCC(F)(F)C4)C3)[nH]n2)c1] | ¹H NMR (400 MHz, DMSO-D6) δ (ppm): 12.33 (s, 1H), 10.58 (s, 1H), 10.15 (s, 1H), 6.34 (s, 1H), 6.14 (s, 1H), 6.07 (s, 1H), 5.05- 5.06 (m, 1H), 4.81 (s, 2H), 3.81 (s, 3H), 3.61-3.68 (m, 2H), 3.45-3.48 (m, 2H), 3.10-3.14 (m, 1H), 2.36- 2.45 (m, 3H), 2.00-2.08 (m, 1H), 1.85-1.95 (m, 1H), 1.69-1.82 (m, 3H). MS (ESI) calcd. for |
| | | C₂₃H₂₆F₂N₄O₇ 508.18 m/z, found |
| | | [M + H]⁺ 509.20 m/z. |
| 247 | 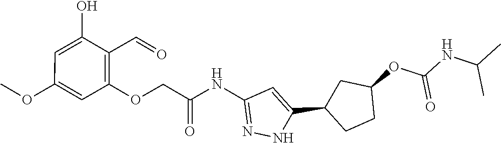 [SMILES: COc1cc(O)c(C=O)c(OCC(=O)Nc2cc([C@@H]3CC[C@H](OC(=O)NC(C)C)C3)[nH]n2)c1] | ¹H NMR (400 MHz, DMSO-D6) δ 10.15 (s, 1H), 6.30 (s, 1H), 6.14 (s, 1H), 6.07 (s, 1H), 4.98 (s, 1H), 4.81 (s, 2H), 3.81(s, 3H), 3.60-3.63 (m, 1H), 3.03-3.08 (m, 1H), 2.33- 2.48 (m, 1H), 1.97-2.02 (m, 1H), 1.85-1.90 (m, 1H), 1.49-1.76 (m, 3H), 0.98-1.05 (m, 6H). MS (ESI) calcd. for C₂₂H₂₈N₄O₇: 460.20, |
| | | found [M + H]⁺ 461.10 m/z. |
| 248 | 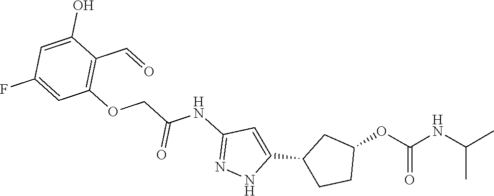 [SMILES: CC(C)NC(=O)O[C@@H]1CC[C@H](c2cc(NC(=O)COc3cc(F)cc(O)c3C=O)n[nH]2)C1] | ¹H NMR (300 MHz, DMSO-D6) δ 10.57 (s, 1H), 10.21 (s, 1H), 8.35 (s, 1H), 6.94 (s, 1H), 6.39 (s, 1H), 6.25 (d, J = 8.6 Hz, 2H), 5.00 (s, 1H), 4.79 (s, 2H), 3.56 (s, 2H), 3.06 (s, 1H), 2.01 (d, J = 7.6 Hz, 1H), 1.89 (s, 1H), 1.72 (d, J = 9.4 Hz, 2H), 1.60 (s, 1H), 1.02 (d, J = 6.6 Hz, 6H). 19F NMR (282 MHz, DMSO-D6) δ −97.841. MS (ESI) calcd. for C₂₁H₂₅FN₄O₆, 448.18 m/z, found [M + H]⁺ 449.15 m/z. |
| 250 | 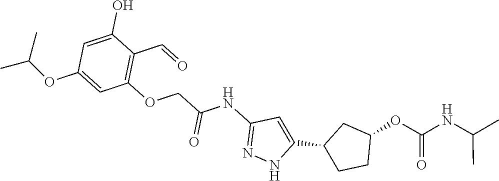 [SMILES: CC(C)NC(=O)O[C@@H]1CC[C@H](c2cc(NC(=O)COc3cc(OC(C)C)cc(O)c3C=O)n[nH]2)C1] | ¹H NMR (400 MHz, DMSO-D6) δ 12.34-12.29 (m, 1H), 12.24-12.09 (m, 1H), 10.60-10.50 (m, 1H), 10.17-10.09 (m, 1H), 6.95 (d, J = 7.8 Hz, 1H), 6.35-6.27 (m, 1H), 6.13 (d, J = 2.1 Hz, 1H), 6.01 (d, J = 2.1 Hz, 1H), 5.05-4.93 (m, 1H), 4.85-4.76 (m, 2H), 4.77-4.68 (m, 1H), 3.63-3.52 (m, 1H), 3.13-2.99 (m, 1H), 2.46-2.41 (m, 1H), 2.06- 1.84 (m, 2H), 1.79-1.63 (m, 2H), |
| | | 1.60-1.52 (m, 1H), 1.27 (d, J = 6.0 |
| | | Hz, 6H), 1.02 (d, J = 6.6, 2.2 Hz, |
| | | 6H). MS (ESI) mass calcd. for |
| | | C₂₄H₃₂N₄O₇ 488.23 m/z, found |
| | | [M + H]⁺ 489.25 m/z. |
| 252 | 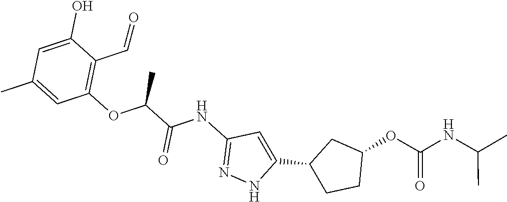 [SMILES: Cc1cc(O)c(C=O)c(O[C@@H](C)C(=O)Nc2cc([C@H]3CC[C@@H](OC(=O)NC(C)C)C3)[nH]n2)c1] | ¹H NMR (300 MHz, DMSO-D6) δ 11.78 (s, 1H), 10.60 (s, 1H), 10.31 (s, 1H), 6.93 (d, J = 7.9 Hz, 1H), 6.38 (s, 1H), 6.31 (d, J = 6.4 Hz, 2H), 5.01-4.82 (m, 2H), 3.63-3.50 (m, 2H), 3.09-2.98 (m, 1H), 2.50- 2.37 (m, 1H), 2.25 (s, 3H), 2.11- 1.78 (m, 1H), 1.77-1.62 (m, 1H), 1.60 (s, 2H), 1.56 (d, J = 6.5 Hz, 3H), 1.02 (d, J = 6.5 Hz, 6H). MS (ESI) calcd. for C₂₃H₃₀N₄O₆, 458.22 m/z, found [M + H]⁺ 459.15 m/z. |
| 253 | 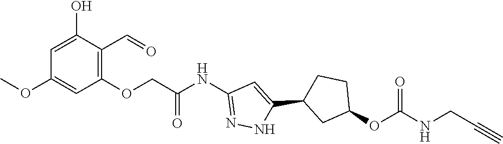 [SMILES: C#CCNC(=O)O[C@@H]1CC[C@H](c2cc(NC(=O)COc3cc(OC)cc(O)c3C=O)n[nH]2)C1] | ¹H NMR (400 MHz, DMSO-D6) δ (ppm): 12.32 (s, 1H), 12.18 (s, 1H), 10.54 (s, 1H), 10.15 (s, 1H), 7.53-7.56 (m, 1H), 6.31 (s, 1H), 6.14 (d, J = 1.8 Hz, 1H), 6.06 (d, J = 2.1 Hz, 1H), 5.01-5.02 (m, 1H), 4.81 (s, 2H), 3.81 (s, 3H), 3.73- 3.75 (m, 2H), 3.02-3.07 (m, 2H), 2.43-2.46 (m, 1H), 1.87-2.04 (m, |
| | | 2H), 1.54-1.74 (m, 3H). MS (ESI) |
| | | calcd. for C₂₂H₂₄N₄O₇, 456.16 m/z, |
| | | found [M + H]⁺ 457.15 m/z. |
| 254 | 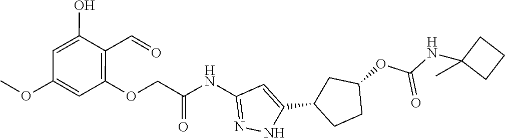 [SMILES: COc1cc(O)c(C=O)c(OCC(=O)Nc2cc([C@H]3CC[C@@H](OC(=O)NC4(C)CCC4)C3)[nH]n2)c1] | ¹H NMR (400 MHz, DMSO-D6) δ 12.31 (s, 1H), 10.56 (s, 1H), 10.15 (s, 1H), 7.10-7.18 (m, 1H), 6.34 (s, 1H), 6.16 (s, 1H), 6.06 (s, 1H), 4.95-5.03 (m, 1H), 4.82 (s, 2H), 3.83 (s, 3H), 3.00-3.09 (m, 1H), 2.44-2.49 (m, 1H), 2.16-2.26 (m, 2H), 1.96-2.02 (m, 1H), 1.96-2.04 (m, 1H), 1.54-1.92 (m, 7H), 1.32 |
| | | (m, 3H). MS (ESI) calcd. for |
| | | C₂₄H₃₀N₄O₇ 486.21 m/z, found |
| | | [M + H]⁺ 487.35 m/z. |
| 256 | 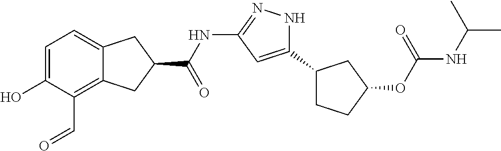 [SMILES: CC(C)NC(=O)O[C@@H]1CC[C@H](c2cc(NC(=O)[C@@H]3Cc4ccc(O)c(C=O)c4C3)n[nH]2)C1] | ¹H NMR (300 MHz, DMSO-D6) δ 12.06 (s, 1H), 10.59 (s, 1H), 10.41 (s, 1H), 10.35 (s, 1H), 7.35 (d, J = 8.3 Hz, 1H), 6.95 (d, J = 8.1 Hz, 1H), 6.80 (d, J = 8.3 Hz, 1H), 6.34 (s, 1H), 4.99 (s, 1H), 3.54-3.49 (m, 1H), 3.44 (s, 2H), 3.36 (s, 1H), 2.99 (d, J = 8.4 Hz, 2H), 2.45 (d, J = 7.1 Hz, 1H), 2.04-1.93 (m, 1H), 1.88 (s, 1H), 1.71 (d, J = 8.9 Hz, |
| | | 2H), 1.59 (s, 1H), 1.24 (s, 1H), |
| | | 1.02 (d, J = 6.5 Hz, 6H). MS (ESI) |
| | | calcd. for C₂₃H₂₈N₄O₅, 440.21 |
| | | m/z, found [M + H]⁺ 441.25 m/z. |
| 257 | 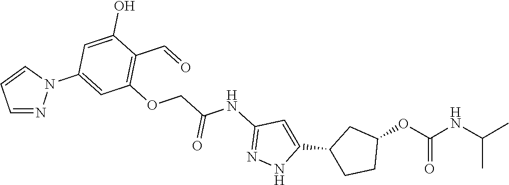 [SMILES: CC(C)NC(=O)O[C@@H]1CC[C@H](c2cc(NC(=O)COc3cc(-n4cccn4)cc(O)c3C=O)n[nH]2)C1] | ¹H NMR (400 MHz, DMSO-D6) δ 12.06 (s, 1H), 10.58 (s, 1H), 10.32 (s, 1H), 8.70 (d, J = 2.4Hz, 1H), 7.84 (s, 1H), 7.12 (s, 2H), 6.93- 6.95 (m, 1H), 6.63-6.64 (m, 1H), 6.32 (s, 1H), 4.95-5.00 (m, 3H), 3.52-3.53 (m, 1H), 3.02-3.07 (m, 1H), 2.49-2.50 (m, 1H), 1.99-2.01 (m, 1H), 1.86-1.90 (m, 1H), 1.58- 1.72 (m, 3H), 1.01-1.03 (m, 6H). MS (ESI) calcd. For C₂₄H₂₈N₆O₆, 496.21 m/z, found [M + H]⁺ |
| | | 497.15 m/z. |
| 258 | 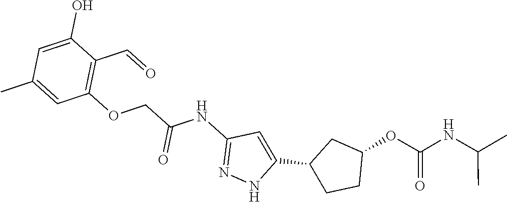 [SMILES: Cc1cc(O)c(C=O)c(OCC(=O)Nc2cc([C@H]3CC[C@@H](OC(=O)NC(C)C)C3)[nH]n2)c1] | ¹H NMR (300 MHz, DMSO-D6) δ (ppm): 11.48 (s, 1H), 10.33 (s, 1H), 10.22 (s, 1H), 6.94 (d, J = 7.8 Hz, 1H), 6.64 (s, 2H), 6.26 (s, 1H), 4.97 (br s, 1H), 3.52-3.62 (m, 1H), 3.14 (t, J = 7.7 Hz, 2H), 3.01 (t, J = 8.6 Hz, 1H), 2.54-2.57 (m, 1H), 2.39-2.44 (m, 1H), 2.24 (s, 3H), 1.94-2.02 (m, 1H), 1.80-1.90 (m, 1H), 1.65-1.71 (m, 2H), 1.49-1.53 (m, 1H), 1.02 (d, J = 6.6 Hz, 6H). MS (ESI) calcd. for C₂₃H₃₀N₄O₅, |
| | | 442.22 m/z, found [M + H]⁺ |
| | | 443.20 m/z. |
| 259 | 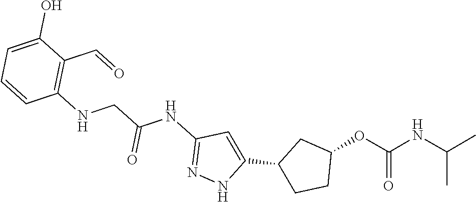 [SMILES: CC(C)NC(=O)O[C@@H]1CC[C@H](c2cc(NC(=O)CNc3cccc(O)c3C=O)n[nH]2)C1] | ¹H NMR (300 MHz, DMSO-D6) δ (ppm): 12.09 (s, 1H), 10.46 (s, 1H), 10.31 (s, 1H), 9.02 (s, 1H), 7.19 (t, J = 8.2 Hz, 1H), 6.94 (d, J = 8.1 Hz, 1H), 6.35 (s, 1H), 6.08 (d, J = 8.1 Hz, 1H), 5.93 (d, J = 8.3 Hz, 1H), 5.00 (s, 1H), 3.98 (d, J = 5.3 Hz, 2H), 3.55 (dd, J = 3.7, 7.1 Hz, 1H), 3.11-2.99 (m, 1H), 1.89- 1.82 (m, 3H), 1.8-1.5 (m, 4H), 1.08 (s, 3H), 0.99 (s, 3H). MS (ESI) calcd. for C₂₁H₂₇N₅O₅ 429.20 m/z, found [M + H]⁺ 430.15 m/z. |

### Example 21 Synthetic Procedures for Selected Prodrug Compounds

### Example 21.1: Preparation of Compound 128b: (1R,3S)-3-(3-(2-(3-hydroxy-5-methoxy-2-((E)-(propylimino)methyl)phenoxy)acetamido)-1H-pyrazol-5-yl)cyclopentyl tert-butylcarbamate

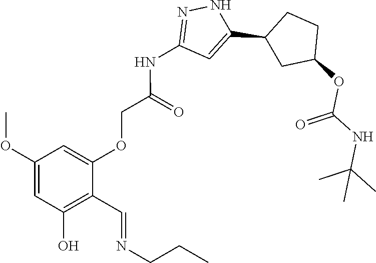 [SMILES: CCC/N=C/c1c(O)cc(OC)cc1OCC(=O)Nc1cc([C@H]2CC[C@@H](OC(=O)NC(C)(C)C)C2)[nH]n1]

### Synthetic Route

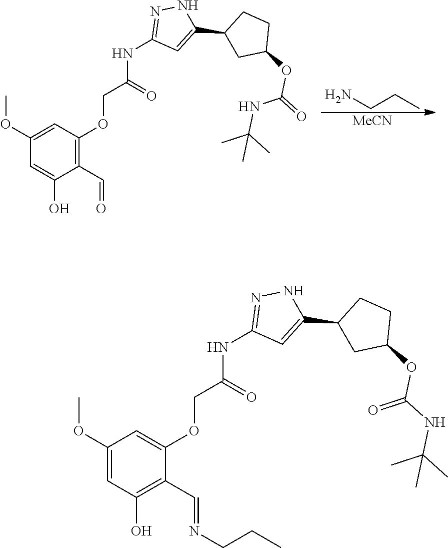

### Step 1: Synthesis of (1R,3S)-3-(3-(2-(3-hydroxy-5-methoxy-2-((E)-(propylimino)methyl)phenoxy)acetamido)-1H-pyrazol-5-yl)cyclopentyl tert-butylcarbamate

(1R,3S)-3-{5-[2-(2-formyl-3-hydroxy-5-methoxyphenoxy)acetamido]-2H-pyrazol-3-yl}cyclopentyl N-tert-butylcarbamate (500 mg, 1.054 mmol, 1 equiv), propylamine (311.43 mg, 5.270 mmol, 5 equiv) and ACN (6 mL) were added to a 40 mL vial and stirred until homogeneous. The reaction mixture was stirred at rt for 2 h, then concentrated to dryness under vacuum to afford a yellow. The oil was purified by reverse-phase chromatography (5-50% MeCN/H₂O) to afford (1R,3S)-3-[5-(2-{3-hydroxy-5-methoxy-2-[(1E)-(propylimino)methyl]phenoxy}acetamido)-2H-pyrazol-3-yl]cyclopentyl N-tert-butylcarbamate (436.8 mg, 80.40%) as a yellow solid. 1H NMR (400 MHz, DMSO-d6) δ 14.37-14.24 (m, 1H), 12.30-11.99 (m, 1H), 10.59-10.40 (m, 1H), 8.54 (d, J=7.8 Hz, 1H), 6.92-6.67 (m, 1H), 6.50-6.20 (m, 1H), 5.76 (d, J=2.1 Hz, 1H), 5.58 (d, J=2.1 Hz, 1H), 5.09-4.94 (m, 1H), 4.88-4.39 (m, 2H), 3.75-3.61 (m, 3H), 3.55-3.48 (m, 2H), 3.09-2.99 (m, 1H), 2.04-1.97 (m, 1H), 1.92-1.84 (m, 1H), 1.75-1.55 (m, 5H), 1.26-1.16 (m, 8H), 0.93 (t, J=7.4 Hz, 3H). LCMS (ESI): mass calcd. for C₂₆H₃₇N₅O₆, 515.61; m/z found 516.35 [M+H]⁺.

### Example 21.2: Preparation of compound 172a: (1R,3S)-3-(3-(2-(3-hydroxy-2-((E)-(isopropylimino)methyl)-5-methoxyphenoxy)acetamido)-1H-pyrazol-5-yl)cyclopentyl 1,2,2-trimethylhydrazine-1-carboxylate

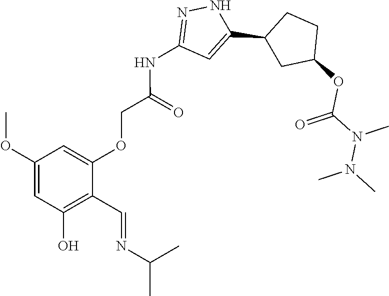 [SMILES: COc1cc(O)c(/C=N/C(C)C)c(OCC(=O)Nc2cc([C@H]3CC[C@@H](OC(=O)N(C)N(C)C)C3)[nH]n2)c1]

### Synthetic Route

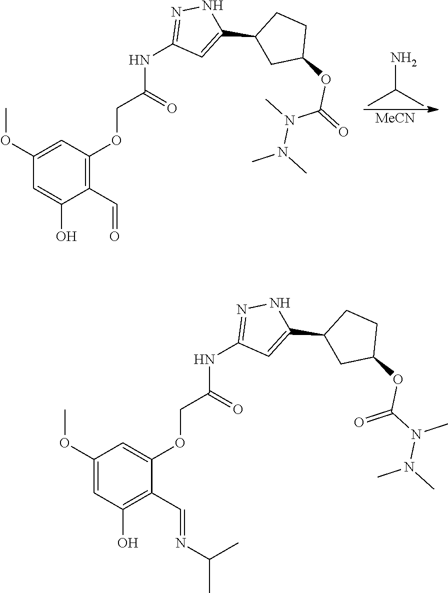

### Step 1: Synthesis of (1R,3S)-3-(3-(2-(3-hydroxy-2-((E)-(isopropylimino)methyl)-5-methoxyphenoxy)acetamido)-1H-pyrazol-5-yl)cyclopentyl 1,2,2-trimethylhydrazine-1-carboxylate

To a stirred solution of 2-(2-formyl-3-hydroxy-5-methoxyphenoxy)-N-{5-[(1S,3R)-3-[(N,N′,N′-trimethylhydrazinecarbonyl)oxy]cyclopentyl]-1H-pyrazol-3-yl}acetamide (70 mg, 0.147 mmol, 1 equiv) in anhydrous MeCN (5 mL) was added isopropylamine (43.51 mg, 0.735 mmol, 5 equiv) at room temperature. The reaction mixture was stirred at room temperature for 1 h. After completion of reaction, the resulting mixture was concentrated under reduced pressure. The residue was purified by C18 reverse phase chromatography, 5 to 65% MeCN/H₂O to afford 2-{3-hydroxy-2-[(1E)-(isopropylimino)methyl]-5-methoxyphenoxy}-N-{5-[(1S,3R)-3-[(N,N′,N′-trimethylhydrazinecarbonyl)oxy]cyclopentyl]-1H-pyrazol-3-yl}acetamide (14.3 mg, 18.1% yield). 1H NMR (400 MHz, DMSO-d6) δ 14.45 (br s, 1H), 12.20 (s, 1H), 10.50 (s, 1H), 8.59 (s, 1H), 6.34 (s, 1H), 5.78 (s, 1H), 5.61 (s, 1H), 5.05-4.98 (m, 1H), 4.68 (s, 2H), 3.79-3.65 (m, 7H), 3.22-3.14 (m, 1H), 2.82 (m, 3H), 2.42-2.37 (m, 4H), 2.10-1.99 (m, 1H), 1.87-1.67 (m, 4H), 1.30-1.23 (m, 6H). MS (ESI): calcd. for C₂₅H₃₆N₆O₆ 516.27 m/z; found [M+H]⁺ 517.40 m/z.

### Example 21.3: Preparation of Compound 251b: (1R,3S)-3-(3-(2-(3-hydroxy-5-methoxy-2-((E)-(propylimino)methyl)phenoxy)acetamido)-1H-pyrazol-5-yl)cyclopentyl (1-methylcyclopropyl)carbamate

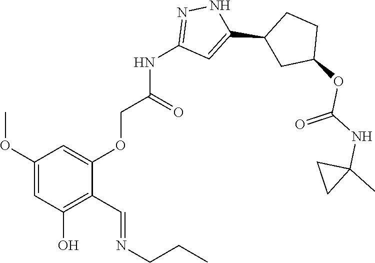 [SMILES: CCC/N=C/c1c(O)cc(OC)cc1OCC(=O)Nc1cc([C@H]2CC[C@@H](OC(=O)NC3(C)CC3)C2)[nH]n1]

### Synthetic Route

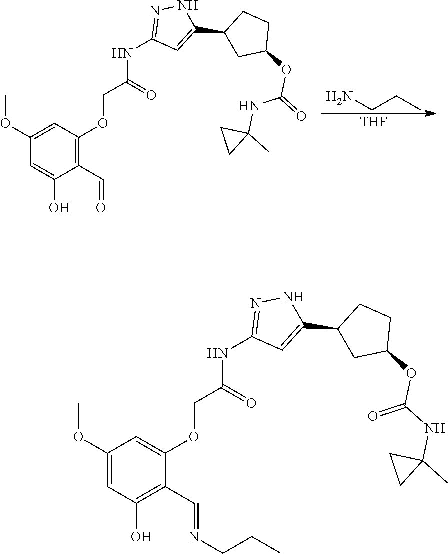

### Step 1: Synthesis of (1R,3S)-3-(3-(2-(3-hydroxy-5-methoxy-2-((E)-(propylimino)methyl)phenoxy)acetamido)-1H-pyrazol-5-yl)cyclopentyl (1-methylcyclopropyl)carbamate

(1R,3S)-3-(3-(2-(2-formyl-3-hydroxy-5-methoxyphenoxy)acetamido)-1H-pyrazol-5-yl)cyclopentyl (1-methylcyclopropyl)carbamate (80 mg, 0.169 mmol, 1 equiv), propan-1-amine (20.02 mg, 0.338 mmol, 2 equiv) and THF (2 mL) were added to a 20 mL reaction flask and stirred at rt for 1 h. The resulting mixture was purified by reverse phase HPLC from 22 to 42% with ACN/H₂O (10 mmol/L NH₄HCO₃ as modifier) to afford (1R,3S)-3-(3-(2-(3-hydroxy-5-methoxy-2-((E)-(propylimino)methyl)phenoxy)acetamido)-1H-pyrazol-5-yl)cyclopentyl (1-methylcyclopropyl)carbamate (27.8 mg, 30.98% yield) as a light yellow solid. 1H NMR (400 MHz, DMSO) δ 14.31 (s, 1H), 12.16 (s, 1H), 10.49 (s, 1H), 8.53 (d, J=6.9 Hz, 1H), 7.36 (s, 1H), 6.29 (s, 1H), 5.75 (d, J=2.1 Hz, 1H), 5.56 (d, J=2.1 Hz, 1H), 4.97 (s, 1H), 4.67 (s, 2H), 3.50 (td, J=7.0, 2.4 Hz, 3H), 3.33 (s, 2H), 3.07-2.98 (m, 1H), 2.49-2.40 (m, 1H), 2.00 (s, 1H), 1.92-1.84 (m, 1H), 1.79-1.48 (m, 5H), 1.22 (s, 3H), 0.92 (t, J=7.4 Hz, 3H), 0.58 (s, 2H), 0.47-0.43 (m, 2H), MS (ESI) calcd. for C₂₆H₃₅N₅O₆, 513.25 m/z, found [M+H]⁺ 514.20 m/z.

### Example 21.4: Preparation of Compound 251d: (1R,3S)-3-(3-(2-(3-hydroxy-2-((E)-((2-hydroxyethyl)imino)methyl)-5-methoxyphenoxy)acetamido)-1H-pyrazol-5-yl)cyclopentyl (1-methylcyclopropyl)carbamate

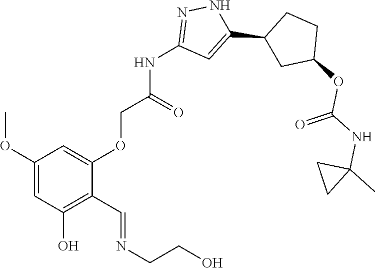 [SMILES: COc1cc(O)c(/C=N/CCO)c(OCC(=O)Nc2cc([C@H]3CC[C@@H](OC(=O)NC4(C)CC4)C3)[nH]n2)c1]

### Synthetic Route

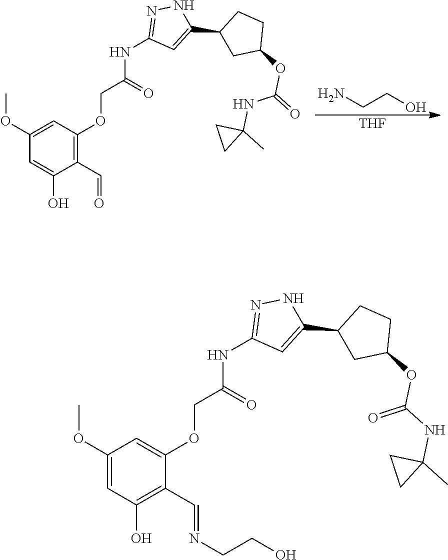

### Step 1: Synthesis of (1R,3S)-3-(3-(2-(3-hydroxy-2-((E)-((2-hydroxyethyl)imino)methyl)-5-methoxyphenoxy)acetamido)-1H-pyrazol-5-yl)cyclopentyl (1-methylcyclopropyl)carbamate

(1R,3S)-3-(3-(2-(2-formyl-3-hydroxy-5-methoxyphenoxy)acetamido)-1H-pyrazol-5-yl)cyclopentyl (1-methylcyclopropyl)carbamate (80 mg, 0.169 mmol, 1 equiv), 2-aminoethan-1-ol (20.68 mg, 0.338 mmol, 2 equiv) and THF (2 mL) were added to a 20 mL reaction flask and stirred at rt for 1 h. The mixture was purified by reverse phase HPLC, 14 to 34% with ACN/H₂O with 10 mmol/L NH₄HCO₃ as modifier to afford (1R,3S)-3-(3-(2-(3-hydroxy-2-((E)-((2-hydroxyethyl)imino)methyl)-5-methoxyphenoxy)acetamido)-1H-pyrazol-5-yl)cyclopentyl (1-methylcyclopropyl)carbamate (22.4 mg, 24.71% yield) as a yellow solid. 1H NMR (400 MHz, DMSO) δ 14.04 (s, 1H), 12.15 (s, 1H), 10.50 (s, 1H), 8.48 (d, J=8.5 Hz, 1H), 7.36 (s, 1H), 6.30 (s, 1H), 5.73 (s, 1H), 5.53 (s, 1H), 5.12-4.78 (m, 2H), 4.67 (s, 2H), 3.68 (s, 3H), 3.58 (s, 4H), 3.03 (s, 1H), 2.43-2.41 (m, 1H), 2.04-1.92 (m, 1H), 1.91-1.79 (m, 1H), 1.68 (s, 2H), 1.54 (s, 1H), 1.22 (s, 3H), 0.58 (s, 2H), 0.46 (d, J=4.8 Hz, 2H), MS (ESI) calcd. for C₂₅H₃₃N₅O₇, 515.23 m/z, found 516.20 [M+H]⁺.

### Example 21.5: Preparation of Compound 251c: (1R,3S)-3-(3-(2-(2-((E)-(ethylimino)methyl)-3-hydroxy-5-methoxyphenoxy)acetamido)-1H-pyrazol-5-yl)cyclopentyl (1-methylcyclopropyl)carbamate

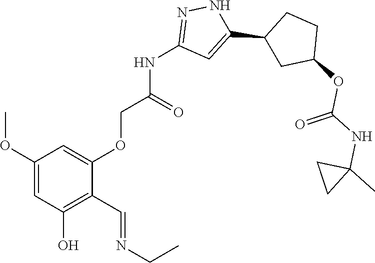 [SMILES: CC/N=C/c1c(O)cc(OC)cc1OCC(=O)Nc1cc([C@H]2CC[C@@H](OC(=O)NC3(C)CC3)C2)[nH]n1]

### Synthetic Route

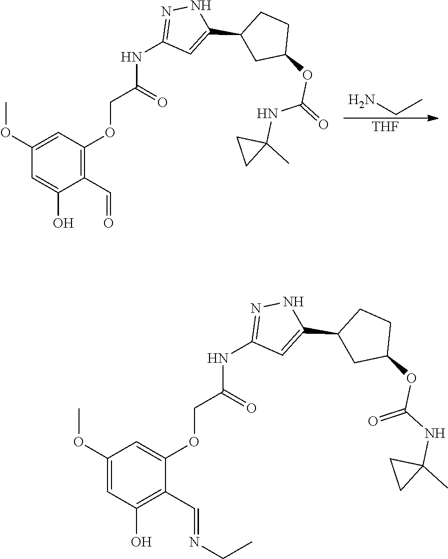

### Step 1: Synthesis of (1R,3S)-3-[5-(2-{2-[(1E)-(ethylimino)methyl]-3-hydroxy-5-methoxyphenoxy}acetamido)-2H-pyrazol-3-yl]cyclopentyl N-(1-methylcyclopropyl)carbamate

(1R,3S)-3-(3-(2-(2-formyl-3-hydroxy-5-methoxyphenoxy)acetamido)-1H-pyrazol-5-yl)cyclopentyl (1-methylcyclopropyl)carbamate (80 mg, 0.169 mmol, 1 equiv), ethanamine (15.27 mg, 0.338 mmol, 2 equiv) and THF (2 mL) were added to a 20 mL reaction flask and stirred at rt for 1 h. The resulting mixture was purified by reverse phase HPLC, 19 to 39% with ACN/H₂O (10 mmol/L NH₄HCO₃ as modifier) to afford (1R,3S)-3-(3-(2-(2-((E)-(ethylimino)methyl)-3-hydroxy-5-methoxyphenoxy)acetamido)-1H-pyrazol-5-yl)cyclopentyl (1-methylcyclopropyl)carbamate (15.6 mg, 17.52% yield) as a light yellow solid. 1H NMR (400 MHz, DMSO) δ 14.06 (s, 1H), 12.16 (s, 1H), 10.50 (s, 1H), 8.56 (d, J=8.6 Hz, 1H), 7.36 (s, 1H), 6.30 (s, 1H), 5.77 (s, 1H), 5.59 (s, 1H), 4.97 (s, 1H), 4.68 (s, 2H), 3.68 (s, 3H), 3.58 (s, 2H), 3.03 (s, 1H), 2.43-2.41 (m, 1H), 1.98 (s, 1H), 1.87 (s, 1H), 1.67 (s, 2H), 1.53-1.45 (m, 1H), 1.28-1.20 (m, 6H), 0.58 (s, 2H), 0.46 (d, J=4.6 Hz, 2H), MS (ESI) calcd. for C₂₅H₃₃N₅O₆, 499.24 m/z, found [M+H]⁺ 500.35 m/z.

### Example 21.6: Preparation of Compound 64d: (1R,3S)-3-(3-(2-(2-((E)-(butylimino)methyl)-3-hydroxy-5-methoxyphenoxy)acetamido)-1H-pyrazol-5-yl)cyclopentyl isopropylcarbamate

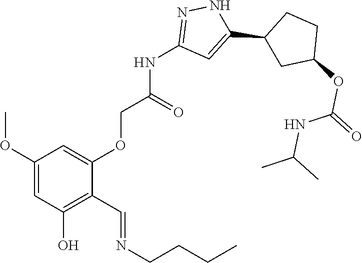 [SMILES: CCCC/N=C/c1c(O)cc(OC)cc1OCC(=O)Nc1cc([C@H]2CC[C@@H](OC(=O)NC(C)C)C2)[nH]n1]

### Synthetic Route

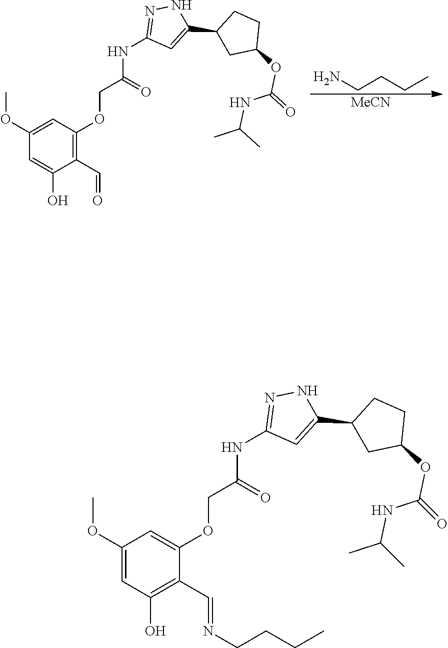

### Step 1: Synthesis of (1R,3S)-3-(3-(2-(2-((E)-(butylimino)methyl)-3-hydroxy-5-methoxyphenoxy)acetamido)-1H-pyrazol-5-yl)cyclopentyl isopropylcarbamate

(1R,3S)-3-(3-(2-(2-formyl-3-hydroxy-5-methoxyphenoxy)acetamido)-1H-pyrazol-5-yl)cyclopentyl isopropylcarbamate (90 mg, 0.195 mmol, 1 equiv.), butan-1-amine (42.9 mg, 0.585 mmol, 3 equiv.), acetonitrile (2 mL) and a stir bar were added to an oven dried 20 mL vial and stirred until homogenous. The resulting mixture was stirred at room temperature for 2 h, then purified by reverse-phase chromatography (5 to 50% ACN/H₂O to afford (1R,3S)-3-(3-(2-(2-((E)-(butylimino)methyl)-3-hydroxy-5-methoxyphenoxy)acetamido)-1H-pyrazol-5-yl)cyclopentyl isopropylcarbamate (18.4 mg, 0.035 mmol, 18% yield). ¹H NMR (300 MHz, DMSO-d6) δ 14.41-14.27 (m, 1H), 12.14 (s, 1H), 10.46 (s, 1H), 8.54 (d, J=7.6 Hz, 1H), 6.92 (d, J=7.7 Hz, 1H), 6.30 (s, 1H), 5.76 (d, J=2.1 Hz, 1H), 5.58 (d, J=2.2 Hz, 1H), 5.06-4.92 (m, 1H), 4.67 (s, 2H), 3.68 (s, 3H), 3.64-3.46 (m, 3H), 3.13-2.98 (m, 1H), 2.46-2.37 (m, 1H), 2.12-1.80 (m, 2H), 1.79-1.66 (m, 2H), 1.65-1.44 (m, 3H), 1.44-1.27 (m, 2H), 1.02 (d, J=6.5 Hz, 6H), 0.92 (t, J=7.3 Hz, 3H). MS (ESI) calcd. for C₂₆H₃₇N₅O₆, 515.27 m/z, found [M+H]⁺ 516.20 m/z.

### Example 21.7: Preparation of Compound 64e: ((((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)methyl)phosphonic acid

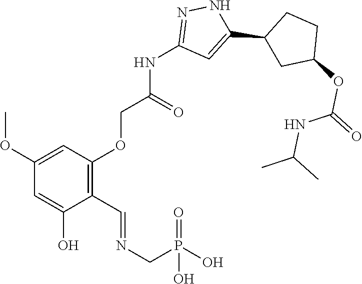 [SMILES: COc1cc(O)c(/C=N/CP(=O)(O)O)c(OCC(=O)Nc2cc([C@H]3CC[C@@H](OC(=O)NC(C)C)C3)[nH]n2)c1]

### Synthetic Route

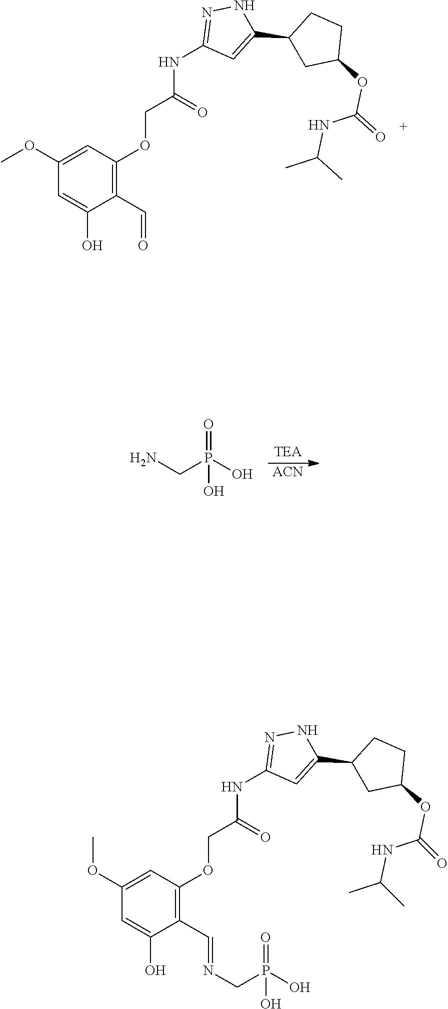

### Step 1: Synthesis of ((((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)methyl)phosphonic acid

(1R,3S)-3-(3-(2-(2-formyl-3-hydroxy-5-methoxyphenoxy)acetamido)-1H-pyrazol-5-yl)cyclopentyl isopropylcarbamate (100 mg, 0.217 mmol, 1 equiv.), (aminomethyl)phosphonic acid (72.3 mg, 0.651 mmol, 3 equiv.), acetonitrile (4 mL) and a stir bar were added to an oven dried 20 mL vial and stirred until homogenous. The resulting mixture was stirred overnight at 40° C., then purified by reverse phase preparatory HPLC, 0 to 20% ACN/H₂O to afford ((((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)methyl)phosphonic acid (15.6 mg, 0.028 mmol, 11.46% yield). ¹H NMR (400 MHz, DMSO-d6) 10.52 (s, 1H), 8.59 (s, 1H), 6.95 (s, 1H), 6.30 (s, 1H), 5.81 (s, 1H), 5.63 (s, 1H), 5.06-4.92 (s, 1H), 4.68 (s, 2H), 3.68 (s, 5H), 3.61-3.49 (m, 2H), 3.12-2.95 (m, 2H), 2.10-1.93 (m, 1H), 1.92-1.80 (m, 3H), 1.78-1.66 (m, 2H), 1.02 (d, J=6.5 Hz, 6H). MS (ESI) mass calcd. for C₂₃H₃₂N₅O₉P 553.19; m/z, found [M+H]+ 554.15 m/z.

### Example 21.8: Preparation of Compound 145a: (1R,3S)-3-(3-(2-(3-hydroxy-2-((E)-(isopropylimino)methyl)-5-methylphenoxy)acetamido)-1H-pyrazol-5-yl)cyclopentyl (1-methylcyclopropyl)carbamate

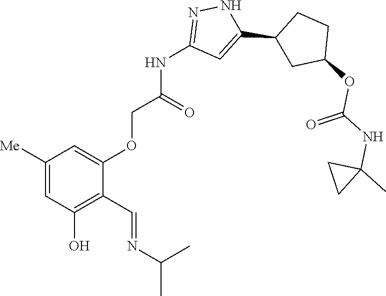 [SMILES: Cc1cc(O)c(/C=N/C(C)C)c(OCC(=O)Nc2cc([C@H]3CC[C@@H](OC(=O)NC4(C)CC4)C3)[nH]n2)c1]

### Synthetic Route

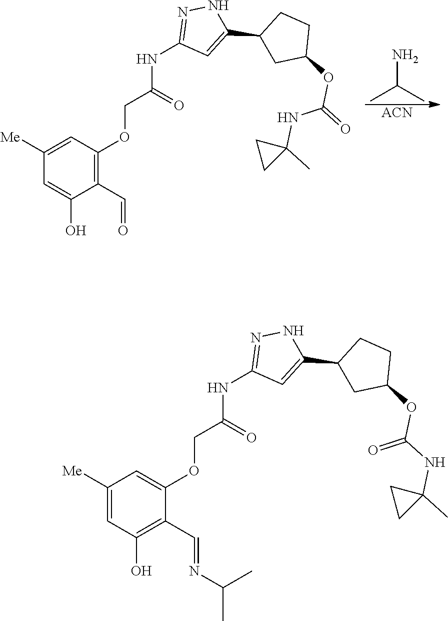

### Step 1: Synthesis of (1R,3S)-3-(3-(2-(3-hydroxy-2-((E)-(isopropylimino)methyl)-5-methylphenoxy)acetamido)-1H-pyrazol-5-yl)cyclopentyl (1-methylcyclopropyl)carbamate

A suspension of (1R,3S)-3-(3-(2-(2-formyl-3-hydroxy-5-methylphenoxy)acetamido)-1H-pyrazol-5-yl)cyclopentyl (1-methylcyclopropyl)carbamate (50 mg, 0.110 mmol, 1 equiv) and propan-2-amine (19.42 mg, 0.330 mmol, 3 equiv) in ACN (2 mL) was stirred at rt for 1 h. The resulting mixture was purified by reverse phase HPLC, 0 to 50% with ACN/H₂O to afford (1R,3S)-3-(3-(2-(3-hydroxy-2-((E)-(isopropylimino)methyl)-5-methylphenoxy)acetamido)-1H-pyrazol-5-yl)cyclopentyl (1-methylcyclopropyl)carbamate (16.5 mg, 27.8% yield) as a yellow solid. 1H NMR (400 MHz, DMSO) δ 14.58 (s, 1H), 12.14 (s, 1H), 10.46 (s, 1H), 8.83 (d, J=2.1 Hz, 1H), 7.35 (s, 1H), 6.30 (s, 1H), 6.25 (s, 1H), 6.11 (s, 1H), 4.97 (s, 1H), 4.71 (s, 2H), 3.69-3.59 (m, 1H), 3.03 (s, 1H), 2.48-2.40 (m, 1H), 2.19 (s, 3H), 1.99 (d, J=9.9 Hz, 1H), 1.87 (s, 1H), 1.68 (s, 2H), 1.54 (s, 1H), 1.43-1.41 (m, 3H), 1.24 (d, J=6.4 Hz, 6H), 0.58 (s, 2H), 0.45-0.43 (m, 2H), MS (ESI) calcd. for C₂₆H₃₅N₅O₅, 497.20 m/z, found [M+H]⁺ 498.15 m/z.

### Example 21.9: Preparation of Compound 64f (1R,3S)-3-(3-(2-(3-hydroxy-5-methoxy-2-((E)-(phenethylimino)methyl)phenoxy)acetamido)-1H-pyrazol-5-yl)cyclopentyl isopropylcarbamate

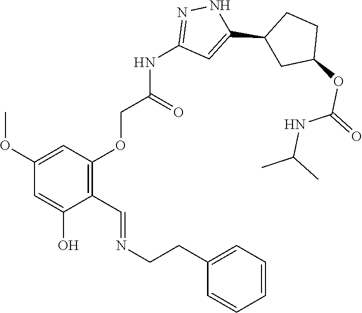 [SMILES: COc1cc(O)c(/C=N/CCc2ccccc2)c(OCC(=O)Nc2cc([C@H]3CC[C@@H](OC(=O)NC(C)C)C3)[nH]n2)c1]

### Synthetic Route

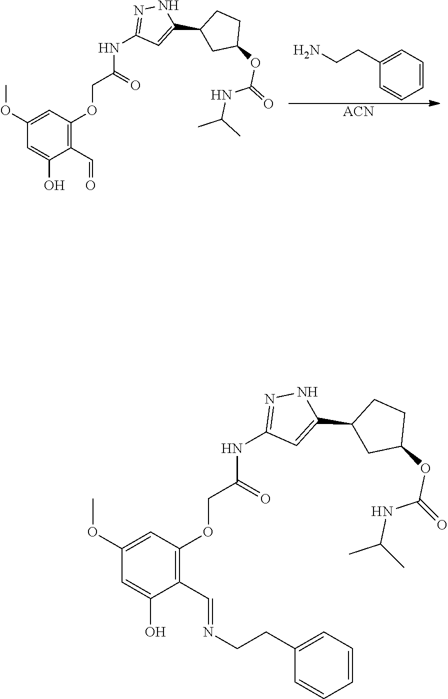 [SMILES: COc1cc(O)c(C=O)c(OCC(=O)Nc2cc([C@H]3CC[C@@H](OC(=O)NC(C)C)C3)[nH]n2)c1 ; NCCc1ccccc1 ; "ACN" ; COc1cc(O)c(/C=N/CCc2ccccc2)c(OCC(=O)Nc2cc([C@H]3CC[C@@H](OC(=O)NC(C)C)C3)[nH]n2)c1]

### Step 1: Synthesis of (1R,3S)-3-(3-(2-(3-hydroxy-5-methoxy-2-((E)-(phenethylimino)methyl)phenoxy)acetamido)-1H-pyrazol-5-yl)cyclopentyl isopropylcarbamate

(1R,3S)-3-(3-(2-(2-formyl-3-hydroxy-5-methoxyphenoxy)acetamido)-1H-pyrazol-5-yl)cyclopentyl isopropylcarbamate (100 mg, 0.217 mmol, 1 equiv), 2-phenylethan-1-amine (52.63 mg, 0.434 mmol, 2 equiv) and ACN (1 mL) were added to a 20-mL reaction flask and stirred at rt for 1 h. The resulting mixture was purified by reverse phase HPLC, 50 to 55% with ACN/H₂O to afford (1R,3S)-3-(3-(2-(3-hydroxy-5-methoxy-2-((E)-(phenethylimino)methyl)phenoxy)acetamido)-1H-pyrazol-5-yl)cyclopentyl isopropylcarbamate (22.3 mg, 17.8% yield) as a yellow solid. 1H NMR (300 MHz, DMSO) δ 14.32-14.23 (m, 1H), 12.15 (s, 1H), 10.47 (s, 1H), 8.52 (d, J=7.2 Hz, 1H), 7.37-7.23 (m, 5H), 6.94 (d, J=7.7 Hz, 1H), 6.31 (s, 1H), 5.78 (d, J=2.1 Hz, 1H), 5.60 (d, J=2.1 Hz, 1H), 4.99 (s, 1H), 4.66 (s, 2H), 3.80 (s, 2H), 3.68 (s, 3H), 3.57-3.46 (m, 1H), 3.11-2.99 (m, 1H), 2.99-2.87 (m, 2H), 2.05-1.83 (m, 1H), 1.71 (d, J=7.9 Hz, 2H), 1.78-1.45 (m, 3H), 1.02 (d, J=6.5 Hz, 6H), MS (ESI) calcd. for C₃₀H₃₇N₅O₆, 563.27 m/z, found [M+H]⁺ 564.20 m/z.

### Example 21.10: Preparation of Compound 64g: (1R,3S)-3-(3-(2-(2-((E)-(benzylimino)methyl)-3-hydroxy-5-methoxyphenoxy)acetamido)-1H-pyrazol-5-yl)cyclopentyl isopropylcarbamate

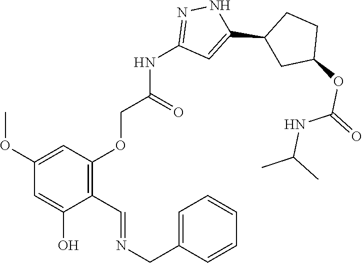 [SMILES: COc1cc(O)c(/C=N/Cc2ccccc2)c(OCC(=O)Nc2cc([C@H]3CC[C@@H](OC(=O)NC(C)C)C3)[nH]n2)c1]

### Synthetic Route

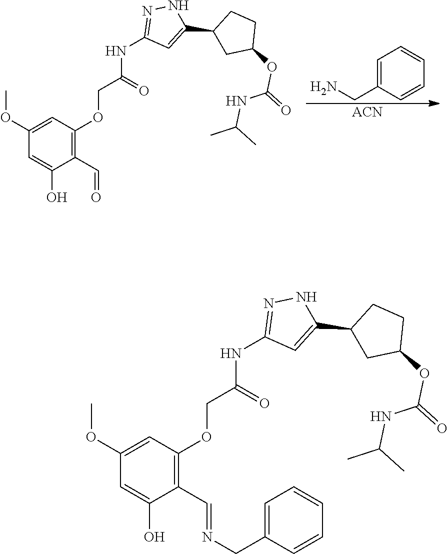

### Step 1: Synthesis of (1R,3S)-3-(3-(2-(2-((E)-(benzylimino)methyl)-3-hydroxy-5-methoxyphenoxy)acetamido)-1H-pyrazol-5-yl)cyclopentyl isopropylcarbamate

A suspension of (1R,3S)-3-(3-(2-(2-formyl-3-hydroxy-5-methoxyphenoxy)acetamido)-1H-pyrazol-5-yl)cyclopentyl isopropylcarbamate (100 mg, 0.217 mmol, 1 equiv) and benzylamine (34.91 mg, 0.326 mmol, 1.5 equiv) in ACN (5 mL) was stirred at 22° C. for 2 h. The mixture was purified by reversed-phase flash chromatography from 30 to 50% with ACN/H₂O to afford (1R,3S)-3-(3-(2-(2-((E)-(benzylimino)methyl)-3-hydroxy-5-methoxyphenoxy)acetamido)-1H-pyrazol-5-yl)cyclopentyl isopropylcarbamate (38.9 mg, 31.34% yield) as a yellow solid. 1H NMR (300 MHz, DMSO) δ 14.49 (s, 1H), 12.17 (s, 1H), 10.54 (s, 1H), 8.77 (d, J=6.4 Hz, 1H), 7.49-7.25 (m, 5H), 6.96 (d, J=7.8 Hz, 1H), 6.31 (s, 1H), 5.84-5.69 (m, 2H), 4.99 (s, 1H), 4.79-4.72 (m, 4H), 3.70 (s, 3H), 3.60-3.52 (m, 1H), 3.10-2.99 (m, 1H), 2.47-2.40 (m, 1H), 2.07-1.79 (m, 2H), 1.75-1.56 (m, 3H), 1.02 (d, J=6.5 Hz, 6H), MS (ESI) calcd. for C₂₉H₃₅N₅O₆, 549.26 m/z, found [M+H]⁺ 550.15 m/z.

### Example 21.11: Preparation of Compound 64h: (1R,3S)-3-(3-(2-(2-((E)-(tert-butylimino)methyl)-3-hydroxy-5-methoxyphenoxy)acetamido)-1H-pyrazol-5-yl)cyclopentyl isopropylcarbamate

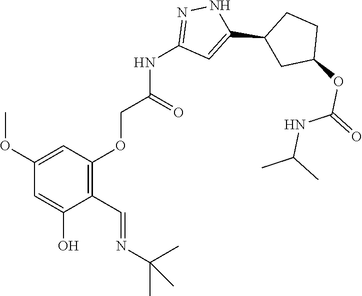 [SMILES: COc1cc(O)c(/C=N/C(C)(C)C)c(OCC(=O)Nc2cc([C@H]3CC[C@@H](OC(=O)NC(C)C)C3)[nH]n2)c1]

### Synthetic Route

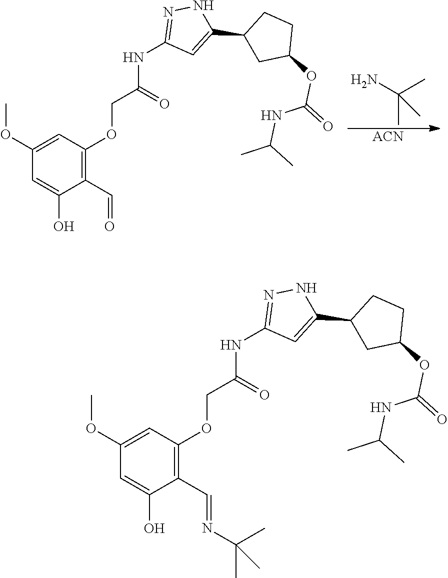

### Step 1: Synthesis of (1R,3S)-3-(3-(2-(2-((E)-(tert-butylimino)methyl)-3-hydroxy-5-methoxyphenoxy)acetamido)-1H-pyrazol-5-yl)cyclopentyl isopropylcarbamate

A suspension of (1R,3S)-3-(3-(2-(2-formyl-3-hydroxy-5-methoxyphenoxy)acetamido)-1H-pyrazol-5-yl)cyclopentyl isopropylcarbamate (100 mg, 0.217 mmol, 1 equiv) and 2-methylpropan-2-amine (23.82 mg, 0.326 mmol, 1.5 equiv) in ACN (5 mL) was stirred at 22° C. for 2 h. The resulting mixture was purified by reverse column from 30 to 50% with ACN/H₂O to afford (1R,3S)-3-(3-(2-(2-((E)-(tert-butylimino)methyl)-3-hydroxy-5-methoxyphenoxy)acetamido)-1H-pyrazol-5-yl)cyclopentyl isopropylcarbamate (55.9 mg, 49.14% yield) as a yellow solid. 1H NMR (300 MHz, DMSO) δ 14.90 (d, J=9.2 Hz, 1H), 12.16 (s, 1H), 10.50 (s, 1H), 8.53 (d, J=9.2 Hz, 1H), 6.97 (d, J=7.8 Hz, 1H), 6.32 (s, 1H), 5.71 (d, J=2.1 Hz, 1H), 5.53 (d, J=2.1 Hz, 1H), 4.99 (s, 1H), 4.68 (s, 2H), 3.68 (s, 3H), 3.60-3.72 (m, 1H), 3.12-2.98 (m, 1H), 2.47-2.40 (m, 1H), 2.07-1.79 (m, 2H), 1.78-1.50 (m, 3H), 1.36 (s, 9H), 1.09-1.01 (m, 6H), MS (ESI) calcd. for C₂₆H₃₇N₅O₆, 515.20 m/z, found 516.20 [M+H]⁺.

### Example 21.12: Preparation of Compound 64i: (1R,3S)-3-(3-(2-(2-((E)-(cyclohexylimino)methyl)-3-hydroxy-5-methoxyphenoxy)acetamido)-1H-pyrazol-5-yl)cyclopentyl isopropylcarbamate

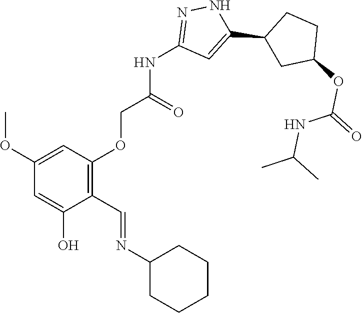 [SMILES: COc1cc(O)c(/C=N/C2CCCCC2)c(OCC(=O)Nc2cc([C@H]3CC[C@@H](OC(=O)NC(C)C)C3)[nH]n2)c1]

### Synthetic Route

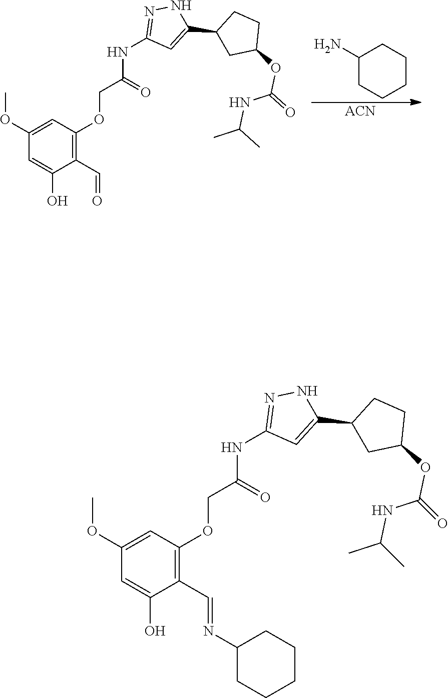

### Step 1: Synthesis of (1R,3S)-3-(3-(2-(2-((E)-(cyclohexylimino)methyl)-3-hydroxy-5-methoxyphenoxy)acetamido)-1H-pyrazol-5-yl)cyclopentyl isopropylcarbamate

(1R,3S)-3-(5-[2-(2-formyl-3-hydroxy-5-methoxyphenoxy)acetamido]-2H-pyrazol-3-ylcyclopentyl N-isopropylcarbamate (100 mg, 0.217 mmol, 1 equiv.), a stir bar, cyclohexylamine (65 mg, 0.655 mmol, 3 equiv.) and ACN (4 mL) were added to a 20 mL vial and stirred until homogenous. The resulting mixture was stirred for 1 h at rt. The crude was purified by reverse-phase chromatography, 0 to 40% ACN/H₂O to afford (1R,3S)-3-(3-(2-(2-((E)-(cyclohexylimino)methyl)-3-hydroxy-5-methoxyphenoxy)acetamido)-1H-pyrazol-5-yl)cyclopentyl isopropylcarbamate (11.2 mg, 9.35% yield) as a yellow solid. MS (ESI) mass calcd. for C₂₈H₃₉N₅O₆, 541.29; m/z found, 542.45 [M+H]⁺. 1H NMR (400 MHz, DMSO-d6) δ 14.58-14.52 (m, 1H), 12.16 (s, 1H), 10.49 (s, 1H), 8.59 (d, J=7.4 Hz, 1H), 6.95 (d, J=7.8 Hz, 1H), 6.31 (s, 1H), 5.76 (d, J=2.1 Hz, 1H), 5.60 (d, J=2.2 Hz, 1H), 5.01-4.96 (m, 1H), 4.67 (s, 2H), 3.68 (s, 3H), 3.61-3.52 (m, 1H), 3.48-3.42 (m, 1H), 3.09-3.00 (m, 1H), 3.48-2.42 (m, 1H), 2.03-1.95 (m, 1H), 1.92-1.83 (m, 3H), 1.74-1.67 (m, 4H), 1.62-1.55 (m, 2H), 1.49-1.33 (m, 4H), 1.32-1.23 (m, 1H), 1.02 (d, J=6.6 Hz, 6H).

### Example 21.13: Preparation of Compound 64j: (1R,3S)-3-(3-(2-(3-hydroxy-2-(iminomethyl)-5-methoxyphenoxy)acetamido)-1H-pyrazol-5-yl)cyclopentyl isopropylcarbamate

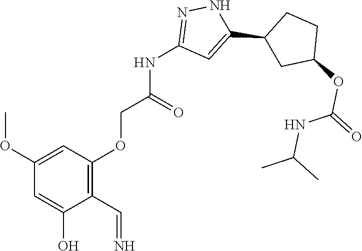 [SMILES: COc1cc(O)c(C=N)c(OCC(=O)Nc2cc([C@H]3CC[C@@H](OC(=O)NC(C)C)C3)[nH]n2)c1]

### Synthetic Route

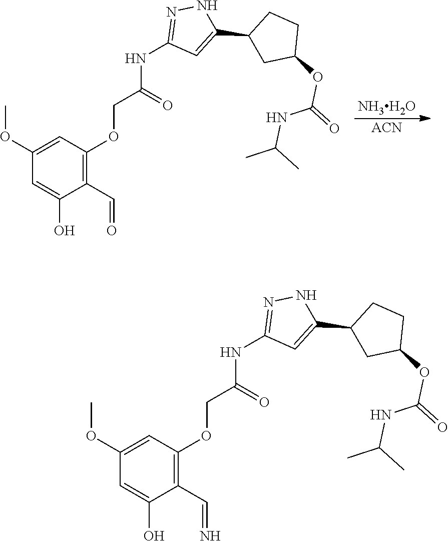

### Step 1: Synthesis of (1R,3S)-3-(3-(2-(3-hydroxy-2-(iminomethyl)-5-methoxyphenoxy)acetamido)-1H-pyrazol-5-yl)cyclopentyl isopropylcarbamate

(1R,3S)-3-(5-[2-(2-formyl-3-hydroxy-5-methoxyphenoxy)acetamido]-2H-pyrazol-3-ylcyclopentyl N-isopropylcarbamate (100 mg, 0.217 mmol, 1 equiv.), a stir bar, ACN (4 mL) and NH₃·H₂O (28%, 2.173 mmol, 10 equiv.) were added to a 20 mL vial and stirred overnight at rt. The reaction was checked by LCMS and showed complete consumption of the starting material, then concentrated to dryness in vacuo. The crude was purified by reverse phase chromatography, 0 to 38% ACN/H₂O to afford (1R,3S)-3-(3-(2-(3-hydroxy-2-(iminomethyl)-5-methoxyphenoxy)acetamido)-1H-pyrazol-5-yl)cyclopentyl isopropylcarbamate (31.0 mg, 28.97% yield) as a yellow solid. MS (ESI) calcd. for C₂₂H₂₉N₅O₆, 459.21 m/z, found, 460.30 [M+H]+. 1H NMR (400 MHz, DMSO-d6) δ 12.43-12.36 (m, 1H), 12.15 (s, 1H), 10.51 (s, 1H), 9.35 (s, 1H), 8.36 (dd, J=14.2, 8.8 Hz, 1H), 6.95 (d, J=7.8 Hz, 1H), 6.32 (s, 1H), 5.54 (d, J=2.1 Hz, 1H), 5.33 (d, J=2.1 Hz, 1H), 5.01-4.96 (m, 1H), 4.62 (s, 2H), 3.65 (s, 3H), 3.61-3.51 (m, 1H), 3.08-3.00 (m, 1H), 2.49-2.42 (m, 1H), 2.03-1.95 (m, 1H), 1.95-1.81 (m, 1H), 1.72-1.63 (m, 2H), 1.60-1.54 (m, 1H), 1.02 (d, J=6.5 Hz, 6H).

### Example 21.14: Preparation of Compound 64k: 3-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)propanoic acid

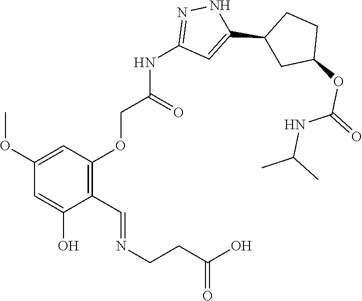 [SMILES: COc1cc(O)c(/C=N/CCC(=O)O)c(OCC(=O)Nc2cc([C@H]3CC[C@@H](OC(=O)NC(C)C)C3)[nH]n2)c1]

### Synthetic Route

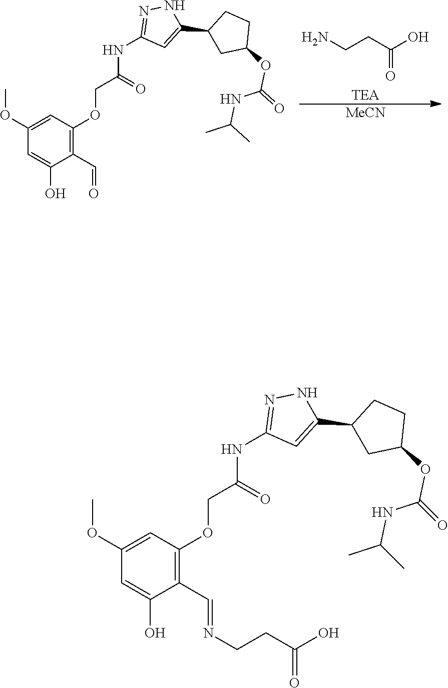

### Step 1: Synthesis of 3-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)propanoic acid

(1R,3S)-3-(3-(2-(2-formyl-3-hydroxy-5-methoxyphenoxy)acetamido)-1H-pyrazol-5-yl)cyclopentyl isopropylcarbamate (80 mg, 0.174 mmol, 1 equiv.), 3-aminopropanoic acid (40.0 mg, 0.348 mmol, 2 equiv.), acetonitrile (2 mL) and a stir bar were added to an oven dried 20 mL vial and stirred until homogenous. The resulting mixture was treated with TEA (105.5 mg, 1.044 mmol, 6 equiv.) dropwise. The resulting mixture was stirred overnight at 40° C., then cooled to room temperature and purified by reverse-phase chromatography, 0 to 21% ACN/H₂O to afford 3-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)propanoic acid (38.1 mg, 0.072 mmol, 38.6% yield). 1H NMR (400 MHz, DMSO-d6) δ 14.07 (s, 1H), 12.14 (s, 1H), 10.49 (s, 1H), 8.58 (s, 1H), 6.95 (d, J=7.8 Hz, 1H), 6.30 (s, 1H), 5.77 (d, J=2.1 Hz, 1H), 5.60 (d, J=2.2 Hz, 1H), 5.03-4.94 (m, 1H), 4.68 (s, 2H), 3.76-3.65 (m, 5H), 3.62-3.51 (m, 1H), 3.09-2.99 (m, 1H), 2.59 (t, J=6.34 Hz, 2H), 2.48-2.41 (m, 1H), 2.06-1.94 (m, 1H), 1.94-1.79 (m, 1H), 1.78-1.64 (m, 2H), 1.63-1.52 (m, 1H), 1.05-1.00 (m, 6H), 1.00-0.96 (m, 1H). MS (ESI) mass calcd. for C₂₅H₃₃N₅O₈ 531.23 m/z, found [M+H]+ 532.35.

### Example 21.15: Preparation of Compound 64l: (1R,3S)-3-(3-(3-(3-hydroxy-5-methoxy-2-((E)-((2-methoxyethyl)imino)methyl)phenyl)propanamido)-1H-pyrazol-5-yl)cyclopentyl isopropylcarbamate

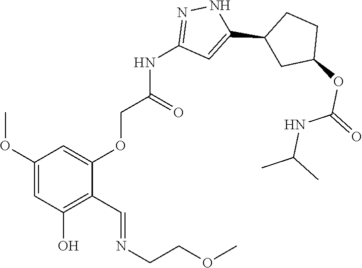 [SMILES: COCC/N=C/c1c(O)cc(OC)cc1OCC(=O)Nc1cc([C@H]2CC[C@@H](OC(=O)NC(C)C)C2)[nH]n1]

### Synthetic Route

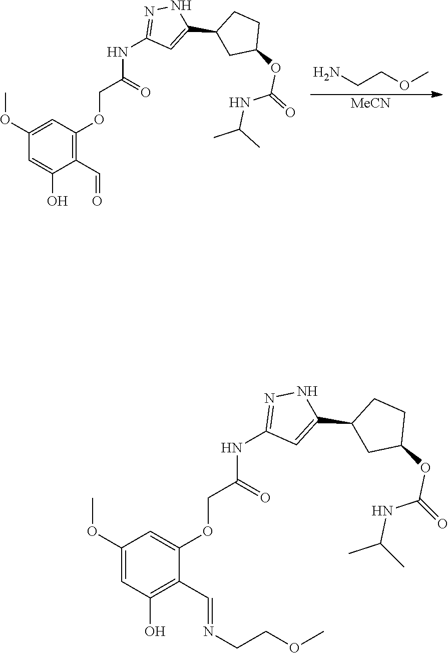

### Step 1: Synthesis of (1R,3S)-3-(3-(3-(3-hydroxy-5-methoxy-2-((E)-((2-methoxyethyl)imino)methyl)phenyl)propanamido)-1H-pyrazol-5-yl)cyclopentyl isopropylcarbamate

(1R,3S)-3-(3-(2-(2-formyl-3-hydroxy-5-methoxyphenoxy)acetamido)-1H-pyrazol-5-yl)cyclopentyl isopropylcarbamate (80 mg, 0.174 mmol, 1 equiv.), 2-methoxyethan-1-amine (65.2 mg, 0.870 mmol, 5 equiv.), acetonitrile (4 mL) and a stir bar were added to an oven dried 8 mL vial and stirred until homogenous. The resulting mixture was stirred overnight at room temperature, the reaction was checked by LCMS and showed complete consumption of starting material. The resulting mixture was purified by reverse-phase chromatography, 5 to 42% ACN/H₂O to afford (1R,3S)-3-(3-(3-(3-hydroxy-5-methoxy-2-((E)-((2-methoxyethyl)imino)methyl)phenyl)propanamido)-1H-pyrazol-5-yl)cyclopentyl isopropylcarbamate (35.6 mg, 0.069 mmol, 38.3%). 1H NMR (400 MHz, DMSO-d6) δ 14.20-14.14 (m, 1H), 12.16 (s, 1H), 10.50 (s, 1H), 8.52 (d, J=7.9 Hz, 1H), 6.95 (d, J=7.8 Hz, 1H), 6.34 (s, 1H), 5.76 (d, J=2.1 Hz, 1H), 5.58 (d, J=2.2 Hz, 1H), 5.02-4.95 (m, 1H), 4.67 (s, 2H), 3.73-3.66 (m, 5H), 3.62-3.49 (s, 3H), 3.28 (s, 3H), 3.12-2.98 (m, 1H), 2.47-2.40 (m, 1H), 2.05-1.95 (m, 1H), 1.94-1.84 (m, 1H), 1.78-1.62 (m, 2H), 1.62-1.52 (m, 1H), 1.07-0.96 (m, 6H). MS (ESI) mass calcd. for C₂₅H₃₅N₅O₇ 517.25 m/z, found [M+H]+ 518.25 m/z.

### Example 21.16: Preparation of Compound 64b: (1R,3S)-3-(3-(2-(3-hydroxy-5-methoxy-2-((E)-(propylimino)methyl)phenoxy)acetamido)-1H-pyrazol-5-yl)cyclopentyl isopropylcarbamate

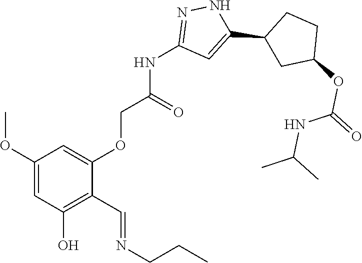 [SMILES: CCC/N=C/c1c(O)cc(OC)cc1OCC(=O)Nc1cc([C@H]2CC[C@@H](OC(=O)NC(C)C)C2)[nH]n1]

### Synthetic Route

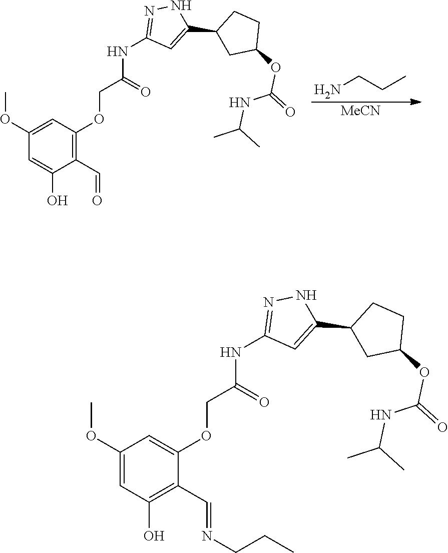

### Step 1: Synthesis of (1R,3S)-3-(3-(2-(3-hydroxy-5-methoxy-2-((E)-(propylimino)methyl)phenoxy)acetamido)-1H-pyrazol-5-yl)cyclopentyl isopropylcarbamate

(1R,3S)-3-{5-[2-(2-formyl-3-hydroxy-5-methoxyphenoxy)acetamido]-2H-pyrazol-3-yl}cyclopentyl N-isopropylcarbamate (80 mg, 0.174 mmol), a stir bar, ACN (2 mL) were added to a 8 mL vial and stirred until homogeneous, and then treated with propan-1-amine (40 mg, 0.887 mmol). The resulting mixture was stirred for 2 h at rt, then purified by reverse-phase chromatography, 0 to 50% ACN/H₂O to afford (1R,3S)-3-[5-(2-{3-hydroxy-5-methoxy-2-[(1E)-[(2-methylpropyl)imino]methyl]phenoxy}acetamido)-2H-pyrazol-3-yl]cyclopentyl N-isopropylcarbamate as a yellow solid (22.9 mg, 34.44%). MS (ESI): mass calcd. for C₂₅H₃₅N₅O₆, 501.25; m/z found, 502.25 [M+H]⁺. 1H NMR (400 MHz, DMSO-d6) δ 14.46-14.17 (m, 1H), 12.15 (s, 1H), 10.49 (s, 1H), 8.53 (d, J=7.9 Hz, 1H), 6.95 (d, J=7.8 Hz, 1H), 6.31 (s, 1H), 5.82-5.70 (m, 1H), 5.67-5.50 (m, 1H), 4.98 (s, 1H), 4.67 (s, 2H), 3.68 (s, 3H), 3.60-3.44 (m, 3H), 3.12-2.96 (m, 1H), 2.75-2.56 (m, 1H), 2.00 (d, J=8.9 Hz, 1H), 1.96-1.81 (m, 1H), 1.78-1.52 (m, 5H), 1.02 (d, J=6.5 Hz, 6H), 0.92 (t, J=7.3 Hz, 3H).

### Example 21.17: Preparation of Compound 64m: (1R,3S)-3-(3-(2-(3-hydroxy-2-((E)-(isobutylimino)methyl)-5-methoxyphenoxy)acetamido)-1H-pyrazol-5-yl)cyclopentyl isopropylcarbamate

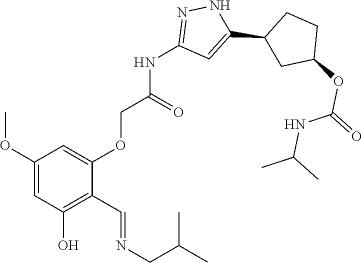 [SMILES: COc1cc(O)c(/C=N/CC(C)C)c(OCC(=O)Nc2cc([C@H]3CC[C@@H](OC(=O)NC(C)C)C3)[nH]n2)c1]

### Synthetic Route

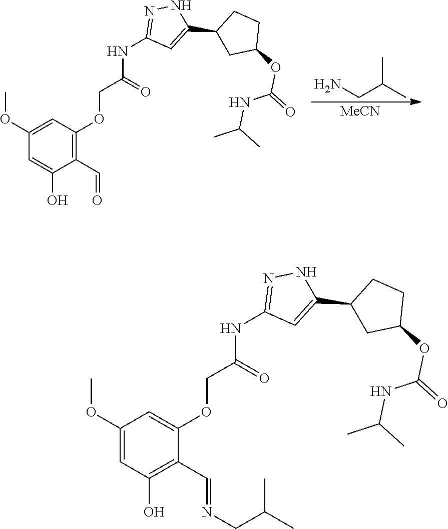

### Step 1: Synthesis of (1R,3S)-3-(3-(2-(3-hydroxy-2-((E)-(isobutylimino)methyl)-5-methoxyphenoxy)acetamido)-1H-pyrazol-5-yl)cyclopentyl isopropylcarbamate

(1R,3S)-3-{5-[2-(2-formyl-3-hydroxy-5-methoxyphenoxy)acetamido]-2H-pyrazol-3-yl}cyclopentyl N-isopropylcarbamate (80 mg, 0.174 mmol), a stir bar, ACN (2 mL) were added to a 8 mL vial and stirred until homogeneous, and then treated with 2-methylpropan-1-amine (40 mg, 0.547 mmol). The resulting mixture was stirred for 2 h at rt, then purified by reverse-phase chromatography, 0 to 50% ACN/H2O to afford (1R,3S)-3-[5-(2-{3-hydroxy-5-methoxy-2-[(1E)-[(2-methylpropyl)imino]methyl]phenoxy}acetamido)-2H-pyrazol-3-yl]cyclopentyl N-isopropylcarbamate as a yellow solid (19.6 mg, 28.53%). MS (ESI) mass calcd. for C₂₆H₃₇N₅O₆, 515.27; m/z found, 516.30 [M+H]+. 1H NMR (400 MHz, DMSO-d6) δ 14.63-14.36 (m, 1H), 12.16 (s, 1H), 10.50 (s, 1H), 8.52 (d, J=7.8 Hz, 1H), 6.95 (d, J=7.8 Hz, 1H), 6.31 (s, 1H), 5.85-5.72 (m, 1H), 5.67-5.46 (m, 1H), 4.98 (s, 1H), 4.68 (s, 2H), 3.68 (s, 3H), 3.56 (d, J=7.1 Hz, 1H), 3.46-3.36 (m, 2H), 3.12-2.94 (m, 1H), 2.07-1.93 (m, 1H), 1.96-1.79 (m, 2H), 1.81-1.65 (m, 2H), 1.65-1.47 (m, 1H), 1.02 (d, J=6.6 Hz, 6H), 0.92 (d, J=6.7 Hz, 6H).

### Example 21.18: Preparation of Compound 64n: (1R,3S)-3-(3-(2-(2-((E)-(((S)-sec-butyl)imino)methyl)-3-hydroxy-5-methoxyphenoxy)acetamido)-1H-pyrazol-5-yl)cyclopentyl isopropylcarbamate

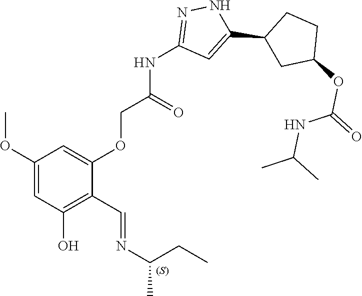

### Synthetic Route

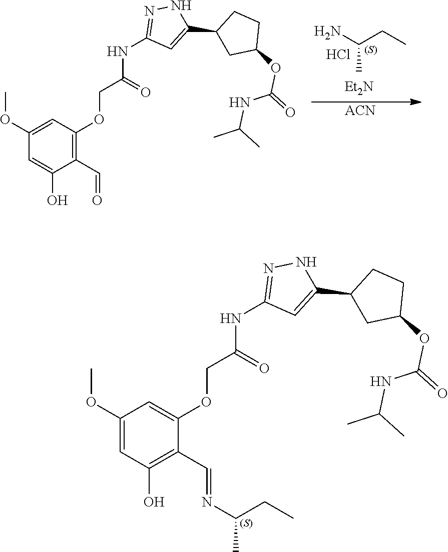

### Step 1: Synthesis of (1R,3S)-3-(3-(2-(2-((E)-(((S)-sec-butyl)imino)methyl)-3-hydroxy-5-methoxyphenoxy)acetamido)-1H-pyrazol-5-yl)cyclopentyl isopropylcarbamate

(2S)-butan-2-amine hydrochloride (72 mg, 0.657 mmol, 3.03 equiv), a stir bar, Et₃N (132 mg, 1.304 mmol, 6.01 equiv) and ACN (4 mL) were added to a 20 mL vial and stirred until homogeneous, then treated with (1R,3S)-3-(5-[2-(2-formyl-3-hydroxy-5-methoxyphenoxy)acetamido]-2H-pyrazol-3-ylcyclopentyl N-isopropylcarbamate (100 mg, 0.217 mmol, 1 equiv) in batches at rt. The resulting mixture was stirred at rt for 2 h. The resulting mixture was purified by reverse-phase chromatography, 5 to 45% MeCN/H2O to afford (1R,3S)-3-(3-(2-(2-((E)-(((S)-sec-butyl)imino)methyl)-3-hydroxy-5-methoxyphenoxy)acetamido)-1H-pyrazol-5-yl)cyclopentyl isopropylcarbamate (12.8 mg, 11.23%) as a yellow solid. 1H NMR (400 MHz, DMSO-d6) δ 14.50 (t, J=6.2 Hz, 1H), 12.16 (s, 1H), 10.50 (s, 1H), 8.57 (d, J=7.1 Hz, 1H), 6.95 (d, J=7.8 Hz, 1H), 6.31 (s, 1H), 5.79 (d, J=2.1 Hz, 1H), 5.62 (d, J=2.2 Hz, 1H), 5.02-4.95 (m, 1H), 4.68 (s, 2H), 3.68 (s, 3H), 3.61-3.44 (m, 2H), 3.08-3.00 (m, 1H), 2.48-2.40 (m, 1H), 2.04-1.97 (m, 1H), 1.95-1.83 (m, 1H), 1.74-1.45 (m, 5H), 1.24 (d, J=6.5 Hz, 3H), 1.02 (d, J=6.5 Hz, 6H), 0.87 (t, J=7.4 Hz, 3H). MS (ESI) mass calcd. for C₂₆H₃₇N₅O₆, 515.27 m/z, found, 516.35 [M+H]⁺.

### Example 21.19: Preparation of Compound 64o: (1R,3S)-3-(3-(2-(2-((E)-(((R)-sec-butyl)imino)methyl)-3-hydroxy-5-methoxyphenoxy)acetamido)-1H-pyrazol-5-yl)cyclopentyl isopropylcarbamate

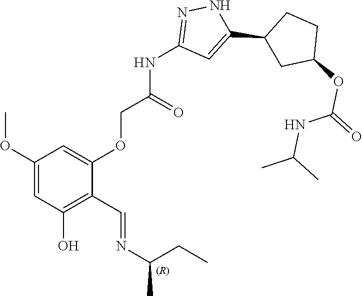

### Synthetic Route

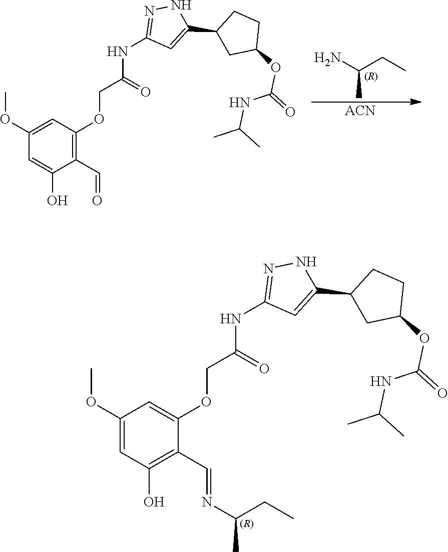

### Step 1: Synthesis of (1R,3S)-3-(3-(2-(2-((E)-(((R)-sec-butyl)imino)methyl)-3-hydroxy-5-methoxyphenoxy)acetamido)-1H-pyrazol-5-yl)cyclopentyl isopropylcarbamate

(1R,3S)-3-(5-[2-(2-formyl-3-hydroxy-5-methoxyphenoxy)acetamido]-2H-pyrazol-3-ylcyclopentyl N-isopropylcarbamate (100 mg, 0.217 mmol, 1 equiv), a stir bar, (2R)-butan-2-amine (48 mg, 0.656 mmol, 3.00 equiv) and ACN (3 mL) were added to a 20 mL vial and stirred until homogeneous. The resulting mixture was stirred at rt for 2 h. The resulting mixture was purified by reverse-phase chromatography, 5 to 45% MeCN/H₂O to afford (1R,3S)-3-(3-(2-(2-((E)-(((R)-sec-butyl)imino)methyl)-3-hydroxy-5-methoxyphenoxy)acetamido)-1H-pyrazol-5-yl)cyclopentyl isopropylcarbamate (28.4 mg, 25.17%) as a yellow solid. 1H NMR (400 MHz, DMSO-d6) δ 14.50 (t, J=6.1 Hz, 1H), 12.16 (s, 1H), 10.50 (s, 1H), 8.57 (d, J=6.7 Hz, 1H), 6.95 (d, J=7.8 Hz, 1H), 6.31 (s, 1H), 5.79 (d, J=2.1 Hz, 1H), 5.62 (d, J=2.2 Hz, 1H), 5.01-4.96 (m, 1H), 4.68 (s, 2H), 3.68 (s, 3H), 3.61-3.51 (m, 1H), 3.51-3.44 (m, 1H), 3.09-3.00 (m, 1H), 2.50-2.42 (m, 1H), 2.03-1.96 (m, 1H), 1.93-1.83 (m, 1H), 1.75-1.66 (m, 2H), 1.62-1.45 (m, 3H), 1.24 (d, J=6.4 Hz, 3H), 1.02 (d, J=6.6 Hz, 6H), 0.87 (t, J=7.4 Hz, 3H). MS (ESI) mass calcd. for C₂₆H₃₇N₅O₆, 515.27 m/z, found, 516.35 [M+H]+.

### Example 21.20: Preparation of Compound 128a: (1R,3S)-3-(3-(2-(3-hydroxy-2-((E)-(isopropylimino)methyl)-5-methoxyphenoxy)acetamido)-1H-pyrazol-5-yl)cyclopentyl tert-butylcarbamate

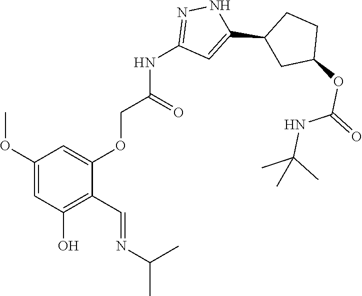 [SMILES: COc1cc(O)c(/C=N/C(C)C)c(OCC(=O)Nc2cc([C@H]3CC[C@@H](OC(=O)NC(C)(C)C)C3)[nH]n2)c1]

### Synthetic Route

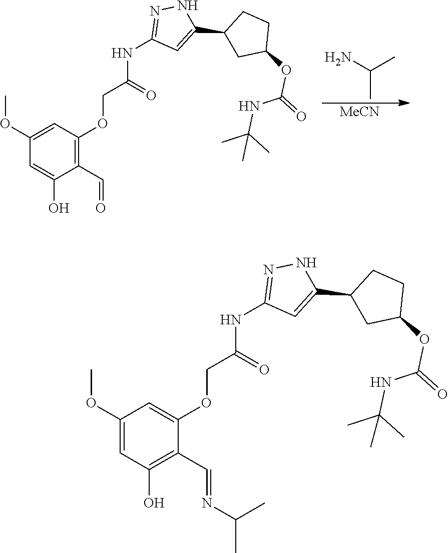

### Step 1: Synthesis of (1R,3S)-3-(3-(2-(3-hydroxy-2-((E)-(isopropylimino)methyl)-5-methoxyphenoxy)acetamido)-1H-pyrazol-5-yl)cyclopentyl tert-butylcarbamate

(1R,3S)-3-(3-(2-(2-formyl-3-hydroxy-5-methoxyphenoxy)acetamido)-1H-pyrazol-5-yl)cyclopentyl tert-butylcarbamate (82 mg, 0.173 mmol), a stir bar, ACN (2 mL) were added to a 8 mL vial and stirred until homogeneous, and then treated with propan-2-amine (52 mg, 0.880 mmol). The resulting mixture was stirred for 2 h at rt, then purified by reverse-phase chromatography, 0 to 40% ACN/H₂O to afford (1R,3S)-3-(3-(2-(3-hydroxy-2-((E)-(isopropylimino)methyl)-5-methoxyphenoxy)acetamido)-1H-pyrazol-5-yl)cyclopentyl tert-butylcarbamate as a yellow solid (30.6 mg, 34.18%). MS (ESI) mass calcd. for C₂₆H₃₇N₅O₆, 515.27 m/z found, 516.40 [M+H]⁺. 1H NMR (400 MHz, DMSO-d6) δ 14.49-14.41 (m, 1H), 12.15 (s, 1H), 10.49 (s, 1H), 8.59 (d, J=7.2 Hz, 1H), 6.76 (s, 1H), 6.31 (s, 1H), 5.80-5.75 (m, 1H), 5.63-5.58 (m, 1H), 4.96 (s, 1H), 4.68 (s, 2H), 3.83-3.62 (m, 4H), 3.08-2.99 (m, 1H), 2.47-2.40 (m, 1H), 2.02-1.96 (m, 1H), 1.87 (s, 1H), 1.74-1.63 (m, 2H), 1.56 (s, 1H), 1.26 (d, J=6.4 Hz, 6H), 1.19 (s, 9H).

### Example 21.21: Preparation of Compound 254a: (1R,3S)-3-(3-(2-(3-hydroxy-2-((E)-(isopropylimino)methyl)-5-methoxyphenoxy)acetamido)-1H-pyrazol-5-yl)cyclopentyl (1-methylcyclobutyl)carbamate

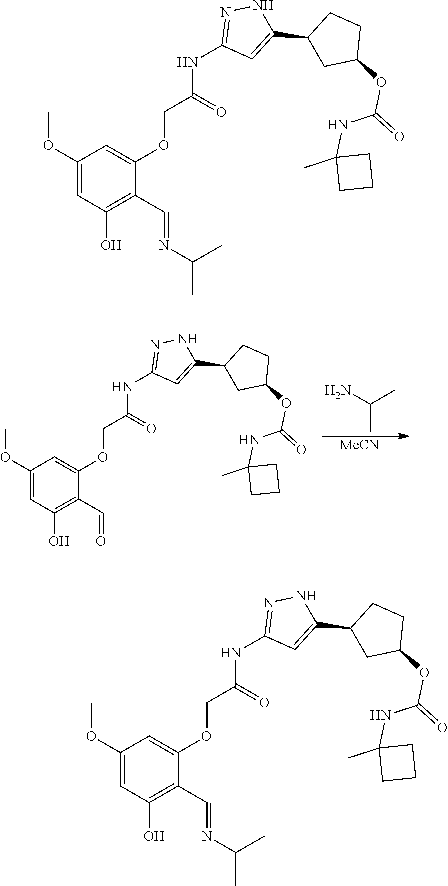

### Step 1: Synthesis of (1R,3S)-3-(3-(2-(3-hydroxy-2-((E)-(isopropylimino)methyl)-5-methoxyphenoxy)acetamido)-1H-pyrazol-5-yl)cyclopentyl (1-methylcyclobutyl)carbamate

To a stirred solution of (1R,3S)-3-{5-[2-(2-formyl-3-hydroxy-5-methoxyphenoxy)acetamido]-2H-pyrazol-3-yl}cyclopentyl N-(1-methylcyclobutyl)carbamate (70 mg, 0.144 mmol, 1 equiv) in anhydrous MeCN (5 mL) was added isopropylamine (42.52 mg, 0.720 mmol, 5 equiv) at room temperature. The reaction mixture was stirred at room temperature for 1 h. After completion of reaction, the resulting mixture was concentrated under reduced pressure. The residue was purified by C18 reverse phase chromatography, 10 to 70% MeCN/H₂O to afford (1R,3S)-3-(3-(2-(3-hydroxy-2-((E)-(isopropylimino)methyl)-5-methoxyphenoxy)acetamido)-1H-pyrazol-5-yl)cyclopentyl (1-methylcyclobutyl)carbamate (29.4 mg, 38.65% yield). 1H NMR (400 MHz, DMSO-d6) δ 14.48-14.45 (m, 1H), 12.15 (s, 1H), 10.47 (s, 1H), 8.61-8.59 (m, 1H), 7.15 (br s, 1H), 6.33 (s, 1H), 5.80 (s, 1H), 5.60 (s, 1H), 5.03-4.94 (m, 1H), 4.67 (s, 2H), 3.78-3.66 (m, 4H), 3.09-3.04 (m, 1H), 2.48-2.35 (m, 1H), 2.28-2.16 (m, 2H), 2.04-1.96 (m, 1H), 1.92-1.81 (m, 1H), 1.85-1.67 (m, 6H), 1.61-1.55 (m, 1H), 1.36-1.22 (m, 9H). MS (ESI) calcd. for C₂₇H₃₇N₅O₆ 527.27 m/z, found [M+H]⁺ 528.40 m/z.

### Example 21.22: Preparation of Compound 64c: (1R,3S)-3-(3-(2-(2-((E)-(ethylimino)methyl)-3-hydroxy-5-methoxyphenoxy)acetamido)-1H-pyrazol-5-yl)cyclopentyl isopropylcarbamate

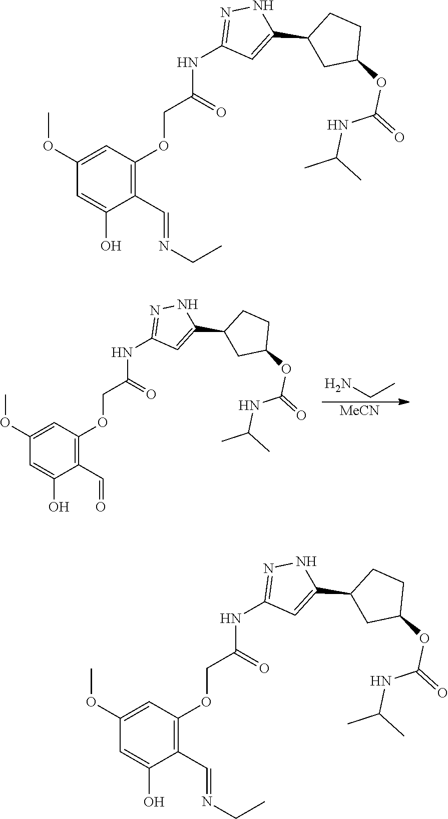

### Step 1: (1R,3S)-3-(3-(2-(2-((E)-(ethylimino)methyl)-3-hydroxy-5-methoxyphenoxy)acetamido)-1H-pyrazol-5-yl)cyclopentyl isopropylcarbamate

(1R,3S)-3-(5-[2-(2-formyl-3-hydroxy-5-methoxyphenoxy)acetamido]-2H-pyrazol-3-ylcyclopentyl N-isopropylcarbamate (80 mg, 0.174 mmol), a stir bar, ACN (2 mL) were added to a 8 mL vial and stirred until homogeneous, and then treated with ethanamine (40 mg, 0.887 mmol). The resulting mixture was stirred for 2 h at rt, then purified by reverse-phase chromatography, 0 to 50% ACN/H₂O to afford ((1R,3S)-3-[5-(2-(2-[(1E)-(ethylimino)methyl]-3-hydroxy-5-methoxyphenoxyacetamido)-2H-pyrazol-3-yl]cyclopentyl N-isopropylcarbamate as a yellow solid (15.0 mg, 17.26%). MS (ESI) mass calcd. for C₂₄H₃₃N₅O₆, 487.24 m/z, found, 488.35 [M+H]+ 0.1H NMR (400 MHz, DMSO-d6) δ14.21 (s, 1H), 12.15 (s, 1H), 10.49 (s, 1H), 8.54 (d, J=8.0 Hz, 1H), 6.95 (d, J=7.9 Hz, 1H), 6.31 (s, 1H), 5.75 (s, 1H), 5.56 (s, 1H), 4.98 (s, 1H), 4.67 (s, 2H), 3.68 (s, 3H), 3.64-3.49 (m, 3H), 3.04 (t, J=9.1 Hz, 1H), 2.47-2.40 (m, 1H), 2.06-1.96 (m, 1H), 1.95-1.80 (m, 1H), 1.77-1.62 (m, 1H), 1.65-1.53 (m, 1H), 1.23 (t, J=7.2 Hz, 3H), 1.02 (d, J=6.6 Hz, 6H).

### Example 21.23: Preparation of Compound 64p: Ethyl 2-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)acetate

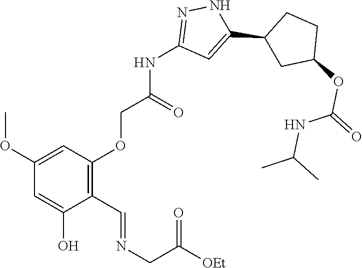 [SMILES: CCOC(=O)C/N=C/c1c(O)cc(OC)cc1OCC(=O)Nc1cc([C@H]2CC[C@@H](OC(=O)NC(C)C)C2)[nH]n1]

### Synthetic Route

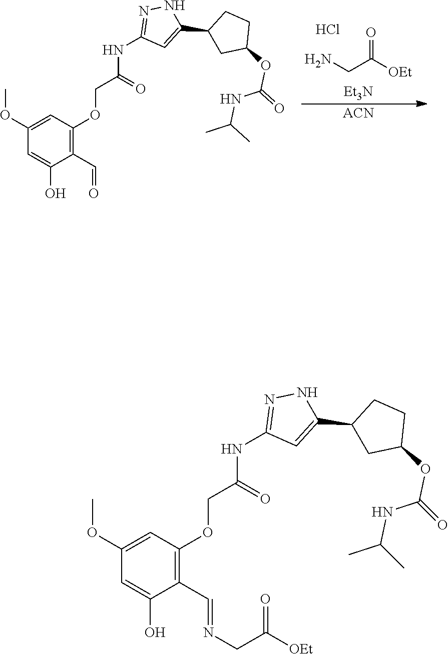 [SMILES: "Et3N" ; COc1cc(O)c(C=O)c(OCC(=O)Nc2cc([C@H]3CC[C@@H](OC(=O)NC(C)C)C3)[nH]n2)c1 ; Cl ; "ACN" ; CCOC(=O)CN ; CCOC(=O)C/N=C/c1c(O)cc(OC)cc1OCC(=O)Nc1cc([C@H]2CC[C@@H](OC(=O)NC(C)C)C2)[nH]n1]

### Step 1: Synthesis of Ethyl 2-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)acetate

Ethyl 2-aminoacetate hydrochloride (91 mg, 0.652 mmol, 3.00 equiv), a stir bar, Et₃N (132 mg, 1.304 mmol, 6.00 equiv) and ACN (5 mL) were added to a 20 mL vial and stirred until homogenous, then treated with (1R,3S)-3-(5-[2-(2-formyl-3-hydroxy-5-methoxyphenoxy)acetamido]-2H-pyrazol-3-ylcyclopentyl N-isopropylcarbamate (100 mg, 0.217 mmol, 1 equiv) in batches at rt. The resulting mixture was stirred at rt overnight. The resulting mixture was concentrated in vacuo and purified by reverse-phase chromatography, 5-40% MeCN/H₂O to afford ethyl 2-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)acetate (28.2 mg, 23.43%) as a yellow solid. 1H NMR (400 MHz, DMSO-d6) δ 14.21-14.13 (m, 1H), 12.16 (s, 1H), 10.51 (s, 1H), 8.60 (d, J=5.7 Hz, 1H), 6.95 (d, J=7.8 Hz, 1H), 6.31 (s, 1H), 5.87 (d, J=2.1 Hz, 1H), 5.70 (d, J=2.2 Hz, 1H), 5.01-4.96 (m, 1H), 4.70 (s, 2H), 4.47 (d, J=2.8 Hz, 2H), 4.21-4.12 (m, 2H), 3.71 (s, 3H), 3.61-3.51 (m, 1H), 3.08-3.00 (m, 1H), 2.48-2.42 (m, 1H), 2.03-1.96 (m, 1H), 1.91-1.84 (m, 1H), 1.74-1.64 (m, 2H), 1.60-1.54 (m, 1H), 1.22 (t, J=7.1 Hz, 3H), 1.02 (d, J=6.5 Hz, 6H). MS (ESI) mass calcd. for C₂₆H₃₅N₅O₈, 545.25 m/z, found, 546.30 [M+H]⁺.

### Example 21.24: Preparation of Compound 64q: 2-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)ethane-1-sulfonic acid

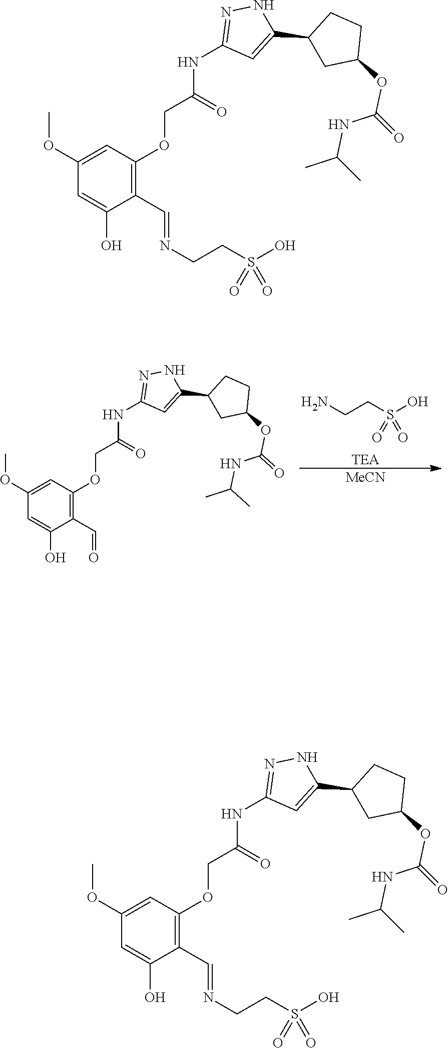

### Step 1: Synthesis of 2-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)ethane-1-sulfonic acid

(1R,3S)-3-{5-[2-(2-formyl-3-hydroxy-5-methoxyphenoxy)acetamido]-2H-pyrazol-3-yl}cyclopentyl N-isopropylcarbamate (100 mg, 0.217 mmol, 1 equiv.), 2-aminoethane-1-sulfonic acid (35.3 mg, 0.282 mmol, 1.3 equiv.), acetonitrile (5 mL) and a stir bar were added to a nitrogen-purged 8 mL vail and stirred until homogenous, then treated with NaOH (17.4 mg, 0.434 mmol, 2 equiv.). The resulting mixture was stirred overnight at 40° C. The reaction was checked by LCMS and showed complete consumption of starting material. The reaction mixture was purified by reverse phase preparatory HPLC, 0-25% ACN/H₂O to afford 2-(((E)-2-hydroxy-6-(2-((5-((1S,3R)-3-((isopropylcarbamoyl)oxy)cyclopentyl)-1H-pyrazol-3-yl)amino)-2-oxoethoxy)-4-methoxybenzylidene)amino)ethane-1-sulfonic acid (47.0 mg, 0.083 mmol, 36.7% yield). 1H NMR (400 MHz, DMSO-d6) δ 14.03-13.89 (m, 1H), 12.17 (s, 1H), 10.45 (s, 1H), 8.53 (d, J=8.4 Hz, 1H), 6.98 (d, J=7.8 Hz, 1H), 6.32 (s, 1H), 5.72 (d, J=2.1 Hz, 1H), 5.54 (d, J=2.2 Hz, 1H), 5.03-4.94 (m, 1H), 4.65 (s, 2H), 3.81-3.72 (m, 2H), 3.67 (s, 3H), 3.62-3.52 (m, 1H), 3.11-2.98 (m, 1H), 2.74 (t, J=7.1 Hz, 2H), 2.35-2.31 (m, 1H), 2.05-1.94 (m, 1H), 1.94-1.80 (m, 1H), 1.78-1.64 (m, 2H), 1.62-1.52 (m, 1H), 1.02 (d, J=6.5 Hz, 6H). MS (ESI) mass calcd. for C₂₄H₃₃N₅O₉S, 567.2 m/z, found [M+H]⁺ 568.20 m/z.

### Example 21.25: Preparation of Compound 235a: (1R,3S)-3-(3-(2-(3-hydroxy-2-((E)-(isopropylimino)methyl)-5-methoxyphenoxy)acetamido)-1H-pyrazol-5-yl)cyclopentyl 6-oxa-3-azabicyclo[3.1.1]heptane-3-carboxylate

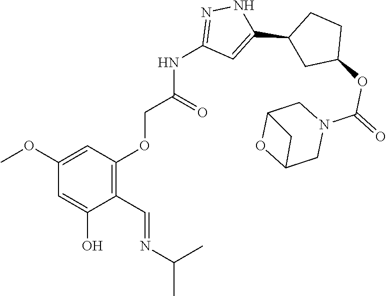 [SMILES: COc1cc(O)c(/C=N/C(C)C)c(OCC(=O)Nc2cc([C@H]3CC[C@@H](OC(=O)N4CC5CC(C4)O5)C3)[nH]n2)c1]

### Synthetic Route

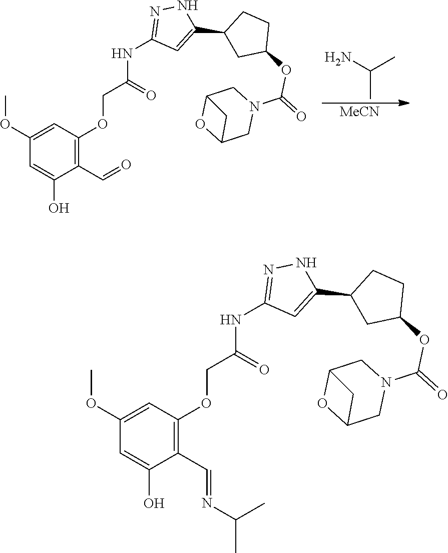

### Step 1: Synthesis of (1R,3S)-3-(3-(2-(3-hydroxy-2-((E)-(isopropylimino)methyl)-5-methoxyphenoxy)acetamido)-1H-pyrazol-5-yl)cyclopentyl 6-oxa-3-azabicyclo[3.1.1]heptane-3-carboxylate

To a solution of (1R,3S)-3-{5-[2-(2-formyl-3-hydroxy-5-methoxyphenoxy)acetamido]-2H-pyrazol-3-yl}cyclopentyl 6-oxa-3-azabicyclo[3.1.1]heptane-3-carboxylate (70 mg, 0.140 mmol, 1 equiv) in MeCN (2 mL) was added isopropylamine (24.8 mg, 0.420 mmol, 3 equiv). After stirring for 2 h at room temperature, the resulting mixture was concentrated under reduced pressure. The crude product was purified by reverse phase chromatography, 10 to 50% MeCN/H₂O to afford (1R,3S)-3-[5-(2-{3-hydroxy-2-[(1E)-(isopropylimino)methyl]-5-methoxyphenoxy}acetamido)-2H-pyrazol-3-yl]cyclopentyl 6-oxa-3-azabicyclo[3.1.1]heptane-3-carboxylate (28.6 mg, 37% yield) as a yellow solid. MS (ESI) calcd. for C₂₇H₃₅N₅O₇, 541.25 m/z, found [M+H]+ 542.20 m/z. 1H NMR (300 MHz, DMSO-d6) δ 14.47-14.43 (m, 1H), 12.17 (s, 1H), 10.49 (s, 1H), 8.59 (d, J=7.5 Hz, 1H), 6.36-6.34 (m, 1H), 5.78 (d, J=1.8 Hz, 1H), 5.61 (d, J=1.85 Hz, 1H), 5.12-5.09 (m, 1H), 4.67 (s, 2H), 4.55-4.50 (m, 1H), 4.48-4.43 (m, 1H), 3.77-3.71 (m, 1H), 3.68 (s, 3H), 3.51-3.40 (m, 4H), 3.16-3.10 (m, 1H), 3.05-2.94 (m, 1H), 2.45-2.32 (m, 1H), 2.08-1.72 (m, 6H), 1.28-1.25 (m, 6H).

### Example 21.26: Preparation of Compound 224a: (1R,3S)-3-[5-(2-{3-hydroxy-2-[(1E)-(isopropylimino)methyl]-5-methoxyphenoxy}acetamido)-2H-pyrazol-3-yl]cyclopentyl 1,2-oxazolidine-2-carboxylate

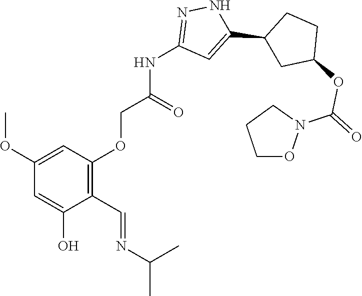 [SMILES: COc1cc(O)c(/C=N/C(C)C)c(OCC(=O)Nc2cc([C@H]3CC[C@@H](OC(=O)N4CCCO4)C3)[nH]n2)c1]

### Synthetic Route

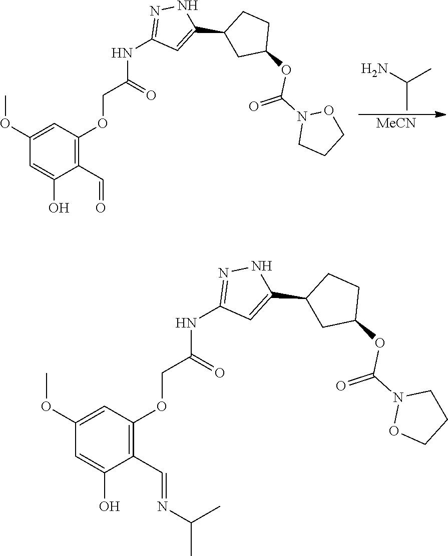

### Step 1: Synthesis of (1R,3S)-3-[5-(2-{3-hydroxy-2-[(1E)-(isopropylimino)methyl]-5-methoxyphenoxy}acetamido)-2H-pyrazol-3-yl]cyclopentyl 1,2-oxazolidine-2-carboxylate

To a stirred solution of (1R,3S)-3-{5-[2-(2-formyl-3-hydroxy-5-methoxyphenoxy)acetamido]-2H-pyrazol-3-yl}cyclopentyl 1,2-oxazolidine-2-carboxylate (60.0 mg, 0.126 mmol, 1 equiv) in acetonitrile (2 mL) was added isopropylamine (32 μL, 0.378 mmol, 3 equiv) at rt. The resulting mixture was stirred for 3 h. The resulting mixture was concentrated under reduced pressure. The residue was purified by reverse phase chromatography (10-46% ACN/H₂O). This resulted in (1R,3S)-3-[5-(2-{3-hydroxy-2-[(1E)-(isopropylimino)methyl]-5-methoxyphenoxy}acetamido)-2H-pyrazol-3-yl]cyclopentyl 1,2-oxazolidine-2-carboxylate (28.0 mg, 42.26% yield) as a light yellow solid. MS (ESI) calcd. for C₂₅H₃₃N₅O₇, 515.24 m/z, found [M+H]⁺ 516.35 m/z. 1H NMR (400 MHz, DMSO-d6) δ14.47-14.44 (m, 1H), 12.17 (s, 1H), 10.49 (s, 1H), 8.60-8.58 (m, 1H), 6.33 (s, 1H), 5.78-5.77 (m, 1H), 5.61-5.60 (m, 1H), 5.07-5.06 (m, 1H), 4.68 (s, 2H), 3.77-3.75 (m, 3H), 3.74 (s, 3H), 3.50-3.46 (m, 2H), 3.11-3.09 (m, 1H), 2.49-2.46 (m, 1H), 2.16-2.10 (m, 2H), 2.08-2.02 (m, 1H), 1.91-1.90 (m, 1H), 1.80-1.70 (m, 3H), 1.27-1.25 (m, 6H).

### Example 21.27: Preparation of Compound 180a: (1R,3S)-3-[5-(2-{3-hydroxy-2-[(1E)-(isopropylimino)methyl]-5-methoxyphenoxy}acetamido)-2H-pyrazol-3-yl]cyclopentyl N,N-diethylcarbamate

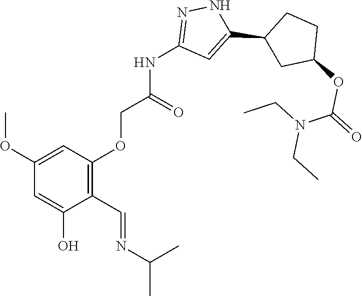 [SMILES: CCN(CC)C(=O)O[C@@H]1CC[C@H](c2cc(NC(=O)COc3cc(OC)cc(O)c3/C=N/C(C)C)n[nH]2)C1]

### Synthetic Route

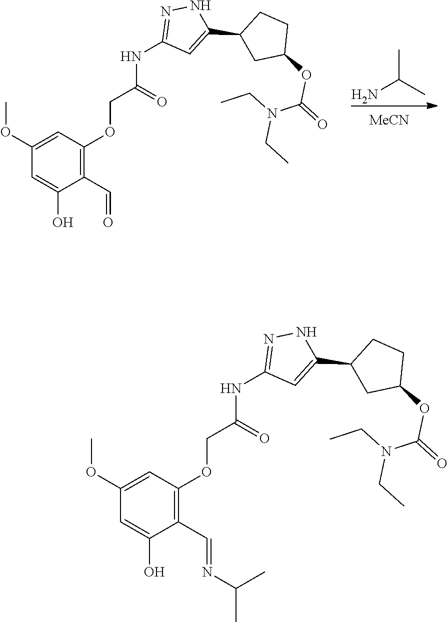

### Step 1: Synthesis of (1R,3S)-3-[5-(2-{3-hydroxy-2-[(1E)-(isopropylimino)methyl]-5-methoxyphenoxy}acetamido)-2H-pyrazol-3-yl]cyclopentyl N,N-diethylcarbamate

To a stirred solution of (1R,3S)-3-{5-[2-(2-formyl-3-hydroxy-5-methoxyphenoxy)acetamido]-2H-pyrazol-3-yl}cyclopentyl N,N-diethylcarbamate (80 mg, 0.169 mmol, 1 equiv.) in acetonitrile (5 mL) was added isopropylamine (29.90 mg, 0.507 mmol, 3 equiv.) at room temperature. The resulting mixture was stirred at room temperature for 3 h. The resulting mixture was concentrated under vacuum. The residue was purified by C18 reverse phase chromatography (10-60% MeCN/H₂O) to afford (1R,3S)-3-[5-(2-{3-hydroxy-2-[(1E)-(isopropylimino)methyl]-5-methoxyphenoxy}acetamido)-2H-pyrazol-3-yl]cyclopentyl N,N-diethylcarbamate (33.8 mg, 38.77% yield) as a light green solid. 1H NMR (400 MHz, DMSO-d6) δ (ppm): 14.46-14.43 (m, 1H), 12.16 (s, 1H), 10.48 (s, 1H), 8.59 (d, J=7.6 Hz, 1H), 6.33-6.31 (m, 1H), 5.78 (d, J=2.0 Hz, 1H), 5.61 (d, J=1.6 Hz, 1H), 5.04-5.02 (m, 1H), 4.67 (s, 2H), 3.75-3.72 (m, 1H), 3.68 (s, 3H), 3.33-3.08 (m, 5H), 2.44-2.40 (m, 1H), 2.03-2.01 (m, 1H), 1.92-1.88 (m, 1H), 1.78-1.67 (m, 3H), 1.26 (d, J=6.4 Hz, 6H), 1.03-0.99 (m, 6H). MS(ESI) calcd for C₂₆H₃₇N₅O₆ 515.27 m/z, found [M+H]+ 516.40 m/z.

### Example 21.28: Preparation of Compound 253a: (1R,3S)-3-(3-(2-(3-hydroxy-2-((E)-(isopropylimino)methyl)-5-methoxyphenoxy)acetamido)-1H-pyrazol-5-yl)cyclopentyl prop-2-yn-1-ylcarbamate

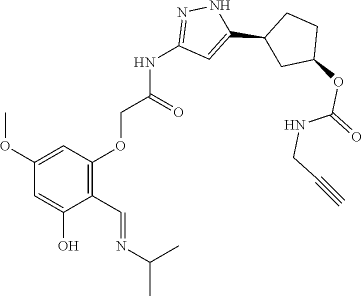 [SMILES: C#CCNC(=O)O[C@@H]1CC[C@H](c2cc(NC(=O)COc3cc(OC)cc(O)c3/C=N/C(C)C)n[nH]2)C1]

### Synthetic Route

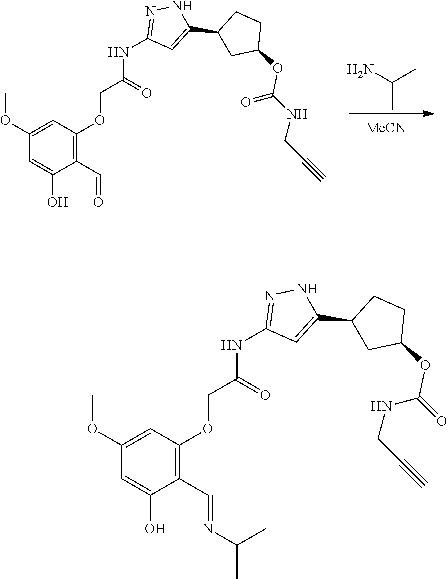

### Step 1: Synthesis of (1R,3S)-3-(3-(2-(3-hydroxy-2-((E)-(isopropylimino)methyl)-5-methoxyphenoxy)acetamido)-1H-pyrazol-5-yl)cyclopentyl prop-2-yn-1-ylcarbamate

To a solution of (1R,3S)-3-{5-[2-(2-formyl-3-hydroxy-5-methoxyphenoxy)acetamido]-2H-pyrazol-3-yl}cyclopentyl N-(prop-2-yn-1-yl)carbamate (50 mg, 0.110 mmol, 1 equiv) in MeCN (2 mL) was added isopropylamine (32.3 mg, 0.550 mmol, 5 equiv). After stirring for 2 h at room temperature, the resulting mixture was concentrated under reduced pressure. The crude product was purified by reverse flash chromatography (10-60% MeCN/H₂O) to afford (1R,3S)-3-[5-(2-{3-hydroxy-2-[(1E)-(isopropylimino)methyl]-5-methoxyphenoxy}acetamido)-2H-pyrazol-3-yl]cyclopentyl N-(prop-2-yn-1-yl)carbamate (32.8 mg, 58% yield) as a white solid. MS (ESI) calcd. for C₂₅H₃₁N₅O₆, 497.23 m/z, found [M+H]⁺ 498.15 m/z. 1H NMR (400 MHz, DMSO-d6) δ 14.47-14.44 (m, 1H), 12.17 (s, 1H), 10.49 (s, 1H), 8.60 (d, J=7.2 Hz, 1H), 7.55 (t, J=5.2 Hz, 1H), 6.31 (s, 1H), 5.78 (d, J=2.0 Hz, 1H), 5.61 (d, J=1.6 Hz, 1H), 5.03-5.00 (m, 1H), 4.68 (s, 2H), 3.79-3.71 (m, 3H), 3.69 (s, 3H), 3.08-3.04 (m, 2H), 2.48-2.45 (m, 1H), 2.04-1.98 (m, 1H), 197-1.88 (m, 1H), 1.74-1.66 (m, 2H), 1.62-1.55 (m, 1H), 1.27-1.26 (m, 6H).

### Example 21.29: Preparation of Compound 82a: (1R,3S)-3-(3-(2-(3-hydroxy-2-((E)-(isopropylimino)methyl)phenoxy)acetamido)-1H-pyrazol-5-yl)cyclopentyl (1-methylcyclopropyl)carbamate

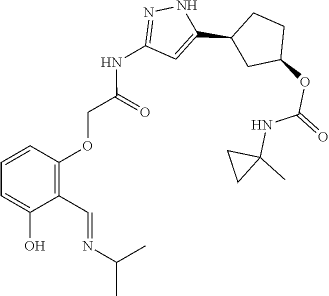 [SMILES: CC(C)/N=C/c1c(O)cccc1OCC(=O)Nc1cc([C@H]2CC[C@@H](OC(=O)NC3(C)CC3)C2)[nH]n1]

### Synthetic Route

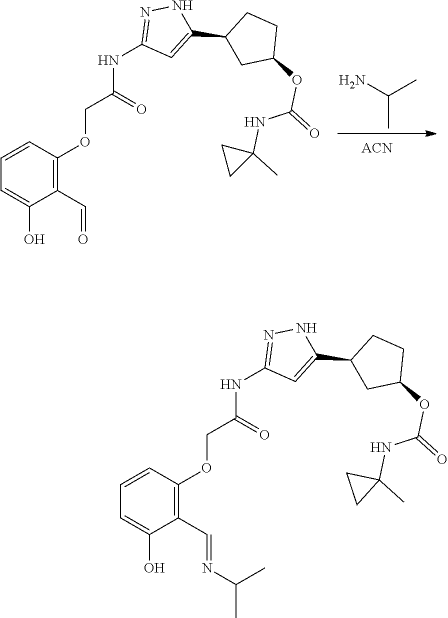

### Step 1: Synthesis of (1R,3S)-3-(3-(2-(3-hydroxy-2-((E)-(isopropylimino)methyl)phenoxy)acetamido)-1H-pyrazol-5-yl)cyclopentyl (1-methylcyclopropyl)carbamate

To a stirred suspension of (1R,3S)-3-(3-(2-(2-formyl-3-hydroxyphenoxy)acetamido)-1H-pyrazol-5-yl)cyclopentyl (1-methylcyclopropyl)carbamate (100.0 mg, 0.22 mmol, 1.0 equiv) in 3 mL ACN was added propan-2-amine (16.0 mg, 0.27 mmol, 1.2 equiv) at 25° C. The resulting mixture was stirred for additional 1 h, concentrated in vacuo and purified by Prep-HPLC, 24 to 46% ACN/H₂O to afford (1R,3S)-3-(3-(2-(3-hydroxy-2-((E)-(isopropylimino)methyl)phenoxy)acetamido)-1H-pyrazol-5-yl)cyclopentyl (1-methylcyclopropyl)carbamate (35.2 mg, 31.99% yield) as a yellow solid. 1H NMR (400 MHz, DMSO) δ 14.66 (s, 1H), 12.15 (s, 1H), 10.51 (s, 1H), 8.92 (s, 1H), 7.36 (s, 1H), 7.33-7.21 (m, 1H), 6.42 (d, J=8.4 Hz, 1H), 6.28 (d, J=8.7 Hz, 2H), 4.97 (s, 1H), 4.73 (s, 2H), 3.75-3.66 (m, 1H), 3.04 (d, J=8.8 Hz, 1H), 2.44-2.30 (m, 1H), 1.98 (d, J=9.4 Hz, 1H), 1.87 (s, 1H), 1.67 (s, 2H), 1.54 (s, 1H), 1.28-1.20 (m, 9H), 0.58 (s, 2H), 0.55-0.46 (m, 2H), MS (ESI) calcd. for C₂₅H₃₃N₅O₅, 483.20 m/z, found [M+H]⁺ 484.35 m/z.

### Example 21.30: Preparation of Compound 221a: (1R,3S)-3-(3-(2-(3-hydroxy-2-((E)-(isopropylimino)methyl)-5-methoxyphenoxy)acetamido)-1H-pyrazol-5-yl)cyclopentyl propylcarbamate

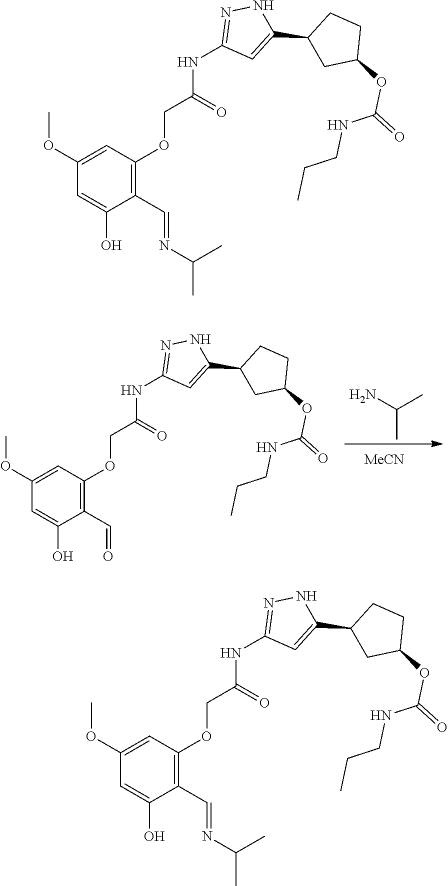

### Step 1: Synthesis of (1R,3S)-3-(3-(2-(3-hydroxy-2-((E)-(isopropylimino)methyl)-5-methoxyphenoxy)acetamido)-1H-pyrazol-5-yl)cyclopentyl propylcarbamate

To a stirred solution of (1R,3S)-3-{5-[2-(2-formyl-3-hydroxy-5-methoxyphenoxy)acetamido]-2H-pyrazol-3-yl}cyclopentyl N-propylcarbamate (50 mg, 0.109 mmol, 1 equiv) in acetonitrile (3 mL) was added isopropylamine (32.09 mg, 0.545 mmol, 5.0 equiv) in portions at room temperature. The resulting mixture was stirred for 2 h at room temperature. The resulting mixture was concentrated under reduced pressure. The crude product was purified by Prep-HPLC (5-60% MeCN/H₂O) to afford (1R,3S)-3-[5-(2-{3-hydroxy-2-[(1E)-(isopropylimino)methyl]-5-methoxyphenoxy}acetamido)-2H-pyrazol-3-yl]cyclopentyl N-propylcarbamate (32.5 mg, 58.57% yield) as a yellow solid. 1H NMR (300 MHz, DMSO-d6) δ 12.50-11.60 (m, 2H), 10.72 (s, 1H), 8.84-8.19 (m, 1H), 7.06-7.02 (m, 1H), 6.31-6.08 (m, 3H), 5.10-4.93 (m, 3H), 4.18-4.03 (m, 1H), 3.84 (s, 3H), 3.15-3.03 (m, 1H), 3.00-2.87 (m, 2H), 2.44-2.38 (m, 1H), 2.05-1.92 (m, 1H), 1.89-1.83 (m, 1H), 1.78-1.72 (m, 2H), 1.70-1.59 (m, 1H), 1.57-1.30 (m, 8H), 0.90-0.75 (m, 3H). MS (ESI) calcd. for C₂₅H₃₅N₅O₆ 501.26 m/z, found [M+H]⁺ 502.35 m/z.

### Example 21.31: Preparation of Compound 223a: (1s,3s)-3-(3-(2-(3-hydroxy-2-((E)-(isopropylimino)methyl)-5-methoxyphenoxy)acetamido)-1H-pyrazol-5-yl)cyclobutyl isopropylcarbamate

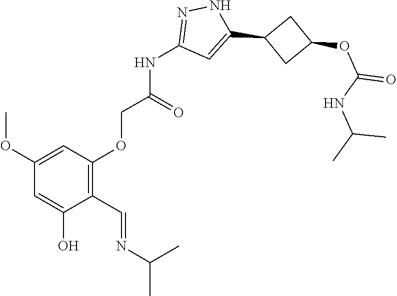 [SMILES: COc1cc(O)c(/C=N/C(C)C)c(OCC(=O)Nc2cc([C@H]3C[C@@H](OC(=O)NC(C)C)C3)[nH]n2)c1]

### Synthetic Route

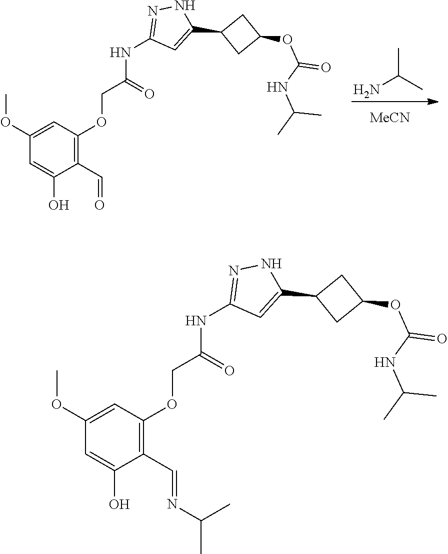

### Step 1: Synthesis of (1s,3s)-3-(3-(2-(3-hydroxy-2-((E)-(isopropylimino)methyl)-5-methoxyphenoxy)acetamido)-1H-pyrazol-5-yl)cyclobutyl isopropylcarbamate

(1s,3s)-3-{5-[2-(2-formyl-3-hydroxy-5-methoxyphenoxy)acetamido]-2H-pyrazol-3-yl}cyclobutyl N-isopropylcarbamate (200 mg, 0.448 mmol, 1 equiv.), isopropylamine (132.40 mg, 2.240 mmol, 5 equiv.), a stir bar and MeCN (5 mL) were added to a 20 mL vial and stirred until homogenous. The resulting mixture was stirred for 2 h at rt., then concentrated under vacuum. The residue was purified by reverse-phase chromatography (5-40% ACN/H₂O) to afford (1s,3s)-3-[5-(2-{3-hydroxy-2-[(1E)-(isopropylimino)methyl]-5-methoxyphenoxy}acetamido)-2H-pyrazol-3-yl]cyclobutyl N-isopropylcarbamate (68.4 mg, 30.41% yield) as a yellow solid. MS (ESI) calcd. for C₂₄H₃₃N₅O₆ 487.24 m/z, found [M+H]⁺ 488.15. 1H NMR (400 MHz, DMSO-d6) δ 14.26 (s, 1H), 12.25 (s, 1H), 10.52 (s, 1H), 8.61 (d, J=7.5 Hz, 1H), 7.07 (d, J=7.8 Hz, 1H), 6.35 (s, 1H), 5.81 (d, J=2.1 Hz, 1H), 5.65 (d, J=2.2 Hz, 1H), 4.86-4.76 (m, 1H), 4.70 (s, 2H), 3.85-3.74 (m, 1H), 3.70 (s, 3H), 3.63-3.47 (m, 1H), 3.14-2.98 (m, 1H), 2.75-2.57 (m, 2H), 2.14-1.95 (m, 2H), 1.28 (d, J=6.5 Hz, 6H), 1.04 (d, J=6.6 Hz, 6H).

### Example 21.32: Preparation of Compound 251a: (1R,3S)-3-(3-(2-(3-hydroxy-2-((E)-(isopropylimino)methyl)-5-methoxyphenoxy)acetamido)-1H-pyrazol-5-yl)cyclopentyl (1-methylcyclopropyl)carbamate

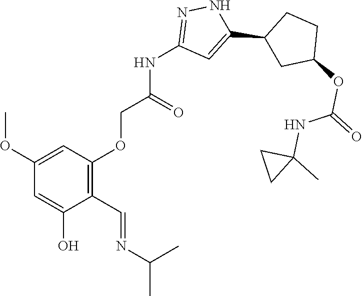 [SMILES: COc1cc(O)c(/C=N/C(C)C)c(OCC(=O)Nc2cc([C@H]3CC[C@@H](OC(=O)NC4(C)CC4)C3)[nH]n2)c1]

### Synthetic Route

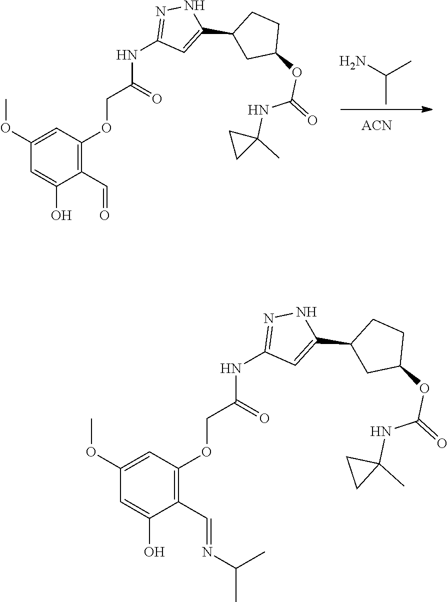

### Step 1: (1R,3S)-3-(3-(2-(3-hydroxy-2-((E)-(isopropylimino)methyl)-5-methoxyphenoxy)acetamido)-1H-pyrazol-5-yl)cyclopentyl (1-methylcyclopropyl)carbamate

A suspension of (1R,3S)-3-(3-(2-(2-formyl-3-hydroxy-5-methoxyphenoxy)acetamido)-1H-pyrazol-5-yl)cyclopentyl (1-methylcyclopropyl)carbamate (110 mg, 0.233 mmol, 1 equiv) and propan-2-amine (0.28 mL, 0.280 mmol, 1.2 equiv) in ACN (1 mL) was stirred for 1 h at room temperature. The solid was collected by filter and washed with ACN (3×1 mL). The resulting solid was lyophilized to afford (1R,3S)-3-[5-(2-{3-hydroxy-2-[(1E)-(isopropylimino) methyl]-5-methoxyphenoxy}acetamido)-2H-pyrazol-3-yl]cyclopentyl N-(1-methylcyclopropyl) carbamate (32.9 mg, 25.7% yield) as a green solid. 1H NMR (300 MHz, DMSO) δ 12.24 (s, 1H), 10.43 (s, 1H), 10.30 (s, 1H), 7.57 (t, J=8.5 Hz, 1H), 7.15 (d, J=5.9 Hz, 1H), 6.81 (d, J=8.5 Hz, 1H), 6.70 (d, J=8.5 Hz, 1H), 6.36 (s, 1H), 4.80 (d, J=7.5 Hz, 1H), 4.75 (s, 2H), 3.86 (s, 3H), 3.05 (q, J=9.0 Hz, 1H), 2.90 (q, J=6.6 Hz, 2H), 2.65 (d, J=8.4 Hz, 2H), 2.05 (q, J=9.9 Hz, 2H), 1.36 (p, J=7.3 Hz, 2H), 0.81 (t, J=7.4 Hz, 3H), MS (ESI) calcd. for C₂₆H₃₅N₅O₆, 513.26 m/z, found [M+H]⁺ 514.25 m/z.

### Example 21.33: Preparation of Compound 64a: (1R,3S)-3-(3-(2-(3-hydroxy-2-((E)-(isopropylimino)methyl)-5-methoxyphenoxy)acetamido)-1H-pyrazol-5-yl)cyclopentyl isopropylcarbamate

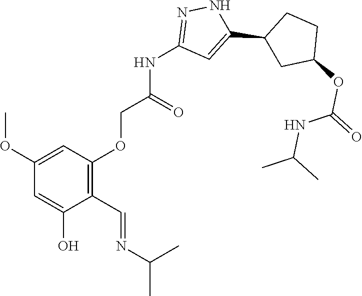 [SMILES: COc1cc(O)c(/C=N/C(C)C)c(OCC(=O)Nc2cc([C@H]3CC[C@@H](OC(=O)NC(C)C)C3)[nH]n2)c1]

### Synthetic Route

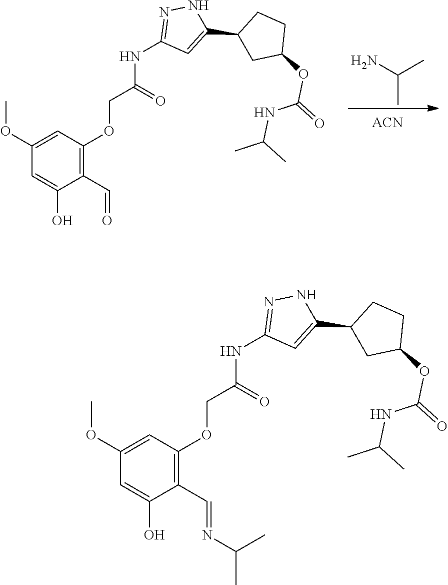

### Step 1: (1R,3S)-3-(3-(2-(3-hydroxy-2-((E)-(isopropylimino)methyl)-5-methoxyphenoxy)acetamido)-1H-pyrazol-5-yl)cyclopentyl isopropylcarbamate

A suspension of (1R,3S)-3-{5-[2-(2-formyl-3-hydroxy-5-methoxyphenoxy)acetamido]-2H-pyrazol-3-yl}cyclopentyl N-isopropylcarbamate (6.5 g, 14.115 mmol, 1.0 equiv) and propan-2-amine (2.9 g, 49.403 mmol, 3.5 equiv) in ACN (50 mL) was stirred for 4 h at 25° C. The solid was filtered and washed with ACN (3×5 mL) and lyophilized to afford (1R,3S)-3-(3-(2-(3-hydroxy-2-((E)-(isopropylimino)methyl)-5-methoxyphenoxy)acetamido)-1H-pyrazol-5-yl)cyclopentyl isopropylcarbamate (6.9 g, 95.6% yield) as a yellow solid. 1H NMR (300 MHz, DMSO) δ 14.46 (dd, J=7.4, 4.7 Hz, 1H), 12.16 (s, 1H), 10.49 (s, 1H), 8.60 (d, J=7.3 Hz, 1H), 6.95 (d, J=7.8 Hz, 1H), 6.32 (s, 1H), 5.79 (d, J=2.1 Hz, 1H), 5.62 (d, J=2.2 Hz, 1H), 4.99 (s, 1H), 4.68 (s, 2H), 3.81-3.64 (m, 4H), 3.63-3.49 (m, 1H), 3.17-2.96 (m, 1H), 2.51-2.42 (m, 1H), 2.09-1.91 (m, 1H), 1.93-1.80 (m, 1H), 1.79-1.47 (m, 3H), 1.27 (d, J=6.4 Hz, 6H), 1.03 (d, J=6.6 Hz, 6H), MS (ESI) calcd for C₂₅H₃₅N₅O₆, 501.26 m/z, found [M+H]⁺ 502.25 m/z.

### Example 21.34: Preparation of Compound 231a: (1R,3S)-3-(3-(2-(3-hydroxy-2-((E)-(isopropylimino)methyl)-5-methoxyphenoxy)acetamido)-1H-pyrazol-5-yl)cyclopentyl 3-oxa-6-azabicyclo[3.1.1]heptane-6-carboxylate

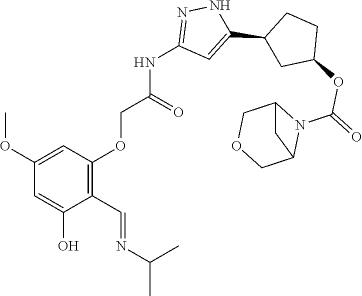 [SMILES: COc1cc(O)c(/C=N/C(C)C)c(OCC(=O)Nc2cc([C@H]3CC[C@@H](OC(=O)N4C5COCC4C5)C3)[nH]n2)c1]

### Synthetic Route

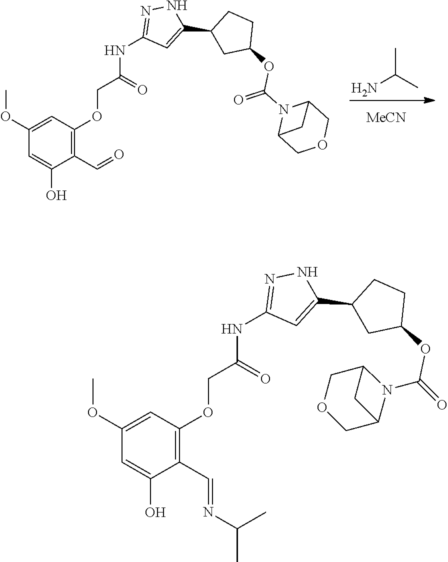

### Step 1: Synthesis of Compound 231a (1R,3S)-3-(3-(2-(3-hydroxy-2-((E)-(isopropylimino)methyl)-5-methoxyphenoxy)acetamido)-1H-pyrazol-5-yl)cyclopentyl 3-oxa-6-azabicyclo[3.1.1]heptane-6-carboxylate

To a stirred solution of (1R,3S)-3-{5-[2-(2-formyl-3-hydroxy-5-methoxyphenoxy)acetamido]-2H-pyrazol-3-yl}cyclopentyl 3-oxa-6-azabicyclo[3.1.1]heptane-6-carboxylate (200 mg, 0.4 mmol, 1 equiv.) in MeCN (5 mL) was added isopropylamine (0.17 mL, 2.0 mmol, 5 equiv.) at room temperature. The resulting mixture was stirred for 4 h at room temperature. The resulting mixture was concentrated under reduced pressure. The residue was purified by C18 reverse phase chromatography (5-60% MeCN/H₂O) to afford (1R,3S)-3-(3-(2-(3-hydroxy-2-((E)-(isopropylimino)methyl)-5-methoxyphenoxy)acetamido)-1H-pyrazol-5-yl)cyclopentyl 3-oxa-6-azabicyclo[3.1.1]heptane-6-carboxylate (115.2 mg, 53.04% yield) as a light green solid. MS (ESI) calcd. for C₂₇H₃₅N₅O₇, 541.25 m/z, found [M+H]⁺ 542.35 m/z. 1H NMR (300 MHz, DMSO-d6) δ 14.47-14.43 (m, 1H), 12.16 (s, 1H), 10.48 (s, 1H), 8.59 (d, J=7.2 Hz, 1H), 6.32 (s, 1H), 5.78-5.77 (m, 1H), 5.61-5.60 (m, 1H), 5.10-5.08 (m, 1H), 4.67 (s, 2H), 4.04-4.02 (m, 3H), 3.92-3.79 (m, 1H), 3.77-3.71 (m, 1H), 3.68 (s, 3H), 3.65-3.62 (m, 2H), 3.14-3.09 (m, 1H), 2.45-2.42 (m, 2H), 2.07-1.86 (m, 2H), 1.65-1.54 (m, 3H), 1.58-1.56 (m, 1H), 1.28 (s, 3H), 1.26 (s, 3H).

### Example 21.35: Preparation of Compound 283a: (1R,3S)-3-(3-((*)-5-hydroxy-4-((E)-(isopropylimino)methyl)-7-methoxy-2,3-dihydro-1H-indene-2-carboxamido)-1H-pyrazol-5-yl)cyclopentyl tert-butylcarbamate

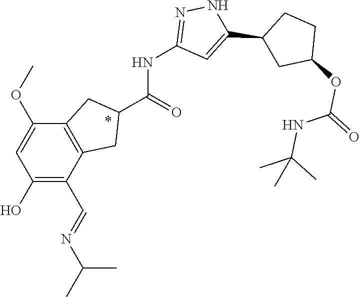 [SMILES: COc1cc(O)c(/C=N/C(C)C)c2c1CC(C(=O)Nc1cc([C@H]3CC[C@@H](OC(=O)NC(C)(C)C)C3)[nH]n1)C2]

### Synthetic Route

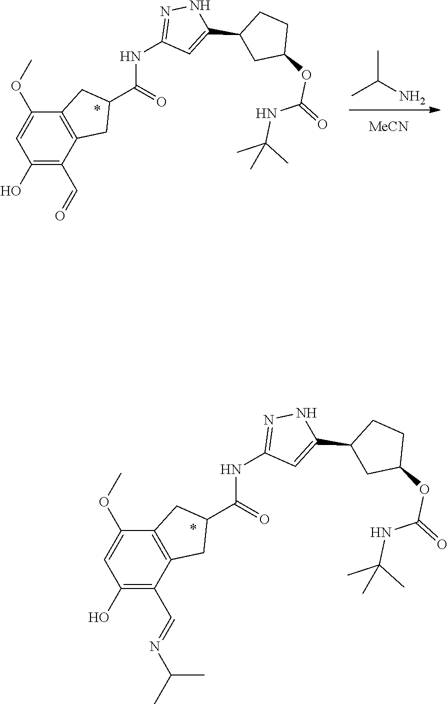

### Step 1: (1R,3S)-3-(3-((S)-5-hydroxy-4-((E)-(isopropylimino)methyl)-7-methoxy-2,3-dihydro-1H-indene-2-carboxamido)-1H-pyrazol-5-yl)cyclopentyl tert-butylcarbamate

(1R,3S)-3-(3-((S)-4-formyl-5-hydroxy-7-methoxy-2,3-dihydro-1H-indene-2-carboxamido)-1H-pyrazol-5-yl)cyclopentyl tert-butylcarbamate (48 mg, 0.099 mmol), and isopropylamine (17.6 mg, 0.297 mmol) in acetonitrile (2 mL) were stirred overnight in a nitrogen-purged 8 mL reaction vial. The next day the reaction mixture was purified by reverse-phase chromatography, 5 to 50% MeCN/H₂O to afford (1R,3S)-3-(3-((*)-5-hydroxy-4-((E)-(isopropylimino)methyl)-7-methoxy-2,3-dihydro-1H-indene-2-carboxamido)-1H-pyrazol-5-yl)cyclopentyl tert-butylcarbamate (Compound 283a, 32.9 mg, 98.9% purity) as a yellow green solid. MS (ESI) mass calcd. for C₂₈H₃₉N₅O₅, 525.30 m/z, found 526.45 [M+H]⁺. 1H NMR (400 MHz, DMSO-d6) δ14.20 (s, 1H), 12.05 (s, 1H), 10.40 (s, 1H), 8.40 (d, J=3.2 Hz, 1H), 6.77 (s, 1H), 6.33 (s, 1H), 6.14 (s, 1H), 5.02-4.92 (m, 1H), 3.75 (s, 3H), 3.70-3.59 (m, 1H), 3.46-3.37 (m, 1H), 3.28-3.18 (m, 1H), 3.18-3.08 (m, 1H), 3.08-2.92 (m, 2H), 2.85-2.76 (m, 1H), 2.48-2.40 (m, 1H), 2.12-1.95 (m, 1H), 1.95-1.80 (m, 1H), 1.77-1.62 (m, 2H), 1.62-1.49 (m, 1H), 1.23 (d, J=6.3 Hz, 6H), 1.19 (s, 9H). * Denotes a stereocenter with an undetermined absolute stereochemistry of a single isomer.

### Example 21.36: Preparation of Compound 284a: (1R,3S)-3-(5-((*)-5-hydroxy-4-((E)-(isopropylimino)methyl)-7-methoxy-2,3-dihydro-1H-indene-2-carboxamido)-1H-pyrazol-3-yl)cyclopentyl 1,2,2-trimethylhydrazine-1-carboxylate

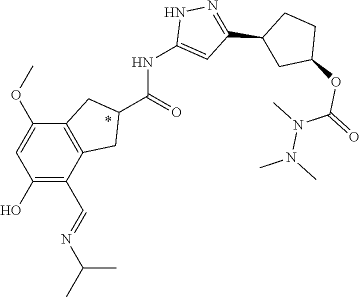 [SMILES: COc1cc(O)c(/C=N/C(C)C)c2c1CC(C(=O)Nc1cc([C@H]3CC[C@@H](OC(=O)N(C)N(C)C)C3)n[nH]1)C2]

### Synthetic Route

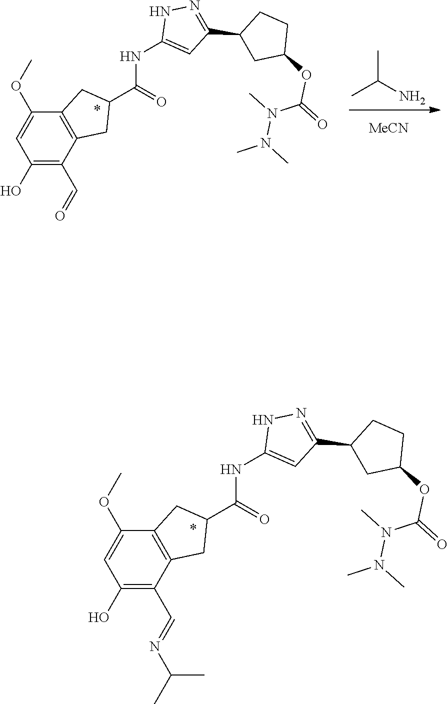

### Step 1: (1R,3S)-3-(5-((S)-5-hydroxy-4-((E)-(isopropylimino)methyl)-7-methoxy-2,3-dihydro-1H-indene-2-carboxamido)-1H-pyrazol-3-yl)cyclopentyl 1,2,2-trimethylhydrazine-1-carboxylate

(1R,3S)-3-(5-((S)-4-formyl-5-hydroxy-7-methoxy-2,3-dihydro-1H-indene-2-carboxamido)-1H-pyrazol-3-yl)cyclopentyl 1,2,2-trimethylhydrazine-1-carboxylate (11 g, 22.655 mmol, 1 equiv.), a stir bar and MeCN (200 mL) were added to a 500 mL flask, then treated with isopropylamine (6.70 g, 113.275 mmol, 5 equiv.). The resulting mixture was stirred at room temperature for 2 h and concentrated under reduced pressure. The residue was diluted with H₂O and extracted with DCM. The organic layers were washed with H₂O, dried over sodium sulphate, filtered and concentrated to dryness in vacuo to afford (1R,3S)-3-(5-((*)-5-hydroxy-4-((E)-(isopropylimino)methyl)-7-methoxy-2,3-dihydro-1H-indene-2-carboxamido)-1H-pyrazol-3-yl)cyclopentyl 1,2,2-trimethylhydrazine-1-carboxylate (Compound 284a, 9.6718 g, 81.06% yield). 1H NMR (400 MHz, DMSO-d6) δ 14.20 (s, 1H), 12.08 (s, 1H), 10.40 (s, 1H), 8.40 (s, 1H), 6.36 (s, 1H), 6.15 (s, 1H), 5.06 (s, 1H), 3.75 (s, 3H), 3.69-3.59 (m, 1H), 3.46-3.38 (m, 1H), 3.29-3.10 (m, 3H), 2.98 (dd, J=15.4, 9.0 Hz, 1H), 2.88-2.76 (m, 4H), 2.56-2.50 (m, 6H), 2.43-2.32 (m, 1H), 2.13-1.96 (m, 1H), 1.93-1.77 (m, 2H), 1.77-1.65 (m, 2H), 1.23 (d, J=6.4 Hz, 6H), MS (ESI) calcd. for C27H₃₈N₆O₅, 526.29 m/z, found: 527.35 [M+H]+. * Denotes a stereocenter with an undetermined absolute stereochemistry of a single isomer.

### Example 21.37: Preparation of Compound 284c: (1R,3S)-3-(5-((*)-4-((E)-(ethylimino)methyl)-5-hydroxy-7-methoxy-2,3-dihydro-1H-indene-2-carboxamido)-1H-pyrazol-3-yl)cyclopentyl 1,2,2-trimethylhydrazine-1-carboxylate

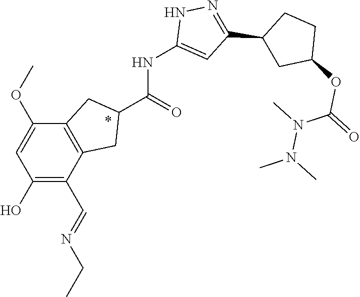 [SMILES: CC/N=C/c1c(O)cc(OC)c2c1CC(C(=O)Nc1cc([C@H]3CC[C@@H](OC(=O)N(C)N(C)C)C3)n[nH]1)C2]

### Synthetic Route

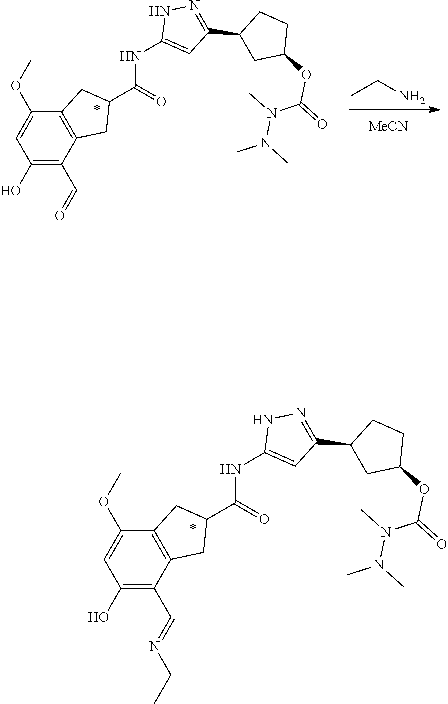

### Step 1: (1R,3S)-3-(5-((S)-4-((E)-(ethylimino)methyl)-5-hydroxy-7-methoxy-2,3-dihydro-1H-indene-2-carboxamido)-1H-pyrazol-3-yl)cyclopentyl 1,2,2-trimethylhydrazine-1-carboxylate

(1R,3S)-3-(5-((S)-4-formyl-5-hydroxy-7-methoxy-2,3-dihydro-1H-indene-2-carboxamido)-1H-pyrazol-3-yl)cyclopentyl 1,2,2-trimethylhydrazine-1-carboxylate (50 mg, 0.103 mmol, 1 equiv.), a stir bar and MeCN (2 mL) were added to a 50 mL flask, then treated with ethylamine (33 mg, 0.512 mmol, 4.98 equiv., 70%). The resulting mixture was stirred at r.t for 1 h, then concentrated. The residue was purified by reverse phase chromatography, 5 to 35% MeCN/H₂O to afford ((1R,3S)-3-(5-((*)-4-((E)-(ethylimino)methyl)-5-hydroxy-7-methoxy-2,3-dihydro-1H-indene-2-carboxamido)-1H-pyrazol-3-yl)cyclopentyl 1,2,2-trimethylhydrazine-1-carboxylate (Compound 284c, 27.4 mg, 49.52% yield). 1H NMR (400 MHz, DMSO-d6) δ 14.10 (s, 1H), 12.09 (s, 1H), 10.40 (s, 1H), 8.37 (d, J=3.1 Hz, 1H), 6.35 (s, 1H), 6.13 (s, 1H), 5.11-5.01 (m, 1H), 3.75 (s, 3H), 3.56 (q, J=7.2 Hz, 2H), 3.45-3.37 (m, 1H), 3.26-3.08 (m, 3H), 3.03-2.92 (m, 1H), 2.87-2.75 (m, 4H), 2.55-2.51 (m, 6H), 2.44-2.34 (m, 1H), 2.13-1.99 (m, 1H), 1.94-1.78 (m, 2H), 1.77-1.65 (m, 2H), 1.23 (t, J=7.2 Hz, 3H), MS (ESI) calcd. for C₂₆H₃₆N₆O₅, 512.28 m/z, found: 513.25 [M+H]+. * Denotes a stereocenter with an undetermined absolute stereochemistry of a single isomer.

### Example 21.38: Preparation of Compound 284b

### Synthesis of (1R,3S)-3-(5-((*)-5-hydroxy-7-methoxy-4-((Z)-(propylimino)methyl)-2,3-dihydro-1H-indene-2-carboxamido)-1H-pyrazol-3-yl)cyclopentyl 1,2,2-trimethylhydrazine-1-carboxylate

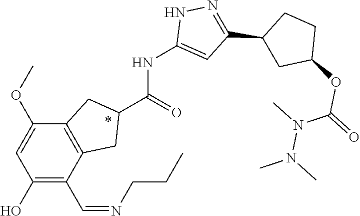 [SMILES: CCC/N=C\c1c(O)cc(OC)c2c1CC(C(=O)Nc1cc([C@H]3CC[C@@H](OC(=O)N(C)N(C)C)C3)n[nH]1)C2]

### Synthetic Route

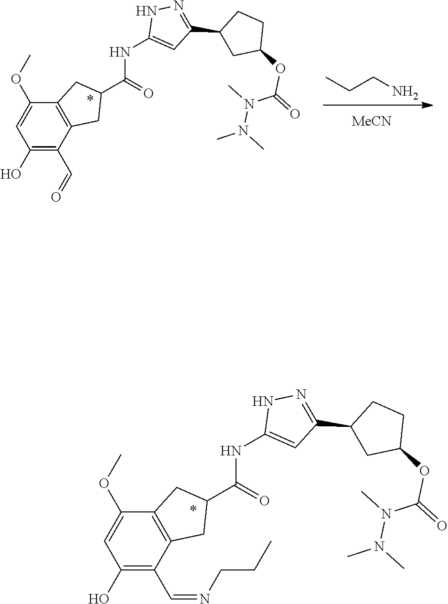

### Step 1: (1R,3S)-3-(5-((S)-5-hydroxy-7-methoxy-4-((Z)-(propylimino)methyl)-2,3-dihydro-1H-indene-2-carboxamido)-1H-pyrazol-3-yl)cyclopentyl 1,2,2-trimethylhydrazine-1-carboxylate

(1R,3S)-3-(5-((S)-4-formyl-5-hydroxy-7-methoxy-2,3-dihydro-1H-indene-2-carboxamido)-1H-pyrazol-3-yl)cyclopentyl 1,2,2-trimethylhydrazine-1-carboxylate (50 mg, 0.103 mmol, 1 equiv.), a stir bar and MeCN (2 mL) were added to a 50 mL flask, then treated with n-propylamine (30 mg, 0.508 mmol, 4.93 equiv.). The resulting mixture was stirred at r.t for 1 h, then concentrated. The residue was diluted with H₂O and extracted with DCM. The organic layers were washed with H₂O, dried over sodium sulphate, filtered and concentrated to dryness in vacuo to afford (1R,3S)-3-(5-((*)-5-hydroxy-7-methoxy-4-((Z)-(propylimino)methyl)-2,3-dihydro-1H-indene-2-carboxamido)-1H-pyrazol-3-yl)cyclopentyl 1,2,2-trimethylhydrazine-1-carboxylate (Compound 284b, 44.7 mg, 82.42% yield). 1H NMR (400 MHz, DMSO-d6) δ 14.17 (s, 1H), 12.09 (s, 1H), 10.40 (s, 1H), 8.37 (d, J=3.2 Hz, 1H), 6.35 (s, 1H), 6.13 (s, 1H), 5.09-5.00 (m, 1H), 3.75 (s, 3H), 3.50 (t, J=6.8 Hz, 2H), 3.44-3.38 (m, 1H), 3.27-3.07 (m, 3H), 3.03-2.91 (m, 1H), 2.88-2.73 (m, 4H), 2.55-2.51 (m, 6H), 2.44-2.35 (m, 1H), 2.13-2.01 (m, 1H), 1.91-1.79 (m, 2H), 1.75-1.67 (m, 2H), 1.66-1.59 (m, 2H), 0.92 (t, J=7.4 Hz, 3H), MS (ESI) calcd. for C₂₇H₃₈N₆O₅, 526.29 m/z, found: 527.35 [M+H]⁺. * Denotes a stereocenter with an undetermined absolute stereochemistry of a single isomer.

### Example 22: Preparation of Compounds 262 and 263

### Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2-methylpyrazolidine-1-carboxylate (Compound 262) and (1R,3S)-3-(3-((*)-4-formyl-5-hydroxy-7-methoxy-2,3-dihydro-H-indene-2-carboxamido)-1H-pyrazol-5-yl)cyclopentyl 2-methylpyrazolidine-1-carboxylate (Compound 263)

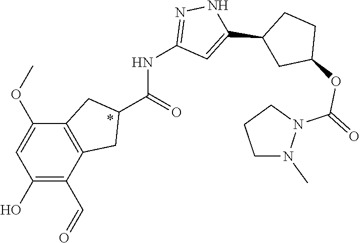 [SMILES: COc1cc(O)c(C=O)c2c1CC(C(=O)Nc1cc([C@H]3CC[C@@H](OC(=O)N4CCCN4C)C3)[nH]n1)C2]

### Synthetic Route

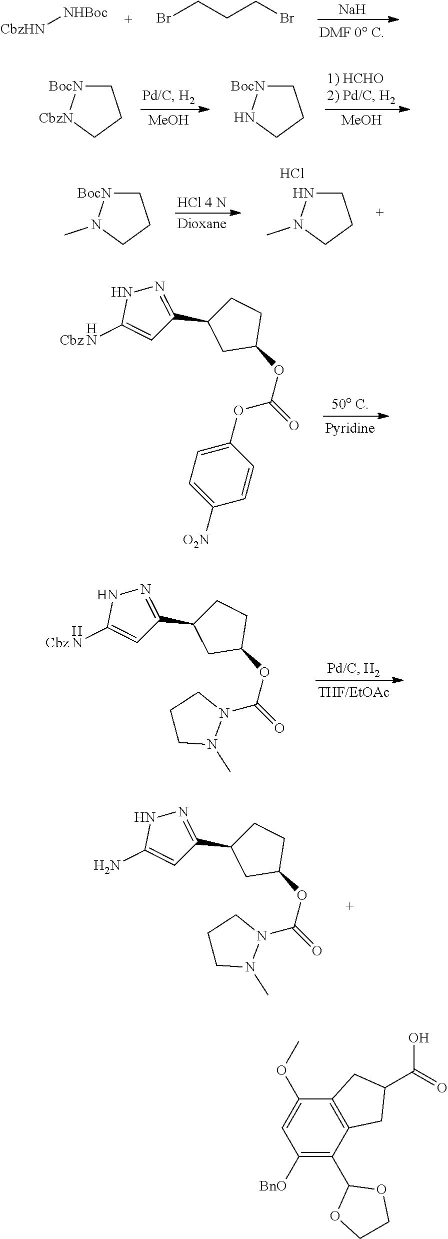

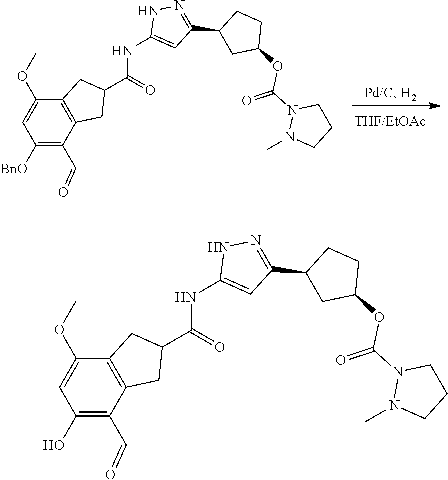 [SMILES: COc1cc(OCc2ccccc2)c(C=O)c2c1CC(C(=O)Nc1cc([C@H]3CC[C@@H](OC(=O)N4CCCN4C)C3)n[nH]1)C2 ; COc1cc(O)c(C=O)c2c1CC(C(=O)Nc1cc([C@H]3CC[C@@H](OC(=O)N4CCCN4C)C3)n[nH]1)C2]

### Step 1: Synthesis of 1-benzyl 2-(tert-butyl) pyrazolidine-1,2-dicarboxylate

To a stirred solution of N′-(tert-butoxycarbonyl)benzyloxycarbohydrazide (20 g, 75.104 mmol, 1 equiv) in DMF (20 mL) was added NaH (6.01 g, 150.208 mmol, 2 equiv, 60% in oil) at 0° C. The mixture was stirred for 30 min. 1,3-dibromopropane (15.16 g, 75.104 mmol, 1 equiv) was added and the mixture was allowed to warm to room temperature and stirred for 2 h. The reaction mixture was quenched with water and extracted with DCM (3×100 mL). The combined organic layers were washed with brine (3×100 mL), dried over anhydrous Na₂SO₄, filtered and concentrated under reduced pressure. The resulting residue was purified by silica gel column chromatography, 0 to 20% EtOAc/PE to provide 1-benzyl 2-(tert-butyl) pyrazolidine-1,2-dicarboxylate (20 g, 86% yield) as a light yellow oil.

### Step 2: Synthesis of tert-butyl pyrazolidine-1-carboxylate

To a solution of 1-benzyl 2-tert-butyl pyrazolidine-1,2-dicarboxylate (20 g, 65.282 mmol, 1 equiv) in methanol (20 mL) was added 10% Pd/C (20 g). The mixture was stirred at room temperature under hydrogen atmosphere for 1 h. The reaction mixture was filtered through a Celite pad and concentrated under reduced pressure to afford tert-butyl pyrazolidine-1-carboxylate (10 g, 89% yield). The crude material was used without any further purification.

### Step 3: Synthesis of tert-butyl 2-methylpyrazolidine-1-carboxylate

To a solution of tert-butyl pyrazolidine-1-carboxylate (10 g, 58.063 mmol, 1 equiv) in methanol (10 mL) was added HCHO 40% solution in H₂O (10.5 mL, 139.351 mmol, 2.4 equiv) and let stir for 1 h at room temperature. To the solution was added 10% Pd/C (13.29 g). The mixture was stirred for 2 h under hydrogen atmosphere. The reaction mixture was filtered through a Celite pad and concentrated under reduced pressure to afford tert-butyl 2-methylpyrazolidine-1-carboxylate (6.2 g, 89% yield) as a crude. The crude material was used without any further purification.

### Step 4: Synthesis of 1-methylpyrazolidine

Tert-butyl 2-methylpyrazolidine-1-carboxylate (6 g, 32.214 mmol, 1 equiv) was stirred at room temperature for 1 h in 4.0 M HCl in 1,4-dioxane (60 mL). The resulting mixture was concentrated under reduced pressure to afford 1-methylpyrazolidine hydrochloride (6 g) as off white solid. The crude material was used without any further purification.

### Step 5: Synthesis of (1R,3S)-3-(3-(((benzyloxy)carbonyl)amino)-1H-pyrazol-5-yl)cyclopentyl 2-methylpyrazolidine-1-carboxylate

To a solution of (1R,3S)-3-(5-{[(benzyloxy)carbonyl]amino}-2H-pyrazol-3-yl)cyclopentyl 4-nitrophenyl carbonate (6 g, 12.863 mmol, 1.00 equiv) in pyridine (60 mL) was added 1-methylpyrazolidine hydrochloride (1.58 g, 12.863 mmol, 1 equiv) and stirred for 1 h at 50° C. The resulting mixture was diluted with water and extracted with EtOAc (3×100 mL). The combined organic layers were washed with brine (3×100 mL), dried over anhydrous Na₂SO₄, filtered, and concentrated under reduced pressure. The mixture was purified by reverse phase column chromatography, 10 to 40% MeCN/H₂O to afford (1R,3S)-3-(5-{[(benzyloxy)carbonyl]amino}-2H-pyrazol-3-yl)cyclopentyl 2-methylpyrazolidine-1-carboxylate (1.1 g, 20.68% yield) as a yellow oil.

### Step 6: Synthesis of (1R,3S)-3-(3-amino-1H-pyrazol-5-yl)cyclopentyl 2-methylpyrazolidine-1-carboxylate

To a solution of (1R,3S)-3-(5-{[(benzyloxy)carbonyl]amino}-2H-pyrazol-3-yl)cyclopentyl 2-methylpyrazolidine-1-carboxylate (1 g, 2.419 mmol, 1 equiv) in ethyl acetate (5 mL) and THF (5 mL) was added 10% Pd/C (1.29 g). The mixture was stirred for 2 h under hydrogen atmosphere. The reaction mixture was filtered through a Celite pad and concentrated under reduced pressure to afford (1R,3S)-3-(5-amino-2H-pyrazol-3-yl)cyclopentyl 2-methylpyrazolidine-1-carboxylate (350 mg, 52% yield) as a white solid. The crude material was used without any further purification.

### Step 7: Synthesis of (1R,3S)-3-(3-(5-(benzyloxy)-4-formyl-7-methoxy-2,3-dihydro-1H-indene-2-carboxamido)-1H-pyrazol-5-yl)cyclopentyl 2-methylpyrazolidine-1-carboxylate

To a solution of (1R,3S)-3-(5-amino-2H-pyrazol-3-yl)cyclopentyl 2-methylpyrazolidine-1-carboxylate (320 mg, 1.146 mmol, 1 equiv) in pyridine (5 mL) was added 5-(benzyloxy)-4-(1,3-dioxolan-2-yl)-7-methoxy-2,3-dihydro-1H-indene-2-carboxylic acid (424 mg, 1.146 mmol, 1 equiv), EDCI (266 mg, 1.719 mmol, 1.5 equiv) at room temperature and stirred for 2 h. The resulting mixture was diluted with water and extracted with EtOAc (3×30 mL). The combined organic layers were washed with brine (3×30 mL), dried over anhydrous Na₂SO₄, filtered, and concentrated under reduced pressure. The mixture was purified by reverse phase column chromatography, 10 to 60% MeCN/H₂O to afford (1R,3S)-3-{5-[5-(benzyloxy)-4-formyl-7-methoxy-2,3-dihydro-1H-indene-2-amido]-2H-pyrazol-3-yl}cyclopentyl 2-methylpyrazolidine-1-carboxylate (300 mg, 44% yield) as a yellow oil.

### Step 8: Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2-methylpyrazolidine-1-carboxylate and (1R,3S)-3-(3-((*)-4-formyl-5-hydroxy-7-methoxy-2,3-dihydro-1H-indene-2-carboxamido)-1H-pyrazol-5-yl)cyclopentyl 2-methylpyrazolidine-1-carboxylate

To a solution of (1R,3S)-3-{5-[5-(benzyloxy)-4-formyl-7-methoxy-2,3-dihydro-1H-indene-2-amido]-2H-pyrazol-3-yl}cyclopentyl 2-methylpyrazolidine-1-carboxylate (290 mg, 0.493 mmol, 1 equiv) in EtOAc (3 mL) and THF (3 mL) was added 10% Pd/C (262 mg). The mixture was stirred for 1 h under hydrogen atmosphere. The reaction mixture was filtered through a Celite pad and concentrated under reduced pressure, The resulting crude material was purified by reverse phase column chromatography, 10 to 50% MeCN/H₂O with 0.05% NH₄HCO₃ modifier and chiral Prep-HPLC, Mobile Phase A: MTBE (0.5% 2M NH₃-MeOH), Mobile Phase B: MEOH:DCM=1:1 to afford (1R,3S)-3-(3-((*)-4-formyl-5-hydroxy-7-methoxy-2,3-dihydro-1H-indene-2-carboxamido)-1H-pyrazol-5-yl)cyclopentyl 2-methylpyrazolidine-1-carboxylate (Compound 262, 15.8 mg, 6.4% yield) as a white solid. 1H NMR (400 MHz, DMSO-d6) δ 12.08 (s, 1H), 11.24 (s, 1H), 10.43 (s, 1H), 10.07 (s, 1H), 6.39-6.34 (m, 2H), 5.05-5.03 (m, 1H), 3.84 (s, 3H), 3.49-3.38 (m, 5H), 3.17-3.11 (m, 1H), 2.99-2.92 (m, 1H), 2.88-2.80 (m, 3H), 2.42-2.35 (m, 4H), 2.08-2.01 (m, 3H), 1.88-1.65 (m, 4H). MS (ESI) calcd. for C₂₅H₃₁N₅O₆ 497.23 m/z, found [M+H]+ 498.25 m/z and (1R,3S)-3-(3-((*)-4-formyl-5-hydroxy-7-methoxy-2,3-dihydro-1H-indene-2-carboxamido)-1H-pyrazol-5-yl)cyclopentyl 2-methylpyrazolidine-1-carboxylate (Compound 263, 15.0 mg, 6.11% yield) as a white solid. 1H NMR (400 MHz, DMSO-d6) δ 12.08 (s, 1H), 11.24 (s, 1H), 10.43 (s, 1H), 10.07-10.06 (m, 1H), 6.39-6.34 (m, 2H), 5.04-5.03 (m, 1H), 3.84 (s, 3H), 3.43-3.38 (m, 4H), 3.30-3.23 (m, 1H), 3.17-3.13 (m, 1H), 2.98-2.92 (m, 1H), 2.87-2.80 (m, 3H), 2.42-2.35 (m, 4H), 2.09-2.01 (m, 3H), 1.86-1.74 (m, 4H). MS (ESI) calcd. for C₂₅H₃₁N₅O₆ 497.23 m/z, found [M+H]⁺ 498.20 m/z. * Denotes a stereocenter with an undetermined absolute stereochemistry of a single isomer.

### Example 23: Preparation of Compounds 264 and 265

### Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate (Compound 264) and (1R,3S)-3-(3-((*)-4-formyl-5-hydroxy-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate (Compound 265)

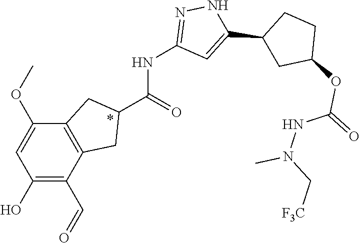 [SMILES: COc1cc(O)c(C=O)c2c1CC(C(=O)Nc1cc([C@H]3CC[C@@H](OC(=O)NN(C)CC(F)(F)F)C3)[nH]n1)C2]

### Synthetic Route

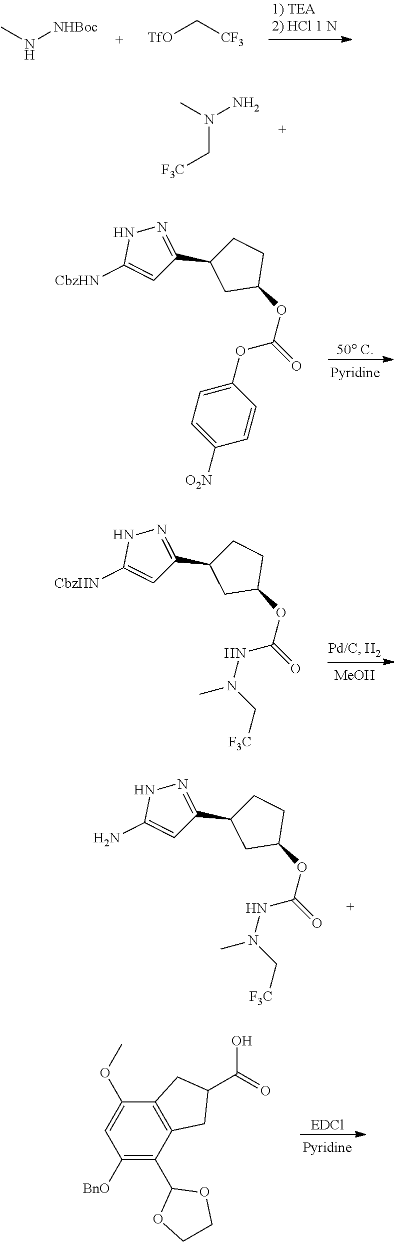

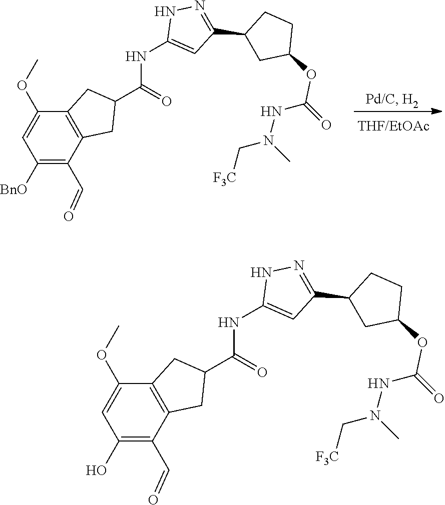 [SMILES: COc1cc(OCc2ccccc2)c(C=O)c2c1CC(C(=O)Nc1cc([C@H]3CC[C@@H](OC(=O)NN(C)CC(F)(F)F)C3)n[nH]1)C2 ; COc1cc(O)c(C=O)c2c1CC(C(=O)Nc1cc([C@H]3CC[C@@H](OC(=O)NN(C)CC(F)(F)F)C3)n[nH]1)C2]

### Step 1: Synthesis of 1-methyl-1-(2,2,2-trifluoroethyl)hydrazine

To a stirred solution of 2,2,2-trifluoroethyl trifluoromethanesulfonate (12 g, 51.702 mmol, 1 equiv.) in anhydrous diethyl ether (100 mL) was added N′-methyltert-butoxycarbohydrazide (7.56 g, 51.702 mmol, 1 equiv.) and Et₃N (35.93 mL, 258.510 mmol, 5 equiv.) at room temperature. The reaction mixture was stirred at room temperature for 10 h. The resulting solution was diluted with water and extracted with EtOAc (3×300 mL). The combined organic extracts were washed with H₂O (3×300 mL), dried over anhydrous Na₂SO₄, filtered, and concentrated under reduced pressure. To the crude material was added HCl in EtOAc (1M, 60 mL) and stirred at room temperature for 1 h. The resulting mixture was filtered, the filter cake was washed with EtOAc (3×100 mL) and the filtrate was concentrated under reduced pressure resulting 1-methyl-1-(2,2,2-trifluoroethyl)hydrazine (6 g, 90% yield). The crude product was used in the next step directly without further purification.

### Step 2: Synthesis of (1R,3S)-3-(3-(((benzyloxy)carbonyl)amino)-1H-pyrazol-5-yl)cyclopentyl 2-methyl-2-(2,2,2-trifluoroethyl)hydrazine-1-carboxylate

To a stirred solution of 1-methyl-1-(2,2,2-trifluoroethyl)hydrazine (6 g, 46.839 mmol, 4 equiv.) in anhydrous pyridine (100 mL) was added Intermediate C (5.46 g, 11.710 mmol, 1 equiv.) at room temperature. The reaction mixture was stirred at 50° C. for 4 h. The resulting mixture was cooled to room temperature, diluted with EtOAc (200 mL), washed with 0.5 M NaOH (2×100 mL), followed by with brine (2×100 mL), dried over anhydrous Na₂SO₄, filtered, and concentrated under reduced pressure. The resulting crude material was purified by reversed-phase flash chromatography with 10% to 70% MeCN/H₂O resulting in (1R,3S)-3-(3-(((benzyloxy)carbonyl)amino)-1H-pyrazol-5-yl)cyclopentyl 2-methyl-2-(2,2,2-trifluoroethyl)hydrazine-1-carboxylate (0.6 g, 11% yield).

### Step 3: Synthesis of (1R,3S)-3-(3-amino-1H-pyrazol-5-yl)cyclopentyl 2-methyl-2-(2,2,2-trifluoroethyl)hydrazine-1-carboxylate

To a solution of (1R,3S)-3-(3-(((benzyloxy)carbonyl)amino)-1H-pyrazol-5-yl)cyclopentyl 2-methyl-2-(2,2,2-trifluoroethyl)hydrazine-1-carboxylate (600 mg, 1.317 mmol, 1 equiv.) in MeOH (10 mL) was added 10% Pd/C (300 mg). The mixture was stirred for 2 h under hydrogen atmosphere. The reaction mixture was filtered through a Celite pad and concentrated under reduced pressure resulting in (1R,3S)-3-(3-amino-1H-pyrazol-5-yl)cyclopentyl 2-methyl-2-(2,2,2-trifluoroethyl)hydrazine-1-carboxylate (0.36 g, 85% yield). The crude product was used in the next step directly without further purification.

### Step 4: Synthesis of (1R,3S)-3-(3-((*)-5-(benzyloxy)-4-formyl-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate

To a stirred solution of (1R,3S)-3-(3-amino-1H-pyrazol-5-yl)cyclopentyl 2-methyl-2-(2,2,2-trifluoroethyl)hydrazine-1-carboxylate (300 mg, 0.810 mmol, 1 equiv.) in anhydrous pyridine (5 mL) was added 1-{[(1R,3S)-3-(5-amino-2H-pyrazol-3-yl)cyclopentyl]oxy}-N′-methyl-N′-(2,2,2-trifluoroethyl)formohydrazide (260.23 mg, 0.810 mmol, 1 equiv.) and EDCI (188.61 mg, 1.215 mmol, 1.5 equiv.). The reaction mixture was stirred at room temperature for 1 h. The resulting mixture was diluted with water and extracted with EtOAc (3×20 mL). The combined organic layers were washed with brine (3×20 mL), dried over anhydrous Na₂SO₄, filtered, and concentrated under reduced pressure. The crude material was purified by reversed-phase flash chromatography with the following conditions: H₂O (0.05% TFA) in ACN, 10% to 90% to afford (1R,3S)-3-(3-((*)-5-(benzyloxy)-4-formyl-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate (0.15 g, 27% yield).

### Step 5: Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate and (1R,3S)-3-(3-((*)-4-formyl-5-hydroxy-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate

To a solution of (1R,3S)-3-(3-((S)-5-(benzyloxy)-4-formyl-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate (150 mg, 0.238 mmol, 1 equiv.) in THF (2 mL) and EtOAc (2 mL) was added 10% Pd/C (150 mg). The mixture was stirred for 1 h under hydrogen atmosphere, filtered through a Celite pad and concentrated under reduced pressure. The crude product was purified by Prep-Chiral-HPLC (MeOH:DCM=1:1) to afford (1R,3S)-3-(3-((*)-4-formyl-5-hydroxy-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate (Compound 264, 9.8 mg, 7% yield). 1H NMR (400 MHz, DMSO-d6) δ 12.07 (s, 1H), 11.26 (s, 1H), 10.44 (s, 1H), 10.06 (s, 1H), 8.79 (s, 1H), 6.39 (s, 1H), 6.32 (s, 1H), 5.09-4.96 (m, 1H), 3.83 (s, 3H), 3.55-3.41 (m, 4H), 3.30-3.21 (m, 1H), 3.11-3.01 (m, 1H), 3.00-2.91 (m, 1H), 2.88-2.78 (m, 1H), 2.63 (m, 3H), 2.48-2.43 (m, 1H), 2.06-1.95 (m, 1H), 1.95-1.83 (m, 1H), 1.79-1.53 (m, 3H). 19F NMR (376 MHz, DMSO) δ −69.17. MS (ESI) calcd. for C₂₄H₂₈F₃N₅O₆ 539.20 m/z, found [M+H]⁺ 540.25 m/z and (1R,3S)-3-(3-((*)-4-formyl-5-hydroxy-7-methoxy-2,3-dihydro-1H-indene-2-carboxamido)-1H-pyrazol-5-yl)cyclopentyl 2-methyl-2-(2,2,2-trifluoroethyl)hydrazine-1-carboxylate (Compound 265, 19.4 mg, 15% yield). 1H NMR (400 MHz, DMSO-d6) δ 12.06 (s, 1H), 11.25 (s, 1H), 10.43 (s, 1H), 10.06 (s, 1H), 8.78 (s, 1H), 6.39 (s, 1H), 6.32 (s, 1H), 5.09-4.96 (m, 1H), 3.84 (s, 3H), 3.49-3.41 (m, 4H), 3.30-3.21 (m, 1H), 3.11-3.01 (m, 1H), 3.00-2.91 (m, 1H), 2.88-2.78 (m, 1H), 2.63 (m, 3H), 2.48-2.43 (m, 1H), 2.06-1.95 (m, 1H), 1.95-1.83 (m, 1H), 1.79-1.53 (m, 3H). MS (ESI) calcd. for C₂₄H₂₈F₃N₅O₆ 539.20 m/z, found [M+H]⁺ 540.25 m/z. * Denotes a stereocenter with an undetermined absolute stereochemistry of a single isomer.

### Example 24: Preparation of Compounds 266 and 267

### Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2-methyltetrahydropyridazine-1(2H)-carboxylate (Compound 266) and (1R,3S)-3-(3-((*)-4-formyl-5-hydroxy-7-methoxy-2,3-dihydro-1H-indene-2-carboxamido)-1H-pyrazol-5-yl)cyclopentyl 2-methyltetrahydropyridazine-1(2H)-carboxylate (Compound 267)

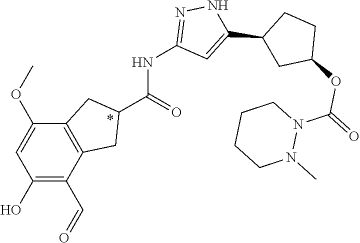 [SMILES: COc1cc(O)c(C=O)c2c1CC(C(=O)Nc1cc([C@H]3CC[C@@H](OC(=O)N4CCCCN4C)C3)[nH]n1)C2]

### Synthetic Route

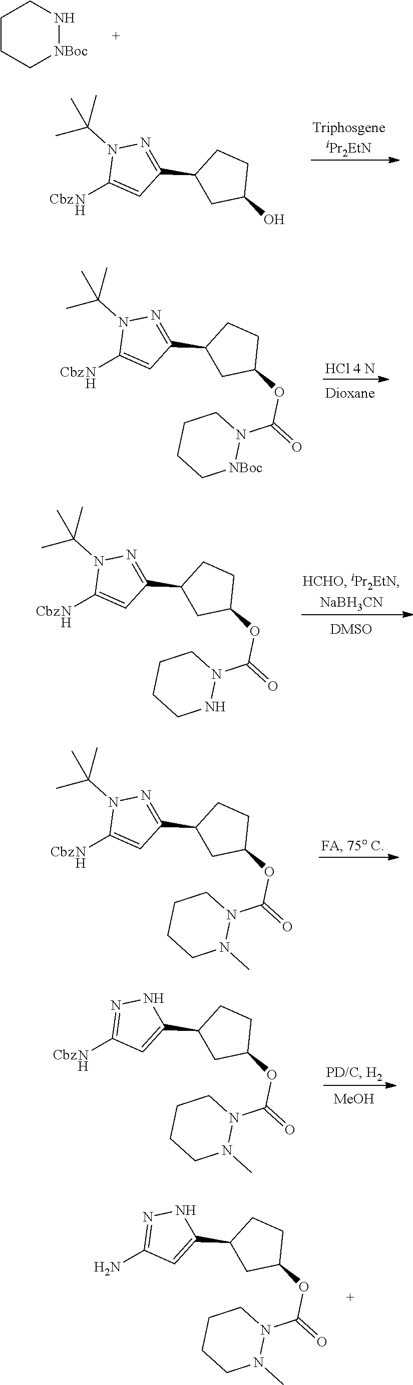

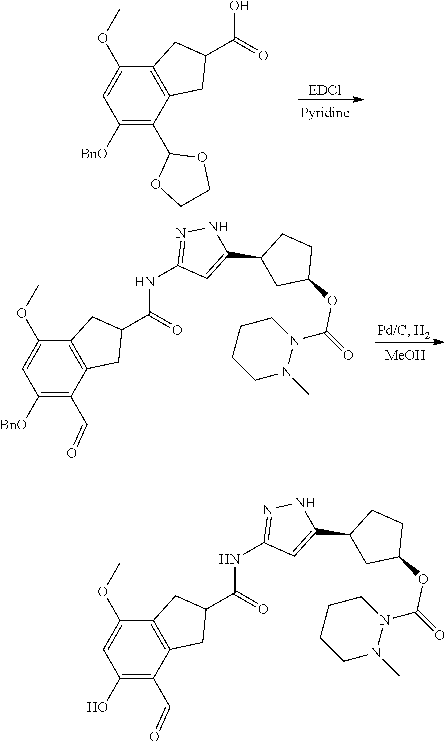

### Step 1: Synthesis of 1-((1R,3S)-3-(5-(((benzyloxy)carbonyl)amino)-1-(tert-butyl)-1H-pyrazol-3-yl)cyclopentyl) 2-(tert-butyl) tetrahydropyridazine-1,2-dicarboxylate

A stirred solution of benzyl N-{2-tert-butyl-5-[(1S,3R)-3-hydroxycyclopentyl]pyrazol-3-yl}carbamate (3 g, 8.393 mmol, 1.00 equiv.) in DCM (50 mL) was treated with triphosgene (2.5 g, 8.4 mmol, 1 equiv.) at room temperature followed by the addition of DIEA (6.51 g, 50.358 mmol, 6 equiv.). The resulting mixture was stirred at room temperature for 1 h under nitrogen atmosphere and concentrated under reduced pressure. To a stirred solution of tert-butyl 1,2-diazinane-1-carboxylate (1.56 g, 8.393 mmol, 1 equiv.) and DIEA (6.51 g, 50.358 mmol, 6 equiv.) in THF (50 mL) was added the above residue at room temperature and stirred at room temperature for 2 h. The reaction was quenched with water and extracted with DCM (2×50 mL), the combined organic extracts were washed with brine (2×50 mL), dried over anhydrous Na₂SO₄, and concentrated under reduced atmosphere. The crude material was purified by C18 reverse phase chromatography, 5 to 50% MeCN/H₂O to afford 1-((1R,3S)-3-(5-(((benzyloxy)carbonyl)amino)-1-(tert-butyl)-1H-pyrazol-3-yl)cyclopentyl) 2-(tert-butyl) tetrahydropyridazine-1,2-dicarboxylate (3.75 g, 78%) as a yellow oil.

### Step 2: Synthesis of (1R,3S)-3-(5-(((benzyloxy)carbonyl)amino)-1-(tert-butyl)-1H-pyrazol-3-yl)cyclopentyl tetrahydropyridazine-1(2H)-carboxylate

1-((1R,3S)-3-(5-(((benzyloxy)carbonyl)amino)-1-(tert-butyl)-1H-pyrazol-3-yl)cyclopentyl) 2-(tert-butyl) tetrahydropyridazine-1,2-dicarboxylate (3.7 g, 6.495 mmol, 1 equiv.) was suspended in a solution of HCl (4.0 M in 1,4-dioxane, 20 mL) amd EtOAc (40 mL) and stirred at room temperature 1 h. The resulting mixture was concentrated under reduced pressure and washed with 100 mL of hexane resulting in (1R,3S)-3-(5-(((benzyloxy)carbonyl)amino)-1-(tert-butyl)-1H-pyrazol-3-yl)cyclopentyl tetrahydropyridazine-1(2H)-carboxylate (3 g, 98% yield) as a yellow solid. The crude material was used without any further purification.

### Step 3: Synthesis of (1R,3S)-3-(5-(((benzyloxy)carbonyl)amino)-1-(tert-butyl)-1H-pyrazol-3-yl)cyclopentyl 2-methyltetrahydropyridazine-1(2H)-carboxylate

A solution of (1R,3S)-3-(5-(((benzyloxy)carbonyl)amino)-1-(tert-butyl)-1H-pyrazol-3-yl)cyclopentyl tetrahydropyridazine-1(2H)-carboxylate (2.74 g, 5.835 mmol, 1 equiv.) in DMSO (50 mL) was basified to pH ˜8 with DIEA at room temperature followed by the dropwise addition of formaldehyde (1.31 mL, 17.505 mmol, 3 equiv., 40% in H₂O). The mixture was acidified to pH ˜4 with acetic acid, stirred for an additional 3 h followed by the portion-wise addition of NaBH₃CN (1.10 g, 17.505 mmol, 3 equiv.) and left to stir overnight at room temperature. The reaction was quenched with water and the mixture was extracted with EtOAc (2×150 mL), the combined organic extracts were washed with brine (2×150 mL), dried over anhydrous Na₂SO₄, and concentrated under reduced atmosphere. The crude material was purified by C18 reverse phase chromatography, 5 to 60% MeCN/H₂O to afford (1R,3S)-3-(5-(((benzyloxy)carbonyl)amino)-1-(tert-butyl)-1H-pyrazol-3-yl)cyclopentyl 2-methyltetrahydropyridazine-1(2H)-carboxylate (1.39 g, 49% yield) as a yellow oil.

### Step 4: Synthesis of (1R,3S)-3-(3-(((benzyloxy)carbonyl)amino)-1H-pyrazol-5-yl)cyclopentyl 2-methyltetrahydropyridazine-1(2H)-carboxylate

A solution of (1R,3S)-3-(5-(((benzyloxy)carbonyl)amino)-1-(tert-butyl)-1H-pyrazol-3-yl)cyclopentyl 2-methyltetrahydropyridazine-1(2H)-carboxylate (1.39 g, 3.970 mmol, 1 equiv.) in formic acid (30 mL) was stirred at 75° C. for 10 h. The resulting mixture was cooled to room temperature and concentrated under vacuum. The crude material was purified by C18 reverse phase chromatography, 5 to 46% MeCN/H₂O to afford (1R,3S)-3-(3-(((benzyloxy)carbonyl)amino)-1H-pyrazol-5-yl)cyclopentyl 2-methyltetrahydropyridazine-1(2H)-carboxylate (1.1 g, 90% yield) as a white solid.

### Step 5: Synthesis of (1R,3S)-3-(3-amino-1H-pyrazol-5-yl)cyclopentyl 2-methyltetrahydropyridazine-1(2H)-carboxylate

To a stirred solution of (1R,3S)-3-(3-(((benzyloxy)carbonyl)amino)-1H-pyrazol-5-yl)cyclopentyl 2-methyltetrahydropyridazine-1(2H)-carboxylate (1.1 g, 3.251 mmol, 1 equiv.) in MeOH (20 mL) was added 10% Pd/C (1 g). The mixture was maintained under hydrogen atmosphere at room temperature overnight. The resulting mixture was filtered, the filter cake was washed with DCM (2×20 mL). The filtrate was concentrated under reduced pressure to afford (1R,3S)-3-(3-amino-1H-pyrazol-5-yl)cyclopentyl 2-methyltetrahydropyridazine-1(2H)-carboxylate (0.7 g, 93% yield) as a white solid. The crude material was used without any further purification.

### Step 6: Synthesis of (1R,3S)-3-(3-(5-(benzyloxy)-4-formyl-7-methoxy-2,3-dihydro-1H-indene-2-carboxamido)-1H-pyrazol-5-yl)cyclopentyl 2-methyltetrahydropyridazine-1(2H)-carboxylate

To a stirred solution of (1R,3S)-3-(3-amino-1H-pyrazol-5-yl)cyclopentyl 2-methyltetrahydropyridazine-1(2H)-carboxylate (396.02 mg, 1.350 mmol, 1 equiv.) and 5-(benzyloxy)-4-(1,3-dioxolan-2-yl)-7-methoxy-2,3-dihydro-1H-indene-2-carboxylic acid (500 mg, 1.350 mmol, 1.00 equiv.) in pyridine (5 mL) was added EDCI (314.35 mg, 2.025 mmol, 1.5 equiv.) at room temperature. The resulting mixture was stirred at room temperature for 1 h. The reaction was quenched with water and then the mixture was extracted with EtOAc (2×15 mL). The combined organic extracts were washed with brine (2×15 mL), dried over anhydrous Na₂SO₄, and concentrated under vacuum. The crude material was purified by C18 reverse phase chromatography, 5% to 60% MeCN/H₂O to afford (1R,3S)-3-(3-(5-(benzyloxy)-4-formyl-7-methoxy-2,3-dihydro-1H-indene-2-carboxamido)-1H-pyrazol-5-yl)cyclopentyl 2-methyltetrahydropyridazine-1(2H)-carboxylate (420 mg, 52% yield) as a yellow solid.

### Step 7: Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2-methyltetrahydropyridazine-1(2H)-carboxylate and (1R,3S)-3-(3-((*)-4-formyl-5-hydroxy-7-methoxy-2,3-dihydro-H-indene-2-carboxamido)-1H-pyrazol-5-yl)cyclopentyl 2-methyltetrahydropyridazine-1(2H)-carboxylate

To a stirred solution of (1R,3S)-3-(3-(5-(benzyloxy)-4-formyl-7-methoxy-2,3-dihydro-1H-indene-2-carboxamido)-1H-pyrazol-5-yl)cyclopentyl 2-methyltetrahydropyridazine-1(2H)-carboxylate (400 mg, 0.665 6 mmol, 1 equiv.) in MeOH (10 mL) was added 10% Pd/C (400 mg). The mixture was maintained under hydrogen atmosphere at room temperature for 1 h, the resulting mixture was filtered, the filter cake was washed with DCM (2×20 mL) and filtrate was concentrated under reduced pressure. The crude material was purified by C18 reverse phase chromatography, 5% to 41% MeCN/H₂O to afford (1R,3S)-3-(3-(4-formyl-5-hydroxy-7-methoxy-2,3-dihydro-1H-indene-2-carboxamido)-1H-pyrazol-5-yl)cyclopentyl 2-methyltetrahydropyridazine-1(2H)-carboxylate (160 mg). The product separated by chiral-SFC to afford (1R,3S)-3-(3-((*)-4-formyl-5-hydroxy-7-methoxy-2,3-dihydro-1H-indene-2-carboxamido)-1H-pyrazol-5-yl)cyclopentyl 2-methyltetrahydropyridazine-1(2H)-carboxylate (Compound 266, 13 mg, 3.82% yield) as a white solid. MS (ESI) calcd. for C₂₆H₃₃N₅O₆ 511.24 m/z, found [M+H]⁺ 512.20 m/z. 1H NMR (400 MHz, DMSO-d6) δ 12.09 (br s, 1H), 11.23 (s, 1H), 10.42 (s, 1H), 10.06 (s, 1H), 6.39 (s, 1H), 6.35 (s, 1H), 5.08-5.03 (m, 1H), 3.83 (s, 3H), 3.49-3.37 (m, 4H), 3.28-3.24 (m, 2H), 2.98-2.80 (m, 4H), 2.57 (s, 3H), 2.37-2.28 (m, 1H), 2.11-2.04 (m, 1H), 1.84-1.80 (m, 2H), 1.73-1.63 (m, 2H), 1.56-1.52 (m, 4H) and (1R,3S)-3-(3-((*)-4-formyl-5-hydroxy-7-methoxy-2,3-dihydro-1H-indene-2-carboxamido)-1H-pyrazol-5-yl)cyclopentyl 2-methyltetrahydropyridazine-1(2H)-carboxylate (Compound 267, 13.5 mg, 3.97% yield) as a white solid. MS (ESI) calcd. for C₂₆H₃₃N₅O₆ 511.24 m/z, found [M+H]⁺ 512.20 m/z. 1H NMR (400 MHz, DMSO-d6) δ 12.09 (br s, 1H), 11.23 (s, 1H), 10.42 (s, 1H), 10.06 (s, 1H), 6.39 (s, 1H), 6.35 (s, 1H), 5.08-5.03 (m, 1H), 3.83 (s, 3H), 3.46-3.40 (m, 4H), 3.28-3.25 (m, 2H), 2.94-2.83 (m, 4H), 2.57 (s, 3H), 2.35-2.29 (m, 1H), 2.10-2.06 (m, 1H), 1.84-1.79 (m, 2H), 1.72-1.66 (m, 2H), 1.56-1.51 (m, 4H). * Denotes a stereocenter with an undetermined absolute stereochemistry of a single isomer.

### Example 25: Preparation of Compounds 268 and 269

### Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 2,2-dimethylhydrazine-1-carboxylate (Compound 268) and (1R,3S)-3-(3-((*)-4-formyl-5-hydroxy-7-methoxy-2,3-dihydro-H-indene-2-carboxamido)-1H-pyrazol-5-yl)cyclopentyl 2,2-dimethylhydrazine-1-carboxylate (Compound 269)

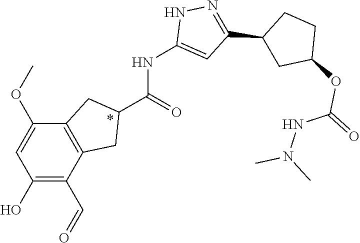 [SMILES: COc1cc(O)c(C=O)c2c1CC(C(=O)Nc1cc([C@H]3CC[C@@H](OC(=O)NN(C)C)C3)n[nH]1)C2]

### Synthetic Route

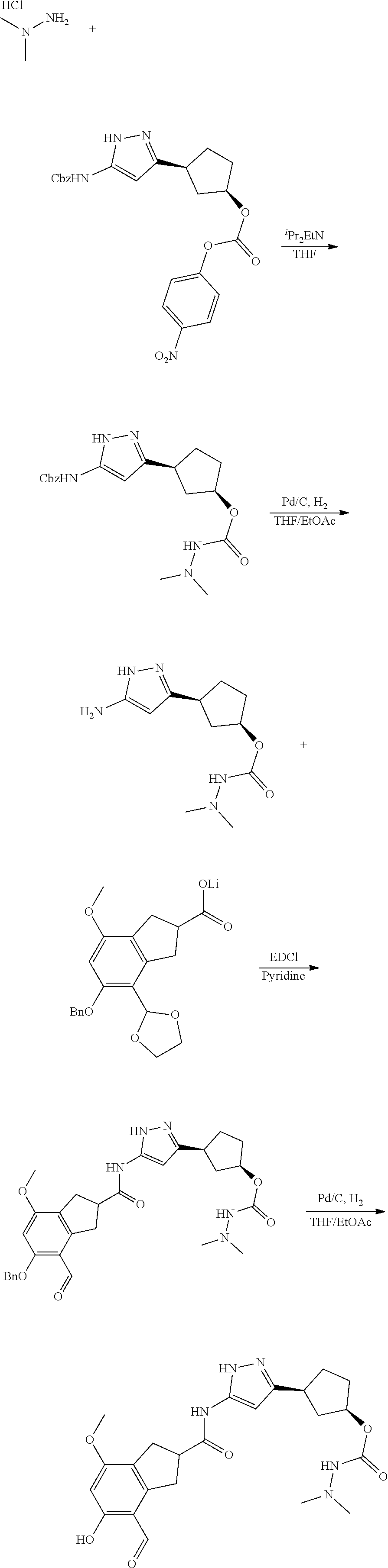

### Step 1: Synthesis of (1R,3S)-3-(3-(((benzyloxy)carbonyl)amino)-1H-pyrazol-5-yl)cyclopentyl 2,2-dimethylhydrazine-1-carboxylate

To a stirred solution of intermediate C (2.5 g, 5.360 mmol, 1 equiv.) and 1,1-dimethylhydrazine hydrochloride (1.04 g, 10.720 mmol, 2 equiv.) in THF (25 mL) was added DIEA (5.54 g, 42.880 mmol, 8 equiv.) dropwise at room temperature. The resulting mixture was stirred at room temperature for 2 h. The solvent was removed in vacuo. The residue was diluted with EtOAc (400 mL) and washed with 0.5 M NaOH (2×100 mL) and brine (50 mL). The recovered organic later was dried over anhydrous Na₂SO₄, filtered and concentrated under reduced pressure. The residue was purified by reversed-phase flash chromatography with MeCN in Water (0.1% TFA) to afford (1R,3S)-3-(3-(((benzyloxy)carbonyl)amino)-1H-pyrazol-5-yl)cyclopentyl 2,2-dimethylhydrazine-1-carboxylate (1.1 g, 52.97% yield) as a yellow oil.

### Step 2: Synthesis of (1R,3S)-3-(3-amino-1H-pyrazol-5-yl)cyclopentyl 2,2-dimethylhydrazine-1-carboxylate

To a stirred solution of (1R,3S)-3-(3-(((benzyloxy)carbonyl)amino)-1H-pyrazol-5-yl)cyclopentyl 2,2-dimethylhydrazine-1-carboxylate (1.0 g, 2.581 mmol, 1 equiv.) in EtOAc (5 mL) and THF (5 mL) was added 10% Pd/C (500 mg) at room temperature. The resulting mixture was stirred at room temperature for 2 h under hydrogen atmosphere. The resulting mixture was filtered, the filter cake was washed with EtOAc (4×50 mL) and the filtrate was concentrated under reduced pressure to afford (1R,3S)-3-(3-amino-1H-pyrazol-5-yl)cyclopentyl 2,2-dimethylhydrazine-1-carboxylate (480 mg, 73.42% yield) as a yellow oil. The crude material was used without any further purification.

### Step 3: Synthesis of (1R,3S)-3-(3-(5-(benzyloxy)-4-formyl-7-methoxy-2,3-dihydro-H-indene-2-carboxamido)-1H-pyrazol-5-yl)cyclopentyl 2,2-dimethylhydrazine-1-carboxylate

To a stirred solution of (1R,3S)-3-(3-amino-1H-pyrazol-5-yl)cyclopentyl 2,2-dimethylhydrazine-1-carboxylate (400 mg, 1.579 mmol, 1 equiv.) and lithium 5-(benzyloxy)-4-(1,3-dioxolan-2-yl)-7-methoxy-2,3-dihydro-1H-indene-2-carboxylate (594 mg, 1.578 mmol, 1.00 equiv.) in pyridine (5 mL) was added EDCI (368 mg, 2.370 mmol, 1.50 equiv.). The resulting mixture was stirred at room temperature for 3 h. The solvent was removed in vacuo. The resulting residue was diluted with EtOAc (100 mL), washed with water (3×50 mL), brine (30 mL), dried over anhydrous Na₂SO₄, filtered and concentrated under reduced pressure. The crude material was purified by reversed-phase flash chromatography with MeCN in Water (0.1% TFA) to afford (1R,3S)-3-(3-(5-(benzyloxy)-4-formyl-7-methoxy-2,3-dihydro-1H-indene-2-carboxamido)-1H-pyrazol-5-yl)cyclopentyl 2,2-dimethylhydrazine-1-carboxylate (120 mg, 13.53% yield) as a white solid.

### Step 4: Synthesis of (1R,3S)-3-(3-((*)-4-formyl-5-hydroxy-7-methoxy-2,3-dihydro-H-indene-2-carboxamido)-1H-pyrazol-5-yl)cyclopentyl 2,2-dimethylhydrazine-1-carboxylate and (1R,3S)-3-(3-((*)-4-formyl-5-hydroxy-7-methoxy-2,3-dihydro-1H-indene-2-carboxamido)-1H-pyrazol-5-yl)cyclopentyl 2,2-dimethylhydrazine-1-carboxylate

To a stirred solution of (1R,3S)-3-(3-(5-(benzyloxy)-4-formyl-7-methoxy-2,3-dihydro-1H-indene-2-carboxamido)-1H-pyrazol-5-yl)cyclopentyl 2,2-dimethylhydrazine-1-carboxylate (100 mg, 0.178 mmol, 1 equiv.) in EtOAc (3 mL) and THF (3 mL) was added 10% Pd/C (50 mg) at room temperature under air atmosphere. The resulting mixture was stirred at room temperature for 2 h under hydrogen atmosphere. The resulting mixture was filtered, the filter cake was washed with EtOAc (3×10 mL). The filtrate was concentrated under reduced pressure. The residue was purified by reversed-phase flash chromatography with MeCN in Water (0.1% TFA) to afford (1R,3S)-3-(3-(4-formyl-5-hydroxy-7-methoxy-2,3-dihydro-1H-indene-2-carboxamido)-1H-pyrazol-5-yl)cyclopentyl 2,2-dimethylhydrazine-1-carboxylate (60 mg) as a yellow solid. Then (1R,3S)-3-(3-(4-formyl-5-hydroxy-7-methoxy-2,3-dihydro-1H-indene-2-carboxamido)-1H-pyrazol-5-yl)cyclopentyl 2,2-dimethylhydrazine-1-carboxylate was separated by Chiral-HPLC with Mobile Phase A: MTBE (0.1% FA)—HPLC, Mobile Phase B: MeOH:DCM=1:1 to afford (1R,3S)-3-(3-((*)-4-formyl-5-hydroxy-7-methoxy-2,3-dihydro-1H-indene-2-carboxamido)-1H-pyrazol-5-yl)cyclopentyl 2,2-dimethylhydrazine-1-carboxylate (Compound 268, 2.8 mg, 3.34% yield) as a white solid. 1H NMR (400 MHz, DMSO-d6) δ 12.06 (s, 1H), 11.23 (s, 1H), 10.42 (s, 1H), 10.06 (s, 1H), 8.15 (s, 1H), 6.38-6.31 (m, 1H), 5.12-4.92 (m, 1H), 3.83 (s, 3H), 3.49-3.40 (m, 2H), 3.37-3.24 (m, 1H), 3.24-3.17 (m, 1H), 3.16-3.04 (m, 1H), 3.04-2.92 (m, 1H), 2.92-2.80 (m, 1H), 2.43 (s, 6H), 2.13-1.95 (m, 1H), 1.94-1.81 (m, 1H), 1.80-1.49 (m, 3H). MS(ESI) calcd for C₂₃H₂₉N₅O₆ 471.21 m/z, found [M+H]⁺ 472.15 m/z and (1R,3S)-3-(3-((*)-4-formyl-5-hydroxy-7-methoxy-2,3-dihydro-1H-indene-2-carboxamido)-1H-pyrazol-5-yl)cyclopentyl 2,2-dimethylhydrazine-1-carboxylate (Compound 269, 2.3 mg, 2.74% yield) as a white solid. 1H NMR (400 MHz, DMSO-d6) δ 12.06 (s, 1H), 11.21 (s, 1H), 10.41 (s, 1H), 10.06 (s, 1H), 8.15 (s, 1H), 6.38-6.32 (m, 2H), 5.12-4.99 (m, 1H), 3.83 (s, 3H), 3.48-3.39 (m, 2H), 3.27-3.21 (m, 2H), 3.17-3.04 (m, 1H), 3.04-2.98 (m, 1H), 2.95-2.79 (m, 1H), 2.41 (s, 6H), 2.17-1.95 (m, 1H), 1.94-1.81 (m, 1H), 1.80-1.49 (m, 3H). MS(ESI) calcd for C₂₃H₂₉N₅O₆ 471.21 m/z, found [M+H]⁺ 472.15 m/z. * Denotes a stereocenter with an undetermined absolute stereochemistry of a single isomer.

### Example 26: Preparation of Compounds 270 and 271

### Synthesis of (1R,3S)-3-(3-((S)-4-formyl-5-hydroxy-7-methoxy-2,3-dihydro-1H-indene-2-carboxamido)-1H-pyrazol-5-yl)cyclobutyl 1,2,2-trimethylhydrazine-1-carboxylate (Compound 270) and (1S,3S)-3-(3-((R)-4-formyl-5-hydroxy-7-methoxy-2,3-dihydro-1H-indene-2-carboxamido)-1H-pyrazol-5-yl)cyclobutyl 1,2,2-trimethylhydrazine-1-carboxylate (Compound 271)

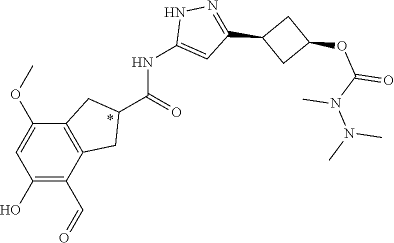 [SMILES: COc1cc(O)c(C=O)c2c1CC(C(=O)Nc1cc([C@H]3C[C@@H](OC(=O)N(C)N(C)C)C3)n[nH]1)C2]

### Synthetic Route

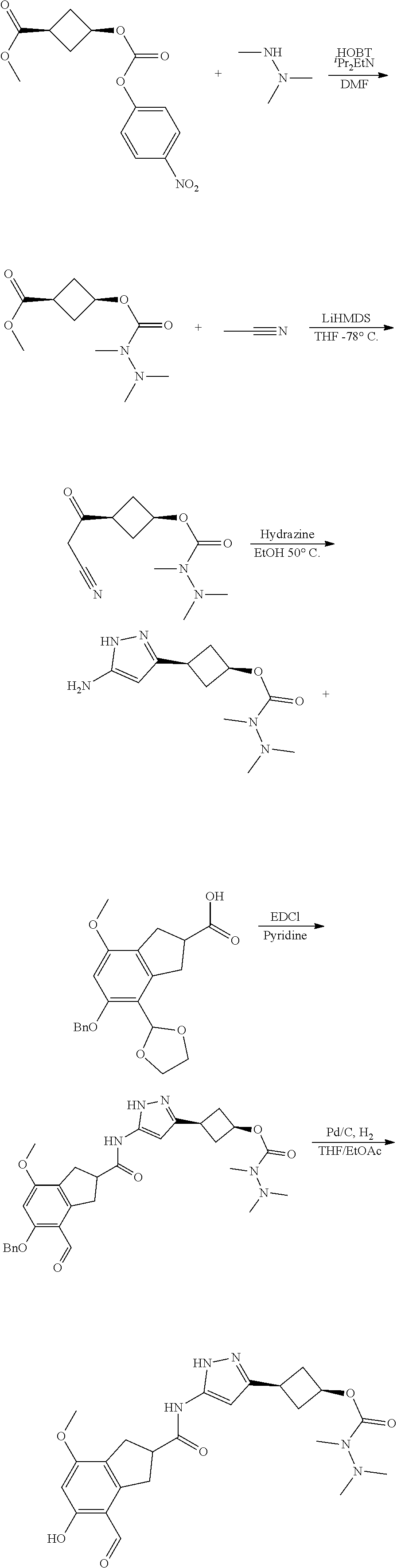

### Step 1: Synthesis of (1s,3s)-3-(methoxycarbonyl)cyclobutyl 1,2,2-trimethylhydrazine-1-carboxylate

To a stirred solution of methyl (1s,3s)-3-(((4-nitrophenoxy)carbonyl)oxy)cyclobutane-1-carboxylate (4 g, 13.548 mmol, 1 equiv.) in anhydrous DMF (40 mL) was added 1,1,2-trimethylhydrazine (2.01 g, 27.096 mmol, 2 equiv.) and HOBT (3.66 g, 27.096 mmol, 2 equiv.) followed DIEA (14.16 mL, 81.288 mmol, 6 equiv.) at room temperature. The reaction mixture was stirred at room temperature for 4 h. The resulting mixture was diluted with water and extracted with EtOAc (3×100 mL). The combined organic layers were washed with brine (3×100 mL), dried over anhydrous Na₂SO₄, filtered, and concentrated under reduced pressure. The residue was purified by silica gel column chromatography, eluted with PE/EtOAc (1/1) to afford methyl (1s,3s)-3-[(N,N′,N′-trimethylhydrazinecarbonyl)oxy]cyclobutane-1-carboxylate (2 g, 64% yield).

### Step 2: Synthesis of (1s,3s)-3-(2-cyanoacetyl)cyclobutyl 1,2,2-trimethylhydrazine-1-carboxylate

In a 250-mL round bottom flask, to a solution of methyl (1s,3s)-3-[(N,N′,N′-trimethylhydrazinecarbonyl)oxy]cyclobutane-1-carboxylate (2 g, 8.686 mmol, 1 equiv.) and acetonitrile (1.07 g, 26.058 mmol, 3 equiv.) in THF (10 mL) was added dropwise LiHMDS (1.0 M in THF) (4.36 mL, 26.058 mmol, 3 equiv.) at −78° C. under N₂ atmosphere. The reaction mixture was stirred at −78° C. for 40 mins. The reaction was warm to 0° C., quenched with water and then acidified to pH 1 with HCl (2M). The resulting mixture was extracted with EtOAc (3×50 mL). The combined organic layers were washed with brine (2×20 mL), dried over anhydrous Na₂SO₄, filtered, and concentrated under reduced pressure. The crude product mixture was used in the next step directly without further purification. This resulted in (1s,3s)-3-(2-cyanoacetyl)cyclobutyl 1,2,2-trimethylhydrazine-1-carboxylate (1.8 g, 86% yield).

### Step 3: Synthesis of (1s,3s)-3-(3-amino-1H-pyrazol-5-yl)cyclobutyl 1,2,2-trimethylhydrazine-1-carboxylate

To a stirred solution of (1s,3s)-3-(2-cyanoacetyl)cyclobutyl 1,2,2-trimethylhydrazine-1-carboxylate (1.8 g, 7.523 mmol, 1 equiv.) in anhydrous EtOH (30 mL) was added hydrazine (1.74 g, 54.330 mmol, 5 equiv.) at room temperature. The reaction mixture was stirred at 50° C. for 4 h. The resulting mixture cooled to room temperature, diluted with water and concentrated under reduced pressure. The residue was purified by reversed-phase flash chromatography with mobile phase, 10% to 20% MeCN/H₂O to afford (1s,3s)-3-(3-amino-1H-pyrazol-5-yl)cyclobutyl 1,2,2-trimethylhydrazine-1-carboxylate (1.6 g, 58% yield).

### Step 4: Synthesis of (1s,3s)-3-(3-(5-(benzyloxy)-4-formyl-7-methoxy-2,3-dihydro-1H-indene-2-carboxamido)-1H-pyrazol-5-yl)cyclobutyl 1,2,2-trimethylhydrazine-1-carboxylate

To a stirred solution of (1s,3s)-3-(3-amino-1H-pyrazol-5-yl)cyclobutyl 1,2,2-trimethylhydrazine-1-carboxylate (350 mg, 1.382 mmol, 1 equiv.) in anhydrous pyridine (10 mL) was added 5-(benzyloxy)-4-(1,3-dioxolan-2-yl)-7-methoxy-2,3-dihydro-1H-indene-2-carboxylic acid (511.79 mg, 1.382 mmol, 1 equiv.) and EDCI (321.76 mg, 2.073 mmol, 1.5 equiv.) at room temperature and stirred for 2 h. The resulting mixture was diluted with water and extracted with EtOAc (3×20 mL). The combined organic layers were washed with brine (3×20 mL), dried over anhydrous Na₂SO₄, filtered, and concentrated under reduced pressure. The crude material was purified by reversed-phase flash chromatography with 10% to 70% MeCN/H₂O with 0.05% TFA modifier to afford (1s,3s)-3-(3-(5-(benzyloxy)-4-formyl-7-methoxy-2,3-dihydro-1H-indene-2-carboxamido)-1H-pyrazol-5-yl)cyclobutyl 1,2,2-trimethylhydrazine-1-carboxylate (300 mg, 38% yield).

### Step 5: Synthesis of (1R,3S)-3-(3-((*)-4-formyl-5-hydroxy-7-methoxy-2,3-dihydro-1H-indene-2-carboxamido)-1H-pyrazol-5-yl)cyclobutyl 1,2,2-trimethylhydrazine-1-carboxylate and (1S,3S)-3-(3-((*)-4-formyl-5-hydroxy-7-methoxy-2,3-dihydro-1H-indene-2-carboxamido)-1H-pyrazol-5-yl)cyclobutyl 1,2,2-trimethylhydrazine-1-carboxylate

To a solution of (1s,3s)-3-(3-(5-(benzyloxy)-4-formyl-7-methoxy-2,3-dihydro-1H-indene-2-carboxamido)-1H-pyrazol-5-yl)cyclobutyl 1,2,2-trimethylhydrazine-1-carboxylate (100 mg, 0.178 mmol, 1 equiv.) in THF (2 mL) and EtOAc (2 mL) was added 10% Pd/C (100 mg) under hydrogen atmosphere. The mixture was maintained under hydrogen atmosphere at room temperature for 1 h under, followed by filtration through a Celite pad and concentrated under reduced pressure. The crude product was purified by Chiral-HPLC with the following conditions (MtBE (0.1% FA): (MeOH:DCM=1:1)=60:40) to afford (1R,3S)-3-(3-((*)-4-formyl-5-hydroxy-7-methoxy-2,3-dihydro-1H-indene-2-carboxamido)-1H-pyrazol-5-yl)cyclobutyl 1,2,2-trimethylhydrazine-1-carboxylate (Compound 270, 13.0 mg, 15% yield). 1H NMR (400 MHz, DMSO-d6) δ 12.16 (s, 1H), 11.24 (s, 1H), 10.44 (s, 1H), 10.07 (s, 1H), 6.39-6.38 (m, 2H), 4.85-4.81 (m, 1H), 3.84 (s, 3H), 3.52-3.39 (m, 2H), 3.30-3.24 (m, 1H), 3.12-3.03 (m, 1H), 3.03-2.91 (m, 1H), 2.90-2.80 (m, 4H), 2.73-2.64 (m, 2H), 2.49-2.46 (m, 6H), 2.17-2.06 (m, 2H). MS (ESI) calcd. for C₂₃H₂₉N₅O₆ 471.21 m/z, found [M+H]⁺ 472.25 m/z and (1S,3S)-3-(3-((*)-4-formyl-5-hydroxy-7-methoxy-2,3-dihydro-1H-indene-2-carboxamido)-1H-pyrazol-5-yl)cyclobutyl 1,2,2-trimethylhydrazine-1-carboxylate (Compound 271, 12.5 mg, 14% yield). 1H NMR (400 MHz, DMSO-d6) δ 12.16 (s, 1H), 11.24 (s, 1H), 10.44 (s, 1H), 10.07 (s, 1H), 6.39-6.37 (m, 2H), 4.85-4.81 (m, 1H), 3.84 (s, 3H), 3.51-3.41 (m, 2H), 3.30-3.24 (m, 1H), 3.12-3.03 (m, 1H), 3.01-2.93 (m, 1H), 2.90-2.81 (m, 4H), 2.73-2.64 (m, 2H), 2.49-2.46 (m, 6H), 2.17-2.06 (m, 2H). MS (ESI) calcd. for C₂₃H₂₉N₅O₆ 471.21 m/z, found [M+H]⁺ 472.25 m/z. * Denotes a stereocenter with an undetermined absolute stereochemistry of a single isomer.

### Example 27: Preparation of Compound 272

### Synthesis of (1R,3S)-3-(3-(2-(2-formyl-3-hydroxy-5-methoxyphenoxy)acetamido)-1H-pyrazol-5-yl)cyclopentyl 3-methyl-2,3-diazabicyclo[2.2.1]heptane-2-carboxylate

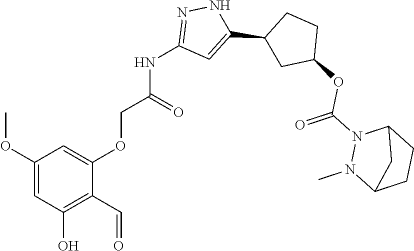 [SMILES: COc1cc(O)c(C=O)c(OCC(=O)Nc2cc([C@H]3CC[C@@H](OC(=O)N4C5CCC(C5)N4C)C3)[nH]n2)c1]

### Synthetic Route

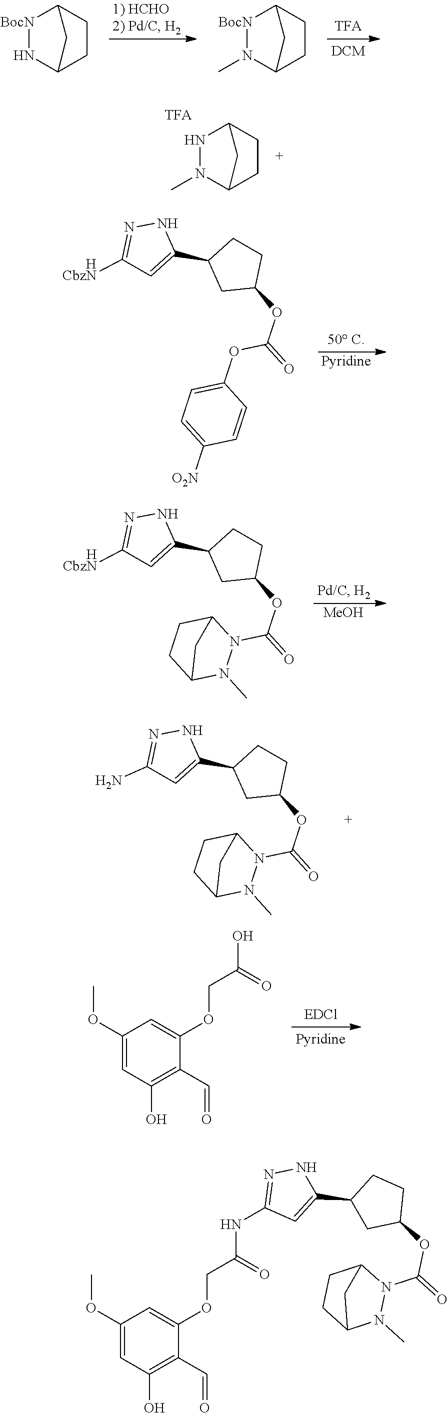

### Step 1: Synthesis of tert-butyl 3-methyl-2,3-diazabicyclo[2.2.1]heptane-2-carboxylate

To a stirred solution of tert-butyl 2,3-diazabicyclo[2.2.1]heptane-2-carboxylate (4 g, 20.175 mmol, 1 equiv.) in MeOH (40 mL) was added HCHO (1.8 mL, 24.210 mmol, 1.2 equiv., 40% in H₂O) dropwise at room temperature. The resulting mixture was stirred at room temperature for 3 h. To the above mixture was added 10% Pd/C (5 g). The mixture was maintained under hydrogen atmosphere at room temperature for 3 h. The resulting mixture was filtered, the filter cake was washed with MeOH (2×50 mL). The filtrate was concentrated under reduced pressure resulting in tert-butyl 3-methyl-2,3-diazabicyclo[2.2.1]heptane-2-carboxylate (3.9 g, 91% yield) as a colorless oil. The crude material was used without any further purification.

### Step 2: Synthesis of 2-methyl-2,3-diazabicyclo[2.2.1]heptane; 2,2,2-trifluoro-l13-ethan-1-one

A solution of tert-butyl 3-methyl-2,3-diazabicyclo[2.2.1]heptane-2-carboxylate (1.2 g, 5.653 mmol, 1 equiv.) in TFA (5 mL) and DCM (15 mL) was stirred at room temperature for 5 h. The mixture was concentrated under reduced pressure. This resulted in 2-methyl-2,3-diazabicyclo[2.2.1]heptane; 2,2,2-trifluoro-l13-ethan-1-one (0.90 g, 76% yield) as a light yellow oil. The crude material was used without any further purification.

### Step 3: Synthesis of (1R,3S)-3-(3-(((benzyloxy)carbonyl)amino)-1H-pyrazol-5-yl)cyclopentyl 3-methyl-2,3-diazabicyclo[2.2.1]heptane-2-carboxylate

A solution of 2-methyl-2,3-diazabicyclo[2.2.1]heptane; 2,2,2-trifluoro-l13-ethan-1-one (0.90 g, 4.288 mmol, 2 equiv.) and Intermediate C (1 g, 2.144 mmol, 1 equiv.) in pyridine (20 mL) was stirred at 50° C. for 2 h. The resulting mixture was concentrated under vacuum. The crude material was purified by C18 reverse phase chromatography, 5% to 45% MeCN/H₂O to afford (1R,3S)-3-(3-(((benzyloxy)carbonyl)amino)-1H-pyrazol-5-yl)cyclopentyl 3-methyl-2,3-diazabicyclo[2.2.1]heptane-2-carboxylate (280 mg, 30% yield) as a light yellow solid.

### Step 4: Synthesis of (1R,3S)-3-(3-amino-1H-pyrazol-5-yl)cyclopentyl 3-methyl-2,3-diazabicyclo[2.2.1]heptane-2-carboxylate

To a stirred solution of (1R,3S)-3-(3-(((benzyloxy)carbonyl)amino)-1H-pyrazol-5-yl)cyclopentyl 3-methyl-2,3-diazabicyclo[2.2.1]heptane-2-carboxylate (280 mg, 0.637 mmol, 1 equiv.) in MeOH (10 mL) was added 10% Pd/C (350 mg). The mixture was maintained under hydrogen atmosphere at room temperature for 3 h. The resulting mixture was filtered, the filter cake was washed with DCM (2×10 mL). The filtrate was concentrated under reduced pressure. This resulted in (1R,3S)-3-(3-amino-1H-pyrazol-5-yl)cyclopentyl 3-methyl-2,3-diazabicyclo[2.2.1]heptane-2-carboxylate (170 mg, 87% yield) as a light yellow oil. The crude material was used without any further purification.

### Step 5: Synthesis of (1R,3S)-3-(3-(2-(2-formyl-3-hydroxy-5-methoxyphenoxy)acetamido)-1H-pyrazol-5-yl)cyclopentyl 3-methyl-2,3-diazabicyclo[2.2.1]heptane-2-carboxylate

To a stirred solution of Intermediate A (130.36 mg, 0.576 mmol, 1.1 equiv.) and (1R,3S)-3-(3-amino-1H-pyrazol-5-yl)cyclopentyl 3-methyl-2,3-diazabicyclo[2.2.1]heptane-2-carboxylate (160 mg, 0.524 mmol, 1 equiv.) in pyridine (4 mL) was added EDCI (122.01 mg, 0.786 mmol, 1.5 equiv.) at room temperature. The resulting mixture was stirred at room temperature for 1 h. The reaction was quenched with water and the mixture was extracted with EtOAc (2×15 mL). The combined organic layers were washed with brine (3×10 mL), dried over anhydrous Na₂SO₄, filtered, and concentrated under reduced pressure. The crude material was purified by C18 reverse phase chromatography, 5% to 46% MeCN/H₂O to afford (1R,3S)-3-(3-(2-(2-formyl-3-hydroxy-5-methoxyphenoxy)acetamido)-1H-pyrazol-5-yl)cyclopentyl 3-methyl-2,3-diazabicyclo[2.2.1]heptane-2-carboxylate (Compound 272, 43.1 mg, 13% yield) as an off-white solid. MS (ESI) calcd. for C₂₅H₃₁N₅O₇ 513.22 m/z, found [M+H]⁺ 514.25 m/z. 1H NMR (300 MHz, DMSO-d6) δ 12.31 (s, 1H), 10.57 (s, 1H), 10.14 (s, 1H), 6.33 (d, J=1.5 Hz, 1H), 6.13 (d, J=2.1 Hz, 1H), 6.06 (t, J=1.8 Hz, 1H), 5.15-5.06 (m, 1H), 4.80 (s, 2H), 4.34-4.21 (m, 1H), 3.82-3.75 (m, 4H), 3.19-3.08 (m, 1H), 2.83-2.59 (m, 3H), 2.45-2.35 (m, 1H), 2.23-2.11 (m, 1H), 2.10-1.99 (m, 1H), 1.96-1.63 (m, 6H), 1.61-1.42 (m, 3H).

### Example 28: Preparation of Compound 273

### Synthesis of (1R,3S)-3-(3-(2-(2-formyl-3-hydroxy-5-methoxyphenoxy)acetamido)-1H-pyrazol-5-yl)cyclopentyl 2,3-diazabicyclo[2.2.1]heptane-2-carboxylate

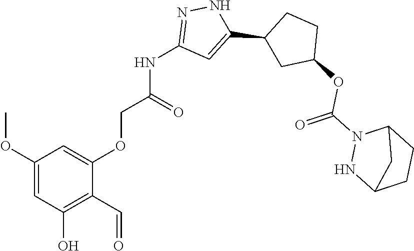 [SMILES: COc1cc(O)c(C=O)c(OCC(=O)Nc2cc([C@H]3CC[C@@H](OC(=O)N4NC5CCC4C5)C3)[nH]n2)c1]

### Synthetic Route

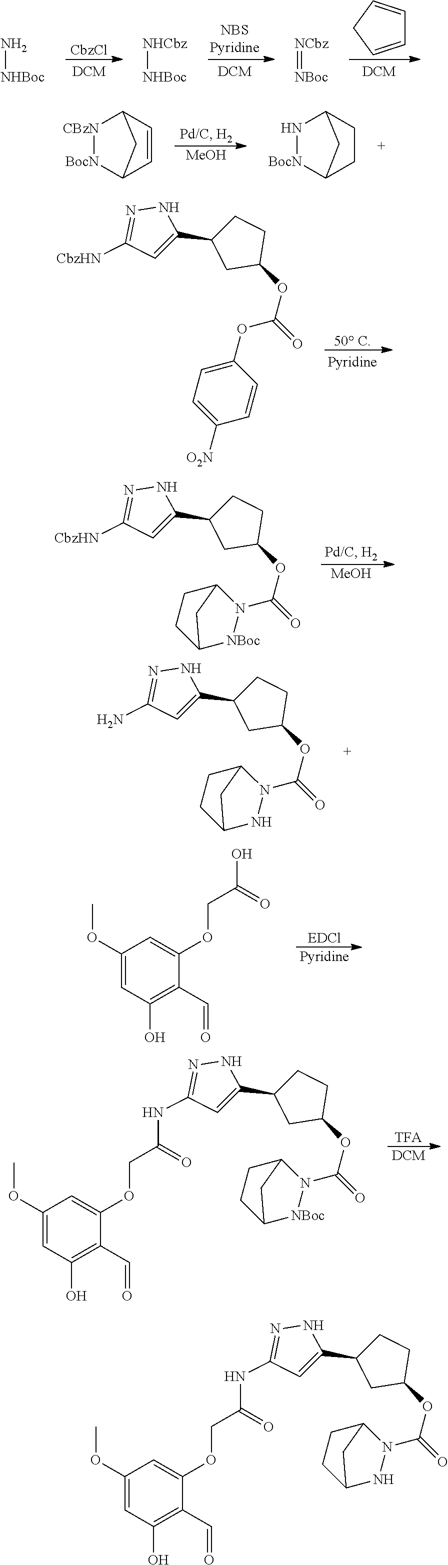

### Step 1: Synthesis of 1-benzyl 2-(tert-butyl) hydrazine-1,2-dicarboxylate

To a stirred solution of tert-butoxycarbohydrazide (20 g, 151.328 mmol, 1 equiv.) in DCM (250 mL) was added CbzCl (38.72 g, 226.992 mmol, 1.5 equiv.) dropwise at 0° C. and left to warm to room temperature and stir overnight. The reaction was quenched with water and extracted with DCM (2×300 mL), the combined organic extracts were washed with brine (2×300 mL), dried over anhydrous Na₂SO₄, filtered and concentrated under vacuum. The residue was purified by silica gel column chromatography, eluted with PE/EtOAc (3:1) to afford 1-benzyl 2-(tert-butyl) hydrazine-1,2-dicarboxylate (40 g, 99% yield) as a white solid.

### Step 2: Synthesis of 1-benzyl 2-(tert-butyl) (E)-diazene-1,2-dicarboxylate

To a stirred solution of 1-benzyl 2-(tert-butyl) hydrazine-1,2-dicarboxylate (12 g, 45.062 mmol, 1 equiv.) in DCM (50 mL) was added pyridine (3.64 g, 45.963 mmol, 1.02 equiv.) dropwise at 0° C. To the above mixture was added NBS (8.02 g, 45.062 mmol, 1 equiv.) in portions at 0° C. The resulting mixture was stirred at room temperature for 4 h. The reaction was quenched with water and then the mixture was extracted with DCM (2×300 mL). The combined organic extracts were washed with brine (2×300 mL), dried over anhydrous Na₂SO₄, and concentrated under vacuum to afford 1-benzyl 2-(tert-butyl) (E)-diazene-1,2-dicarboxylate (11 g, 92% yield) as a yellow solid. The crude material was used without any further purification.

### Step 3: Synthesis of 2-benzyl 3-(tert-butyl) 2,3-diazabicyclo[2.2.1]hept-5-ene-2,3-dicarboxylate

To a stirred solution of 1-benzyl 2-(tert-butyl) (E)-diazene-1,2-dicarboxylate (11 g, 41.622 mmol, 1 equiv.) in DCM (110 mL) was added freshly cracked cyclopentadiene (5.50 g, 83.244 mmol, 2 equiv.) dropwise at 0° C. under N₂ atmosphere. The resulting mixture was stirred at room temperature overnight under N₂ atmosphere. The reaction was quenched with water and then the mixture was extracted with DCM (2×300 mL). The combined organic extracts were washed with brine (2×300 mL), dried over anhydrous Na₂SO₄, filtered, and concentrated under vacuum to afford 2-benzyl 3-(tert-butyl) 2,3-diazabicyclo[2.2.1]hept-5-ene-2,3-dicarboxylate (11 g, 80% yield) as a brick red oil. The crude material was used without any further purification.

### Step 4: Synthesis of tert-butyl 2,3-diazabicyclo[2.2.1]heptane-2-carboxylate

To a stirred solution of 2-benzyl 3-(tert-butyl) 2,3-diazabicyclo[2.2.1]hept-5-ene-2,3-dicarboxylate (11 g, 33.295 mmol, 1 equiv.) in MeOH (100 mL) was added 10% Pd/C (8 g). The mixture was maintained under hydrogen atmosphere for 3 h under hydrogen atmosphere. The resulting mixture was filtered, the filter cake was washed with DCM (2×300 mL) and the filtrate was concentrated under reduced pressure. This resulted in tert-butyl 2,3-diazabicyclo[2.2.1]heptane-2-carboxylate (8 g, 90% yield) as a light yellow oil. The crude material was used without any further purification.

### Step 5: Synthesis of 2-((1R,3S)-3-(3-(((benzyloxy)carbonyl)amino)-1H-pyrazol-5-yl)cyclopentyl) 3-(tert-butyl) 2,3-diazabicyclo[2.2.1]heptane-2,3-dicarboxylate

A solution of Intermediate C (900 mg, 1.929 mmol, 1 equiv.) and tert-butyl 2,3-diazabicyclo[2.2.1]heptane-2-carboxylate (459.06 mg, 2.315 mmol, 1.2 equiv.) in pyridine (10 mL) was stirred at 50° C. for 2 h. The resulting mixture was cooled to room temperature and concentrated under vacuum. The crude material was purified by C18 reverse phase chromatography, 5 to 61% MeCN/H₂O to afford 2-((1R,3S)-3-(3-(((benzyloxy)carbonyl)amino)-1H-pyrazol-5-yl)cyclopentyl) 3-(tert-butyl) 2,3-diazabicyclo[2.2.1]heptane-2,3-dicarboxylate (600 mg, 59% yield) as a light yellow solid.

### Step 6: Synthesis of 2-((1R,3S)-3-(3-amino-1H-pyrazol-5-yl)cyclopentyl) 3-(tert-butyl) 2,3-diazabicyclo[2.2.1]heptane-2,3-dicarboxylate

To a stirred solution of 2-((1R,3S)-3-(3-(((benzyloxy)carbonyl)amino)-1H-pyrazol-5-yl)cyclopentyl) 3-(tert-butyl) 2,3-diazabicyclo[2.2.1]heptane-2,3-dicarboxylate (550 mg, 1.046 mmol, 1 equiv.) in MeOH (5 mL) was added 10% Pd/C (500 mg). The mixture was maintain under hydrogen atmosphere for 3 h at room temperature. The resulting mixture was filtered, the filter cake was washed with DCM (2×10 mL) and the filtrate was concentrated under reduced pressure resulting in 2-((1R,3S)-3-(3-amino-1H-pyrazol-5-yl)cyclopentyl) 3-(tert-butyl) 2,3-diazabicyclo[2.2.1]heptane-2,3-dicarboxylate (390 mg, 95% yield) as a yellow oil. The crude material was used without any further purification.

### Step 7: Synthesis of 2-(tert-butyl) 3-((1R,3S)-3-(3-(2-(2-formyl-3-hydroxy-5-methoxyphenoxy)acetamido)-1H-pyrazol-5-yl)cyclopentyl) 2,3-diazabicyclo[2.2.1]heptane-2,3-dicarboxylate

To a stirred solution of 2-((1R,3S)-3-(3-amino-1H-pyrazol-5-yl)cyclopentyl) 3-(tert-butyl) 2,3-diazabicyclo[2.2.1]heptane-2,3-dicarboxylate (200 mg, 0.511 mmol, 1 equiv.) and Intermediate A (127.11 mg, 0.562 mmol, 1.1 equiv.) in pyridine (5 mL) was added EDCI (118.97 mg, 0.766 mmol, 1.5 equiv.) at room temperature. The resulting mixture was stirred at room temperature for 1 h. The reaction was quenched with water and extracted with EtOAc (2×20 mL). The combined organic extracts were washed with brine (2×20 mL), dried over anhydrous Na₂SO₄, filtered and concentrated under reduced atmosphere to afford 2-(tert-butyl) 3-((1R,3S)-3-(3-(2-(2-formyl-3-hydroxy-5-methoxyphenoxy)acetamido)-1H-pyrazol-5-yl)cyclopentyl) 2,3-diazabicyclo[2.2.1]heptane-2,3-dicarboxylate (200 mg, 65% yield) as a brown oil. The crude material was used without any further purification.

### Step 8: Synthesis of (1R,3S)-3-(3-(2-(2-formyl-3-hydroxy-5-methoxyphenoxy)acetamido)-1H-pyrazol-5-yl)cyclopentyl 2,3-diazabicyclo[2.2.1]heptane-2-carboxylate

A solution of 2-(tert-butyl) 3-((1R,3S)-3-(3-(2-(2-formyl-3-hydroxy-5-methoxyphenoxy)acetamido)-1H-pyrazol-5-yl)cyclopentyl) 2,3-diazabicyclo[2.2.1]heptane-2,3-dicarboxylate (200 mg, 0.334 mmol, 1 equiv.) in DCM (3 mL) and TFA (1 mL) was stirred at room temperature for 1 h. The resulting mixture was concentrated under reduced atmosphere and the resulting crude material was purified by C18 reverse phase chromatography, 5 to 30% MeCN/H₂O to afford (1R,3S)-3-(3-(2-(2-formyl-3-hydroxy-5-methoxyphenoxy)acetamido)-1H-pyrazol-5-yl)cyclopentyl 2,3-diazabicyclo[2.2.1]heptane-2-carboxylate (Compound 273, 41.2 mg, 20% yield) as a light yellow solid. MS (ESI) calcd. for C₂₄H₂₉N₅O₇ 499.21 m/z, found [M+H]⁺ 500.20 m/z. 1H NMR (300 MHz, DMSO-d6) δ 12.31 (s, 1H), 10.59 (s, 1H), 10.15 (s, 1H), 6.35 (s, 1H), 6.16-6.15 (m, 1H), 6.09-6.08 (m, 1H), 5.20-5.14 (m, 1H), 4.82 (s, 2H), 4.50-4.48 (m, 1H), 4.30 (s, 1H), 3.81 (s, 3H), 3.19-3.14 (m, 1H), 2.47-2.42 (m, 1H), 2.08-1.65 (m, 11H).

### Example 29: Preparation of Compound 274

### Synthesis of (1R,3S)-3-(3-(2-(2-formyl-3-hydroxy-5-methoxyphenoxy)acetamido)-1H-pyrazol-5-yl)cyclopentyl 2-ethyl-2-methylhydrazine-1-carboxylate

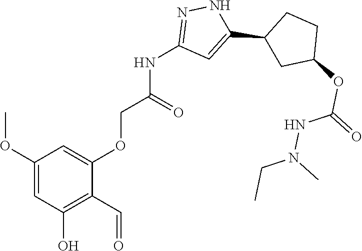 [SMILES: CCN(C)NC(=O)O[C@@H]1CC[C@H](c2cc(NC(=O)COc3cc(OC)cc(O)c3C=O)n[nH]2)C1]

### Synthetic Route

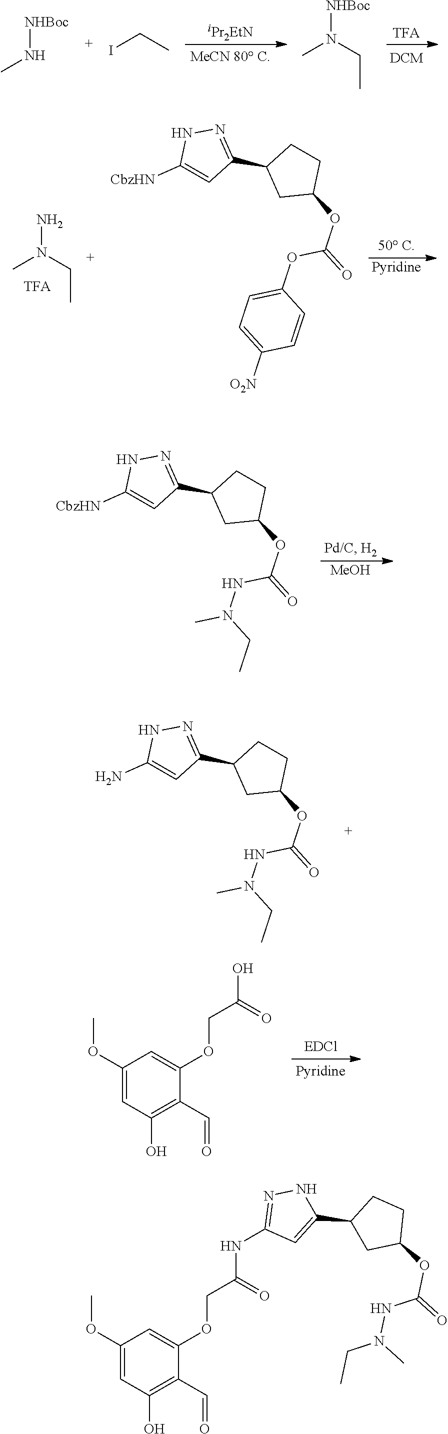

### Step 1: Synthesis of tert-butyl 2-ethyl-2-methylhydrazine-1-carboxylate

To a stirred solution of tert-butyl 2-methylhydrazine-1-carboxylate (2 g, 13.681 mmol, 1.0 equiv.) and iodoethane (3.2 g, 20.517 mmol, 1.5 equiv.) in MeCN (30 mL) was added DIEA (4.7 mL, 27.080 mmol, 1.98 equiv.) dropwise at room temperature under air atmosphere. The resulting mixture was heated to 90° C. and stirred for 2 h. After cooling down to room temperature, the solvent was removed in vacuo. The residue was resuspended with DCM (300 mL), washed with water (3×100 mL), brine (100 mL), dried over anhydrous Na₂SO₄, filtered, and concentrated under reduced pressure to give tert-butyl 2-ethyl-2-methylhydrazine-1-carboxylate (1.87 g, 78.6% yield) as a colorless oil. The crude material was used without any further purification.

### Step 2: Synthesis of 1-ethyl-1-methylhydrazine 2,2,2-trifluoroacetate

To a stirred solution of tert-butyl 2-ethyl-2-methylhydrazine-1-carboxylate (1.8 g, 10.345 mmol, 1 equiv.) in DCM (9 mL) was added TFA (3 mL) at room temperature. The resulting mixture was stirred at room temperature for 2 h. The resulting mixture was concentrated under reduced pressure to give 1-ethyl-1-methylhydrazine 2,2,2-trifluoroacetate (1.6 g, 90.4% yield) as a yellow oil. The crude material was used without any further purification.

### Step 3: Synthesis of benzyl N-{5-[(1S,3R)-3-[(N′-ethyl-N′-methylhydrazinecarbonyl)oxy]cyclopentyl]-2H-pyrazol-3-yl}carbamate

To a stirred solution of intermediate C (850 mg, 1.822 mmol, 1 equiv.) in pyridine (10 mL) was added 1-ethyl-1-methylhydrazine 2,2,2-trifluoroacetate (1.6 g, 9.356 mmol, 5.135 equiv.) at room temperature. The resulting mixture was heated to 50° C. and stirred for 2 h. After cooling down to room temperature, the solvent was removed in vacuo. The residue was resuspended with EtOAc (300 mL), washed with water (3×100 mL), brine (100 mL), dried over anhydrous Na₂SO₄, filtered, and concentrated under reduced pressure. The residue was purified by reversed-phase flash chromatography with MeCN in Water (10 mmol/L NH₄HCO₃) to afford benzyl N-{5-[(1S,3R)-3-[(N′-ethyl-N′-methylhydrazinecarbonyl)oxy]cyclopentyl]-2H-pyrazol-3-yl}carbamate (350 mg, 47.84% yield) as a yellow solid.

### Step 4: Synthesis of 1-{[(1R,3S)-3-(5-amino-1H-pyrazol-3-yl)cyclopentyl]oxy}-N′-ethyl-N′-methylformohydrazide

To a stirred solution of benzyl N-{5-[(1S,3R)-3-[(N′-ethyl-N′-methylhydrazinecarbonyl)oxy]cyclopentyl]-2H-pyrazol-3-yl}carbamate (350 mg, 0.872 mmol, 1 equiv.) in methanol (5 mL) was added 10% Pd/C (350 mg). The resulting mixture was stirred at room temperature for 2 h under hydrogen atmosphere. The reaction mixture was filtered, the filter cake was washed with DCM (5×5 mL), and the filtrate was concentrated under reduced pressure to afford 1-{[(1R,3S)-3-(5-amino-1H-pyrazol-3-yl)cyclopentyl]oxy}-N′-ethyl-N′-methylformohydrazide (230 mg, 98.79% yield) as a yellow oil. The crude material was used without any further purification.

### Step 5: Synthesis of N-{5-[(1S,3R)-3-[(N′-ethyl-N′-methylhydrazinecarbonyl)oxy]cyclopentyl]-2H-pyrazol-3-yl}-2-(2-formyl-3-hydroxy-5-methoxyphenoxy)acetamide

To a stirred solution of 1-{[(1R,3S)-3-(5-amino-1H-pyrazol-3-yl)cyclopentyl]oxy}-N′-ethyl-N′-methylformohydrazide (230 mg, 0.861 mmol, 1 equiv.) and intermediate A (195 mg, 0.948 mmol, 1.10 equiv.) in pyridine (5 mL) was added EDCI (248 mg, 1.292 mmol, 1.50 equiv). The resulting mixture was stirred at room temperature for 2 h. The solvent was removed in vacuo. The residue was diluted with EtOAc (50 mL). The organic layer was washed with water (5×10 mL) and brine (10 mL) dried over anhydrous Na₂SO₄. After filtration, the filtrate was concentrated under reduced pressure. The residue was purified by reversed-phase flash chromatography with MeCN in Water (0.1% TFA) to afford (1R,3S)-3-(3-(2-(2-formyl-3-hydroxy-5-methoxyphenoxy)acetamido)-1H-pyrazol-5-yl)cyclopentyl 2-ethyl-2-methylhydrazine-1-carboxylate (Compound 274, 51.4 mg, 10.43% yield) as a yellow solid. 1H NMR (300 MHz, DMSO-d6) δ 12.32 (s, 1H), 10.56 (s, 1H), 10.14 (s, 1H), 8.91 (br s, 1H), 6.31 (s, 1H), 6.14 (d, J=2.1 Hz, 1H), 6.06 (d, J=2.1 Hz, 1H), 5.11-5.03 (m, 1H), 4.80 (s, 2H), 3.80 (s, 3H), 3.21-3.07 (m, 1H), 2.77-2.72 (m, 2H), 2.57 (s, 3H), 2.48-2.35 (m, 1H), 2.07-2.02 (m, 1H), 1.95-1.82 (m, 1H), 1.82-1.55 (m, 3H), 0.99 (t, J=6.0 Hz, 3H). MS(ESI) calcd for C₂₂H₂₉N₅O₇ 475.21 m/z, found [M+H]⁺ 476.15 m/z.

### Example 30: Preparation of Compound 275

### Synthesis of (1R,3S)-3-(3-(2-(2-formyl-3-hydroxy-5-methoxyphenoxy)acetamido)-1H-pyrazol-5-yl)cyclopentyl 1-ethyl-2-methylhydrazine-1-carboxylate

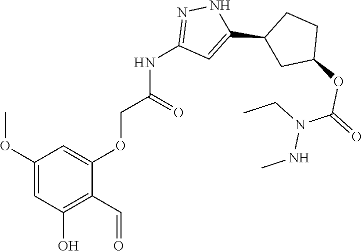 [SMILES: CCN(NC)C(=O)O[C@@H]1CC[C@H](c2cc(NC(=O)COc3cc(OC)cc(O)c3C=O)n[nH]2)C1]

### Synthetic Route

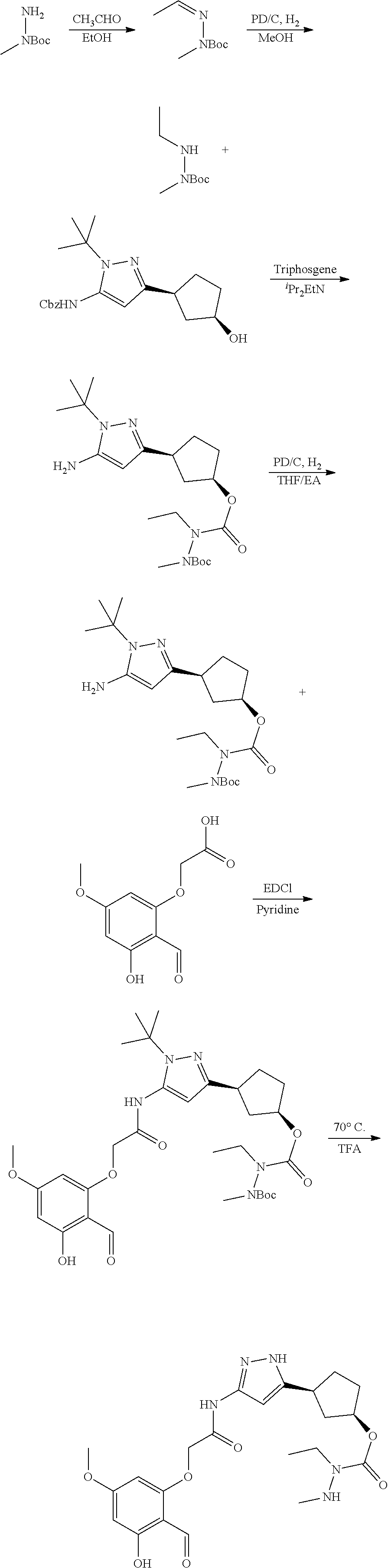

### Step 1: tert-butyl 2-ethylidene-1-methylhydrazine-1-carboxylate

To a stirred solution of N-methyl tert-butoxycarbohydrazide (10 g, 68.4 mmol, 1 equiv.) in ethyl alcohol (100 mL) was added acetaldehyde 40% solution in H₂O (11 mL, 102.6 mmol, 1.5 equiv.) in portions at room temperature and left to stir 5 h. The resulting mixture was concentrated under vacuum and purified by silica gel column chromatography, eluted with PE/EtOAc (30%) to afford tert-butyl 2-ethylidene-1-methylhydrazine-1-carboxylatebutoxycarbohydrazide (8.0 g, 67.91% yield) as a white oil.

### Step 2: Synthesis of tert-butyl 2-ethyl-1-methylhydrazine-1-carboxylate

To a stirred solution of N′-[(1E)-ethylidene]-N-methyltert-butoxycarbohydrazide (5.0 g, 22.6 mmol, 1 equiv.) in MeOH (50 mL) was added Pd/C (2.0 g) in portions at room temperature. The resulting mixture was stirred at room temperature for 3 h under H₂ atmosphere. The resulting mixture was filtered, the filter cake was washed with MeOH (3×10 mL) and the filtrate was concentrated under reduced atmosphere. The crude material was used without any further purification.

### Step 3: Synthesis of 1-((1R,3S)-3-(5-(((benzyloxy)carbonyl)amino)-1-(tert-butyl)-1H-pyrazol-3-yl)cyclopentyl) 2-(tert-butyl) 1-ethyl-2-methylhydrazine-1,2-dicarboxylate

To a stirred solution of benzyl N-{2-tert-butyl-5-[(1S,3R)-3-hydroxycyclopentyl]pyrazol-3-yl}carbamate (1.54 g, 4.304 mmol, 0.5 equiv.) and DIEA (3.34 g, 25.842 mmol, 3.00 equiv.) in DCM (100 mL) was added triphosgene (1.28 g, 4.304 mmol, 0.5 equiv.) in portions at room temperature. The resulting mixture was stirred at room temperature for 2 h then concentrated under reduced atmosphere. N′-ethyl-N-methyltert-butoxycarbohydrazide (1.5 g, 8.609 mmol, 1 equiv.) and DIEA (3.34 g, 25.842 mmol, 3.00 equiv.) in DCM (50 mL) was added to the above material in portions at room temperature. The resulting mixture was stirred at room temperature for 8 h. The resulting mixture was diluted with water and extracted with DCM (3×100 mL). The combined organic layers were washed with brine (2×100 mL), dried over anhydrous Na₂SO₄, filtered, and concentrated under reduced pressure. The crude material was diluted with DMF and water, filtered through a syringe filter and purified by C18 reverse phase chromatography, 10 to 70% MeCN/H₂O with 0.05% TFA modifier to afford benzyl N-{5-[(1S,3R)-3-{[N′-(tert-butoxycarbonyl)-N-ethyl-N′-methylhydrazinecarbonyl]oxy}cyclopentyl]-2-tert-butylpyrazol-3-yl}carbamate (0.6 g, 12.50% yield) as a white solid.

### Step 4: Synthesis of 1-((1R,3S)-3-(5-amino-1-(tert-butyl)-1H-pyrazol-3-yl)cyclopentyl) 2-(tert-butyl) 1-ethyl-2-methylhydrazine-1,2-dicarboxylate

To a stirred solution of benzyl N-{5-[(1S,3R)-3-{[N′-(tert-butoxycarbonyl)-N-ethyl-N′-methylhydrazinecarbonyl]oxy}cyclopentyl]-2-tert-butylpyrazol-3-yl}carbamate (0.55 g, 0.986 mmol, 1 equiv.) in EtOAc (5 mL) was added 10% Pd/C (274.97 mg) in portions at room temperature. The resulting mixture was stirred at room temperature for 2 h under H₂ atmosphere. The resulting mixture was filtered, the filter cake was washed with MeOH (3×10 mL) and the filtrate was concentrated under reduced pressure to afford 1-{[(1R,3S)-3-(5-amino-1-tert-butylpyrazol-3-yl)cyclopentyl]oxy}-N′-(tert-butoxycarbonyl)-N-ethyl-N′-methylformohydrazide (0.35 g, 80.0% yield) as a yellow oil. The crude material was used without any further purification.

### Step 5: Synthesis of 1-(tert-butyl) 2-((1R,3S)-3-(1-(tert-butyl)-5-(2-(2-formyl-3-hydroxy-5-methoxyphenoxy)acetamido)-1H-pyrazol-3-yl)cyclopentyl) 2-ethyl-1-methylhydrazine-1,2-dicarboxylate

To a stirred solution of 1-{[(1R,3S)-3-(5-amino-1-tert-butylpyrazol-3-yl)cyclopentyl]oxy}-N′-(tert-butoxycarbonyl)-N-ethyl-N′-methylformohydrazide (200 mg, 0.472 mmol, 1 equiv.) and 2-formyl-3-hydroxy-5-methoxyphenoxyacetic acid (128.16 mg, 0.566 mmol, 1.2 equiv.) in pyridine (5 mL) was added EDCI (146.61 mg, 0.944 mmol, 2.0 equiv.) in portions at room temperature. The resulting mixture was stirred at room temperature for 3 h. The resulting mixture was diluted with water and extracted with EtOAc (3×20 mL). The combined organic layers were washed with brine (2×20 mL), dried over anhydrous Na₂SO₄, filtered, and concentrated under reduced pressure. The recovered material was diluted with DMF and water, filtered through a syringe filter and purified by C18 reverse phase chromatography, 10 to 70% MeCN/H₂O with 0.05% TFA modifier to afford N-{5-[(1S,3R)-3-{[N′-(tert-butoxycarbonyl)-N-ethyl-N′-methylhydrazinecarbonyl]oxy}cyclopentyl]-2-tert-butylpyrazol-3-yl}-2-(2-formyl-3-hydroxy-5-methoxyphenoxy)acetamide (90 mg, 30.17% yield) as a brown solid.

### Step 6: Synthesis of (1R,3S)-3-(3-(2-(2-formyl-3-hydroxy-5-methoxyphenoxy)acetamido)-1H-pyrazol-5-yl)cyclopentyl 1-ethyl-2-methylhydrazine-1-carboxylate

N-{5-[(1S,3R)-3-{[N′-(tert-butoxycarbonyl)-N-ethyl-N′-methylhydrazinecarbonyl]oxy}cyclopentyl]-2-tert-butylpyrazol-3-yl}-2-(2-formyl-3-hydroxy-5-methoxyphenoxy)acetamide (90 mg, 0.142 mmol, 1 equiv.) in trifluoroacetic acid (2 mL) was stirred at 70° C. for 3 h. The resulting mixture was cooled to room temperature and concentrated under vacuum. The crude product was purified by Prep-HPLC, 5 to 60% MeCN/H₂O with 0.05% TFA to afford (1R,3S)-3-(3-(2-(2-formyl-3-hydroxy-5-methoxyphenoxy)acetamido)-1H-pyrazol-5-yl)cyclopentyl 1-ethyl-2-methylhydrazine-1-carboxylate (Compound 275, 26.4 mg, 31.43% yield) as an off-white solid. 1H NMR (300 MHz, DMSO-d6) δ 12.32 (s, 1H), 10.56 (s, 1H), 10.15 (s, 1H), 6.33 (s, 1H), 6.15-6.14 (s, 1H), 6.07-6.06 (m, 1H), 5.10-5.08 (m, 1H), 4.81 (s, 2H), 3.81 (s, 3H), 3.33-3.25 (m, 2H), 3.16-3.10 (m, 1H), 2.52 (s, 3H), 2.50-2.40 (m, 1H), 2.08-2.00 (m, 1H), 1.99-1.78 (m, 4H), 1.15-0.90 (m, 3H). MS (ESI) calcd. for C₂₂H₂₉N₅O₇, 475.21 m/z, found [M+H]⁺ 476.25 m/z.

### Example 31: Preparation of Compound 276

### Synthesis of (1R,3S)-3-(3-(2-(2-formyl-3-hydroxy-5-methoxyphenoxy)acetamido)-1H-pyrazol-5-yl)cyclopentyl 2-ethyl-1-methylhydrazine-1-carboxylate

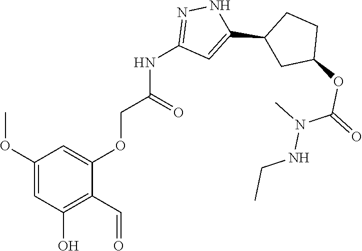 [SMILES: CCNN(C)C(=O)O[C@@H]1CC[C@H](c2cc(NC(=O)COc3cc(OC)cc(O)c3C=O)n[nH]2)C1]

### Synthetic Route

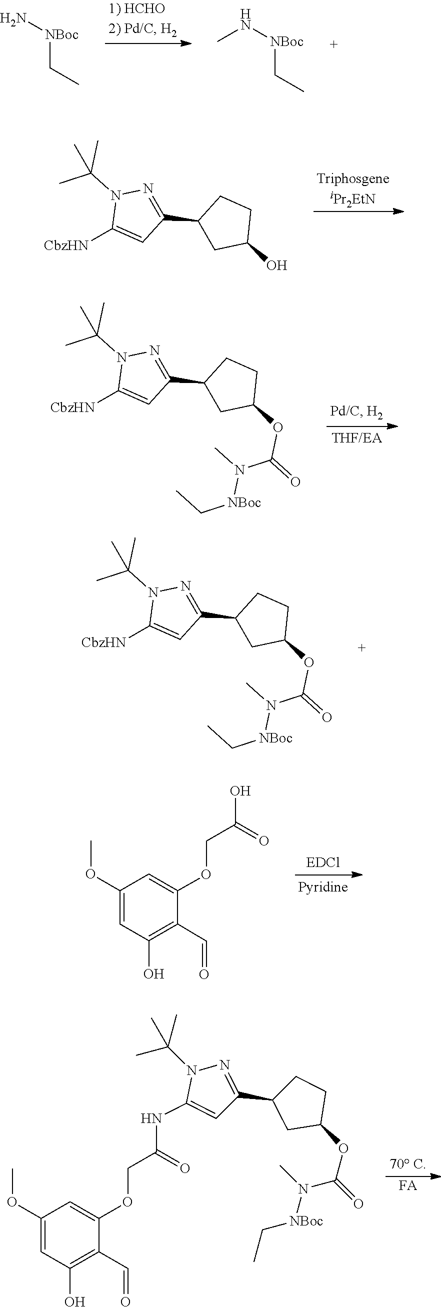

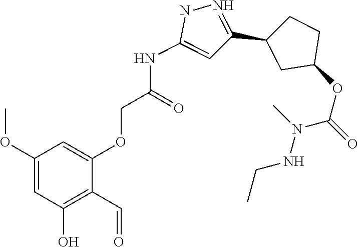 [SMILES: CCNN(C)C(=O)O[C@@H]1CC[C@H](c2cc(NC(=O)COc3cc(OC)cc(O)c3C=O)[nH]n2)C1]

### Step 1: Synthesis of tert-butyl 1-ethyl-2-methylhydrazine-1-carboxylate

To a stirred solution of N-ethyltert-butoxycarbohydrazide (3 g, 18.725 mmol, 1 equiv.) in methanol (30 mL) was added formaldehyde 40% solution in H₂O (2.58 mL, 37.450 mmol, 2 equiv.) at room temperature. The resulting mixture was stirred for 2 h at room temperature to which 10% Pd/C (1.2 g) was added. The resulting mixture was stirred for 5 h at room temperature under H₂ atmosphere. The resulting mixture was filtered, the filter cake was washed with DCM (3×30 mL) and the filtrate was concentrated under reduced pressure. This resulted in tert-butyl 1-ethyl-2-methylhydrazine-1-carboxylate (1.2 g, 37% yield) as a yellow oil. The crude product was used in the next step directly without further purification.

### Step 2: Synthesis of 1-((1R,3S)-3-(5-(((benzyloxy)carbonyl)amino)-1-(tert-butyl)-1H-pyrazol-3-yl)cyclopentyl) 2-(tert-butyl) 2-ethyl-1-methylhydrazine-1,2-dicarboxylate

A solution of benzyl N-{2-tert-butyl-5-[(1S,3R)-3-hydroxycyclopentyl]pyrazol-3-yl}carbamate (800 mg, 0.280 mmol, 1 equiv.) in DCM (10 mL) was treated with triphosgene (748 mg, 2.378 mmol, 1 equiv.) for 10 min at 0° C. under nitrogen atmosphere followed by the addition of DIEA (2.6 mL, 1.680 mmol, 6 equiv.) dropwise at 0° C. and was subsequently stirred for 1 h. The resulting mixture was concentrated under reduced pressure. The resulting crude material was resuspended in DCM (10 mL) and treated with tert-butyl 1-ethyl-2-methylhydrazine-1-carboxylate (1.2 g, 4.756 mmol, 3 equiv.) and DIEA (2.6 mL, 1.680 mmol, 6 equiv.) at 25° C. under nitrogen atmosphere. The resulting mixture was stirred for 1 h at room temperature. Then the resulting mixture was diluted with EtOAc (50 mL) and washed with H₂O (3×50 mL), dried over anhydrous Na₂SO₄, filtered, and was concentrated under reduced pressure. The crude material was purified by C18 reverse phase chromatography, 5 to 80% MeCN/H₂O to afford 1-((1R,3S)-3-(5-(((benzyloxy)carbonyl)amino)-1-(tert-butyl)-1H-pyrazol-3-yl)cyclopentyl) 2-(tert-butyl) 2-ethyl-1-methylhydrazine-1,2-dicarboxylate (390 mg, 31.20% yield) as a yellow oil.

### Step 3: Synthesis of 1-((1R,3S)-3-(5-amino-1-(tert-butyl)-1H-pyrazol-3-yl)cyclopentyl) 2-(tert-butyl) 2-ethyl-1-methylhydrazine-1,2-dicarboxylate

To a stirred solution of 1-((1R,3S)-3-(5-(((benzyloxy)carbonyl)amino)-1-(tert-butyl)-1H-pyrazol-3-yl)cyclopentyl) 2-(tert-butyl) 2-ethyl-1-methylhydrazine-1,2-dicarboxylate (390 mg, 0.663 mmol, 1 equiv.) in tetrahydrofuran (5 mL) was added 10% Pd/C (500 mg) at 25° C. The resulting mixture was stirred at 25° C. for 3 h under H₂ atmosphere. The resulting mixture was filtered, the filter cake was washed with EtOAc (3×30 mL), and the filtrate was concentrated under reduced pressure to afford 1-((1R,3S)-3-(5-amino-1-(tert-butyl)-1H-pyrazol-3-yl)cyclopentyl) 2-(tert-butyl) 2-ethyl-1-methylhydrazine-1,2-dicarboxylate (185 mg, 65.83% yield) as a yellow oil. The crude material was used without any further purification.

### Step 4: Synthesis of 1-(tert-butyl) 2-((1R,3S)-3-(1-(tert-butyl)-5-(2-(2-formyl-3-hydroxy-5-methoxyphenoxy)acetamido)-1H-pyrazol-3-yl)cyclopentyl) 1-ethyl-2-methylhydrazine-1,2-dicarboxylate

To a stirred solution of 1-((1R,3S)-3-(5-amino-1-(tert-butyl)-1H-pyrazol-3-yl)cyclopentyl) 2-(tert-butyl) 2-ethyl-1-methylhydrazine-1,2-dicarboxylate (160 mg, 0.302 mmol, 1 equiv.) and 3-(benzyloxy)-2-formyl-5-methoxyphenoxyacetic acid (88 mg, 0.332 mmol, 1.2 equiv.) in pyridine (5 mL) were added EDCI (106 mg, 0.453 mmol, 1.7 equiv.) at 25° C. The resulting mixture was stirred for overnight at 25° C. The reaction was quenched with water and then the mixture was extracted with EtOAc (3×30 mL). The combined organic extracts were washed with brine (2×30 mL), dried over anhydrous Na₂SO₄, and concentrated under reduced atmosphere. The crude material was purified by C18 reverse phase chromatography, 5 to 70% MeCN/H₂O to afford 1-(tert-butyl) 2-((1R,3S)-3-(1-(tert-butyl)-5-(2-(2-formyl-3-hydroxy-5-methoxyphenoxy)acetamido)-1H-pyrazol-3-yl)cyclopentyl) 1-ethyl-2-methylhydrazine-1,2-dicarboxylate (80 mg, 30% yield) as a yellow oil.

### Step 5: Synthesis of N-{5-[(1S,3R)-3-[(N′-ethyl-N-methylhydrazinecarbonyl)oxy]cyclopentyl]-1H-pyrazol-3-yl}-2-(2-formyl-3-hydroxy-5-methoxyphenoxy)acetamide

A solution of N-{5-[(1S,3R)-3-{[N′-(tert-butoxycarbonyl)-N′-ethyl-N-methylhydrazinecarbonyl]oxy}cyclopentyl]-2-tert-butylpyrazol-3-yl}-2-(2-formyl-3-hydroxy-5-methoxyphenoxy)acetamide (70 mg, 0.111 mmol, 1 equiv.) in trifluoroacetic acid (3 mL, 0.026 mmol, 0.24 equiv.) was stirred for 6 h at 70° C. The resulting mixture was cooled to room temperature and concentrated under reduced pressure. The crude material was purified by C18 reverse phase chromatography, 5 to 60% MeCN/H₂O to afford (1R,3S)-3-(3-(2-(2-formyl-3-hydroxy-5-methoxyphenoxy)acetamido)-1H-pyrazol-5-yl)cyclopentyl 2-ethyl-1-methylhydrazine-1-carboxylate (Compound 276, 17.5 mg, 26.79% yield) as a yellow solid. MS (ESI) calcd. for C₂₂H₂₉N₅O₇ 475.21 m/z, found [M+H]⁺ 476.25 m/z. 1H NMR (400 MHz, DMSO-d6) δ 12.32 (s, 1H), 10.56 (s, 1H), 10.15 (s, 1H), 6.33 (s, 1H), 6.15-6.14 (m, 1H), 6.07-6.06 (m, 1H), 5.07-5.06 (m, 1H), 4.81 (s, 2H), 3.81 (s, 3H), 3.16-3.12 (m, 1H), 2.96 (s, 3H), 2.85 (s, 2H), 2.50-2.40 (m, 1H), 2.05-2.01 (m, 1H), 1.90-1.74 (m, 4H), 1.00-0.94 (m, 3H).

### Example 32: Preparation of Compound 277

### Synthesis of (1R,3S)-3-(3-(2-(2-formyl-3-hydroxy-5-methoxyphenoxy)acetamido)-1H-pyrazol-5-yl)cyclopentyl 2,2-diethyl-1-methylhydrazine-1-carboxylate

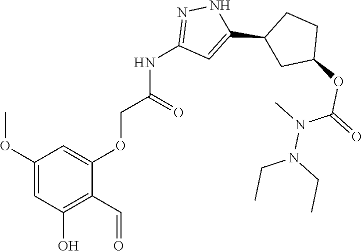 [SMILES: CCN(CC)N(C)C(=O)O[C@@H]1CC[C@H](c2cc(NC(=O)COc3cc(OC)cc(O)c3C=O)n[nH]2)C1]

### Synthetic Route

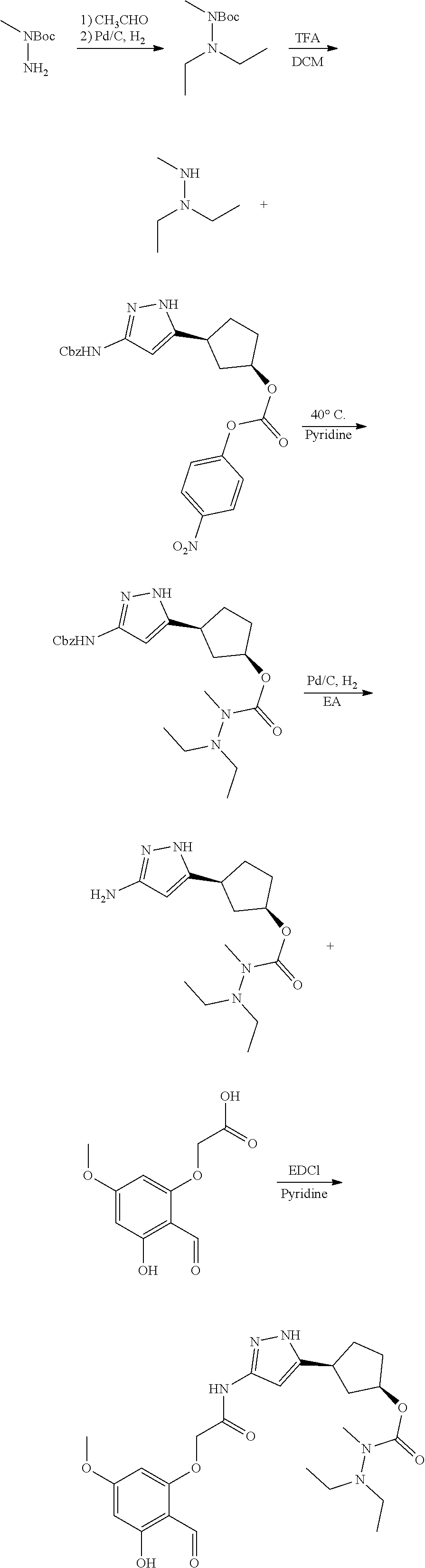

### Step 1: Synthesis of tert-butyl 2,2-diethyl-1-methylhydrazine-1-carboxylate

To a stirred solution of N-methyltert-butoxycarbohydrazide (10 g, 68.404 mmol, 1 equiv.) in EtOH (500 mL) was added acetaldehyde 40% solution in H₂O (11 mL, 102.6 mmol, 1.5 equiv.) at room temperature. The resulting mixture was stirred at room temperature for 5 h. To the reaction mixture was then 10% Pd/C (5.02 g) at room temperature. The resulting mixture was stirred at room temperature for 16 h under H₂ atmosphere. The resulting mixture was filtered, the filter cake was washed with MeOH (3×200 mL), and filtrate was concentrated under reduced pressure. The residue was purified by silica gel column chromatography, eluted with PE/EtOAc (10:1) to afford N′,N′-diethyl-N-methyltert-butoxycarbohydrazide (8 g, 57.81% yield) as a white oil.

### Step 2: Synthesis of 1,1-diethyl-2-methylhydrazine

To a stirred solution of N′,N′-diethyl-N-methyltert-butoxycarbohydrazide (1.5 g, 7.415 mmol, 1 equiv.) in DCM (10 mL) was added TFA (3 mL) at room temperature. The resulting mixture was stirred at room temperature for 2 h followed by concentration under reduced atmosphere. The crude material was used without any further purification.

### Step 3: Synthesis of (1R,3S)-3-(3-(((benzyloxy)carbonyl)amino)-1H-pyrazol-5-yl)cyclopentyl 2,2-diethyl-1-methylhydrazine-1-carboxylate

To a stirred solution of (1R,3S)-3-(5-{[(benzyloxy)carbonyl]amino}-2H-pyrazol-3-yl)cyclopentyl 4-nitrophenyl carbonate (1 g, 2.144 mmol, 1 equiv.) in pyridine (20 mL) was added 1,1-diethyl-2-methylhydrazine (2.19 g, 21.440 mmol, 10.0 equiv.) at room temperature. The resulting mixture was stirred at 40° C. for 5 h. The resulting mixture was diluted with water and extracted with EtOAc (3×20 mL). The combined organic layers were washed with 0.5 M NaOH (3×20 mL), dried over anhydrous Na₂SO₄, filtered, and concentrated under reduced pressure. The crude material was diluted with DMF and water, filtered through a syringe filter and purified by C18 reverse phase chromatography, 10 to 70% MeCN/H₂O with 0.05% TFA modifier to afford benzyl N-{5-[(1S,3R)-3-[(N′,N′-diethyl-N-methylhydrazinecarbonyl)oxy]cyclopentyl]-1H-pyrazol-3-yl}carbamate (0.35 g, 38.01% yield) as a white solid.

### Step 4: Synthesis of (1R,3S)-3-(3-amino-1H-pyrazol-5-yl)cyclopentyl 2,2-diethyl-1-methylhydrazine-1-carboxylate

To a stirred solution of benzyl N-{5-[(1S,3R)-3-[(N′,N′-diethyl-N-methylhydrazinecarbonyl)oxy]cyclopentyl]-1H-pyrazol-3-yl}carbamate (0.3 g, 0.698 mmol, 1 equiv.) in EtOAc (3 mL) was added 10% Pd/C (0.15 g) at room temperature. The resulting mixture was stirred at room temperature for 3 h under H₂ atmosphere. The resulting mixture was filtered, the filter cake was washed with MeOH (3×10 mL) and the filtrate was concentrated under reduced pressure to afford 1-{[(1R,3S)-3-(5-amino-2H-pyrazol-3-yl)cyclopentyl]oxy}-N′,N′-diethyl-N-methylformohydrazide (0.18 g, 87.25% yield) as a brown solid. The crude material was used without any further purification.

### Step 5: Synthesis of (1R,3S)-3-(3-(2-(2-formyl-3-hydroxy-5-methoxyphenoxy)acetamido)-1H-pyrazol-5-yl)cyclopentyl 2,2-diethyl-1-methylhydrazine-1-carboxylate

To a stirred solution of 1-{[(1R,3S)-3-(5-amino-2H-pyrazol-3-yl)cyclopentyl]oxy}-N′,N′-diethyl-N-methylformohydrazide (100 mg, 0.339 mmol, 1 equiv.) and 2-formyl-3-hydroxy-5-methoxyphenoxyacetic acid (76.57 mg, 0.339 mmol, 1.0 equiv.) in pyridine (3 mL) was added EDCI (78.83 mg, 0.509 mmol, 1.5 equiv.) at room temperature. The resulting mixture was stirred at room temperature for 2 h. The resulting mixture was diluted with water and extracted with EtOAc (3×20 mL). The combined organic layers were washed with brine (2×20 mL), dried over anhydrous Na₂SO₄, filtered, and concentrated under reduced pressure. The crude product was purified by Prep-HPLC, 5 to 60% MeCN/H₂O with 0.05% TFA to afford (1R,3S)-3-(3-(2-(2-formyl-3-hydroxy-5-methoxyphenoxy)acetamido)-1H-pyrazol-5-yl)cyclopentyl 2,2-diethyl-1-methylhydrazine-1-carboxylate (Compound 277, 37.0 mg, 17.70% yield) as off-white solid. 1H NMR (300 MHz, DMSO-d6) δ:12.34 (s, 1H), 10.57 (s, 1H), 10.15 (s, 1H), 6.35 (s, 1H), 6.14-6.15 (m, 1H), 6.06-6.07 (m, 1H), 5.12-5.02 (m, 1H), 4.81 (s, 2H), 3.81 (s, 3H), 3.30-3.05 (m, 1H), 2.86-2.60 (m, 7H), 2.39-2.28 (m, 1H), 2.30-2.08 (m, 1H), 2.04-1.80 (m, 2H), 1.70-1.66 (m, 2H), 1.00-0.75 (m, 6H). MS (ESI) calcd. for C₂₄H₃₅N₅O₇, 503.24 m/z, found [M+H]⁺ 504.20 m/z.

### Example 33: Preparation of Compound 278

### Synthesis of (1R,3S)-3-(3-(2-(2-formyl-3-hydroxy-5-methoxyphenoxy)acetamido)-1H-pyrazol-5-yl)cyclopentyl 2-methylpyrazolidine-1-carboxylate

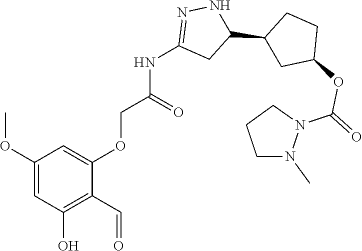 [SMILES: COc1cc(O)c(C=O)c(OCC(=O)NC2=NNC([C@H]3CC[C@@H](OC(=O)N4CCCN4C)C3)C2)c1]

### Synthetic Route

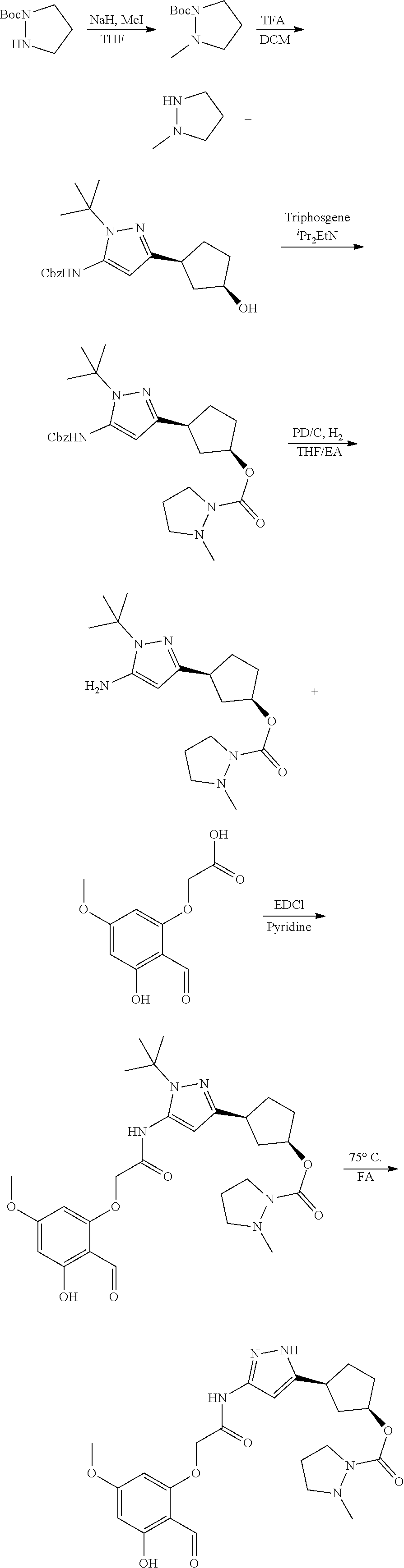

### Step 1: Synthesis of tert-butyl 2-methylpyrazolidine-1-carboxylate

To a stirred solution of tert-butyl pyrazolidine-1-carboxylate (800 mg, 4.645 mmol, 1 equiv.) in THF (10 mL) was added NaH (222.9 mg, 5.574 mmol, 1.2 equiv. 60% in oil) at 0° C. The resulting mixture was stirred at room temperature for 30 min. To the above mixture was added methyl iodide (659.31 mg, 4.645 mmol, 1 equiv.) at room temperature. The resulting mixture was stirred at room temperature overnight. The reaction was quenched with water at 0° C. the resulting mixture was extracted with DCM (3×40 mL). The combined organic layers were washed with brine (2×40 mL), dried over anhydrous Na₂SO₄, filtered, and concentrated under reduced pressure. The residue was purified by C18 reverse phase chromatography, 5 to 40% MeCN/H₂O with 5 mmol/L NH₄HCO₃ modifier to afford tert-butyl 2-methylpyrazolidine-1-carboxylate (300 mg, 35% yield) as a light yellow oil.

### Step 2: Synthesis of 1-methylpyrazolidine

To a stirred solution of tert-butyl 2-methylpyrazolidine-1-carboxylate (260 mg, 1.396 mmol, 1 equiv.) in DCM (2 mL) was added TFA (2 mL) at room temperature. The resulting mixture was stirred at room temperature for 2 h. The resulting mixture was concentrated under reduced atmosphere resulting in 1-methylpyrazolidine (110 mg, 91% yield) as a light yellow oil. The crude material was used without any further purification.

### Step 3: Synthesis of (1R,3S)-3-(5-(benzyloxycarbonylamino)-1-tert-butyl-1H-pyrazol-3-yl)cyclopentyl 2-methylpyrazolidine-1-carboxylate

To a stirred solution of benzyl N-{2-tert-butyl-5-[(1S,3R)-3-hydroxycyclopentyl]pyrazol-3-yl}carbamate (250 mg, 0.699 mmol, 1 equiv.) and triphosgene (415.06 mg, 1.398 mmol, 2 equiv.) in DCM (10 mL) was added DIEA (542.36 mg, 4.194 mmol, 6 equiv.) at 0° C. under nitrogen atmosphere. The resulting mixture was stirred at room temperature for 1 h under nitrogen atmosphere and subsequently concentrated under reduced atmosphere. To a stirred solution of 1-methylpyrazolidine (106.67 mg, 1.238 mmol, 2 equiv.) and DIEA (480.16 mg, 3.714 mmol, 6 equiv.) in THF (8 mL) was added to the above mixture at room temperature and left to stir overnight. The resulting mixture was quenched with water and extracted with EtOAc (3×20 mL). The combined organic layers were washed with brine (2×20 mL), dried over anhydrous Na₂SO₄, filtered, and concentrated under reduced pressure. The residue was purified by C18 reverse phase chromatography, 5% to 50% MeCN/H₂O with 5 mmol/L NH₄HCO₃ modifier to afford (1R,3S)-3-(5-(benzyloxycarbonylamino)-1-tert-butyl-1H-pyrazol-3-yl)cyclopentyl 2-methylpyrazolidine-1-carboxylate (210 mg, 72% yield) as a light yellow solid.

### Step 4: Synthesis of (1R,3S)-3-(5-amino-1-tert-butyl-1H-pyrazol-3-yl)cyclopentyl 2-methylpyrazolidine-1-carboxylate

To a solution of (1R,3S)-3-(5-(benzyloxycarbonylamino)-1-tert-butyl-1H-pyrazol-3-yl)cyclopentyl 2-methylpyrazolidine-1-carboxylate (210 mg, 0.447 mmol, 1 equiv.) in THF (3 mL) and EtOAc (3 mL) was added 10% Pd/C (300 mg). The mixture was maintain under a hydrogen atmosphere at room temperature for 1 h. The resulting mixture was filtered, the filter cake was washed with DCM (2×20 mL) and the filtrate was concentrated under reduced atmosphere. This resulted in (1R,3S)-3-(5-amino-1-tert-butyl-1H-pyrazol-3-yl)cyclopentyl 2-methylpyrazolidine-1-carboxylate (120 mg, 80% yield) as a brown yellow oil. The crude material was used without any further purification.

### Step 5: Synthesis of (1R,3S)-3-(1-tert-butyl-5-(2-(2-formyl-3-hydroxy-5-methoxyphenoxy)acetamido)-1H-pyrazol-3-yl)cyclopentyl 2-methylpyrazolidine-1-carboxylate

To a stirred solution of (1R,3S)-3-(5-amino-1-tert-butyl-1H-pyrazol-3-yl)cyclopentyl 2-methylpyrazolidine-1-carboxylate (110 mg, 0.328 mmol, 1 equiv.) and Intermediate A (133.50 mg, 0.590 mmol, 1.8 equiv.) in pyridine (4 mL) was added EDCI (138.30 mg, 0.722 mmol, 2.2 equiv.) left for 4 h. The resulting mixture was diluted with water and extracted with EtOAc (3×20 mL). The combined organic layers were washed with brine (2×20 mL), dried over anhydrous Na₂SO₄, filtered, and concentrated under reduced pressure. This resulted in (1R,3S)-3-(1-tert-butyl-5-(2-(2-formyl-3-hydroxy-5-methoxyphenoxy)acetamido)-1H-pyrazol-3-yl)cyclopentyl 2-methylpyrazolidine-1-carboxylate (100 mg, 56% yield) as a brown yellow oil. The crude material was used without any further purification.

### Step 6: Synthesis of (1R,3S)-3-(3-(2-(2-formyl-3-hydroxy-5-methoxyphenoxy)acetamido)-1H-pyrazol-5-yl)cyclopentyl 2-methylpyrazolidine-1-carboxylate

A solution of (1R,3S)-3-{1-tert-butyl-5-[2-(2-formyl-3-hydroxy-5-methoxyphenoxy)acetamido]pyrazol-3-yl}cyclopentyl 2-methylpyrazolidine-1-carboxylate (100 mg, 0.184 mmol, 1 equiv.) in formic acid (7 mL) was stirred at 75° C. for 6 h. The resulting mixture was concentrated under vacuum. The residue was purified by C18 reverse phase chromatography, 5 to 50% MeCN/H₂O with 0.05% TFA modifier to afford (1R,3S)-3-(3-(2-(2-formyl-3-hydroxy-5-methoxyphenoxy)acetamido)-1H-pyrazol-5-yl)cyclopentyl 2-methylpyrazolidine-1-carboxylate; trifluoroacetic acid (Compound 278, 33.9 mg, 31% yield) as a light yellow solid. MS (ESI) calcd. for C₂₃H₂₉N₅O₇ 487.21 m/z, found [M+H]⁺ 488.25 m/z. 1H NMR (300 MHz, DMSO-d6) δ (ppm): 12.25 (br s, 1H), 10.53 (s, 1H), 10.08 (s, 1H), 6.27 (s, 1H), 6.07-6.01 (m, 2H), 5.05-5.03 (m, 1H), 4.75 (s, 2H), 3.74 (s, 3H) 3.50-3.43 (m, 2H), 3.21-3.04 (m, 3H), 2.64 (s, 3H), 2.40-2.34 (m, 1H), 2.08-1.95 (m, 3H), 1.90-1.78 (m, 2H), 1.73-1.61 (m, 2H).

### Example 34: Preparation of Compound 279

### Synthesis of (1R,3S)-3-(3-(2-(2-formyl-3-hydroxy-5-methoxyphenoxy)acetamido)-1H-pyrazol-5-yl)cyclopentyl 1,2-dimethylhydrazine-1-carboxylate

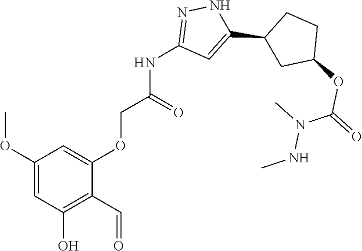 [SMILES: CNN(C)C(=O)O[C@@H]1CC[C@H](c2cc(NC(=O)COc3cc(OC)cc(O)c3C=O)n[nH]2)C1]

### Synthetic Route

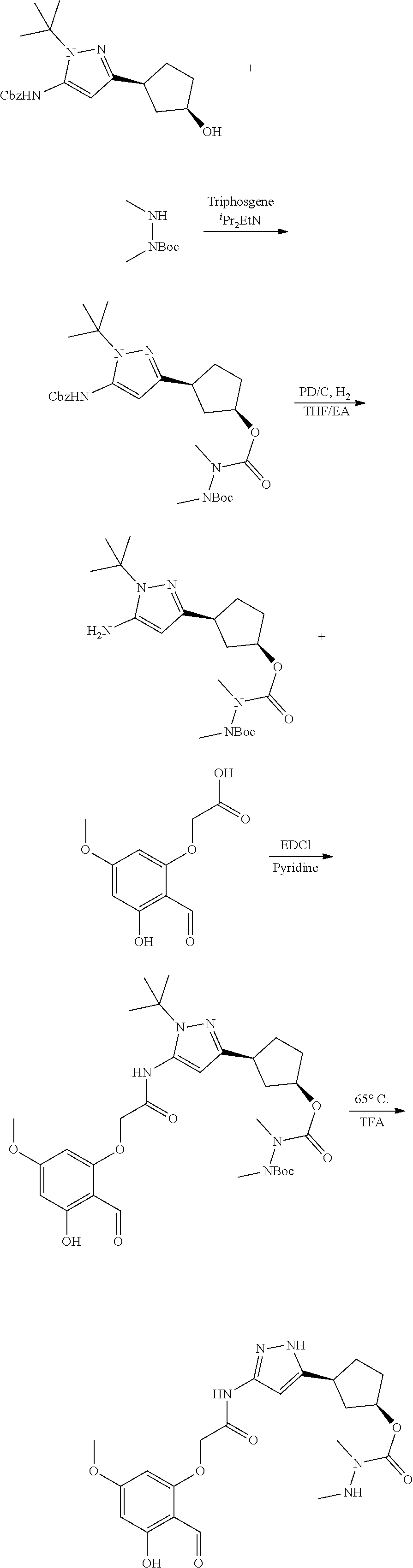

### Step 1: Synthesis of 1-((1R,3S)-3-(5-(((benzyloxy)carbonyl)amino)-1-(tert-butyl)-1H-pyrazol-3-yl)cyclopentyl) 2-(tert-butyl) 1,2-dimethylhydrazine-1,2-dicarboxylate

A solution of benzyl (1-(tert-butyl)-3-((1S,3R)-3-hydroxycyclopentyl)-1H-pyrazol-5-yl)carbamate (1.00 g, 2.798 mmol, 1.00 equiv.) in DCM (20 mL) was treated with triphosgene (1.66 g, 5.596 mmol, 2 equiv.) at room temperature followed by the addition of DIEA (2.17 g, 16.788 mmol, 6 equiv.) at 0° C. The resulting mixture was stirred at room temperature for 1 h under nitrogen atmosphere then concentrated under reduced pressure. The remaining residue was redissolved in THF (20 mL) to which was added tert-butyl 1,2-dimethylhydrazine-1-carboxylate (0.672 g, 4.196 mmol, 1.5 equiv.), DIEA (2.17 g, 16.788 mmol, 6 equiv.) at room temperature and allowed to stir for 2 h. The reaction was quenched with water at room temperature and extracted with EtOAc (3×60 mL). The combined organic layers were washed with brine (2×60 mL), dried over anhydrous Na₂SO₄, filtered, and concentrated under reduced pressure. The crude material was purified by C18 reverse phase chromatography, 5 to 50% MeCN/H₂O to afford 1-((1R,3S)-3-(5-(((benzyloxy)carbonyl)amino)-1-(tert-butyl)-1H-pyrazol-3-yl)cyclopentyl) 2-(tert-butyl) 1,2-dimethylhydrazine-1,2-dicarboxylate (680 mg, 44% yield) as a white solid.

### Step 2: Synthesis of 1-((1R,3S)-3-(5-amino-1-(tert-butyl)-1H-pyrazol-3-yl)cyclopentyl) 2-(tert-butyl) 1,2-dimethylhydrazine-1,2-dicarboxylate

To a stirred solution of 1-((1R,3S)-3-(5-(((benzyloxy)carbonyl)amino)-1-(tert-butyl)-1H-pyrazol-3-yl)cyclopentyl) 2-(tert-butyl) 1,2-dimethylhydrazine-1,2-dicarboxylate (680 mg, 1.395 mmol, 1 equiv.) in EtOAc (8 mL) and THF (8 mL) was added Pd/C (750 mg) at room temperature. The resulting mixture was stirred at room temperature for 2 h under hydrogen atmosphere. The reaction mixture was then diluted with DCM (20 mL), filtered, the filter cake was washed with DCM (3×20 mL) and the filtrate was concentrated under reduced pressure. This resulted in 1-((1R,3S)-3-(5-amino-1-(tert-butyl)-1H-pyrazol-3-yl)cyclopentyl) 2-(tert-butyl) 1,2-dimethylhydrazine-1,2-dicarboxylate (500 mg, 97% yield) as a white solid. The crude product was used in the next step directly without further purification.

### Step 3: Synthesis of 1-(tert-butyl) 2-((1R,3S)-3-(1-(tert-butyl)-5-(2-(2-formyl-3-hydroxy-5-methoxyphenoxy)acetamido)-1H-pyrazol-3-yl)cyclopentyl) 1,2-dimethylhydrazine-1,2-dicarboxylate

To a stirred solution of 1-((1R,3S)-3-(5-amino-1-(tert-butyl)-1H-pyrazol-3-yl)cyclopentyl) 2-(tert-butyl) 1,2-dimethylhydrazine-1,2-dicarboxylate (230 mg, 0.651 mmol, 1 equiv.) and Intermediate A (176.63 mg, 0.781 mmol, 1.2 equiv.) in pyridine (5 mL) was added EDCI (187.13 mg, 0.977 mmol, 1.5 equiv.) at room temperature. The resulting mixture was stirred at room temperature for 2 h. The reaction mixture was quenched with water at room temperature, and extracted with EtOAc (3×15 mL). The combined organic layers were washed with brine (3×15 mL), dried over anhydrous Na₂SO₄, filtered, and concentrated under reduced pressure. This resulted in 1-(tert-butyl) 2-((1R,3S)-3-(1-(tert-butyl)-5-(2-(2-formyl-3-hydroxy-5-methoxyphenoxy)acetamido)-1H-pyrazol-3-yl)cyclopentyl) 1,2-dimethylhydrazine-1,2-dicarboxylate (120 mg, 35% yield) as a white solid. The crude product was used in the next step directly without further purification.

### Step 4: Synthesis of (1R,3S)-3-(3-(2-(2-formyl-3-hydroxy-5-methoxyphenoxy)acetamido)-1H-pyrazol-5-yl)cyclopentyl 1,2-dimethylhydrazine-1-carboxylate

A solution of 1-(tert-butyl) 2-((1R,3S)-3-(1-(tert-butyl)-5-(2-(2-formyl-3-hydroxy-5-methoxyphenoxy)acetamido)-1H-pyrazol-3-yl)cyclopentyl) 1,2-dimethylhydrazine-1,2-dicarboxylate (100 mg, 0.178 mmol, 1 equiv.) in TFA (5 mL) was stirred at 65° C. for 4 h. The reaction mixture was cooled to room temperature and purified by C18 reverse phase chromatography, 5 to 50% MeCN/H₂O to afford (1R,3S)-3-(3-(2-(2-formyl-3-hydroxy-5-methoxyphenoxy)acetamido)-1H-pyrazol-5-yl)cyclopentyl 1,2-dimethylhydrazine-1-carboxylate (Compound 279, 29.4 mg, 39% yield) as a white solid. 1H NMR (300 MHz, DMSO-d6) δ 12.32 (s, 1H), 10.56 (s, 1H), 10.15 (s, 1H), 6.34 (s, 1H), 6.15-6.14 (m, 1H), 6.07-6.06 (m, 1H), 5.10-5.08 (m, 1H), 4.81 (s, 2H), 3.81 (s, 3H), 3.19-3.08 (m, 1H), 2.96 (s, 3H), 2.54-2.51 (m, 3H), 2.49-2.40 (m, 1H), 2.06-2.00 (m, 1H), 1.98-1.86 (m, 4H). MS (ESI) calcd. for C₂₁H₂₇N₅O₇ 461.19 m/z, found [M+H]⁺ 462.30 m/z.

### Example 35: Preparation of Compounds 280 and 281

### Synthesis of (1R,3S)-3-(3-(2-(2-formyl-3-hydroxy-5-methoxyphenoxy)acetamido)-1H-pyrazol-5-yl)cyclopentyl ((*)-2,2-difluoro-1-methylcyclopropyl)carbamate (Compound 280) and (1R,3S)-3-(3-(2-(2-formyl-3-hydroxy-5-methoxyphenoxy)acetamido)-1H-pyrazol-5-yl)cyclopentyl ((*)-2,2-difluoro-1-methylcyclopropyl)carbamate (Compound 281)

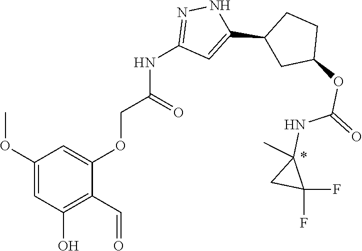 [SMILES: COc1cc(O)c(C=O)c(OCC(=O)Nc2cc([C@H]3CC[C@@H](OC(=O)NC4(C)CC4(F)F)C3)[nH]n2)c1]

### Synthetic Route

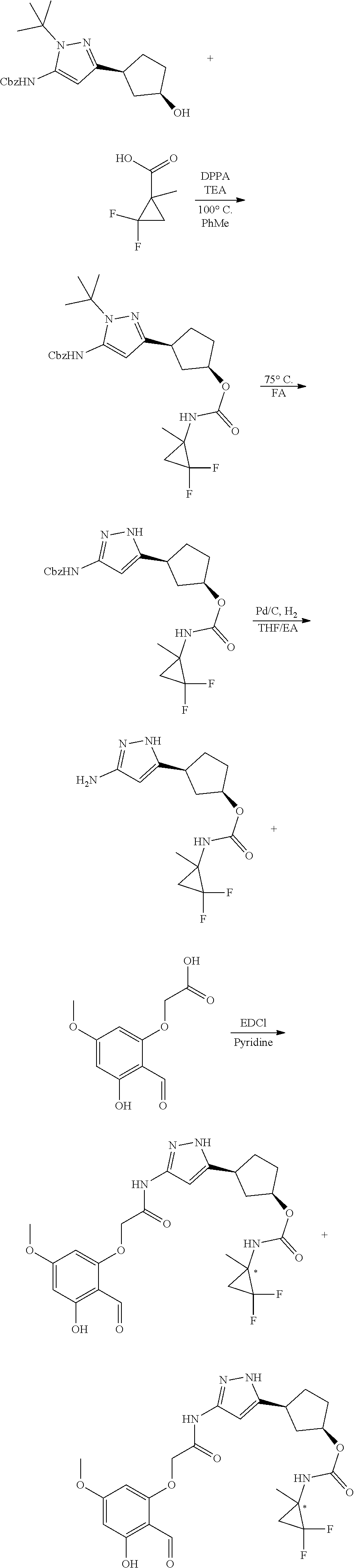

### Step 1: Synthesis of (1R,3S)-3-(5-(((benzyloxy)carbonyl)amino)-1-(tert-butyl)-1H-pyrazol-3-yl)cyclopentyl (2,2-difluoro-1-methylcyclopropyl)carbamate

To a stirred solution of benzyl N-{2-tert-butyl-5-[(1S,3R)-3-hydroxycyclopentyl]pyrazol-3-yl}carbamate (1.0 g, 2.798 mmol, 1 equiv.) and 2,2-difluoro-1-methylcyclopropane-1-carboxylic acid (1.14 g, 8.394 mmol, 3.0 equiv.) in toluene (20 mL) was added DPPA (4.62 g, 16.788 mmol, 6.0 equiv.) and TEA (2.12 g, 20.985 mmol, 7.5 equiv.) at room temperature then heated to 100° C. for 3 h under N₂ atmosphere. The resulting mixture was cooled to room temperature, diluted with water, and extracted with EtOAc (3×50 mL). The combined organic layers were washed with brine (2×20 mL), dried over anhydrous Na₂SO₄, filtered, and concentrated under reduced pressure. The crude material was diluted with DMF and water, filtered through a syringe filter and purified by C18 reverse phase chromatography, 10 to 70% MeCN/H₂O with 0.05% TFA modifier to afford (1R,3S)-3-(5-{[(benzyloxy)carbonyl]amino}-1-tert-butylpyrazol-3-yl)cyclopentyl N-(2,2-difluoro-1-methylcyclopropyl)carbamate (0.55 g, 52.38% yield) as a pink solid.

### Step 2: Synthesis of (1R,3S)-3-(5-(((benzyloxy)carbonyl)amino)-1H-pyrazol-3-yl)cyclopentyl (2,2-difluoro-1-methylcyclopropyl)carbamate

(1R,3S)-3-(5-{[(benzyloxy)carbonyl]amino}-1-tert-butylpyrazol-3-yl)cyclopentyl N-(2,2-difluoro-1-methylcyclopropyl)carbamate (0.5 g, 1.019 mmol, 1 equiv.) was dissolved in formic acid (10 mL) at room temperature. Then the resulting mixture was stirred at 75° C. for 5 h. The resulting mixture was cooled to room temperature and concentrated under reduced pressure. The crude material was diluted with DMF and water, filtered through a syringe filter and purified by C18 reverse phase chromatography, 10 to 70% MeCN/H₂O with 0.05% TFA modifier to afford (1R,3S)-3-(5-{[(benzyloxy)carbonyl]amino}-1H-pyrazol-3-yl)cyclopentyl N-(2,2-difluoro-1-methylcyclopropyl)carbamate (0.36 g, 81.82% yield) as a white solid.

### Step 3: Synthesis of (1R,3S)-3-(5-amino-1H-pyrazol-3-yl)cyclopentyl (2,2-difluoro-1-methylcyclopropyl)carbamate

To a stirred solution of (1R,3S)-3-(5-{[(benzyloxy)carbonyl]amino}-1H-pyrazol-3-yl)cyclopentyl N-(2,2-difluoro-1-methylcyclopropyl)carbamate (0.4 g, 0.921 mmol, 1 equiv.) in EtOAc (5 mL) and THF (5 mL) was added 10% Pd/C (0.20 g) at room temperature. The resulting mixture was stirred at room temperature for 2 h under H₂ atmosphere. The resulting mixture was filtered, the filter cake was washed with MeOH (3×10 mL). The filtrate was concentrated under reduced pressure to afford (1R,3S)-3-(5-amino-1H-pyrazol-3-yl)cyclopentyl N-(2,2-difluoro-1-methylcyclopropyl)carbamate (0.2 g, 60.97% yield) as a brown solid. The crude material was used without any further purification.

### Step 4: Synthesis of (1R,3S)-3-(3-(2-(2-formyl-3-hydroxy-5-methoxyphenoxy)acetamido)-1H-pyrazol-5-yl)cyclopentyl ((*)-2,2-difluoro-1-methylcyclopropyl)carbamate and (1R,3S)-3-(3-(2-(2-formyl-3-hydroxy-5-methoxyphenoxy)acetamido)-1H-pyrazol-5-yl)cyclopentyl ((*)-2,2-difluoro-1-methylcyclopropyl)carbamate

To a stirred solution of (1R,3S)-3-(5-amino-1H-pyrazol-3-yl)cyclopentyl N-(2,2-difluoro-1-methylcyclopropyl)carbamate (50 mg, 0.166 mmol, 1 equiv.) and 2-formyl-3-hydroxy-5-methoxyphenoxyacetic acid (37.66 mg, 0.166 mmol, 1.0 equiv.) in pyridine (3 mL) was added EDCI (38.77 mg, 0.249 mmol, 1.5 equiv.) and stirred for 2 h at room temperature. The resulting mixture was diluted with water and extracted with EtOAc (3×20 mL). The combined organic layers were washed with brine (2×20 mL), dried over anhydrous Na₂SO₄, filtered, and concentrated under reduced pressure. The crude material was diluted with DMF and water, filtered through a syringe filter and purified by prep-HPLC, 10 to 70% MeCN/H₂O with 0.05% TFA modifier followed by Chiral-HPLC to afford (1R,3S)-3-(3-(2-(2-formyl-3-hydroxy-5-methoxyphenoxy)acetamido)-1H-pyrazol-5-yl)cyclopentyl ((*)-2,2-difluoro-1-methylcyclopropyl)carbamate (Compound 280, 4.8 mg, 5.67% yield) as a white solid. 1H NMR (300 MHz, DMSO-d6) δ 12.33 (s, 1H), 10.57 (s, 1H), 10.15 (s, 1H), 7.72-7.44 (m, 1H), 6.31 (s, 1H), 6.15 (s, 1H), 6.07 (s, 1H), 5.02-4.97 (m, 1H), 4.81 (s, 2H), 3.81 (s, 3H), 3.08-3.04 (m, 1H), 2.51-2.42 (m, 1H), 2.08-1.89 (m, 2H), 1.73-1.51 (m, 5H), 1.45-1.30 (m, 3H). MS (ESI) calcd. for C₂₃H₂₆F₂N₄O₇, 508.18 m/z, found [M+H]⁺ 509.20 m/z, and (1R,3S)-3-(3-(2-(2-formyl-3-hydroxy-5-methoxyphenoxy)acetamido)-1H-pyrazol-5-yl)cyclopentyl ((*)-2,2-difluoro-1-methylcyclopropyl)carbamate (Compound 281, 6.6 mg, 7.80% yield) as a white solid. 1H NMR (300 MHz, DMSO-d6) δ 12.33 (s, 1H), 10.57 (s, 1H), 10.15 (s, 1H), 7.72-7.43 (m, 1H), 6.31 (s, 1H), 6.15 (s, 1H), 6.07 (s, 1H), 5.02-4.97 (m, 1H), 4.81 (s, 2H), 3.81 (s, 3H), 3.07-3.03 (m, 1H), 2.50-2.49 (m, 1H), 2.02-1.88 (m, 2H), 1.75-1.65 (m, 2H), 1.60-1.54 (m, 3H), 1.35-1.30 (m, 3H). MS (ESI) calcd. for C₂₃H₂₆F₂N₄O₇, 508.18 m/z, found [M+H]⁺ 509.15 m/z.

### Example 36: Preparation of Compound 282

### Synthesis of (1R,3s)-3-(3-((*)-4-formyl-5-hydroxy-7-methoxy-2,3-dihydro-1H-indene-2-carboxamido)-1H-pyrazol-5-yl)cyclobutyl isopropylcarbamate

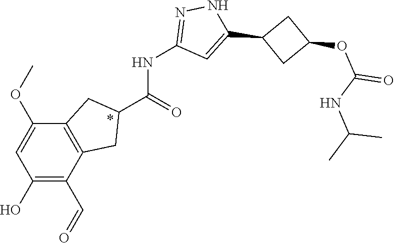 [SMILES: COc1cc(O)c(C=O)c2c1CC(C(=O)Nc1cc([C@H]3C[C@@H](OC(=O)NC(C)C)C3)[nH]n1)C2]

### Synthetic Route

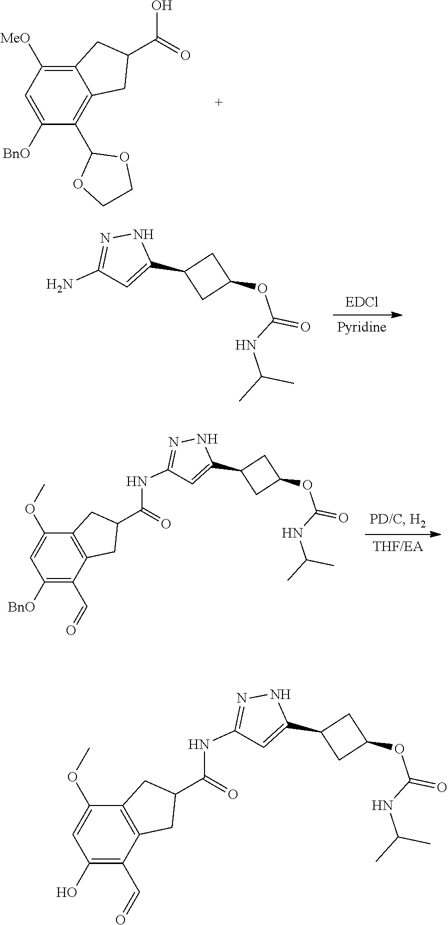

### Step 1: (1s,3s)-3-(3-(5-(benzyloxy)-4-formyl-7-methoxy-2,3-dihydro-1H-indene-2-carboxamido)-1H-pyrazol-5-yl)cyclobutyl isopropylcarbamate

(1s,3s)-3-(3-amino-1H-pyrazol-5-yl)cyclobutyl isopropylcarbamate (500 mg, 2.098 mmol), 5-(benzyloxy)-4-(1,3-dioxolan-2-yl)-7-methoxy-2,3-dihydro-1H-indene-2-carboxylic acid (932.6 g, 2.518 mmol), pyridine (20 mL) and a stir bar were added to an nitrogen-purged 100 mL round-bottom and stirred until homogenous, then treated with EDCI (651 g, 4.196 mmol) and lef to stir at room temperature for 2 h. The reaction mixture was quenched with water, extracted 3 times with 30 mL EA. The organic layers were combined, dried over Na₂SO₄, filtered, and concentrated under reduced atmosphere. The resulting crude material was resuspended in FA (18 mL) and H₂O (6 mL) and stirred at room temperature for 2 h. The reaction mixture was purified by reverse-phase chromatography, 5 to 50% MeCN/H₂O with 0.05% TFA modifier to afford (1s,3s)-3-(3-(5-(benzyloxy)-4-formyl-7-methoxy-2,3-dihydro-1H-indene-2-carboxamido)-1H-pyrazol-5-yl)cyclobutyl isopropylcarbamate (330 mg, 26.63% yield) as a white solid MS (ESI) mass calcd. for C₃₀H₃₄N₄O₆, 546.25 m/z, found 547.25 [M+H]⁺.

### Step 2: (1R,3s)-3-(3-((*)-4-formyl-5-hydroxy-7-methoxy-2,3-dihydro-1H-indene-2-carboxamido)-1H-pyrazol-5-yl)cyclobutyl isopropylcarbamate

(1s,3s)-3-(3-(5-(benzyloxy)-4-formyl-7-methoxy-2,3-dihydro-1H-indene-2-carboxamido)-1H-pyrazol-5-yl)cyclobutyl isopropylcarbamate (310 mg, 0.567 mmol), Pd/C (155 mg, 1.457 mmol, 10%), a stir bar, EA (5 mL) and THF (5 mL) were added to a nitrogen-purged 50 mL round-bottom. The round-bottom was evacuated and flushed three times with nitrogen, followed by flushing with hydrogen. The reaction mixture was maintained under H₂ and stirred at room temperature for 2 h, then the mixture was filtered through a celite pad and washed with EA (30 mL). The filtrate was concentrated under reduced atmosphere and purified by reverse-phase chromatography, 5 to 50% MeCN/H₂O with 0.05% TFA modifier to afford a crude product which was further purified by chiral HPLC; Column: CHIRALPAK IG, 2×25 cm, 5 μm; Mobile Phase A: HEX (0.1% FA), Mobile Phase B: EtOH:DCM=1:1—HPLC; Flow rate: 20 mL/min; Gradient (B %): isocratic 90; Wave Length: 220/254 nm; RT1 (min): 6.05; RT2 (min): 7.789; Sample Solvent: EtOH; Injection Volume: 0.5 mL to afford (1R,3s)-3-(3-((*)-4-formyl-5-hydroxy-7-methoxy-2,3-dihydro-1H-indene-2-carboxamido)-1H-pyrazol-5-yl)cyclobutyl isopropylcarbamate) (Compound 282, 52.3 mg, 99.2% purity) as a white solid. MS (ESI) mass calcd. for C₂₃H₂₈N₄O₆, 456.20 m/z, found 457.20 [M+H]⁺. 1H NMR (400 MHz, DMSO-d6) δ 11.23 (s, 1H), 10.46 (s, 1H), 10.06 (s, 1H), 7.07 (d, J=7.8 Hz, 1H), 6.39 (s, 1H), 6.33 (s, 1H), 4.79 (p, J=7.5 Hz, 1H), 3.62-3.52 (m, 1H), 3.52-3.35 (m, 2H), 3.34-3.20 (m, 1H), 3.10-3.01 (m, 1H), 3.01-2.91 (m, 1H), 2.88-2.79 (m, 1H), 2.70-2.61 (m, 2H), 2.10-1.98 (m, 2H), 1.30-1.12 (m, 3H), 1.03 (d, J=6.5 Hz, 6H). * Denotes a stereocenter with an undetermined absolute stereochemistry of a single isomer.

### Example 37: Preparation of Compound 283

### Synthesis of (1R,3S)-3-(3-((*)-4-formyl-5-hydroxy-7-methoxy-2,3-dihydro-1H-indene-2-carboxamido)-1H-pyrazol-5-yl)cyclopentyl tert-butylcarbamate (Compound 283)

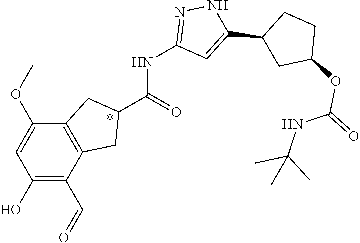 [SMILES: COc1cc(O)c(C=O)c2c1CC(C(=O)Nc1cc([C@H]3CC[C@@H](OC(=O)NC(C)(C)C)C3)[nH]n1)C2]

### Synthetic Route

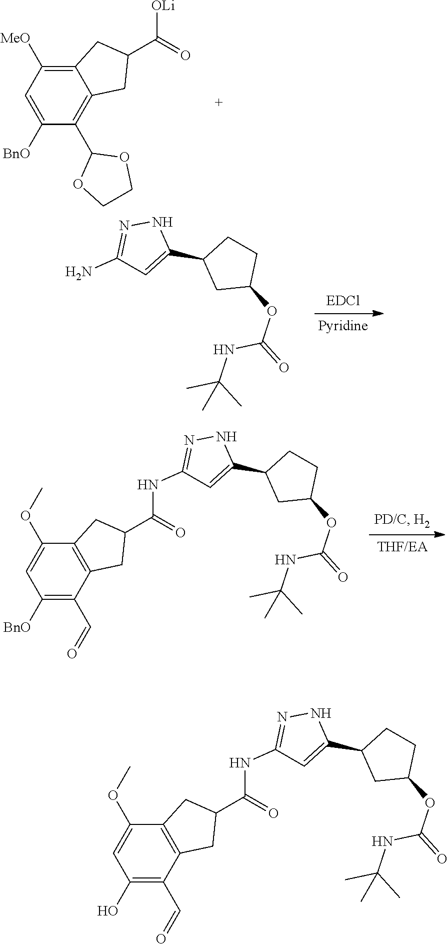

### Step 1: (1R,3S)-3-(3-(5-(benzyloxy)-4-formyl-7-methoxy-2,3-dihydro-1H-indene-2-carboxamido)-1H-pyrazol-5-yl)cyclopentyl tert-butylcarbamate

5-(Benzyloxy)-4-(1,3-dioxolan-2-yl)-7-methoxy-2,3-dihydro-1H-indene-2-carboxylic acid (1.1 g, 2.970 mmol), (1R,3S)-3-(3-amino-1H-pyrazol-5-yl)cyclopentyl tert-butylcarbamate (0.95 g, 3.564 mmol), pyridine (10 mL) and a stir bar were added to a nitrogen-purged 50 mL round-bottom and stirred until homogenous, then treated with EDCI (0.69 g, 4.455 mmol). The reaction mixture was stirred at room temperature for 2 h, then quenched with water. The mixture was extracted 3 times with 15 mL EA. The organic layers were combined, dried over Na₂SO₄, filtered, and concentrated to dryness in vacuo. The resulting crude material was resuspended in FA (18 mL) and H₂O (6 mL) and stirred at room temperature for 2 h. The resulting mixture was purified by reverse-phase chromatography, 5 to 50% MeCN/H₂O with 0.05% TFA modifier to afford (1R,3S)-3-(3-(5-(benzyloxy)-4-formyl-7-methoxy-2,3-dihydro-1H-indene-2-carboxamido)-1H-pyrazol-5-yl)cyclopentyl tert-butylcarbamate (370 mg, 21.7% yield) as a white solid.

### Step 2: (1R,3S)-3-(3-((*)-4-formyl-5-hydroxy-7-methoxy-2,3-dihydro-1H-indene-2-carboxamido)-1H-pyrazol-5-yl)cyclopentyl tert-butylcarbamate

(1R,3S)-3-(3-(5-(benzyloxy)-4-formyl-7-methoxy-2,3-dihydro-1H-indene-2-carboxamido)-1H-pyrazol-5-yl)cyclopentyl tert-butylcarbamate (370.0 mg, 0.644 mmol), Pd/C (185 g, 1.738 mmol, 10%), a stir bar, EA (2 mL) and THF (2 mL) were added to a nitrogen-purged 100 mL round-bottom. The round-bottom was evacuated and flushed three times with nitrogen, followed by flushing with hydrogen. The reaction mixture was maintained under H₂ and stirred overnight at room temperature, the mixture was then filtered through a celite pad and washed with EA (10 mL). The filtrate was concentrated under vacuum to afford the crude product. The material product was further purified by chrial HPLC; Column: CHIRALPAK IG, 2×25 cm, 5 μm; Mobile Phase A: HEX (0.2% DEA)—HPLC, Mobile Phase B: EtOH:DCM=1:1—HPLC; Flow rate: 20 mL/min; Gradient (B %): isocratic 70; Wave Length: 220/254 nm; RT1 (min): 8.133; RT2 (min): 11.12; Sample Solvent: EtOH—HPLC; Injection Volume: 1.0 mL to afford (1R,3S)-3-(3-((*)-4-formyl-5-hydroxy-7-methoxy-2,3-dihydro-1H-indene-2-carboxamido)-1H-pyrazol-5-yl)cyclopentyl tert-butylcarbamate (Compound 283, 36.8 mg, 98.0% purity) as a white solid. MS (ESI) mass calcd. for C₂₅H₃₂N₄O₆, 484.55 m/z, found 485.250 [M+H]⁺. 1H NMR (400 MHz, DMSO-d6) δ 12.06 (s, 1H), 11.23 (s, 1H), 10.44 (s, 1H), 10.06 (s, 1H), 6.78 (s, 1H), 6.39 (s, 1H), 6.33 (s, 1H), 5.02-4.90 (m, 1H), 3.83 (s, 3H), 3.52-3.38 (m, 2H), 3.30-3.20 (m, 1H), 3.10-2.89 (m, 2H), 2.87-2.78 (m, 1H), 2.48-2.40 (m, 1H), 2.04-1.93 (m, 1H), 1.93-1.79 (m, 1H), 1.76-1.62 (m, 2H), 1.62-1.50 (m, 1H), 1.23 (s, 1H), 1.19 (s, 9H). * Denotes a stereocenter with an undetermined absolute stereochemistry of a single isomer.

### Example 38: Preparation of Compound 284

### Synthesis of (1R,3S)-3-(3-((*)-4-formyl-5-hydroxy-7-methoxy-2,3-dihydro-1H-indene-2-carboxamido)-1H-pyrazol-5-yl)cyclopentyl 1,2,2-trimethylhydrazine-1-carboxylate (Compound 284)

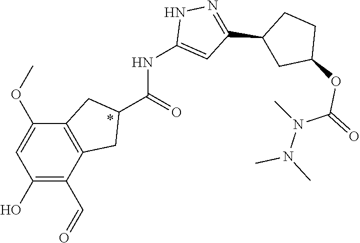 [SMILES: COc1cc(O)c(C=O)c2c1CC(C(=O)Nc1cc([C@H]3CC[C@@H](OC(=O)N(C)N(C)C)C3)n[nH]1)C2]

### Synthetic Route

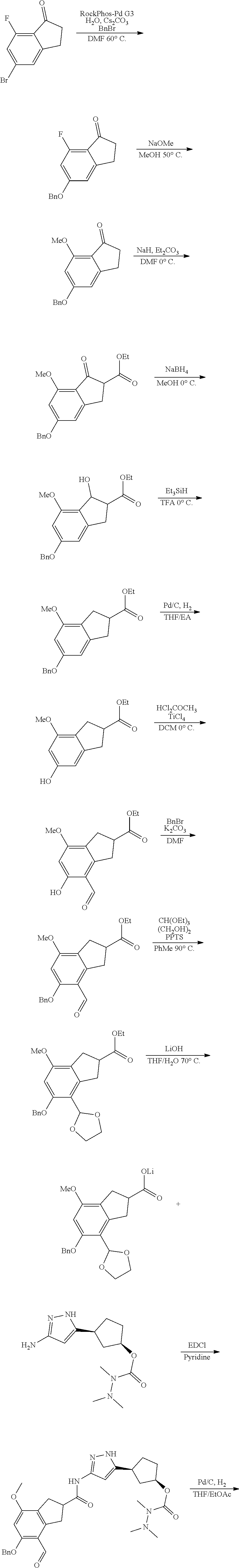

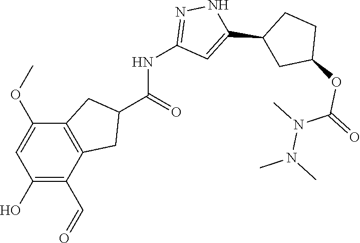 [SMILES: COc1cc(O)c(C=O)c2c1CC(C(=O)Nc1cc([C@H]3CC[C@@H](OC(=O)N(C)N(C)C)C3)[nH]n1)C2]

### Step 1: 5-(benzyloxy)-7-fluoro-2,3-dihydro-1H-inden-1-one

5-Bromo-7-fluoro-2,3-dihydroinden-1-one (192 g, 838.252 mmol, 1 equiv.), Cs₂CO₃ (682.8 g, 2095.642 mmol, 2.50 equiv.), di-tert-butyl[3-methoxy-6-methyl-2′,4′,6′-tris(propan-2-yl)-[1,1′-biphenyl]-2-yl]phosphane; {2′-amino-[1,1′-biphenyl]-2-yl}palladio methanesulfonate (3.5 g, 4.174 mmol, 0.005 equiv.), water (160 mL), a stir bar and DMF (1600 mL) were added to a 3 L 3-necked round bottom flask. The mixture was stirred for 1.5 h at 60° C. under nitrogen atmosphere. To the above mixture was added benzyl bromide (172 g, 1005.630 mmol, 1.20 equiv.) at r.t. The resulting mixture was stirred for 1.5 h at 60° C., then filtered. The resulting mixture was diluted with EtOAc and washed with H₂O and brine. The organic layers were dried over sodium sulphate, filtered, and concentrated to dryness under reduced atmosphere. The residue was purified by silica gel chromatography, 0 to 30% EA/PE to afford a crude product that was triturated with a mixture of EA and PE (1/7) and filtered to afford 5-(benzyloxy)-7-fluoro-2,3-dihydroinden-1-one (210 g, 747.31 mmol, 89% yield).

### Step 2: 5-(benzyloxy)-7-methoxy-2,3-dihydro-1H-inden-1-one

5-(Benzyloxy)-7-fluoro-2,3-dihydroinden-1-one (210 g, 747.319 mmol, 1 equiv., 91.2%), a stir bar and methanol (1600 mL) were added to a 5 L round bottom flask, then treated with sodium methoxide (800 mL). The resulting mixture was stirred at 50° C. for 2 h. Fifty percent of the solvent was removed under vacuum. The mixture was added to a mixture of ice and water, then extracted with EA. The organic layers were dried over sodium sulphate, filtered, and concentrated to dryness under reduced atmosphere. The residue obtained was purified by silica gel chromatography, 0 to 35% EA/PE to afford 5-(benzyloxy)-7-methoxy-2,3-dihydroinden-1-one (180 g, 617.86 mmol, 82% yield).

### Step 3: ethyl 5-(benzyloxy)-7-methoxy-1-oxo-2,3-dihydro-1H-indene-2-carboxylate

5-(Benzyloxy)-7-methoxy-2,3-dihydroinden-1-one (180 g, 617.863 mmol, 1 equiv., 92.1%), a stir bar and DMF (1400 mL) were added to a 3000 mL 3-necked round bottom flask, then treated with NaH (37.0 g, 925.077 mmol, 1.50 equiv., 60%) in portions under N₂ at 0° C. After stirring for 30 min, diethyl carbonate (109.5 g, 926.929 mmol, 1.50 equiv.) was added at 0° C. The mixture was stirred at r.t for 2 h, then quenched with a saturated solution of NH₄Cl. The mixture was diluted with EA and washed with H₂O and brine. The organic layers were dried over sodium sulphate, filtered and concentrated to dryness in vacuo. The residue obtained was triturated with a mixture of Et₂O and hexane (4/1) and filtered to afford ethyl 5-(benzyloxy)-7-methoxy-1-oxo-2,3-dihydroindene-2-carboxylate (207.6 g, 470.58 mmol, 76% yield).

### Step 4: ethyl 5-(benzyloxy)-1-hydroxy-7-methoxy-2,3-dihydro-1H-indene-2-carboxylate

Ethyl 5-(benzyloxy)-7-methoxy-1-oxo-2,3-dihydroindene-2-carboxylate (203.6 g, 461.184 mmol, 1 equiv., 77.1%), a stir bar and MeOH (2000 mL) were added to a 5000 mL flask, then treated with NaBH₄ (26.2 g, 692.572 mmol, 1.50 equiv.) in portions at 0° C. The resulting mixture was stirred at 0° C. for 1 h. The reaction was quenched with a saturated solution of NH₄Cl and extracted with EA. The organic layers were dried over sodium sulphate, filtered, and concentrated to dryness in vacuo to afford ethyl 5-(benzyloxy)-1-hydroxy-7-methoxy-2,3-dihydro-1H-indene-2-carboxylate (198 g, 400.10 mmol, 86% yield). The crude material was used without any further purification.

### Step 5: ethyl 6-(benzyloxy)-4-methoxy-2,3-dihydro-1H-indene-2-carboxylate

Ethyl 5-(benzyloxy)-1-hydroxy-7-methoxy-2,3-dihydro-1H-indene-2-carboxylate (178 g, 358.71 mmol, 69%), a stir bar and TFA (400 mL) were added to a 2000 mL flask, and treated with triethylsilane (400 mL). The resulting mixture was stirred at r.t for 1 h, then concentrated under reduced pressure. The obtained residue was diluted with DCM and the pH of the solution was adjusted to 7-8 with NaHCO₃ solution, then extracted with DCM. The organic layers were dried over sodium sulphate, filtered and concentrated to dryness in vacuo. The residue obtained was purified by silica gel chromatography, 0 to 35% EA/PE to afford ethyl 6-(benzyloxy)-4-methoxy-2,3-dihydro-1H-indene-2-carboxylate (96.7 g, 233.33 mmol, 44% yield).

### Step 6: ethyl 6-hydroxy-4-methoxy-2,3-dihydro-1H-indene-2-carboxylate

Ethyl 6-(benzyloxy)-4-methoxy-2,3-dihydro-1H-indene-2-carboxylate (106.7 g, 257.276 mmol, 78.7%), a stir bar, EA (500 mL) and THF (500 mL) were added to a 2000 mL flask, then treated with Pd/C (43.81 g, 41.164 mmol, 0.16 equiv., 10%). The resulting mixture was maintained under H₂ atmosphere and stirred at r.t for 3 h, then filtered and concentrated to dryness in vacuo. The residue obtained was purified by silica gel chromatography, 0 to 35% EA/PE to afford ethyl 6-hydroxy-4-methoxy-2,3-dihydro-1H-indene-2-carboxylate (56.5 g, 213.21 mmol, 82% yield).

### Step 7: ethyl 4-formyl-5-hydroxy-7-methoxy-2,3-dihydro-1H-indene-2-carboxylate

Ethyl 6-hydroxy-4-methoxy-2,3-dihydro-1H-indene-2-carboxylate (54.5 g, 205.297 mmol, 1 equiv., 89%), a stir bar and DCM (500 mL) were added to a 2000 mL 3-necked round bottom flask and stirred until homogenous, then treated with titanium tetrachloride (313 mL, 313.000 mmol, 1.50 equiv.) at 0° C. After stirring for 20 min, a mixture of dichloro(methoxy)methane (47 g, 408.873 mmol, 1.99 equiv.) in DCM (100 mL) and titanium tetrachloride (313 mL, 313.000 mmol, 1.50 equiv.) were added dropwise. The resulting mixture was stirred at 0° C. for 1 h. The reaction was quenched with H₂O and extracted with DCM. The organic layers were dried over sodium sulphate, filtered, and concentrated to dryness in vacuo to afford ethyl 4-formyl-5-hydroxy-7-methoxy-2,3-dihydro-1H-indene-2-carboxylate (61 g, 180.03 mmol, 87% yield). The crude material was used without any further purification.

### Step 8: ethyl 5-(benzyloxy)-4-formyl-7-methoxy-2,3-dihydro-1H-indene-2-carboxylate

Ethyl 4-formyl-5-hydroxy-7-methoxy-2,3-dihydro-1H-indene-2-carboxylate (61 g, 180.038 mmol, 1 equiv., 78%), K₂CO₃ (49.8 g, 360.334 mmol, 2.00 equiv.), a stir bar and DMF (500 mL) were added to a 1000 mL flask. After stirring for 30 min at r.t, benzyl bromide (40.0 g, 233.868 mmol, 1.30 equiv.) was added. The reaction was stirred for 2 h at room temperature. The reaction was added to ice water, then filtered and washed with H₂O and PE. The filter cake was triturated with a mixture of EA/PE (10 mL/200 mL) and filtered. The solid was co-evaporated with toluene twice to remove the water. This resulted in ethyl 5-(benzyloxy)-4-formyl-7-methoxy-2,3-dihydro-1H-indene-2-carboxylate (72.3 g, 160.32 mmol, 88% yield). The crude material was used without any further purification.

### Step 9: ethyl 5-(benzyloxy)-4-(1,3-dioxolan-2-yl)-7-methoxy-2,3-dihydro-1H-indene-2-carboxylate

Ethyl 5-(benzyloxy)-4-formyl-7-methoxy-2,3-dihydro-1H-indene-2-carboxylate (108 g, 304.739 mmol, 1 equiv.), ethylene glycol (94.6 g, 1524.135 mmol, 5.00 equiv.), 4-methylbenzene-1-sulfonate; pyridin-1-ium (7.7 g, 30.641 mmol, 0.10 equiv.), triethyl orthoformate (135.5 g, 914.293 mmol, 3.00 equiv.), a stir bar and toluene (1100 mL) were added to a 3000 mL flask. The resulting mixture was stirred at 90° C. for 2 h. After cooling down to r.t, the reaction was added to a mixture of saturated solution of NaHCO₃ and EA, then extracted with EA. The organic layers were dried over sodium sulphate, filtered and concentrated to dryness in vacuo. The residue obtained was triturated with the mixture of MTBE and Hexane (1/10) and filtered to afford ethyl 5-(benzyloxy)-4-(1,3-dioxolan-2-yl)-7-methoxy-2,3-dihydro-1H-indene-2-carboxylate (105 g, 254.47 mmol, 83% yield). The crude material was used without any further purification.

### Step 10: 5-(benzyloxy)-4-(1,3-dioxolan-2-yl)-7-methoxy-2,3-dihydro-1H-indene-2-carboxylic acid

Ethyl 5-(benzyloxy)-4-(1,3-dioxolan-2-yl)-7-methoxy-2,3-dihydro-1H-indene-2-carboxylate (105 g, 263.518 mmol, 1 equiv.), a stir bar, THF (900 mL) and H₂O (180 mL) were added to a 2000 mL flask, then treated with LiOH (9.47 g, 395.277 mmol, 1.5 equiv.). The resulting mixture was stirred at 70° C. for 2 h, then concentrated under reduced pressure. The residue obtained was triturated with MeCN and filtered to afford lithium 5-(benzyloxy)-4-(1,3-dioxolan-2-yl)-7-methoxy-2,3-dihydro-1H-indene-2-carboxylate (135 g, 253.25 mmol, 96% yield). The crude material was used without any further purification.

### Step 11: (1R,3S)-3-(3-(5-(benzyloxy)-4-formyl-7-methoxy-2,3-dihydro-1H-indene-2-carboxamido)-1H-pyrazol-5-yl)cyclopentyl 1,2,2-trimethylhydrazine-1-carboxylate

Lithium 5-(benzyloxy)-4-(1,3-dioxolan-2-yl)-7-methoxy-2,3-dihydro-1H-indene-2-carboxylate (120 g, 224.852 mmol, 1 equiv., 70.5%), (3-[[(ethylimino)methylidene]amino]propyl)dimethylamine hydrochloride (91.7 g, 478.352 mmol, 1.50 equiv.), a stir bar and pyridine (1200 mL) were added to a 3000 mL flask and stirred until homogenous, then treated with 1-{[(1R,3S)-3-(5-amino-2H-pyrazol-3-yl)cyclopentyl]oxy}-N,N′,N′-trimethylformohydrazide (85.2 g, 318.704 mmol, 1.00 equiv.). The resulting mixture was stirred at r.t for 1 h, then diluted with EA. The mixture was washed with H₂O and brine. The organic layers were dried over sodium sulphate, filtered, and concentrated to dryness in vacuo. To the crude residue a mixture of FA (500 mL) and H₂O (200 mL) were added and stirred at r.t overnight, then extracted with EA. The organic layers were washed with H₂O, NaHCO₃ solution, dried over sodium sulphate, filtered, and concentrated to dryness in vacuo. The residue was triturated with Et₂O and filtered to afford 5-(benzyloxy)-4-formyl-7-methoxy-N-{5-[(1S,3R)-3-[(N,N′,N′-trimethylhydrazinecarbonyl)oxy]cyclopentyl]-1H-pyrazol-3-yl}-2,3-dihydro-1H-indene-2-carboxamide (50 g, 78.22 mmol, 34% yield). The crude material was used without any further purification.

### Step 12: (1R,3S)-3-(5-((*)-4-formyl-5-hydroxy-7-methoxy-2,3-dihydro-H-indene-2-carboxamido)-1H-pyrazol-3-yl)cyclopentyl 1,2,2-trimethylhydrazine-1-carboxylate

5-(Benzyloxy)-4-formyl-7-methoxy-N-{5-[(1S,3R)-3-[(N,N′,N′-trimethylhydrazinecarbonyl)oxy]cyclopentyl]-1H-pyrazol-3-yl}-2,3-dihydro-1H-indene-2-carboxamide (32 g, 55.588 mmol, 1 equiv.), a stir bar, EA (300 mL) and tetrahydrofuran (300 mL) were added to a 1000 mL flask, then treated with Pd/C (16 g, 15.035 mmol, 0.27 equiv., 10%). The resulting mixture was maintained under H₂ and stirred at r.t for 3 h, then filtered and concentrated to dryness in vacuo. The residue obtained was purified by reverse phase chromatography, 5 to 35% MeCN/H₂O with 0.05% TFA modifier to afford a racemic product, which was further separated by chiral Prep-HPLC (Mobile Phase A: CO₂, Mobile Phase B: MeOH; Flow rate: 200 mL/min; Gradient (B %): isocratic 50% B; Back Pressure (bar): 100; Wave Length: 254 nm; RT1 (min): 5.05; RT2 (min): 7.02; Sample Solvent: MeOH; Injection Volume: 14.99 mL) to give two products. The first eluting was lyophilized to afford ((1R,3S)-3-(3-((*)-4-formyl-5-hydroxy-7-methoxy-2,3-dihydro-1H-indene-2-carboxamido)-1H-pyrazol-5-yl)cyclopentyl 1,2,2-trimethylhydrazine-1-carboxylate (Compound 284, 2.5173 g, 9.33% yield). 1H NMR (400 MHz, DMSO-d6) δ 12.10 (s, 1H), 11.24 (s, 1H), 10.42 (s, 1H), 10.07 (s, 1H), 6.54-6.22 (m, 2H), 5.06 (s, 1H), 3.84 (s, 3H), 3.51-3.41 (m, 2H), 3.30-3.09 (m, 2H), 3.03-2.91 (m, 1H), 2.90-2.75 (m, 4H), 2.57-2.50 (m, 6H), 2.45-2.31 (m, 1H), 2.14-1.99 (m, 1H), 1.94-1.64 (m, 4H), MS (ESI) calcd. for C₂₄H₃₁N₅O₆, 485.23 m/z, found: 486.20 [M+H]⁺.

### Example 39 NanoBRET Target Engagement (TE) Intracellular Kinase Assay

Inhibitory effects of compounds were measured in NanoBRET TE Intracellular Kinase Assay. 293T (ATCC; CRL-3216) or 293 (ATCC; CRL-1573) cell lines were maintained in 90% DMEM and 10% fetal bovine serum at 37° C., 5% CO₂, and 95% relative humidity. Cells were trypsinized and resuspended in assay media (99% Opti-MEM I Reduced Serum Medium, no phenol red, and 1% fetal bovine serum). Cells were co-transfected with 1 μg/mL of CDK-NanoLuc Fusion Vector (Promega; NV2701 for CDK1, NV2781 for CDK2, NV2811 for CDK4, NV2841 for CDK6) and 1 μg/mL of the corresponding Cyclin Expression Vector (Promega; NV2601 for CCNB1, NV2641 for CCNE1, NV2621 for CCND1, NV2631 for CCND3) using FuGENE HD Transfection Reagent (Promega; E2311). 40 uL/well of cells with transfection mixture were seeded in 384-well plates at 1.25×10⁵ to 2×10⁵ cells/mL and incubated for 24 hours at 37° C., 5% CO₂, and 95% relative humidity. On the next day, 100 nL of 100 μM NanoBRET Tracer K-10 (Promega; N2640) and 40 nL of 1000× test compounds prepared in DMSO were added to the cells in each well of 384-well plates for a 2-hour incubation at 37° C., 5% CO₂, and 95% relative humidity. 20 uL/well of 3× Intracellular TE Nano-Glo Substrate/Inhibitor (Promega; N2162) were added to 384-well plates and incubated for 3 minutes at room temperature. NanoBRET signals (donor emission wavelength at 450 nm, acceptor emission wavelength at 610 nm) were measured using Envision plate reader (PerkinElmer). NanoBRET ratio was calculated by: BRET Ratio=raw signal for acceptor/raw signal for donor. Dose response curve normalized to DMSO control was plotted using four-parameter logistic model for IC₅₀ calculation.

### Example 40 OVCAR3 Phospho-Rb Ser807/811 HTRF Assay

Inhibitory effects of compounds were measured in OVCAR3 Phospho-Rb Ser807/811 HTRF assay. OVCAR3 cell line (ATCC; HTB-161) was maintained in 90% RPMI and 10% fetal bovine serum at 37° C., 5% C02, and 95% relative humidity. Cells were trypsinized and resuspended in 1 mM hydroxyurea (Sigma; H8627), 90% RPMI and 10% fetal bovine serum. 100 μL of cells were seeded at 40,000 to 90,000 cells/well in 96-well plates and incubated for 24 hours at 37° C., 5% CO₂, and 95% relative humidity. On the next day, 250 nL of test compounds prepared in DMSO were added to the cells in each well of 96-well plates and incubated for 1 hour at 37° C., 5% CO₂, and 95% relative humidity. Cells were lysed with 50 μL of 1× lysis buffer from Phospho-Rb (Ser807/811) Cellular Kit (Cisbio; 64RBS807PEG) with shaking at room temperature for 45 minutes. 16 μL of cell lysates were transferred to ProxiPlate-384 Plus, White 384-shallow well Microplate (PerkinElmer; 6008289). 2 μL of Phospho-Rb Eu Cryptate antibody and 2 μL of Phospho-Rb d2 antibody prepared in the detection buffer from Phospho-Rb (Ser807/811) Cellular Kit (Cisbio; 64RBS807PEG) were added to each well containing cell lysates and incubated for 2.5 to 24 hours at room temperature. HTRF signals (fluorescent emission at two different wavelengths at 665 nm and 620 nm) were measured using Envision plate reader (PerkinElmer) or PHERAstar FSX plate reader (BMG LABTECH). HTRF ratio was calculated by: HTRF ratio=raw signal at 665 nm/raw signal at 620 nm. Dose response curve normalized to DMSO control was plotted using four-parameter logistic model for IC₅₀ calculation.

Bioassay results for selected compounds according to Examples 21-38 are provided in Table 2.

TABLE 2
Bioassay Data For Selected Compounds.

| | NanoBRET- | NanoBRET- | NanoBRET- | |
|---|---|---|---|---|
| Compound | CDK1/B1 IC₅₀ | CDK2/E1 IC50 | CDK4/D1 IC50 | pRb-OVCAR3 |
| Number | (nM) | (nM) | (nM) | IC50 (nM) |
| 1 | 62.9 | 7.87 | 1800 | 274 |
| 2 | 3.45 | 3.82 | 89.5 | 68 |
| 3 | 32.8 | 9.48 | 255 | 129 |
| 4 | 7.08 | 5.47 | 6.91 | 68.8 |
| 5 | 5.54 | 6.58 | 52.6 | 187 |
| 6 | 113 | 12.5 | 397 | 618 |
| 7 | 275 | 11.4 | 1850 | 1500 |
| 8 | 73.5 | 24.9 | 5460 | 880 |
| 9 | 50.3 | 29.2 | 4700 | 449 |
| 10 | 11.3 | 6.04 | 1000 | 67.6 |
| 11 | 17.3 | 3.72 | >9.90E+03 | 102 |
| 63 | 361 | 9.55 | 6320 | 1260 |
| 12 | 126 | 11.1 | 1450 | — |
| 13 | 92.9 | 12.4 | 4700 | 701 |
| 14 | 1690 | 138 | 779 | — |
| 15 | 25.3 | 2.65 | 1820 | 112 |
| 16 | 75.9 | 34.3 | 626 | 585 |
| 17 | 16.7 | 4.87 | 4580 | 226 |
| 18 | 703 | 11.4 | 1610 | 4640 |
| 19 | 78 | 16.4 | 2420 | 941 |
| 20 | 147 | 10.5 | 1210 | 915 |
| 21 | 290 | 5.53 | 3900 | 2120 |
| 22 | 100 | 4.81 | 3690 | 200 |
| 23 | 125 | 10.9 | 969 | — |
| 24 | 516 | 18.7 | 4880 | 3760 |
| 25 | 299 | 9.94 | 1210 | 1330 |
| 26 | 21.7 | 2.78 | 75.1 | 94.5 |
| 27 | 17.7 | 5.24 | 112 | 95.2 |
| 28 | 151 | 10.4 | 1190 | 1820 |
| 29 | 494 | 18.6 | 1550 | 2150 |
| 30 | 491 | 17.8 | 3160 | 2740 |
| 31 | 20.4 | 5.46 | 3070 | 214 |
| 32 | 52 | 4.66 | 4920 | 352 |
| 33 | 71.6 | 11.3 | 361 | 361 |
| 34 | 98.9 | 5.34 | 196 | 552 |
| 35 | 537 | 23.9 | 3960 | 4130 |
| 36 | 82.2 | 8.17 | 1910 | 702 |
| 37 | 176 | 15.6 | 128 | 344 |
| 38 | 264 | 10.6 | 576 | 542 |
| 39 | 351 | 24.7 | 474 | 1130 |
| 40 | 275 | 24.4 | 1460 | 796 |
| 41 | 20.5 | 5.32 | 3060 | 272 |
| 42 | 10.6 | 5.5 | 2680 | 176 |
| 43 | 51.2 | 27.2 | >10.0E+03 | 212 |
| 44 | 168 | 6.37 | 1550 | 485 |
| 45 | 6.81 | 2.8 | 1580 | 114 |
| 46 | 692 | 20.6 | 4170 | 2410 |
| 47 | 224 | 98.9 | 9430 | 1170 |
| 48 | 10.8 | 2.28 | 848 | 125 |
| 49 | 70.2 | 24.5 | >10.0E+03 | 161 |
| 50 | 49.7 | 8.63 | >10.0E+03 | 382 |
| 51 | 354 | 7.58 | 4100 | 1150 |
| 52 | 383 | 21.7 | 543 | 2430 |
| 53 | 21.7 | 9.39 | 3660 | 361 |
| 54 | 175 | 23.4 | >10.0E+03 | 1690 |
| 55 | 69.9 | 21.9 | >10.0E+03 | 1430 |
| 56 | 4.83 | 1.32 | >10.0E+03 | 75.8 |
| 57 | 21.9 | 3.06 | >9.58E+03 | 110 |
| 58 | 64.3 | 9.38 | 7090 | 1190 |
| 59 | 362 | 32.1 | >10.0E+03 | 2300 |
| 60 | 15.1 | 6.5 | 3630 | 230 |
| 61 | 36.1 | 6.27 | 2890 | 477 |
| 62 | 270 | 34.4 | 7230 | 1870 |
| 64 | 9.52 | 2.76 | >6.43E+03 | 33.2 |
| 65 | 28.1 | 8.52 | 4630 | 318 |
| 66 | 390 | 8.87 | 779 | 1690 |
| 67 | 35.3 | 6.73 | 202 | 475 |
| 68 | 39.2 | 5.56 | 1150 | 662 |
| 69 | 79 | 1.92 | 1950 | 730 |
| 70 | 337 | 10.1 | 699 | 2680 |
| 71 | 10.3 | 7.8 | 4110 | 85.5 |
| 72 | 436 | 8.38 | >10.0E+03 | 477 |
| 73 | 61 | 26.7 | >10.0E+03 | 299 |
| 74 | 29.5 | 7.06 | 3950 | 523 |
| 75 | 372 | 21.4 | 224 | 4970 |
| 76 | 66 | 5.01 | 6390 | 309 |
| 77 | 8.93 | 3.05 | 44.3 | 46.5 |
| 78 | 142 | 16.7 | > 10.0E+03 | 1190 |
| 79 | 47 | 14.2 | 727 | 505 |
| 80 | 37.3 | 25.4 | >10.0E+03 | 132 |
| 81 | 18.8 | 2.3 | >10.0E+03 | 119 |
| 82 | 11.3 | 3.32 | 4420 | 43.2 |
| 83 | 6.23 | 4.75 | 2490 | 83.3 |
| 84 | 15.1 | 5.93 | 5200 | 228 |
| 85 | 23.7 | 2.6 | >10.0E+03 | 177 |
| 86 | 32.5 | 3.73 | >10.0E+03 | 212 |
| 87 | 24.3 | 2.77 | 9800 | 208 |
| 88 | 42 | 3.47 | >10.0E+03 | 227 |
| 89 | 11.3 | 2.27 | 1280 | 44.7 |
| 90 | 213 | 43.8 | 5100 | 761 |
| 91 | 37.3 | 11.6 | 3270 | 877 |
| 92 | 278 | 16.3 | 3840 | 2400 |
| 93 | 169 | 8.52 | 2280 | 391 |
| 94 | 165 | 9.73 | 1310 | 1440 |
| 95 | 21.5 | 1.26 | >10.0E+03 | 115 |
| 96 | 129 | 7.13 | 1820 | 601 |
| 97 | 1140 | 8.75 | 9440 | 3020 |
| 98 | 1490 | 12 | >10.0E+03 | 3340 |
| 99 | 21.4 | 3.14 | >10.0E+03 | 259 |
| 100 | 12.8 | 7.32 | 8710 | 120 |
| 101 | 20.3 | 5.79 | 9200 | 188 |
| 102 | 8.39 | 4.16 | 2860 | 78.2 |
| 103 | 5.66 | 1.43 | 3790 | 31.7 |
| 105 | 2.18 | 1.01 | 2350 | 65.8 |
| 106 | 15.4 | 4.3 | 3330 | 306 |
| 107 | 12 | 4.4 | 224 | 59.6 |
| 108 | 15.2 | 3.65 | >10.0E+03 | 44.4 |
| 109 | 4.14 | 1.85 | 4370 | 17.6 |
| 110 | 7.64 | 2.9 | 5360 | 27.7 |
| 111 | 21 | 5.45 | >10.0E+03 | 79.8 |
| 112 | 36 | 4.69 | > 10.0E+03 | 149 |
| 113 | 11.4 | 4.13 | >10.0E+03 | 61.6 |
| 114 | 26.7 | 4.44 | >10.0E+03 | 121 |
| 115 | | 1.94 | 1240 | 16.3 |
| 116 | | 4.5 | 4360 | 32.3 |
| 117 | | 2.19 | 8090 | 22 |
| 118 | | 55.4 | 6480 | 101 |
| 119 | | 2.31 | 7860 | 26.4 |
| 120 | | 2.64 | 2970 | 13.4 |
| 121 | | 1.99 | 2440 | 18.5 |
| 122 | | 1.99 | 1310 | 20.9 |
| 123 | | 2.62 | 2570 | 27 |
| 124 | | 1.99 | 2580 | 37.7 |
| 125 | | 3.12 | 1540 | 53.4 |
| 126 | | 9.62 | 7310 | 75.7 |
| 127 | | 25.9 | 8530 | 98.3 |
| 128 | 5.09 | 2.42 | 961 | 34.8 |
| 129 | | 1.96 | 7620 | 44.5 |
| 130 | | 3.69 | 864 | 24.9 |
| 131 | | 3.25 | 697 | 12.5 |
| 132 | 10.8 | 3.39 | 2940 | 22 |
| 133 | | 4.42 | 2800 | 19.6 |
| 134 | | 3.26 | 3800 | 37.1 |
| 135 | | 3.41 | >10.0E+03 | 92.7 |
| 136 | | 2.22 | 1420 | 16.9 |
| 137 | | 21.6 | >10.0E+03 | 295 |
| 138 | | 4.71 | 5860 | 80.6 |
| 139 | | 12.9 | 3990 | 178 |
| 140 | | 2.82 | 1080 | 16.1 |
| 141 | | 1.87 | 5500 | 100 |
| 142 | | 3.76 | 7020 | 53.3 |
| 143 | | 13.1 | >10.0E+03 | 142 |
| 144 | | 2.36 | 2340 | 29 |
| 145 | | 3.46 | 4070 | 64.3 |
| 146 | | 9.88 | 2380 | 40.9 |
| 147 | | 2.85 | 2260 | 13.8 |
| 148 | | 6.97 | 4250 | 113 |
| 149 | | 20.2 | >10.0E+03 | 385 |
| 150 | | 2.27 | 3890 | 148 |
| 151 | | 3.47 | 2890 | 36.1 |
| 152 | | 7.62 | 6990 | 30 |
| 153 | | 13.1 | >10.0E+03 | 49.7 |
| 154 | | 7.73 | 2100 | 27.8 |
| 155 | | 7.65 | 6300 | 23.5 |
| 156 | | 10.1 | >10.0E+03 | 33.8 |
| 157 | | 6.79 | 7000 | 70 |
| 158 | | 5.58 | 3900 | 24.4 |
| 159 | | 10.6 | 4840 | 43.1 |
| 160 | | 7.35 | >10.0E+03 | 80.3 |
| 161 | | 2.79 | 7360 | 68.6 |
| 162 | | 3.09 | 3470 | 37.4 |
| 163 | | 10.2 | 6300 | 27.7 |
| 164 | | 9.6 | 5880 | 301 |
| 165 | | 12.8 | 7920 | 383 |
| 166 | | 10.3 | >10.0E+03 | 167 |
| 167 | | 2.8 | 828 | 24.3 |
| 168 | | 2.45 | 3270 | 29.2 |
| 169 | | 2.27 | 773 | 22.9 |
| 170 | | 3.06 | 6040 | 70.6 |
| 171 | | 3.24 | >10.0E+03 | 107 |
| 172 | 25.2 | 3.95 | >10.0E+03 | 132 |
| 173 | | 3.99 | 5340 | 34.2 |
| 174 | | 3.26 | 668 | 10.7 |
| 175 | | 2.11 | 8440 | 16.9 |
| 176 | | 4.2 | 3360 | 57.4 |
| 177 | | 3.17 | > 10.0E+03 | 29.3 |
| 178 | | 2.08 | 5600 | 54.1 |
| 179 | | 14.1 | >10.0E+03 | 115 |
| 180 | | 2.68 | 1580 | 23.5 |
| 181 | | 1.18 | 5000 | 27.9 |
| 182 | | 6.24 | >10.0E+03 | 66.1 |
| 183 | | 10.1 | 4200 | 140 |
| 184 | | 1.5 | 1450 | 20.6 |
| 185 | | 445 | >10.0E+03 | 363 |
| 186 | | 22 | 6580 | 105 |
| 187 | | 18.4 | >10.0E+03 | 65.3 |
| 188 | | 337 | >10.0E+03 | 368 |
| 189 | | 159 | >10.0E+03 | 198 |
| 190 | | 8.02 | 4160 | 35 |
| 191 | | 25 | >10.0E+03 | 127 |
| 192 | | 24 | 5910 | 88 |
| 193 | | 11.6 | >10.0E+03 | 84.7 |
| 194 | | 17.1 | 2890 | 295 |
| 195 | | 9.37 | 5000 | 37.8 |
| 196 | | 6.2 | 7360 | 102 |
| 197 | | 7.53 | >10.0E+03 | 94.7 |
| 198 | 7 | 1.92 | 3220 | 38.7 |
| 199 | 19.9 | 3.76 | 6500 | 58.5 |
| 200 | 8.83 | 5.07 | 8410 | 27.7 |
| 201 | 12.9 | 4.1 | 4680 | 28.4 |
| 202 | 41.6 | 5.67 | > 10.0E+03 | 70.5 |
| 203 | 18.6 | 7.37 | >10.0E+03 | 60 |
| 204 | 12.2 | 4.54 | >9.51E+03 | 34.6 |
| 205 | 4.59 | 3.48 | 2040 | 26.3 |
| 206 | 5.62 | 2.46 | >10.0E+03 | 27.4 |
| 207 | 6.54 | 2.25 | 4130 | 25.7 |
| 208 | 21.2 | 9.87 | 7910 | 41.8 |
| 209 | 5.94 | 6.55 | 3870 | 28.7 |
| 210 | 26.3 | 6.9 | >10.0E+03 | 68.5 |
| 211 | 9.01 | 2.68 | 2120 | 34.5 |
| 212 | 6.31 | 2.98 | 1070 | 21.8 |
| 213 | 30.1 | 4.72 | >10.0E+03 | 35.6 |
| 214 | 155 | 56.8 | > 3.46E+03 | 375 |
| 215 | 20.6 | 10.3 | >6.27E+03 | 41.5 |
| 216 | 15 | 4.44 | 9910 | 64.6 |
| 217 | 44.7 | 2.98 | >10.0E+03 | 60.9 |
| 218 | 5.53 | 2.33 | 2900 | 18.2 |
| 219 | 4.23 | 1.95 | 844 | 22.6 |
| 220 | 8.36 | 2.89 | 1460 | 32.1 |
| 221 | 8.56 | 1.71 | 5250 | 24.1 |
| 222 | 7.61 | 3.84 | 2830 | 63.7 |
| 223 | 13.1 | 4.13 | >10.0E+03 | 65.6 |
| 224 | 28.9 | 2.64 | >10.0E+03 | 93.6 |
| 225 | 5.09 | 2.49 | 2600 | 18.7 |
| 226 | 9.17 | 2.45 | 3060 | 63.7 |
| 227 | 0.61 | 1.73 | 1290 | 18.1 |
| 228 | 4.25 | 2.27 | 4170 | 34.4 |
| 229 | 1.97 | 1.75 | 1900 | 28.8 |
| 230 | 21 | 2.85 | >10.0E+03 | 57.8 |
| 231 | <2.64 | 3.2 | 1430 | 18.5 |
| 232 | 4.84 | 2.18 | 2300 | 40.2 |
| 233 | 2.19 | 2.47 | 2690 | 22.1 |
| 234 | | 1.18 | 2840 | 14.2 |
| 235 | 48.5 | 7.6 | >10.0E+03 | 121 |
| 236 | 12.8 | 1.9 | 4250 | 32.2 |
| 237 | 11.6 | 1.79 | 4960 | 55.7 |
| 238 | 9.82 | 1.64 | 4100 | 32.8 |
| 239 | 2.14 | 1.89 | 846 | 13.1 |
| 240 | 7.4 | 1.54 | 2800 | 22.1 |
| 241 | 22.9 | 3.05 | 3850 | 77.6 |
| 242 | 13.1 | 5.05 | >10.0E+03 | 66.9 |
| 243 | 2.41 | 2.23 | 484 | 17.6 |
| 244 | 4.17 | 1.42 | 1320 | 21.2 |
| 245 | 3.95 | 3.12 | 723 | 25.4 |
| 246 | 9.45 | 2.78 | 6110 | 33 |
| 247 | 19.4 | 15.8 | 7210 | 101 |
| 248 | 106 | 20.2 | 575 | 1950 |
| 249 | 4.5 | 1.5 | 1190 | 26.6 |
| 250 | 37.3 | 1.86 | 9240 | 65.8 |
| 251 | 4.13 | 1.22 | 966 | 15.1 |
| 252 | 30.6 | 6.48 | 4090 | 326 |
| 253 | 18.4 | 2.65 | 5100 | 28.7 |
| 254 | 2.8 | 2.28 | 778 | 21.4 |
| 255 | 10.9 | 2.24 | 4270 | 32.8 |
| 256 | 20.3 | 3.76 | 4110 | 68.9 |
| 257 | 703 | 10.5 | >10.0E+03 | 2490 |
| 258 | 269 | 5.38 | >10.0E+03 | 556 |
| 259 | 8060 | 649 | >10.0E+03 | |
| 260 | | 15 | >10.0E+03 | 170 |
| 261 | | 4.7 | 4100 | 51 |
| 262 | | 4.42 | 5080 | 51.8 |
| 263 | | 63.3 | 735 | 7740 |
| 264 | | 5.95 | 512 | 13.6 |
| 265 | | 15.5 | 132 | 567 |
| 266 | | 1.99 | 1980 | 123 |
| 267 | | 21.8 | 266 | 5230 |
| 268 | | 10.8 | 4940 | 85.9 |
| 269 | | 67.7 | 459 | 2230 |
| 270 | | 6.25 | >10.0E+03 | 48.6 |
| 271 | | 240 | 3250 | >10.0E+03 |
| 272 | | 2.2 | >10.0E+03 | 39.3 |
| 273 | | 2.38 | 5820 | 29.5 |
| 274 | | 3.17 | >10.0E+03 | 54.7 |
| 275 | | 3.11 | 5300 | 35.7 |
| 276 | | 5.09 | >10.0E+03 | 31.6 |
| 277 | | 1.48 | 5090 | 25.6 |
| 278 | | 4.1 | >10.0E+03 | 95.9 |
| 279 | | 4.32 | >10.0E+03 | 63.9 |
| 280 | | 3.88 | 576 | 17.4 |
| 281 | | 4.85 | 1200 | 27.3 |
| 282 | | 5.3 | 3350 | 20.4 |
| 283 | | 2.01 | 236 | 12.6 |
| 284 | | 1.85 | 2850 | 29.2 |

### Example 41 Intact Mass Spectrometry and Competition Studies

### Identification of Covalent Binding by Intact Mass Spectrometry for Compounds

pCDK2-CCNE1 proteins (1 μM) were treated with a compound (1.5 μM) from Table 4 for 1 hour at room temperature or 37° C., with and without competitor molecule PF-06873600 (20 μM final concentration) as a specificity control. As used herein, the competitor molecule is

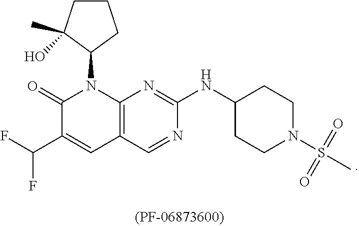 [SMILES: C[C@@]1(O)CCC[C@H]1n1c(=O)c(C(F)F)cc2cnc(NC3CCN(S(C)(=O)=O)CC3)nc21]

As used herein, the competitor molecule is 6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one (PF-06873600). Reversible imines were captured by reductive amination with the addition of 5 mM sodium borohydride at room temperature for 5 min. Reduction reactions were quenched by diluting the sample in equal volume of 1% trifluoroacetic acid (v/v) in acetonitrile, then analyzed by LC-MS. Deconvoluted monoisotopic intensities of unmodified and compound-modified mass peaks were compared.

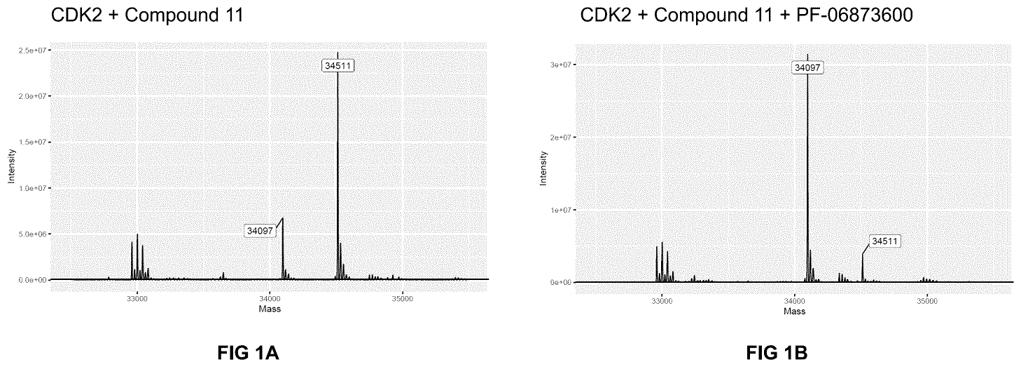

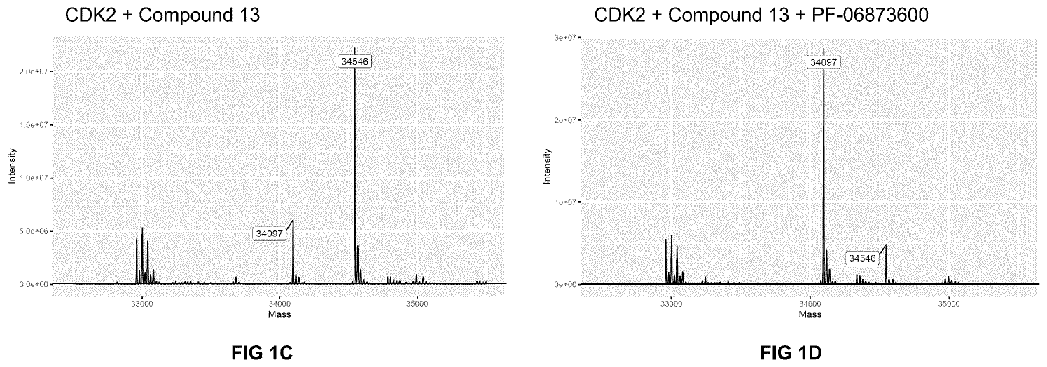

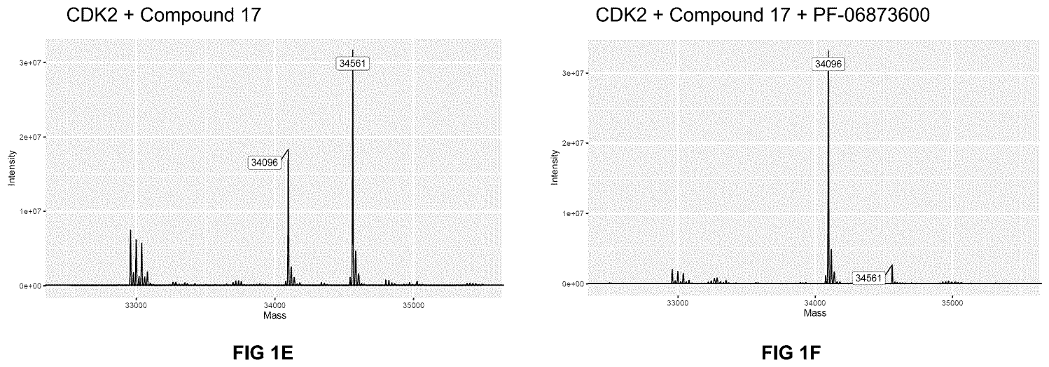

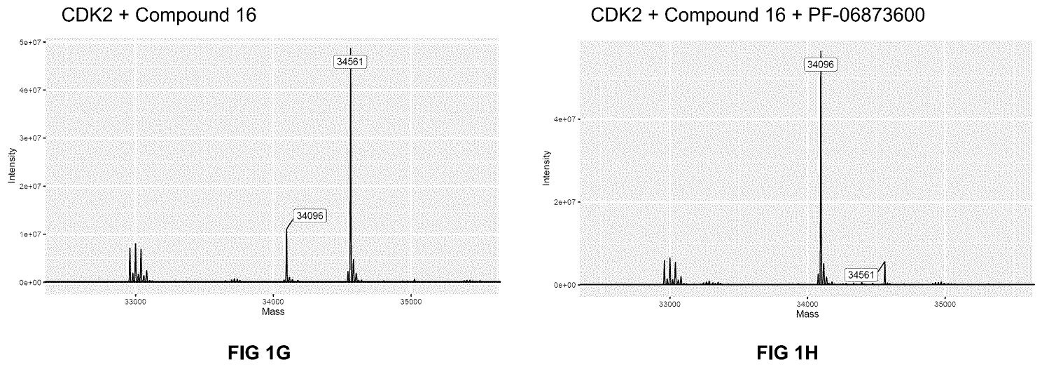

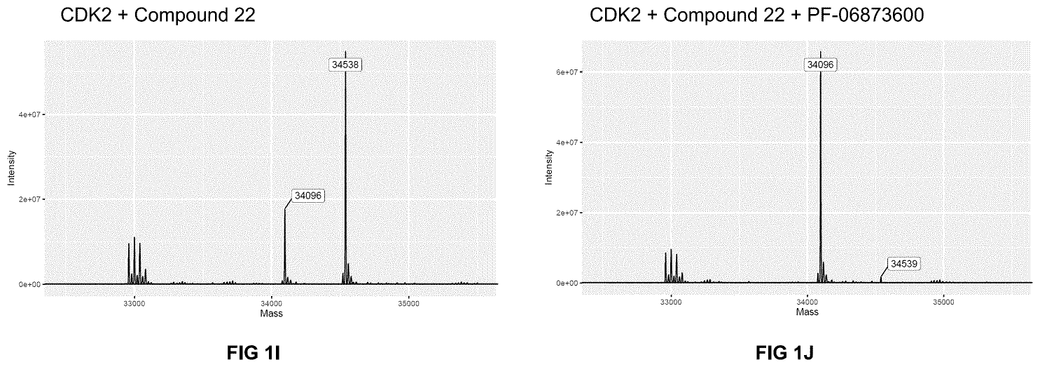

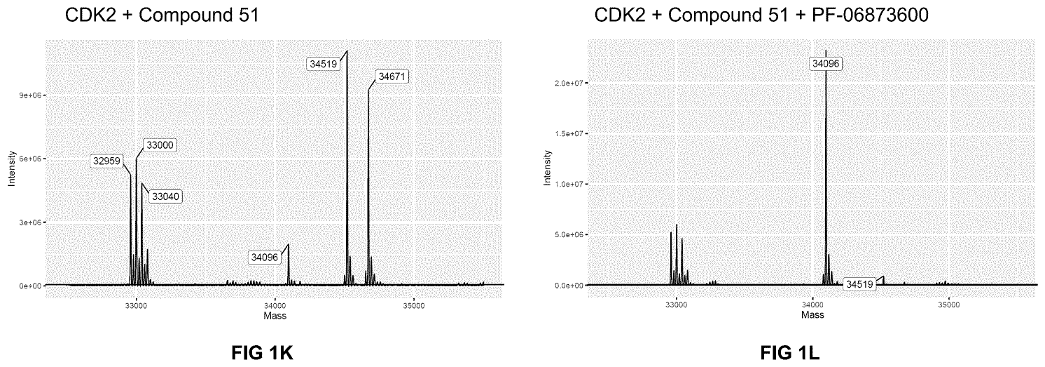

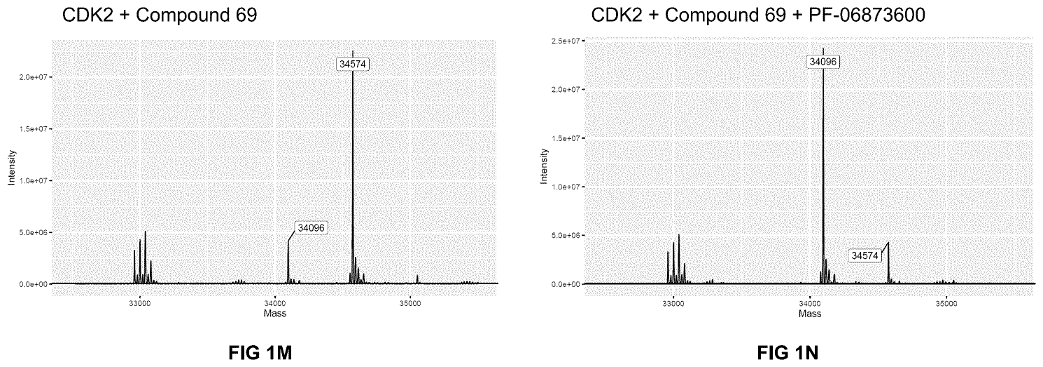

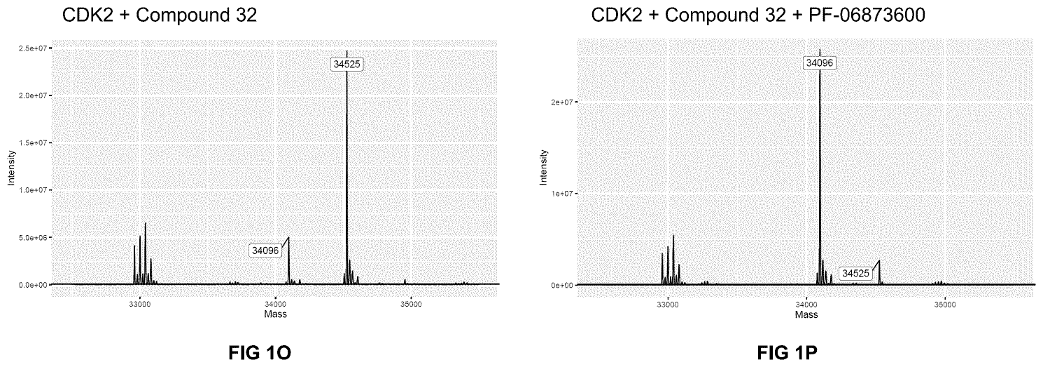

FIGS. 1A to 1P provide intact mass spectra for pCDK2-CCNE1 proteins after incubation with compounds of the present disclosure and subsequent sodium borohydride reduction. Sodium borohydride was added to reduce the reversible imine bond between the pCDK2-CCNE1 proteins and compounds of the present disclosure, thereby forming a more stable C—N single bond between the protein and the compound and allowing detection of a covalent complex by intact mass spectrometry. Further, FIGS. 1A to 1P each provide intact mass spectra following the incubation of pCDK2-CCNE1 proteins with a compound of the present disclosure both in the absence of a competitor molecule (i.e., PF-06873600, a molecule known to bind to and inhibit the function of CDK2); and in the presence of a significant stoichiometric excess of a competitor molecule PF-06873600.

FIG. 1A provides intact mass spectra following incubation of Compound 11 with pCDK2-CCNE1 proteins both in the absence of any competitor molecule (FIG. 1A) and in the presence of a significant molar excess of competitor molecule PF-06873600 (FIG. 1B). Incubation of Compound 11 with pCDK2-CCNE1 proteins resulted in covalent complex formation between Compound 11 and the CDK2 protein. The data provided demonstrates two different m/z peaks, which correspond to the mass of the unmodified CDK2 protein and the covalent complex between Compound 11 and CDK2 protein. The peak with specific observed mass value of 34096-34097 Da corresponds to the unmodified CDK2 protein. The second peak to the right of the first with specific observed mass value of 34511 Da corresponds to the covalent complex formed between Compound 11 and CDK2 protein. Thus, as seen in FIG. 1A a noticeable level of covalent complex was formed between Compound 11 and CDK2 protein, indicated by relative intensity of the peak corresponding to the covalent complex compared to those corresponding to the unmodified protein. Furthermore, the extent of covalent complex formation was likely to be even higher than shown in FIG. 1A, as some of the peak intensity for the peak corresponding to unmodified protein was attributable to fragmentation of the C—N bond between Compound 11 and the CDK2 protein (formed by borohydride reduction of the imine bond in the covalent complex) during ionization and detection. In contrast, FIG. 1B demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1A and 1B show that (1) Compound 11 formed a covalent complex with CDK2 protein, and (2) Compound 11 bound to the same binding pocket of CDK2 as the competitor molecule PF-06873600, as shown by the significant reduction in observed complex formation in the presence of a stoichiometric excess of competitor molecule (compare FIGS. 1A to 1B).

FIG. 1C provides intact mass spectra following incubation of Compound 13 with pCDK2-CCNE1 proteins both in the absence of any competitor molecule (FIG. 1C) and in the presence of a significant molar excess of competitor molecule PF-06873600 (FIG. 1D). Incubation of Compound 13 with pCDK2-CCNE1 proteins resulted in covalent complex formation between Compound 13 and the CDK2 protein. The data provided demonstrates two different m/z peaks, which correspond to the mass of the unmodified CDK2 protein and the covalent complex between Compound 13 and CDK2 protein. The peak with specific observed mass value of 34096-34097 Da corresponds to the unmodified CDK2 protein. The second peak to the right of the first with specific observed mass value of 34546 Da corresponds to the covalent complex formed between Compound 13 and CDK2 protein. Thus, as seen in FIG. 1C a noticeable level of covalent complex was formed between Compound 13 and CDK2 protein, indicated by relative intensity of the peak corresponding to the covalent complex compared to those corresponding to the unmodified protein. Furthermore, the extent of covalent complex formation was likely to be even higher than shown in FIG. 1C, as some of the peak intensity for the peak corresponding to unmodified protein was attributable to fragmentation of the C—N bond between Compound 13 and the CDK2 protein (formed by borohydride reduction of the imine bond in the covalent complex) during ionization and detection. In contrast, FIG. 1D demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1C and 1D show that (1) Compound 13 formed a covalent complex with CDK2 protein, and (2) Compound 13 bound to the same binding pocket of CDK2 as the competitor molecule PF-06873600, as shown by the significant reduction in observed complex formation in the presence of a stoichiometric excess of competitor molecule (compare FIGS. 1C to 1D).

FIG. 1E provides intact mass spectra following incubation of Compound 17 with pCDK2-CCNE1 proteins both in the absence of any competitor molecule (FIG. 1E) and in the presence of a significant molar excess of competitor molecule PF-06873600 (FIG. 1F). Incubation of Compound 17 with pCDK2-CCNE1 proteins resulted in covalent complex formation between Compound 17 and the CDK2 protein. The data provided demonstrates two different m/z peaks, which correspond to the mass of the unmodified CDK2 protein and the covalent complex between Compound 17 and CDK2 protein. The peak with specific observed mass value of 34096-34097 Da corresponds to the unmodified CDK2 protein. The second peak to the right of the first with specific observed mass value of 34561 Da corresponds to the covalent complex formed between Compound 17 and CDK2 protein. Thus, as seen in FIG. 1E a noticeable level of covalent complex was formed between Compound 17 and CDK2 protein, indicated by relative intensity of the peak corresponding to the covalent complex compared to those corresponding to the unmodified protein. Furthermore, the extent of covalent complex formation was likely to be even higher than shown in FIG. 1E, as some of the peak intensity for the peak corresponding to unmodified protein was attributable to fragmentation of the C—N bond between Compound 17 and the CDK2 protein (formed by borohydride reduction of the imine bond in the covalent complex) during ionization and detection. In contrast, FIG. 1F demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1E and 1F show that (1) Compound 17 formed a covalent complex with CDK2 protein, and (2) Compound 17 bound to the same binding pocket of CDK2 as the competitor molecule PF-06873600, as shown by the significant reduction in observed complex formation in the presence of a stoichiometric excess of competitor molecule (compare FIGS. 1E to 1F).

FIG. 1G provides intact mass spectra following incubation of Compound 16 with pCDK2-CCNE1 proteins both in the absence of any competitor molecule (FIG. 1G) and in the presence of a significant molar excess of competitor molecule PF-06873600 (FIG. 1H). Incubation of Compound 16 with pCDK2-CCNE1 proteins resulted in covalent complex formation between Compound 16 and the CDK2 protein. The data provided demonstrates two different m/z peaks, which correspond to the mass of the unmodified CDK2 protein and the covalent complex between Compound 16 and CDK2 protein. The peak with specific observed mass value of 34096-34097 Da corresponds to the unmodified CDK2 protein. The second peak to the right of the first with specific observed mass value of 34561 Da corresponds to the covalent complex formed between Compound 16 and CDK2 protein. Thus, as seen in FIG. 1G a noticeable level of covalent complex was formed between Compound 16 and CDK2 protein, indicated by relative intensity of the peak corresponding to the covalent complex compared to those corresponding to the unmodified protein. Furthermore, the extent of covalent complex formation was likely to be even higher than shown in FIG. 1G, as some of the peak intensity for the peak corresponding to unmodified protein was attributable to fragmentation of the C—N bond between Compound 16 and the CDK2 protein (formed by borohydride reduction of the imine bond in the covalent complex) during ionization and detection. In contrast, FIG. 1H demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1G and 1H show that (1) Compound 16 formed a covalent complex with CDK2 protein, and (2) Compound 16 bound to the same binding pocket of CDK2 as the competitor molecule PF-06873600, as shown by the significant reduction in observed complex formation in the presence of a stoichiometric excess of competitor molecule (compare FIGS. 1G to 1H).

FIG. 1I provides intact mass spectra following incubation of Compound 22 with pCDK2-CCNE1 proteins both in the absence of any competitor molecule (FIG. 1I) and in the presence of a significant molar excess of competitor molecule PF-06873600 (FIG. 1J). Incubation of Compound 22 with pCDK2-CCNE1 proteins resulted in covalent complex formation between Compound 22 and the CDK2 protein. The data provided demonstrates two different m/z peaks, which correspond to the mass of the unmodified CDK2 protein and the covalent complex between Compound 22 and CDK2 protein. The peak with specific observed mass value of 34096-34097 Da corresponds to the unmodified CDK2 protein. The second peak to the right of the first with specific observed mass value of 34538-34539 Da corresponds to the covalent complex formed between Compound 22 and CDK2 protein. Thus, as seen in FIG. 1I a noticeable level of covalent complex was formed between Compound 22 and CDK2 protein, indicated by relative intensity of the peak corresponding to the covalent complex compared to those corresponding to the unmodified protein. Furthermore, the extent of covalent complex formation was likely to be even higher than shown in FIG. 1I, as some of the peak intensity for the peak corresponding to unmodified protein was attributable to fragmentation of the C—N bond between Compound 22 and the CDK2 protein (formed by borohydride reduction of the imine bond in the covalent complex) during ionization and detection. In contrast, FIG. 1J demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1I and 1J show that (1) Compound 22 formed a covalent complex with CDK2 protein, and (2) Compound 22 bound to the same binding pocket of CDK2 as the competitor molecule PF-06873600, as shown by the significant reduction in observed complex formation in the presence of a stoichiometric excess of competitor molecule (compare FIGS. 1I to 1J).

FIG. 1K provides intact mass spectra following incubation of Compound 51 with pCDK2-CCNE1 proteins both in the absence of any competitor molecule (FIG. 1K) and in the presence of a significant molar excess of competitor molecule PF-06873600 (FIG. 1L). Incubation of Compound 51 with pCDK2-CCNE1 proteins resulted in covalent complex formation between Compound 51 and the CDK2 protein. The data provided demonstrates two different m/z peaks, which correspond to the mass of the unmodified CDK2 protein and the covalent complex between Compound 51 and CDK2 protein. The peak with specific observed mass value of 34096-34097 Da corresponds to the unmodified CDK2 protein. The second peak to the right of the first with specific observed mass value of 34519 Da corresponds to the covalent complex formed between Compound 51 and CDK2 protein. Thus, as seen in FIG. 1K a noticeable level of covalent complex was formed between Compound 51 and CDK2 protein, indicated by relative intensity of the peak corresponding to the covalent complex compared to those corresponding to the unmodified protein. Furthermore, the extent of covalent complex formation was likely to be even higher than shown in FIG. 1K, as some of the peak intensity for the peak corresponding to unmodified protein was attributable to fragmentation of the C—N bond between Compound 51 and the CDK2 protein (formed by borohydride reduction of the imine bond in the covalent complex) during ionization and detection. In contrast, FIG. 1L demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1K and 1L show that (1) Compound 51 formed a covalent complex with CDK2 protein, and (2) Compound 51 bound to the same binding pocket of CDK2 as the competitor molecule PF-06873600, as shown by the significant reduction in observed complex formation in the presence of a stoichiometric excess of competitor molecule (compare FIGS. 1K to 1L).

FIG. 1M provides intact mass spectra following incubation of Compound 69 with pCDK2-CCNE1 proteins both in the absence of any competitor molecule (FIG. 1M) and in the presence of a significant molar excess of competitor molecule PF-06873600 (FIG. 1N). Incubation of Compound 69 with pCDK2-CCNE1 proteins resulted in covalent complex formation between Compound 69 and the CDK2 protein. The data provided demonstrates two different m/z peaks, which correspond to the mass of the unmodified CDK2 protein and the covalent complex between Compound 69 and CDK2 protein. The peak with specific observed mass value of 34096-34097 Da corresponds to the unmodified CDK2 protein. The second peak to the right of the first with specific observed mass value of 34574 Da corresponds to the covalent complex formed between Compound 69 and CDK2 protein. Thus, as seen in FIG. 1M a noticeable level of covalent complex was formed between Compound 69 and CDK2 protein, indicated by relative intensity of the peak corresponding to the covalent complex compared to those corresponding to the unmodified protein. Furthermore, the extent of covalent complex formation was likely to be even higher than shown in FIG. 1M, as some of the peak intensity for the peak corresponding to unmodified protein was attributable to fragmentation of the C—N bond between Compound 69 and the CDK2 protein (formed by borohydride reduction of the imine bond in the covalent complex) during ionization and detection. In contrast, FIG. 1N demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1M and 1N show that (1) Compound 69 formed a covalent complex with CDK2 protein, and (2) Compound 69 bound to the same binding pocket of CDK2 as the competitor molecule PF-06873600, as shown by the significant reduction in observed complex formation in the presence of a stoichiometric excess of competitor molecule (compare FIGS. 1M to 1N).

FIG. 1O provides intact mass spectra following incubation of Compound 32 with pCDK2-CCNE1 proteins both in the absence of any competitor molecule (FIG. 1O) and in the presence of a significant molar excess of competitor molecule PF-06873600 (FIG. 1P). Incubation of Compound 32 with pCDK2-CCNE1 proteins resulted in covalent complex formation between Compound 32 and the CDK2 protein. The data provided demonstrates two different m/z peaks, which correspond to the mass of the unmodified CDK2 protein and the covalent complex between Compound 32 and CDK2 protein. The peak with specific observed mass value of 34096-34097 Da corresponds to the unmodified CDK2 protein. The second peak to the right of the first with specific observed mass value of 34525 Da corresponds to the covalent complex formed between Compound 32 and CDK2 protein. Thus, as seen in FIG. 1O a noticeable level of covalent complex was formed between Compound 32 and CDK2 protein, indicated by relative intensity of the peak corresponding to the covalent complex compared to those corresponding to the unmodified protein. Furthermore, the extent of covalent complex formation was likely to be even higher than shown in FIG. 1O, as some of the peak intensity for the peak corresponding to unmodified protein was attributable to fragmentation of the C—N bond between Compound 32 and the CDK2 protein (formed by borohydride reduction of the imine bond in the covalent complex) during ionization and detection. In contrast, FIG. 1P demonstrates a decrease in covalent complex formation when pCDK2-CCNE1 proteins were incubated with molar excess competitor molecule PF-06873600, indicated by the relative intensity for the peak corresponding to the unmodified CDK2 protein. Thus, the data provided in FIGS. 1O and 1P show that (1) Compound 32 formed a covalent complex with CDK2 protein, and (2) Compound 32 bound to the same binding pocket of CDK2 as the competitor molecule PF-06873600, as shown by the significant reduction in observed complex formation in the presence of a stoichiometric excess of competitor molecule (compare FIGS. 1O to 1P).

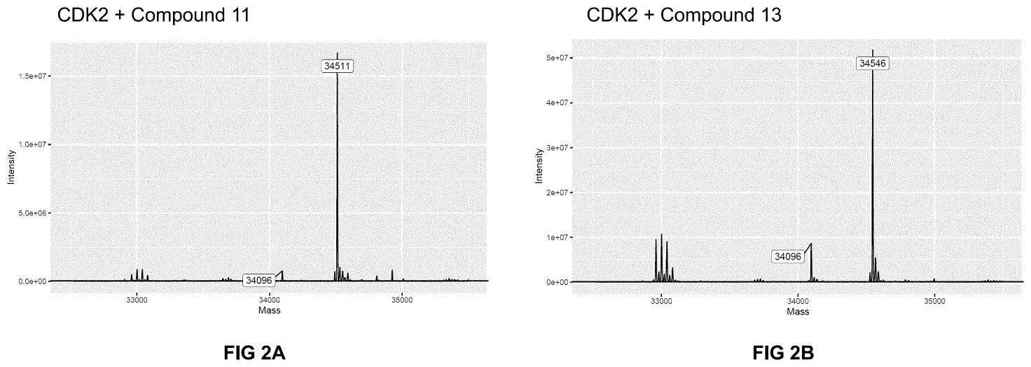

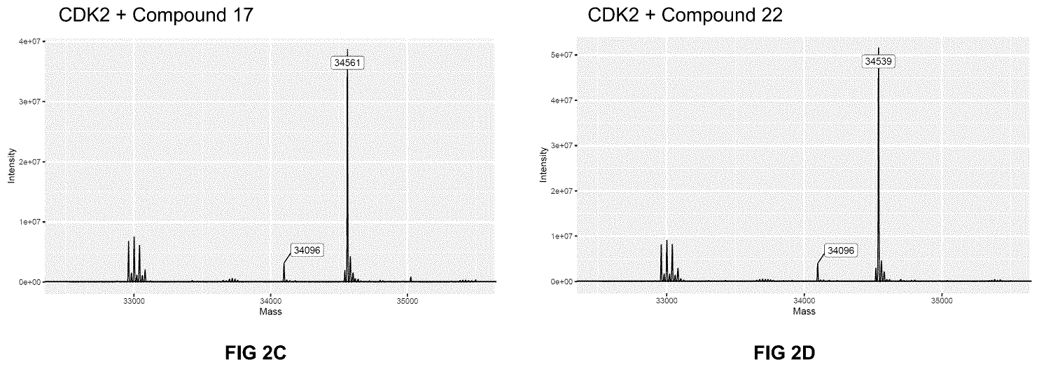

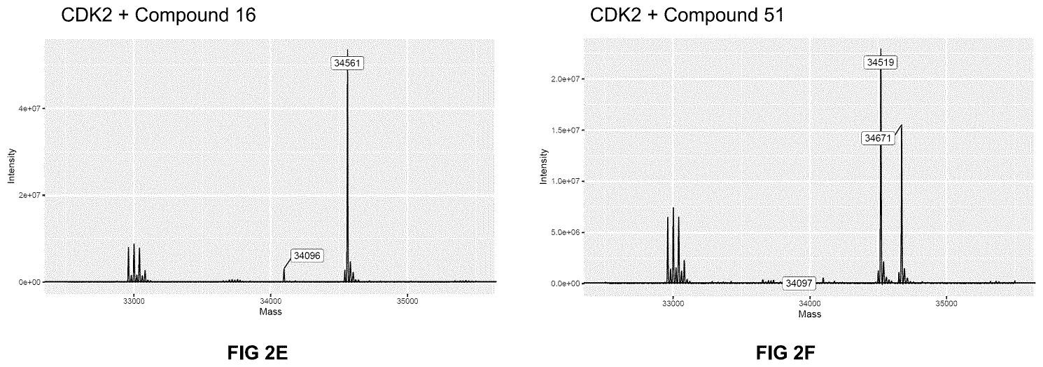

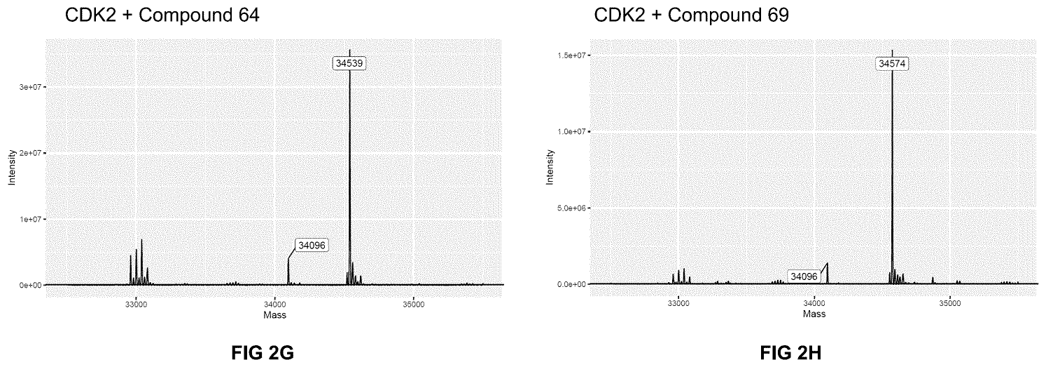

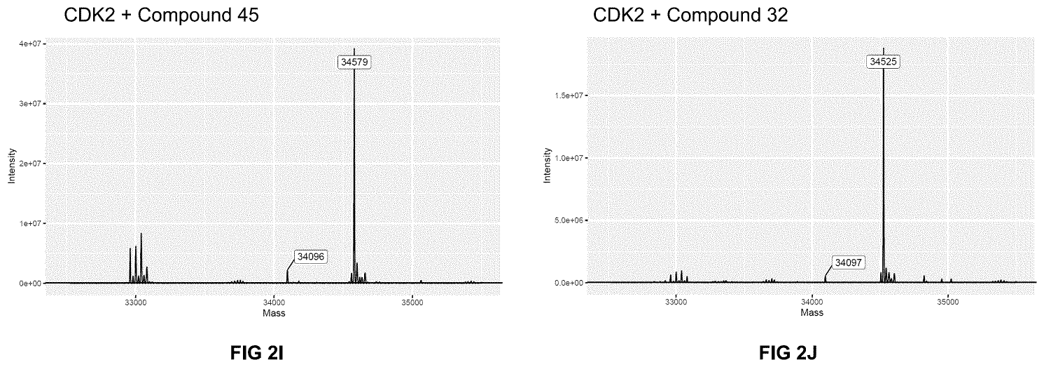

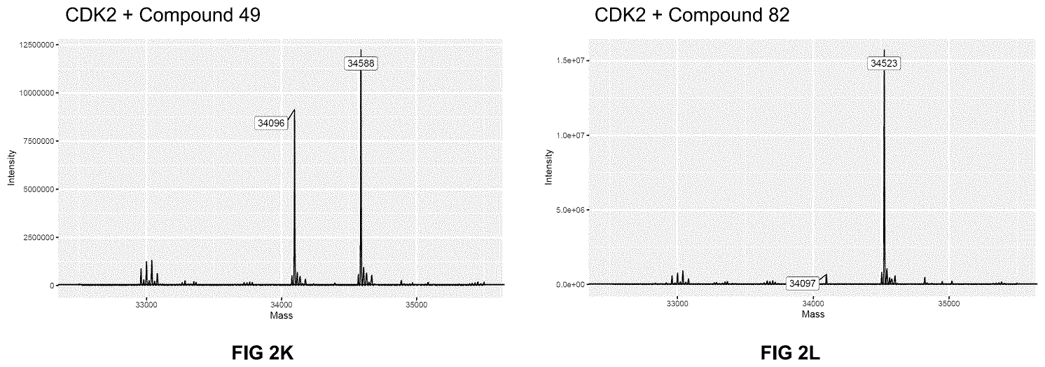

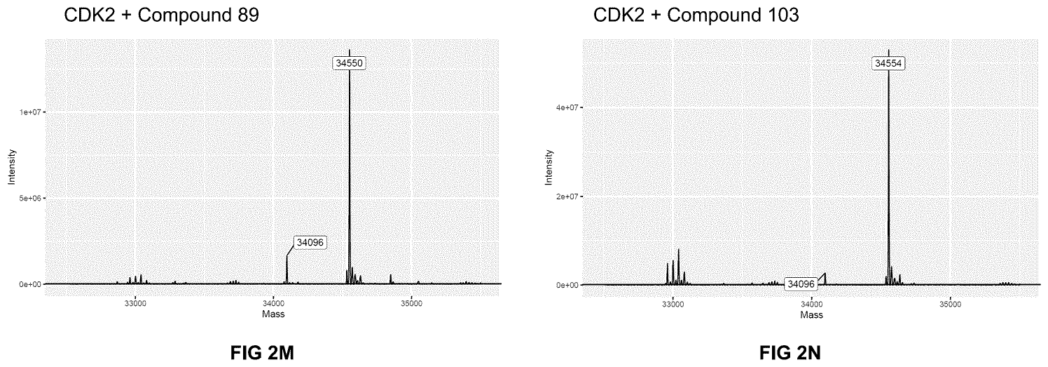

FIGS. 2A to 2N provide intact mass spectra for pCDK2-CCNE1 proteins after incubation with compounds of the present disclosure and subsequent sodium borohydride reduction. Sodium borohydride was added to reduce the reversible imine bond between the pCDK2-CCNE1 proteins and compounds of the present disclosure, thereby forming a more stable C—N single bond between the protein and the compound and allowing detection of a covalent complex by intact mass spectrometry.

FIGS. 2A, 2B, 2C, 2D, 2E, 2F, 2G, 2H, 2J, 2K, 2L, 2M, and 2N provide intact mass spectra following incubation of Compounds 11, 14, 18, 23, 17, 52, 64, 69, 46, 33, 50, 82, 89, and 103, respectively, with pCDK2-CCNE1 proteins. Incubation of the compound with pCDK2-CCNE1 proteins resulted in covalent complex formation between each respective compound and the CDK2 protein. For each compound/CDK2 complex, the data provided demonstrates two different m/z peaks, which correspond to the mass of the unmodified CDK2 protein and the covalent complex between the respective compound and CDK2 protein. The peak with specific observed mass value of 34096-34097 Da corresponds to the unmodified CDK2 protein.

In FIG. 2A, the second peak to the right of the first with specific observed mass value of 34511 Da corresponds to the covalent complex formed between Compound 11 and CDK2 protein. In FIG. 2B, the second peak to the right of the first with specific observed mass value of 34546 Da corresponds to the covalent complex formed between Compound 13 and CDK2 protein. In FIG. 2C, the second peak to the right of the first with specific observed mass value of 34561 Da corresponds to the covalent complex formed between Compound 17 and CDK2 protein. In FIG. 2D, the second peak to the right of the first with specific observed mass value of 34539 Da corresponds to the covalent complex formed between Compound 22 and CDK2 protein. In FIG. 2E, the second peak to the right of the first with specific observed mass value of 34561 Da corresponds to the covalent complex formed between Compound 16 and CDK2 protein. In FIG. 2F, the second peak to the right of the first with specific observed mass value of 34519 Da corresponds to the covalent complex formed between Compound 51 and CDK2 protein. In FIG. 2G, the second peak to the right of the first with specific observed mass value of 34539 Da corresponds to the covalent complex formed between Compound 64 and CDK2 protein. In FIG. 2H, the second peak to the right of the first with specific observed mass value of 34574 Da corresponds to the covalent complex formed between Compound 69 and CDK2 protein. In FIG. 2I, the second peak to the right of the first with specific observed mass value of 34579 Da corresponds to the covalent complex formed between Compound 45 and CDK2 protein. In FIG. 2J, the second peak to the right of the first with specific observed mass value of 34525 Da corresponds to the covalent complex formed between Compound 32 and CDK2 protein. In FIG. 2K, the second peak to the right of the first with specific observed mass value of 34588 Da corresponds to the covalent complex formed between Compound 49 and CDK2 protein. In FIG. 2L, the second peak to the right of the first with specific observed mass value of 34523 Da corresponds to the covalent complex formed between Compound 82 and CDK2 protein. In FIG. 2M, the second peak to the right of the first with specific observed mass value of 34550 Da corresponds to the covalent complex formed between Compound 89 and CDK2 protein. In FIG. 2N, the second peak to the right of the first with specific observed mass value of 34554 Da corresponds to the covalent complex formed between Compound 103 and CDK2 protein.

Thus, as seen in FIGS. 2A to 2N, a noticeable level of covalent complex was formed between each compound and CDK2 protein, indicated by relative intensity of the peaks corresponding to the covalent construct compared to those corresponding to the unmodified protein. Furthermore, the extent of covalent complex formation was likely to be even higher than shown in each of FIGS. 2A to 2N, as some of the peak intensity for the peak corresponding to unmodified protein was attributable to fragmentation of the C—N bond between each respective compound and the CDK2 protein (formed by borohydride reduction of the imine bond in the covalent complex) during ionization and detection.

Dissociation Rate Data for Compounds with pCDK2-CCNE1

pCDK2-CCNE1 proteins (1 μM) and were treated with a compound (1.5 μM) from Table 4 for 1 hour at room temperature or at 37° C., then competitor molecule PF-06873600 (20 μM or 50 μM final concentration) was added to prevent aldehyde containing compound rebinding after initial dissociation. At indicated timepoints, samples were aliquoted and incubated with 5 mM sodium borohydride at room temperature for 5 min to capture reversible imines through reductive amination. Reduction reactions were quenched by diluting the sample in equal volume of 1% trifluoroacetic acid (v/v) in acetonitrile, then analyzed by LC-MS. Deconvoluted monoisotopic intensities of unmodified and compound-modified mass peaks were compared.

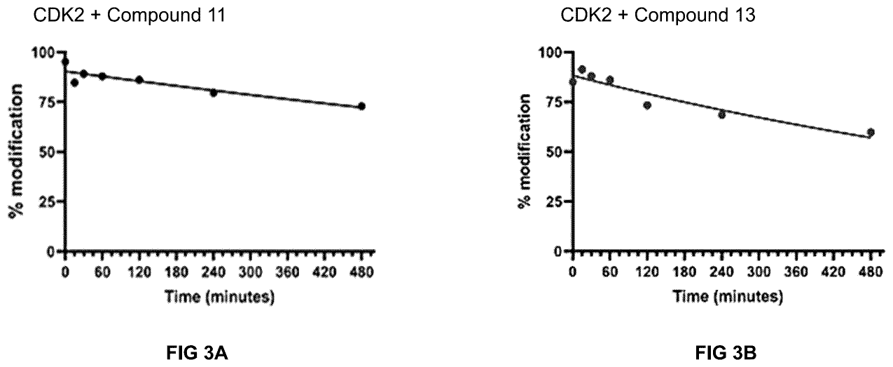

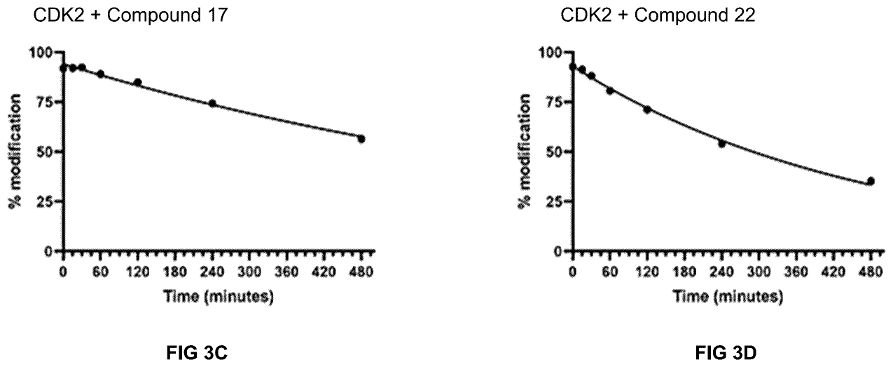

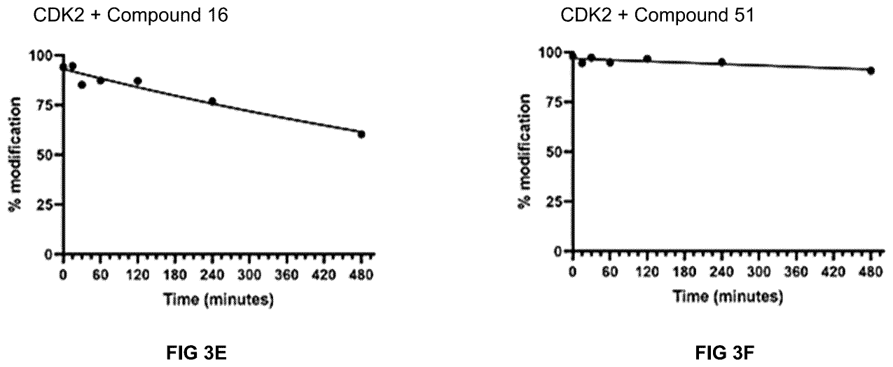

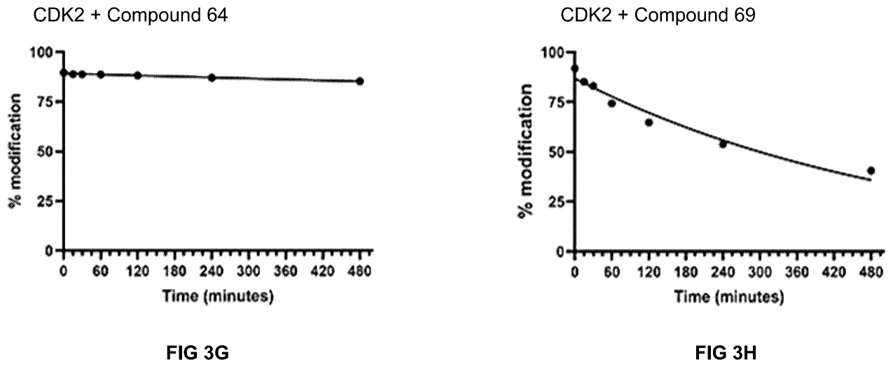

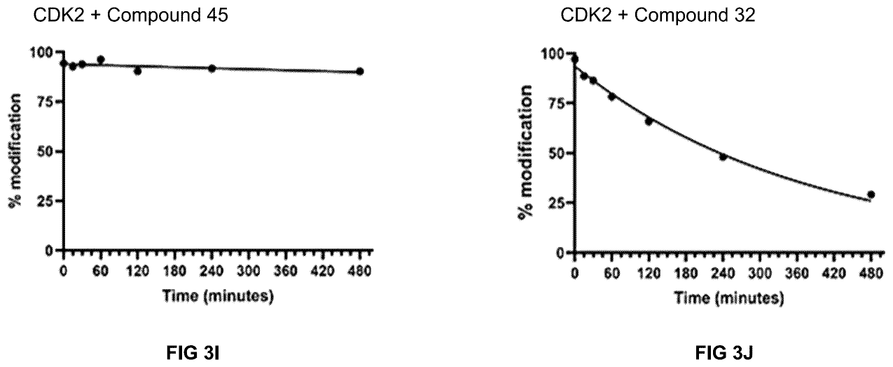

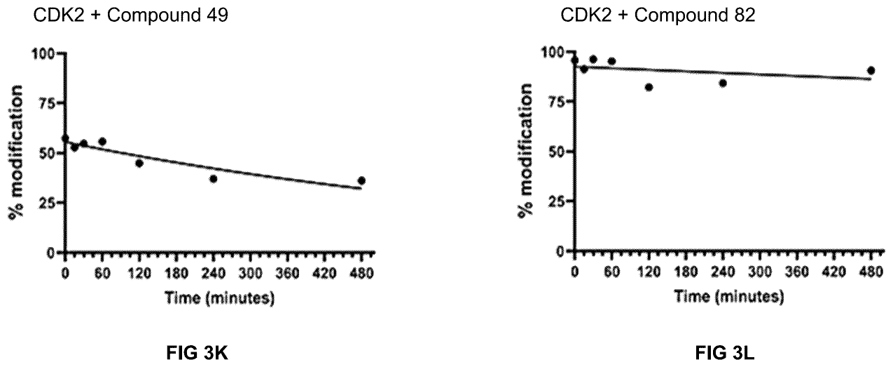

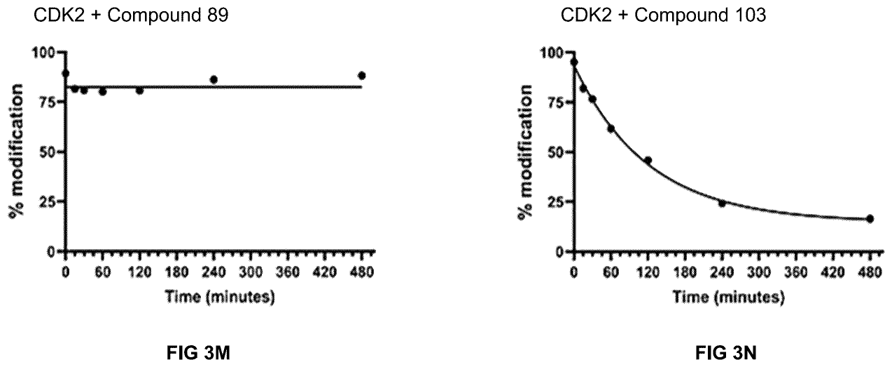

FIGS. 3A to 3N illustrate off-rate determination studies of compounds of the present disclosure from pCDK2-CCNE1 determined by intact mass spectrometry. FIGS. 3A, 3B, 3C, 3D, 3E, 3F, 3G, 3H, 3I, 3J, 3K, 3L, 3M, and 3N provide dissociation rate data from pCDK2-CCNE1 treated with Compounds 11, 14, 18, 23, 17, 52, 64, 69, 46, 33, 50, 82, 89, and 103, respectively, via competition with molar excess competitor molecule PF-06873600. Table 3 provides the half-life data for the dissociation of pCDK2-CCNE1 treated with each respective compound.

TABLE 3
Half-life data for compounds

| | CDK2/ | | CDK2/ |
|---|---|---|---|
| | CyclinE1 t_{1/2} | | CyclinE1 t_{1/2} |
| Compound No. | (mins) | Compound No. | (mins) |
| 11 | 1490 | 141 | 460 |
| 13 | 763 | 145 | 664 |
| 17 | 679 | 195 | 5080 |
| 22 | 325 | 199 | 111 |
| 16 | 804 | 204 | 1960 |
| 51 | 5730 | 207 | 2280 |
| 64 | 7220 | 208 | 1700 |
| 69 | 376 | 209 | 3260 |
| 45 | 7530 | 215 | 1440 |
| 32 | 260 | 218 | 1870 |
| 49 | 608 | 219 | 2710 |
| 82 | 4800 | 220 | 1920 |
| 89 | >10 000 | 221 | 1780 |
| 103 | 83.6 | 222 | 1090 |
| 110 | 364 | 223 | 1820 |
| 111 | 1820 | 224 | 935 |
| 112 | 395 | 227 | 645 |
| 115 | 3340 | 228 | 1870 |
| 229 | 3190 | 245 | 2260 |
| 230 | 1340 | 247 | 2140 |
| 231 | 2090 | 248 | 5230 |
| 232 | 2080 | 249 | 8760 |
| 235 | 1190 | 250 | 517 |
| 239 | 7490 | 251 | 3540 |
| 241 | 1080 | 253 | 1330 |
| 242 | 1270 | 254 | 15400 |
| 243 | 5370 | 255 | 1130 |
| 244 | 3020 | 256 | 46.3 |

### Example 42 Crystallography Studies Showing Covalent Complex Formation

### Protein Expression and Purification

The pCDK2-CCNE1 complex was produced by co-expressing CCNE1 and CDK2 together with the kinase CAKI inSf21 cells using BVES at 300K for 66 hours. The cell pellet was stored at 193 K until lysis. Both complex partners contain an N-terminal His tag followed by a Sumo tag, while CAKI contains an N-terminal His-Strep-TEV-tag.

The cells were lysed in buffer A (20 mM HEPES/NaOH, 300 mM NaCl, 20 mM imidazole, 5% glycerol, 1 mM TCEP, pH 7.5) supplemented with 0.1% NP-40, 0.2 mM MgCl₂, 1 mM ATP, Benzonase and protease inhibitor tablets using a Microfluidizer LM10.

Purification was performed by Ni-NTA capture of the His-tagged proteins. After washing with buffer A, the pCDK2-CCNE1 complex was eluted from the IMAC resin with buffer A supplemented with 400 mM imidazole. The His-Sumo tag was cleaved from CCNE1 and CDK2 by ULP1 protease and removed from the sample together with uncleaved protein and CAKI by a reverse IMAC purification step in buffer B (20 mM HEPES/NaOH, 150 mM NaCl, 5% glycerol, 1 mM TCEP, pH 7.5). As final purification step, the pCDK2-CCNE1 complex was polished by size-exclusion chromatography on a S200 PG column, equilibrated in buffer C (20 mM Tris/HCl, 150 mM NaCl, 1 mM TCEP, pH 8.0). The protein was then concentrated to 10 mg/mL using a Vivaspin Turbo concentrator and stored in small aliquots at 193 K until crystallization.

### Crystallization, Data Collection and Structure Determination

Crystallization of apo pCDK2-CCNE1 in ligand free state: Crystals were obtained by the vapor diffusion technique at 20° C. after mixing an equal volume of the pCDK2-CCNE1 complex (8-10 mg/ml) with the well solution containing 1.6-2.4M NaCl, 100 mM Tris (pH 7.0-8.0). Crystals grew within 3 days.

Soaking: The apo crystals were soaked in mother liquid containing 1 mM of a compound of interest overnight, then cryoprotected in mother liquor mixed with equal volume of 7M Sodium Formate before cryocooled in liquid nitrogen.

Diffraction datasets of compound 13 and compound 17 were collected and processed by Helix Biostructures at Advanced Photon Source (APS) 231D-B beamline. Diffraction datasets of compound 64 were collected and processed by Helix Biostructures at Canadian Light Source (CLS) CMCF 08ID-1 beamline on 2023 May 12.

These datasets were further processed with xia2 (ccp4). The initial structures were determined through molecular replacement with Phaser in CCP4 suite with the structure of PDB ID 1W98 as a search model. A chemical restraint dictionary was generated using ACEDRG and CCP4i2. The models were manually adjusted using Coot, refined with REFMAC5 to a final resolution of 1.9 Å for compound 13 and compound 17 complexes, and 2.0 Å for the compound 64 complex.

[TABLE 4
Data collection and refinement statistics

| | PCDK2- | PCDK2- | PCDK2-CCNE1/ |
|---|---|---|---|
| | CCNE1/Compound 13 | CCNE1/Compound 17 | Compound 64 |
| Wavelength | 1.033 | 1.033 | 0.9537 |
| Resolution range | 40.47-1.9 | 38.03-1.91 | 63.83-2.0 |
| | (1.968-1.9) | (1.978-1.91) | (2.071-2.0) |
| Space group | P 32 2 1 | P 32 2 1 | P 32 2 1 |
| Unit cell | 76.0215 | 76.06 | 76.06 |
| | 76.0215 | 76.06 | 76.06 |
| | 256.531 | 257.517 | 258.19 |
| | 90 | 90 | 90 |
| | 90 | 90 | 90 |
| | 120 | 120 | 120 |
| Total reflections | 761872 | 747309 | 660567 (67332) |
| | (71089) | (74814) | |
| Unique reflections | 68940 | 68203 | 59701 |
| | (6752) | (6655) | (5840) |
| Multiplicity | 11.1 | 11.0 | 11.1 |
| | (10.5) | (11.2) | (11.5) |
| Completeness (%) | 99.85 | 99.72 | 99.98 |
| | (99.88) | (99.13) | (99.95) |
| Mean I/sigma(I) | 14.39 | 9.09 | 8.52 |
| | (0.95) | (0.83) | (1.08) |
| Wilson B-factor | 39.48 | 39.18 | 36.06 |
| R-merge | 0.0862 | 0.1256 | 0.1854 |
| | (2.348) | (2.859) | (2.22) |
| R-meas | 0.09039 | 0.1319 | 0.1945 |
| | (2.469) | (2.996) | (2.324) |
| R-pim | 0.02696 | 0.03976 | 0.05808 |
| | (0.7576) | (0.891) | (0.6825) |
| CC1/2 | 1 | 0.998 | 0.997 |
| | (0.403) | (0.357) | (0.37) |
| CC* | 1 | 1 | 0.999 |
| | (0.758) | (0.726) | (0.735) |
| Reflections used in | 68848 | 68048 | 59695 |
| refinement | (6744) | (6617) | (5837) |
| Reflections used for R-free | 3382 | 3363 | 2948 |
| | (375) | (321) | (312) |
| R-work | 0.2103 | 0.2176 | 0.2210 |
| | (0.3614) | (0.3791) | (0.4075) |
| R-free | 0.2524 | 0.2595 | 0.2574 |
| | (0.3617) | (0.4127) | (0.4486) |
| CC(work) | 0.955 | 0.952 | 0.954 |
| | (0.642) | (0.615) | (0.588) |
| CC(free) | 0.937 | 0.942 | 0.933 |
| | (0.601) | (0.492) | (0.405) |
| Number of non-hydrogen | 5277 | 5300 | 5267 |
| atoms | | | |
| macromolecules | 4909 | 4914 | 4921 |
| ligands | 80 | 81 | 79 |
| solvent | 288 | 305 | 267 |
| Protein residues | 573 | 573 | 573 |
| RMS(bonds) | 0.011 | 0.006 | 0.011 |
| RMS(angles) | 1.49 | 0.90 | 1.43 |
| Ramachandran favored (%) | 95.03 | 96.98 | 95.38 |
| Ramachandran allowed (%) | 4.09 | 2.66 | 3.91 |
| Ramachandran outliers (%) | 0.89 | 0.36 | 0.71 |
| Rotamer outliers (%) | 3.80 | 2.35 | 3.06 |
| Clashscore | 9.48 | 7.21 | 6.01 |
| Average B-factor | 52.22 | 55.51 | 49.76 |
| macromolecules | 52.26 | 55.70 | 49.85 |
| ligands | 67.40 | 67.22 | 63.74 |
| solvent | 47.26 | 49.36 | 44.02 |

Statistics for the highest-resolution shell are shown in parentheses.

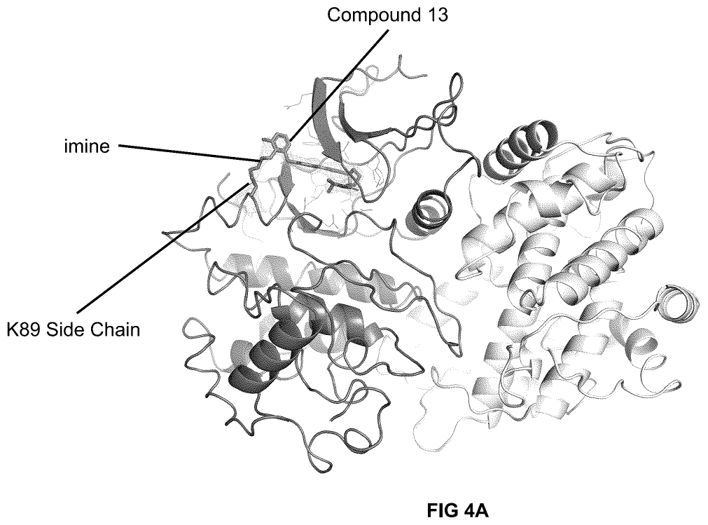

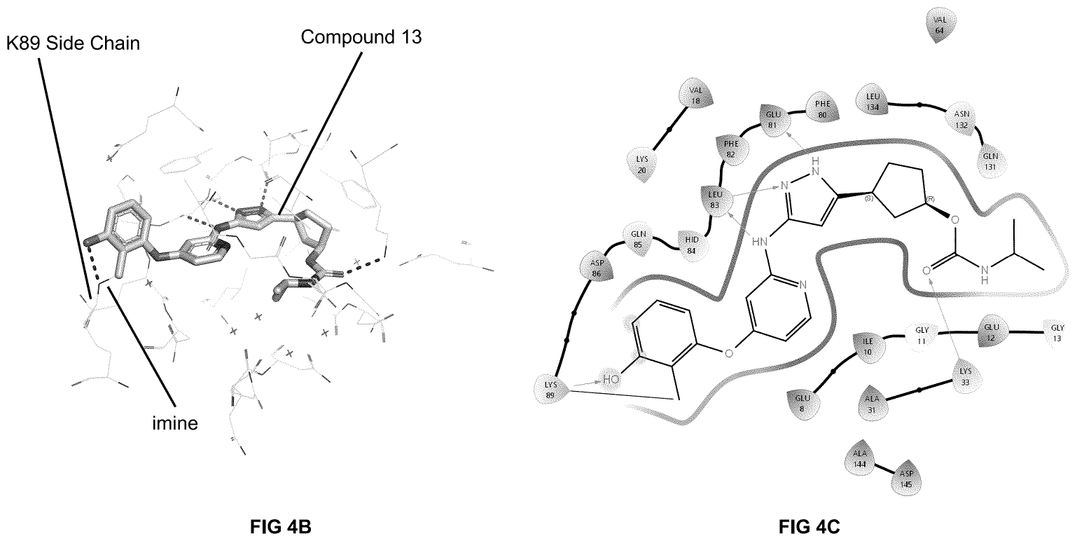

FIGS. 4A to 4C provide crystal structure representations for the co-crystallization of Compound 13 and pCDK2-CCNE1. In FIG. 4A, the pCDK2-CCNE1-Compound 13 co-crystallization complex is shown. For the 2Fo-Fc maps contoured at 1.06, the pCDK2-CCNE1 complex is shown in cartoon rending and Compound 13 with the side chain of K89 of CDK2 are represented by a thicker stick model. The electron density indicates covalent bond formation with K89 and CDK2. FIG. 4B provides a close-up from the crystal structure of the co-crystallization of Compound 13 and pCDK2-CCNE1. In FIG. 4B, Compound 13 is represented by a thicker stick model and the adjacent residues of CDK2, including the side chain of K89, are represented by a thinner stick model. Hydrogen bonds are depicted by dashed lines. FIG. 4B shows the imine bond formed between the nitrogen of K89 and the aldehyde of Compound 13, as well as the hydrogen bond between the imine nitrogen lone pair and the adjacent phenol hydrogen. The crystal structure provided in FIG. 4B thereby depicts the formation of a covalent complex between Compound 13 and K89 of CDK2. FIG. 4C provides a 2-D diagram detailing the interactions between Compound 13 and residues of the CDK2 protein. In FIG. 4C, the imine bond is depicted by the line between K89 and the methylene of Compound 13. Additionally, hydrogen bonds are depicted as arrows between Compound 13 and the residues of the CDK2 protein.

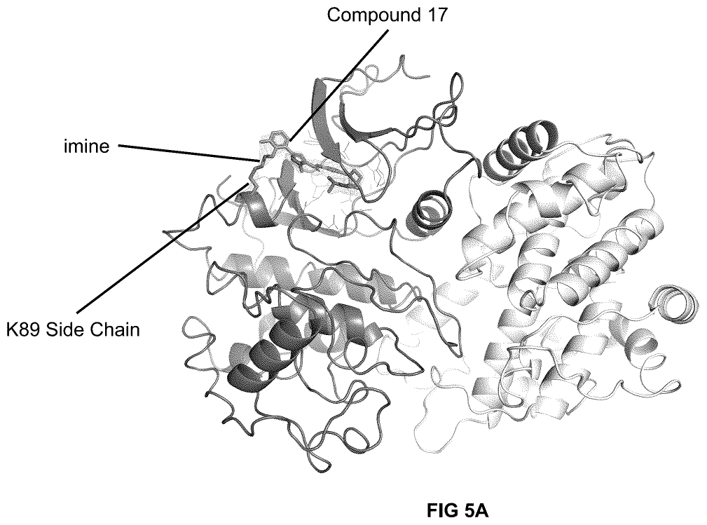

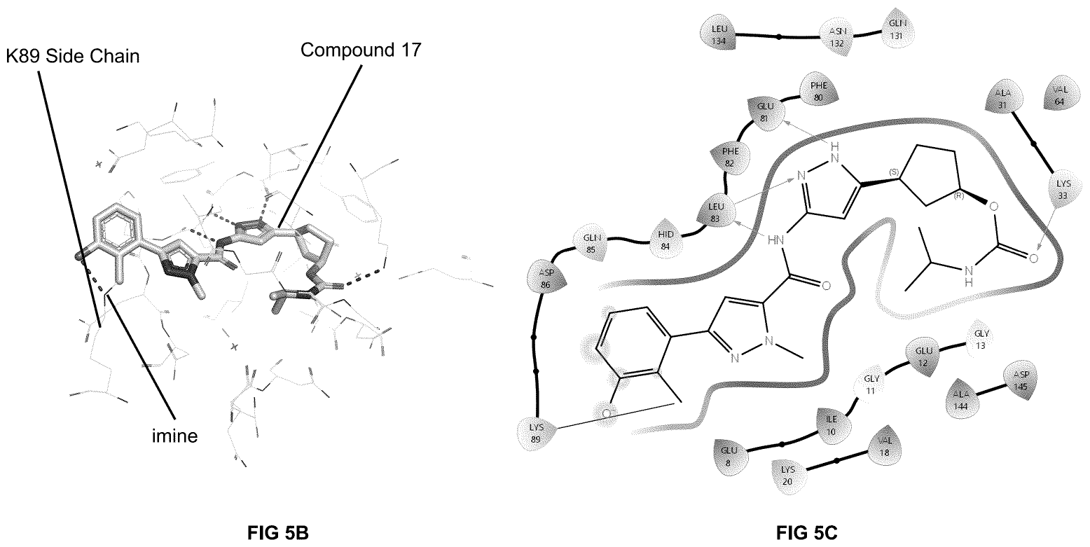

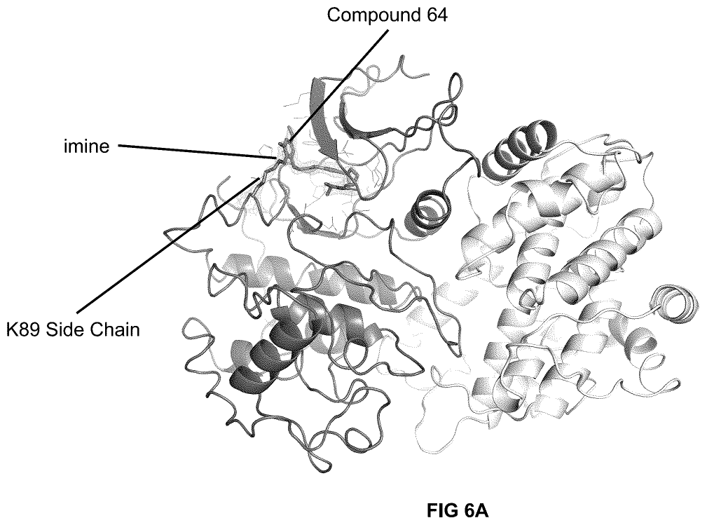

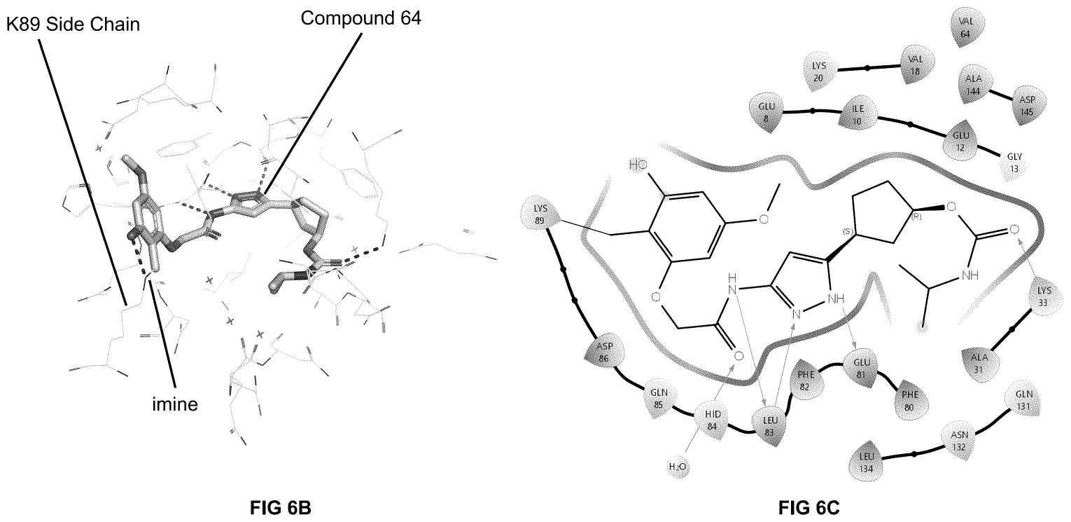

FIGS. 5A to 5C provide crystal structure representations for the co-crystallization of Compound 17 and pCDK2-CCNE1. In FIG. 5A, the pCDK2-CCNE1-Compound 17 co-crystallization complex is shown. For the 2Fo-Fc maps contoured at 1.0, the pCDK2-CCNE1 complex is shown in cartoon rending and Compound 17 with the side chain of K89 of CDK2 are represented by a thicker stick model. The electron density indicates covalent bond formation with K89 and CDK2. FIG. 5B provides a close-up from the crystal structure of the co-crystallization of Compound 17 and pCDK2-CCNE1. In FIG. 5B, Compound 17 is represented by a thicker stick model and the adjacent residues of CDK2, including the side chain of K89, are represented by a thinner stick model. Hydrogen bonds are depicted by dashed lines. FIG. 5B shows the imine bond formed between the nitrogen of K89 and the aldehyde of Compound 17, as well as the hydrogen bond between the imine nitrogen lone pair and the adjacent phenol hydrogen. The crystal structure provided in FIG. 4B thereby depicts the formation of a covalent complex between Compound 17 and K89 of CDK2. FIG. 5C provides a 2-D diagram detailing the interactions between Compound 17 and residues of the CDK2 protein. In FIG. 5C, the imine bond is depicted by the line between K89 and the methylene of Compound 17. Additionally, hydrogen bonds are depicted as arrows between Compound 17 and the residues of the CDK2 protein. FIGS. 6A to 6C provide crystal structure representations for the co-crystallization of Compound 64 and pCDK2-CCNE1. In FIG. 6A, the pCDK2-CCNE1-Compound 64 co-crystallization complex is shown. For the 2Fo-Fc maps contoured at 1.00, the pCDK2-CCNE1 complex is shown in cartoon rending and Compound 64 with the side chain of K89 of CDK2 are represented by a thicker stick model. The electron density indicates covalent bond formation with K89 and CDK2. FIG. 6B provides a close-up from the crystal structure of the co-crystallization of Compound 64 and pCDK2-CCNE1. In FIG. 6B, Compound 64 is represented by a thicker stick model and the adjacent residues of CDK2, including the side chain of K89, are represented by a thinner stick model. Hydrogen bonds are depicted by dashed lines. FIG. 6B shows the imine bond formed between the nitrogen of K89 and the aldehyde of Compound 64, as well as the hydrogen bond between the imine nitrogen lone pair and the adjacent phenol hydrogen. The crystal structure provided in FIG. 4B thereby depicts the formation of a covalent complex between Compound 64 and K89 of CDK2. FIG. 6C provides a 2-D diagram detailing the interactions between Compound 64 and residues of the CDK2 protein. In FIG. 6C, the imine bond is depicted by the line between K89 and the methylene of Compound 64. Additionally, hydrogen bonds are depicted as arrows between Compound 64 and the residues of the CDK2 protein.

### Example 43: Prodrug Biodata

### Example 43.1: Solubility Determination in PBS pH 7.4

Stock solutions (10 mM DMSO, 30 μL) of each sample were placed into 96-well plate, along with 970 μL of PBS pH 7.4 were added into each vial of the cap-less solubility sample plate. The assay was performed in duplicate. A stir stick was added to each well and sealed using a molded PTFE/Silicone plug. Then the Solubility Sample plate was transferred to an Eppendorf Thermomixer Comfort plate shaker and shaken at 1100 RPM for 2 hours at 25° C. After completion the plugs and stir sticks were removed and the samples were transferred into a filter plate and filtered using a vacuum manifold. 5 μL aliquots of filtrate were taken and diluted with 5 μL DMSO and 490 μL of a 1:1 v/v mixture of H₂O and acetonitrile. 200 μL of diluent was transferred to a new 96-well plate for LC-MS/MS analysis. The solution was analyzed and quantified against a standard of known concentration in DMSO using LC coupled with Mass spectral peak for identification and quantitation.

### Example 43.2: Oral Bioavailability Determination and Pharmacokinetic Analysis Following Oral Administration in Nu/Nu Mice

Compound was administered via oral gavage in male Nu/Nu mice (n=3). The vehicle composition was 10% N-Methylpyrrolidone, 40% PEG300, 50% water (pH=4-8) at 6 mg-equivalent/mL to parent drug and a dose volume of 5 mL/Kg. Formulations were prepared freshly before dosing and stored at room temperature. Plasma samples were collected at the following time intervals: 0.5, 1, 2, 4, 8, 12, 24, 48, and 72 hours post dose (based on the start of oral dosing). Serial blood samples were collected (approximately 0.025 mL) via retro-orbital vein into K₂EDTA. Whole blood was processed into plasma by centrifuge (4000 rpm for 5 minutes at 4° C.) followed by addition of 0.5 M ammonium formate (pH=3, 300% of plasma volume). Plasma samples were stored in a freezer at −75±15° C. until analysis. Plasma samples were analyzed after thawing and incubating for 1 h at 37° C. followed by protein precipitation with acetonitrile containing IS mixture (vortex 30 s, centrifugation at 4° C., 400 rpm for 15 min) and the resulting supernatant was further diluted with water before injecting onto Shimadzu 8060 LC-MS/MS system for analysis. Unknown plasma samples concentration was calibrated using standard range of 0.2 to 1 000 ng/mL for parent.

Bioavailability (% F) was calculated by comparing observed plasma concentrations of parent drug after oral administration of prodrug or parent drug to observed plasma concentrations via IV (intravenous) administration of parent drug.

$$\% F = [ PO AUCinf \cdot IV Dose ) / ( IV AUCinf \cdot PO Dose ) ] \cdot 100 \% .$$

TABLE 5
Solubility, pharmacokinetic, and bioavailability
data for selected compounds.

| | Prodrug | Parent | | | | |
|---|---|---|---|---|---|---|
| | Sol- | Sol- | | | | |
| | ubility | ubility | Prodrug | Parent | Prodrug | Parent |
| Pro- | pH 7.4 | pH 7.4 | AUC_{inf} | bAUC_{inf} | Mouse | Mouse |
| drug | (μM) | (μM) | (μM · h) | (μM · h) | % F | % F |
| 128b | | 2.8 | 210 | 5.7 | 54.9 | 1.5 |
| 172a | 227 | 93 | 53.9 | 40.2 | 14.7 | 11 |
| 251b | 41 | 0.4 | 73 | | 22.7 | |
| 251d | | 0.4 | 3.4 | | 1 | |
| 251c | 48 | 0.4 | 135 | | 41.8 | |
| 64d | 67 | 0.7 | 83.7 | 0.3 | 16.7 | 0.05 |
| 64e | | 0.7 | 3.6 | 0.3 | 0.7 | 0.05 |
| 145a | 68 | 0.6 | 176 | 0.2 | 63.6 | 0.08 |
| 64f | | 0.7 | 15.9 | 0.3 | 3.1 | 0.05 |
| 64g | | 0.7 | 13.6 | 0.3 | 2.7 | 0.05 |
| 64h | | 0.7 | 42.6 | 0.3 | 8.4 | 0.05 |
| 64i | | 0.7 | 1.22 | 0.3 | 0.2 | 0.05 |
| 64j | | 0.7 | 11.7 | 0.3 | 2.3 | 0.05 |
| 64k | | 0.7 | 5.4 | 0.3 | 1.1 | 0.05 |
| 64l | | 0.7 | 42.4 | 0.3 | 8.1 | 0.05 |
| 64b | 79 | 0.7 | 159 | 0.3 | 31 | 0.05 |
| 64m | | 0.7 | 72 | 0.3 | 9.9 | 0.05 |
| 64n | | 0.7 | 34.4 | 0.3 | 6.8 | 0.05 |
| 64o | | 0.7 | 33.3 | 0.3 | 6.6 | 0.05 |
| 128a | 191 | 2.8 | 220 | 5.7 | 60.8 | 1.5 |
| 254a | | 0.4 | 78 | | 20.6 | |
| 64c | 246 | 0.7 | 123 | 0.3 | 24.2 | 0.05 |
| 64p | 4.8 | 0.7 | 5.4 | 0.3 | 1.06 | 0.05 |
| 64q | 243 | 0.7 | 2.5 | 0.3 | 0.5 | 0.05 |
| 235a | | 27 | 15.2 | | 3.2 | |
| 224a | | 21 | 57 | | 20.6 | |
| 180a | | 7.0 | 53.6 | | 15.7 | |
| 253a | | 2.3 | 48.5 | | 9.5 | |
| 82a | 34 | 0.8 | 114 | 1.1 | 33 | 0.3 |
| 221a | 156 | 1.3 | 123 | | 30.6 | |
| 223a | 6.7 | 0.7 | 90.7 | 0.9 | 14.2 | 0.1 |
| 251a | 50 | 0.4 | 184 | | 56.7 | 0.04 |
| 64a | 215 | 0.7 | 144 | 0.3 | 31 | 0.05 |
| 231a | 158 | 35 | 12.9 | 18.8 | 2.8 | 2.7 |
| 283a | 149 | 18.1 | | | | |
| 284a | 253 | 152 | | | | |
| 284c | | 152 | | | | |
| 284b | | 152 | | | | |

### Example 43.3: Pharmacokinetic Analysis Following Oral and IV Administration of Compound 64a to Nu/Nu Mice

Compound 64a was administered via oral gavage or IV in male Nu/Nu mice (n=3). The vehicle composition for oral dosing was 10% N-Methylpyrrolidone, 40% PEG300, 50% water (pH=6-7) at 6 mg-equivalent/mL to parent drug and a dose volume of 5 mL/Kg. The vehicle composition for IV dosing was 10% N-Methylpyrrolidone, 40% PEG300, 50% water (pH=6-7) at 1 mg/mL prodrug and a dose volume of 5 mL/Kg. Formulations were prepared freshly before dosing and stored at room temperature. Plasma samples were collected at the following time intervals: 0.5, 1, 2, 4, 8, 12, 24, 48, and 72 hours post dose. Serial blood samples were collected (approximately 0.025 mL) via retro-orbital vein and via heart puncture for final time point into K₂EDTA. Whole blood was processed into plasma by centrifuge (4000 rpm for 5 minutes at 4° C.), approximately half of isolated plasma was stored without further manipulation, remaining plasma was diluted with 0.5 M ammonium formate (pH=3,300% of plasma volume). Plasma samples were stored in a freezer at −75±15° C. until analysis. Plasma samples were thawed and samples treated with ammonium formate were incubated for additional 1 h at 37° C. Plasma samples were precipitated with acetonitrile containing IS mixture (vortex 30 s, centrifugation at 4° C., 4000 rpm for 15 min) and the resulting supernatant was further diluted with water before injecting onto Shimadzu 8060 LC-MS/MS system for analysis. Samples treated with ammonium formate were analyzed for compound 64, and samples not treated with ammonium formate were analyzed for compound 64a. Unknown plasma samples concentration was calibrated using standard range of 0.2 to 1 000 ng/mL for parent.

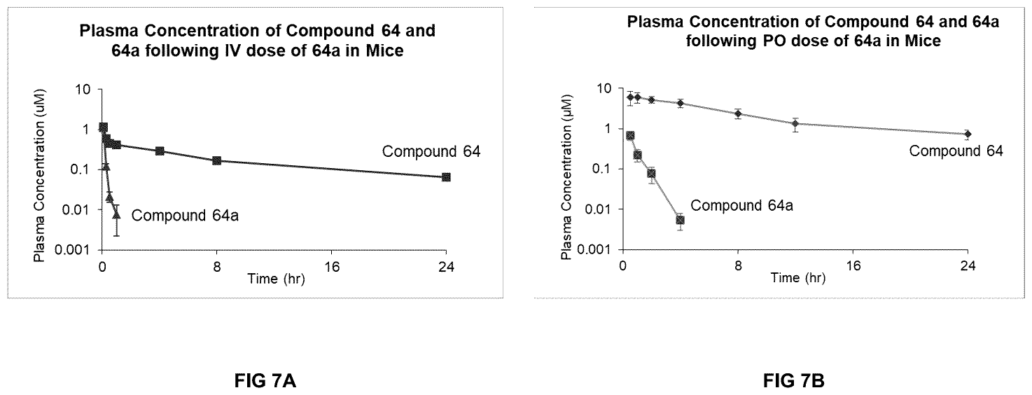

FIGS. 7A-7B provide plasma concentrations for compounds 64 and 64a following oral and IV administration to mice. FIG. 7A shows plasma concentrations for compounds 64 and 64a following oral administration to mice. FIG. 7B shows plasma concentrations for compounds 64 and 64 following IV administration to mice.

What is claimed is:

1. A compound represented by the structure of Formula (II): 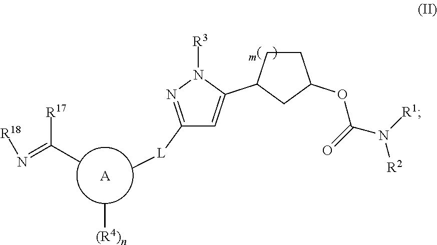
or a pharmaceutically acceptable salt thereof wherein:
Ring A is selected from C₃₋₁₂ carbocycle and 3- to 12-membered heterocycle;
each R⁴ is independently selected at each occurrence from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₃₋₁₀ carbocycle and 3- to 10-membered heterocycle, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
L is represented by -L¹-L²-L³-L⁴-, wherein L¹, L², L³, and L⁴ are each independently selected from (a) and (b):
(a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
(b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN;
wherein L², L³, and L⁴ are each optionally absent;
wherein no more than two of L¹, L², L³, and L⁴ are selected from (a) and the two selected are not adjacent;
R¹ and R² are each independently selected from (i), (ii), and (iii):
(i) hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, —N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN;
(ii) C₁₋₆ alkyl, C₂₋₆ alkenyl, and C₂₋₆ alkynyl, each of which is optionally substituted with one or more substituents independently selected from:
halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
C₃₋₁₀ carbocycle and 3- to 10-membered heterocycle, each of which is optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
(iii) C₃₋₁₀ carbocycle and 3- to 10-membered heterocycle, each of which is optionally substituted with one or more substituents independently selected from: halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl; the C₁₋₆ alkyl, C₂₋₆ alkenyl, and C₂₋₆ alkynyl are each optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; or
R¹ and R² may come together to form a 3- to 10-membered heterocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN; and
C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN;
R³ is selected from hydrogen, C₁₋₆ alkyl, and C₁₋₆ haloalkyl;
R¹¹, R¹², R¹³, R¹⁴, R¹⁵, and R¹⁶ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from halogen, —OH, —O—C₁₋₆ alkyl, —O—C₁₋₆ haloalkyl, —NH₂, —NO₂, ═O, and —CN; and
C₃₋₆ carbocycle and 3- to 6-membered heterocycle optionally substituted with one or more substituents independently selected from halogen, —OH, —O—C₁₋₆ alkyl, —O—C₁₋₆ haloalkyl, —NH₂, —NO₂, ═O, and —CN;
R¹⁷ is selected from hydrogen, C₁₋₆ alkyl, and C₁₋₆ haloalkyl;
R¹⁸ is selected from:
hydrogen;
C₁₋₆ alkyl optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —P(═O)(OR²¹)₂, —NO₂, ═O, ═S, ═N(R²¹), —CN, C₃₋₁₀ carbocycle, and 3- to 10-membered heterocycle; and
C₃₋₁₀ carbocycle and 3- to 10-membered heterocycle, any of which are optionally substituted with one or more substituents independently selected from halogen, —OR²¹, —SR²¹, —N(R²¹)₂, —C(O)R²¹, —C(O)N(R²¹)₂, —C(O)OR²¹, —OC(O)R²¹, —N(R²¹)C(O)OR²¹, —N(R²¹)C(O)R²¹, —S(O)R²¹, —S(O)₂R²¹, —N(R²¹)S(O)₂R²¹, —S(O)₂N(R²¹)₂, —NO₂, ═O, ═S, ═N(R²¹), —CN, C₁₋₆ alkyl, and C₁₋₆ haloalkyl;
R²⁰ and R²¹ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₆ carbocycle and 3- to 6-membered heterocycle optionally substituted with one or more substituents independently selected from: halogen, —OH, —O—C₁₋₆ alkyl, —O—C₁₋₆ haloalkyl, —NH₂, —NO₂, ═O, and —CN.
n is selected from 0, 1, 2, 3, and 4; and
m is selected from 0 and 1.

2. The compound or salt of claim 1, wherein R¹⁷ is hydrogen.

3. The compound or salt of claim 1, wherein R¹⁸ is selected from hydrogen, C₁₋₆ alkyl, and C₁₋₆ haloalkyl.

4. The compound or salt of claim 1, wherein R¹⁸ is selected from hydrogen,

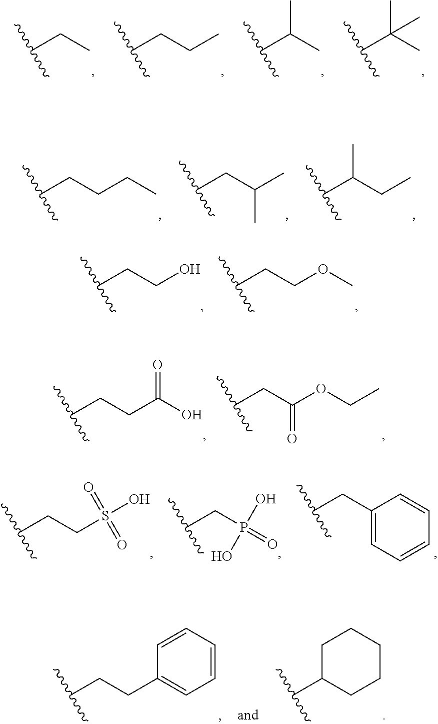

5. The compound or salt of claim 2, wherein 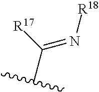
is selected from

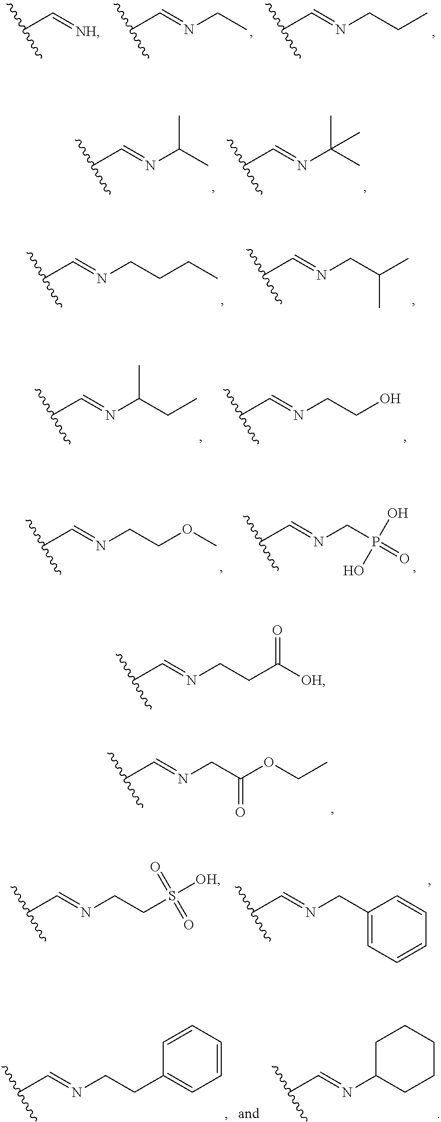

6. The compound or salt of claim 1, wherein 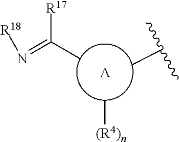
is selected from

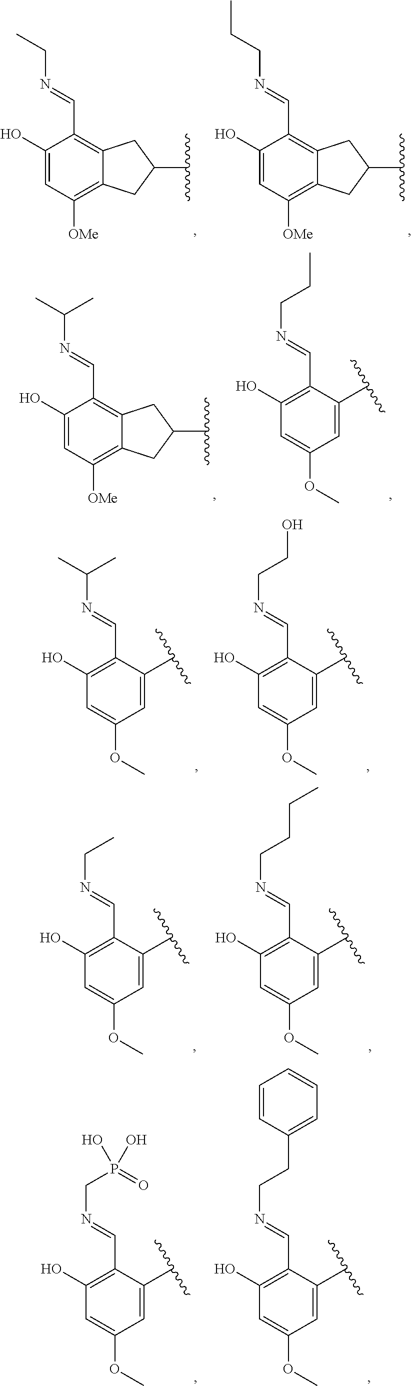

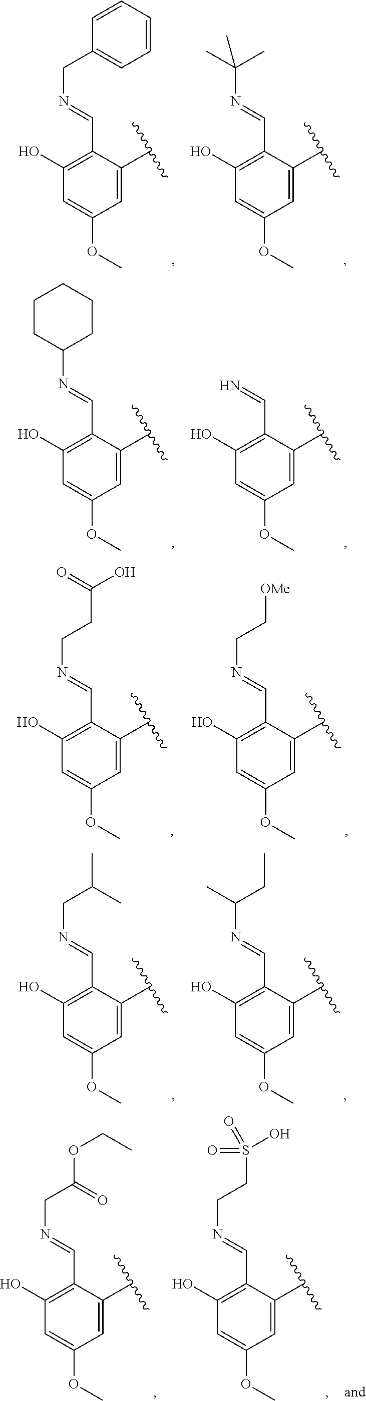

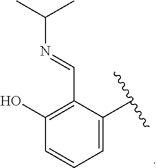 [SMILES: *c1cccc(O)c1/C=N/C(C)C]

7. The compound or salt of claim 1, wherein one of R¹ and R² is selected from hydrogen, C₁₋₆ alkyl, and C₁₋₆ haloalkyl, and the other is selected from:
—OR¹⁴, —N(R¹⁴)₂, —C(O)R¹⁴, —C(O)N(R¹⁴)₂, —CN and, C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from halogen, —OR¹⁴, —N(R¹⁴)₂, —C(O)R¹⁴, —C(O)N(R¹⁴)₂, —NO₂, ═O, and —CN; and
C₃₋₁₀ carbocycle and 3- to 10-membered heterocycle, each of which are optionally substituted with halogen, —OR¹⁴, —N(R¹⁴)₂, —C(O)R¹⁴, —C(O)N(R¹⁴)₂, —NO₂, ═O, —CN, C₁₋₆ alkyl, and C₁₋₆ haloalkyl.

8. The compound or salt of claim 1, wherein one of R¹ and R² is selected from hydrogen, methyl, ethyl, and propyl, and the other is selected from methyl, ethyl, propyl,

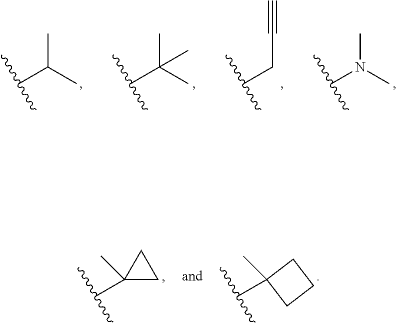

9. The compound or salt of claim 1, wherein R¹ and R² come together to form a 3- to 10-membered heterocycle optionally substituted with one or more substituents independently selected from halogen, —OR¹⁵, —N(R¹⁵)₂, —NO₂, ═O, —CN, C₁₋₆ alkyl, and C₁₋₆ haloalkyl.

10. The compound or salt of claim 1, wherein 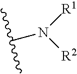
is selected from

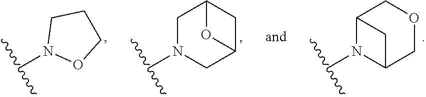 [SMILES: *N1CCCO1 ; *N1CC2CC(C1)O2 ; *N1C2COCC1C2]

11. The compound or salt of claim 1, wherein L⁴ is absent and each of L¹, L², and L³ are independently selected from (a) and (b):
(a) —O—, —N(R¹²)—, —S—, —N(R¹²)C(O)—, and —N(R¹²)C(O)O—; and
(b) C₁₋₆ alkylene optionally substituted with one or more substituents independently selected from halogen, —OR¹³, —N(R¹³)₂, and —CN.

12. The compound or salt of claim 1, wherein L is selected from

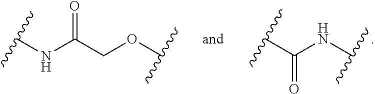 [SMILES: *NC(=O)CO* ; *NC(*)=O]

13. The compound or salt of claim 1, wherein R³ is hydrogen.

14. The compound or salt of claim 1, wherein R⁴ is independently selected from halogen, —OR¹¹, —N(R¹¹)₂, —NO₂, —CN, C₁₋₆ alkyl, and C₁₋₆ haloalkyl.

15. A compound represented by the structure of Formula (III): 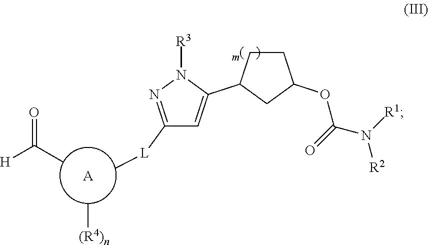
or a pharmaceutically acceptable salt thereof wherein:
Ring A is selected from C₃₋₁₂ carbocycle and 3- to 12-membered heterocycle;
each R⁴ is independently selected at each occurrence from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₂₋₆ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₃₋₁₀ carbocycle and 3- to 10-membered heterocycle, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN;
L is represented by -L¹-L²-L³-L⁴-, wherein L¹, L², L³, and L⁴ are each independently selected from (a) and (b):
(a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
(b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³ ═O, ═S, —CN, C₁₋₆ alkyl, and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN;
wherein L², L³, and L⁴ are each optionally absent;
wherein no more than two of L¹, L², L³, and L⁴ are selected from (a) and the two selected are not adjacent;
R¹ and R² are each independently selected from (i), (ii), and (iii):
(i) hydrogen, —OR¹⁴, —N(R¹⁴)₂, —C(O)R¹⁴, —C(O)N(R¹⁴)₂, —C(O)OR¹⁴, —OC(O)R¹⁴, —N(R¹⁴)C(O)OR¹⁴, —N(R¹⁴)C(O)R¹⁴, —S(O)R¹⁴, —S(O)₂R¹⁴, —N(R¹⁴)S(O)₂R¹⁴, —S(O)₂N(R¹⁴)₂, and —CN;
(ii) C₁₋₆ alkyl, C₂₋₆ alkenyl, and C₂₋₆ alkynyl, each of which is optionally substituted with one or more substituents independently selected from:
halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
C₃₋₁₀ carbocycle and 3- to 10-membered heterocycle, each of which is optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; and
(iii) C₃₋₁₀ carbocycle and 3- to 10-membered heterocycle, each of which is optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), —CN; and C₁₋₆ alkyl, C₂₋₆ alkenyl, and C₂₋₆ alkynyl, the C₁₋₆ alkyl, C₂₋₆ alkenyl, and C₂₋₆ alkynyl are each optionally substituted with one or more substituents independently selected from halogen, —OR¹⁴, —SR¹⁴, —N(R¹⁴)₂, —C(O)R¹⁴, —C(O)N(R¹⁴)₂, —N(R¹⁴)C(O)R¹⁴, —C(O)OR¹⁴, —OC(O)R¹⁴, —N(R¹⁴)C(O)OR¹⁴, —OC(O)N(R¹⁴)₂, —N(R¹⁴)C(O)N(R¹⁴)₂, —S(O)R¹⁴, —S(O)₂R¹⁴, —N(R¹⁴)S(O)₂R¹⁴, —S(O)₂N(R¹⁴)₂, —NO₂, ═O, ═S, ═N(R¹⁴), and —CN; or
R¹ and R² may come together to form a 3- to 10-membered heterocycle optionally substituted with one or more substituents independently selected from:
halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN; and
C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from halogen, —OR¹⁵, —SR¹⁵, —N(R¹⁵)₂, —C(O)R¹⁵, —C(O)N(R¹⁵)₂, —N(R¹⁵)C(O)R¹⁵, —C(O)OR¹⁵, —OC(O)R¹⁵, —N(R¹⁵)C(O)OR¹⁵, —OC(O)N(R¹⁵)₂, —N(R¹⁵)C(O)N(R¹⁵)₂, —S(O)R¹⁵, —S(O)₂R¹⁵, —N(R¹⁵)S(O)₂R¹⁵, —S(O)₂N(R¹⁵)₂, —NO₂, ═O, ═S, ═N(R¹⁵), and —CN;
R³ is selected from hydrogen and C₁₋₆ alkyl optionally substituted with one or more substituents independently selected from halogen, —OR¹⁶, —SR¹⁶, —N(R¹⁶)₂, —C(O)R¹⁶, —C(O)N(R¹⁶)₂, —C(O)OR¹⁶, —NO₂, and —CN;
R¹¹, R¹², R¹³, R¹⁴, R, and R¹⁶ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle, and 3- to 10-membered heterocycle, wherein each C₃₋₁₀ carbocycle and 3- to 10-membered heterocycle are optionally substituted with one or more substituents independently selected from halogen, —OH, —O—C₁₋₆ alkyl, —O—C₁₋₆ haloalkyl, —NH₂, —NO₂, ═O, and —CN; and
C₃₋₆ carbocycle and 3- to 6-membered heterocycle optionally substituted with one or more substituents independently selected from halogen, —OH, —O—C₁₋₆ alkyl, —O—C₁₋₆ haloalkyl, —NH₂, —NO₂, ═O, and —CN;
n is selected from 0, 1, 2, 3, and 4; and
m is selected from 0 and 1.

16. The compound or salt of claim 15, wherein 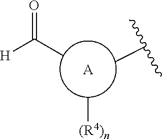
is selected from

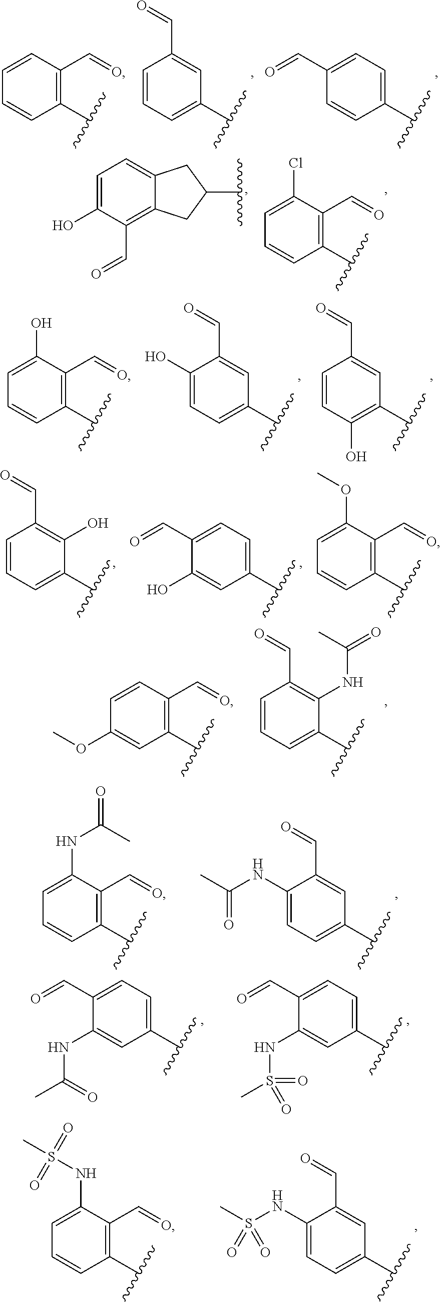

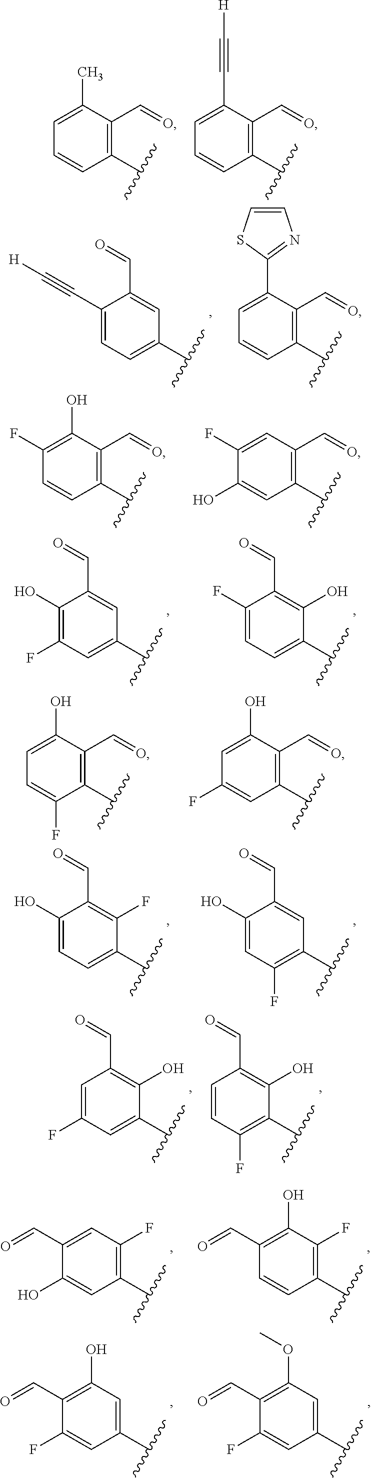

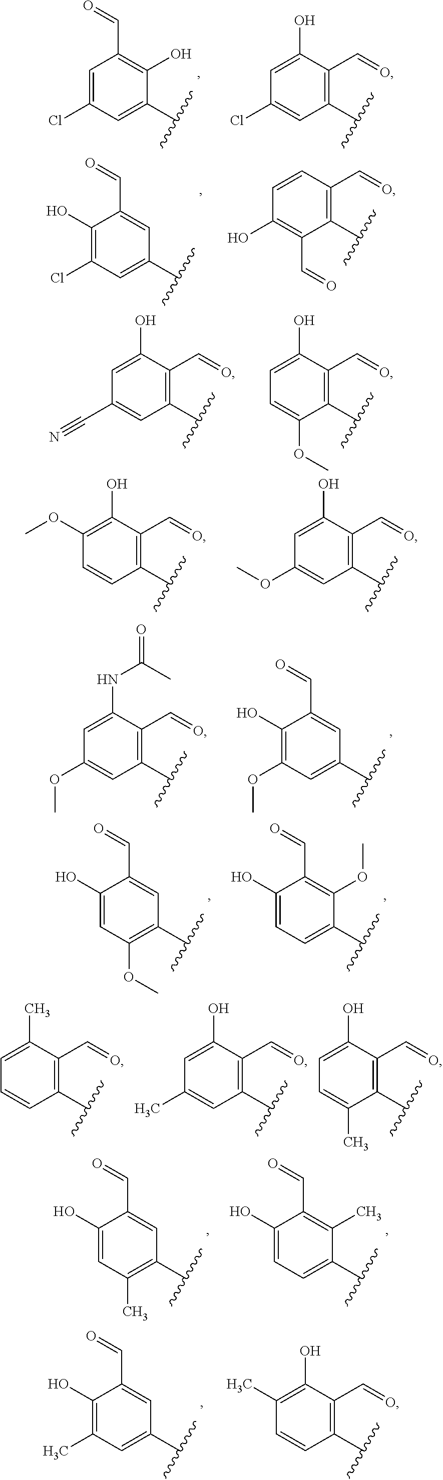

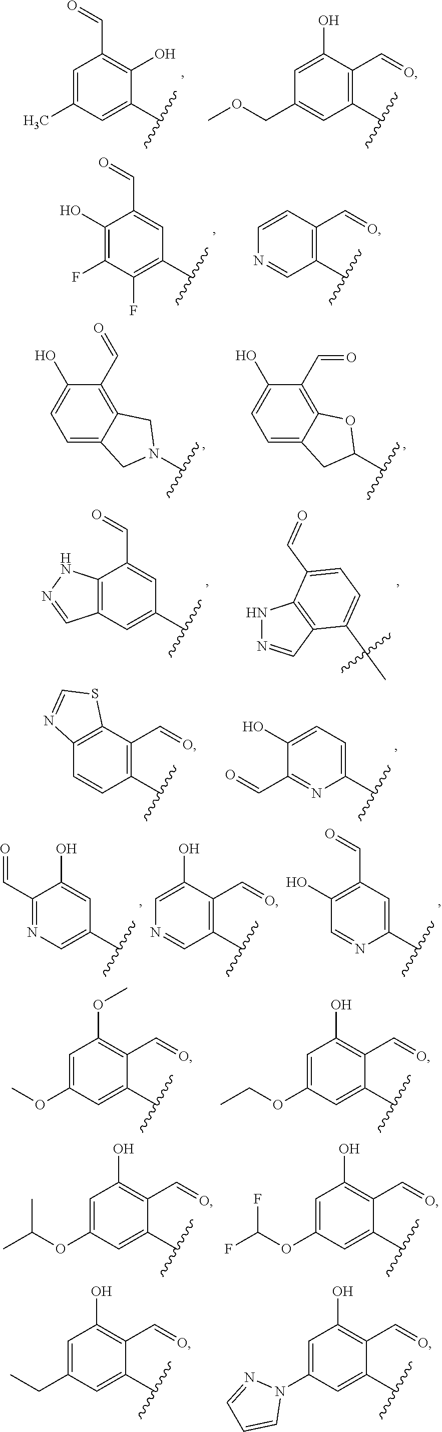

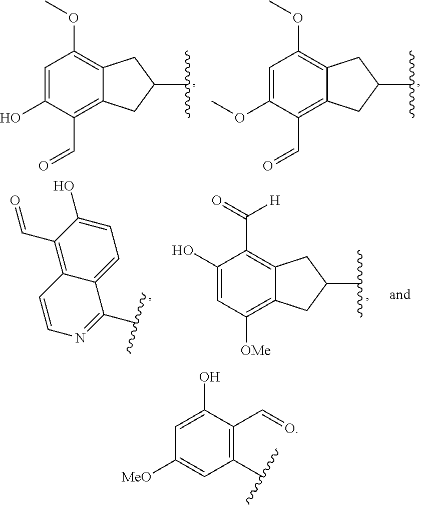

17. The compound or salt of claim 15, wherein one of R¹ and R² is selected from hydrogen, C₁₋₆ alkyl, and C₁₋₆ haloalkyl, and the other is selected from:
—OR¹⁴, —N(R¹⁴)₂, —C(O)R¹⁴, —C(O)N(R¹⁴)₂, —CN, C₁₋₆ alkyl, C₂₋₆ alkenyl, and C₂₋₆ alkynyl, wherein the C₁₋₆ alkyl, C₂₋₆ alkenyl, and C₂₋₆ alkynyl are each optionally substituted with one or more substituents independently selected from halogen, —OR¹⁴, —N(R¹⁴)₂, —C(O)R¹⁴, —C(O)N(R¹⁴)₂, —NO₂, ═O, and —CN; and
C₃₋₁₀ carbocycle and 3- to 10-membered heterocycle, each of which are optionally substituted with halogen, —OR¹⁴, —N(R¹⁴)₂, —C(O)R¹⁴, —C(O)N(R¹⁴)₂, —NO₂, ═O, —CN, C₁₋₆ alkyl, and C₁₋₆ haloalkyl.

18. The compound or salt of claim 15, wherein one of R¹ and R² is selected from hydrogen and the other of R¹ or R² is selected from —OH, —OMe, —NMe₂,

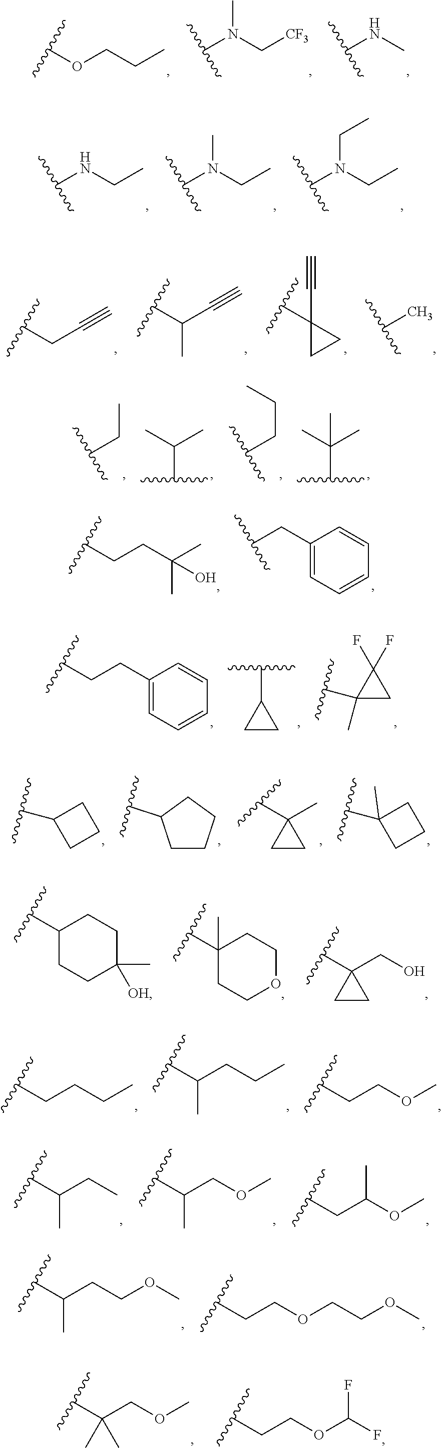

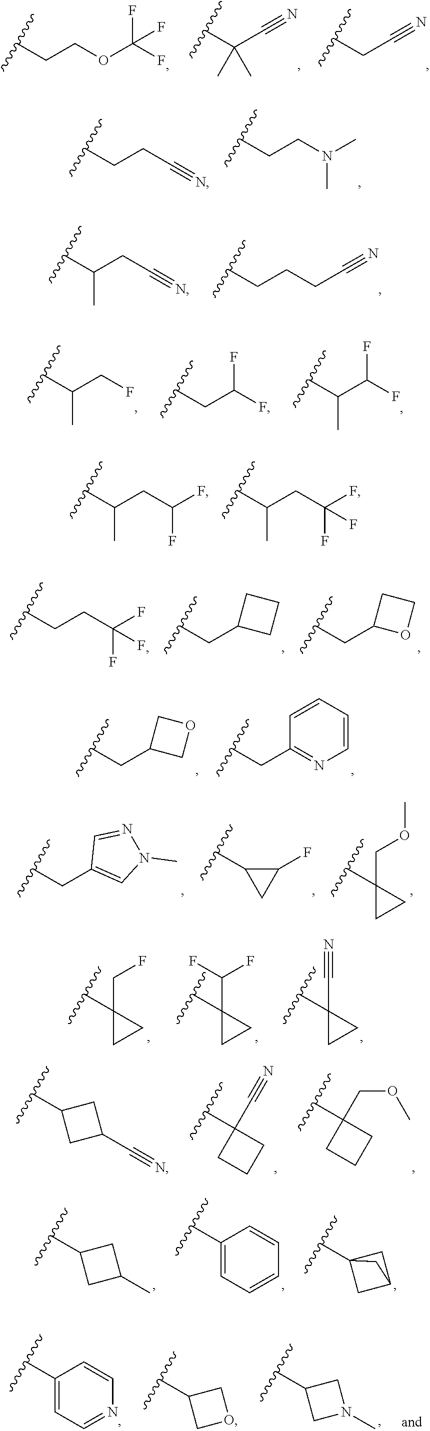

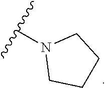 [SMILES: *N1CCCC1]

19. The compound or salt of claim 15, wherein 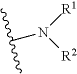
is selected from

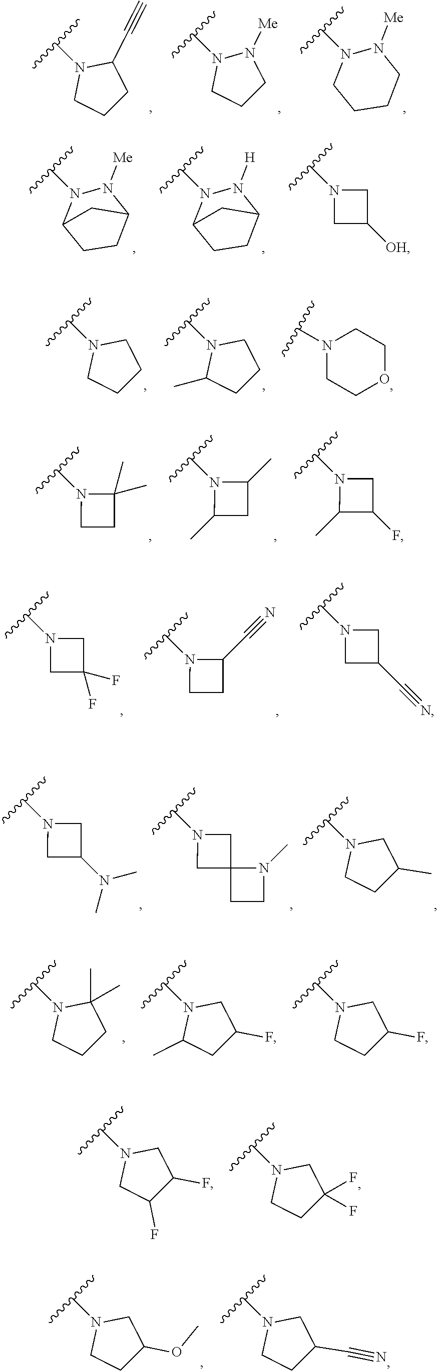

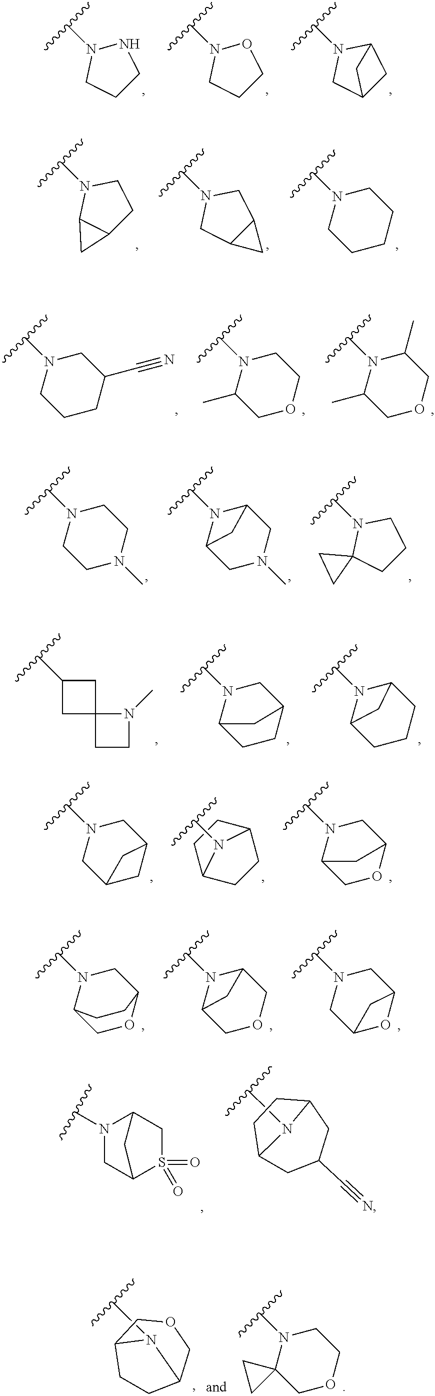

20. The compound or salt of claim 15, wherein L⁴ is absent and each of L¹, L², and L³ are independently selected from (a) and (b):
(a) —O—, —N(R¹²)—, —S—, —S(O)—, —S(O)₂—, —S(O)(NR¹²)—, —N(R¹²)C(O)—, —N(R¹²)C(O)O—, —N(R¹²)C(O)C(O)N(R¹²)—, —N(R¹²)S(O)₂—, —N(R¹²)S(O)₂N(R¹²)—, —S(O)(NR¹²)N(R¹²)—, —N(R¹²)N(R¹²)—, —(R¹²)NC(O)N(R¹²)—, and —(R¹²)NC(O)N(R¹²)N(R¹²)—; and
(b) C₁₋₆ alkylene, C₂₋₆ alkenylene, C₂₋₆ alkynylene, C₃₋₁₀ carbocyclene, and 3- to 10-membered heterocyclene, any one of which is optionally substituted with one or more substituents independently selected from halogen, —OR¹³, —SR¹³, ═O, ═S, —CN, C₁₋₆ alkyl, and C₃₋₆ carbocycle, wherein the C₁₋₆ alkyl and C₃₋₆ carbocycle are each optionally substituted with one or more substituents independently selected from halogen, —OR¹³, —SR¹³, ═O, ═S, and —CN.

21. The compound or salt of claim 15, wherein L is selected from

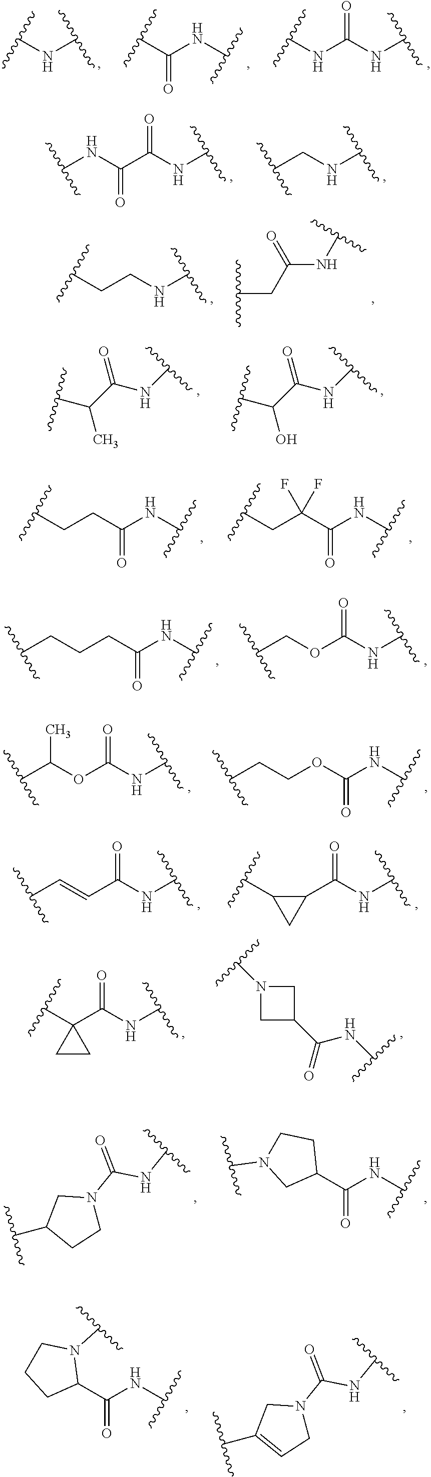

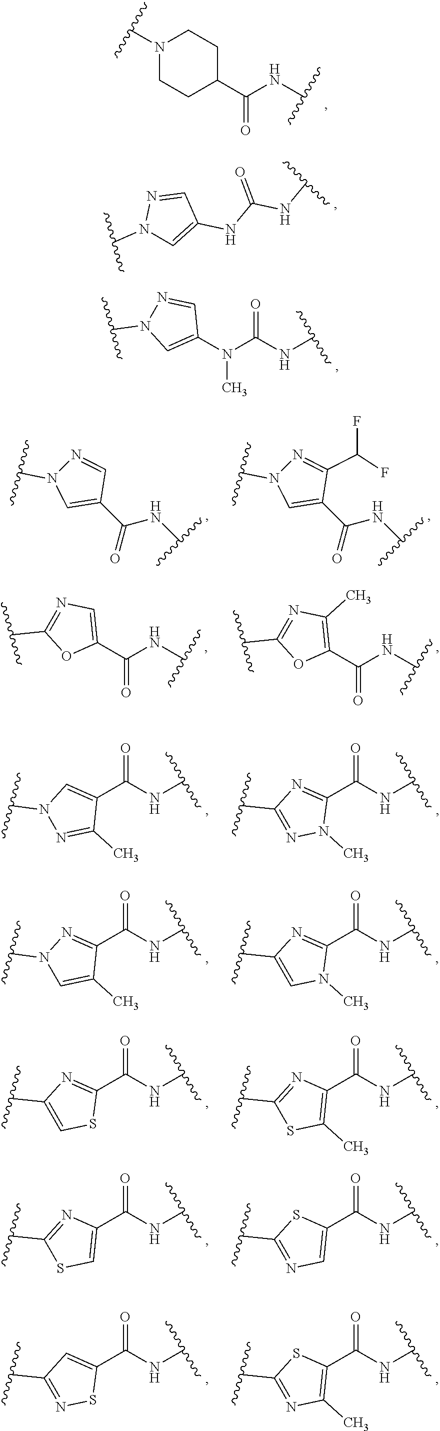

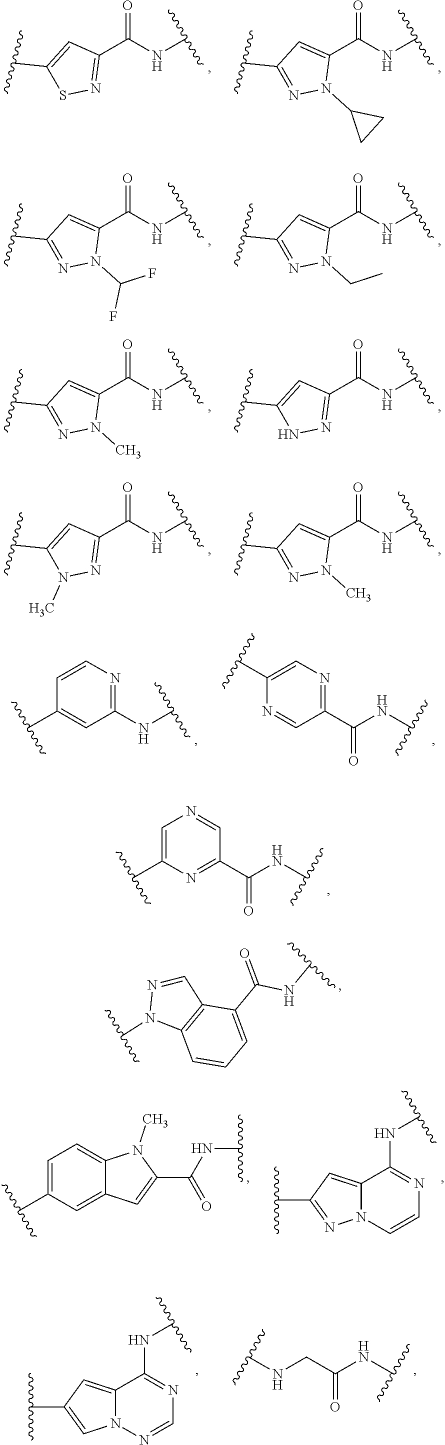

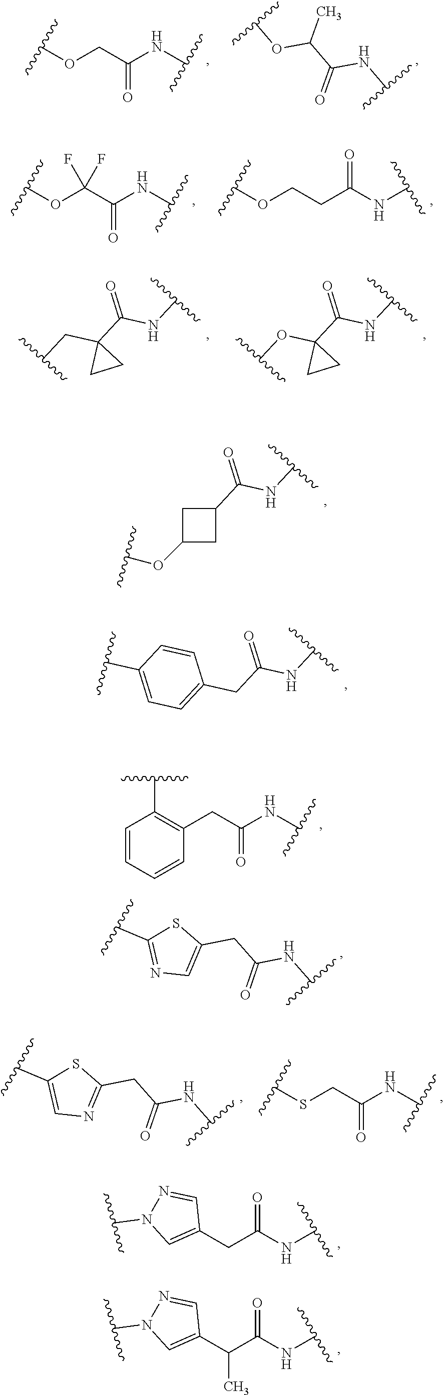

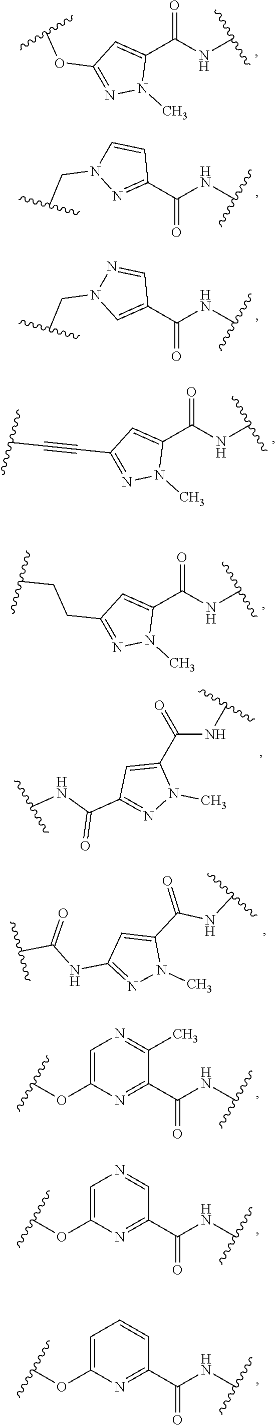

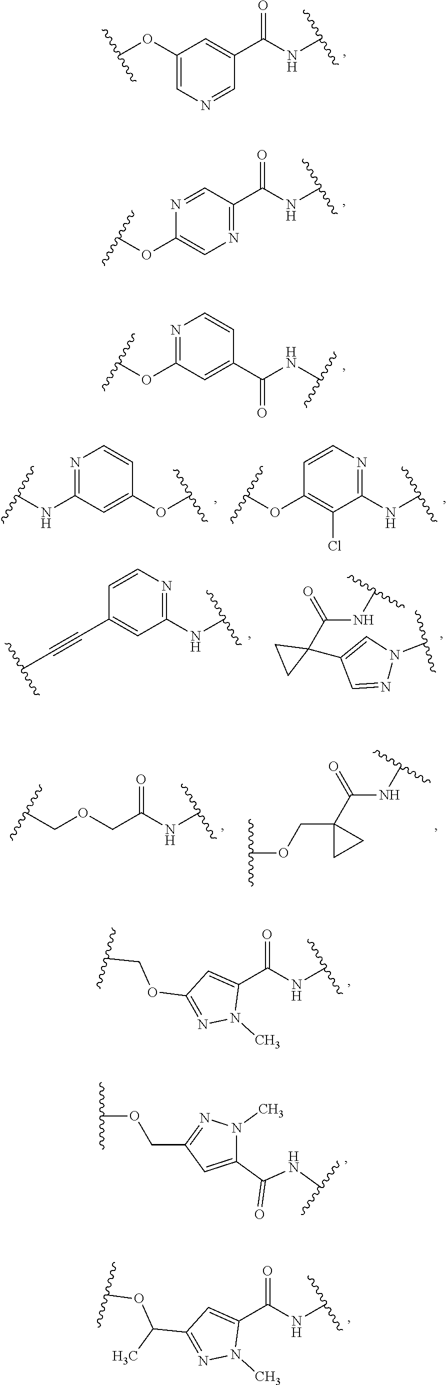

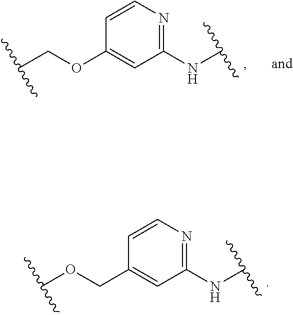 [SMILES: *COc1ccnc(N*)c1 ; *Nc1cc(CO*)ccn1]

22. The compound or salt of claim 15, wherein R³ is selected from hydrogen, C₁₋₆ alkyl, and C₁₋₆ haloalkyl.

23. The compound or salt of claim 15, wherein R⁴ is selected from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN; and
C₁₋₆ alkyl, C₂₋₆ alkenyl, and C₂₋₆ alkynyl, each of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —N(R¹¹)C(O)OR¹¹, —OC(O)N(R¹¹)₂, —N(R¹¹)C(O)N(R¹¹)₂, —S(O)R¹¹, —S(O)₂R¹¹, —N(R¹¹)S(O)₂R¹¹, —S(O)₂N(R¹¹)₂, —NO₂, ═O, ═S, ═N(R¹¹), and —CN.

24. The compound or salt of claim 1, wherein the compound of Formula (II) is selected from a compound of Table 1A, or a pharmaceutically acceptable salt of any one thereof.

25. The compound or salt of claim 15, wherein the compound of Formula (III) is selected from a compound of Table 1, or a pharmaceutically acceptable salt of any one thereof.

26. A pharmaceutical composition comprising a compound or salt of claim 1, and a pharmaceutically acceptable excipient.

27. A method of treating a disease or disorder mediated by CDK2 comprising, administering to a subject in need thereof the compound or salt of claim 1, or a pharmaceutical composition comprising a compound or salt of claim 1 and a pharmaceutically acceptable excipient.

28. The method of claim 27, wherein the disease or disorder is cancer.

29. The method of claim 28, wherein the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, and thyroid cancer.

30. The method of claim 29, wherein the administering is selective for CDK2 over a CDK selected from CDK1, CDK4, and CDK6.
